SEQUENCE LISTING (1 sequence)

SEQ ID NO: 1 - protein, 470 aa, synthetic construct
  REGION 1..470: note = hPDL1(18-239)-TVMV-mIgG1(221-447)-C225S
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSPGGGGGRETVRFQGGTGDAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2025/0163104 A1
(43) Pub. Date: May 22, 2025

(54) MACROCYCLIC IMMUNOMODULATORS

(71) Applicant: Bristol-Myers Squibb Company, Princeton, NJ (US)

(72) Inventors: Jennifer X. QIAO, Princeton, NJ (US); Michael A. POSS, Princeton, NJ (US); Martin Patrick ALLEN, Flemington, NJ (US); Claudio MAPELLI, Linden, NJ (US); Claude A. QUESNELLE, Skillman, NJ (US); David R. TORTOLANI, Princeton, NJ (US); Tammy C. WANG, Lawrenceville, NJ (US); Tao WANG, Glastonbury, CT (US); Yong ZHANG, Princeton, NJ (US); Yunhui ZHANG, Princeton, NJ (US); Zhongxing ZHANG, Madison, CT (US)

(21) Appl. No.: 18/715,382

(22) PCT Filed: Dec. 2, 2022
(86) PCT No.: PCT/US2022/080790

§ 371 Date: May 31, 2024

Related U.S. Application Data
(60) Provisional application No. 63/285,826, filed on Dec. 3, 2021.

(51) Int. Cl. C07K 7/56; A61K 38/12

(52) U.S. Cl. CPC C07K 7/56; A61K 38/12

(57) ABSTRACT

In accordance with the present disclosure, macrocyclic compounds have been discovered that bind to PD-I and are capable of inhibiting the interaction of PD-I with PD-LI. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims the priority benefit of U.S. Provisional Application No. 63/285,826, filed Dec. 3, 2021, which is incorporated herein by reference in its entirety.

### REFERENCE TO SEQUENCE LISTING SUBMITTED ELECTRONICALLY

The content of the electronically submitted sequence listing (Name: “3338_221PC01_Seglisting_ST26.xml”; Size: 2,577 bytes; and Date of Creation: Dec. 1, 2022) is herein incorporated by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med, 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med, 192:1027-1034 (2000); Latchman et al., Nat. Immunol., 2:261-268 (2001); Carter et al., Eur. J Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med, 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med, 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad Sci. USA, 99:12293-12297 (2002); Brown et al., J. Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26:Epub (2008)). Chronic antigen stimulation, such as that which occurs during tumor disease or chronic infections, results in T cells that express elevated levels of PD-I and are dysfunctional with respect to activity towards the chronic antigen (reviewed in Kim et al., Curr. Opin. Imm. (2010)). This is termed “T cell exhaustion”. B cells also display PD-I/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection,” J. Exp. Med., 205(3):543-555 (2008); Finnefrock, A. C. et al., “PD-1 blockade in rhesus macaques: impact on chronic infection and prophylactic vaccination,” J. Immunol., 182(2):980-987 (2009); Song, M.-Y. et al., “Enhancement of vaccine-induced primary and memory CD8+t-cell responses by soluble PD-1,” J. Immunother., 34(3):297-306 (2011)).

The PD-1 pathway is a key inhibitory molecule in T cell exhaustion that arises from chronic antigen stimulation during chronic infections and tumor disease.

Accordingly, agents that block the interaction of PD-1 with PD-L1 are desired.

### SUMMARY

The present disclosure provides macrocyclic compounds which inhibit the PD-1 protein/protein interaction, and are thus useful for the amelioration of various diseases, including cancer and infectious diseases.

In a first aspect the present disclosure provides a compound of Formula (I):

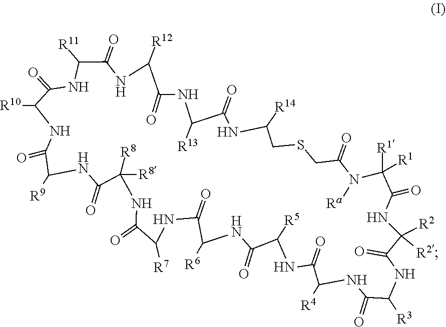

or a pharmaceutically acceptable salt thereof, wherein:
R¹ is selected from C₁-C₆alkoxyC₁-C₆alkyl; C₁-C₆alkyl; C₁-C₆alkylaminoC₁-C₆alkyl; C₁-C₆alkylcarbonylaminoC₁-C₆alkyl; aminoC₁-C₆alkyl; aminocarbonylC₁-C₆alkyl; arylC₁-C₆alkyl; arylcarbonylaminoC₁-C₆alkyl; carboxyC₁-C₆alkyl; cyanoC₁-C₆alkyl; heteroarylC₁-C₆alkyl; heterocyclylC₁-C₆alkyl; hydroxyC₁-C₆alkyl; NH₂C(X)NHC₁-C₆alkyl, wherein X is O or NH; and

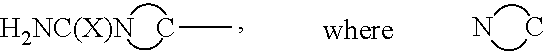

represents an azetidine, piperidine, or pyrrolidine ring; wherein the aryl part of the arylC₁-C₆alkyl and the arylcarbonylaminoC₁-C₆alkyl and the heteroaryl part of the heteroarylC₁-C₆alkyl are optionally substituted with one, two, three, four, or five groups independently selected from C₁-C₆alkoxy, C₁-C₆alkylcarbonylamino, C₂-C₆alkynyloxy, aminocarbonyl, aryl optionally substituted with one or two carboxy groups, arylC₁-C₆alkoxy, carboxy, carboxyC₁-C₆alkoxy, halo, and trifluoromethyl;
R^{1′} is hydrogen or C₁-C₆alkyl;
R² is selected from C₁-C₆alkoxyC₁-C₆alkyl; arylC₁-C₆alkyl; azidoC₁-C₆alkyl; biscarboxyCHC₁-C₆alkyl; carboxyC₁-C₆alkyl; and heteroarylC₁-C₆alkyl; wherein

the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, C₁-C₆alkylcarbonylamino, C₂-C₆alkynyloxy, amino, aminoC₁-C₆alkyl, aminocarbonyl, aryl optionally substituted with one or two carboxy groups, arylC₁-C₆alkoxy, arylcarbonyl, azido, carboxy, carboxyC₁-C₆alkoxy, carboxyC₁-C₆alkyl, cyano, halo, haloC₁-C₆alkoxy, hydroxy, nitro, and trifluoromethyl;
R^{2′} is hydrogen or C₁-C₆alkyl;
R³ is selected from C₁-C₆alkoxyC₁-C₆alkyl; aminocarbonylC₁-C₆alkyl, arylC₁-C₆alkoxyC₁-C₆alkyl, arylC₁-C₃alkyl, carboxyC₁-C₆alkyl, furylC₁-C₃alkyl, hydroxyC₁-C₆alkyl, HOS(O)₂C₁-C₃alkyl, CH₃S(O)₂NHC(O)(C₁-C₃alkyl), and tetrazolylC₁-C₃alkyl; wherein the aryl part of the arylC₁-C₃alkyl is optionally substituted with one, two, or three, aminoC₁-C₃alkyl groups;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one or more groups independently selected from C₁-C₆alkoxy, C₁-C₆alkyl, amino, cyano, C₁-C₆fluoroalkyl, halo, and hydroxy;
R⁵ is selected from C₁-C₆alkoxyC₁-C₆alkyl; C₁-C₆alkyl; aryl; arylC₁-C₆alkyl; cyanoC₁-C₆alkyl; C₃-C₈cycloalkyl; (C₃-C₈cycloalkyl)C₁-C₆alkyl; fluoroC₁-C₆alkyl; heteroarylC₁-C₆alkyl; and hydroxyC₁-C₆alkyl; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, amino, aminoC₁-C₆alkyl, aminocarbonyl, aryl, arylC₁-C₆alkoxy, aryloxy, carboxyC₁-C₆alkoxy, cyano, (C₃-C₆cycloalkyl)oxy, carboxy, halo, heteroaryl, and hydroxy, wherein the aryl is further optionally substituted with one, two, or three groups independently selected from C₁-C₃alkyl, C₁-C₃alkylcarbonylamino, carboxy, and hydroxy;
R⁶ is selected from aryl-arylC₁-C₃alkyl, aryl-heteroarylC₁-C₃alkyl, heteroaryl-arylC₁-C₃alkyl, and heteroaryl-heteroarylC₁-C₃alkyl; wherein each aryl and each heteroaryl are optionally substituted with one or more groups independently selected from C₁-C₆alkoxy, C₁-C₆alkyl, amino, cyano, C₁-C₆fluoroalkyl, halo, and hydroxyl.

R⁷ is selected from hydrogen; C₁-C₆alkyl; C₁-C₆alkylcarbonylaminoC₁-C₆alkyl; C₂-C₆alkynyl; aminoC₁-C₆alkyl; aminocarbonylC₁-C₆alkyl; aryl; arylC₁-C₆alkyl; carboxyC₁-C₆alkyl; C₃-C₈cycloalkyl; (C₃-C₈cycloalkyl)C₁-C₆alkyl; haloarylcarbonylaminoC₁-C₆alkyl; heteroarylC₁-C₆alkyl; hydroxyC₁-C₆alkyl; and NH₂C(X)NHC₁-C₆alkyl, where X is O or NH; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, arylC₁-C₆alkoxy, carboxy, carboxyC₁-C₆alkoxy, haloC₁-C₆alkoxy, and hydroxy;
R⁸ is selected from C₁-C₆alkyl; C₁-C₆alkylcarbonylaminoC₁-C₆alkyl; aminoC₁-C₆alkyl; (C₇H₁₅O₆)aminoC₁-C₆alkyl; aminocarbonylC₁-C₆alkyl; arylC₁-C₆alkyl; carboxyC₁-C₆alkyl; heterocyclyl; heteroarylC₁-C₆alkyl; and hydroxyC₁-C₆alkyl; wherein the aryl part of the arylC₁-C₆alkyl and the arylcarbonylaminoC₁-C₆alkyl are optionally substituted with one, two, three, four, or five groups independently selected from aminoC₁-C₆alkyl, halo, and hydroxy;
R^{8′} is hydrogen or R⁸ and R^{8′}, together with the atoms to which they are attached, form a C₃-C₈cycloalkyl ring;
R⁹ is selected from C₁-C₆alkyl; arylC₁-C₆alkyl; and C₃-C₈cycloalkylC₁-C₆alkyl; wherein the aryl part of the arylC₁-C₆alkyl is optionally substituted with one, two, three, four, or five groups independently selected from halo and hydroxy;
R¹⁰ is selected from C₁-C₆alkyl; C₂-C₆alkynyl; aminoC₁-C₆alkyl; aminocarbonylC₁-C₆alkyl; arylC₁-C₆alkyl; carboxyC₁-C₆alkyl; hydroxyC₁-C₆alkyl; (C₇H₁₅O₆)aminoC₁-C₆alkyl; C₁-C₆alkylcarbonylaminoC₁-C₆alkyl; heteroarylC₁-C₆alkyl; and NH₂C(X)NHC₁-C₆alkyl, wherein X is O or NH; wherein the aryl part of the arylC₁-C₆alkyl is optionally substituted with one, two, three, four, or five groups independently selected from C₂-C₆alkynyloxy, and arylC₁-C₆alkoxy;
R¹¹ is selected from C₁-C₈alkyl; arylC₁-C₆alkyl; C₃-C₈cycloalkylC₁-C₆alkyl; and heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₁-C₆alkoxy, aminoC₁-C₆alkyl cyano, halo, hydroxy, and trifluoromethyl;
R¹² is selected from C₁-C₆alkyl, C₂-C₆alkynyl, arylC₁-C₆alkyl, carboxyC₁-C₆alkyl, and hydroxyC₁-C₆alkyl; wherein the aryl part of the arylC₁-C₆alkyl is optionally substituted with one, two, three, four, or five groups independently selected from C₂-C₆alkynyloxy and arylC₁-C₆alkoxy;
R¹³ is selected from C₁-C₆alkyl, C₁-C₆alkylcarbonylaminoC₁-C₆alkyl, aminoC₁-C₆alkyl, aminocarbonylC₁-C₆alkyl, arylC₁-C₆alkyl, carboxyC₁-C₆alkyl, haloarylcarbonylaminoC₁-C₆alkyl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl is optionally substituted with one, two, three, four, or five groups independently selected from C₂-C₆alkynyloxy and arylC₁-C₆alkoxy;
R¹⁴ is aminocarbonyl; carboxy; or —C(O)NR^{14′}CR¹⁵R^{15′}R^{15″}, wherein
R^{14′} is hydrogen or C₁-C₆alkyl, or R¹⁵ and R^{14′}, together with the atoms to which they are attached, form an azetidine, morpholine, piperazine, piperidine, or pyrrolidine ring, wherein each ring is optionally substituted with an amino or a hydroxy group;
R¹⁵ is selected from hydrogen, C₁-C₆alkyl, C₁-C₆alkylcarbonylaminoC₁-C₆alkyl, C₂-C₆alkynyl, aminoC₁-C₆alkyl, aminocarbonylC₁-C₆alkyl, arylC₁-C₆alkyl, azidoC₁-C₆alkyl, carboxy, carboxyC₁-C₆alkyl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl is optionally substituted with one, two, three, four, or five groups independently selected from arylC₁-C₆alkoxy and hydroxy;
R^{15′} is hydrogen or C₁-C₆alkyl; or R¹⁵ and R^{15′}, together with the atoms to which they are attached, form a C₃-C₈cycloalkyl ring; and
R^{15″} is hydrogen; amincarbonyl, carboxy, or —(CH₂)ₙC(O)NHCHR¹⁶R^{16′}; wherein

n is 0, 1, or 2;
R¹⁶ is selected from hydrogen, C₁-C₆alkoxyC₁-C₆alkyl, C₁-C₆alkylC₂-C₆alkynyl, C₂-C₆alkynyl, aminoC₁-C₆alkyl, arylC₁-C₆alkyl, carboxy, carboxyC₁-C₆alkyl, heteroaryl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl and the heteroaryl are optionally substituted with one, two, three, four, or five groups independently selected from C₁-C₆alkyl, arylC₁-C₆alkoxy, and hydroxy; and
R^{16′} is hydrogen, C₁-C₆alkyl, aminocarbonyl, carboxy, or —(CH₂)ₘC(O)NHCHR¹⁷R^{17′}; wherein
m is 0, 1, or 2;
R¹⁷ is C₂-C₆alkynyl; and
R^{17′} is aminocarbonyl or carboxy; and
R^{a} is hydrogen or C₁-C₆alkyl; or R¹ and R^{a}, together with the atoms to which they are attached, form an azetidine, morpholine, piperidine, piperazine, or pyrrolidine ring, wherein each ring is optionally substituted with an amino or a hydroxy group.

In some aspects, R¹ is selected from C₁-C₆alkyl, aminoC₁-C₆alkyl, aminocarbonylC₁-C₆alkyl, arylC₁-C₆alkyl, heteroarylC₁-C₆alkyl, heterocyclylC₁-C₆alkyl, and hydroxyC₁-C₆alkyl, wherein the aryl part of the arylC₁-C₆alkyl is optionally substituted with one, two, or three groups independently selected from halo and carboxyC₁-C₆alkoxy; and
R^{1′} is hydrogen.

In some aspects, R² is selected from arylC₁-C₆alkyl and heteroarylC₁-C₆alkyl, wherein the aryl part of the arylC₁-C₆alkyl is optionally substituted with one, two, or three groups independently selected from carboxy, carboxyC₁-C₆alkoxy, cyano, halo, hydroxy, and nitro; and R^{2′} is hydrogen.

In some aspects, R³ is aminocarbonylC₁-C₆alkyl or carboxyC₁-C₆alkyl.

In some aspects, R⁴ is arylC₁-C₆alkyl; wherein the aryl part of the arylC₁-C₆alkyl is optionally substituted with one, two, or three groups independently selected from C₁-C₆alkyl, halo, and trifluoromethyl.

In some aspects, R⁵ is C₁-C₆alkyl or arylC₁-C₆alkyl; wherein the aryl part of the arylC₁-C₆alkyl is optionally substituted with one, two, or three groups independently selected from carboxy, carboxyC₁-C₆alkoxy, and hydroxy.

In some aspects, R⁶ is biphenylC₁-C₆alkyl.

In some aspects, R⁷ is selected from C₁-C₆alkyl, arylC₁-C₆alkyl, carboxyC₁-C₆alkyl, and NH₂C(X)NHC₁-C₆alkyl, wherein X is O or NH; wherein the aryl part of the arylC₁-C₆alkyl is optionally substituted with one, two, or three groups independently selected from carboxy, carboxyC₁-C₆alkoxy and hydroxy.

In some aspects, R⁸ is C₁-C₆alkyl or aminoC₁-C₆alkyl; and R^{8′} is hydrogen.

In some aspects, R⁹ is C₁-C₆alkyl.

In some aspects, R¹⁰ is aminoC₁-C₆alkyl or aminocarbonylC₁-C₆alkyl.

In some aspects, R¹¹ is C₁-C₆alkyl or C₃-C₆cycloalkylC₁-C₃alkyl.

In some aspects, R¹² is C₁-C₄alkyl or hydroxyC₁-C₄alkyl.

In some aspects, R¹³ is aminoC₁-C₆alkyl, aminocarbonylC₁-C₂alkyl, carboxyC₁-C₆alkyl, or hydroxyC₁-C₄alkyl.

In some aspects, R¹⁴ is aminocarbonyl or —C(O)NHCHR¹⁵C(O)NH₂, and wherein R¹⁵ is hydrogen, C₁-C₆alkyl, aminoC₁-C₆alkyl.

In some aspects, R¹⁵ is hydrogen or C₁-C₆alkyl.

In some aspects, R¹⁶ is hydrogen or C₂-C₄alkynyl.

In some aspects, R^{a} is methyl.

In some aspects, one, two, or all of R^{1′}, R^{2′}, and R^{8′} are methyl.

In some aspects, the present disclosure provides compounds of Formula (I), or a pharmaceutically acceptable salt thereof, wherein:
R¹ is selected from C₁-C₄alkyl, C₁-C₄alkylcarbonylaminoC₂-C₄alkyl, aminoC₁-C₃alkyl, aminocarbonylC₁-C₂alkyl, arylC₁-C₂alkyl, arylcarbonylaminoC₁-C₂alkyl, carboxypropyl, cyanomethyl; heteroarylmethyl, heterocyclylmethyl, hydroxyC₂-C₃alkyl, methoxyC₁-C₂alkyl, methylaminoC₁-C₂alkyl, NH₂C(X)NHpropyl, wherein X is O or NH, and H₂NC(X)piperidinyl, wherein the aryl part of the arylC₁-C₂alkyl and the arylcarbonylaminoC₁-C₆alkyl is optionally substituted with one, two, or three groups independently selected from aminocarbonyl, arylmethoxy, carboxy, carboxymethoxy, carboxyphenyl, halo, methoxy, methylcarbonylamino, propynyloxy, and trifluoromethyl;
R^{1′} is hydrogen or methyl;
R² is selected from arylC₁-C₂alkyl, azidoC₁-C₂alkyl, biscarboxyethyl, carboxyC₁-C₃alkyl, methoxyC₁-C₂alkyl, and heteroarylC₁-C₂alkyl; wherein the aryl part of the arylC₁-C₂alkyl and the heteroaryl part of the heteroarylC₁-C₂alkyl are optionally substituted with one, two, three, four, or five groups independently selected from C₁-C₄alkyl, amino, aminocarbonyl, aminomethyl, arylcarbonyl, arylmethoxy, azido, carboxy, carboxymethoxy, carboxymethyl, carboxyphenyl, cyano, halo, hydroxy, methoxy, methylcarbonylamino, nitro, propynyloxy, trifluoromethoxy, and trifluoromethyl;
R^{2′} is hydrogen or methyl;
R³ is selected from aminocarbonylmethyl, arylC₁-C₃alkyl, arylmethoxymethyl, carboxyC₁-C₂alkyl, furylC₁-C₃alkyl, hydroxyC₁-C₂alkyl, methoxymethyl, and tetrazolylmethyl, HOS(O)₂C₁-C₃alkyl, and CH₃S(O)₂NHC(O)(C₁-C₃alkyl); wherein the aryl part of the arylC₁-C₃alkyl is optionally substituted with one, two, or three aminoC₁-C₃alkyl groups;
R⁴ is selected from arylC₁-C₂alkyl and heteroarylmethyl; wherein the aryl part of the arylC₁-C₂alkyl and the heteroaryl part of the heteroarylmethyl are optionally substituted with one, two, three, four, or five groups independently selected from amino, cyano, halo, hydroxy, methoxy, methyl, and trifluoromethyl;
R⁵ is selected from C₁-C₅alkyl, arylmethyl, cyanomethyl, C₃-C₆cycloalkyl, (C₃-C₆cycloalkyl)methyl, heteroarylmethyl, hydroxyC₁-C₂alkyl, methoxymethyl, and phenyl; wherein the aryl part of the arylmethyl is optionally substituted with one, two, three, four, or five groups independently selected from amino, aminomethyl, aminocarbonyl, aryl, arylmethoxy, aryloxy, carboxymethoxy, carboxy, cyano, (C₃-C₆cycloalkyl)oxy, halo, heteroaryl, hydroxy, and propynyloxy; wherein the aryl is further optionally substituted with one, two, or three groups independently selected from C₁-C₃alkyl, C₁-C₃alkylcarbonylamino, carboxy, and hydroxy;
R⁶ is biphenylmethyl;
R⁷ is selected from hydrogen, C₁-C₅alkyl, aminoC₁-C₄alkyl, aminocarbonylethyl, aminocarbonylmethyl, arylmethyl, butynyl, C₃-C₆cycloalkyl, (C₃-C₆cycloalkyl)methyl, carboxyethyl, haloarylcarbonylaminopropyl, heteroarylpropyl, hydroxyC₂alkyl, methylcarbonylaminoC₂-C₄alkyl, phenyl, and NH₂C(X)NHC₂-C₄alkyl, wherein X is O or NH; wherein the aryl part of the arylmethyl is optionally substituted with one, two, or three groups independently selected from arylmethoxy, carboxy, carboxymethoxy, hydroxy, propynyloxy, and trifluoromethoxy;
R⁸ is selected from C₁-C₄alkyl, C₁-C₄alkylcarbonylaminoC₁-C₄alkyl, aminoC₁-C₄alkyl, aminocarbonylC₁-C₂alkyl, (C₇H₁₅O₆)aminomethyl, arylmethyl, carboxyC₁-C₃alkyl, haloarylcarbonylaminopropyl, heterocyclyl, or heteroarylmethyl, and hydroxymethyl; wherein the aryl part of the arylmethyl is optionally substituted with one, two, or three groups independently selected from hydroxy and aminomethyl;
R^{8′} is hydrogen or R⁸ and R^{8′}, together with the atoms to which they are attached, form a cyclopropyl ring;
R⁹ is selected from C₁-C₄alkyl, arylmethyl, and cyclohexylmethyl; wherein the aryl part of the arylmethyl is optionally substituted with one, two, or three hydroxy groups;
R¹⁰ is selected from C₁-C₄alkyl, aminoC₁-C₄alkyl, aminocarbonylC₁-C₂alkyl, (C₇H₁₅O₆)aminomethyl, arylmethyl, butylcarbonylaminoethyl, butynyl, carboxyC₁-C₃alkyl, heteroarylmethyl, hydroxyC₁-C₂alkyl, NH₂C(NH)NHmethyl; wherein the aryl part of the arylmethyl is optionally substituted with one, two, or three groups independently selected from arylmethoxy and propynyloxy;
R¹¹ is selected from C₄-C₈alkyl, arylC₁-C₂alkyl, C₃-C₆cycloalkylC₁-C₂alkyl, and heteroarylmethyl; wherein the aryl part of the arylC₁-C₂alkyl is optionally substituted with one, two, or three groups independently selected from amino, aminoethoxy, aminomethyl, cyano, halo, hydroxy, methoxy, methyl, and trifluoromethyl;
R¹² is selected from C₃-C₄alkyl, arylmethyl, carboxybutyl, hydroxyC₁-C₃alkyl, and propynyl; wherein the aryl part of the arylmethyl is optionally substituted with one, two, or three groups independently selected from arylmethoxy and propynyloxy;
R¹³ is selected from C₃-C₄alkyl, C₁-C₄alkylcarbonylaminoC₂-C₄alkyl, aminoC₁-C₄alkyl, aminocarbonylC₁-C₂alkyl, arylmethyl, carboxyC₁-C₃alkyl, haloarylcarbonylaminopropyl, heteroarylmethyl, hydroxyC₁-C₃alkyl, and NH₂C(X)NHpropyl, wherein X is O or NH; wherein the aryl part of the arylmethyl is optionally substituted with one, two, or three groups independently selected from arylmethoxy and propynyloxy;
R¹⁴ is aminocarbonyl; carboxy; or —C(O)NR^{14′}CR¹⁵R^{15′}R^{15″}; wherein
R^{14′} is hydrogen or methyl; or R¹⁵ and R^{14′}, together with the atoms to which they are attached, form a pyrrolidine ring;
R¹⁵ is selected from hydrogen, C₁-C₂alkyl, C₁-C₄alkylcarbonylaminoC₁-C₃alkyl, aminoC₁-C₄alkyl, aminocarbonylC₁-C₂alkyl, arylmethyl, azidoC₂-C₄alkyl, carboxy, carboxyC₁-C₃alkyl, heteroarylmethyl, hydroxymethyl, NH₂C(NH)NHpropyl, and propynyl; and wherein the aryl part of the arylmethyl is optionally substituted with one, two, or three groups independently selected from arylmethoxy and hydroxy;
R^{15′} is hydrogen or methyl; or R¹⁵ and R^{15′}, together with the atoms to which they are attached, form a cyclopropyl ring; and
R^{15″} is hydrogen, aminocarbonyl, or carboxy; or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxy, carboxyethyl, heteroaryl, heteroarylethyl, hydroxymethyl, methoxymethyl, NH₂C(NH)NHpropyl, and propynyl; wherein the aryl part of the arylC₁-C₆alkyl and the heteroaryl are optionally substituted with one, two, three, four, or five groups independently selected from arylmethoxy, hydroxy, and methyl; and
R^{16′} is hydrogen, aminocarbonyl, carboxy, methyl, or —(CH₂)ₘC(O)NHCHR¹⁷R^{17′}; wherein
m is 0, 1, or 2;
R¹⁷ is propynyl; and
R^{17′} is aminocarbonyl or carboxy; and
R^{a} is hydrogen or methyl; or R¹ and R^{a}, together with the atoms to which they are attached, form a piperazine or pyrrolidine ring, wherein each ring is optionally substituted with an amino or a hydroxy group.

In some aspects, R¹ is selected from C₁-C₆alkyl, aminoC₁-C₃alkyl, aminocarbonylC₁-C₂alkyl, arylC₁-C₂alkyl, heteroarylmethyl, heterocyclulC₁-C₆alkyl, and hydroxyC₂-C₃alkyl, and wherein the aryl part of the arylC₁-C₂alkyl is optionally substituted with one, two, or three groups independently selected from carboxymethoxy and halo; and R^{1′} is hydrogen.

In some aspects, R^{a} is hydrogen.

In some aspects, the present disclosure provides a compound of Formula (I), or a pharmaceutically acceptable salt thereof, wherein:
R¹ is selected from C₁-C₄alkyl, C₁-C₄alkylcarbonylaminoC₂-C₄alkyl, aminoC₁-C₃alkyl, aminocarbonylC₁-C₂alkyl, arylC₁-C₂alkyl, arylcarbonylaminoC₁-C₂alkyl, carboxypropyl, cyanomethyl, heteroarylmethyl, heterocyclmethyl, hydroxyC₂-C₃alkyl, methoxyC₁-C₂alkyl, methylaminoC₁-C₂alkyl, NH₂C(NH)NHpropyl, and H₂NC(NH)piperidinyl; wherein the aryl part of the arylC₁-C₂alkyl and the arylcarbonylaminoC₁-C₂alkyl is optionally substituted with one, two, or three groups independently selected from aminocarbonyl, carboxy, carboxymethoxy, halo, methoxy, methylcarbonylamino, propynyloxy, and trifluoromethyl;
R^{1′} is hydrogen;
R² is selected from arylC₁-C₂alkyl, azidoC₁-C₂alkyl, carboxypropyl, heteroarylC₁-C₂alkyl, and methoxyC₁-C₂alkyl; wherein the aryl part of the arylC₁-C₂alkyl and the heteroaryl part of the heteroarylC₁-C₂alkyl are optionally substituted with one or more groups independently selected from C₁-C₄alkyl, amino, aminocarbonyl, aminomethyl, arylcarbonyl, arylmethoxy, carboxy, carboxymethoxy, carboxymethyl, cyano, halo, hydroxy, methoxy, methylcarbonylamino, nitro, propynyloxy, trifluoromethoxy, and trifluoromethyl;
R^{2′} is hydrogen or methyl;
R³ is selected from aminocarbonylmethyl; arylC₁-C₃alkyl, carboxymethyl, furylC₁-C₃alkyl, hydroxyC₁-C₃alkyl, HOS(O)₂C₁-C₃alkyl, CH₃S(O)₂NHC(O)(C₁-C₃alkyl), and tetrazolyl; wherein the aryl part of the arylC₁-C₃alkyl is optionally substituted with one, two, or three aminoC₁-C₃alkyl groups;
R⁴ is selected from arylC₁-C₂alkyl and heteroarylmethyl, and wherein the aryl part of the arylC₁-C₂alkyl and the heteroaryl part of the heteroarylmethyl are optionally substituted with one or more groups independently selected from amino, halo, hydroxy, methoxy, methyl, and trifluoromethyl;
R⁵ is selected from C₁-C₅alkyl, arylmethyl, C₃-C₆cycloalkyl, (C₃-C₆cycloalkyl)methyl, heteroarylmethyl, hydroxyC₂alkyl, methoxymethyl, and phenyl; wherein the aryl part of the arylmethyl is optionally substituted with one, two, three, four, or five groups independently selected from amino, aminocarbonyl, aminomethyl, aryl, arylmethoxy, aryloxy, carboxy, carboxymethoxy, (C₃-C₆cycloalkyl)oxy, cyano, halo, heteroaryl, hydroxy, and propynyloxy; wherein the aryl is further optionally substituted with C₁-C₃alkyl, C₁-C₃alkylcarbonylamino, carboxy, and hydroxy;
R⁶ is biphenylmethyl;
R⁷ is selected from C₁-C₅alkyl, aminoC₁-C₄alkyl, aminocarbonylethyl, aminocarbonylmethyl, arylmethyl, butynyl, carboxyethyl, C₃-C₆cycloalkyl, (C₃-C₆cycloalkyl)methyl, heteroarylmethyl, hydroxyC₂alkyl, methylcarbonylaminoC₂-C₄alkyl, phenyl, and NH₂C(X)NHC₂-C₄alkyl, wherein X is O or NH; wherein the aryl part of the arylmethyl is optionally substituted with one, two, or three groups independently selected from arylmethoxy, carboxy, carboxymethoxy, hydroxy, propynyloxy, and trifluoromethoxyl;
R⁸ is selected from C₁-C₄alkyl, C₁-C₄alkylcarbonylaminoC₁-C₄alkyl, aminoC₁-C₄alkyl, aminocarbonylC₁-C₂alkyl, (C₇H₁₅O₆)aminomethyl, arylmethyl, carboxyC₁-C₃alkyl, heteroarylmethyl, and hydroxymethyl; wherein the aryl part of the arylmethyl is optionally substituted with one, two, or three groups independently selected from aminomethyl and hydroxy;
R^{8′} is hydrogen;
R⁹ is selected from C₁-C₄alkyl, cyclohexylmethyl, and phenylmethyl; wherein the phenyl part of the phenylmethyl is optionally substituted with one or two hydroxy groups;
R¹⁰ is selected from aminoC₁-C₄alkyl, aminocarbonylC₁-C₂alkyl, (C₇H₁₅O₆)aminomethyl, butynyl, butylcarbonylaminoethyl; carboxyC₁-C₃alkyl, heteroarylmethyl, hydroxyC₁-C₂alkyl, and NH₂C(NH)NHmethyl;
R¹¹ is selected from C₄-C₈alkyl, arylC₁-C₂alkyl, C₃-C₆cycloalkylC₁-C₂alkyl, and heteroarylmethyl; wherein the aryl part of the arylC₁-C₂alkyl is optionally substituted with one, two, or three groups independently selected from aminomethyl, chloro, fluoro, hydroxy, methoxy, methyl, and trifluoromethyl;
R¹² is selected from C₃-C₄alkyl, carboxybutyl, hydroxyC₁-C₃alkyl, phenylmethyl, and propynyl; wherein the phenyl part of the phenylmethyl is optionally substituted with one, two, or three propynyloxy;
R¹³ is selected from C₃-C₄alkyl, C₁-C₄alkylcarbonylaminoC₂-C₄alkyl, aminoC₁-C₄alkyl, aminocarbonylC₁-C₂alkyl, arylmethyl, carboxyC₁-C₃alkyl, haloarylcarbonylaminopropyl; hydroxyC₁-C₃alkyl, heteroarylmethyl, and NH₂C(X)NHpropyl, wherein X is O or NH; wherein the aryl part of the arylmethyl is optionally substituted with one, two, or three groups independently selected from arylmethoxy and propynyloxy;
R¹⁴ is aminocarbonyl or —C(O)NR^{14′}CR¹⁵R^{15′}R^{15″}, wherein
R^{14′} is hydrogen or methyl;
R¹¹ is selected from hydrogen; C₁-C₂alkyl, C₁-C₄alkylcarbonylaminoC₁-C₃alkyl, aminoC₁-C₄alkyl, aminocarbonylC₁-C₂alkyl, arylmethyl, azidoC₂-C₃alkyl, carboxy; carboxyC₁-C₂alkyl, heteroarylmethyl, hydroxymethyl, propynyl, and NH₂C(NH)NHpropyl; wherein the aryl part of the arylmethyl is optionally substituted with one, two, or three groups independently selected from arylmethoxy and hydroxy;
R^{15′} is hydrogen or methyl; or R¹⁵ and R^{15′}, together with the atoms to which they are attached, form a cyclopropyl ring; and
R^{15″} is hydrogen, aminocarbonyl, carboxy, or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxy, carboxyethyl, heteroaryl, heteroarylethyl, hydroxymethyl, methoxymethyl, NH₂C(NH)NHpropyl, and propynyl; wherein the aryl part of the arylmethyl and the heteroaryl are optionally substituted with one or more groups independently selected from arylmethoxy and methyl; and
R^{16′} is hydrogen, aminocarbonyl, carboxy, methyl, or —(CH₂)ₘC(O)NHCHR¹⁷R^{17′}; wherein
m is 0, 1, or 2;
R¹⁷ is propynyl; and
R^{17′} is aminocarbonyl or carboxy; and
R^{a} is hydrogen or methyl; or R¹ and R^{a}, together with the atoms to which they are attached, form a pyrrolidine or piperazine ring, wherein each ring is optionally substituted with an amino group.

In some aspects, the present disclosure provides a compound of Formula (I), or a pharmaceutically acceptable salt thereof, wherein
R¹ is selected from C₁-C₄alkyl, C₁-C₄alkylcarbonylaminoC₂-C₄alkyl, aminoC₁-C₃alkyl, aminocarbonylC₁-C₂alkyl, carboxypropyl, cyanomethyl, heteroarylmethyl, heterocyclylmethyl, hydroxyC₂alkyl; methoxyC₁-C₂alkyl, methylaminoC₁-C₂alkyl, NH₂C(NH)NHpropyl, and H₂NC(NH)piperidinyl; arylC₁-C₂alkyl; wherein the aryl part of the arylC₁-C₂alkyl is optionally substituted with one, two, or three groups independently selected from aminocarbonyl, carboxy, carboxymethoxy, halo, methoxy, methylcarbonylamino, propynyloxy, and trifluoromethyl;
R^{1′} is hydrogen;
R² is selected from arylC₁-C₂alkyl and heteroarylC₁-C₂alkyl, wherein the aryl part of the arylC₁-C₂alkyl and the heteroaryl part of the heteroarylC₁-C₂alkyl are optionally substituted with one or more groups independently selected from amino, aminocarbonyl, aminomethyl, carboxy, carboxymethyl, carboxymethoxy, cyano, halo, hydroxy, methoxy, methyl, nitro, and propynyloxy;
R^{2′} is hydrogen or methyl;
R³ is selected from aminocarbonylmethyl, carboxymethyl, and tetrazolyl;
R⁴ is selected from arylmethyl and heteroarylmethyl; wherein the aryl part of the arylmethyl and the heteroaryl part of the heteroarylmethyl are optionally substituted with one, two, three, four, or five groups independently selected from amino, halo, hydroxy, methoxy, methyl, and trifluoromethyl;
R⁵ is selected from C₁-C₅alkyl, arylmethyl, C₃-C₆cycloalkyl, (C₃-C₆cycloalkyl)methyl, hydroxyC₂alkyl, methoxymethyl, and phenyl; wherein the aryl part of the arylmethyl is optionally substituted with one, two, three, four, or five groups independently selected from aminomethyl, aminocarbonyl, carboxy, carboxymethoxy, hydroxy, and propynyloxy;
R⁶ is biphenylmethyl;
R⁷ is selected from C₁-C₄alkyl, aminoC₁-C₄alkyl, aminocarbonylethyl, aminocarbonylmethyl, arylmethyl, butynyl, carboxyethyl, C₃-C₆cycloalkyl, heteroarylmethyl, hydroxyC₂alkyl, methylcarbonylaminobutyl, phenyl, and NH₂C(X)NHC₂-C₄alkyl, wherein X is O or NH; wherein the aryl part of the arylmethyl is optionally substituted with one, two, or three groups independently selected from carboxy, carboxymethoxy, hydroxy, propynyloxy, and trifluoromethoxyl;
R⁸ is selected from C₁-C₄alkyl, C₁-C₄alkylcarbonylaminoC₂-C₄alkyl, aminoC₁-C₄alkyl, aminocarbonylethyl, carboxypropyl, hydroxymethyl, and imidazolylmethyl;
R^{8′} is hydrogen;
R⁹ is selected from C₁-C₄alkyl, cyclohexylmethyl, and phenylmethyl; wherein the phenyl part of the phenylmethyl is optionally substituted with one or two hydroxy groups;
R¹⁰ is selected from aminoC₁-C₄alkyl, aminocarbonylC₁-C₂alkyl, butylcarbonylaminoethyl, butynyl, carboxyC₁-C₃alkyl, hydroxyC₁-C₂alkyl, imidazolylmethyl, and NH₂C(NH)NHmethyl;
R¹¹ is selected from butyl, cyclohexylmethyl, and phenylmethyl; wherein
the phenyl part of the phenylmethyl is optionally substituted with one, two, or three groups independently selected from fluoro and methyl;
R¹² is selected from C₃-C₄alkyl, carboxybutyl, hydroxyC₁-C₃alkyl, and phenylmethyl; wherein the phenyl part of the phenylmethyl is optionally substituted with one, two, or three propynyloxy groups;
R¹³ is selected from aminoC₁-C₄alkyl, C₁-C₄alkylcarbonylaminoC₂-C₄alkyl, aminocarbonylC₁-C₂alkyl, arylmethyl. butyl, carboxyC₁-C₂alkyl, heteroarylmethyl, hydroxyC₁-C₃alkyl, and NH₂C(X)NHpropyl, wherein X is O or NH; wherein the aryl part of the arylmethyl is optionally substituted with one, two, or three propynyloxy groups;
R¹⁴ is aminocarbonyl or —C(O)NR^{14′}CR¹⁵R^{15′}R^{15″}, wherein
R^{14′} is hydrogen or methyl;
R¹⁵ is selected from hydrogen, C₁-C₂alkyl, aminoC₁-C₄alkyl, aminocarbonylmethyl, butylcarbonylaminoethyl, carboxy, carboxyethyl, hydroxymethyl, NH₂C(NH)NHpropyl, and propynyl;
R^{15′} is hydrogen; methyl; or R¹⁵ and R^{15′}, together with the atoms to which they are attached, form a cyclopropyl ring; and
R^{15″} is hydrogen, aminocarbonyl, carboxy, or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxyethyl, heteroaryl, heteroarylethyl, hydroxymethyl, methoxymethyl, NH₂C(NH)NHpropyl, and propynyl; wherein the aryl part of the arylmethyl and the heteroaryl are optionally substituted with one or more groups independently selected from arylmethoxy and methyl;
R^{16′} is hydrogen, aminocarbonyl, carboxy, methyl, or —(CH₂)ₘC(O)NHCHR¹⁷R^{17′}; wherein
m is 0, 1, or 2; wherein
R¹⁷ is propynyl; and
R^{17′} is aminocarbonyl; and
R^{a} is hydrogen or methyl; or R¹ and R^{a}, together with the atoms to which they are attached, form a pyrrolidine ring, wherein the pyrrolidine ring is optionally substituted with an amino group.

In some aspects, the present disclosure provides a compound of Formula (I), or a pharmaceutically acceptable salt thereof, wherein
R¹ is selected from C₂-C₄alkyl, aminoC₁-C₂alkyl, aminocarbonylmethyl, heteroarylmethyl, hydroxyC₂alkyl, morpholinylmethyl, NH₂C(NH)NHpropyl, and phenylmethyl; wherein the phenyl part of the phenylmethyl is optionally substituted with one, two, or three groups independently selected from carboxymethoxy and fluoro;
R^{1′} is hydrogen;
R² is selected from phenylmethyl and pyridylmethyl, and wherein
the phenyl part of the phenylmethyl are optionally substituted with one, two, or three groups independently selected from hydroxy, carboxy, and carboxymethoxy;
R^{2′} is hydrogen;
R³ is carboxymethyl;
R⁴ is selected from indolylmethyl and phenylmethyl; wherein the phenyl part of the phenylmethyl is optionally substituted with one, two, or three groups independently selected from hydroxy and methyl;
R⁵ is selected from phenylmethyl and propyl; wherein the phenyl part of the phenylmethyl is optionally substituted with one, two, or three groups independently selected from carboxy, carboxymethoxy, hydroxy, and propynyloxy; and
R⁶ is biphenylmethyl;
R⁷ is selected from C₃-C₄alkyl, NH₂C(O)NHpropyl, and phenylmethyl; wherein the phenyl part of the phenylmethyl is optionally substituted with one, two, or three groups independently selected from carboxy, carboxymethoxy, hydroxy, and propynyloxy;
R⁸ is selected from aminopropyl and methyl;
R^{8′} is hydrogen;
R⁹ is isobutyl;
R¹⁰ is aminoethyl;
R¹¹ is selected from butyl and cyclohexylmethyl;
R¹² is selected from hydroxyisopropyl, hydroxypropyl, isopropyl, and propyl;
R¹³ is selected from aminopropyl, carboxyethyl, hydroxyC₁-C₂alkyl, imidazolylmethyl, and phenylmethyl; wherein the phenyl part of the phenylmethyl is optionally substituted with one, two, or three propynyloxy groups;
R¹⁴ is aminocarbonyl or —C(O)NR^{14′}CR¹⁵R^{15′}R^{15″}, wherein
R^{14′} is hydrogen;
R¹⁵ is selected from aminocarbonylmethyl, aminoethyl, and methyl;
R^{15′} is hydrogen; and
R^{15″} is hydrogen, aminocarbonyl, or —(CH₂)ₙC(O)NHCHR¹⁶R^{16′}; wherein
n is 0 or 1;
R¹⁶ is propynyl; and
R^{16′} is hydrogen, aminocarbonyl, or carboxy; and
R^{a} is hydrogen.

In some aspects, the present disclosure provides a pharmaceutical composition comprising a compound of Formula (I), or a pharmaceutically acceptable salt thereof.

In some aspects, the present disclosure provides a method of enhancing, stimulating, and/or increasing an immune response in a subject in need thereof, wherein the method comprises administering to the subject a therapeutically effective amount of a compound of Formula (I), or a pharmaceutically acceptable salt thereof.

In some aspects, the present disclosure provides a method of blocking the interaction of PD-1 with PD-L1 in a subject, wherein the method comprises administering to the subject a therapeutically effective amount of a compound of Formula (I), or a pharmaceutically acceptable salt thereof.

### DETAILED DESCRIPTION

### Definitions

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₁-C₆alkoxy,” as used herein, refers to a C₁-C₆alkyl group attached to the parent molecular moiety through an oxygen atom.

The term “C₁-C₆alkoxyC₁-C₆alkyl,” as used herein, refers to a C₁-C₆alkoxy group attached to the parent molecular moiety through a C₁-C₆alkyl group.

The term “alkyl,” as used herein, refers to a group derived from a straight or branched chain saturated hydrocarbon containing carbon atoms. The term “alkyl” may be proceeded by “C_{#}-C_{#}” wherein the # is an integer and refers to the number of carbon atoms. For example, C₁-C₂alkyl contains one to two carbon atoms and C₁-C₃alkyl contains one to three carbon atoms.

The term “C₁-C₆alkylC₂-C₆alkynyl,” as used herein, refers to a C₁-C₆alkyl group attached to the parent molecular moiety through a C₂-C₆alkynyl group.

The term “C₁-C₆alkylamino,” as used herein, refers to a group having the formula —NH, wherein R is a C₁-C₆alkyl group.

The term “C₁-C₆alkylaminoC₁-C₆alkyl,” as used herein, refers to a C₁-C₆alkylamino group attached to the parent molecular moiety through a C₁-C₆alkyl group.

The term “C₁-C₃alkylcarbonyl,” as used herein, refers to a C₁-C₃alkyl group attached to the parent molecular moiety through a carbonyl group.

The term “C₁-C₆alkylcarbonyl,” as used herein, refers to a C₁-C₆alkyl group attached to the parent molecular moiety through a carbonyl group.

The term “C₁-C₆alkylcarbonylamino,” as used herein, refers to —NHC(O)R^{a}, wherein R^{a} is a C₁-C₆alkyl group.

The term “C₁-C₆alkylcarbonylaminoC₁-C₆alkyl,” as used herein, refers to a C₁-C₆alkylcarbonylamino group attached to the parent molecular moiety through a C₁-C₆alkyl group.

The term “C₁-C₄alkylcarbonylaminoC₁-C₃alkyl,” as used herein, refers to a C₁-C₄alkylcarbonylamino group attached to the parent molecular moiety through a C₁-C₃alkyl group.

The term “C₁-C₄alkylcarbonylaminoC₁-C₄alkyl,” as used herein, refers to a C₁-C₄alkylcarbonylamino group attached to the parent molecular moiety through a C₁-C₄alkyl group.

The term “C₁-C₄alkylcarbonylaminoC₂-C₄alkyl,” as used herein, refers to a C₁-C₄alkylcarbonylamino group attached to the parent molecular moiety through a C₂-C₄alkyl group.

The term “C₂-C₄alkynyl,” as used herein, refers to a group derived from a straight or branched chain hydrocarbon containing one or more carbon-carbon triple bonds containing two to four carbon atoms.

The term “C₂-C₆alkynyl,” as used herein, refers to a group derived from a straight or branched chain hydrocarbon containing one or more carbon-carbon triple bonds containing two to six carbon atoms.

The term “C₂-C₆alkynyloxy,” as used herein, refers to a C₂-C₆alkynyl group attached to the parent molecular moiety through an oxygen atom.

The term “amino,” as used herein, refers to —NH₂.

The term “aminoC₁-C₆alkoxy,” as used herein, refers to an amino group attached to the parent molecular moiety through a C₁-C₆alkoxy group.

The term “aminoC₁-C₂alkyl,” as used herein, refers to an amino group attached to the parent molecular moiety through a C₁-C₂alkyl group.

The term “aminoC₁-C₃alkyl,” as used herein, refers to an amino group attached to the parent molecular moiety through a C₁-C₃alkyl group.

The term “aminoC₁-C₄alkyl,” as used herein, refers to an amino group attached to the parent molecular moiety through a C₁-C₄alkyl group.

The term “aminoC₁-C₆alkyl,” as used herein, refers to an amino group attached to the parent molecular moiety through a C₁-C₆alkyl group.

The term “aminocarbonyl,” as used herein, refers to an amino group attached to the parent molecular moiety through a carbonyl group.

The term “aminocarbonylC₁-C₂alkyl,” as used herein, refers to an aminocarbonyl group attached to the parent molecular moiety through a C₁-C₂alkyl group.

The term “aminocarbonylC₁-C₆alkyl,” as used herein, refers to an aminocarbonyl group attached to the parent molecular moiety through a C₁-C₆alkyl group.

The term “aminocarbonylethyl,” as used herein, refers to an aminocarbonyl group attached to the parent molecular moiety through a —CH₂CH₂— group.

The term “aminocarbonylmethyl,” as used herein, refers to an aminocarbonyl group attached to the parent molecular moiety through a —CH₂— group.

The term “aminoethoxy,” as used herein, refers to a amino group attached to the parent molecular moiety though an ethoxy group.

The term “aminoethyl,” as used herein, refers to a amino group attached to the parent molecular moiety though an ethyl group.

The term “aminomethyl,” as used herein, refers to an amino group attached to the parent molecular moiety through a —CH₂— group.

The term “aminopropyl,” as used herein, refers to a amino group attached to the parent molecular moiety though a propyl group.

The term “C₃-C₈cycloalkyl”, as used herein, refers to a saturated monocyclic or bicyclic hydrocarbon ring system having three to eight carbon atoms and zero heteroatoms. The bicyclic rings can be fused, spirocyclic, or bridged. Representative examples of cycloalkyl groups include, but are not limited to, cyclopropyl, cyclopentyl, octahydropentalene, and bicyclo[3.1.1]heptyl.

The term “C₃-C₆cycloalkyl”, as used herein, refers to a saturated monocyclic hydrocarbon ring system having three to six carbon atoms and zero heteroatoms. Representative examples of cycloalkyl groups include, but are not limited to, cyclopropyl, cyclopentyl, and cyclohexyl.

The term “C₃-C₆cycloalkylC₁-C₂alkyl,” as used herein, refers to a C₃-C₆cycloalkyl group attached to the parent molecular moiety through a C₁-C₂alkyl group.

The term “C₃-C₆cycloalkylC₁-C₃alkyl,” as used herein, refers to a C₃-C₆cycloalkyl group attached to the parent molecular moiety through a C₁-C₃alkyl group.

The term “(C₃-C₈cycloalkyl)C₁-C₆alkyl,” as used herein, refers to a C₃-C₈cycloalkyl group attached to the parent molecular moiety through a C₁-C₆alkyl group.

The term “(C₃-C₆cycloalkyl)methyl,” as used herein, refers to a C₃-C₆cycloalkyl group attached to the parent molecular moiety through a —CH₂— group.

The term “(C₃-C₆cycloalkyl)oxy,” as used herein, refers to a C₃-C₆cycloalkyl group attached to the parent molecular moiety through an oxygen atom.

The term “(C₇H₁₅O₆)amino,” as used herein, refers to a group having the formula C₇H₁₅O₆ that is attached to the parent molecular moiety through an amino group.

The term “(C₇H₁₅O₆)aminoC₁-C₆alkyl,” as used herein, refers to a (C₇H₁₅O₆)amino group attached to the parent molecular moiety through a C₁-C₆alkyl group.

The term “(C₇H₁₅O₆)aminomethyl,” as used herein, refers to a (C₇H₁₅O₆)amino group attached to the parent molecular moiety through a —CH₂— group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “arylC₁-C₆alkoxy,” as used herein, refers to an aryl group attached to the parent molecular moiety through a C₁-C₆alkoxy group.

The term “arylC₁-C₆alkoxyC₁-C₆alkyl,” as used herein, refers to an arylC₁-C₆alkoxy group attached to the parent molecular moiety through a C₁-C₆alkyl group.

The term “arylC₁-C₆alkyl,” as used herein, refers to an aryl group attached to the parent molecular moiety through a C₁-C₆alkyl group.

The term “arylC₁-C₂alkyl,” as used herein, refers to an aryl group attached to the parent molecular moiety through a C₁-C₂alkyl group.

The term “arylC₁-C₃alkyl,” as used herein, refers to an aryl group attached to the parent molecular moiety through a C₁-C₃alkyl group.

The term “aryl-aryl,” as used herein, refers to an aryl group attached to the parent molecular moiety through a second aryl group.

The term “aryl-arylC₁-C₃alkyl,” as used herein, refers to an aryl-aryl group attached to the parent molecular moiety through a C₁-C₃alkyl group.

The term “arylcarbonyl,” as used herein, refers to an aryl group attached to the parent molecular moiety through a carbonyl group.

The term “arylcarbonylamino,” as used herein, refers to a group of formula —NHC(O)R, wherein R is an aryl group.

The term “arylcarbonylaminoC₁-C₆alkyl,” as used herein, refers to an arylcarbonylamino group attached to the parent molecular moeity though a C₁-C₆alkyl.

The term “arylcarbonylaminoC₁-C₂alkyl,” as used herein, refers to an arylcarbonylamino group attached to the parent molecular moiety through a C₁-C₂alkyl group.

The term “aryl-heteroaryl,” as used herein, refers to an aryl group attached to the parent molecular moiety through a heteroaryl group.

The term “aryl-heteroarylC₁-C₃alkyl,” as used herein, refers to a aryl-heteroaryl group attached to the parent molecular moiety through a C₁-C₃alkyl group.

The term “arylmethoxy,” as used herein, refers to an aryl group attached to the parent molecular moiety though a methoxy group.

The term “arylmethoxymethyl,” as used herein, refers to an arylmethoxy group attached to the parent molecular moiety through a —CH₂— group.

The term “arylmethyl,” as used herein, refers to an aryl group attached to the parent molecular moiety through a —CH₂— group.

The term “aryloxy,” as used herein, refers to an aryl group attached to the parent molecular moiety through an oxygen atom.

The term “azido,” as used herein, refers to —N₃.

The term “azidoC₁-C₂alkyl,” as used herein, refers to an azido group attached to the parent molecular moiety through a C₁-C₂alkyl group.

The term “azidoC₁-C₆alkyl,” as used herein, refers to an azido group attached to the parent molecular moiety through a C₁-C₆alkyl group.

The term “azidoC₂-C₃alkyl,” as used herein, refers to an azido group attached to the parent molecular moiety through a C₂-C₃alkyl group.

The term “azidoC₂-C₄alkyl,” as used herein, refers to an azido group attached to the parent molecular moiety through a C₂-C₄alkyl group.

The term “biphenylC₁-C₆alkyl,” as used herein, refers to a biphenyl group attached to the parent molecular moiety through a C₁-C₆alkyl group. The biphenyl group can be attached to the alkyl moiety through any substitutable atom in the group.

The term “biphenylmethyl,” as used herein refers to a biphenyl group attached to the parent molecular moiety through a —CH₂— group.

The term “biscarboxyCHC₁-C₆alkyl,” as used herein, refers (CO₂H)₂CH(C₁-C₆alkyl)-.

The term “biscarboxyethyl,” as used herein, refers to (CO₂H)₂CHCH₂—.

The term “butylcarbonylamino,” as used herein, refers to —NHC(O)R^{a}, wherein R^{a} is butyl.

The term “butylcarbonylaminoethyl,” as used herein, refers to a butylcarbonylamino group attached to the parent molecular moiety through a —CH₂CH₂— group.

The term “carbonyl,” as used herein, refers to —C(O)—.

The term “carboxy,” as used herein, refers to —CO₂H.

The term “carboxyC₁-C₆alkoxy,” as used herein, refers to a carboxy group attached to the parent molecular moiety through a C₁-C₆alkoxy group.

The term “carboxyC₁-C₂alkyl,” as used herein, refers to a carboxy group attached to the parent molecular moiety through a C₁-C₂alkyl group.

The term “carboxyC₁-C₃alkyl,” as used herein, refers to a carboxy group attached to the parent molecular moiety through a C₁-C₃alkyl group.

The term “carboxyC₁-C₆alkyl,” as used herein, refers to a carboxy group attached to the parent molecular moiety through a C₁-C₆alkyl group.

The term “carboxybutyl,” as used herein, refers to a carboxy group attached to the parent molecular moiety though a butyl group.

The term “carboxyethyl,” as used herein, refers to refers to a carboxy group attached to the parent molecular moiety though a —CH₂CH₂— group.

The term “carboxymethoxy,” as used herein, refers to a carboxy group attached to the parent molecular moiety though a methoxy group.

The term “carboxymethyl,” as used herein, refers to refers to a carboxy group attached to the parent molecular moiety through a —CH₂— group.

The term “carboxyphenyl,” as used herein, refers to a carboxy group attached to the parent molecular moiety through a phenyl group.

The term “carboxypropyl,” as used herein, refers to a carboxy group attached to the parent molecular moiety though a propyl group.

The term “cyano,” as used herein, refers to —CN.

The term “cyanoC₁-C₆alkyl,” as used herein, refers to a cyano group attached to the parent molecular moiety though a C₁-C₆alkyl.

The term “cyanomethyl,” as used herein, refers to a cyano group attached to the parent molecular moiety through a —CH₂— group.

The term “cyclohexylmethyl,” as used herein, refers to a cyclohexyl group attached to the parent molecular moiety through a —CH₂— group.

The term “ethoxy,” as used herein, refers to —OCH₂CH₃.

The term “fluoroC₁-C₆alkyl,” as used herein, refers to a C₁-C₆alkyl group substituted with one, two, three, four, five, or six fluoro groups.

The term “furylC₁-C₃alkyl,” as used herein, refers to a furyl group attached to the parent molecular moiety through a C₁-C₃alkyl group.

The term “furylmethyl,” as used herein, refers to a furyl group attached to the parent molecular moiety through a —CH₂— group.

The terms “halo” and “halogen,” as used herein, refer to F, Cl, Br, or I.

The term “haloC₁-C₆alkoxy,” as used herein, refers to a C₁-C₆alkoxy group substituted with one, two, three, four, five, or six halogen atoms.

The term “haloaryl,” as used herein, refers to an aryl group substituted with one, two, three, four, or five halo groups.

The term “haloarylcarbonyl,” as used herein, refers to a haloaryl group attached to the parent molecular moiety through a carbonyl group.

The term “haloarylcarbonylamino,” as used herein, refers to a haloarylcarbonyl group attached to the parent molecular moiety though an amino group.

The term “haloarylcarbonylaminoC₁-C₆alkyl,” as used herein, refers to a haloarylcarbonylamino group attached to the parent molecular moiety through a C₁-C₆alkyl group.

The term “haloarylcarbonylaminopropyl,” as used herein, refers to a haloarylcarbonylamino group attached to the parent molecular moiety through a propyl group.

The term “heteroaryl,” as used herein, refers to a monocyclic, bicyclic, and tricyclic ring system having a total of five to fourteen ring members, wherein said ring system has a single point of attachment to the rest of the molecule, at least one ring in the system is aromatic, at least one ring in the system contains one or more heteroatoms independently selected from nitrogen, oxygen, sulfur or phosphorus, and wherein each ring in the system contains 4 to 7 ring members. The term “heteroaryl” may be used interchangeably with the term “heteroaryl ring” or the term “heteroaromatic.”

The term “heteroarylC₁-C₂alkyl,” as used herein, refers to a heteroaryl group attached to the parent molecular moiety though a C₁-C₂alkyl group.

The term “heteroarylC₁-C₆alkyl,” as used herein, refers to a heteroaryl group attached to the parent molecular moiety though a C₁-C₆alkyl group.

The term “heteroaryl-aryl,” as used herein, refers to a heteroaryl group attached to the parent molecular moiety through an aryl group.

The term “heteroaryl-arylC₁-C₃alkyl,” as used herein, refers to a heteroaryl-aryl group attached to the parent molecular moiety through a C₁-C₃alkyl group.

The term “heteroarylethyl,” as used herein, refers to a heteroaryl group attached to the parent molecular moiety though a —CH₂CH₂— group.

The term “heteroaryl-heteroaryl,” as used herein, refers to a heteroaryl group attached to the parent molecular moiety through a heteroaryl group.

The term “heteroaryl-heteroarylC₁-C₃alkyl,” as used herein, refers to a heteroaryl-heteroaryl group attached to the parent molecular moiety through a C₁-C₃alkyl group.

The term “heteroarylmethyl,” as used herein, refers to a heteroaryl group attached to the parent molecular moiety through a —CH₂— group.

The term “heteroarylpropyl,” as used herein, refers to a heteroaryl group attached to the parent molecular moiety though a propyl group.

The term “heterocyclyl,” as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure can be attached to the parent molecular moiety through any substitutable atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylC₁-C₆alkyl,” as used herein, refers to a heterocyclyl attached to the parent molecular moiety through a C₁-C₆alkyl group.

The term “heterocyclylmethyl,” as used herein, refers to a heterocyclyl group attached to the parent molecular moiety through a —CH₂— group.

The term “hydroxy,” as used herein, refers to —OH.

The term “hydroxyC₁-C₂alkyl,” as used herein, refers to a hydroxyl group attached to the parent molecular moiety though a C₁-C₂alkyl group.

The term “hydroxyC₁-C₃alkyl,” as used herein, refers to a hydroxyl group attached to the parent molecular moiety though a C₁-C₃alkyl group.

The term “hydroxyC₁-C₄alkyl,” as used herein, refers to a hydroxyl group attached to the parent molecular moiety though a C₁-C₄alkyl group.

The term “hydroxyC₁-C₆alkyl,” as used herein, refers to a hydroxyl group attached to the parent molecular moiety though a C₁-C₆alkyl group.

The term “hydroxyC₂alkyl,” as used herein, refers to a hydroxyl group attached to the parent molecular moiety though a —CH₂CH₂— group.

The term “hydroxyC₂-C₃alkyl,” as used herein, refers to a hydroxyl group attached to the parent molecular moiety though a C₂-C₃alkyl group.

The term “hydroxyisopropyl,” as used herein, refers to a hydroxyl group attached to the parent molecular moiety though an isopropyl.

The term “hydroxymethyl,” as used herein, refers to a hydroxyl group attached to the parent molecular moiety through a —CH₂— group.

The term “hydroxypropyl,” as used herein, refers to a hydroxyl group attached to the parent molecular moiety though a propyl.

The term “imidazolylmethyl,” as used herein, refers to an imidazolyl group attached to the parent molecular moiety through a —CH₂— group.

The term “indolylmethyl,” as used herein, refers to an indolyl group attached to the parent molecular moiety through a —CH₂— group.

The term “methoxy,” as used herein, refers to —OCH₃.

The term “methoxyC₁-C₂alkyl,” as used herein, refers to a methoxy group attached to the parent molecular moiety though a C₁-C₂alkyl group.

The term “methoxymethyl, as used herein, refers to a methoxy group attached to the parent molecular moiety though a methyl group.

The term “methylaminoC₁-C₂alkyl,” as used herein, refers to a methylamino group attached to the parent molecular moiety through a C₁-C₂alkyl group.

The term “methylcarbonylamino,” as used herein, refers to CH₃C(O)NH—.

The term “methylcarbonylamino,” as used herein, refers to a methylcarbonyl group attached to the parent molecular moiety through an amino group.

The term “methylcarbonylaminobutyl,” as used herein, refers to a methylcarbonylamino group attached to the parent molecular moiety through a butyl group.

The term “methylcarbonylaminoC₂-C₄alkyl,” as used herein, refers to a methylcarbonylamino group attached to the parent molecular moiety through a C₂-C₄alkyl group.

The term “morpholinylmethyl,” as used herein, refers to a morpholinyl group attached to the parent molecular moiety through a —CH₂— group.

The term “nitro,” as used herein, refers to —NO₂.

The term “phenylmethyl,” as used herein, refers to a phenyl group attached to the parent molecular moiety through a —CH₂— group.

The term “propynyl,” as used herein, refers to —CCCH₃.

The term “propynyloxy,” as used herein, refers to —OC≡CCH₃.

The term “pyridinylmethyl,” as used herein, refers to a pyridinyl group attached to the parent molecular moiety through a —CH₂— group.

The term “tetrazolylmethyl,” as used herein, refers to a tetrazolyl group attached to the parent molecular moiety through a —CH₂— group.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, and benign prostatic hypertrophy).

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1,” “Programmed Cell Death Ligand 1,” “PD-L1,” “PDL1,” “hPD-L1,” “hPD-L1,” and “B7-H1” are used interchangeably, and include variants, isoforms, species homologs of human PD-L1, and analogs having at least one common epitope with PD-LI. The complete PD-L1 sequence can be found under GENBANK® Accession No. NP_054862.

The terms “Programmed Death 1,” “Programmed Cell Death 1,” “Protein PD-1,” “PD-1,” “PD1,” “hPD-1” and “hPD-I” are used interchangeably, and include variants, isoforms, species homologs of human PD-1, and analogs having at least one common epitope with PD-1. The complete PD-I sequence can be found under GENBANK® Accession No. U64863.

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Various aspect of the disclosure are described in greater detail below.

### Compounds of Formula (I)

In an aspect, the present disclosure provides a compound of Formula (I):

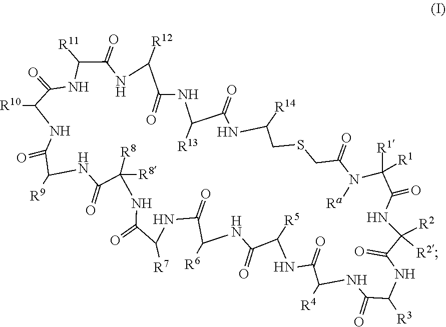

or a pharmaceutically acceptable salt thereof, wherein:
R¹ is selected from C₁-C₆alkoxyC₁-C₆alkyl; C₁-C₆alkyl; C₁-C₆alkylaminoC₁-C₆alkyl; C₁-C₆alkylcarbonylaminoC₁-C₆alkyl; aminoC₁-C₆alkyl; aminocarbonylC₁-C₆alkyl; arylC₁-C₆alkyl; arylcarbonylaminoC₁-C₆alkyl; carboxyC₁-C₆alkyl; cyanoC₁-C₆alkyl; heteroarylC₁-C₆alkyl; heterocyclylC₁-C₆alkyl; hydroxyC₁-C₆alkyl; NH₂C(X)NHC₁-C₆alkyl, wherein X is O or NH; and

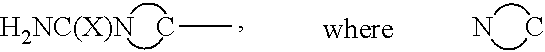

represents an azetidine, piperidine, or pyrrolidine ring; wherein the aryl part of the arylC₁-C₆alkyl and the arylcarbonylaminoC₁-C₆alkyl and the heteroaryl part of the heteroarylC₁-C₆alkyl are optionally substituted with one, two, three, four, or five groups independently selected from C₁-C₆alkoxy, C₁-C₆alkylcarbonylamino, C₂-C₆alkynyloxy, aminocarbonyl, aryl optionally substituted with one or two carboxy groups, arylC₁-C₆alkoxy, carboxy, carboxyC₁-C₆alkoxy, halo, and trifluoromethyl;
R^{1′} is hydrogen or C₁-C₆alkyl;
R² is selected from C₁-C₆alkoxyC₁-C₆alkyl; arylC₁-C₆alkyl; azidoC₁-C₆alkyl; biscarboxyCHC₁-C₆alkyl; carboxyC₁-C₆alkyl; and heteroarylC₁-C₆alkyl; wherein
the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, C₁-C₆alkylcarbonylamino, C₂-C₆alkynyloxy, amino, aminoC₁-C₆alkyl, aminocarbonyl, aryl optionally substituted with one or two carboxy groups, arylC₁-C₆alkoxy, arylcarbonyl, azido, carboxy, carboxyC₁-C₆alkoxy, carboxyC₁-C₆alkyl, cyano, halo, haloC₁-C₆alkoxy, hydroxy, nitro, and trifluoromethyl;
R^{2′} is hydrogen or C₁-C₆alkyl;
R³ is selected from C₁-C₆alkoxyC₁-C₆alkyl; aminocarbonylC₁-C₆alkyl, arylC₁-C₆alkoxyC₁-C₆alkyl, arylC₁-C₃alkyl, carboxyC₁-C₆alkyl, furylC₁-C₃alkyl, hydroxyC₁-C₆alkyl, HOS(O)₂C₁-C₃alkyl, CH₃S(O)₂NHC(O)(C₁-C₃alkyl), and tetrazolylC₁-C₃alkyl; wherein the aryl part of the arylC₁-C₃alkyl is optionally substituted with one, two, or three, aminoC₁-C₃alkyl groups;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one or more groups independently selected from C₁-C₆alkoxy, C₁-C₆alkyl, amino, cyano, C₁-C₆fluoroalkyl, halo, and hydroxy;
R⁵ is selected from C₁-C₆alkoxyC₁-C₆alkyl; C₁-C₆alkyl; aryl; arylC₁-C₆alkyl; cyanoC₁-C₆alkyl; C₃-C₈cycloalkyl; (C₃-C₈cycloalkyl)C₁-C₆alkyl; fluoroC₁-C₆alkyl; heteroarylC₁-C₆alkyl; and hydroxyC₁-C₆alkyl; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, amino, aminoC₁-C₆alkyl, aminocarbonyl, aryl, arylC₁-C₆alkoxy, aryloxy, carboxyC₁-C₆alkoxy, cyano, (C₃-C₆cycloalkyl)oxy, carboxy, halo, heteroaryl, and hydroxy, wherein the aryl is further optionally substituted with one, two, or three groups independently selected from C₁-C₃alkyl, C₁-C₃alkylcarbonylamino, carboxy, and hydroxy;
R⁶ is selected from aryl-arylC₁-C₃alkyl, aryl-heteroarylC₁-C₃alkyl, heteroaryl-arylC₁-C₃alkyl, and heteroaryl-heteroarylC₁-C₃alkyl, wherein each aryl and each heteroaryl are optionally substituted with one or more groups independently selected from C₁-C₆alkoxy, C₁-C₆alkyl, amino, cyano, C₁-C₆fluoroalkyl, halo, and hydroxyl;
R⁷ is selected from hydrogen; C₁-C₆alkyl; C₁-C₆alkylcarbonylaminoC₁-C₆alkyl; C₂-C₆alkynyl; aminoC₁-C₆alkyl; aminocarbonylC₁-C₆alkyl; aryl; arylC₁-C₆alkyl; carboxyC₁-C₆alkyl; C₃-C₈cycloalkyl; (C₃-C₈cycloalkyl)C₁-C₆alkyl; haloarylcarbonylaminoC₁-C₆alkyl; heteroarylC₁-C₆alkyl; hydroxyC₁-C₆alkyl; and NH₂C(X)NHC₁-C₆alkyl, where X is O or NH; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, arylC₁-C₆alkoxy, carboxy, carboxyC₁-C₆alkoxy, haloC₁-C₆alkoxy, and hydroxy;
R⁸ is selected from C₁-C₆alkyl; C₁-C₆alkylcarbonylaminoC₁-C₆alkyl; aminoC₁-C₆alkyl; (C₇H₁₅O₆)aminoC₁-C₆alkyl; aminocarbonylC₁-C₆alkyl; arylC₁-C₆alkyl; carboxyC₁-C₆alkyl; heterocyclyl; heteroarylC₁-C₆alkyl; and hydroxyC₁-C₆alkyl; wherein the aryl part of the arylC₁-C₆alkyl and the arylcarbonylaminoC₁-C₆alkyl are optionally substituted with one, two, three, four, or five groups independently selected from aminoC₁-C₆alkyl, halo, and hydroxy;
R^{8′} is hydrogen or R⁸ and R^{8′}, together with the atoms to which they are attached, form a C₃-C₈cycloalkyl ring;
R⁹ is selected from C₁-C₆alkyl; arylC₁-C₆alkyl; and C₃-C₈cycloalkylC₁-C₆alkyl; wherein the aryl part of the arylC₁-C₆alkyl is optionally substituted with one, two, three, four, or five groups independently selected from halo and hydroxy;
R¹⁰ is selected from C₁-C₆alkyl; C₂-C₆alkynyl; aminoC₁-C₆alkyl; aminocarbonylC₁-C₆alkyl; arylC₁-C₆alkyl; carboxyC₁-C₆alkyl; hydroxyC₁-C₆alkyl; (C₇H₁₅O₆)aminoC₁-C₆alkyl; C₁-C₆alkylcarbonylaminoC₁-C₆alkyl; heteroarylC₁-C₆alkyl; and NH₂C(X)NHC₁-C₆alkyl, wherein X is O or NH; wherein the aryl part of the arylC₁-C₆alkyl is optionally substituted with one, two, three, four, or five groups independently selected from C₂-C₆alkynyloxy, and arylC₁-C₆alkoxy;
R¹¹ is selected from C₁-C₈alkyl; arylC₁-C₆alkyl; C₃-C₈cycloalkylC₁-C₆alkyl; and heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₁-C₆alkoxy, aminoC₁-C₆alkyl cyano, halo, hydroxy, and trifluoromethyl;
R¹² is selected from C₁-C₆alkyl, C₂-C₆alkynyl, arylC₁-C₆alkyl, carboxyC₁-C₆alkyl, and hydroxyC₁-C₆alkyl; wherein the aryl part of the arylC₁-C₆alkyl is optionally substituted with one, two, three, four, or five groups independently selected from C₂-C₆alkynyloxy and arylC₁-C₆alkoxy;
R¹³ is selected from C₁-C₆alkyl, C₁-C₆alkylcarbonylaminoC₁-C₆alkyl, aminoC₁-C₆alkyl, aminocarbonylC₁-C₆alkyl, arylC₁-C₆alkyl, carboxyC₁-C₆alkyl, haloarylcarbonylaminoC₁-C₆alkyl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl is optionally substituted with one, two, three, four, or five groups independently selected from C₂-C₆alkynyloxy and arylC₁-C₆alkoxy;
R¹⁴ is aminocarbonyl; carboxy; or —C(O)NR^{14′}CR¹⁵R^{15′}R^{15″}, wherein
R^{14′} is hydrogen or C₁-C₆alkyl, or R¹⁵ and R^{14′}, together with the atoms to which they are attached, form an azetidine, morpholine, piperazine, piperidine, or pyrrolidine ring, wherein each ring is optionally substituted with an amino or a hydroxy group;
R¹⁵ is selected from hydrogen, C₁-C₆alkyl, C₁-C₆alkylcarbonylaminoC₁-C₆alkyl, C₂-C₆alkynyl, aminoC₁-C₆alkyl, aminocarbonylC₁-C₆alkyl, arylC₁-C₆alkyl, azidoC₁-C₆alkyl, carboxy, carboxyC₁-C₆alkyl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl is optionally substituted with one, two, three, four, or five groups independently selected from arylC₁-C₆alkoxy and hydroxy;
R^{15′} is hydrogen or C₁-C₆alkyl; or R¹⁵ and R^{15′}, together with the atoms to which they are attached, form a C₃-C₈cycloalkyl ring; and
R^{15″} is hydrogen; amincarbonyl, carboxy, or —(CH₂)ₙC(O)NHCHR¹⁶R^{16′}; wherein
n is 0, 1, or 2;
R¹⁶ is selected from hydrogen, C₁-C₆alkoxyC₁-C₆alkyl, C₁-C₆alkylC₂-C₆alkynyl, C₂-C₆alkynyl, aminoC₁-C₆alkyl, arylC₁-C₆alkyl, carboxy, carboxyC₁-C₆alkyl, heteroaryl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl and the heteroaryl are optionally substituted with one, two, three, four, or five groups independently selected from C₁-C₆alkyl, arylC₁-C₆alkoxy, and hydroxy; and
R^{16′} is hydrogen, C₁-C₆alkyl, aminocarbonyl, carboxy, or —(CH₂)ₘC(O)NHCHR¹⁷R^{17′}; wherein
m is 0, 1, or 2;
R¹⁷ is C₂-C₆alkynyl; and
R^{17′} is aminocarbonyl or carboxy; and
R^{a} is hydrogen or C₁-C₆alkyl; or R¹ and R^{a}, together with the atoms to which they are attached, form an azetidine, morpholine, piperidine, piperazine, or pyrrolidine ring, wherein each ring is optionally substituted with an amino or a hydroxy group.

Those of ordinary skill in the art are aware that an amino acid includes a compound represented by the general structure:

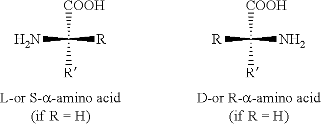

where R and R′ are as discussed herein. Unless otherwise indicated, the term “amino acid” as employed herein, alone or as part of another group, includes, without limitation, an amino group and a carboxyl group linked to the same carbon, referred to as “α” carbon, where R and/or R′ can be a natural or an un-natural side chain, including hydrogen. The absolute “S” configuration at the “α” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Methods

As demonstrated herein, the compounds of the present disclosure are capable of binding to PD-1, disrupting the interaction between PD-1 and PD-L1, competing with the binding of PD-1 with anti-PD-1 monoclonal antibodies that are known to block the interaction with PD-L1, and enhancing CMV-specific T cell IFNγ secretion. As a result, the compounds of the present disclosure are useful for modifying an immune response, treating diseases such as cancer, stimulating a protective autoimmune response, or to stimulate antigen-specific immune responses (e.g., by co-administration of PD-L1 blocking compounds with an antigen of interest).

In another aspect, the present disclosure provides a method of enhancing, stimulating, and/or increasing an immune response in a subject in need thereof, wherein the method comprises administering to the subject a therapeutically effective amount of a compound of Formula (I), as disclosed herein, or a pharmaceutically acceptable salt thereof. In a first embodiment this method further comprises administering an additional agent prior to, after, or simultaneously with the compound of Formula (I), compound of Formula (I)), or a pharmaceutically acceptable salt thereof. In a second embodiment the additional agent is selected from an antimicrobial agent, an antiviral agent, a cytotoxic agent, a TLR7 agonist, a TLR8 agonist, an HDAC inhibitor, a STING agonist, and an immune response modifier.

The present disclosure also provides a method of inhibiting growth, proliferation, or metastasis of cancer cells in a subject in need thereof, said method comprising administering to the subject a therapeutically effective amount a compound of Formula (I), or a pharmaceutically acceptable salt thereof. In a first embodiment of this aspect the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In another aspect the present disclosure provides a method of treating an infectious disease in a subject in need thereof, the method comprising administering to the subject a therapeutically effective amount of a compound of Formula (I), or a pharmaceutically acceptable salt thereof. In a first embodiment of the fourth aspect the infectious disease is caused by a virus. In a second embodiment the virus is selected from HIV, Hepatitis A, Hepatitis B, Hepatitis C, herpes viruses, and influenza.

In another aspect the present disclosure provides a method of treating septic shock in a subject in need thereof, the method comprising administering to the subject a therapeutically effective amount of a compound of Formula (I), or a pharmaceutically acceptable salt thereof.

In another aspect, the present disclosure provides a method of blocking the interaction of PD-1 with PD-L1 in a subject, wherein the method comprises administering to the subject a therapeutically effective amount of a compound of Formula (I), as disclosed herein, or a pharmaceutically acceptable salt thereof.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocyclic peptides described herein, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

### Pharmaceutical Compositions

In another aspect, the present disclosure provides a composition, e.g., a pharmaceutical composition, containing one or a combination of the compounds described within the present disclosure, formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some embodiments, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some embodiments, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, anti-oxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least none emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some embodiments, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one embodiment, a therapeutic composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p 120 (Schreier et al., J. Biol. Chem., 269:9090 (1994)); see also Keinanen, K. et al., FEBS Lett., 346:123 (1994); Killion, J. J. et al., Immunomethods 4:273 (1994).

In certain embodiments, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some embodiments, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### EXAMPLES

The following examples are included to demonstrate various aspects of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and examples are defined as follows: Ph=phenyl; Bn=benzyl; i-Bu=iso-butyl; i-Pr=iso-propyl; Me=methyl; Et=ethyl; Pr=n-propyl; Bu=n-butyl; t-Bu=tert-butyl; Trt=trityl; TMS=trimethylsilyl; TIS=triisopropylsilane; Et₂O=diethyl ether; HOAc or AcOH=acetic acid; MeCN or AcCN=acetonitrile; DMF=N,N-dimethylformamide; EtOAc=ethyl acetate; THE=tetrahydrofuran; TFA=trifluoroacetic acid; TFE=α,α,α-trifluoroethanol; Et₂NH=diethylamine; NMN=N-methylmorpholine; NMP=N-methylpyrrolidone; DCM=dichloromethane; TEA=trimethylamine; min.=minute(s); h or hr=hour(s); L=liter; mL or ml=milliliter; μL=microliter; g=gram(s); mg=milligram(s); mol=mole(s); mmol=millimole(s); meq=milliequivalent; rt or RT=room temperature; sat or sat′d=saturated; aq.=aqueous; mp=melting point; BOP reagent=benzotriazol-1-yloxy-tris-dimethylamino-phosphonium hexafluorophosphate (Castro's reagent); PyBOP reagent=benzotriazol-1-yloxy-tripyrrolidino phosphonium hexafluorophosphate; HBTU=2-(1H-Benzotriazol-1-yl)-1,1,3,3-tetramethyluronim hexafluorophosphate; HATU=O-(7-Azabenzotriazol-1-yl)-1,1,3,3-tetramethyluronim hexafluorophosphate; HCTU=2-(6-Chloro-1-H-benzotriazol-1-yl)-1,1,3,3-tetramethyluronium hexafluorophosphate; T3P=2,4,6-tripropyl-1,3,5,2,4,6-trioxatriphosphorinane-2,4,6-trioxide; DMAP=4-(dimethylamino)pyridine; DIEA=diisopropylethylamine; Fmoc or FMOC=fluorenylmethyloxycarbonyl; Boc or BOC=tert-butyloxycarbonyl; HOBT or HOBT·H₂O=1-hydroxybenzotriazole hydrate; Cl-HOBt=6-Chloro-benzotriazole; HOAT=1-hydroxy-7-azabenzotriazole; HPLC=high performance liquid chromatography; LC/MS=high performance liquid chromatography/mass spectrometry; MS or Mass Spec=mass spectrometry; NMR=nuclear magnetic resonance; Sc or SC or SQ=sub-cutaneous; and IP or ip=intra-peritoneal.

### Example 1. General Synthetic Procedures and Analytical Methods

### Compound Synthesis

The macrocyclic compounds of the present disclosure can be produced by methods known in the art, such as they can be synthesized chemically, recombinantly in a cell free system, recombinantly within a cell or can be isolated from a biological source. Chemical synthesis of a macrocyclic compounds of the present disclosure can be carried out using a variety of art recognized methods, including stepwise solid phase synthesis, semi-synthesis through the conformationally-assisted re-ligation of chemical fragments, enzymatic ligation of cloned or synthetic chemical segments, and chemical ligation. A preferred method to synthesize the macrocyclic compounds and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the a-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The compounds can be synthesized in a stepwise manner on an insoluble polymer support (also referred to as “resin”) starting from the C-terminus of the peptide. A synthesis begins by appending the C-terminal amino acid of an amino acid or peptide to the resin through formation of an amide or ester linkage. This allows the eventual release of the resulting peptide as a C-terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire compound is assembled. The compound is then released from the resin with concomitant deprotection of the side chain functionalities, usually in the presence of appropriate scavengers to limit side reactions. The resulting compound is finally purified by reverse phase HPLC.

The synthesis of the peptidyl-resins required as precursors to the final compounds utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-Cl-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the compound analogs described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown in Table 1.

TABLE 1
Examples of Orthogonally Protected Amino Acids used in Solid Phase Synthesis

| | | | |
|---|---|---|---|
| 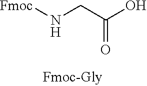 | 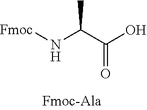 | 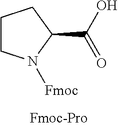 | 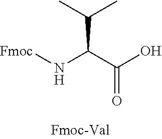 |
| 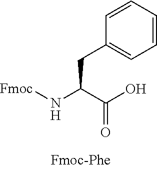 | 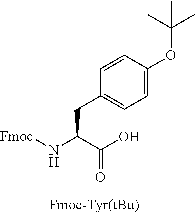 | 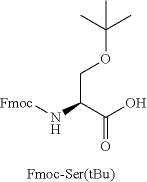 | 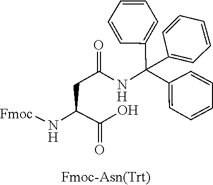 |
| 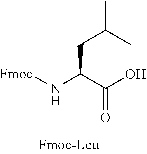 | 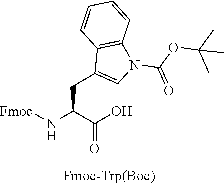 | 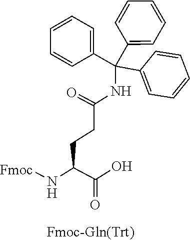 | 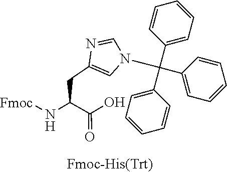 |
| 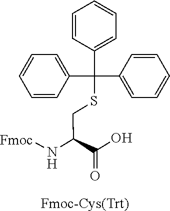 | 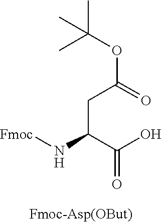 | 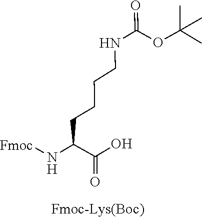 | 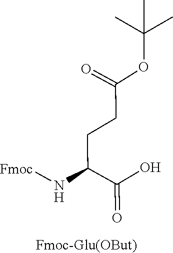 |

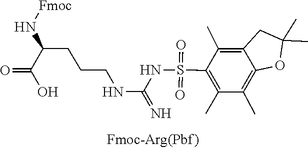

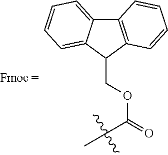 [SMILES: *C(=O)OCC1c2ccccc2-c2ccccc21]

The peptidyl-resin precursors for their respective peptides may be cleaved and deprotected using any standard procedure (see, for Compound, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude peptide is either redissolved directly into DMF or DMSO or CH₃CN/H₂O for purification by preparative HPLC or used directly in the next step.

Peptides with the desired purity can be obtained by purification using preparative HPLC, for Compound, on a Waters Model 4000 or a Shimadzu Model LC-8A liquid chromatography. The solution of crude peptide is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified peptides can be confirmed by electro-spray MS analysis.

A list of unnatural amino acids referred to herein is provided in Table 2:

TABLE 2
Unnatural Amino Acids

| 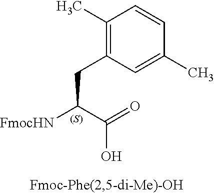 | 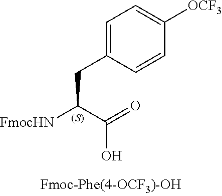 | 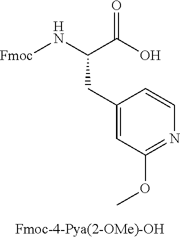 |
|---|---|---|
| 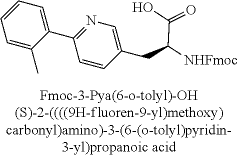 | 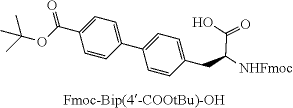 | 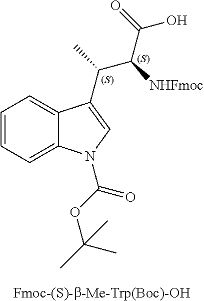 |
| 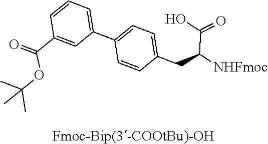 | 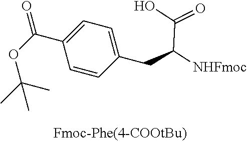 | 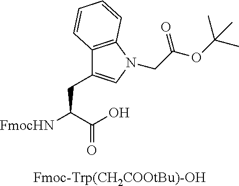 |
| 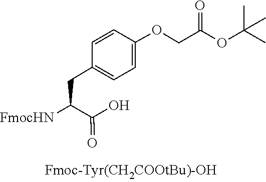 | 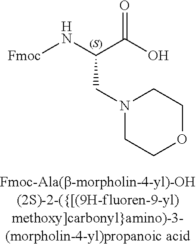 | 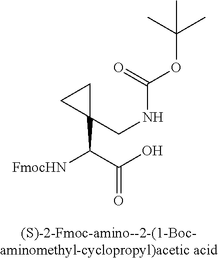 |
| 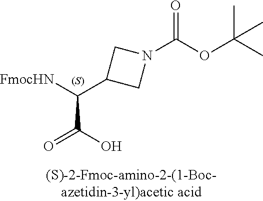 | 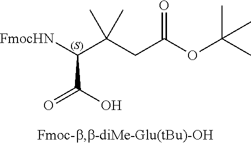 | 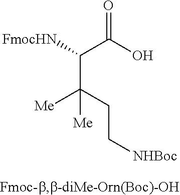 |
| 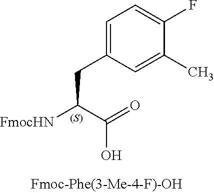 | 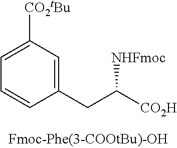 | 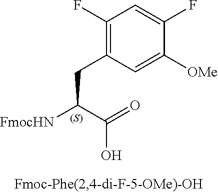 |
| 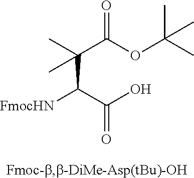 | 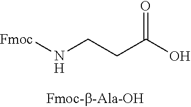 | 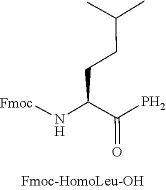 |
| 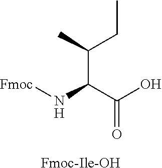 | 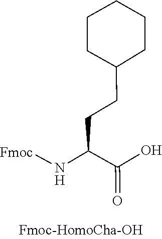 | 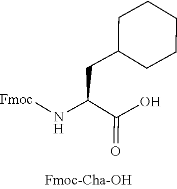 |
| 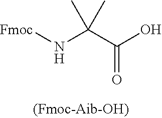 | 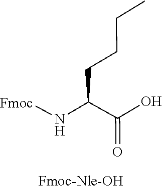 | 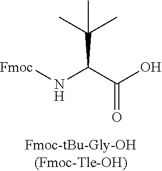 |
| 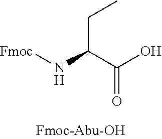 | 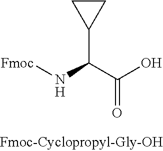 | 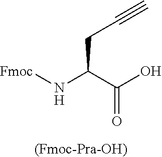 |
| 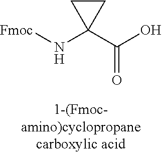 | 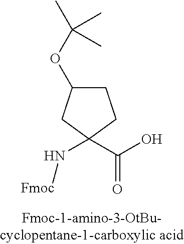 | 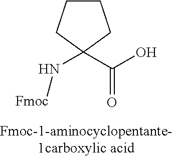 |
| 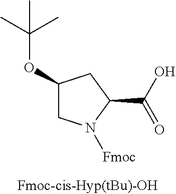 | 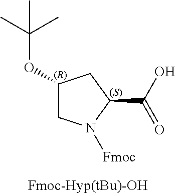 | 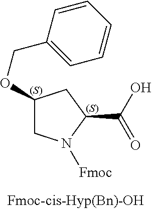 |
| 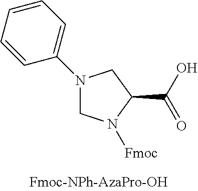 | 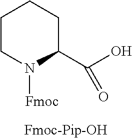 | 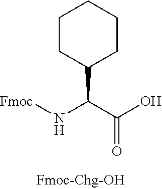 |
| 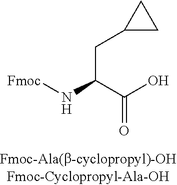 | 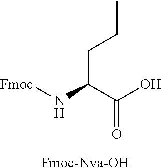 | 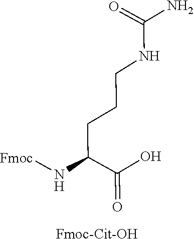 |
| 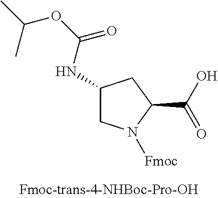 | 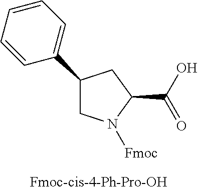 | 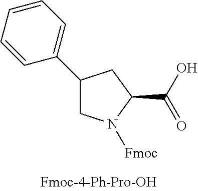 |
| 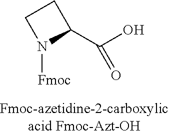 | 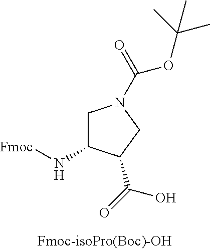 | 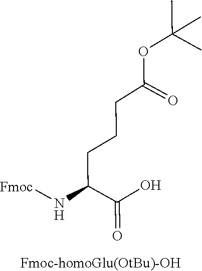 |
| 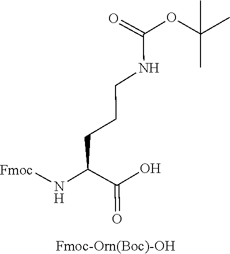 | 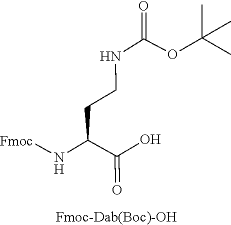 | 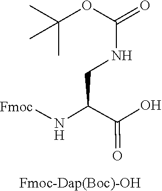 |
| 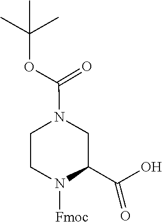 | 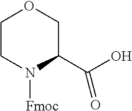 | 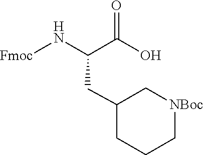 |
| 1-Fmoc-4-(tert-butoxycarbonyl) | 4-N-Fmoc-morpholinecarboxylic | Fmoc-Ala(β-1-Boc-piperidin-3-yl)-OH |
| piperazine-2-carboxylic acid | acid | |
| 1-Fmoc-4-Boc-piperazine-OH | | |
| 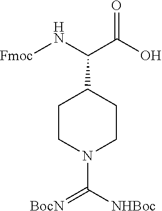 | 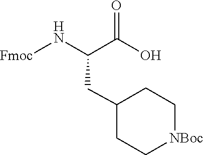 | 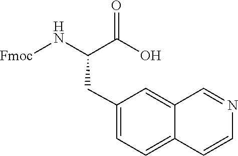 |
| (2S)-2-{1-[(1Z)-{[(tert- | NBoc | Fmoc-Ala(β- |
| butoxy)carbonyl]amino}{{[(tert- | Fmoc-Ala(β-1-Boc-piperidin-4-yl)-OH | isoquinolin-7-yl)-OH |
| butoxy)carbonyl]imino})methyl] | | |
| piperidin-4-yl}-2- | | |
| ({[(9H-fluoren-9-yl)methoxy] | | |
| carbonyl}amino)acetic acid | | |
| 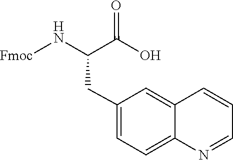 | 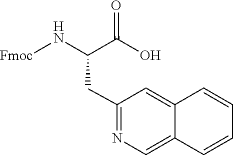 | 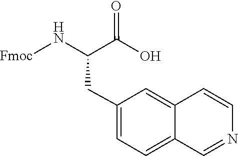 |
| Fmoc-Ala(ß-quinolin-6-yl)-OH | Fmoc-Ala(β-isoquinolin-3-yl)-OH | Fmoc-Ala(β- |
| | | isoquinolin-6-yl)-OH |
| 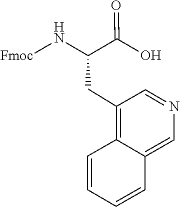 | 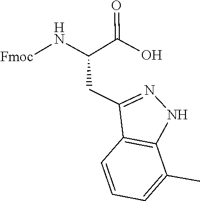 | 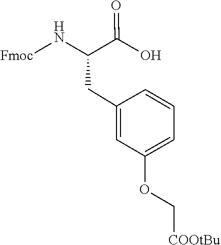 |
| Fmoc-Ala(ß-isoquinolin- | Fmoc-Trp(1,2-N2,7- | Fmoc-Phe(3- |
| 4-yl)-OH | Me)-OH | OCH₂COOtBu)-OH |
| 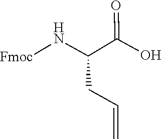 | 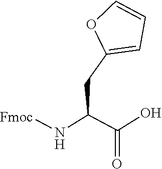 | 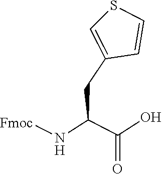 |
| Fmoc-AllyIGly-OH | Fmoc-2-furanyl-Ala-OH | Fmoc-3-thienyl-Ala-OH |
| 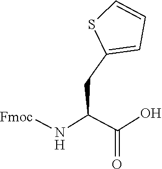 | 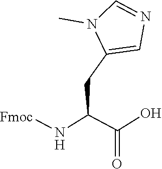 | 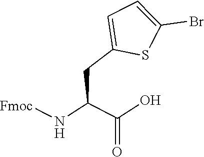 |
| Fmoc-2-thienyl-Ala-OH | Fmoc-His(3-Me)-OH | Fmoc-2-thienyl-5-Br-Ala-OH |
| 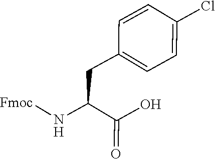 | 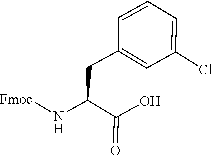 | 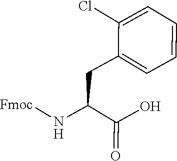 |
| Fmoc-Phe(4-Cl)-OH | Fmoc-Phe(3-Cl)-OH | Fmoc-Phe(2-Cl)-OH |
| 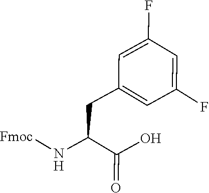 | 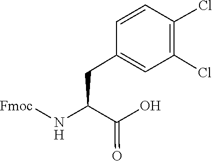 | 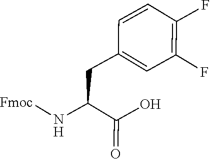 |
| Fmoc-Phe(3,5-di-F)-OH | Fmoc-Phe(3,4-di-Cl)-OH | Fmoc-Phe(3,4-diF)-OH |
| 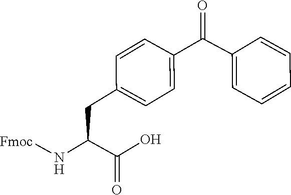 | 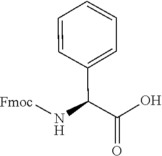 | 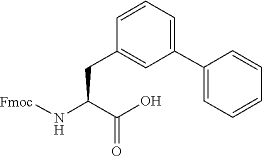 |
| Fmoc-Bpa-OH | Fmoc-PhenylGly-OH | Fmoc-3-Ph-Phe-OH |
| | (Fmoc-Phg-OH) | |
| 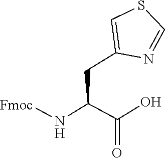 | 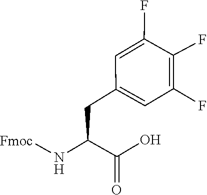 | 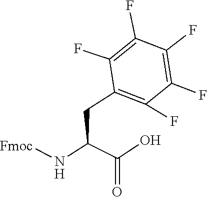 |
| Fmoc-4-Thiazolylalanine | Fmoc-Phe(3,4,5-tri-F)-OH | Fmoc--Phe(2,3,4,5,6- |
| Fmoc-Ala(4-Thiazol-3-yl) | | penta-F)-OH |
| 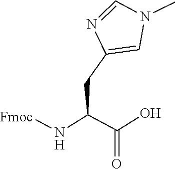 | 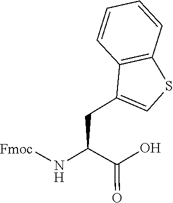 | 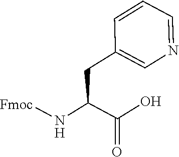 |
| Fmoc-His(1-Me)-OH | Fmoc-Bzt-OH | Fmoc-3-Py-Ala-OH |
| | | (Fmoc-3-Pya-OH) |
| 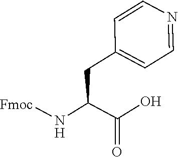 | 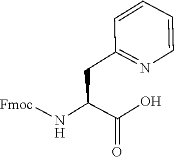 | 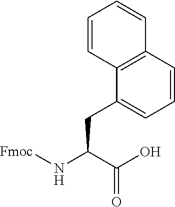 |
| Fmoc-4-Py-Ala-OH | Fmoc-2-Py-Ala-OH | Fmoc-1-Nal-OH |
| (Fmoc-4-Pya-OH) | (Fmoc-2-Pya-OH) | |
| 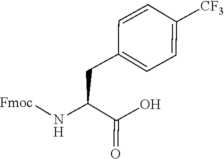 | 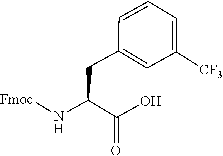 | 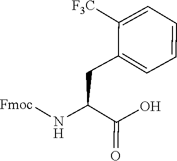 |
| Fmoc-Phe(4-CF₃)-OH | Fmoc-Phe(3-CF₃)-OH | Fmoc-Phe(2-CF₃)-OH |
| 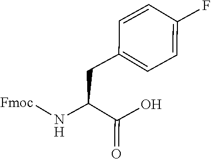 | 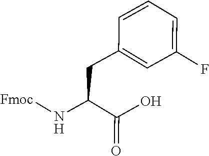 | 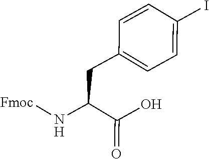 |
| Fmoc-Phe(4-F)-OH | Fmoc-Phe(3-F)-OH | Fmoc-Phe(4-I)-OH |
| 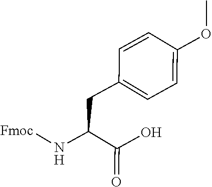 | 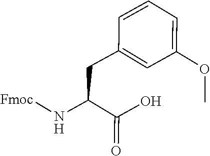 | 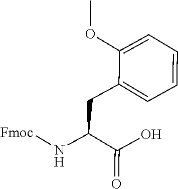 |
| Fmoc-Tyr(Me)-OH | Fmoc-Phe(-3-OMe)-OH | Fmoc-Phe(2-OMe)-OH |
| (Fmoc-Phe(4-OMe)-OH) | | |
| 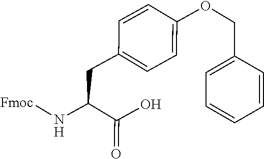 | 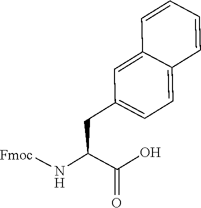 | 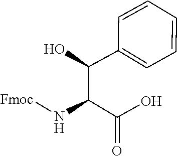 |
| Fmoc-Tyr(Bn)-OH | Fmoc-2-NaI-OH | Fmoc-ß-hydroxy-Phe-OH |
| 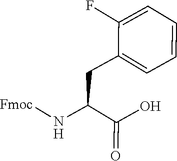 | 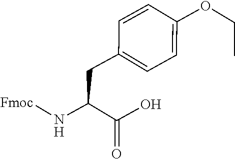 | 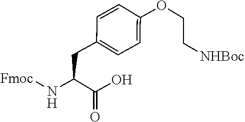 |
| Fmoc-Phe(2-F)-OH | Fmoc-Tyr(Et)-OH | Fmoc-Tyr(CH₂CH₂NHBoc)-OH |
| 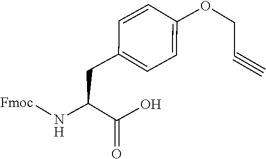 | 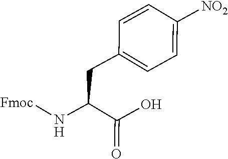 | 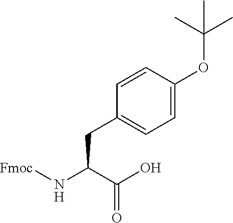 |
| Fmoc-Tyr(propargyl)-OH | Fmoc-Phe(4-NO₂)-OH | Fmoc-Tyr(tBu)-OH |
| 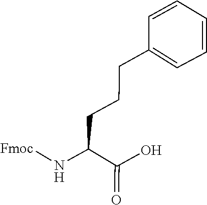 | 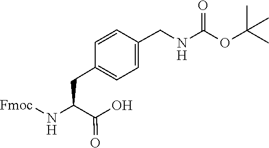 | 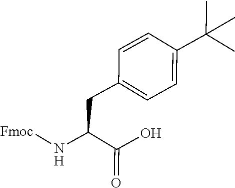 |
| Fmoc-homo-homo-Phe-OH | Fmoc-Phe(4- | Fmoc-Phe(4-tBu)-OH |
| | aminomethyl(Boc))-OH | |
| 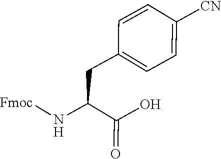 | 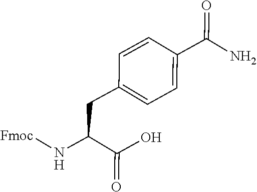 | 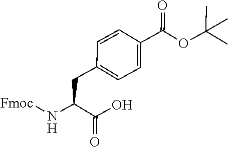 |
| Fmoc-Phe(4-CN)-OH | Fmoc-Phe(4-CONH₂)-OH | Fmoc-Phe(4-COOtBu)--OH |
| 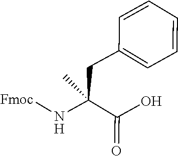 | 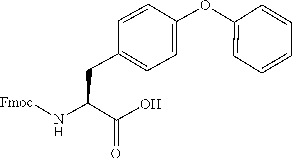 | 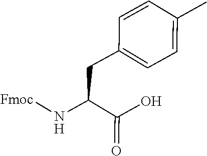 |
| Fmoc-α-Me-Phe-OH | Fmoc-Phe(4-OPh)-OH | Fmoc-Phe(4-Me)-OH |
| | (Fmoc-Tyr(Ph)-OH) | |
| 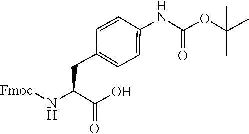 | 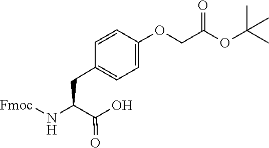 | 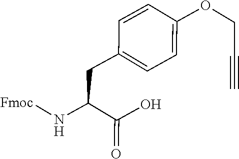 |
| Fmoc-Phe(4-NHBoc)-OH | Fmoc-Tyr(CH₂COOtBu)-OH | Fmoc-Tyr(propargyl)-OH |
| 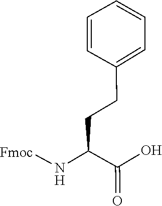 | 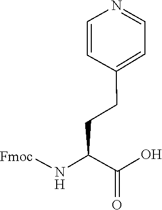 | 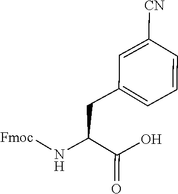 |
| Fmoc-HomoPhe-OH | Fmoc-Homo-4-Py-Ala-OH | Fmoc-Phe(3-CN)-OH |
| 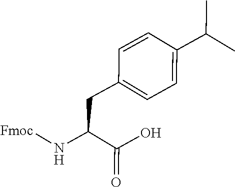 | 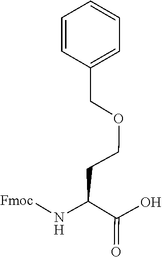 | 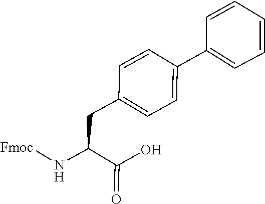 |
| Fmoc-4-Phe(iPr)-OH | Fmoc-Homo-Ser(Bn)-OH | Fmoc-Bip-OH |
| 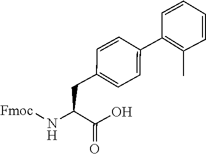 | 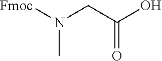 | 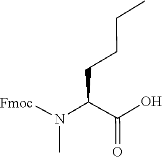 |
| Fmoc-Bip(2'-Me)-OH | Fmoc-N-Me-Gly-OH | Fmoc-N-Me-Nle-OH |
| | (Fmoc-mGly-OH) | (Fmoc-mNle-OH) |
| 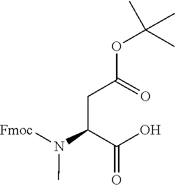 | 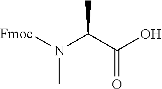 | 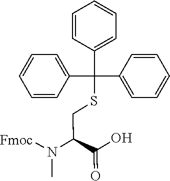 |
| Fmoc-N-Me-Asp(OtBu)-OH | Fmoc-N-Me-Ala-OH | Fmoc-N-Me-Cys(Trt)-OH |
| (Fmoc-mAsp(OtBu)-OH) | (Fmoc-mAla-OH) | (Fmoc-mCys(Trt)-OH) |
| 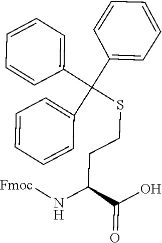 | 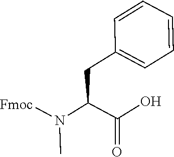 | 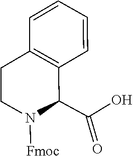 |
| Fmoc-HomoCys(Trt)-OH | Fmoc-N-Me-Phe-OH | Fmoc-Iso-Tic-OH |
| | (Fmoc-mPhe-OH) | |
| 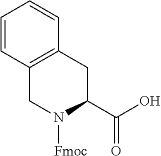 | 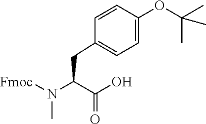 | 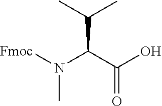 |
| Fmoc-Tic-OH | Fmoc-N-Me-Tyr(OtBu)-OH | Fmoc-N-Me-Val-OH |
| | (Fmoc-mTyr(OtBu)-OH) | (Fmoc-mVal-OH) |
| 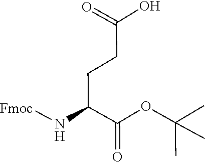 | 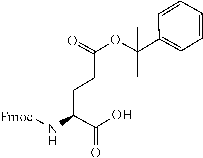 | 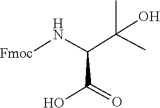 |
| Fmoc-Glu-OtBu | Fmoc-Glu(2- | Fmoc-S-hydroxyvaline |
| | phenylisopropyloxy)-OH | Fmoc-V(β-OH)-OH |
| 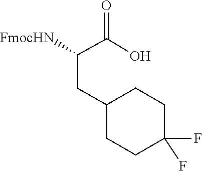 | 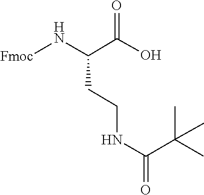 | 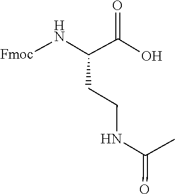 |
| Fmoc-Ala(β-Cha(4,4-di-F))-OH | Fmoc-Dab(COtBu)-OH | Fmoc-Dab(Ac)-OH |
| 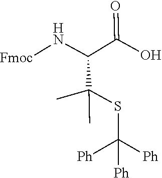 | 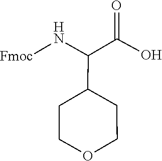 | 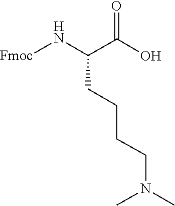 |
| Fmoc-β,β-dimethyl-Cys(Trt)-OH | 2-NHFmoc-2-(oxan-4-yl)acetic | Fmo-Lys(N,N-di-Me)-OH |
| Fmoc-Pen(Trt)-OH | acid | |
| 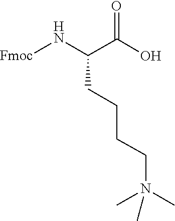 | 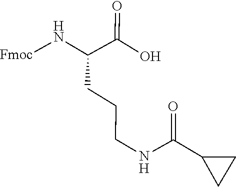 | 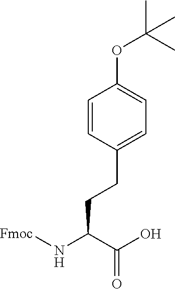 |
| FMOC-Lys(trimethyl)-OH | Fmoc-Orn(CO-cyclopropyl)-OH | Fmoc-Homo Tyr(OtBu)-OH |
| 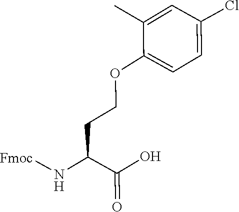 | 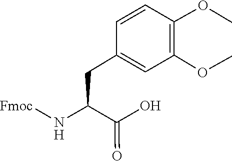 | 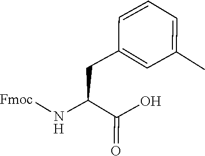 |
| Fmoc-4-Cl-2-Me- | Fmoc-Phe(3,4-di-OMe)-OH | Fmoc-Phe(3-Me)-OH |
| phenoxyhomoSer-OH | | |
| 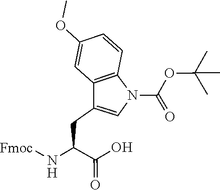 | 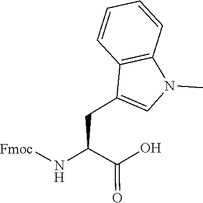 | 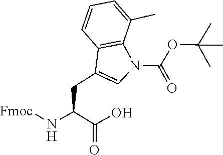 |
| Fmoc-5-MeO-Trp(Boc)-OH | Fmoc-Nme-Trp-OH | Fmoc-7-Me-Trp(Boc)-OH |
| 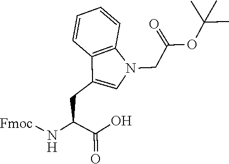 | 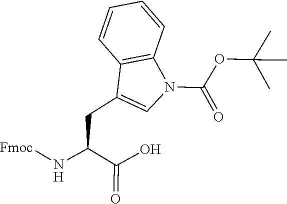 | 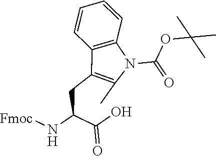 |
| Fmoc-N-AcOH-Trp-OH | Fmoc-Trp(Boc)-OH | Fmoc-2-Me-Trp(Boc)-OH |
| 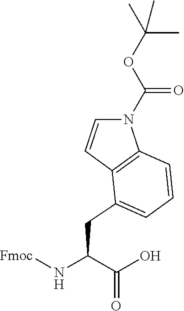 | 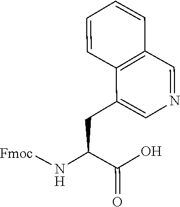 | 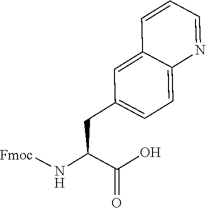 |
| Fmoc-Iso-Trp(Boc)-OH | Fmoc-Ala-(β-isoquinolin-4-yl)-OH | Fmoc-Ala-(β-quinolin-6-yl)-OH |
| 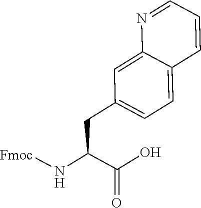 | 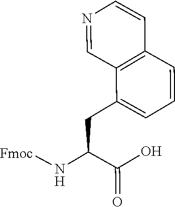 | 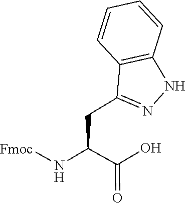 |
| Fmoc-Ala-(β-quinolin-7-yl)-OH | Fmoc-Ala-(β-isoquinolin-8-yl)-OH | Fmoc-Ala(β-indazol-3-yl)-OH |
| 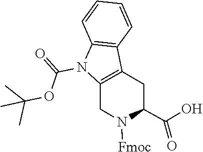 | 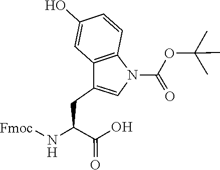 | 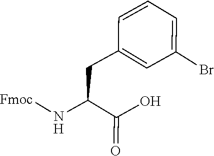 |
| Fmoc-Trp(Boc) Tic-OH | Fmoc-5-OH-Trp(Boc)-OH | Fmoc-Phe(3-Br)-OH |
| 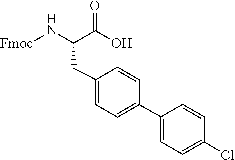 | 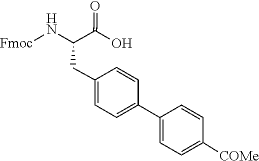 | 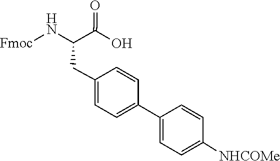 |
| Fmoc-Bip(4'-Cl)-OH | Fmoc-Bip(4'-COMe)-OH | Fmoc-Bip(4'-NHCOMe)-OH |
| 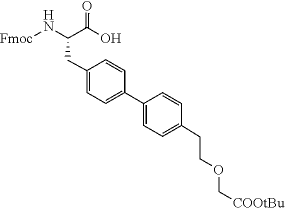 | 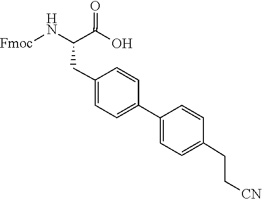 | 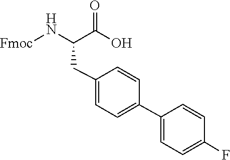 |
| Fmoc-Bip(4'- | Fmoc-Bip(4'-CH₂CH₂CN)-OH | Fmoc-Bip(4'-F)-OH |
| CH₂CH₂OCH₂COOtBu)-OH | | |
| 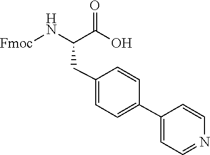 | 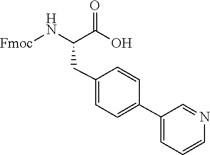 | 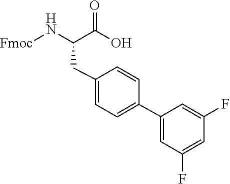 |
| Fmoc-Bip(4'-Pyr)-OH | Fmoc-Bip(3'-Pyr)-OH | Fmoc-Bip(3',5'-F2)-OH |
| 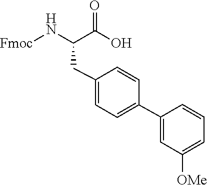 | 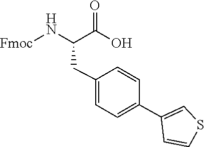 | 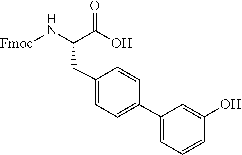 |
| Fmoc-Bip(3'-OMe)-OH OMe | Fmoc-Bip(3'-Thio)-OH | Fmoc-Bip(3'-OH)-OH |
| 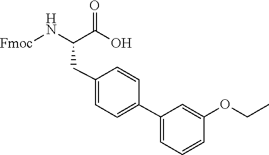 | 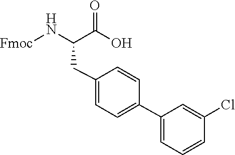 | 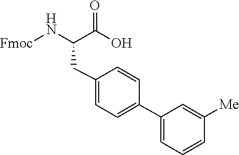 |
| Fmoc-Bip(3'-OEt)-OH | Fmoc-Bip(3'-Cl)-OH | Fmoc-Bip(3'-Me)-OH |
| 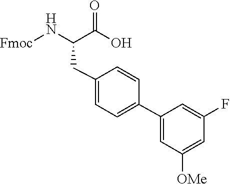 | 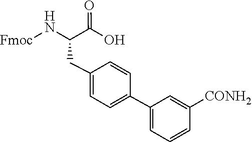 | 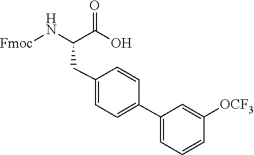 |
| Fmoc-Bip(3'-F,5'-OMe)-OH | Fmoc-Bip(3'-CONH₂)-OH | Fmoc-Bip(3'-OCF₃)-OH |
| 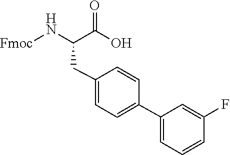 | 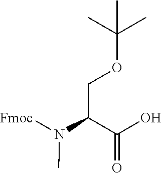 | 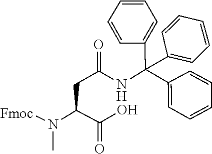 |
| Fmoc-Bip(3'-F)-OH | Fmoc-N-Me-Ser(tBu)-OH | Fmoc-N-Me-Asn(Trt)-OH |
| | (Fmoc-mSer(tBu)-OH) | (Fmoc-mAsn(Trt)-OH) |
| 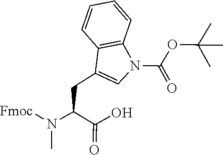 | 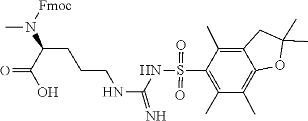 | |
| Fmoc-N-MeTrp(Boc)-OH | Fmoc-N-Me-Arg(Pbf)-OH | |
| (Fmoc-mTrp(Boc)-OH) | (Fmoc-mArf(Pbf)-OH) | |
| 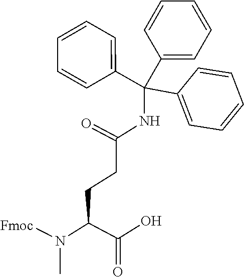 | 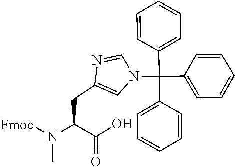 | 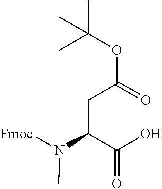 |
| Fmoc-N-Me-Gln(Trt) | Fmoc-N-Me-His(Trt) | Fmoc-N-Me-Asp(OtBu)-OH |
| | | (Fmoc-mAsp(OtBu)-OH) |
| 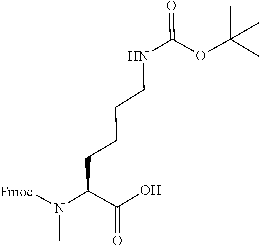 | 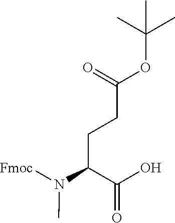 | 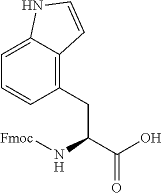 |
| Fmoc-N-Me-Lys(Boc)-OH | Fmoc-N-Me-Glu(OtBu)-OH | 2-(Fmoc-amino)-3-(1H-indol-4- |
| (Fmoc-mLys(Boc)-OH) | (Fmoc-nGlu(OtBu)-OH) | yl)propanoic acid |
| 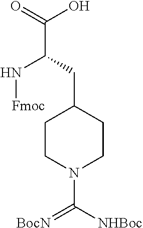 | 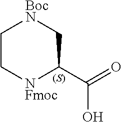 | 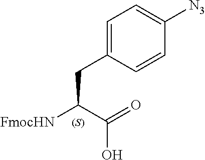 |
| Fmoc-Ala(β-1- | (S)-1-Fmoc-4-Boc- | Fmoc-Phe(4-N₃)-OH |
| carbamimidoylpiperidin-4- | piperazine-2-carboxylic acid | |
| yl)-OH | | |
| 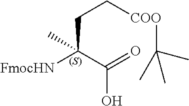 | 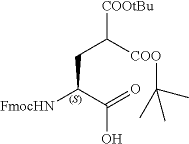 | 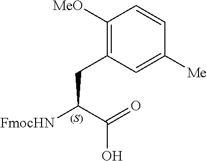 |
| Fmoc-α-Me-Glu(tBu) | Fmoc-β-COOtBu-Glu(tBu) | Fmoc-Phe(2-OMe-5-Me)-OH |
| 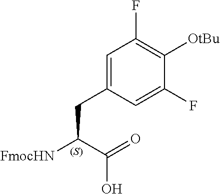 | | |
| Fmoc-Tyr(tBu, 2,6-di-F)-OH | | |

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “in z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitril e: water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LC/MS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LC/MS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LC/MS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LC/MS Condition F.

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LC/MS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LC/MS Condition H:

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LC/MS Condition I:

Column: X Bridge C18, 4.6×50 mm, 5-μm particles; Mobile Phase A: 10 mM NH₄OAc; Mobile Phase B: acetonitrile, Temperature: 35° C.; Gradient: 5-95% B over 4 minutes; Flow: 4.0 mL/min; Detection: UV at 220 nm.

### Analytical LC/MS Condition J.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LC/MS Condition K.

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 100% B; Flow: 0.80 mL/min; Detection: UV at 220 nm.

### Analytical LC/MS Condition L:

Column: Waters Acquity UPLC BEH C18, 2.1×50 mm, 1.7-μm particles; Buffer:10 mM Ammonium Acetate. Mobile Phase A: buffer” CH₃CN (95/5); Mobile Phase B: Mobile Phase B:Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LC/MS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis. Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)-used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NN (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel was remain attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), and then the vessel was closed. The automatic program was resumed and NN (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below.

### Resin-Swelling Procedure:

To a 25-mL polypropylene solid-phase reaction vessel was added Sieber resin (70 mg, 0.05 mmol). The resin was washed (swelled) as follows: to the reaction vessel was added DMF (2.0 mL), upon which the mixture was periodically agitated for 10 minutes before the solvent was drained through the frit.

### Single-Coupling Procedure:

To the reaction vessel containing the resin from the previous step was added DMF (2.5 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). The mixture was periodically agitated for 5.0 minutes and then the solution was drained through the frit. The resin was washed successively six times as follows: for each wash, DMF (2.5 mL) was added to the vessel and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NN (0.8 M in DMF, 1.25 mL, 20 equiv). The mixture was periodically agitated for 30-120 minutes, then the reaction solution was drained through the frit. The resin was washed successively six times as follows: for each wash, DMF (2.5 mL) was added and the resulting mixture was periodically agitated for 30 seconds before the solution was drained through the frit. The resulting resin was used directly in the next step.

### Single-Coupling Pre-Activation Procedure:

To the reaction vessel containing the resin from the previous step was added DMF (3.75 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). To the resin was added piperidine:DMF (20:80 v/v, 3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the premixed amino acid and HATU (0.1 M in DMF, 1.25 mL, 1:1 ratio 2.5 equiv), then NMM (0.8 M in DMF, 1.25 mL, 20 equiv). The mixture was periodically agitated for 2-3 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5 mL) three times, upon which the mixture was agitated for 30 seconds before the solvent was drained through the frit each time. To the reaction vessel was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NN (0.8 M in DMF, 1.25 mL, 20 equiv). The mixture was periodically agitated for 1 hour, then the reaction solution was drained through the frit. The resin was washed twice with DMF (3.75 mL) and the resulting mixture was periodically agitated for 30 seconds before the solution was drained through the frit each time. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 eq). The mixture was periodically agitated for 1-2 hours, then the reaction solution was drained through the frit. The resin was successively washed six times as follows: for each wash, DMF (3.75 mL) was added and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.75 mL) three times, upon which the mixture was agitated for 30 seconds before the solvent was drained through the frit each time. To the reaction vessel containing the resin from the previous step was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NN (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis:

Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel was remain attached to the instrument, followed by the manual addition of HATU (2-4 equiv, same equiv as the unnatural amino acid), then the vessel was closed. The automatic program was resumed and NN (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml bio-rad poly-prep chromatography column was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the globle deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on speedvac or genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1,v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on speedvac or genevac EZ-2. The resulting residue was charged with CH₃CN:H₂O (2:3, v/v, 30 mL), and concentrated to dryness on speedvac or genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

N-Methylation on-resin Method A.

To the resin (50 μmol) in a Bio-Rad tube was added CH₂Cl₂ (2 mL) and shaken for 5 min at rt. 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at rt for 2 h. The solvent was drained and the resin was rinsed with CH₂Cl₂ (5 mL×3), DMF (5 mL×3) and then THF (5 mL×3). The resin was added THE (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at rt for 1-16 h. The solvent was drained, and the resin was washed with THE (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×). Air dried and used directly in the next step.

### N-Methylation On-Resin Method B

(Turner, R. A. et al, Org. Lett., 15(19):5012-5015 (2013)). All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0. 119 ml, 1.00 mmol), 1,8-diazabicyclo[5.4.0]undec-7-ene (0.181 ml, 1.20 mmol). The mixture was placed on a shaker for 60 min. The solution was drained through the frit. The resin was washed successively three times with DMF (4.0 mL) and three times with DCM (4.0 mL). The resin was washed three times with dry THE (2.0 mL) to remove any residual water. In an oven dried 4.0 mL vial was added THE (1.0 mL) and triphenylphosphine (131 mg, 0.500 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed with three times with dry THE (2.0 mL) to remove any residual water. In an oven dried 4.0 mL vial was added THE (1.0 mL), triphenylphosphine (131 mg, 0.50 mmol) on dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THE (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THE was added to nosylated resin (0.186 g, 0.100 mmol), and the reaction mixture was stirred for 16 hours at room temperature. The resin was washed three times with THE (5 mL) Tetrahydrofuran, and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMIP (3 mL). A solution of NMIP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMIP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preload Amines on the PL-FMP Resin:

PL-FMP resin (Novabiochem, 1.00 mmol/g substitution) was swollen with DMF (20 mL/mmol) at room temperature. The solvent was drained and 10 ml of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed by DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (1×), and DCM (3×). The sample was then cleaved with 40% TFA/DCM (1 h). The product was collected and analyzed by HPLC and MS. Collected sample was dried and got weight to calculate resin loading.

### Click Reaction On-Resin Procedure Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2×5 mL×5 mins). In a 200-ml bottle was charged with 30 time of the following: vitamin C (0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THE (1.5 ml), followed by DIPEA (0.087 ml, 0.50 mmol) and the azide, tert-butyl (S)-1-azido-40-(tert-butoxycarbonyl)-37,42-dioxo-3,6,9,12,15,18,21,24,27,30,33-undecaoxa-36,41-diazanonapentacontan-59-oate (0.028 g, 0.025 mmol). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Procedure Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swell with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 uL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 uL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-resin Procedure:

In a Bio Rad tube is placed 50 umoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1,1′-bis(di-tert-butylphosphino)ferrocene]dichloropalladium(II) [PdCl₂(dtbpf)](3.26 mg, 5.00 μmol). The tube was shaken at rt overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 ml TFA at rt for 1 h. The rest of the resin was used in the next step of peptide coupling or chloroacetic acid capping of the N-terminus.

### Preparation of (S)-2-((((9H-fluoren-9-yl)methoxy)carbonyl)-3-(1-(2-(tert-butoxy)-2-oxoethyl)-1H-indol-3-yl)propanoic acid

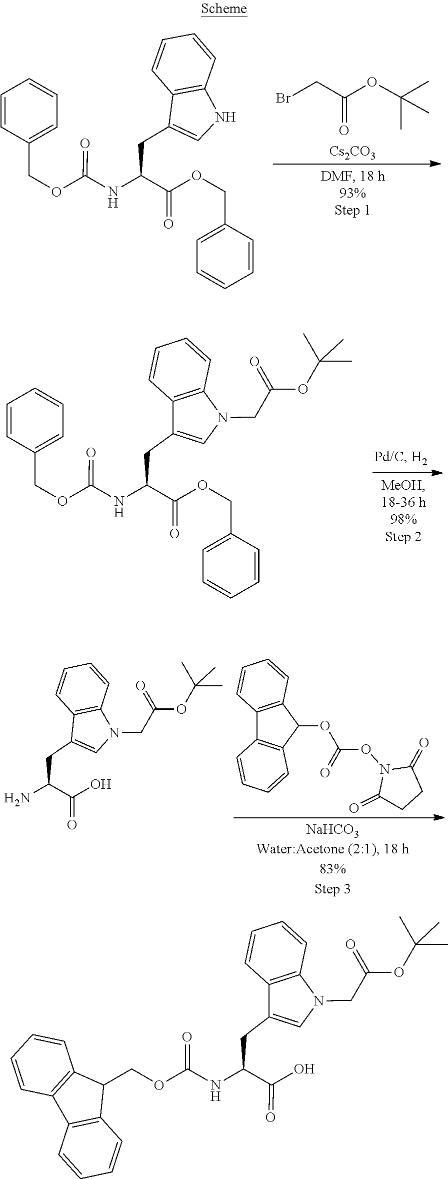

### Step 1:

To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

### Step 2:

H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd—C (1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

### Step 3:

To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic acid

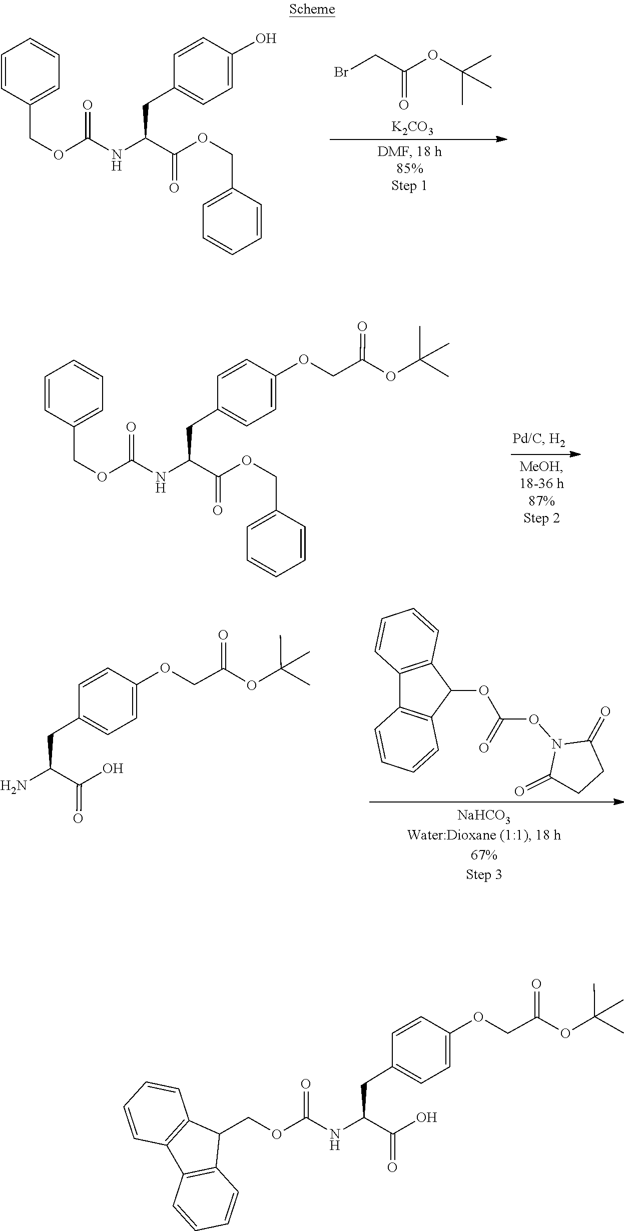

### Step 1:

To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford colorless gum. The crude compound was purified by flash column chromatography using 20% ethyl acetate in petroleum ether as an eluent to afford a white solid (78 g, 85%).

### Step 2:

The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite©) and washed with methanol (1000 mL). The filtrate was concentrated under vacuum to afford a white solid (36 g, 87%).

### Step 3:

To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of (S)-2-((((9H-fluoren-9-yl)methoxy)carbonyl)amino)-3-(4-(tert-butoxycarbonyl)phenyl)propanoic acid

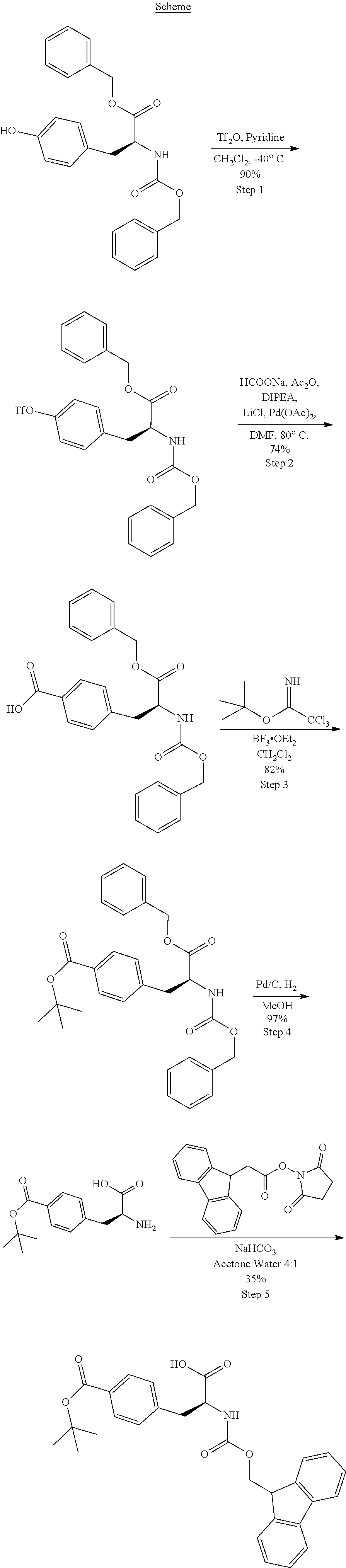

### Step 1.

(S)-Benzyl 2-(((benzyloxy)carbonyl)amino)-3-(4-hydroxyphenyl)propanoate (10 g, 24.66 mmol) was taken in DCM (100 mL) in a 250 mL multi-neck round bottom flask under magnetic stirring with N₂ outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at −40° C. for 2 hours. The reaction mixture was quenched with water at −10° C., and then added citric acid solution (50 mL). The organic layer was extracted in DCM, and the separated organic layer was dried over anhydrous Na₂SO₄, filtered, and then evaporated to give (S)-benzyl 2-(((benzyloxy)carbonyl)amino)-3-(4-(((trifluoromethyl)sulfonyl)oxy)phenyl)propanoate (11.93 g, 22.20 mmol, 90% yield) as a pale yellow solid.

### Step 2.

A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy)carbonyl)amino)-3-(4-(((trifluoromethyl)sulfonyl)oxy)phenyl)propanoate (300 g, 558 mmol), lithium chloride (71 g, 1675 mmol), 1,3-bis(diphenylphosphino)propane (24.17 g, 58.6 mmol) were added followed by the addition of palladium(II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%).

### Step 3.

To a 2000-ml multi-neck round-bottomed flask was charged (S)-4-(3-(benzyloxy)-2-(((benzyloxy)carbonyl)amino)-3-oxopropyl)benzoic acid (130 g, 300 mmol), dichloromethane (260 mL) and cyclohexane (130 mL). To the slurry reaction mixture was added BF₃·OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using 1.5 Kg silicycle column. The product spot was eluted at 15% ethyl acetate/petroleum ether mixture. The collected fractions were concentrated to obtain a colorless liquid (120 g, yield 82%).

### Step 4.

(S)-tert-Butyl 4-(3-(benzyloxy)-2-(((benzyloxy)carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H₂ for 16 h at room temperature. The reaction mass was filtered through celite bed and the bed was washed with methanol. The obtained filtrate was concentrated to obtain a pale yellow solid. The obtained solid was stirred with 5% methanol: diethyl ether mixture for 15 min before being filtered, dried under vacuum to obtain a pale yellow solid. It was made slurry with 5% methanol in diethyl ether and stirred for 15 min, filtered, and dried to give (S)-2-amino-3-(4-(tert-butoxycarbonyl)phenyl)propanoic acid as a white solid (105 g, yield 97%). Analysis condition E: Retention time=0.971 min; ESI-MS(+) m/z [M+H]⁺: 266.2.

### Step 5.

(S)-2-Amino-3-(4-(tert-butoxycarbonyl)phenyl)propanoic acid (122 g, 460 mmol) was dissolved in acetone (1000 mL) and then water (260 mL) and sodium bicarbonate (116 g, 1380 mmol) were added. It was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to get the desired product as a white solid (80 g, yield 35%). ¹H NMR (400 MHz, DMSO-d6) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl)methoxy)carbonyl)amino)-3-iodopropanoate

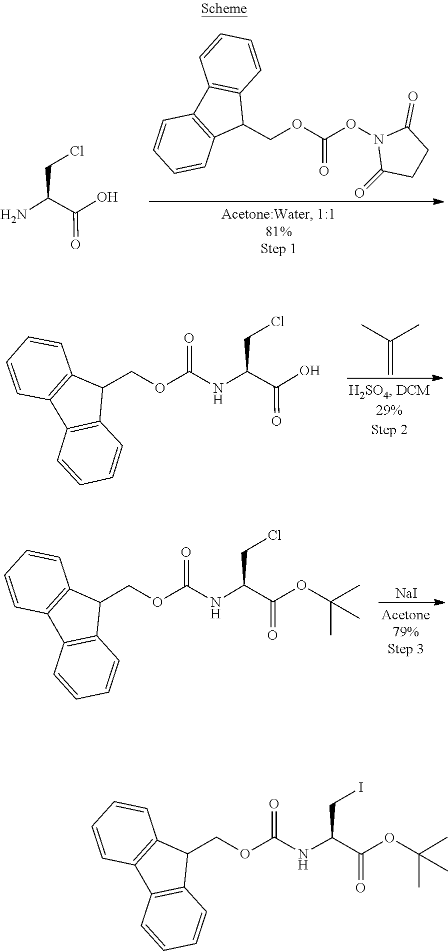

### Step 1.

To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl)methoxy)carbonyl)amino)-3-chloropropanoic acid. The product (220 g) was taken to the next step as such.

### Step 2.

A solution of (R)-2-((((9H-fluoren-9-yl)methoxy)carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H₂SO₄ (57.7 mL, 1082 mmol) was added and the mixture was stirred at RT overnight. To the reaction mixture was added water (500 mL). The layers were separated and the aqueous layer was extracted DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl)methoxy)carbonyl)amino)-3-chloropropanoate (83 g, 182 mmol, 29% yield).

### Step 3.

To a solution of (R)-tert-butyl 2-((((9H-fluoren-9-yl)methoxy)carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl)methoxy)carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl)methoxy)carbonyl)amino)-3-(2-methyl-1H-indol-3-yl)propanoic acid

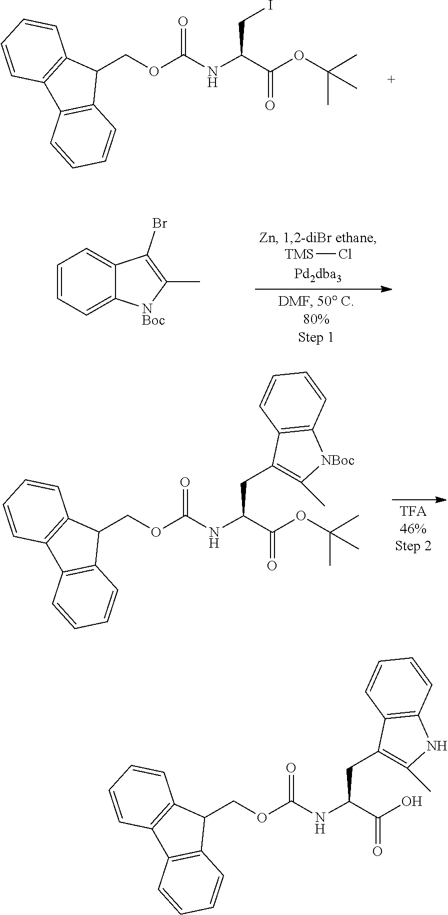 [SMILES: CC(C)(C)OC(=O)[C@H](CI)NC(=O)OCC1c2ccccc2-c2ccccc21 ; Cc1c(Br)c2ccccc2n1C(=O)OC(C)(C)C ; C[Si](C)(C)Cl ; Cc1c(C[C@H](NC(=O)OCC2c3ccccc3-c3ccccc32)C(=O)OC(C)(C)C)c2ccccc2n1C(=O)OC(C)(C)C ; Cc1[nH]c2ccccc2c1C[C@H](NC(=O)OCC1c2ccccc2-c2ccccc21)C(=O)O]

### Step 1.

In a 100-ml three-neck, flame-dried, nitrogen-purged round-bottomed flask, zinc (2.319 g, 35.5 mmol) was added under argon atmosphere and the flask was heated to 150° C. using a hot gun and was purged with argon. To the reaction flask, DMF (50 mL) was added followed by the addition of 1,2-dibromoethane (0.017 mL, 0.20 mmol) and TMS-Cl (0.026 mL, 0.20 mmol) under argon atmosphere and then stirred for 10 min. To the reaction mixture (R)-tert-butyl 2-((((9H-fluoren-9-yl)methoxy)carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone)dipalladium(0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through Celite. The organic phase was washed with sat. NH₄Cl (250 mL), water (2×200 mL), and sat. NaCl (aq) (250 mL), dried over anhydrous Na₂SO₄(s), concentrated, and dried under vacuum to afford the crude compound (19 g). It was purified through ISCO flash chromatography using 330 g redisep column and the product was eluted with 7 to 9% of ethyl acetate in petroleum ether. The above reaction and purification were repeated. The pure fractions were concentrated to give tert-butyl (S)-3-(2-((((9H-fluoren-9-yl)methoxy)carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M−Boc−tBu+H]⁺: 441.2.

### Step 2.

In a 25-ml multi neck, round-bottomed flask, DCM (65 mL) was added followed by (S)-tert-butyl 3-(2-((((9H-fluoren-9-yl)methoxy)carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in cold room to give a brown colored solid (crude weight: 6.5 g). It was purified via reverse phase flash chromatography, and the pure fractions were concentrated to obtain the desired final product as an off-white powder (2.3 g, 46%). ¹H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl)methoxy)carbonyl)amino)-3-(7-methyl-1H-indol-3-yl)propanoic acid

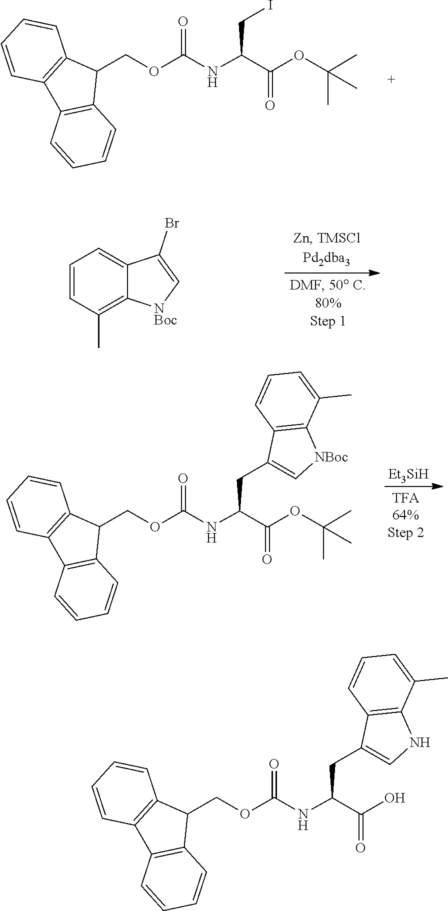

### Step 1.

In a 50-mi round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min (R)-tert-butyl 2-((((9H-fluoren-9-yl)methoxy)carbonyl)amino)-3-iodopropanoate (2.0 g, 4.05 mmol) was added and the reaction was stirred for 30 min. In a 50-ml round-bottomed flask equipped charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂(dba)₃ (0.093 g, 0.101 mmol). After the addition the reaction mixture was heated to 50° C. overnight. Another equivalents of Sphos and Pd₂(dba)₃ was added and heating continued for another 16 h. The reaction mixture was diluted with EtOAc (100 mL) and filtered through Celite. The organic phase was washed with sat. aq. NH₄Cl (100 mL), water (50 mL), and sat NaCl (100 mL), dried over anhydrous Na₂SO₄(s), concentrated, and dried under vacuum. After purification by flash chromatography the desired tert-butyl (S)-3-(2-((((9H-fluoren-9-yl)methoxy)carbonyl)amino)-3-(tert-butoxy)-3-oxopropyl)-2-methyl-1H-indole-1H-carboxylate was obtained in 58% yield.

### Step2.

Final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(7-methyl-1H-indol-3-yl)propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d6) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H), 0.02-−0.06 (m, 1H).

### Preparation of (S)-2-((((9H-fluoren-9-yl)methoxy)carbonyl)amino)-3-(quinolin-6-yl)propanoic acid

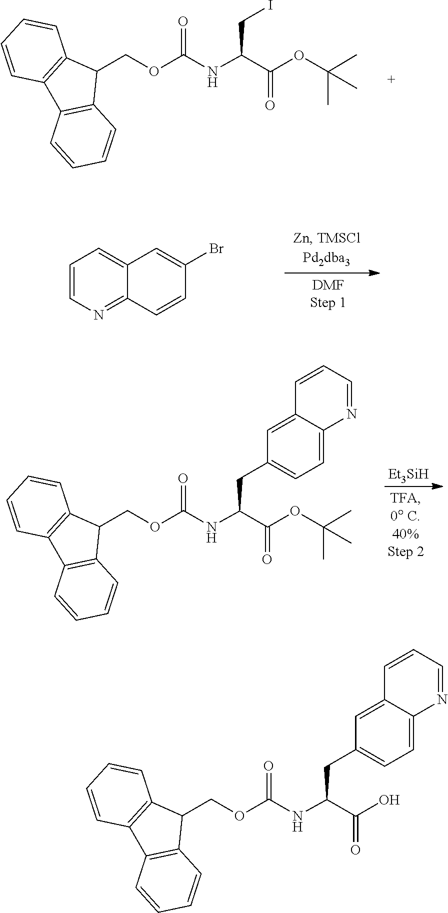

### Step 1.

In a 25-ml round bottom flask, dry zinc (2.32 g, 35.5 mmol) was charged and argon was flashed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl)methoxy)carbonyl)amino)-3-iodopropanoate (5.0 g, 10.14 mmol) was added and the reaction was stirred for 30 min.

In a 250-ml round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl)methoxy)carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at rt for 5 h and then heated to 50° C. for 16 h. It was cooled to rt and filtered over celite and rinsed with ethyl acetate. The solution was concentrated on rotovap. Purification by flash chromatography gave the desired compound as a thick brown liquid in quantitative yields. Analysis condition E: Retention time=3.47 min; ESI-MS(+) m/z [M+H]⁺: 495.2.

### Step2.

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(quinolin-6-yl)propanoic acid as a beige solid in 40% yield after solid-liquid extraction with diethyl ether and water. ¹H NMR (300 MHz, DMSO-d₆) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl)methoxy)carbonyl)amino)-3-(isoquinolin-6-yl)propanoic acid

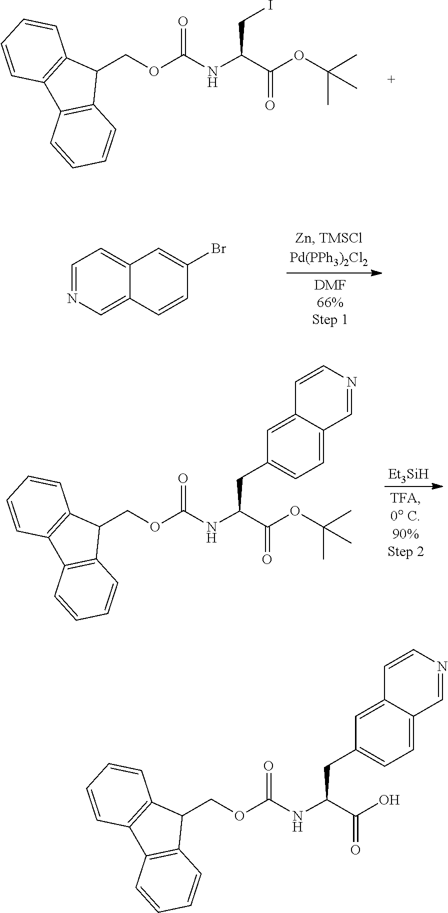

### Step 1.

In a 50-ml three neck flame-dried round bottom flask zinc (1.392 g, 21.28 mmol) was added under argon atmosphere and the flask was heated to 150° C. using a hot gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl)methoxy)carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through celite and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl (S)-2-((((9H-fluoren-9-yl)methoxy)carbonyl)amino)-3-(isoquinolin-6-yl)propanoate (2.0 g, 66%) was obtained as a yellow gum. Analysis condition B: Retention time=2.46 min; ESI-MS(+) m/z [M+H]⁺: 495.3.

### Step2.

The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(isoquinolin-6-yl)propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl)methoxy)carbonyl)amino)-3-(isoquinolin-4-yl)propanoic acid

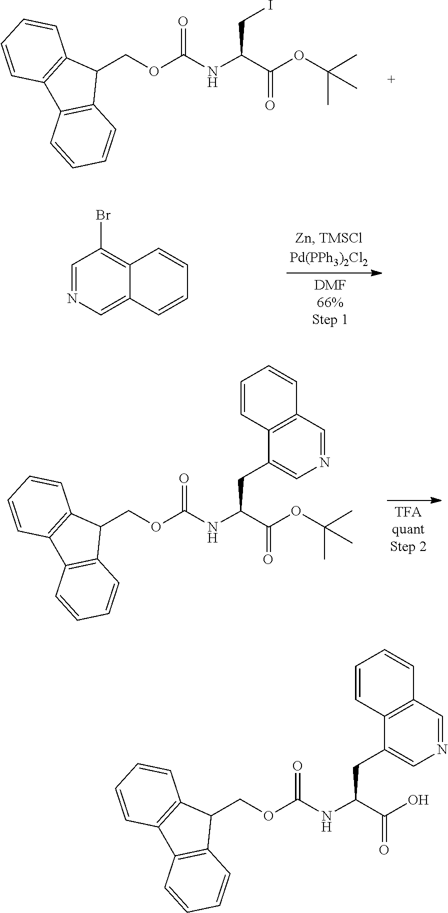 [SMILES: CC(C)(C)OC(=O)[C@H](CI)NC(=O)OCC1c2ccccc2-c2ccccc21 ; Brc1cncc2ccccc12 ; CC(C)(C)OC(=O)[C@H](Cc1cncc2ccccc12)NC(=O)OCC1c2ccccc2-c2ccccc21 ; O=C(N[C@@H](Cc1cncc2ccccc12)C(=O)O)OCC1c2ccccc2-c2ccccc21]

### Step 1.

To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was for 10 min. (R)-tert-butyl 2-((((9H-fluoren-9-yl)methoxy)carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone)dipalladium(0) (0.23 g, 0.25 mmol) and 4-bromoisoquinoline (2.11 g, 10.14 mmol) were added sequentially and the reaction was heated to 50° C. for 16 h. The reaction mixture was cooled to rt and treated with saturated ammonium chloride solution (200 mL). The crude was diluted with the ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl (S)-2-((((9H-fluoren-9-yl)methoxy)carbonyl)amino)-3-(isoquinolin-4-yl)propanoate (2.5 g, 50%).

Analysis condition E: Retention time=3.44 min; ESI-MS(+) m/z [M+H]⁺: 495.2.

### Step 2.

The final product was obtained following the same procedure of (S)-2-((((9H-fluoren-9-yl)methoxy)carbonyl)amino)-3-(2-methyl-1H-indol-3-yl)propanoic acid. TFA hydrolysis afforded the desired (S)-2-((((9H-fluoren-9-yl)methoxy)carbonyl)amino)-3-(isoquinolin-4-yl)propanoic acid as an off white solid in quantitative yield after purification diethyl ether trituration. ¹H NMR (400 MHz, DMSO-d₆) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl)methoxy)carbonyl)amino)-3-(4-(tert-butoxy)-3, 5-difluorophenyl)propanoic acid

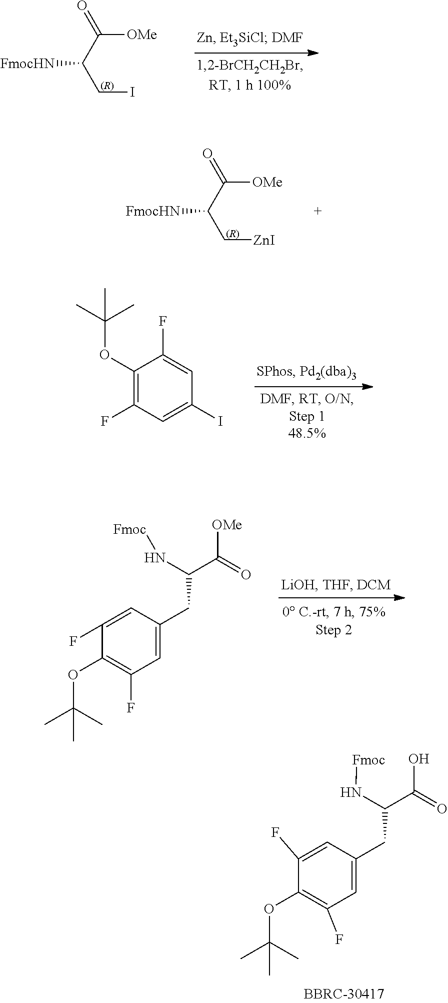

### Step 1.

The compound was prepared following the same procedure of tert-butyl (S)-2-((((9H-fluoren-9-yl)methoxy)carbonyl)amino)-3-(isoquinolin-4-yl)propanoate. First Negishi coupling with methyl (R)-2-((((9H-fluoren-9-yl)methoxy)carbonyl)amino)-3-iodopropanoate at 50° C. afforded the desired methyl (S)-2-((((9H-fluoren-9-yl)methoxy)carbonyl)amino)-3-(4-(tert-butoxy)-2,6-difluorophenyl)propanoate (5.5 g, 48.5% yield) after purification by flash chromatography.

Analysis condition E: Retention time=3.99 min; ESI-MS(+) m/z [M+NH₄]⁺: 527.2.

### Step 2.

In a multi-neck round bottom flask methyl (S)-2-((((9H-fluoren-9-yl)methoxy)carbonyl)amino)-3-(4-(tert-butoxy)-3,5-difluorophenyl)propanoate (11 g, 21.59 mmol) was added followed by the addition of tetrahydrofuran (132 mL) under nitrogen atmosphere at RT. The reaction mixture was cooled to 0° C. and LiOH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g redisep column, the product was eluted with 20% of ethyl acetate in petroleum ether. The reactions were concentrated to give (S)-2-((((9H-fluoren-9-yl)methoxy)carbonyl)amino)-3-(4-(tert-butoxy)-3,5-difluorophenyl)propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl3, 400 MHz) d 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of(S)-2-((((9H-fluoren-9-yl)methoxy)carbonyl)amino)-3-(isoquinolin-8-yl)propanoic acid

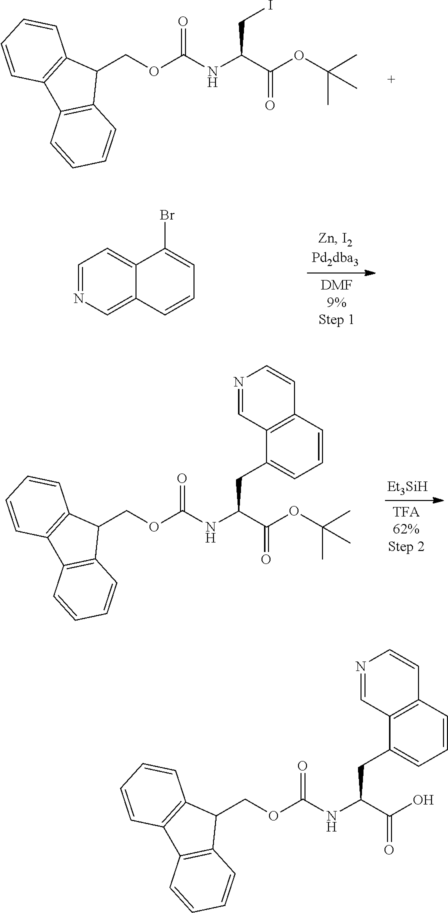

### Step 1.

Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl)methoxy)carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50 C for 4 h. under a positive pressure of nitrogen. The reaction mixture was cooled to rt, diluted with EtOAc (200 mL) and passed through Celite. The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford (S)-tert-butyl 2-((((9H-fluoren-9-yl)methoxy)carbonyl)amino)-3-(isoquinolin-8-yl)propanoate (380 mg, 0.768 mmol, 19.21% yield). Analysis condition G: Retention time=2.59 min; ESI-MS(+) m/z [M+H]⁺: 495.3.

### Step2.

(S)-tert-Butyl 2-((((9H-fluoren-9-yl)methoxy)carbonyl)amino)-3-(isoquinolin-8-yl)propanoate (380 mg, 0.768 mmol) was placed in 50-ml round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl)methoxy)carbonyl)amino)-3-(isoquinolin-8-yl)propanoic acid (320 mg, 0.712 mmol, 93% yield) as an off white solid. ¹H-NMR: (400 MHz, DMSO-d6) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]: 439.2 with 97.5% purity.

### Preparation of (S)-2-((((9H-fluoren-9-yl)methoxy)carbonyl)amino)-3-(7-fluoro-1H-indol-3-yl)propanoic acid

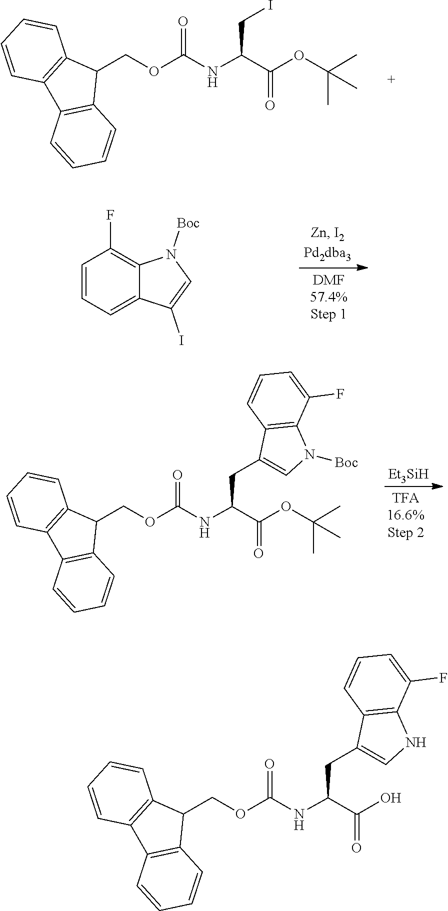

Step 1. Synthesis of tert-butyl 6-fluoro-3-iodo-1H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68 (s 1H), 7.30-7.34 (m, 1H), 7.03-7.08 (m, 1H), 1.66 (s, 9H) Step 2.

Compound was prepared following the same procedure of (S)-tert-butyl 2-((((9H-fluoren-9-yl)methoxy)carbonyl)amino)-3-(isoquinolin-8-yl)propanoate. First Negishi coupling at 50° C. afforded the desired tert-butyl (S)-3-(2-((((9H-fluoren-9-yl)methoxy)carbonyl)amino)-3-(tert-butoxy)-3-oxopropyl)-7-fluoro-1H-indole-1-carboxylate (690 mg, 1.149 mmol, 57.4% yield) after purification by flash chromatography.

Analysis condition H: Retention time=3.885 min; ESI-MS(+) m/z [M−Boc−tBu+H]⁺: 445.2.

### Step 3

Final product was obtained following the same procedure of (S)-2-((((9H-fluoren-9-yl)methoxy)carbonyl)amino)-3-(isoquinolin-8-yl)propanoic acid. TFA hydrolysis afforded the desired (S)-2-((((9H-fluoren-9-yl)methoxy)carbonyl)amino)-3-(7-fluoro-1H-indol-3-yl)propanoic acid as an off white powder (96 mg, 0.191 mmol, 16.63% yield) after purification by reverse phase prep HPLC (Column: 80 g size, Silisep C18, 19×150 mm, 5 μm, Mobile phases: A=10 mM ammonium acetate in water, B=MeoH. 15 mL/min flow Gradient: 0-20 min, 5-30% B, 20-55 min, 30-80% B, 55-60 min, 80-100% B, held at 100% B for 5 min. Compound was eluted at 75% B) followed by lyophilization.

Analysis condition F: Retention time=1.367 min; ESI-MS(+) m/z [M+H]⁺: 445.3. ¹H-NMR (400 MHz, DMSO-d6) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3S)-2-((((9H-fluoren-9-yl)methoxy)carbonyl)amino)-3-(1-(tert-butoxycarbonyl)-1H-indol-3-yl)butanoic acid

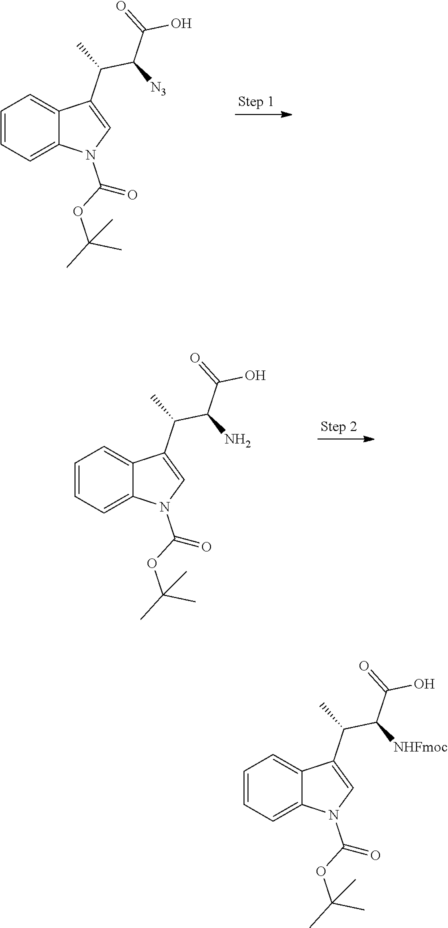

Compound (2S,3S)-2-azido-3-(1-(tert-butoxycarbonyl)-1H-indol-3-yl)butanoic acid was prepared following the procedure reported in Tetrahedron Letters 2001, 42, 4601-4603. The azide reduction step used different conditions as detailed below.

### Step 1.

To a solution of (2S,3S)-2-azido-3-(1-(tert-butoxycarbonyl)-1H-indol-3-yl)butanoic acid (1000 mg, 2.90 mmol) in THE (58 mL) was added platinum(IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through Celite©. The solvent was removed under vacuum and the crude residue was redissolved in EtOH. This solution was filtered through Celite© to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

### Step 2.

To a solution of (2S,3S)-2-amino-3-(1-(tert-butoxycarbonyl)-1H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et₃N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d₆) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H). Analysis condition B: Retention time=1.27 min; ESI-MS(+) m/z [M+H]⁺: not observed.

### Preparation of (S)-2-((((9H-fluoren-9-yl)methoxy)carbonyl)amino)-3-(6-(o-tolyl)pyridin-3-yl)propanoic acid

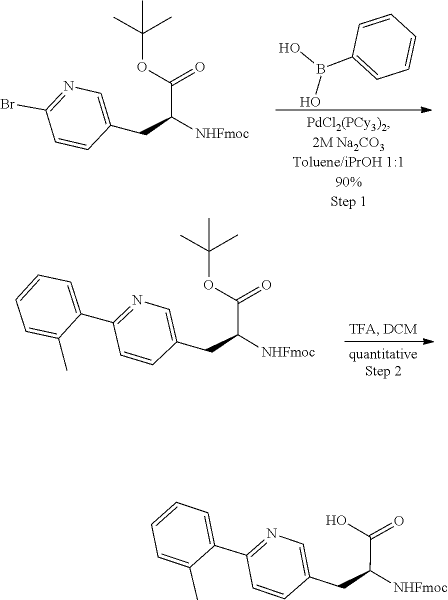

### Step 1.

To a stirred solution of tert-butyl (S)-2-((((9H-fluoren-9-yl)methoxy)carbonyl)amino)-3-(6-bromopyridin-3-yl)propanoate (1750 mg, 3.35 mmol) in toluene/iPrOH (1:1, v:v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine)palladium(II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc:DCM (1:9) as eluant lead to tert-butyl (S)-2-((((9H-fluoren-9-yl)methoxy)carbonyl)amino)-3-(6-(o-tolyl)pyridin-3-yl)propanoate (1.81 g, 3.39 mmol, 90%) as a colorless oil.

### Step 2.

(S)-2-((((9H-Fluoren-9-yl)methoxy)carbonyl)amino)-3-(6-(o-tolyl)pyridin-3-yl)propanoate (1750 mg, 3.19 mmol) was dissolved in trifluoroacetic acid (5.00 mL) and the reaction was allowed to stir at room temperature for two hours. The reaction was brought to dryness on rotovap and the crude product was dissolved in diethyl ether and 1M HCl in diethyl ether. The mixture was sonicated for 2 hours to give a white solid. The product was isolated by filtration and washed with water to give (S)-2-((((9H-fluoren-9-yl)methoxy)carbonyl)amino)-3-(6-(o-tolyl)pyridin-3-yl)propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl)methoxy)carbonyl)amino)-3-(4′-acetamido-[1,1′-biphenyl]-4-yl)propanoic acid

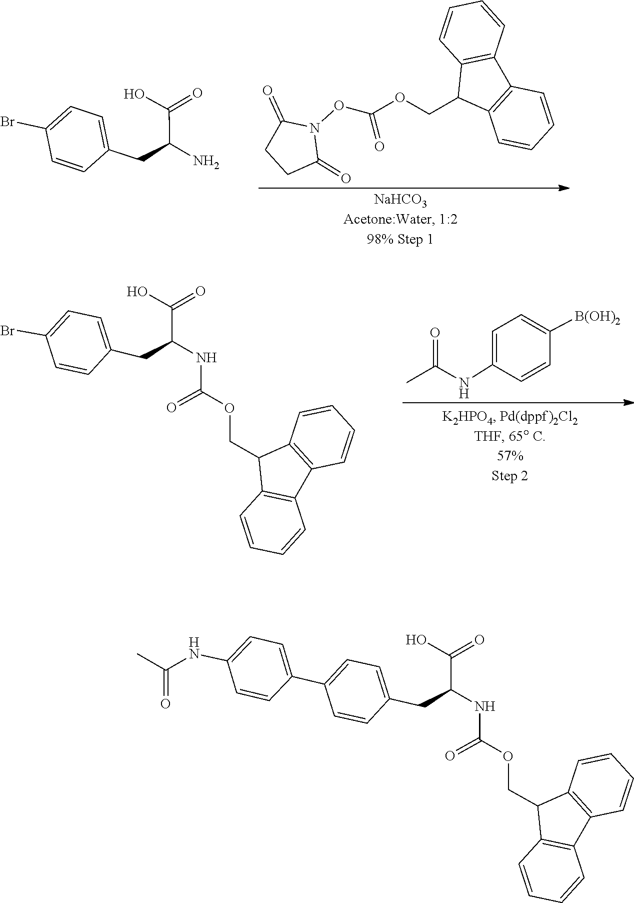 [SMILES: N[C@@H](Cc1ccc(Br)cc1)C(=O)O ; O=C(OCC1c2ccccc2-c2ccccc21)ON1C(=O)CCC1=O ; O=C(N[C@@H](Cc1ccc(Br)cc1)C(=O)O)OCC1c2ccccc2-c2ccccc21 ; CC(=O)Nc1ccc(B(O)O)cc1 ; CC(=O)Nc1ccc(-c2ccc(C[C@H](NC(=O)OCC3c4ccccc4-c4ccccc43)C(=O)O)cc2)cc1]

### Step 1.

A 5.0-1 multi-neck round-bottomed flask was charged with (S)-2-amino-3-(4-bromophenyl)propanoic acid (150.0 g, 615 mmol), Fmoc-OSu (207 g, 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

### Step 2.

To a stirred solution of (S)-2-((((9H-fluoren-9-yl)methoxy)carbonyl)amino)-3-(4-bromophenyl)propanoic acid (1.0 g, 2.144 mmol) and (4-acetamidophenyl)boronic acid (0.576 g, 3.22 mmol) with THE (50 mL) in 150-ml pressure tube, Argon was purged for 5 min. Potassium phosphate, tribasic (1.366 g, 6.43 mmol) was then added and the purging was continued for another 5 min. 1,1′-bis(di-tert-butylphosphino)ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to get the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d₆) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

### Synthesis of Aryl/Heteroaryl Substituted Phenylalanines

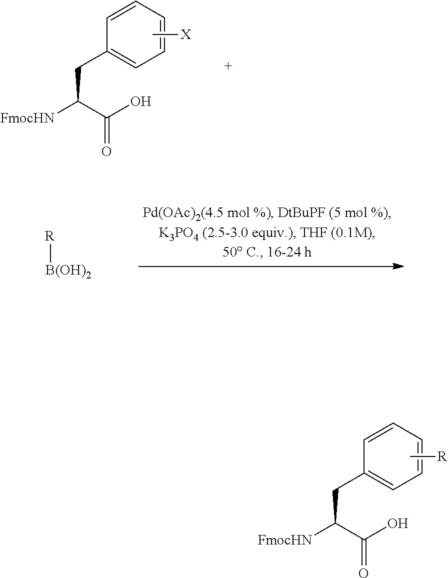

### General Procedures for Suzuki-Miyaura Coupling (SMC) Reactions in Scheme 1.

To a N₂-flushed 20-mL scintillation vial equipped with a magnetic stir bar was added Fmoc-halo-Phe-OH (0.5 mmol), boronic acid (1.5-2.5 equiv.), and anhydrous THE (6 mL). The suspension was degassed by bubbling N₂ into the vial for several minutes. Palladium(II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl)propanoic acid

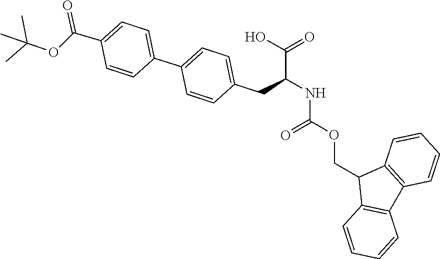 [SMILES: CC(C)(C)OC(=O)c1ccc(-c2ccc(C[C@H](NC(=O)OCC3c4ccccc4-c4ccccc43)C(=O)O)cc2)cc1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl)methoxy)carbonyl)amino)-3-(3′-(tert-butoxycarbonyl)-[1,1′-biphenyl]-4-yl)propanoic acid

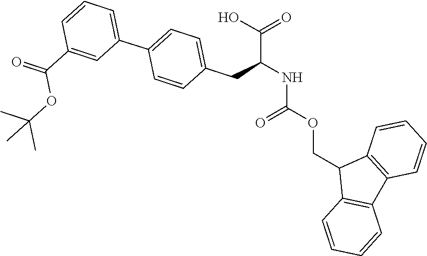 [SMILES: CC(C)(C)OC(=O)c1cccc(-c2ccc(C[C@H](NC(=O)OCC3c4ccccc4-c4ccccc43)C(=O)O)cc2)c1]

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 85% (240 mg); off-white solids. ¹H NMR (500 MHz, DMSO-d₆) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁺ 581.26461, found at 581.26474, mass difference 0.218 ppm.

### Preparation of (S)-2-((((9H-fluoren-9-yl)methoxy)carbonyl)amino)-3-(4-boronophenyl)propanoic acid (ELN: A0934-595-01)

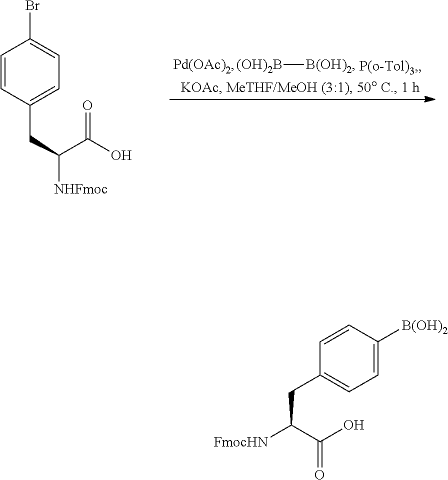

To a 75-ml pressure bottle (S)-2-((((9H-fluoren-9-yl)methoxy)carbonyl)amino)-3-(4-bromophenyl)propanoic acid (6.0 g, 12.87 mmol) and 2-methyl THE (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol)were added every in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through celite, and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through celite, concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl)methoxy)carbonyl)amino)-3-(4-boronophenyl)propanoic acid (4.0 g, 9.28 mmol, 72.1% yield) as a white solid. LCMS: 432.1 (M+H), tr=0.82 min. ¹H NMR (500 MHz, DMSO-d₆) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl)methoxy)carbonyl)amino)-3-(4′-fluoro-[1,1′-biphenyl]-4-yl)propanoic acid

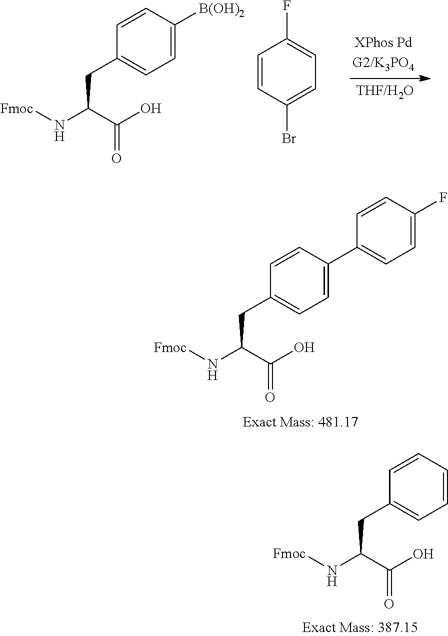

To a stirred solution of (S)-2-((((9H-fluoren-9-yl)methoxy)carbonyl)amino)-3-(4-boronophenyl)propanoic acid (217.5 mg, 0.504 mmol), 1-bromo-4-fluorobenzene (0.083 mL, 0.757 mmol) and XPhos Pd G2 (9.7 mg, 0.012 mmol) in THE (1 mL) at rt was added 0.5 M aqueous K₃PO₄ (2 mL, 1.000 mmol). N₂ was purged with vacuum three times and the mixture was stirred at 80° C. for 16 h. The mixture was cooled to rt. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash Rf, gradient of 0% to 20% MeOH/CH₂Cl₂ over 15 column volumes, RediSep SiO₂ 40 g). Fractions containing the desired product were collected and concentrated to give (S)-2-((((9H-fluoren-9-yl)methoxy)carbonyl)amino)-3-(4′-fluoro-[,1′-biphenyl]-4-yl)propanoic acid (206.1 mg, 0.43 mmol, 85% yield) as a cream solid: HPLC: RT=1.04 min (Waters Acquity UPLC BEH C18 1.7 um 2.1×50 mm, CH₃CN/H₂O/0.05% TFA, 1 min. gradient, wavelength=254 nm); MS (ES): m/z=482 [M+H]⁺. ¹H NMR (499 MHz, DMSO-d₆) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl)methoxy)carbonyl)amino)-3-(3′,5′-difluoro-[1,1′-biphenyl]-4-yl)propanoic acid

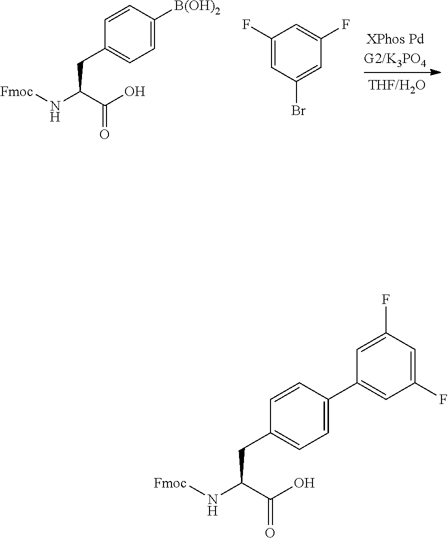

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′, 5′-difluoro-[1,1′-biphenyl]-4-yl)propanoic acid (197.1 mg, 0.40 mmol, 78% yield) as a colorless solid after purification by flash chromatography. HPLC: RT=1.06 min (Waters Acquity UPLC BEH C18 1.7 um 2.1×50 mm, CH₃CN/H₂O/0.05% TFA, 1 min. gradient, wavelength=254 nm); MS (ES): m/z=500 [M+H]⁺. ¹H NMR (499 MHz, DMSO-d₆) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl)methoxy)carbonyl)amino)-3-(3′,4′,5′-trifluoro-[1,1′-biphenyl]-4-yl)propanoic acid

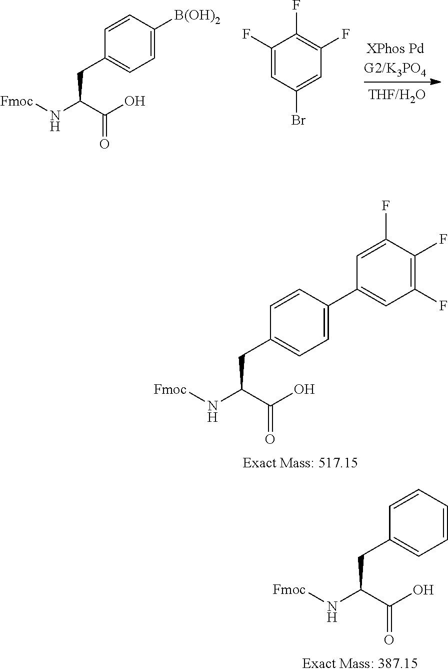

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,4′,5′-trifluoro-[1,1′-biphenyl]-4-yl)propanoic acid (218.5 mg, 0.422 mmol, 84% yield) as a colourless solid after purification by flash chromatography. HPLC: RT=1.466 min (Shimadzu UPLC with Waters Acquity BEH C18 1.7 um 2.1×50 mm column, CH₃CN/H₂O/0.1% TFA, 3 min. gradient, wavelength=254 nm); MS (ES): m/z=556. ¹H NMR (499 MHz, DMSO-d₆) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

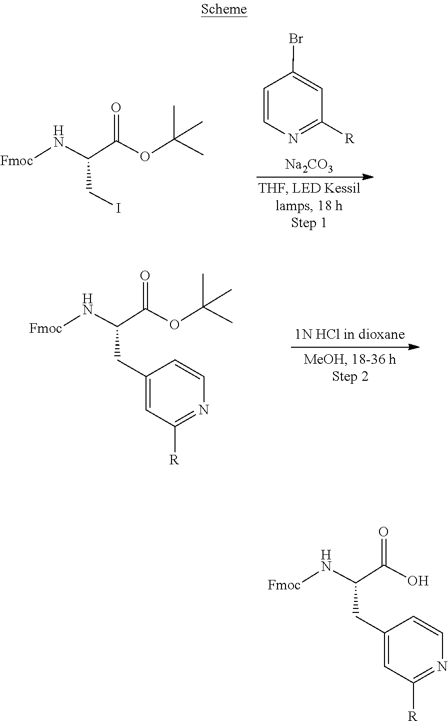

General procedure for photoredox reaction.

Ir[dF(CF₃)ppy₂]₂(dtbbpy)PF₆ (0.018 g, 0.016 mmol, 1 mol %), tert-butyl (R)-2-((((9H-fluoren-9-yl)methoxy)carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane)silane (0.278 g, 1.596 mmol, 1.00 equiv) were charged into an oven-dried 40-mL pressure-relief screw cap vial. The vial was capped, purged with nitrogen, diluted with THE (45.0 mL), and then sonicated. In a separate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complex solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on rotovap. The crude product was dissolved in a minimum amount of methanol and dry loaded on silica gel column for purification.

### Preparation of (2S)-2-({[(9H-fluoren-9-yl)methoxy]carbonyl}amino)-3-(2-methoxypyridin-4-yl)propanoic acid

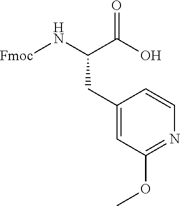

The mixture was rotovaped onto silica gel, purified by isco using 10% to 80% EtOAc/Hexanes.

The fractions were pooled, concentrated to obtain the desired product as a clear oil (237 mg, 100%)

Analysis conditions D: Retention time 1.74 min; ES+ 475.1.

### Preparation of ((S)-2-((((9H-fluoren-9-yl)methoxy)carbonyl)amino)-3-(4-(trifluoromethoxy)phenyl)propanoic acid

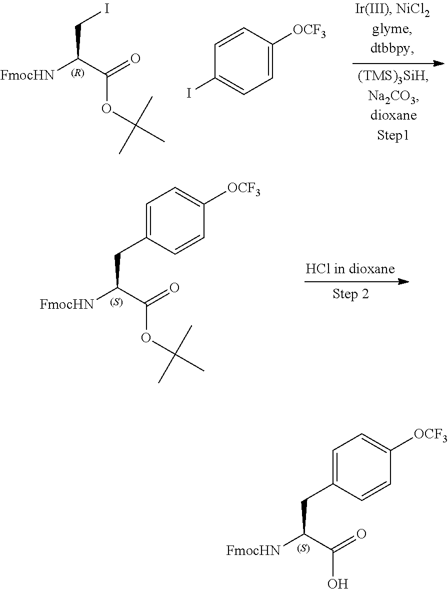

### Step 1.

In 4 separate 40-mi vials was placed Ir(dF(CF₃)ppy)₂(dtbbpy)PF₆ (5.6 mg, 4.99 μmol) and Na₂CO₃ (249 mg, 2.35 mmol) in dioxane (18 mL), and was fitted with a teflon screw cap and a stir bar. To the mixture was added 1-iodo-4-(trifluoromethoxy)benzene (0.16 mL, 1.02 mmol) stirred briefly, then tris(trimethylsilyl)silane (0.23 mL, 0.75 mmol) was added via syringe, and the suspension was degassed (cap on) with nitrogen for 5 min. To a separate 40-mL vial was added nickel(II) chloride ethylene glycol dimethyl ether complex (22 mg, 0.10 mmol) and 4,4′-di-tert-butyl-2,2′-bipyridine (33 mg, 0.12 mmol)ioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl)methoxy)carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, stirred for 40 h. The reactions were removed from the illumination/reactor. The blackish reaction mixtures of each vial were poured into a 500-ml erlenmeyer flask into which was added EtOAc (200 mL). The mixture was filtered through celite, washed with EtOAc, and concentrated. The residue was purified by flash chromatography (Teledyne ISCO CombiFlash Rf, gradient of 0% using solvent A/B=CH₂Cl₂/EtOAcover 10 column volumes, RediSep SiO2 80 g loaded as DCM solution). The fractions containing the desired product were collected and concentrated to obtained the product tert-butyl (S)-2-((((9H-fluoren-9-yl)methoxy)carbonyl)amino)-3-(4-(trifluoromethoxy)phenyl)propanoate (865.2 mg, 1.64 mmol, 82% yield, only about 73% HPLC purity as a colourless oil and was used as was in the deprotection step: HPLC: RT=1.62 min (Waters Acquity UPLC BEH C18 1.7 um 2.1×50 mm, CH₃CN/H₂O/0.05% TFA, 1 min. gradient, wavelength=254 nm); MS (ES): m/z=550 [M+23]+.

### Step 2.

To a stirred solution of tert-butyl (S)-2-((((9H-fluoren-9-yl)methoxy)carbonyl)amino)-3-(4-(trifluoromethoxy)phenyl)propanoate (865.2 mg, 1.64 mmol) in dichloromethane (8.2 mL) at rt was added HCl (4M in dioxane, 8.20 mL, 32.8 mmol). The mixture was stirred at rt for 18 h. The mixture was concentrated in vacuo then dried under vacuum. The residue was dissolved in DMF (4 mL), purified on ISCO ACCQ Prep over 2 injections. The fractions containing the desire product were combined and partially concentrated on rotovap, then blown air over mixture over weekend. The residue was dissolved in CH₃CN, diluted with water, frozen, and lyophilized. To obtained the product (S)-2-((((9H-fluoren-9-yl)methoxy)carbonyl)amino)-3-(4-(trifluoromethoxy)phenyl)propanoic acid (344.1 mg, 0.73 mmol, 44.5% yield) as a colorless solid. HPLC: RT=1.38 min (Waters Acquity UPLC BEH C18 1.7 um 2.1×50 mm, CH₃CN/H₂O/0.05% TFA, 1.5 min. gradient, wavelength=254 nm); MS (ES): m/z=472 [M+1]+.

¹H NMR (499 MHz, DMSO-d₆) ppm δ 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of (S)-2-((((9H-fluoren-9-yl)methoxy)carbonyl)amino)-3-(2,5-dimethylphenyl)propanoic acid

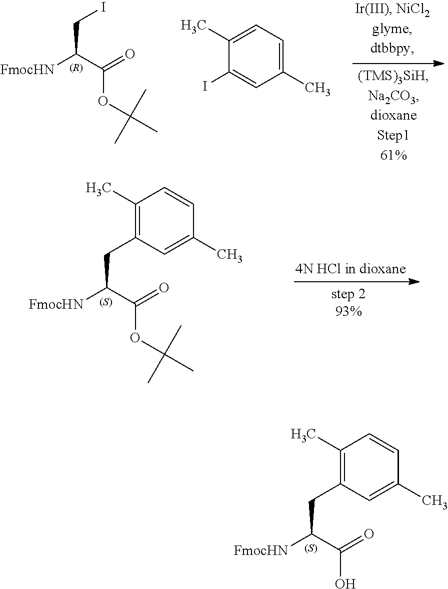

### Step 1.

Compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,5-dimethylphenyl)propanoate (140.5 mg, 0.298 mmol, 61.1% yield) after purification by flash chromatography. Analysis condition J: Retention time=1.21 min; ESI-MS(+) m/z [M-tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H).

### Step 2.

Final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,5-dimethylphenyl)propanoic acid (115.2 mg, 0.277 mmol, 93% yield) as a cream solid after purification by reverse phase flash chromatography. HPLC: RT=1.03 min (Waters Acquity UPLC BEH C18 1.7 um 2.1×50 mm, CH3CN/H2O/0.05% TFA, 1 min. gradient, wavelength=254 nm); MS (ES): m/z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H).

### Preparation of (S)-2-((((9H-fluoren-9-yl)methoxy)carbonyl)amino)-3-(4-fluoro-3-methylphenyl)propanoic acid

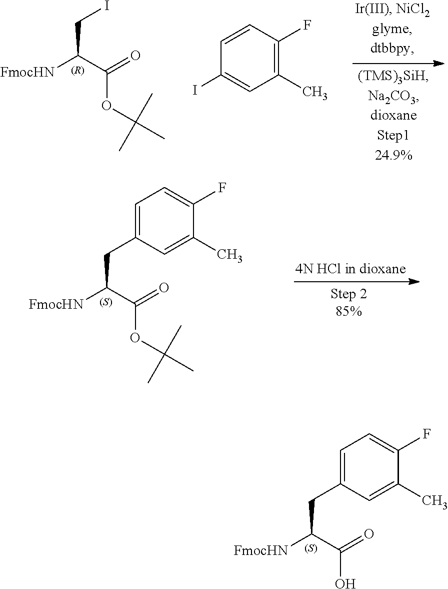

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(4-fluoro-3-(trifluoromethyl)phenyl)propanoate (66.3 mg, 0.13 mmol, 24.9% yield) as a colourless solid after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=474 [M-tBu]⁺. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H).

### Step 2.

Final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(4-fluoro-3-methylphenyl)propanoic acid (58.3 mg, 0.139 mmol, 85% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+H]⁺. ¹H NMR (499 MHz, DMSO-d₆) δ 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl)methoxy)carbonyl)amino)-3-(2,4-difluoro-5-methoxyphenyl)propanoic acid

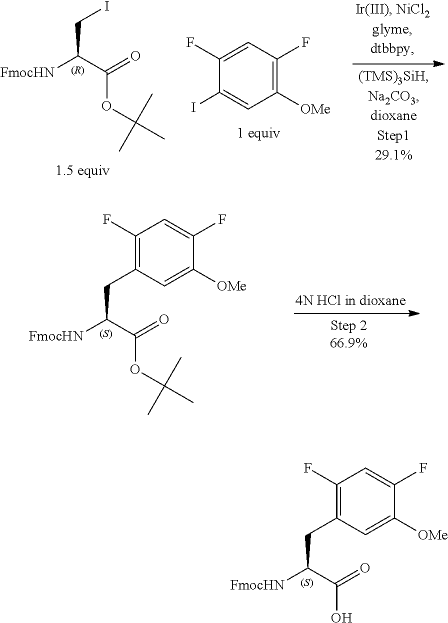

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,4-difluoro-5-methoxyphenyl)propanoate (77.1 mg, 0.151 mmol, 29.1% yield as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=454 [M-t-Bu]⁺. ¹H NMR (499 MHz, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,4-difluoro-5-methoxyphenyl)propanoic acid (45.9 mg, 0.101 mmol, 66.9% yield) as a cream solid after purification by reverse phase flash chromatography. HPLC: RT=0.99 min (Waters Acquity UPLC BEH C18 1.7 um 2.1×50 mm, CH₃CN/H₂O/0.05% TFA, 1 min. gradient, wavelength=254 nm); MS (ES): m/z=454 [M+1]⁺. ¹H NMR (499 MHz, DMSO-d₆) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H).

### Preparation of (S)-2-((((9H-fluoren-9-yl)methoxy)carbonyl)amino)-3-(2,3-dimethylphenyl)propanoic acid

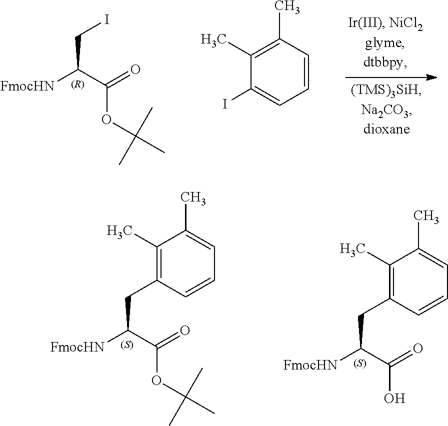

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,3-dimethylphenyl)propanoate (107.5 mg, 0.228 mmol, 55.5% yield) as a tan viscous oil after purification by flash chromatography. HPLC: RT=1.21 min (Waters Acquity UPLC BEH C18 1.7 um 2.1×50 mm, CH₃CN/H₂O/0.05% TFA, 1 min. gradient, wavelength=254 nm); MS (ES): m/z=416 [M-t-Bu]⁺. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,3-dimethylphenyl)propanoic acid (72.9 mg, 0.175 mmol, 77% yield) as a cream solid after purification by reverse phase flash chromatography. HPLC: RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]⁺. ¹H NMR (499 MHz, DMSO-d₆) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of (S)-2-((((9H-fluoren-9-yl)methoxy)carbonyl)amino)-3-(2-fluoro-3-methylphenyl)propanoic acid

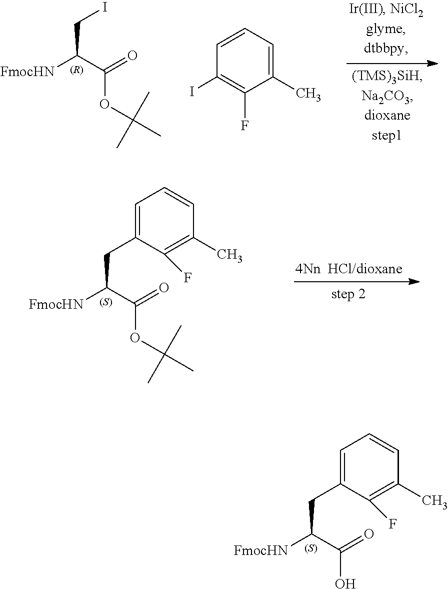

### Step 1

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-3-methylphenyl)propanoate (136.9 mg, LCMS showed 77% product and 23% impurity) as a viscous oil after purification by flash chromatography. Used as is, purify at after tBu hydrolysis.

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-3-methylphenyl)propanoic acid (79.7 mg, 0.190 mmol, 66.0% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.79 (br s, 1H), 7.89 (d, J=7.7 Hz, 2H), 7.78 (d, J=8.6 Hz, 1H), 7.65 (dd, J=11.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). ¹H NMR and LCMS showed a 14% impurity.

### Preparation of ((S)-2-((((9H-fluoren-9-yl)methoxy)carbonyl)amino)-3-(2-fluoro-5-methylphenyl)propanoic acid

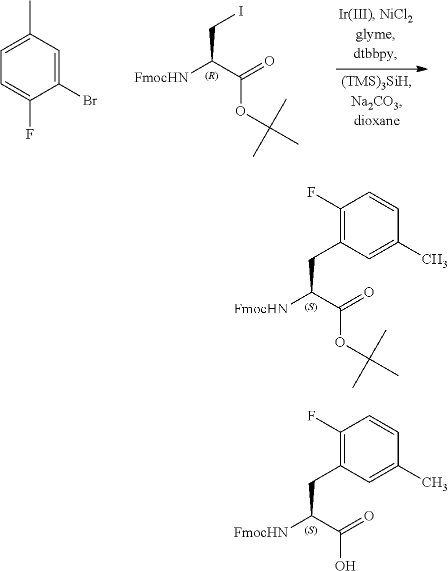

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-5-methylphenyl)propanoate (148.1 mg, 0.311 mmol, 65.4% yield) as a colourless gum after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=420 [M-t-Bu]⁺. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methylphenyl)propanoic acid (98.1 mg, 0.23 mmol, 75% yield) as a colourless solid after purification by reverse phase flash chromatography. HPLC: RT=1.01 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H).

### Preparation of (S)-2-((((9H-fluoren-9-yl)methoxy)carbonyl)amino)-3-(2-fluoro-5-methoxyphenyl)propanoic acid

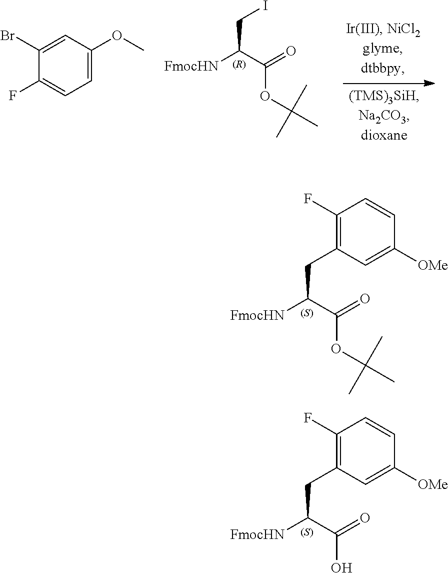

### Step 1

The compound was prepared following the same procedure of tert-butyl (S)-2-((((9H-fluoren-9-yl)methoxy)carbonyl)amino)-3-(2-fluoro-5-methoxyphenyl)propanoate (117.7 mg, 0.24 mmol, 50.4% yield) as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=436 [M-t-Bu]*. H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H).

### Step 2

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-fluoro-5-methoxyphenyl)propanoic acid (79.5 mg, 0.183 mmol, 76% yield) as a colourless solid after purification by flash chromatography. HPLC: RT=0.98 min (Waters Acquity UPLC BEH C18 1.7 um 2.1×50 mm, CH₃CN/H₂O/0.05% TFA, 1 min. gradient, wavelength=254 nm); MS (ES): m/z=436 [M+1]⁺. Base peak of 214=fully deprotected amino acid fragment was also observed. ¹H NMR (499 MHz, DMSO-d₆) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl)methoxy)carbonyl)amino)-3-(2-methoxy-5-methylphenyl)propanoic acid

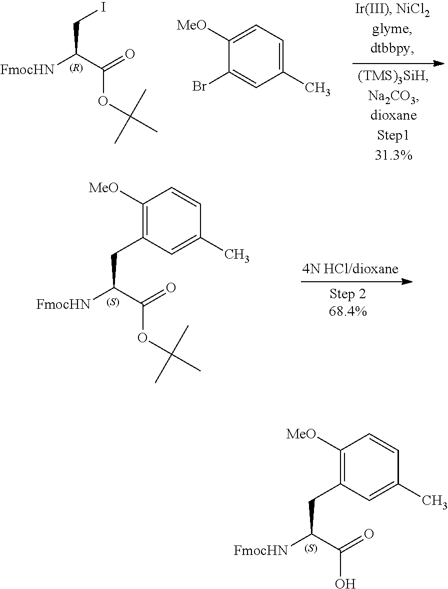

### Step 1.

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-methoxy-5-methylphenyl)propanoate (73.9 mg, 0.15 mmol, 31.3% yield) as a colourless film after purification by flash chromatography. HPLC: RT=1.20 min (Waters Acquity UPLC BEH C18 1.7 um 2.1×50 mm, CH₃CN/H₂O/0.05% TFA, 1 min. gradient, wavelength=254 nm); MS (ES): m/z=488 [M-tBu+H]⁺. ¹H NMR (499 MHz, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H).

### Step 2.

The final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2-methoxy-5-methylphenyl)propanoic acid (44.7 mg, 0.104 mmol, 68.4% yield) as a colourless solid after purification by flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=432 [M+H]⁺. ¹H NMR (499 MHz, DMSO-d₆) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J=13.5, 4.8 Hz, 1H), 2.72 (dd, J=13.4, 10.2 Hz, 1H), 2.16 (s, 3H).

### Preparation of(S)-2-((((9H-fluoren-9-yl)methoxy)carbonyl)amino)-3-hydroxy-3-methylbutanoic acid

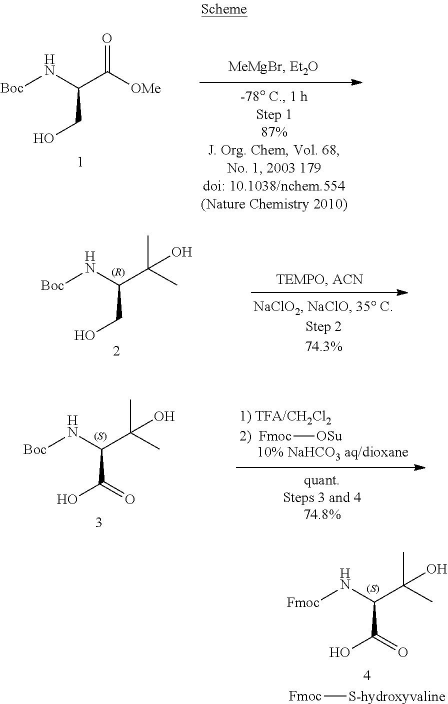

### Step 1.

To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by I2PAC. Desired fractions were eluted at 50% EtOAc:petroleum ether mixture, and were collected and concentrated at 40° C. to give tert-butyl (R)-(1,3-dihydroxy-3-methylbutan-2-yl)carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

### Step 2.

A 50-ml single neck round-bottomed flask was charged with tert-butyl (R)-(1,3-dihydroxy-3-methylbutan-2-yl)carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and the pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude (S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9s, 3H).

### Step 3.

A 2000-mL single neck flask was charged with (S)-2-((tert-butoxycarbonyl)amino)-3-hydroxy-3-methylbutanoic acid (90 g, 386 mmol)dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude (S)-2-amino-3-hydroxy-3-methylbutanoic acid, HCl (70 g, 107% yield) as a white solid and was taken directly to the next step.

### Step 4.

To a 3000-ml multi-neck round-bottomed flask was charged (S)-2-amino-3-hydroxy-3-methylbutanoic acid, HCl (70 g, 413 mmol), dioxane (1160 mL) and water (540 mL) The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following procedure was followed: the solid was dissolved in ethyl acetate (1200 mL) and was stripped off with n-hexane (3000 mL). The slurry obtained was stirred for 10 min, filtered, dried under vacuum to give (S)-2-((((9H-fluoren-9-yl)methoxy)carbonyl)amino)-3-hydroxy-3-methylbutanoic acid (112.0 g, 74.8 yield for two steps) as a white solid.

### Preparation of (S)-2-((((9H-fluoren-9-yl)methoxy)carbonyl)amino)-3-(3,4,5-trifluorophenyl)propanoic acid

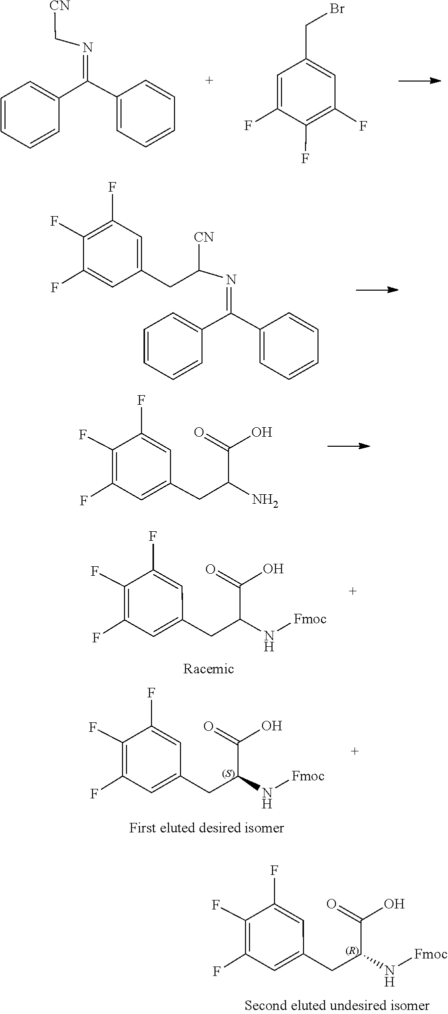

### Step 1.

To a stirred solution of 2-((diphenylmethylene)amino)acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at rt overnight. After 26 h, the reaction mixture was diluted with water (500 mL) and the DCM layer was separated. The aqueous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl)propanenitrile (140 g, 384 mmol, 85% yield) as a yellow solid. Analysis condition E: Retention time=3.78 min; ESI-MS(+) m/z [M+H]⁺: 365.2.

### Step 2.

To a stirred solution of 2-((diphenylmethylene)amino)-3-(3,4,5-trifluorophenyl)propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for next step.

### Step 3.

To the crude aqueous dioxane solution from the previous was added 10 N NaOH solution until the solution was neutral. Na₂CO₃ (438 ml, 438 mmol) was then added, followed by the addition of Fmoc-OSu (81 g, 241 mmol). The mixture was stirred at rt overnight. The aqueous solution was acidified with 1.5 N HCl till pH=2 and the solid formed was filtered, dried to afford the crude compound. It was slurried initially with 5% EtOAc/petroleum ether for 30 min and filtered. The filtered compound was further slurried with ethyl acetate for 20 min and filtered to get the crude racemic 2-((((9H-fluoren-9-yl)methoxy)carbonyl)amino)-3-(3,4,5-trifluorophenyl)propanoic acid (90 g, 204 mmol, 93% yield) as an off-white solid. This racemic compound was separated into two isomers by SFC purification to get the desired isomers. After concentration of the desired isomer, it was slurried with 5% EtOAc/petroleum ether and filtered to get (S)-2-((((9H-fluoren-9-yl)methoxy)carbonyl)amino)-3-(3,4,5-trifluorophenyl)propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ −137.56 (d, J=19.6 Hz, 2F), −166.67 (t, J=19.6 Hz, 1F). Analysis condition E: Retention time=3.15 min; ESI-MS(+) m/z [M+H]⁺: 442.2.

The other fraction was concentrated to get (R)-2-((((9H-fluoren-9-yl)methoxy)carbonyl)amino)-3-(3,4,5-trifluorophenyl)propanoic acid (40 g, 91 mmol, 41.4% yield) as an off-white solid.

### Preparation of (S)-2-((((9H-fluoren-9-yl)methoxy)carbonyl)amino)-4-(tert-butoxy)-3,3-dimethyl-4-oxobutanoic acid

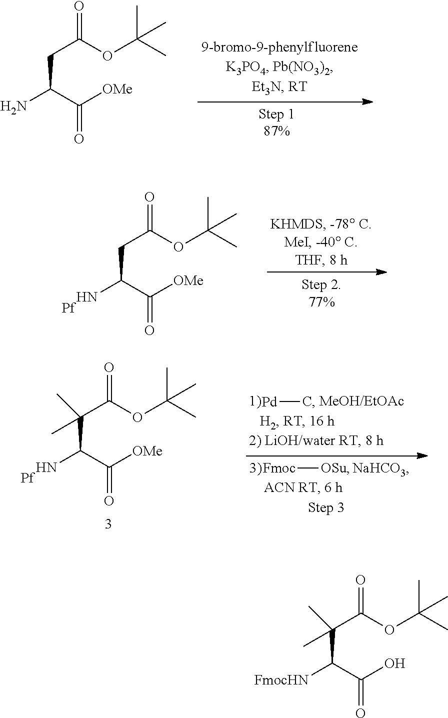

### Step1.

To a stirred solution of 4-(tert-butyl) 1-methyl L-aspartate, HCl salt (34 g, 142 mmol) in acetonitrile (550 mL), was added lead(II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% EA in PE) and LCMS. The reaction mixture was filtered over Celite, washed with chloroform, and evaporated to get thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to get pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]⁺: 466.40.

### Step 2.

A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃(200 mL), and brine. The organic layer was dried over anhydrous Na₂SO₄, evaporated in vacuo, and recrystallized from hexanes to give 1-(tert-butyl) 4-methyl (S)-2,2-dimethyl-3-((9-phenyl-9H-fluoren-9-yl)amino)succinate (18.5 g, 39.2 mmol, 77% yield) as a white solid, which was taken for next step. Analysis condition L: Retention time=2.04 min; ESI-MS(+) m/z [M+Na]⁺: 494.34.

### Step3.

A stirred solution of 1-(tert-butyl) 4-methyl (S)-2,2-dimethyl-3-((9-phenyl-9H-fluoren-9-yl)amino)succinate (24 g, 50.9 mmol) in methanol (270 mL) and ethyl acetate (100 mL) was degassed with nitrogen. Pd—C (2.71 g, 2.54 mmol) (10% by weight) was added, and the mixture was flushed with hydrogen gas and then stirred at RT in 1-liter capacity autoclave with 50 psi overnight. The reaction mixture was filtered through celite pad, washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

### Step 4.

To a stirred solution of 1-(tert-butyl) 4-methyl (S)-3-amino-2,2-dimethylsuccinate (11.0 g, 47.6 mmol)cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% EA in petroleum ether to get (S)-2-((((9H-fluoren-9-yl)methoxy)carbonyl)amino)-4-(tert-butoxy)-3, 3-dimethyl-4-oxobutanoic acid (12.2 g, 26.9 mmol, 39.0% yield) as a white solid. ¹HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of (S)-2-((((9H-fluoren-9-yl)methoxy)carbonyl)amino)-3-(3-(tert-butoxycarbonyl)phenyl)propanoic acid

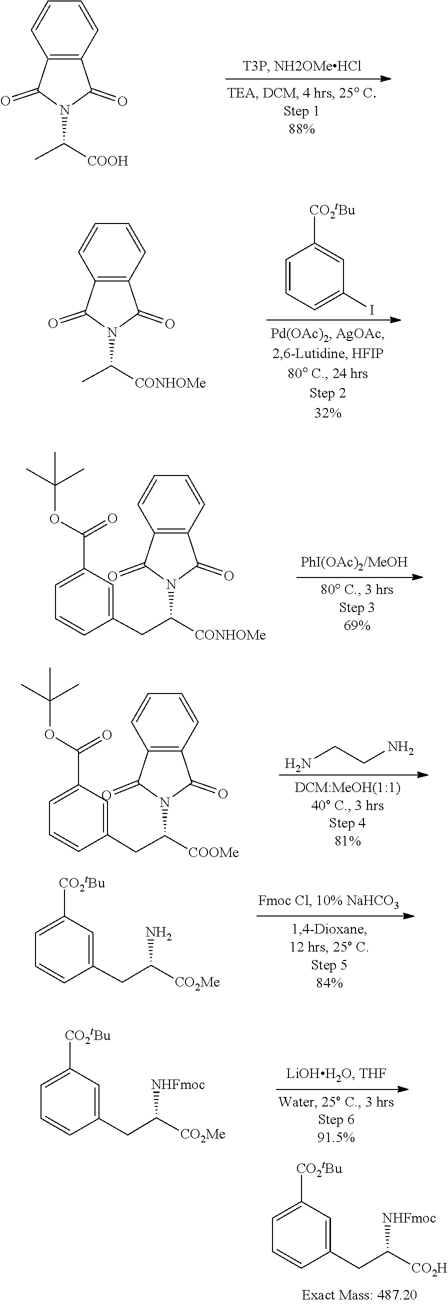

### Step 1.

To a solution of (S)-2-(1,3-dioxoisoindolin-2-yl)propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using 120 g silica column with 38 to 45% EtOAc in petroleum ether to give (S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H).

### Step 2.

To a solution of (S)-2-(1,3-dioxoisoindolin-2-yl)-N-methoxypropanamide (20 g, 81 mmol), palladium(II) acetate (1.809 g, 8.06 mmol), silver acetate (26.9 g, 161 mmol) placed in a 1000-ml seal tube, was added tert-butyl 3-iodobenzoate (36.8 g, 121 mmol), 2,6-Lutidine (2.395 ml, 24.17 mmol), HFIP (300 ml) at 25° C. under N₂ atmosphere. The reaction was stirred for 15 min at 25° C. under N₂ and then heated Up to 80° C. for 24 h with vigorous stirring. The reaction mixture was filtered through celite and washed with DCM (200 mL). The combined organic layer was concentrated under reduced pressure. The crude product was purified via combiflash using 220 g silica column eluting with 25 to 30% EtOAc:CHCl₃ to obtain the desired product tert-butyl (S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H).

### Step 3.

To a solution of tert-butyl (S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The Reaction was concentrated under reduced pressure to get the crude product. It was purified with silica gel chromatography (100-200 mesh eluting with 20% EA: hexane) to obtain the desired compound tert-butyl (S)-3-(2-(1,3-dioxoisoindolin-2-yl)-3-methoxy-3-oxopropyl)benzoate (10 g, 24.42 mmol, 69.1% yield. ¹H NMR (CDCl3, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H).

### Step 4.

To a solution of tert-butyl (S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% EA: hexane) to obtain the desired compound tert-butyl (S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H).

### Step 5.

To a solution of tert-butyl (S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetylate. The organic layer was concentrated under reduced pressure to get the crude product. It was purified via silica gel chromatography (100-200 mesh eluting with 20% EA: hexane) to obtain the desired compound tert-butyl (S)-3-(2-((((9H-fluoren-9-yl)methoxy)carbonyl)amino)-3-methoxy-3-oxopropyl)benzoate (15 g, 29.9 mmol, 84% yield).

### Step 6.

To a solution of tert-butyl (S)-3-(2-((((9H-fluoren-9-yl)methoxy)carbonyl)amino)-3-methoxy-3-oxopropyl)benzoate (18.00 g, 35.9 mmol) in THE (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THF. In the basic medium the mixture was extracted with diethyl ether to remove the non polar impurities. The aqueous layer was acidified with aqueous citric acid solution and extracted with ethyl acetate. The organic layer was dried over sodium sulphate and concentrated under reduced to get the desired compound as a gummy solid which was further lyophilized to give (S)-2-((((9H-fluoren-9-yl)methoxy)carbonyl)amino)-3-(3-(tert-butoxycarbonyl)phenyl)propanoic acid (16 g, 32.72 mmol, quantitive yield) as off-white solids. 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl)methoxy)carbonyl)amino)-3-(m-tolyl)propanoic acid

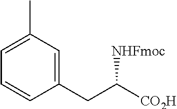

Compound was synthesized following the similar procedures of (S)-2-((((9H-fluoren-9-yl)methoxy)carbonyl)amino)-3-(3-(tert-butoxycarbonyl)phenyl)propanoic acid. Analysis condition E: Retention time=3.147 min; ESI-MS(+) m/z [M+H]⁺: 402.0. ¹H NMR (DMSO-d₆, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

### Preparation ethyl (S)-5-((tert-butoxycarbonyl)amino)-2-(((S)-mesitylsulfinyl)amino)-3, 3-dimethylpentanoate

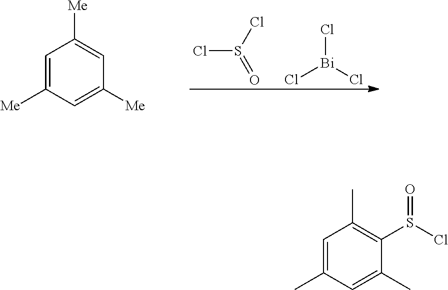

Step 1: The compound was synthesized using similar procedure described in reference: To a 1000-ml flask equipped with a septum inlet and magnetic stirring bar was added bismuth(III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding to an orange liquid. In order to remove the catalyst, 2000 mL of pentane were added, stirred and filtered through celite, and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

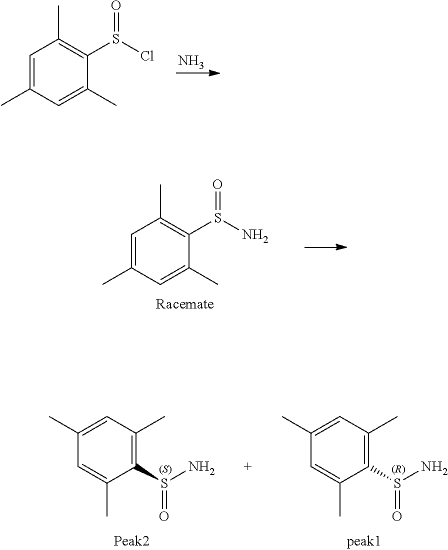

Step 2. The compound was synthesized using similar procedure described in reference: To a stirred solution of 2,4,6-trimethylbenzenesulfinic chloride (155 g, 765 mmol) in diethyl ether (1500 mL). After it had been cooled to −40° C. In a separate setup, (2 L multi neck RBF) taken in diethyl ether (900 mL) ammonia gas was bubbled 30 minutes at −40° C., this purged solution was added to above reaction mass at −40° C. After it had warmed to rt the reaction mixture was stirred for 2 hours and monitored by open access LCMS starting material was absent. The reaction was stirred at room temperature overnight according to given procedure. The reaction was monitored by TLC and open access LCMS, TLC wise starting material was absent. Workup: The reaction mixture was diluted with ethyl acetate (3000 mL) and washed with water(2000 ml), the organic layer was separated and the aqueous phase was again extracted with ethyl acetate(1×500 mL).The combined organic layer washed with brine(1×800 mL). The combined organic layer, dried (Na₂SO₄), filtered, and concentrated under reduced pressure to obtained (235 g) as a pale brown solid. The product (235 g) was recrystallized from 10% ethyl acetate/petroleum ether (500 mL), stirred, filtered, and dried to afford mesitylenesulphinamid (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d₆) δ 7.01-6.68 (m, 2H), 6.23-5.77 (m, 2H), 2.52-2.50 (m, 6H), 2.32-1.93 (m, 3H) and Peak-2 (desired): (S)-2, 4,6-trimethylbenzenesulfinamide (51.6 g, 267 mmol, 35.0% yield) as a white colour solid. ¹H NMR (400 MHz, DMSO-d₆) δ 6.87 (s, 2H), 6.16-5.82 (m, 2H), 2.53-2.50 (m, 6H), 2.34-1.93 (m, 3H).

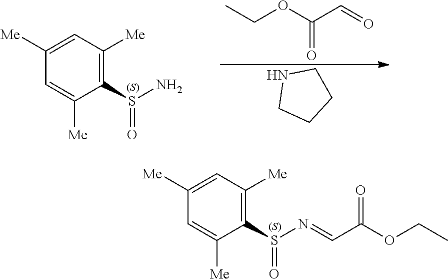

Step 3. The compound was synthesized using similar procedure described in reference: To a well stirred solution of (S)-2,4,6-trimethylbenzenesulfinamide (15.5 g, 85 mmol) in dichloromethane (235 mL) and 4 A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction was mass was filtered throw the celite and the bed was washed with DCM. The solvents were removed under reduced pressure to obtained the crude (55 g) as a brownish color mass. The crude compound was purified by ISCO (Column size: 300 g silica column. Adsorbent: 60-120 silica mesh, Mobile phase:40% EtOAc/Pet ether) and the product was collected at 15-20% of EtOAc. The fractions were concentrated to obtain ethyl (S,E)-2-((mesitylsulfinyl)imino)acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

Step 4. General procedure for the synthesis of TCNHPI redox-active esters as in reference ACIE: TCNHPI esters were prepared according to the previously reported general procedure (ACIE paper and references therein): A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of Celite®, SiO₂, or frit funnel) and insed with additional CH₂Cl₂/Et₂O. The solvent was removed under reduced pressure, and purification of the crude mixture by column chromatography afforded the desired TCNHPI redox-active ester. If necessary, the TCNHPI redox-active ester could be further recrystallized from CH₂Cl₂/MeOH.

Step 5. 4,5,6, 7-tetrachloro-1, 3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et₂O) afforded 2.15 g (84%) of the title compound. ¹H NMR (400 MHz, CDCl₃): δ 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂₀Cl₄N₂NaO₆ [M+Na]⁺: 534.9968, found: 534.9973.

Step 6. Ethyl (S)-5-((tert-butoxycarbonyl)amino)-2-(((S)-mesitylsulfinyl)amino)-3, 3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)₂·4H₂O (0.25 mmol, 25 mol %), Zinc (3 mmol, 3 equiv). The tube was then evacuated and backfilled with argon (three times). Anhydrous NMP (5.0 mL, 0.2 M) was added using a syringe. The mixture was stirred overnight at rt. Then, the reaction mixture was diluted with EtOAc, washed with water, brine and dried over MgSO₄. Upon filtration, the organic layer was concentrated under reduced pressure (water bath at 30° C.), and purified by flash column chromatography (silica gel) to provide the product. Purification by column (2:1 hexanes:EtOAc) afforded 327.6 mg (72%) of the title compound ethyl (S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5, 61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

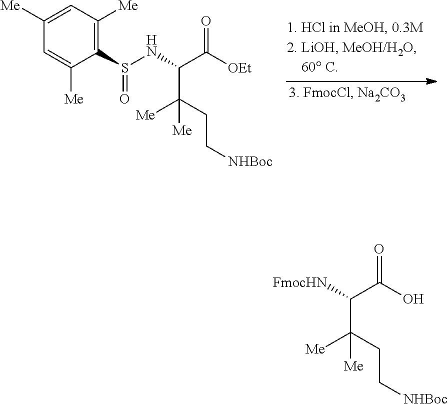

Step 7. 2-((((9H-fluoren-9-yl)methoxy)carbonyl)amino)-5-((tert-butoxycarbonyl)amino)-3,3-dimethylpentanoic acid: A culture tube was charged with ethyl (S)-5-((tert-butoxycarbonyl)amino)-2-(((S)-mesitylsulfinyl)amino)-3,3-dimethylpentanoate (0.5 mmol, 1.0 equiv), HCl (4.0 equiv) in MeOH (0.3 M) was added via syringe and the resulting mixture was stirred at RT for ca. 10 min (screened by TLC). After the reaction, Et₃N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to rt. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes EtOAc) to afford the product ethyl (S)-5-((tert-butoxycarbonyl)amino)-2-(((S)-mesitylsulfinyl)amino)-3,3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of (S)-2-((((9H-fluoren-9-yl)methoxy)carbonyl)amino)-3-(4,4-difluorocyclohexyl)propanoic acid

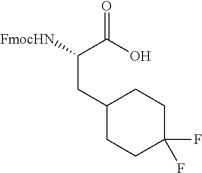

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired (S)-2-((((9H-fluoren-9-yl)methoxy)carbonyl)amino)-3-(4,4-difluorocyclohexyl)propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)-5-(tert-butoxy)-2-({[(9H-fluoren-9-yl)methoxy]carbonyl}amino)-3, 3-dimethyl-5-oxopentanoic acid

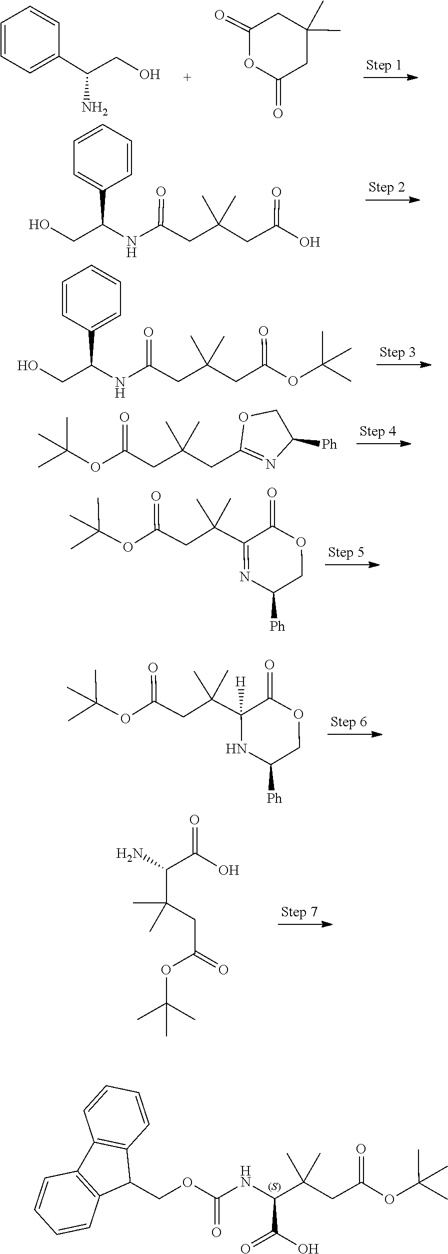

### Step 1

A solution of 4,4-dimethyldihydro-2H-pyran-2,6(3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d₆) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

### Step 2

(R)-5-((2-Hydroxy-1-phenylethyl)amino)-3,3-dimethyl-5-oxopentanoic acid (12 g, 43.0 mmol) was dissolved in a solution of benzyltrimethylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂/MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LC/MS Condition M: 1.96 min, 336.3 [M+H]⁺. ¹H NMR (300 MHz, DMSO-d₆) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

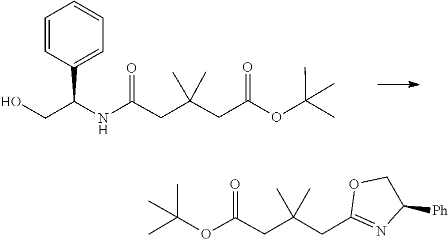

### Step 3

tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1:5) to give tert-butyl (R)-3,3-dimethyl-4-(4-phenyl-4,5-dihydrooxazol-2-yl)butanoate (5.6 g, 17.64 mmol, 99% yield). ESI-MS(+) m/z: 318.3 [M+H]⁺. ¹H NMR (300 MHz, DMSO-d6) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

### Step 4

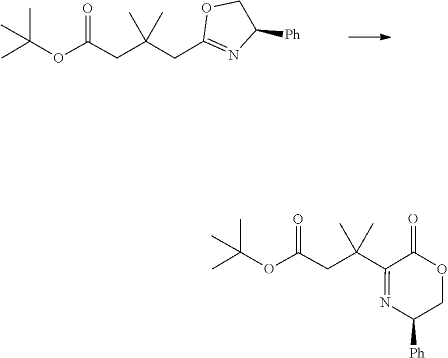 [SMILES: CC(C)(CC(=O)OC(C)(C)C)CC1=N[C@H](c2ccccc2)CO1 ; CC(C)(C)OC(=O)CC(C)(C)C1=N[C@H](c2ccccc2)COC1=O]

A solution of tert-butyl (R)-3,3-dimethyl-4-(4-phenyl-4,5-dihydrooxazol-2-yl)butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl)butanoate (1.3 g, 3.92 mmol, 22.23% yield) as a colorless liquid. ESI-MS(+) m/z: 332.2 [M+H]⁺. ¹H NMR (CDCl3) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). ¹³C NMR (CD3Cl) 6 26.40, 27.29, 28.00, 40.84, 45.94, 59.72, 70.88, 80.63, 127.13, 127.92, 128.65, 137.58, 155.07, 167.46, 171.95.

### Step 5

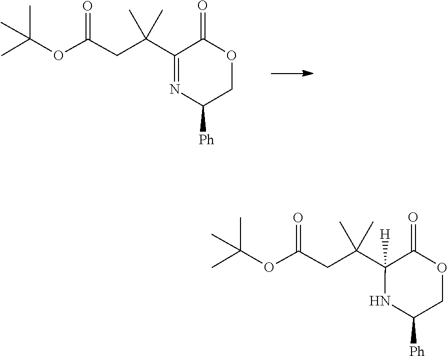 [SMILES: CC(C)(C)OC(=O)CC(C)(C)C1=N[C@H](c2ccccc2)COC1=O ; CC(C)(C)OC(=O)CC(C)(C)[C@@H]1N[C@H](c2ccccc2)COC1=O]

Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl)butanoate (1.3 g, 3.92 mmol) in methanol (50 mL). The reaction flask was purged with H₂ (3×) and stirred under H₂ for 24 h. After venting the vessel, the reaction mixture was filtered through Celite, and the filtrate was washed with EtOAc. The crude product was concentrated under vacuo and purified by flash column chromatography on silica gel (EtOAc/Hexanes, 1:8) to give tert-butyl 3-methyl-3-((3 S,5R)-2-oxo-5-phenylmorpholin-3-yl)butanoate (1.2 g, 3.33 mmol, 85% yield). ¹H NMR (300 MHz, DMSO-d₆) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

### Step 6.

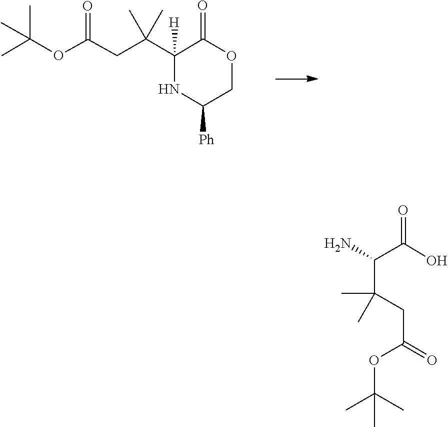 [SMILES: CC(C)(C)OC(=O)CC(C)(C)[C@@H]1N[C@H](c2ccccc2)COC1=O ; CC(C)(C)OC(=O)CC(C)(C)[C@H](N)C(=O)O]

Pearlman's catalyst Pd(OH)₂ on carbon (1.264 g, 1.799 mmol, 20% w w) was added to a solution of tert-butyl 3-methyl-3-((3S,5R)-2-oxo-5-phenylmorpholin-3-yl)butanoate (1.2 g, 3.60 mmol) in methanol (50 mL)/water (3.13 mL)/TFA (0.625 mL) (40:2.5:0.5, v/v/v). The vessel was purged with H₂ and stirred under H₂ for 24 h. After venting the vessel, the reaction mixture was filtered through Celite, and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

### Step 7.

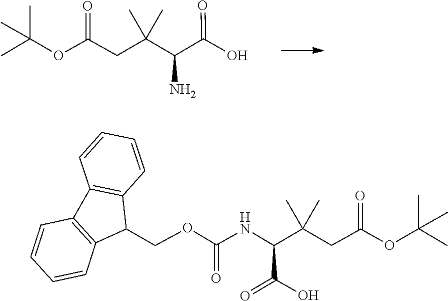 [SMILES: CC(C)(C)OC(=O)CC(C)(C)[C@H](N)C(=O)O ; CC(C)(C)OC(=O)CC(C)(C)[C@H](NC(=O)OCC1c2ccccc2-c2ccccc21)C(=O)O]

The crude product (S)-2-amino-5-(tert-butoxy)-3,3-dimethyl-5-oxopentanoic acid (1 g, 4.32 mmol) dissolved in water (30 mL). Na₂CO₃ (0.916 g, 8.65 mmol) was then added to the above solution. To this solution, fmoc n-hydroxysuccinimide ester (1.458 g, 4.32 mmol) in dioxane (30 mL) was added drop wise at 0° C. and stirred at room temperature for 16 h. The reaction mixture was acidified to pH˜2 by 1N HCl and extracted with EtOAc (50 mL×3), dried over Na₂SO₄, concentrated under vacuo and purified by flash column chromatography on silica gel (EtOAc/petroleum ether, 35 to 39%) to give (S)-2-((((9H-fluoren-9-yl)methoxy)carbonyl)amino)-5-(tert-butoxy)-3,3-dimethyl-5-oxopentanoic acid (0.73 g, 1.567 mmol, 36.2% yield) as a white solid. LCMS, Analytical LC/MS Condition E, MS (ESI) t_{R}=2.135 min, m/z 452.2 [M−H]⁻. ¹H NMR (400 MHz, DMSO-d₆) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl)methoxy]carbonyl}amino)-3-(morpholin-4-yl)propanoic acid

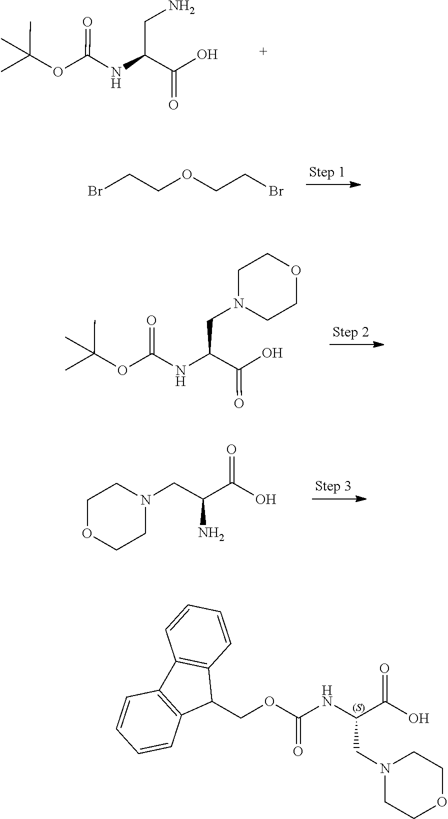

### Step 1

In a 2-L multi-necked round-bottomed flask fitted with a thermo pocket was added (S)-3-amino-2-((tert-butoxycarbonyl)amino)propanoic acid (50 g, 245 mmol), dioxane (500 mL), followed by 1-bromo-2-(2-bromoethoxy)ethane (30.8 mL, 245 mmol) at rt. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to get light red solution which was acidified to pH 3. The resulting mixture was concentrated under high vacuum pump (˜4 mbar) at 60° C. to get (S)-2-((tert-butoxycarbonyl)amino)-3-morpholinopropanoic acid (67 g, 244 mmol, 100% yield) pale yellow solid. Analytical LC/MS Condition M: 0.56 min, 275.2 [M+H]⁺.

### Step 2

To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at rt for 12 h. The volatile was evaporated to get pale yellow sticky crude (S)-2-amino-3-morpholinopropanoic acid (16 g), which was taken for next step without further purification. MS (ESI) m/z 175.2 [M+H]⁺.

### Step 3

The crude product (S)-2-amino-3-morpholinopropanoic acid (11 g, 63.1 mmol) was dissolved in water (250 mL. Na₂CO₃ (13.39 g, 126 mmol was then added to the above solution. To this solution, Fmoc-N-hydroxysuccinimide ester (21.30 g, 63.1 mmol was added dropwise at 0° C. and stirred at room temperature for 16 h. The reaction mixture was acidified to pH˜2 by 1N HCl and extracted with EtOAc (500 mL×3), dried over Na₂SO₄, concentrated under vacuo, and purified by flash column chromatography on silica gel (petroleum ether/EtOAc, 0-100% then MeOH/CHCl₃ 0-15%) to get (S)-2-((((9H-fluoren-9-yl)methoxy)carbonyl)amino)-3-morpholinopropanoic acid (23 g, 55.9 mmol, 89% yield) as a brown solid. Analytical LC/MS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy)carbonyl]amino}-2-({[(9H-fluoren-9-yl)methoxy]carbonyl}amino)butanoic acid

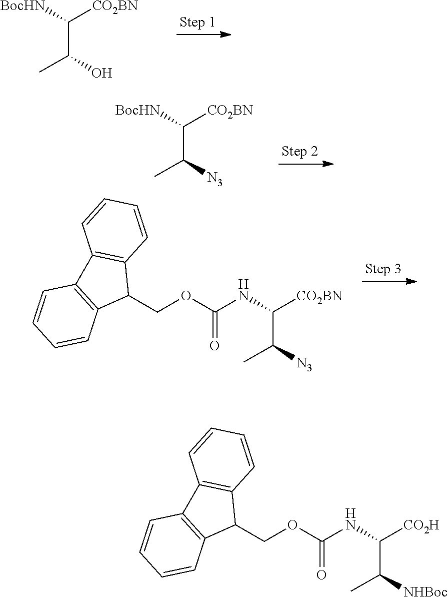

### Step 1

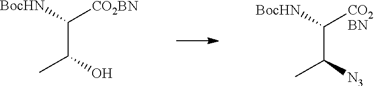

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex:EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex:EtOAc 95:5 a 9:1) to give benzyl (2S,3S)-3-azido-2-((tert-butoxycarbonyl)amino)butanoate (20 g, 59.8 mmol, 84% yield) as colorless liquid. Analytical LC/MS Condition E: 3.13 min, 333.2 [M−H]⁻.

### Step 2

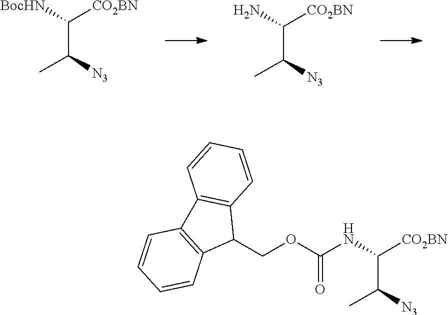

A solution of benzyl (2S,3S)-3-azido-2-((tert-butoxycarbonyl)amino)butanoate (20 g, 59.8 mmol), dichloromethane (300 mL) and TFA (50 mL, 649 mmol) was stirred for 2 h at 23° C. and then evaporated to dryness to give the corresponding amine. The above amine was redissolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to rt and stirred for 3 h. It was extracted with EtOAc and washed with 0.5 M HCl solution and then brine solution. It was concentrated to get crude liquid. The above crude was purifier by silica gel column chromatography. The product was eluted at 20% EtOAc in petroleum ether. The fractions were concentrated to get benzyl (2S,3S)-2-((((9H-fluoren-9-yl)methoxy)carbonyl)amino)-3-azidobutanoate (23 g, 50.4 mmol, 84% yield) as a colorless liquid. Analytical LC/MS Condition E: 3.70 min, 479.3 [M+Na]⁺.

### Step 3.

To a multi-neck round-bottled flask was charged benzyl (2S,3S)-2-((((9H-fluoren-9-yl)methoxy)carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirring under nitrogen for 12 h. The reaction mass was filtered through celite bed, washed the bed with THF/Water mixture. The mother liquid was concentrated and washed with EtOAc. Then pH of water layer was adjusted to 7-6 using 1.5 N HCl solution. The resulting white solid was extracted with ethylacetate. The above reaction was repeated three more times. The combined organics were washed with water and brine solution, dried over sodium sulphate, and concentrated to afford (2S,3S)-2-((((9H-fluoren-9-yl)methoxy)carbonyl)amino)-3-((tert-butoxycarbonyl)amino)butanoic acid as a white solid (28 g). This was mixed with a previously obtained batch (8 g) in DCM (200 mL). n-Hexane (1 L) was added to the above solution and sonicated for 2 min. The solids were filtered, rinsed with hexanes and dried overnight to give (2S,3S)-2-((((9H-fluoren-9-yl)methoxy)carbonyl)amino)-3-((tert-butoxycarbonyl)amino)butanoic acid (36 g, 81 mmol, 92% yield) as a white powder. Analytical LC/MS Condition E: 1.90 min, 439.2 [M−H]-. ¹H NMR (400 MHz, DMSO-d₆) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). 13C NMR (101 Hz, DMSO-d₆) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of (S)-2-((((9H-fluoren-9-yl)methoxy)carbonyl)amino)-2-(1-(((tert-butoxycarbonyl)amino)methyl)cyclopropyl)acetic acid

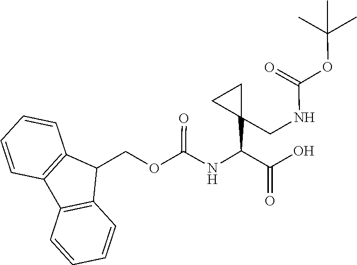 [SMILES: CC(C)(C)OC(=O)NCC1([C@H](NC(=O)OCC2c3ccccc3-c3ccccc32)C(=O)O)CC1]

The compound was obtained following similar procedures of ethyl (S)-5-((tert-butoxycarbonyl)amino)-2-(((S)-mesitylsulfinyl)amino)-3,3-dimethylpentanoate. The synthesis afforded the desired product (0.65 g, 22% yield) as a white solid after purification by flash column chromatography (Red Sep, 40 g, SiO2, 35 to 40% EtOAc:hexanes (compound ELSD active)). Analytical LC/MS Condition E: 2.04 min, 465.2 [M−H]⁻. ¹H NMR (300 MHz, DMSO-d₆) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of (S)-2-((((9H-fluoren-9-yl)methoxy)carbonyl)amino)-2-(1-(tert-butoxycarbonyl)azetidin-3-yl)acetic acid

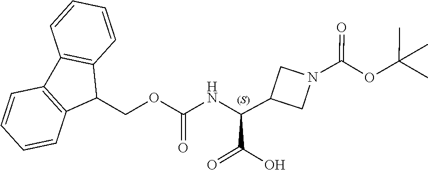

The compound was obtained following similar procedures of ethyl (S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E: 1.87 min, 467.2 [M−H]⁻. ¹H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

### Example 2: Preparation of Compounds of Formula (I)

### Preparation of Compound 1000

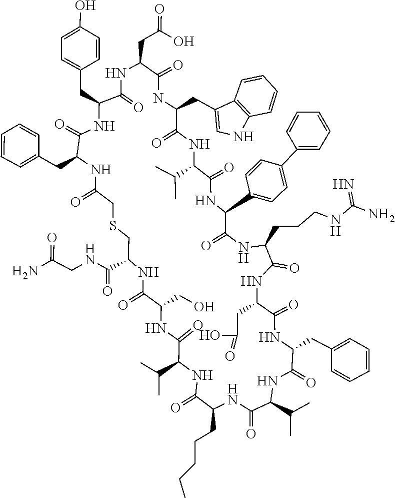

To a 45-mL polypropylene solid-phase reaction vessel was added using Siebber or Rink resin on a 50 μmol scale, and the reaction vessel was placed on the Symphony peptide synthesizer. The following procedures were then performed sequentially: “Symphony Resin-swelling procedure” was followed; “Symphony Single-coupling procedure” was followed with Fmoc-Gly-OH; “Symphony Single-coupling procedure” was followed with Fmoc-Cys(Trt)-OH; “Symphony Single-coupling procedure” was followed with Fmoc-Ser(tBu)-OH; “Symphony Single-coupling procedure” was followed with Fmoc-Val-OH; “Symphony Single-coupling procedure” was followed with Fmoc-Ahp-OH; “Symphony Single-coupling procedure” was followed with Fmoc-Val-OH; “Symphony Single-coupling procedure” was followed with Fmoc-D-Phe-OH; “Symphony Single-coupling procedure” was followed with Fmoc-Asp-OH; “Symphony Single-coupling procedure” was followed with Fmoc-Arg(Pbf)-OH; “Symphony double-coupling procedure” was followed with Fmoc-Bip-OH; “Symphony single-coupling procedure” was followed with Fmoc-Val-OH; “Symphony single-coupling procedure” was followed with Fmoc-Trp(Boc)-OH; “Symphony single-coupling procedure” was followed with Fmoc-Asp(tBu)-OH; “Symphony Single-coupling procedure” was followed with Fmoc-Tyr(tBu)-OH; “Symphony Single-coupling procedure” was followed with Fmoc-Phe-OH; “Symphony Chloroacetic Anhydride coupling procedure” was followed; “Global Deprotection Method A” was followed: “Cyclization Method” was followed.

The crude material was purified via preparative LC/MS with the following conditions: Column: waters xbridge C-18, 30×150 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 6.3 mg, and its estimated purity by LCMS analysis was 97%.

Analysis condition A: Retention time=1.72 min; ESI-MS(+) m/z [M+H]⁺: 1981.7.

Analysis condition B: Retention time=1.86 min; ESI-MS(+) m/z [M+H]⁺: 1981.9.

### Preparation of Compound 1001

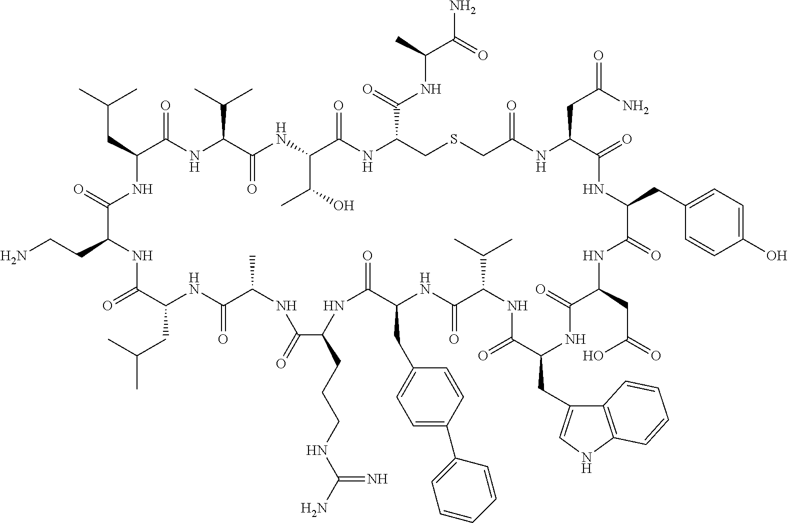 [SMILES: CC(C)C[C@@H]1NC(=O)[C@H](CCN)NC(=O)[C@@H](CC(C)C)NC(=O)[C@H](C)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

To a 45-mL polypropylene solid-phase reaction vessel was added using Siebber or Rink resin on a 50 μmol scale, and the reaction vessel was placed on the Symphony X peptide synthesizer. The following procedures were then performed sequentially: “Symphony X Resin-swelling procedure” was followed; “Symphony X Single-coupling procedure” was followed with Fmoc-Ala-OH; “Symphony X Single-coupling procedure” was followed with Fmoc-Cys(Trt)-OH; “Symphony X Single-coupling procedure” was followed with Fmoc-Thrr(tBu)-OH; “Symphony X Single-coupling procedure” was followed with Fmoc-Val-OH; “Symphony X Single-coupling procedure” was followed with Fmoc-Leu-OH; “Symphony X Single-coupling procedure” was followed with Fmoc-Dab(Boc)-OH; “Symphony X Single-coupling procedure” was followed with Fmoc-D-Leu-OH; “Symphony X Single-coupling procedure” was followed with Fmoc-Ala-OH; “Symphony X Single-coupling procedure” or “Symphony X double-coupling procedure” was followed with Fmoc-Arg(Pbf)-OH; “Symphony X single-coupling procedure” was followed with Fmoc-Bip-OH; “Symphony X single-coupling procedure” was followed with Fmoc-Val-OH; “Symphony X single-coupling procedure” was followed with Fmoc-Trp(Boc)-OH; “Symphony X single-coupling procedure” was followed with Fmoc-Asp(tBu)-OH; “Symphony X Single-coupling procedure” was followed with Fmoc-Tyr(tBu)-OH; “Symphony X Single-coupling procedure” was followed with Fmoc-Asn(Trt)-OH; “Symphony X Chloroacetic Anhydride coupling procedure” was followed; “Global Deprotection Method A” was followed; “Cyclization Method” was followed.

The crude material was purified via preparative LC/MS with the following conditions: Column: XBridge C18, 30×200 mm, 5-μm particles; Mobile Phase A: 5:95 acetonitrile: water with 10-mM ammonium acetate; Mobile Phase B: 95:5 acetonitrile: water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 2.9 mg, and its estimated purity by LCMS analysis was 92%.

Analysis condition A: Retention time=1.66 min; ESI-MS(+) m/z [M+H]⁺: 1886.2.

Analysis condition B: Retention time=1.57 min; ESI-MS(+) m/z [M+2H]²⁺: 944.1.

### Preparation of Compound 1002

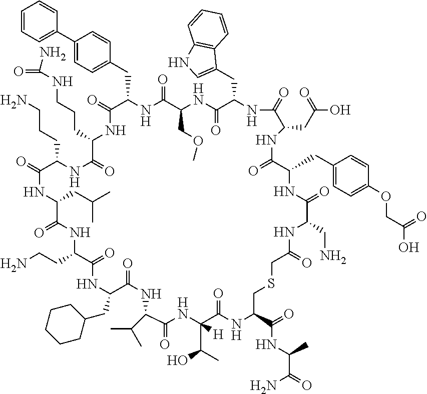 [SMILES: COC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

To a 45-mL polypropylene solid-phase reaction vessel was added Rink resin (470 mg, 0.25 mmol), and the reaction vessel was placed on the Prelude peptide synthesizer. The following procedures were then performed sequentially: “Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Thr(tBu)-OH; “Prelude Single-coupling procedure” was followed with Fmoc-Val-OH; “Prelude Single-coupling procedure” was followed with Fmoc-Cha-OH; “Prelude Single-coupling procedure” was followed with Fmoc-Dab(Boc)-OH; “Prelude Single-coupling procedure” was followed with Fmoc-D-Leu-OH; “Prelude Single-coupling procedure” was followed with Fmoc-Orn(Boc)-OH; “Prelude Single-coupling procedure” was followed with Fmoc-Cit-OH; “Prelude Single-coupling procedure” was followed with Fmoc-Bip-OH; The resin was split into 0.050 mmol and was transferred to a 45-mL polypropylene solid-phase reaction vessel, and it was placed on the Symphony peptide synthesizer. The following procedures were then performed sequentially: “Symphony Single-coupling procedure” was followed with Fmoc-Ser(Me)-OH; “Symphony Single-coupling procedure” was followed with Fmoc-Trp(Boc)-OH; “Symphony Single-coupling procedure” was followed with Fmoc-Asp(tBu)-OH; “Symphony Single-coupling procedure” was followed with Tyr(CH₂COOtBu)-OH; “Symphony Single-coupling procedure” was followed with Fmoc-Dap(Boc)-OH; “Symphony Chloroacetic Anhydride coupling procedure” was followed; “Symphony Final rinse and dry procedure” was followed; “Global Deprotection Method A” was followed; “Cyclization Method A” was followed.

The crude material was purified via preparative LC/MS with the following conditions: Column: XBridge C18, 200 mm×19 mm, 5-μm particles; Mobile Phase A: 5:95 acetonitrile: water with 10-mM ammonium acetate; Mobile Phase B: 95:5 acetonitrile: water with 10-mM ammonium acetate; Gradient: a 0-minute hold at 16% B, 16-56% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The material was further purified via preparative LC/MS with the following conditions: Column: XBridge C18, 200 mm×19 mm, 5-μm particles; Mobile Phase A: 5:95 acetonitrile: water with 0.1% trifluoroacetic acid; Mobile Phase B: 95:5 acetonitrile: water with 0.1% trifluoroacetic acid; Gradient: a 0-minute hold at 33% B, 33-55% B over 25 minutes, then a 2-minute hold at 100% B; Flow Rate: 20 mL/min; Column Temperature: 25 C. Fraction collection was triggered by UV signals. Fractions containing the desired product were combined and dried via centrifugal evaporation. The yield of the product was 15.1 mg, and its estimated purity by LCMS analysis was 96%.

Analysis condition A: Retention time=1.43 min; ESI-MS(+) m/z [M+2H]²⁺: 1002.2.

Analysis condition B: Retention time=1.42 min; ESI-MS(+) m/z [M+2H]²⁺: 1002.0.

Following methods described in general synthetic and purification procedures and in Compounds 1000-1002 and 2048, Compounds 1003-1865, 2000-2330, and 2500-2707 were obtained.

### Preparation of Compound 1003

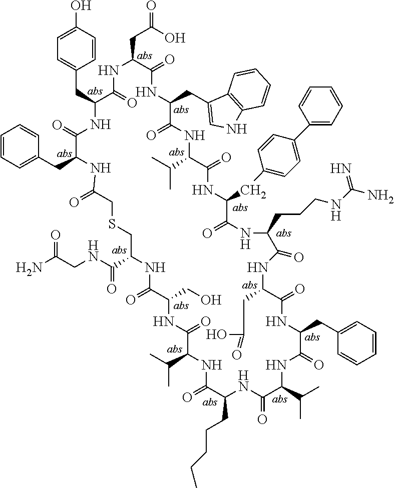 [SMILES: CCCCC[C@@H]1NC(=O)[C@H](C(C)C)NC(=O)[C@H](Cc2ccccc2)NC(=O)[C@H](CC(=O)O)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Compound 1003 was prepared on a 50 μmol scale. The yield of the product was 2.4 mg, and its estimated purity by LCMS analysis was 100%. Analysis condition A: Retention time=1.84 min; ESI-MS(+) m/z [M+2H]²⁺: 992.

### Preparation of Compound 1004

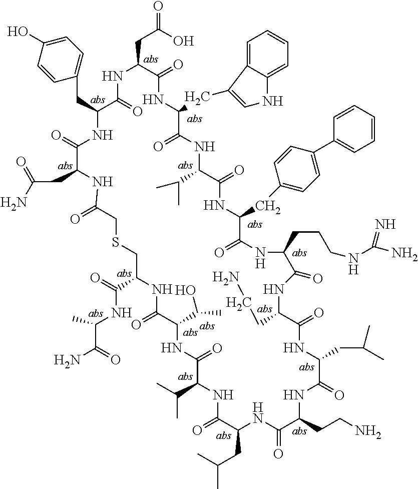 [SMILES: CC(C)C[C@@H]1NC(=O)[C@H](CCN)NC(=O)[C@@H](CC(C)C)NC(=O)[C@H](CCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1004 was prepared on a 50 μmol scale. The yield of the product was 7 mg, and its estimated purity by LCMS analysis was 96.8%. Analysis condition A: Retention time=1.62 min; ESI-MS(+) m/z [M+2H]²⁺: 1915.2.

### Preparation of Compound 1005

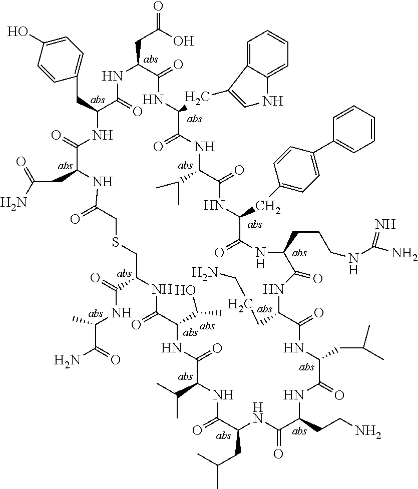 [SMILES: CC(C)C[C@@H]1NC(=O)[C@H](CCN)NC(=O)[C@@H](CC(C)C)NC(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1005 was prepared on a 50 μmol scale. The yield of the product was 10.9 mg, and its estimated purity by LCMS analysis was 95.3%. Analysis condition B: Retention time=1.39 min; ESI-MS(+) m/z [M+3H]³⁺: 644.1.

### Preparation of Compound 1006

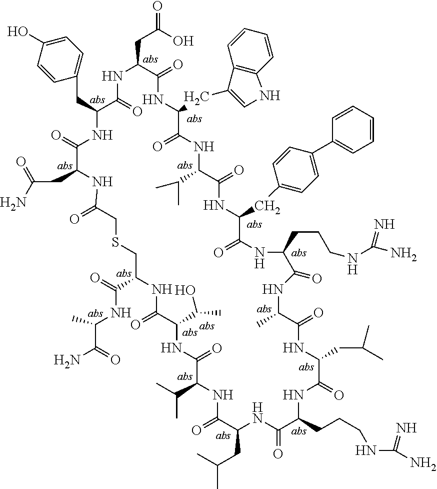 [SMILES: CC(C)C[C@@H]1NC(=O)[C@H](CCCNC(=N)N)NC(=O)[C@@H](CC(C)C)NC(=O)[C@H](C)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1006 was prepared on a 50 μmol scale. The yield of the product was 18.5 mg, and its estimated purity by LCMS analysis was 98.5%. Analysis condition B: Retention time=1.44 min; ESI-MS(+) m/z [M+2H]²⁺: 972.1.

### Preparation of Compound 1007

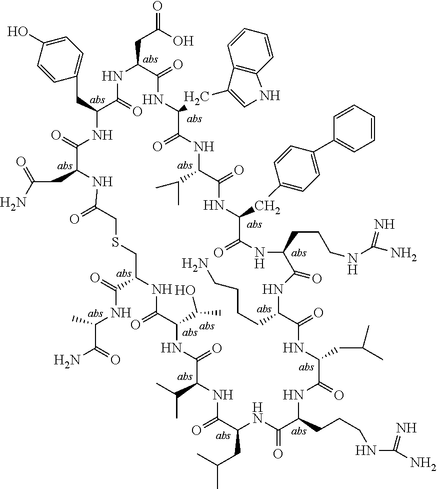 [SMILES: CC(C)C[C@@H]1NC(=O)[C@H](CCCNC(=N)N)NC(=O)[C@@H](CC(C)C)NC(=O)[C@H](CCCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1007 was prepared on a 50 mol scale. The yield of the product was 41 mg, and its estimated purity by LCMS analysis was 96.1%. Analysis condition B: Retention time=1.28, 1.31 min; ESI-MS(+) m/z [M+2H]²⁺: 1001.

### Preparation of Compound 1008

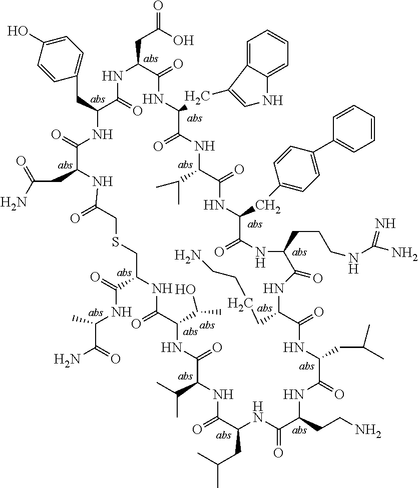 [SMILES: CC(C)C[C@@H]1NC(=O)[C@H](CCN)NC(=O)[C@@H](CC(C)C)NC(=O)[C@H](CCCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1008 was prepared on a 50 mol scale. The yield of the product was 32.3 mg, and its estimated purity by LCMS analysis was 96.6%. Analysis condition A: Retention time=1.33 min; ESI-MS(+) m/z [M+3H]³⁺: 648.8.

### Preparation of Compound 1009

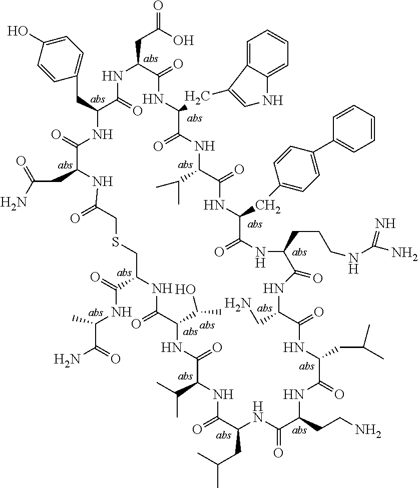 [SMILES: CC(C)C[C@@H]1NC(=O)[C@H](CCN)NC(=O)[C@@H](CC(C)C)NC(=O)[C@H](CN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1009 was prepared on a 50 μmol scale. The yield of the product was 30.4 mg, and its estimated purity by LCMS analysis was 95.8%. Analysis condition B: Retention time=1.26 min; ESI-MS(+) m/z [M+3H]³⁺: 634.9.

### Preparation of Compound 1010

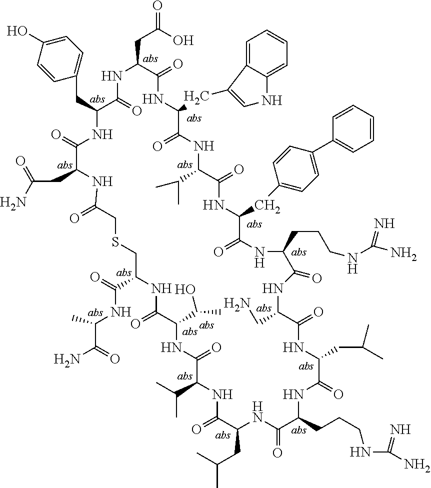 [SMILES: CC(C)C[C@@H]1NC(=O)[C@H](CCCNC(=N)N)NC(=O)[C@@H](CC(C)C)NC(=O)[C@H](CN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1010 was prepared on a 50 μmol scale. The yield of the product was 17 mg, and its estimated purity by LCMS analysis was 92.7%. Analysis condition B: Retention time=1.27 min; ESI-MS(+) m/z [M+3H]³⁺: 653.3.

### Preparation of Compound 1011

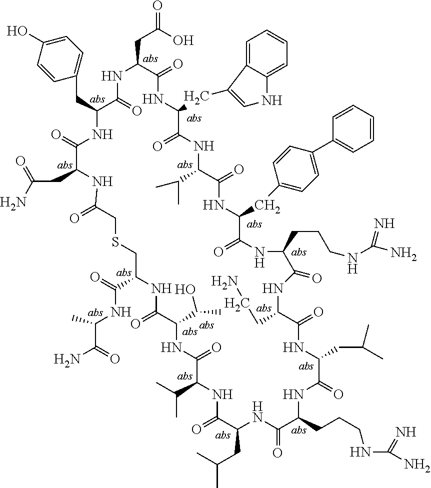 [SMILES: CC(C)C[C@@H]1NC(=O)[C@H](CCCNC(=N)N)NC(=O)[C@@H](CC(C)C)NC(=O)[C@H](CCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1011 was prepared on a 50 μmol scale. The yield of the product was 44.5 mg, and its estimated purity by LCMS analysis was 95.9%. Analysis condition B: Retention time=1.27 min; ESI-MS(+) m/z [M+3H]³⁺: 658.2.

### Preparation of Compound 1012

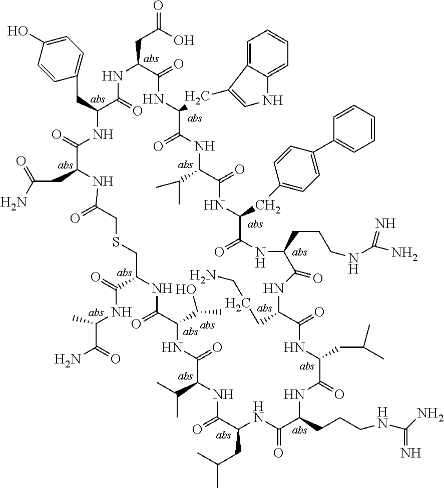 [SMILES: CC(C)C[C@@H]1NC(=O)[C@H](CCCNC(=N)N)NC(=O)[C@@H](CC(C)C)NC(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1012 was prepared on a 50 μmol scale. The yield of the product was 20.1 mg, and its estimated purity by LCMS analysis was 98.2%. Analysis condition B: Retention time=1.4 min; ESI-MS(+) m/z [M+H]⁺: 1985.2.

### Preparation of Compound 1013

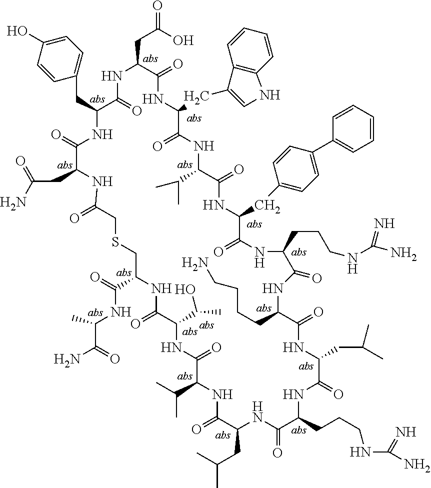 [SMILES: CC(C)C[C@@H]1NC(=O)[C@H](CCCNC(=N)N)NC(=O)[C@@H](CC(C)C)NC(=O)[C@@H](CCCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1013 was prepared on a 50 μmol scale. The yield of the product was 31.7 mg, and its estimated purity by LCMS analysis was 92.3%. Analysis condition B: Retention time=1.25 min; ESI-MS(+) m/z [M+2H]²⁺: 1000.8.

### Preparation of Compound 1014

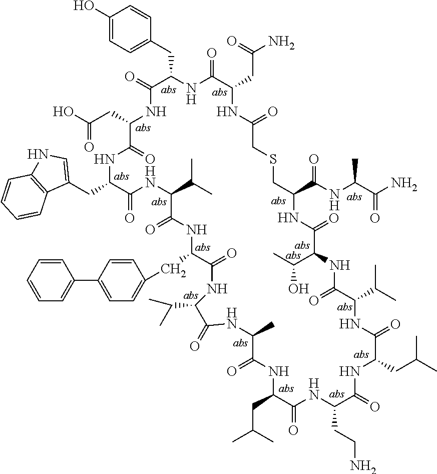 [SMILES: CC(C)C[C@@H]1NC(=O)[C@H](CCN)NC(=O)[C@@H](CC(C)C)NC(=O)[C@H](C)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1014 was prepared on a 50 μmol scale. The yield of the product was 3.9 mg, and its estimated purity by LCMS analysis was 98%. Analysis condition B: Retention time=1.55 min; ESI-MS(+) m/z [M+H]⁺: 1828.9.

### Preparation of Compound 1015

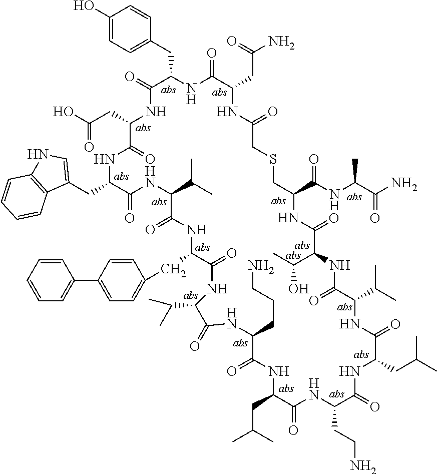 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1015 was prepared on a 50 μmol scale. The yield of the product was 23.4 mg, and its estimated purity by LCMS analysis was 93.2%. Analysis condition B: Retention time=1.36 min; ESI-MS(+) m/z [M+2H]²⁺: 937.

### Preparation of Compound 1016

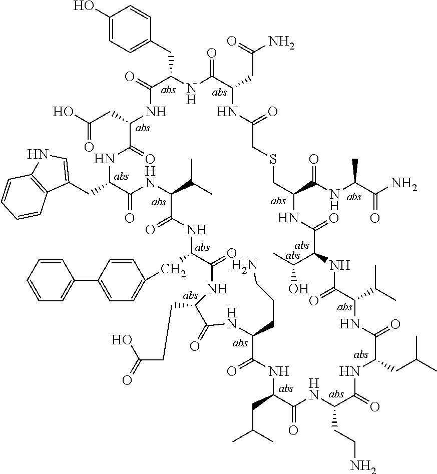 [SMILES: CC(C)C[C@@H]1NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1016 was prepared on a 50 μmol scale. The yield of the product was 9.5 mg, and its estimated purity by LCMS analysis was 93.8%. Analysis condition B: Retention time=1.34 min; ESI-MS(+) m/z [M+2H]²⁺: 952.2.

### Preparation of Compound 1017

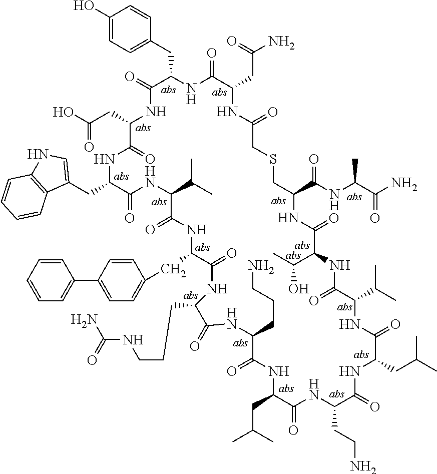 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1017 was prepared on a 50 μmol scale. The yield of the product was 13 mg, and its estimated purity by LCMS analysis was 92.2%. Analysis condition B: Retention time=1.33 min; ESI-MS(+) m/z [M+2H]²⁺: 966.

### Preparation of Compound 1018

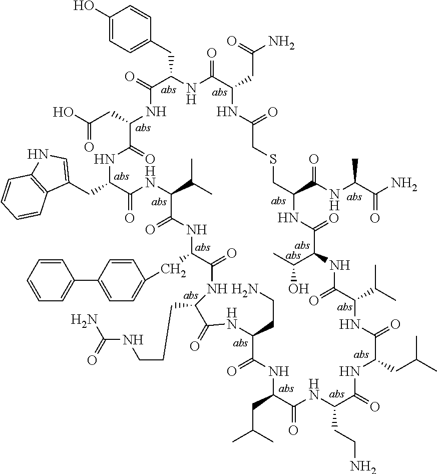 [SMILES: CC(C)C[C@@H]1NC(=O)[C@H](CCN)NC(=O)[C@@H](CC(C)C)NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1018 was prepared on a 50 μmol scale. The yield of the product was 18.2 mg, and its estimated purity by LCMS analysis was 90.8%. Analysis condition B: Retention time=1.33 min; ESI-MS(+) m/z [M+2H]²⁺: 959.2.

### Preparation of Compound 1019

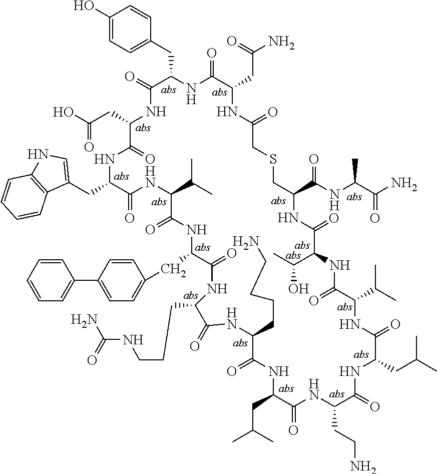 [SMILES: CC(C)C[C@@H]1NC(=O)[C@H](CCN)NC(=O)[C@@H](CC(C)C)NC(=O)[C@H](CCCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1019 was prepared on a 50 μmol scale. The yield of the product was 11.9 mg, and its estimated purity by LCMS analysis was 96%. Analysis condition A: Retention time=1.35 min; ESI-MS(+) m/z [M+2H]²⁺: 973.3.

### Preparation of Compound 1020

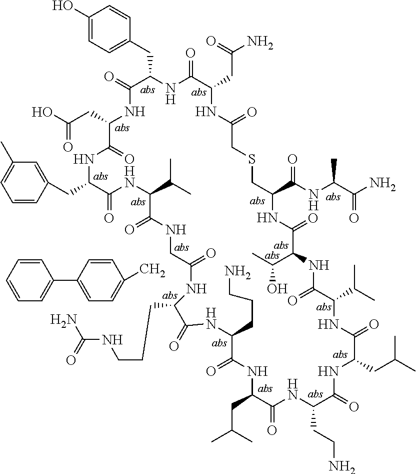

Compound 1020 was prepared on a 50 μmol scale. The yield of the product was 6 mg, and its estimated purity by LCMS analysis was 88.8%. Analysis condition B: Retention time=1.4 min; ESI-MS(+) m/z [M+2H]²⁺: 952.4.

### Preparation of Compound 1021

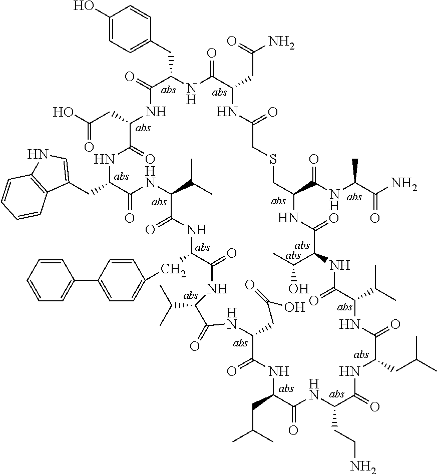 [SMILES: CC(C)C[C@@H]1NC(=O)[C@H](CCN)NC(=O)[C@@H](CC(C)C)NC(=O)[C@@H](CC(=O)O)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1021 was prepared on a 50 μmol scale. The yield of the product was 11.8 mg, and its estimated purity by LCMS analysis was 97.5%. Analysis condition A: Retention time=1.38 min; ESI-MS(+) m/z [M+2H]²⁺: 937.

### Preparation of Compound 1022

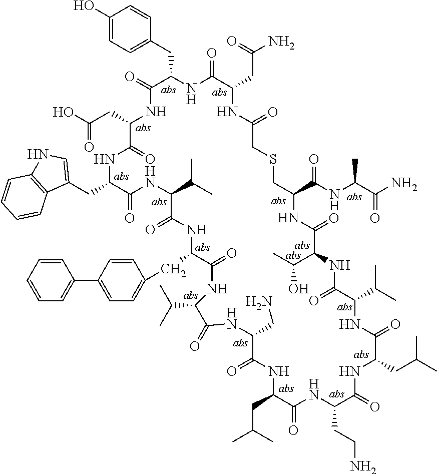 [SMILES: CC(C)C[C@@H]1NC(=O)[C@H](CCN)NC(=O)[C@@H](CC(C)C)NC(=O)[C@@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1022 was prepared on a 50 μmol scale. The yield of the product was 18.9 mg, and its estimated purity by LCMS analysis was 92%. Analysis condition B: Retention time=1.4 min; ESI-MS(+) m/z [M+2H]²⁺: 923.1.

### Preparation of Compound 1023

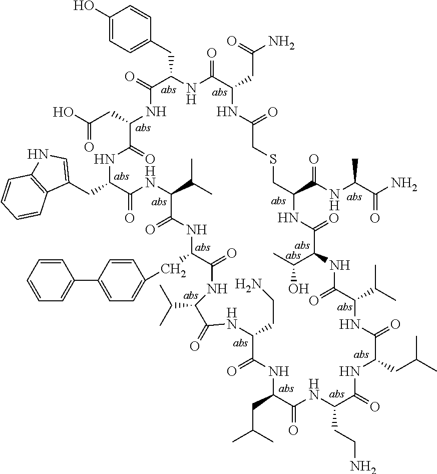 [SMILES: CC(C)C[C@@H]1NC(=O)[C@H](CCN)NC(=O)[C@@H](CC(C)C)NC(=O)[C@@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1023 was prepared on a 50 μmol scale. The yield of the product was 15.1 mg, and its estimated purity by LCMS analysis was 96.6%. Analysis condition A: Retention time=1.39 min; ESI-MS(+) m/z [M+2H]²⁺: 930.

### Preparation of Compound 1024

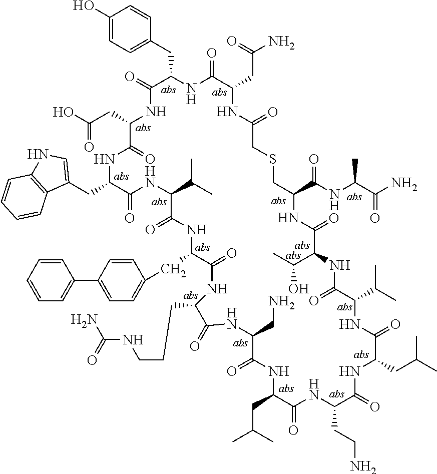 [SMILES: CC(C)C[C@@H]1NC(=O)[C@H](CCN)NC(=O)[C@@H](CC(C)C)NC(=O)[C@H](CN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1024 was prepared on a 50 μmol scale. The yield of the product was 22.2 mg, and its estimated purity by LCMS analysis was 93.2%. Analysis condition B: Retention time=1.34 min; ESI-MS(+) m/z [M+2H]²⁺: 951.2.

### Preparation of Compound 1025

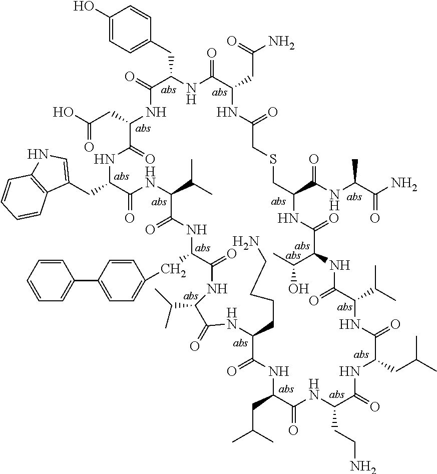 [SMILES: CC(C)C[C@@H]1NC(=O)[C@H](CCN)NC(=O)[C@@H](CC(C)C)NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1025 was prepared on a 50 μmol scale. The yield of the product was 5.5 mg, and its estimated purity by LCMS analysis was 96%. Analysis condition A: Retention time=1.45 min; ESI-MS(+) m/z [M+2H]²⁺: 944.1.

### Preparation of Compound 1026

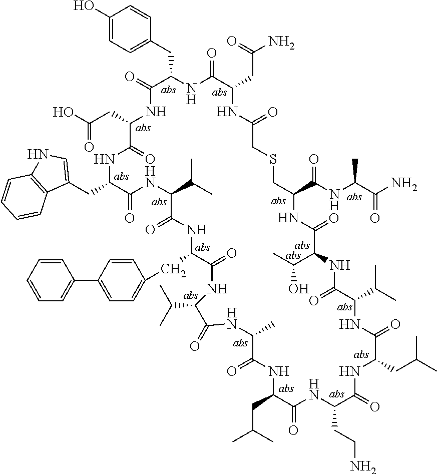 [SMILES: CC(C)C[C@@H]1NC(=O)[C@H](CCN)NC(=O)[C@@H](CC(C)C)NC(=O)[C@@H](C)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1026 was prepared on a 50 μmol scale. The yield of the product was 7.8 mg, and its estimated purity by LCMS analysis was 92.7%. Analysis condition A: Retention time=1.45 min; ESI-MS(+) m/z [M+2H]²⁺: 915.3.

### Preparation of Compound 1027

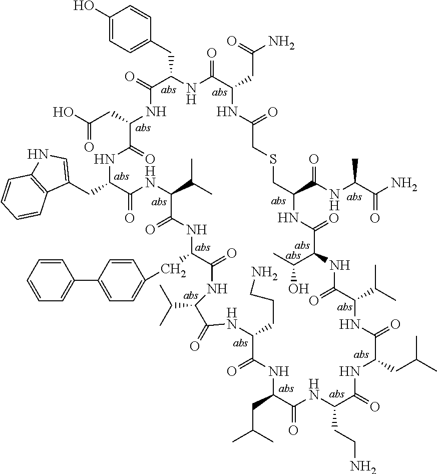 [SMILES: CC(C)C[C@@H]1NC(=O)[C@H](CCN)NC(=O)[C@@H](CC(C)C)NC(=O)[C@@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1027 was prepared on a 50 μmol scale. The yield of the product was 18.4 mg, and its estimated purity by LCMS analysis was 84.4%. Analysis condition B: Retention time=1.32 min; ESI-MS(+) m/z [M+2H]²⁺: 937.1.

### Preparation of Compound 1028

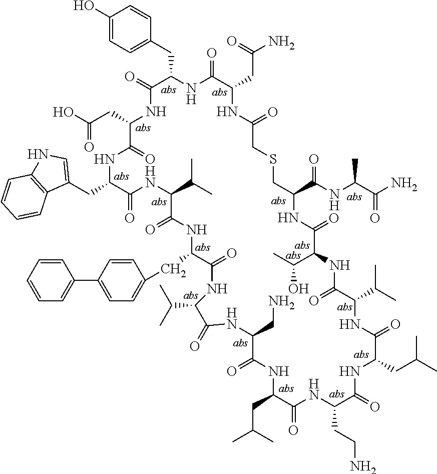 [SMILES: CC(C)C[C@@H]1NC(=O)[C@H](CCN)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1028 was prepared on a 50 μmol scale. The yield of the product was 7.8 mg, and its estimated purity by LCMS analysis was 90.7%. Analysis condition B: Retention time=1.36 min; ESI-MS(+) m/z [M+2H]²⁺: 923.2.

### Preparation of Compound 1029

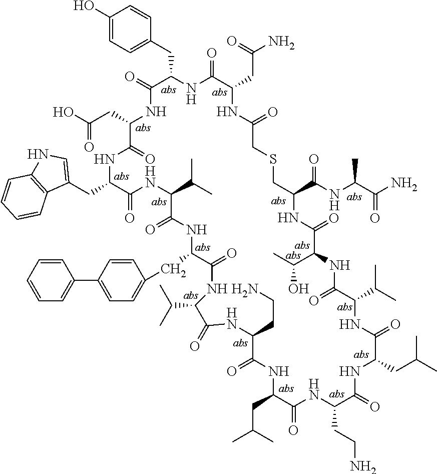 [SMILES: CC(C)C[C@@H]1NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1029 was prepared on a 50 mol scale. The yield of the product was 10.4 mg, and its estimated purity by LCMS analysis was 91%. Analysis condition B: Retention time=1.36 min; ESI-MS(+) m/z [M+2H]²⁺: 930.3.

### Preparation of Compound 1030

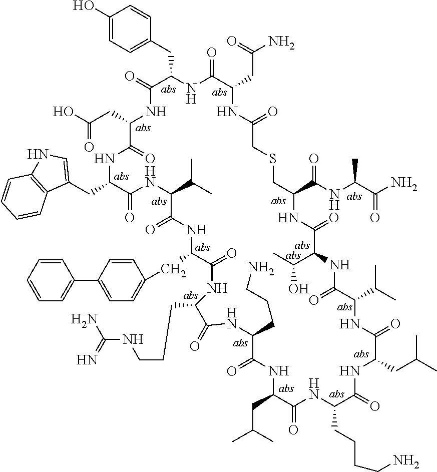 [SMILES: CC(C)C[C@@H]1NC(=O)[C@H](CCCCN)NC(=O)[C@@H](CC(C)C)NC(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1030 was prepared on a 50 μmol scale. The yield of the product was 12 mg, and its estimated purity by LCMS analysis was 95.4%. Analysis condition B: Retention time=1.55 min; ESI-MS(+) m/z [M+H]⁺: 1956.9.

### Preparation of Compound 1031

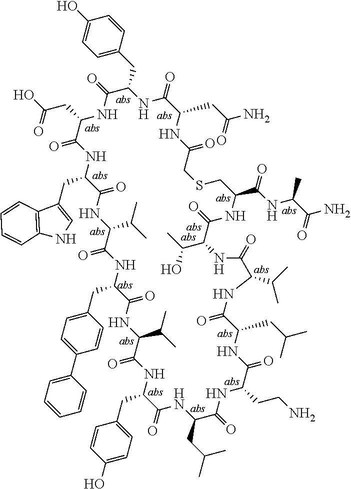 [SMILES: CC(C)C[C@@H]1NC(=O)[C@H](CCN)NC(=O)[C@@H](CC(C)C)NC(=O)[C@H](Cc2ccc(O)cc2)NC(=O)[C@H](C(C)C)NC(=O)[C@H](Cc2ccc(-c3ccccc3)cc2)NC(=O)[C@@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@@H]([C@@H](C)O)NC(=O)[C@H](C(C)C)NC1=O]

Compound 1031 was prepared on a 50 mol scale. The yield of the product was 5.6 mg, and its estimated purity by LCMS analysis was 100%. Analysis condition A: Retention time=1.71 min: ESI-MS(+) m/z [M+H]⁺: 1922.

### Preparation of Compound 1032

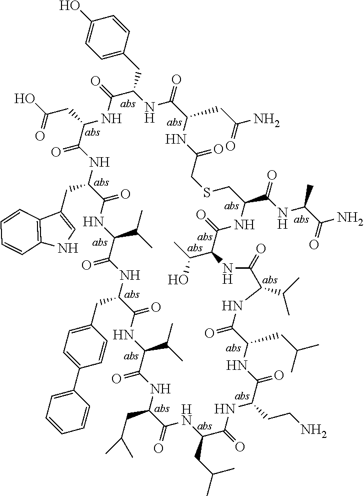 [SMILES: CC(C)C[C@@H]1NC(=O)[C@H](CCN)NC(=O)[C@@H](CC(C)C)NC(=O)[C@@H](CC(C)C)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1032 was prepared on a 50 μmol scale. The yield of the product was 2.6 mg, and its estimated purity by LCMS analysis was 88.8%. Analysis condition B: Retention time=1.82, 1.85 min; ESI-MS(+) m/z [M+H]⁺: 1870.86, 1870.86.

### Preparation of Compound 1033

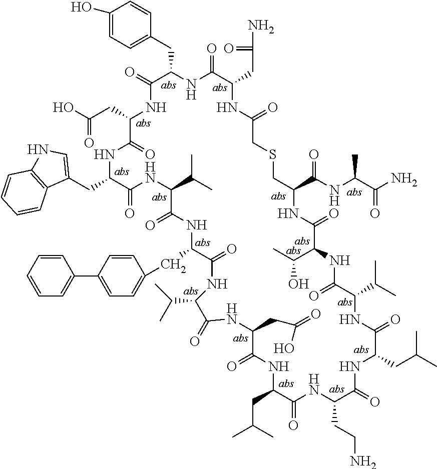 [SMILES: CC(C)C[C@@H]1NC(=O)[C@H](CCN)NC(=O)[C@@H](CC(C)C)NC(=O)[C@H](CC(=O)O)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1033 was prepared on a 50 μmol scale. The yield of the product was 4.1 mg, and its estimated purity by LCMS analysis was 91.7%. Analysis condition A: Retention time=1.62 min; ESI-MS(+) m/z [M+H]⁺: 1873.6.

### Preparation of Compound 1034

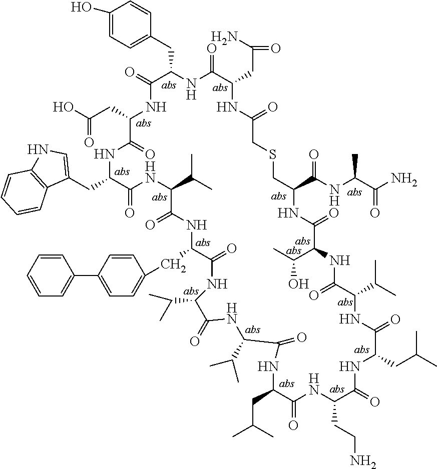 [SMILES: CC(C)C[C@@H]1NC(=O)[C@H](CCN)NC(=O)[C@@H](CC(C)C)NC(=O)[C@H](C(C)C)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1034 was prepared on a 50 μmol scale. The yield of the product was 1.1 mg, and its estimated purity by LCMS analysis was 92.3%. Analysis condition B: Retention time=1.88 min; ESI-MS(+) m/z [M+2H]²⁺: 929.1.

### Preparation of Compound 1035

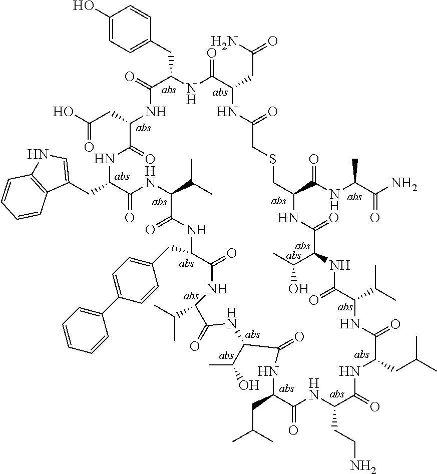 [SMILES: CC(C)C[C@@H]1NC(=O)[C@H](CCN)NC(=O)[C@@H](CC(C)C)NC(=O)[C@H]([C@@H](C)O)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1035 was prepared on a 50 μmol scale. The yield of the product was 3.8 mg, and its estimated purity by LCMS analysis was 97.7%. Analysis condition B: Retention time=1.79 min; ESI-MS(+) m/z [M+H]⁺: 1857.9.

### Preparation of Compound 1036

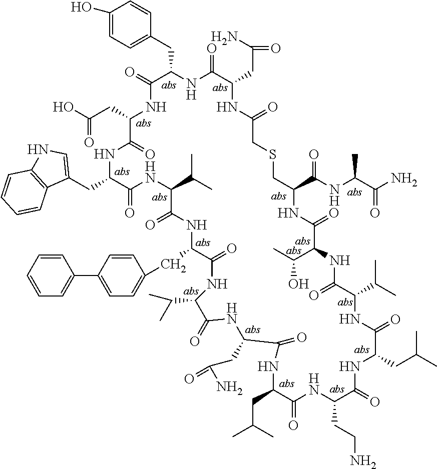 [SMILES: CC(C)C[C@@H]1NC(=O)[C@H](CCN)NC(=O)[C@@H](CC(C)C)NC(=O)[C@H](CC(N)=O)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1036 was prepared on a 50 μmol scale. The yield of the product was 8.9 mg, and its estimated purity by LCMS analysis was 99.3%. Analysis condition A: Retention time=1.61, 1.65 min; ESI-MS(+) m/z [M+H]⁺: 1873.24, 1873.24.

### Preparation of Compound 1037

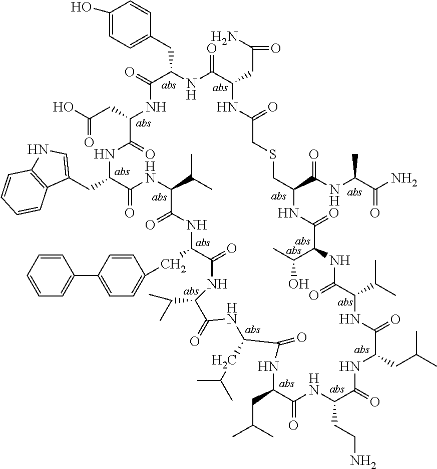 [SMILES: CC(C)C[C@@H]1NC(=O)[C@H](CCN)NC(=O)[C@@H](CC(C)C)NC(=O)[C@H](CC(C)C)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1037 was prepared on a 50 μmol scale. The yield of the product was 1.9 mg, and its estimated purity by LCMS analysis was 91%. Analysis condition A: Retention time=1.96 min; ESI-MS(+) m/z [M+H]⁺: 1871.2.

### Preparation of Compound 1038

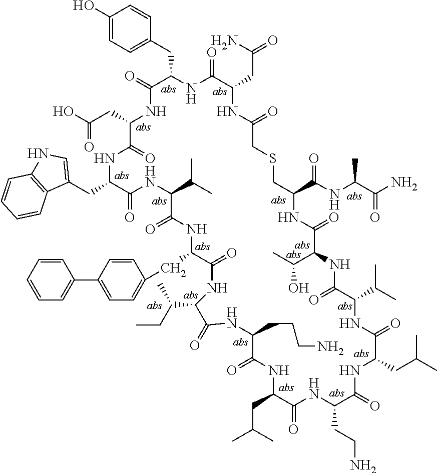 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1038 was prepared on a 50 μmol scale. The yield of the product was 7.1 mg, and its estimated purity by LCMS analysis was 97.8%. Analysis condition A: Retention time=1.7, 1.75 min; ESI-MS(+) m/z [M+H]⁺: 1886.3.

### Preparation of Compound 1039

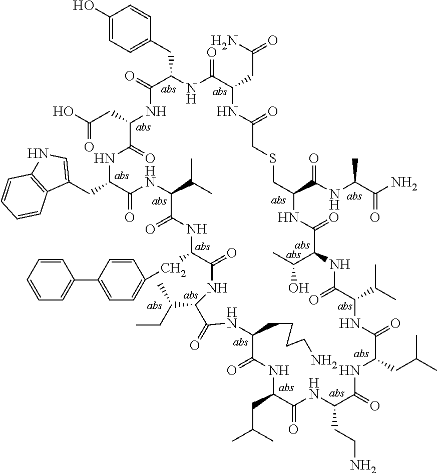 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCCN)NC1=O]

Compound 1039 was prepared on a 50 μmol scale. The yield of the product was 4.4 mg, and its estimated purity by LCMS analysis was 95.4%. Analysis condition B: Retention time=1.57 min; ESI-MS(+) m/z [M+2H]²⁺: 951.2.

### Preparation of Compound 1040

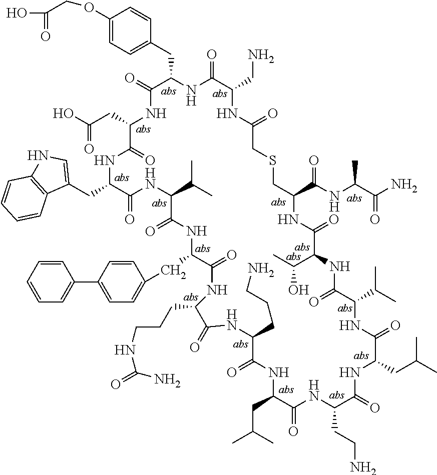 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1040 was prepared on a 50 μmol scale. The yield of the product was 5.1 mg, and its estimated purity by LCMS analysis was 92.4%. Analysis condition A: Retention time=1.38, 1.42 min; ESI-MS(+) m/z [M+H]⁺: 1960.

### Preparation of Compound 1041

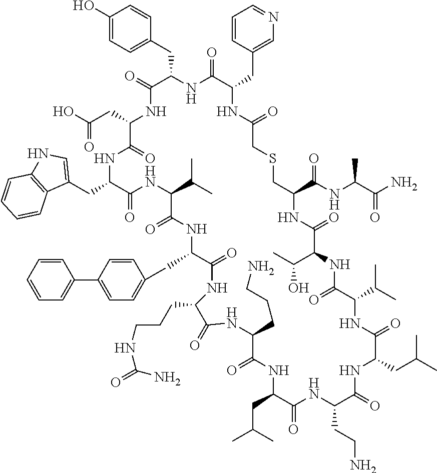 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC1=O]

Compound 1041 was prepared on a 50 μmol scale. The yield of the product was 4 mg, and its estimated purity by LCMS analysis was 97.8%. Analysis condition A: Retention time=1.54 min; ESI-MS(+) m/z [M+H]⁺: 1963.9.

### Preparation of Compound 1042

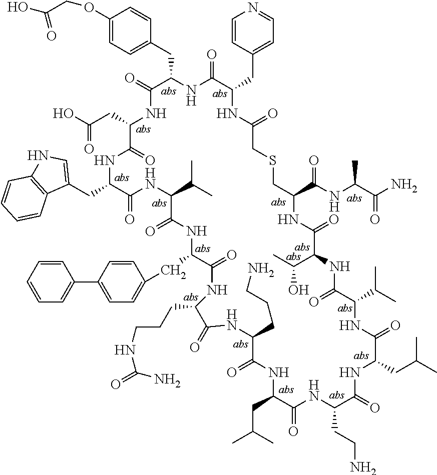 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1042 was prepared on a 50 μmol scale. The yield of the product was 3.7 mg, and its estimated purity by LCMS analysis was 90%. Analysis condition B: Retention time=1.27 min; ESI-MS(+) m/z [M+3H]²³+: 674.9.

### Preparation of Compound 1043

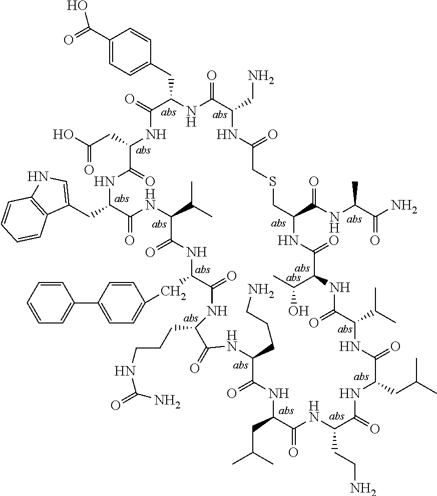 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1043 was prepared on a 50 μmol scale. The yield of the product was 4 mg, and its estimated purity by LCMS analysis was 91.1%. Analysis condition A: Retention time=1.4 min; ESI-MS(+) m/z [M+H]⁺: 1930.1.

### Preparation of Compound 1044

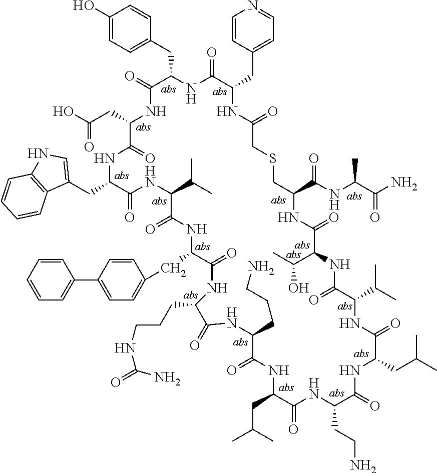 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1044 was prepared on a 50 mol scale. The yield of the product was 2.5 mg, and its estimated purity by LCMS analysis was 100%. Analysis condition A: Retention time=1.53 min; ESI-MS(+) m/z [M+H]⁺: 1964.2.

### Preparation of Compound 1045

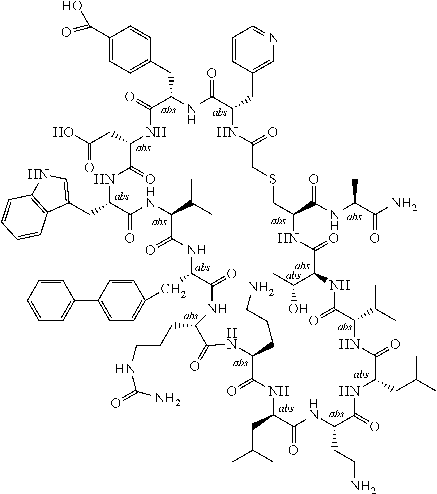 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC1=O]

Compound 1045 was prepared on a 50 μmol scale. The yield of the product was 2.3 mg, and its estimated purity by LCMS analysis was 97%. Analysis condition B: Retention time=1.4 min; ESI-MS(+) m/z [M+H]⁺: 1992.8.

### Preparation of Compound 1046

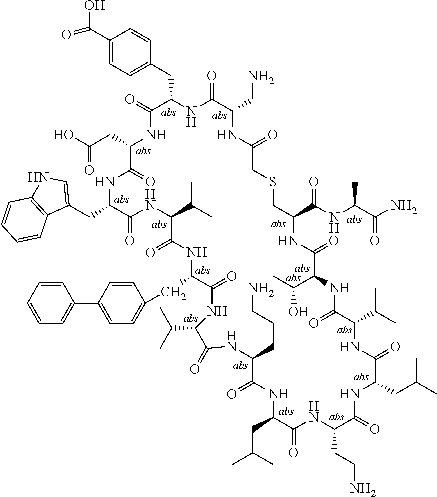 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1046 was prepared on a 50 μmol scale. The yield of the product was 2.6 mg, and its estimated purity by LCMS analysis was 88.4%. Analysis condition A: Retention time=1.47 min; ESI-MS(+) m/z [M+H]⁺: 1872.2.

### Preparation of Compound 1047

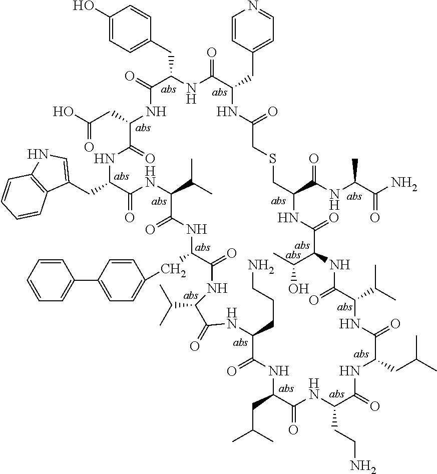 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1047 was prepared on a 50 μmol scale. The yield of the product was 2.4 mg, and its estimated purity by LCMS analysis was 100%. Analysis condition A: Retention time=1.66 min; ESI-MS(+) m/z [M+H]⁺: 1906.2.

### Preparation of Compound 1048

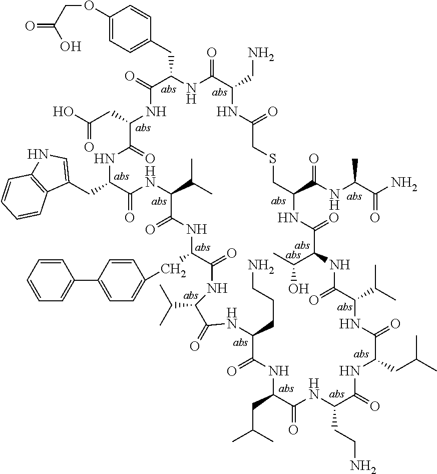 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1048 was prepared on a 50 mol scale. The yield of the product was 1.6 mg, and its estimated purity by LCMS analysis was 100%. Analysis condition A: Retention time=1.44 min; ESI-MS(+) m/z [M+2H]²⁺: 952.2.

### Preparation of Compound 1049

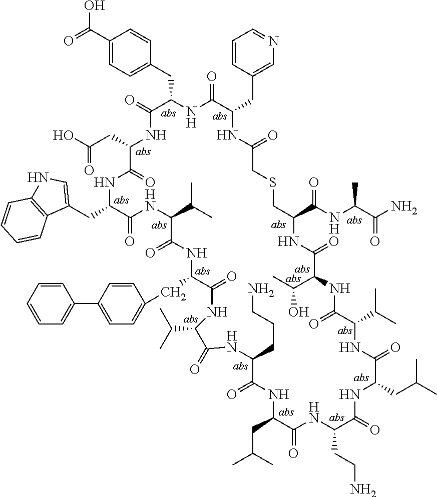 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC1=O]

Compound 1049 was prepared on a 50 μmol scale. The yield of the product was 1.6 mg, and its estimated purity by LCMS analysis was 100%. Analysis condition A: Retention time=1.55 min; ESI-MS(+) m/z [M+H]⁺: 1935.

### Preparation of Compound 1050

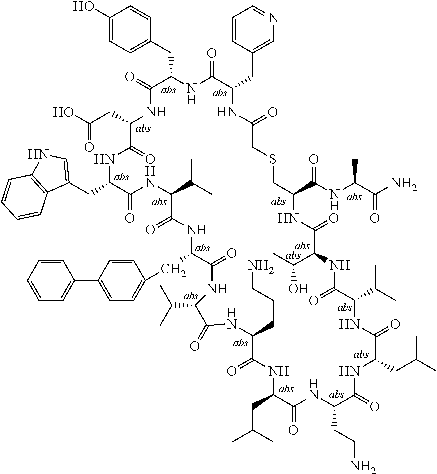 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC1=O]

Compound 1050 was prepared on a 50 μmol scale. The yield of the product was 1.8 mg, and its estimated purity by LCMS analysis was 100%. Analysis condition A: Retention time=1.64 min; ESI-MS(+) m/z [M+2H]²⁺: 954.2.

### Preparation of Compound 1051

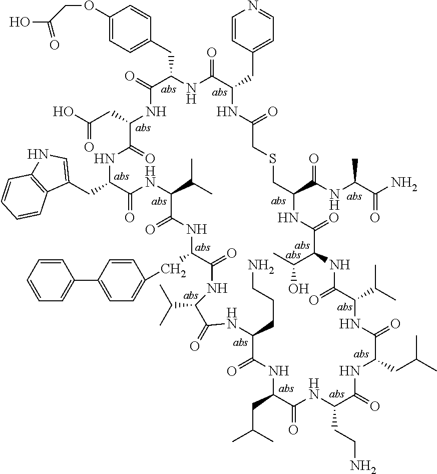 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1051 was prepared on a 50 μmol scale. The yield of the product was 1.9 mg, and its estimated purity by LCMS analysis was 100%. Analysis condition A: Retention time=1.54 min; ESI-MS(+) m/z [M+H]⁺: 1963.8.

### Preparation of Compound 1052

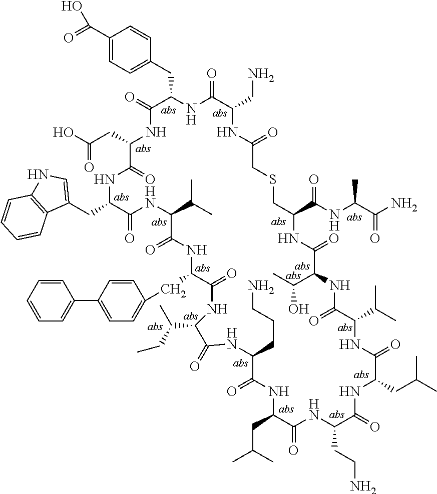 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1052 was prepared on a 50 mol scale. The yield of the product was 3.7 mg, and its estimated purity by LCMS analysis was 91.8%. Analysis condition B: Retention time=1.43 min; ESI-MS(+) m/z [M+H]⁺: 1886.2.

### Preparation of Compound 1053

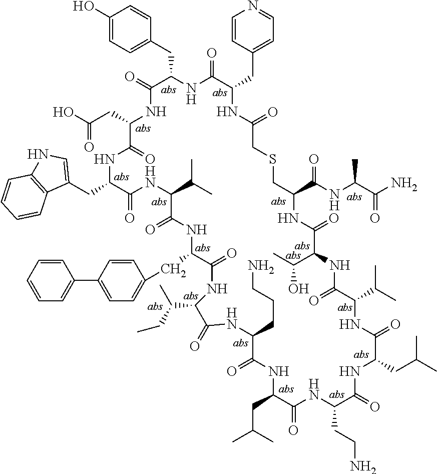 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1053 was prepared on a 50 μmol scale. The yield of the product was 2.2 mg, and its estimated purity by LCMS analysis was 92.7%. Analysis condition A: Retention time=1.73 min; ESI-MS(+) m/z [M+H]⁺: 1920.2.

### Preparation of Compound 1054

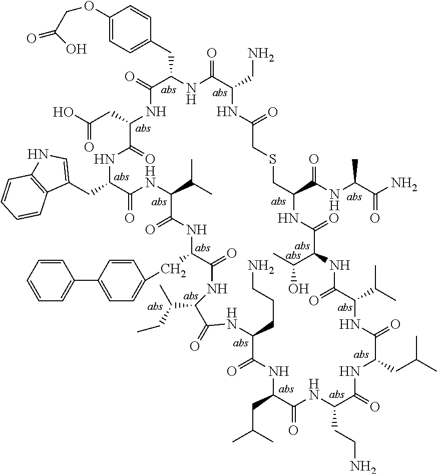 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1054 was prepared on a 50 μmol scale. The yield of the product was 5.4 mg, and its estimated purity by LCMS analysis was 96.7%. Analysis condition A: Retention time=1.53 min; ESI-MS(+) m/z [M+H]⁺: 1916.9.

### Preparation of Compound 1055

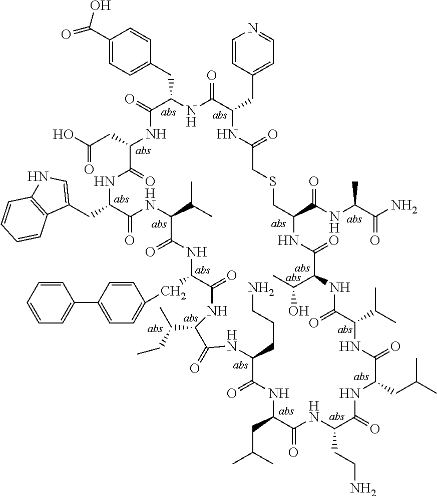 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1055 was prepared on a 50 μmol scale. The yield of the product was 3.8 mg, and its estimated purity by LCMS analysis was 82.3%. Analysis condition A: Retention time=1.58 min; ESI-MS(+) m/z [M+H]⁺: 1947.8.

### Preparation of Compound 1056

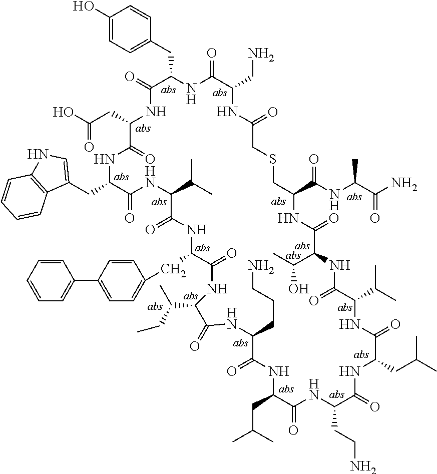 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1056 was prepared on a 50 μmol scale. The yield of the product was 2.1 mg, and its estimated purity by LCMS analysis was 87.8%. Analysis condition B: Retention time=1.29 min; ESI-MS(+) m/z [M+2H]²⁺: 930.1.

### Preparation of Compound 1057

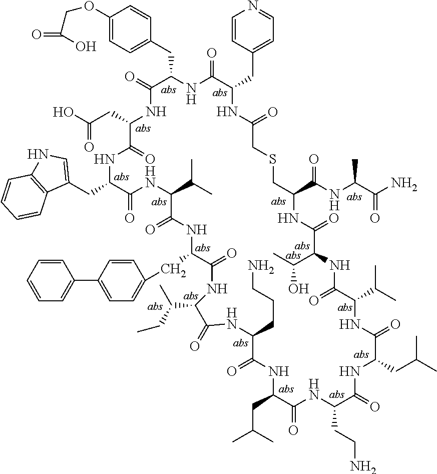 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1057 was prepared on a 50 μmol scale. The yield of the product was 1.8 mg, and its estimated purity by LCMS analysis was 97.6%. Analysis condition A: Retention time=1.46 min; ESI-MS(+) m/z [M+2H]²⁺: 989.9.

### Preparation of Compound 1058

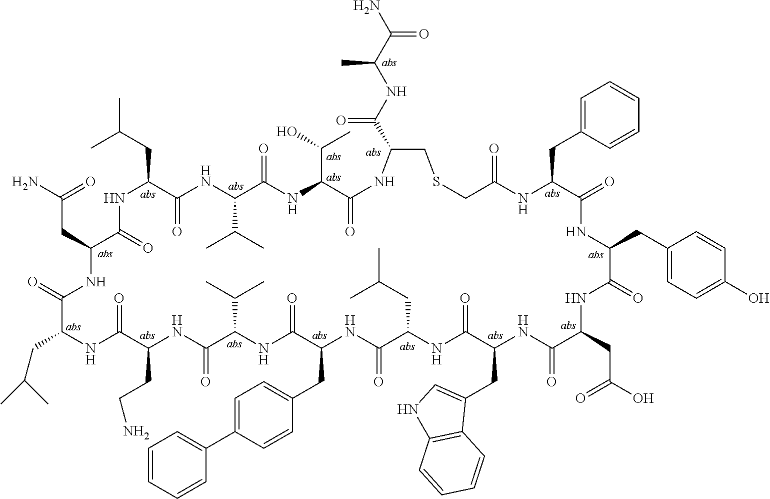 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1058 was prepared on a 50 mol scale. The yield of the product was 12.6 mg, and its estimated purity by LCMS analysis was 91.2%. Analysis condition A: Retention time=1.85 min; ESI-MS(+) m/z [M+2H]²⁺: 960.1.

### Preparation of Compound 1059

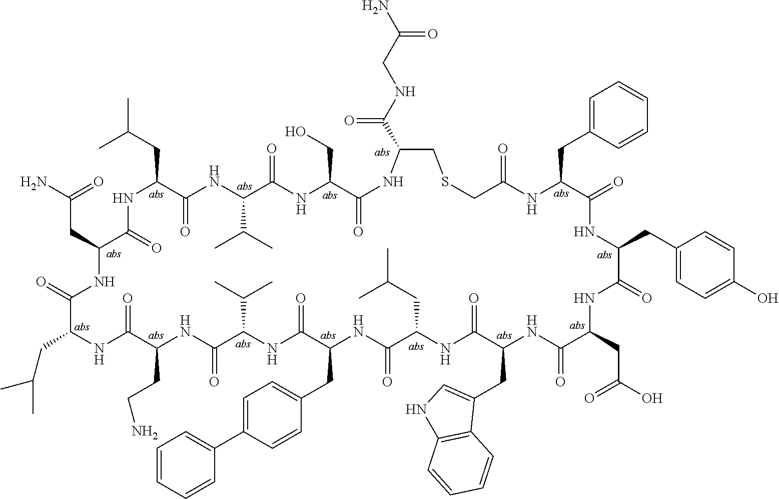 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1059 was prepared on a 50 mol scale. The yield of the product was 18.1 mg, and its estimated purity by LCMS analysis was 88.3%. Analysis condition B: Retention time=1.84 min; ESI-MS(+) m/z [M+H]⁺: 1892.3.

### Preparation of Compound 1060

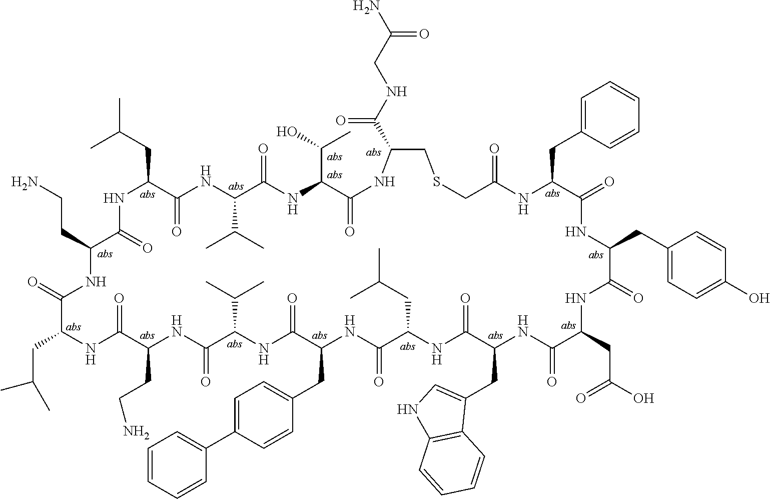 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1060 was prepared on a 50 μmol scale. The yield of the product was 5 mg, and its estimated purity by LCMS analysis was 92.5%. Analysis condition B: Retention time=1.59 min; ESI-MS(+) m/z [M+3H]³⁺: 631.

### Preparation of Compound 1061

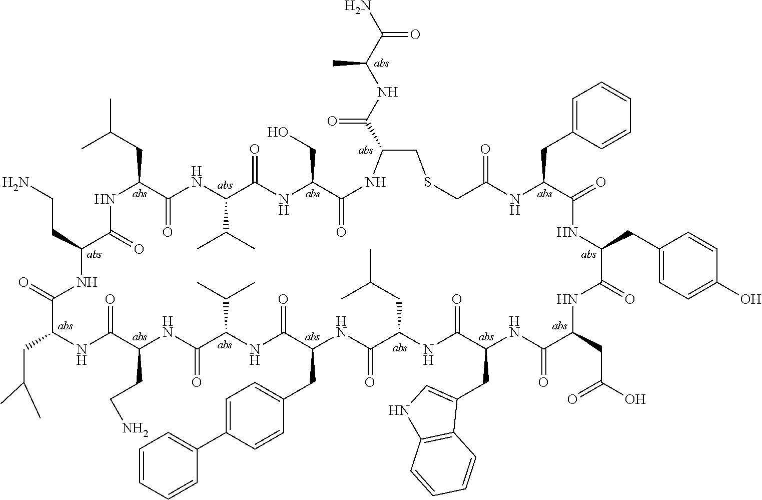 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1061 was prepared on a 50 μmol scale. The yield of the product was 7.6 mg, and its estimated purity by LCMS analysis was 91.6%. Analysis condition A: Retention time=1.85 min; ESI-MS(+) m/z [M+H]⁺: 1892.3.

### Preparation of Compound 1062

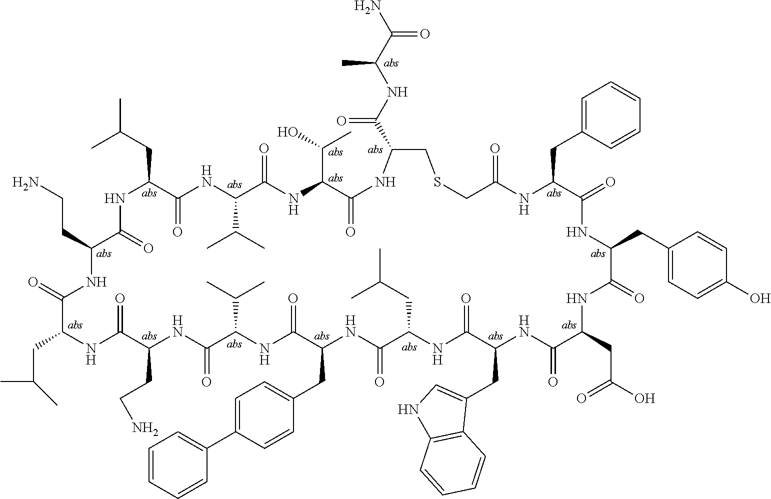 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1062 was prepared on a 50 μmol scale. The yield of the product was 9.7 mg, and its estimated purity by LCMS analysis was 97.7%. Analysis condition A: Retention time=1.82, 1.87 min; ESI-MS(+) m/z [M+H]⁺: 1906.

### Preparation of Compound 1063

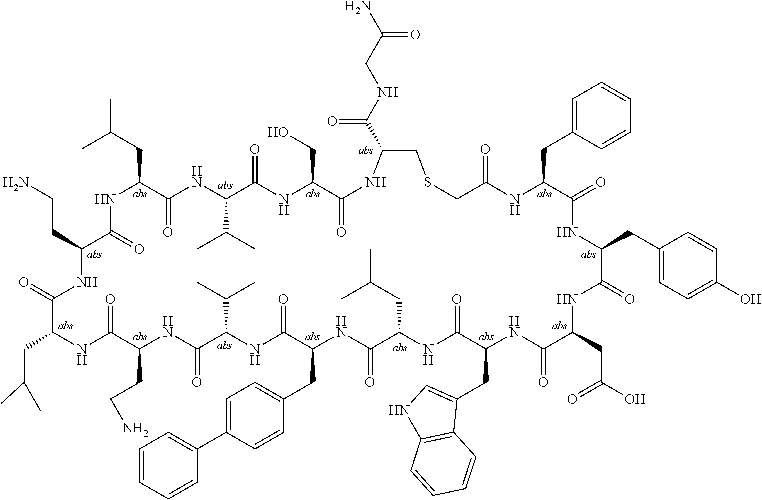 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC1=O]

Compound 1063 was prepared on a 50 μmol scale. The yield of the product was 16.6 mg, and its estimated purity by LCMS analysis was 90.6%. Analysis condition B: Retention time=1.57 min; ESI-MS(+) m/z [M+3H]³⁺: 626.3.

### Preparation of Compound 1064

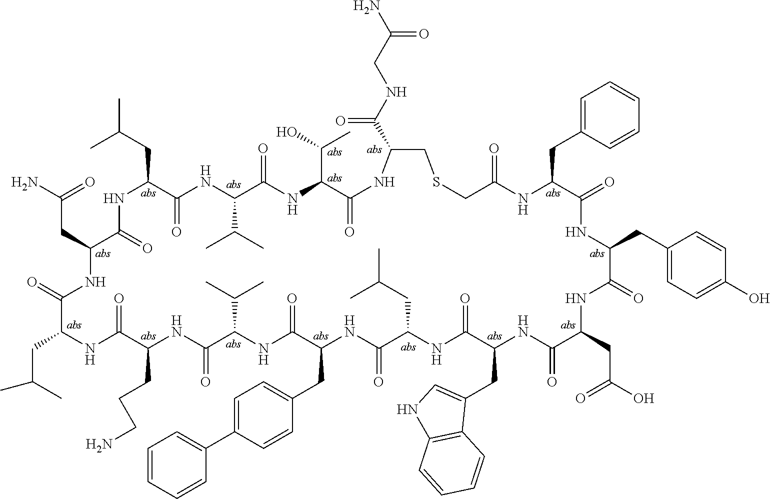 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1064 was prepared on a 50 mol scale. The yield of the product was 16 mg, and its estimated purity by LCMS analysis was 95.8%. Analysis condition B: Retention time=1.85 min; ESI-MS(+) m/z [M+H]⁺: 1920.2.

### Preparation of Compound 1065

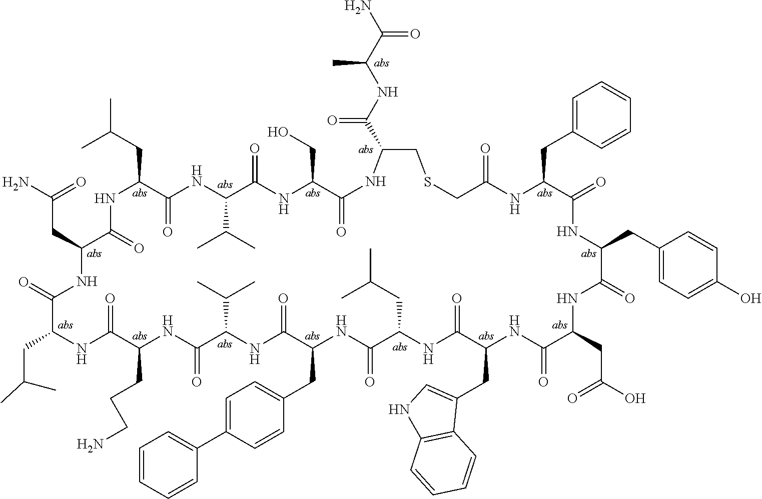 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1065 was prepared on a 50 μmol scale. The yield of the product was 5.7 mg, and its estimated purity by LCMS analysis was 98.5%. Analysis condition A: Retention time=1.78 min; ESI-MS(+) m/z [M+2H]²⁺: 960.5.

### Preparation of Compound 1066

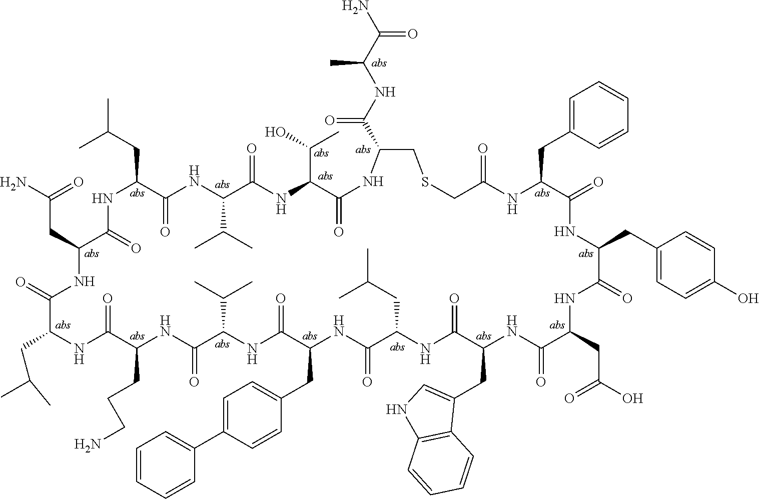 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1066 was prepared on a 50 mol scale. The yield of the product was 8.3 mg, and its estimated purity by LCMS analysis was 97.9%. Analysis condition B: Retention time=1.88 min; ESI-MS(+) m/z [M+H]⁺: 1934.2.

### Preparation of Compound 1067

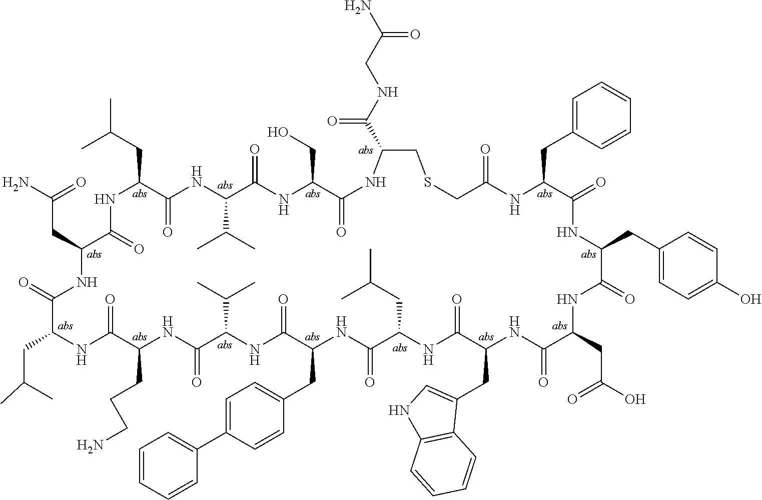 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1067 was prepared on a 50 mol scale. The yield of the product was 3.5 mg, and its estimated purity by LCMS analysis was 97.5%. Analysis condition B: Retention time=1.73 min: ESI-MS(+) m/z [M+2H]²⁺: 953.3.

### Preparation of Compound 1068

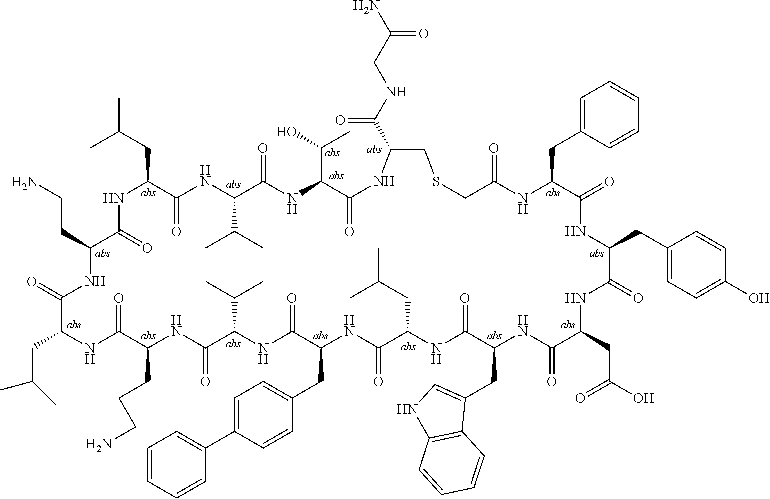 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1068 was prepared on a 50 mol scale. The yield of the product was 15 mg, and its estimated purity by LCMS analysis was 98.3%. Analysis condition A: Retention time=1.84 min; ESI-MS(+) m/z [M+2H]²⁺: 953.1.

### Preparation of Compound 1069

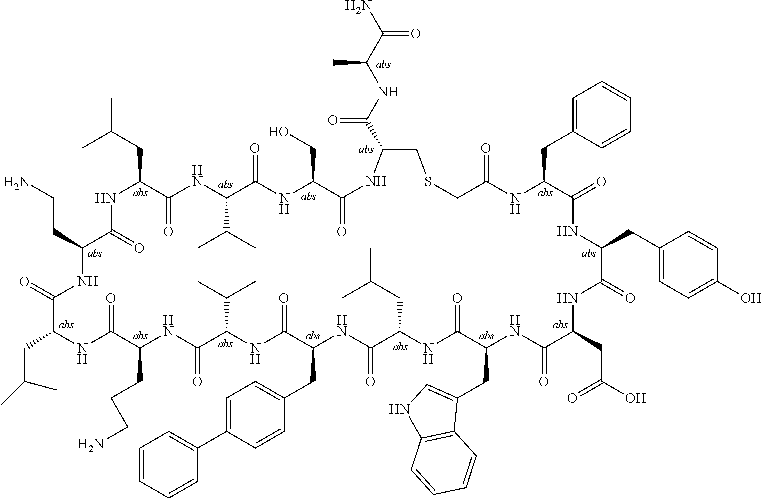 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1069 was prepared on a 50 mol scale. The yield of the product was 3.5 mg, and its estimated purity by LCMS analysis was 81.6%. Analysis condition A: Retention time=1.84 min; ESI-MS(+) m/z [M+H]⁺: 1905.1.

### Preparation of Compound 1070

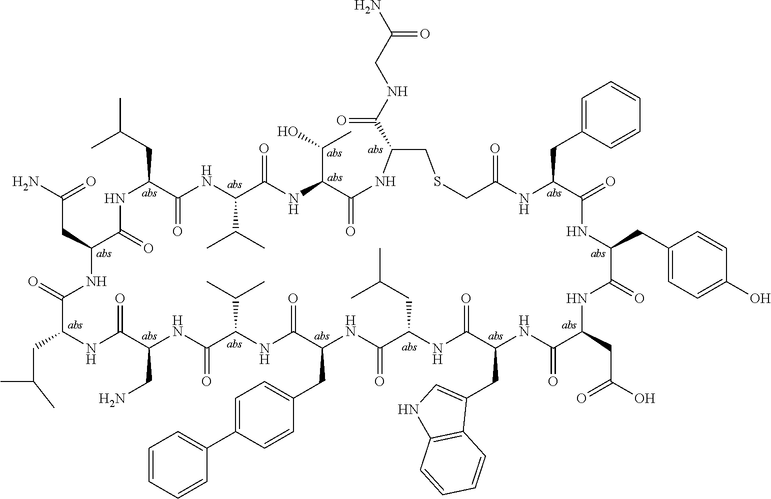 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1070 was prepared on a 50 mol scale. The yield of the product was 11.6 mg, and its estimated purity by LCMS analysis was 94.5%. Analysis condition A: Retention time=1.87 min; ESI-MS(+) m/z [M+2H]²⁺: 946.1.

### Preparation of Compound 1071

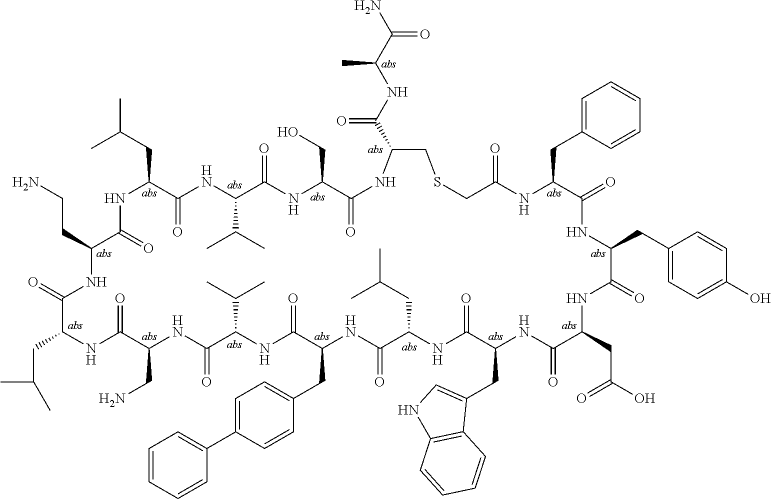 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1071 was prepared on a 50 μmol scale. The yield of the product was 8.1 mg, and its estimated purity by LCMS analysis was 97.5%. Analysis condition A: Retention time=1.86 min; ESI-MS(+) m/z [M+2H]²⁺: 946.1.

### Preparation of Compound 1072

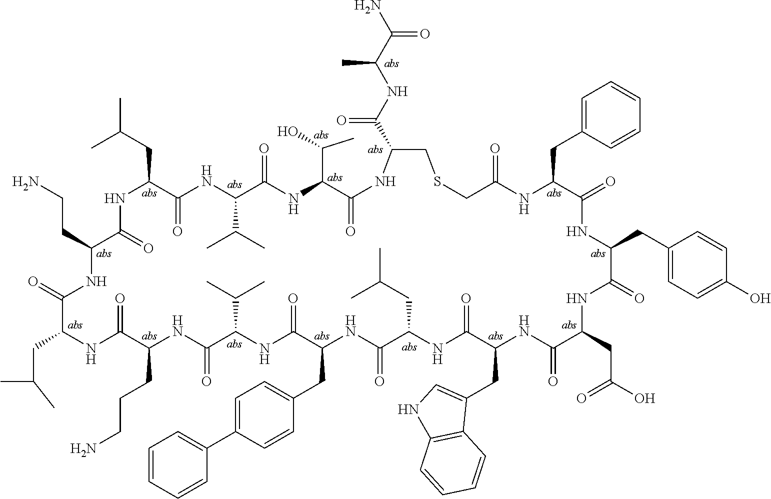 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1072 was prepared on a 50 μmol scale. The yield of the product was 6.5 mg, and its estimated purity by LCMS analysis was 95.1%. Analysis condition B: Retention time=1.61 min; ESI-MS(+) m/z [M+2H]²⁺: 960.2.

### Preparation of Compound 1073

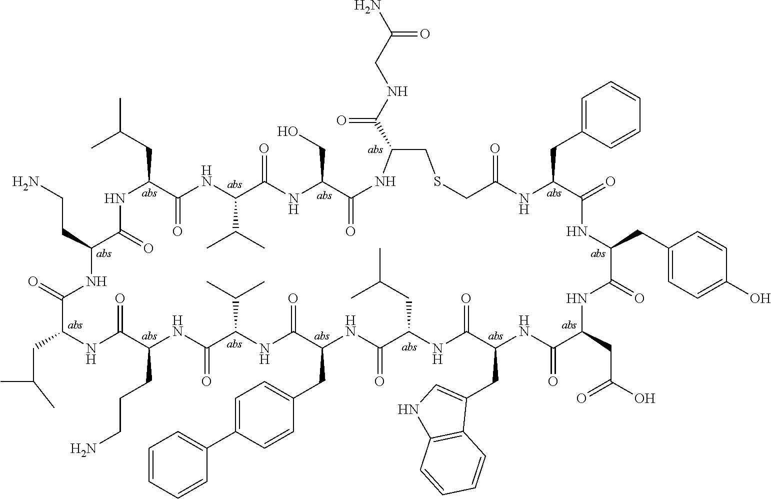 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1073 was prepared on a 50 mol scale. The yield of the product was 5.2 mg, and its estimated purity by LCMS analysis was 97.9%. Analysis condition A: Retention time=1.82 min; ESI-MS(+) m/z [M+2H]²⁺: 946.1.

### Preparation of Compound 1074

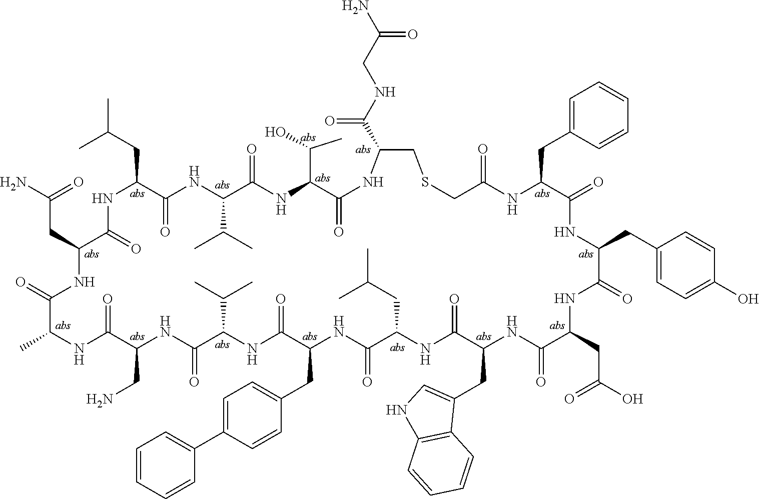 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1074 was prepared on a 50 μmol scale. The yield of the product was 17.9 mg, and its estimated purity by LCMS analysis was 96.5%. Analysis condition A: Retention time=1.73 min; ESI-MS(+) m/z [M+2H]²⁺: 925.2.

### Preparation of Compound 1075

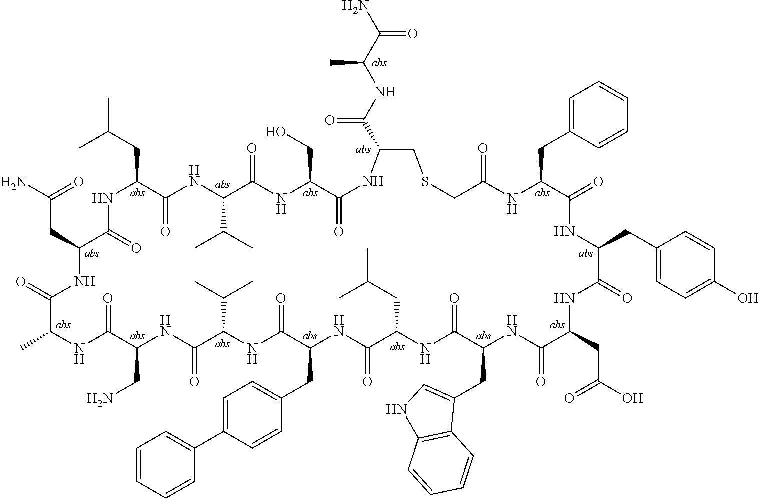 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1075 was prepared on a 50 μmol scale. The yield of the product was 11.2 mg, and its estimated purity by LCMS analysis was 81.8%. Analysis condition B: Retention time=1.65 min; ESI-MS(+) m/z [M+2H]²⁺: 925.1.

### Preparation of Compound 1076

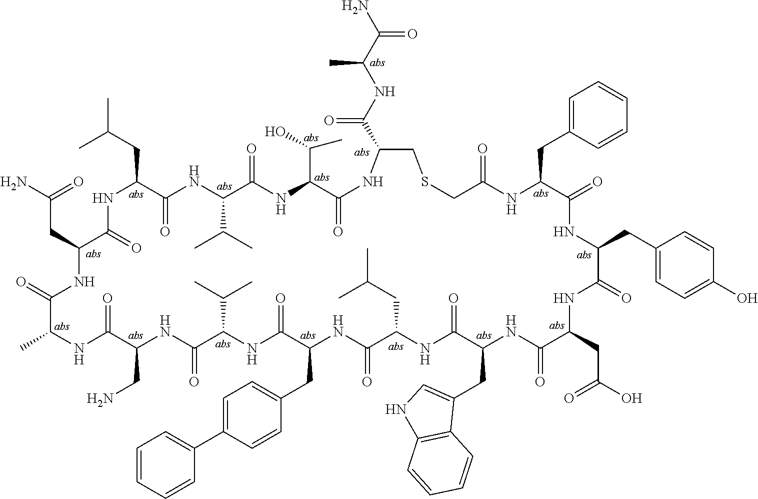 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1076 was prepared on a 50 μmol scale. The yield of the product was 13 mg, and its estimated purity by LCMS analysis was 95.7%. Analysis condition A: Retention time=1.75 min; ESI-MS(+) m/z [M+2H]²⁺: 932.1.

### Preparation of Compound 1077

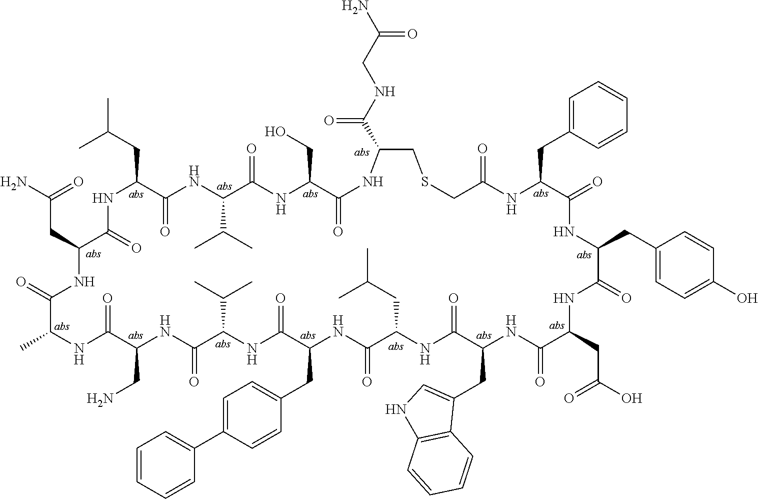 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1077 was prepared on a 50 μmol scale. The yield of the product was 12.8 mg, and its estimated purity by LCMS analysis was 80.4%. Analysis condition B: Retention time=1.64 min; ESI-MS(+) m/z [M+2H]²⁺: 918.1.

### Preparation of Compound 1078

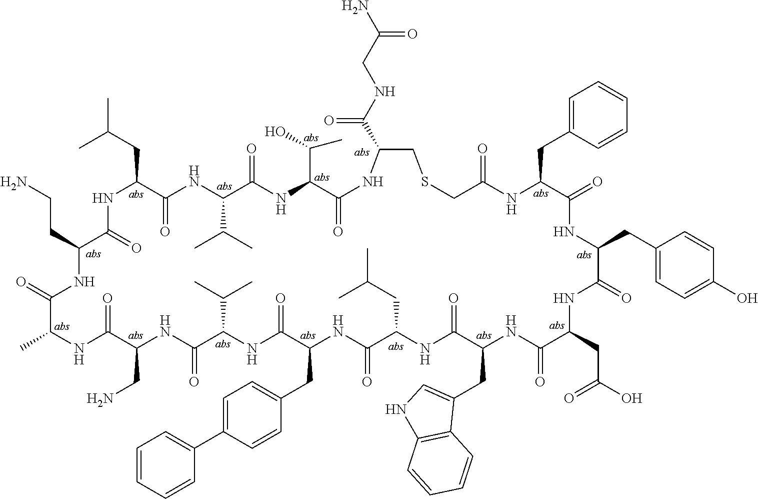 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1078 was prepared on a 50 μmol scale. The yield of the product was 11.3 mg, and its estimated purity by LCMS analysis was 97.6%. Analysis condition B: Retention time=1.49 min; ESI-MS(+) m/z [M+2H]²⁺: 918.1.

### Preparation of Compound 1079

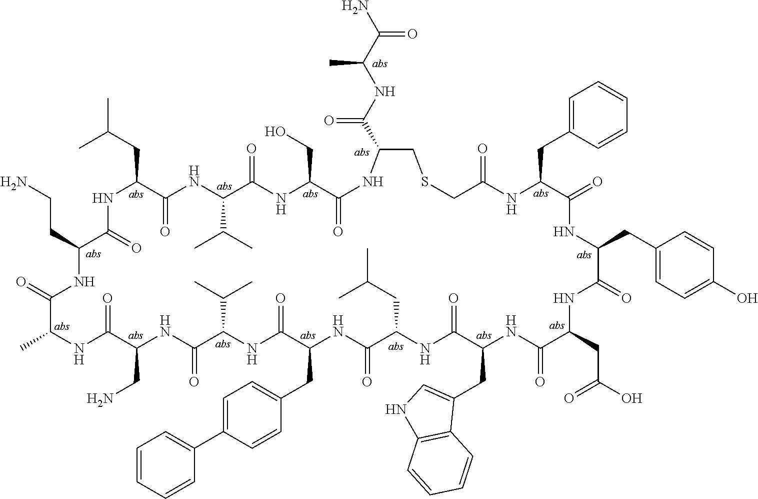 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1079 was prepared on a 50 mol scale. The yield of the product was 9 mg, and its estimated purity by LCMS analysis was 82.5%. Analysis condition B: Retention time=1.56 min; ESI-MS(+) m/z [M+2H]²⁺: 918.4.

### Preparation of Compound 1080

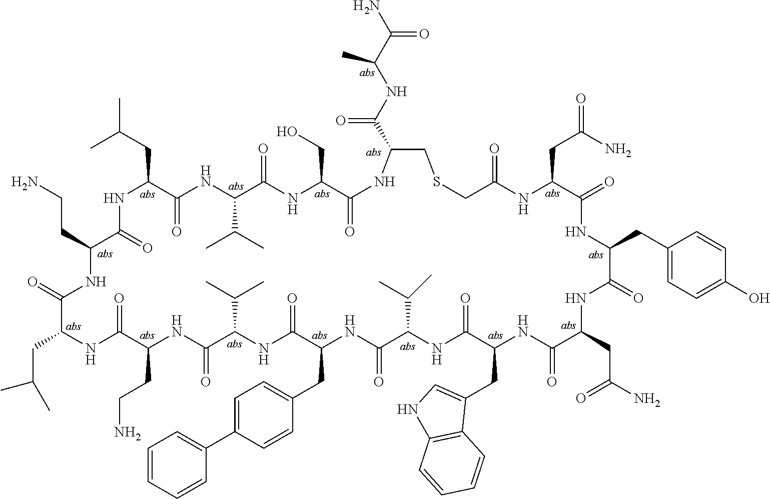 [SMILES: CC(C)C[C@@H]1NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1080 was prepared on a 50 mol scale. The yield of the product was 2.4 mg, and its estimated purity by LCMS analysis was 83.2%. Analysis condition B: Retention time=1.46, 1.5 min; ESI-MS(+) m/z [M+H]⁺: 1844.

### Preparation of Compound 1081

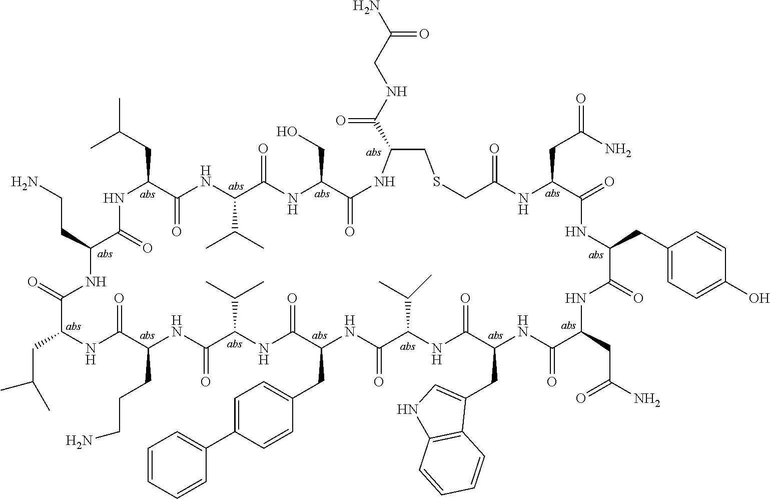 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1081 was prepared on a 50 mol scale. The yield of the product was 1 mg, and its estimated purity by LCMS analysis was 86.6%. Analysis condition B: Retention time=1.32 min; ESI-MS(+) m/z [M+2H]²⁺: 922.1.

### Preparation of Compound 1082

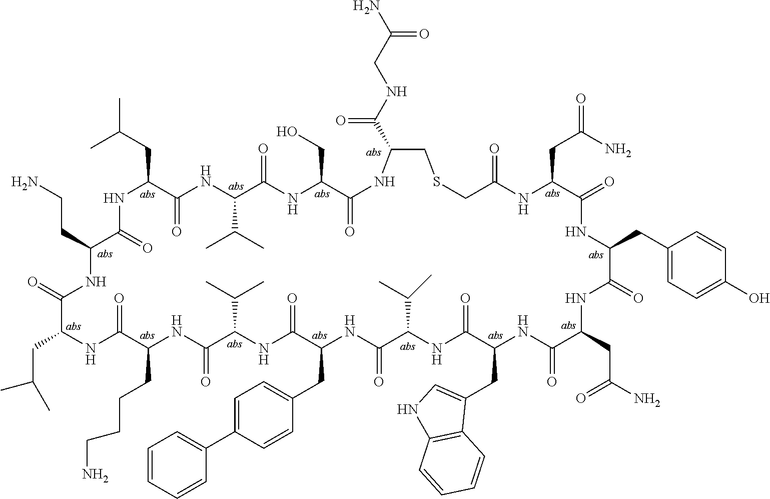 [SMILES: CC(C)C[C@@H]1NC(=O)[C@H](CCN)NC(=O)[C@@H](CC(C)C)NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1082 was prepared on a 50 μmol scale. The yield of the product was 3 mg, and its estimated purity by LCMS analysis was 87.4%. Analysis condition B: Retention time=1.49 min; ESI-MS(+) m/z [M+H]⁺: 1857.2.

### Preparation of Compound 1083

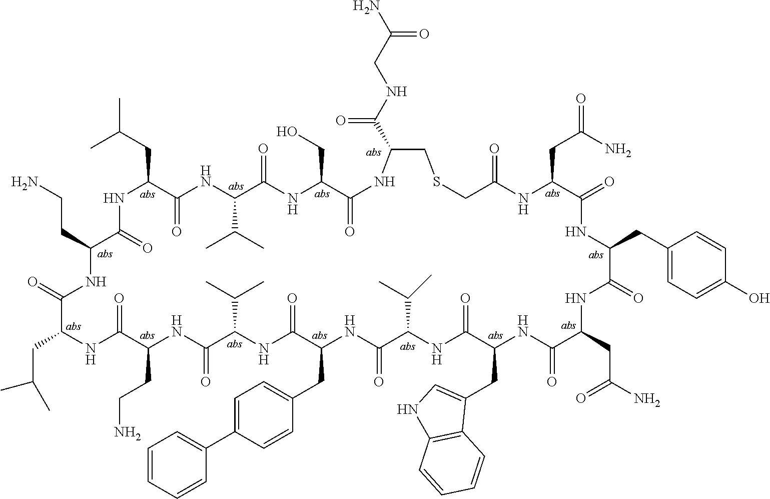 [SMILES: CC(C)C[C@@H]1NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1083 was prepared on a 50 μmol scale. The yield of the product was 2.1 mg, and its estimated purity by LCMS analysis was 88.5%. Analysis condition B: Retention time=1.3 min; ESI-MS(+) m/z [M+2H]²⁺: 915.

### Preparation of Compound 1084

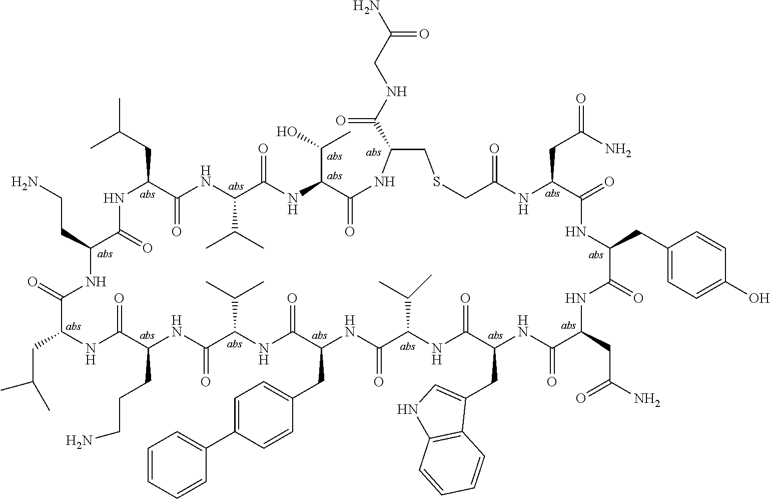 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1084 was prepared on a 50 mol scale. The yield of the product was 3.6 mg, and its estimated purity by LCMS analysis was 89.6%. Analysis condition B: Retention time=1.4 min; ESI-MS(+) m/z [M+2H]²⁺: 929.2.

### Preparation of Compound 1085

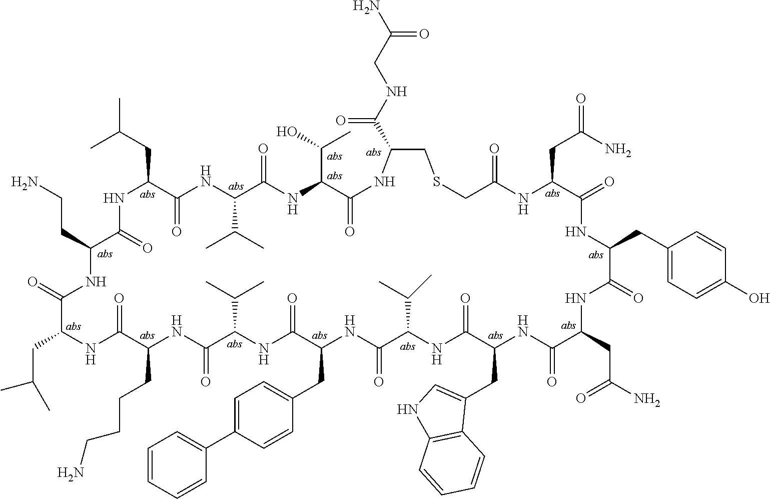 [SMILES: CC(C)C[C@@H]1NC(=O)[C@H](CCN)NC(=O)[C@@H](CC(C)C)NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1085 was prepared on a 50 mol scale. The yield of the product was 3.6 mg, and its estimated purity by LCMS analysis was 92.2%. Analysis condition A: Retention time=1.6 min; ESI-MS(+) m/z [M+2H]²⁺: 936.1.

### Preparation of Compound 1086

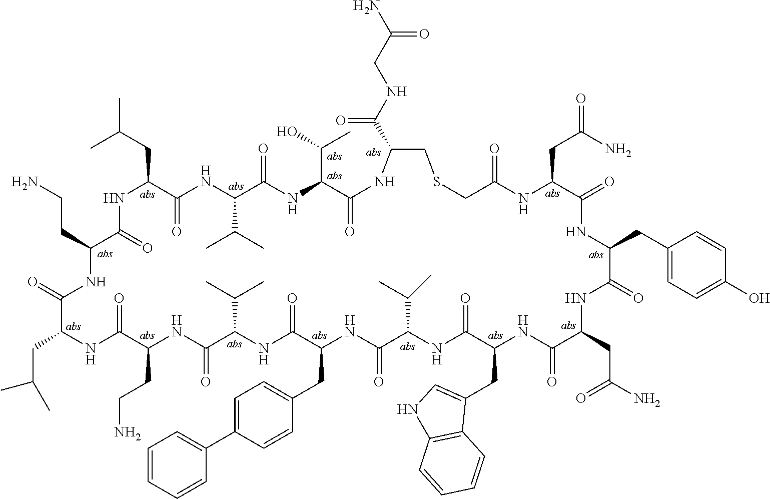 [SMILES: CC(C)C[C@@H]1NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1086 was prepared on a 50 μmol scale. The yield of the product was 3.6 mg, and its estimated purity by LCMS analysis was 91.5%. Analysis condition B: Retention time=1.39, 1.43 min; ESI-MS(+) m/z [M+H]⁺: 1843.2.

### Preparation of Compound 1087

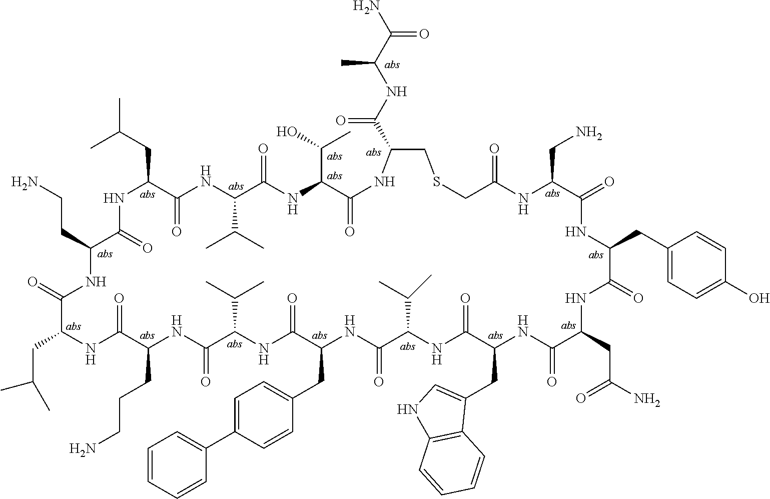 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1087 was prepared on a 50 μmol scale. The yield of the product was 14.1 mg, and its estimated purity by LCMS analysis was 86.4%. Analysis condition B: Retention time=1.3 min; ESI-MS(+) m/z [M+3H]³⁺: 615.1.

### Preparation of Compound 1088

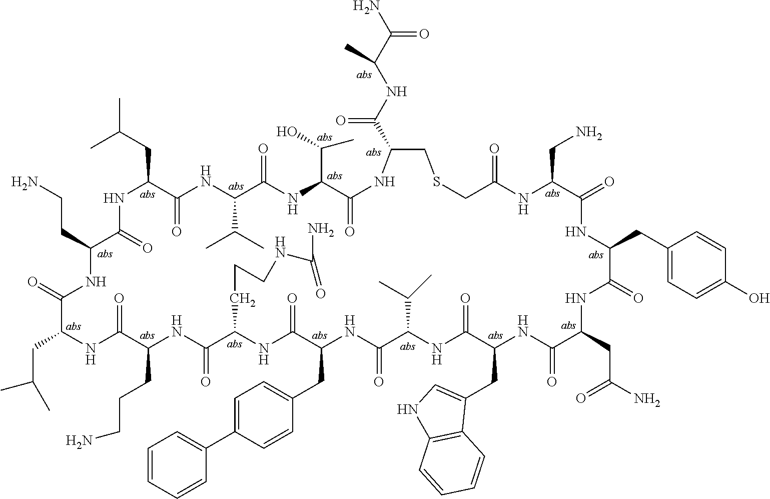 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1088 was prepared on a 50 μmol scale. The yield of the product was 4 mg, and its estimated purity by LCMS analysis was 93.6%. Analysis condition B: Retention time=1.28 min; ESI-MS(+) m/z [M+2H]²⁺: 951.1.

### Preparation of Compound 1089

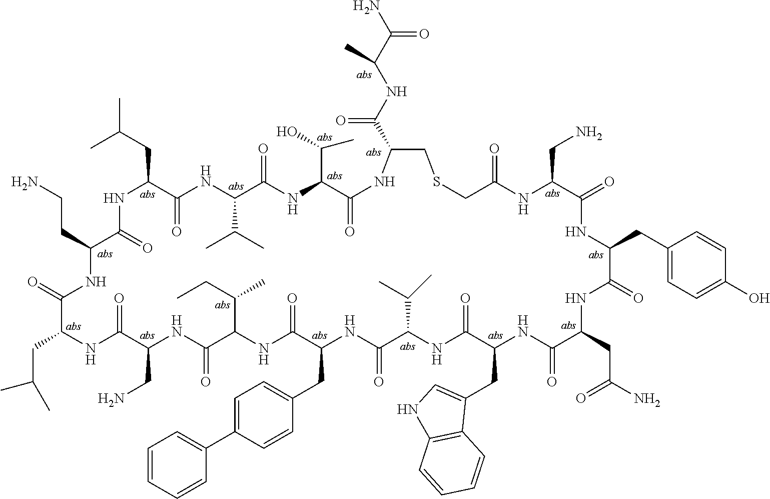 [SMILES: CC[C@H](C)C1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CN)NC1=O]

Compound 1089 was prepared on a 50 μmol scale. The yield of the product was 9.6 mg, and its estimated purity by LCMS analysis was 90.2%. Analysis condition A: Retention time=1.75 min; ESI-MS(+) m/z [M+2H]²⁺: 915.1.

### Preparation of Compound 1090

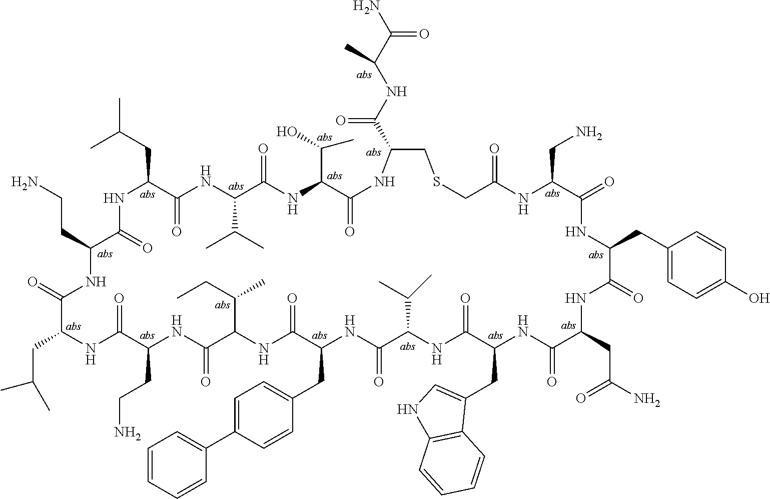 [SMILES: CC[C@H](C)C1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 1090 was prepared on a 50 μmol scale. The yield of the product was 5.8 mg, and its estimated purity by LCMS analysis was 94.6%. Analysis condition B: Retention time=1.31 min; ESI-MS(+) m/z [M+3H]³⁺: 615.

### Preparation of Compound 1091

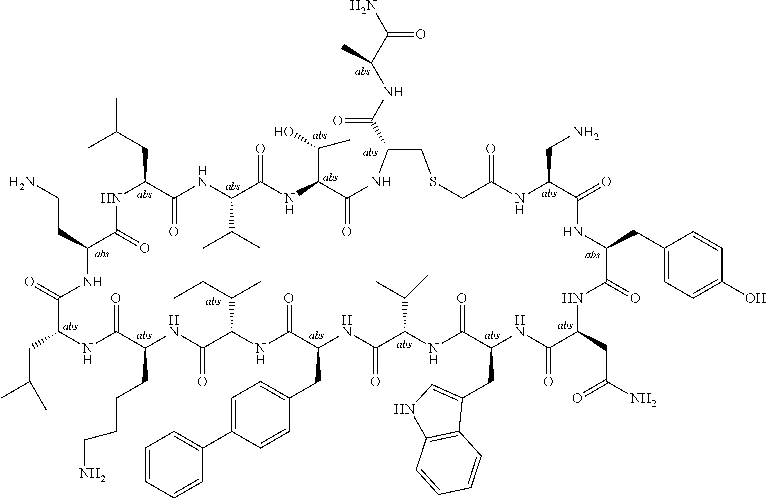 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(N)=O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCCN)NC1=O]

Compound 1091 was prepared on a 50 μmol scale. The yield of the product was 4.2 mg, and its estimated purity by LCMS analysis was 92.7%. Analysis condition A: Retention time=1.53 min; ESI-MS(+) m/z [M+2H]²⁺: 936.3.

### Preparation of Compound 1092

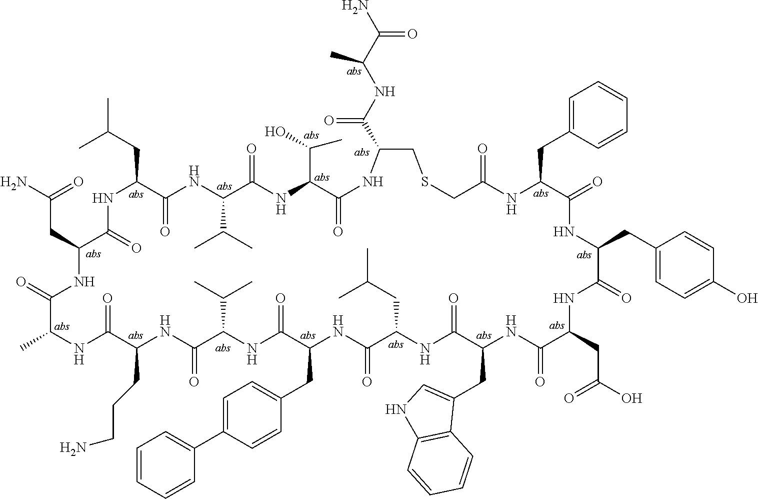 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1092 was prepared on a 50 μmol scale. The yield of the product was 14.1 mg, and its estimated purity by LCMS analysis was 92.9%. Analysis condition A: Retention time=1.73 min; ESI-MS(+) m/z [M+2H]²⁺: 946.1.

### Preparation of Compound 1093

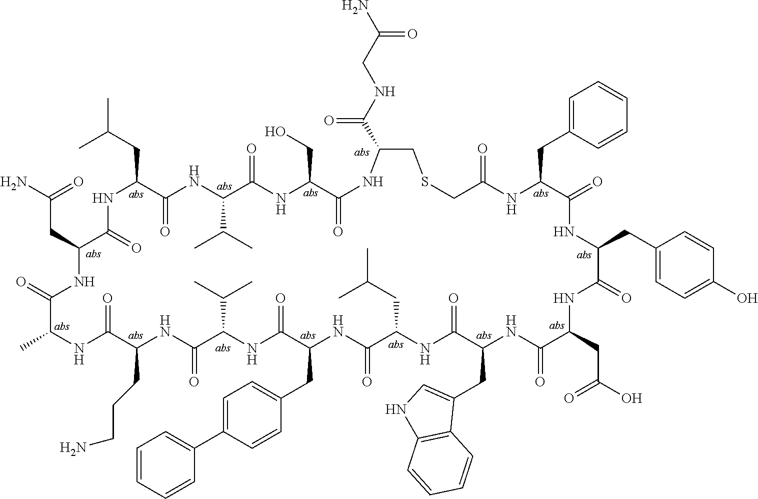 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1093 was prepared on a 50 μmol scale. The yield of the product was 12.7 mg, and its estimated purity by LCMS analysis was 87.9%. Analysis condition B: Retention time=1.73 min; ESI-MS(+) m/z [M+2H]⁺: 932.2.

### Preparation of Compound 1094

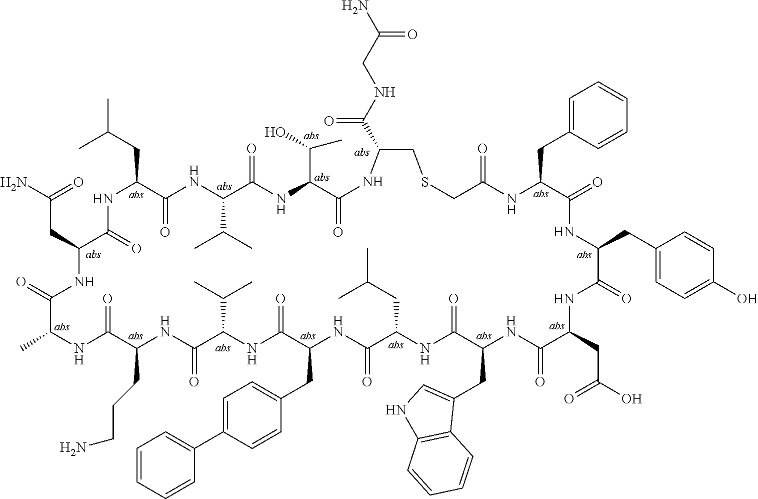 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1094 was prepared on a 50 μmol scale. The yield of the product was 16 mg, and its estimated purity by LCMS analysis was 91.1%. Analysis condition A: Retention time=1.75 min; ESI-MS(+) m/z [M+2H]²⁺: 932.1.

### Preparation of Compound 1095

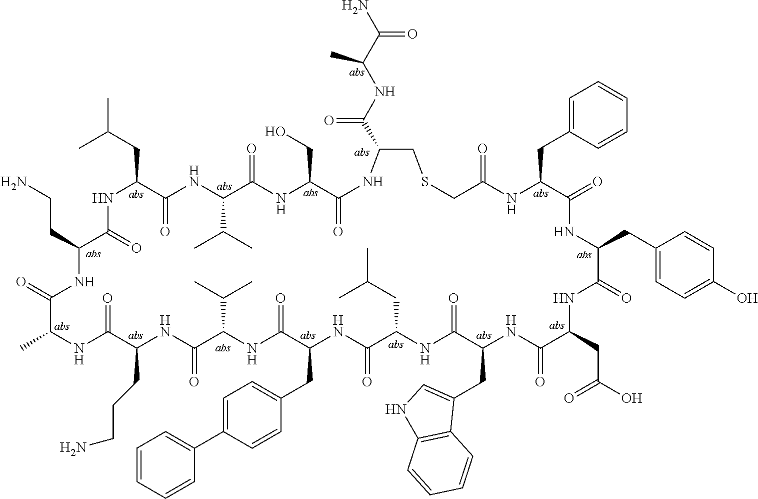 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1095 was prepared on a 50 μmol scale. The yield of the product was 11 mg, and its estimated purity by LCMS analysis was 87.8%. Analysis condition B: Retention time=1.49 min; ESI-MS(+) m/z [M+2H]²⁺: 932.1.

### Preparation of Compound 1096

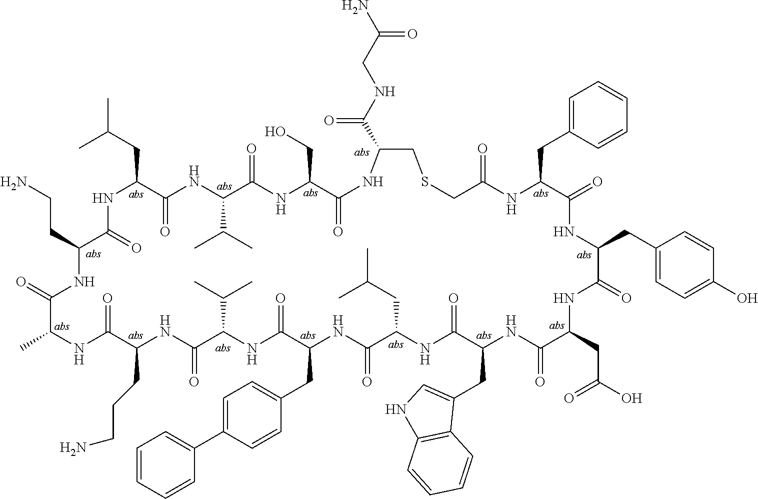 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1096 was prepared on a 50 mol scale. The yield of the product was 18 mg, and its estimated purity by LCMS analysis was 86.8%. Analysis condition B: Retention time=1.6 min; ESI-MS(+) m/z [M+H]⁺: 1850.3.

### Preparation of Compound 1097

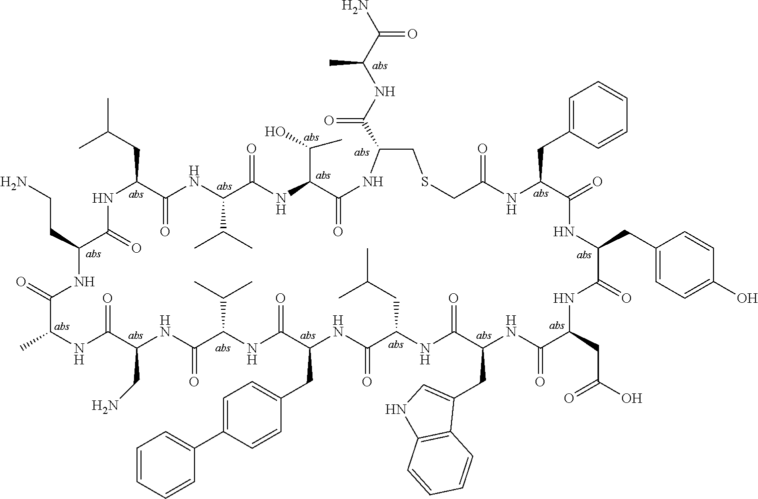 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1097 was prepared on a 50 μmol scale. The yield of the product was 8.8 mg, and its estimated purity by LCMS analysis was 91.5%. Analysis condition A: Retention time=1.63 min; ESI-MS(+) m/z [M+2H]²⁺: 924.9.

### Preparation of Compound 1098

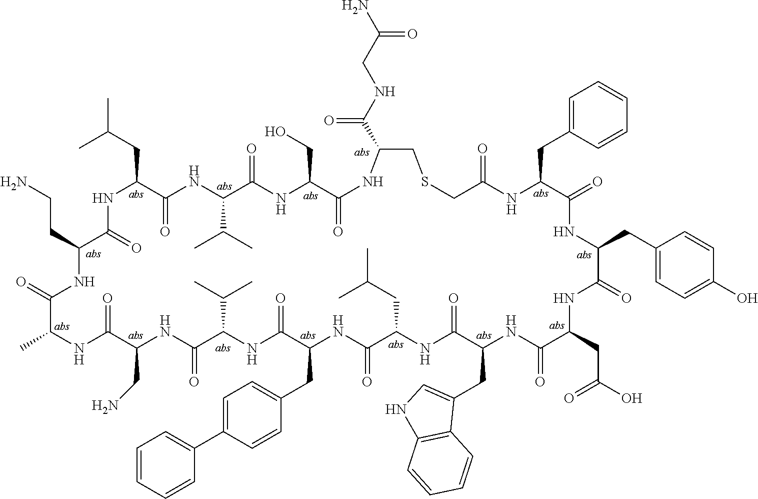 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CN)NC(=O)[C@H](C(C)C)NC(=O)[C@H](Cc2ccc(-c3ccccc3)cc2)NC1=O]

Compound 1098 was prepared on a 50 μmol scale. The yield of the product was 5.8 mg, and its estimated purity by LCMS analysis was 95.4%. Analysis condition A: Retention time=1.7 min; ESI-MS(+) m/z [M+2H]²⁺: 911.1.

### Preparation of Compound 1099

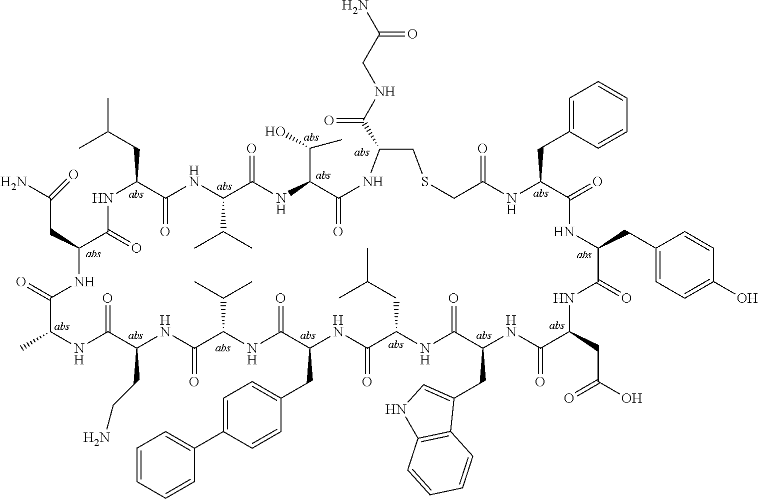 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1099 was prepared on a 50 μmol scale. The yield of the product was 11.7 mg, and its estimated purity by LCMS analysis was 92.2%. Analysis condition B: Retention time=1.6 min; ESI-MS(+) m/z [M+2H]²⁺: 932.2.

### Preparation of Compound 1100

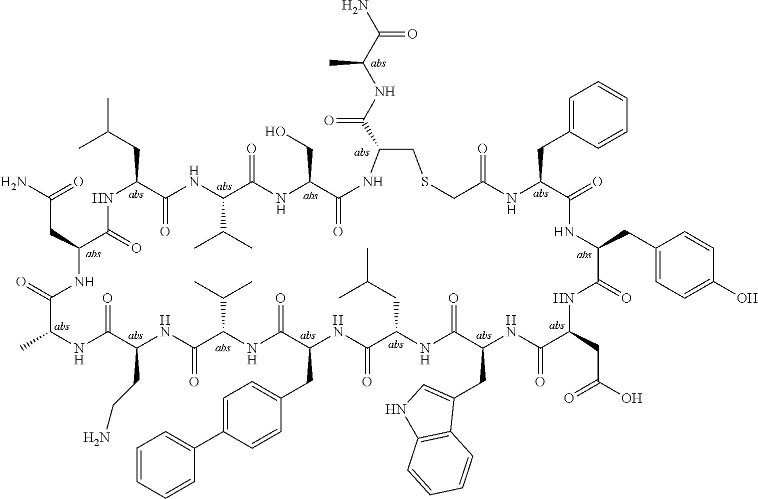 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1100 was prepared on a 50 μmol scale. The yield of the product was 11.5 mg, and its estimated purity by LCMS analysis was 94.4%. Analysis condition B: Retention time=1.6 min; ESI-MS(+) m/z [M+2H]²⁺: 932.

### Preparation of Compound 1101

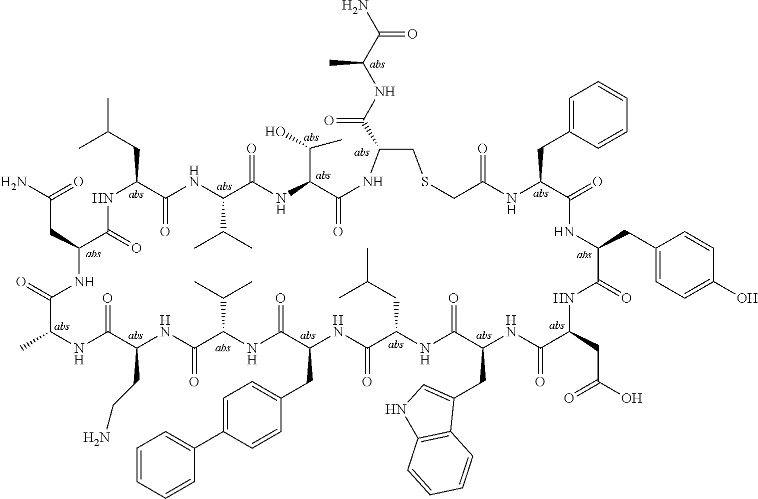 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1101 was prepared on a 50 μmol scale. The yield of the product was 10.7 mg, and its estimated purity by LCMS analysis was 94.6%. Analysis condition A: Retention time=1.73 min; ESI-MS(+) m/z [M+2H]²⁺: 939.

### Preparation of Compound 1102

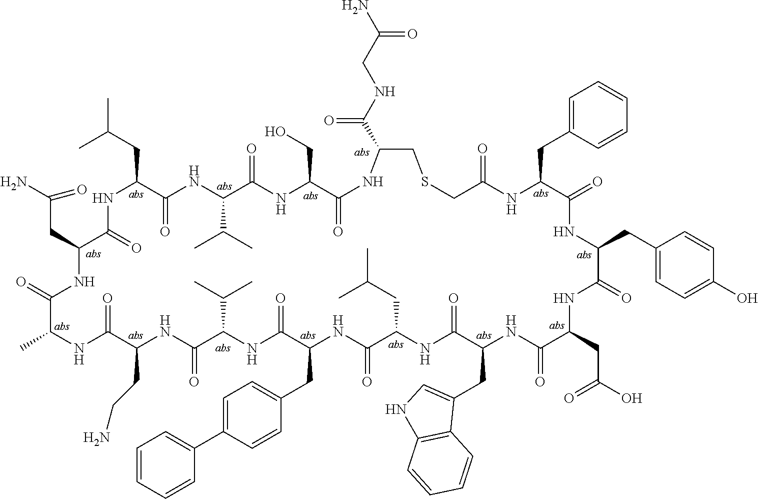 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1102 was prepared on a 50 mol scale. The yield of the product was 15.7 mg, and its estimated purity by LCMS analysis was 91.6%. Analysis condition B: Retention time=1.59 min; ESI-MS(+) m/z [M+2H]²⁺: 925.

### Preparation of Compound 1103

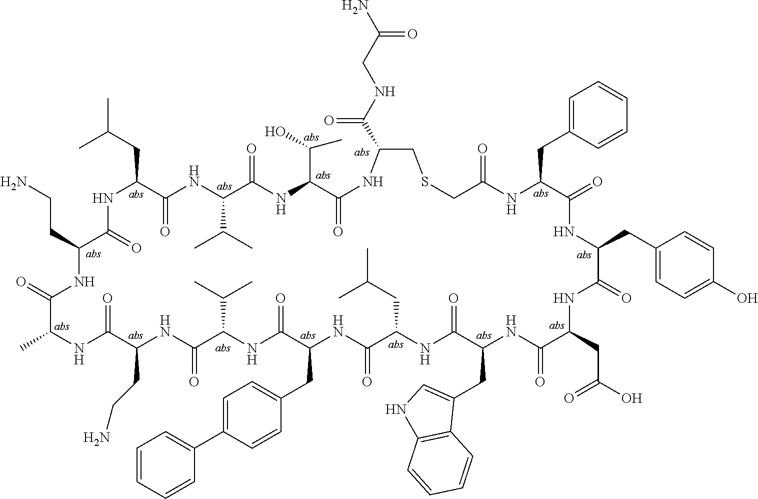 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1103 was prepared on a 50 μmol scale. The yield of the product was 12.3 mg, and its estimated purity by LCMS analysis was 93.1%. Analysis condition A: Retention time=1.65 min; ESI-MS(+) m/z [M+2H]²⁺: 925.2.

### Preparation of Compound 1104

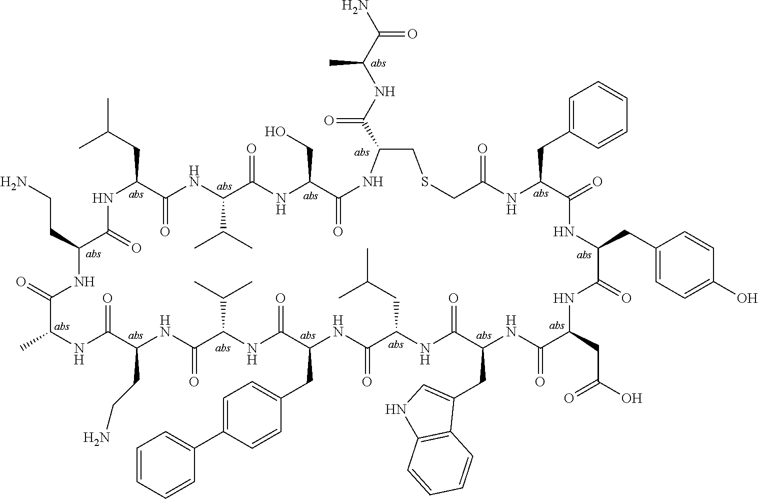 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1104 was prepared on a 50 μmol scale. The yield of the product was 8.2 mg, and its estimated purity by LCMS analysis was 91.4%. Analysis condition A: Retention time=1.59 min; ESI-MS(+) m/z [M+2H]²⁺: 924.9.

### Preparation of Compound 1105

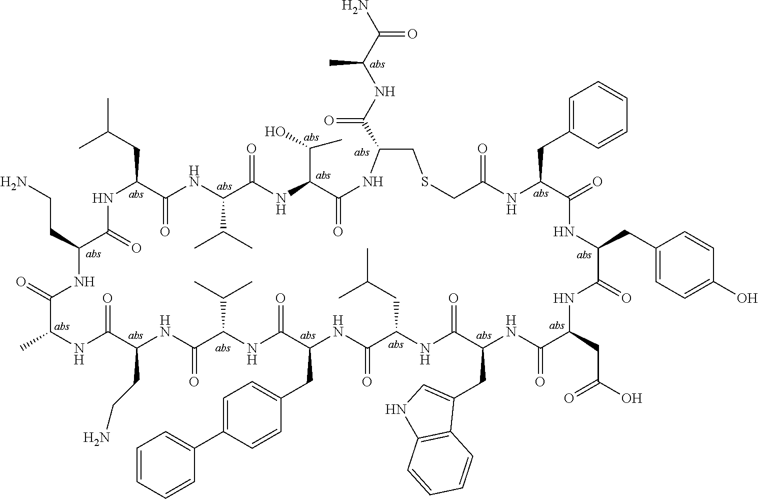 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1105 was prepared on a 50 μmol scale. The yield of the product was 8.8 mg, and its estimated purity by LCMS analysis was 93.9%. Analysis condition B: Retention time=1.47, 1.49 min; ESI-MS(+) m/z [M+2H]²⁺: 932.23, 932.23.

### Preparation of Compound 1106

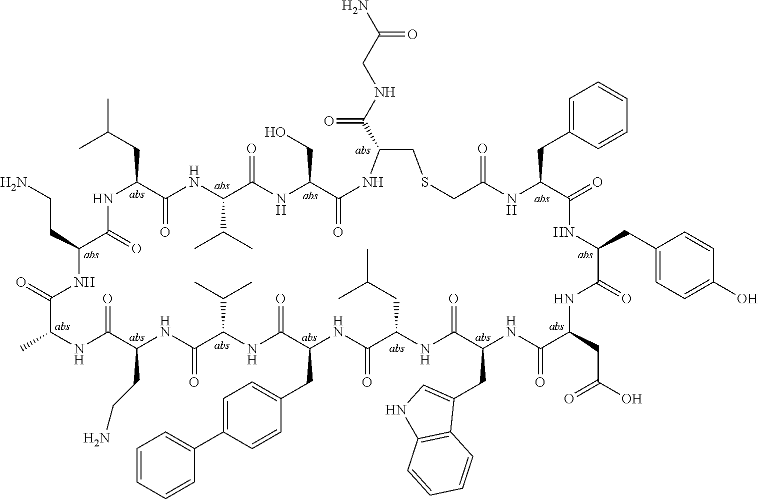 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](C)NC(=O)[C@H](CCN)NC(=O)[C@H](C(C)C)NC(=O)[C@H](Cc2ccc(-c3ccccc3)cc2)NC1=O]

Compound 1106 was prepared on a 50 μmol scale. The yield of the product was 11.3 mg, and its estimated purity by LCMS analysis was 93.1%. Analysis condition A: Retention time=1.6 min; ESI-MS(+) m/z [M+2H]²⁺: 918.1.

### Preparation of Compound 1107

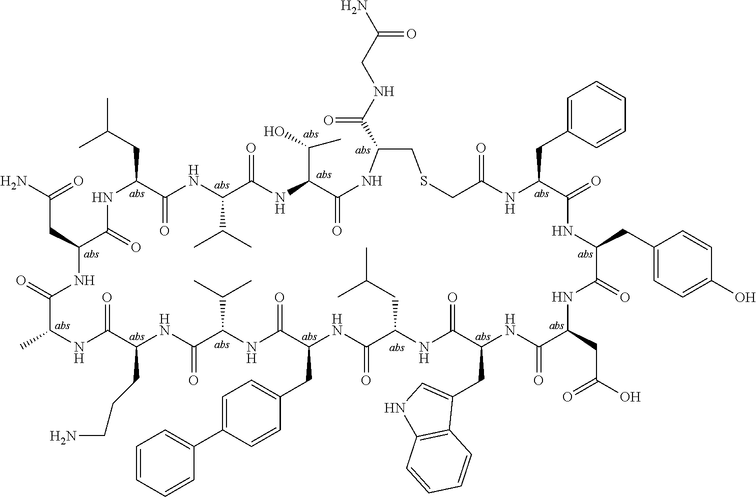 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CC(N)=O)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1107 was prepared on a 50 μmol scale. The yield of the product was 16 mg, and its estimated purity by LCMS analysis was 97.9%. Analysis condition A: Retention time=1.71 min; ESI-MS(+) m/z [M+2H]²⁺: 939.

### Preparation of Compound 1108

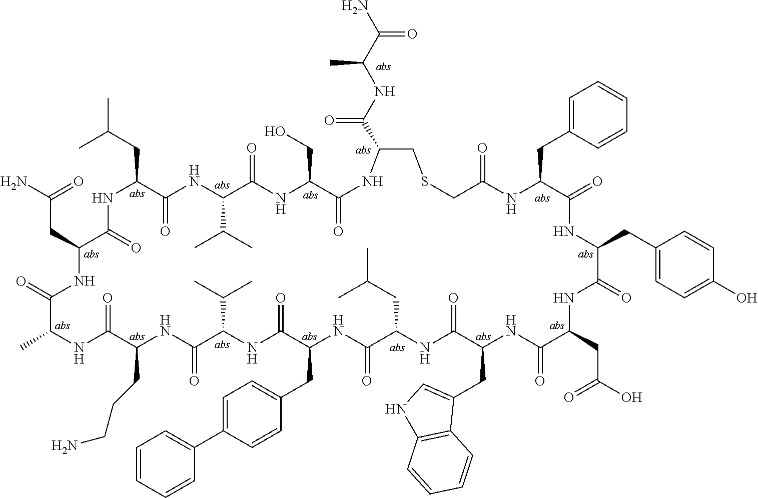 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](C)NC(=O)[C@H](CCCN)NC(=O)[C@H](C(C)C)NC(=O)[C@H](Cc2ccc(-c3ccccc3)cc2)NC1=O]

Compound 1108 was prepared on a 50 μmol scale. The yield of the product was 5.9 mg, and its estimated purity by LCMS analysis was 96.9%. Analysis condition B: Retention time=1.65 min; ESI-MS(+) m/z [M+2H]²⁺: 939.1.

### Preparation of Compound 1109

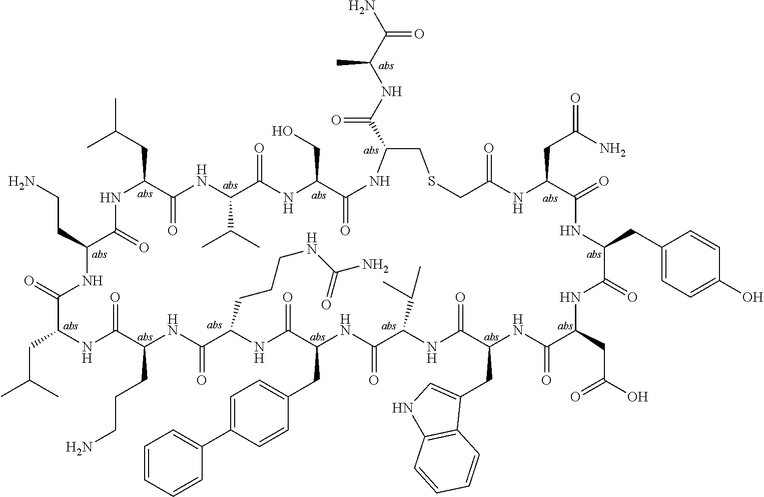 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1109 was prepared on a 50 μmol scale. The yield of the product was 6.4 mg, and its estimated purity by LCMS analysis was 95.9%. Analysis condition B: Retention time=1.3 min; ESI-MS(+) m/z [M+2H]²⁺: 959.

### Preparation of Compound 1110

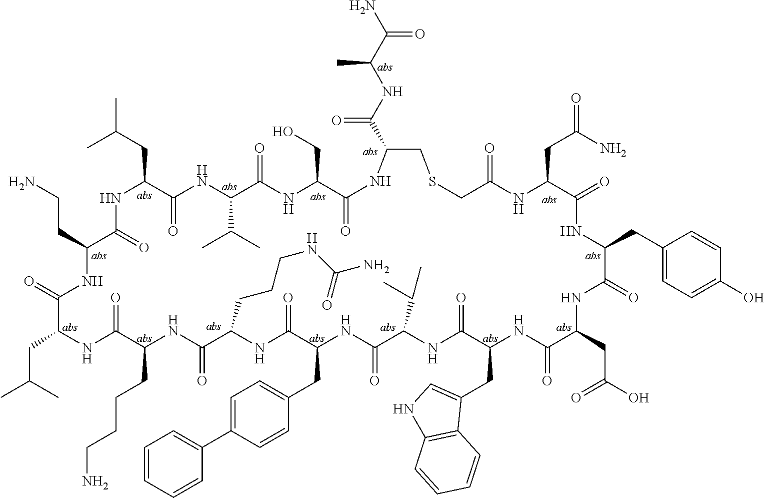 [SMILES: CC(C)C[C@@H]1NC(=O)[C@H](CCN)NC(=O)[C@@H](CC(C)C)NC(=O)[C@H](CCCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1110 was prepared on a 50 mol scale. The yield of the product was 6.1 mg, and its estimated purity by LCMS analysis was 91%. Analysis condition B: Retention time=1.31 min; ESI-MS(+) m/z [M+2H]²⁺: 965.9.

### Preparation of Compound 1111

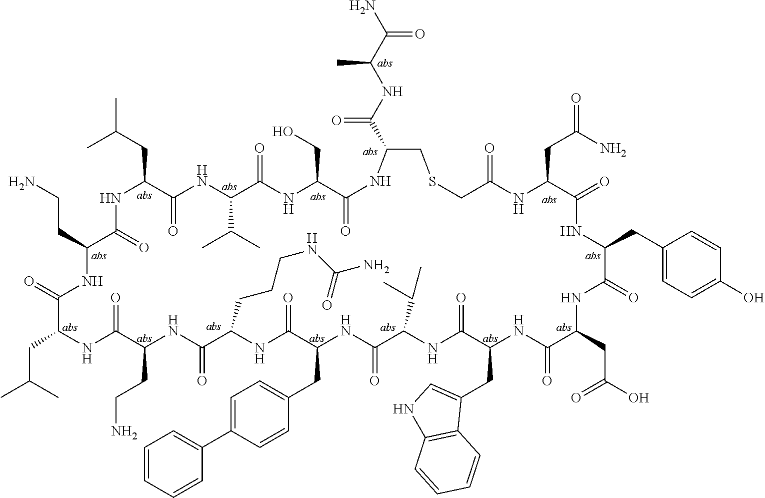 [SMILES: CC(C)C[C@@H]1NC(=O)[C@H](CCN)NC(=O)[C@@H](CC(C)C)NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1111 was prepared on a 50 μmol scale. The yield of the product was 7.5 mg, and its estimated purity by LCMS analysis was 95%. Analysis condition B: Retention time=1.28 min; ESI-MS(+) m/z [M+2H]²⁺: 952.1.

### Preparation of Compound 1112

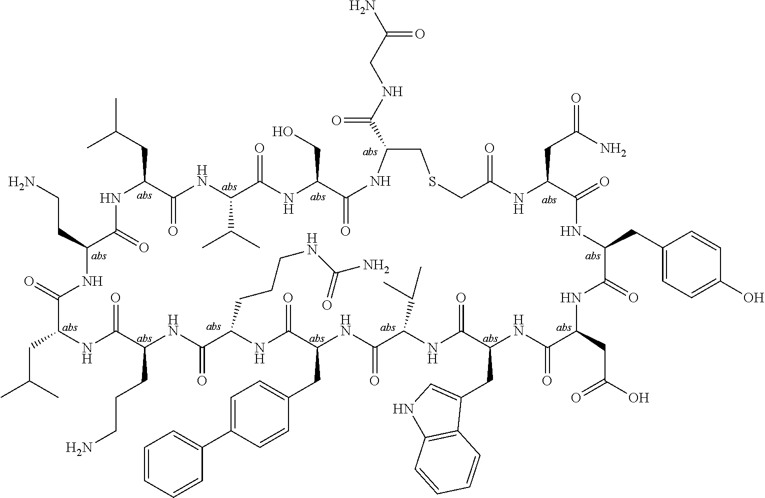 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1112 was prepared on a 50 μmol scale. The yield of the product was 5.8 mg, and its estimated purity by LCMS analysis was 91.1%. Analysis condition B: Retention time=1.43 min; ESI-MS(+) m/z [M+H]⁺: 1903.2.

### Preparation of Compound 1113

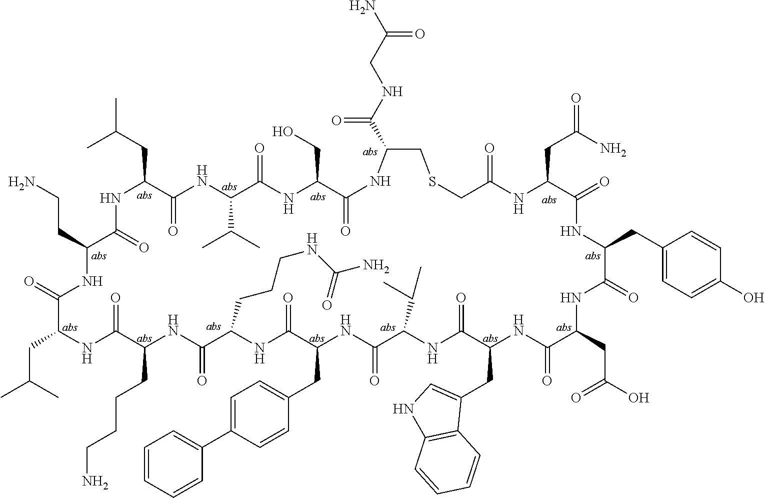 [SMILES: CC(C)C[C@@H]1NC(=O)[C@H](CCN)NC(=O)[C@@H](CC(C)C)NC(=O)[C@H](CCCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1113 was prepared on a 50 mol scale. The yield of the product was 7 mg, and its estimated purity by LCMS analysis was 94.7%. Analysis condition A: Retention time=1.35 min; ESI-MS(+) m/z [M+2H]²⁺: 959.1.

### Preparation of Compound 1114

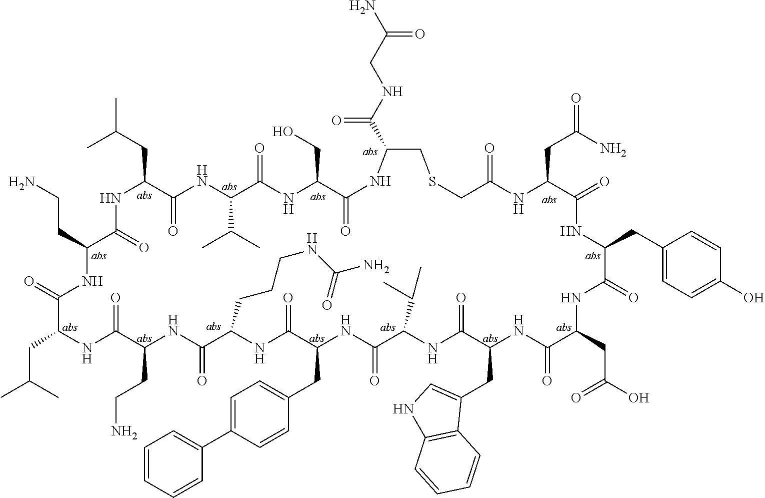 [SMILES: CC(C)C[C@@H]1NC(=O)[C@H](CCN)NC(=O)[C@@H](CC(C)C)NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1114 was prepared on a 50 mol scale. The yield of the product was 11.5 mg, and its estimated purity by LCMS analysis was 91.7%. Analysis condition B: Retention time=1.3 min; ESI-MS(+) m/z [M+2H]²⁺: 945.1.

### Preparation of Compound 1115

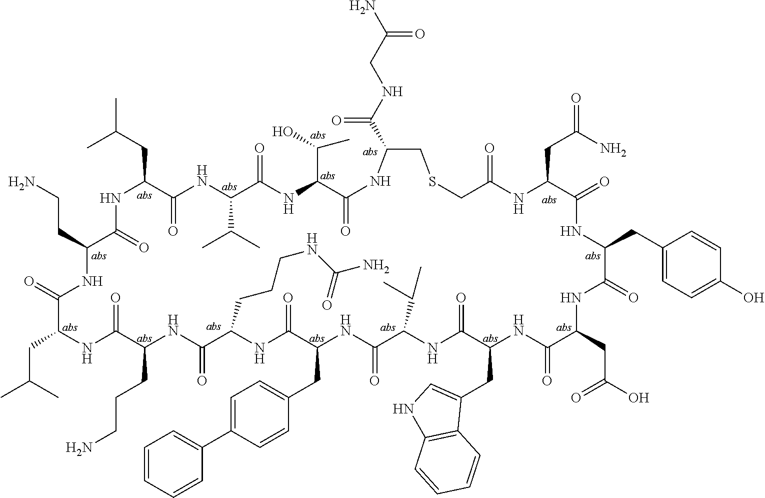 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1115 was prepared on a 50 mol scale. The yield of the product was 10 mg, and its estimated purity by LCMS analysis was 96.5%. Analysis condition B: Retention time=1.47 min; ESI-MS(+) m/z [M+H]⁺: 1915.8.

### Preparation of Compound 1116

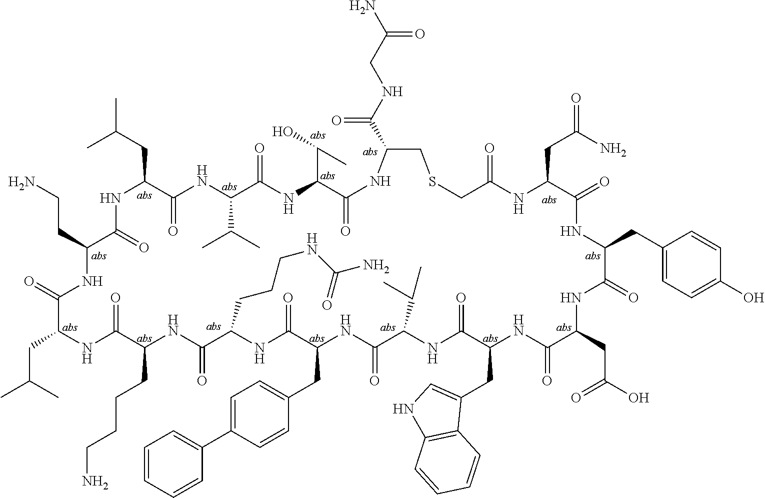 [SMILES: CC(C)C[C@@H]1NC(=O)[C@H](CCN)NC(=O)[C@@H](CC(C)C)NC(=O)[C@H](CCCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1116 was prepared on a 50 μmol scale. The yield of the product was 15.1 mg, and its estimated purity by LCMS analysis was 94%. Analysis condition B: Retention time=1.37 min; ESI-MS(+) m/z [M+2H]²⁺: 966.2.

### Preparation of Compound 1117

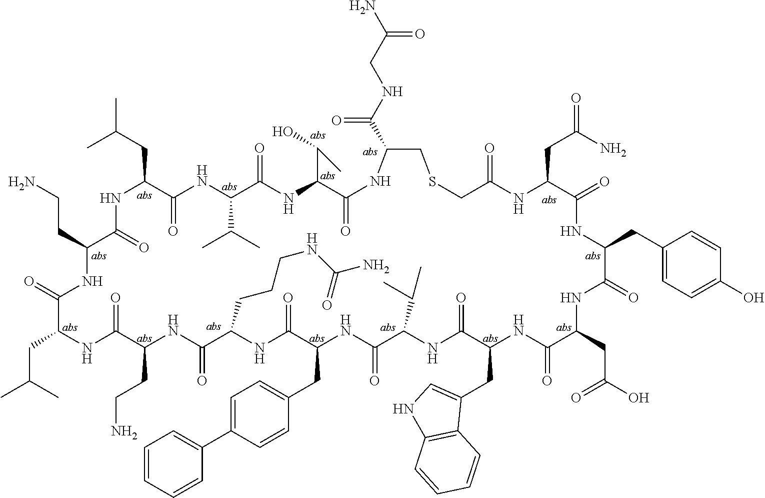 [SMILES: CC(C)C[C@@H]1NC(=O)[C@H](CCN)NC(=O)[C@@H](CC(C)C)NC(=O)[C@H](CCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1117 was prepared on a 50 μmol scale. The yield of the product was 8.5 mg, and its estimated purity by LCMS analysis was 96.9%. Analysis condition A: Retention time=1.5 min; ESI-MS(+) m/z [M+2H]²⁺: 952.2.

### Preparation of Compound 1118

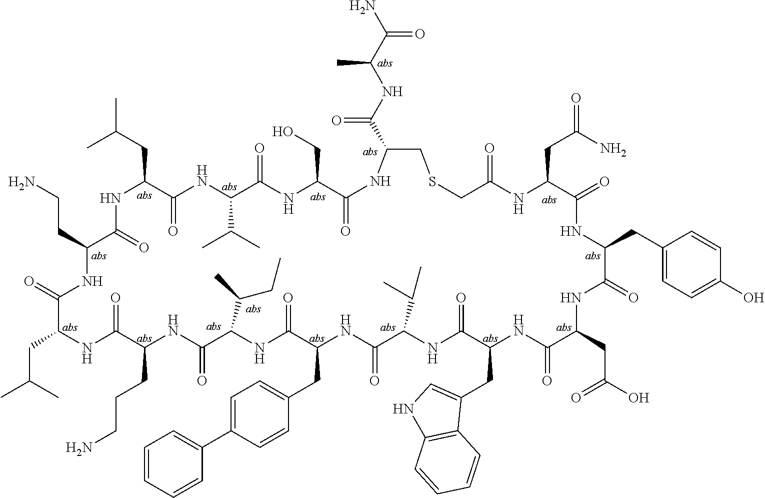 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1118 was prepared on a 50 μmol scale. The yield of the product was 6.2 mg, and its estimated purity by LCMS analysis was 86.3%. Analysis condition A: Retention time=1.49 min; ESI-MS(+) m/z [M+2H]²⁺: 936.8.

### Preparation of Compound 1119

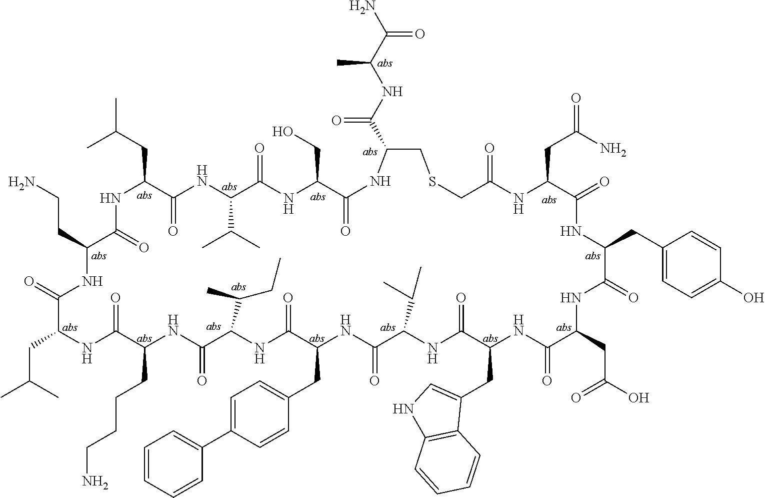 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCCN)NC1=O]

Compound 1119 was prepared on a 50 mol scale. The yield of the product was 2.6 mg, and its estimated purity by LCMS analysis was 91.4%. Analysis condition B: Retention time=1.36 min; ESI-MS(+) m/z [M+2H]²⁺: 943.8.

### Preparation of Compound 1120

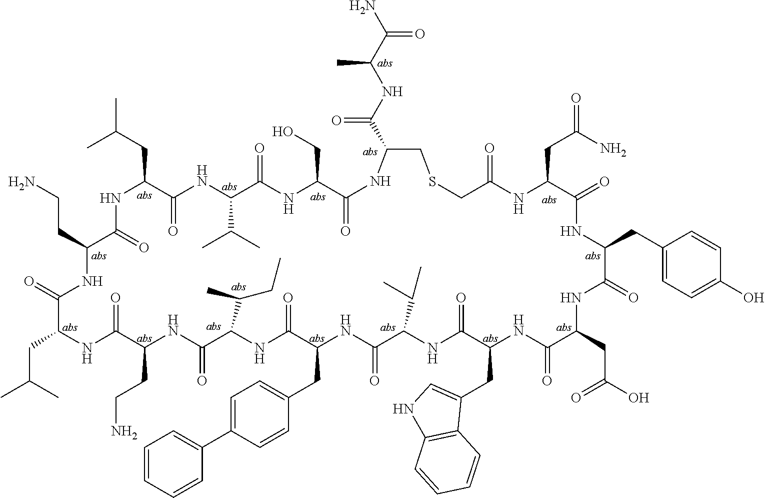 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 1120 was prepared on a 50 mol scale. The yield of the product was 5.6 mg, and its estimated purity by LCMS analysis was 98.4%. Analysis condition A: Retention time=1.64 min; ESI-MS(+) m/z [M+H]⁺: 1858.3.

### Preparation of Compound 1121

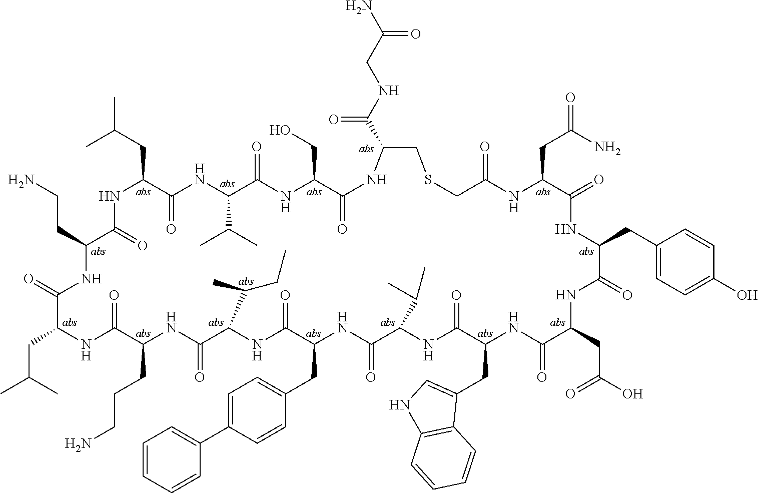 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1121 was prepared on a 50 mol scale. The yield of the product was 11.6 mg, and its estimated purity by LCMS analysis was 91.9%. Analysis condition B: Retention time=1.49 min; ESI-MS(+) m/z [M+2H]²⁺: 930.1.

### Preparation of Compound 1122

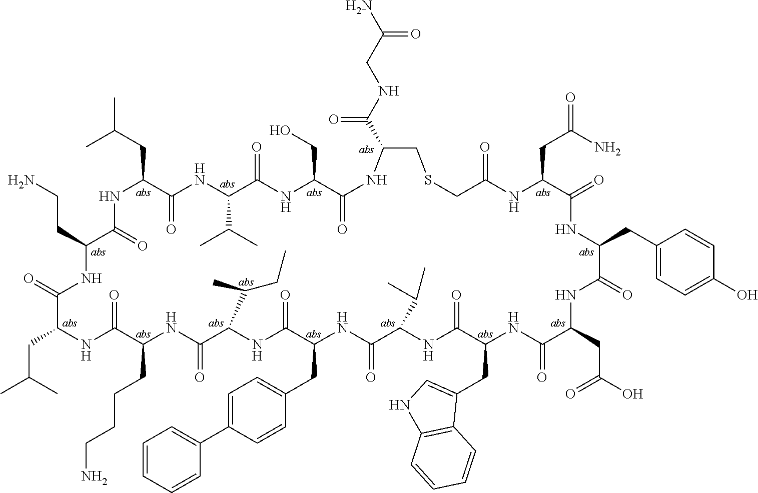 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCCN)NC1=O]

Compound 1122 was prepared on a 50 μmol scale. The yield of the product was 8 mg, and its estimated purity by LCMS analysis was 92.6%. Analysis condition A: Retention time=1.63 min; ESI-MS(+) m/z [M+2H]²⁺: 937.

### Preparation of Compound 1123

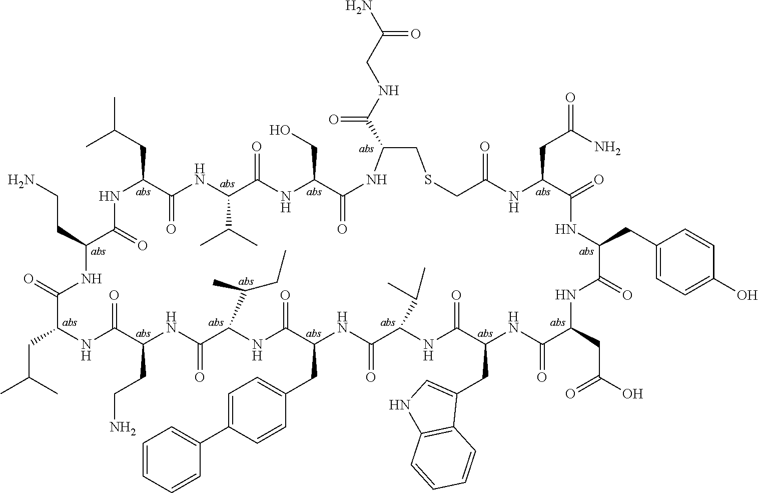 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 1123 was prepared on a 50 mol scale. The yield of the product was 8.4 mg, and its estimated purity by LCMS analysis was 91%. Analysis condition A: Retention time=1.48 min; ESI-MS(+) m/z [M+2H]²⁺: 922.9.

### Preparation of Compound 1124

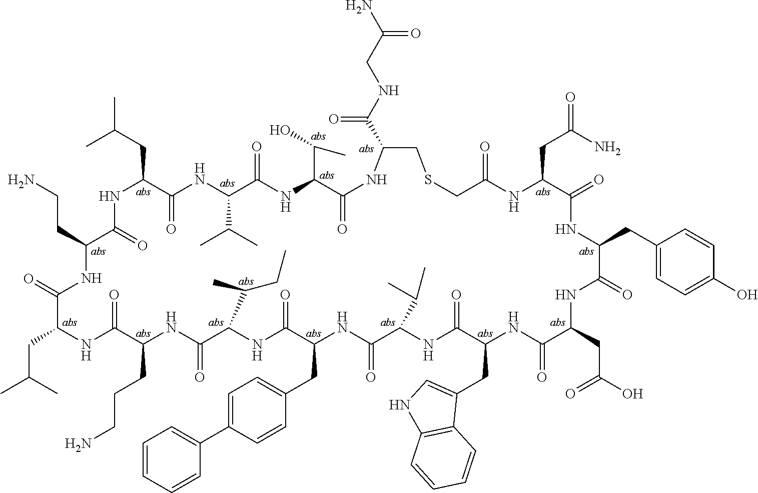 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1124 was prepared on a 50 μmol scale. The yield of the product was 11.6 mg, and its estimated purity by LCMS analysis was 95.8%. Analysis condition A: Retention time=1.67 min; ESI-MS(+) m/z [M+H]⁺: 1872.2.

### Preparation of Compound 1125

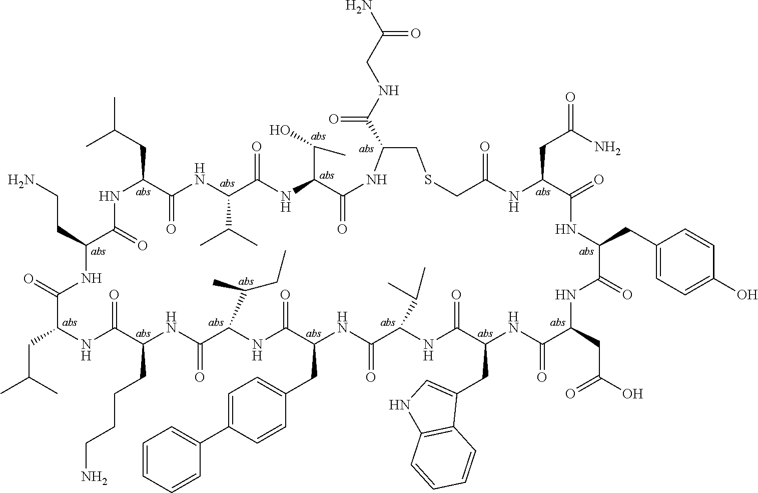 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCCN)NC1=O]

Compound 1125 was prepared on a 50 mol scale. The yield of the product was 11.2 mg, and its estimated purity by LCMS analysis was 96.3%. Analysis condition B: Retention time=1.7 min: ESI-MS(+) m/z [M+H]⁺: 1891.

### Preparation of Compound 1126

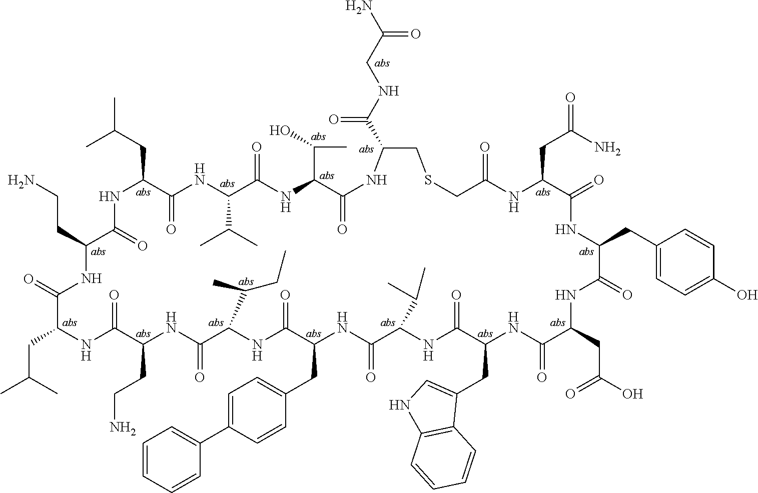 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 1126 was prepared on a 50 μmol scale. The yield of the product was 9 mg, and its estimated purity by LCMS analysis was 80%. Analysis condition B: Retention time=1.51 min; ESI-MS(+) m/z [M+2H]²⁺: 929.9.

### Preparation of Compound 1127

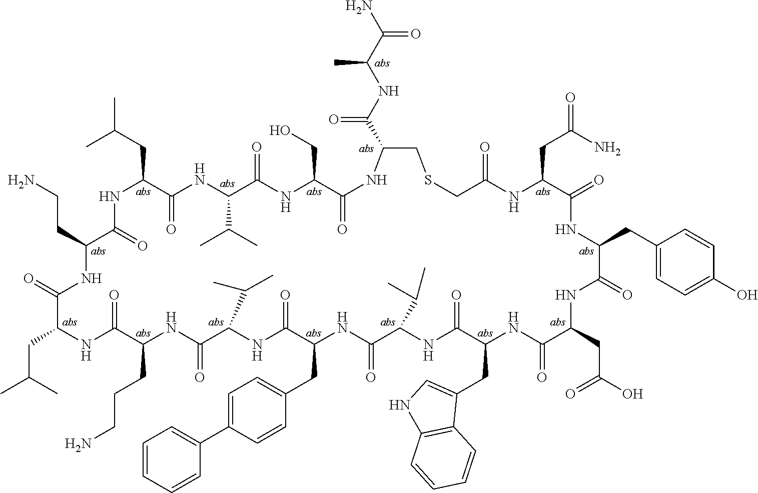 [SMILES: CC(C)C[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1127 was prepared on a 50 μmol scale. The yield of the product was 4.2 mg, and its estimated purity by LCMS analysis was 87.1%. Analysis condition B: Retention time=1.46 min; ESI-MS(+) m/z [M+2H]²⁺: 930.2.

### Preparation of Compound 1128

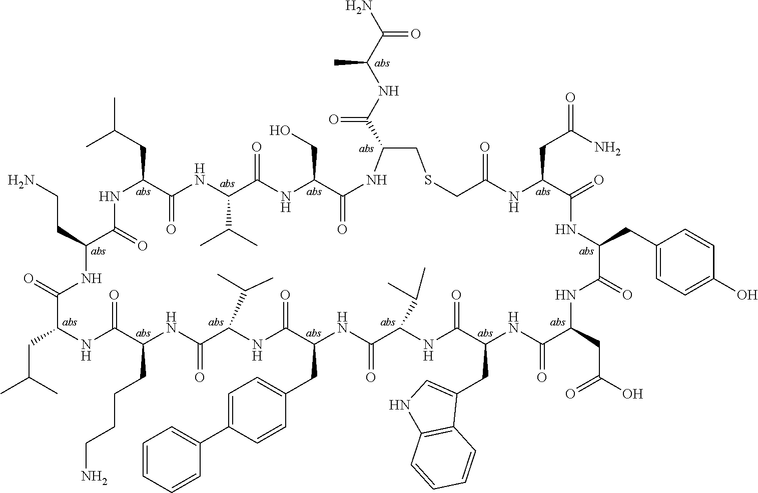 [SMILES: CC(C)C[C@@H]1NC(=O)[C@H](CCN)NC(=O)[C@@H](CC(C)C)NC(=O)[C@H](CCCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1128 was prepared on a 50 mol scale. The yield of the product was 3.5 mg, and its estimated purity by LCMS analysis was 90.7%. Analysis condition B: Retention time=1.48 min; ESI-MS(+) m/z [M+2H]²⁺: 937.2.

### Preparation of Compound 1129

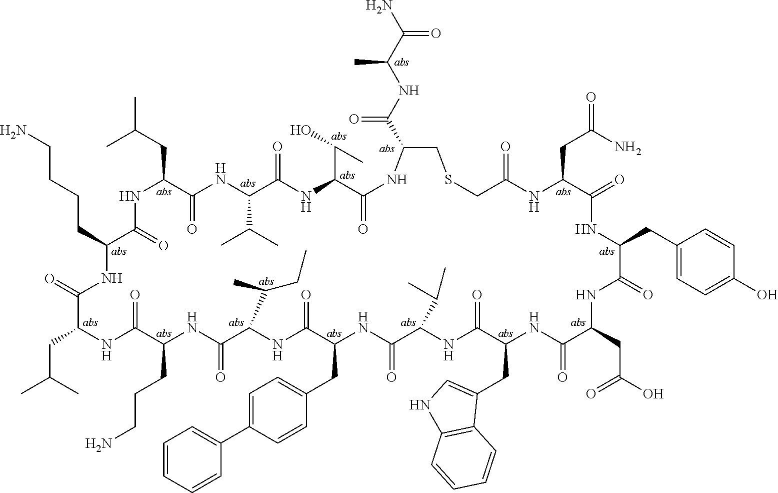 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCCN)NC(=O)[C@@H](CC(C)C)NC(=O)[C@H](CCCN)NC1=O]

Compound 1129 was prepared on a 50 mol scale. The yield of the product was 2.3 mg, and its estimated purity by LCMS analysis was 100%. Analysis condition A: Retention time=1.68 min; ESI-MS(+) m/z [M+2H]²⁺: 958.1.

### Preparation of Compound 1130

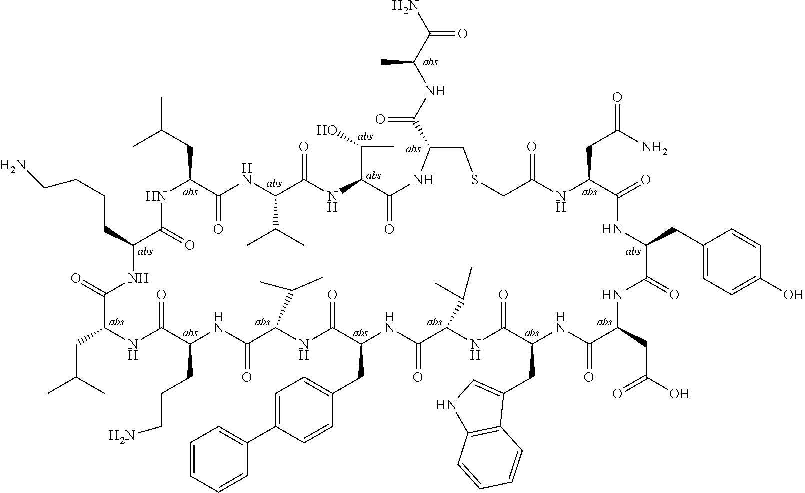 [SMILES: CC(C)C[C@@H]1NC(=O)[C@H](CCCCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1130 was prepared on a 50 mol scale. The yield of the product was 3.1 mg, and its estimated purity by LCMS analysis was 93.5%. Analysis condition B: Retention time=1.36 min; ESI-MS(+) m/z [M+2H]²⁺: 950.9.

### Preparation of Compound 1131

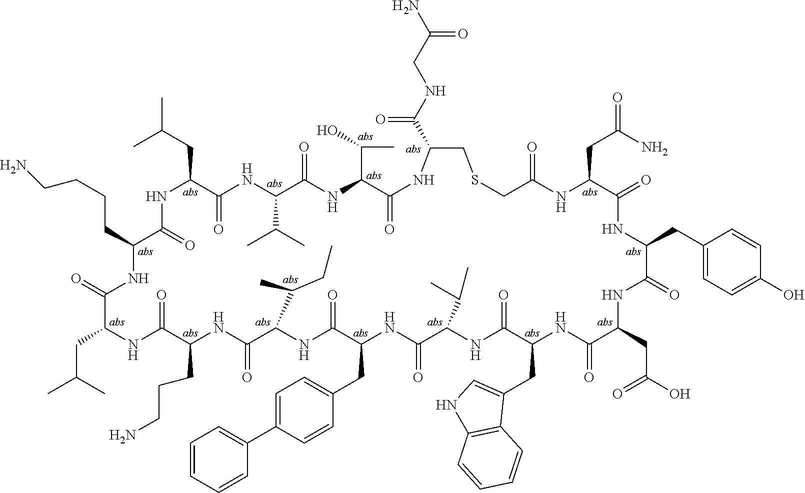 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC(=O)[C@H](CC(C)C)NC(=O)[C@H](CCCCN)NC(=O)[C@@H](CC(C)C)NC(=O)[C@H](CCCN)NC1=O]

Compound 1131 was prepared on a 50 μmol scale. The yield of the product was 6.4 mg, and its estimated purity by LCMS analysis was 96.1%. Analysis condition A: Retention time=1.67 min; ESI-MS(+) m/z [M+H]⁺: 1901.2.

### Preparation of Compound 1132

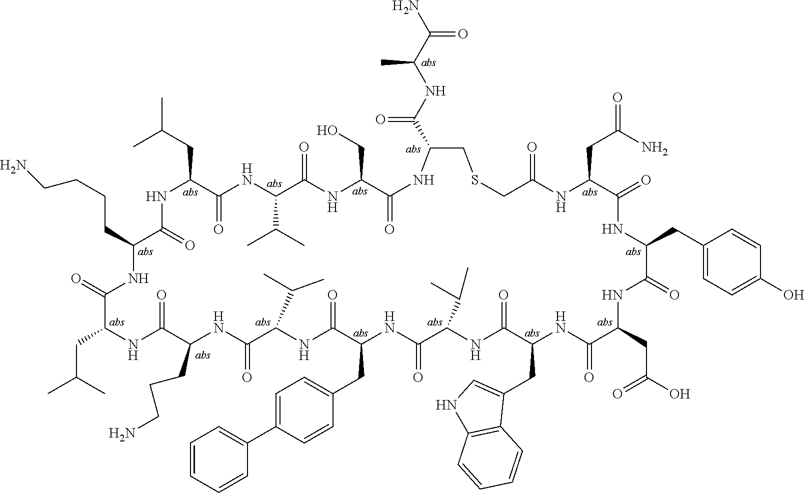 [SMILES: CC(C)C[C@@H]1NC(=O)[C@H](CCCCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1132 was prepared on a 50 μmol scale. The yield of the product was 1.5 mg, and its estimated purity by LCMS analysis was 86.5%. Analysis condition A: Retention time=1.44 min; ESI-MS(+) m/z [M+2H]²⁺: 944.2.

### Preparation of Compound 1133

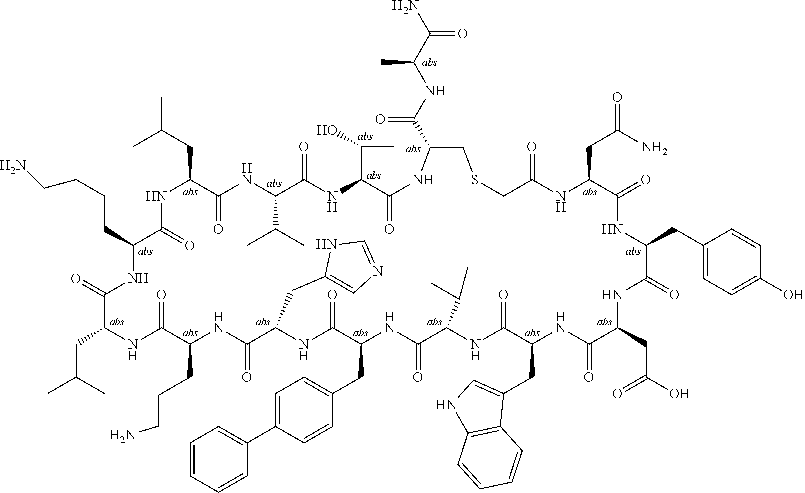 [SMILES: CC(C)C[C@@H]1NC(=O)[C@H](CCCCN)NC(=O)[C@@H](CC(C)C)NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1133 was prepared on a 50 μmol scale. The yield of the product was 14.8 mg, and its estimated purity by LCMS analysis was 94.3%. Analysis condition B: Retention time=1.26 min; ESI-MS(+) m/z [M+2H]²⁺: 970.2.

### Preparation of Compound 1134

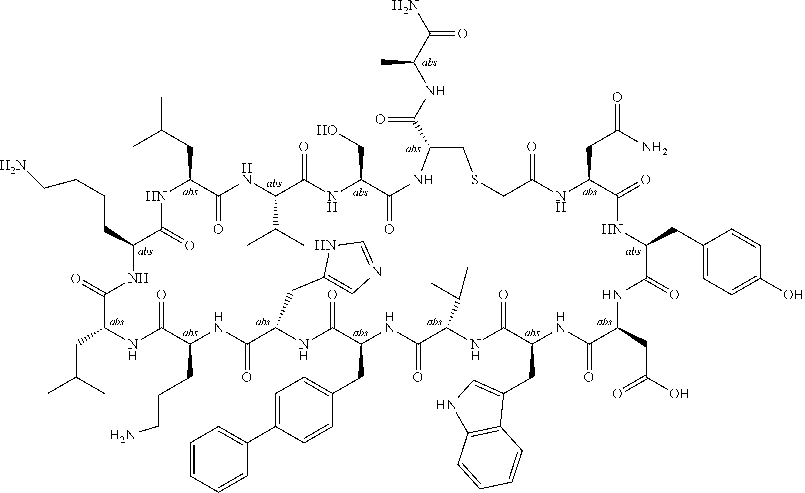 [SMILES: CC(C)C[C@@H]1NC(=O)[C@H](CCCCN)NC(=O)[C@@H](CC(C)C)NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1134 was prepared on a 50 μmol scale. The yield of the product was 2.5 mg, and its estimated purity by LCMS analysis was 93.3%. Analysis condition A: Retention time=1.48 min; ESI-MS(+) m/z [M+2H]²⁺: 963.2.

### Preparation of Compound 1135

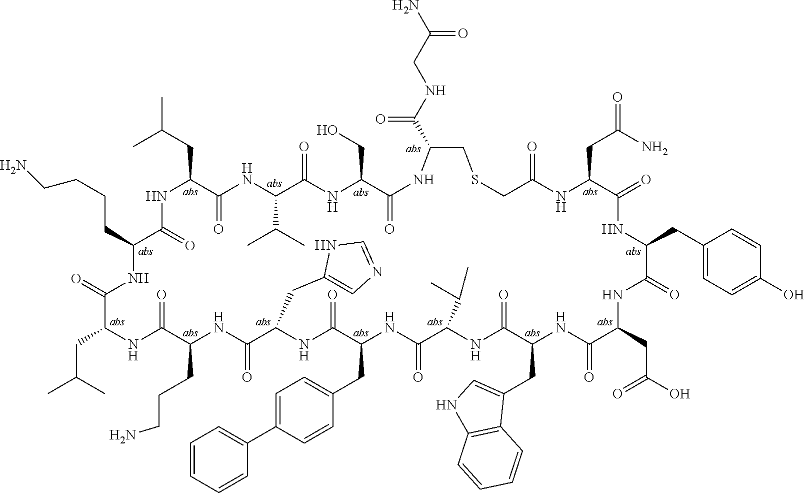 [SMILES: CC(C)C[C@@H]1NC(=O)[C@H](CCCCN)NC(=O)[C@@H](CC(C)C)NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1135 was prepared on a 50 μmol scale. The yield of the product was 10.8 mg, and its estimated purity by LCMS analysis was 94.7%. Analysis condition B: Retention time=1.24 min; ESI-MS(+) m/z [M+2H]²⁺: 956.

### Preparation of Compound 1136

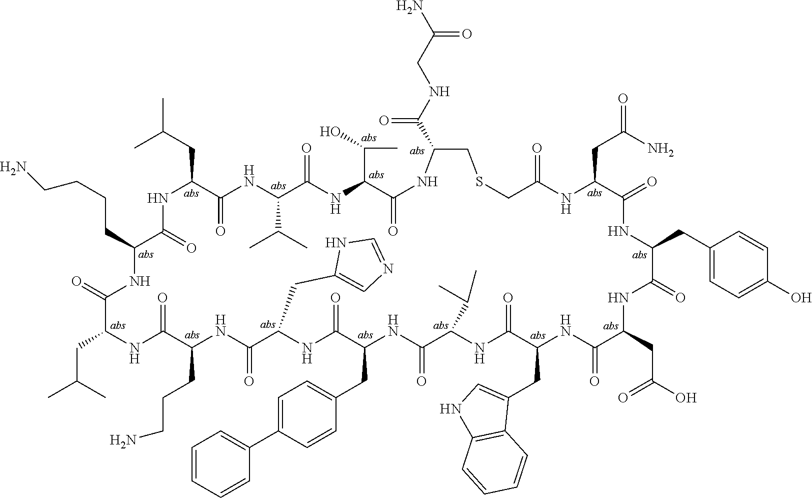 [SMILES: CC(C)C[C@@H]1NC(=O)[C@H](CCCCN)NC(=O)[C@@H](CC(C)C)NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1136 was prepared on a 50 μmol scale. The yield of the product was 9.4 mg, and its estimated purity by LCMS analysis was 97.9%. Analysis condition B: Retention time=1.38 min; ESI-MS(+) m/z [M+2H]²⁺: 963.3.

### Preparation of Compound 1137

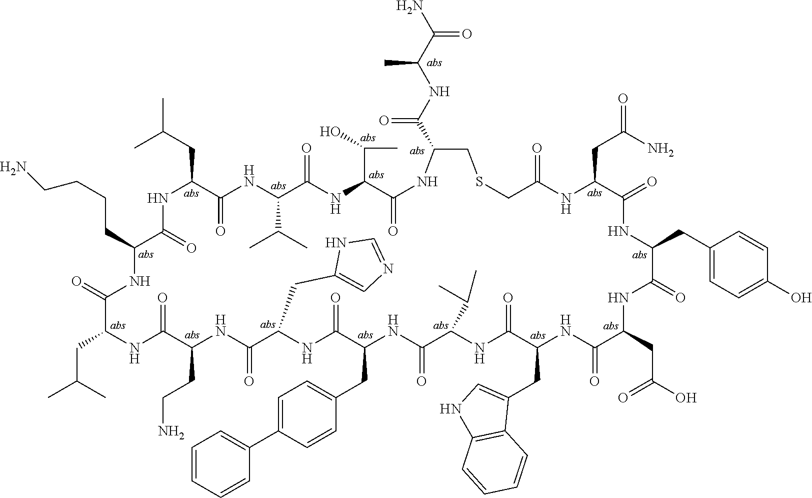 [SMILES: CC(C)C[C@@H]1NC(=O)[C@H](CCCCN)NC(=O)[C@@H](CC(C)C)NC(=O)[C@H](CCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1137 was prepared on a 50 μmol scale. The yield of the product was 12.8 mg, and its estimated purity by LCMS analysis was 94.1%. Analysis condition B: Retention time=1.38 min; ESI-MS(+) m/z [M+H]⁺: 1925.3.

### Preparation of Compound 1138

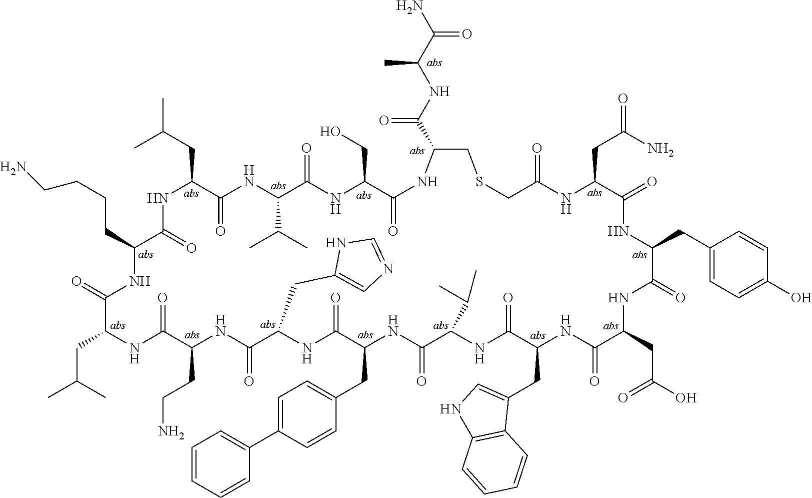 [SMILES: CC(C)C[C@@H]1NC(=O)[C@H](CCCCN)NC(=O)[C@@H](CC(C)C)NC(=O)[C@H](CCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC1=O]

Compound 1138 was prepared on a 50 μmol scale. The yield of the product was 8.2 mg, and its estimated purity by LCMS analysis was 97.8%. Analysis condition B: Retention time=1.36 min; ESI-MS(+) m/z [M+H]⁺: 1911.

### Preparation of Compound 1139

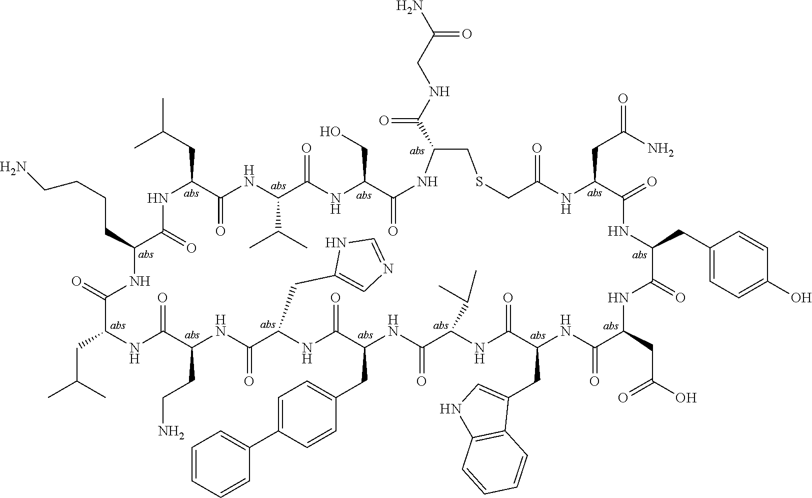 [SMILES: CC(C)C[C@@H]1NC(=O)[C@H](CCCCN)NC(=O)[C@@H](CC(C)C)NC(=O)[C@H](CCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H](CO)NC(=O)[C@H](C(C)C)NC1=O]

Compound 1139 was prepared on a 50 μmol scale. The yield of the product was 17.5 mg, and its estimated purity by LCMS analysis was 97.7%. Analysis condition B: Retention time=1.36 min; ESI-MS(+) m/z [M+H]⁺: 1896.9.

### Preparation of Compound 1140

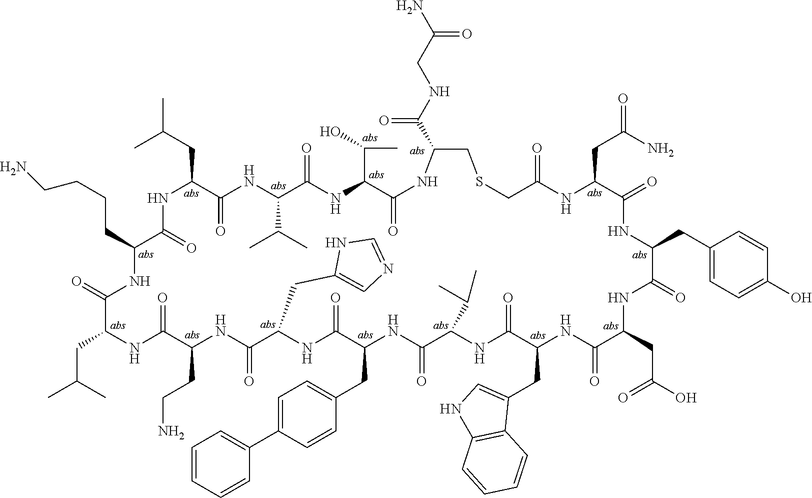 [SMILES: CC(C)C[C@@H]1NC(=O)[C@H](CCCCN)NC(=O)[C@@H](CC(C)C)NC(=O)[C@H](CCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)NCC(N)=O)NC(=O)[C@H]([C@@H](C)O)NC(=O)[C@H](C(C)C)NC1=O]

Compound 1140 was prepared on a 50 μmol scale. The yield of the product was 12.9 mg, and its estimated purity by LCMS analysis was 96.7%. Analysis condition B: Retention time=1.27 min; ESI-MS(+) m/z [M+2H]²⁺: 956.1.

### Preparation of Compound 1141

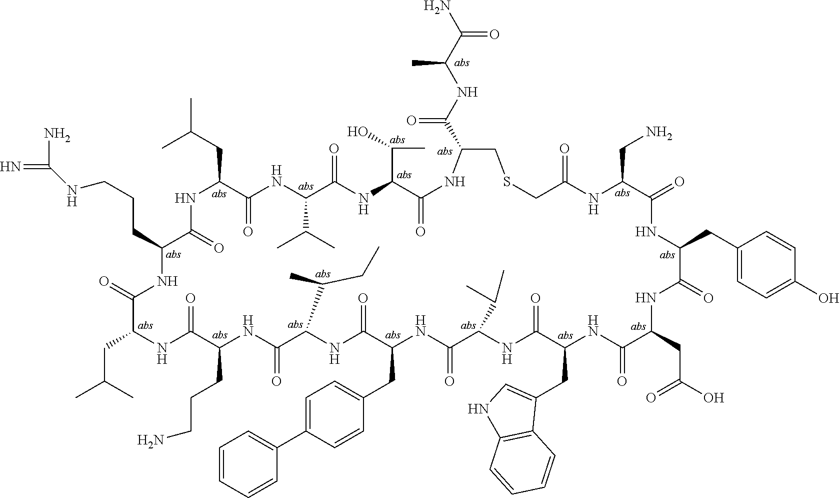 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@@H](CC(C)C)NC(=O)[C@H](CCCN)NC1=O]

Compound 1141 was prepared on a 50 mol scale. The yield of the product was 5 mg, and its estimated purity by LCMS analysis was 88.9%. Analysis condition B: Retention time=1.44 min; ESI-MS(+) m/z [M+H]⁺: 1913.6.

### Preparation of Compound 1142

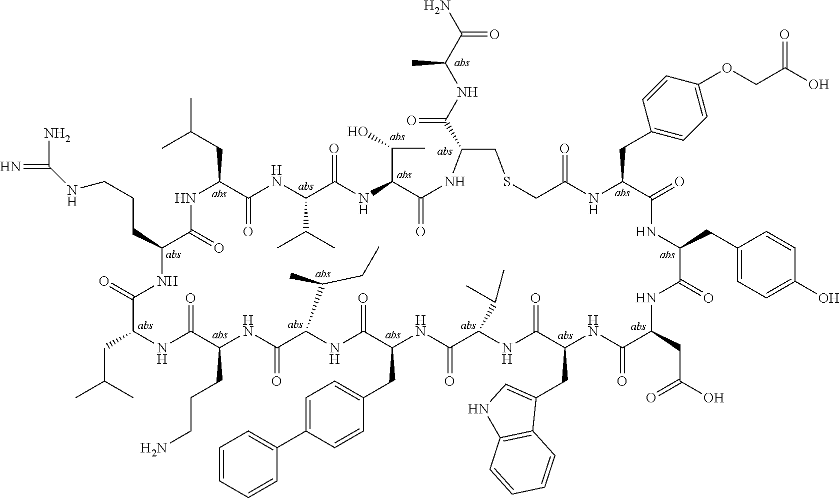 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@@H](CC(C)C)NC(=O)[C@H](CCCN)NC1=O]

Compound 1142 was prepared on a 50 μmol scale. The yield of the product was 10.4 mg, and its estimated purity by LCMS analysis was 86.1%. Analysis condition A: Retention time=1.77 min; ESI-MS(+) m/z [M+2H]²⁺: 1025.

### Preparation of Compound 1143

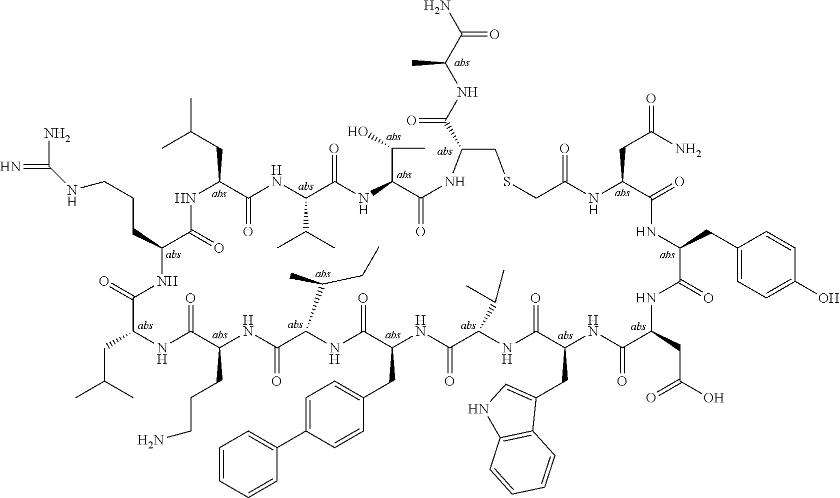 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@@H](CC(C)C)NC(=O)[C@H](CCCN)NC1=O]

Compound 1143 was prepared on a 50 μmol scale. The yield of the product was 2.1 mg, and its estimated purity by LCMS analysis was 100%. Analysis condition A: Retention time=1.56 min; ESI-MS(+) m/z [M+2H]²⁺: 971.9.

### Preparation of Compound 1144

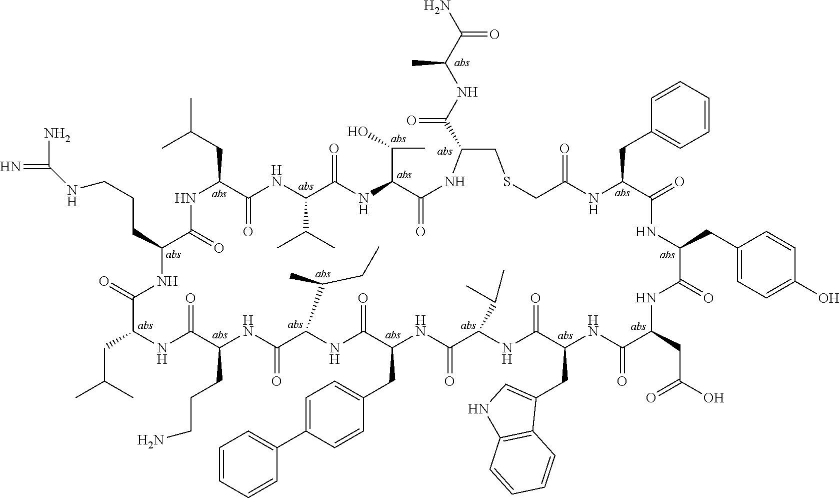 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCCNC(=N)N)NC(=O)[C@@H](CC(C)C)NC(=O)[C@H](CCCN)NC1=O]

Compound 1144 was prepared on a 50 μmol scale. The yield of the product was 6.5 mg, and its estimated purity by LCMS analysis was 100%. Analysis condition A: Retention time=1.72 min; ESI-MS(+) m/z [M+2H]²⁺: 988.

### Preparation of Compound 1145

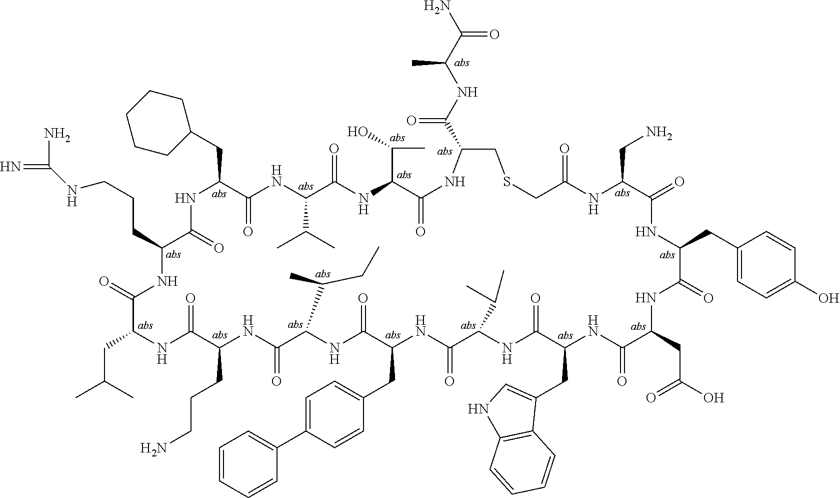 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC(=O)[C@@H](CC(C)C)NC(=O)[C@H](CCCN)NC1=O]

Compound 1145 was prepared on a 50 μmol scale. The yield of the product was 8 mg, and its estimated purity by LCMS analysis was 99.3%. Analysis condition B: Retention time=1.41 min; ESI-MS(+) m/z [M+3H]³⁺: 652.3.

### Preparation of Compound 1146

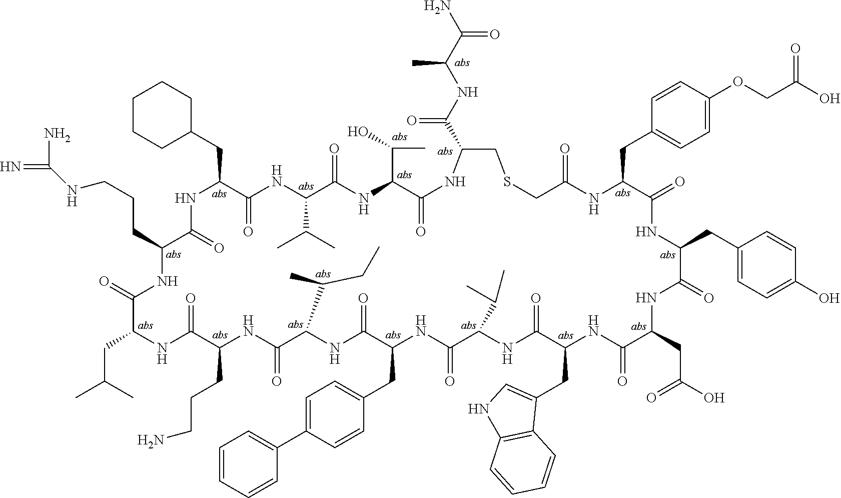 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC(=O)[C@@H](CC(C)C)NC(=O)[C@H](CCCN)NC1=O]

Compound 1146 was prepared on a 50 μmol scale. The yield of the product was 9.9 mg, and its estimated purity by LCMS analysis was 92.2%. Analysis condition A: Retention time=1.93 min; ESI-MS(+) m/z [M+2H]²⁺: 1045.2.

### Preparation of Compound 1147

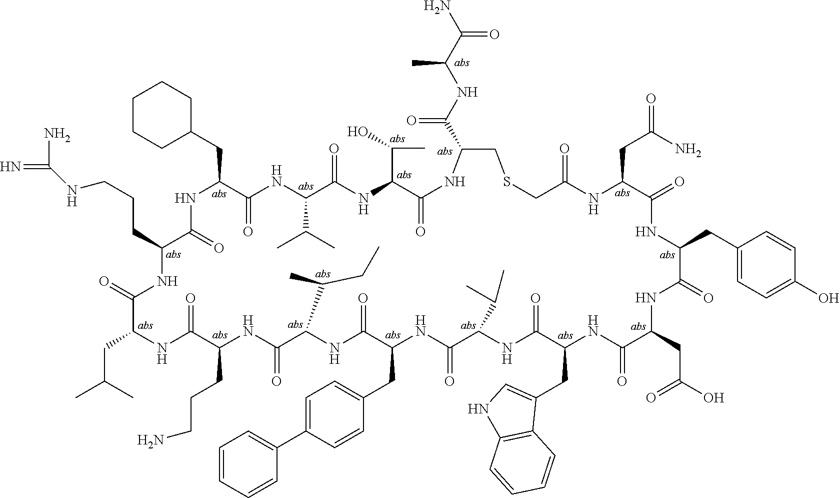 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC(=O)[C@@H](CC(C)C)NC(=O)[C@H](CCCN)NC1=O]

Compound 1147 was prepared on a 50 μmol scale. The yield of the product was 6.4 mg, and its estimated purity by LCMS analysis was 90.1%. Analysis condition A: Retention time=1.7, 1.75 min; ESI-MS(+) m/z [M+2H]²⁺: 992.1.

### Preparation of Compound 1148

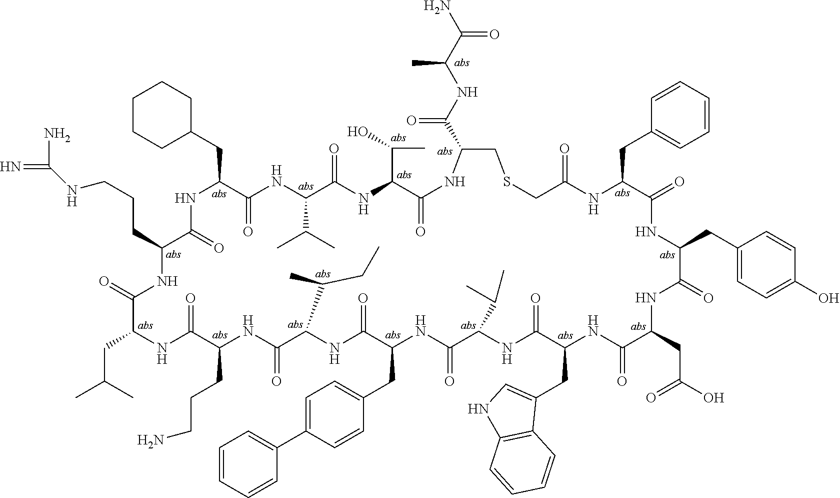 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC(=O)[C@@H](CC(C)C)NC(=O)[C@H](CCCN)NC1=O]

Compound 1148 was prepared on a 50 μmol scale. The yield of the product was 9.3 mg, and its estimated purity by LCMS analysis was 92.5%. Analysis condition A: Retention time=1.95 min; ESI-MS(+) m/z [M+2H]²⁺: 1008.1.

### Preparation of Compound 1149

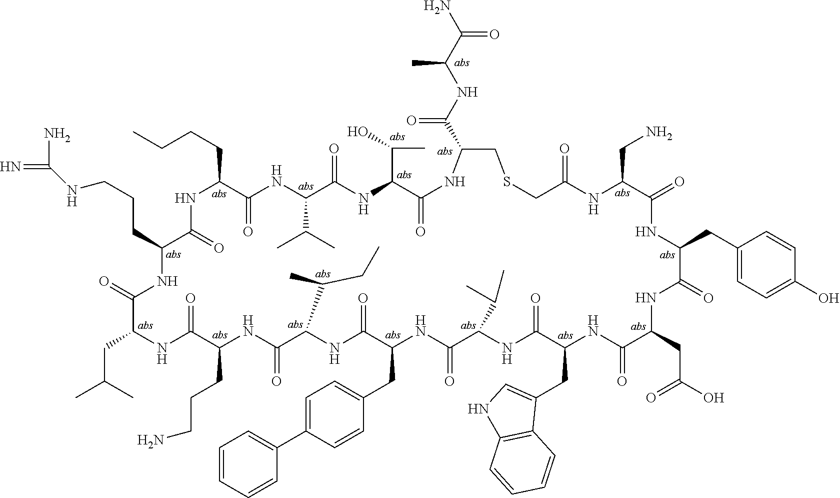 [SMILES: CCCC[C@@H]1NC(=O)[C@H](CCCNC(=N)N)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1149 was prepared on a 50 μmol scale. The yield of the product was 6.9 mg, and its estimated purity by LCMS analysis was 91.3%. Analysis condition B: Retention time=1.4, 1.47 min; ESI-MS(+) m/z [M+2H]²⁺: 957.92, 958.24.

### Preparation of Compound 1150

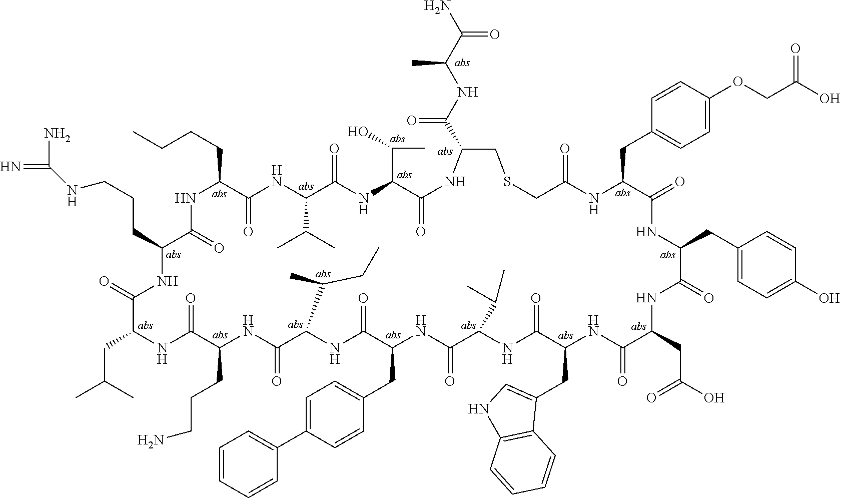 [SMILES: CCCC[C@@H]1NC(=O)[C@H](CCCNC(=N)N)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1150 was prepared on a 50 μmol scale. The yield of the product was 10.6 mg, and its estimated purity by LCMS analysis was 91.9%. Analysis condition A: Retention time=1.83 min; ESI-MS(+) m/z [M+2H]²⁺: 1025.2.

### Preparation of Compound 1151

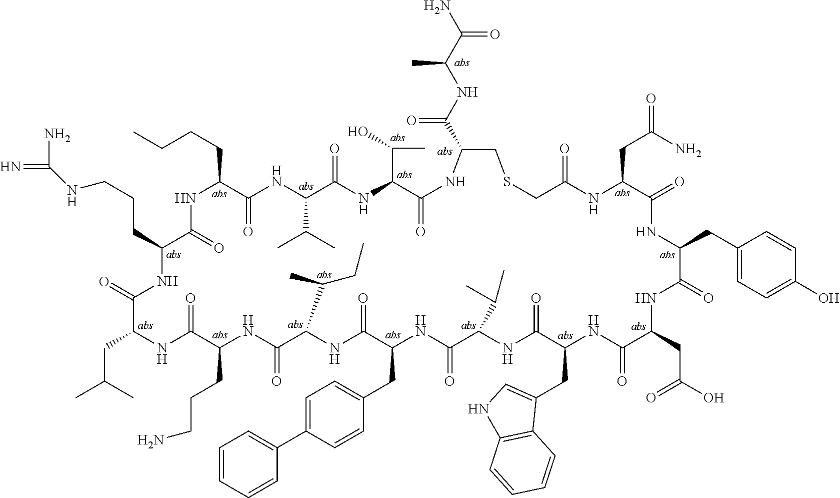 [SMILES: CCCC[C@@H]1NC(=O)[C@H](CCCNC(=N)N)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1151 was prepared on a 50 mol scale. The yield of the product was 3.3 mg, and its estimated purity by LCMS analysis was 85.5%. Analysis condition B: Retention time=1.57 min; ESI-MS(+) m/z [M+H]⁺: 1943.4.

### Preparation of Compound 1152

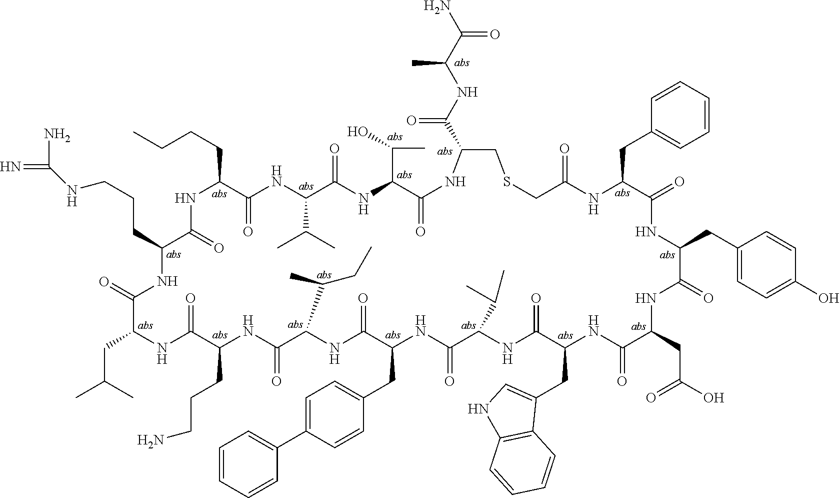 [SMILES: CCCC[C@@H]1NC(=O)[C@H](CCCNC(=N)N)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC1=O]

Compound 1152 was prepared on a 50 μmol scale. The yield of the product was 11.3 mg, and its estimated purity by LCMS analysis was 93.4%. Analysis condition A: Retention time=1.87 min; ESI-MS(+) m/z [M+2H]²⁺: 988.1.

### Preparation of Compound 1153

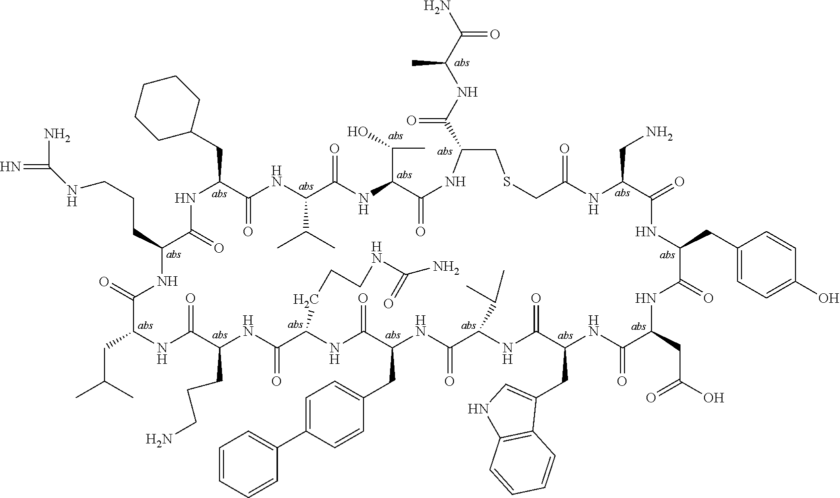 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC1=O]

Compound 1153 was prepared on a 50 μmol scale. The yield of the product was 14.3 mg, and its estimated purity by LCMS analysis was 99.4%. Analysis condition B: Retention time=1.37 min; ESI-MS(+) m/z [M+2H]²⁺: 1000.2.

### Preparation of Compound 1154

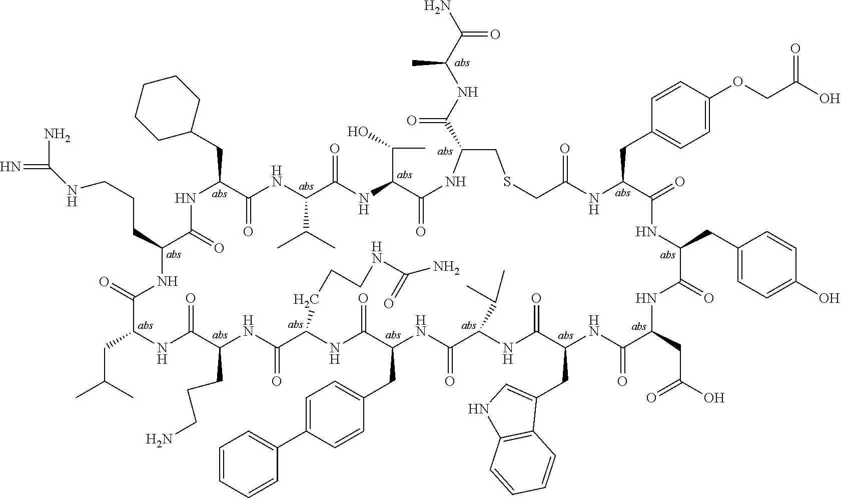 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC1=O]

Compound 1154 was prepared on a 50 μmol scale. The yield of the product was 12.2 mg, and its estimated purity by LCMS analysis was 100%. Analysis condition A: Retention time=1.62 min; ESI-MS(+) m/z [M+2H]²⁺: 1067.

### Preparation of Compound 1155

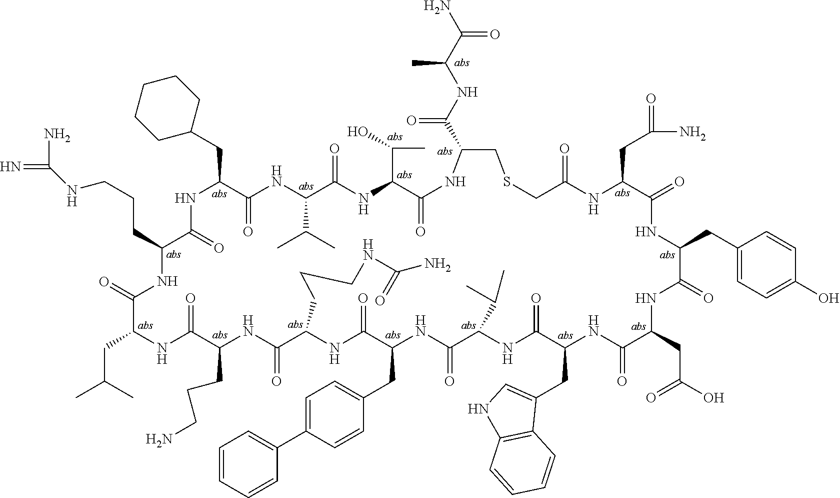 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC1=O]

Compound 1155 was prepared on a 50 μmol scale. The yield of the product was 12.8 mg, and its estimated purity by LCMS analysis was 96.6%. Analysis condition B: Retention time=1.55 min; ESI-MS(+) m/z [M+2H]²⁺: 1014.

### Preparation of Compound 1156

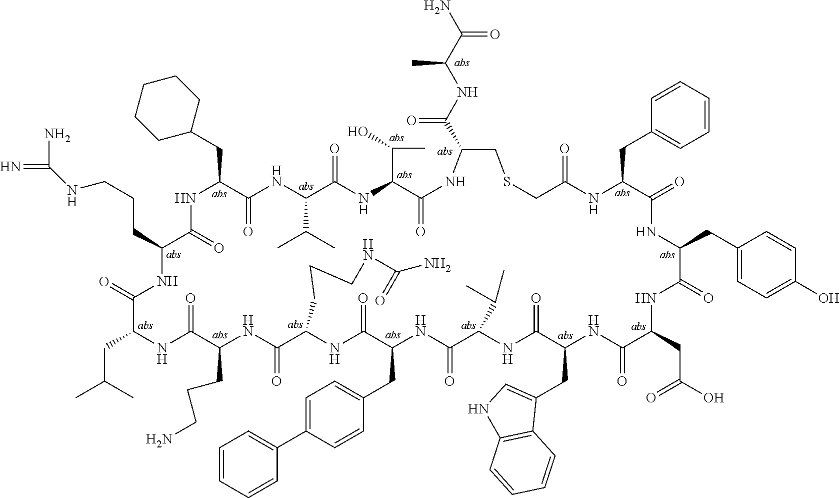 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCCNC(=N)N)NC1=O]

Compound 1156 was prepared on a 50 mol scale. The yield of the product was 32.1 mg, and its estimated purity by LCMS analysis was 84.8%. Analysis condition A: Retention time=1.65 min; ESI-MS(+) m/z [M+2H]²⁺: 1030.2.

### Preparation of Compound 1157

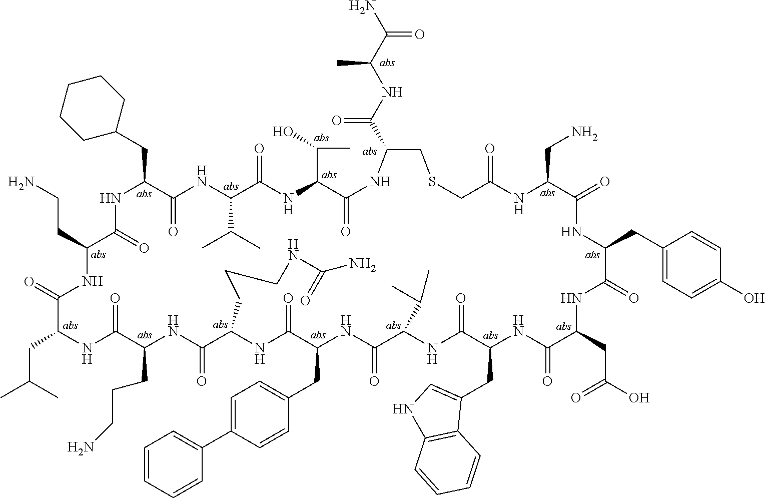 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1157 was prepared on a 50 mol scale. The yield of the product was 3.9 mg, and its estimated purity by LCMS analysis was 95.2%. Analysis condition B: Retention time=1.38 min; ESI-MS(+) m/z [M+2H]²⁺: 972.4.

### Preparation of Compound 1158

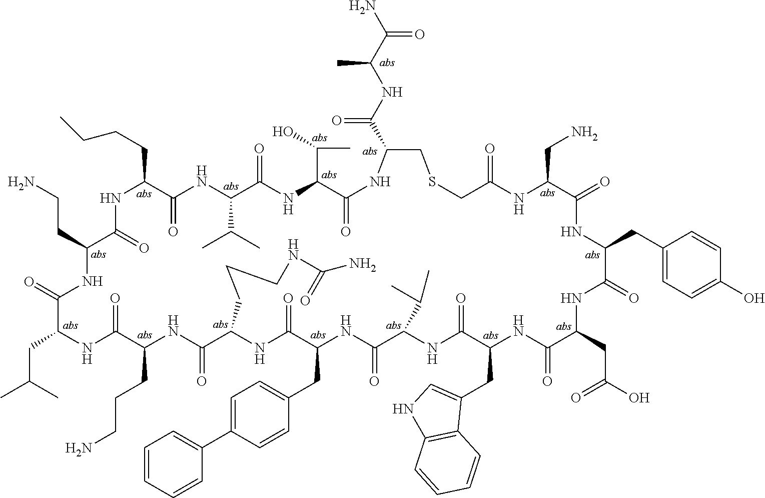 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1158 was prepared on a 50 mol scale. The yield of the product was 1.8 mg, and its estimated purity by LCMS analysis was 100%. Analysis condition A: Retention time=1.44 min; ESI-MS(+) m/z [M+H]⁺: 1902.

### Preparation of Compound 1159

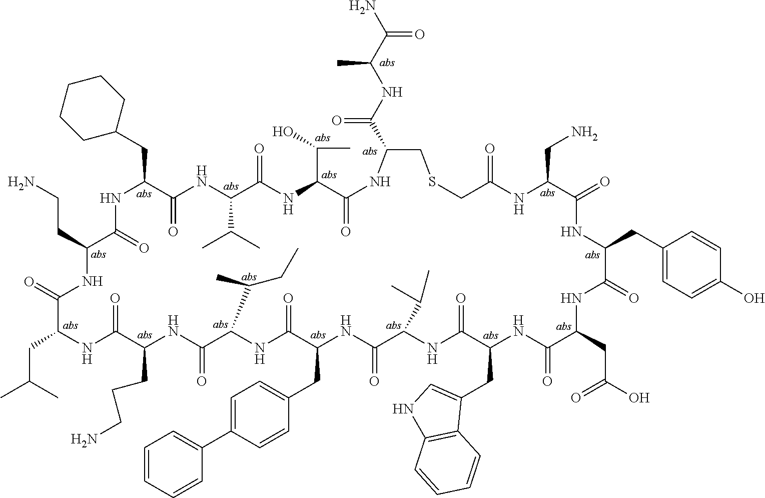 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1159 was prepared on a 50 μmol scale. The yield of the product was 6.7 mg, and its estimated purity by LCMS analysis was 92.2%. Analysis condition A: Retention time=1.53 min; ESI-MS(+) m/z [M+2H]²⁺: 950.

### Preparation of Compound 1160

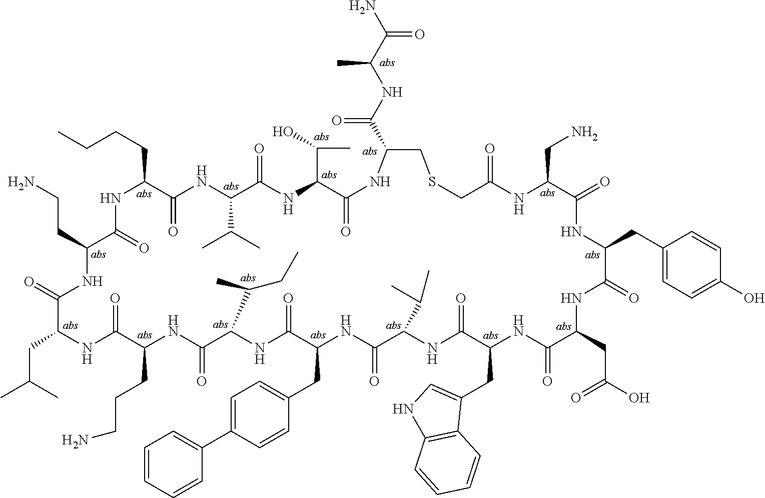 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1160 was prepared on a 50 mol scale. The yield of the product was 3.4 mg, and its estimated purity by LCMS analysis was 93.7%. Analysis condition A: Retention time=1.53 min; ESI-MS(+) m/z [M+2H]²⁺: 930.3.

### Preparation of Compound 1161

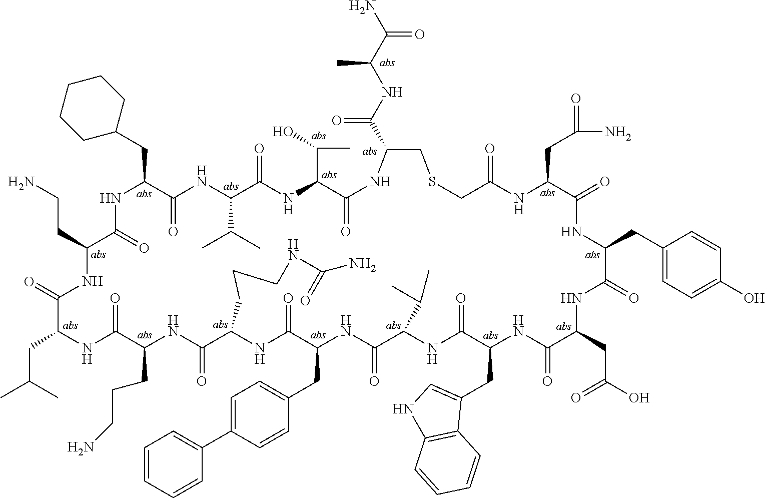 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1161 was prepared on a 50 μmol scale. The yield of the product was 2.3 mg, and its estimated purity by LCMS analysis was 95%. Analysis condition B: Retention time=1.45 min; ESI-MS(+) m/z [M+2H]²⁺: 986.2.

### Preparation of Compound 1162

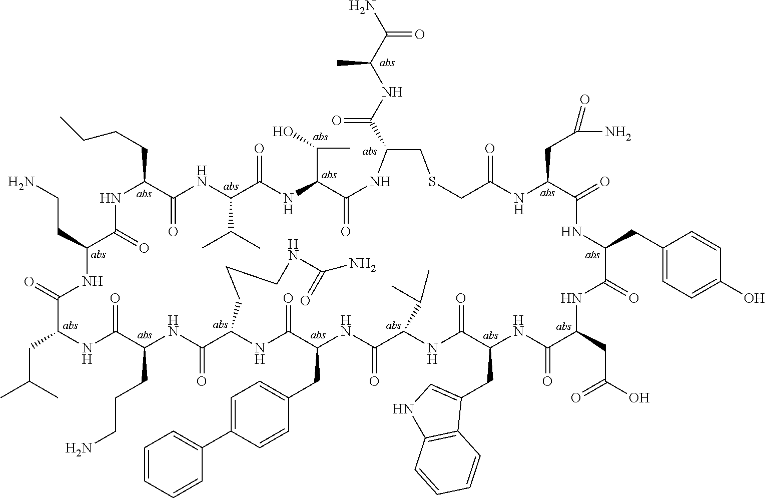 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1162 was prepared on a 50 mol scale. The yield of the product was 0.7 mg, and its estimated purity by LCMS analysis was 94.7%. Analysis condition B: Retention time=1.39 min; ESI-MS(+) m/z [M+2H]²⁺: 966.

### Preparation of Compound 1163

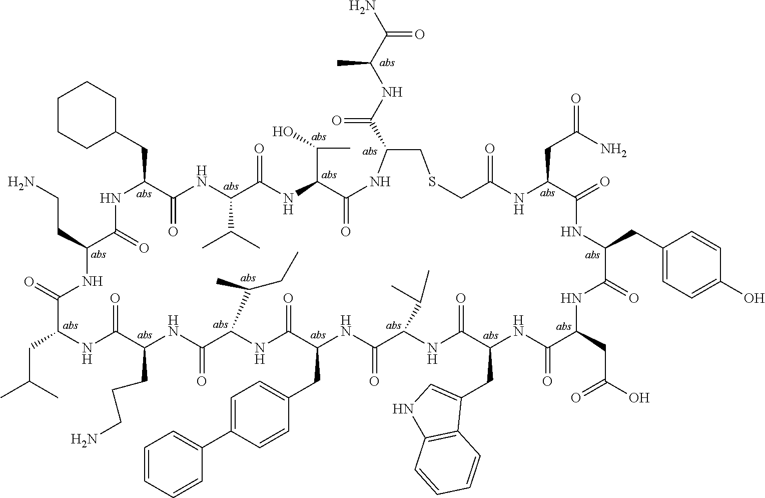 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1163 was prepared on a 50 μmol scale. The yield of the product was 1.4 mg, and its estimated purity by LCMS analysis was 97.7%. Analysis condition A: Retention time=1.73 min; ESI-MS(+) m/z [M+2H]²⁺: 964.1.

### Preparation of Compound 1164

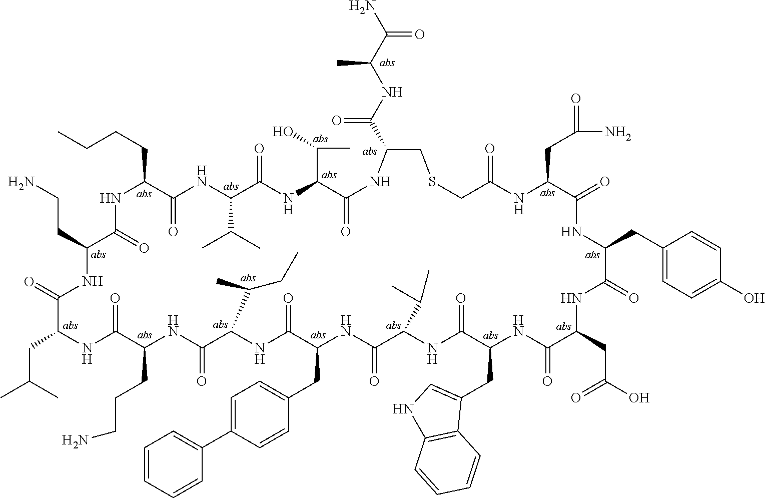 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1165 was prepared on a 50 mol scale. The yield of the product was 1.6 mg, and its estimated purity by LCMS analysis was 82.7%. Analysis condition A: Retention time=1.57 min: ESI-MS(+) m/z [M+2H]²⁺: 10384.

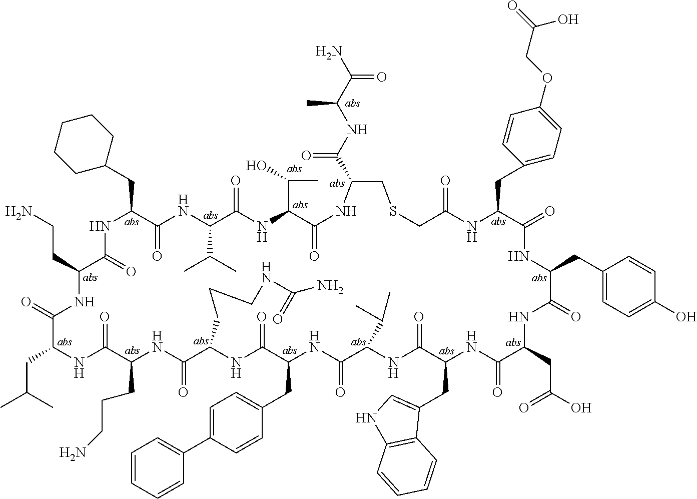 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1165 was prepared on a 50 μmol scale. The yield of the product was 16 mg, and its estimated purity by LCMS analysis was 82.7%. Analysis condition A: Retention time=1.57 min; ESI-MS(+) m/z [M+2H]²⁺: 1038.9.

### Preparation of Compound 1166

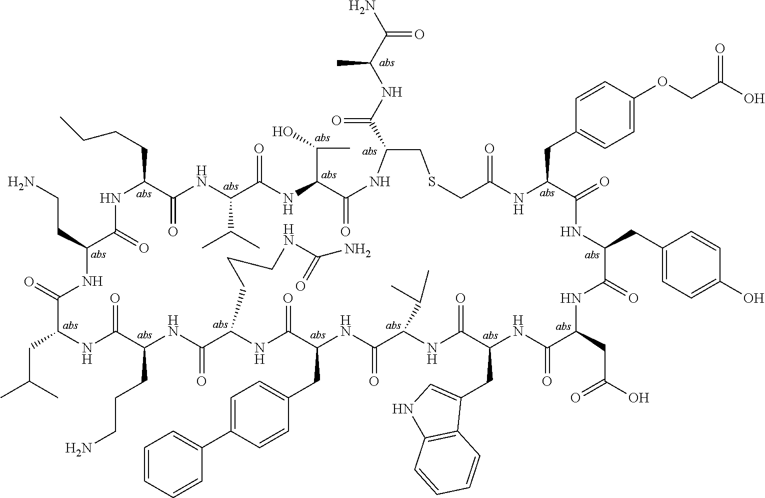 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1166 was prepared on a 50 μmol scale. The yield of the product was 2.6 mg, and its estimated purity by LCMS analysis was 90%. Analysis condition A: Retention time=1.52, 1.62 min; ESI-MS(+) m/z [M+2H]²⁺: 1020.

### Preparation of Compound 1167

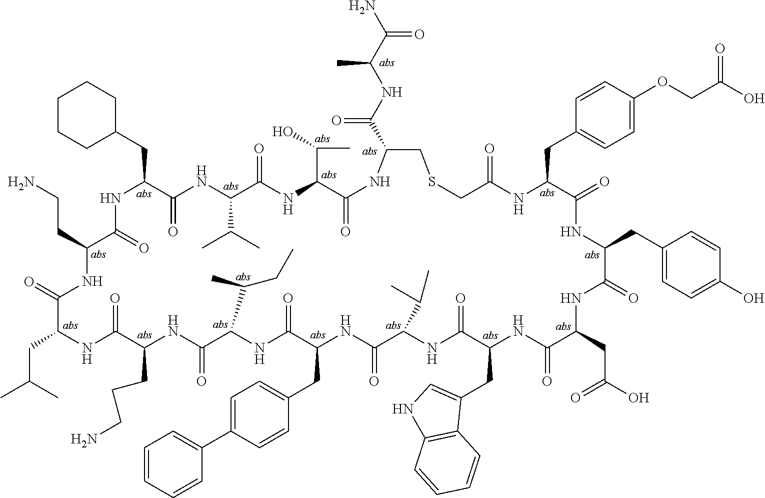 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1167 was prepared on a 50 μmol scale. The yield of the product was 14.7 mg, and its estimated purity by LCMS analysis was 87.3%. Analysis condition A: Retention time=1.96 min; ESI-MS(+) m/z [M+2H]²⁺: 1017.1.

### Preparation of Compound 1168

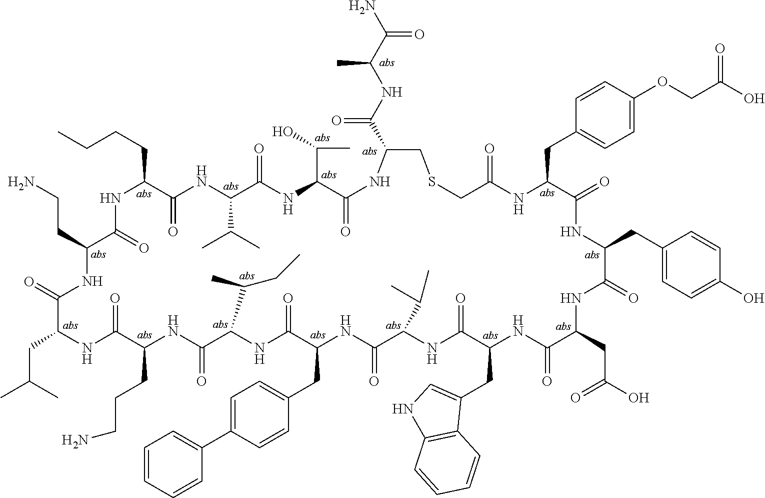 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1168 was prepared on a 50 μmol scale. The yield of the product was 1.6 mg, and its estimated purity by LCMS analysis was 92.1%. Analysis condition A: Retention time=1.78 min; ESI-MS(+) m/z [M+2H]²⁺: 997.2.

### Preparation of Compound 1169

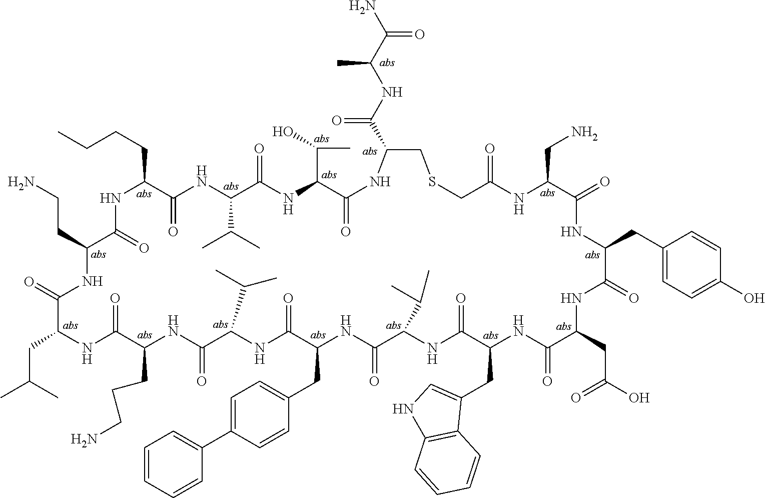 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1169 was prepared on a 50 μmol scale. The yield of the product was 3.8 mg, and its estimated purity by LCMS analysis was 98%. Analysis condition A: Retention time=1.35 min; ESI-MS(+) m/z [M+2H]²⁺: 922.8.

### Preparation of Compound 1170

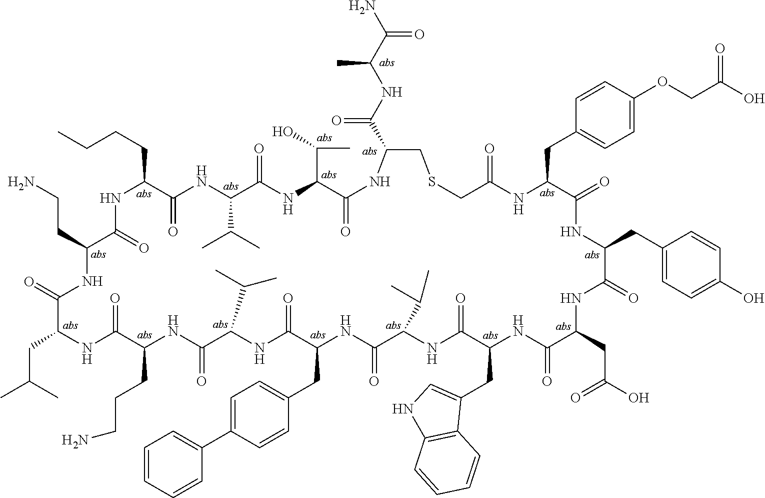 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1170 was prepared on a 50 mol scale. The yield of the product was 4.5 mg, and its estimated purity by LCMS analysis was 98.5%. Analysis condition B: Retention time=1.49 min; ESI-MS(+) m/z [M+2H]²⁺: 990.2.

### Preparation of Compound 1171

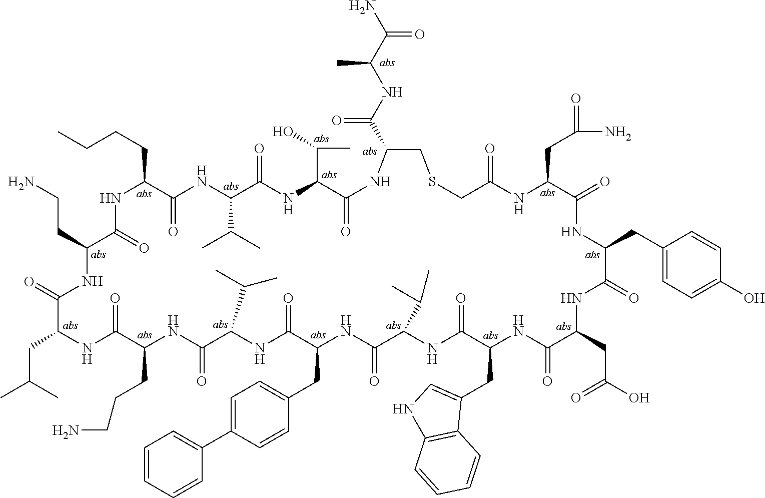 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1171 was prepared on a 50 μmol scale. The yield of the product was 1.4 mg, and its estimated purity by LCMS analysis was 85.1%. Analysis condition A: Retention time=1.54 min; ESI-MS(+) m/z [M+2H]²⁺: 936.5.

### Preparation of Compound 1172

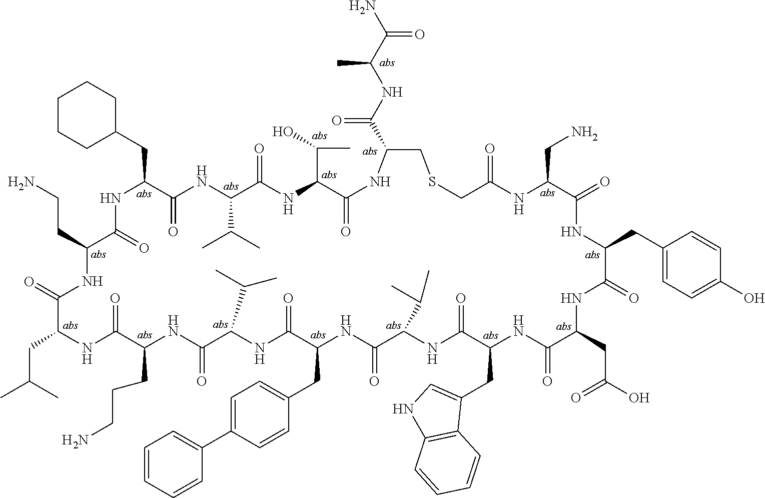 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1172 was prepared on a 50 mol scale. The yield of the product was 5 mg, and its estimated purity by LCMS analysis was 93.5%. Analysis condition A: Retention time=1.6 min; ESI-MS(+) m/z [M+2H]²⁺: 943.2.

### Preparation of Compound 1173

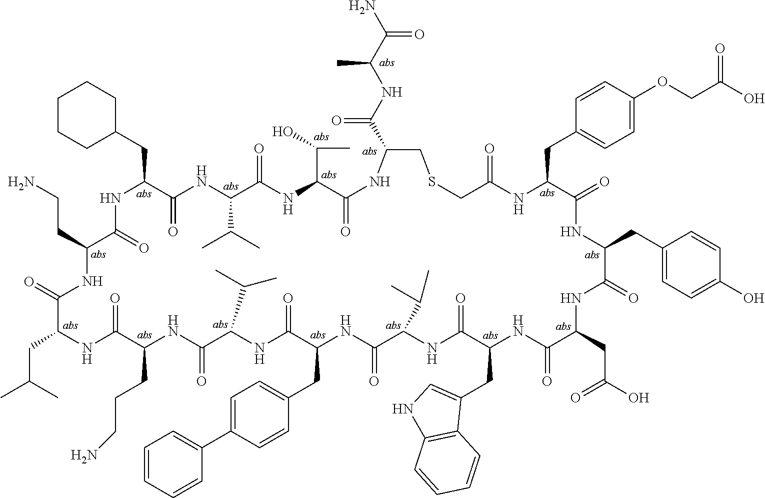 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1173 was prepared on a 50 μmol scale. The yield of the product was 7.5 mg, and its estimated purity by LCMS analysis was 86.6%. Analysis condition A: Retention time=1.87 min; ESI-MS(+) m/z [M+2H]²⁺: 1010.

### Preparation of Compound 1174

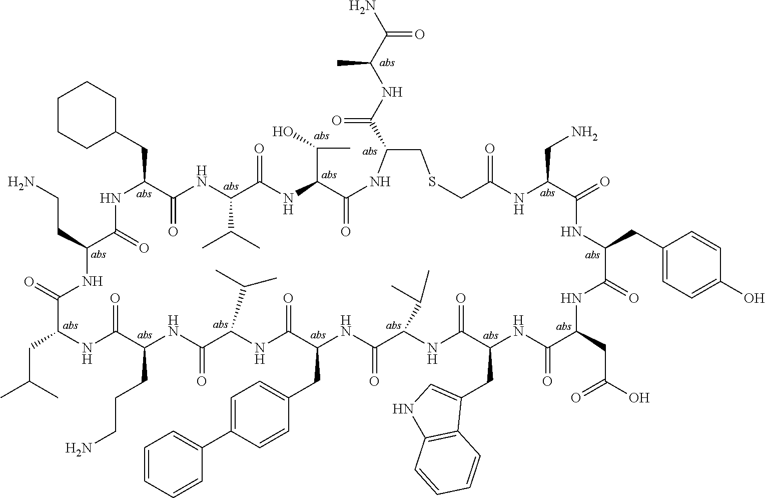 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1174 was prepared on a 50 mol scale. The yield of the product was 1.2 mg, and its estimated purity by LCMS analysis was 96.9%. Analysis condition B: Retention time=1.52 min; ESI-MS(+) m/z [M+2H]²⁺: 957.

### Preparation of Compound 1175

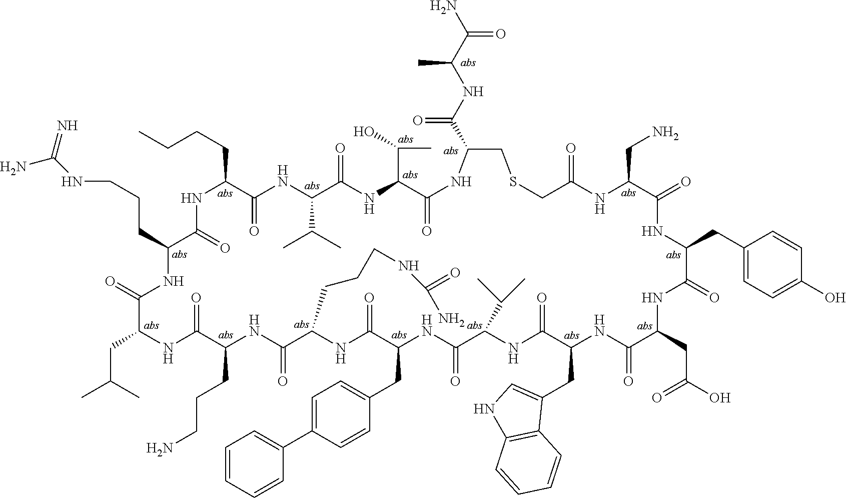 [SMILES: CCCC[C@@H]1NC(=O)[C@H](CCCNC(=N)N)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1175 was prepared on a 50 mol scale. The yield of the product was 12.3 mg, and its estimated purity by LCMS analysis was 82.9%. Analysis condition B: Retention time=1.29 min; ESI-MS(+) m/z [M+2H]²⁺: 980.1.

### Preparation of Compound 1176

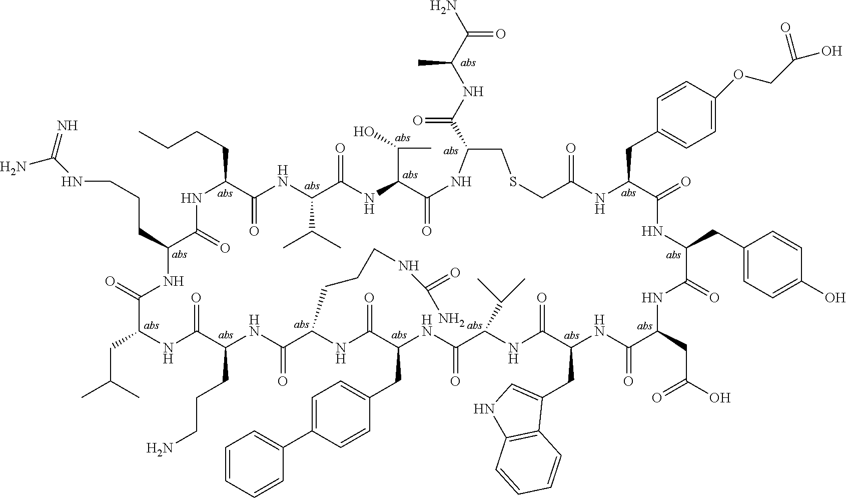 [SMILES: CCCC[C@@H]1NC(=O)[C@H](CCCNC(=N)N)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1176 was prepared on a 50 μmol scale. The yield of the product was 8 mg, and its estimated purity by LCMS analysis was 100%. Analysis condition A: Retention time=1.53 min; ESI-MS(+) m/z [M+2H]²⁺: 1047.1.

### Preparation of Compound 1177

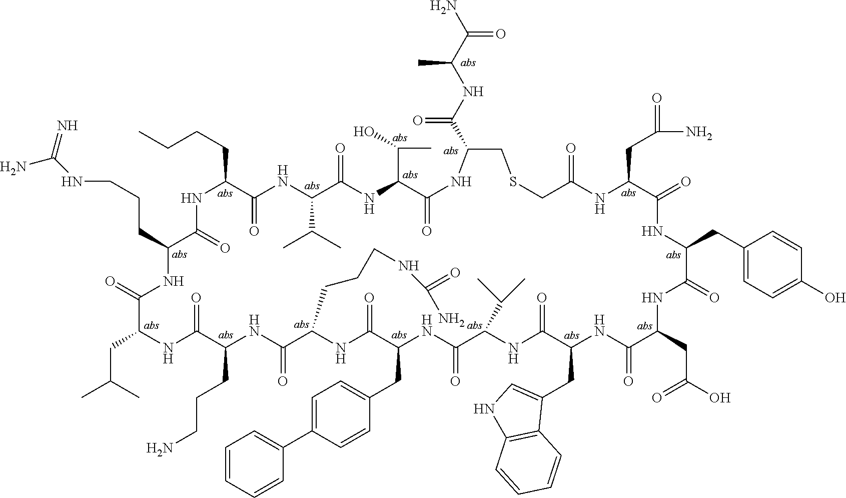 [SMILES: CCCC[C@@H]1NC(=O)[C@H](CCCNC(=N)N)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC1=O]

Compound 1177 was prepared on a 50 μmol scale. The yield of the product was 5.9 mg, and its estimated purity by LCMS analysis was 92.3%. Analysis condition A: Retention time=1.32, 1.35 min; ESI-MS(+) m/z [M+H]⁺: 1987.

### Preparation of Compound 1178

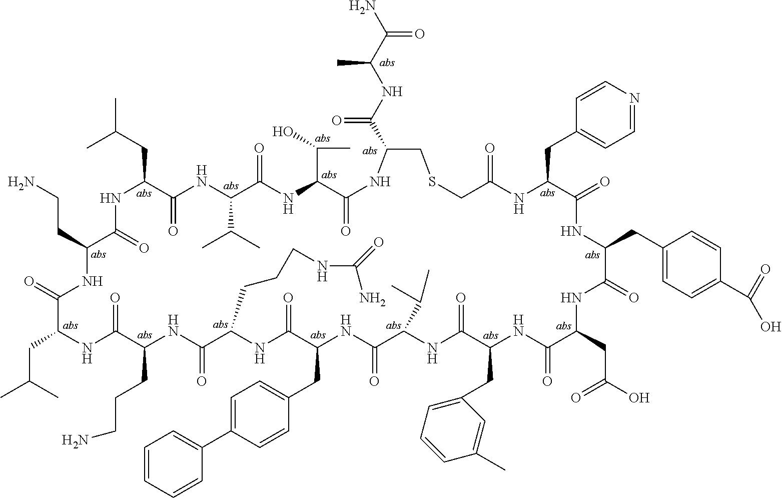 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1178 was prepared on a 50 μmol scale. The yield of the product was 3.1 mg, and its estimated purity by LCMS analysis was 91.5%. Analysis condition B: Retention time=1.32 min; ESI-MS(+) m/z [M+3H]³⁺: 656.2.

### Preparation of Compound 1179

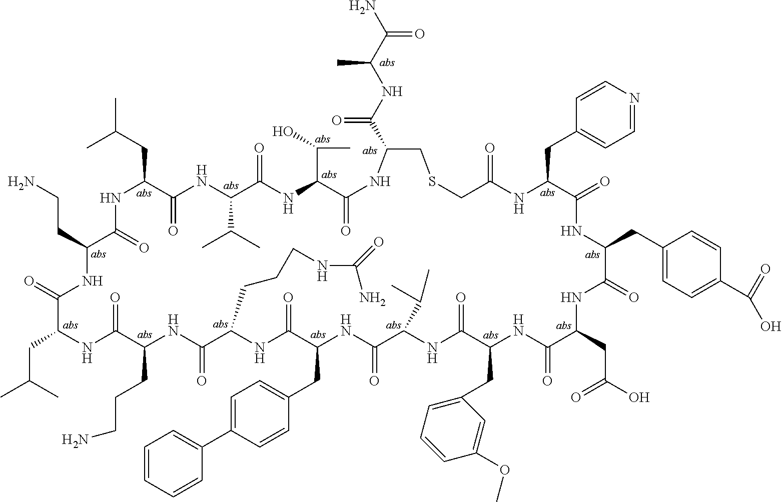 [SMILES: COc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1179 was prepared on a 50 μmol scale. The yield of the product was 2.7 mg, and its estimated purity by LCMS analysis was 94.1%. Analysis condition B: Retention time=1.39 min; ESI-MS(+) m/z [M+2H]²⁺: 992.2.

### Preparation of Compound 1180

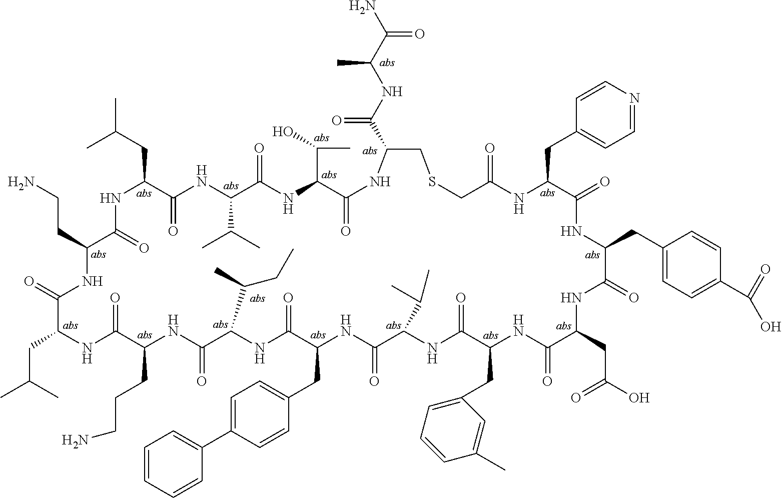 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1180 was prepared on a 50 μmol scale. The yield of the product was 1.7 mg, and its estimated purity by LCMS analysis was 88.9%. Analysis condition B: Retention time=1.39 min; ESI-MS(+) m/z [M+2H]²⁺: 962.2.

### Preparation of Compound 1181

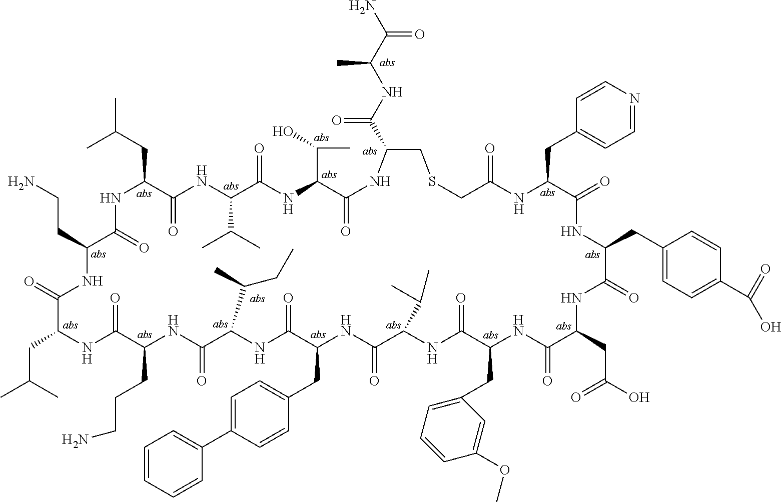 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(OC)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1181 was prepared on a 50 μmol scale. The yield of the product was 1.4 mg, and its estimated purity by LCMS analysis was 96.9%. Analysis condition A: Retention time=1.49 min; ESI-MS(+) m/z [M+3H]³⁺: 647.

### Preparation of Compound 1182

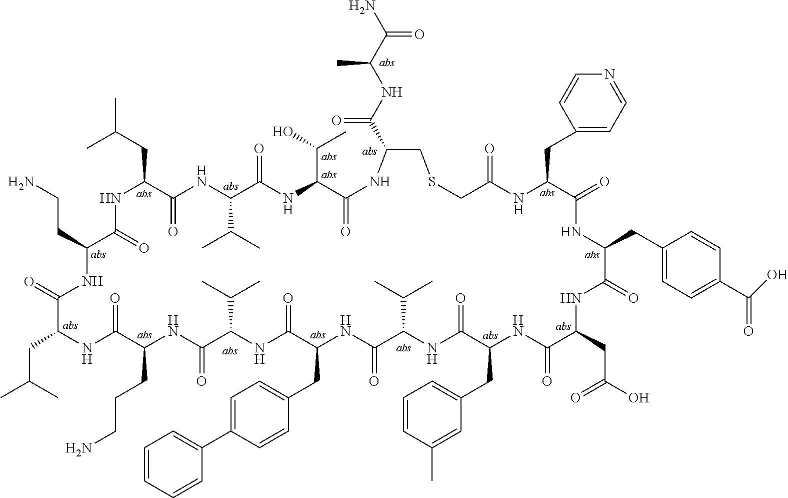 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1182 was prepared on a 50 μmol scale. The yield of the product was 1.6 mg, and its estimated purity by LCMS analysis was 100%. Analysis condition A: Retention time=1.5 min; ESI-MS(+) m/z [M+3H]³⁺: 637.13. Analysis condition B: Retention time=1.37 min; ESI-MS(+) m/z [M+3H]³⁺: 637.19.

### Preparation of Compound 1183

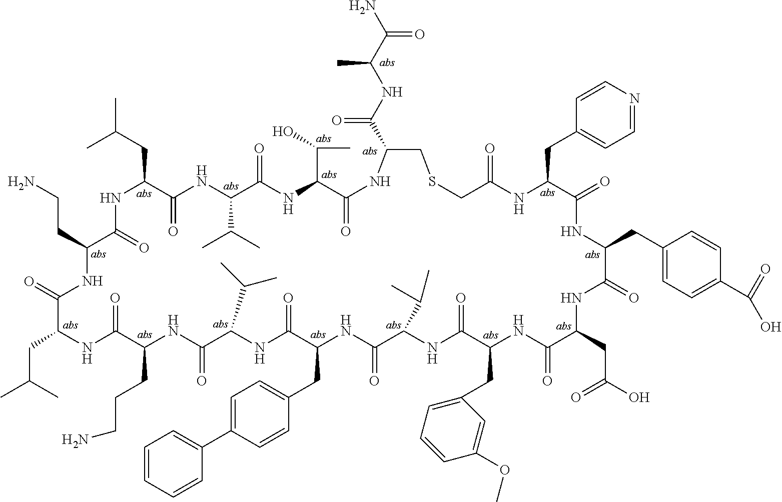 [SMILES: COc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1183 was prepared on a 50 μmol scale. The yield of the product was 1.6 mg, and its estimated purity by LCMS analysis was 91.3%. Analysis condition A: Retention time=1.52 min; ESI-MS(+) m/z [M+2H]²⁺: 963.1.

### Preparation of Compound 1184

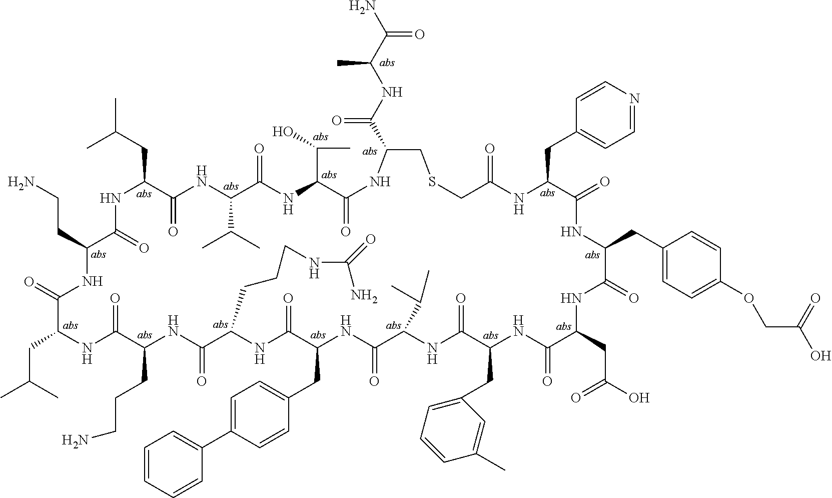 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1184 was prepared on a 50 μmol scale. The yield of the product was 5.3 mg, and its estimated purity by LCMS analysis was 100%. Analysis condition A: Retention time=1.47 min; ESI-MS(+) m/z [M+H]⁺: 1996.7.

### Preparation of Compound 1185

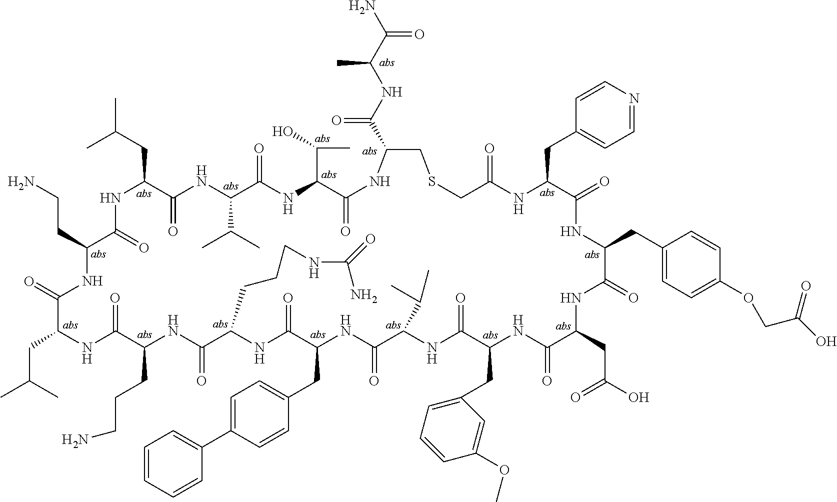 [SMILES: COc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1185 was prepared on a 50 μmol scale. The yield of the product was 8.2 mg, and its estimated purity by LCMS analysis was 94.1%. Analysis condition B: Retention time=1.39 min; ESI-MS(+) m/z [M+2H]²⁺: 1007.3.

### Preparation of Compound 1186

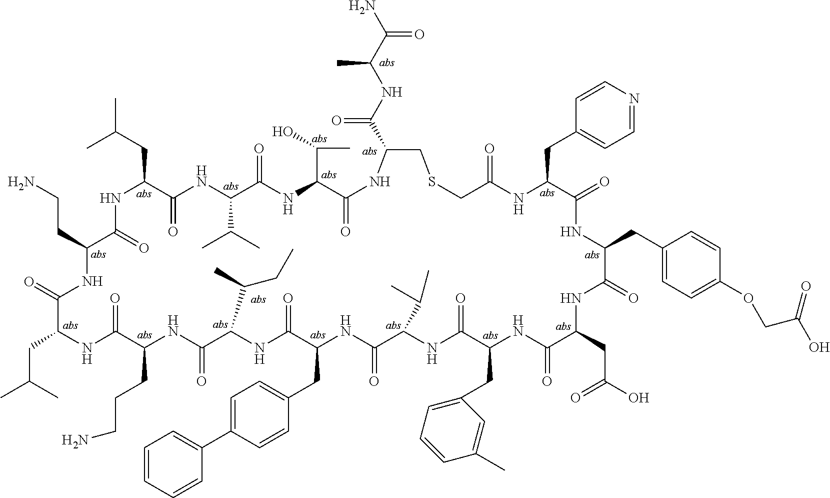 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1186 was prepared on a 50 μmol scale. The yield of the product was 1.9 mg, and its estimated purity by LCMS analysis was 89.3%. Analysis condition B: Retention time=1.38 min; ESI-MS(+) m/z [M+3H]³⁺: 652.1.

### Preparation of Compound 1187

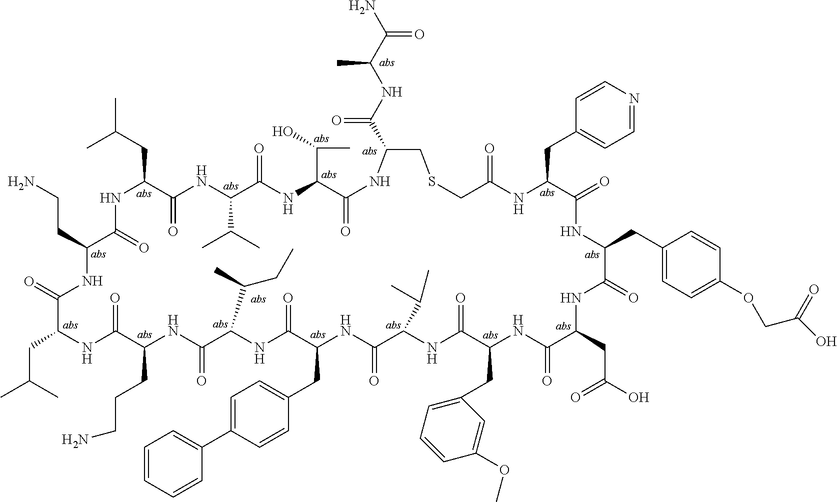 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(OC)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1187 was prepared on a 50 mol scale. The yield of the product was 1.8 mg, and its estimated purity by LCMS analysis was 90.8%. Analysis condition B: Retention time=1.35 min; ESI-MS(+) m/z [M+2H]²⁺: 985.1.

### Preparation of Compound 1188

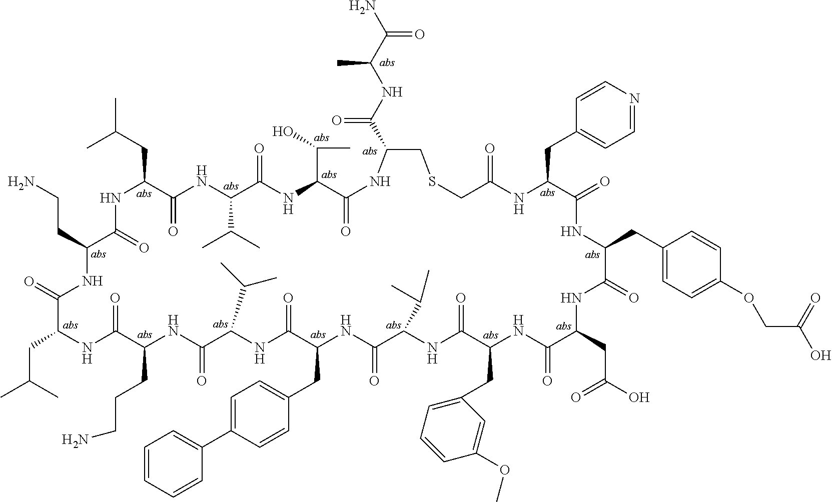 [SMILES: COc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1188 was prepared on a 50 μmol scale. The yield of the product was 2.2 mg, and its estimated purity by LCMS analysis was 92.7%. Analysis condition A: Retention time=1.47 min; ESI-MS(+) m/z [M+3H]³⁺: 653.

### Preparation of Compound 1189

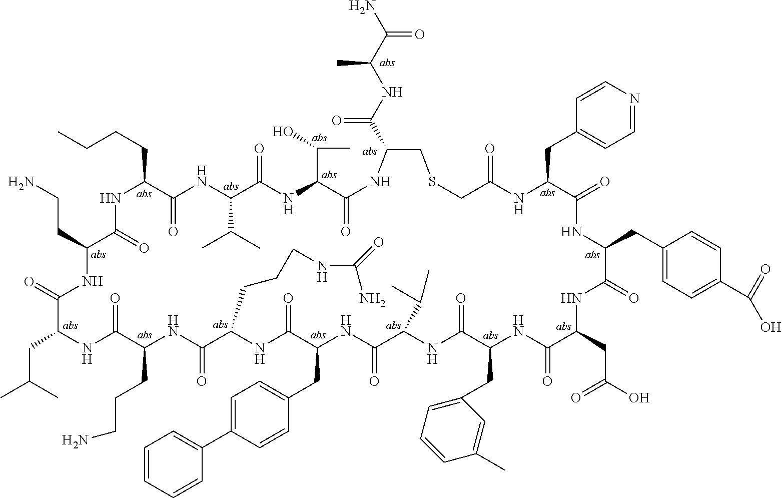 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1189 was prepared on a 50 μmol scale. The yield of the product was 4.1 mg, and its estimated purity by LCMS analysis was 89%. Analysis condition B: Retention time=1.33 min; ESI-MS(+) m/z [M+3H]³⁺: 656.7.

### Preparation of Compound 1190

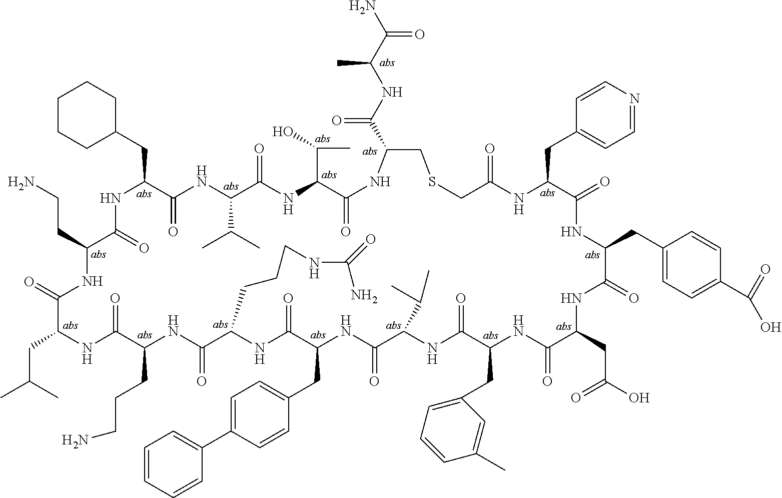 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1190 was prepared on a 50 μmol scale. The yield of the product was 8.5 mg, and its estimated purity by LCMS analysis was 95.1%. Analysis condition B: Retention time=1.43 min; ESI-MS(+) m/z [M+2H]²⁺: 670.2.

### Preparation of Compound 1191

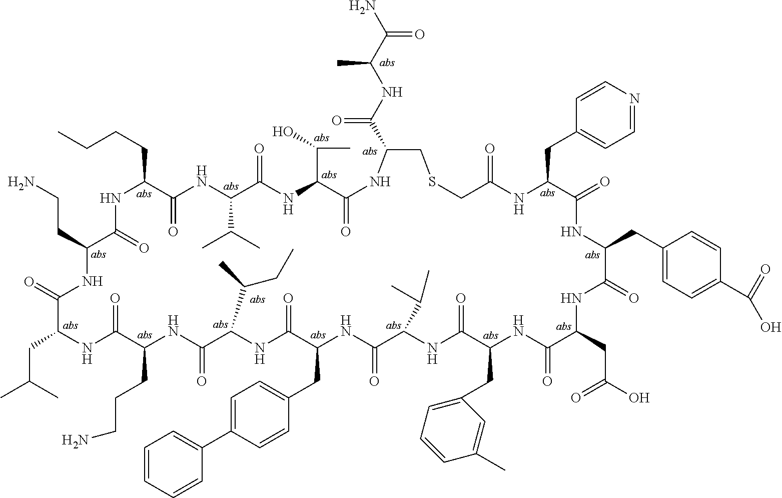 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1191 was prepared on a 50 μmol scale. The yield of the product was 2.3 mg, and its estimated purity by LCMS analysis was 97.5%. Analysis condition A: Retention time=1.57 min; ESI-MS(+) m/z [M+2H]²⁺: 642.

### Preparation of Compound 1192

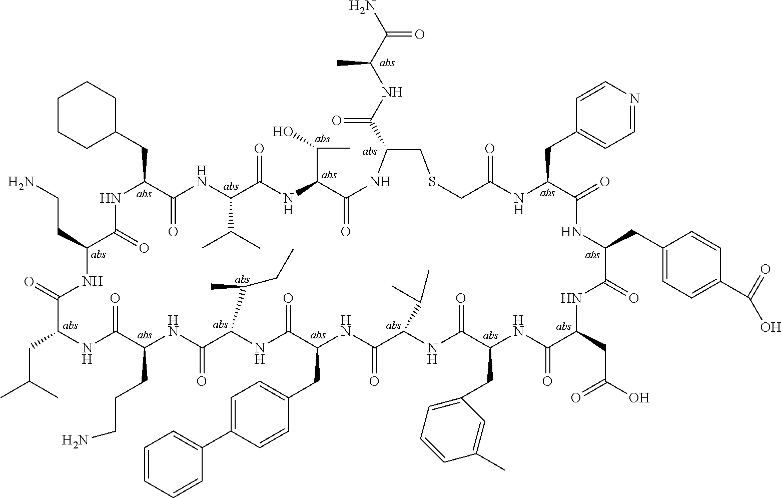 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1192 was prepared on a 50 μmol scale. The yield of the product was 4.6 mg, and its estimated purity by LCMS analysis was 95.1%. Analysis condition A: Retention time=1.78 min; ESI-MS(+) m/z [M+2H]²⁺: 1963.2.

### Preparation of Compound 1193

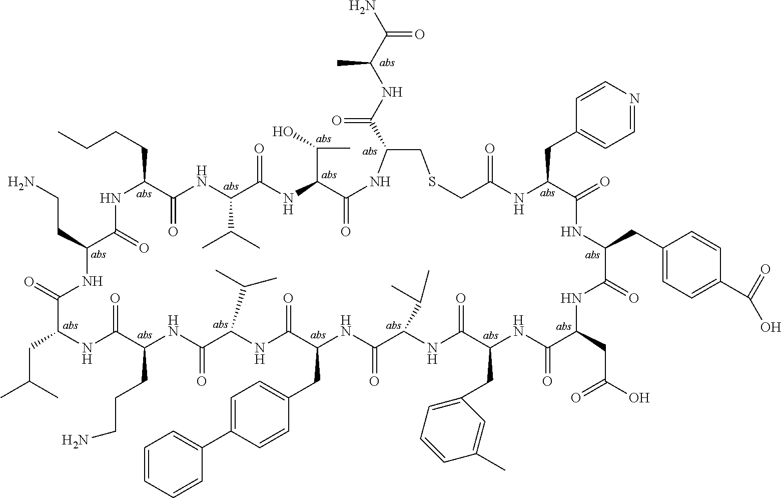 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1193 was prepared on a 50 μmol scale. The yield of the product was 1.6 mg, and its estimated purity by LCMS analysis was 79.5%. Analysis condition B: Retention time=1.39 min; ESI-MS(+) m/z [M+3H]³⁺: 637.

### Preparation of Compound 1194

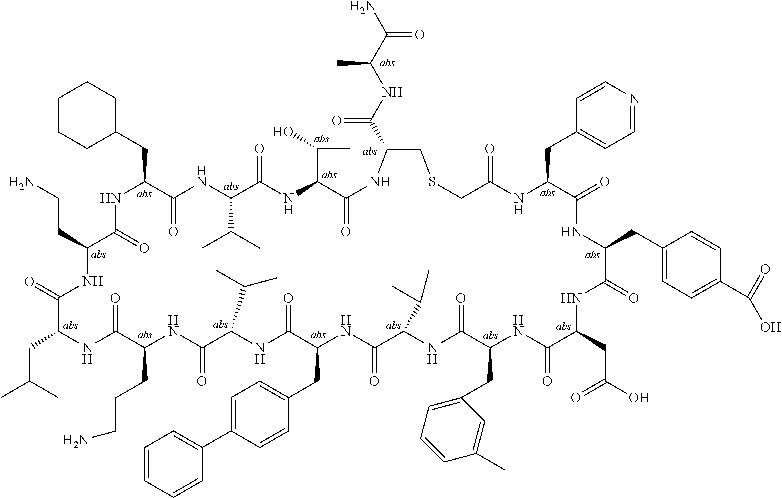 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1194 was prepared on a 50 mol scale. The yield of the product was 14 mg, and its estimated purity by LCMS analysis was 96.9%. Analysis condition A: Retention time=1.74 min; ESI-MS(+) m/z [M+H]⁺: 1948.7.

### Preparation of Compound 1195

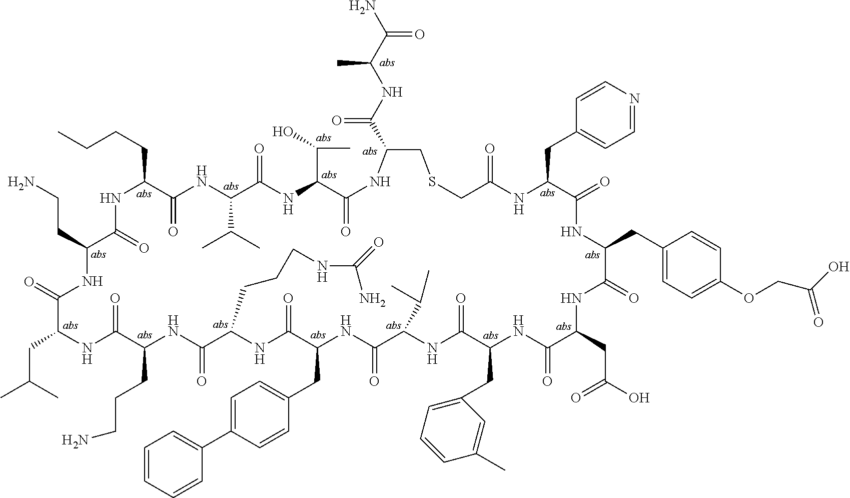 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1195 was prepared on a 50 μmol scale. The yield of the product was 2.6 mg, and its estimated purity by LCMS analysis was 97.6%. Analysis condition B: Retention time=1.34 min; ESI-MS(+) m/z [M+3H]³⁺: 667.

### Preparation of Compound 1196

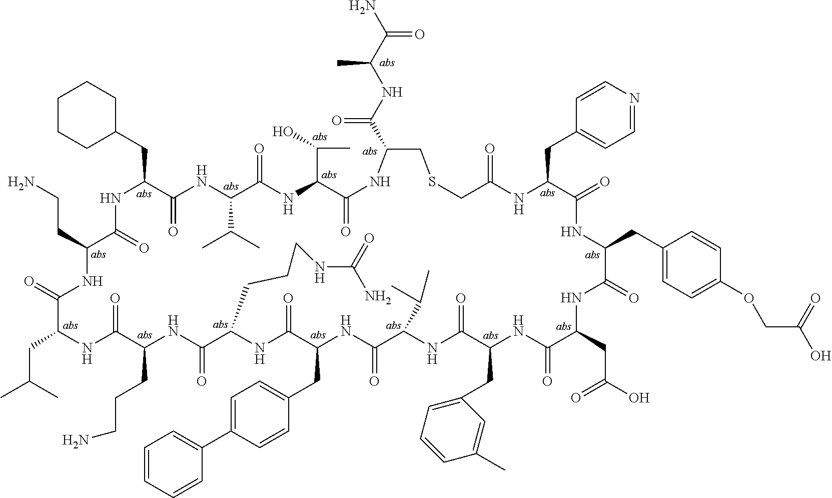 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1196 was prepared on a 50 μmol scale. The yield of the product was 11.9 mg, and its estimated purity by LCMS analysis was 92%. Analysis condition A: Retention time=1.49 min; ESI-MS(+) m/z [M+3H]³⁺: 680.

### Preparation of Compound 1197

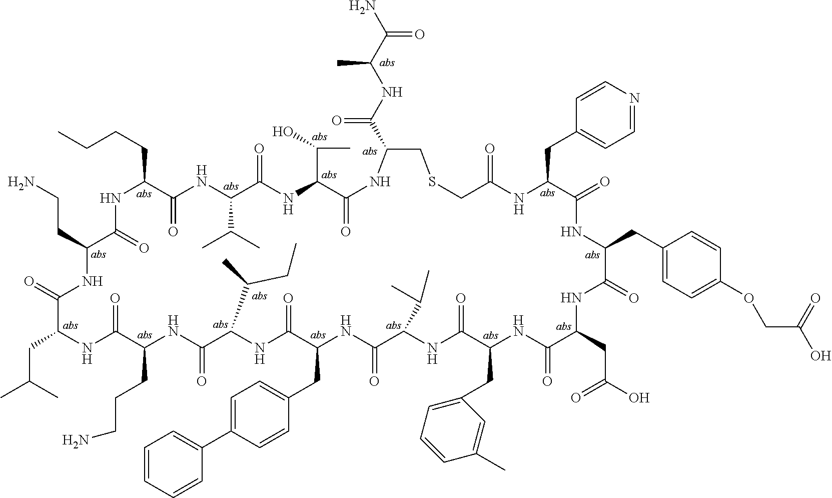 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1197 was prepared on a 50 μmol scale. The yield of the product was 2 mg, and its estimated purity by LCMS analysis was 97.6%. Analysis condition A: Retention time=1.59 min; ESI-MS(+) m/z [M+3H]³⁺: 652.

### Preparation of Compound 1198

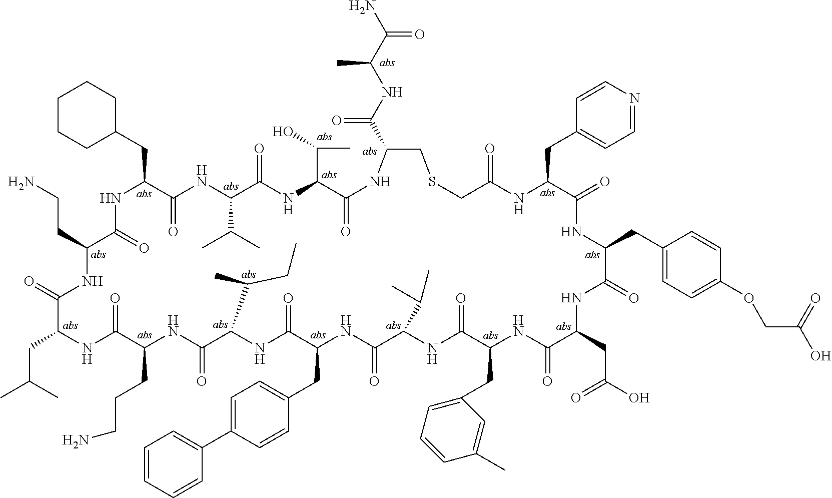 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1198 was prepared on a 5000 mol scale. The yield of the product was 9.5 mg, and its estimated purity by LCMS analysis was 88.1%. Analysis condition B: Retention time=1.49, 1.52 min; ESI-MS(+) m/z [M+3H]³⁺: 665.22, 665.12.

### Preparation of Compound 1199

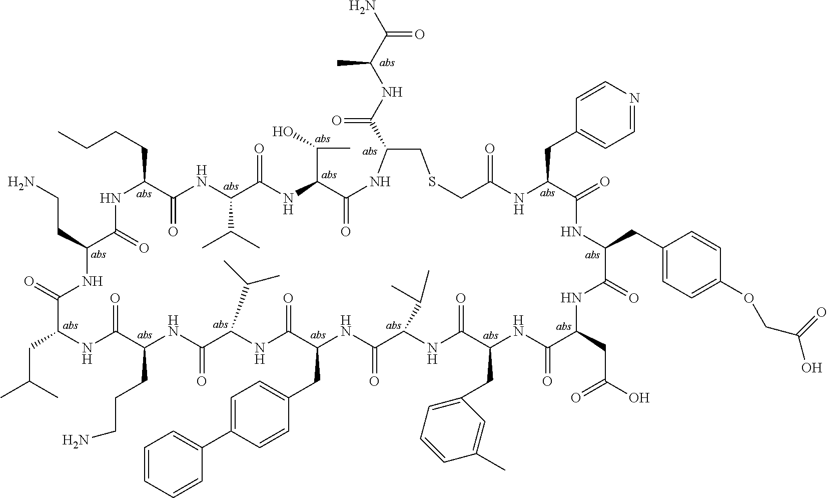 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1199 was prepared on a 50 μmol scale. The yield of the product was 1.8 mg, and its estimated purity by LCMS analysis was 94.3%. Analysis condition B: Retention time=1.39 min; ESI-MS(+) m/z [M+2H]²⁺: 970.2.

### Preparation of Compound 1200

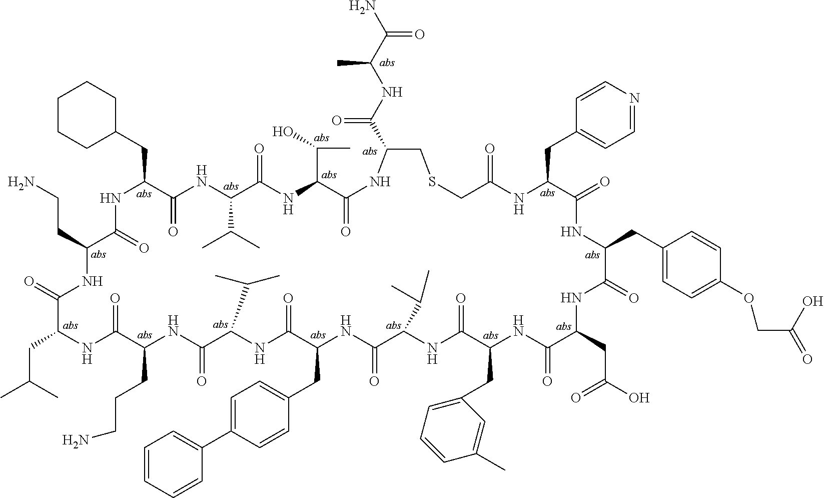 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1200 was prepared on a 50 μmol scale. The yield of the product was 3.1 mg, and its estimated purity by LCMS analysis was 93.6%. Analysis condition A: Retention time=1.67 min; ESI-MS(+) m/z [M+2H]²⁺: 990.1. Analysis condition B: Retention time=1.46 min; ESI-MS(+) m/z [M+2H]²⁺: 990.1.

### Preparation of Compound 1201

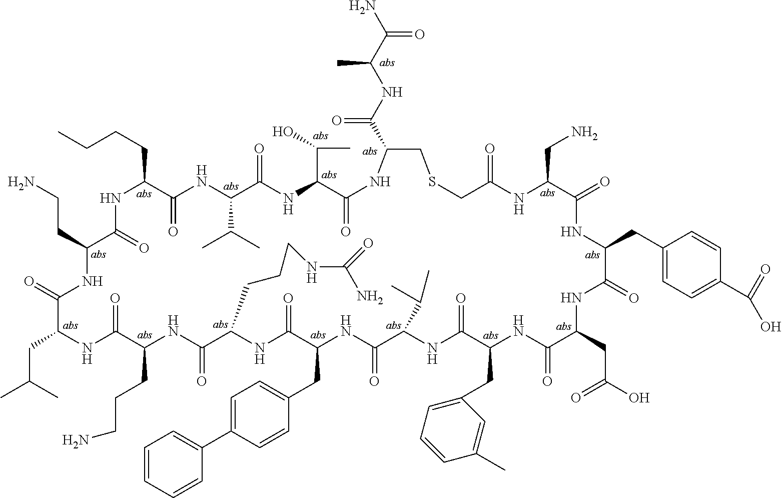 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1201 was prepared on a 50 μmol scale. The yield of the product was 2.4 mg, and its estimated purity by LCMS analysis was 100%. Analysis condition A: Retention time=1.44 min; ESI-MS(+) m/z [M+2H]²⁺: 953.1.

### Preparation of Compound 1202

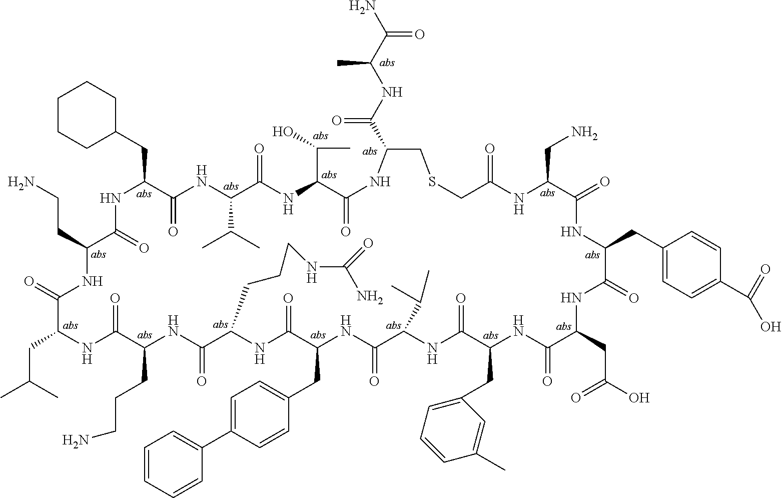 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1202 was prepared on a 50 mol scale. The yield of the product was 10.2 mg, and its estimated purity by LCMS analysis was 92.1%. Analysis condition B: Retention time=1.52 min; ESI-MS(+) m/z [M+H]⁺: 1946.3.

### Preparation of Compound 1203

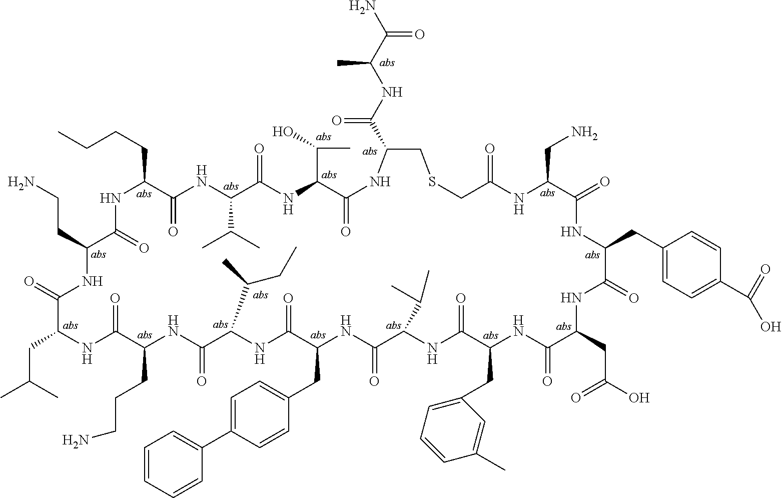 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1203 was prepared on a 50 μmol scale. The yield of the product was 1.6 mg, and its estimated purity by LCMS analysis was 92.1%. Analysis condition B: Retention time=1.49 min; ESI-MS(+) m/z [M+2H]²⁺: 932.1.

### Preparation of Compound 1204

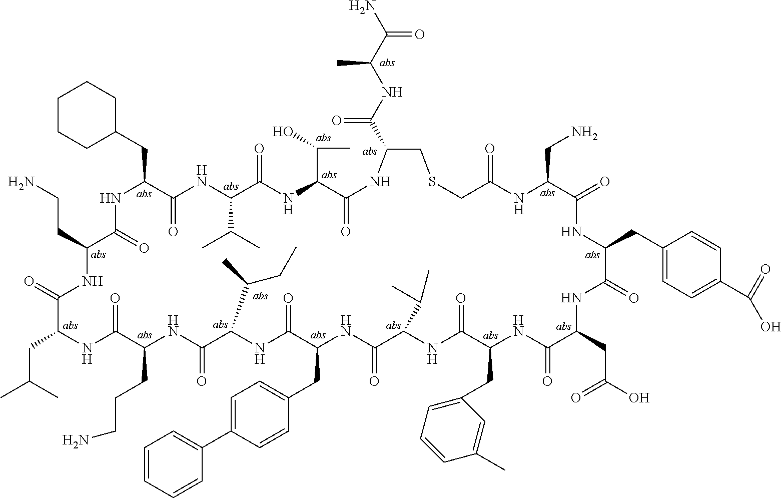 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1204 was prepared on a 50 μmol scale. The yield of the product was 6.2 mg, and its estimated purity by LCMS analysis was 91.6%. Analysis condition B: Retention time=1.56 min; ESI-MS(+) m/z [M+3H]³⁺: 634.4.

### Preparation of Compound 1205

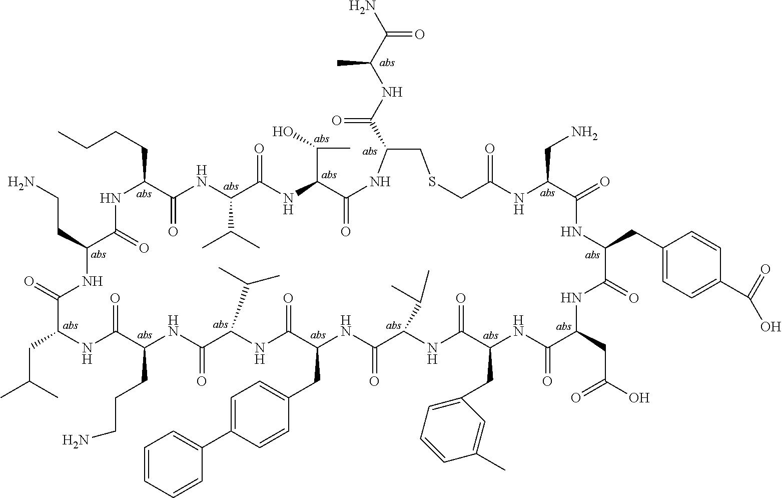 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1205 was prepared on a 50 μmol scale. The yield of the product was 1.7 mg, and its estimated purity by LCMS analysis was 95%. Analysis condition A: Retention time=1.52 min; ESI-MS(+) m/z [M+3H]³⁺: 616.9.

### Preparation of Compound 1206

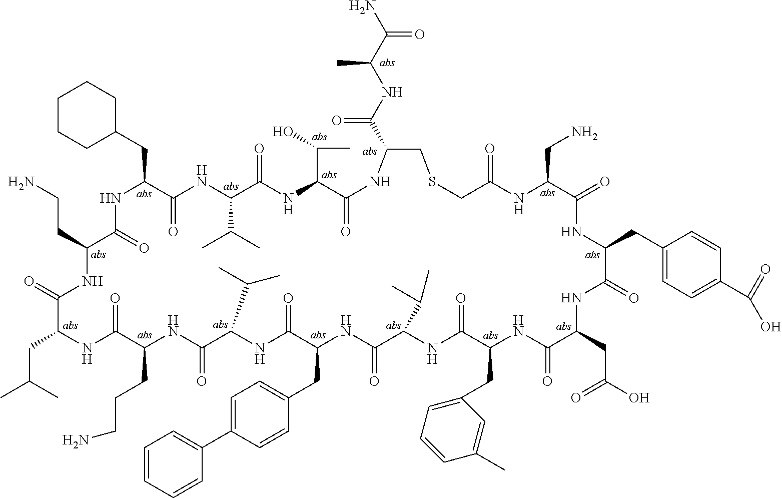 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1206 was prepared on a 50 μmol scale. The yield of the product was 4 mg, and its estimated purity by LCMS analysis was 100%. Analysis condition A: Retention time=1.65 min; ESI-MS(+) m/z [M+H]⁺: 1887.2.

### Preparation of Compound 1207

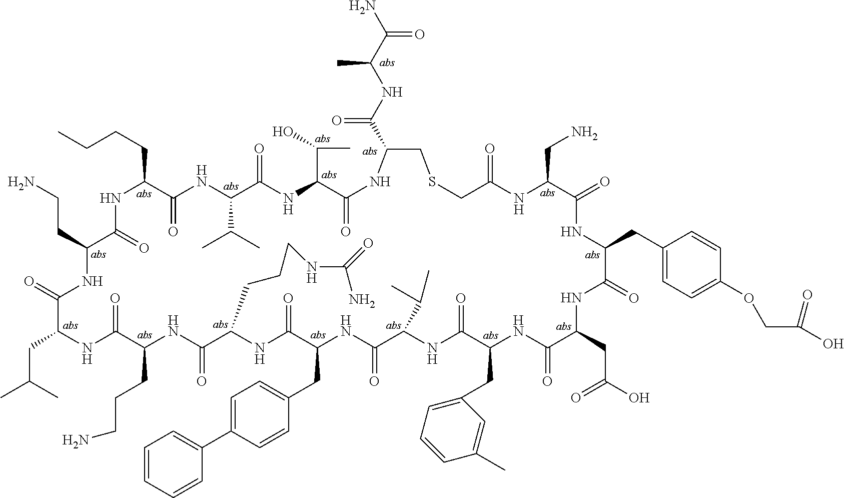 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1207 was prepared on a 50 μmol scale. The yield of the product was 3.5 mg, and its estimated purity by LCMS analysis was 96.2%. Analysis condition A: Retention time=1.35 min; ESI-MS(+) m/z [M+2H]²⁺: 968.3.

### Preparation of Compound 1208

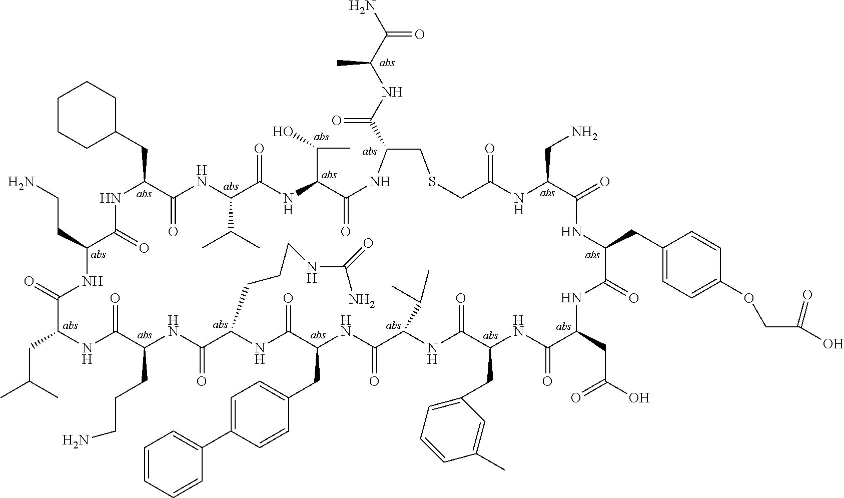 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1208 was prepared on a 50 μmol scale. The yield of the product was 5 mg, and its estimated purity by LCMS analysis was 92.5%. Analysis condition A: Retention time=1.47 min; ESI-MS(+) m/z [M+2H]²⁺: 988.2.

### Preparation of Compound 1209

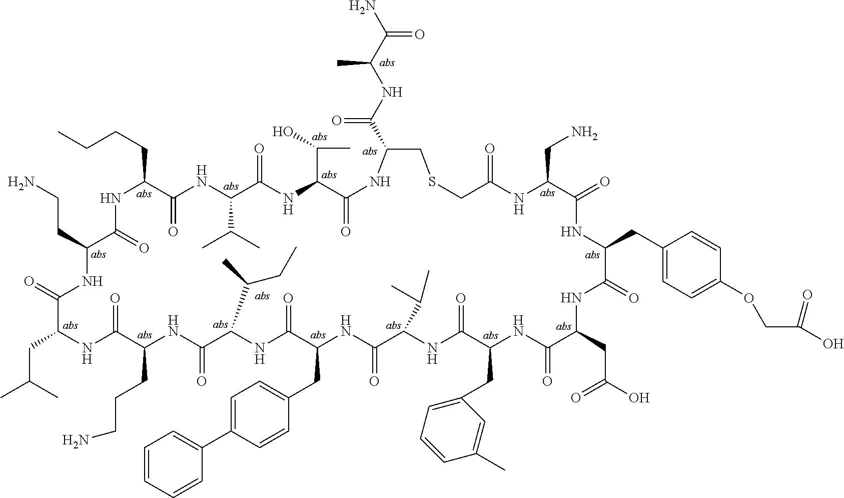 [SMILES: CCCC[C@@H]1NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1209 was prepared on a 50 μmol scale. The yield of the product was 2.4 mg, and its estimated purity by LCMS analysis was 86.3%. Analysis condition B: Retention time=1.4, 1.44 min; ESI-MS(+) m/z [M+3H]³⁺: 631.2, 631.34.

### Preparation of Compound 1210

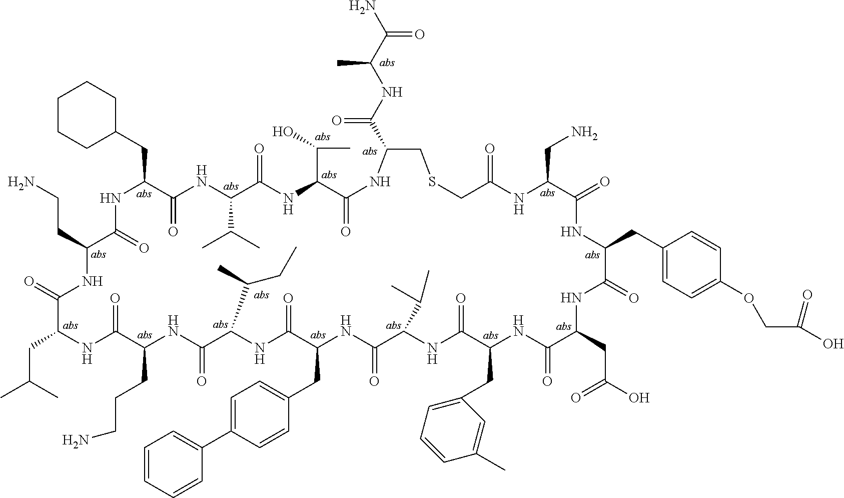 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1210 was prepared on a 50 mol scale. The yield of the product was 3.5 mg, and its estimated purity by LCMS analysis was 85.4%. Analysis condition B: Retention time=1.48 min; ESI-MS(+) m/z [M+2H]²⁺: 966.2.

### Preparation of Compound 1211

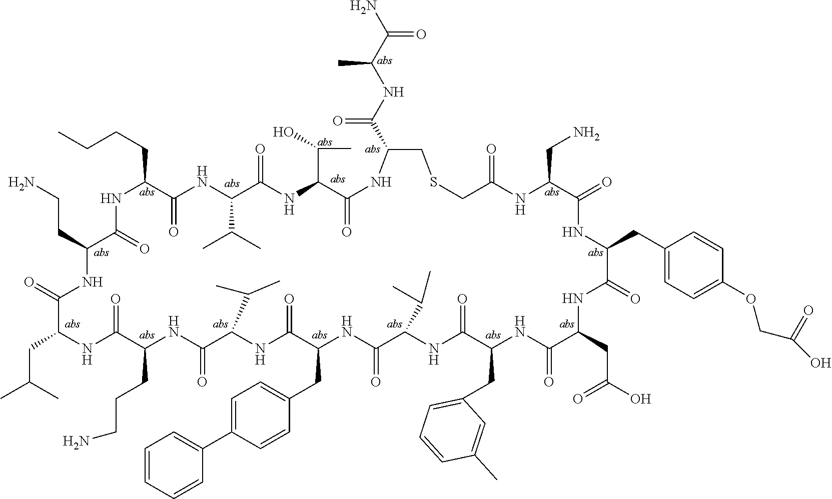 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1211 was prepared on a 50 μmol scale. The yield of the product was 1.4 mg, and its estimated purity by LCMS analysis was 83.9%. Analysis condition A: Retention time=1.43 min; ESI-MS(+) m/z [M+2H]²⁺: 939.

### Preparation of Compound 1212

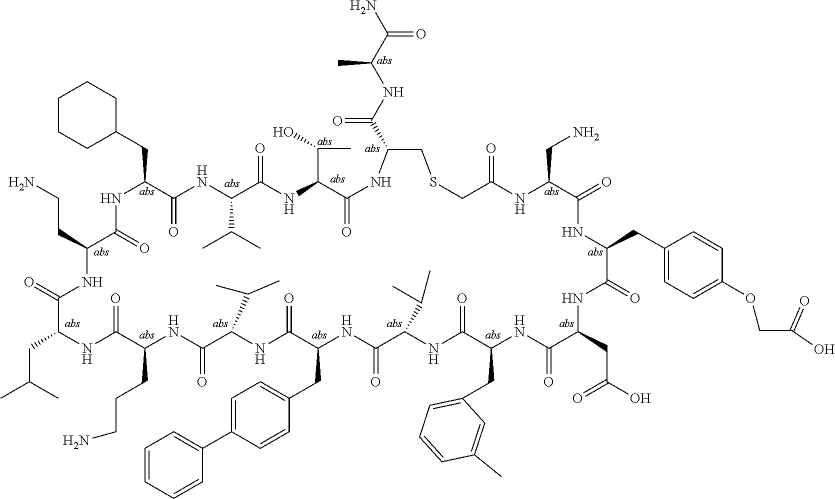 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1212 was prepared on a 50 μmol scale. The yield of the product was 5.2 mg, and its estimated purity by LCMS analysis was 87.9%. Analysis condition A: Retention time=1.56 min; ESI-MS(+) m/z [M+3H]³⁺: 639.6.

### Preparation of Compound 1213

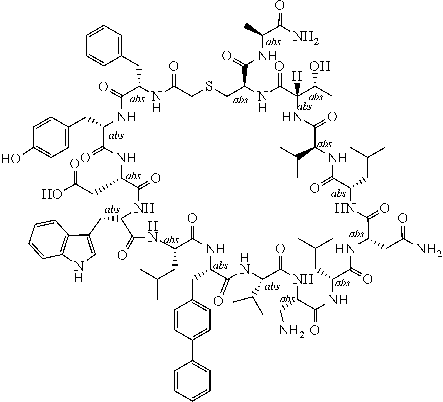

Compound 1213 was prepared on a 50 mol scale. The yield of the product was 14.7 mg, and its estimated purity by LCMS analysis was 90.2%. Analysis condition B: Retention time=2.95 min; ESI-MS(+) m/z [M+3H]³⁺: 636.

### Preparation of Compound 1214

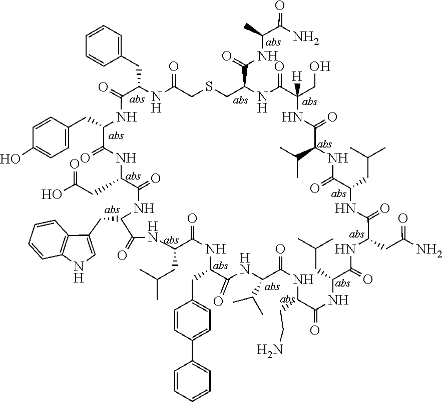

Compound 1214 was prepared on a 50 μmol scale. The yield of the product was 15.2 mg, and its estimated purity by LCMS analysis was 94.7%. Analysis condition B: Retention time=1.84 min; ESI-MS(+) m/z [M+2H]²⁺: 953.1.

### Preparation of Compound 1215

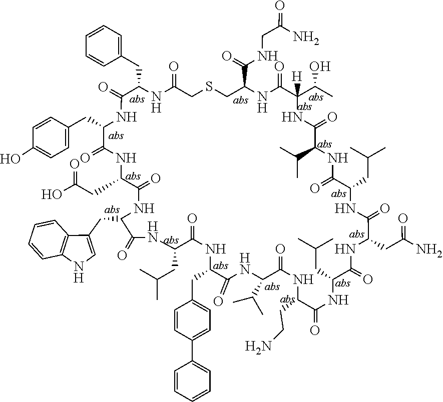

Compound 1215 was prepared on a 50 μmol scale. The yield of the product was 14.3 mg, and its estimated purity by LCMS analysis was 95.2%. Analysis condition B: Retention time=1.81 min; ESI-MS(+) m/z [M+H]⁺: 1905.1.

### Preparation of Compound 1216

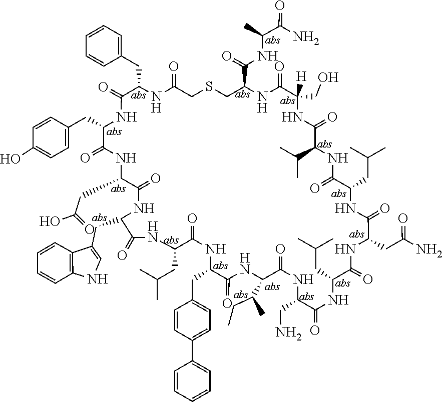

Compound 1216 was prepared on a 50 mol scale. The yield of the product was 7.6 mg, and its estimated purity by LCMS analysis was 94.6%. Analysis condition B: Retention time=1.84 min; ESI-MS(+) m/z [M+2H]²⁺: 953.2.

### Preparation of Compound 1217

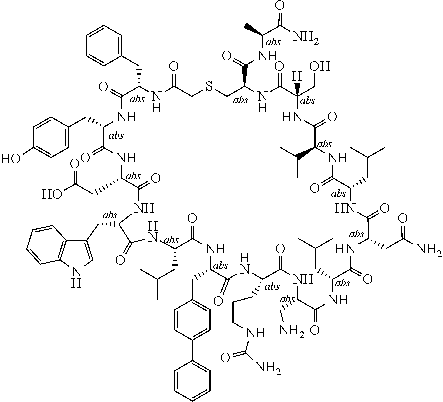

Compound 1217 was prepared on a 50 μmol scale. The yield of the product was 15 mg, and its estimated purity by LCMS analysis was 99.2%. Analysis condition A: Retention time=1.73 min; ESI-MS(+) m/z [M+H]⁺: 1949.1.

### Preparation of Compound 1218

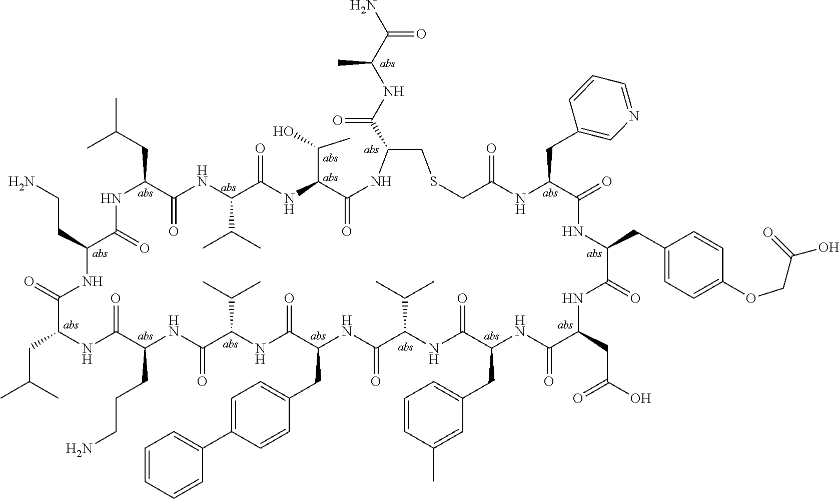 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1218 was prepared on a 50 μmol scale. The yield of the product was 4.5 mg, and its estimated purity by LCMS analysis was 99.3%. Analysis condition B: Retention time=1.34, 1.37 min; ESI-MS(+) m/z [M+2H]²⁺: 971.

### Preparation of Compound 1219

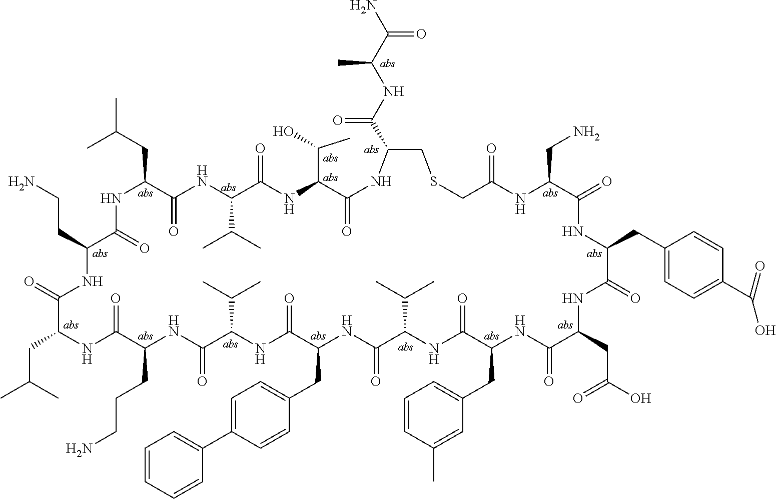 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1219 was prepared on a 50 mol scale. The yield of the product was 6.9 mg, and its estimated purity by LCMS analysis was 92.2%. Analysis condition B: Retention time=1.44 min; ESI-MS(+) m/z [M+H]⁺: 1846.6.

### Preparation of Compound 1220

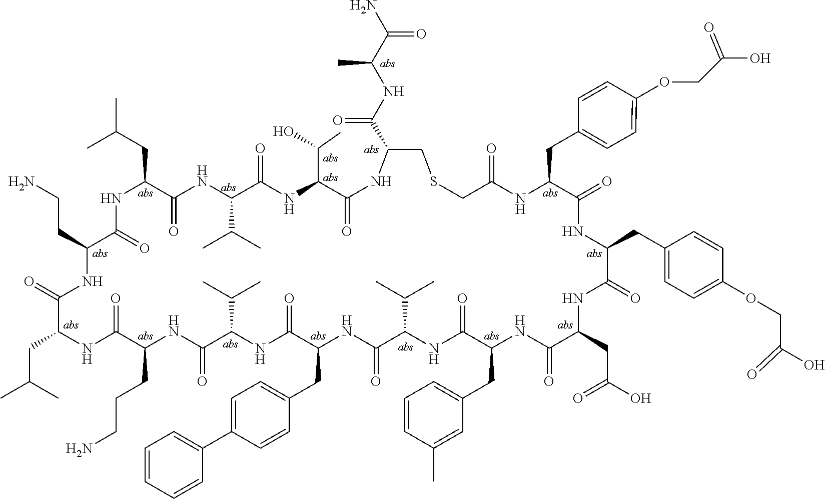 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1220 was prepared on a 50 mol scale. The yield of the product was 13.7 mg, and its estimated purity by LCMS analysis was 98.9%. Analysis condition B: Retention time=1.59 min; ESI-MS(+) m/z [M+2H]²⁺: 1007.

### Preparation of Compound 1221

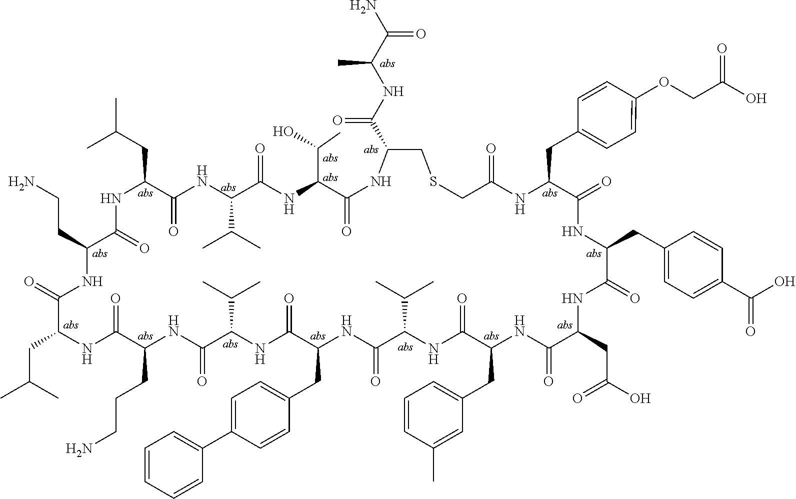 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1221 was prepared on a 50 μmol scale. The yield of the product was 9 mg, and its estimated purity by LCMS analysis was 94.1%. Analysis condition B: Retention time=1.58 min; ESI-MS(+) m/z [M+H]⁺: 1982.1.

### Preparation of Compound 1222

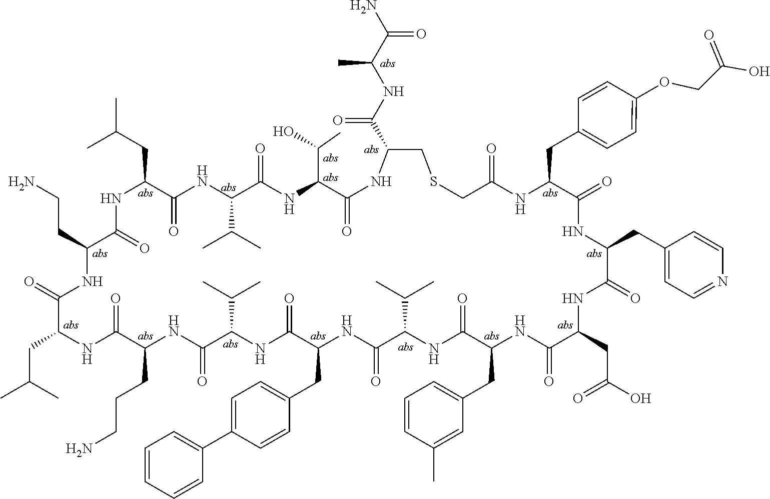 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1222 was prepared on a 50 mol scale. The yield of the product was 0.8 mg, and its estimated purity by LCMS analysis was 95.2%. Analysis condition A: Retention time=1.78 min; ESI-MS(+) m/z [M+2H]²⁺: 970.2.

### Preparation of Compound 1223

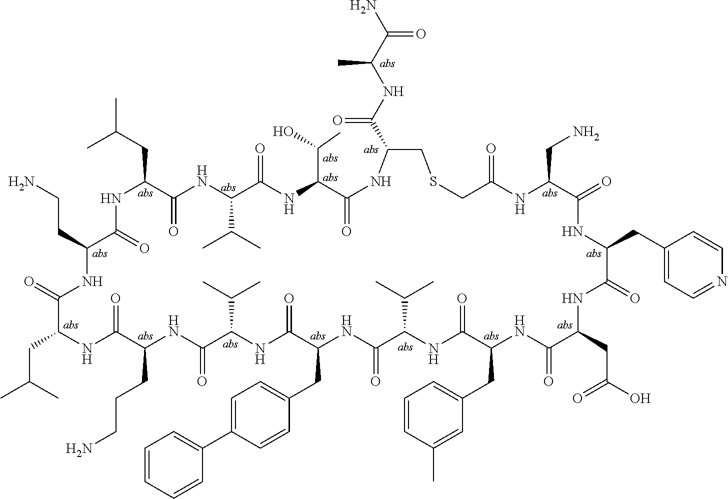 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1223 was prepared on a 50 μmol scale. The yield of the product was 1.3 mg, and its estimated purity by LCMS analysis was 89.7%. Analysis condition B: Retention time=1.35 min; ESI-MS(+) m/z [M+H]⁺: 1804.3.

### Preparation of Compound 1224

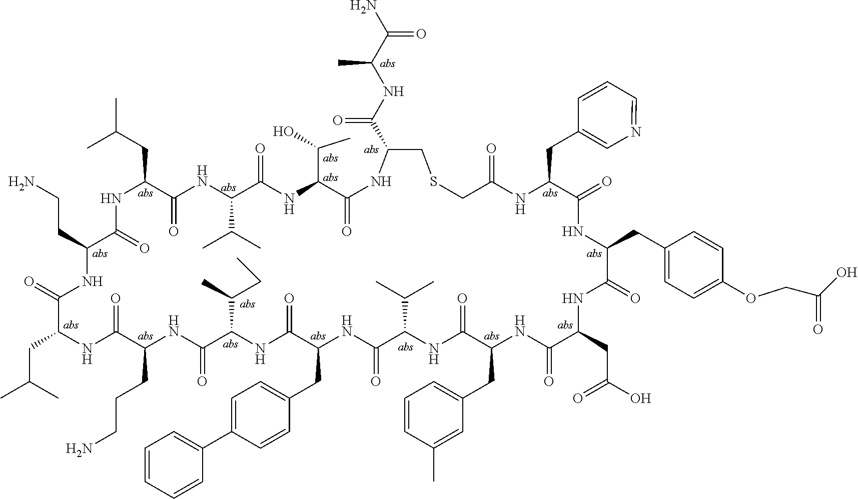 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1224 was prepared on a 50 μmol scale. The yield of the product was 1.4 mg, and its estimated purity by LCMS analysis was 98.5%. Analysis condition A: Retention time=1.6 min; ESI-MS(+) m/z [M+2H]²⁺: 977.14. Analysis condition B: Retention time=1.39 min; ESI-MS(+) m/z [M+2H]²⁺: 977.21.

### Preparation of Compound 1225

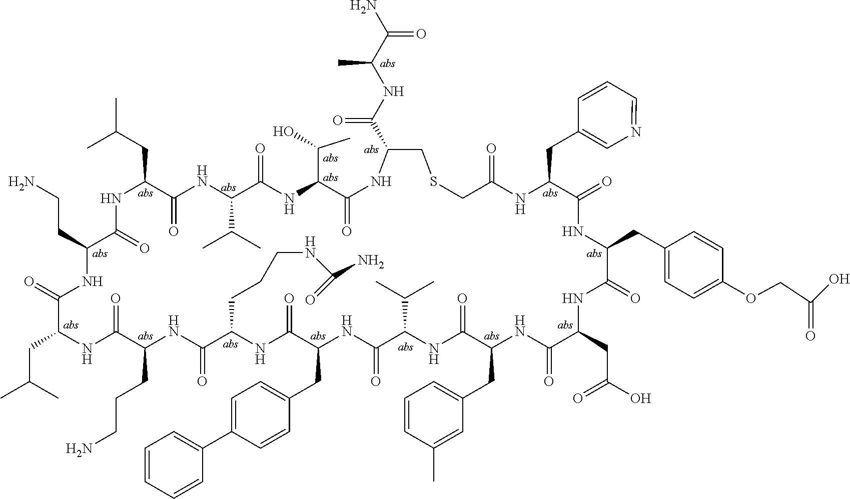

Compound 1225 was prepared on a 50 μmol scale. The yield of the product was 9.4 mg, and its estimated purity by LCMS analysis was 90.6%. Analysis condition B: Retention time=1.41 min; ESI-MS(+) m/z [M+H]: 1997.1.

### Preparation of Compound 1226

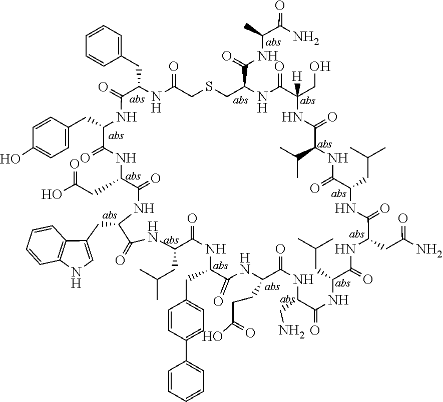 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CN)NC(=O)[C@H](CCC(=O)O)NC(=O)[C@H](Cc2ccc(-c3ccccc3)cc2)NC1=O]

Compound 1226 was prepared on a 50 mol scale. The yield of the product was 7.9 mg, and its estimated purity by LCMS analysis was 94.1%. Analysis condition A: Retention time=1.63 min; ESI-MS(+) m/z [M+H]⁺: 1920.9.

### Preparation of Compound 1227

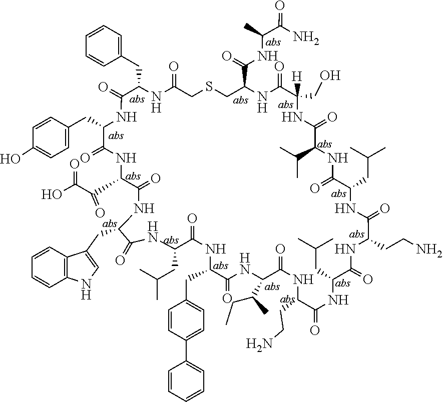 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](C(=O)C(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC1=O]

Compound 1227 was prepared on a 50 μmol scale. The yield of the product was 8.3 mg, and its estimated purity by LCMS analysis was 82.1%. Analysis condition A: Retention time=1.8 min; ESI-MS(+) m/z [M+H]⁺: 1905.1.

### Preparation of Compound 1228

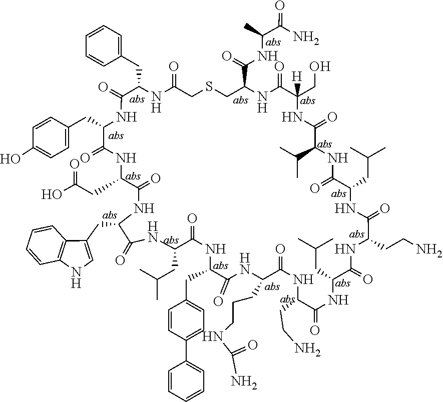 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](CCCNC(N)=O)NC(=O)[C@H](Cc2ccc(-c3ccccc3)cc2)NC1=O]

Compound 1228 was prepared on a 50 mol scale. The yield of the product was 5.7 mg, and its estimated purity by LCMS analysis was 94.5%. Analysis condition A: Retention time=1.63 min; ESI-MS(+) m/z [M+2H]²⁺: 975.2.

### Preparation of Compound 1229

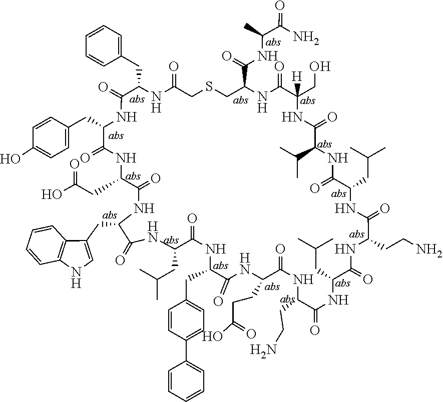 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCN)NC(=O)[C@H](CCC(=O)O)NC(=O)[C@H](Cc2ccc(-c3ccccc3)cc2)NC1=O]

Compound 1229 was prepared on a 50 μmol scale. The yield of the product was 8 mg, and its estimated purity by LCMS analysis was 92.6%. Analysis condition A: Retention time=1.61 min; ESI-MS(+) m/z [M+H]⁺: 1921.

### Preparation of Compound 1230

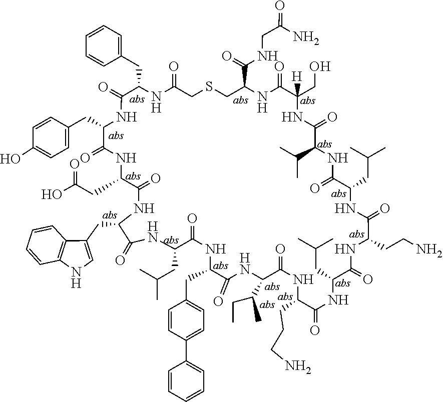 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1230 was prepared on a 50 μmol scale. The yield of the product was 9.6 mg, and its estimated purity by LCMS analysis was 92.8%. Analysis condition B: Retention time=1.68 min; ESI-MS(+) m/z [M+H]⁺: 1904.6.

### Preparation of Compound 1231

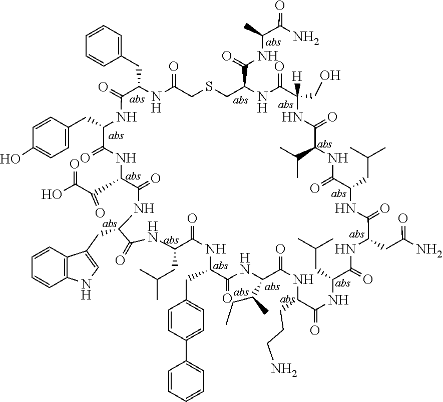 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](C(=O)C(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC1=O]

Compound 1233 was prepared on a 50 μmol scale. The yield of the product was 13.1 mg, and its estimated purity by LCMS analysis was 97.2%. Analysis condition A: Retention time=1.93 min; ESI-MS(+) m/z [M+2H]²⁺: 967.2.

### Preparation of Compound 1234

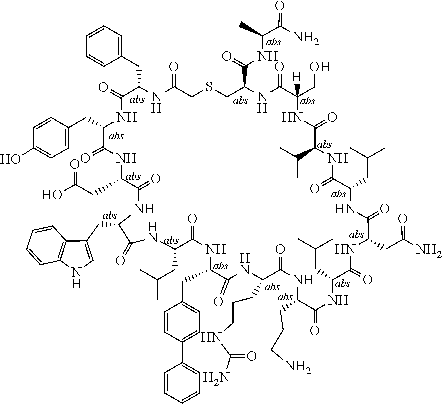 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 1234 was prepared on a 50 μmol scale. The yield of the product was 14.8 mg, and its estimated purity by LCMS analysis was 95%. Analysis condition B: Retention time=1.72 min; ESI-MS(+) m/z [M+H]⁺: 1978.1.

### Preparation of Compound 1235

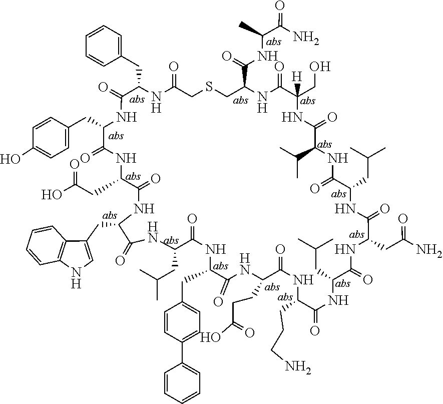 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC1=O]

Compound 1235 was prepared on a 50 μmol scale. The yield of the product was 12.1 mg, and its estimated purity by LCMS analysis was 98.4%. Analysis condition B: Retention time=1.75 min; ESI-MS(+) m/z [M+H]⁺: 1948.9.

### Preparation of Compound 1236

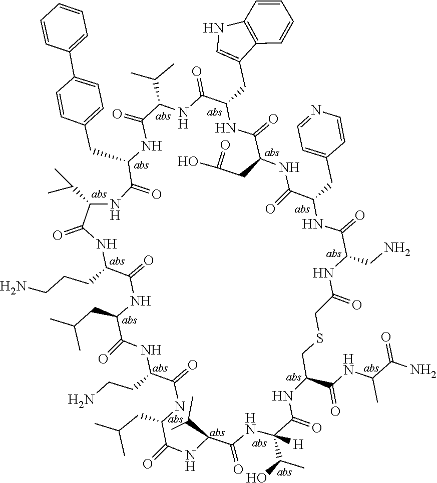

Compound 1236 was prepared on a 50 μmol scale. The yield of the product was 3.5 mg, and its estimated purity by LCMS analysis was 91.1%. Analysis condition B: Retention time=1.22 min; ESI-MS(+) m/z [M+3H]³⁺: 610.6.

### Preparation of Compound 1237

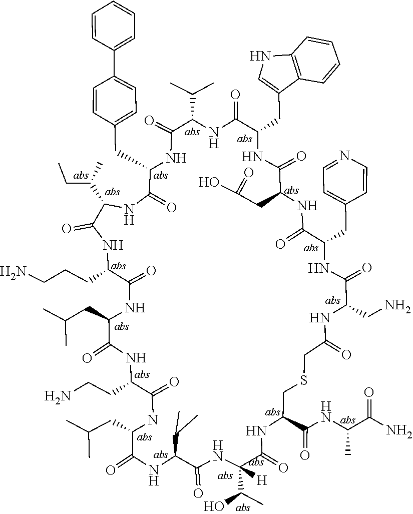

Compound 1237 was prepared on a 50 μmol scale. The yield of the product was 1.4 mg, and its estimated purity by LCMS analysis was 91.1%. Analysis condition A: Retention time=1.45 min; ESI-MS(+) m/z [M+2H]²⁺: 910.1.

### Preparation of Compound 1238

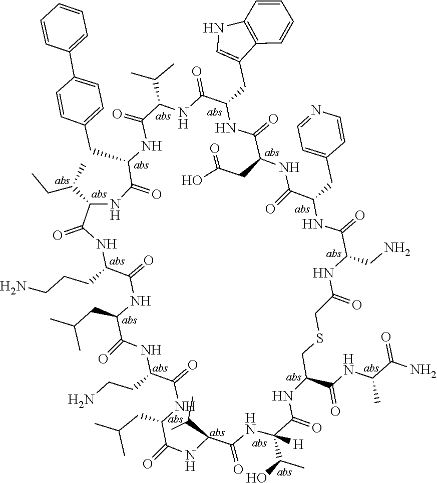 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1238 was prepared on a 50 μmol scale. The yield of the product was 5.2 mg, and its estimated purity by LCMS analysis was 100%. Analysis condition A: Retention time=1.6 min; ESI-MS(+) m/z [M+H]⁺: 1844.1.

### Preparation of Compound 1239

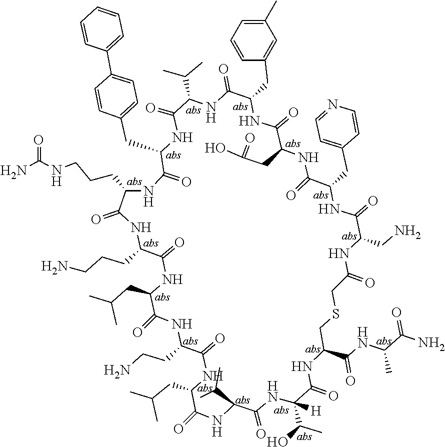 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1239 was prepared on a 50 μmol scale. The yield of the product was 15.4 mg, and its estimated purity by LCMS analysis was 91.4%. Analysis condition B: Retention time=1.22, 1.32 min; ESI-MS(+) m/z [M+3H]³⁺: 621.51, 1862.2.

### Preparation of Compound 1240

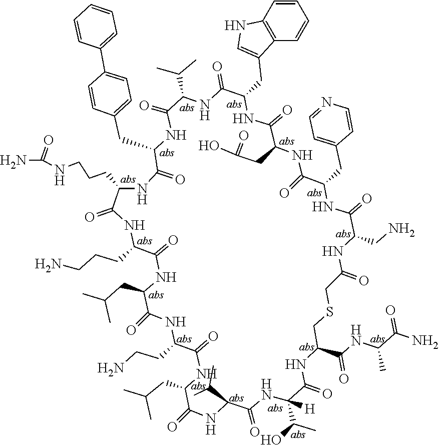 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1240 was prepared on a 50 μmol scale. The yield of the product was 23.4 mg, and its estimated purity by LCMS analysis was 91.2%. Analysis condition A: Retention time=1.4 min; ESI-MS(+) m/z [M+H]⁺: 1886.9.

### Preparation of Compound 1241

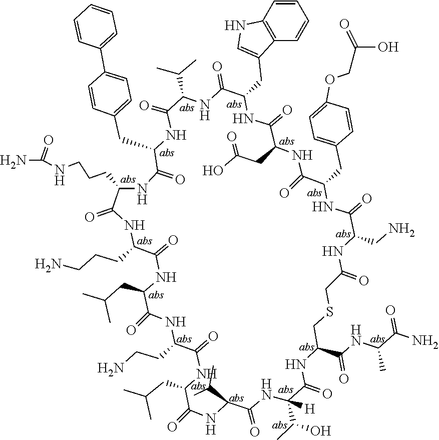 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1241 was prepared on a 50 μmol scale. The yield of the product was 52.2 mg, and its estimated purity by LCMS analysis was 97.9%. Analysis condition B: Retention time=1.21 min; ESI-MS(+) m/z [M+2H]²⁺: 980.3.

### Preparation of Compound 1242

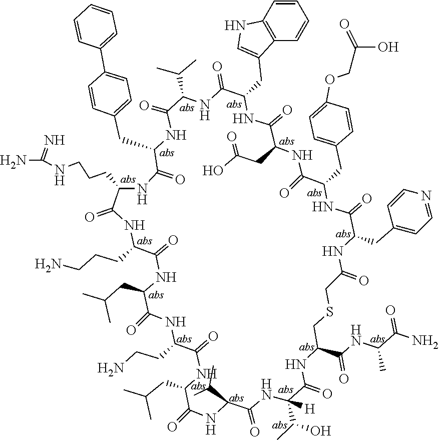 [SMILES: CC(C)C[C@@H]1NC(=O)[C@H](CCN)NC(=O)[C@@H](CC(C)C)NC(=O)[C@H](CCCN)NC(=O)[C@H](CCCNC(=N)N)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC1=O]

Compound 1242 was prepared on a 50 μmol scale. The yield of the product was 20.1 mg, and its estimated purity by LCMS analysis was 94.6%. Analysis condition B: Retention time=1.22 min; ESI-MS(+) m/z [M+3H]³⁺: 674.4.

### Preparation of Compound 1243

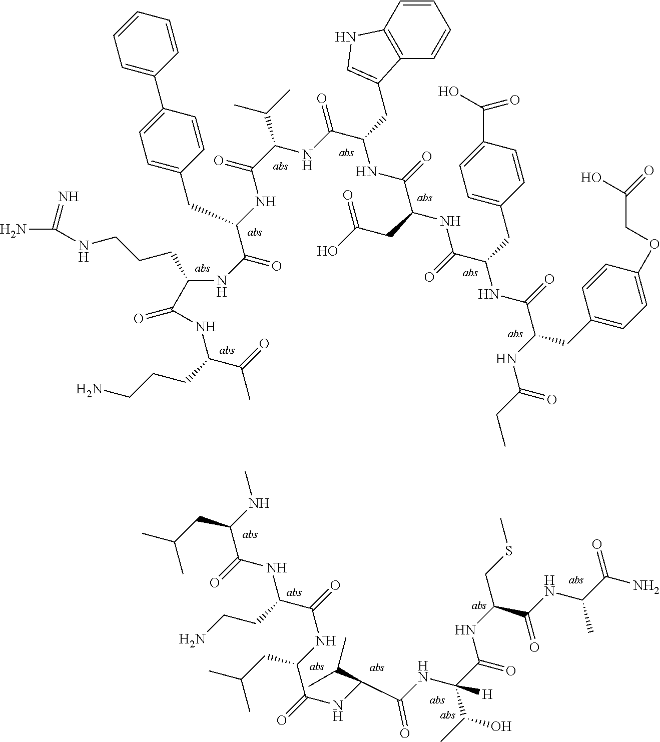 [SMILES: CCC(=O)N[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(=N)N)C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1243 was prepared on a 50 μmol scale. The yield of the product was 61.4 mg, and its estimated purity by LCMS analysis was 96.3%. Analysis condition A: Retention time=1.39 min; ESI-MS(+) m/z [M+2H]²⁺: 1033.2.

### Preparation of Compound 1244

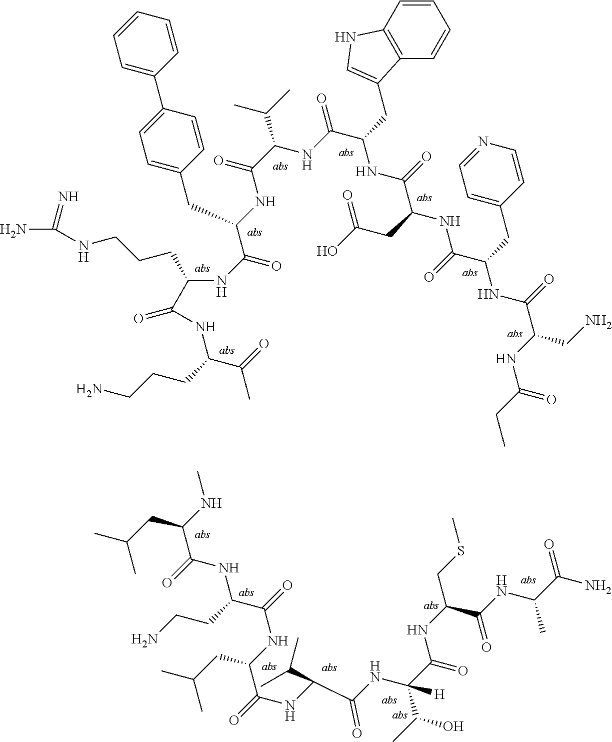 [SMILES: CCC(=O)N[C@@H](CN)C(=O)N[C@@H](Cc1ccncc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(=N)N)C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1244 was prepared on a 50 μmol scale. The yield of the product was 37.6 mg, and its estimated purity by LCMS analysis was 99%. Analysis condition B: Retention time=1.13 min; ESI-MS(+) m/z [M+2H]²⁺: 944.2.

### Preparation of Compound 1245

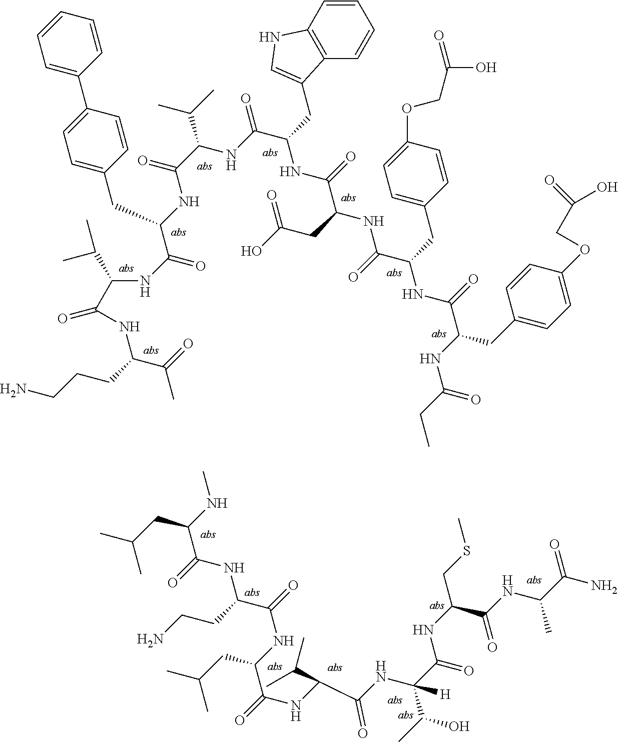 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@H](C(=O)N[C@@H](CCCN)C(C)=O)C(C)C)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1245 was prepared on a 50 μmol scale. The yield of the product was 37.2 mg, and its estimated purity by LCMS analysis was 94.3%. Analysis condition A: Retention time=1.47 min; ESI-MS(+) m/z [M+2H]²⁺: 1020.1.

### Preparation of Compound 1246

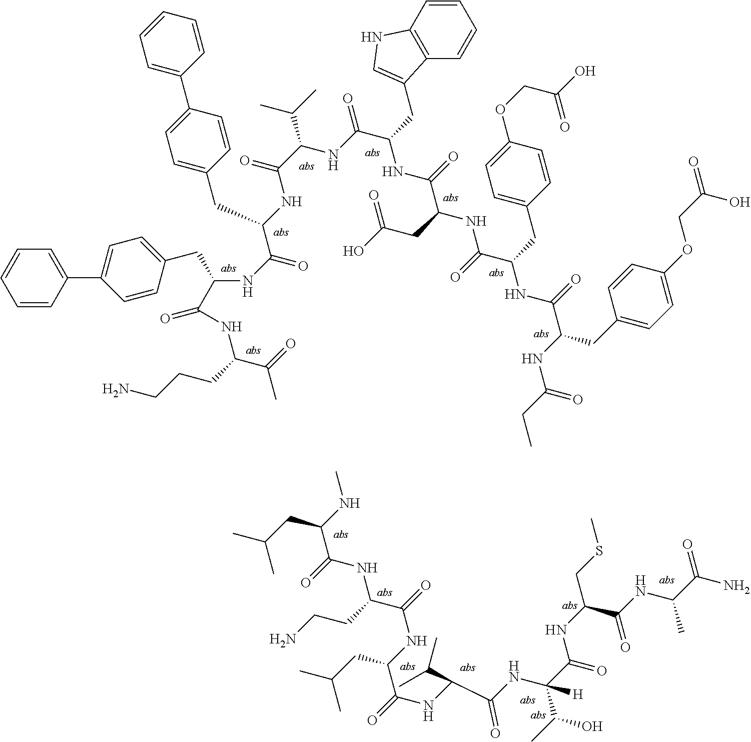 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](Cc1ccc(-c2ccccc2)cc1)C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1246 was prepared on a 50 μmol scale. The yield of the product was 29.6 mg, and its estimated purity by LCMS analysis was 98.5%. Analysis condition A: Retention time=1.79 min; ESI-MS(+) m/z [M+2H]²⁺: 1081.3.

### Preparation of Compound 1247

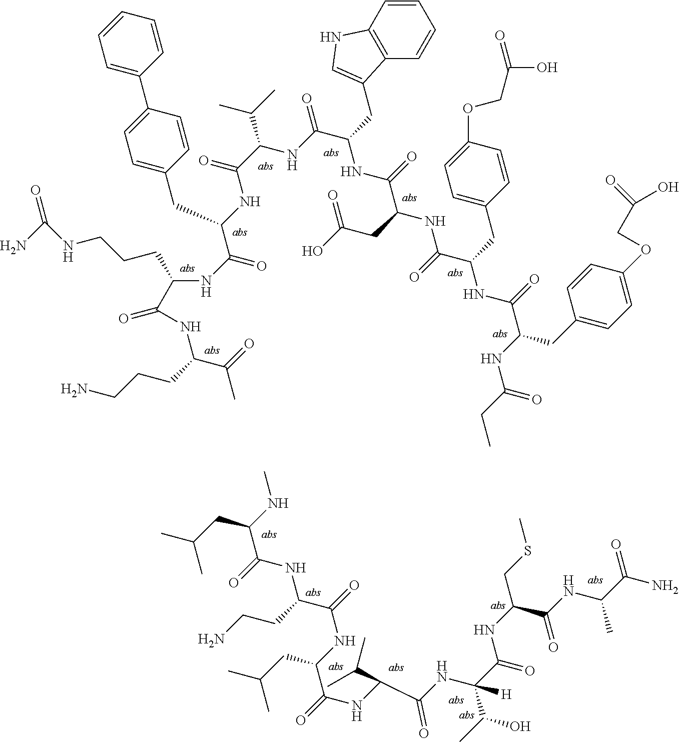 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1247 was prepared on a 50 μmol scale. The yield of the product was 43.3 mg, and its estimated purity by LCMS analysis was 97.9%. Analysis condition B: Retention time=1.53 min; ESI-MS(+) m/z [M+2H]²⁺: 1048.1.

### Preparation of Compound 1248

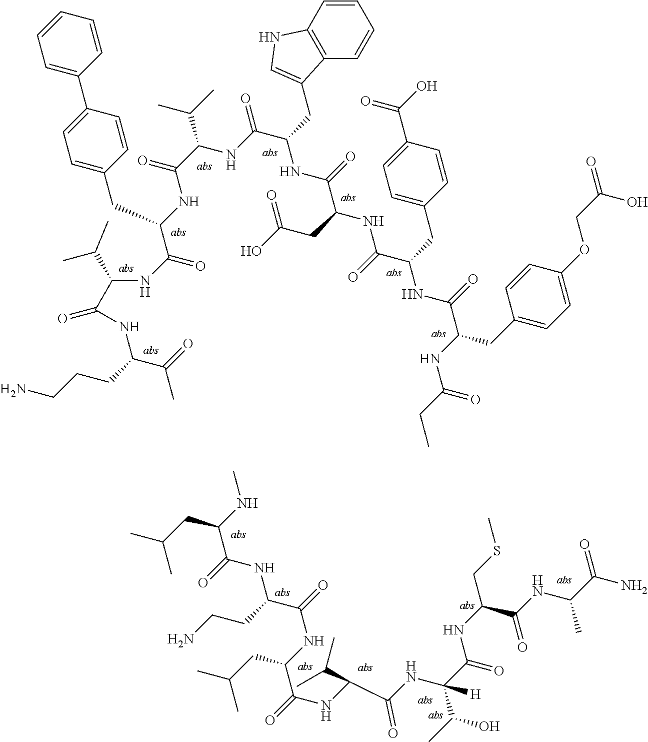 [SMILES: CCC(=O)N[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@H](C(=O)N[C@@H](CCCN)C(C)=O)C(C)C)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1248 was prepared on a 50 μmol scale. The yield of the product was 8.6 mg, and its estimated purity by LCMS analysis was 100%. Analysis condition A: Retention time=1.6 min; ESI-MS(+) m/z [M+2H]²⁺: 1004.

### Preparation of Compound 1249

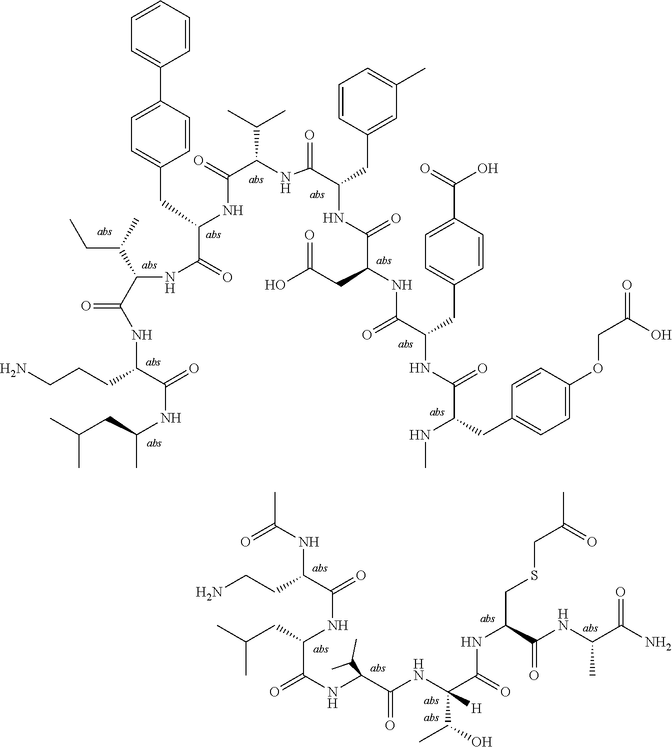 [SMILES: CC[C@H](C)[C@H](NC(=O)[C@H](Cc1ccc(-c2ccccc2)cc1)NC(=O)[C@@H](NC(=O)[C@H](Cc1cccc(C)c1)NC(=O)[C@H](CC(=O)O)NC(=O)[C@H](Cc1ccc(C(=O)O)cc1)NC(=O)[C@H](Cc1ccc(OCC(=O)O)cc1)NC)C(C)C)C(=O)N[C@@H](CCCN)C(=O)N[C@@H](C)CC(C)C ; CC(=O)CSC[C@H](NC(=O)[C@@H](NC(=O)[C@@H](NC(=O)[C@H](CC(C)C)NC(=O)[C@H](CCN)NC(C)=O)C(C)C)[C@@H](C)O)C(=O)N[C@@H](C)C(N)=O]

Compound 1249 was prepared on a 50 μmol scale. The yield of the product was 14.2 mg, and its estimated purity by LCMS analysis was 95.1%. Analysis condition A: Retention time=1.86 min; ESI-MS(+) m/z [M+H]⁺: 1995.8.

### Preparation of Compound 1250

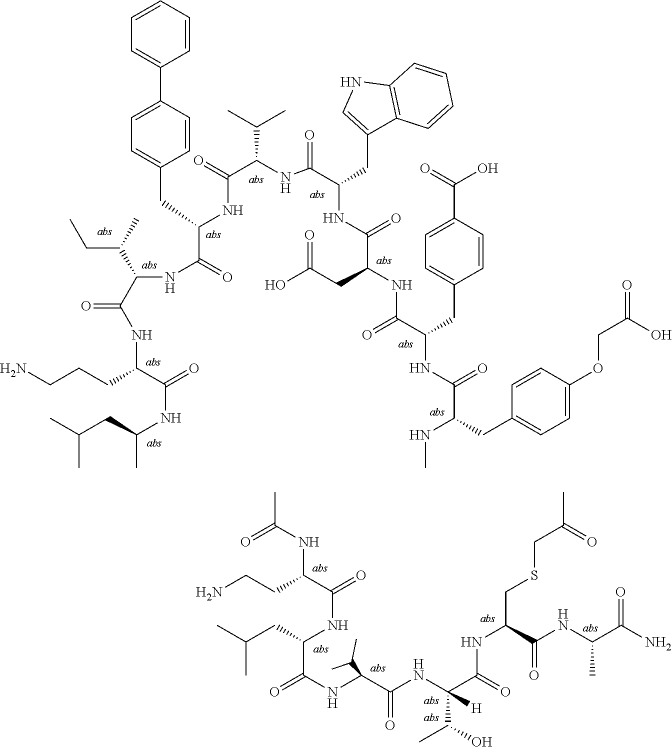 [SMILES: CC[C@H](C)[C@H](NC(=O)[C@H](Cc1ccc(-c2ccccc2)cc1)NC(=O)[C@@H](NC(=O)[C@H](Cc1c[nH]c2ccccc12)NC(=O)[C@H](CC(=O)O)NC(=O)[C@H](Cc1ccc(C(=O)O)cc1)NC(=O)[C@H](Cc1ccc(OCC(=O)O)cc1)NC)C(C)C)C(=O)N[C@@H](CCCN)C(=O)N[C@@H](C)CC(C)C ; CC(=O)CSC[C@H](NC(=O)[C@@H](NC(=O)[C@@H](NC(=O)[C@H](CC(C)C)NC(=O)[C@H](CCN)NC(C)=O)C(C)C)[C@@H](C)O)C(=O)N[C@@H](C)C(N)=O]

Compound 1250 was prepared on a 50 μmol scale. The yield of the product was 15 mg, and its estimated purity by LCMS analysis was 92.9%. Analysis condition B: Retention time=1.48 min; ESI-MS(+) m/z [M+2H]²⁺: 1011.2.

### Preparation of Compound 1251

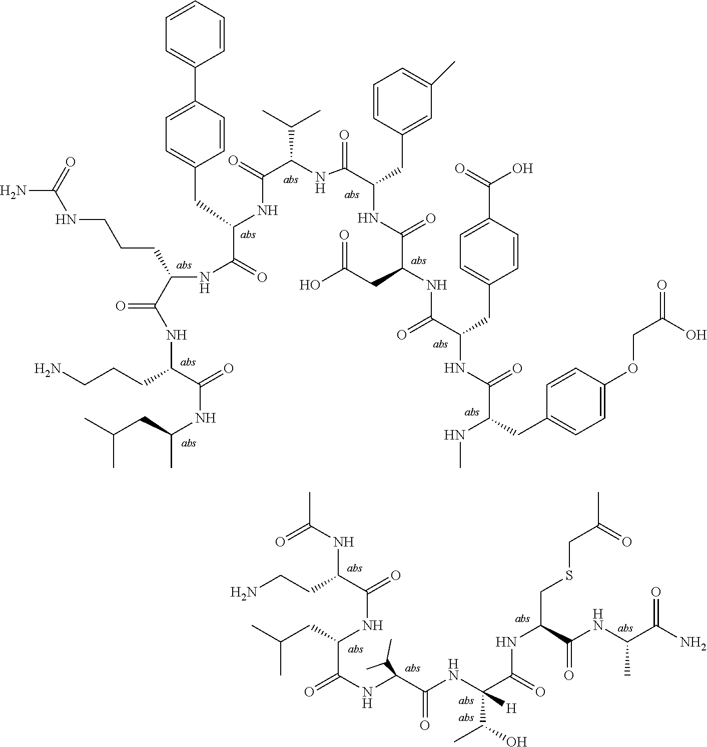 [SMILES: CN[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1cccc(C)c1)C(=O)N[C@H](C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C)C(C)C ; CC(=O)CSC[C@H](NC(=O)[C@@H](NC(=O)[C@@H](NC(=O)[C@H](CC(C)C)NC(=O)[C@H](CCN)NC(C)=O)C(C)C)[C@@H](C)O)C(=O)N[C@@H](C)C(N)=O]

Compound 1251 was prepared on a 50 μmol scale. The yield of the product was 15.4 mg, and its estimated purity by LCMS analysis was 91.7%. Analysis condition A: Retention time=1.49 min; ESI-MS(+) m/z [M+2H]²⁺: 1020.4.

### Preparation of Compound 1252

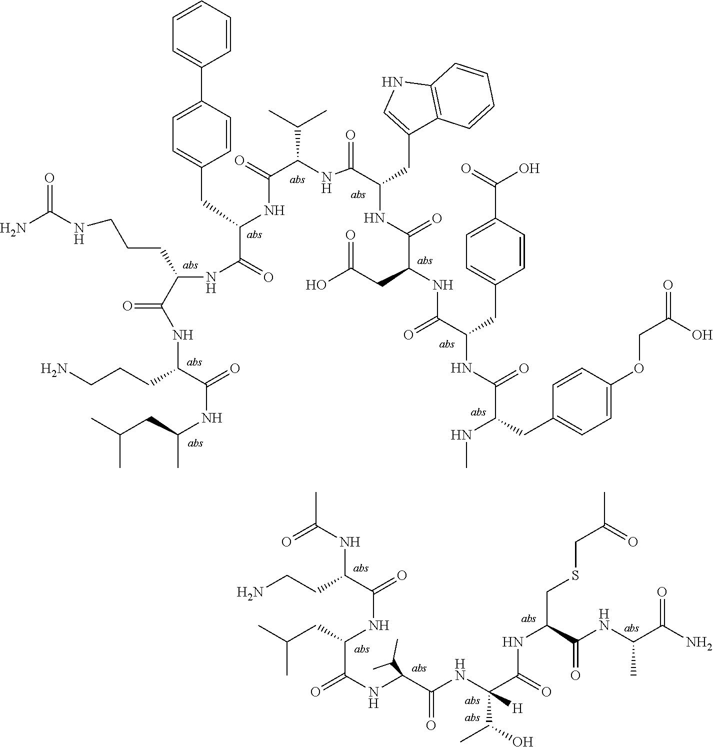 [SMILES: CN[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C)C(C)C ; CC(=O)CSC[C@H](NC(=O)[C@@H](NC(=O)[C@@H](NC(=O)[C@H](CC(C)C)NC(=O)[C@H](CCN)NC(C)=O)C(C)C)[C@@H](C)O)C(=O)N[C@@H](C)C(N)=O]

Compound 1252 was prepared on a 50 μmol scale. The yield of the product was 9.2 mg, and its estimated purity by LCMS analysis was 84.2%. Analysis condition B: Retention time=1.42 min; ESI-MS(+) m/z [M+3H]³⁺: 689.

### Preparation of Compound 1253

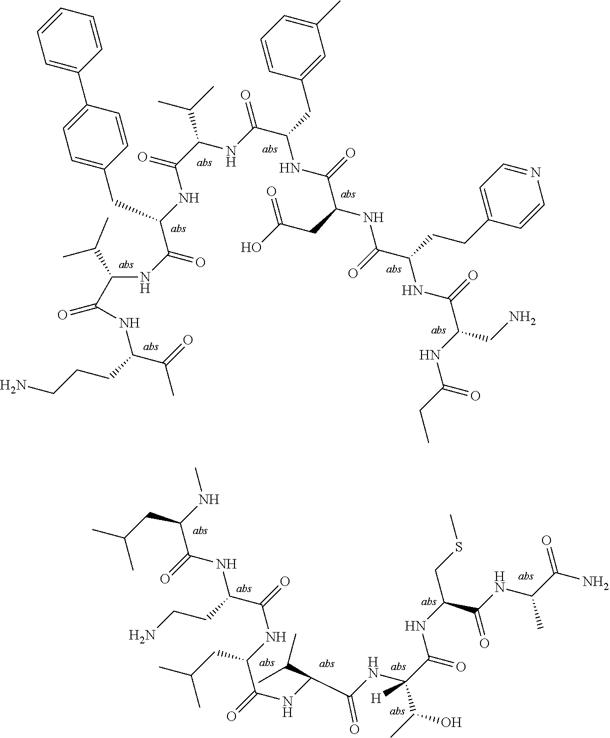 [SMILES: CCC(=O)N[C@@H](CN)C(=O)N[C@@H](CCc1ccncc1)C(=O)N[C@@H](CC(=O)O)C(=O)N[C@@H](Cc1cccc(C)c1)C(=O)N[C@H](C(=O)N[C@@H](Cc1ccc(-c2ccccc2)cc1)C(=O)N[C@H](C(=O)N[C@@H](CCCN)C(C)=O)C(C)C)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@@H](C)O)C(C)C]

Compound 1253 was prepared on a 50 μmol scale. The yield of the product was 3 mg, and its estimated purity by LCMS analysis was 95.8%. Analysis condition A: Retention time=1.4 min; ESI-MS(+) m/z [M+2H]²⁺: 909.9.

### Preparation of Compound 1254

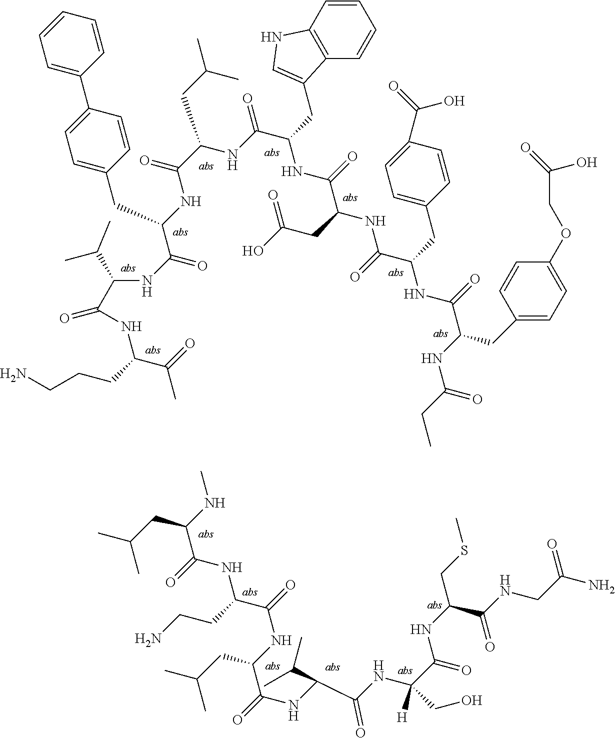 [SMILES: CCC(=O)N[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1254 was prepared on a 50 μmol scale. The yield of the product was 19.4 mg, and its estimated purity by LCMS analysis was 85.8%. Analysis condition A: Retention time=1.6 min; ESI-MS(+) m/z [M+H]⁺: 1993.2.

### Preparation of Compound 1255

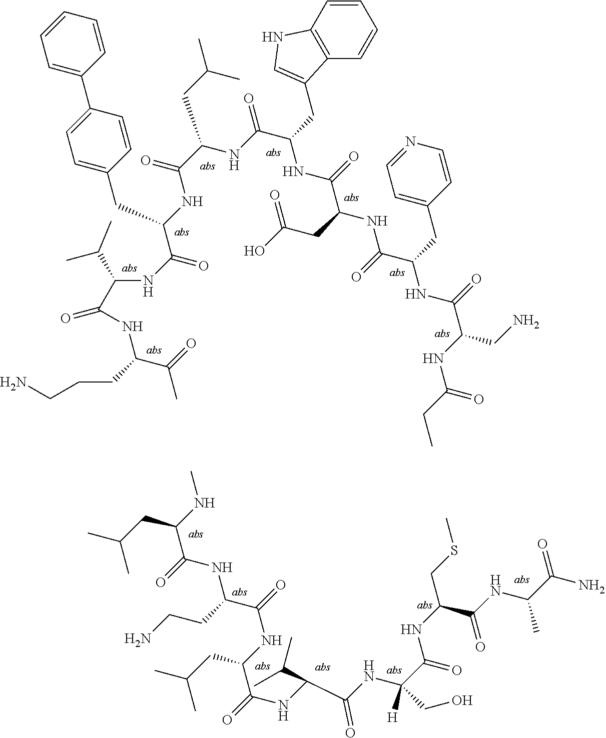 [SMILES: CCC(=O)N[C@@H](CN)C(=O)N[C@@H](Cc1ccncc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)N[C@@H](C)C(N)=O)C(C)C]

Compound 1255 was prepared on a 50 μmol scale. The yield of the product was 26.2 mg, and its estimated purity by LCMS analysis was 85.1%. Analysis condition A: Retention time=1.39 min; ESI-MS(+) m/z [M+2H]²⁺: 908.1.

### Preparation of Compound 1256

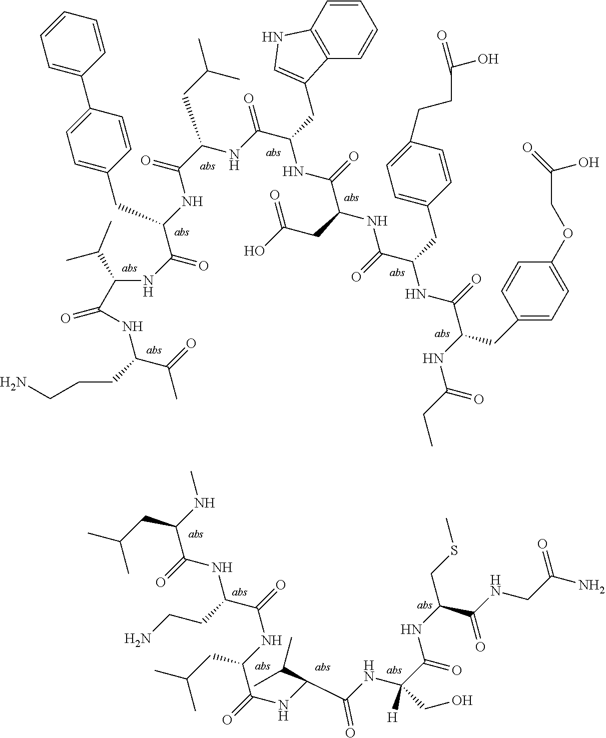 [SMILES: CCC(=O)N[C@@H](Cc1ccc(OCC(=O)O)cc1)C(=O)N[C@@H](Cc1ccc(CCC(=O)O)cc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1256 was prepared on a 50 μmol scale. The yield of the product was 19.4 mg, and its estimated purity by LCMS analysis was 91.8%. Analysis condition A: Retention time=1.51 min; ESI-MS(+) m/z [M+2H]²⁺: 1012.1.

### Preparation of Compound 1257

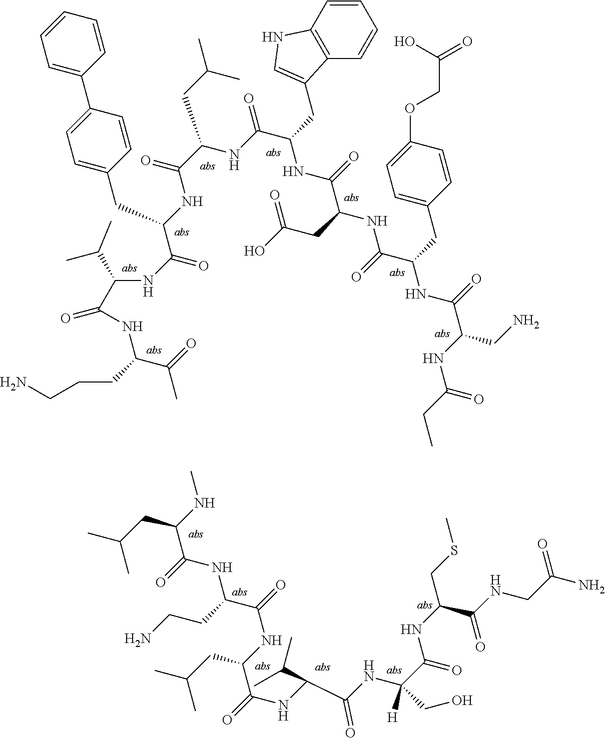 [SMILES: CCC(=O)N[C@@H](CN)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1257 was prepared on a 50 μmol scale. The yield of the product was 13 mg, and its estimated purity by LCMS analysis was 96.4%. Analysis condition A: Retention time=1.6 min; ESI-MS(+) m/z [M+H]⁺: 1888.

### Preparation of Compound 1258

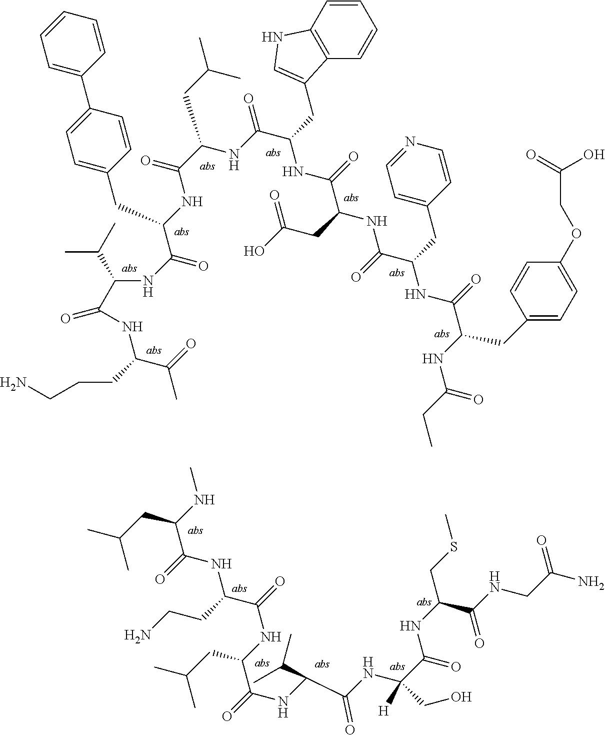 [SMILES: CCC(=O)N[C@@H](Cc1ccc(OCC(=O)O)cc1)C(=O)N[C@@H](Cc1ccncc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1258 was prepared on a 50 μmol scale. The yield of the product was 4.7 mg, and its estimated purity by LCMS analysis was 96.9%. Analysis condition B: Retention time=1.33 min; ESI-MS(+) m/z [M+2H]²⁺: 976.2.

### Preparation of Compound 1259

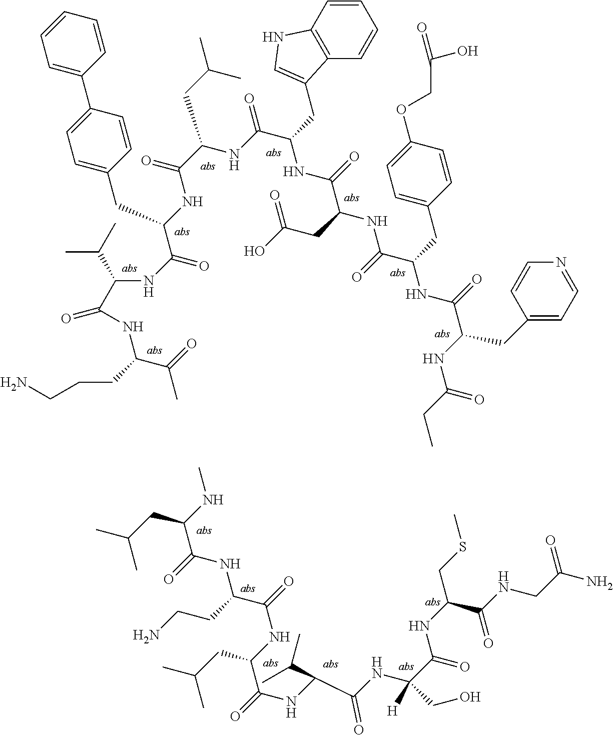 [SMILES: CCC(=O)N[C@@H](Cc1ccncc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1259 was prepared on a 50 μmol scale. The yield of the product was 10.7 mg, and its estimated purity by LCMS analysis was 81.5%. Analysis condition B: Retention time=1.47 min; ESI-MS(+) m/z [M+H]⁺: 1972.8.

### Preparation of Compound 1260

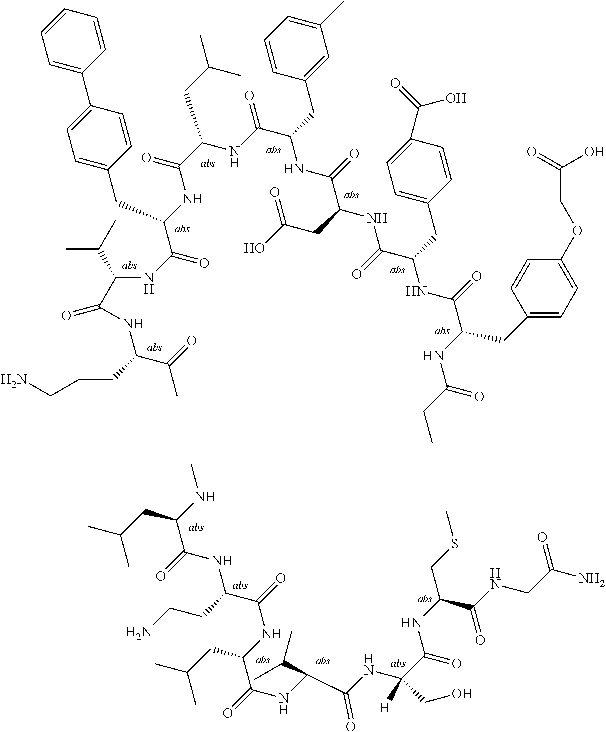 [SMILES: CCC(=O)N[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1260 was prepared on a 50 μmol scale. The yield of the product was 31.4 mg, and its estimated purity by LCMS analysis was 86.8%. Analysis condition A: Retention time=1.63 min; ESI-MS(+) m/z [M+2H]²⁺: 985.2.

### Preparation of Compound 1261

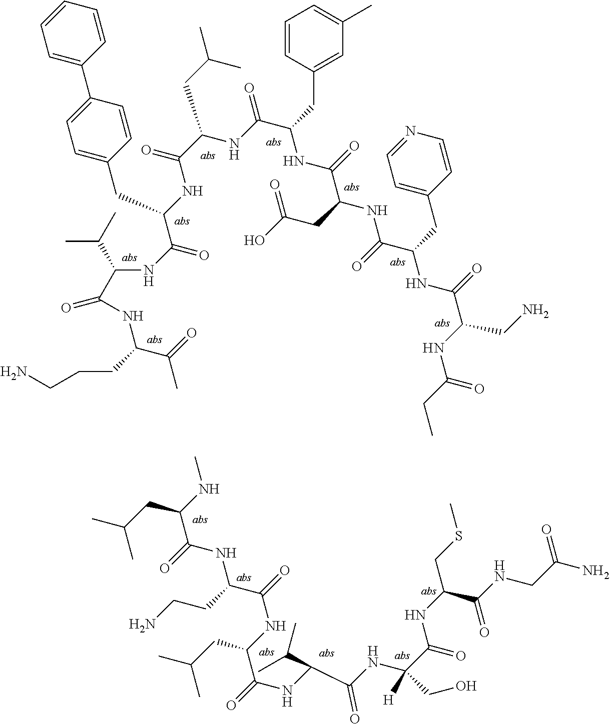 [SMILES: CCC(=O)N[C@@H](CN)C(=O)N[C@@H](Cc1ccncc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1261 was prepared on a 50 μmol scale. The yield of the product was 1.3 mg, and its estimated purity by LCMS analysis was 87.9%. Analysis condition A: Retention time=1.66 min; ESI-MS(+) m/z [M+2H]²⁺: 896.1.

### Preparation of Compound 1262

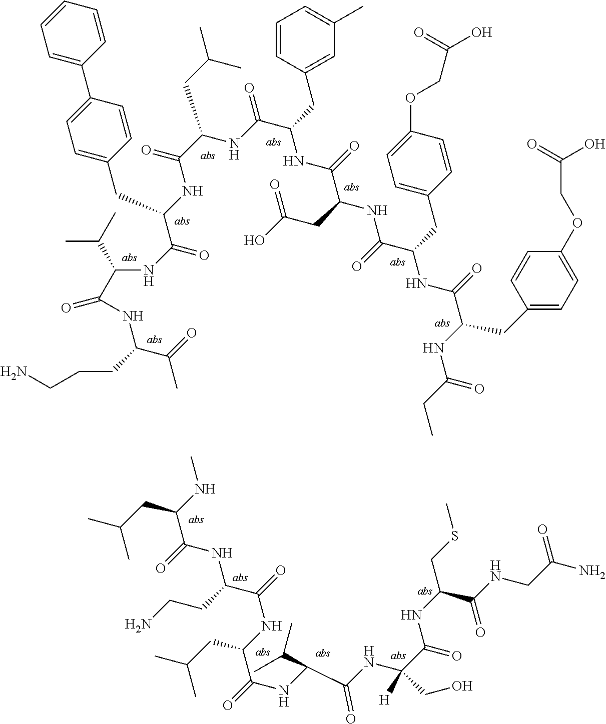 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1262 was prepared on a 50 μmol scale. The yield of the product was 9.4 mg, and its estimated purity by LCMS analysis was 93.1%. Analysis condition A: Retention time=1.6 min; ESI-MS(+) m/z [M+2H]²⁺: 1000.2.

### Preparation of Compound 1263

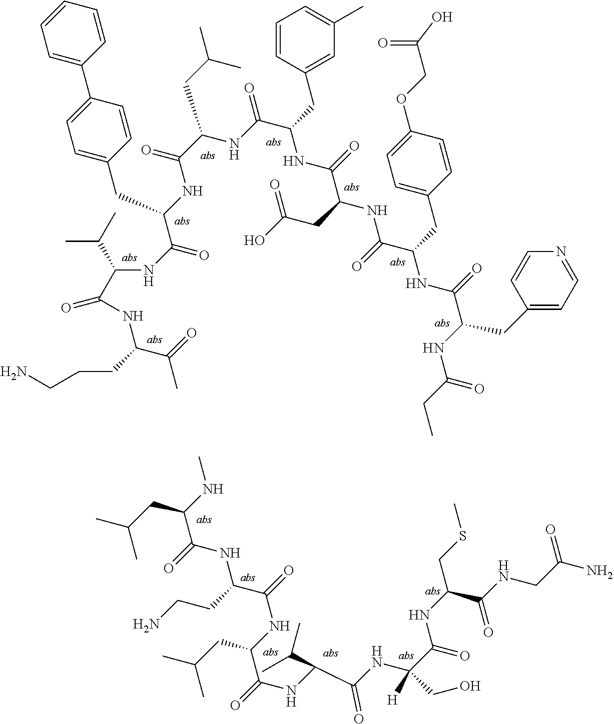 [SMILES: CCC(=O)N[C@@H](Cc1ccncc1)C(=O)N[C@@H](Cc1ccc(OCC(=O)O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1263 was prepared on a 50 μmol scale. The yield of the product was 5.9 mg, and its estimated purity by LCMS analysis was 87%. Analysis condition B: Retention time=1.4 min; ESI-MS(+) m/z [M+2H]²⁺: 963.2.

### Preparation of Compound 1264

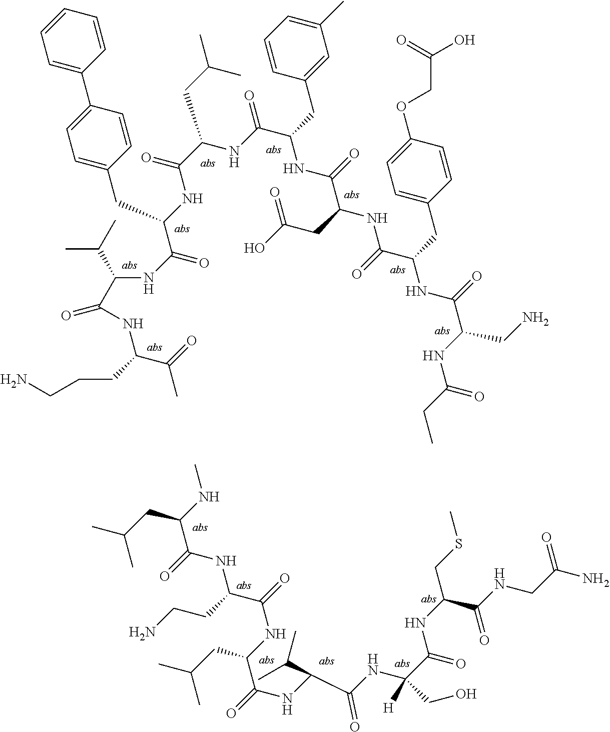 [SMILES: CCC(=O)N[C@@H](CN)C(=O)N[C@@H](Cc1ccc(OCC(=O)O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1264 was prepared on a 50 μmol scale. The yield of the product was 40.6 mg, and its estimated purity by LCMS analysis was 92.1%. Analysis condition A: Retention time=1.63 min; ESI-MS(+) m/z [M+2H]²⁺: 932.2.

### Preparation of Compound 1265

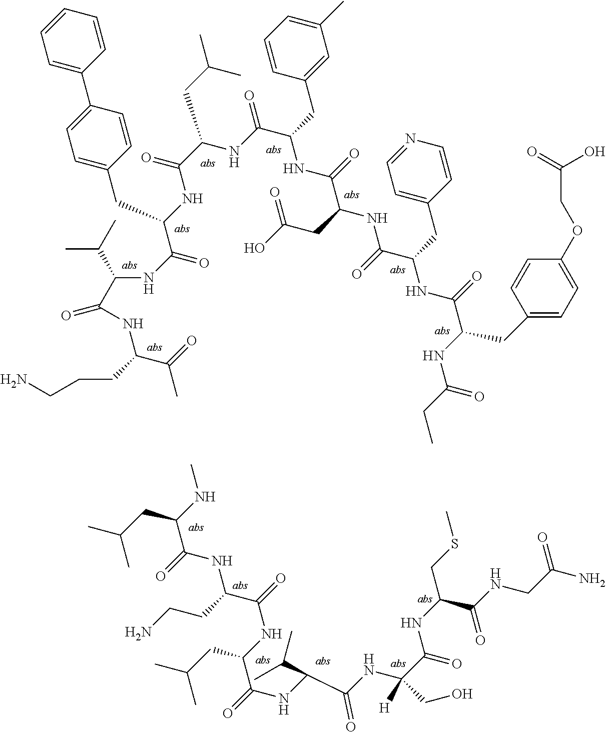 [SMILES: CCC(=O)N[C@@H](Cc1ccc(OCC(=O)O)cc1)C(=O)N[C@@H](Cc1ccncc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@H](C(=O)N[C@@H](CCCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)NCC(N)=O)C(C)C]

Compound 1265 was prepared on a 50 μmol scale. The yield of the product was 9.1 mg, and its estimated purity by LCMS analysis was 94.5%. Analysis condition B: Retention time=1.36 min; ESI-MS(+) m/z [M+2H]²⁺: 963.2.

### Preparation of Compound 1266

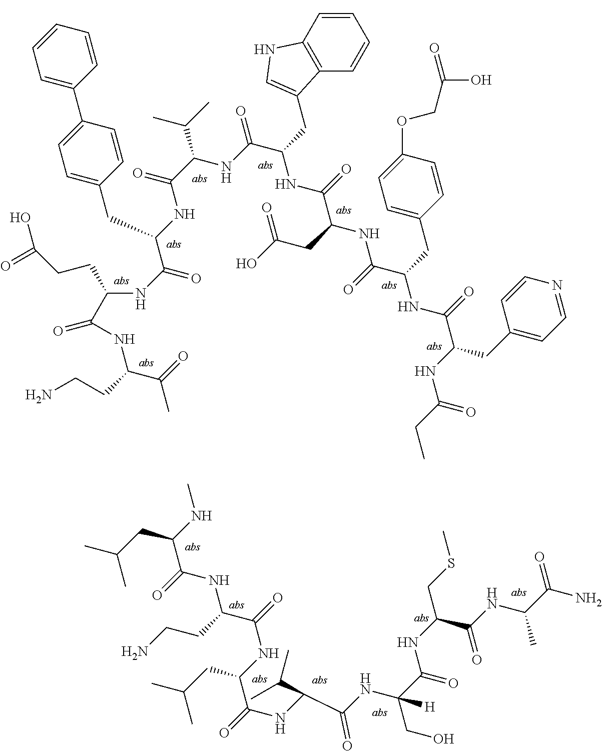 [SMILES: CCC(=O)N[C@@H](Cc1ccncc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCC(=O)O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)N[C@@H](C)C(N)=O)C(C)C]

Compound 1266 was prepared on a 50 μmol scale. The yield of the product was 16.7 mg, and its estimated purity by LCMS analysis was 93.7%. Analysis condition A: Retention time=1.39 min; ESI-MS(+) m/z [M+2H]²⁺: 984.2.

### Preparation of Compound 1267

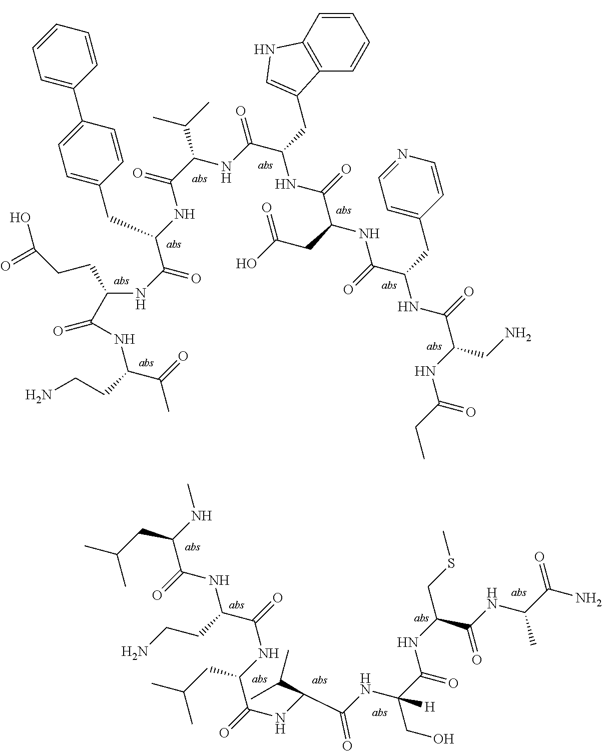 [SMILES: CCC(=O)N[C@@H](CN)C(=O)N[C@@H](Cc1ccncc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCC(=O)O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)N[C@@H](C)C(N)=O)C(C)C]

Compound 1267 was prepared on a 50 μmol scale. The yield of the product was 17.1 mg, and its estimated purity by LCMS analysis was 96.2%. Analysis condition B: Retention time=1.18 min; ESI-MS(+) m/z [M+2H]²⁺: 916.3.

### Preparation of Compound 1268

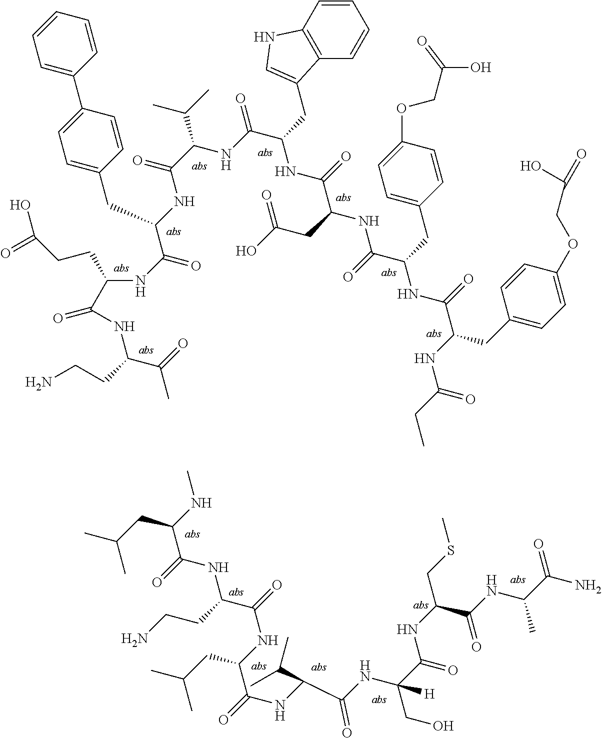 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCC(=O)O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)N[C@@H](C)C(N)=O)C(C)C]

Compound 1268 was prepared on a 50 μmol scale. The yield of the product was 11.4 mg, and its estimated purity by LCMS analysis was 96.2%. Analysis condition A: Retention time=1.36 min; ESI-MS(+) m/z [M+2H]²⁺: 1020.1.

### Preparation of Compound 1269

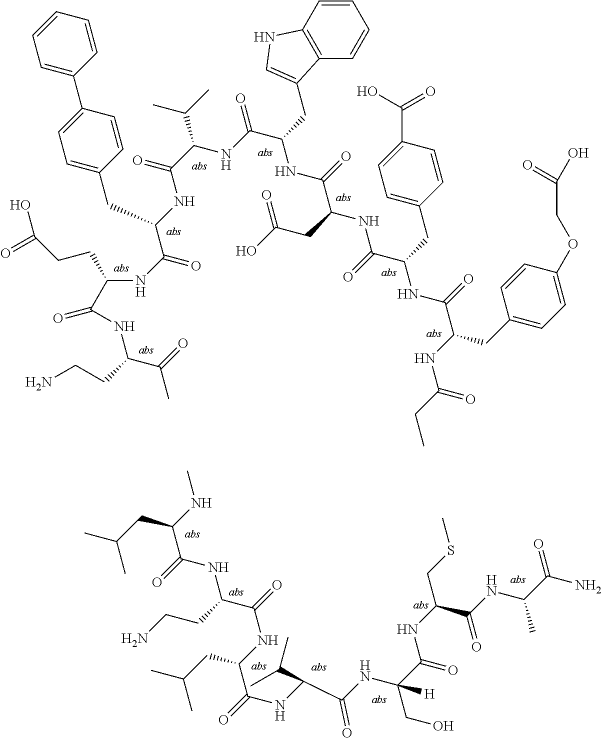 [SMILES: CCC(=O)N[C@@H](Cc1ccc(OCC(=O)O)cc1)C(=O)N[C@@H](Cc1ccc(C(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCC(=O)O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@@H](CO)C(=O)N[C@@H](CSC)C(=O)N[C@@H](C)C(N)=O)C(C)C]

Compound 1269 was prepared on a 50 μmol scale. The yield of the product was 19 mg, and its estimated purity by LCMS analysis was 95%. Analysis condition B: Retention time=1.5 min; ESI-MS(+) m/z [M+2H]²⁺: 1005.4.

### Preparation of Compound 1270

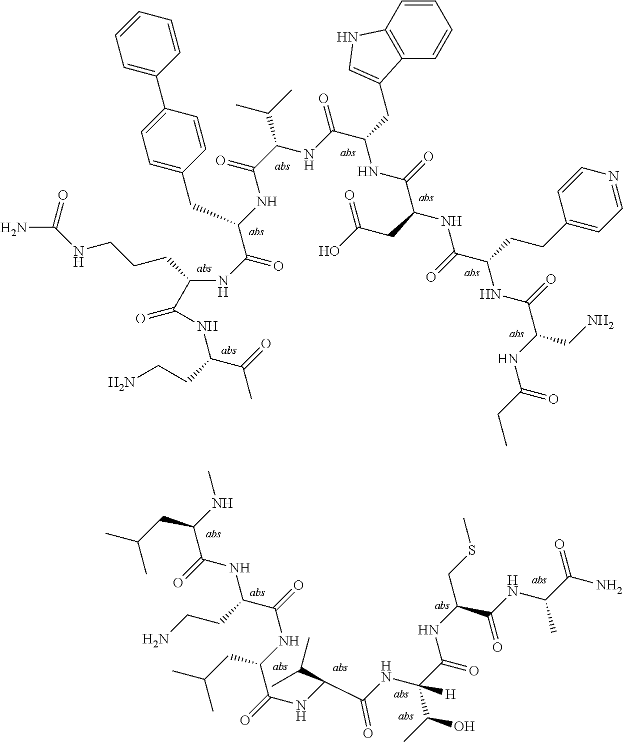 [SMILES: CCC(=O)N[C@@H](CN)C(=O)N[C@@H](CCc1ccncc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@H](C)O)C(C)C]

Compound 1270 was prepared on a 50 μmol scale. The yield of the product was 15.9 mg, and its estimated purity by LCMS analysis was 96.2%. Analysis condition B: Retention time=1.18 min; ESI-MS(+) m/z [M+3H]³⁺: 635.

### Preparation of Compound 1271

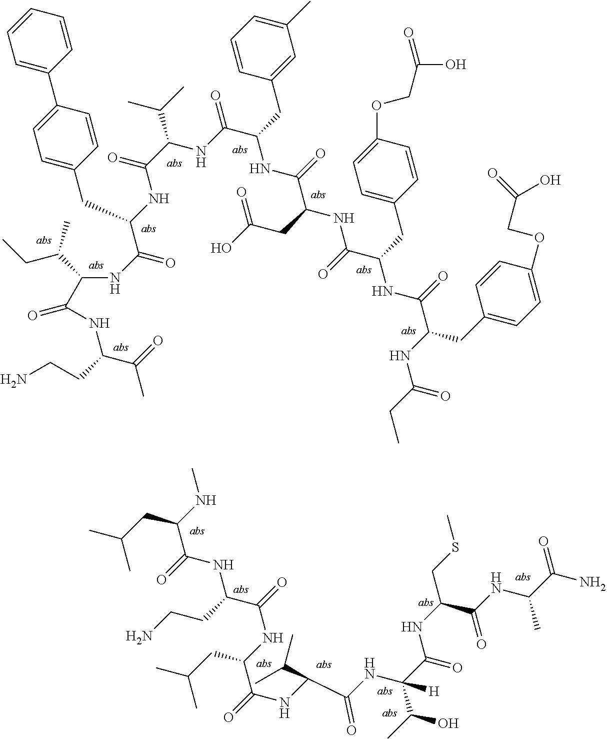 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1cccc(C)c1)C(=O)N[C@H](C(=O)N[C@@H](Cc1ccc(-c2ccccc2)cc1)C(=O)N[C@H](C(=O)N[C@@H](CCN)C(C)=O)[C@@H](C)CC)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@H](C)O)C(C)C]

Compound 1271 was prepared on a 50 μmol scale. The yield of the product was 43.8 mg, and its estimated purity by LCMS analysis was 95.4%. Analysis condition A: Retention time=1.64 min; ESI-MS(+) m/z [M+2H]²⁺: 1014.

### Preparation of Compound 1272

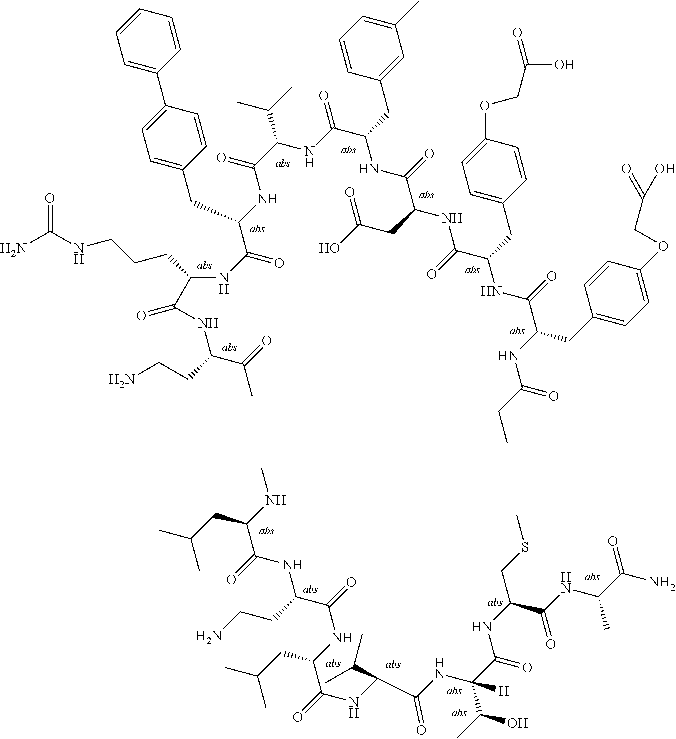 [SMILES: CCC(=O)N[C@@H](Cc1ccc(OCC(=O)O)cc1)C(=O)N[C@@H](Cc1ccc(OCC(=O)O)cc1)C(=O)N[C@@H](CC(=O)O)C(=O)N[C@@H](Cc1cccc(C)c1)C(=O)N[C@H](C(=O)N[C@@H](Cc1ccc(-c2ccccc2)cc1)C(=O)N[C@@H](CCCNC(N)=O)C(=O)N[C@@H](CCN)C(C)=O)C(C)C ; CN[C@H](CC(C)C)C(=O)N[C@@H](CCN)C(=O)N[C@@H](CC(C)C)C(=O)N[C@H](C(=O)N[C@H](C(=O)N[C@@H](CSC)C(=O)N[C@@H](C)C(N)=O)[C@H](C)O)C(C)C]

Compound 1272 was prepared on a 50 μmol scale. The yield of the product was 3.3 mg, and its estimated purity by LCMS analysis was 87.7%. Analysis condition B: Retention time=1.47 min; ESI-MS(+) m/z [M+2H]²⁺: 1036.1.

### Preparation of Compound 1273

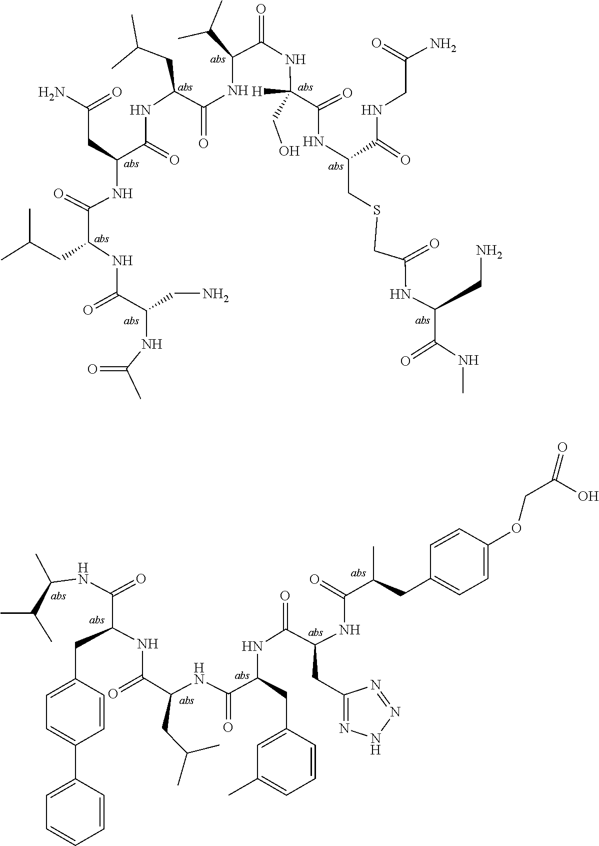 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; Cc1cccc(C[C@H](NC(=O)[C@H](Cc2nn[nH]n2)NC(=O)[C@@H](C)Cc2ccc(OCC(=O)O)cc2)C(=O)N[C@@H](CC(C)C)C(=O)N[C@@H](Cc2ccc(-c3ccccc3)cc2)C(=O)N[C@H](C)C(C)C)c1]

Compound 1273 was prepared on a 50 μmol scale. The yield of the product was 5.2 mg, and its estimated purity by LCMS analysis was 91.2%. Analysis condition B: Retention time=1.59, 1.62 min; ESI-MS(+) m/z [M+2H]²⁺: 937.11, 937.11.

### Preparation of Compound 1274

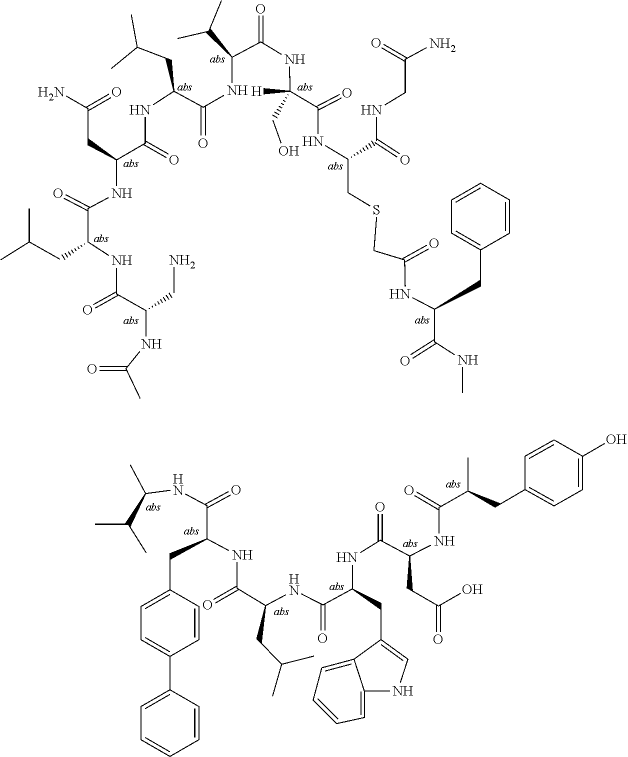 [SMILES: CNC(=O)[C@H](Cc1ccccc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(O)cc1)C(=O)N[C@@H](Cc1ccc(-c2ccccc2)cc1)C(=O)N[C@H](C)C(C)C]

Compound 1274 was prepared on a 50 μmol scale. The yield of the product was 25.6 mg, and its estimated purity by LCMS analysis was 100%. Analysis condition A: Retention time=1.86 min; ESI-MS(+) m/z [M+H]⁺: 1877.

### Preparation of Compound 1275

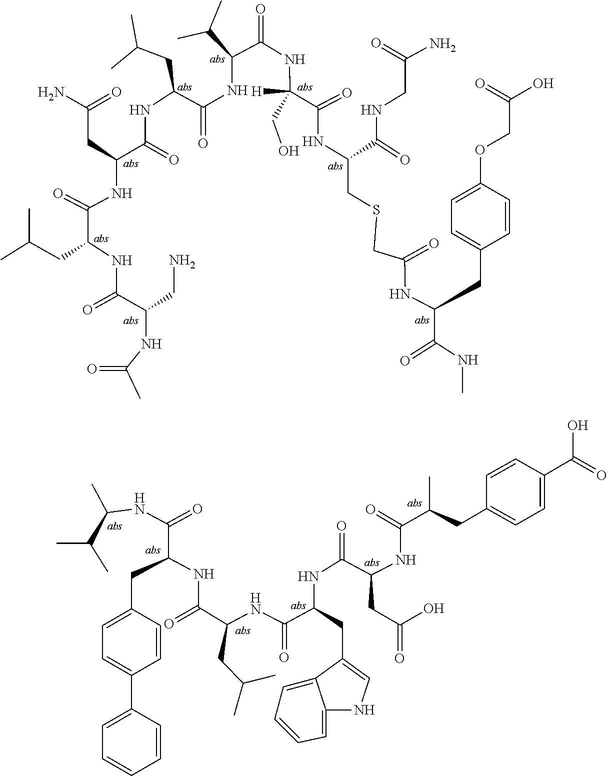 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(C(=O)O)cc1)C(=O)N[C@@H](Cc1ccc(-c2ccccc2)cc1)C(=O)N[C@H](C)C(C)C]

Compound 1275 was prepared on a 50 μmol scale. The yield of the product was 24.3 mg, and its estimated purity by LCMS analysis was 93.2%. Analysis condition A: Retention time=1.46 min; ESI-MS(+) m/z [M+2H]²⁺: 989.9.

### Preparation of Compound 1276

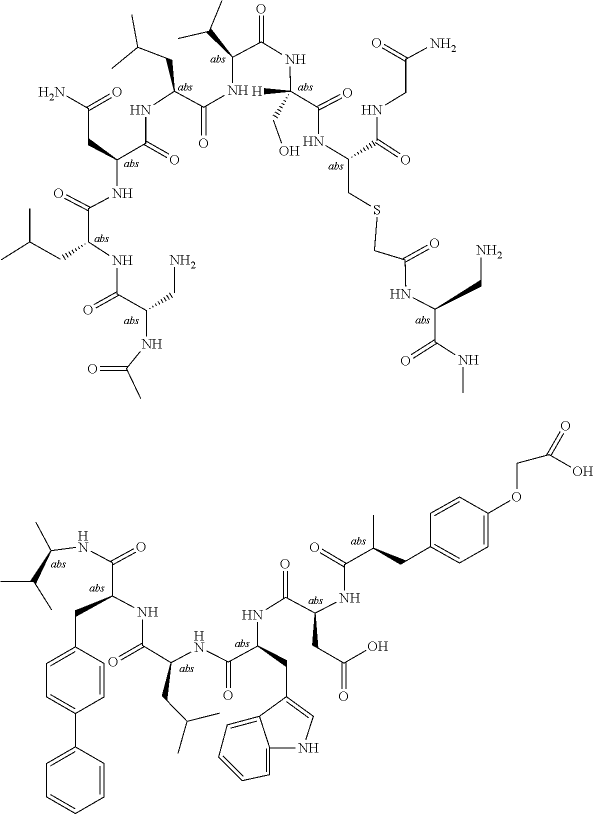 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(OCC(=O)O)cc1)C(=O)N[C@@H](Cc1ccc(-c2ccccc2)cc1)C(=O)N[C@H](C)C(C)C]

Compound 1276 was prepared on a 50 μmol scale. The yield of the product was 19.9 mg, and its estimated purity by LCMS analysis was 97%. Analysis condition A: Retention time=1.72 min; ESI-MS(+) m/z [M+2H]²⁺: 938.1.

### Preparation of Compound 1277

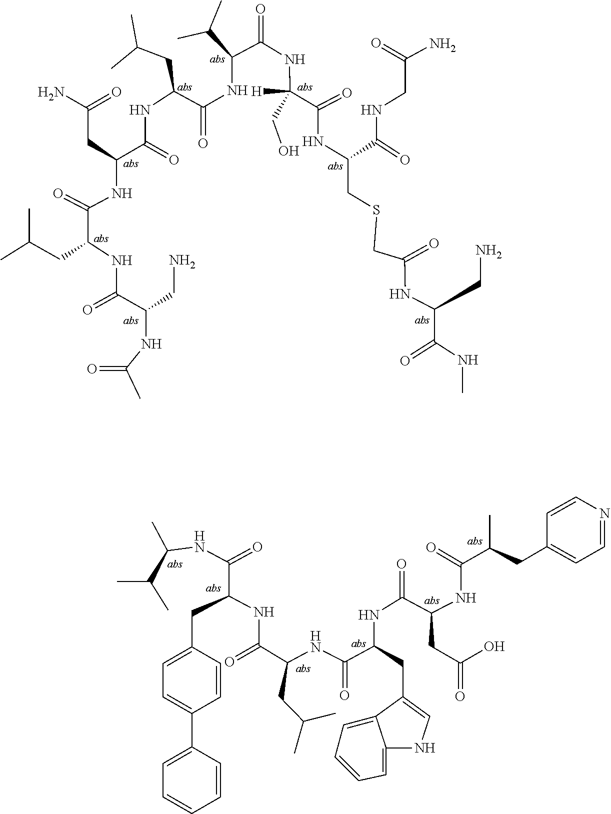 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1277 was prepared on a 50 μmol scale. The yield of the product was 24.8 mg, and its estimated purity by LCMS analysis was 95.5%. Analysis condition A: Retention time=1.79 min; ESI-MS(+) m/z [M+3H]³⁺: 601.2.

### Preparation of Compound 1278

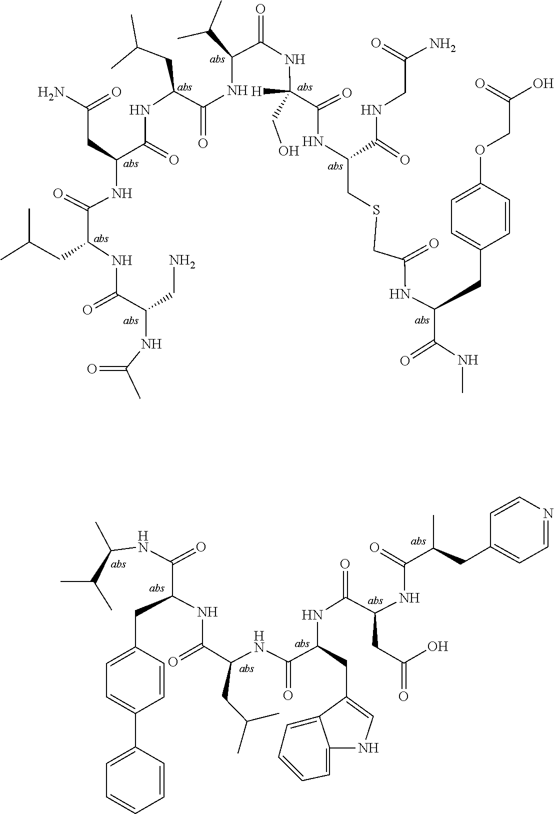 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1278 was prepared on a 50 μmol scale. The yield of the product was 7.2 mg, and its estimated purity by LCMS analysis was 97.4%. Analysis condition A: Retention time=1.61 min; ESI-MS(+) m/z [M+H]⁺: 1936.

### Preparation of Compound 1279

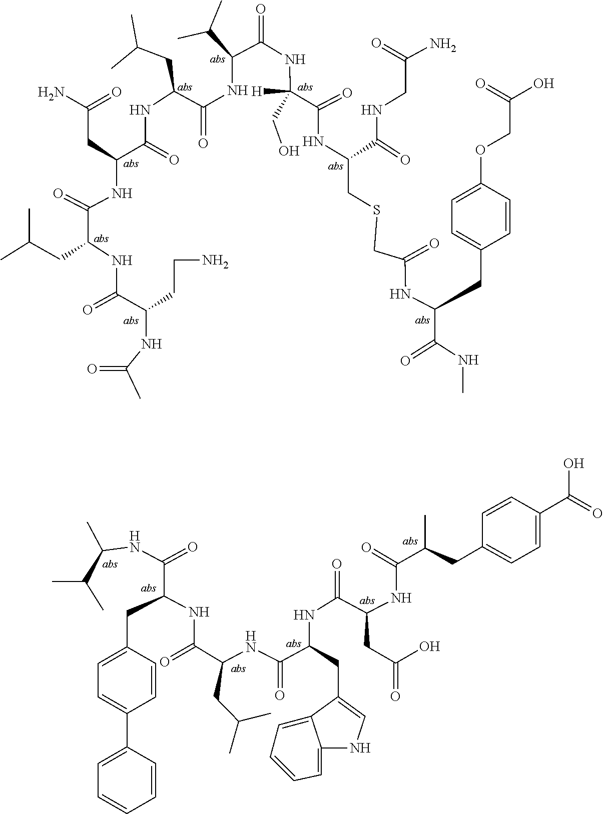 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(C(=O)O)cc1)C(=O)N[C@@H](Cc1ccc(-c2ccccc2)cc1)C(=O)N[C@H](C)C(C)C]

Compound 1279 was prepared on a 50 μmol scale. The yield of the product was 11.4 mg, and its estimated purity by LCMS analysis was 98.2%. Analysis condition B: Retention time=1.71 min; ESI-MS(+) m/z [M+H]⁺: 1993.1.

### Preparation of Compound 1280

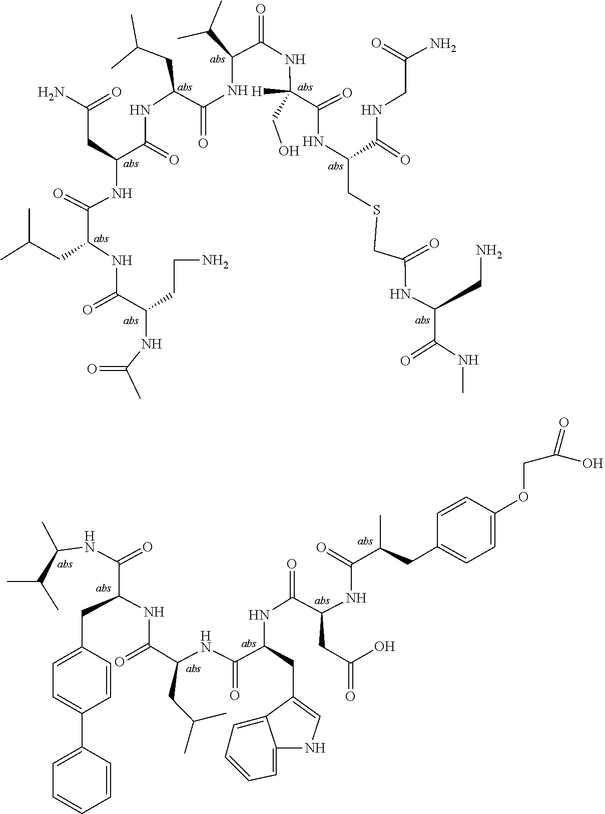 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(OCC(=O)O)cc1)C(=O)N[C@@H](Cc1ccc(-c2ccccc2)cc1)C(=O)N[C@H](C)C(C)C]

Compound 1280 was prepared on a 50 μmol scale. The yield of the product was 13.1 mg, and its estimated purity by LCMS analysis was 99.1%. Analysis condition A: Retention time=1.6 min; ESI-MS(+) m/z [M+H]⁺: 1888.

### Preparation of Compound 1281

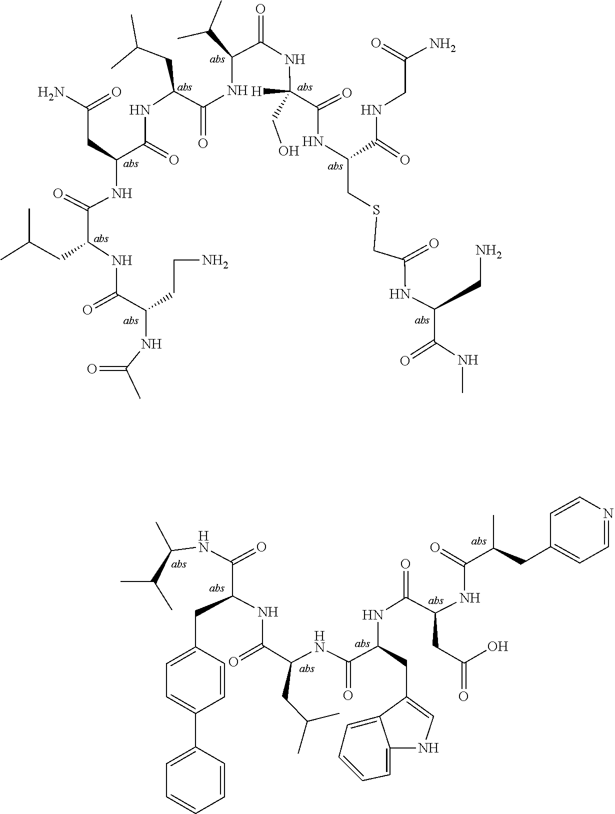 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1281 was prepared on a 50 μmol scale. The yield of the product was 18.1 mg, and its estimated purity by LCMS analysis was 99%. Analysis condition B: Retention time=1.38 min; ESI-MS(+) m/z [M+H]⁺: 1814.9.

### Preparation of Compound 1282

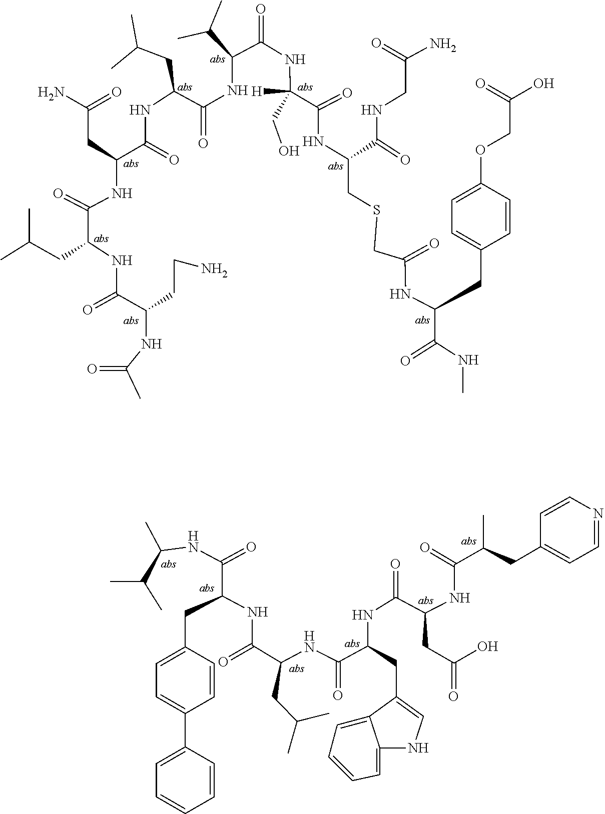 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1282 was prepared on a 50 μmol scale. The yield of the product was 49.6 mg, and its estimated purity by LCMS analysis was 97.4%. Analysis condition A: Retention time=1.61 min; ESI-MS(+) m/z [M+H]⁺: 1949.9.

### Preparation of Compound 1283

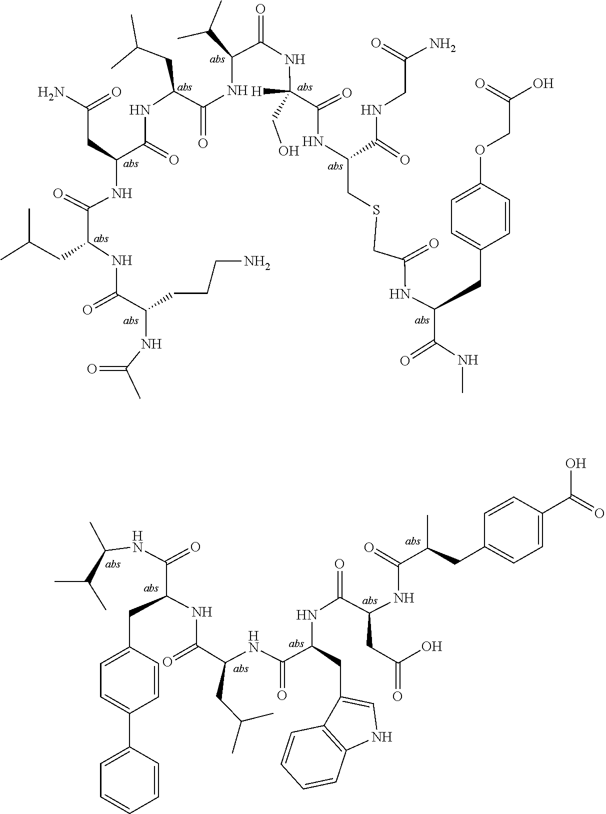 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(C(=O)O)cc1)C(=O)N[C@@H](Cc1ccc(-c2ccccc2)cc1)C(=O)N[C@H](C)C(C)C]

Compound 1283 was prepared on a 50 μmol scale. The yield of the product was 33.1 mg, and its estimated purity by LCMS analysis was 96.9%. Analysis condition A: Retention time=1.44, 1.55 min; ESI-MS(+) m/z [M+2H]²⁺: 1003.88, 1004.16.

### Preparation of Compound 1284

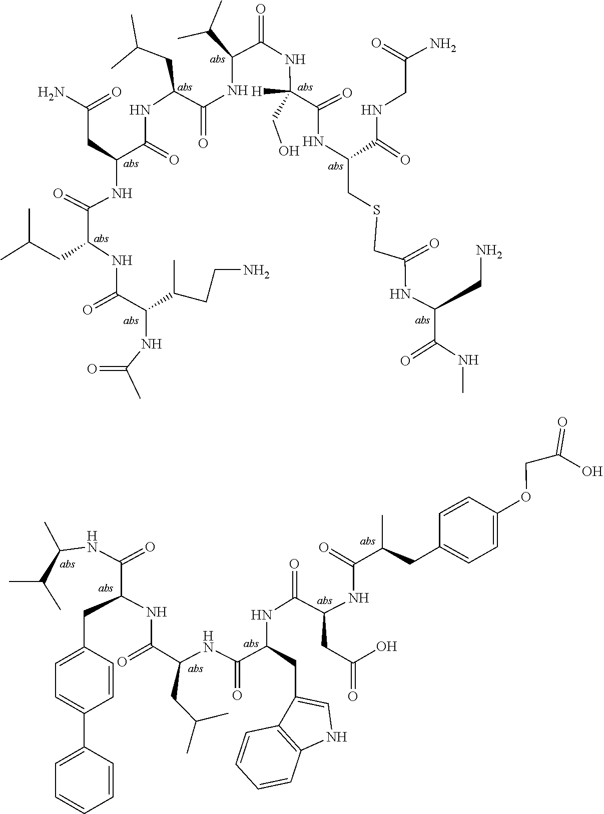 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@@H](NC(C)=O)C(C)CCN)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccc(OCC(=O)O)cc1)C(=O)N[C@@H](Cc1ccc(-c2ccccc2)cc1)C(=O)N[C@H](C)C(C)C]

Compound 1284 was prepared on a 50 μmol scale. The yield of the product was 4 mg, and its estimated purity by LCMS analysis was 93.3%. Analysis condition B: Retention time=1.55 min; ESI-MS(+) m/z [M+2H]²⁺: 952.

### Preparation of Compound 1285

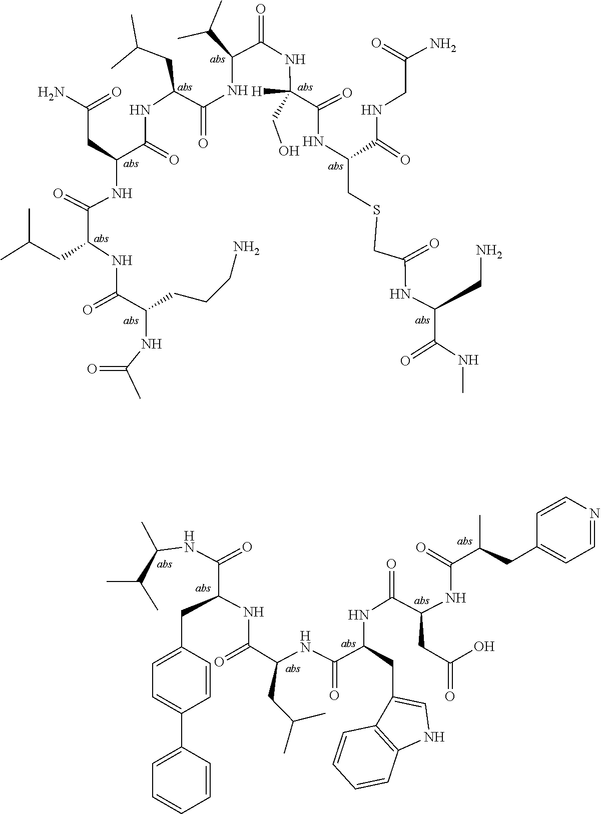 [SMILES: CNC(=O)[C@H](CN)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1285 was prepared on a 50 μmol scale. The yield of the product was 6.2 mg, and its estimated purity by LCMS analysis was 93.8%. Analysis condition B: Retention time=1.37 min; ESI-MS(+) m/z [M+3H]³⁺: 610.3.

### Preparation of Compound 1286

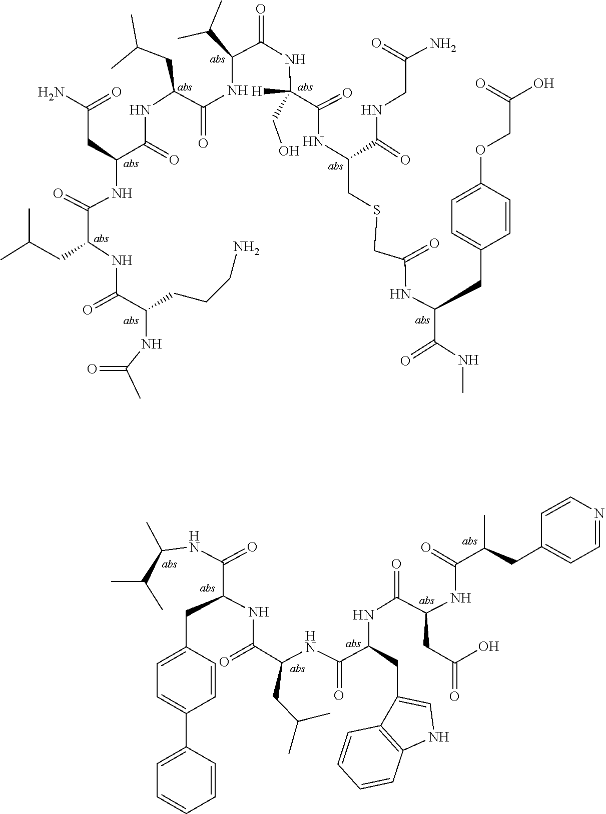 [SMILES: CNC(=O)[C@H](Cc1ccc(OCC(=O)O)cc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CCCN)NC(C)=O)C(C)C)C(=O)NCC(N)=O ; CC(C)C[C@H](NC(=O)[C@H](Cc1c[nH]c2ccccc12)NC(=O)[C@H](CC(=O)O)NC(=O)[C@@H](C)Cc1ccncc1)C(=O)N[C@@H](Cc1ccc(-c2ccccc2)cc1)C(=O)N[C@H](C)C(C)C]

Compound 1286 was prepared on a 50 μmol scale. The yield of the product was 21.6 mg, and its estimated purity by LCMS analysis was 96.8%. Analysis condition B: Retention time=1.51 min; ESI-MS(+) m/z [M+2H]²⁺: 982.9.

### Preparation of Compound 1287

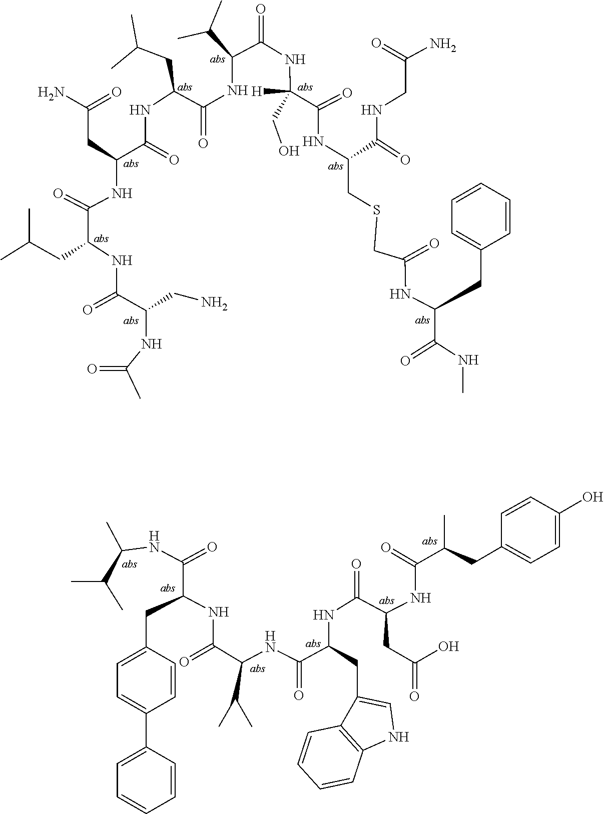 [SMILES: CNC(=O)[C@H](Cc1ccccc1)NC(=O)CSC[C@H](NC(=O)[C@H](CO)NC(=O)[C@@H](NC(=O)[C@H](CC(C)C)NC(=O)[C@H](CC(N)=O)NC(=O)[C@@H](CC(C)C)NC(=O)[C@H](CN)NC(C)=O)C(C)C)C(=O)NCC(N)=O ; CC(C)[C@H](NC(=O)[C@H](Cc1c[nH]c2ccccc12)NC(=O)[C@H](CC(=O)O)NC(=O)[C@@H](C)Cc1ccc(O)cc1)C(=O)N[C@@H](Cc1ccc(-c2ccccc2)cc1)C(=O)N[C@H](C)C(C)C]

Compound 1287 was prepared on a 50 μmol scale. The yield of the product was 16.5 mg, and its estimated purity by LCMS analysis was 96.4%. Analysis condition B: Retention time=1.7 min; ESI-MS(+) m/z [M+2H]²⁺: 933.1.

### Preparation of Compound 1288

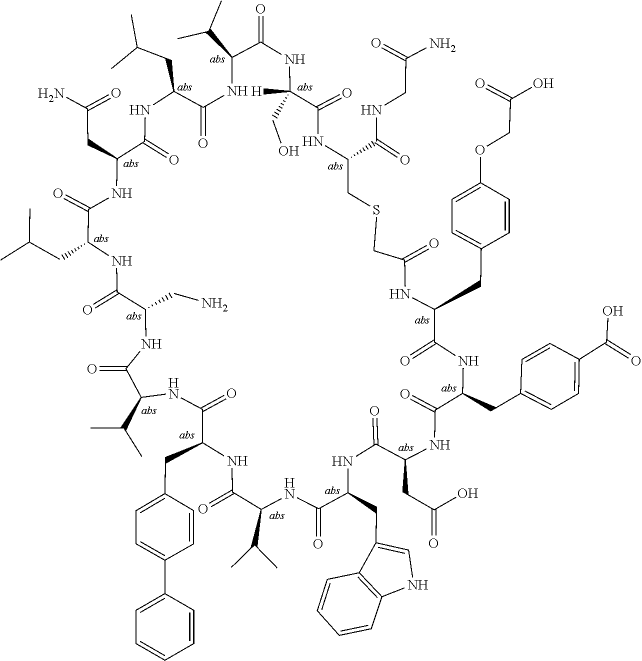 [SMILES: CC(C)C[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1288 was prepared on a 50 μmol scale. The yield of the product was 2.8 mg, and its estimated purity by LCMS analysis was 88.4%. Analysis condition B: Retention time=1.6, 1.63 min; ESI-MS(+) m/z [M+2H]²⁺: 983.09, 983.07.

### Preparation of Compound 1289

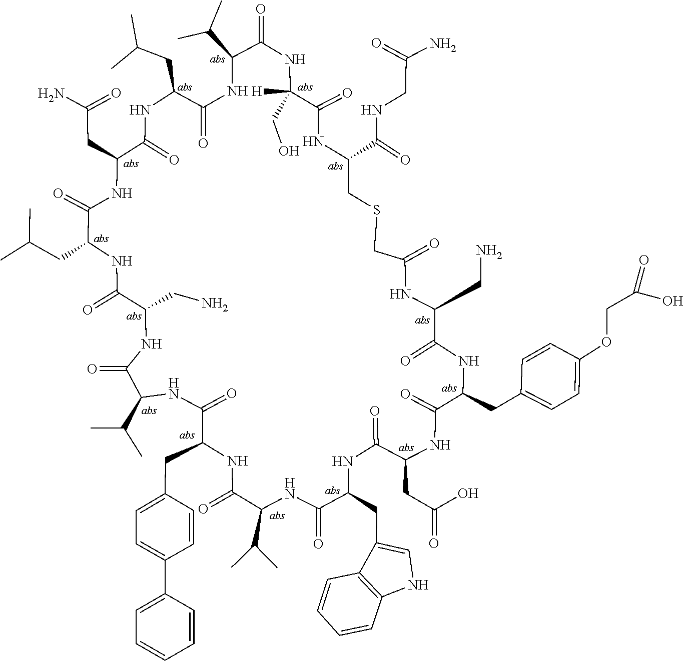 [SMILES: CC(C)C[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC1=O]

Compound 1289 was prepared on a 50 μmol scale. The yield of the product was 11.6 mg, and its estimated purity by LCMS analysis was 95.6%. Analysis condition B: Retention time=1.41 min; ESI-MS(+) m/z [M+2H]²⁺: 931.3.

### Preparation of Compound 1290

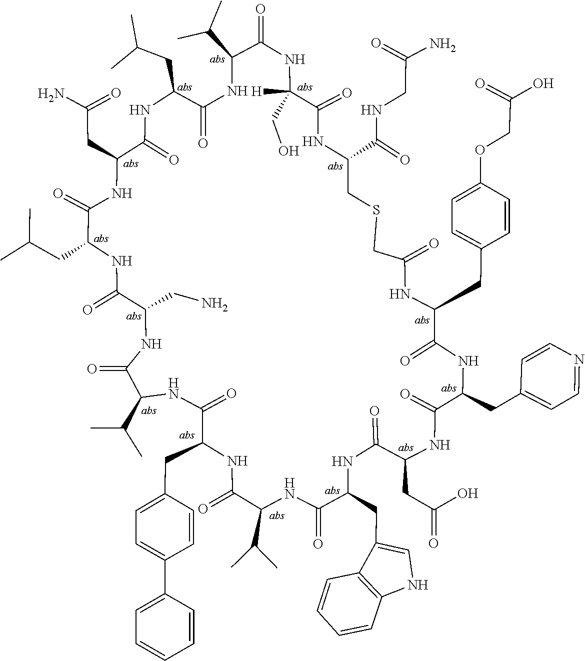 [SMILES: CC(C)C[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1290 was prepared on a 50 μmol scale. The yield of the product was 6.1 mg, and its estimated purity by LCMS analysis was 96.2%. Analysis condition B: Retention time=1.43 min; ESI-MS(+) m/z [M+2H]²⁺: 962.1.

### Preparation of Compound 1291

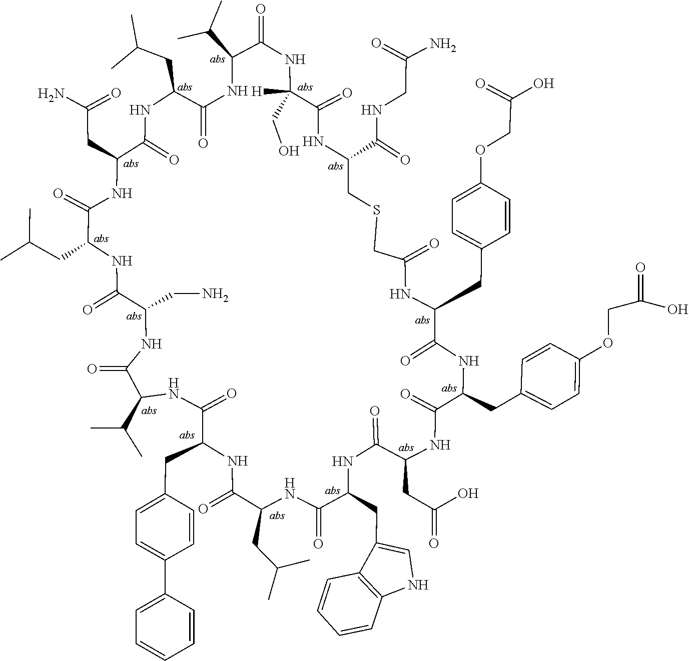 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC(C)C)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1291 was prepared on a 50 μmol scale. The yield of the product was 12.4 mg, and its estimated purity by LCMS analysis was 97.1%. Analysis condition B: Retention time=1.72 min; ESI-MS(+) m/z [M+2H]²⁺: 1005.1.

### Preparation of Compound 1292

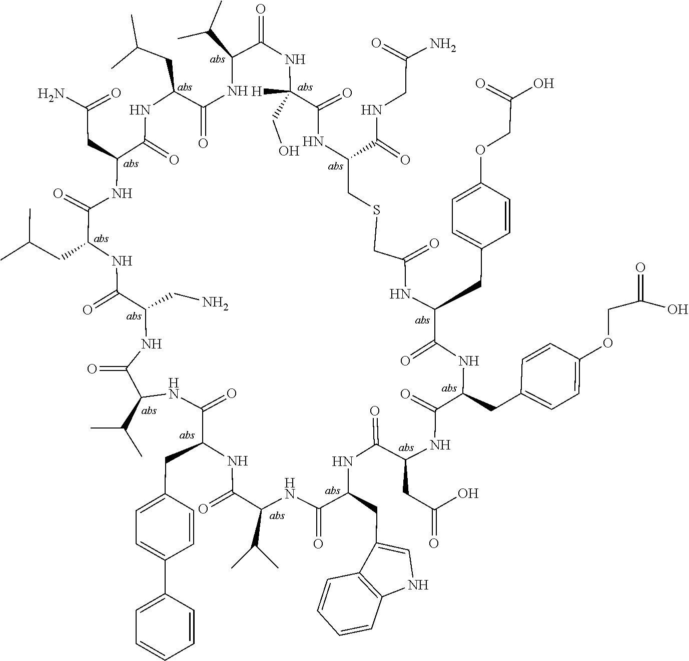 [SMILES: CC(C)C[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1292 was prepared on a 50 μmol scale. The yield of the product was 24.9 mg, and its estimated purity by LCMS analysis was 92.3%. Analysis condition B: Retention time=1.61 min; ESI-MS(+) m/z [M+2H]²⁺: 998.1.

### Preparation of Compound 1293

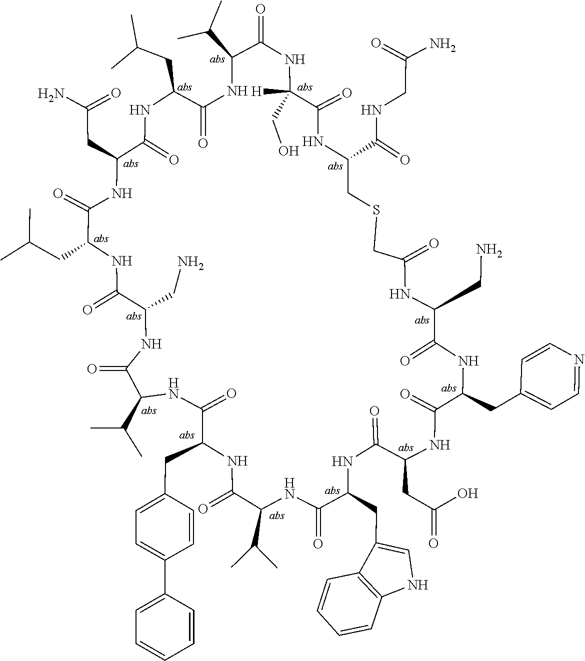 [SMILES: CC(C)C[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](CO)NC(=O)[C@H](C(C)C)NC1=O]

Compound 1293 was prepared on a 50 μmol scale. The yield of the product was 21.7 mg, and its estimated purity by LCMS analysis was 95.1%. Analysis condition A: Retention time=1.69 min; ESI-MS(+) m/z [M+2H]²⁺: 894.1.

### Preparation of Compound 1294

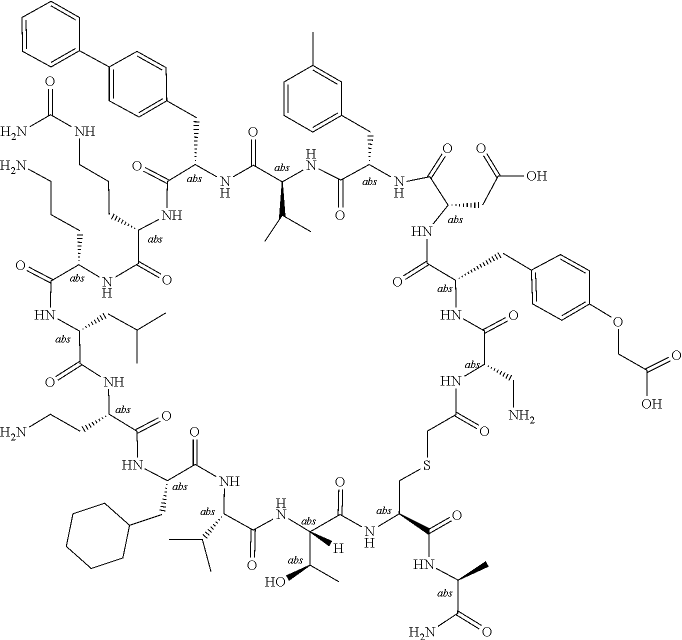 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1294 was prepared on a 50 μmol scale. The yield of the product was 4.8 mg, and its estimated purity by LCMS analysis was 95.8%. Analysis condition A: Retention time=1.51 min; ESI-MS(+) m/z [M+2H]²⁺: 995.7.

### Preparation of Compound 1295

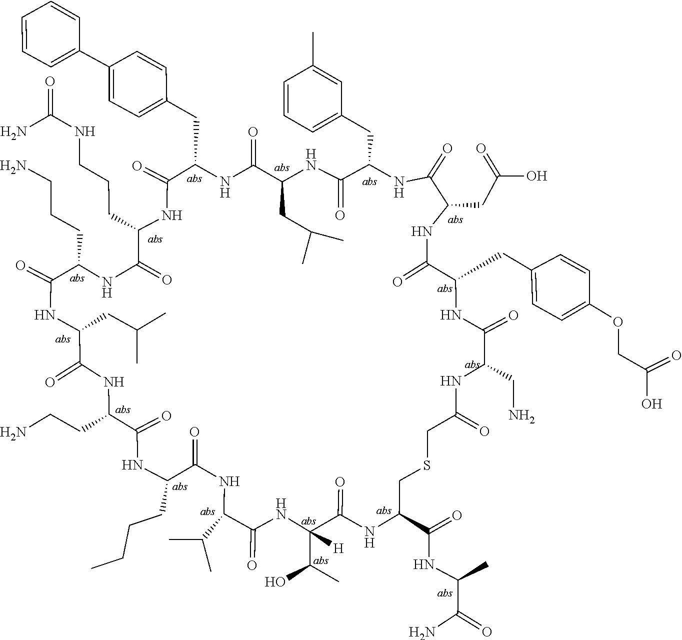 [SMILES: CCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1295 was prepared on a 50 μmol scale. The yield of the product was 12.9 mg, and its estimated purity by LCMS analysis was 93.8%. Analysis condition B: Retention time=1.31 min; ESI-MS(+) m/z [M+3H]³⁺: 650.8.

### Preparation of Compound 1296

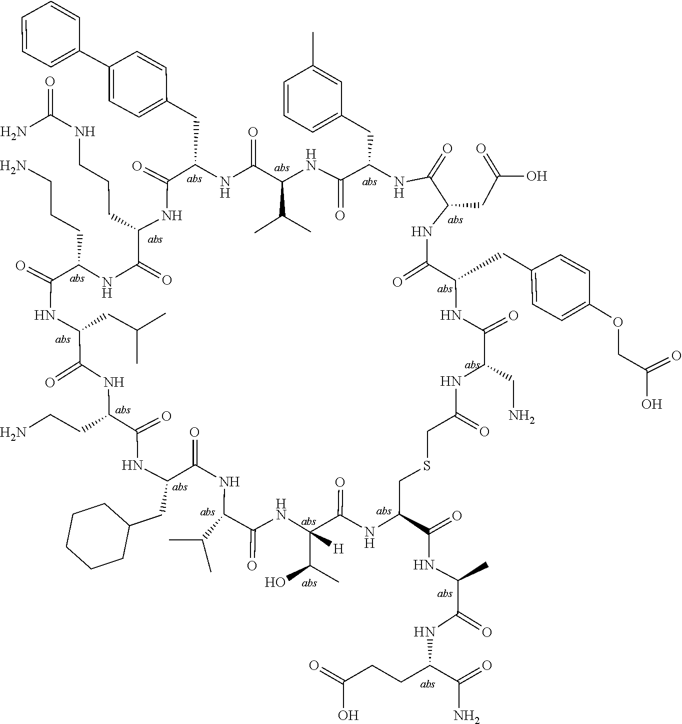 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1296 was prepared on a 50 μmol scale. The yield of the product was 20.7 mg, and its estimated purity by LCMS analysis was 94.7%. Analysis condition B: Retention time=1.48 min; ESI-MS(+) m/z [M+3H]³⁺: 702.2.

### Preparation of Compound 1297

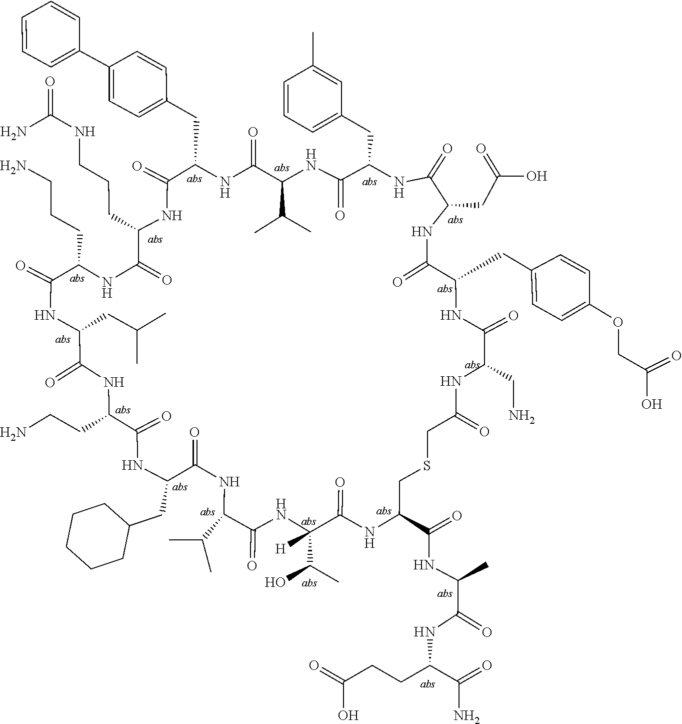 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)N[C@@H](CCC(=O)O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1297 was prepared on a 50 μmol scale. The yield of the product was 21.7 mg, and its estimated purity by LCMS analysis was 87.3%. Analysis condition B: Retention time=1.48 min; ESI-MS(+) m/z [M+2H]²⁺: 1053.4.

### Preparation of Compound 1298

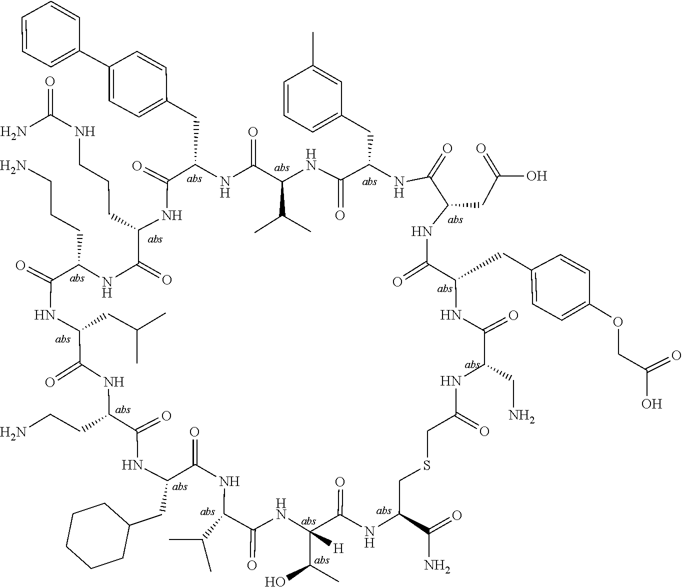 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1298 was prepared on a 50 μmol scale. The yield of the product was 15.7 mg, and its estimated purity by LCMS analysis was 93.5%. Analysis condition A: Retention time=1.45 min; ESI-MS(+) m/z [M+2H]²⁺: 953.

### Preparation of Compound 1299

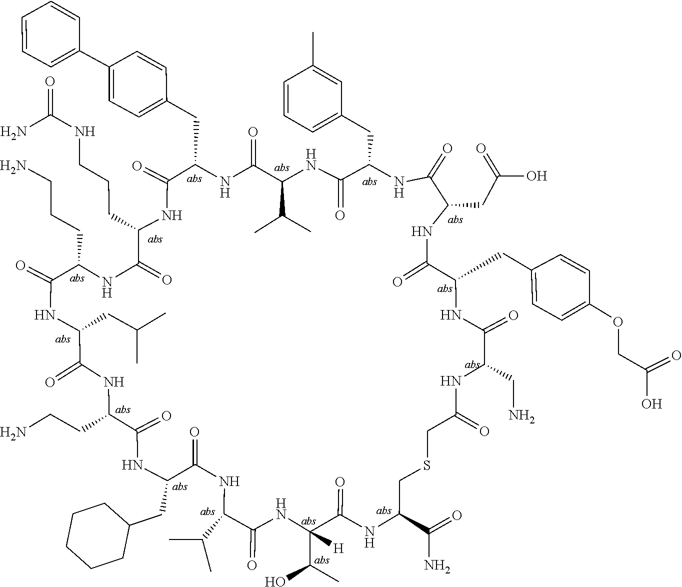 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1299 was prepared on a 50 μmol scale. The yield of the product was 18.6 mg, and its estimated purity by LCMS analysis was 95.8%. Analysis condition A: Retention time=1.46 min; ESI-MS(+) m/z [M+2H]²⁺: 953.7.

### Preparation of Compound 1300

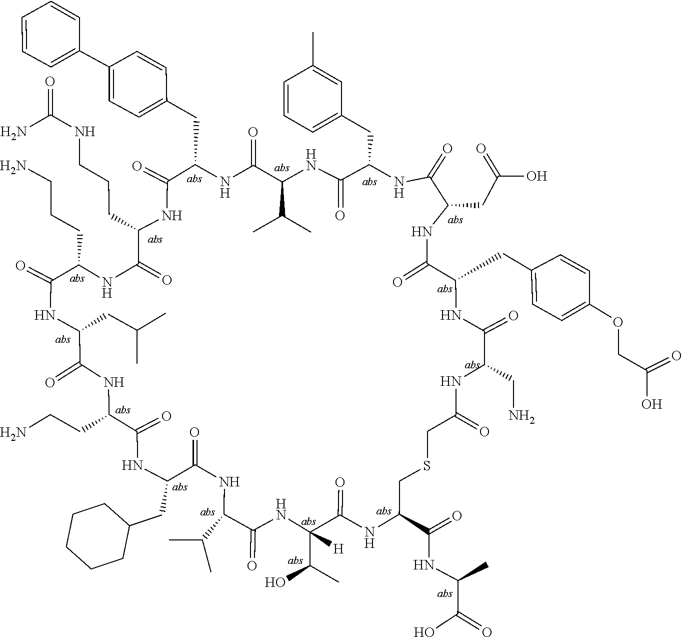 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1300 was prepared on a 50 μmol scale. The yield of the product was 26.9 mg, and its estimated purity by LCMS analysis was 83.7%. Analysis condition B: Retention time=1.51 min; ESI-MS(+) m/z [M+3H]³⁺: 659.8.

### Preparation of Compound 1301

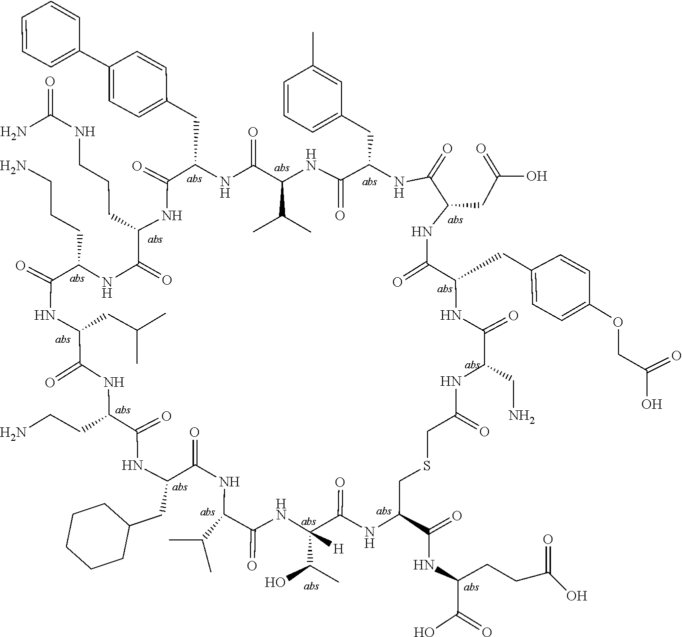 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCC(=O)O)C(=O)O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1301 was prepared on a 50 μmol scale. The yield of the product was 16.9 mg, and its estimated purity by LCMS analysis was 84.6%. Analysis condition B: Retention time=1.5 min; ESI-MS(+) m/z [M+3H]³⁺: 679.1.

### Preparation of Compound 1302

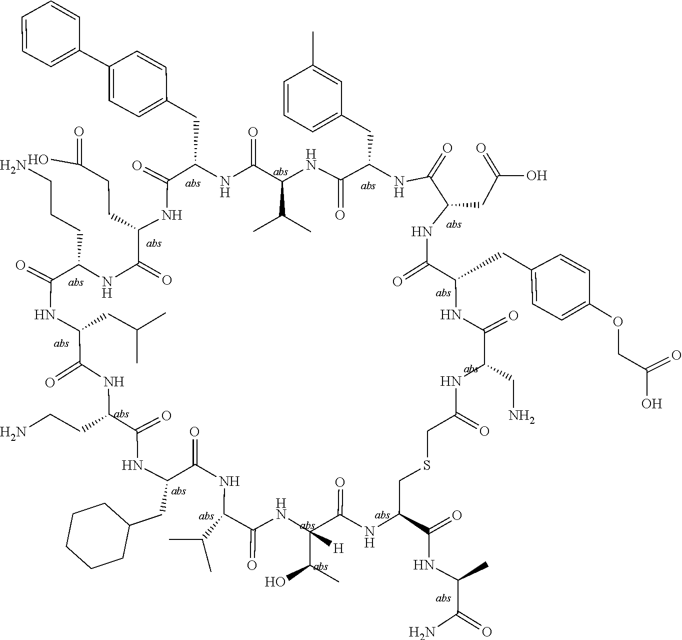 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1302 was prepared on a 50 μmol scale. The yield of the product was 29.7 mg, and its estimated purity by LCMS analysis was 88.6%. Analysis condition A: Retention time=1.45 min; ESI-MS(+) m/z [M+3H]³⁺: 650.2.

### Preparation of Compound 1303

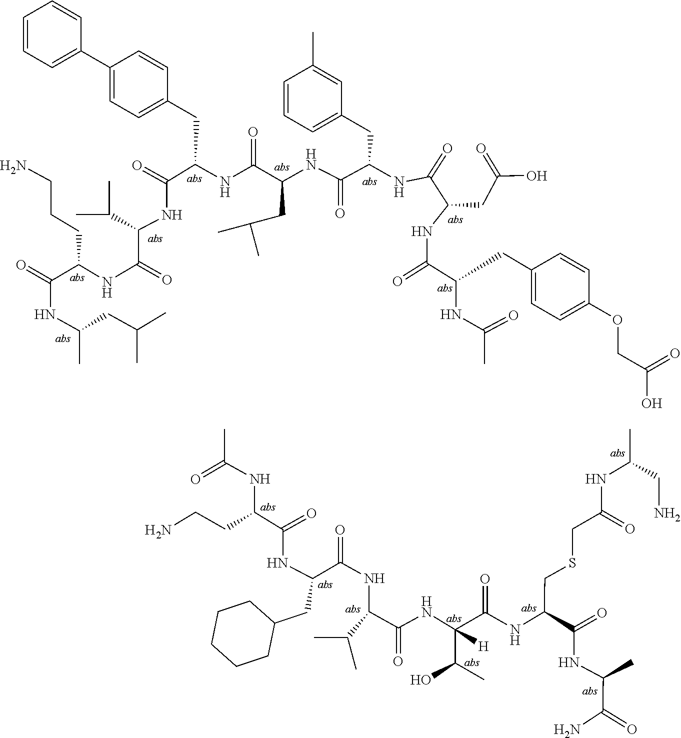

Compound 1303 was prepared on a 50 μmol scale. The yield of the product was 3 mg, and its estimated purity by LCMS analysis was 90.1%. Analysis condition B: Retention time=1.55 min; ESI-MS(+) m/z [M+2H]²⁺: 967.1.

### Preparation of Compound 1304

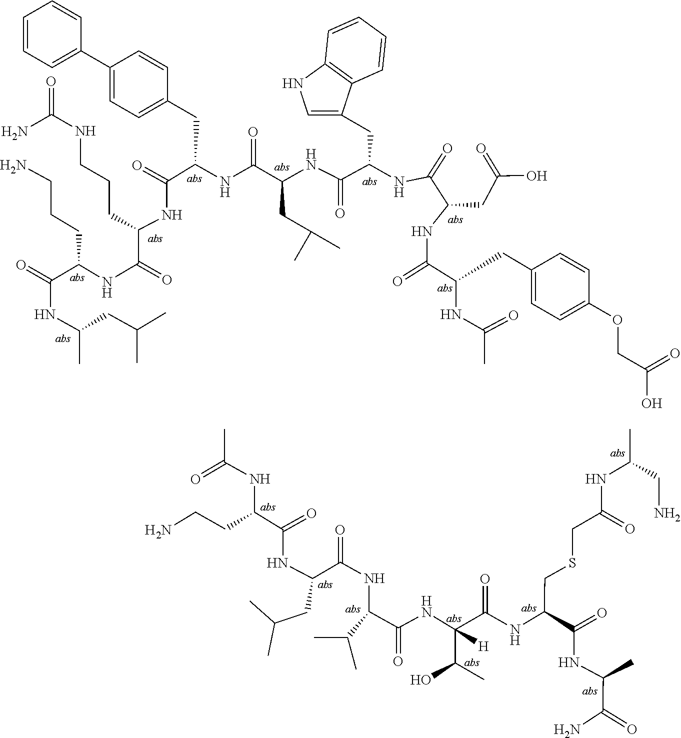

Compound 1304 was prepared on a 50 μmol scale. The yield of the product was 11.7 mg, and its estimated purity by LCMS analysis was 97.6%. Analysis condition B: Retention time=1.39 min; ESI-MS(+) m/z [M+3H]³⁺: 658.9.

### Preparation of Compound 1305

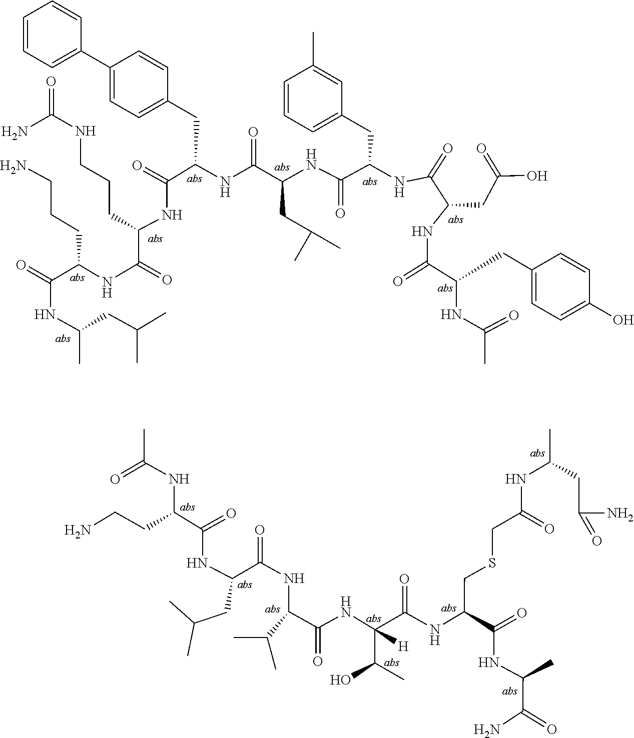 [SMILES: CC(=O)N[C@@H](Cc1ccc(O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C ; CC(=O)N[C@@H](CCN)C(=O)N[C@@H](CC(C)C)C(=O)N[C@H](C(=O)N[C@H](C(=O)N[C@@H](CSCC(=O)N[C@H](C)CC(N)=O)C(=O)N[C@@H](C)C(N)=O)[C@@H](C)O)C(C)C]

Compound 1305 was prepared on a 50 μmol scale. The yield of the product was 13.5 mg, and its estimated purity by LCMS analysis was 93.7%. Analysis condition B: Retention time=1.47 min; ESI-MS(+) m/z [M+2H]²⁺: 959.9.

### Preparation of Compound 1306

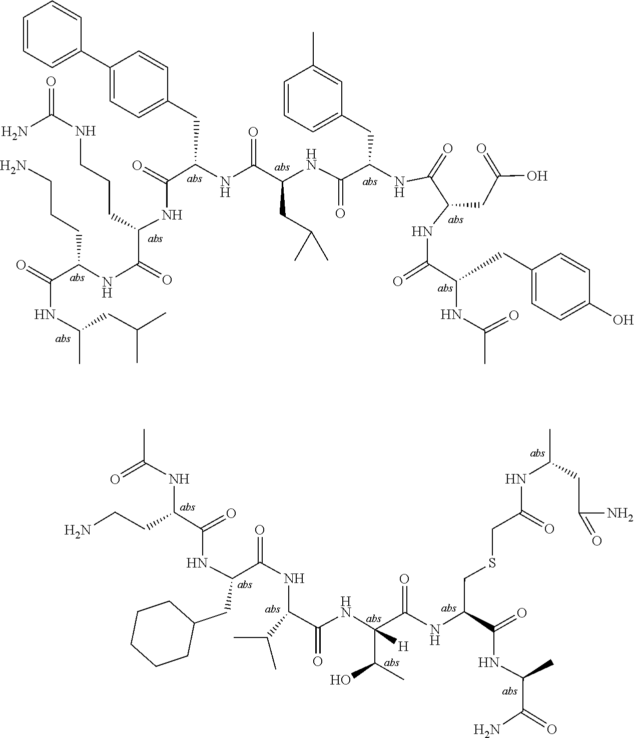 [SMILES: CC(=O)N[C@@H](Cc1ccc(O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C ; CC(=O)N[C@@H](CCN)C(=O)N[C@@H](CC1CCCCC1)C(=O)N[C@H](C(=O)N[C@H](C(=O)N[C@@H](CSCC(=O)N[C@H](C)CC(N)=O)C(=O)N[C@@H](C)C(N)=O)[C@@H](C)O)C(C)C]

Compound 1306 was prepared on a 50 μmol scale. The yield of the product was 4.2 mg, and its estimated purity by LCMS analysis was 91.8%. Analysis condition B: Retention time=1.64 min; ESI-MS(+) m/z [M+3H]³⁺: 653.8.

### Preparation of Compound 1307

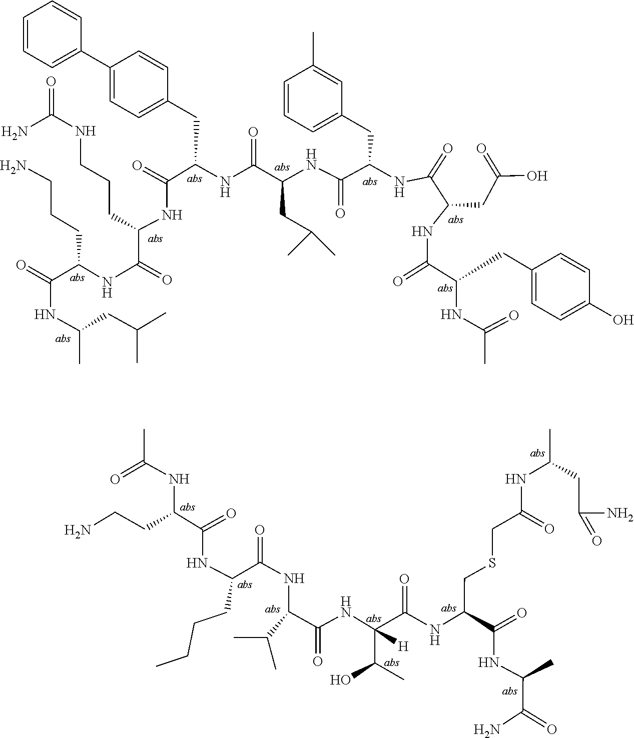 [SMILES: CC(=O)N[C@@H](Cc1ccc(O)cc1)C(=O)N[C@@H](CC(=O)O)C(=O)N[C@@H](Cc1cccc(C)c1)C(=O)N[C@@H](CC(C)C)C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C ; CCCC[C@H](NC(=O)[C@H](CCN)NC(C)=O)C(=O)N[C@H](C(=O)N[C@H](C(=O)N[C@@H](CSCC(=O)N[C@H](C)CC(N)=O)C(=O)N[C@@H](C)C(N)=O)[C@@H](C)O)C(C)C]

Compound 1307 was prepared on a 50 μmol scale. The yield of the product was 7.7 mg, and its estimated purity by LCMS analysis was 95.4%. Analysis condition A: Retention time=1.51 min; ESI-MS(+) m/z [M+2H]²⁺: 960.8.

### Preparation of Compound 1308

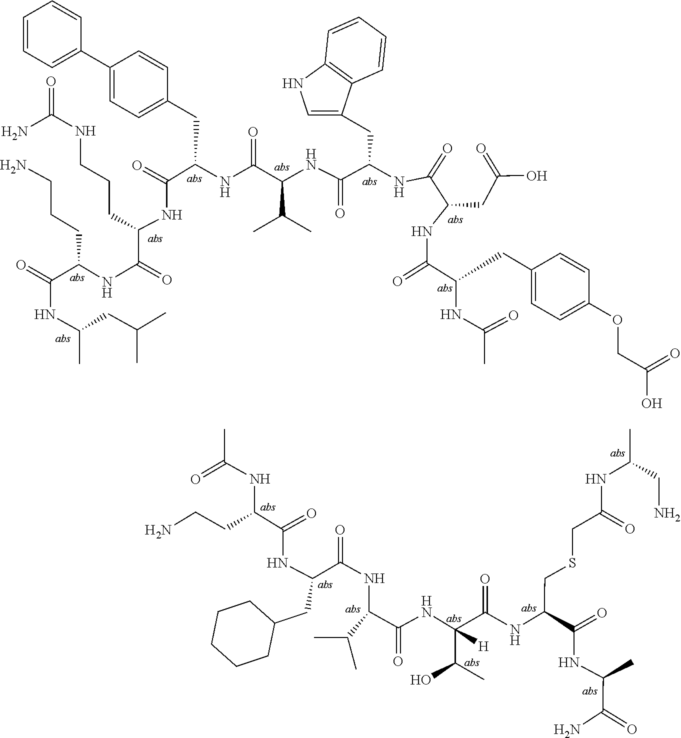

Compound 1308 was prepared on a 50 μmol scale. The yield of the product was 4.8 mg, and its estimated purity by LCMS analysis was 84.1%. Analysis condition B: Retention time=1.35 min; ESI-MS(+) m/z [M+2H]²⁺: 1001.1.

### Preparation of Compound 1309

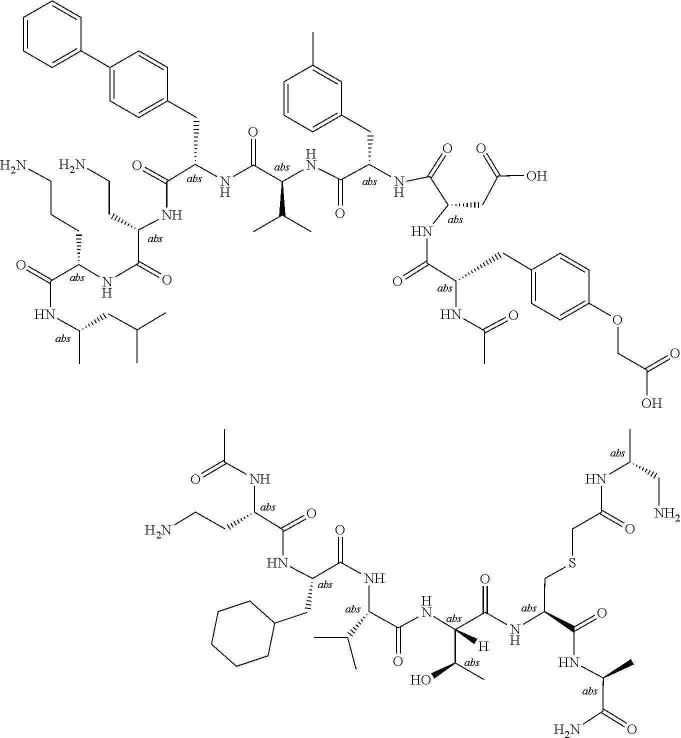

Compound 1309 was prepared on a 50 μmol scale. The yield of the product was 14.8 mg, and its estimated purity by LCMS analysis was 94.3%. Analysis condition B: Retention time=1.4 min; ESI-MS(+) m/z [M+3H]³⁺: 640.6.

### Preparation of Compound 1310

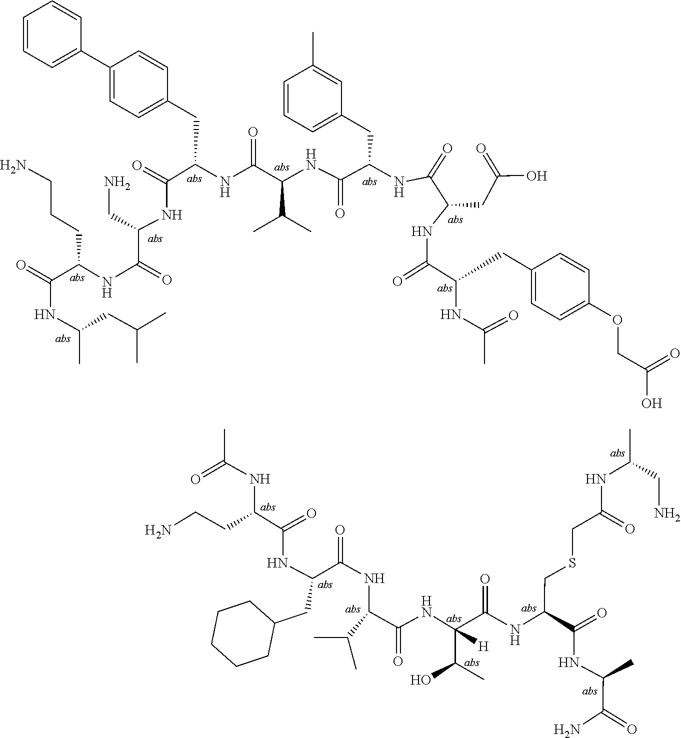

Compound 1310 was prepared on a 50 μmol scale. The yield of the product was 18.6 mg, and its estimated purity by LCMS analysis was 92.8%. Analysis condition A: Retention time=2.15 min; ESI-MS(+) m/z [M+2H]²⁺: 953.1.

### Preparation of Compound 1311

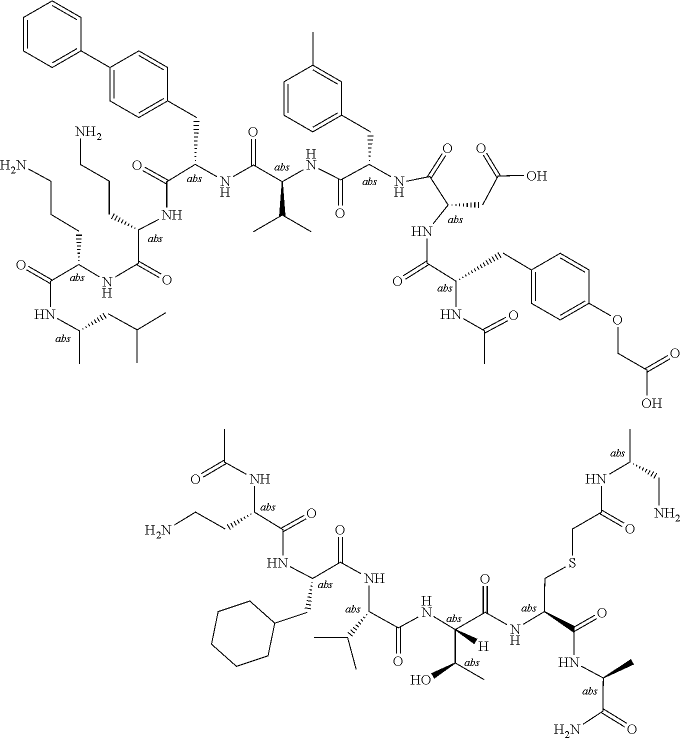

Compound 1311 was prepared on a 50 μmol scale. The yield of the product was 7.8 mg, and its estimated purity by LCMS analysis was 91.3%. Analysis condition B: Retention time=1.4 min; ESI-MS(+) m/z [M+2H]²⁺: 967.2.

### Preparation of Compound 1312

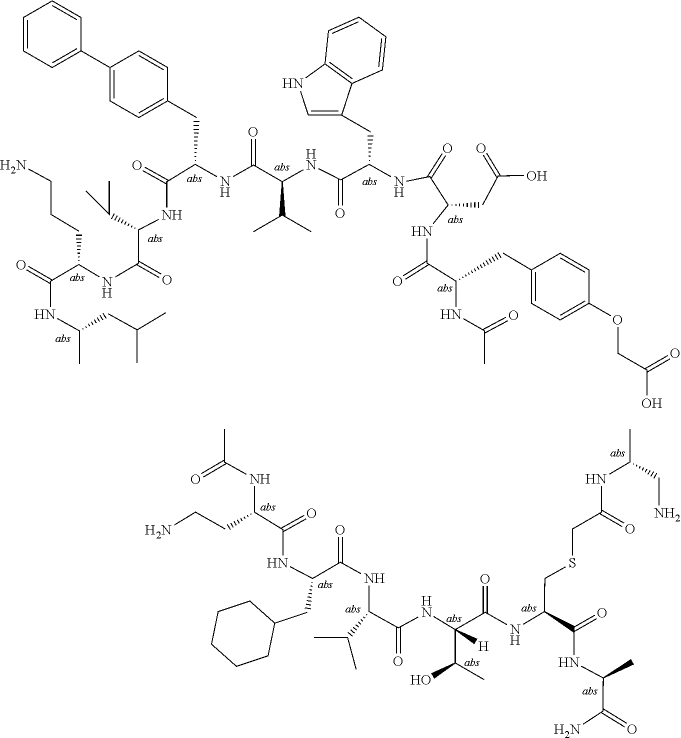

Compound 1312 was prepared on a 50 μmol scale. The yield of the product was 10.1 mg, and its estimated purity by LCMS analysis was 93.1%. Analysis condition A: Retention time=1.47 min; ESI-MS(+) m/z [M+2H]²⁺: 972.1.

### Preparation of Compound 1313

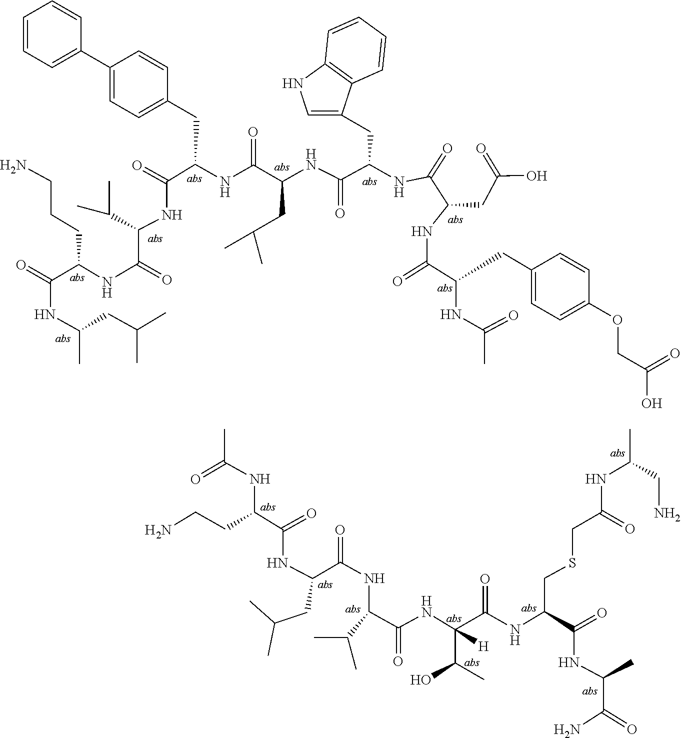

Compound 1313 was prepared on a 50 μmol scale. The yield of the product was 7.4 mg, and its estimated purity by LCMS analysis was 84.6%. Analysis condition A: Retention time=1.41 min; ESI-MS(+) m/z [M+2H]²⁺: 959.1.

### Preparation of Compound 1314

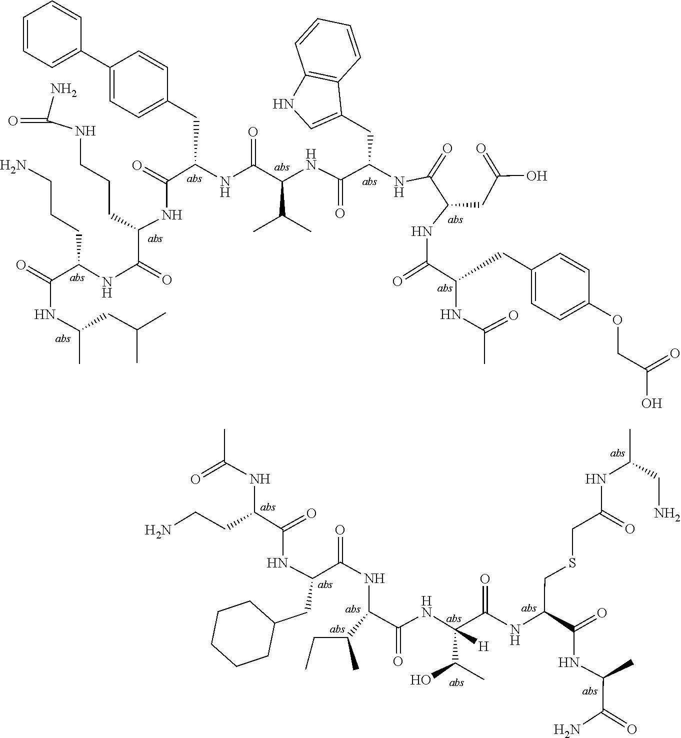

Compound 1314 was prepared on a 50 μmol scale. The yield of the product was 19.4 mg, and its estimated purity by LCMS analysis was 95.8%. Analysis condition B: Retention time=1.55 min; ESI-MS(+) m/z [M+2H]²⁺: 1008.

### Preparation of Compound 1315

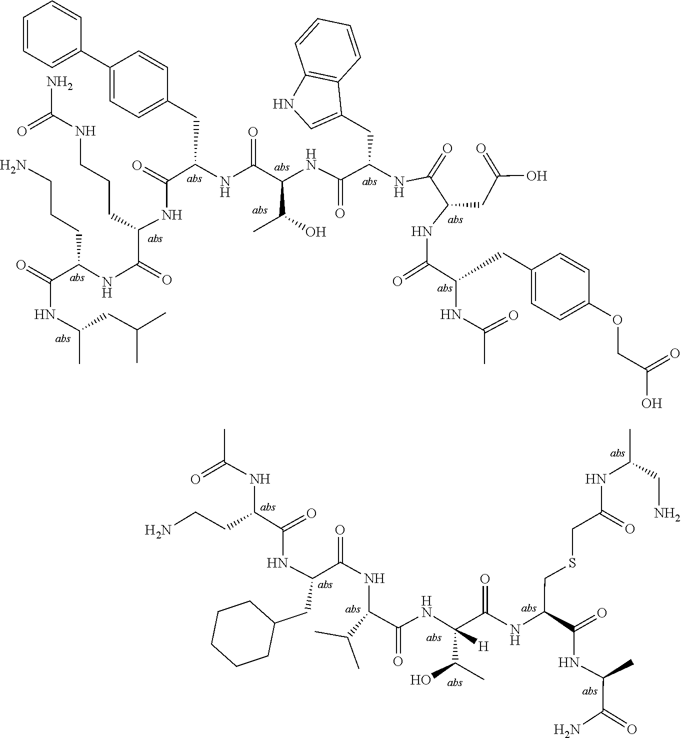

Compound 1315 was prepared on a 50 μmol scale. The yield of the product was 22.9 mg, and its estimated purity by LCMS analysis was 91%. Analysis condition B: Retention time=1.47 min; ESI-MS(+) m/z [M+2H]²⁺: 1001.1.

### Preparation of Compound 1316

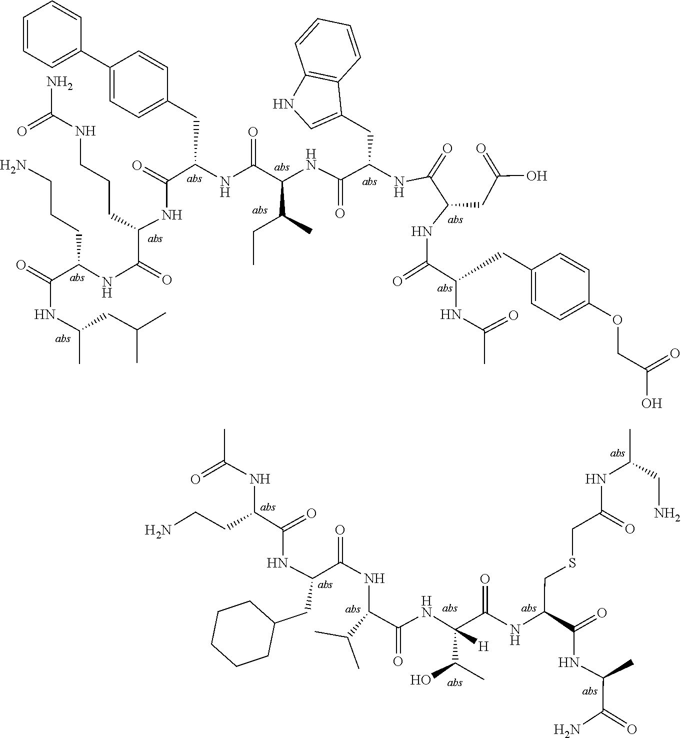

Compound 1316 was prepared on a 50 μmol scale. The yield of the product was 14 mg, and its estimated purity by LCMS analysis was 96.4%. Analysis condition B: Retention time=1.49 min; ESI-MS(+) m/z [M+2H]²⁺: 1007.9.

### Preparation of Compound 1317

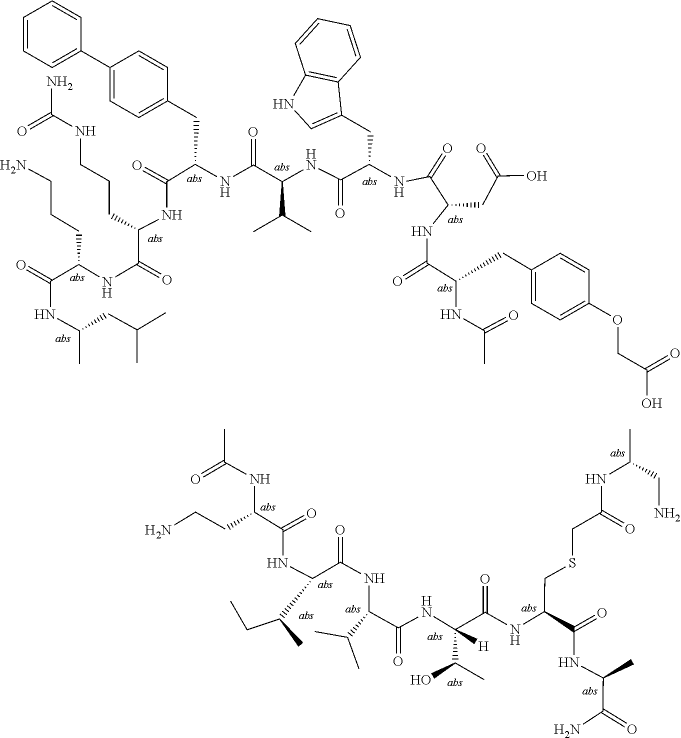

Compound 1317 was prepared on a 50 μmol scale. The yield of the product was 16.1 mg, and its estimated purity by LCMS analysis was 92.9%. Analysis condition B: Retention time=1.3 min; ESI-MS(+) m/z [M+3H]³⁺: 654.5.

### Preparation of Compound 1318

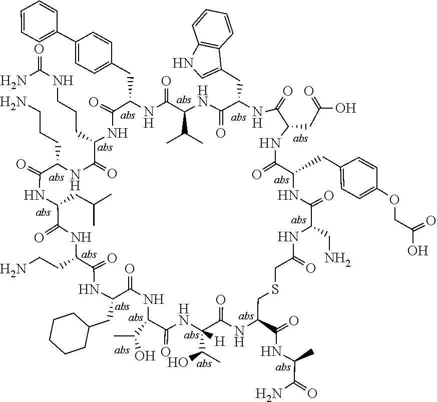 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H]([C@@H](C)O)NC(=O)[C@H](CC2CCCCC2)NC(=O)[C@H](CCN)NC1=O]

Compound 1318 was prepared on a 50 μmol scale. The yield of the product was 3.7 mg, and its estimated purity by LCMS analysis was 95.9%. Analysis condition B: Retention time=1.45 min; ESI-MS(+) m/z [M+3H]³⁺: 668.1.

### Preparation of Compound 1319

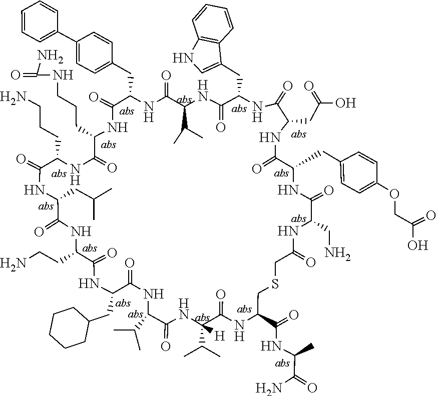 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](C(C)C)NC(=O)[C@H](C(C)C)NC(=O)[C@H](CC2CCCCC2)NC(=O)[C@H](CCN)NC1=O]

Compound 1319 was prepared on a 50 μmol scale. The yield of the product was 3.4 mg, and its estimated purity by LCMS analysis was 97.5%. Analysis condition A: Retention time=1.49 min; ESI-MS(+) m/z [M+2H]²⁺: 1000.1.

### Preparation of Compound 1320

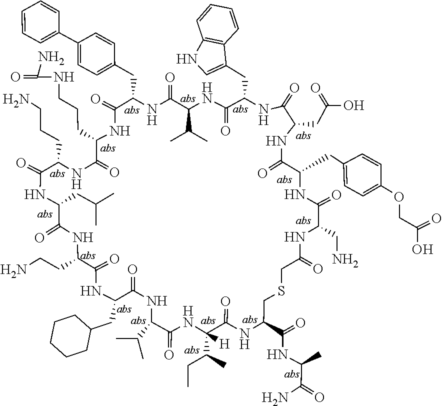 [SMILES: CC[C@H](C)[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC1=O]

Compound 1320 was prepared on a 50 μmol scale. The yield of the product was 12.1 mg, and its estimated purity by LCMS analysis was 97.6%. Analysis condition B: Retention time=1.55 min; ESI-MS(+) m/z [M+2H]²⁺: 1006.2.

### Preparation of Compound 1321

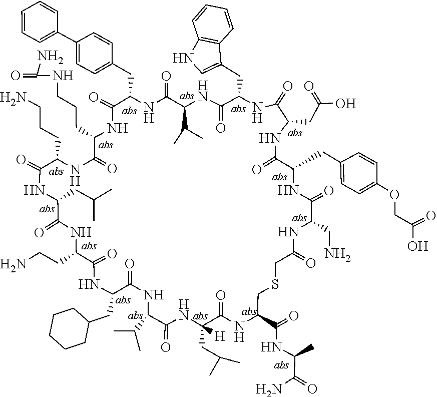 [SMILES: CC(C)C[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC1=O]

Compound 1321 was prepared on a 50 μmol scale. The yield of the product was 19.5 mg, and its estimated purity by LCMS analysis was 96.9%. Analysis condition B: Retention time=1.5 min; ESI-MS(+) m/z [M+2H]²⁺: 1007.2.

### Preparation of Compound 1322

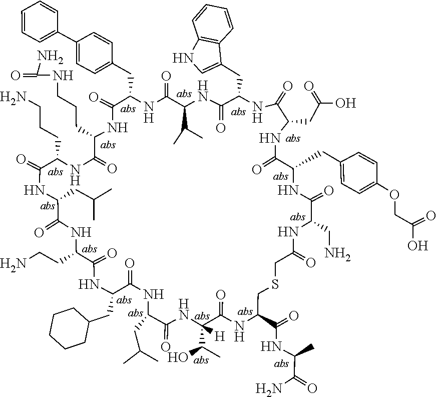 [SMILES: CC(C)C[C@@H]1NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC1=O]

Compound 1322 was prepared on a 50 μmol scale. The yield of the product was 2.9 mg, and its estimated purity by LCMS analysis was 86.4%. Analysis condition B: Retention time=1.58 min; ESI-MS(+) m/z [M+2H]²⁺: 1007.9.

### Preparation of Compound 1323

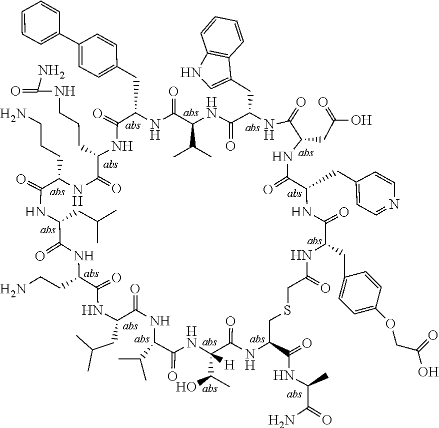 [SMILES: CC(C)C[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC1=O]

Compound 1323 was prepared on a 50 μmol scale. The yield of the product was 10.4 mg, and its estimated purity by LCMS analysis was 90.7%. Analysis condition B: Retention time=1.44 min; ESI-MS(+) m/z [M+2H]²⁺: 1012.2.

### Preparation of Compound 1324

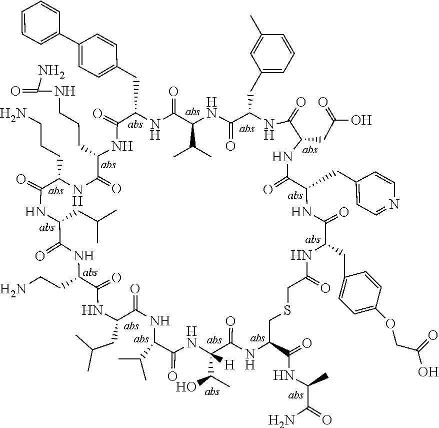 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1324 was prepared on a 50 μmol scale. The yield of the product was 12.8 mg, and its estimated purity by LCMS analysis was 89.8%. Analysis condition B: Retention time=1.33, 1.38 min; ESI-MS(+) m/z [M+H]⁺: 1997.

### Preparation of Compound 1325

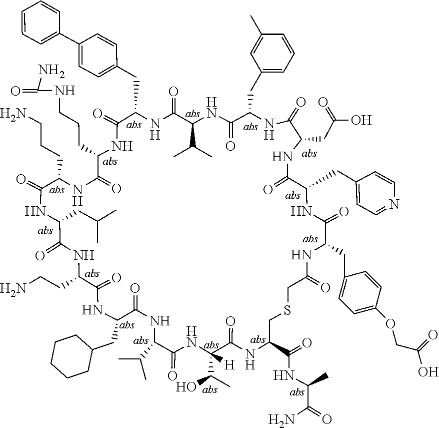 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1325 was prepared on a 50 μmol scale. The yield of the product was 7.8 mg, and its estimated purity by LCMS analysis was 93.8%. Analysis condition A: Retention time=1.69 min; ESI-MS(+) m/z [M+3H]³⁺: 680.2.

### Preparation of Compound 1326

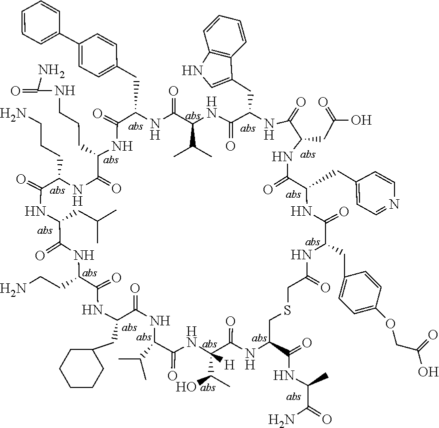 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1326 was prepared on a 50 μmol scale. The yield of the product was 14.2 mg, and its estimated purity by LCMS analysis was 92.2%. Analysis condition B: Retention time=1.54 min; ESI-MS(+) m/z [M+2H]²⁺: 1032.

### Preparation of Compound 1327

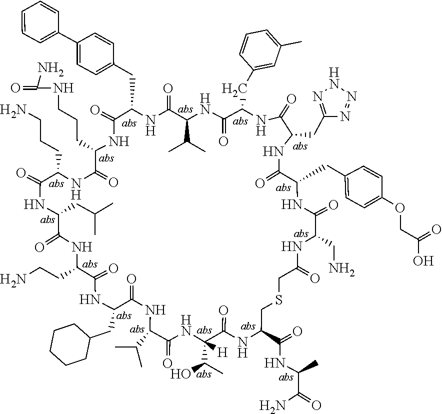 [SMILES: Cc1cccc(C[C@@H]2NC(=O)[C@H](Cc3nn[nH]n3)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1327 was prepared on a 50 μmol scale. The yield of the product was 5.3 mg, and its estimated purity by LCMS analysis was 97.8%. Analysis condition B: Retention time=1.6 min; ESI-MS(+) m/z [M+2H]²⁺: 1000.3.

### Preparation of Compound 1328

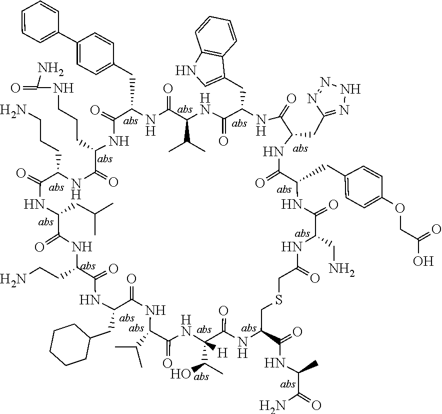 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](Cc2nn[nH]n2)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1328 was prepared on a 50 μmol scale. The yield of the product was 14.4 mg, and its estimated purity by LCMS analysis was 98.7%. Analysis condition A: Retention time=1.51 min; ESI-MS(+) m/z [M+2H]²⁺: 1013.

### Preparation of Compound 1329

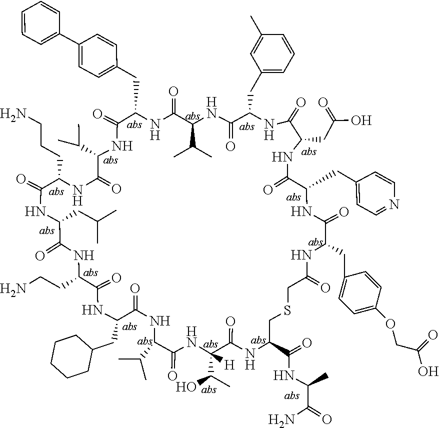 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1329 was prepared on a 50 μmol scale. The yield of the product was 24.7 mg, and its estimated purity by LCMS analysis was 93.4%. Analysis condition B: Retention time=1.5 min; ESI-MS(+) m/z [M+2H]²⁺: 990.4.

### Preparation of Compound 1330

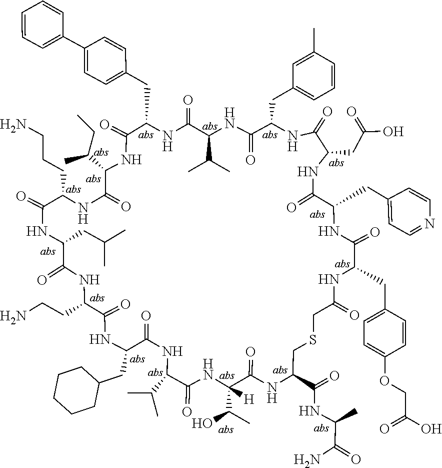 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1330 was prepared on a 50 μmol scale. The yield of the product was 5.7 mg, and its estimated purity by LCMS analysis was 95.9%. Analysis condition A: Retention time=2.16 min; ESI-MS(+) m/z [M+H]⁺: 1993.1.

### Preparation of Compound 1331

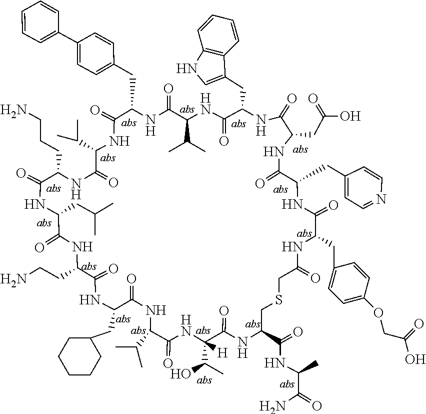 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1331 was prepared on a 50 μmol scale. The yield of the product was 35 mg, and its estimated purity by LCMS analysis was 90.9%. Analysis condition A: Retention time=1.96 min; ESI-MS(+) m/z [M+2H]²⁺: 1003.

### Preparation of Compound 1332

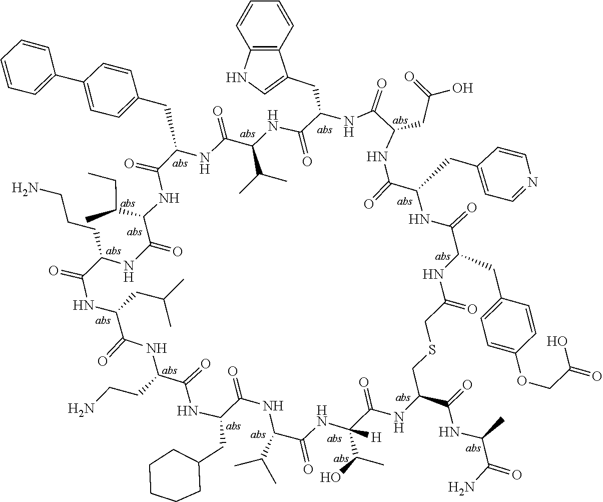 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1332 was prepared on a 50 μmol scale. The yield of the product was 3.2 mg, and its estimated purity by LCMS analysis was 95.5%. Analysis condition A: Retention time=1.98 min; ESI-MS(+) m/z [M+2H]²⁺: 1010.2.

### Preparation of Compound 1333

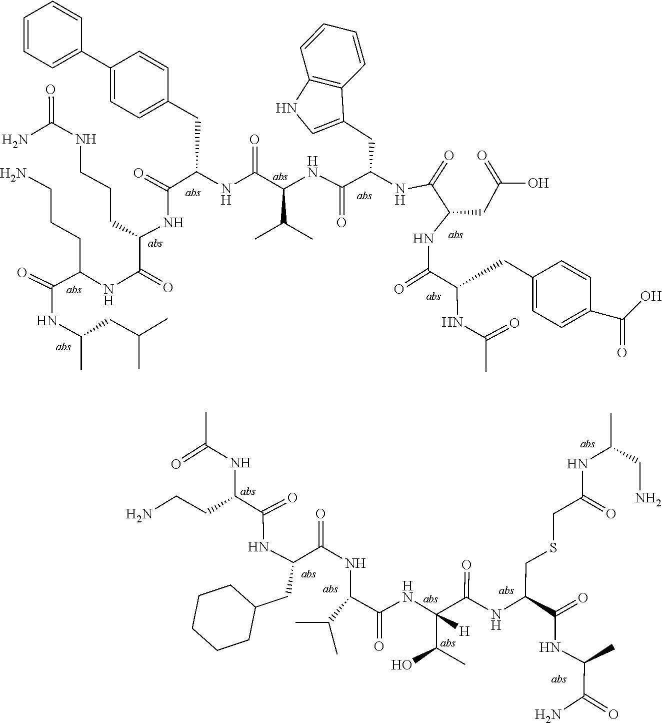 [SMILES: CC(=O)N[C@@H](Cc1ccc(C(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)NC(CCCN)C(=O)N[C@@H](C)CC(C)C)C(C)C ; CC(=O)N[C@@H](CCN)C(=O)N[C@@H](CC1CCCCC1)C(=O)N[C@H](C(=O)N[C@H](C(=O)N[C@@H](CSCC(=O)N[C@H](C)CN)C(=O)N[C@@H](C)C(N)=O)[C@@H](C)O)C(C)C]

Compound 1333 was prepared on a 50 μmol scale. The yield of the product was 15.8 mg, and its estimated purity by LCMS analysis was 94.2%. Analysis condition B: Retention time=1.47 min; ESI-MS(+) m/z [M+H]⁺: 1971.

### Preparation of Compound 1334

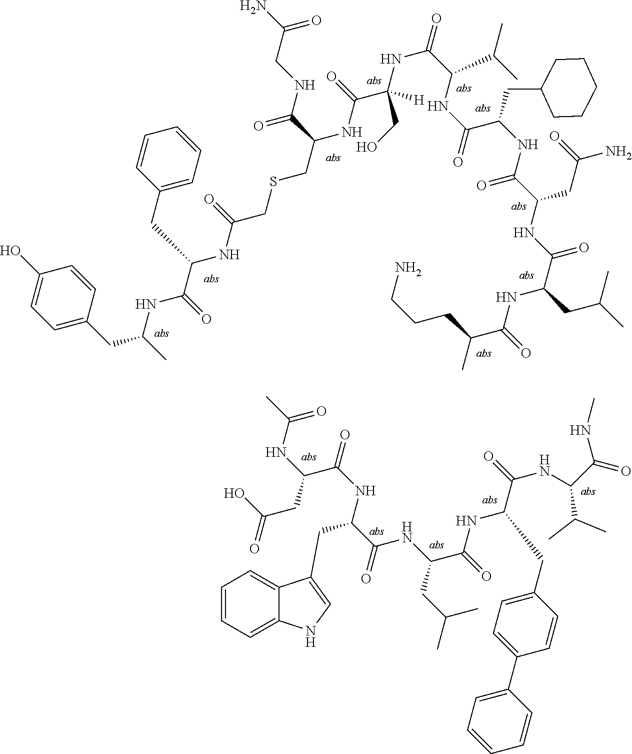

Compound 1334 was prepared on a 50 μmol scale. The yield of the product was 10.2 mg, and its estimated purity by LCMS analysis was 99.2%. Analysis condition A: Retention time=1.9 min; ESI-MS(+) m/z [M+2H]²⁺: 973.1.

### Preparation of Compound 1335

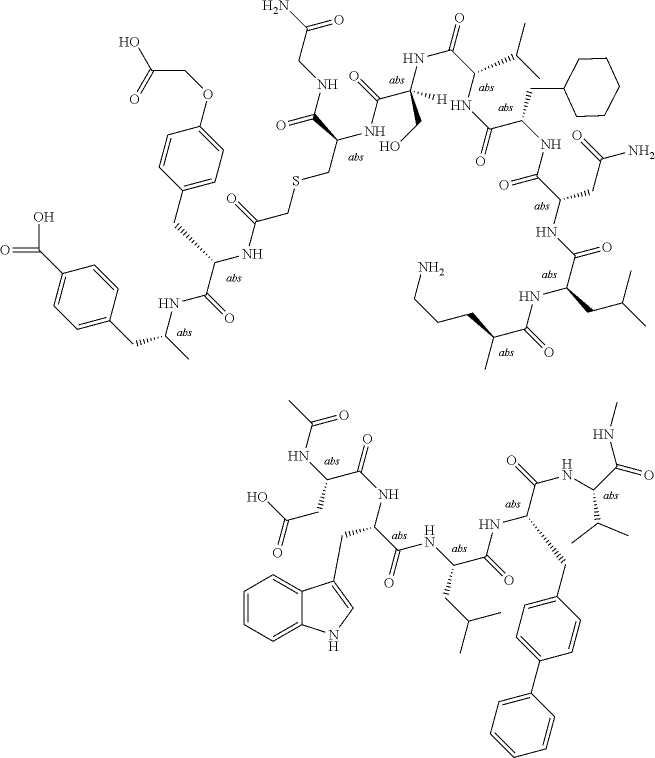

Compound 1335 was prepared on a 50 μmol scale. The yield of the product was 17.4 mg, and its estimated purity by LCMS analysis was 96.9%. Analysis condition B: Retention time=1.74 min; ESI-MS(+) m/z [M+2H]²⁺: 1024.8.

### Preparation of Compound 1336

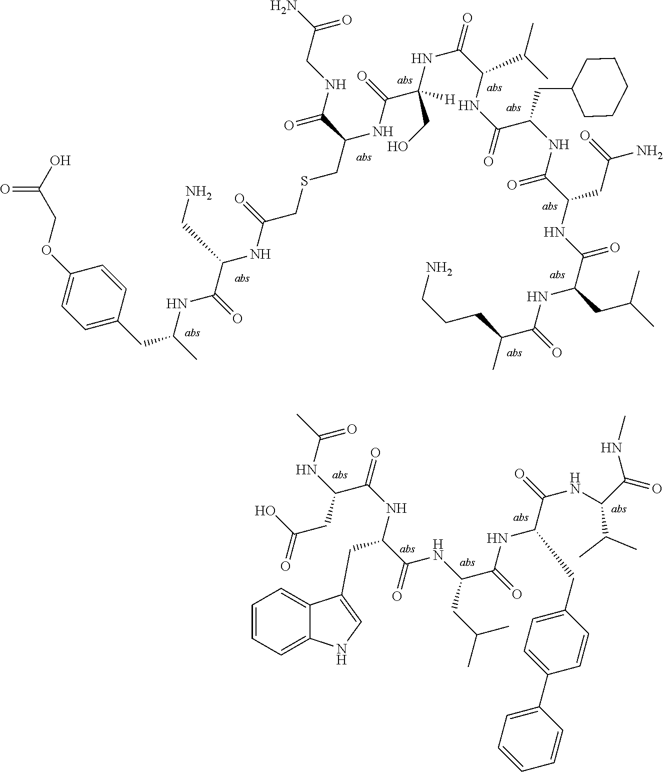

Compound 1336 was prepared on a 50 μmol scale. The yield of the product was 26.3 mg, and its estimated purity by LCMS analysis was 99.2%. Analysis condition B: Retention time=1.73 min; ESI-MS(+) m/z [M+H]⁺: 1941.6.

### Preparation of Compound 1337

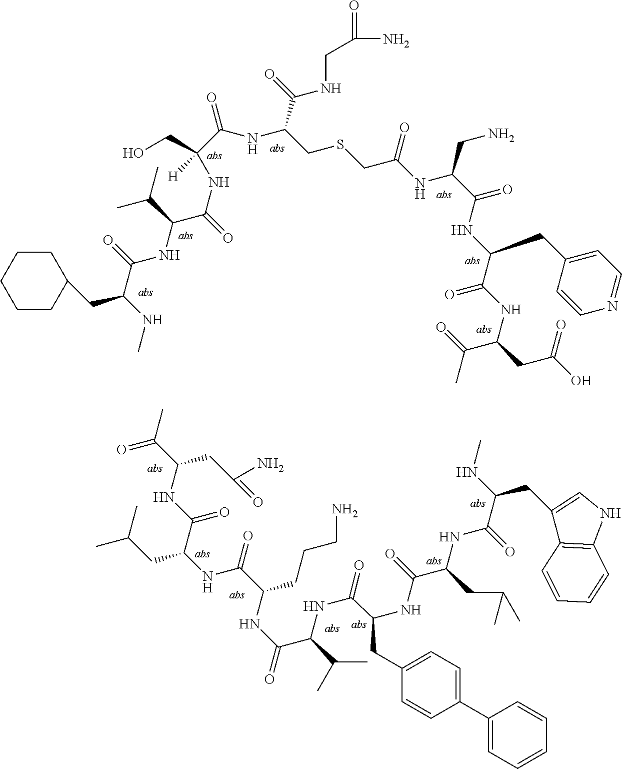 [SMILES: CN[C@@H](CC1CCCCC1)C(=O)N[C@H](C(=O)N[C@@H](CO)C(=O)N[C@@H](CSCC(=O)N[C@@H](CN)C(=O)N[C@@H](Cc1ccncc1)C(=O)N[C@@H](CC(=O)O)C(C)=O)C(=O)NCC(N)=O)C(C)C ; CN[C@@H](Cc1c[nH]c2ccccc12)C(=O)N[C@@H](CC(C)C)C(=O)N[C@@H](Cc1ccc(-c2ccccc2)cc1)C(=O)N[C@H](C(=O)N[C@@H](CCCN)C(=O)N[C@H](CC(C)C)C(=O)N[C@@H](CC(N)=O)C(C)=O)C(C)C]

Compound 1337 was prepared on a 50 μmol scale. The yield of the product was 20 mg, and its estimated purity by LCMS analysis was 86.6%. Analysis condition B: Retention time=1.56 min; ESI-MS(+) m/z [M+H]⁺: 1869.

### Preparation of Compound 1338

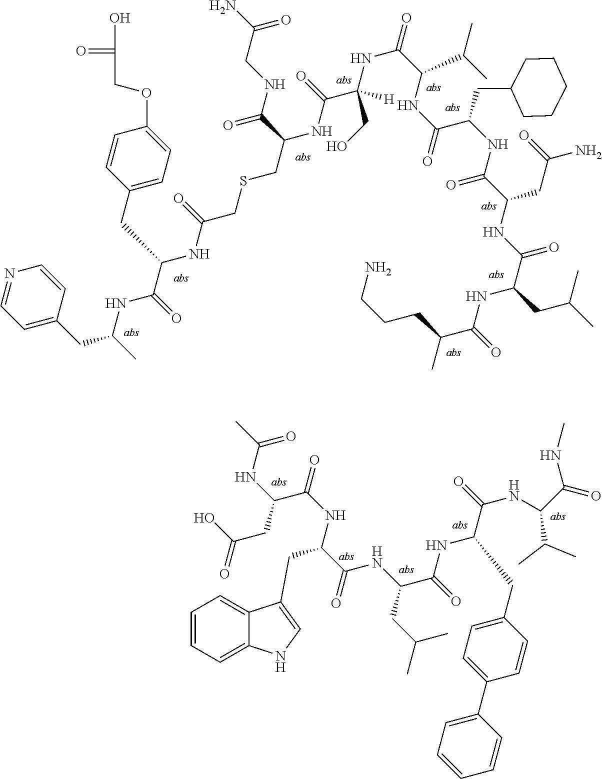

Compound 1338 was prepared on a 50 μmol scale. The yield of the product was 20.6 mg, and its estimated purity by LCMS analysis was 95.8%. Analysis condition A: Retention time=1.62 min; ESI-MS(+) m/z [M+2H]²⁺: 1003.

### Preparation of Compound 1339

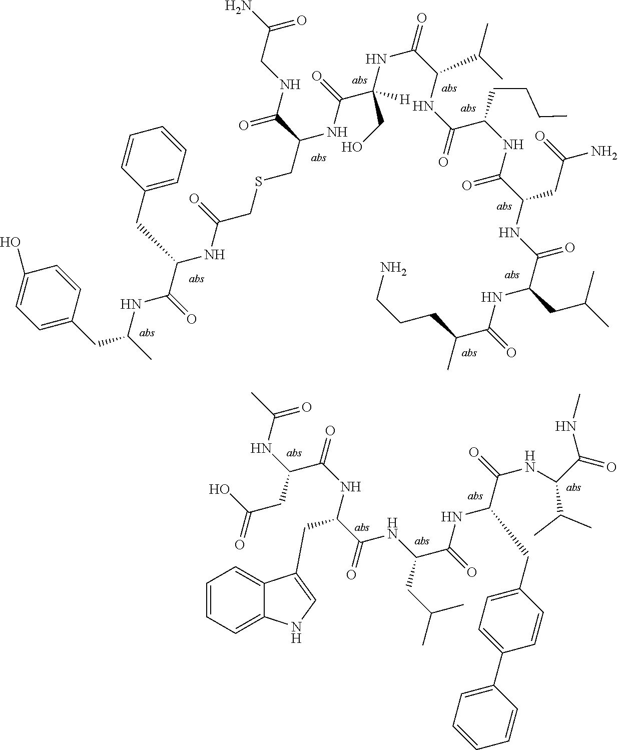

Compound 1339 was prepared on a 50 μmol scale. The yield of the product was 9.6 mg and its estimated purity by LCMS analysis was 97.3%. Analysis condition B: Retention time=1.77 min; ESI-MS(+) m/z [M+2H]²⁺: 953.5.

### Preparation of Compound 1340

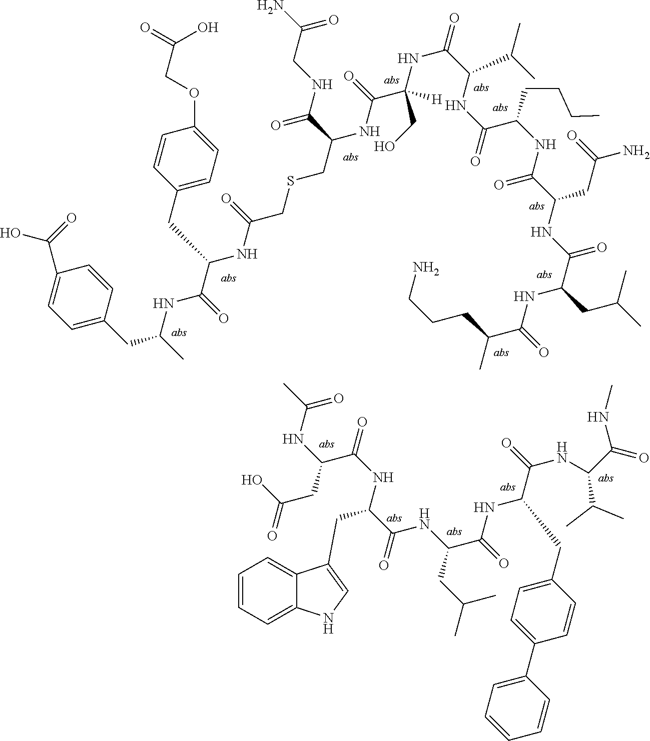

Compound 1340 was prepared on a 50 μmol scale. The yield of the product was 2.9 mg, and its estimated purity by LCMS analysis was 93%. Analysis condition B: Retention time=2.47 min; ESI-MS(+) m/z [M+2H]²⁺: 1004.

### Preparation of Compound 1341

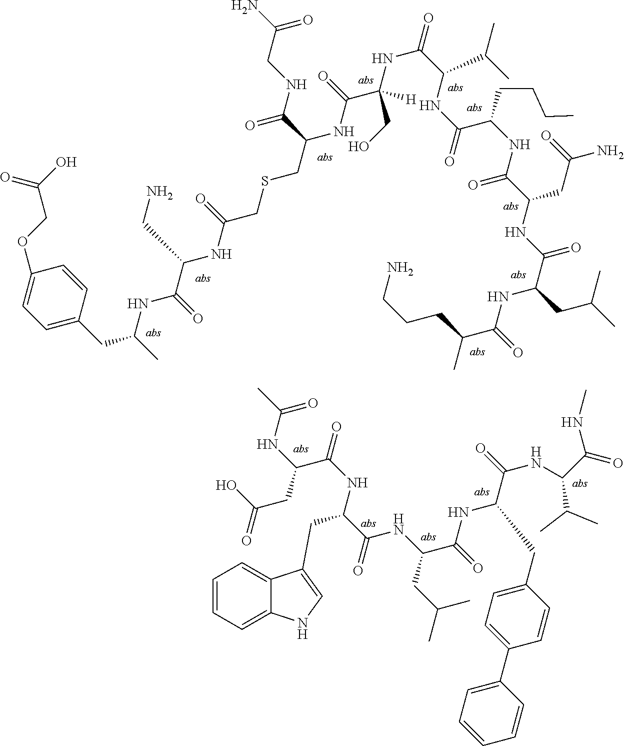

Compound 1341 was prepared on a 50 μmol scale. The yield of the product was 15.5 mg, and its estimated purity by LCMS analysis was 96.6%. Analysis condition A: Retention time=1.802 min; ESI-MS(+) m/z [M+2H]²⁺: 954.1. Analysis condition B: Retention time=1.77 min; ESI-MS(+) m/z [M+2H]²⁺: 953.6.

### Preparation of Compound 1342

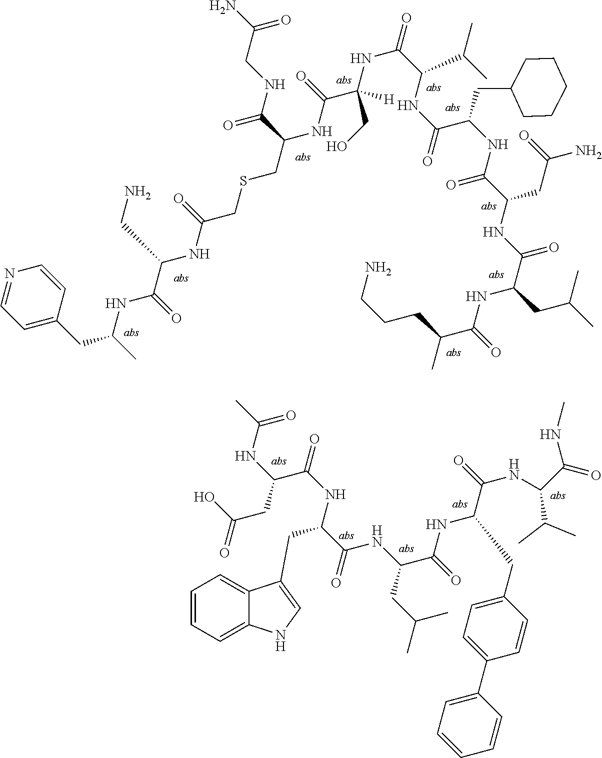

Compound 1342 was prepared on a 50 μmol scale. The yield of the product was 2.4 mg, and its estimated purity by LCMS analysis was 95.1%. Analysis condition B: Retention time=1.98 min; ESI-MS(+) m/z [M+2H]²⁺: 915.1.

### Preparation of Compound 1343

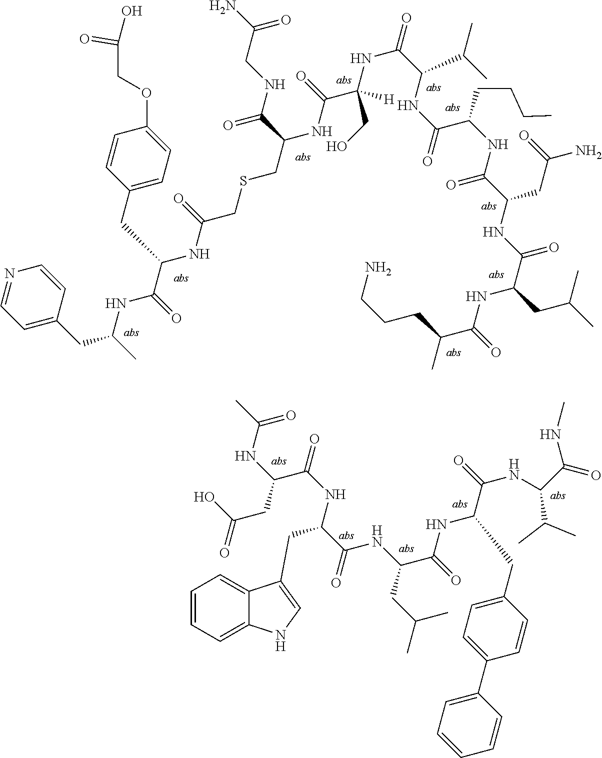

Compound 1343 was prepared on a 50 μmol scale. The yield of the product was 8.9 mg, and its estimated purity by LCMS analysis was 97.6%. Analysis condition A: Retention time=1.52, 1.59 min; ESI-MS(+) m/z [M+2H]²⁺: 983.19, 983.22.

### Preparation of Compound 1344

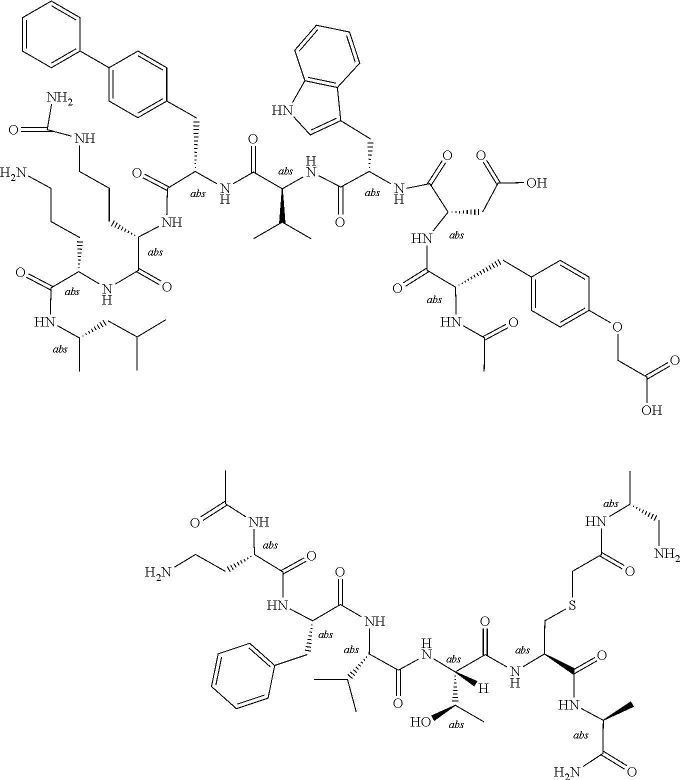 [SMILES: CC(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C)C(C)C ; CC(=O)N[C@@H](CCN)C(=O)N[C@@H](Cc1ccccc1)C(=O)N[C@H](C(=O)N[C@H](C(=O)N[C@@H](CSCC(=O)N[C@H](C)CN)C(=O)N[C@@H](C)C(N)=O)[C@@H](C)O)C(C)C]

Compound 1344 was prepared on a 50 μmol scale. The yield of the product was 23.2 mg, and its estimated purity by LCMS analysis was 100%. Analysis condition A: Retention time=1.47 min; ESI-MS(+) m/z [M+H]⁺: 1993.9.

### Preparation of Compound 1345

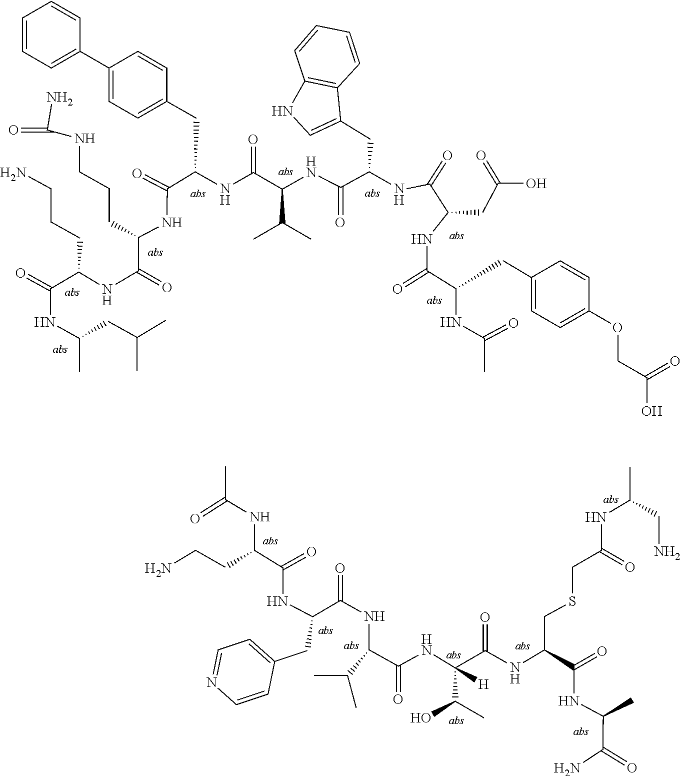 [SMILES: CC(=O)N[C@@H](Cc1ccc(OCC(=O)O)cc1)C(=O)N[C@@H](CC(=O)O)C(=O)N[C@@H](Cc1c[nH]c2ccccc12)C(=O)N[C@H](C(=O)N[C@@H](Cc1ccc(-c2ccccc2)cc1)C(=O)N[C@@H](CCCNC(N)=O)C(=O)N[C@@H](CCCN)C(=O)N[C@@H](C)CC(C)C)C(C)C ; CC(=O)N[C@@H](CCN)C(=O)N[C@@H](Cc1ccncc1)C(=O)N[C@H](C(=O)N[C@H](C(=O)N[C@@H](CSCC(=O)N[C@H](C)CN)C(=O)N[C@@H](C)C(N)=O)[C@@H](C)O)C(C)C]

Compound 1345 was prepared on a 50 μmol scale. The yield of the product was 5.1 mg, and its estimated purity by LCMS analysis was 98.1%. Analysis condition A: Retention time=1.22 min; ESI-MS(+) m/z [M+3H]³⁺: 665.9.

### Preparation of Compound 1346

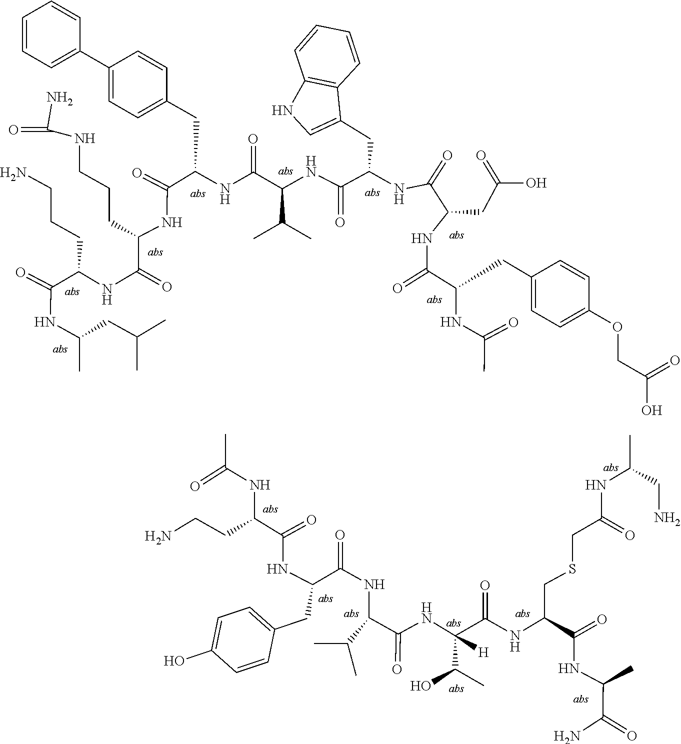

Compound 1346 was prepared on a 50 μmol scale. The yield of the product was 14.5 mg, and its estimated purity by LCMS analysis was 98.4%. Analysis condition A: Retention time=1.39 min; ESI-MS(+) m/z [M+2H]²⁺: 1006.

### Preparation of Compound 1347

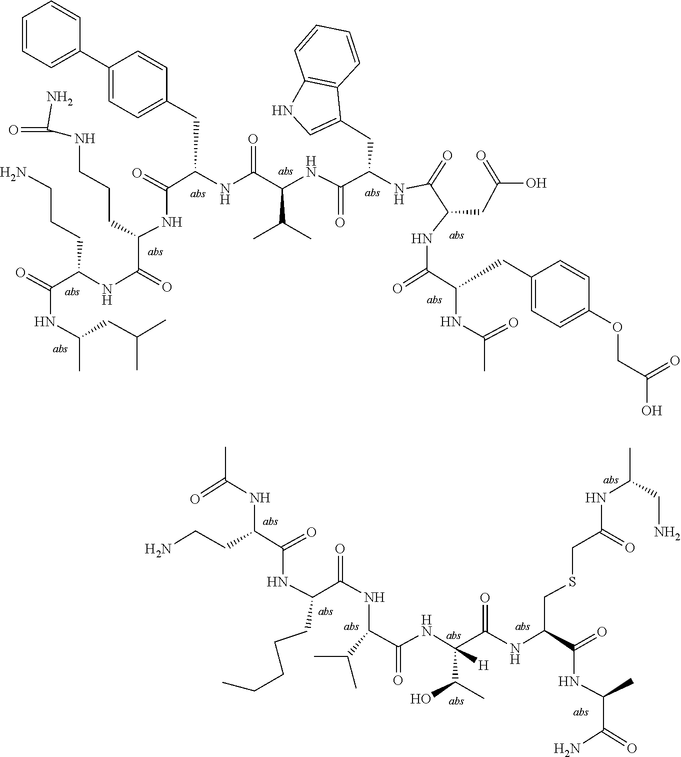

Compound 1347 was prepared on a 50 μmol scale. The yield of the product was 5.1 mg, and its estimated purity by LCMS analysis was 95.2%. Analysis condition B: Retention time=1.48 min; ESI-MS(+) m/z [M+3H]³⁺: 659.1.

### Preparation of Compound 1348

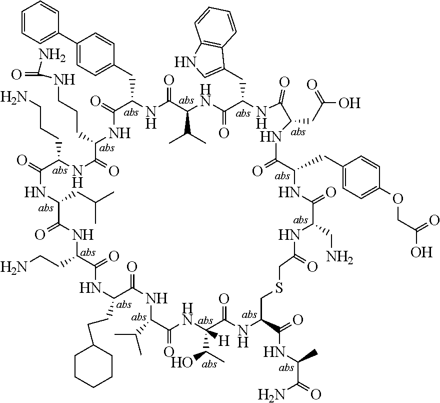 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CCC2CCCCC2)NC(=O)[C@H](CCN)NC1=O]

Compound 1348 was prepared on a 50 μmol scale. The yield of the product was 8.4 mg, and its estimated purity by LCMS analysis was 95.3%. Analysis condition A: Retention time=1.7, 1.76 min; ESI-MS(+) m/z [M+2H]²⁺, [M+3H]³⁺: 1007.86, 672.04.

### Preparation of Compound 1349

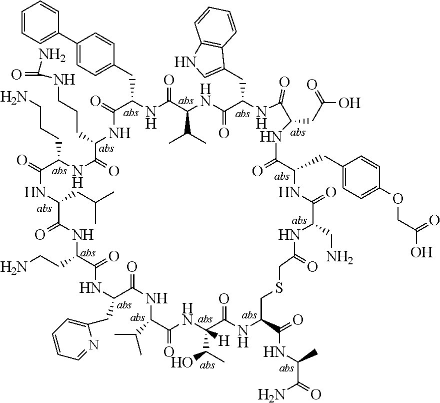 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccn2)NC(=O)[C@H](CCN)NC1=O]

Compound 1349 was prepared on a 50 μmol scale. The yield of the product was 23.1 mg, and its estimated purity by LCMS analysis was 100%. Analysis condition A: Retention time=1.41 min; ESI-MS(+) m/z [M+H]⁺: 1996.

### Preparation of Compound 1350

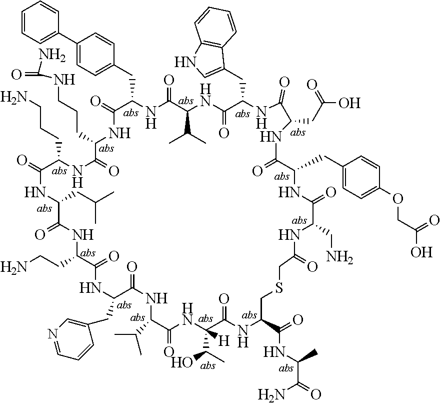 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccnc2)NC(=O)[C@H](CCN)NC1=O]

Compound 1350 was prepared on a 50 μmol scale. The yield of the product was 33.8 mg, and its estimated purity by LCMS analysis was 94.8%. Analysis condition B: Retention time=1.55 min; ESI-MS(+) m/z [M+3H]³⁺: 666.1.

### Preparation of Compound 1351

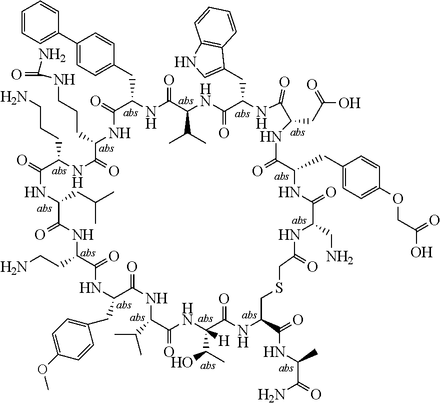 [SMILES: COc1ccc(C[C@@H]2NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC2=O)cc1]

Compound 1351 was prepared on a 50 μmol scale. The yield of the product was 18.1 mg, and its estimated purity by LCMS analysis was 95.6%. Analysis condition B: Retention time=1.89 min; ESI-MS(+) m/z [M+2H]²⁺: 1013.

### Preparation of Compound 1352

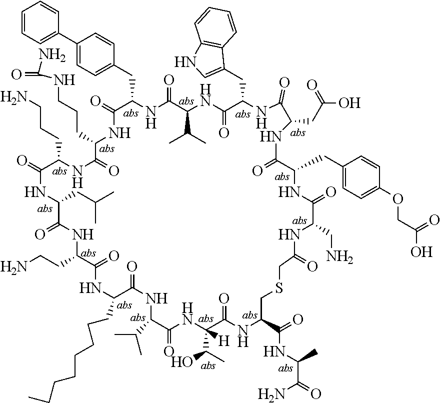 [SMILES: CCCCCCCC[C@@H]1NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1352 was prepared on a 50 μmol scale. The yield of the product was 21.9 mg, and its estimated purity by LCMS analysis was 90.8%. Analysis condition A: Retention time=1.71 min; ESI-MS(+) m/z [M+2H]²⁺: 1009.1.

### Preparation of Compound 1353

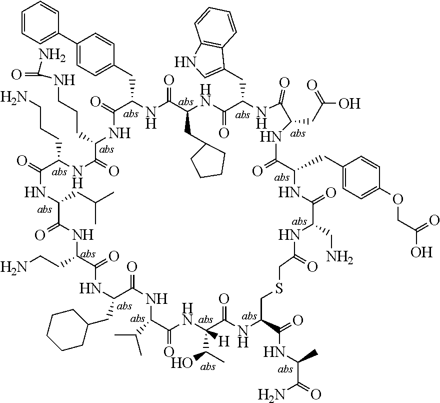 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](CC2CCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1353 was prepared on a 50 μmol scale. The yield of the product was 3 mg, and its estimated purity by LCMS analysis was 98.3%. Analysis condition B: Retention time=1.5 min; ESI-MS(+) m/z [M+2H]²⁺: 1021.3.

### Preparation of Compound 1354

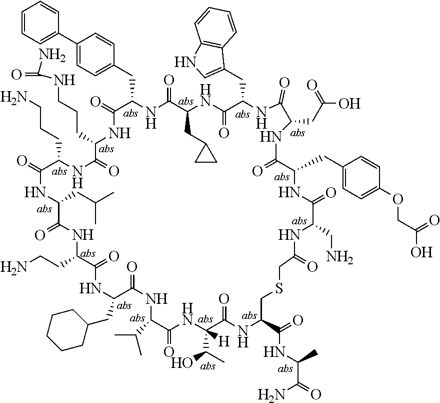 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](CC2CC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1354 was prepared on a 50 μmol scale. The yield of the product was 1.7 mg, and its estimated purity by LCMS analysis was 95.4%. Analysis condition B: Retention time=1.44 min; ESI-MS(+) m/z [M+2H]²⁺: 1007.

### Preparation of Compound 1355

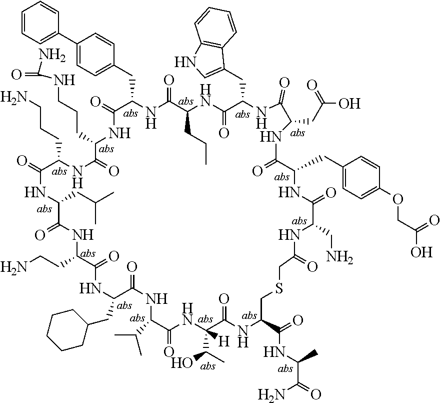 [SMILES: CCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 1355 was prepared on a 50 μmol scale. The yield of the product was 11.1 mg, and its estimated purity by LCMS analysis was 98.6%. Analysis condition A: Retention time=1.59 min; ESI-MS(+) m/z [M+2H]²⁺: 1001.1.

### Preparation of Compound 1356

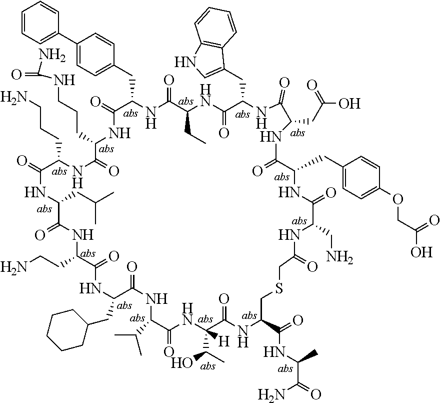 [SMILES: CC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 1356 was prepared on a 50 μmol scale. The yield of the product was 7.5 mg, and its estimated purity by LCMS analysis was 96.1%. Analysis condition A: Retention time=1.57 min; ESI-MS(+) m/z [M+H]⁺: 1987.1.

### Preparation of Compound 1357

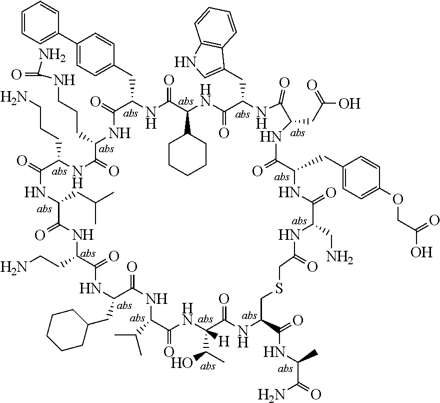 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1357 was prepared on a 50 μmol scale. The yield of the product was 13.1 mg, and its estimated purity by LCMS analysis was 94.4%. Analysis condition A: Retention time=1.67 min; ESI-MS(+) m/z [M+2H]²⁺: 1021.

### Preparation of Compound 1358

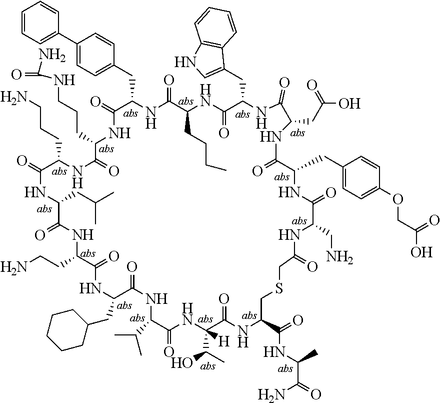 [SMILES: CCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 1358 was prepared on a 50 μmol scale. The yield of the product was 7.1 mg, and its estimated purity by LCMS analysis was 91.9%. Analysis condition A: Retention time=1.65 min; ESI-MS(+) m/z [M+2H]²⁺: 1008.1.

### Preparation of Compound 1359

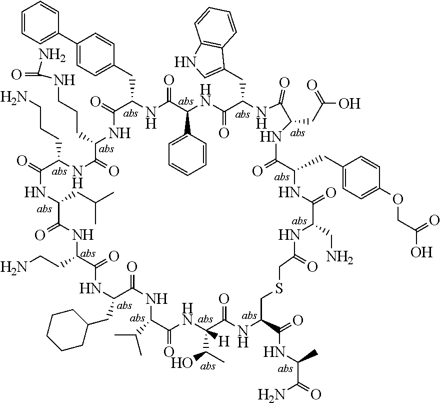 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1359 was prepared on a 50 μmol scale. The yield of the product was 4.1 mg, and its estimated purity by LCMS analysis was 99.1%. Analysis condition B: Retention time=1.46 min; ESI-MS(+) m/z [M+3H]³⁺: 679.1.

### Preparation of Compound 1360

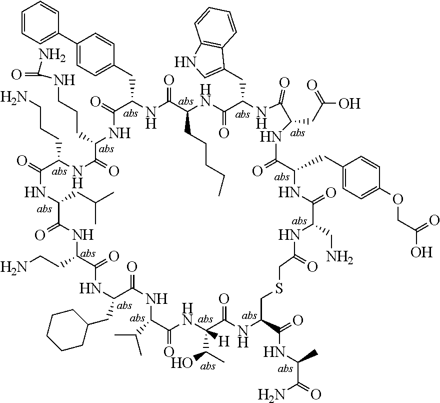 [SMILES: CCCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 1360 was prepared on a 50 μmol scale. The yield of the product was 4.4 mg, and its estimated purity by LCMS analysis was 95.6%. Analysis condition B: Retention time=1.51 min; ESI-MS(+) m/z [M+2H]²⁺: 1015.3.

### Preparation of Compound 1361

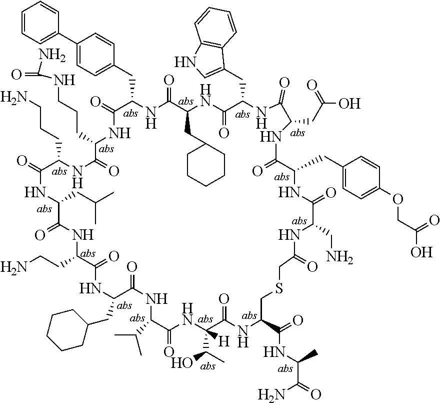 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](CC2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1361 was prepared on a 50 μmol scale. The yield of the product was 8.2 mg, and its estimated purity by LCMS analysis was 95.1%. Analysis condition B: Retention time=1.65 min; ESI-MS(+) m/z [M+2H]²⁺: 1028.2.

### Preparation of Compound 1362

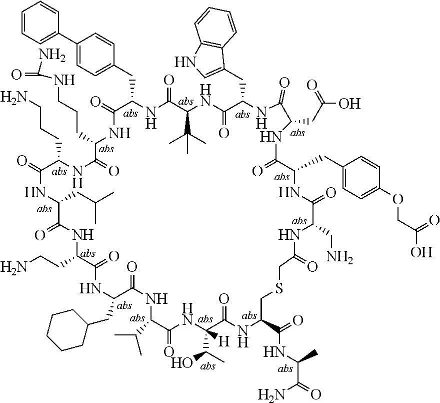 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1362 was prepared on a 50 μmol scale. The yield of the product was 6.9 mg, and its estimated purity by LCMS analysis was 91%. Analysis condition B: Retention time=1.53 min; ESI-MS(+) m/z [M+2H]²⁺: 1008.

### Preparation of Compound 1363

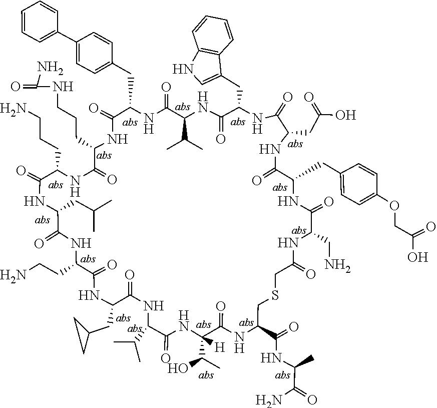 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CC2)NC(=O)[C@H](CCN)NC1=O]

Compound 1363 was prepared on a 50 μmol scale. The yield of the product was 2.5 mg, and its estimated purity by LCMS analysis was 91.8%. Analysis condition B: Retention time=1.74 min; ESI-MS(+) m/z [M+2H]²⁺: 980.2.

### Preparation of Compound 1364

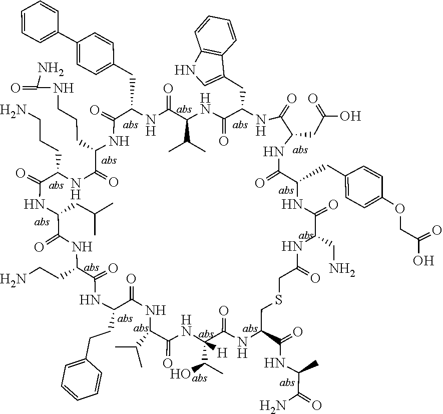 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CCc2ccccc2)NC(=O)[C@H](CCN)NC1=O]

Compound 1364 was prepared on a 50 μmol scale. The yield of the product was 4.5 mg, and its estimated purity by LCMS analysis was 95.4%. Analysis condition B: Retention time=1.46 min; ESI-MS(+) m/z [M+2H]²⁺: 1005.3.

### Preparation of Compound 1365

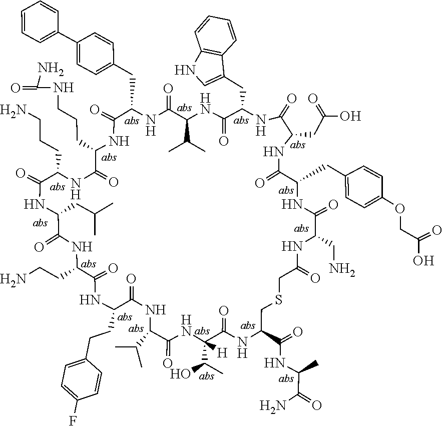 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CCc2ccc(F)cc2)NC(=O)[C@H](CCN)NC1=O]

Compound 1365 was prepared on a 50 μmol scale. The yield of the product was 12.3 mg, and its estimated purity by LCMS analysis was 96.6%. Analysis condition A: Retention time=1.34 min; ESI-MS(+) m/z [M+2H]²⁺: 1007.

### Preparation of Compound 1366

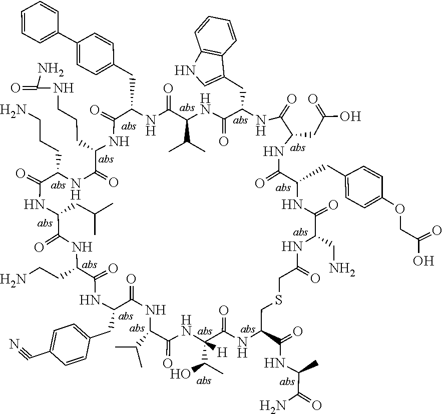 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(C#N)cc2)NC(=O)[C@H](CCN)NC1=O]

Compound 1366 was prepared on a 50 μmol scale. The yield of the product was 6.3 mg, and its estimated purity by LCMS analysis was 97.4%. Analysis condition B: Retention time=1.82 min; ESI-MS(+) m/z [M+3H]³⁺: 674.

### Preparation of Compound 1367

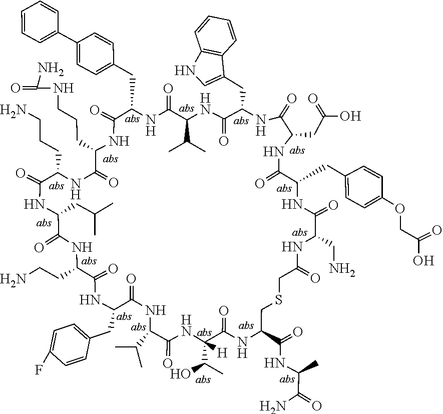 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(F)cc2)NC(=O)[C@H](CCN)NC1=O]

Compound 1367 was prepared on a 50 μmol scale. The yield of the product was 14.1 mg, and its estimated purity by LCMS analysis was 100%. Analysis condition A: Retention time=1.42 min; ESI-MS(+) m/z [M+2H]²⁺: 1007.1.

### Preparation of Compound 1368

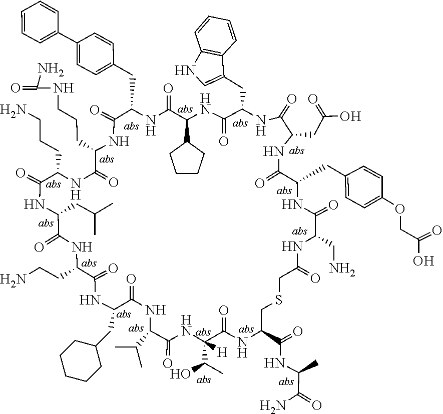 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C2CCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1368 was prepared on a 50 μmol scale. The yield of the product was 7.6 mg, and its estimated purity by LCMS analysis was 98.2%. Analysis condition A: Retention time=1.5 min; ESI-MS(+) m/z [M+2H]²⁺: 1014.

### Preparation of Compound 1369

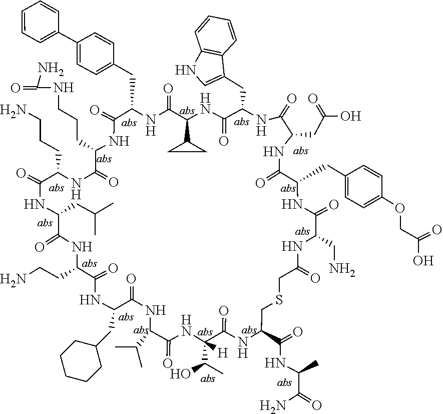 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C2CC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1369 was prepared on a 50 μmol scale. The yield of the product was 6.4 mg, and its estimated purity by LCMS analysis was 97.9%. Analysis condition B: Retention time=1.42 min; ESI-MS(+) m/z [M+3H]³⁺: 667.

### Preparation of Compound 1370

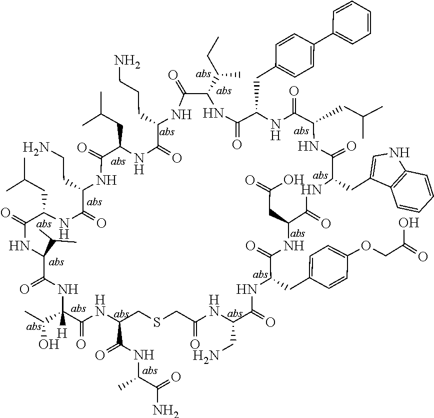 [SMILES: CC[C@H](C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1370 was prepared on a 50 μmol scale. The yield of the product was 7.6 mg, and its estimated purity by LCMS analysis was 92.2%. Analysis condition B: Retention time=1.47 min; ESI-MS(+) m/z [M+H]⁺: 1930.2.

### Preparation of Compound 1371

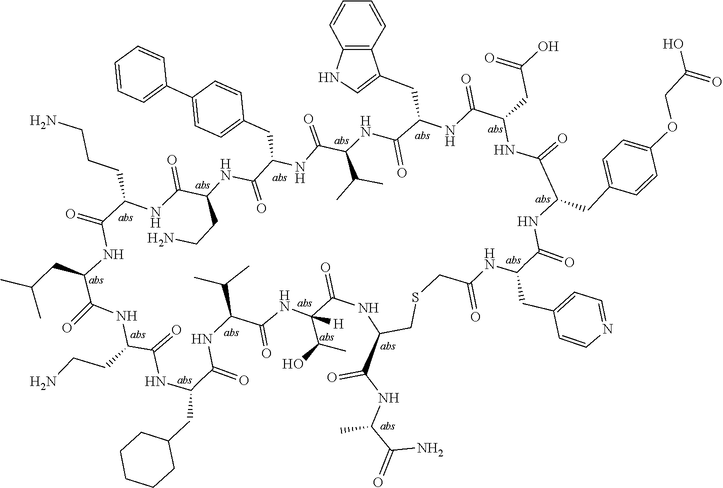 [SMILES: CC(C)C[C@H]1NC(=O)[C@H](CCCN)NC(=O)[C@H](CCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1371 was prepared on a 50 μmol scale. The yield of the product was 18.8 mg, and its estimated purity by LCMS analysis was 93.4%. Analysis condition B: Retention time=1.47 min; ESI-MS(+) m/z [M+3H]³⁺: 669.

### Preparation of Compound 1372

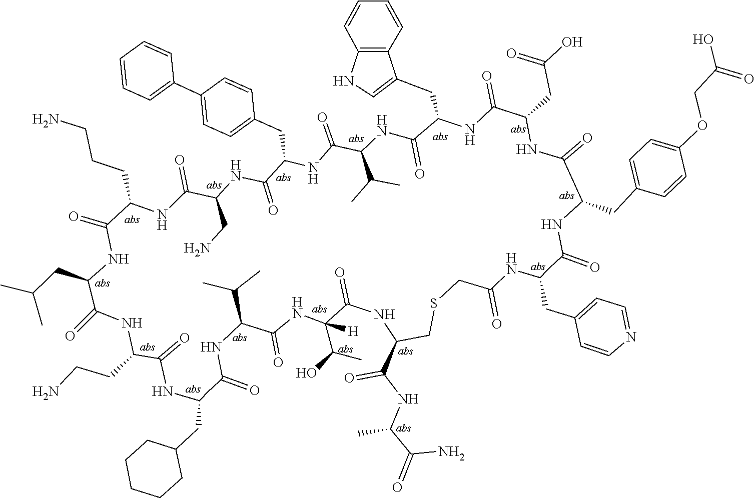 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1372 was prepared on a 50 μmol scale. The yield of the product was 8.2 mg, and its estimated purity by LCMS analysis was 91.2%. Analysis condition A: Retention time=1.56, 1.59 min; ESI-MS(+) m/z [M+H]⁺: 1991.25, 1991.25.

### Preparation of Compound 1373

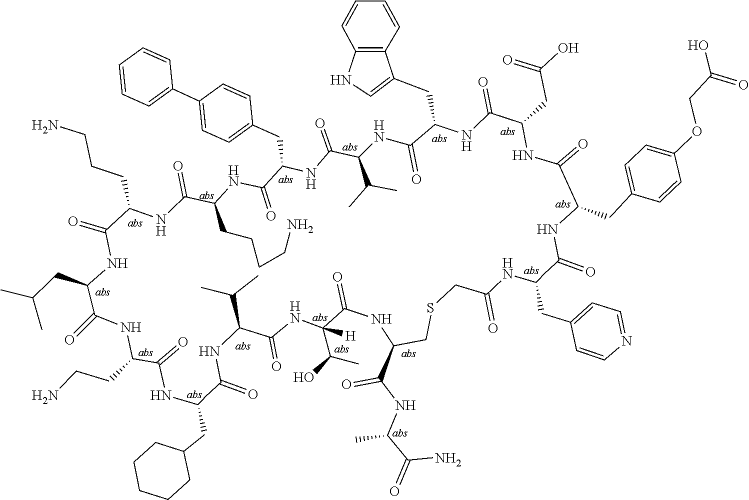 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1373 was prepared on a 50 μmol scale. The yield of the product was 24.7 mg, and its estimated purity by LCMS analysis was 95.9%. Analysis condition A: Retention time=1.55 min; ESI-MS(+) m/z [M+3H]³⁺: 678.4.

### Preparation of Compound 1374

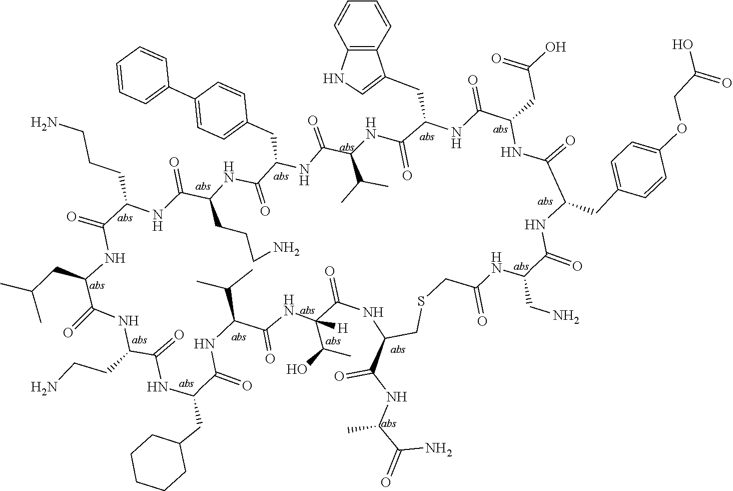 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1374 was prepared on a 50 μmol scale. The yield of the product was 13.3 mg, and its estimated purity by LCMS analysis was 98.2%. Analysis condition B: Retention time=1.46 min; ESI-MS(+) m/z [M+H]⁺: 1958.

### Preparation of Compound 1375

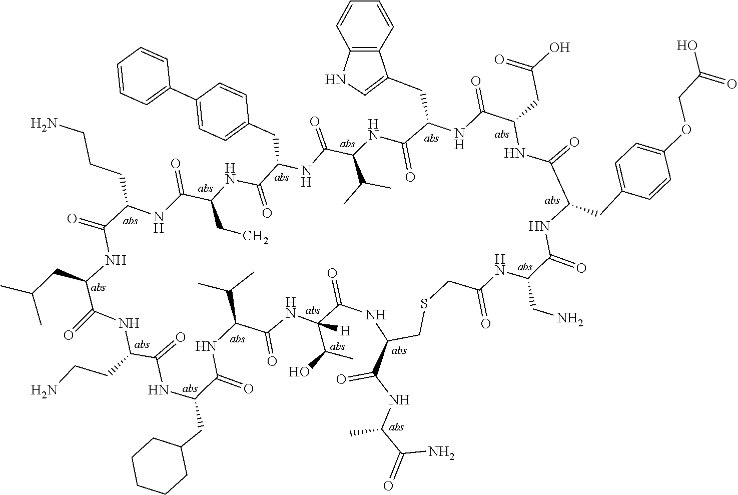 [SMILES: CC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC(=O)[C@@H](CC(C)C)NC(=O)[C@H](CCCN)NC1=O]

Compound 1375 was prepared on a 50 μmol scale. The yield of the product was 23 mg, and its estimated purity by LCMS analysis was 94.6%. Analysis condition A: Retention time=1.51 min; ESI-MS(+) m/z [M+H]⁺: 1943.1.

### Preparation of Compound 1376

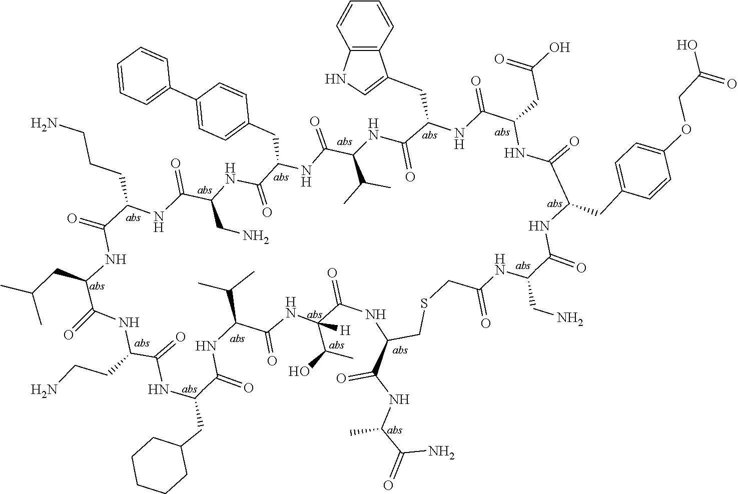 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1376 was prepared on a 50 μmol scale. The yield of the product was 11.1 mg, and its estimated purity by LCMS analysis was 92.3%. Analysis condition B: Retention time=1.4 min; ESI-MS(+) m/z [M+H]⁺: 1929.3.

### Preparation of Compound 1377

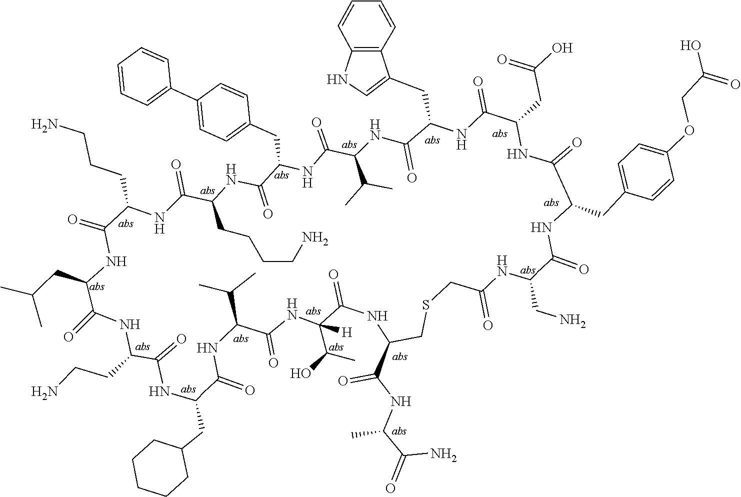 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)C[C@H](CC2CCCCC2)NC(=O)[C@H](CCN)NC1=O]

Compound 1377 was prepared on a 50 μmol scale. The yield of the product was 17.3 mg, and its estimated purity by LCMS analysis was 100%. Analysis condition B: Retention time=1.46 min; ESI-MS(+) m/z [M+H]⁺: 1971.3.

### Preparation of Compound 1378

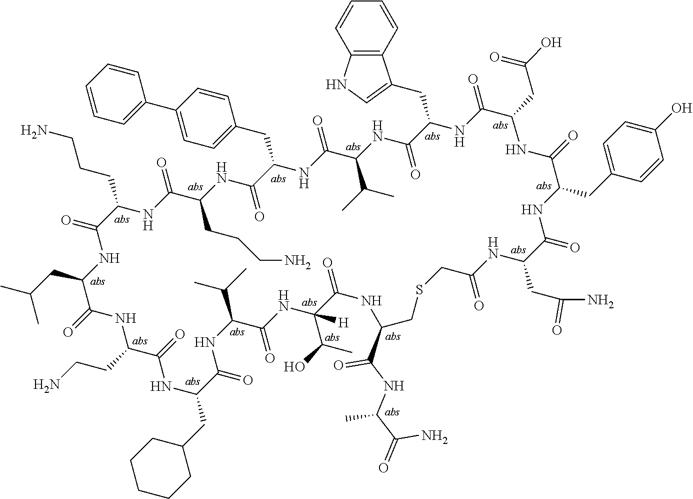 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1378 was prepared on a 50 μmol scale. The yield of the product was 17 mg, and its estimated purity by LCMS analysis was 96.8%. Analysis condition B: Retention time=1.5 min; ESI-MS(+) m/z [M+H]⁺: 1927.3.

### Preparation of Compound 1379

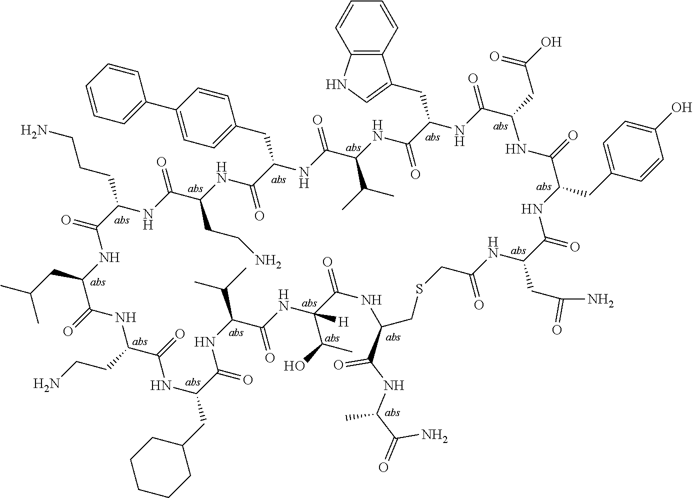 [SMILES: CC(C)C[C@H]1NC(=O)[C@H](CCCN)NC(=O)[C@H](CCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1379 was prepared on a 50 μmol scale. The yield of the product was 11.9 mg, and its estimated purity by LCMS analysis was 96.9%. Analysis condition B: Retention time=1.44 min; ESI-MS(+) m/z [M+H]⁺: 1913.1.

### Preparation of Compound 1380

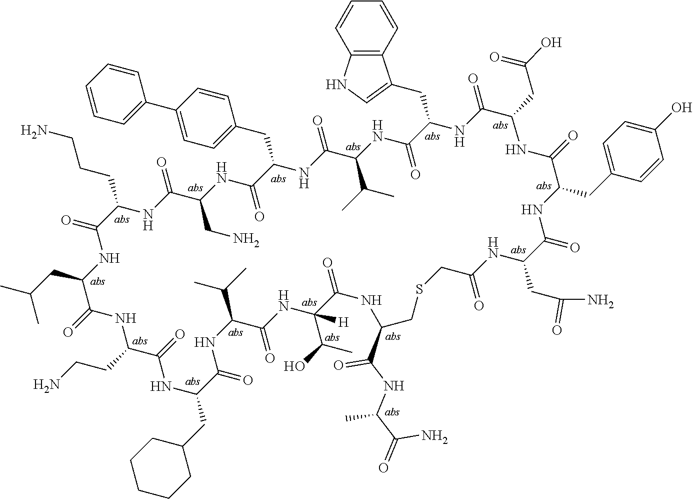 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1380 was prepared on a 50 μmol scale. The yield of the product was 17.9 mg, and its estimated purity by LCMS analysis was 94.2%. Analysis condition A: Retention time=1.63 min; ESI-MS(+) m/z [M+H]⁺: 1899.1.

### Preparation of Compound 1381

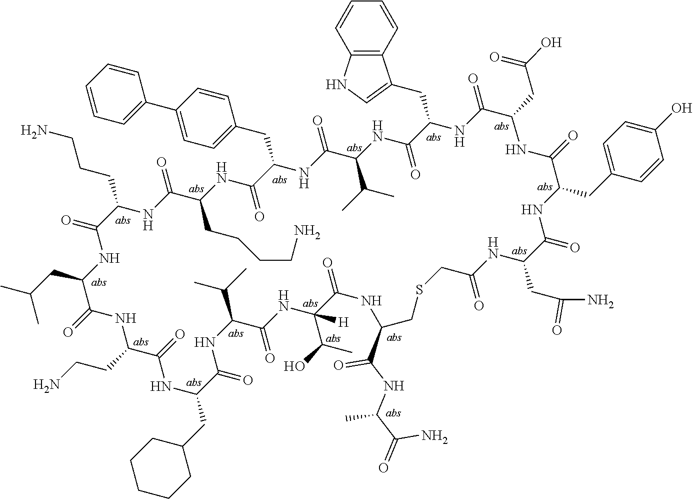 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1381 was prepared on a 50 μmol scale. The yield of the product was 17.7 mg, and its estimated purity by LCMS analysis was 96.6%. Analysis condition B: Retention time=1.45 min; ESI-MS(+) m/z [M+H]⁺: 1942.1.

### Preparation of Compound 1382

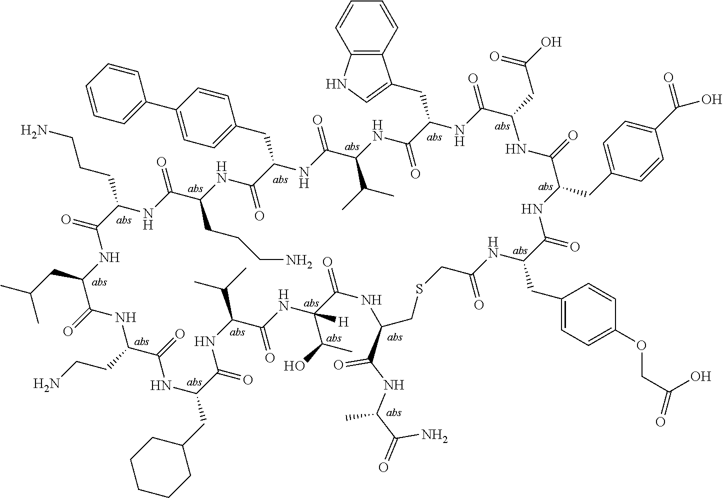 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1382 was prepared on a 50 μmol scale. The yield of the product was 12.1 mg, and its estimated purity by LCMS analysis was 95.7%. Analysis condition A: Retention time=1.56 min; ESI-MS(+) m/z [M+2H]²⁺: 1031.9.

### Preparation of Compound 1383

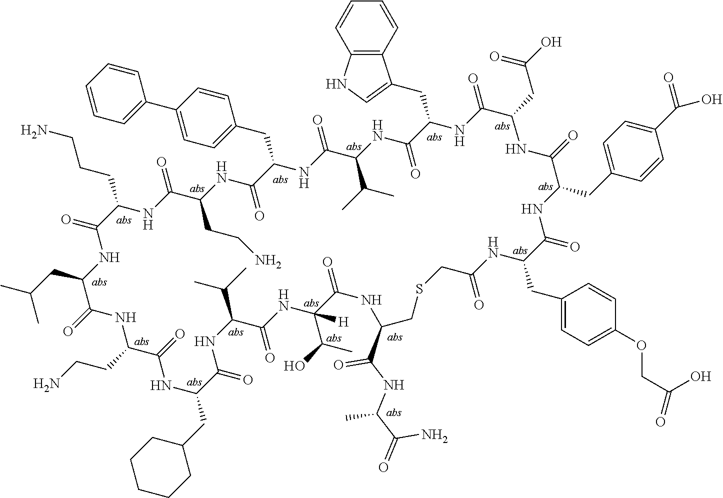 [SMILES: CC(C)C[C@H]1NC(=O)[C@H](CCCN)NC(=O)[C@H](CCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1383 was prepared on a 50 μmol scale. The yield of the product was 17.6 mg, and its estimated purity by LCMS analysis was 91.6%. Analysis condition A: Retention time=1.53 min; ESI-MS(+) m/z [M+2H]²⁺: 1024.9.

### Preparation of Compound 1384

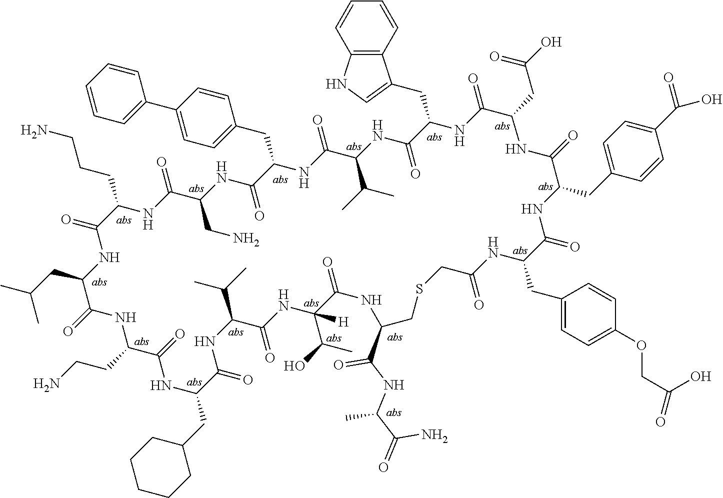 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1384 was prepared on a 50 μmol scale. The yield of the product was 15.3 mg, and its estimated purity by LCMS analysis was 91.6%. Analysis condition A: Retention time=1.52 min; ESI-MS(+) m/z [M+2H]²⁺: 1017.9.

### Preparation of Compound 1385

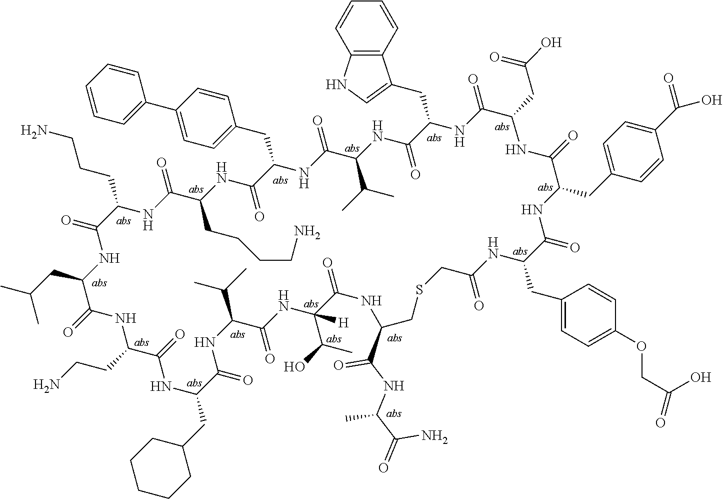 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1385 was prepared on a 50 μmol scale. The yield of the product was 14.6 mg, and its estimated purity by LCMS analysis was 94.6%. Analysis condition B: Retention time=1.58 min; ESI-MS(+) m/z [M+2H]²⁺: 1039.

### Preparation of Compound 1386

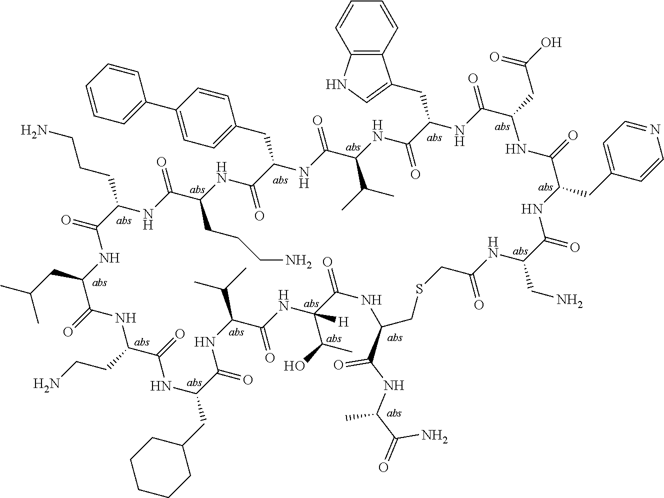 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1386 was prepared on a 50 μmol scale. The yield of the product was 25 mg, and its estimated purity by LCMS analysis was 95.8%. Analysis condition B: Retention time=1.21 min; ESI-MS(+) m/z [M+3H]³⁺: 628.9.

### Preparation of Compound 1387

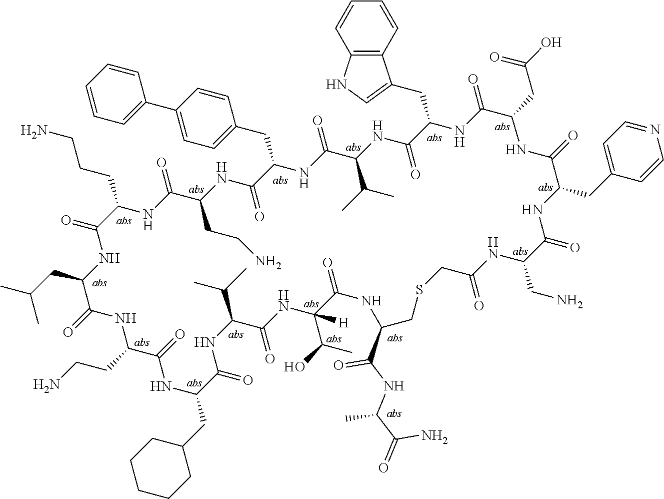 [SMILES: CC(C)C[C@H]1NC(=O)[C@H](CCCN)NC(=O)[C@H](CCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1387 was prepared on a 50 μmol scale. The yield of the product was 16.2 mg, and its estimated purity by LCMS analysis was 95.9%. Analysis condition B: Retention time=1.36 min; ESI-MS(+) m/z [M+H]⁺: 1870.3.

### Preparation of Compound 1388

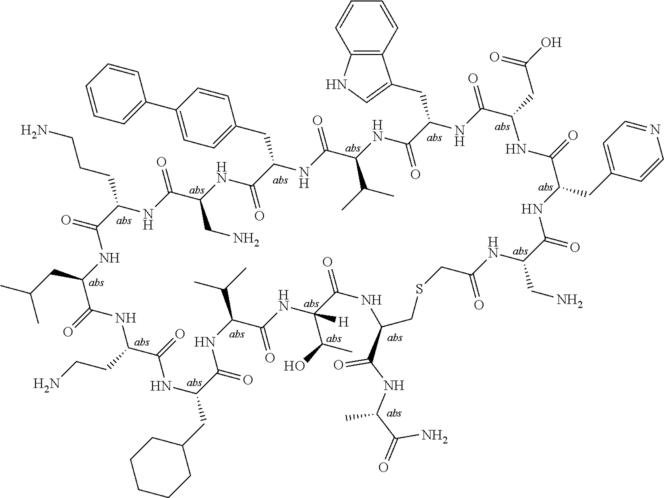 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1388 was prepared on a 50 μmol scale. The yield of the product was 18.1 mg, and its estimated purity by LCMS analysis was 90.9%. Analysis condition A: Retention time=1.44 min; ESI-MS(+) m/z [M+2H]²⁺: 929.

### Preparation of Compound 1389

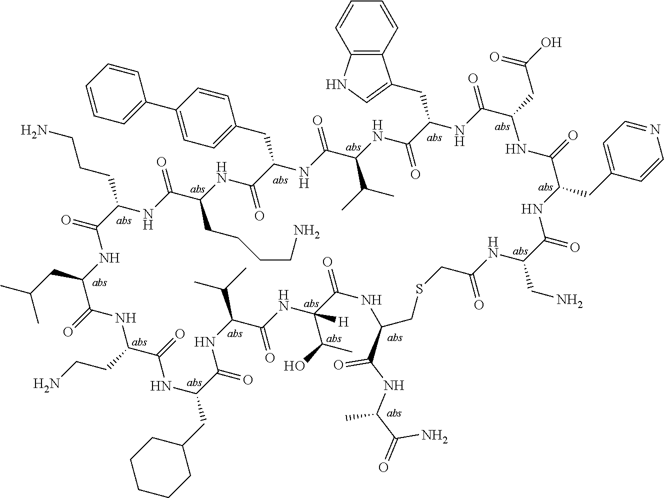 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1389 was prepared on a 50 μmol scale. The yield of the product was 9.3 mg, and its estimated purity by LCMS analysis was 100%. Analysis condition A: Retention time=1.5 min; ESI-MS(+) m/z [M+2H]²⁺: 950.

### Preparation of Compound 1390

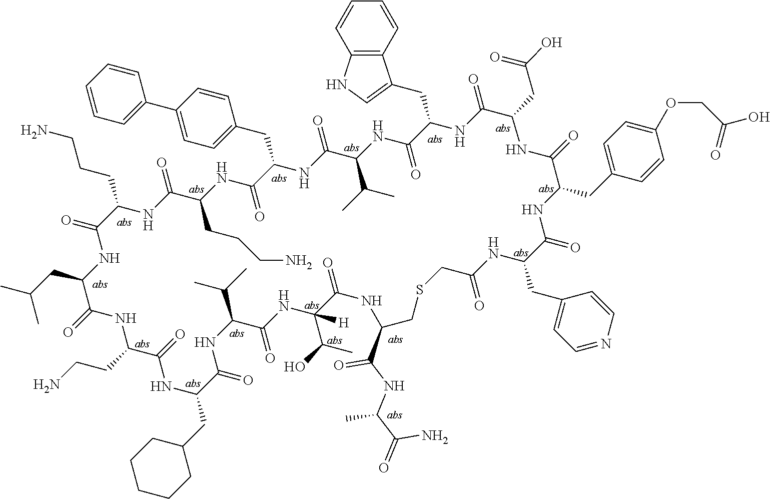 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1390 was prepared on a 50 μmol scale. The yield of the product was 23.9 mg, and its estimated purity by LCMS analysis was 95.8%. Analysis condition A: Retention time=1.55 min; ESI-MS(+) m/z [M+3H]³⁺: 674.2.

### Preparation of Compound 1391

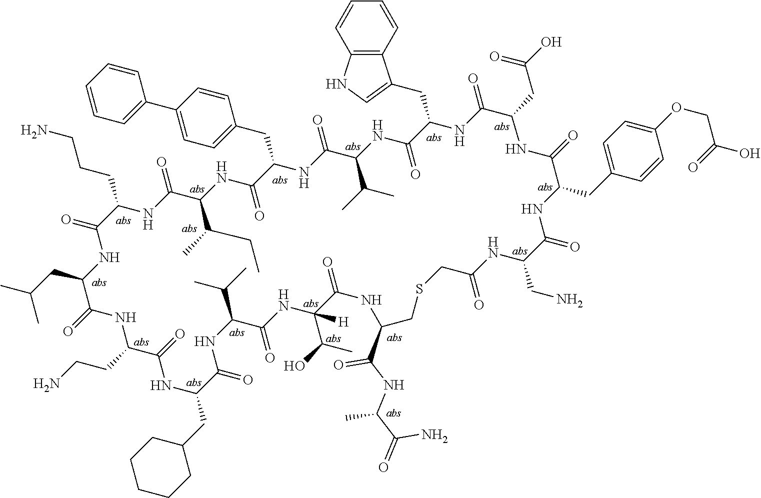 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1391 was prepared on a 50 μmol scale. The yield of the product was 9.8 mg, and its estimated purity by LCMS analysis was 94.2%. Analysis condition A: Retention time=1.66, 1.71 min; ESI-MS(+) m/z [M+H]⁺: 1957.05, 1956.3.

### Preparation of Compound 1392

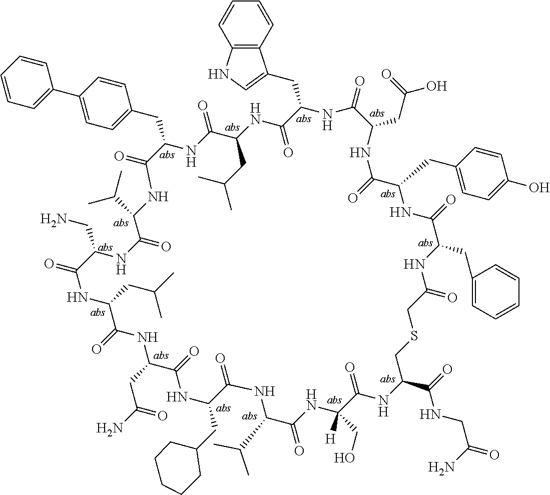 [SMILES: CC(C)C[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1392 was prepared on a 50 μmol scale. The yield of the product was 27.1 mg, and its estimated purity by LCMS analysis was 97.9%. Analysis condition B: Retention time=2 min; ESI-MS(+) m/z [M+H]⁺: 1917.3.

### Preparation of Compound 1393

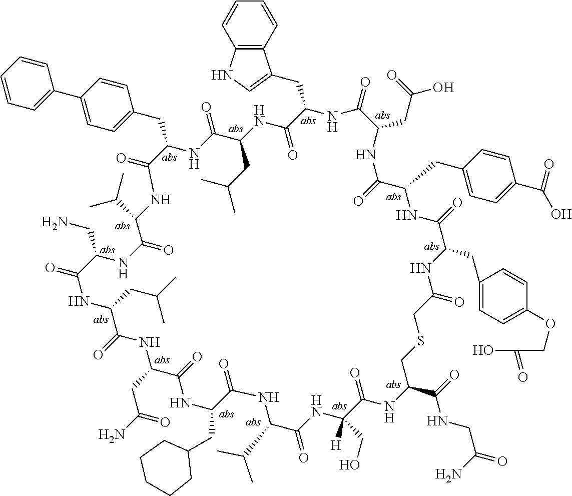 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1393 was prepared on a 50 μmol scale. The yield of the product was 13.8 mg, and its estimated purity by LCMS analysis was 91.7%. Analysis condition A: Retention time=1.76 min; ESI-MS(+) m/z [M+2H]²⁺: 1010.1.

### Preparation of Compound 1394

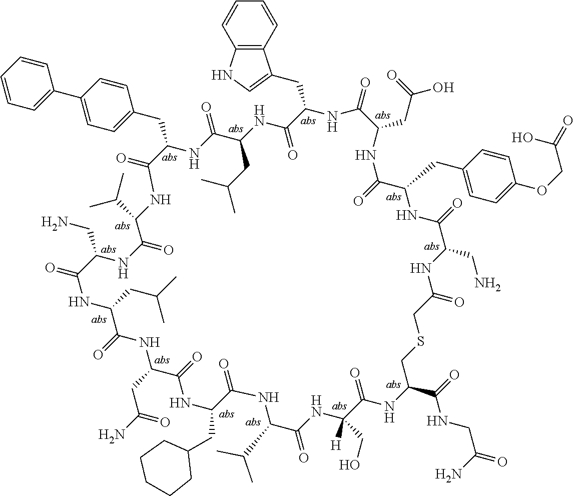 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1394 was prepared on a 50 μmol scale. The yield of the product was 23.7 mg, and its estimated purity by LCMS analysis was 94.5%. Analysis condition A: Retention time=1.7 min; ESI-MS(+) m/z [M+2H]²⁺: 958.1.

### Preparation of Compound 1395

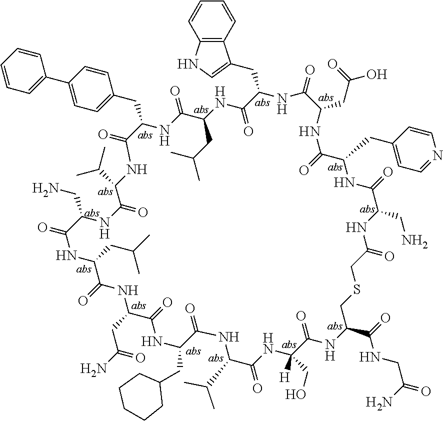 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1395 was prepared on a 50 μmol scale. The yield of the product was 13.3 mg, and its estimated purity by LCMS analysis was 91.3%. Analysis condition B: Retention time=1.62 min; ESI-MS(+) m/z [M+2H]²⁺: 1841.

### Preparation of Compound 1396

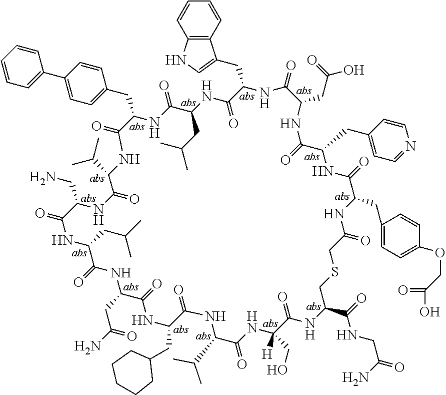 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC1=O]

Compound 1396 was prepared on a 50 μmol scale. The yield of the product was 34.1 mg, and its estimated purity by LCMS analysis was 90.2%. Analysis condition A: Retention time=1.72 min; ESI-MS(+) m/z [M+2H]²⁺: 989.2.

### Preparation of Compound 1397

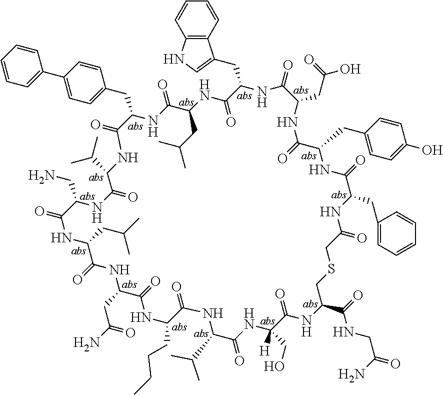 [SMILES: CCCC[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC1=O]

Compound 1397 was prepared on a 50 μmol scale. The yield of the product was 19.9 mg, and its estimated purity by LCMS analysis was 92.8%. Analysis condition B: Retention time=1.9 min; ESI-MS(+) m/z [M+H]⁺: 1877.

### Preparation of Compound 1398

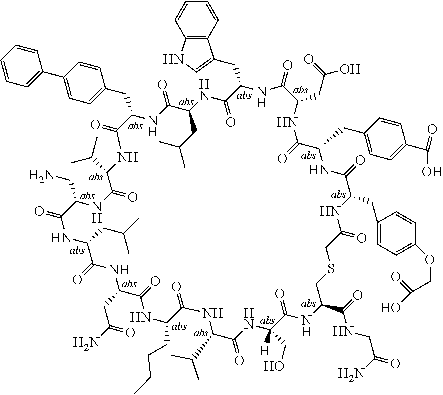 [SMILES: CCCC[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1398 was prepared on a 50 μmol scale. The yield of the product was 11.9 mg, and its estimated purity by LCMS analysis was 97.4%. Analysis condition A: Retention time=1.49 min; ESI-MS(+) m/z [M+2H]²⁺: 990.4.

### Preparation of Compound 1399

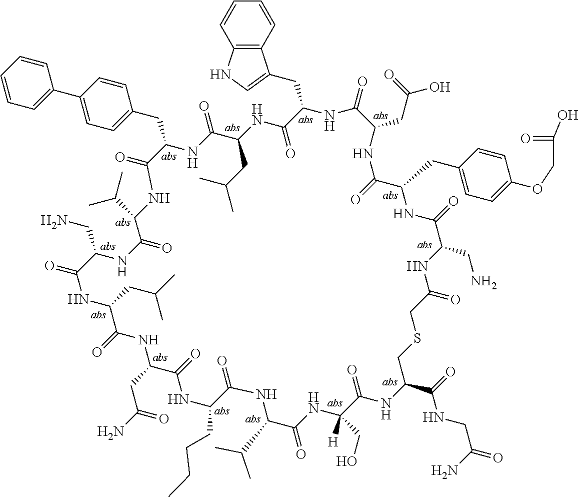 [SMILES: CCCC[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC1=O]

Compound 1399 was prepared on a 50 μmol scale. The yield of the product was 12.9 mg, and its estimated purity by LCMS analysis was 85.9%. Analysis condition B: Retention time=1.64 min; ESI-MS(+) m/z [M+H]⁺: 1874.2.

### Preparation of Compound 1400

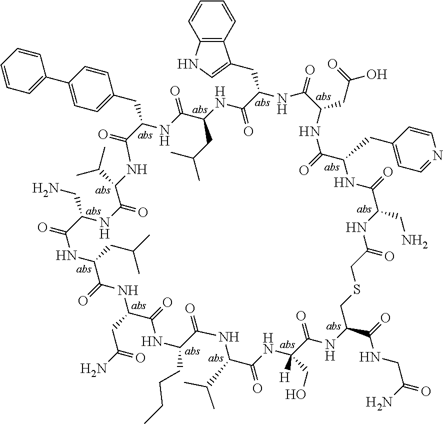 [SMILES: CCCC[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](CO)NC(=O)[C@H](C(C)C)NC1=O]

Compound 1400 was prepared on a 50 μmol scale. The yield of the product was 14.7 mg, and its estimated purity by LCMS analysis was 95.7%. Analysis condition A: Retention time=1.71 min; ESI-MS(+) m/z [M+H]⁺: 1800.6.

### Preparation of Compound 1401

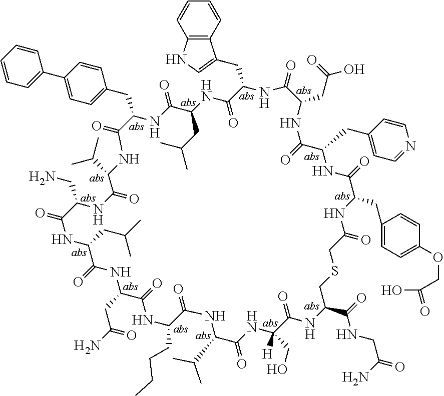 [SMILES: CCCC[C@@H]1NC(=O)[C@H](CC(N)=O)NC(=O)[C@@H](CC(C)C)NC(=O)[C@H](CN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1401 was prepared on a 50 μmol scale. The yield of the product was 11.1 mg, and its estimated purity by LCMS analysis was 93.9%. Analysis condition A: Retention time=1.68 min; ESI-MS(+) m/z [M+H]⁺: 1936.1.

### Preparation of Compound 1402

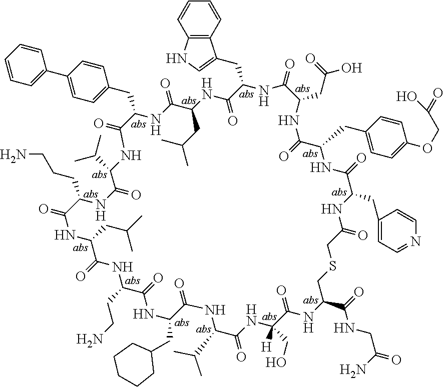 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1402 was prepared on a 50 μmol scale. The yield of the product was 25.2 mg, and its estimated purity by LCMS analysis was 85.1%. Analysis condition B: Retention time=1.52 min; ESI-MS(+) m/z [M+H]⁺: 1990.3.

### Preparation of Compound 1403

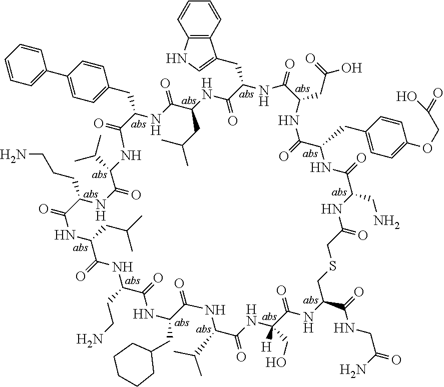 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1403 was prepared on a 50 μmol scale. The yield of the product was 10.2 mg, and its estimated purity by LCMS analysis was 94.3%. Analysis condition A: Retention time=1.64 min; ESI-MS(+) m/z [M+H]⁺: 1928.2.

### Preparation of Compound 1404

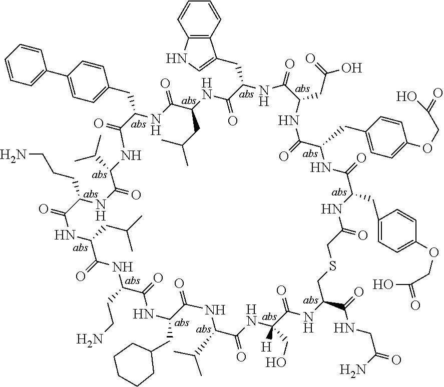 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1404 was prepared on a 50 μmol scale. The yield of the product was 22.2 mg, and its estimated purity by LCMS analysis was 93.8%. Analysis condition B: Retention time=1.72 min; ESI-MS(+) m/z [M+2H]²⁺: 1032.1.

### Preparation of Compound 1405

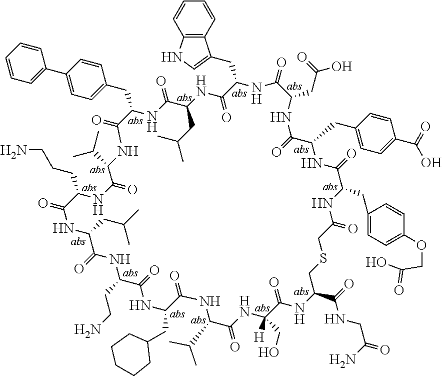 [SMILES: CC(C)C[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1405 was prepared on a 50 μmol scale. The yield of the product was 18.9 mg, and its estimated purity by LCMS analysis was 98.1%. Analysis condition A: Retention time=1.65 min; ESI-MS(+) m/z [M+2H]²⁺: 1017.2.

### Preparation of Compound 1406

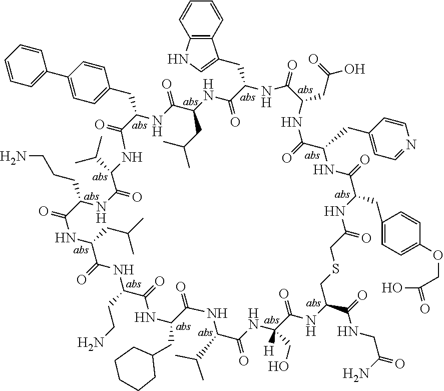 [SMILES: CC(C)C[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1406 was prepared on a 50 μmol scale. The yield of the product was 34.4 mg, and its estimated purity by LCMS analysis was 91.1%. Analysis condition A: Retention time=1.89 min; ESI-MS(+) m/z [M+2H]²⁺: 996.1.

### Preparation of Compound 1407

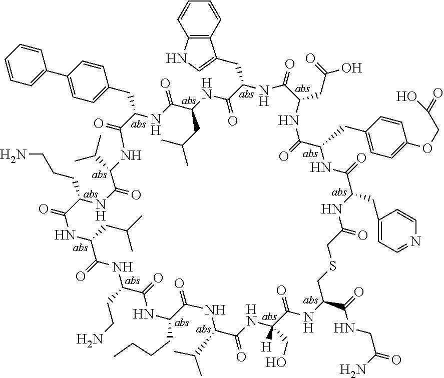 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)NCC(N)=O)NC(=O)[C@H](CO)NC(=O)[C@H](C(C)C)NC1=O]

Compound 1407 was prepared on a 50 μmol scale. The yield of the product was 11.3 mg, and its estimated purity by LCMS analysis was 92.5%. Analysis condition B: Retention time=1.46 min; ESI-MS(+) m/z [M+H]⁺: 1951.

### Preparation of Compound 1408

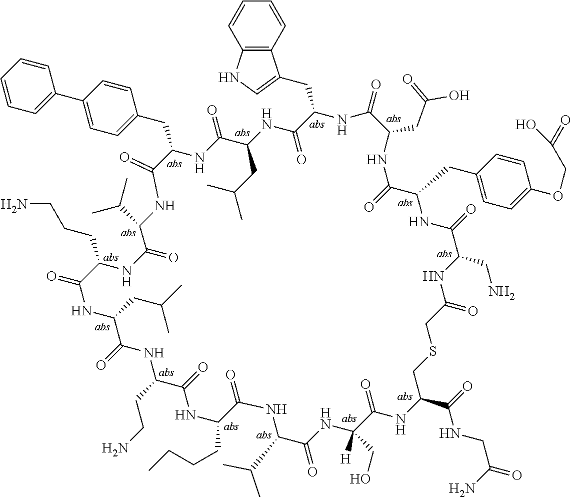 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC1=O]

Compound 1408 was prepared on a 50 μmol scale. The yield of the product was 9.7 mg, and its estimated purity by LCMS analysis was 91.3%. Analysis condition B: Retention time=1.52 min; ESI-MS(+) m/z [M+H]⁺: 1888.3.

### Preparation of Compound 1409

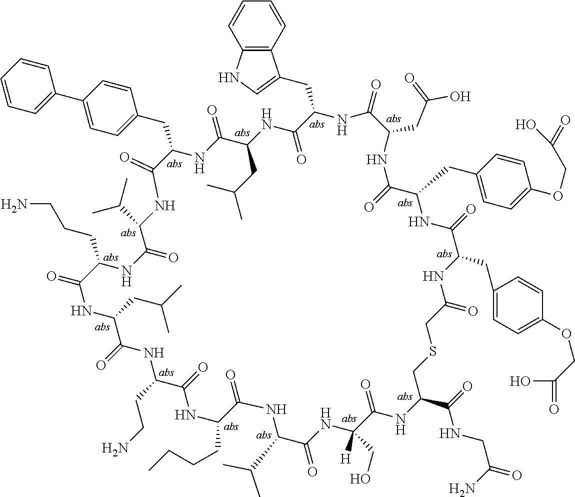 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1409 was prepared on a 50 μmol scale. The yield of the product was 5.9 mg, and its estimated purity by LCMS analysis was 92.9%. Analysis condition A: Retention time=1.62 min; ESI-MS(+) m/z [M+2H]²⁺: 1012.1.

### Preparation of Compound 1410

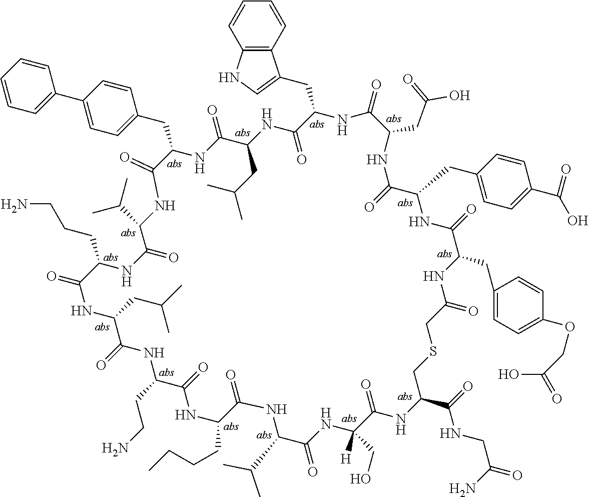 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1410 was prepared on a 50 μmol scale. The yield of the product was 11.2 mg, and its estimated purity by LCMS analysis was 100%. Analysis condition A: Retention time=1.52 min; ESI-MS(+) m/z [M+2H]²⁺: 997.5.

### Preparation of Compound 1411

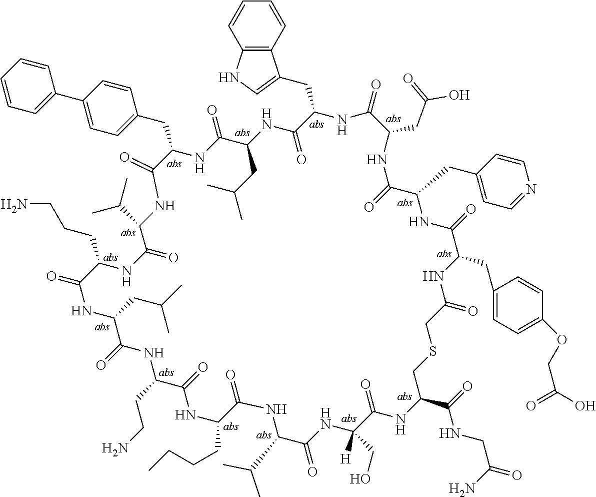 [SMILES: CCCC[C@@H]1NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC1=O]

Compound 1411 was prepared on a 50 μmol scale. The yield of the product was 7.9 mg, and its estimated purity by LCMS analysis was 98.9%. Analysis condition A: Retention time=1.82 min; ESI-MS(+) m/z [M+H]⁺: 1949.8.

### Preparation of Compound 1412

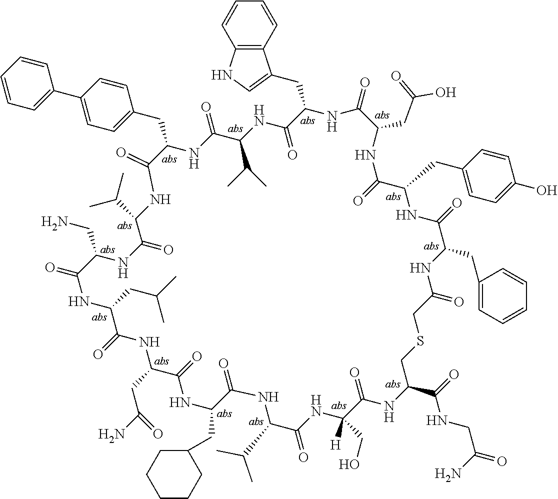 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccccc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC1=O]

Compound 1412 was prepared on a 50 μmol scale. The yield of the product was 39.7 mg, and its estimated purity by LCMS analysis was 94.4%. Analysis condition A: Retention time=1.94 min; ESI-MS(+) m/z [M+H]⁺: 1903.

### Preparation of Compound 1413

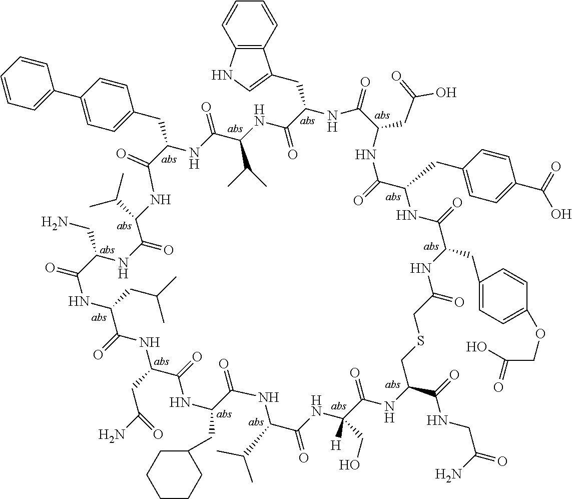 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC1=O]

Compound 1413 was prepared on a 50 μmol scale. The yield of the product was 12.8 mg, and its estimated purity by LCMS analysis was 92%. Analysis condition A: Retention time=1.61 min; ESI-MS(+) m/z [M+2H]²⁺: 1003.1.

### Preparation of Compound 1414

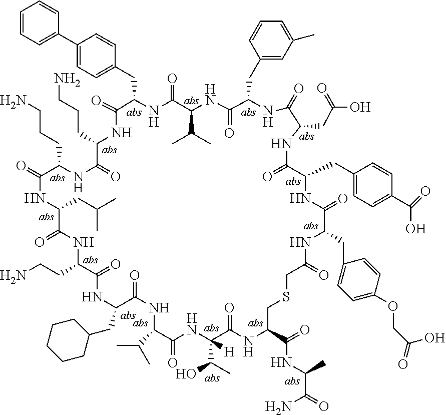 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](C(C)C)NC2=O)c1]

Compound 1414 was prepared on a 50 μmol scale. The yield of the product was 30.4 mg, and its estimated purity by LCMS analysis was 91.5%. Analysis condition A: Retention time=1.67 min; ESI-MS(+) m/z [M+2H]²⁺: 1019.1.

### Preparation of Compound 1415

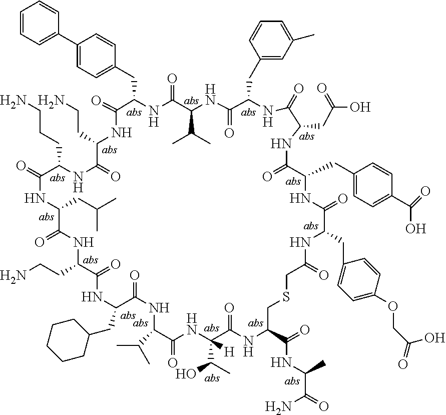 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCN)NC(=O)[C@H](Cc3ccc(-c4ccccc4)cc3)NC(=O)[C@H](C(C)C)NC2=O)c1]

Compound 1415 was prepared on a 50 μmol scale. The yield of the product was 24.8 mg, and its estimated purity by LCMS analysis was 93.1%. Analysis condition A: Retention time=1.56 min; ESI-MS(+) m/z [M+3H]³⁺: 675.4.

### Preparation of Compound 1416

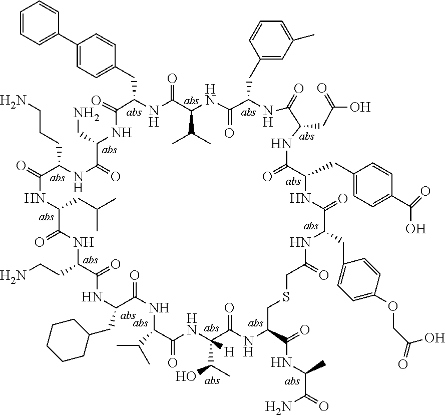 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CN)NC(=O)[C@H](Cc3ccc(-c4ccccc4)cc3)NC(=O)[C@H](C(C)C)NC2=O)c1]

Compound 1416 was prepared on a 50 μmol scale. The yield of the product was 13.6 mg, and its estimated purity by LCMS analysis was 96.1%. Analysis condition A: Retention time=1.51 min; ESI-MS(+) m/z [M+3H]³⁺: 670.9.

### Preparation of Compound 1417

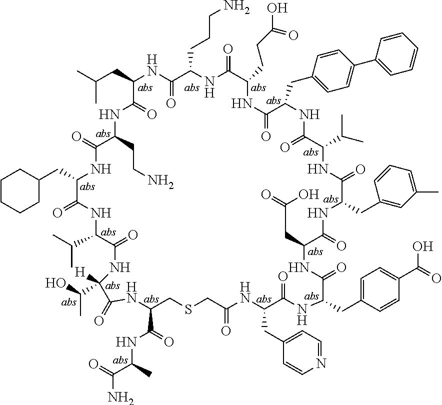 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1417 was prepared on a 50 μmol scale. The yield of the product was 13 mg, and its estimated purity by LCMS analysis was 93.6%. Analysis condition B: Retention time=1.55 min; ESI-MS(+) m/z [M+H]⁺: 1979.2.

### Preparation of Compound 1418

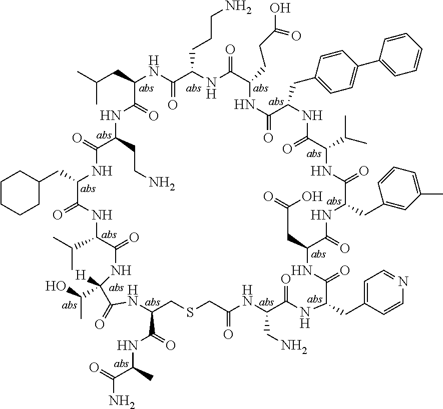 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1418 was prepared on a 50 μmol scale. The yield of the product was 9.4 mg, and its estimated purity by LCMS analysis was 93.4%. Analysis condition B: Retention time=1.46 min; ESI-MS(+) m/z [M+H]⁺: 1873.6.

### Preparation of Compound 1419

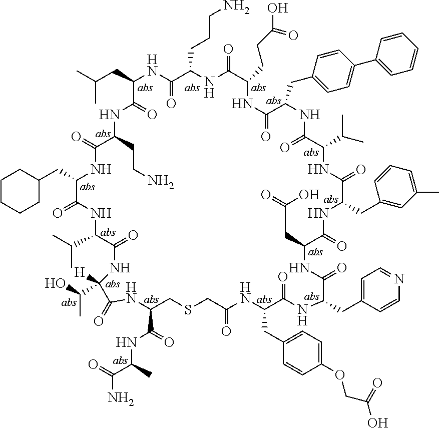 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1419 was prepared on a 50 μmol scale. The yield of the product was 8 mg, and its estimated purity by LCMS analysis was 91.7%. Analysis condition B: Retention time=1.54 min; ESI-MS(+) m/z [M+3H]³⁺: 670.9.

### Preparation of Compound 1420

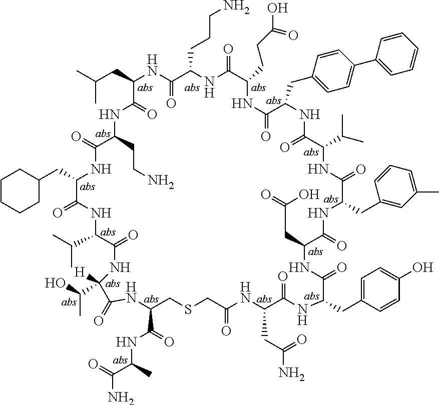 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1420 was prepared on a 50 μmol scale. The yield of the product was 10.5 mg, and its estimated purity by LCMS analysis was 90.4%. Analysis condition B: Retention time=1.64 min; ESI-MS(+) m/z [M+2H]²⁺: 959.

### Preparation of Compound 1421

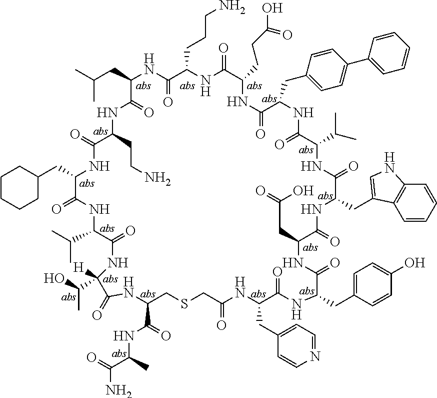 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1421 was prepared on a 50 μmol scale. The yield of the product was 19.8 mg, and its estimated purity by LCMS analysis was 99%. Analysis condition B: Retention time=1.51 min; ESI-MS(+) m/z [M+2H]²⁺: 1003.1.

### Preparation of Compound 1422

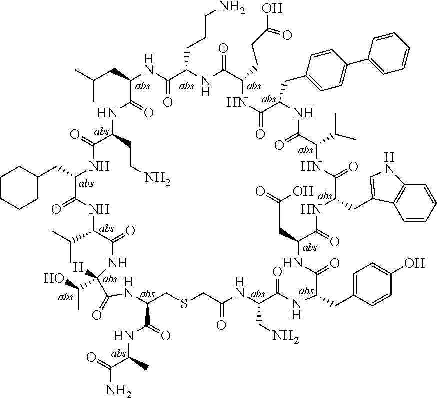 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1422 was prepared on a 50 μmol scale. The yield of the product was 12.2 mg, and its estimated purity by LCMS analysis was 99.4%. Analysis condition B: Retention time=1.3 min; ESI-MS(+) m/z [M+3H]³⁺: 634.2.

### Preparation of Compound 1423

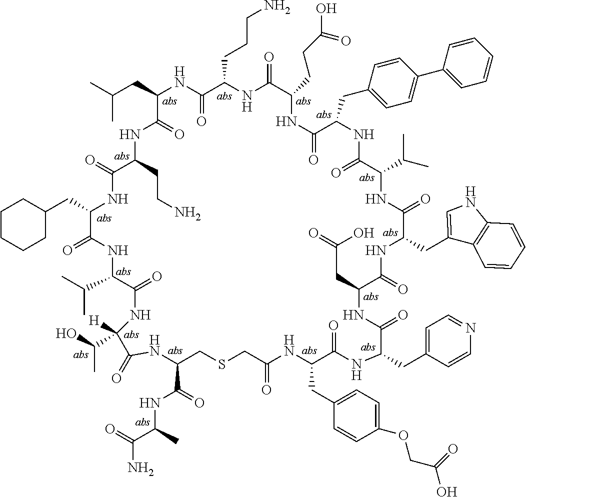 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1423 was prepared on a 50 μmol scale. The yield of the product was 13 mg, and its estimated purity by LCMS analysis was 96%. Analysis condition B: Retention time=1.5 min; ESI-MS(+) m/z [M+2H]²⁺: 1018.

### Preparation of Compound 1424

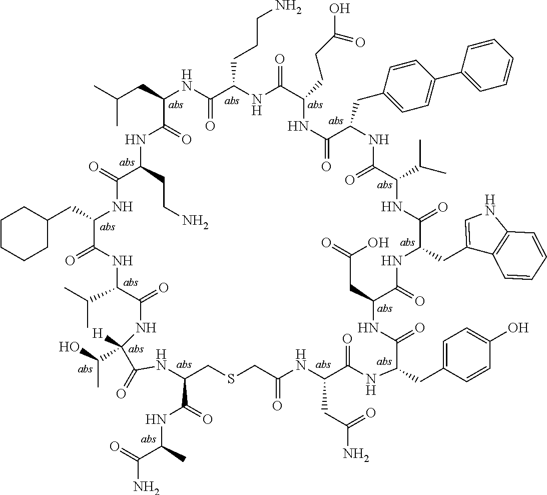 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1424 was prepared on a 50 μmol scale. The yield of the product was 17.5 mg, and its estimated purity by LCMS analysis was 97.2%. Analysis condition B: Retention time=1.6 min; ESI-MS(+) m/z [M+H]⁺: 1943.

### Preparation of Compound 1425

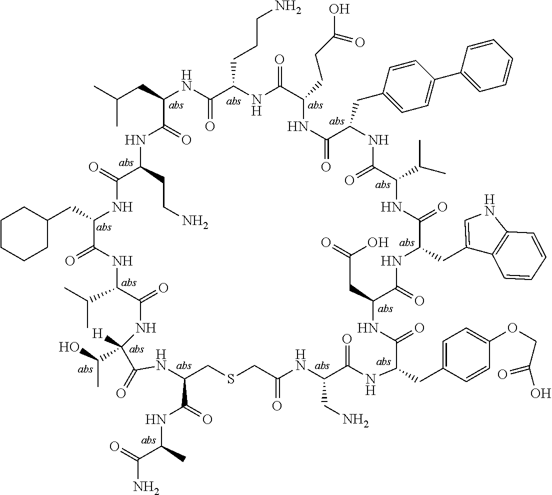 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1425 was prepared on a 50 μmol scale. The yield of the product was 11.9 mg, and its estimated purity by LCMS analysis was 90.5%. Analysis condition B: Retention time=1.41 min; ESI-MS(+) m/z [M+3H]³⁺: 658.3.

### Preparation of Compound 1426

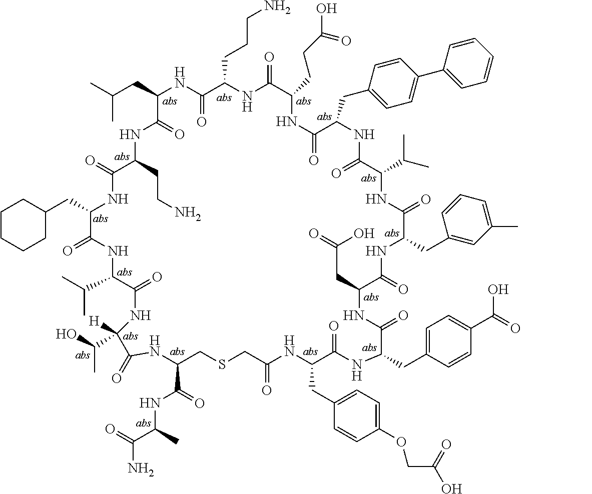 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1426 was prepared on a 50 μmol scale. The yield of the product was 1.8 mg, and its estimated purity by LCMS analysis was 94.1%. Analysis condition B: Retention time=1.7 min; ESI-MS(+) m/z [M+2H]²⁺: 1026.5.

### Preparation of Compound 1427

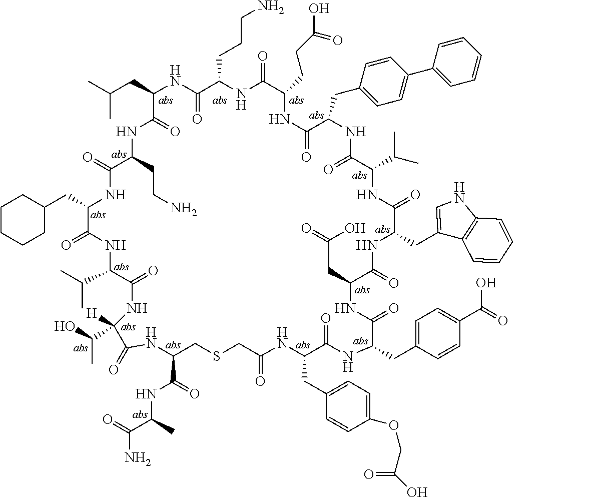 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1427 was prepared on a 50 μmol scale. The yield of the product was 20.2 mg, and its estimated purity by LCMS analysis was 96.2%. Analysis condition B: Retention time=1.64 min; ESI-MS(+) m/z [M+2H]²⁺: 1039.3.

### Preparation of Compound 1428

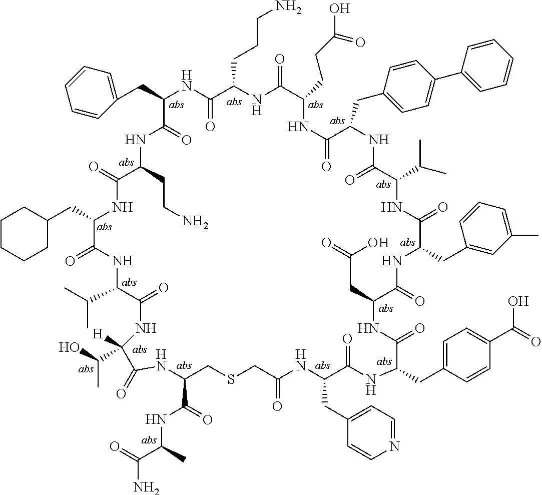 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](Cc3ccccc3)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1428 was prepared on a 50 μmol scale. The yield of the product was 10.6 mg, and its estimated purity by LCMS analysis was 94.9%. Analysis condition B: Retention time=1.54 min; ESI-MS(+) m/z [M+2H]²⁺: 1007.1.

### Preparation of Compound 1429

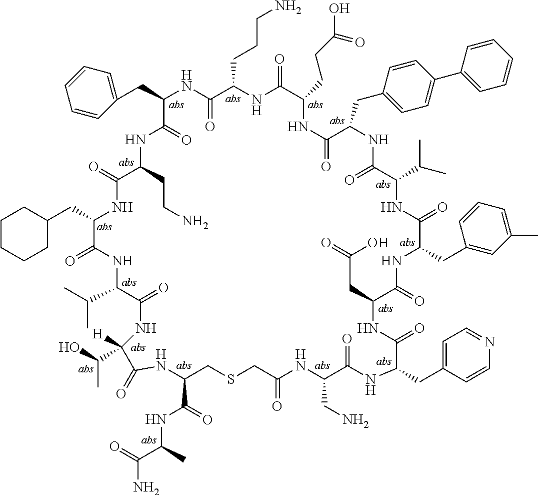 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](Cc3ccccc3)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1429 was prepared on a 50 μmol scale. The yield of the product was 15.1 mg, and its estimated purity by LCMS analysis was 100%. Analysis condition A: Retention time=1.61 min; ESI-MS(+) m/z [M+H]⁺: 1908.1.

### Preparation of Compound 1430

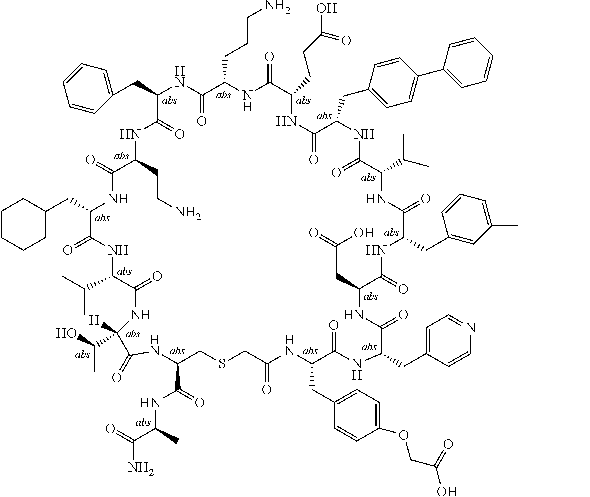 [SMILES: Cc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](Cc3ccccc3)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1430 was prepared on a 50 μmol scale. The yield of the product was 12.5 mg, and its estimated purity by LCMS analysis was 92.4%. Analysis condition A: Retention time=1.42 min; ESI-MS(+) m/z [M+3H]³⁺: 682.2.

### Preparation of Compound 1431

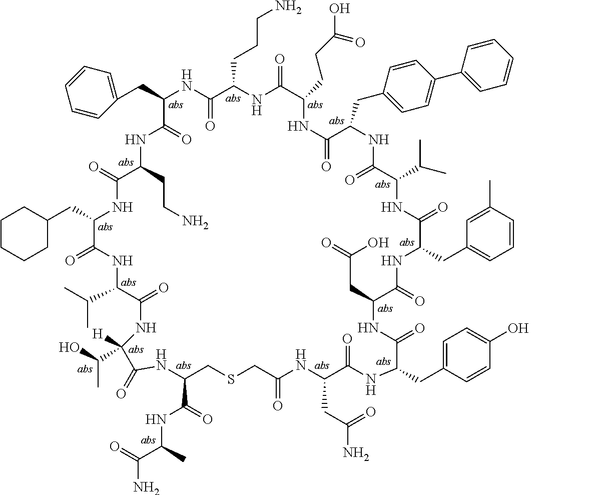 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](Cc3ccccc3)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1431 was prepared on a 50 μmol scale. The yield of the product was 13 mg, and its estimated purity by LCMS analysis was 97.3%. Analysis condition A: Retention time=1.59 min; ESI-MS(+) m/z [M+H]⁺: 1951.2.

### Preparation of Compound 1432

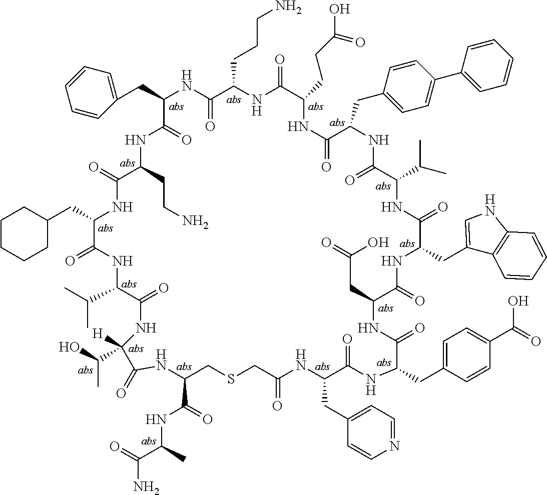 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1432 was prepared on a 50 μmol scale. The yield of the product was 13.5 mg, and its estimated purity by LCMS analysis was 95.8%. Analysis condition B: Retention time=1.43 min; ESI-MS(+) m/z [M+2H]²⁺: 1020.2.

### Preparation of Compound 1433

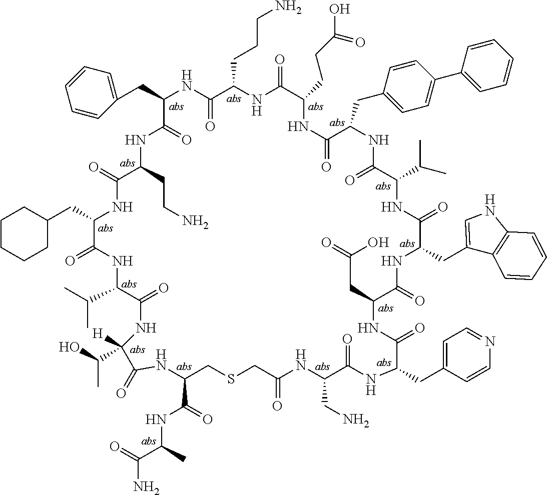 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1433 was prepared on a 50 μmol scale. The yield of the product was 16.1 mg, and its estimated purity by LCMS analysis was 98.7%. Analysis condition A: Retention time=1.51 min; ESI-MS(+) m/z [M+H]⁺: 1932.9.

### Preparation of Compound 1434

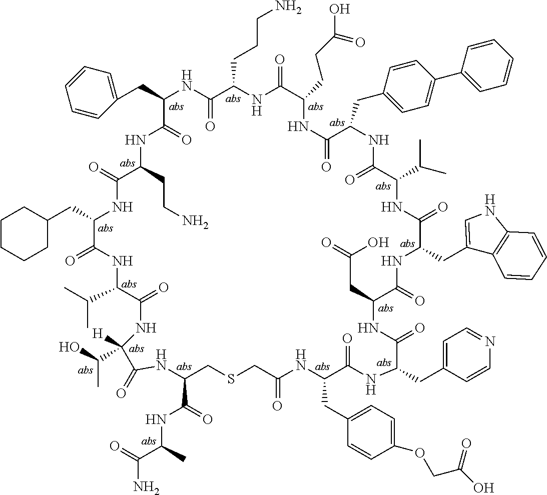 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1434 was prepared on a 50 μmol scale. The yield of the product was 24.6 mg, and its estimated purity by LCMS analysis was 100%. Analysis condition A: Retention time=1.5 min; ESI-MS(+) m/z [M+2H]²⁺: 1035.2.

### Preparation of Compound 1435

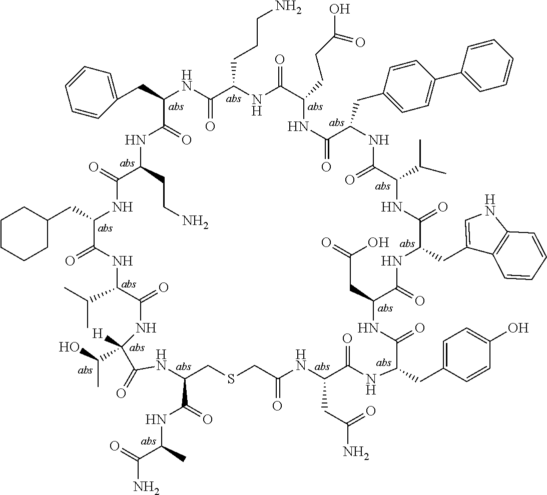 [SMILES: CC(C)[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1435 was prepared on a 50 μmol scale. The yield of the product was 14.4 mg, and its estimated purity by LCMS analysis was 93.6%. Analysis condition B: Retention time=1.52 min; ESI-MS(+) m/z [M+H]⁺: 1976.

### Preparation of Compound 1436

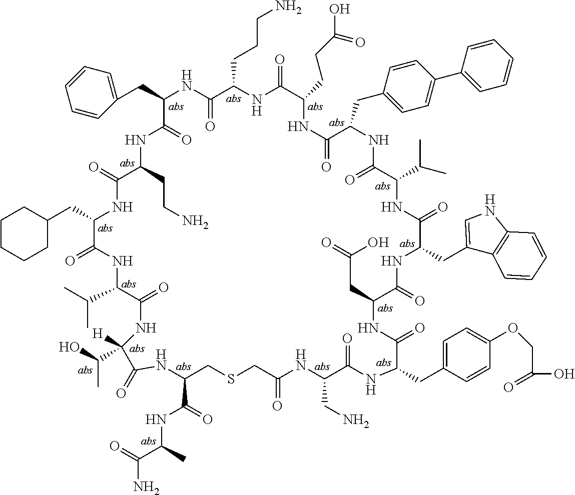 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1436 was prepared on a 50 μmol scale. The yield of the product was 13.3 mg, and its estimated purity by LCMS analysis was 94.1%. Analysis condition B: Retention time=1.44 min; ESI-MS(+) m/z [M+2H]²⁺: 1004.1.

### Preparation of Compound 1437

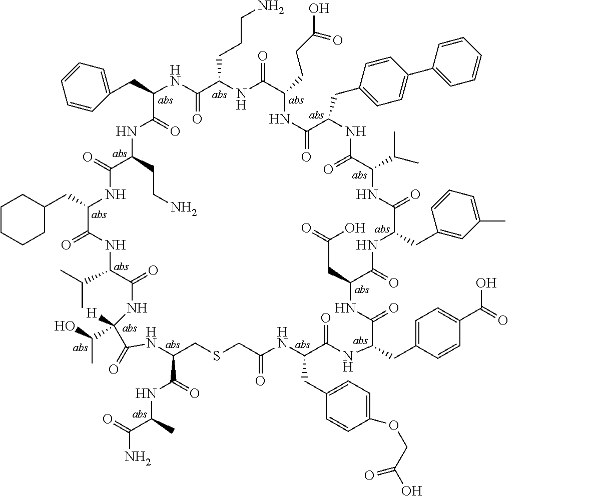 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](Cc3ccccc3)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1437 was prepared on a 50 μmol scale. The yield of the product was 17.1 mg, and its estimated purity by LCMS analysis was 94.6%. Analysis condition B: Retention time=1.7 min; ESI-MS(+) m/z [M+2H]²⁺: 1043.9.

### Preparation of Compound 1438

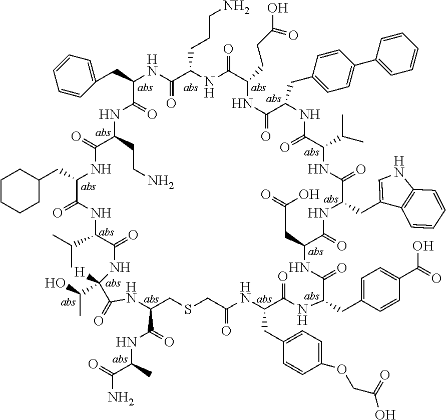 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccccc2)NC(=O)[C@H](CCCN)NC(=O)[C@H](CCC(=O)O)NC(=O)[C@H](Cc2ccc(-c3ccccc3)cc2)NC1=O]

Compound 1438 was prepared on a 50 μmol scale. The yield of the product was 16.1 mg, and its estimated purity by LCMS analysis was 96.7%. Analysis condition B: Retention time=1.56 min; ESI-MS(+) m/z [M+2H]²⁺: 1056.1.

### Preparation of Compound 1439

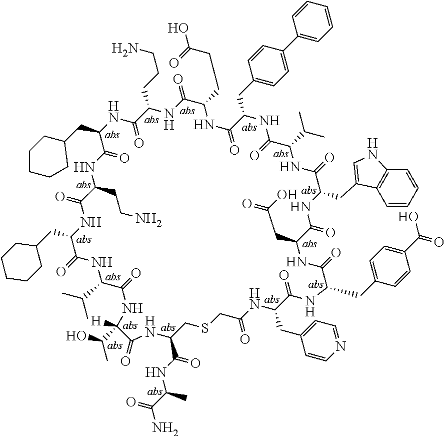 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1439 was prepared on a 50 μmol scale. The yield of the product was 22.2 mg, and its estimated purity by LCMS analysis was 91.3%. Analysis condition B: Retention time=1.62 min; ESI-MS(+) m/z [M+2H]²⁺: 1023.2.

### Preparation of Compound 1440

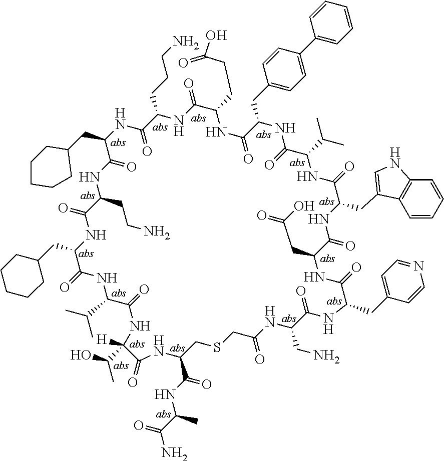 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1440 was prepared on a 50 μmol scale. The yield of the product was 18.4 mg, and its estimated purity by LCMS analysis was 91.2%. Analysis condition B: Retention time=1.54 min; ESI-MS(+) m/z [M+H]⁺: 1939.

### Preparation of Compound 1441

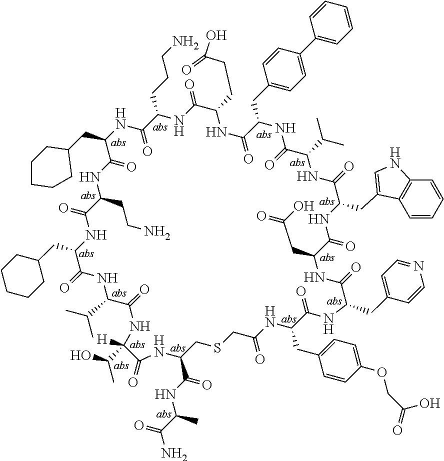 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1441 was prepared on a 50 μmol scale. The yield of the product was 14.6 mg, and its estimated purity by LCMS analysis was 95.6%. Analysis condition B: Retention time=1.54 min; ESI-MS(+) m/z [M+2H]²⁺: 1038.

### Preparation of Compound 1442

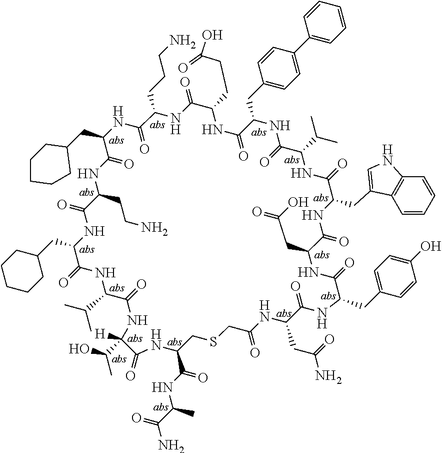 [SMILES: CC(C)[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1442 was prepared on a 50 μmol scale. The yield of the product was 21.3 mg, and its estimated purity by LCMS analysis was 98.9%. Analysis condition A: Retention time=1.74 min; ESI-MS(+) m/z [M+H]⁺: 1981.9.

### Preparation of Compound 1443

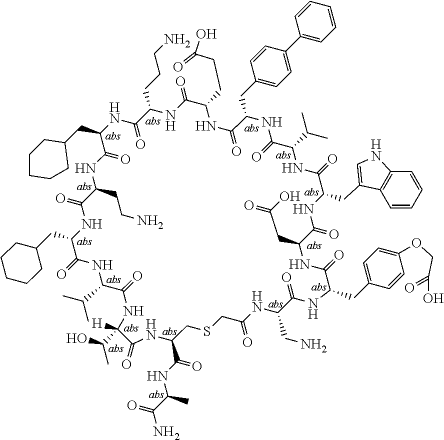 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1443 was prepared on a 50 μmol scale. The yield of the product was 13.9 mg, and its estimated purity by LCMS analysis was 90.6%. Analysis condition B: Retention time=1.55 min; ESI-MS(+) m/z [M+2H]²⁺: 1006.3.

### Preparation of Compound 1444

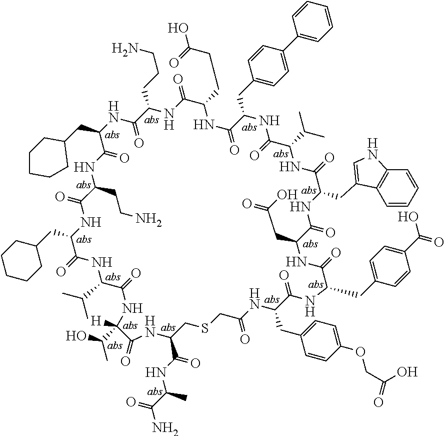 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCC(=O)O)NC(=O)[C@H](Cc2ccc(-c3ccccc3)cc2)NC1=O]

Compound 1444 was prepared on a 50 μmol scale. The yield of the product was 22.6 mg, and its estimated purity by LCMS analysis was 84.9%. Analysis condition A: Retention time=1.53 min; ESI-MS(+) m/z [M+2H]²⁺: 1059.9.

### Preparation of Compound 1445

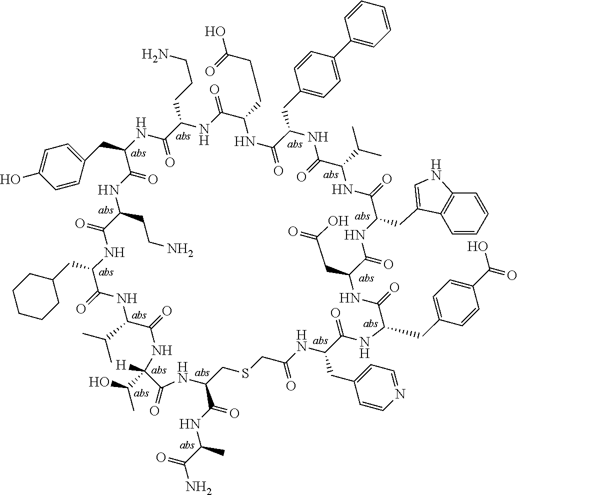 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccc(O)cc2)NC(=O)[C@H](CCCN)NC(=O)[C@H](CCC(=O)O)NC(=O)[C@H](Cc2ccc(-c3ccccc3)cc2)NC1=O]

Compound 1445 was prepared on a 50 μmol scale. The yield of the product was 20.3 mg, and its estimated purity by LCMS analysis was 95.7%. Analysis condition B: Retention time=1.35 min; ESI-MS(+) m/z [M+3H]³⁺: 685.9.

### Preparation of Compound 1446

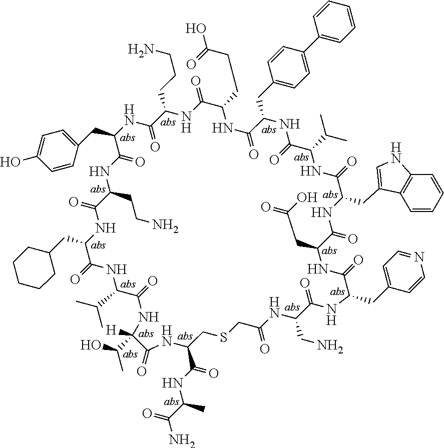 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccc(O)cc2)NC(=O)[C@H](CCCN)NC(=O)[C@H](CCC(=O)O)NC(=O)[C@H](Cc2ccc(-c3ccccc3)cc2)NC1=O]

Compound 1446 was prepared on a 50 μmol scale. The yield of the product was 22.1 mg, and its estimated purity by LCMS analysis was 87.1%. Analysis condition A: Retention time=1.41 min; ESI-MS(+) m/z [M+H]⁺: 1949.5.

### Preparation of Compound 1447

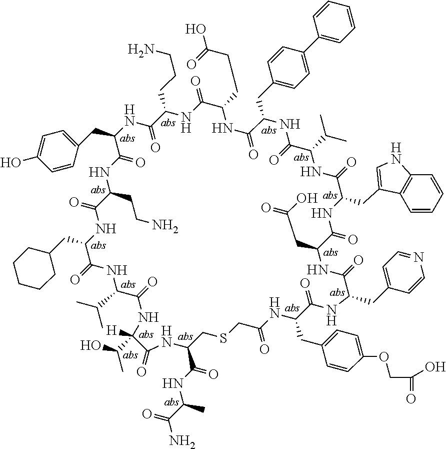 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccc(O)cc2)NC(=O)[C@H](CCCN)NC(=O)[C@H](CCC(=O)O)NC(=O)[C@H](Cc2ccc(-c3ccccc3)cc2)NC1=O]

Compound 1447 was prepared on a 50 μmol scale. The yield of the product was 23.1 mg, and its estimated purity by LCMS analysis was 90.5%. Analysis condition B: Retention time=1.42 min; ESI-MS(+) m/z [M+2H]²⁺: 1043.1.

### Preparation of Compound 1448

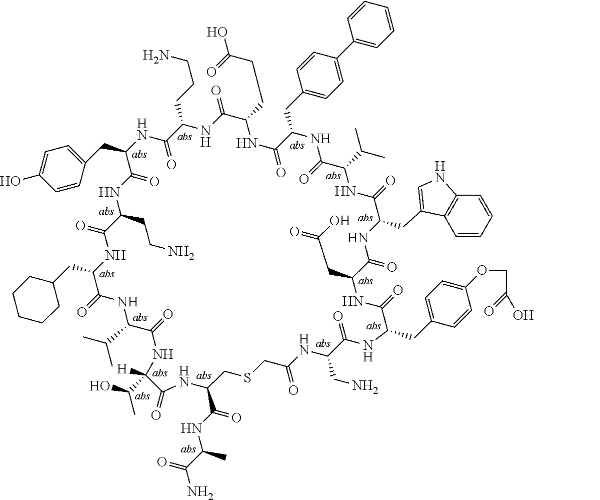 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccc(O)cc2)NC(=O)[C@H](CCCN)NC(=O)[C@H](CCC(=O)O)NC(=O)[C@H](Cc2ccc(-c3ccccc3)cc2)NC1=O]

Compound 1448 was prepared on a 50 μmol scale. The yield of the product was 21.5 mg, and its estimated purity by LCMS analysis was 95.2%. Analysis condition B: Retention time=1.33 min; ESI-MS(+) m/z [M+3H]³⁺: 675.

### Preparation of Compound 1449

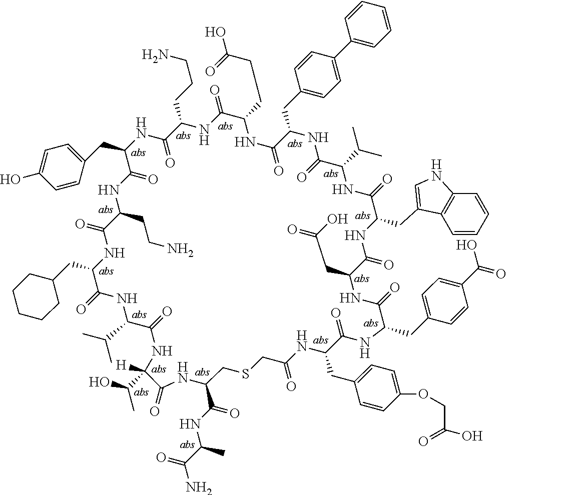 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](Cc2ccc(O)cc2)NC(=O)[C@H](CCCN)NC(=O)[C@H](CCC(=O)O)NC(=O)[C@H](Cc2ccc(-c3ccccc3)cc2)NC1=O]

Compound 1449 was prepared on a 50 μmol scale. The yield of the product was 21.1 mg, and its estimated purity by LCMS analysis was 90.6%. Analysis condition A: Retention time=1.32 min; ESI-MS(+) m/z [M+2H]²⁺: 1064.5.

### Preparation of Compound 1450

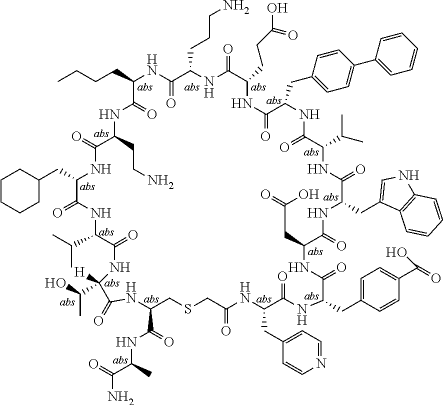 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1450 was prepared on a 50 μmol scale. The yield of the product was 16.1 mg, and its estimated purity by LCMS analysis was 85.5%. Analysis condition A: Retention time=1.49 min; ESI-MS(+) m/z [M+2H]²⁺: 1003.

### Preparation of Compound 1451

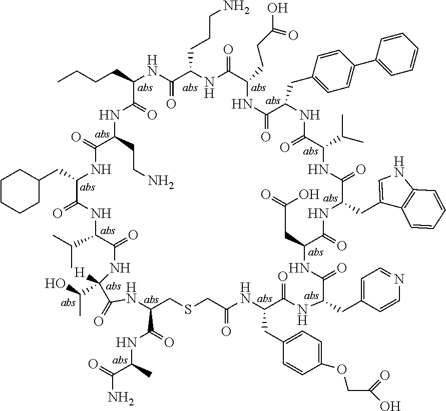 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1451 was prepared on a 50 μmol scale. The yield of the product was 14.4 mg, and its estimated purity by LCMS analysis was 90.3%. Analysis condition B: Retention time=1.51 min; ESI-MS(+) m/z [M+2H]²⁺: 1018.

### Preparation of Compound 1452

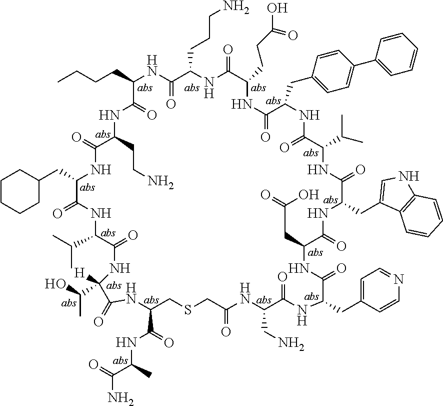 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1452 was prepared on a 50 μmol scale. The yield of the product was 12.1 mg, and its estimated purity by LCMS analysis was 90.6%. Analysis condition B: Retention time=1.35 min; ESI-MS(+) m/z [M+H]⁺: 1899.3.

### Preparation of Compound 1453

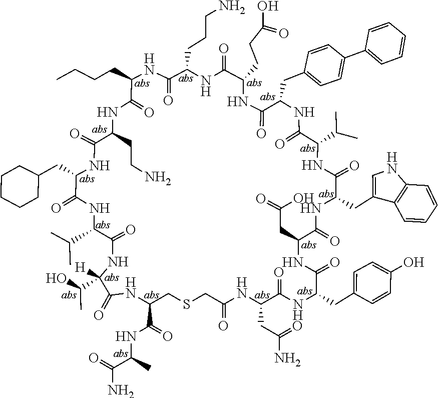 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1453 was prepared on a 50 μmol scale. The yield of the product was 21.3 mg, and its estimated purity by LCMS analysis was 92%. Analysis condition B: Retention time=1.5 min; ESI-MS(+) m/z [M+2H]²⁺: 972.1.

### Preparation of Compound 1454

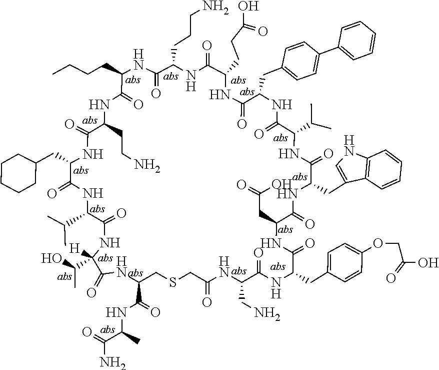 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1454 was prepared on a 50 μmol scale. The yield of the product was 12.3 mg, and its estimated purity by LCMS analysis was 87.6%. Analysis condition B: Retention time=1.52 min; ESI-MS(+) m/z [M+H]⁺: 1971.7.

### Preparation of Compound 1455

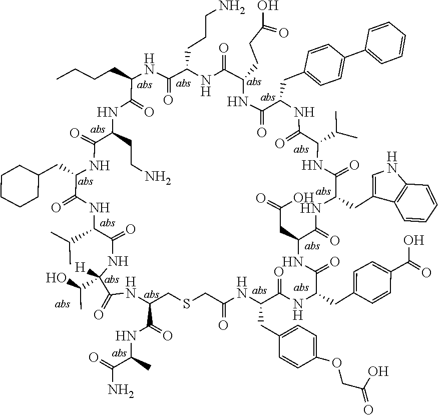 [SMILES: CCCC[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1455 was prepared on a 50 μmol scale. The yield of the product was 12.5 mg, and its estimated purity by LCMS analysis was 97.6%. Analysis condition A: Retention time=1.48 min; ESI-MS(+) m/z [M+2H]²⁺: 1039.2.

### Preparation of Compound 1456

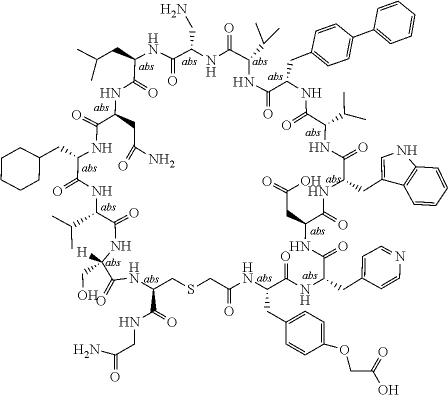 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC1=O]

Compound 1456 was prepared on a 50 μmol scale. The yield of the product was 19.4 mg, and its estimated purity by LCMS analysis was 96%. Analysis condition B: Retention time=1.57 min; ESI-MS(+) m/z [M+H]⁺: 1962.1.

### Preparation of Compound 1457

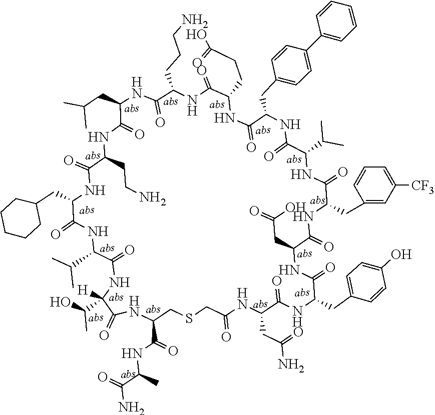 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1457 was prepared on a 50 μmol scale. The yield of the product was 10.9 mg, and its estimated purity by LCMS analysis was 90.4%. Analysis condition A: Retention time=1.69 min; ESI-MS(+) m/z [M+H]⁺: 1972.2.

### Preparation of Compound 1458

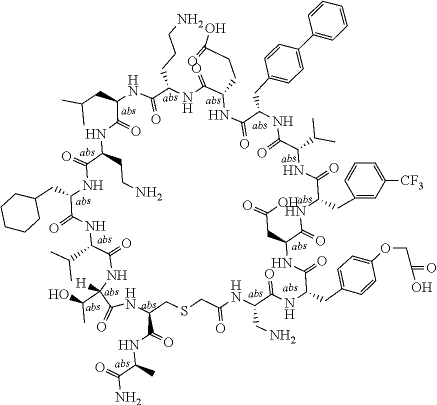 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1458 was prepared on a 50 μmol scale. The yield of the product was 8.1 mg, and its estimated purity by LCMS analysis was 89.1%. Analysis condition A: Retention time=1.61, 1.66 min; ESI-MS(+) m/z [M+3H]³⁺: 667.82, 668.06.

### Preparation of Compound 1459

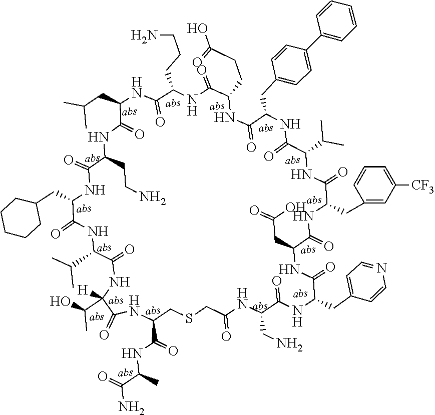 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1459 was prepared on a 50 μmol scale. The yield of the product was 7.6 mg, and its estimated purity by LCMS analysis was 92.7%. Analysis condition B: Retention time=1.58 min; ESI-MS(+) m/z [M+H]⁺: 1929.1.

### Preparation of Compound 1460

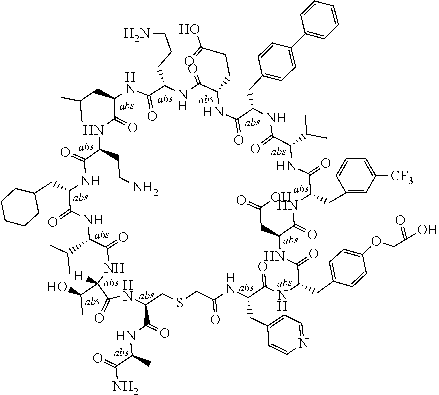 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1460 was prepared on a 50 μmol scale. The yield of the product was 4.2 mg, and its estimated purity by LCMS analysis was 94.1%. Analysis condition A: Retention time=1.6 min; ESI-MS(+) m/z [M+2H]²⁺: 1032.3.

### Preparation of Compound 1461

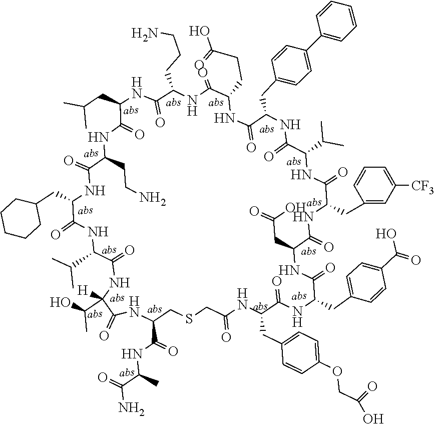 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1461 was prepared on a 50 μmol scale. The yield of the product was 12.2 mg, and its estimated purity by LCMS analysis was 100%. Analysis condition A: Retention time=1.52 min; ESI-MS(+) m/z [M+2H]²⁺: 1054.1.

### Preparation of Compound 1462

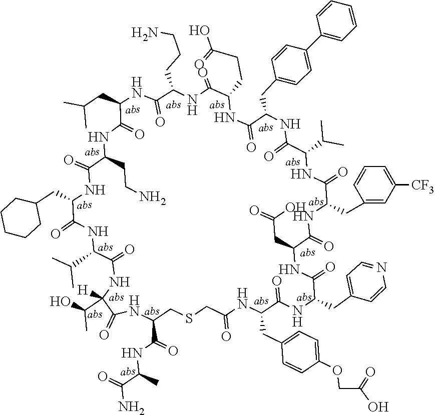 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1462 was prepared on a 50 μmol scale. The yield of the product was 15.2 mg, and its estimated purity by LCMS analysis was 90.4%. Analysis condition A: Retention time=1.58 min; ESI-MS(+) m/z [M+3H]³⁺: 688.6.

### Preparation of Compound 1463

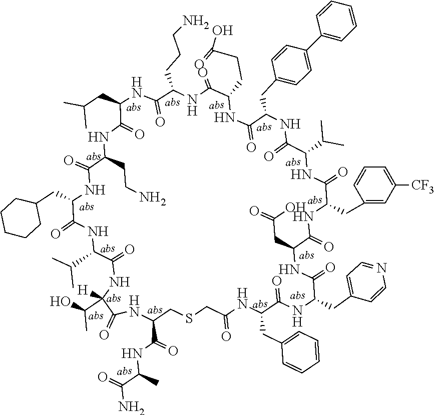 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccncc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1463 was prepared on a 50 μmol scale. The yield of the product was 8.3 mg, and its estimated purity by LCMS analysis was 97.9%. Analysis condition A: Retention time=1.74 min; ESI-MS(+) m/z [M+3H]³⁺: 664.2.

### Preparation of Compound 1464

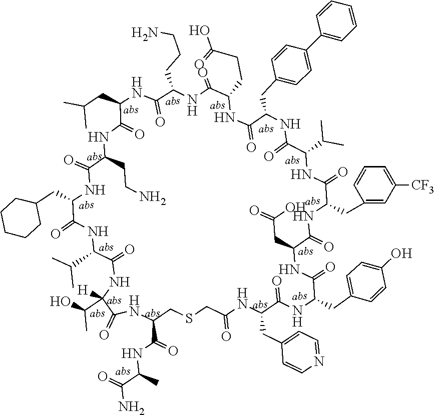 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1464 was prepared on a 50 μmol scale. The yield of the product was 10.5 mg, and its estimated purity by LCMS analysis was 86.7%. Analysis condition A: Retention time=1.66 min; ESI-MS(+) m/z [M+2H]²⁺: 1003.1.

### Preparation of Compound 1465

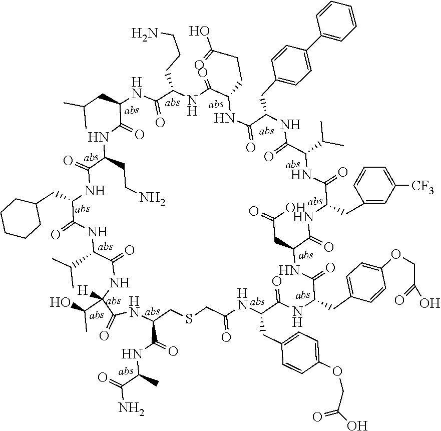 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1465 was prepared on a 50 μmol scale. The yield of the product was 15.3 mg, and its estimated purity by LCMS analysis was 90%. Analysis condition A: Retention time=1.47, 1.53 min; ESI-MS(+) m/z [M+2H]²⁺: 1068.9, 1068.45.

### Preparation of Compound 1466

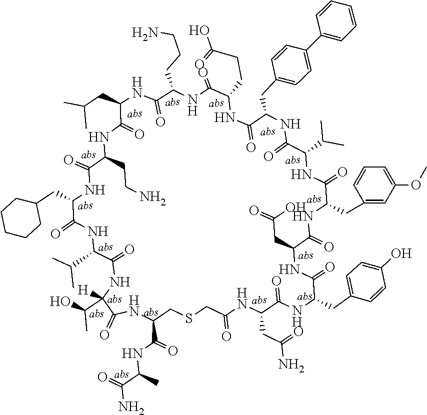 [SMILES: COc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1466 was prepared on a 50 μmol scale. The yield of the product was 4.9 mg, and its estimated purity by LCMS analysis was 92.8%. Analysis condition A: Retention time=1.58 min; ESI-MS(+) m/z [M+H]⁺: 1934.2.

### Preparation of Compound 1467

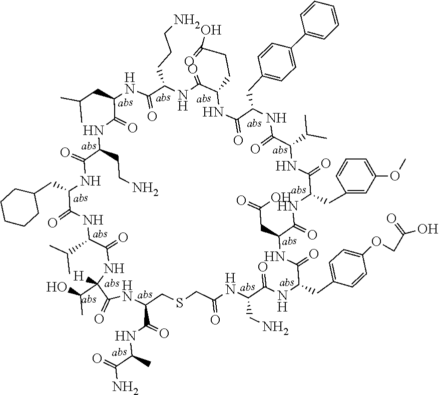 [SMILES: COc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1467 was prepared on a 50 μmol scale. The yield of the product was 1.6 mg, and its estimated purity by LCMS analysis was 96.3%. Analysis condition B: Retention time=1.5 min; ESI-MS(+) m/z [M+H]⁺: 1964.1.

### Preparation of Compound 1468

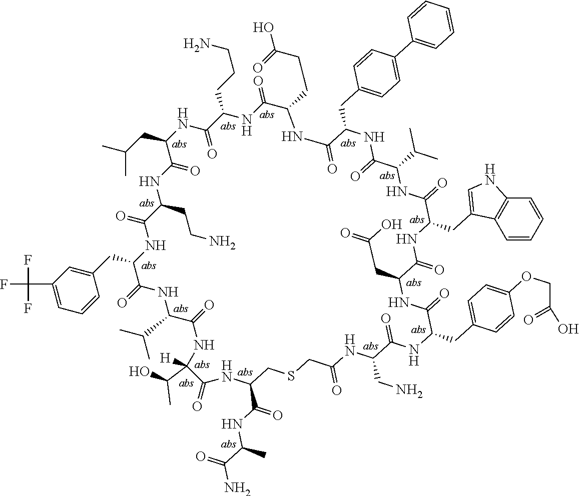 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(C(F)(F)F)c2)NC(=O)[C@H](CCN)NC1=O]

Compound 1468 was prepared on a 50 μmol scale. The yield of the product was 10.3 mg, and its estimated purity by LCMS analysis was 97.5%. Analysis condition B: Retention time=1.43 min; ESI-MS(+) m/z [M+2H]²⁺: 1018.

### Preparation of Compound 1469

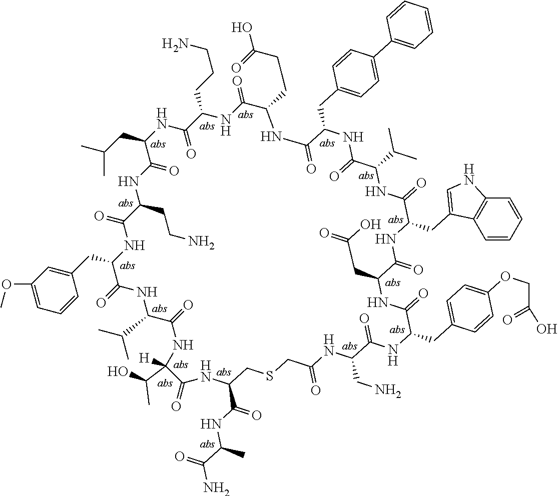 [SMILES: COc1cccc(C[C@@H]2NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC2=O)c1]

Compound 1469 was prepared on a 50 μmol scale. The yield of the product was 9 mg, and its estimated purity by LCMS analysis was 93.8%. Analysis condition B: Retention time=1.33 min; ESI-MS(+) m/z [M+2H]²⁺: 999.

### Preparation of Compound 1470

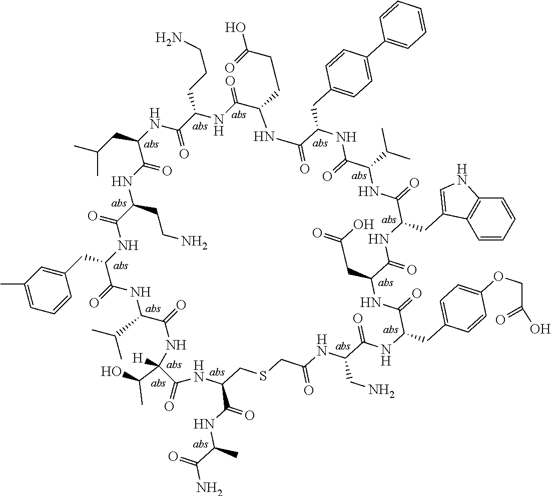 [SMILES: Cc1cccc(C[C@@H]2NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC2=O)c1]

Compound 1470 was prepared on a 50 μmol scale. The yield of the product was 17.7 mg, and its estimated purity by LCMS analysis was 81.1%. Analysis condition A: Retention time=1.44 min; ESI-MS(+) m/z [M+2H]²⁺: 991.1.

### Preparation of Compound 1471

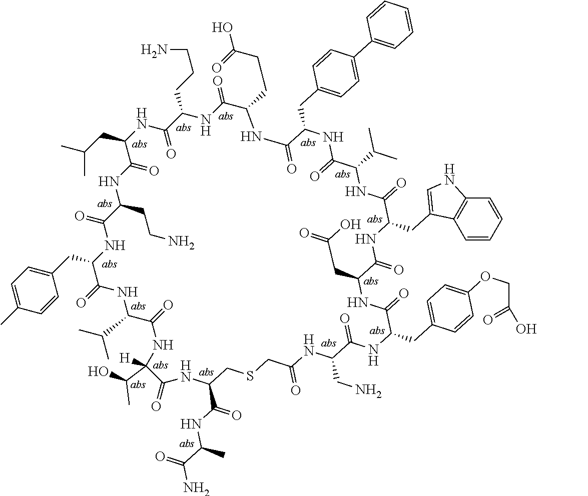 [SMILES: Cc1ccc(C[C@@H]2NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC2=O)cc1]

Compound 1471 was prepared on a 50 μmol scale. The yield of the product was 10.8 mg, and its estimated purity by LCMS analysis was 84.8%. Analysis condition B: Retention time=1.56 min; ESI-MS(+) m/z [M+2H]²⁺: 991.

### Preparation of Compound 1472

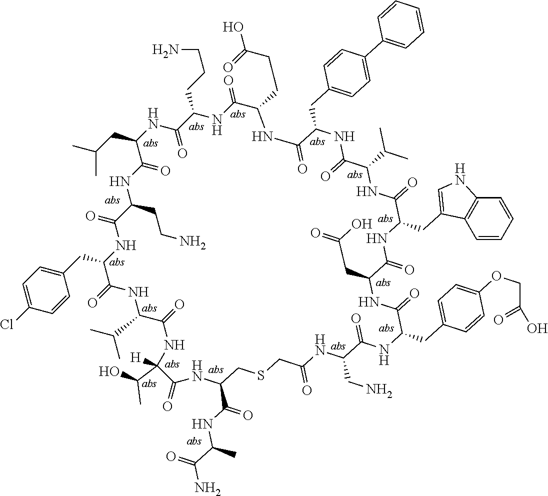 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(Cl)cc2)NC(=O)[C@H](CCN)NC1=O]

Compound 1472 was prepared on a 50 μmol scale. The yield of the product was 6.4 mg, and its estimated purity by LCMS analysis was 98.9%. Analysis condition A: Retention time=1.48, 1.54 min; ESI-MS(+) m/z [M+2H]²⁺: 1000.19, 1001.09.

### Preparation of Compound 1473

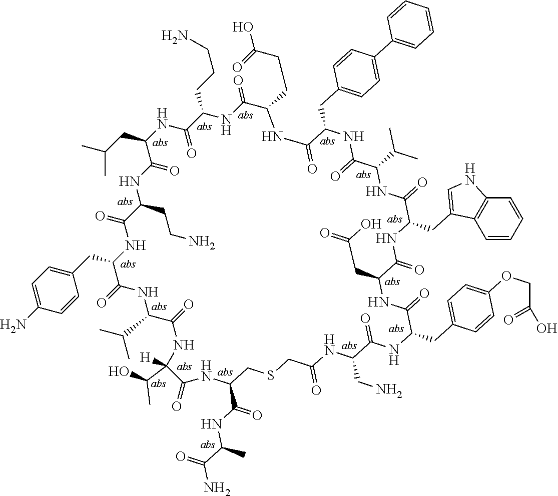 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(N)cc2)NC(=O)[C@H](CCN)NC1=O]

Compound 1473 was prepared on a 50 μmol scale. The yield of the product was 6.1 mg, and its estimated purity by LCMS analysis was 97.9%. Analysis condition A: Retention time=1.38 min; ESI-MS(+) m/z [M+H]⁺: 1982.

### Preparation of Compound 1474

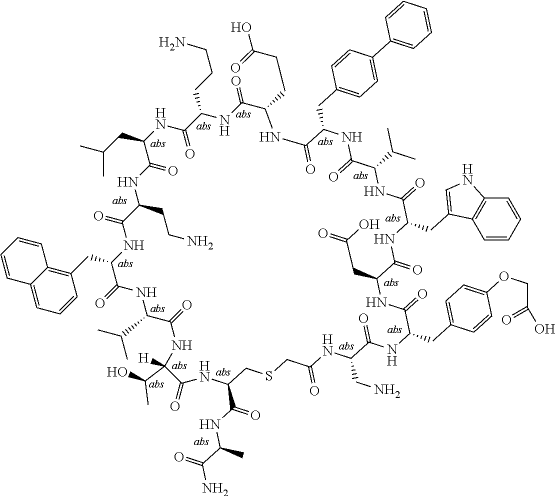 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc3ccccc23)NC(=O)[C@H](CCN)NC1=O]

Compound 1474 was prepared on a 50 μmol scale. The yield of the product was 10.4 mg, and its estimated purity by LCMS analysis was 85.6%. Analysis condition A: Retention time=1.48 min; ESI-MS(+) m/z [M+2H]²⁺: 1009.2.

### Preparation of Compound 1475

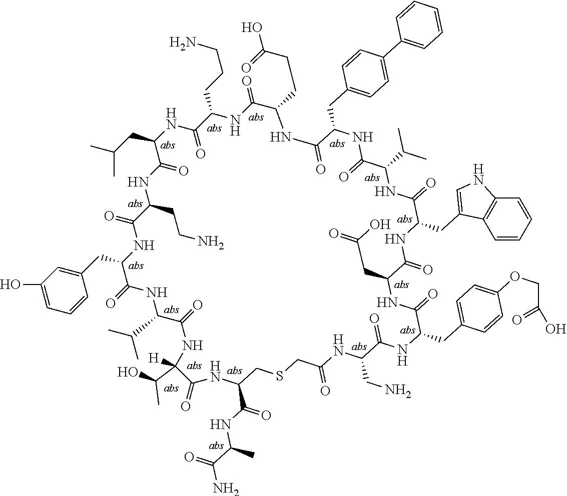 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(O)c2)NC(=O)[C@H](CCN)NC1=O]

Compound 1475 was prepared on a 50 μmol scale. The yield of the product was 6.3 mg, and its estimated purity by LCMS analysis was 83.7%. Analysis condition B: Retention time=1.45 min; ESI-MS(+) m/z [M+2H]²⁺: 992.

### Preparation of Compound 1476

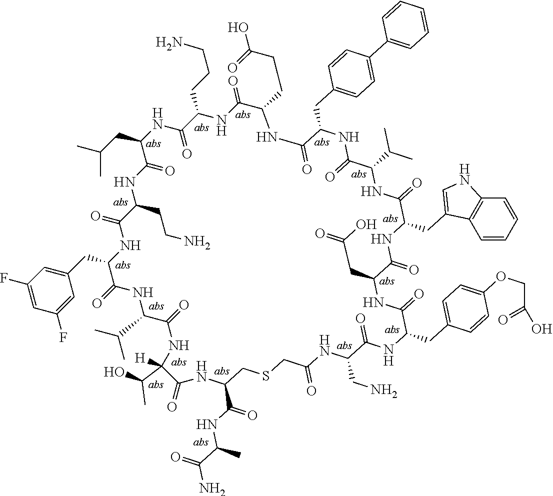 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cc(F)cc(F)c2)NC(=O)[C@H](CCN)NC1=O]

Compound 1476 was prepared on a 50 μmol scale. The yield of the product was 7.7 mg, and its estimated purity by LCMS analysis was 94.4%. Analysis condition B: Retention time=1.5 min; ESI-MS(+) m/z [M+2H]²⁺: 1001.2.

### Preparation of Compound 1477

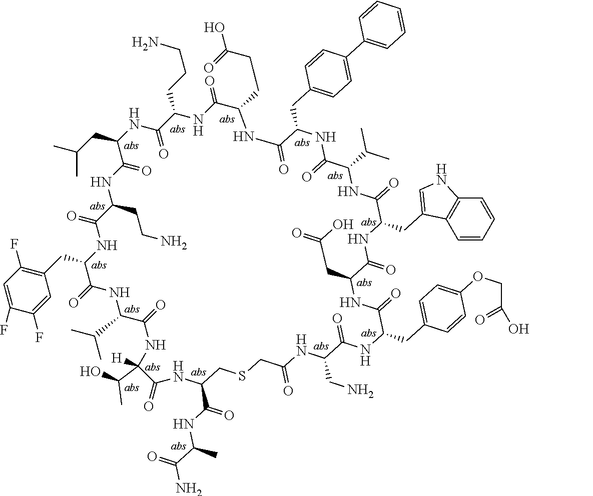 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cc(F)c(F)cc2F)NC(=O)[C@H](CCN)NC1=O]

Compound 1477 was prepared on a 50 μmol scale. The yield of the product was 12.8 mg, and its estimated purity by LCMS analysis was 100%. Analysis condition B: Retention time=1.53 min; ESI-MS(+) m/z [M+2H]²⁺: 1011.1.

### Preparation of Compound 1478

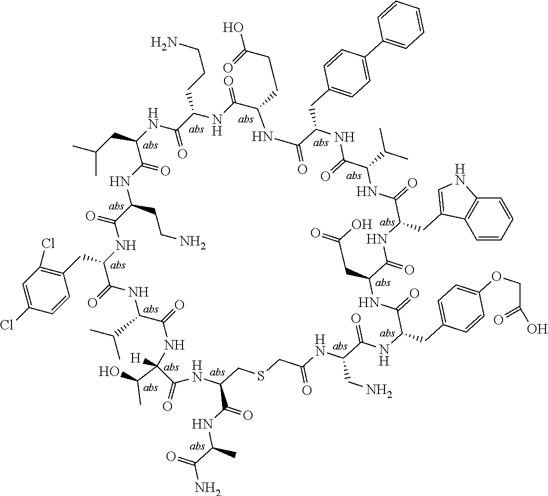 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(Cl)cc2Cl)NC(=O)[C@H](CCN)NC1=O]

Compound 1478 was prepared on a 50 μmol scale. The yield of the product was 23.4 mg, and its estimated purity by LCMS analysis was 91.3%. Analysis condition A: Retention time=1.52, 1.57, 1.58 min; ESI-MS(+) m/z [M+2H]²⁺: 1018.06, 1018.09, 1018.09.

### Preparation of Compound 1479

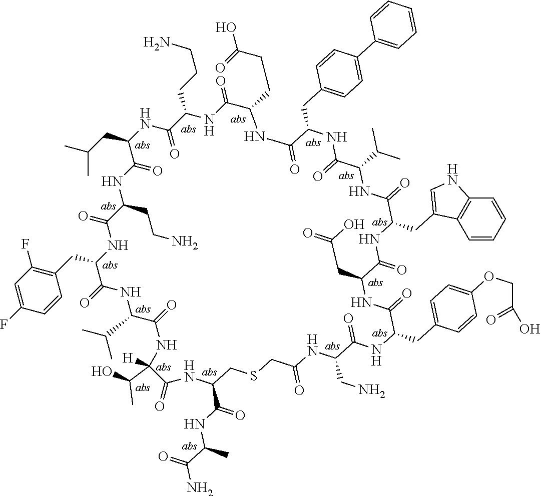 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(F)cc2F)NC(=O)[C@H](CCN)NC1=O]

Compound 1479 was prepared on a 50 μmol scale. The yield of the product was 10.2 mg, and its estimated purity by LCMS analysis was 100%. Analysis condition A: Retention time=1.48 min; ESI-MS(+) m/z [M+2H]²⁺: 1001.4.

### Preparation of Compound 1480

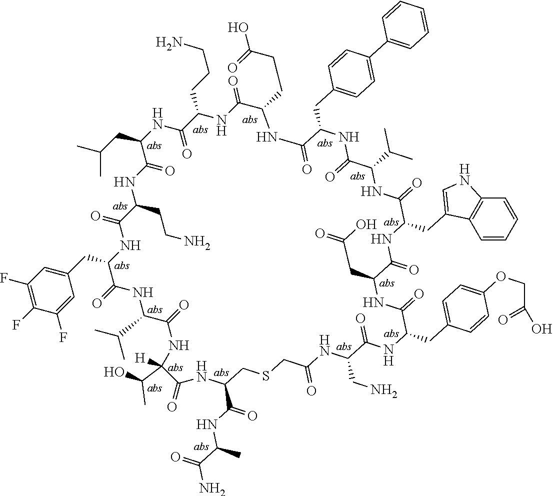 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cc(F)c(F)c(F)c2)NC(=O)[C@H](CCN)NC1=O]

Compound 1480 was prepared on a 50 μmol scale. The yield of the product was 18.2 mg, and its estimated purity by LCMS analysis was 100%. Analysis condition A: Retention time=1.51 min; ESI-MS(+) m/z [M+2H]²⁺: 1010.9.

### Preparation of Compound 1481

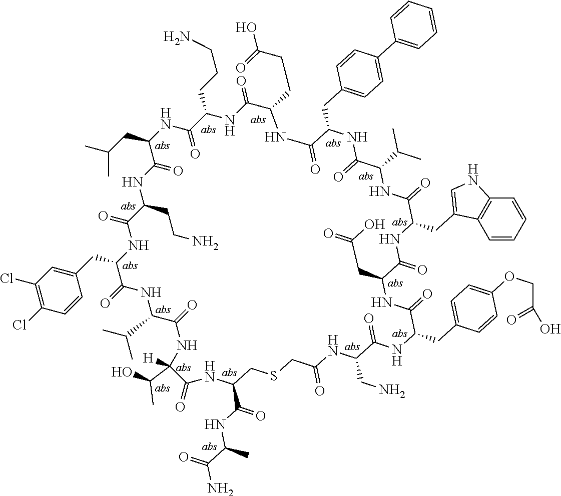 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(Cl)c(Cl)c2)NC(=O)[C@H](CCN)NC1=O]

Compound 1481 was prepared on a 50 μmol scale. The yield of the product was 5.8 mg, and its estimated purity by LCMS analysis was 100%. Analysis condition A: Retention time=1.59 min; ESI-MS(+) m/z [M+2H]²⁺: 1018.1.

### Preparation of Compound 1482

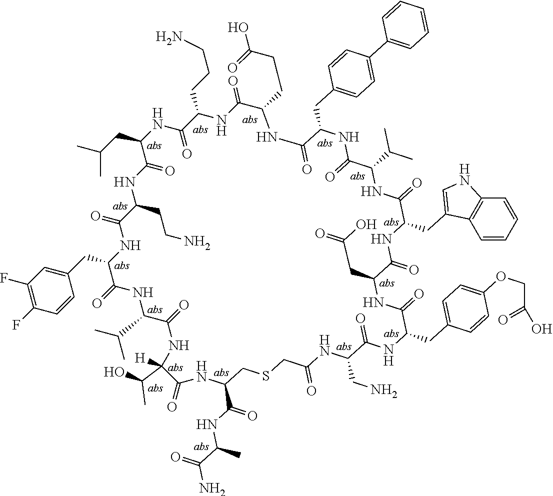 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(F)c(F)c2)NC(=O)[C@H](CCN)NC1=O]

Compound 1482 was prepared on a 50 μmol scale. The yield of the product was 9.9 mg, and its estimated purity by LCMS analysis was 90.9%. Analysis condition B: Retention time=1.53 min; ESI-MS(+) m/z [M+2H]²⁺: 1001.6.

### Preparation of Compound 1483

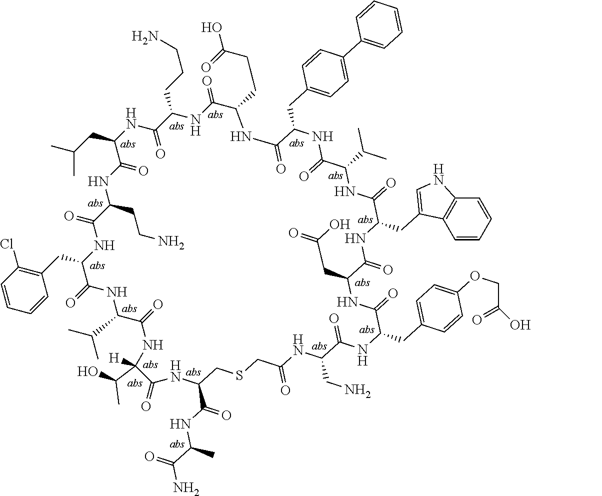 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2Cl)NC(=O)[C@H](CCN)NC1=O]

Compound 1483 was prepared on a 50 μmol scale. The yield of the product was 11.6 mg, and its estimated purity by LCMS analysis was 95.8%. Analysis condition B: Retention time=1.46 min; ESI-MS(+) m/z [M+2H]²⁺: 1000.9.

### Preparation of Compound 1484

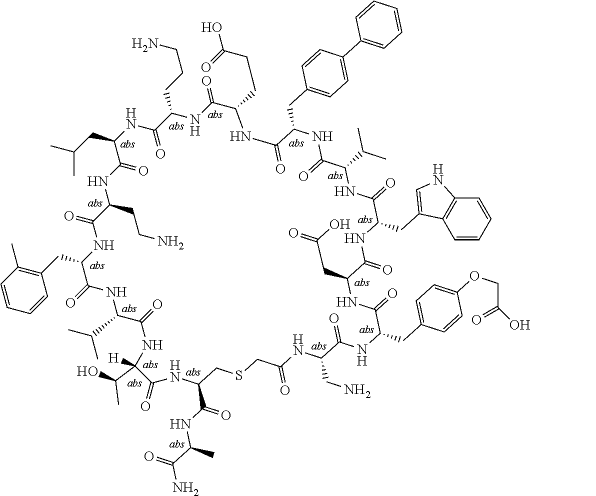 [SMILES: Cc1ccccc1C[C@@H]1NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC1=O]

Compound 1484 was prepared on a 50 μmol scale. The yield of the product was 14.8 mg, and its estimated purity by LCMS analysis was 93.5%. Analysis condition B: Retention time=1.31 min; ESI-MS(+) m/z [M+3H]³⁺: 661.2.

### Preparation of Compound 1485

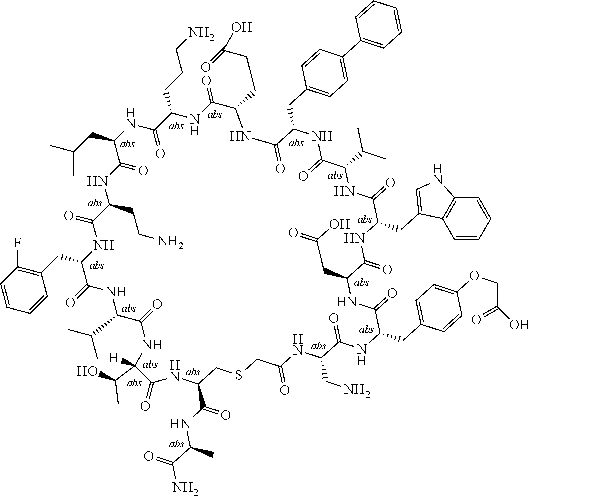 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2F)NC(=O)[C@H](CCN)NC1=O]

Compound 1485 was prepared on a 50 μmol scale. The yield of the product was 13.4 mg, and its estimated purity by LCMS analysis was 99.2%. Analysis condition A: Retention time=1.39 min; ESI-MS(+) m/z [M+2H]²⁺: 992.9.

### Preparation of Compound 1486

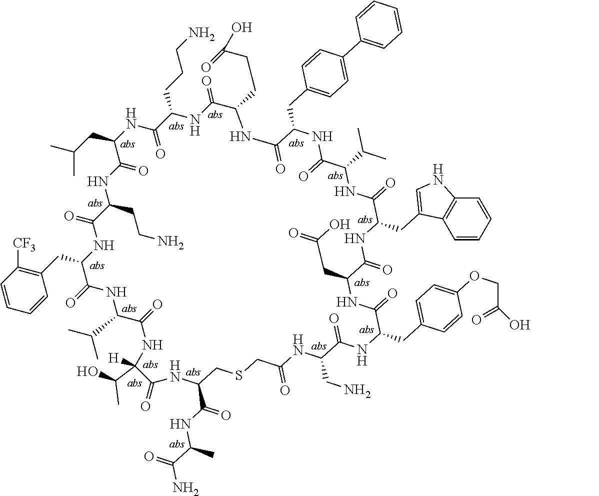 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2C(F)(F)F)NC(=O)[C@H](CCN)NC1=O]

Compound 1486 was prepared on a 50 μmol scale. The yield of the product was 10 mg, and its estimated purity by LCMS analysis was 91.6%. Analysis condition B: Retention time=1.52 min; ESI-MS(+) m/z [M+2H]²⁺: 1018.1.

### Preparation of Compound 1487

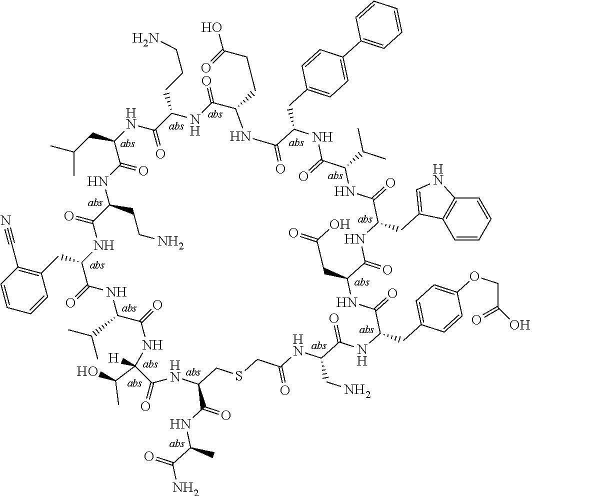 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2C#N)NC(=O)[C@H](CCN)NC1=O]

Compound 1487 was prepared on a 50 μmol scale. The yield of the product was 12.5 mg, and its estimated purity by LCMS analysis was 97.4%. Analysis condition B: Retention time=1.32 min; ESI-MS(+) m/z [M+3H]³⁺: 664.8.

### Preparation of Compound 1488

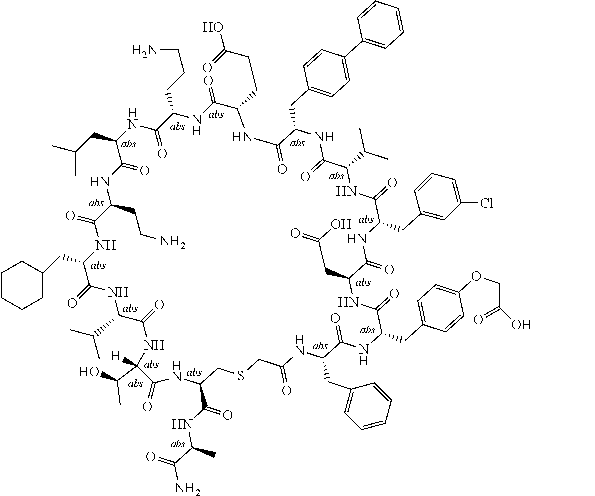 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1488 was prepared on a 50 μmol scale. The yield of the product was 8.1 mg, and its estimated purity by LCMS analysis was 99.2%. Analysis condition B: Retention time=1.8 min; ESI-MS(+) m/z [M+2H]²⁺: 1015.1.

### Preparation of Compound 1489

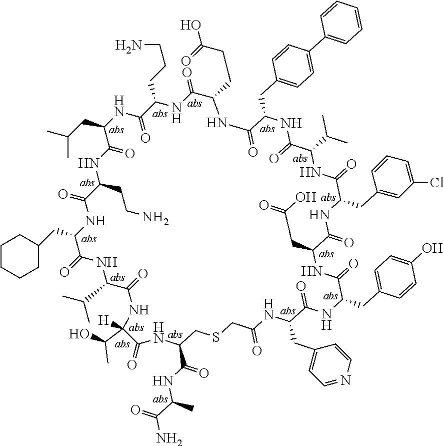 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1489 was prepared on a 50 μmol scale. The yield of the product was 6.8 mg, and its estimated purity by LCMS analysis was 100%. Analysis condition B: Retention time=1.6 min; ESI-MS(+) m/z [M+H]⁺: 1970.7.

### Preparation of Compound 1490

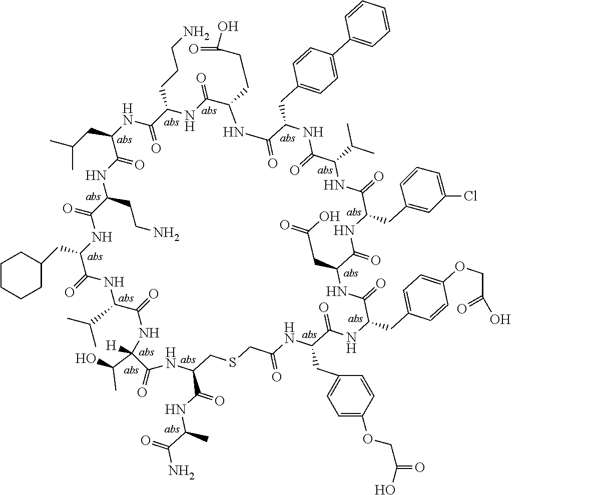 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1490 was prepared on a 50 μmol scale. The yield of the product was 17.6 mg, and its estimated purity by LCMS analysis was 100%. Analysis condition A: Retention time=1.52 min; ESI-MS(+) m/z [M+2H]²⁺: 1052.2.

### Preparation of Compound 1491

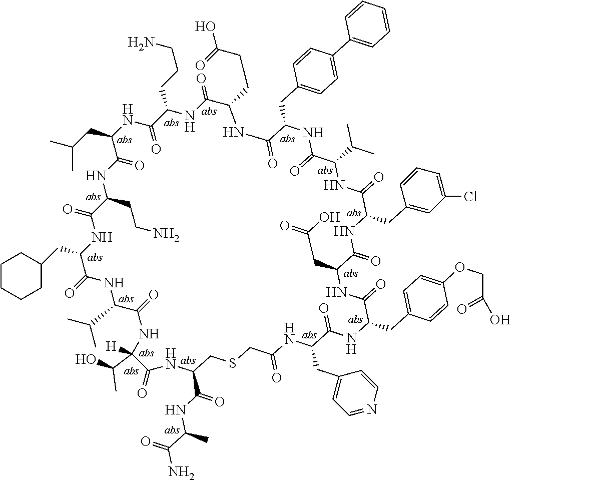 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1491 was prepared on a 50 μmol scale. The yield of the product was 6.5 mg, and its estimated purity by LCMS analysis was 94.8%. Analysis condition B: Retention time=1.61, 1.66 min; ESI-MS(+) m/z [M+3H]³⁺, [M+2H]²⁺: 677.06, 1015.12.

### Preparation of Compound 1492

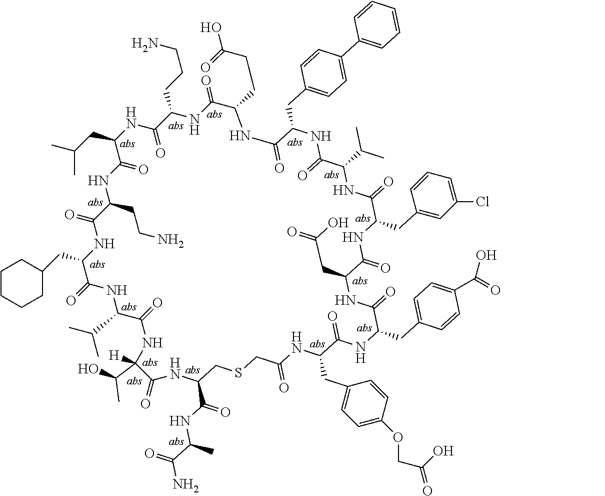 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1492 was prepared on a 50 μmol scale. The yield of the product was 12.3 mg, and its estimated purity by LCMS analysis was 92.1%. Analysis condition A: Retention time=1.45 min; ESI-MS(+) m/z [M+2H]²⁺: 1037.2.

### Preparation of Compound 1493

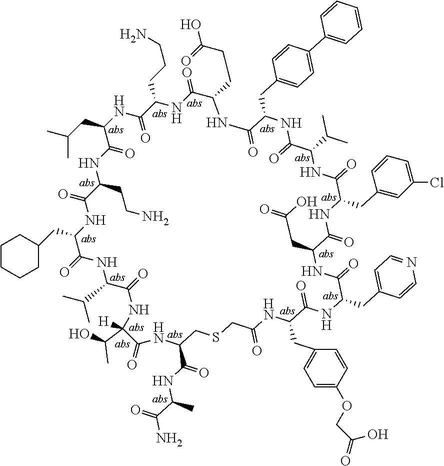 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1493 was prepared on a 50 μmol scale. The yield of the product was 7 mg, and its estimated purity by LCMS analysis was 90.4%. Analysis condition B: Retention time=1.43 min; ESI-MS(+) m/z [M+2H]²⁺: 1015.3.

### Preparation of Compound 1494

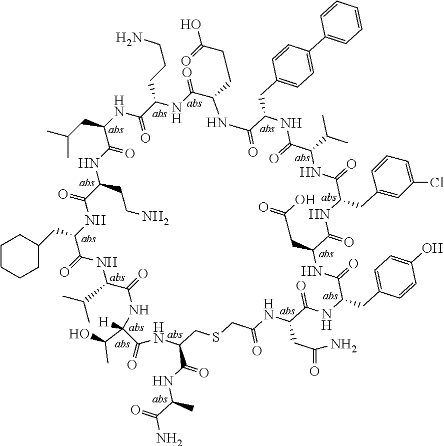 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1494 was prepared on a 50 μmol scale. The yield of the product was 6 mg, and its estimated purity by LCMS analysis was 95.3%. Analysis condition B: Retention time=1.53 min; ESI-MS(+) m/z [M+2H]²⁺: 970.

### Preparation of Compound 1495

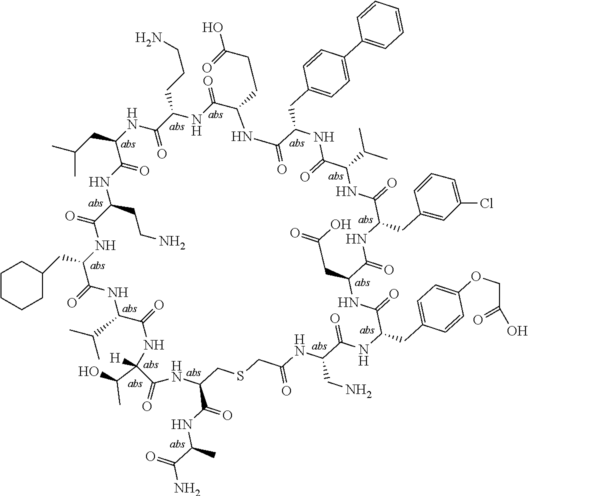 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1495 was prepared on a 50 μmol scale. The yield of the product was 6.8 mg, and its estimated purity by LCMS analysis was 100%. Analysis condition A: Retention time=1.6 min; ESI-MS(+) m/z [M+H]⁺: 1968.2.

### Preparation of Compound 1496

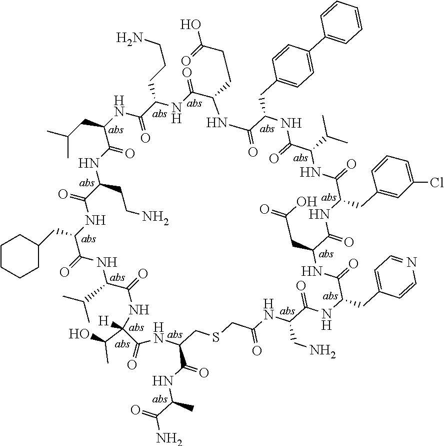 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1496 was prepared on a 50 μmol scale. The yield of the product was 12.5 mg, and its estimated purity by LCMS analysis was 90.3%. Analysis condition A: Retention time=1.55 min; ESI-MS(+) m/z [M+2H]²⁺: 948.2.

### Preparation of Compound 1497

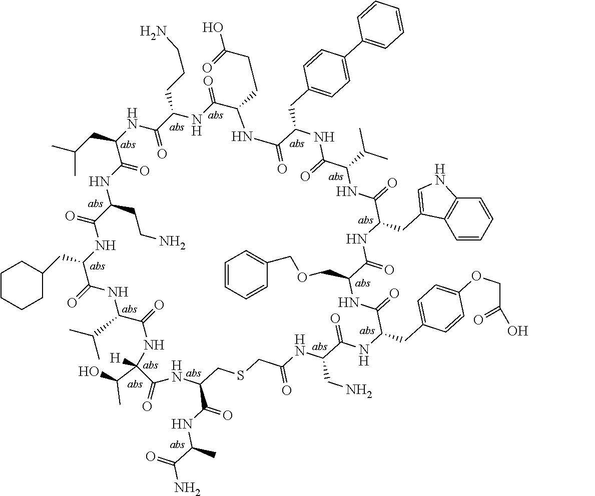 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](COCc2ccccc2)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1497 was prepared on a 50 μmol scale. The yield of the product was 4.7 mg, and its estimated purity by LCMS analysis was 91%. Analysis condition B: Retention time=1.72 min; ESI-MS(+) m/z [M+2H]²⁺: 1018.2.

### Preparation of Compound 1498

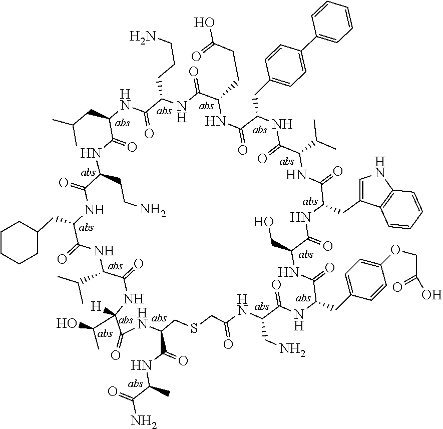 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CO)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1498 was prepared on a 50 μmol scale. The yield of the product was 18.6 mg, and its estimated purity by LCMS analysis was 96.2%. Analysis condition A: Retention time=1.49 min; ESI-MS(+) m/z [M+2H]²⁺: 973.3.

### Preparation of Compound 1499

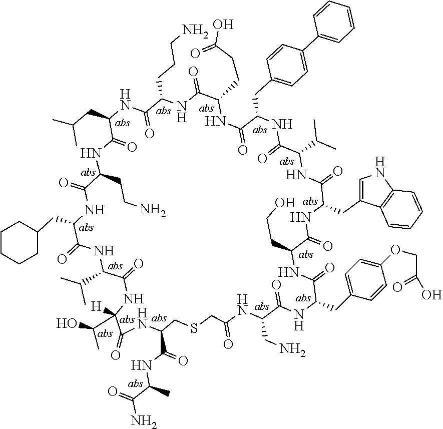 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CCO)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1499 was prepared on a 50 μmol scale. The yield of the product was 4.5 mg, and its estimated purity by LCMS analysis was 92%. Analysis condition A: Retention time=1.55, 1.6 min; ESI-MS(+) m/z [M+H]⁺: 1958.33, 1958.36.

### Preparation of Compound 1500

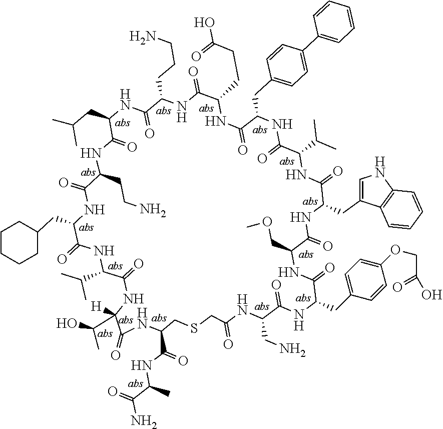 [SMILES: COC[C@@H]1NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC1=O]

Compound 1500 was prepared on a 50 μmol scale. The yield of the product was 4.6 mg, and its estimated purity by LCMS analysis was 100%. Analysis condition A: Retention time=1.74 min; ESI-MS(+) m/z [M+H]⁺: 1957.7.

### Preparation of Compound 1501

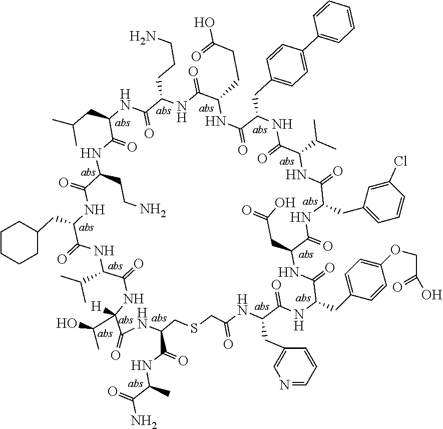 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1501 was prepared on a 50 μmol scale. The yield of the product was 8.9 mg, and its estimated purity by LCMS analysis was 93.4%. Analysis condition B: Retention time=1.61 min; ESI-MS(+) m/z [M+2H]²⁺: 1015.1.

### Preparation of Compound 1502

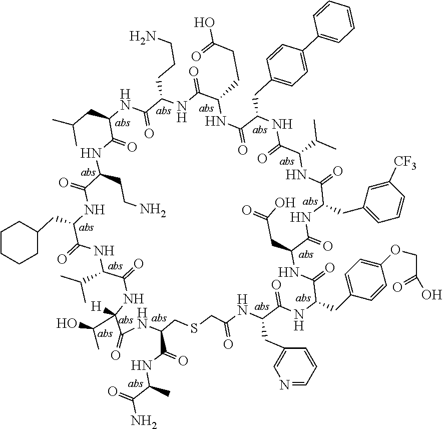 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2cccnc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1502 was prepared on a 50 μmol scale. The yield of the product was 11.8 mg, and its estimated purity by LCMS analysis was 93.5%. Analysis condition A: Retention time=1.46 min; ESI-MS(+) m/z [M+2H]²⁺: 1032.6.

### Preparation of Compound 1503

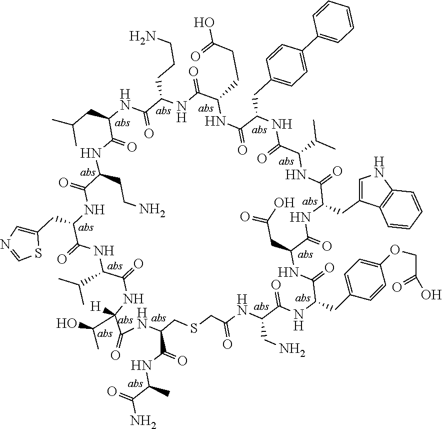 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cncs2)NC(=O)[C@H](CCN)NC1=O]

Compound 1503 was prepared on a 50 μmol scale. The yield of the product was 8.2 mg, and its estimated purity by LCMS analysis was 87.7%. Analysis condition B: Retention time=1.36 min; ESI-MS(+) m/z [M+H]⁺: 1972.9.

### Preparation of Compound 1504

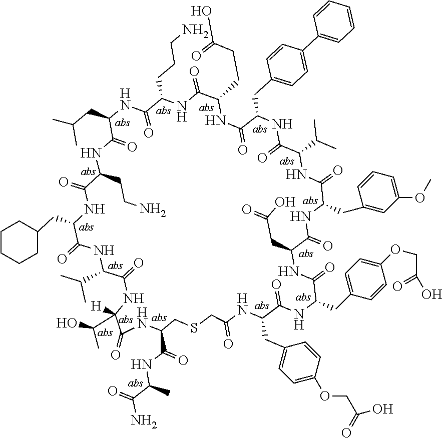 [SMILES: COc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1504 was prepared on a 50 μmol scale. The yield of the product was 7.2 mg, and its estimated purity by LCMS analysis was 94.6%. Analysis condition B: Retention time=1.69 min; ESI-MS(+) m/z [M+2H] 2:1050.

### Preparation of Compound 1505

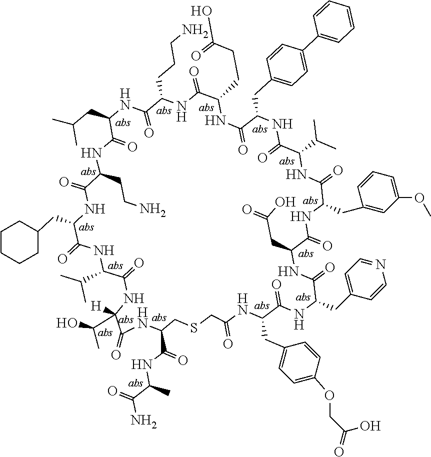 [SMILES: COc1cccc(C[C@@H]2NC(=O)[C@H](CC(=O)O)NC(=O)[C@H](Cc3ccncc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1505 was prepared on a 50 μmol scale. The yield of the product was 1.4 mg, and its estimated purity by LCMS analysis was 85.1%. Analysis condition B: Retention time=1.56 min; ESI-MS(+) m/z [M+2H]²⁺: 1013.2.

### Preparation of Compound 1506

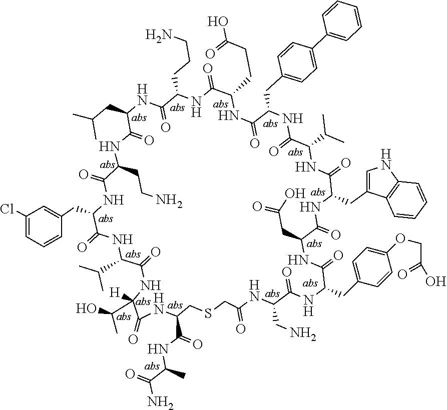 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(Cl)c2)NC(=O)[C@H](CCN)NC1=O]

Compound 1506 was prepared on a 50 μmol scale. The yield of the product was 9.6 mg, and its estimated purity by LCMS analysis was 85.6%. Analysis condition B: Retention time=1.56 min; ESI-MS(+) m/z [M+2H]²⁺: 1001.1.

### Preparation of Compound 1507

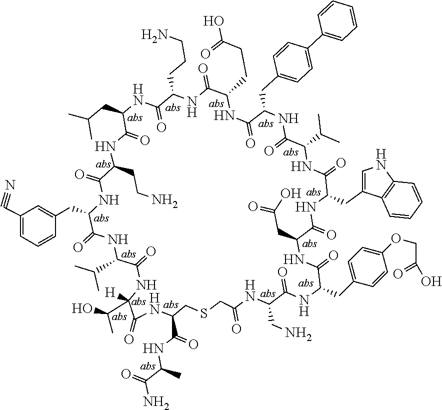 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(C#N)c2)NC(=O)[C@H](CCN)NC1=O]

Compound 1507 was prepared on a 50 μmol scale. The yield of the product was 6.9 mg, and its estimated purity by LCMS analysis was 95.3%. Analysis condition B: Retention time=1.43 min; ESI-MS(+) m/z [M+H]⁺: 1992.

### Preparation of Compound 1508

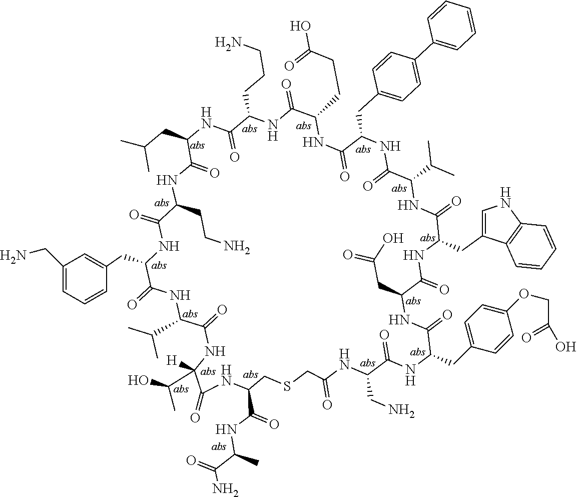 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(CN)c2)NC(=O)[C@H](CCN)NC1=O]

Compound 1508 was prepared on a 50 μmol scale. The yield of the product was 11.2 mg, and its estimated purity by LCMS analysis was 86.6%. Analysis condition B: Retention time=1.3 min; ESI-MS(+) m/z [M+H]⁺: 1994.8.

### Preparation of Compound 1509

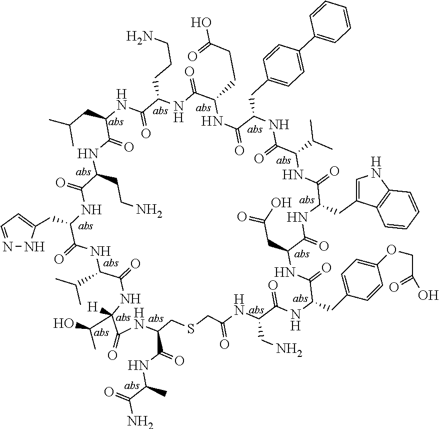 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccn[nH]2)NC(=O)[C@H](CCN)NC1=O]

Compound 1509 was prepared on a 50 μmol scale. The yield of the product was 1.6 mg, and its estimated purity by LCMS analysis was 100%. Analysis condition A: Retention time=1.3 min; ESI-MS(+) m/z [M+H]⁺: 1955.7.

### Preparation of Compound 1510

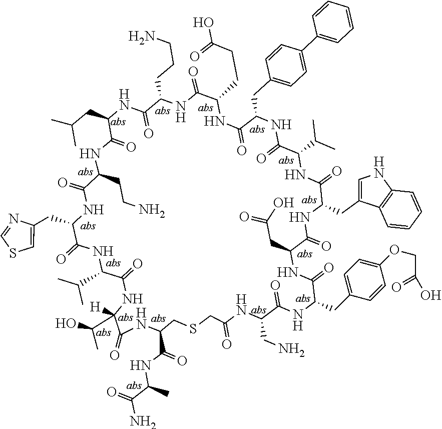 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cscn2)NC(=O)[C@H](CCN)NC1=O]

Compound 1510 was prepared on a 50 μmol scale. The yield of the product was 7.1 mg, and its estimated purity by LCMS analysis was 84.3%. Analysis condition B: Retention time=1.49 min; ESI-MS(+) m/z [M+2H]²⁺: 987.1.

### Preparation of Compound 1511

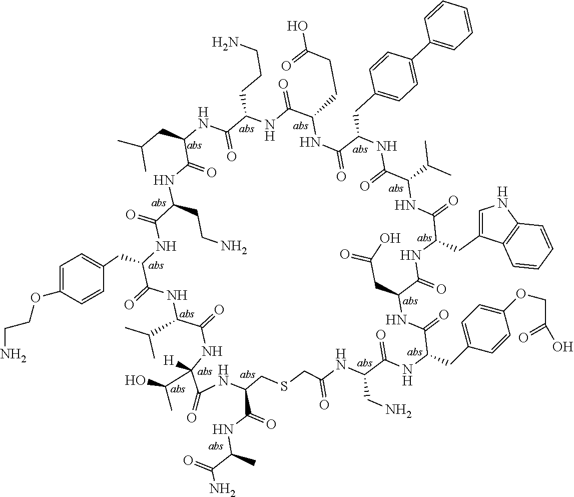 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(OCCN)cc2)NC(=O)[C@H](CCN)NC1=O]

Compound 1511 was prepared on a 50 μmol scale. The yield of the product was 4.9 mg, and its estimated purity by LCMS analysis was 97.3%. Analysis condition B: Retention time=1.37 min; ESI-MS(+) m/z [M+3H]³⁺: 675.8.

### Preparation of Compound 1512

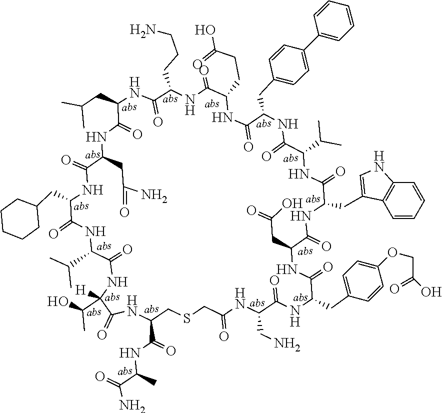 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CC(N)=O)NC1=O]

Compound 1512 was prepared on a 50 μmol scale. The yield of the product was 32.8 mg, and its estimated purity by LCMS analysis was 94.2%. Analysis condition A: Retention time=1.52 min; ESI-MS(+) m/z [M+H]⁺: 1985.9.

### Preparation of Compound 1513

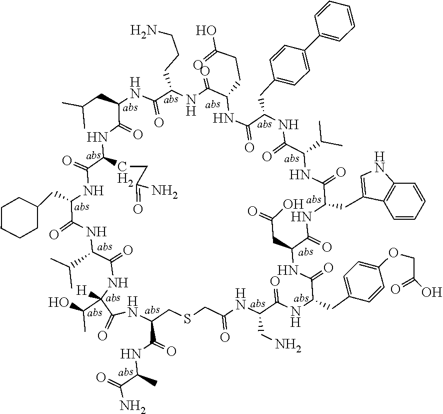 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCC(N)=O)NC1=O]

Compound 1513 was prepared on a 50 μmol scale. The yield of the product was 24.7 mg, and its estimated purity by LCMS analysis was 96%. Analysis condition A: Retention time=1.51 min; ESI-MS(+) m/z [M+2H]²⁺: 1000.9.

### Preparation of Compound 1514

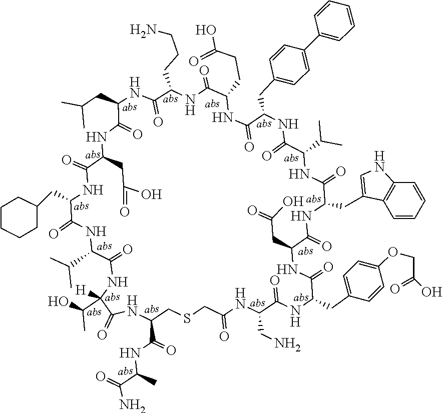 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CC(=O)O)NC1=O]

Compound 1514 was prepared on a 50 μmol scale. The yield of the product was 24.3 mg, and its estimated purity by LCMS analysis was 87.6%. Analysis condition B: Retention time=1.56 min; ESI-MS(+) m/z [M+H]⁺: 1988.2.

### Preparation of Compound 1515

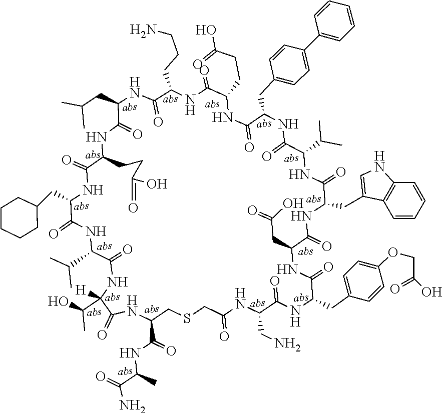 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCC(=O)O)NC1=O]

Compound 1515 was prepared on a 50 μmol scale. The yield of the product was 17.4 mg, and its estimated purity by LCMS analysis was 96.3%. Analysis condition B: Retention time=1.58 min; ESI-MS(+) m/z [M+3H]³⁺: 668.

### Preparation of Compound 1516

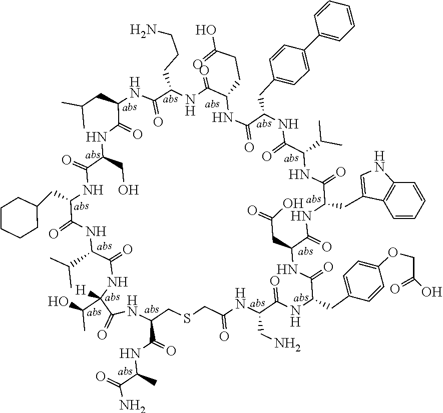 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CO)NC1=O]

Compound 1516 was prepared on a 50 μmol scale. The yield of the product was 8.8 mg, and its estimated purity by LCMS analysis was 90.1%. Analysis condition A: Retention time=1.56 min; ESI-MS(+) m/z [M+H]⁺: 1959.

### Preparation of Compound 1517

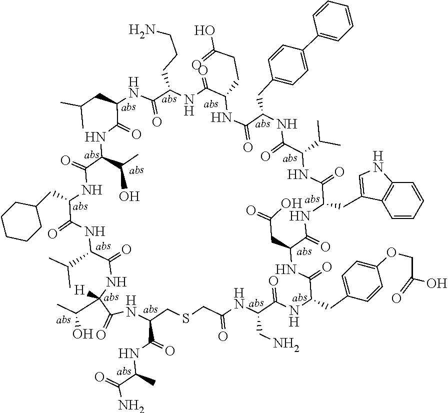 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H]([C@@H](C)O)NC1=O]

Compound 1517 was prepared on a 50 μmol scale. The yield of the product was 17.6 mg, and its estimated purity by LCMS analysis was 91.5%. Analysis condition B: Retention time=1.7 min; ESI-MS(+) m/z [M+H]⁺: 1974.2.

### Preparation of Compound 1518

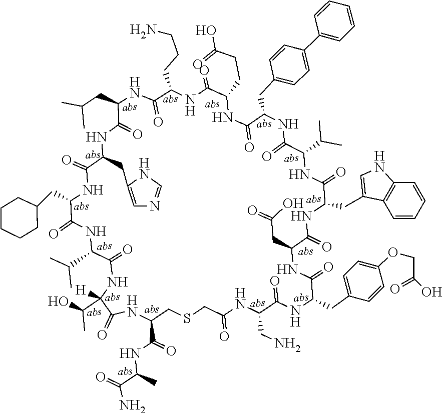 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1518 was prepared on a 50 μmol scale. The yield of the product was 21.3 mg, and its estimated purity by LCMS analysis was 100%. Analysis condition A: Retention time=1.49 min; ESI-MS(+) m/z [M+2H]²⁺: 1005.1.

### Preparation of Compound 1519

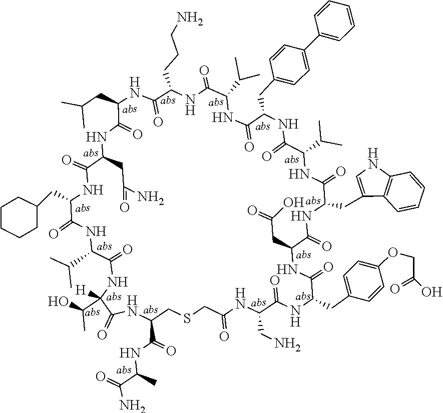 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CC(N)=O)NC1=O]

Compound 1519 was prepared on a 50 μmol scale. The yield of the product was 15.2 mg, and its estimated purity by LCMS analysis was 73.3%. Analysis condition A: Retention time=1.72 min; ESI-MS(+) m/z [M+H]⁺: 1955.7.

### Preparation of Compound 1520

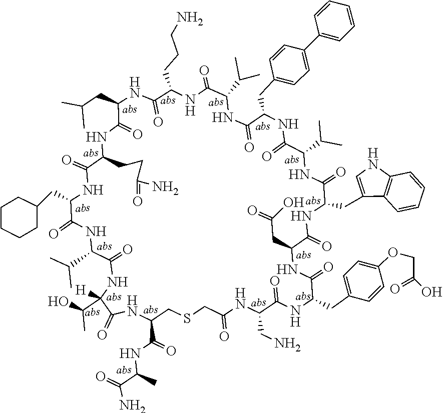 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCC(N)=O)NC1=O]

Compound 1520 was prepared on a 50 μmol scale. The yield of the product was 13.2 mg, and its estimated purity by LCMS analysis was 92.1%. Analysis condition B: Retention time=1.62 min; ESI-MS(+) m/z [M+H]⁺: 1971.

### Preparation of Compound 1521

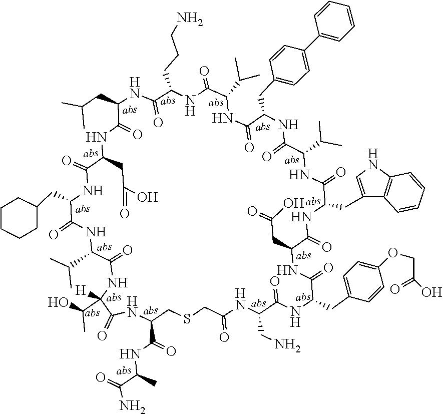 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CC(=O)O)NC1=O]

Compound 1521 was prepared on a 50 μmol scale. The yield of the product was 12.8 mg, and its estimated purity by LCMS analysis was 93.1%. Analysis condition B: Retention time=1.66 min; ESI-MS(+) m/z [M+H]⁺: 1957.

### Preparation of Compound 1522

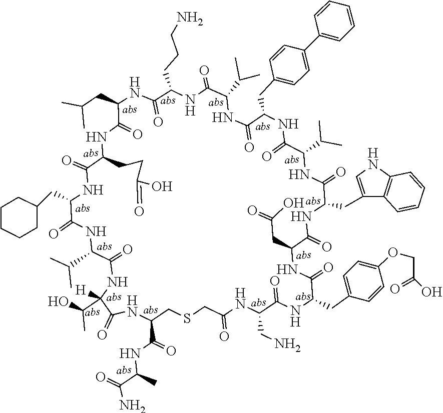 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCC(=O)O)NC1=O]

Compound 1522 was prepared on a 50 μmol scale. The yield of the product was 4.6 mg, and its estimated purity by LCMS analysis was 90.6%. Analysis condition B: Retention time=1.66 min; ESI-MS(+) m/z [M+2H]²⁺: 1972.1.

### Preparation of Compound 1523

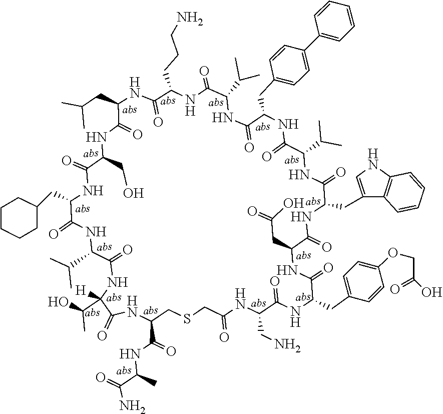 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CO)NC1=O]

Compound 1523 was prepared on a 50 μmol scale. The yield of the product was 7.2 mg, and its estimated purity by LCMS analysis was 95.2%. Analysis condition B: Retention time=1.69 min; ESI-MS(+) m/z [M+2H]²⁺: 1930.2.

### Preparation of Compound 1524

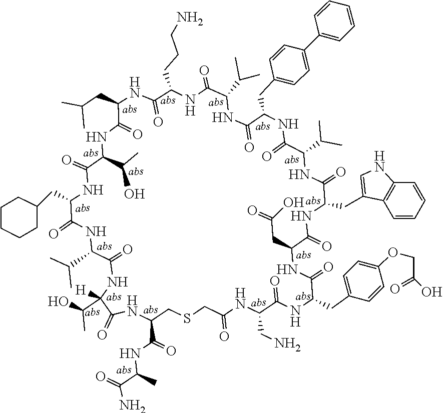 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H]([C@@H](C)O)NC1=O]

Compound 1524 was prepared on a 50 μmol scale. The yield of the product was 10.9 mg, and its estimated purity by LCMS analysis was 92.3%. Analysis condition B: Retention time=1.66 min; ESI-MS(+) m/z [M+2H]²⁺: 1944.

### Preparation of Compound 1525

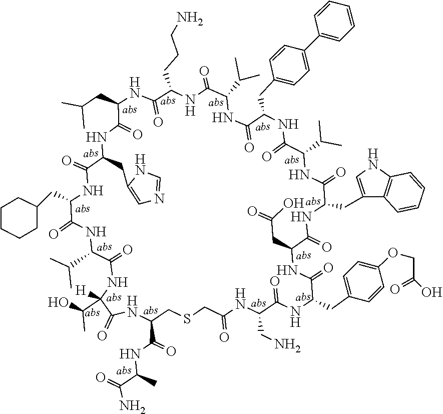 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1525 was prepared on a 50 μmol scale. The yield of the product was 12.8 mg, and its estimated purity by LCMS analysis was 92.1%. Analysis condition B: Retention time=1.59 min; ESI-MS(+) m/z [M+2H]²⁺: 660.3.

### Preparation of Compound 1526

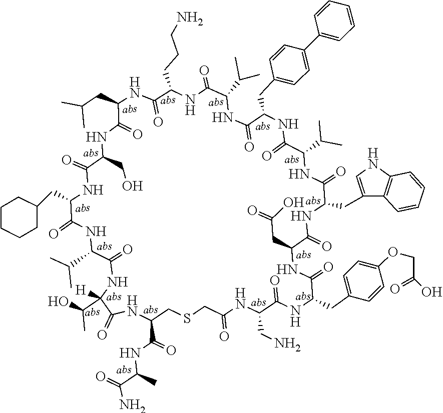 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CO)NC1=O]

Compound 1526 was prepared on a 50 μmol scale. The yield of the product was 6.9 mg, and its estimated purity by LCMS analysis was 86.5%. Analysis condition B: Retention time=1.73 min; ESI-MS(+) m/z [M+2H]²⁺: 965.1.

### Preparation of Compound 1527

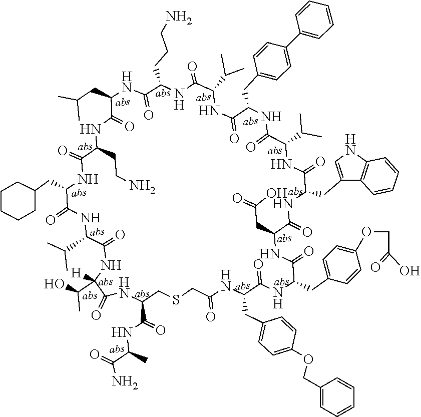 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCc3ccccc3)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1527 was prepared on a 50 μmol scale. The yield of the product was 7.1 mg, and its estimated purity by LCMS analysis was 95.1%. Analysis condition B: Retention time=1.84 min; ESI-MS(+) m/z [M+2H]²⁺: 1055.2.

### Preparation of Compound 1528

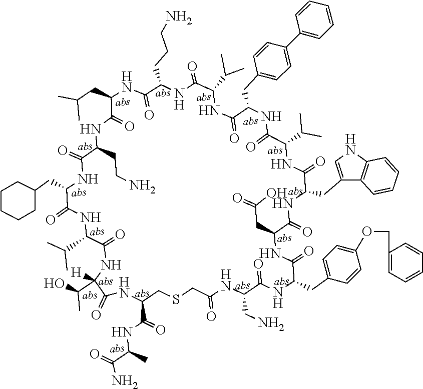 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCc3ccccc3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1528 was prepared on a 50 μmol scale. The yield of the product was 7.6 mg, and its estimated purity by LCMS analysis was 97.1%. Analysis condition B: Retention time=1.8 min; ESI-MS(+) m/z [M+H]⁺: 1973.9.

### Preparation of Compound 1529

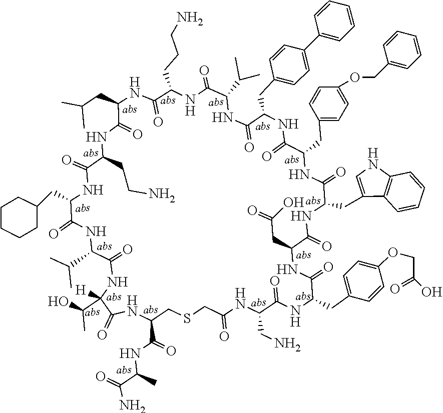 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](Cc2ccc(OCc3ccccc3)cc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1529 was prepared on a 50 μmol scale. The yield of the product was 12.7 mg, and its estimated purity by LCMS analysis was 96.9%. Analysis condition A: Retention time=1.86 min; ESI-MS(+) m/z [M+2H]²⁺: 1048.9.

### Preparation of Compound 1530

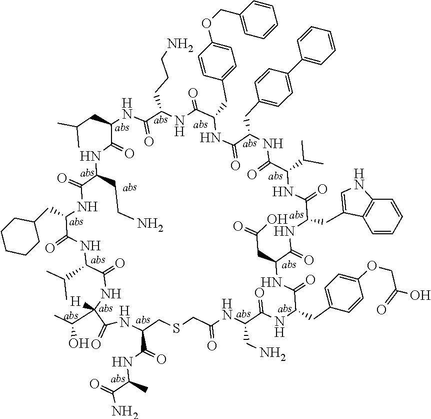 [SMILES: CC(C)C[C@H]1NC(=O)[C@H](CCCN)NC(=O)[C@H](Cc2ccc(OCc3ccccc3)cc2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1530 was prepared on a 50 μmol scale. The yield of the product was 12.6 mg, and its estimated purity by LCMS analysis was 95.2%. Analysis condition B: Retention time=1.77 min; ESI-MS(+) m/z [M+2H]²⁺: 1049.1.

### Preparation of Compound 1531

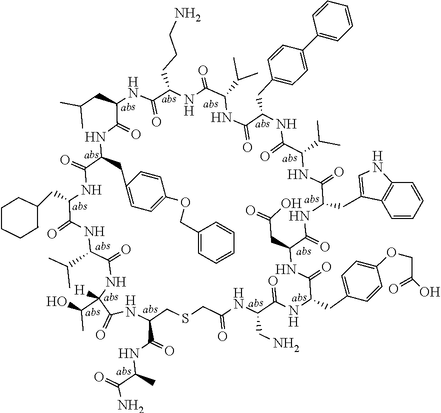 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2ccc(OCc3ccccc3)cc2)NC1=O]

Compound 1531 was prepared on a 50 μmol scale. The yield of the product was 14.3 mg, and its estimated purity by LCMS analysis was 92.5%. Analysis condition A: Retention time=2.18 min; ESI-MS(+) m/z [M+2H]²⁺: 1048.2.

### Preparation of Compound 1532

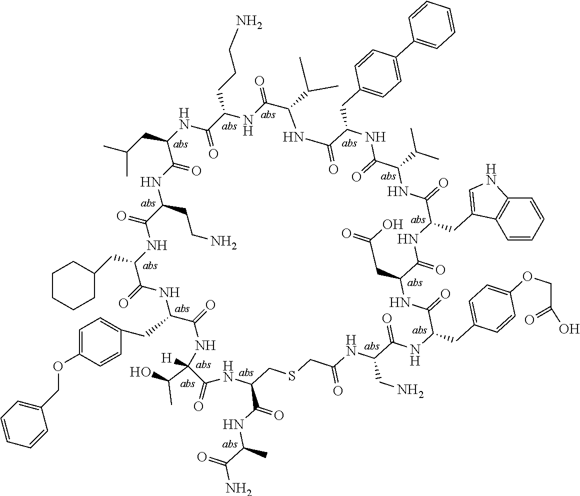 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](Cc2ccc(OCc3ccccc3)cc2)NC(=O)[C@H](CC2CCCCC2)NC(=O)[C@H](CCN)NC1=O]

Compound 1532 was prepared on a 50 μmol scale. The yield of the product was 13.4 mg, and its estimated purity by LCMS analysis was 98%. Analysis condition A: Retention time=1.98 min; ESI-MS(+) m/z [M+2H]²⁺: 1049.2.

### Preparation of Compound 1533

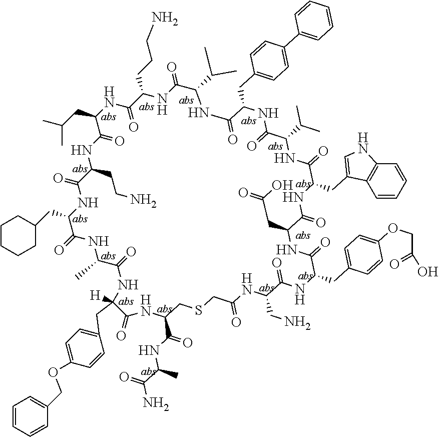 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](Cc2ccc(OCc3ccccc3)cc2)NC(=O)[C@H](C)NC(=O)[C@H](CC2CCCCC2)NC(=O)[C@H](CCN)NC1=O]

Compound 1533 was prepared on a 50 μmol scale. The yield of the product was 7.5 mg, and its estimated purity by LCMS analysis was 95.3%. Analysis condition A: Retention time=1.86 min; ESI-MS(+) m/z [M+2H]²⁺: 1048.2.

### Preparation of Compound 1534

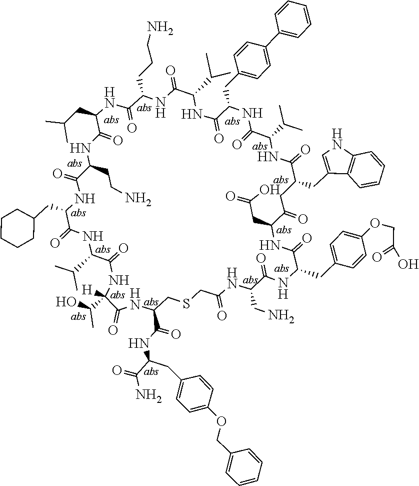 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)CC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](Cc2ccc(OCc3ccccc3)cc2)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1534 was prepared on a 50 μmol scale. The yield of the product was 4 mg, and its estimated purity by LCMS analysis was 97.2%. Analysis condition A: Retention time=1.92 min; ESI-MS(+) m/z [M+2H]²⁺: 1062.2.

### Preparation of Compound 1535

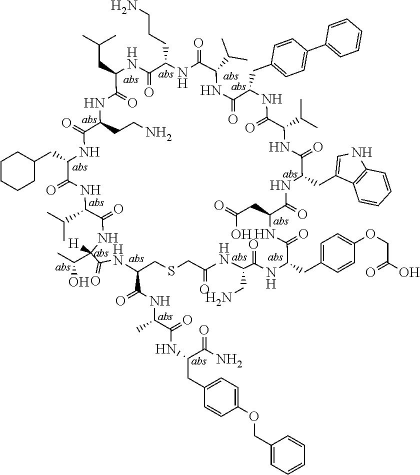 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(=O)N[C@@H](Cc2ccc(OCc3ccccc3)cc2)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1535 was prepared on a 50 μmol scale. The yield of the product was 3.9 mg, and its estimated purity by LCMS analysis was 90.3%. Analysis condition B: Retention time=1.65 min; ESI-MS(+) m/z [M+3H]³⁺: 733.

### Preparation of Compound 1536

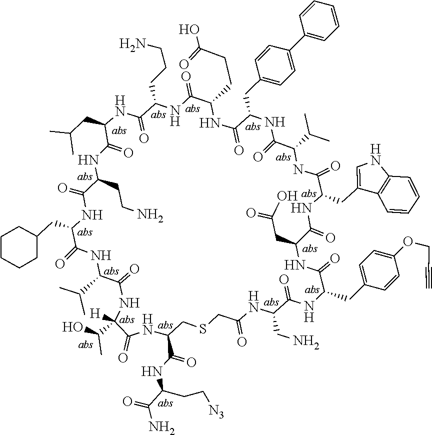

Compound 1536 was prepared on a 50 μmol scale. The yield of the product was 13.6 mg, and its estimated purity by LCMS analysis was 92.9%. Analysis condition B: Retention time=1.56 min; ESI-MS(+) m/z [M+3H]³⁺: 670.1.

### Preparation of Compound 1537

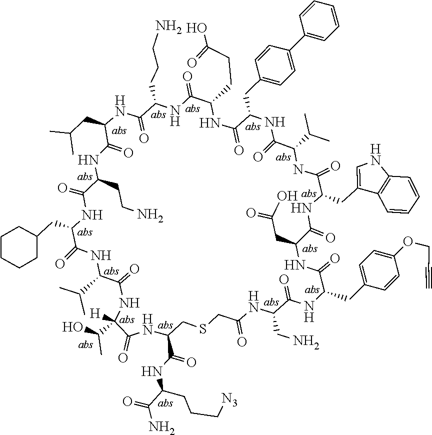

Compound 1537 was prepared on a 50 μmol scale. The yield of the product was 10.9 mg, and its estimated purity by LCMS analysis was 93.4%. Analysis condition A: Retention time=1.79 min; ESI-MS(+) m/z [M+2H]²⁺: 1011.

### Preparation of Compound 1538

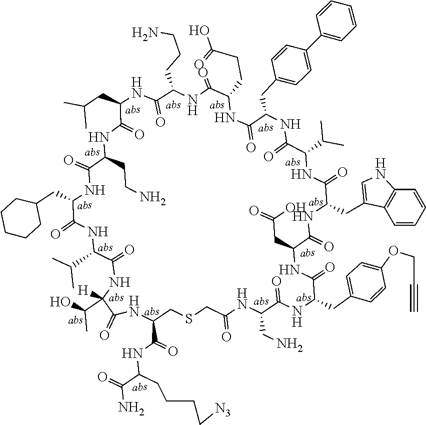 [SMILES: C#CCOc1ccc(C[C@@H]2NC(=O)[C@H](CN)NC(=O)CSC[C@@H](C(=O)NC(CCCCN=[N+]=[N-])C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)cc1]

Compound 1538 was prepared on a 50 μmol scale. The yield of the product was 12.4 mg, and its estimated purity by LCMS analysis was 90.2%. Analysis condition A: Retention time=1.72 min; ESI-MS(+) m/z [M+2H]²⁺: 1018.6.

### Preparation of Compound 1539

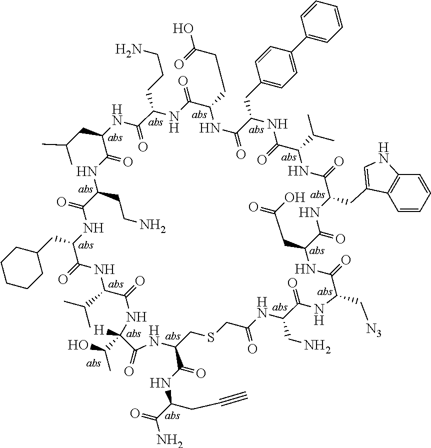 [SMILES: C#CC[C@H](NC(=O)[C@@H]1CSCC(=O)N[C@@H](CN)C(=O)N[C@@H](CN=[N+]=[N-])C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CCC(=O)O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1539 was prepared on a 50 μmol scale. The yield of the product was 13.6 mg, and its estimated purity by LCMS analysis was 77.9%. Analysis condition A: Retention time=1.63 min; ESI-MS(+) m/z [M+H]⁺: 1887.2.

### Preparation of Compound 1540

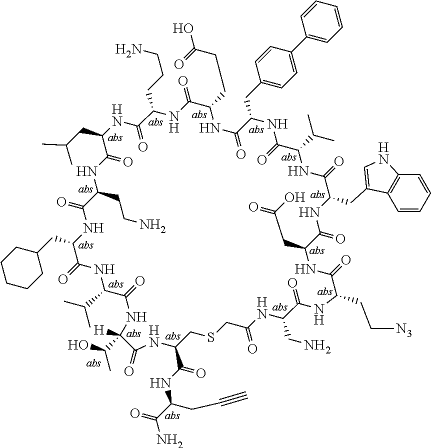 [SMILES: C#CC[C@H](NC(=O)[C@@H]1CSCC(=O)N[C@@H](CN)C(=O)N[C@@H](CCN=[N+]=[N-])C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](CCC(=O)O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1540 was prepared on a 50 μmol scale. The yield of the product was 14.5 mg, and its estimated purity by LCMS analysis was 75.8%. Analysis condition A: Retention time=1.66 min; ESI-MS(+) m/z [M+H]⁺: 1901.1.

### Preparation of Compound 1541

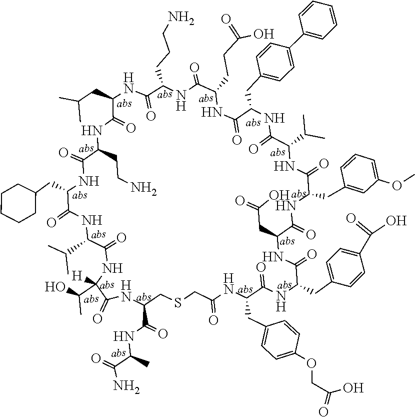 [SMILES: COc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1541 was prepared on a 50 μmol scale. The yield of the product was 5.3 mg, and its estimated purity by LCMS analysis was 92.6%. Analysis condition A: Retention time=1.39 min; ESI-MS(+) m/z [M+2H]²⁺: 1034.1.

### Preparation of Compound 1542

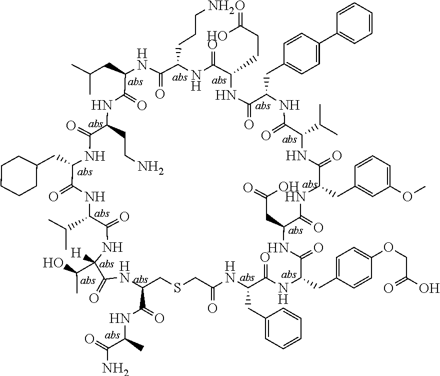 [SMILES: COc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1542 was prepared on a 50 μmol scale. The yield of the product was 8 mg, and its estimated purity by LCMS analysis was 93.8%. Analysis condition B: Retention time=1.58 min; ESI-MS(+) m/z [M+2H]²⁺: 1013.2.

### Preparation of Compound 1543

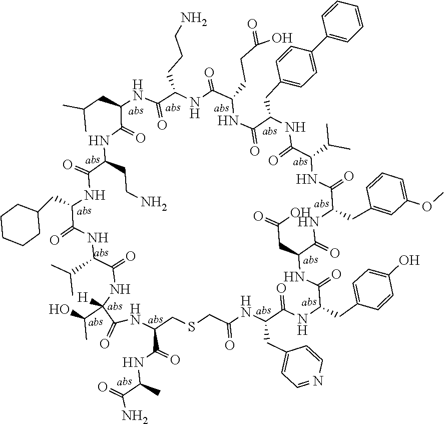 [SMILES: COc1cccc(C[C@@H]2NC(=O)[C@H](CC(=O)O)NC(=O)[C@H](Cc3ccc(O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1543 was prepared on a 50 μmol scale. The yield of the product was 3.6 mg, and its estimated purity by LCMS analysis was 97.6%. Analysis condition B: Retention time=1.52 min; ESI-MS(+) m/z [M+H]⁺: 1967.3.

### Preparation of Compound 1544

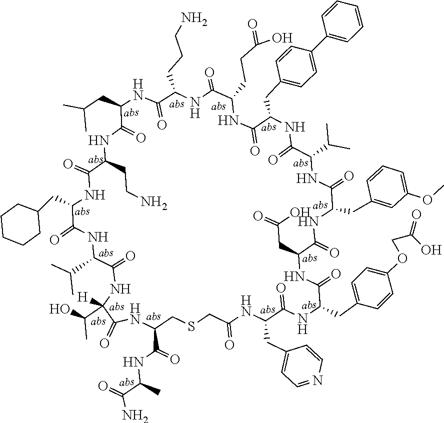 [SMILES: COc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1544 was prepared on a 50 μmol scale. The yield of the product was 6.7 mg and its estimated purity by LCMS analysis was 93.1%. Analysis condition A: Retention time=1.45 min; ESI-MS(+) m/z [M+2H]²⁺: 1013.2.

### Preparation of Compound 1545

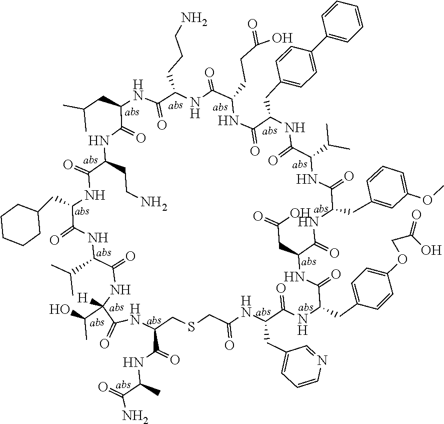 [SMILES: COc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1545 was prepared on a 50 μmol scale. The yield of the product was 5.3 mg, and its estimated purity by LCMS analysis was 97.1%. Analysis condition B: Retention time=1.61 min; ESI-MS(+) m/z [M+3H]³⁺: 675.8.

### Preparation of Compound 1546

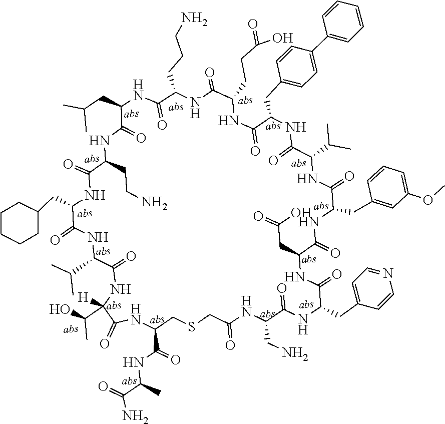 [SMILES: COc1cccc(C[C@@H]2NC(=O)[C@H](CC(=O)O)NC(=O)[C@H](Cc3ccncc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1546 was prepared on a 50 μmol scale. The yield of the product was 9.5 mg, and its estimated purity by LCMS analysis was 100%. Analysis condition A: Retention time=1.61 min; ESI-MS(+) m/z [M+H]⁺: 1891.

### Preparation of Compound 1547

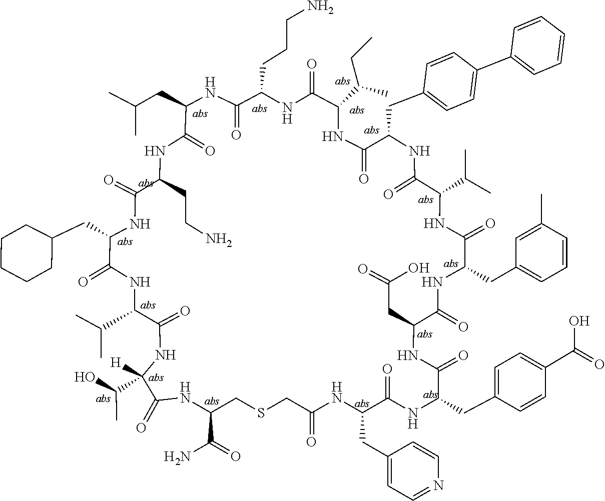 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1547 was prepared on a 50 μmol scale. The yield of the product was 3.1 mg, and its estimated purity by LCMS analysis was 98.2%. Analysis condition B: Retention time=1.64 min; ESI-MS(+) m/z [M+H]⁺: 1891.8.

### Preparation of Compound 1548

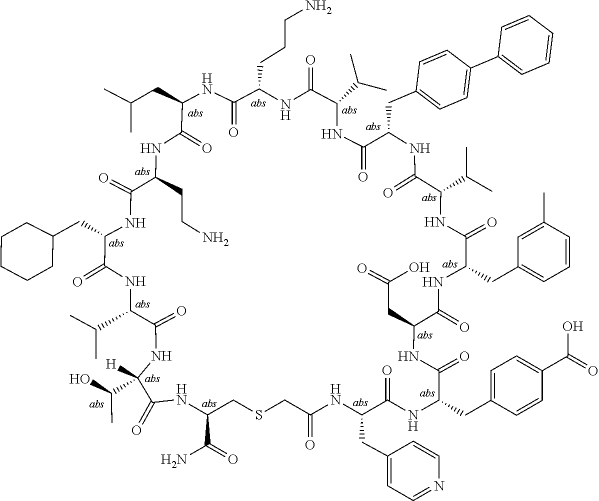 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1548 was prepared on a 50 μmol scale. The yield of the product was 5 mg, and its estimated purity by LCMS analysis was 86%. Analysis condition A: Retention time=1.67 min; ESI-MS(+) m/z [M+2H]²⁺: 940.3.

### Preparation of Compound 1549

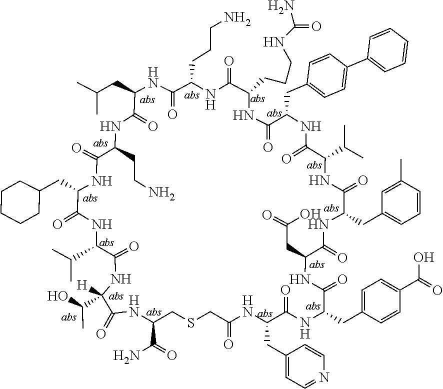 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1549 was prepared on a 50 μmol scale. The yield of the product was 10.3 mg, and its estimated purity by LCMS analysis was 96%. Analysis condition A: Retention time=1.59 min; ESI-MS(+) m/z [M+H]⁺: 1936.2.

### Preparation of Compound 1550

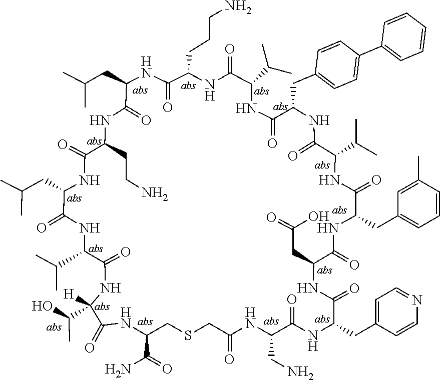 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1550 was prepared on a 50 μmol scale. The yield of the product was 2.6 mg, and its estimated purity by LCMS analysis was 98%. Analysis condition B: Retention time=1.23 min; ESI-MS(+) m/z [M+3H]³⁺: 578.7.

### Preparation of Compound 1551

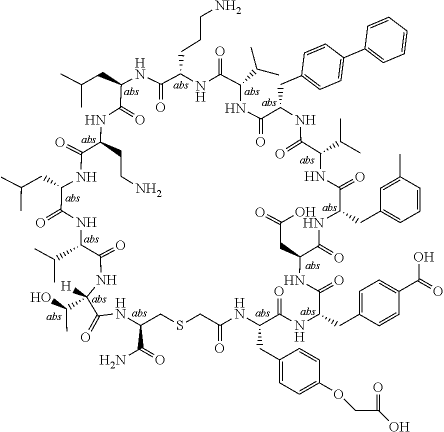 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1551 was prepared on a 50 μmol scale. The yield of the product was 1.6 mg, and its estimated purity by LCMS analysis was 87.6%. Analysis condition B: Retention time=1.65, 1.68 min; ESI-MS(+) m/z [M+H]⁺: 1912.

### Preparation of Compound 1552

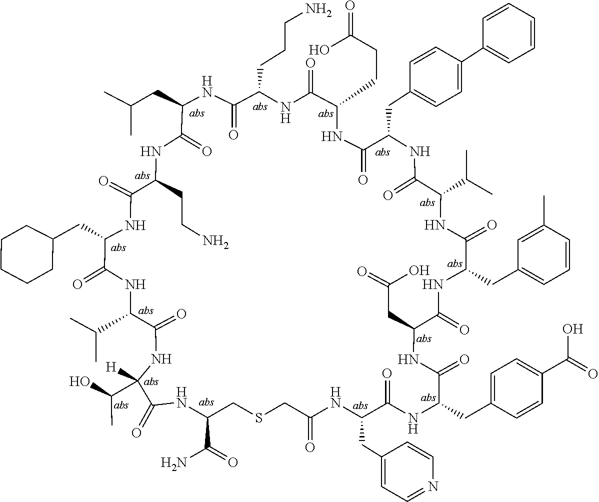 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1552 was prepared on a 50 μmol scale. The yield of the product was 7.6 mg, and its estimated purity by LCMS analysis was 87.1%. Analysis condition A: Retention time=1.49 min; ESI-MS(+) m/z [M+H]⁺: 1908.

### Preparation of Compound 1553

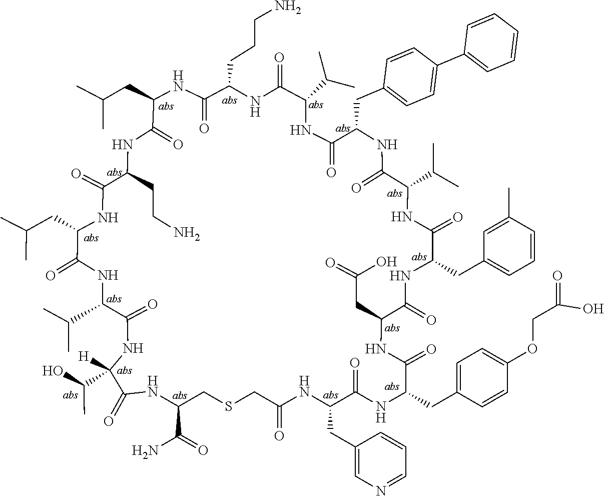 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1553 was prepared on a 50 μmol scale. The yield of the product was 2.9 mg, and its estimated purity by LCMS analysis was 86.1%. Analysis condition A: Retention time=1.67 min; ESI-MS(+) m/z [M+H]⁺: 1867.7.

### Preparation of Compound 1554

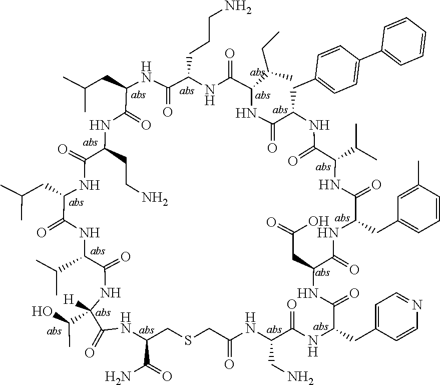 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccncc2)NC(=O)[C@H](CN)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1554 was prepared on a 50 μmol scale. The yield of the product was 1.6 mg, and its estimated purity by LCMS analysis was 80.7%. Analysis condition A: Retention time=1.68 min; ESI-MS(+) m/z [M+H]⁺: 1746.7.

### Preparation of Compound 1555

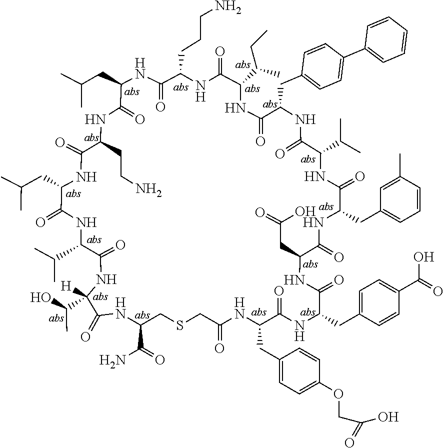 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1555 was prepared on a 50 μmol scale. The yield of the product was 4.8 mg, and its estimated purity by LCMS analysis was 84.2%. Analysis condition A: Retention time=1.77 min; ESI-MS(+) m/z [M+H]⁺: 1924.8.

### Preparation of Compound 1556

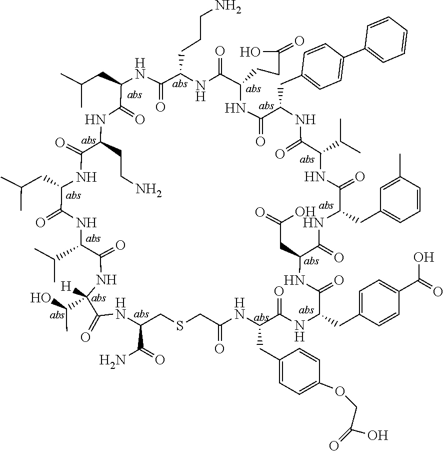 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 1556 was prepared on a 50 μmol scale. The yield of the product was 9.7 mg, and its estimated purity by LCMS analysis was 91%. Analysis condition B: Retention time=1.63 min; ESI-MS(+) m/z [M+H]⁺: 1940.7.

### Preparation of Compound 1557

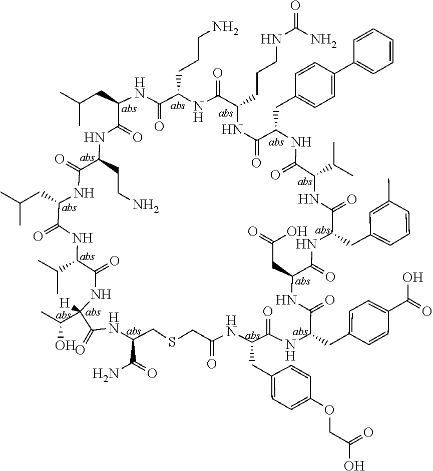 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1557 was prepared on a 50 μmol scale. The yield of the product was 6.9 mg, and its estimated purity by LCMS analysis was 91.6%. Analysis condition A: Retention time=1.49 min; ESI-MS(+) m/z [M+H]⁺: 1968.7.

### Preparation of Compound 1558

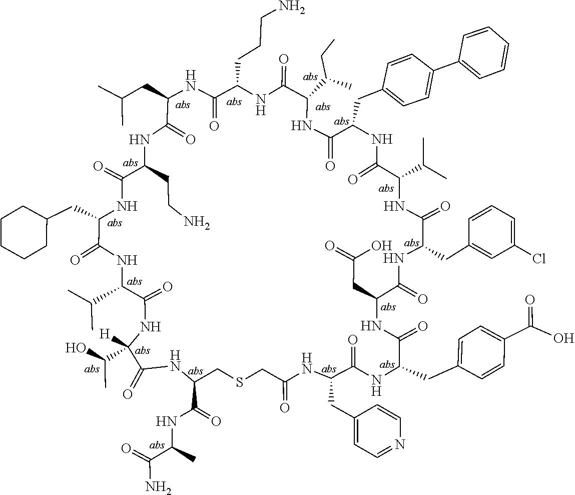 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1558 was prepared on a 50 μmol scale. The yield of the product was 1.5 mg, and its estimated purity by LCMS analysis was 91.1%. Analysis condition B: Retention time=1.62 min; ESI-MS(+) m/z [M+H]⁺: 1983.1.

### Preparation of Compound 1559

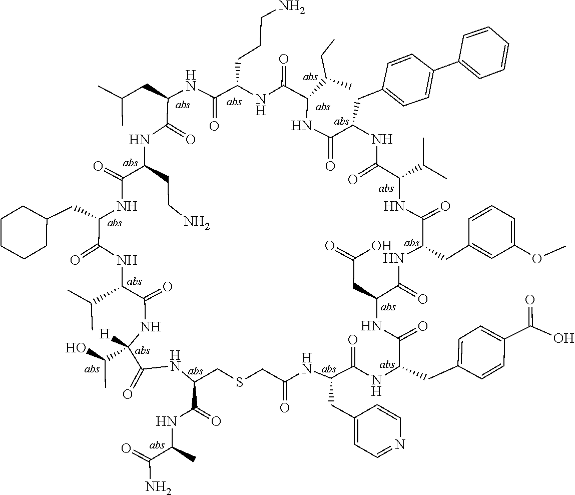 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(OC)c2)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1559 was prepared on a 50 μmol scale. The yield of the product was 2.9 mg, and its estimated purity by LCMS analysis was 98.1%. Analysis condition B: Retention time=1.53 min; ESI-MS(+) m/z [M+H]⁺: 1980.3.

### Preparation of Compound 1560

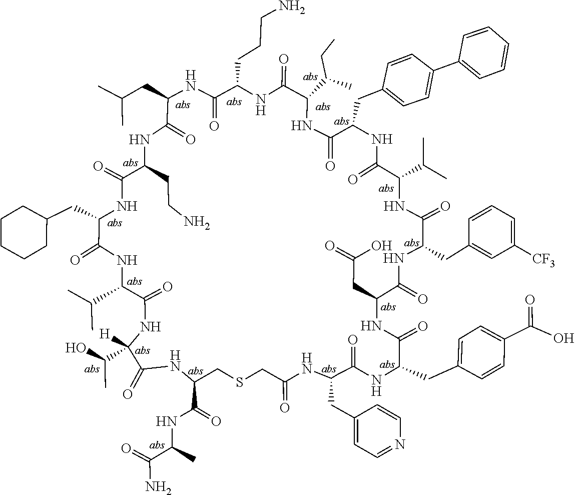 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1560 was prepared on a 50 μmol scale. The yield of the product was 6.1 mg, and its estimated purity by LCMS analysis was 96.2%. Analysis condition A: Retention time=1.86 min; ESI-MS(+) m/z [M+2H]²⁺: 1009.

### Preparation of Compound 1561

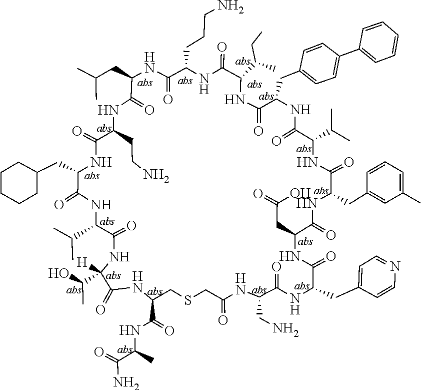 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccncc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1561 was prepared on a 50 μmol scale. The yield of the product was 2.9 mg, and its estimated purity by LCMS analysis was 90.9%. Analysis condition A: Retention time=1.82 min; ESI-MS(+) m/z [M+H]⁺: 1858.

### Preparation of Compound 1562

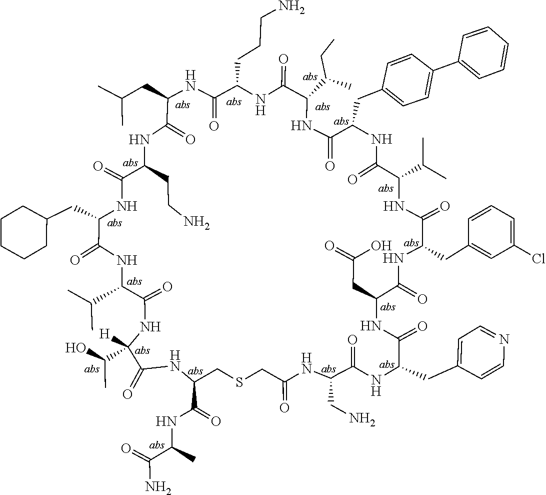 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccncc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1562 was prepared on a 50 μmol scale. The yield of the product was 2.6 mg, and its estimated purity by LCMS analysis was 94.9%. Analysis condition A: Retention time=1.86 min; ESI-MS(+) m/z [M+H]⁺: 1878.

### Preparation of Compound 1563

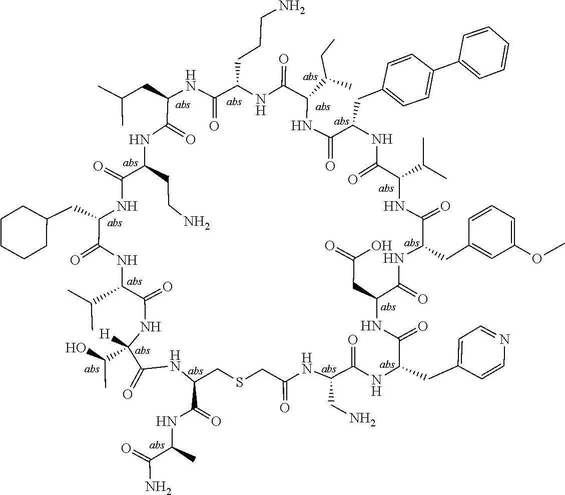 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(OC)c2)NC(=O)[C@H](CC(=O)O)NC(=O)[C@H](Cc2ccncc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1563 was prepared on a 50 μmol scale. The yield of the product was 1.4 mg, and its estimated purity by LCMS analysis was 92.1%. Analysis condition B: Retention time=1.47 min; ESI-MS(+) m/z [M+H]⁺: 1873.7.

### Preparation of Compound 1564

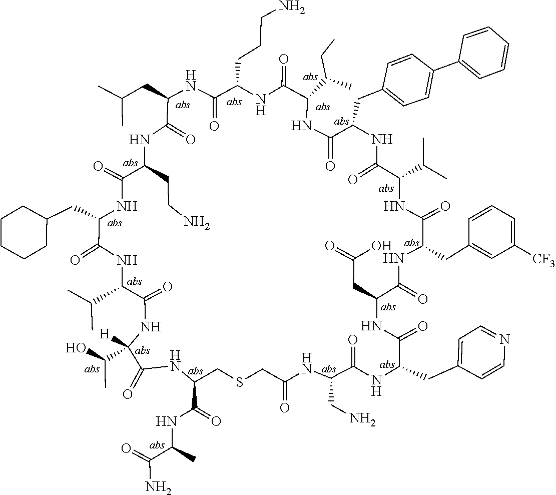 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccncc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1564 was prepared on a 50 μmol scale. The yield of the product was 11.5 mg, and its estimated purity by LCMS analysis was 100%. Analysis condition A: Retention time=2.07 min; ESI-MS(+) m/z [M+H]⁺: 1912.3.

### Preparation of Compound 1565

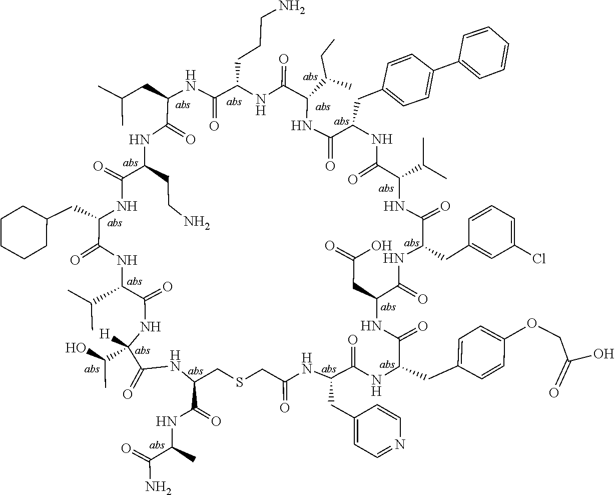 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1565 was prepared on a 50 μmol scale. The yield of the product was 2.5 mg, and its estimated purity by LCMS analysis was 91.3%. Analysis condition B: Retention time=1.65 min; ESI-MS(+) m/z [M+3H]³⁺: 672.2.

### Preparation of Compound 1566

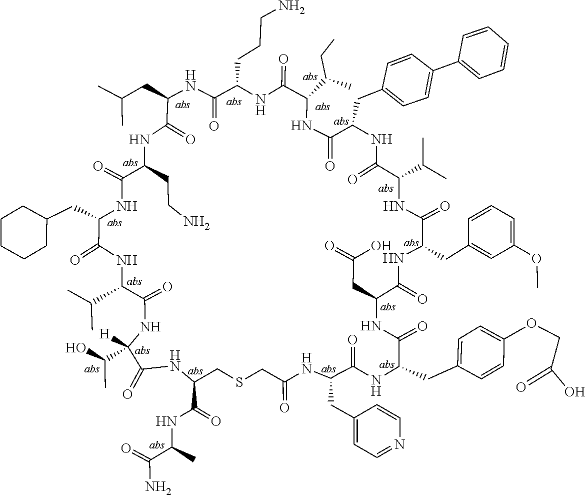 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(OC)c2)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1566 was prepared on a 50 μmol scale. The yield of the product was

3.6 mg, and its estimated purity by LCMS analysis was 92.7%. Analysis condition A: Retention time=1.74 min; ESI-MS(+) m/z [M+2H]²⁺: 1005.1.

### Preparation of Compound 1567

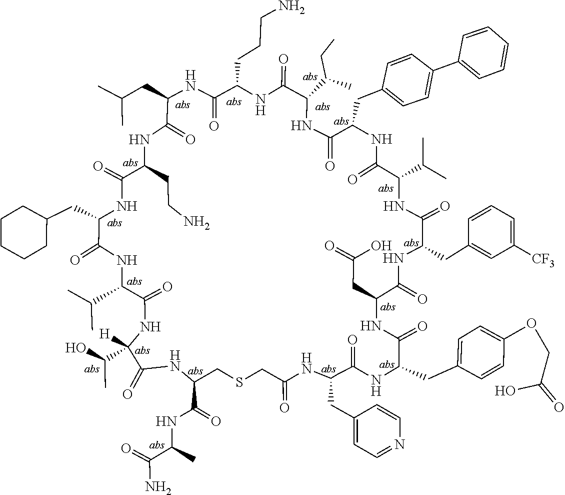 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1567 was prepared on a 50 μmol scale. The yield of the product was 2.3 mg, and its estimated purity by LCMS analysis was 93.8%. Analysis condition B: Retention time=1.64 min; ESI-MS(+) m/z [M+2H]²⁺: 1024.2.

### Preparation of Compound 1568

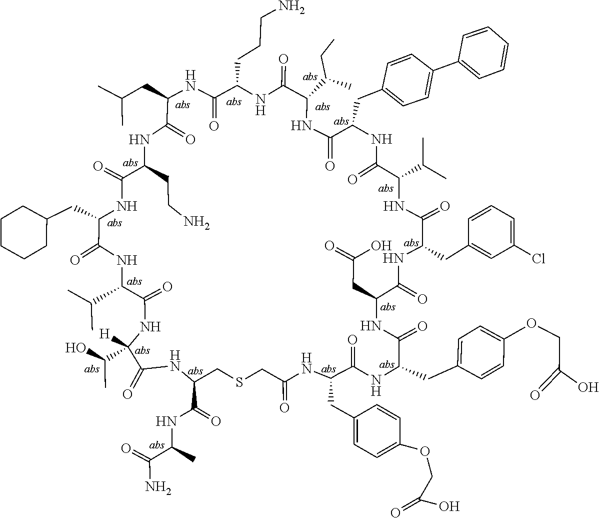 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)CN)NC(=O)[C@@H](CC(C)C)NC(=O)[C@H](CCCN)NC1=O]

Compound 1568 was prepared on a 50 μmol scale. The yield of the product was 2.8 mg, and its estimated purity by LCMS analysis was 95.3%. Analysis condition A: Retention time=1.94 min; ESI-MS(+) m/z [M+2H]²⁺: 1044.2.

### Preparation of Compound 1569

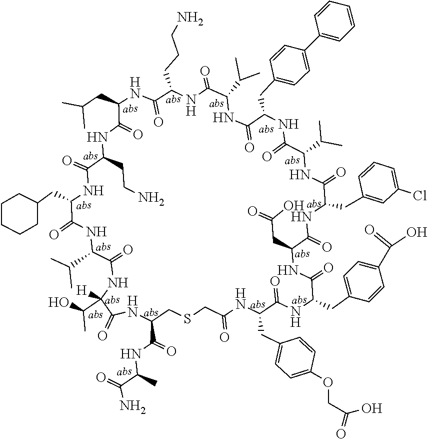 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1569 was prepared on a 50 μmol scale. The yield of the product was 2.6 mg, and its estimated purity by LCMS analysis was 90.6%. Analysis condition A: Retention time=2.11 min; ESI-MS(+) m/z [M+2H]²⁺: 1022.

### Preparation of Compound 1570

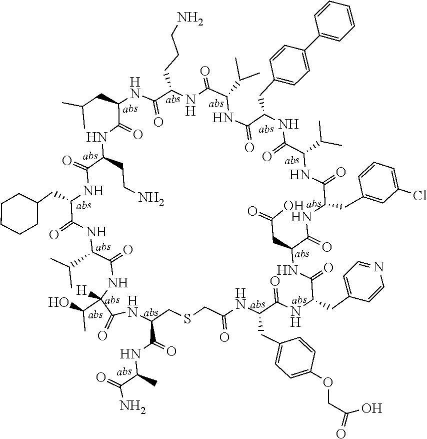 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1570 was prepared on a 50 μmol scale. The yield of the product was 2.7 mg, and its estimated purity by LCMS analysis was 100%. Analysis condition A: Retention time=2.09 min; ESI-MS(+) m/z [M+H]⁺: 1999.8.

### Preparation of Compound 1571

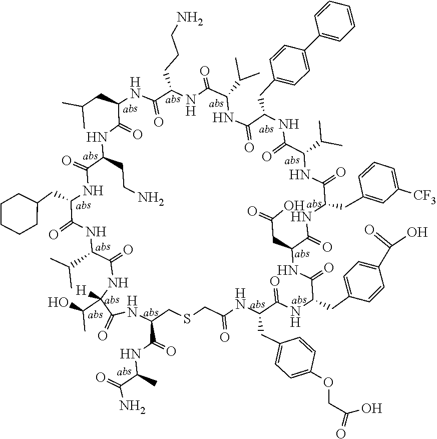 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1571 was prepared on a 50 μmol scale. The yield of the product was 167 mg, and its estimated purity by LCMS analysis was 90.8%. Analysis condition B: Retention time=1.84 min; ESI-MS(+) m/z [M+2H]²⁺: 1039.

### Preparation of Compound 1572

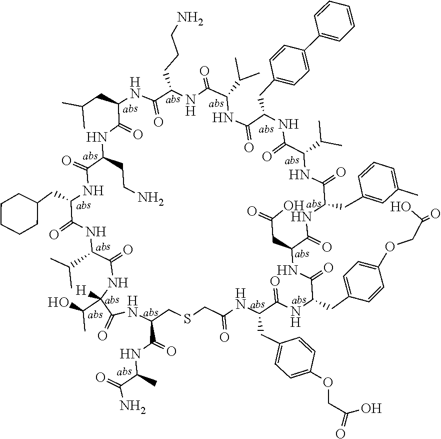 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1572 was prepared on a 50 μmol scale. The yield of the product was 2.9 mg, and its estimated purity by LCMS analysis was 92.9%. Analysis condition B: Retention time=1.78 min; ESI-MS(+) m/z [M+2H]²⁺: 1027.1.

### Preparation of Compound 1573

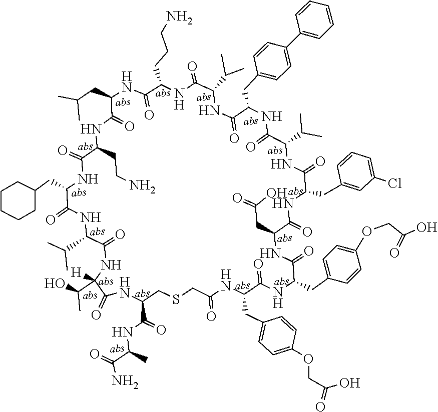 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1573 was prepared on a 50 μmol scale. The yield of the product was 1.9 mg, and its estimated purity by LCMS analysis was 98.4%. Analysis condition B: Retention time=1.83 min; ESI-MS(+) m/z [M+2H]²⁺: 1037.

### Preparation of Compound 1574

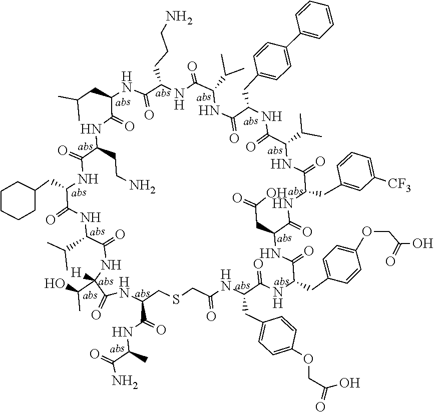 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1574 was prepared on a 50 μmol scale. The yield of the product was 3.6 mg, and its estimated purity by LCMS analysis was 91.3%. Analysis condition B: Retention time=1.84 min; ESI-MS(+) m/z [M+2H]²⁺: 1054.

### Preparation of Compound 1575

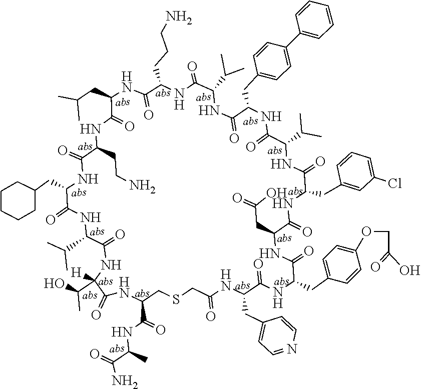 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1575 was prepared on a 50 μmol scale. The yield of the product was 4.6 mg, and its estimated purity by LCMS analysis was 91%. Analysis condition A: Retention time=1.74 min; ESI-MS(+) m/z [M+H]⁺: 1999.6.

### Preparation of Compound 1576

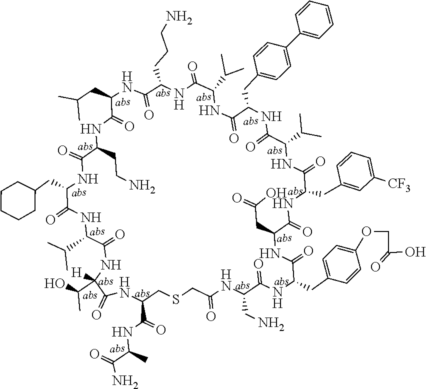 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1576 was prepared on a 50 μmol scale. The yield of the product was 2 mg, and its estimated purity by LCMS analysis was 92.8%. Analysis condition B: Retention time=1.68 min; ESI-MS(+) m/z [M+H]⁺: 1970.9.

### Preparation of Compound 1577

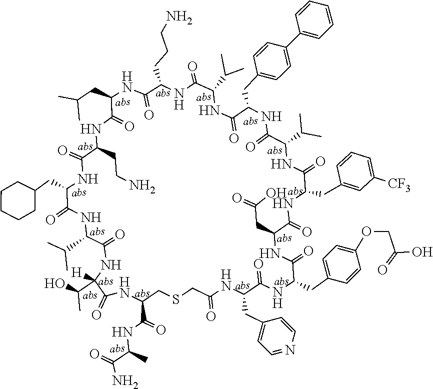 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1577 was prepared on a 50 μmol scale. The yield of the product was 3.4 mg, and its estimated purity by LCMS analysis was 96.2%. Analysis condition B: Retention time=1.68 min; ESI-MS(+) m/z [M+3H]³⁺: 678.6.

### Preparation of Compound 1578

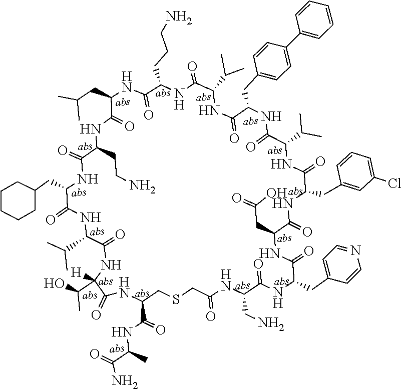 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cccc(Cl)c2)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1578 was prepared on a 50 μmol scale. The yield of the product was 0.7 mg, and its estimated purity by LCMS analysis was 67.3%. Analysis condition A: Retention time=1.95 min; ESI-MS(+) m/z [M+2H]²⁺: 932.8.

### Preparation of Compound 1579

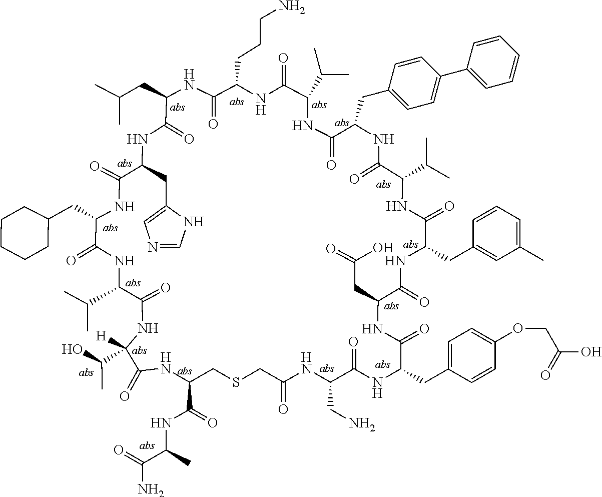 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1579 was prepared on a 50 μmol scale. The yield of the product was 16.1 mg, and its estimated purity by LCMS analysis was 100%. Analysis condition A: Retention time=1.75 min; ESI-MS(+) m/z [M+H]⁺: 1954.2.

### Preparation of Compound 1580

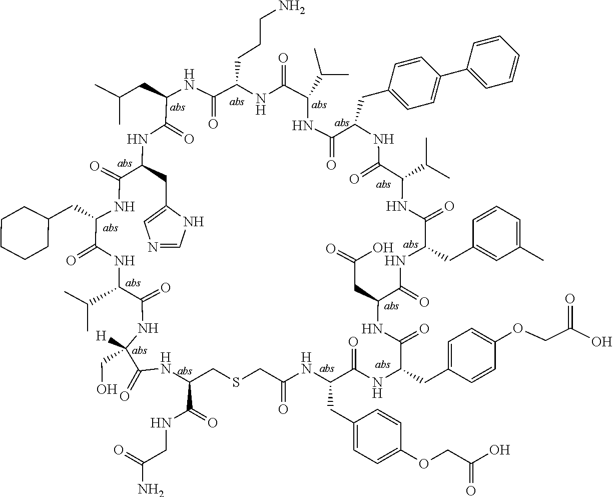 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(N)=O)NC(=O)[C@H](CO)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1580 was prepared on a 50 μmol scale. The yield of the product was 9.7 mg, and its estimated purity by LCMS analysis was 94.8%. Analysis condition A: Retention time=1.62 min; ESI-MS(+) m/z [M+2H]²⁺: 1038.

### Preparation of Compound 1581

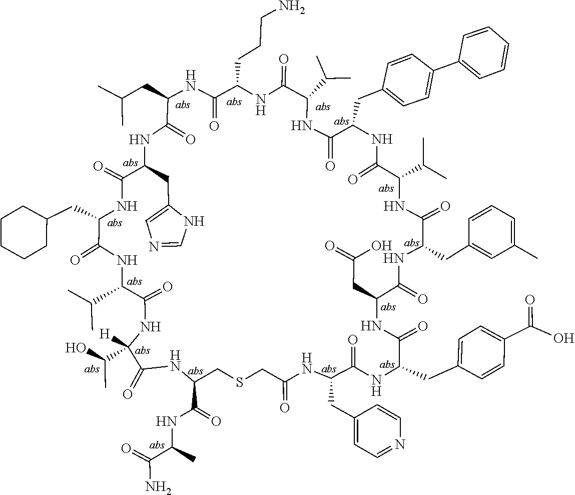 [SMILES: Cc1cccc(C[C@@H]2NC(=O)[C@H](CC(=O)O)NC(=O)[C@H](Cc3ccc(C(=O)O)cc3)NC(=O)C[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](C(C)c3[nH]cnc3C)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1581 was prepared on a 50 μmol scale. The yield of the product was 18.3 mg, and its estimated purity by LCMS analysis was 98.3%. Analysis condition A: Retention time=1.79 min; ESI-MS(+) m/z [M+H]⁺: 1987.

### Preparation of Compound 1582

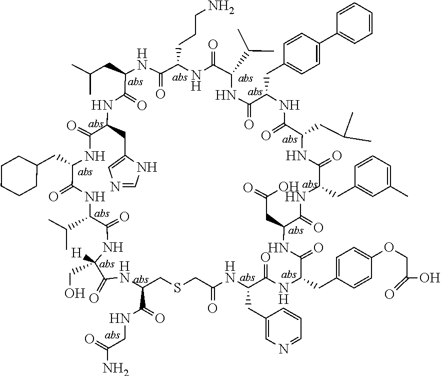 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)NCC(N)=O)NC(=O)[C@H](CO)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1582 was prepared on a 50 μmol scale. The yield of the product was 15.2 mg, and its estimated purity by LCMS analysis was 98.9%. Analysis condition A: Retention time=1.77 min; ESI-MS(+) m/z [M+2H]²⁺: 1002.1.

### Preparation of Compound 1583

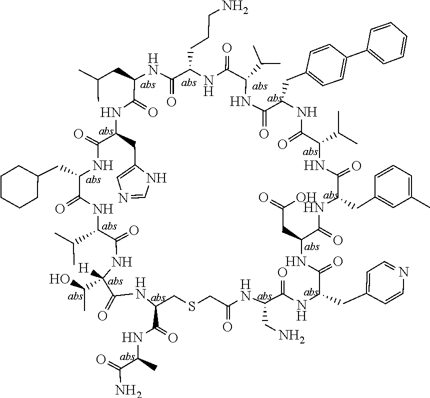 [SMILES: Cc1cccc(C[C@@H]2NC(=O)[C@H](CC(=O)O)NC(=O)[C@H](Cc3ccncc3)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](C(C)c3[nH]cnc3C)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1583 was prepared on a 50 μmol scale. The yield of the product was 10.9 mg, and its estimated purity by LCMS analysis was 84.4%. Analysis condition B: Retention time=1.42 min; ESI-MS(+) m/z [M+H]⁺: 1881.

### Preparation of Compound 1584

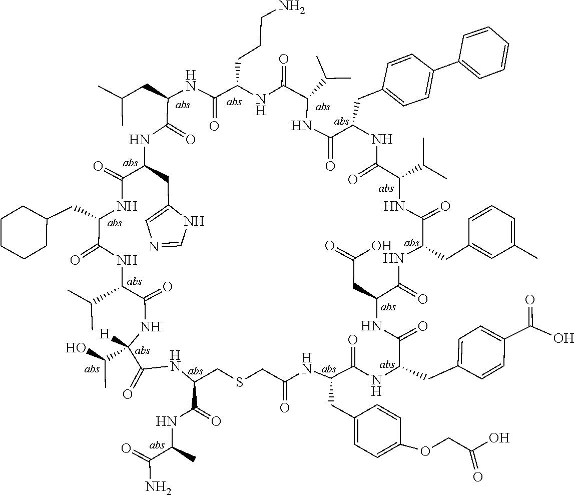 [SMILES: Cc1cccc(C[C@@H]2NC(=O)[C@H](CC(=O)O)NC(=O)[C@H](Cc3ccc(C(=O)O)cc3)NC(=O)C[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](C(C)c3[nH]cnc3C)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1584 was prepared on a 50 μmol scale. The yield of the product was 10.9 mg, and its estimated purity by LCMS analysis was 90.6%. Analysis condition A: Retention time=1.7 min; ESI-MS(+) m/z [M+2H]²⁺: 1030.1.

### Preparation of Compound 1585

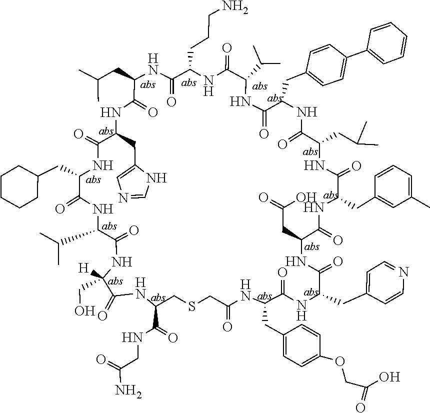 [SMILES: Cc1cccc(C[C@@H]2NC(=O)[C@H](CC(=O)O)NC(=O)[C@H](Cc3ccncc3)NC(=O)C[C@H](Cc3ccc(OCC(=O)O)cc3)NC(=O)CSC[C@@H](C(=O)NCC(N)=O)NC(=O)[C@H](CO)NC(=O)[C@H](C(C)C)NC(=O)[C@H](CC3CCCCC3)NC(=O)[C@H](C(C)c3[nH]cnc3C)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1585 was prepared on a 50 μmol scale. The yield of the product was 16.1 mg, and its estimated purity by LCMS analysis was 95.9%. Analysis condition A: Retention time=1.87 min; ESI-MS(+) m/z [M+2H]²⁺: 1002.1.

### Preparation of Compound 1586

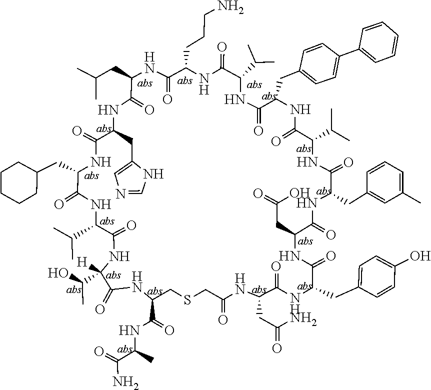 [SMILES: Cc1cccc(C[C@@H]2NC(=O)[C@H](CC(=O)O)NC(=O)[C@H](Cc3ccc(O)cc3)NC(=O)C[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](C(C)c3[nH]cnc3C)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1586 was prepared on a 50 μmol scale. The yield of the product was 4.1 mg, and its estimated purity by LCMS analysis was 91.7%. Analysis condition B: Retention time=1.64 min; ESI-MS(+) m/z [M+H]⁺: 1924.

### Preparation of Compound 1587

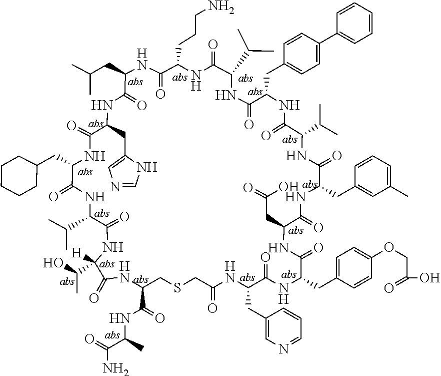 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1587 was prepared on a 50 μmol scale. The yield of the product was 22.6 mg, and its estimated purity by LCMS analysis was 94.7%. Analysis condition B: Retention time=1.59 min; ESI-MS(+) m/z [M+2H]²⁺: 1009.

### Preparation of Compound 1588

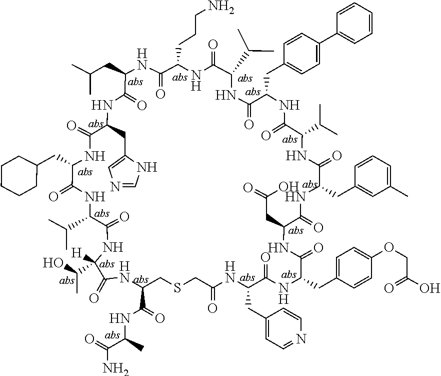 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1588 was prepared on a 50 μmol scale. The yield of the product was 15.6 mg, and its estimated purity by LCMS analysis was 97.5%. Analysis condition A: Retention time=1.69 min; ESI-MS(+) m/z [M+2H]²⁺: 1009.

### Preparation of Compound 1589

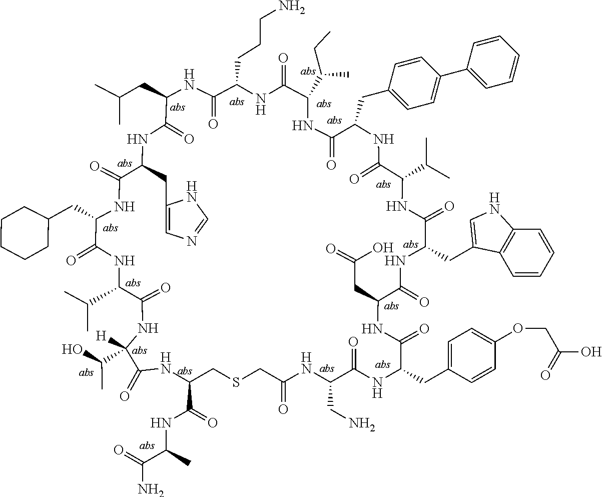 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC1=O]

Compound 1589 was prepared on a 50 μmol scale. The yield of the product was 31.9 mg, and its estimated purity by LCMS analysis was 97.1%. Analysis condition B: Retention time=1.54 min; ESI-MS(+) m/z [M+H]⁺: 1993.2.

### Preparation of Compound 1590

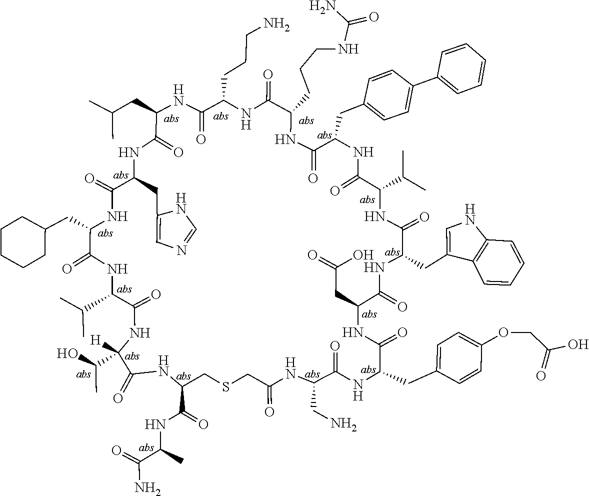 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1590 was prepared on a 50 μmol scale. The yield of the product was 23.4 mg, and its estimated purity by LCMS analysis was 95.1%. Analysis condition B: Retention time=1.53 min; ESI-MS(+) m/z [M+2H]²⁺: 1019.9.

### Preparation of Compound 1591

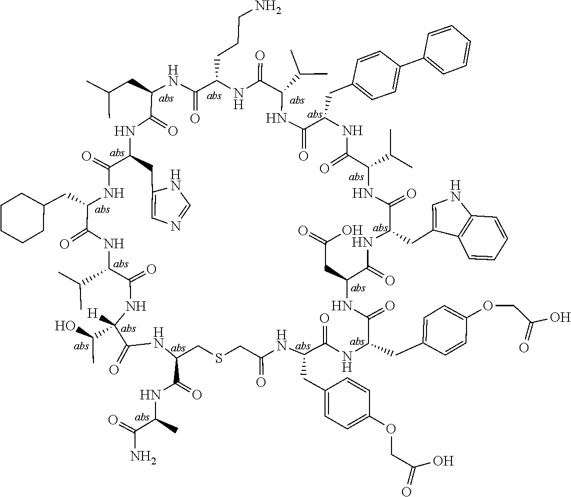 [SMILES: Cc1nc[nH]c1C(C)[C@@H]1NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1591 was prepared on a 50 μmol scale. The yield of the product was 36.6 mg, and its estimated purity by LCMS analysis was 87.2%. Analysis condition A: Retention time=1.66 min; ESI-MS(+) m/z [M+2H]²⁺: 1058.2.

### Preparation of Compound 1592

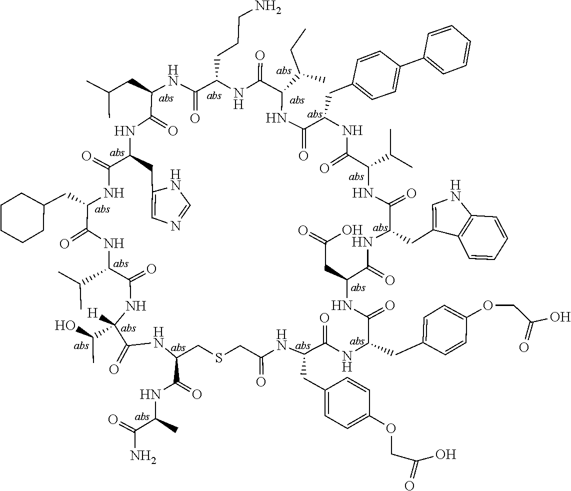 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)c2[nH]cnc2C)NC(=O)[C@@H](CC(C)C)NC(=O)[C@H](CCCN)NC1=O]

Compound 1592 was prepared on a 50 μmol scale. The yield of the product was 38.2 mg, and its estimated purity by LCMS analysis was 97%. Analysis condition A: Retention time=1.7 min; ESI-MS(+) m/z [M+2H]²⁺: 1065.1.

### Preparation of Compound 1593

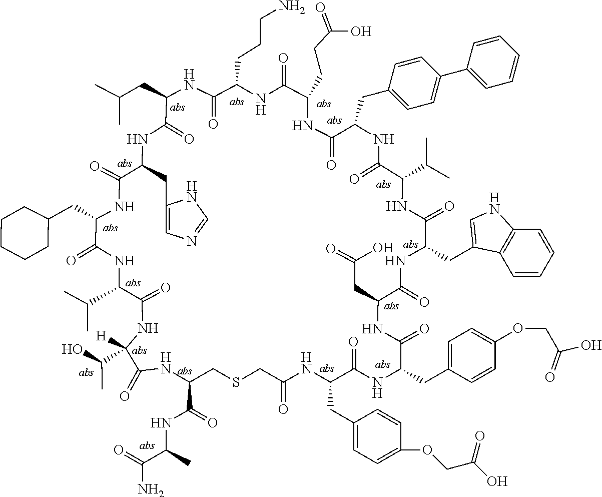 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1593 was prepared on a 50 μmol scale. The yield of the product was 21.3 mg, and its estimated purity by LCMS analysis was 95.7%. Analysis condition B: Retention time=1.66 min; ESI-MS(+) m/z [M+2H]²⁺: 1073.4.

### Preparation of Compound 1594

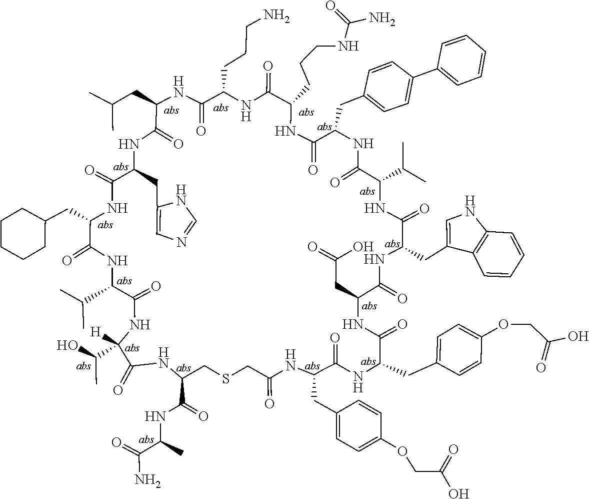 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1594 was prepared on a 50 μmol scale. The yield of the product was 25.8 mg, and its estimated purity by LCMS analysis was 97.8%. Analysis condition B: Retention time=1.65 min; ESI-MS(+) m/z [M+2H]²⁺: 1086.9.

### Preparation of Compound 1595

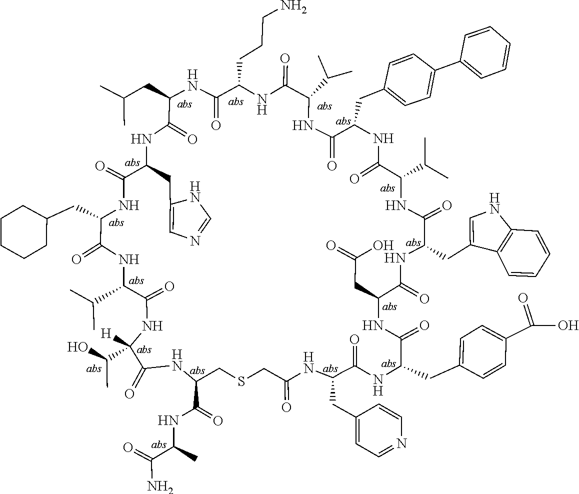 [SMILES: Cc1nc[nH]c1C(C)[C@@H]1NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1595 was prepared on a 50 μmol scale. The yield of the product was 48.4 mg, and its estimated purity by LCMS analysis was 93.4%. Analysis condition B: Retention time=1.54 min; ESI-MS(+) m/z [M+3H]³⁺: 671.

### Preparation of Compound 1596

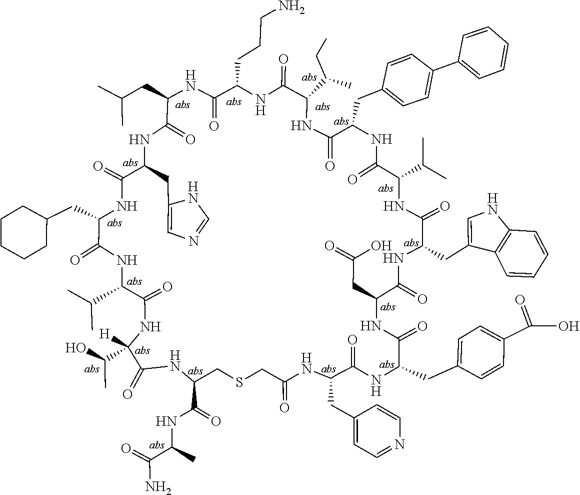 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)c2[nH]cnc2C)NC(=O)[C@@H](CC(C)C)NC(=O)[C@H](CCCN)NC1=O]

Compound 1596 was prepared on a 50 μmol scale. The yield of the product was 38.6 mg, and its estimated purity by LCMS analysis was 98%. Analysis condition A: Retention time=1.74 min; ESI-MS(+) m/z [M+3H]³⁺: 676.2.

### Preparation of Compound 1597

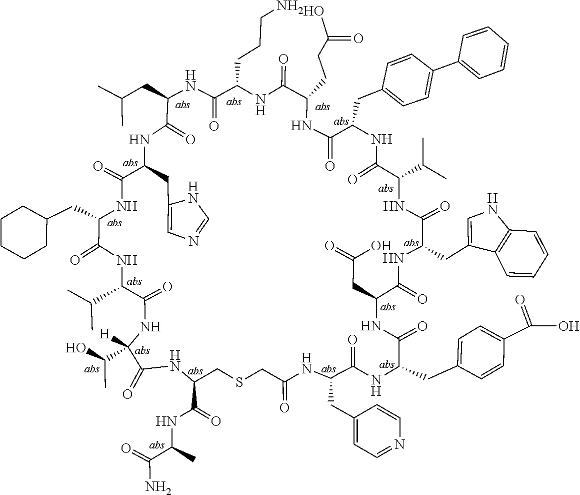 [SMILES: Cc1nc[nH]c1C(C)[C@@H]1NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1597 was prepared on a 50 μmol scale. The yield of the product was 20.4 mg, and its estimated purity by LCMS analysis was 96.3%. Analysis condition A: Retention time=1.46 min; ESI-MS(+) m/z [M+2H]²⁺: 1021.1.

### Preparation of Compound 1598

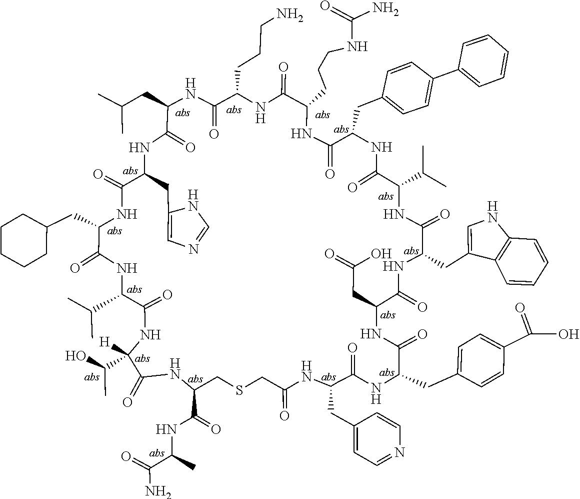 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1598 was prepared on a 50 μmol scale. The yield of the product was 28.9 mg, and its estimated purity by LCMS analysis was 97.4%. Analysis condition B: Retention time=1.52 min; ESI-MS(+) m/z [M+2H]²⁺: 1035.2.

### Preparation of Compound 1599

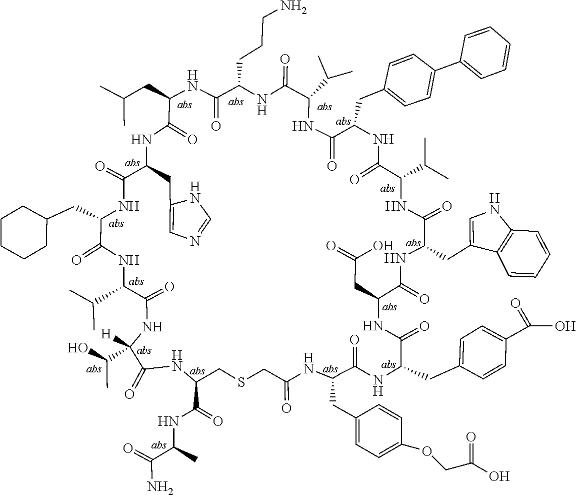 [SMILES: Cc1nc[nH]c1C(C)[C@@H]1NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1599 was prepared on a 50 μmol scale. The yield of the product was 37.1 mg, and its estimated purity by LCMS analysis was 96.5%. Analysis condition A: Retention time=1.62 min; ESI-MS(+) m/z [M+2H]²⁺: 1042.9.

### Preparation of Compound 1600

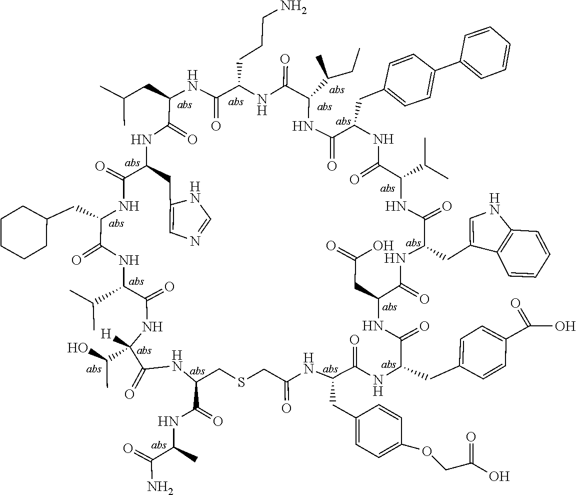 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)c2[nH]cnc2C)NC(=O)[C@@H](CC(C)C)NC(=O)[C@H](CCCN)NC1=O]

Compound 1600 was prepared on a 50 μmol scale. The yield of the product was 45.4 mg, and its estimated purity by LCMS analysis was 86%. Analysis condition B: Retention time=1.74 min; ESI-MS(+) m/z [M+2H]²⁺: 1049.2.

### Preparation of Compound 1601

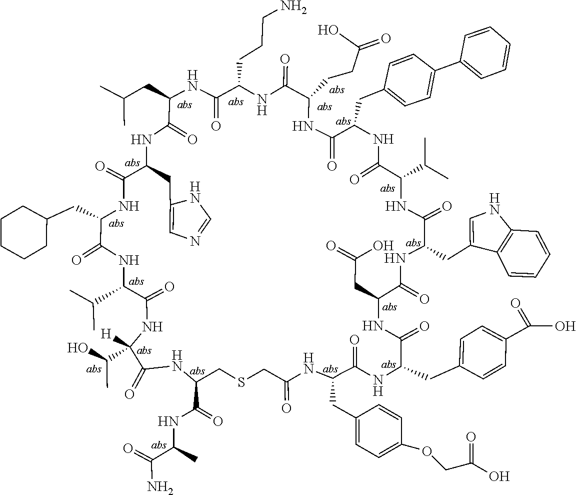 [SMILES: Cc1nc[nH]c1C(C)[C@@H]1NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1601 was prepared on a 50 μmol scale. The yield of the product was 24.6 mg, and its estimated purity by LCMS analysis was 100%. Analysis condition A: Retention time=1.45 min; ESI-MS(+) m/z [M+2H]²⁺: 1058.1.

### Preparation of Compound 1602

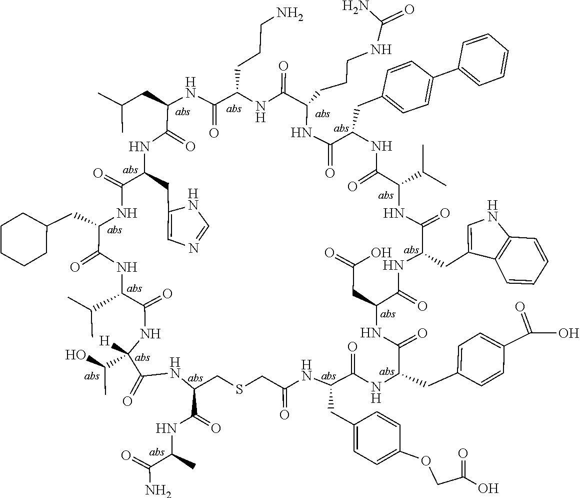 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1602 was prepared on a 50 μmol scale. The yield of the product was 19.5 mg, and its estimated purity by LCMS analysis was 98.3%. Analysis condition B: Retention time=1.65 min; ESI-MS(+) m/z [M+2H]²⁺: 1071.1.

### Preparation of Compound 1603

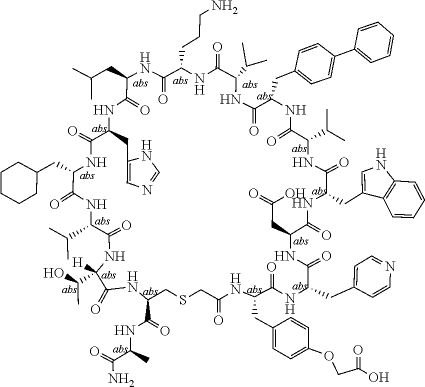 [SMILES: Cc1nc[nH]c1C(C)[C@@H]1NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1603 was prepared on a 50 μmol scale. The yield of the product was 11.6 mg, and its estimated purity by LCMS analysis was 90%. Analysis condition A: Retention time=1.82 min; ESI-MS(+) m/z [M+2H]²⁺: 1021.1.

### Preparation of Compound 1604

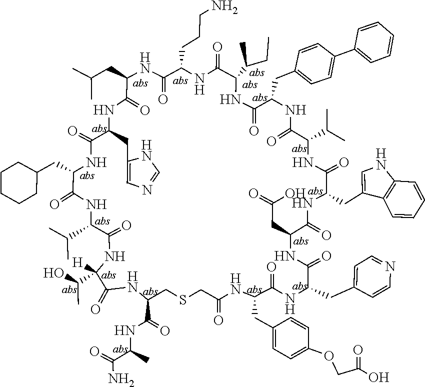 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](C(C)c2[nH]cnc2C)NC(=O)[C@@H](CC(C)C)NC(=O)[C@H](CCCN)NC1=O]

Compound 1604 was prepared on a 50 μmol scale. The yield of the product was 23.4 mg, and its estimated purity by LCMS analysis was 96%. Analysis condition A: Retention time=1.82 min; ESI-MS(+) m/z [M+3H]³⁺: 686.1.

### Preparation of Compound 1605

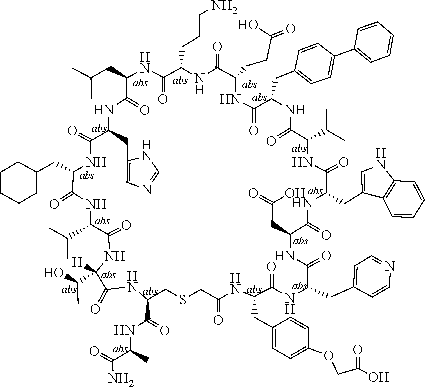 [SMILES: Cc1nc[nH]c1C(C)[C@@H]1NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1605 was prepared on a 50 μmol scale. The yield of the product was 19 mg, and its estimated purity by LCMS analysis was 95.8%. Analysis condition B: Retention time=1.55 min; ESI-MS(+) m/z [M+3H]³⁺: 691.1.

### Preparation of Compound 1606

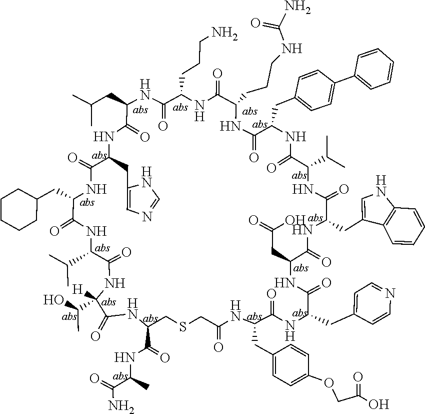 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)C[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1606 was prepared on a 50 μmol scale. The yield of the product was 21.8 mg, and its estimated purity by LCMS analysis was 96.4%. Analysis condition A: Retention time=1.58 min; ESI-MS(+) m/z [M+3H]³⁺: 700.3.

### Preparation of Compound 1607

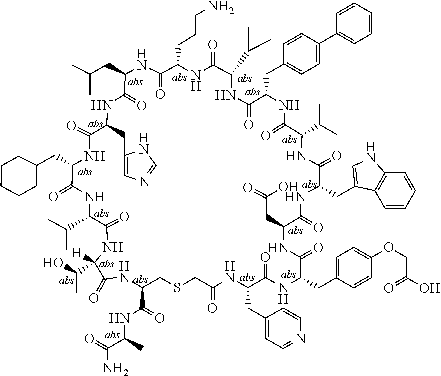 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC1=O]

Compound 1607 was prepared on a 50 μmol scale. The yield of the product was 20.7 mg, and its estimated purity by LCMS analysis was 100%. Analysis condition A: Retention time=1.73 min; ESI-MS(+) m/z [M+2H]²⁺: 1021.1.

### Preparation of Compound 1608

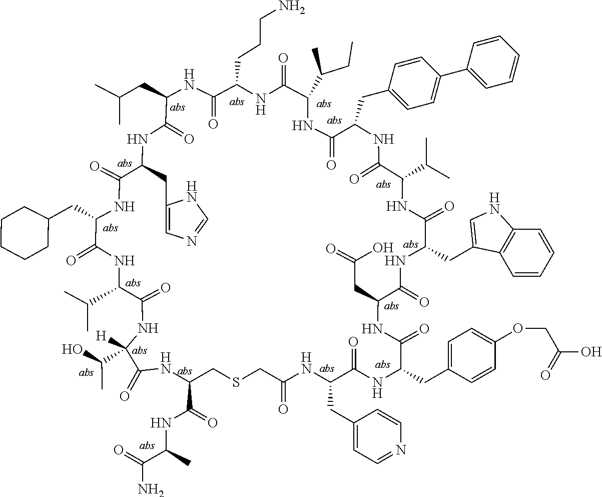 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC1=O]

Compound 1608 was prepared on a 50 μmol scale. The yield of the product was 22.9 mg, and its estimated purity by LCMS analysis was 98.5%. Analysis condition B: Retention time=1.56 min; ESI-MS(+) m/z [M+2H]²⁺: 1028.2.

### Preparation of Compound 1609

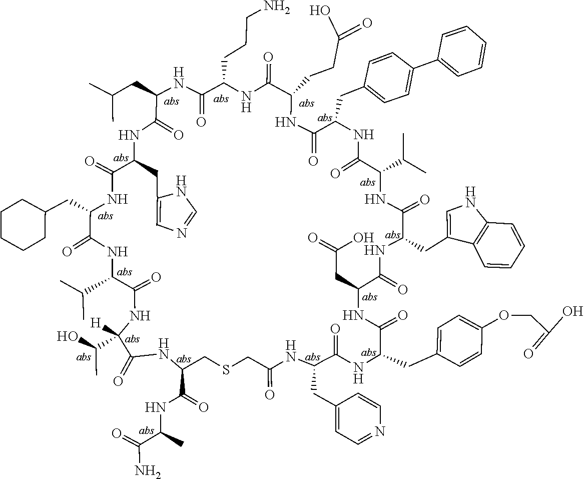 [SMILES: Cc1nc[nH]c1C(C)[C@@H]1NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1609 was prepared on a 50 μmol scale. The yield of the product was 20.6 mg, and its estimated purity by LCMS analysis was 95.2%. Analysis condition B: Retention time=1.55 min; ESI-MS(+) m/z [M+2H]²⁺: 1036.2.

### Preparation of Compound 1610

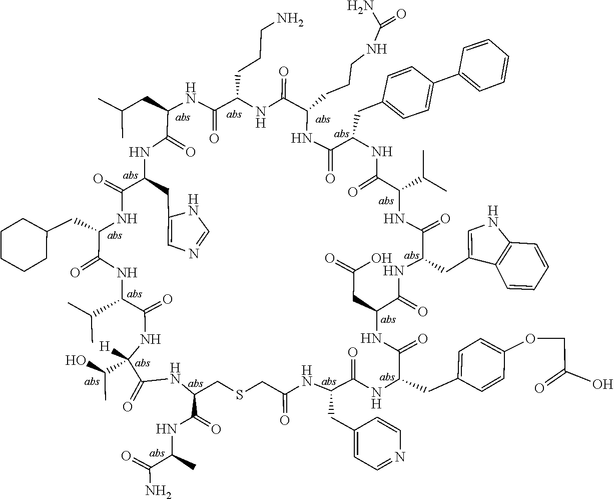 [SMILES: Cc1nc[nH]c1C(C)[C@@H]1NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)C[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1610 was prepared on a 50 μmol scale. The yield of the product was 16.1 mg, and its estimated purity by LCMS analysis was 98.7%. Analysis condition B: Retention time=1.49 min; ESI-MS(+) m/z [M+2H]²⁺: 1050.1.

### Preparation of Compound 1611

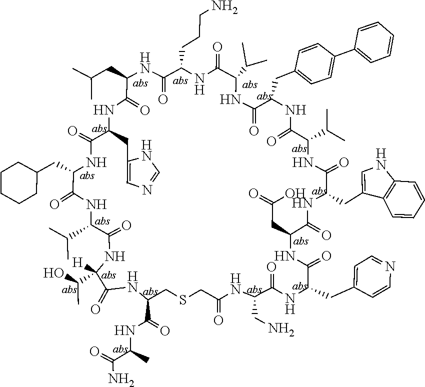 [SMILES: Cc1nc[nH]c1C(C)[C@@H]1NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)C[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC1=O]

Compound 1611 was prepared on a 50 μmol scale. The yield of the product was 42.6 mg, and its estimated purity by LCMS analysis was 93%. Analysis condition A: Retention time=1.8 min; ESI-MS(+) m/z [M+H]⁺: 1906.2.

### Preparation of Compound 1612

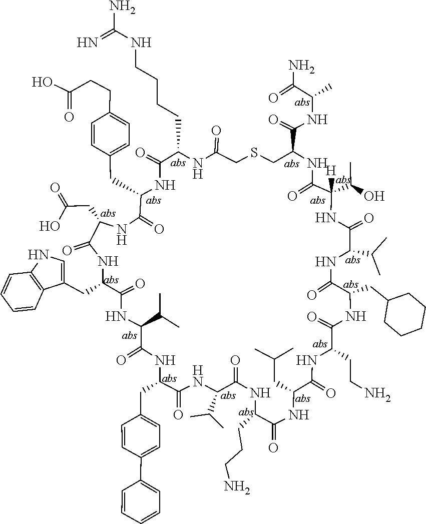 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(CCC(=O)O)cc2)NC(=O)[C@H](CCCCNC(=N)N)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1612 was prepared on a 200 μmol scale. The yield of the product was 85.1 mg and its estimated purity by LCMS analysis was 98.1%. Analysis condition A: Retention time=1.62 min; ESI-MS(+) m/z [M+2H]²⁺: 1014.1.

### Preparation of Compound 1613

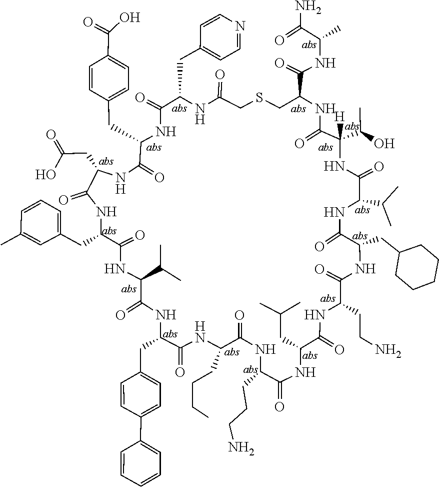 [SMILES: CCCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1613 was prepared on a 50 μmol scale. The yield of the product was 18.5 mg, and its estimated purity by LCMS analysis was 91.5%. Analysis condition A: Retention time=1.81 min; ESI-MS(+) m/z [M+H]⁺: 1963.

### Preparation of Compound 1614

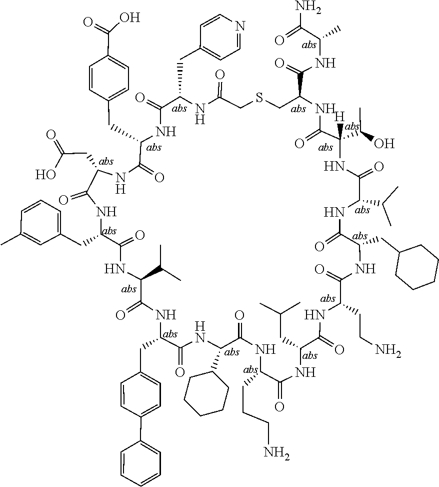 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C3CCCCC3)NC(=O)[C@H](Cc3ccc(-c4ccccc4)cc3)NC(=O)[C@H](C(C)C)NC2=O)c1]

Compound 1614 was prepared on a 50 μmol scale. The yield of the product was 11.3 mg, and its estimated purity by LCMS analysis was 93.7%. Analysis condition B: Retention time=1.6 min; ESI-MS(+) m/z [M+H]⁺: 1989.1.

### Preparation of Compound 1615

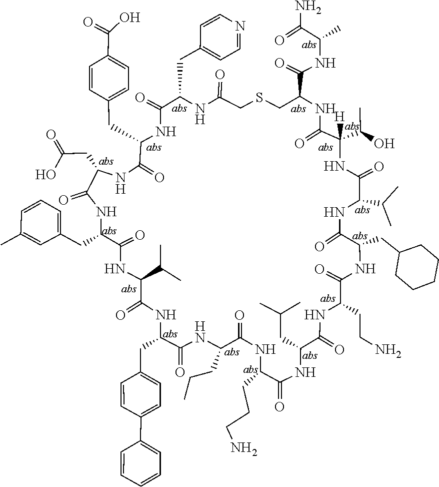 [SMILES: CCC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1615 was prepared on a 50 μmol scale. The yield of the product was 12.1 mg, and its estimated purity by LCMS analysis was 95.9%. Analysis condition A: Retention time=1.75 min; ESI-MS(+) m/z [M+H]⁺: 1949.

### Preparation of Compound 1616

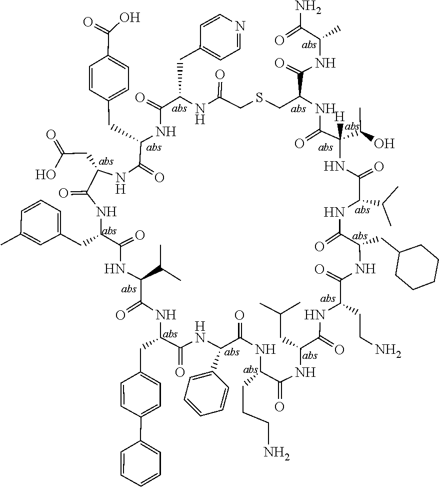 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](c3ccccc3)NC(=O)[C@H](Cc3ccc(-c4ccccc4)cc3)NC(=O)[C@H](C(C)C)NC2=O)c1]

Compound 1616 was prepared on a 50 μmol scale. The yield of the product was 12.1 mg, and its estimated purity by LCMS analysis was 89.6%. Analysis condition B: Retention time=1.5 min; ESI-MS(+) m/z [M+3H]³⁺: 662.2.

### Preparation of Compound 1617

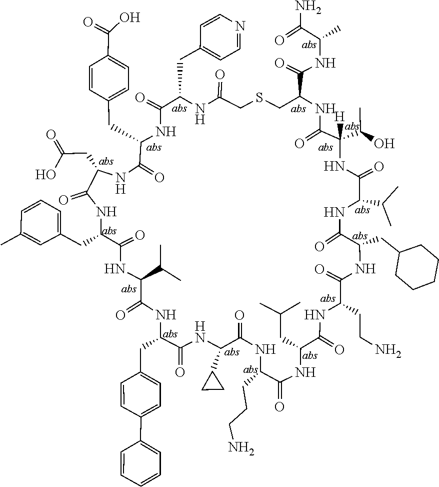 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C3CC3)NC(=O)[C@H](Cc3ccc(-c4ccccc4)cc3)NC(=O)[C@H](C(C)C)NC2=O)c1]

Compound 1617 was prepared on a 50 μmol scale. The yield of the product was 12.8 mg, and its estimated purity by LCMS analysis was 82%. Analysis condition B: Retention time=1.43 min; ESI-MS(+) m/z [M+2H]²⁺: 974.2.

### Preparation of Compound 1618

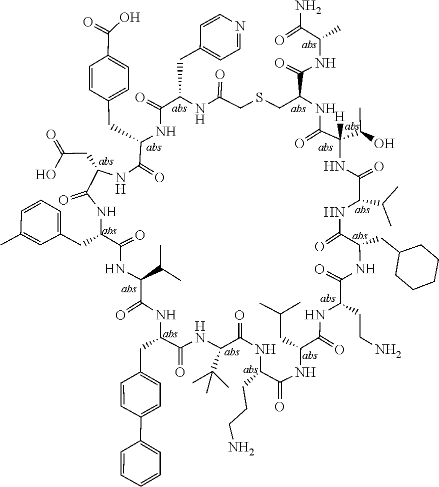 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)(C)C)NC(=O)[C@H](Cc3ccc(-c4ccccc4)cc3)NC(=O)[C@H](C(C)C)NC2=O)c1]

Compound 1618 was prepared on a 50 μmol scale. The yield of the product was 11.6 mg, and its estimated purity by LCMS analysis was 91%. Analysis condition B: Retention time=1.37, 1.39 min; ESI-MS(+) m/z [M+3H]³⁺: 655.

### Preparation of Compound 1619

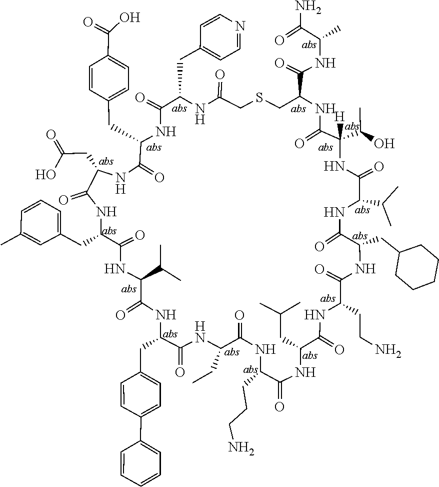 [SMILES: CC[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1619 was prepared on a 50 μmol scale. The yield of the product was 5.4 mg, and its estimated purity by LCMS analysis was 91.3%. Analysis condition B: Retention time=1.5 min; ESI-MS(+) m/z [M+H]⁺: 1935.3.

### Preparation of Compound 1620

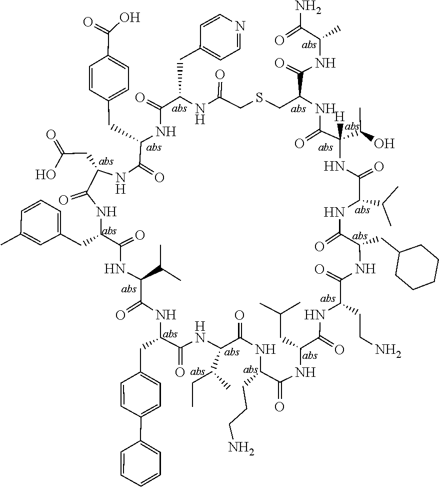 [SMILES: CC[C@@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1620 was prepared on a 50 μmol scale. The yield of the product was 10.8 mg, and its estimated purity by LCMS analysis was 95.3%. Analysis condition B: Retention time=1.43 min; ESI-MS(+) m/z [M+2H]²⁺: 982.2.

### Preparation of Compound 1621

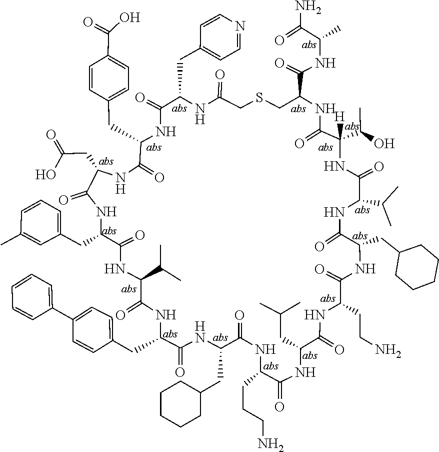 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CC3CCCCC3)NC(=O)[C@H](Cc3ccc(-c4ccccc4)cc3)NC(=O)[C@H](C(C)C)NC2=O)c1]

Compound 1621 was prepared on a 50 μmol scale. The yield of the product was 10.2 mg, and its estimated purity by LCMS analysis was 92.2%. Analysis condition B: Retention time=1.63 min; ESI-MS(+) m/z [M+2H]²⁺: 1002.2.

### Preparation of Compound 1622

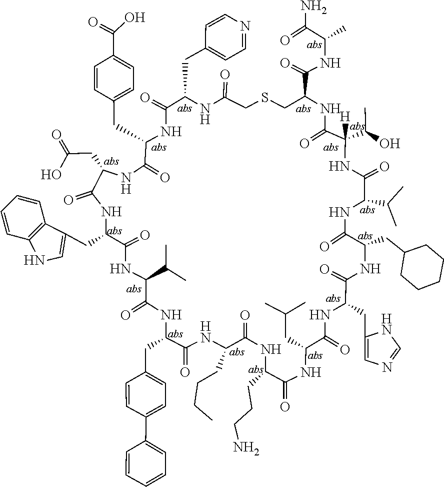 [SMILES: CCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC1=O]

Compound 1622 was prepared on a 50 μmol scale. The yield of the product was 26.1 mg, and its estimated purity by LCMS analysis was 95.7%. Analysis condition A: Retention time=1.74 min; ESI-MS(+) m/z [M+2H]²⁺: 676.2.

### Preparation of Compound 1623

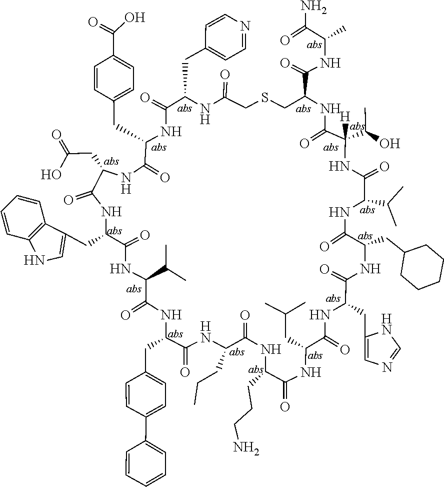 [SMILES: CCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCCN)NC1=O]

Compound 1623 was prepared on a 50 μmol scale. The yield of the product was 15.1 mg, and its estimated purity by LCMS analysis was 95.5%. Analysis condition B: Retention time=1.37 min; ESI-MS(+) m/z [M+2H]²⁺: 1006.

### Preparation of Compound 162

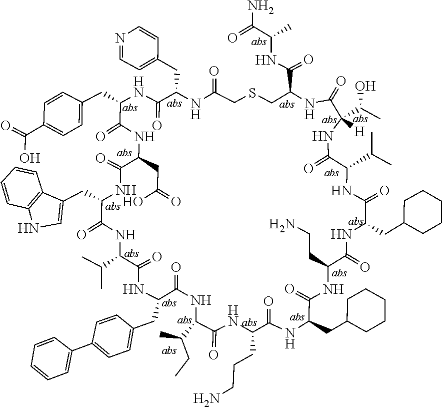 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1624 was prepared on a 50 μmol scale. The yield of the product was 17.5 mg, and its estimated purity by LCMS analysis was 88.7%. Analysis condition B: Retention time=1.55 min; ESI-MS(+) m/z [M+3H]³⁺: 677.2.

### Preparation of Compound 1625

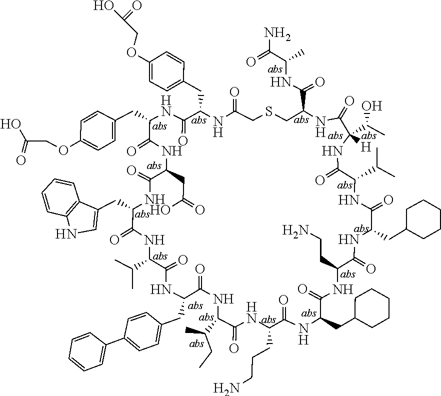 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1625 was prepared on a 50 μmol scale. The yield of the product was 13.8 mg, and its estimated purity by LCMS analysis was 86.6%. Analysis condition A: Retention time=1.93 min; ESI-MS(+) m/z [M+2H]²⁺: 1066.4.

### Preparation of Compound 1626

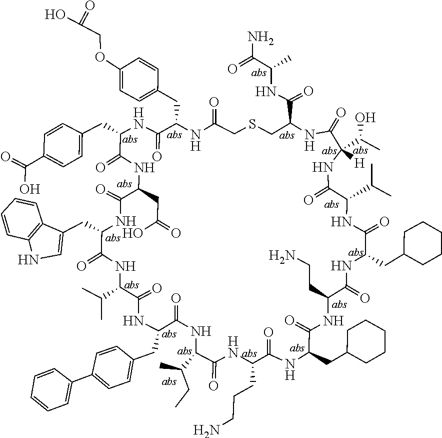 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1626 was prepared on a 50 μmol scale. The yield of the product was 2.5 mg, and its estimated purity by LCMS analysis was 95.4%. Analysis condition B: Retention time=1.78 min; ESI-MS(+) m/z [M+2H]²⁺: 1051.3.

### Preparation of Compound 1627

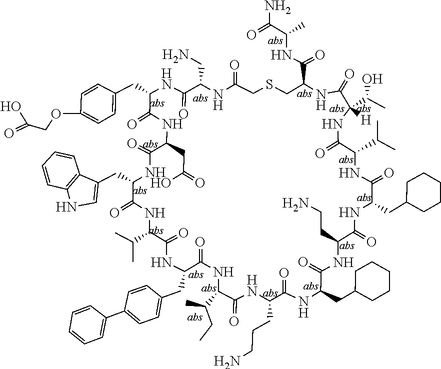 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1627 was prepared on a 50 μmol scale. The yield of the product was 11.3 mg, and its estimated purity by LCMS analysis was 94.1%. Analysis condition B: Retention time=1.5 min; ESI-MS(+) m/z [M+2H]²⁺: 999.2.

### Preparation of Compound 1628

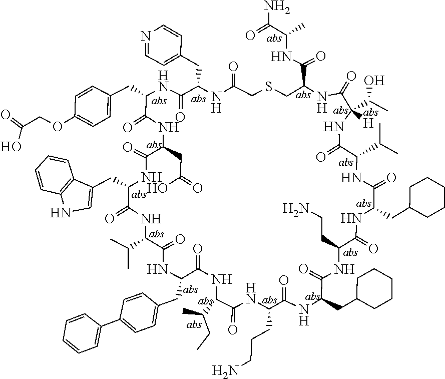 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1628 was prepared on a 50 μmol scale. The yield of the product was 4.5 mg, and its estimated purity by LCMS analysis was 95%. Analysis condition B: Retention time=1.52 min; ESI-MS(+) m/z [M+3H]³⁺: 687.

### Preparation of Compound 1629

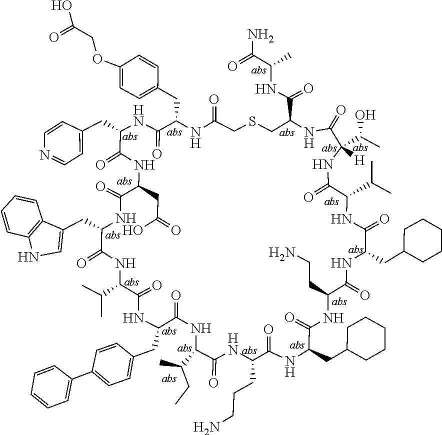 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1629 was prepared on a 50 μmol scale. The yield of the product was 1.6 mg, and its estimated purity by LCMS analysis was 98.3%. Analysis condition B: Retention time=1.62 min; ESI-MS(+) m/z [M+2H]²⁺: 1030.

### Preparation of Compound 1630

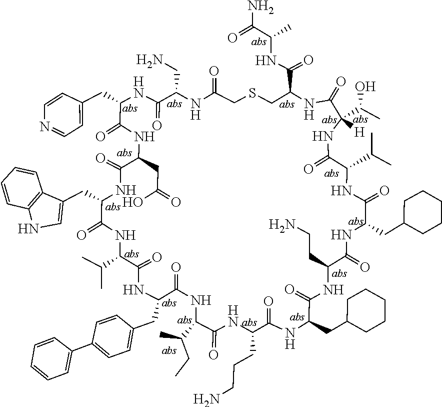 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1630 was prepared on a 50 μmol scale. The yield of the product was 4.9 mg, and its estimated purity by LCMS analysis was 95.9%. Analysis condition B: Retention time=1.5 min; ESI-MS(+) m/z [M+3H]³⁺: 642.2.

### Preparation of Compound 1631

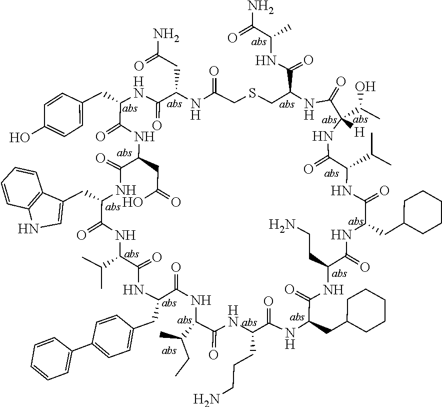 [SMILES: CC[C@@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC1=O]

Compound 1631 was prepared on a 50 μmol scale. The yield of the product was 22.5 mg, and its estimated purity by LCMS analysis was 86.1%. Analysis condition A: Retention time=2.05 min; ESI-MS(+) m/z [M+H]⁺: 1965.8.

### Preparation of Compound 1632

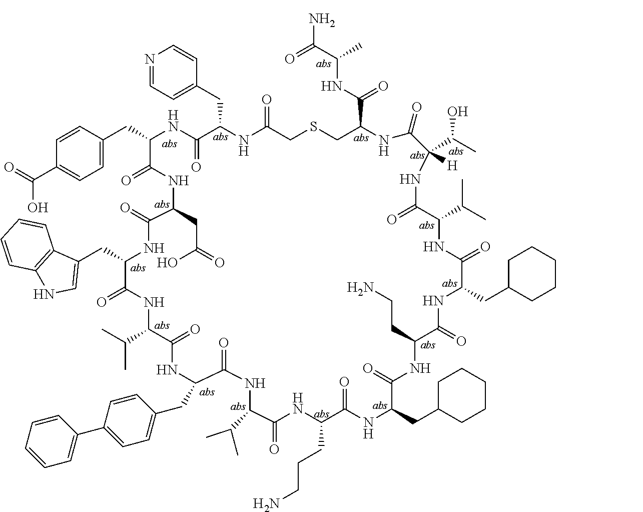 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1632 was prepared on a 50 μmol scale. The yield of the product was 7.4 mg, and its estimated purity by LCMS analysis was 100%. Analysis condition A: Retention time=1.82 min; ESI-MS(+) m/z [M+2H]²⁺: 1008.1.

### Preparation of Compound 1633

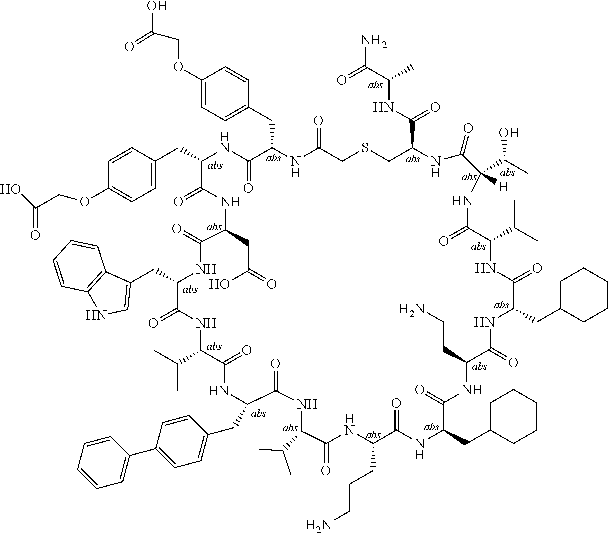 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1633 was prepared on a 50 μmol scale. The yield of the product was 15.1 mg, and its estimated purity by LCMS analysis was 92.8%. Analysis condition A: Retention time=1.86 min; ESI-MS(+) m/z [M+2H]²⁺: 1060.1.

### Preparation of Compound 1634

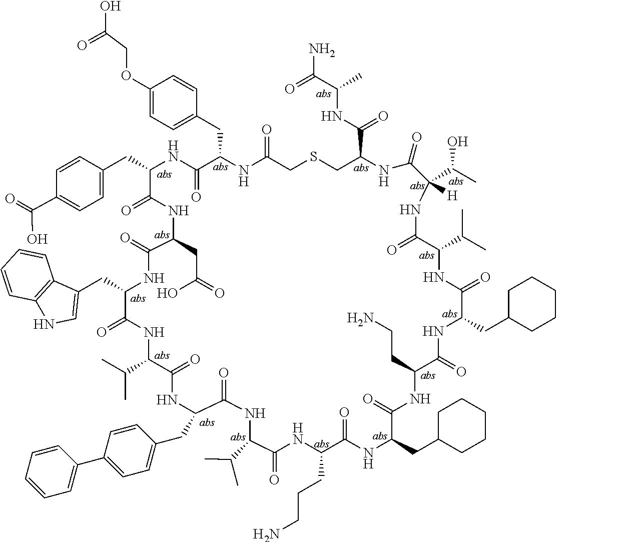 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1634 was prepared on a 50 μmol scale. The yield of the product was 21.4 mg, and its estimated purity by LCMS analysis was 88.9%. Analysis condition A: Retention time=1.72 min; ESI-MS(+) m/z [M+2H]²⁺: 1044.2.

### Preparation of Compound 1635

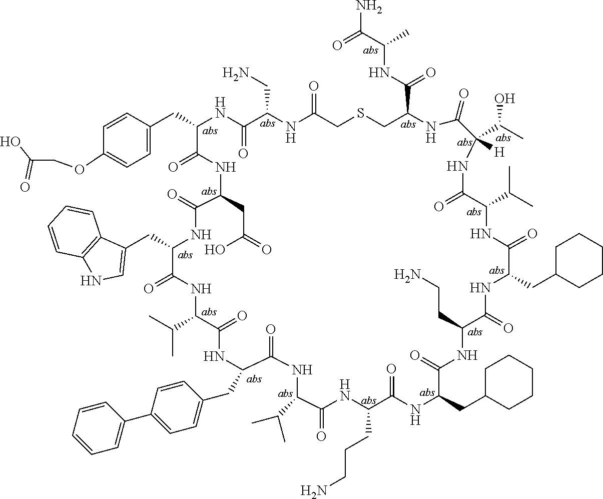 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1635 was prepared on a 50 μmol scale. The yield of the product was 12.1 mg, and its estimated purity by LCMS analysis was 85.7%. Analysis condition A: Retention time=1.84 min; ESI-MS(+) m/z [M+H]⁺: 1981.8.

### Preparation of Compound 1636

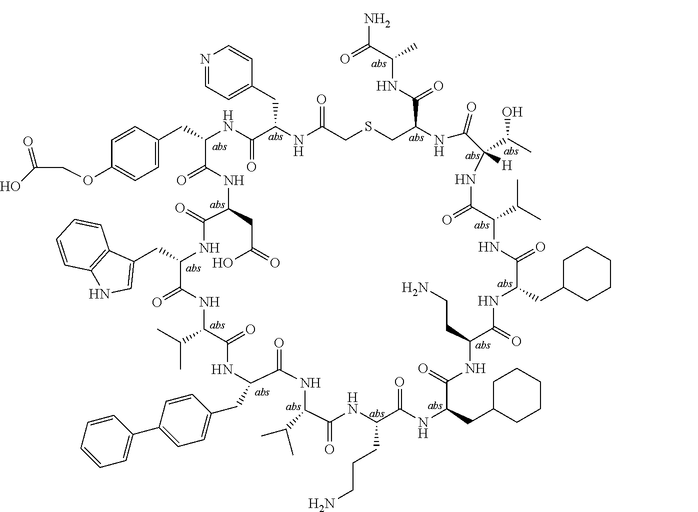 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1636 was prepared on a 50 μmol scale. The yield of the product was 5.8 mg, and its estimated purity by LCMS analysis was 94.3%. Analysis condition B: Retention time=1.5 min; ESI-MS(+) m/z [M+3]³⁺: 682.

### Preparation of Compound 1637

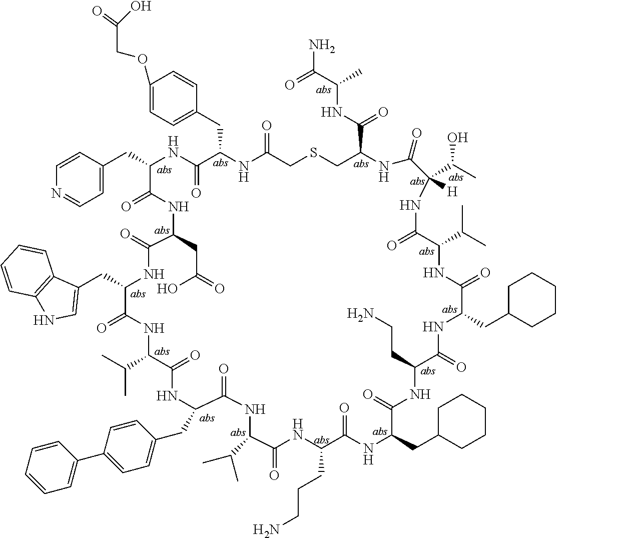 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1637 was prepared on a 50 μmol scale. The yield of the product was 8.8 mg, and its estimated purity by LCMS analysis was 86.6%. Analysis condition B: Retention time=1.5 min; ESI-MS(+) m/z [M+2H]²⁺: 1023.

### Preparation of Compound 1638

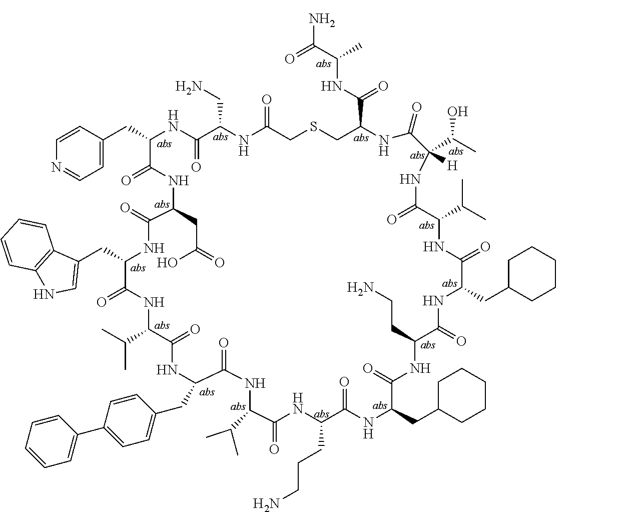 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1638 was prepared on a 50 μmol scale. The yield of the product was 16.9 mg, and its estimated purity by LCMS analysis was 98.6%. Analysis condition A: Retention time=1.61 min; ESI-MS(+) m/z [M+3H]³⁺: 637.

### Preparation of Compound 1639

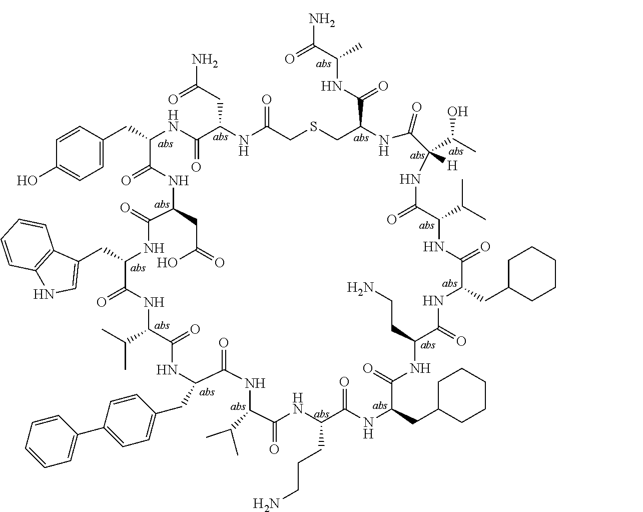 [SMILES: CC(C)[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](C(C)C)NC(=O)[C@H](Cc2ccc(-c3ccccc3)cc2)NC1=O]

Compound 1639 was prepared on a 50 μmol scale. The yield of the product was 14.5 mg, and its estimated purity by LCMS analysis was 96%. Analysis condition B: Retention time=1.6 min; ESI-MS(+) m/z [M+2H]²⁺: 977.2.

### Preparation of Compound 1640

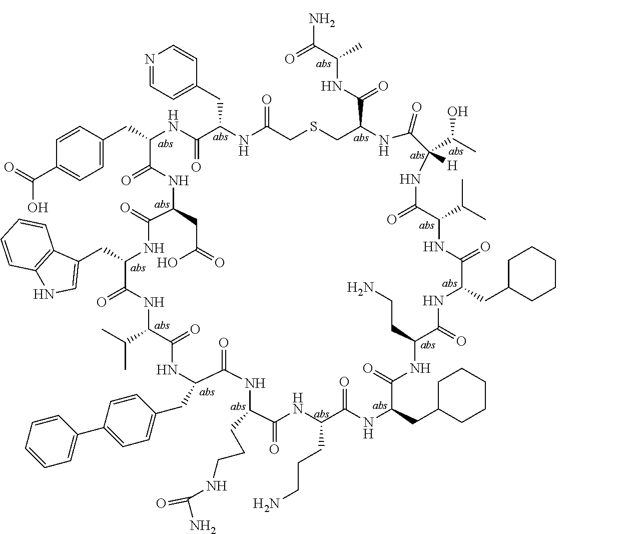 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1640 was prepared on a 50 μmol scale. The yield of the product was 21.8 mg, and its estimated purity by LCMS analysis was 85.9%. Analysis condition A: Retention time=1.57 min; ESI-MS(+) m/z [M+2H]²⁺: 1037.2.

### Preparation of Compound 1641

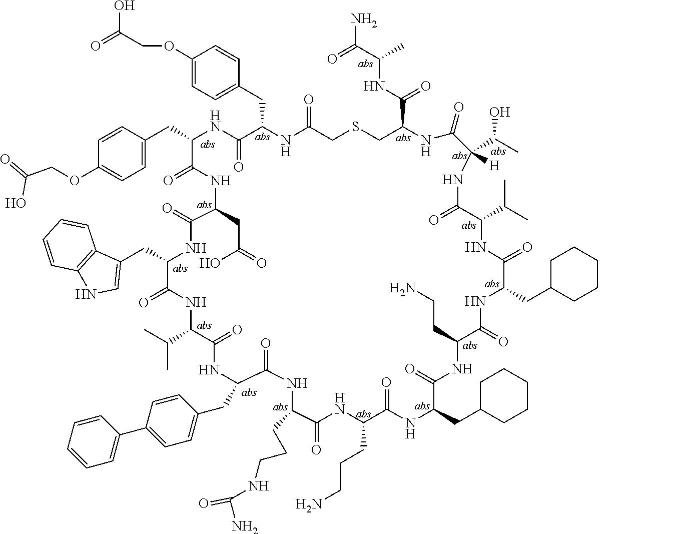 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1641 was prepared on a 50 μmol scale. The yield of the product was 16.7 mg, and its estimated purity by LCMS analysis was 91%. Analysis condition A: Retention time=1.49 min; ESI-MS(+) m/z [M+2H]²⁺: 1088.8.

### Preparation of Compound 1642

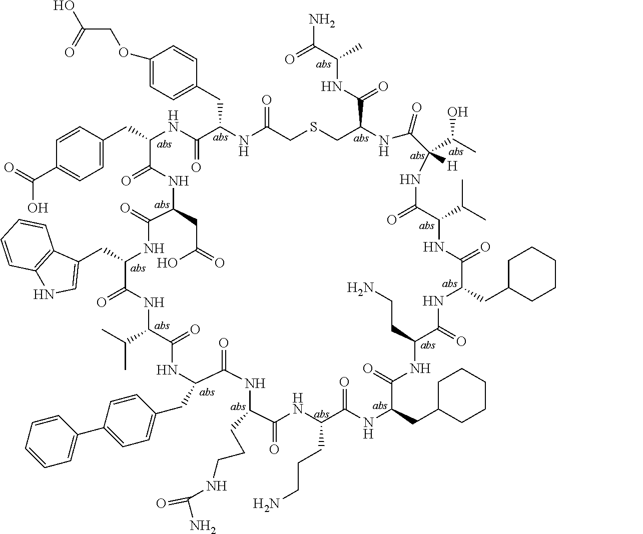 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1642 was prepared on a 50 μmol scale. The yield of the product was 8.1 mg, and its estimated purity by LCMS analysis was 96%. Analysis condition B: Retention time=1.7 min; ESI-MS(+) m/z [M+2H]²⁺: 1073.2.

### Preparation of Compound 1643

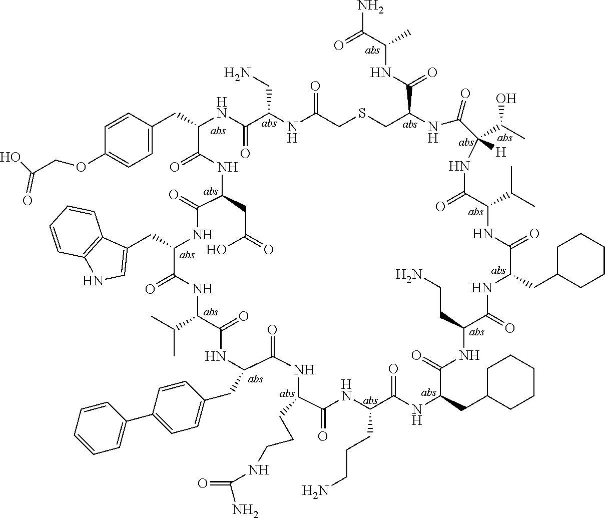 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1643 was prepared on a 50 μmol scale. The yield of the product was 14.6 mg, and its estimated purity by LCMS analysis was 94.4%. Analysis condition B: Retention time=1.7 min; ESI-MS(+) m/z [M+2H]²⁺: 1021.1.

### Preparation of Compound 1644

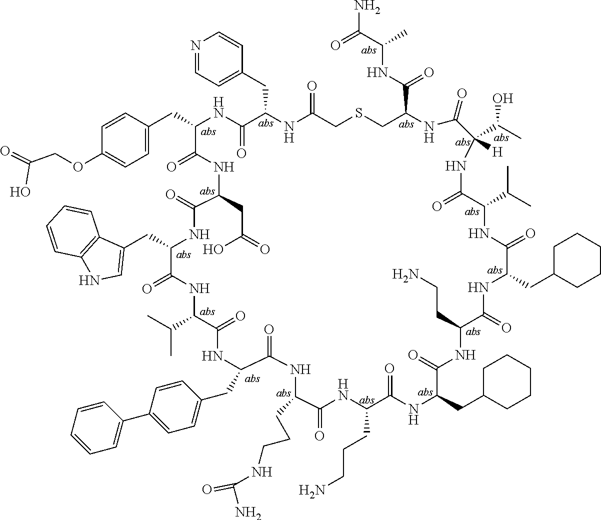 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1644 was prepared on a 50 μmol scale. The yield of the product was 10.2 mg, and its estimated purity by LCMS analysis was 91.5%. Analysis condition A: Retention time=1.54 min; ESI-MS(+) m/z [M+2H]²⁺: 1052.

### Preparation of Compound 1645

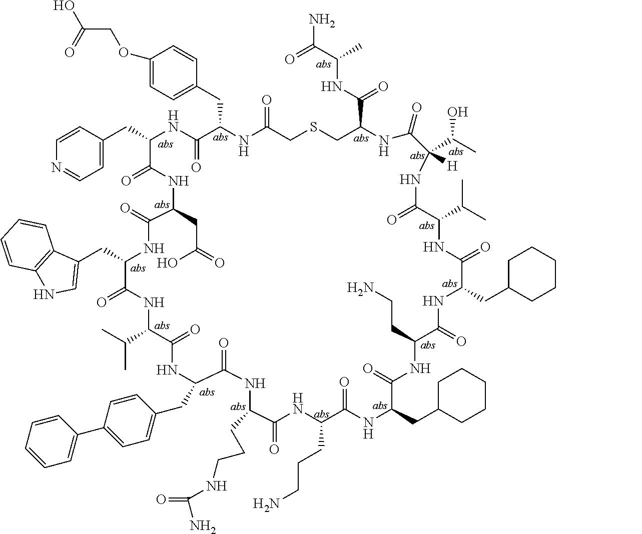 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1645 was prepared on a 50 μmol scale. The yield of the product was 15.6 mg, and its estimated purity by LCMS analysis was 90.1%. Analysis condition B: Retention time=1.51 min; ESI-MS(+) m/z [M+2H]²⁺: 1052.2.

### Preparation of Compound 1646

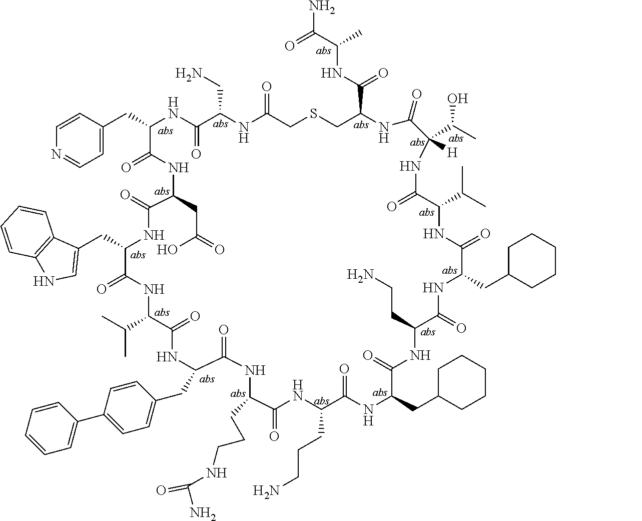 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1646 was prepared on a 50 μmol scale. The yield of the product was 12.1 mg, and its estimated purity by LCMS analysis was 88.1%. Analysis condition B: Retention time=1.59 min; ESI-MS(+) m/z [M+H]⁺: 1966.8.

### Preparation of Compound 1647

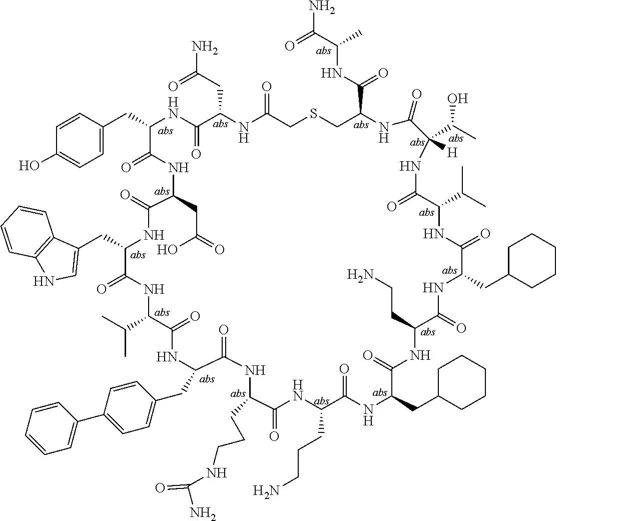 [SMILES: CC(C)[C@@H]1NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC2CCCCC2)NC(=O)[C@H](CCCN)NC(=O)[C@H](CCCNC(N)=O)NC(=O)[C@H](Cc2ccc(-c3ccccc3)cc2)NC1=O]

Compound 1647 was prepared on a 50 μmol scale. The yield of the product was 15 mg, and its estimated purity by LCMS analysis was 95.3%. Analysis condition B: Retention time=1.66 min; ESI-MS(+) m/z [M+2H]²⁺: 1006.1.

### Preparation of Compound 1648

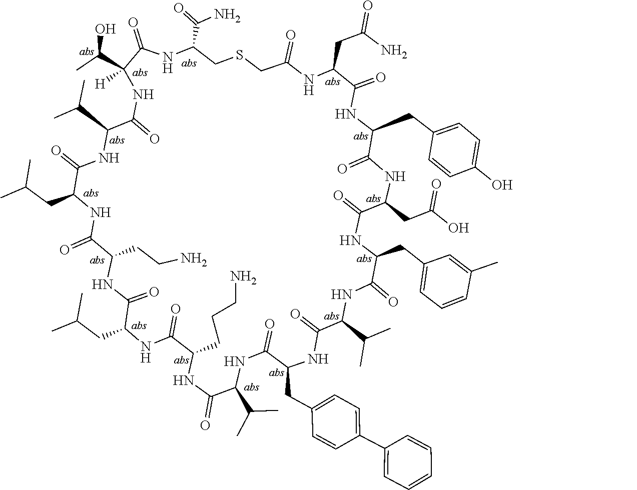 [SMILES: Cc1cccc(C[C@@H]2NC(=O)[C@H](CC(=O)O)NC(=O)[C@H](Cc3ccc(O)cc3)NC(=O)[C@H](CC(N)=O)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1648 was prepared on a 50 μmol scale. The yield of the product was 4.6 mg, and its estimated purity by LCMS analysis was 88.4%. Analysis condition B: Retention time=1.54 min; ESI-MS(+) m/z [M+2H]²⁺: 889.

### Preparation of Compound 1649

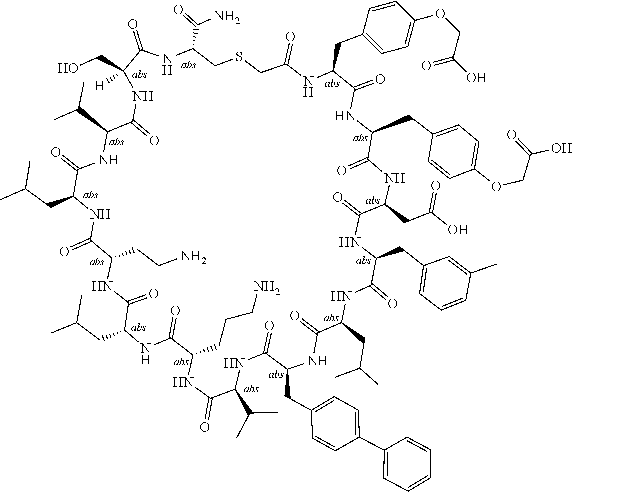 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1649 was prepared on a 50 μmol scale. The yield of the product was 2.8 mg, and its estimated purity by LCMS analysis was 88.9%. Analysis condition B: Retention time=1.64 min; ESI-MS(+) m/z [M+2H]²⁺: 971.

### Preparation of Compound 1650

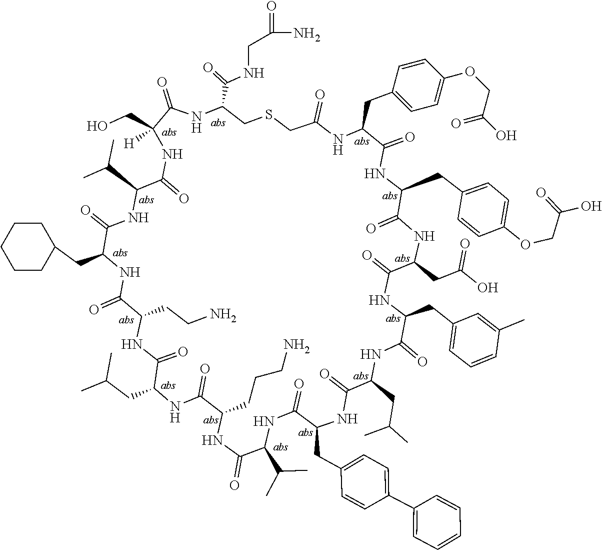 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)NCC(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](CC(C)C)NC2=O)c1]

Compound 1650 was prepared on a 50 μmol scale. The yield of the product was 5.6 mg, and its estimated purity by LCMS analysis was 89.9%. Analysis condition B: Retention time=1.68 min; ESI-MS(+) m/z [M+2H]²⁺: 1020.

### Preparation of Compound 1651

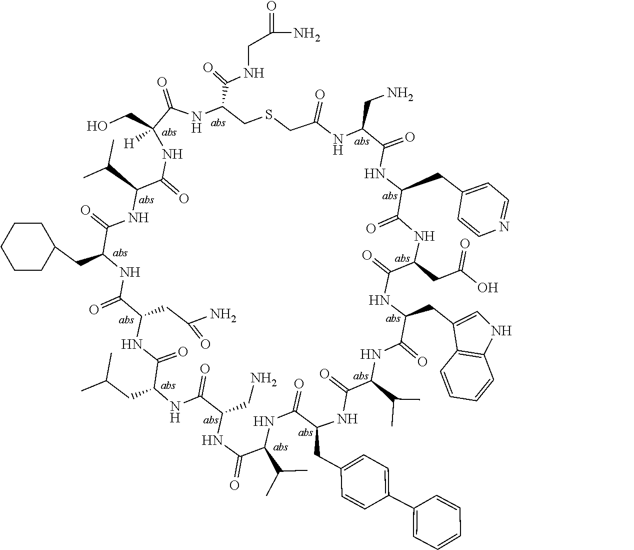 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC1=O]

Compound 1651 was prepared on a 50 μmol scale. The yield of the product was 20.9 mg, and its estimated purity by LCMS analysis was 92.2%. Analysis condition B: Retention time=1.38 min; ESI-MS(+) m/z [M+2H]²⁺: 914.2.

### Preparation of Compound 1652

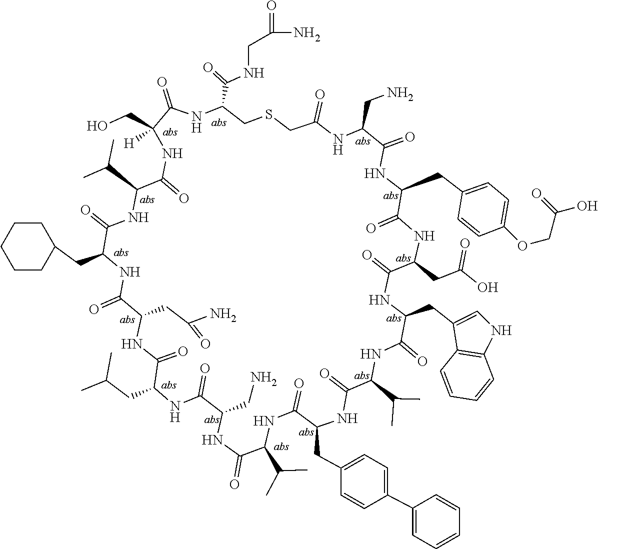 [SMILES: CC(C)C[C@H]1NC(=O)[C@H](CN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CC(N)=O)NC1=O]

Compound 1652 was prepared on a 50 μmol scale. The yield of the product was 27.6 mg, and its estimated purity by LCMS analysis was 94.1%. Analysis condition B: Retention time=1.59 min; ESI-MS(+) m/z [M+2H]²⁺: 951.

### Preparation of Compound 1653

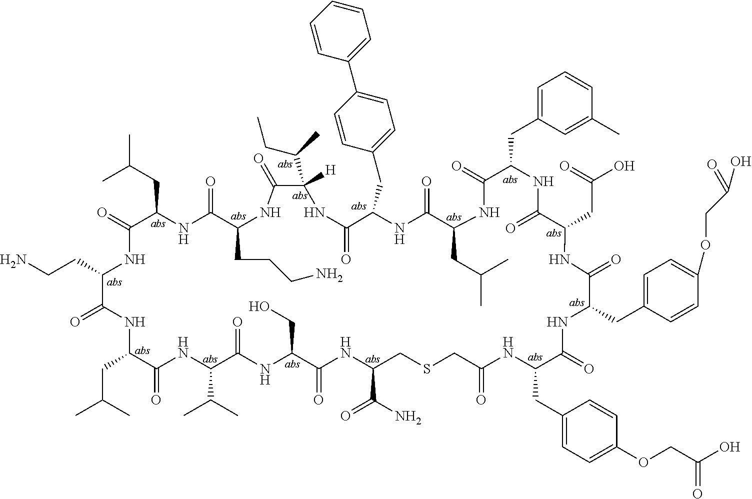 [SMILES: CC[C@@H](C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1653 was prepared on a 50 μmol scale. The yield of the product was 1.6 mg, and its estimated purity by LCMS analysis was 92.1%. Analysis condition B: Retention time=1.69 min; ESI-MS(+) m/z [M+2H]²⁺: 978.

### Preparation of Compound 1654

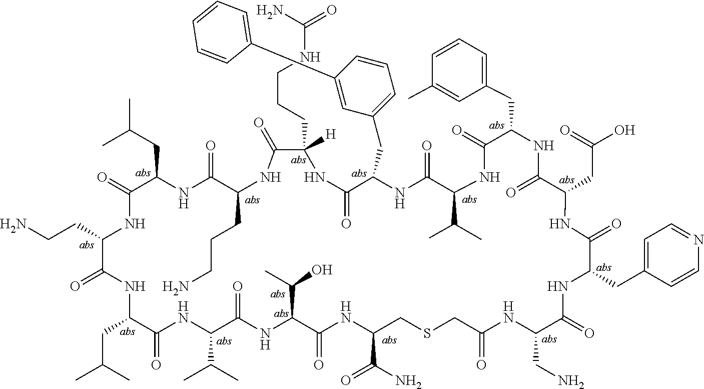 [SMILES: Cc1cccc(C[C@@H]2NC(=O)[C@H](CC(=O)O)NC(=O)[C@H](Cc3ccncc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3cccc(-c4ccccc4)c3)NC(=O)[C@H](C(C)C)NC2=O)c1]

Compound 1654 was prepared on a 50 μmol scale. The yield of the product was 6 mg, and its estimated purity by LCMS analysis was 92.9%. Analysis condition A: Retention time=1.39 min; ESI-MS(+) m/z [M+2H]²⁺: 896.4.

### Preparation of Compound 1655

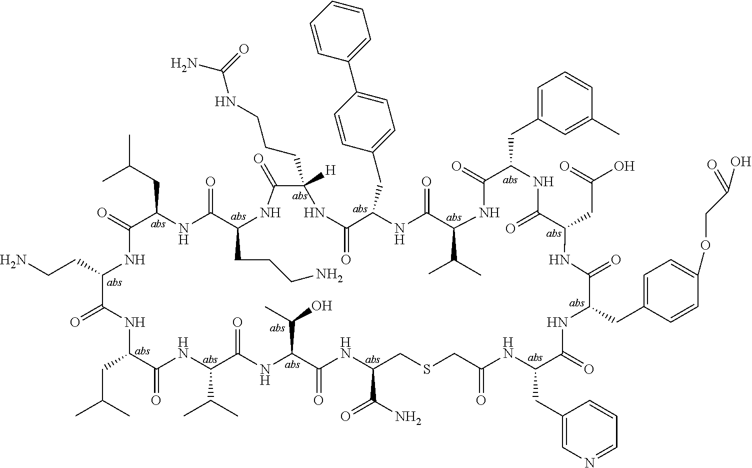 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1655 was prepared on a 50 μmol scale. The yield of the product was 2.9 mg, and its estimated purity by LCMS analysis was 98.5%. Analysis condition B: Retention time=1.46 min; ESI-MS(+) m/z [M+H]⁺: 1926.

### Preparation of Compound 1656

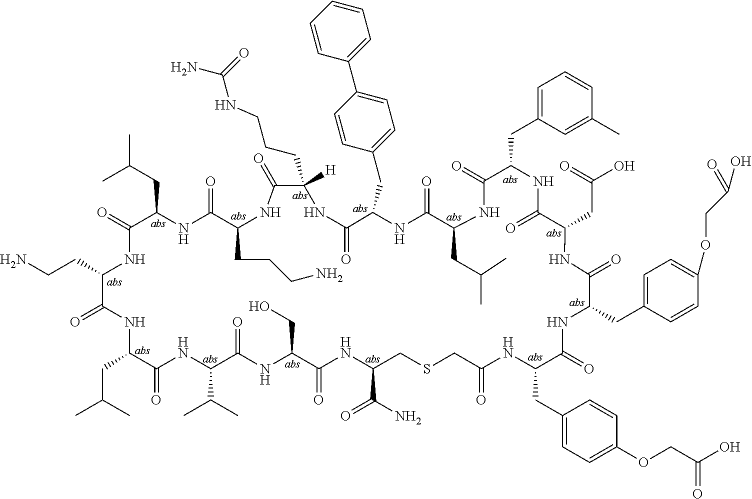 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(N)=O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](CC(C)C)NC2=O)c1]

Compound 1656 was prepared on a 50 μmol scale. The yield of the product was 3.7 mg, and its estimated purity by LCMS analysis was 93.5%. Analysis condition B: Retention time=1.59 min; ESI-MS(+) m/z [M+3H]³⁺: 667.2.

### Preparation of Compound 1657

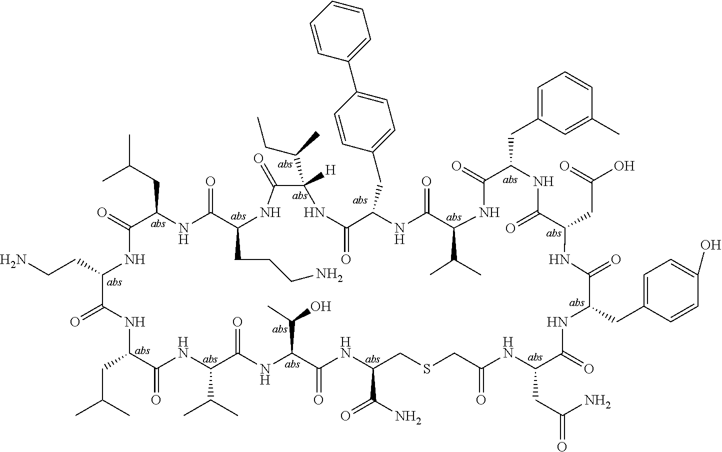 [SMILES: CC[C@@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(O)cc2)NC(=O)[C@H](CC(N)=O)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 1657 was prepared on a 50 μmol scale. The yield of the product was 7.3 mg, and its estimated purity by LCMS analysis was 85.3%. Analysis condition A: Retention time=1.68 min; ESI-MS(+) m/z [M+2H]²⁺: 896.

### Preparation of Compound 1658

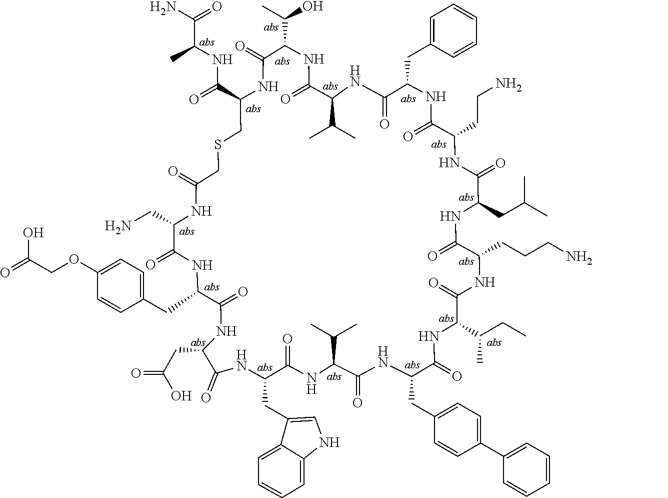 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC(=O)[C@@H](CC(C)C)NC(=O)[C@H](CCCN)NC1=O]

Compound 1658 was prepared on a 50 μmol scale. The yield of the product was 29 mg, and its estimated purity by LCMS analysis was 89.8%. Analysis condition A: Retention time=1.46 min; ESI-MS(+) m/z [M+2H]²⁺: 976.

### Preparation of Compound 1659

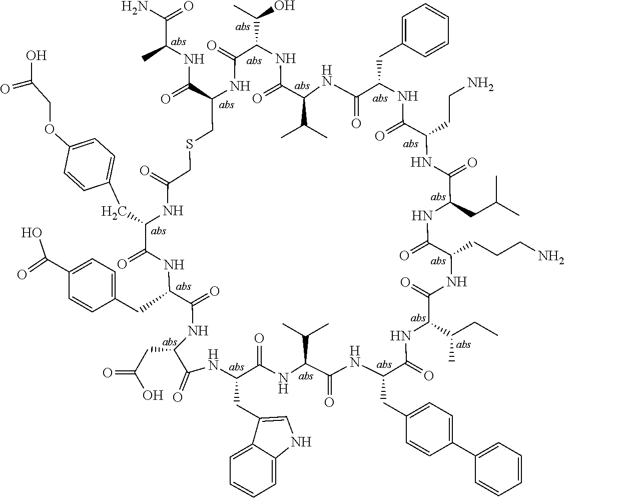 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC(=O)[C@@H](CC(C)C)NC(=O)[C@H](CCCN)NC1=O]

Compound 1659 was prepared on a 50 μmol scale. The yield of the product was 25.2 mg, and its estimated purity by LCMS analysis was 85.2%. Analysis condition A: Retention time=1.6 min; ESI-MS(+) m/z [M+2H]²⁺: 1028.3.

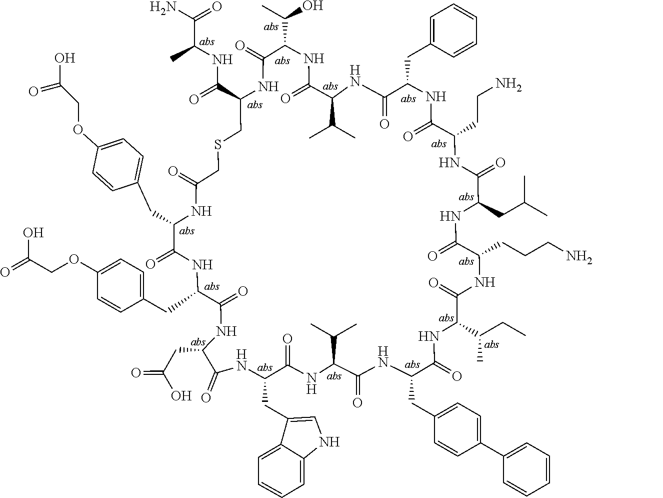 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC(=O)[C@@H](CC(C)C)NC(=O)[C@H](CCCN)NC1=O]

Compound 1660 was prepared on a 50 μmol scale. The yield of the product was 26 mg, and its estimated purity by LCMS analysis was 91%. Analysis condition A: Retention time=1.76 min; ESI-MS(+) m/z [M+2H]²⁺: 1043.2.

### Preparation of Compound 1661

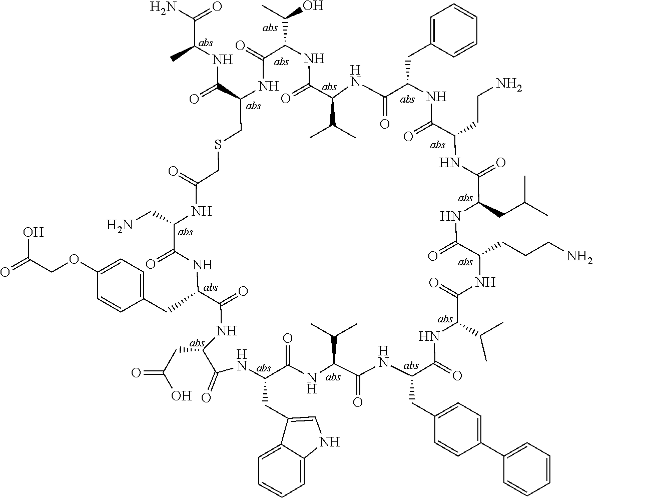 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1661 was prepared on a 50 μmol scale. The yield of the product was 21 mg, and its estimated purity by LCMS analysis was 91.1%. Analysis condition B: Retention time=1.43 min; ESI-MS(+) m/z [M+2H]²⁺: 969.2.

### Preparation of Compound 1662

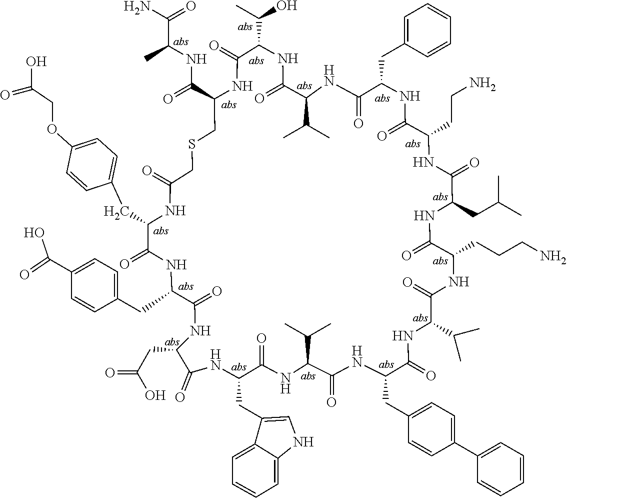 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1662 was prepared on a 50 μmol scale. The yield of the product was 21.5 mg, and its estimated purity by LCMS analysis was 94.5%. Analysis condition A: Retention time=1.72 min; ESI-MS(+) m/z [M+2H]²⁺: 1021.

### Preparation of Compound 1663

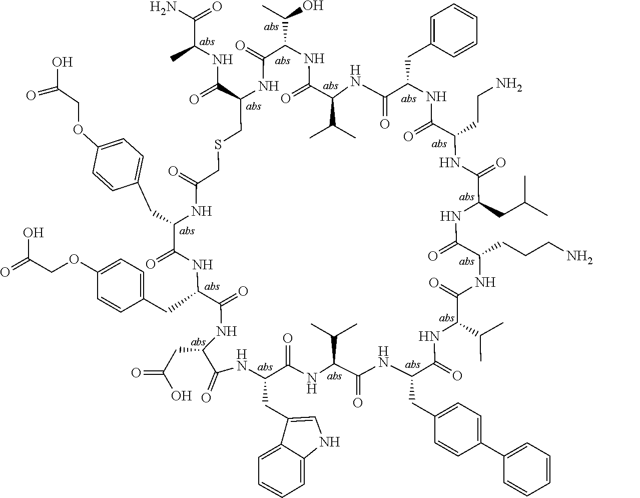 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1663 was prepared on a 50 μmol scale. The yield of the product was 18.3 mg, and its estimated purity by LCMS analysis was 94.7%. Analysis condition B: Retention time=1.61 min; ESI-MS(+) m/z [M+2H]²⁺: 1036.3.

### Preparation of Compound 1664

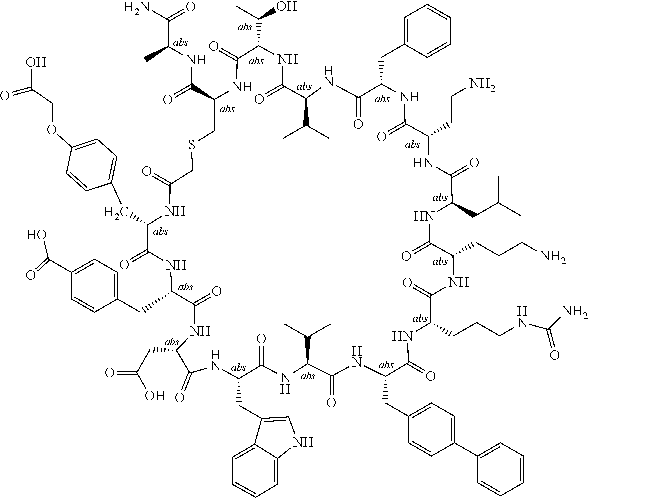 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1664 was prepared on a 50 μmol scale. The yield of the product was 12.5 mg, and its estimated purity by LCMS analysis was 87.7%. Analysis condition B: Retention time=1.48, 1.51 min; ESI-MS(+) m/z [M+2H]²⁺: 1050.33, 1050.33.

### Preparation of Compound 1665

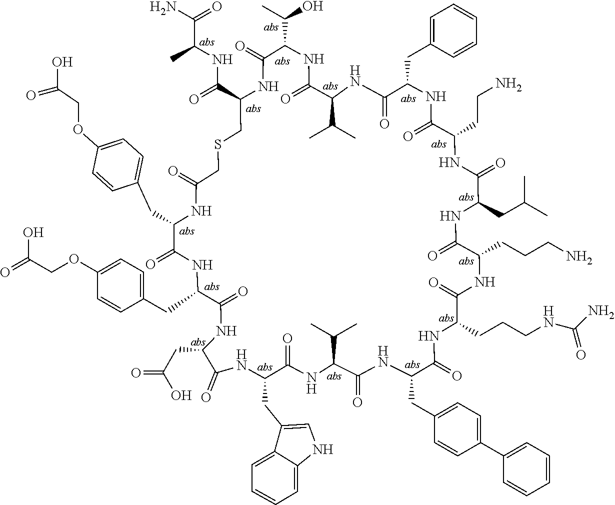 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1665 was prepared on a 50 μmol scale. The yield of the product was 21.4 mg, and its estimated purity by LCMS analysis was 85.6%. Analysis condition A: Retention time=1.52 min; ESI-MS(+) m/z [M+2H]²⁺: 1065.

### Preparation of Compound 1666

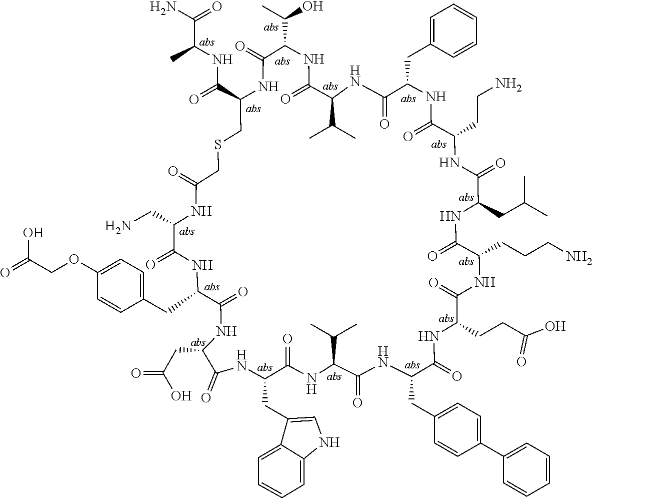 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1666 was prepared on a 50 μmol scale. The yield of the product was 19.7 mg, and its estimated purity by LCMS analysis was 100%. Analysis condition A: Retention time=1.47 min; ESI-MS(+) m/z [M+H]⁺: 1966.2.

### Preparation of Compound 1667

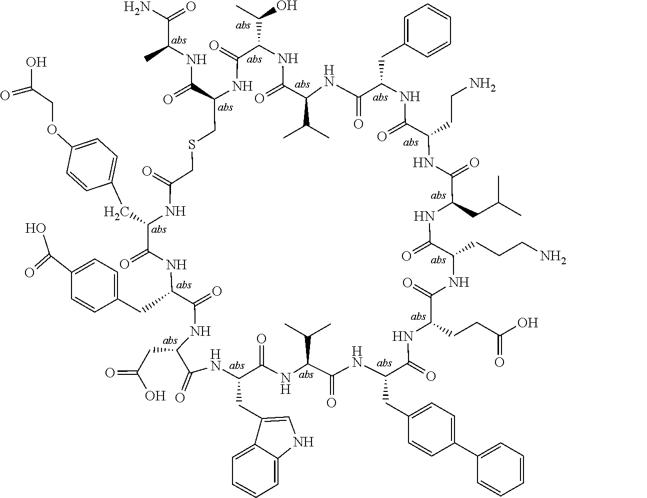 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1667 was prepared on a 50 μmol scale. The yield of the product was 16.9 mg, and its estimated purity by LCMS analysis was 92.7%. Analysis condition B: Retention time=1.54 min; ESI-MS(+) m/z [M+2H]²⁺: 1036.

### Preparation of Compound 1668

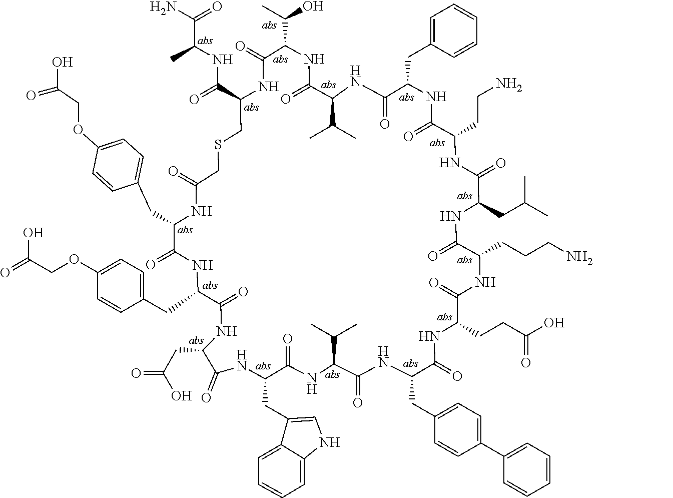 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccccc2)NC(=O)[C@H](CCN)NC1=O]

Compound 1668 was prepared on a 50 μmol scale. The yield of the product was 21.5 mg, and its estimated purity by LCMS analysis was 97.4%. Analysis condition A: Retention time=1.33 min; ESI-MS(+) m/z [M+2H]²⁺: 1051.2.

### Preparation of Compound 1669

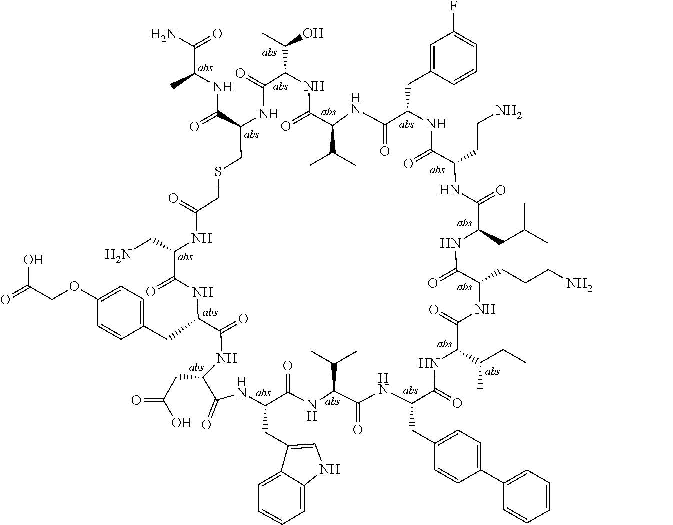 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC(=O)[C@@H](CC(C)C)NC(=O)[C@H](CCCN)NC1=O]

Compound 1669 was prepared on a 50 μmol scale. The yield of the product was 17.6 mg, and its estimated purity by LCMS analysis was 98.3%. Analysis condition B: Retention time=1.38 min; ESI-MS(+) m/z [M+H]⁺: 1968.

### Preparation of Compound 1670

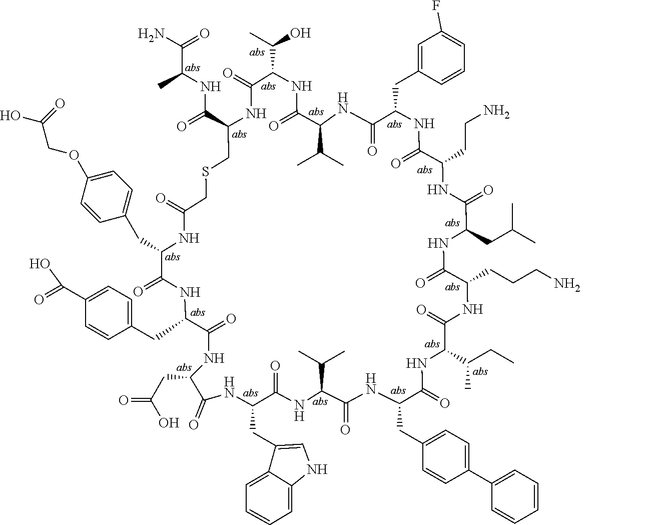 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC(=O)[C@@H](CC(C)C)NC(=O)[C@H](CCCN)NC1=O]

Compound 1670 was prepared on a 50 μmol scale. The yield of the product was 21.9 mg, and its estimated purity by LCMS analysis was 95.4%. Analysis condition B: Retention time=1.69 min; ESI-MS(+) m/z [M+2H]²⁺: 1037.3.

### Preparation of Compound 1671

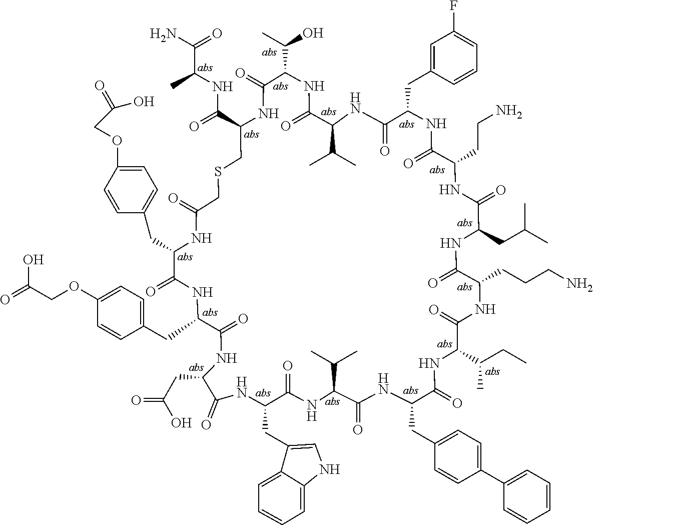 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC(=O)[C@@H](CC(C)C)NC(=O)[C@H](CCCN)NC1=O]

Compound 1671 was prepared on a 50 μmol scale. The yield of the product was 19.5 mg, and its estimated purity by LCMS analysis was 87.5%. Analysis condition A: Retention time=1.69 min; ESI-MS(+) m/z [M+2H]²⁺: 1052.

### Preparation of Compound 1672

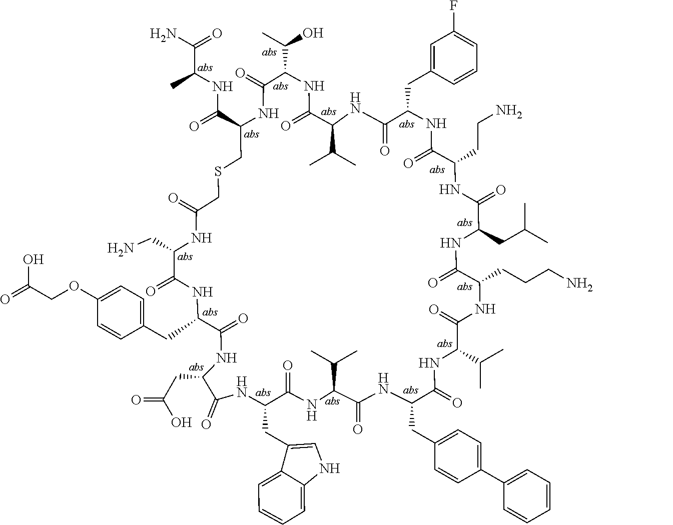 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1672 was prepared on a 50 μmol scale. The yield of the product was 16.8 mg, and its estimated purity by LCMS analysis was 94.9%. Analysis condition B: Retention time=1.43 min; ESI-MS(+) m/z [M+H]⁺: 1954.2.

### Preparation of Compound 1673

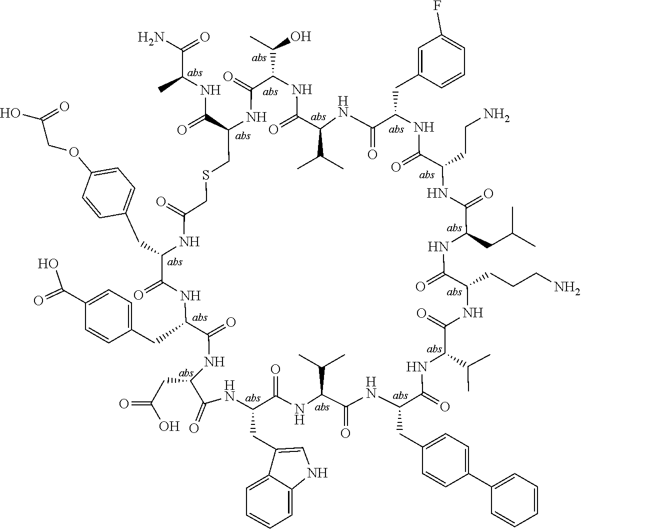 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1673 was prepared on a 50 μmol scale. The yield of the product was 12.2 mg, and its estimated purity by LCMS analysis was 86.8%. Analysis condition B: Retention time=1.67 min; ESI-MS(+) m/z [M+2H]²⁺: 1030.1.

### Preparation of Compound 1674

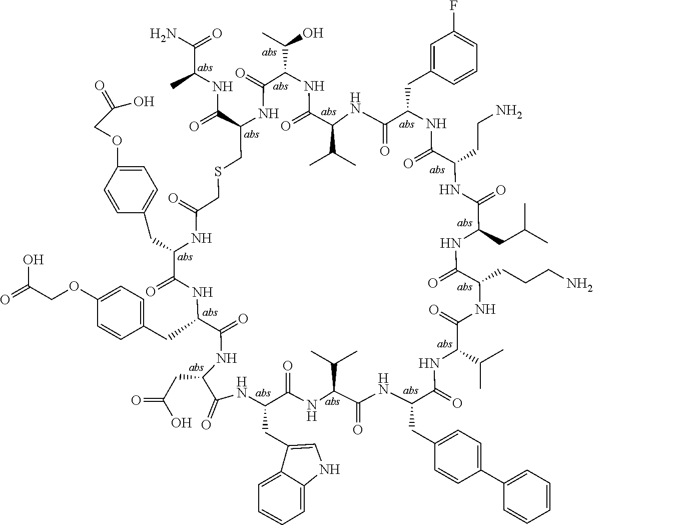 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1674 was prepared on a 50 μmol scale. The yield of the product was 27.6 mg, and its estimated purity by LCMS analysis was 97.2%. Analysis condition B: Retention time=1.54 min; ESI-MS(+) m/z [M+2H]²⁺: 1045.1.

### Preparation of Compound 1675

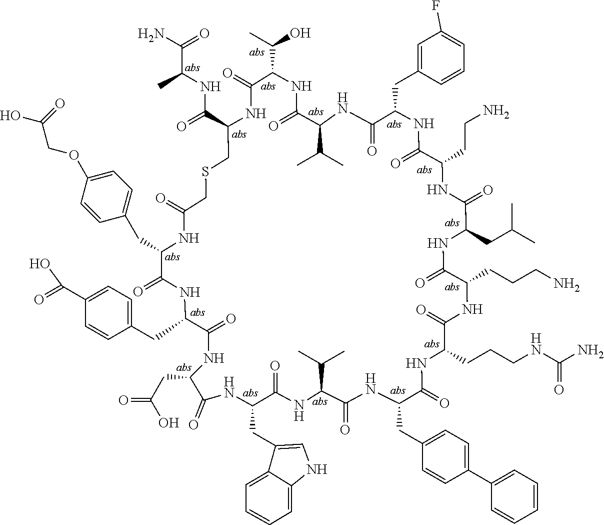 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1675 was prepared on a 50 μmol scale. The yield of the product was 15.8 mg, and its estimated purity by LCMS analysis was 96.6%. Analysis condition B: Retention time=1.53 min; ESI-MS(+) m/z [M+2H]²⁺: 1059.

### Preparation of Compound 1676

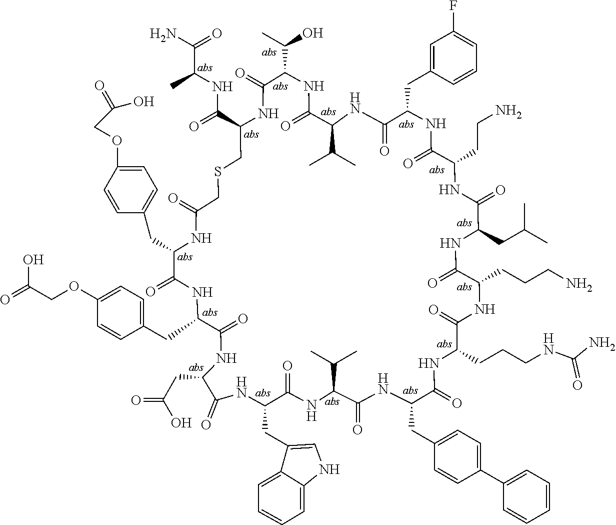 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1676 was prepared on a 50 μmol scale. The yield of the product was 14.9 mg, and its estimated purity by LCMS analysis was 98.2%. Analysis condition B: Retention time=1.53 min; ESI-MS(+) m/z [M+2H]²⁺: 1074.2.

### Preparation of Compound 1677

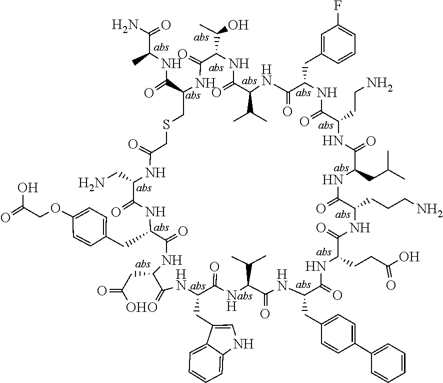 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2cccc(F)c2)NC(=O)[C@H](CCN)NC1=O]

Compound 1677 was prepared on a 50 μmol scale. The yield of the product was 14.6 mg, and its estimated purity by LCMS analysis was 87.7%. Analysis condition B: Retention time=1.43 min; ESI-MS(+) m/z [M+H]⁺: 1983.7.

### Preparation of Compound 1678

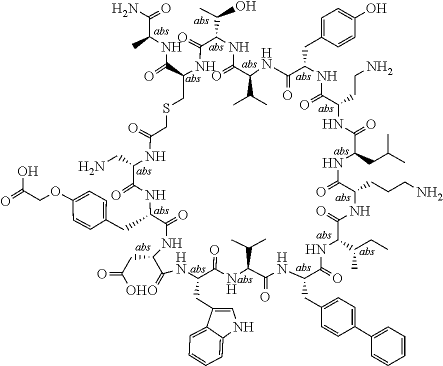 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC(=O)[C@@H](CC(C)C)NC(=O)[C@H](CCCN)NC1=O]

Compound 1678 was prepared on a 50 μmol scale. The yield of the product was 19.7 mg, and its estimated purity by LCMS analysis was 100%. Analysis condition A: Retention time=1.55 min; ESI-MS(+) m/z [M+H]⁺: 1966.1.

### Preparation of Compound 1679

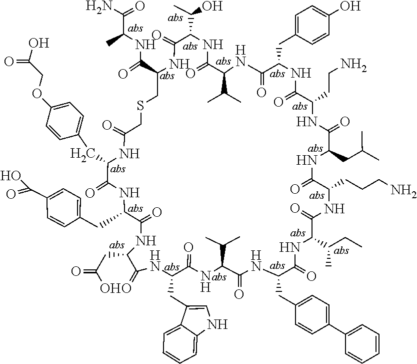 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC(=O)[C@@H](CC(C)C)NC(=O)[C@H](CCCN)NC1=O]

Compound 1679 was prepared on a 50 μmol scale. The yield of the product was 19.3 mg, and its estimated purity by LCMS analysis was 88.8%. Analysis condition A: Retention time=1.59 min; ESI-MS(+) m/z [M+2H]²⁺: 1036.2.

### Preparation of Compound 1680

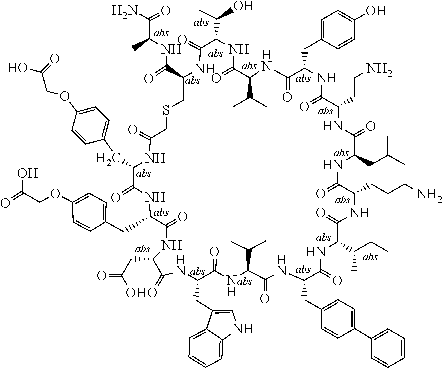 [SMILES: CC[C@H](C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC(=O)[C@@H](CC(C)C)NC(=O)[C@H](CCCN)NC1=O]

Compound 1680 was prepared on a 50 μmol scale. The yield of the product was 22.7 mg, and its estimated purity by LCMS analysis was 96.1%. Analysis condition A: Retention time=1.62 min; ESI-MS(+) m/z [M+2H]²⁺: 1051.1.

### Preparation of Compound 1681

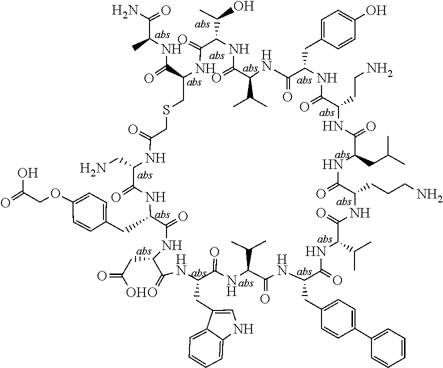 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1681 was prepared on a 50 μmol scale. The yield of the product was 23.5 mg, and its estimated purity by LCMS analysis was 93.1%. Analysis condition A: Retention time=1.48 min; ESI-MS(+) m/z [M+H]⁺: 1952.2.

### Preparation of Compound 1682

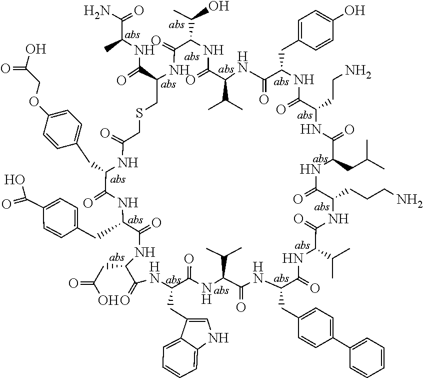 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1682 was prepared on a 50 μmol scale. The yield of the product was 21.7 mg, and its estimated purity by LCMS analysis was 100%. Analysis condition A: Retention time=1.52 min; ESI-MS(+) m/z [M+2H]²⁺: 1029.2.

### Preparation of Compound 1683

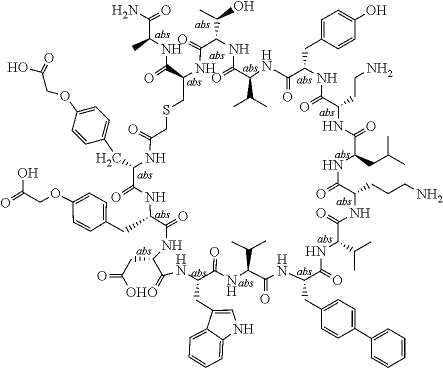 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1683 was prepared on a 50 μmol scale. The yield of the product was 21 mg, and its estimated purity by LCMS analysis was 95.5%. Analysis condition A: Retention time=1.6 min; ESI-MS(+) m/z [M+2H]²⁺: 1044.

### Preparation of Compound 1684

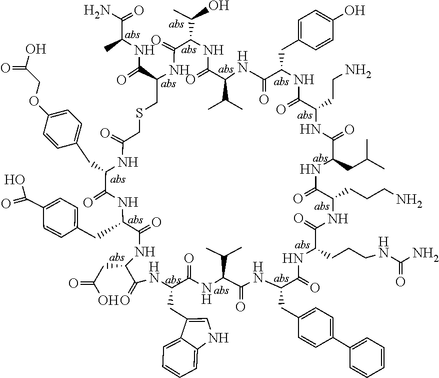 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1684 was prepared on a 50 μmol scale. The yield of the product was 17.5 mg, and its estimated purity by LCMS analysis was 92.6%. Analysis condition B: Retention time=1.4 min; ESI-MS(+) m/z [M+2H]²⁺: 1058.1.

### Preparation of Compound 1685

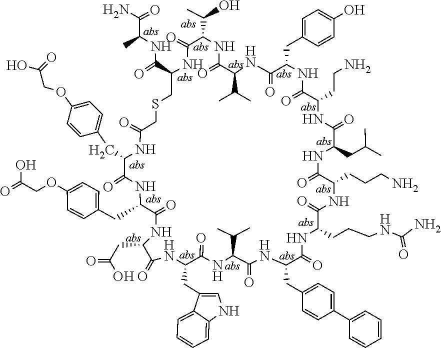 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1685 was prepared on a 50 μmol scale. The yield of the product was 10.9 mg, and its estimated purity by LCMS analysis was 97.7%. Analysis condition B: Retention time=1.48 min; ESI-MS(+) m/z [M+2H]²⁺: 1073.8.

### Preparation of Compound 1686

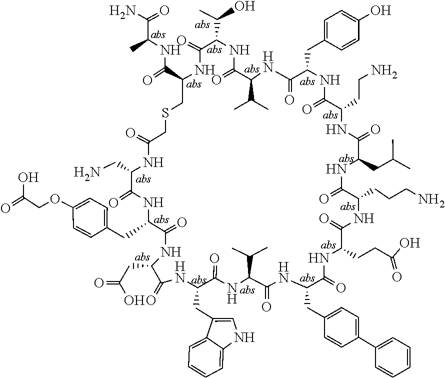 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1686 was prepared on a 50 μmol scale. The yield of the product was 6.4 mg, and its estimated purity by LCMS analysis was 91.6%. Analysis condition B: Retention time=1.43 min; ESI-MS(+) m/z [M+H]⁺: 1982.6.

### Preparation of Compound 1687

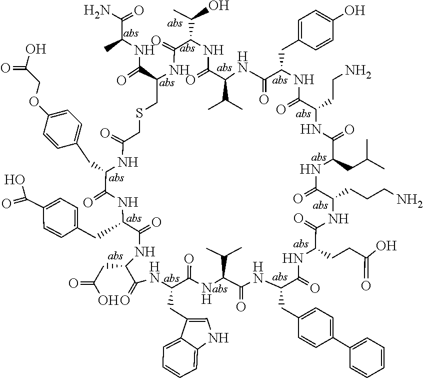 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1687 was prepared on a 50 μmol scale. The yield of the product was 9.4 mg, and its estimated purity by LCMS analysis was 99.4%. Analysis condition B: Retention time=1.47 min; ESI-MS(+) m/z [M+2H]²⁺: 1044.2.

### Preparation of Compound 1688

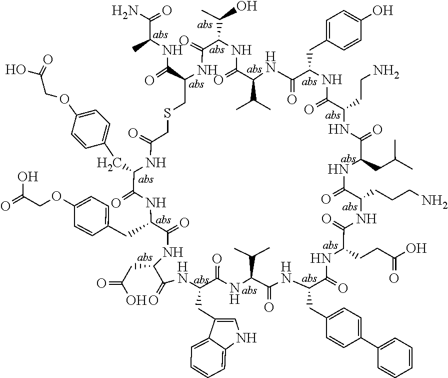 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](Cc2ccc(O)cc2)NC(=O)[C@H](CCN)NC1=O]

Compound 1688 was prepared on a 50 μmol scale. The yield of the product was 10.1 mg, and its estimated purity by LCMS analysis was 100%. Analysis condition A: Retention time=1.27 min; ESI-MS(+) m/z [M+2H]²⁺: 1059.2.

### Preparation of Compound 1689

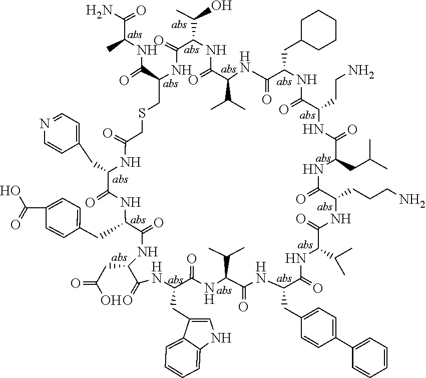 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1689 was prepared on a 50 μmol scale. The yield of the product was 11.6 mg, and its estimated purity by LCMS analysis was 86.4%. Analysis condition A: Retention time=1.73 min; ESI-MS(+) m/z [M+H]⁺: 1974.2.

### Preparation of Compound 1690

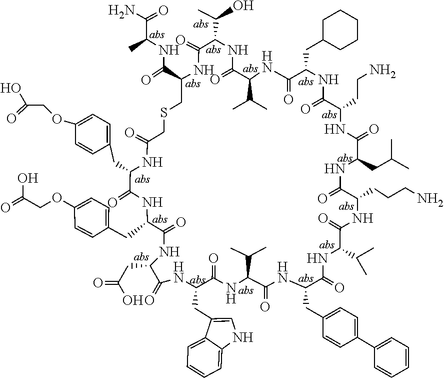 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1690 was prepared on a 50 μmol scale. The yield of the product was 9.7 mg, and its estimated purity by LCMS analysis was 98.6%. Analysis condition B: Retention time=1.76 min; ESI-MS(+) m/z [M+2H] 2+

### Preparation of Compound 1691

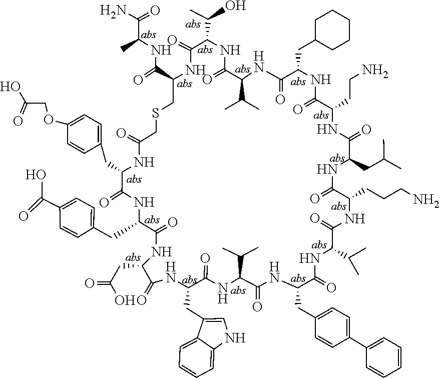 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1691 was prepared on a 50 μmol scale. The yield of the product was 9.9 mg, and its estimated purity by LCMS analysis was 93.1%. Analysis condition A: Retention time=1.81 min; ESI-MS(+) m/z [M+2H]²⁺: 1024.1.

### Preparation of Compound 1692

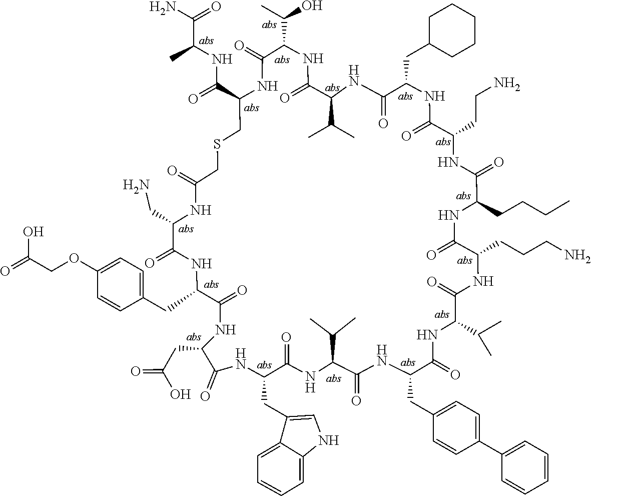 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1692 was prepared on a 50 μmol scale. The yield of the product was 14.6 mg, and its estimated purity by LCMS analysis was 85.6%. Analysis condition A: Retention time=1.67 min; ESI-MS(+) m/z [M+H]⁺: 1942.2.

### Preparation of Compound 1693

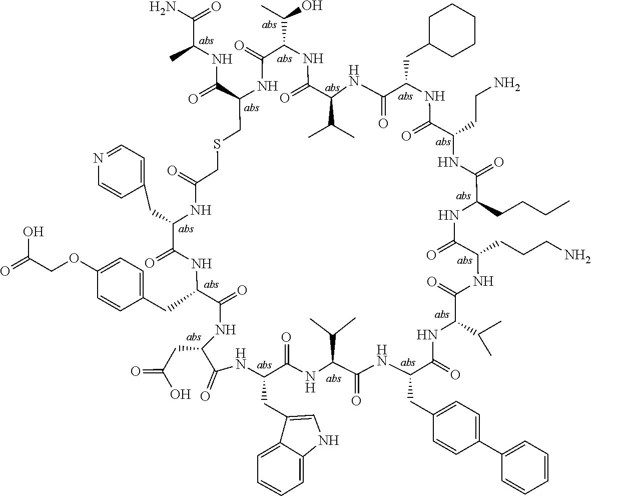 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1693 was prepared on a 50 μmol scale. The yield of the product was 16.4 mg, and its estimated purity by LCMS analysis was 100%. Analysis condition A: Retention time=1.79 min; ESI-MS(+) m/z [M+2H]²⁺: 1003.

### Preparation of Compound 1694

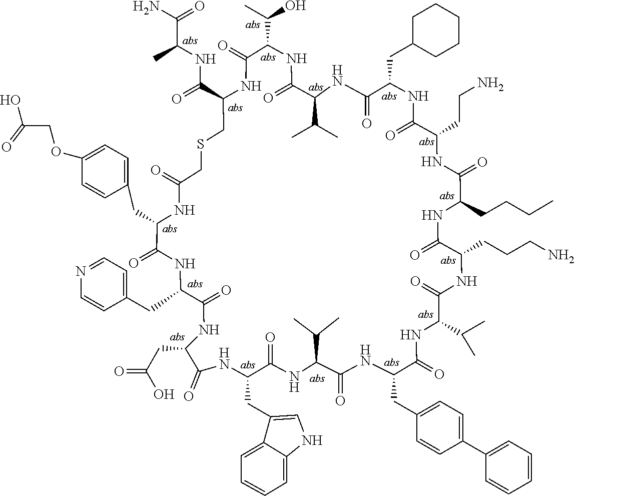 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1694 was prepared on a 50 μmol scale. The yield of the product was 11.1 mg, and its estimated purity by LCMS analysis was 86%. Analysis condition A: Retention time=2.03 min; ESI-MS(+) m/z [M+2H]²⁺: 1002.5.

### Preparation of Compound 1695

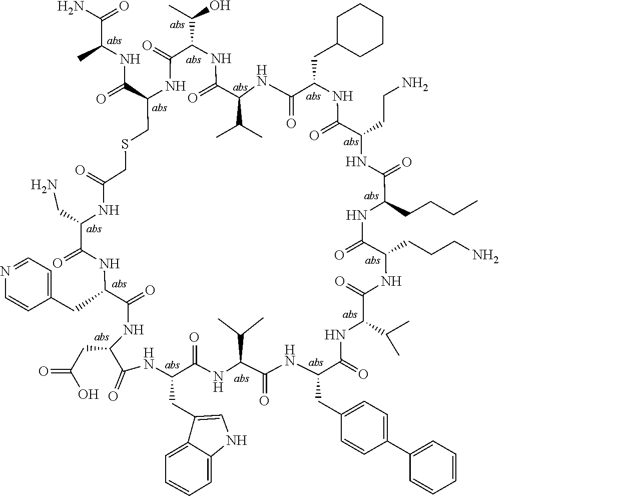 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1695 was prepared on a 50 μmol scale. The yield of the product was 4.5 mg, and its estimated purity by LCMS analysis was 97.6%. Analysis condition B: Retention time=1.51 min; ESI-MS(+) m/z [M+H]⁺: 1868.8.

### Preparation of Compound 1696

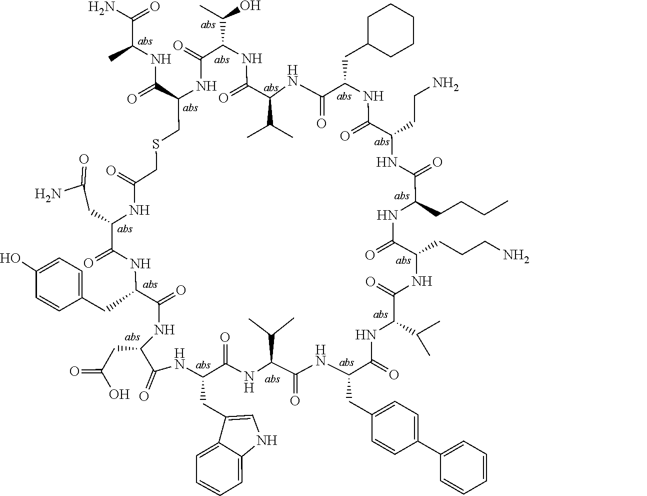 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1696 was prepared on a 50 μmol scale. The yield of the product was 32 mg, and its estimated purity by LCMS analysis was 89%. Analysis condition A: Retention time=1.9 min; ESI-MS(+) m/z [M+H]⁺: 1912.

### Preparation of Compound 1697

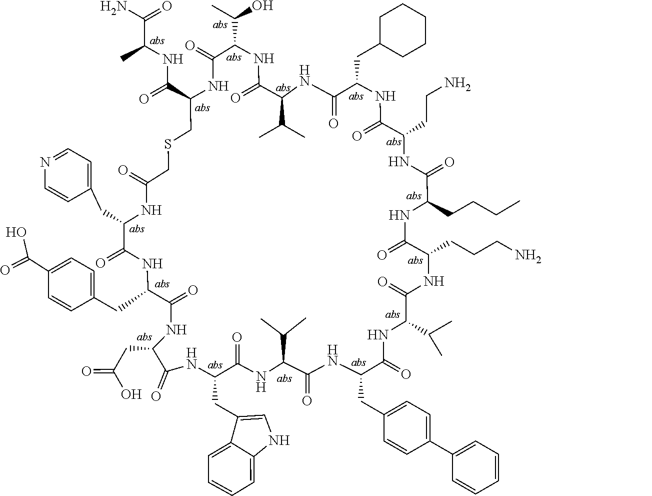 [SMILES: CCCC[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1697 was prepared on a 50 μmol scale. The yield of the product was 11.5 mg, and its estimated purity by LCMS analysis was 91.9%. Analysis condition B: Retention time=1.56 min; ESI-MS(+) m/z [M+2H]²⁺: 1017.

### Preparation of Compound 1698

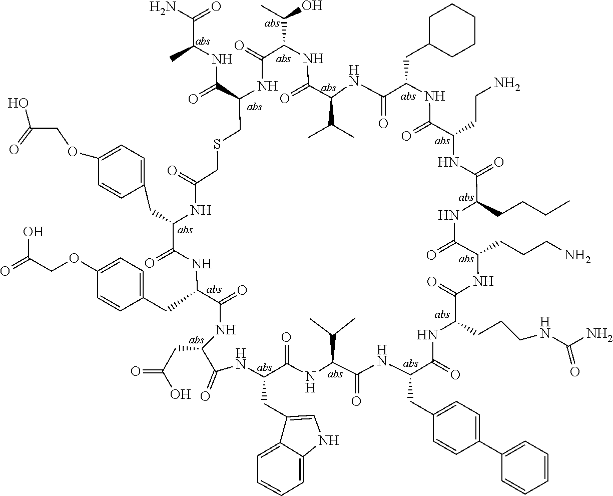 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1698 was prepared on a 50 μmol scale. The yield of the product was 17.4 mg, and its estimated purity by LCMS analysis was 92.6%. Analysis condition A: Retention time=1.6 min; ESI-MS(+) m/z [M+2H]²⁺: 1068.

### Preparation of Compound 1699

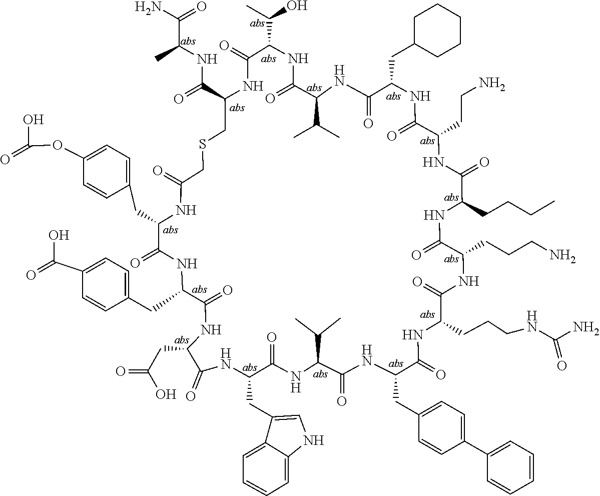 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1699 was prepared on a 50 μmol scale. The yield of the product was 3.2 mg, and its estimated purity by LCMS analysis was 92%. Analysis condition A: Retention time=1.62 min; ESI-MS(+) m/z [M+2H]²⁺: 1053.2.

Pronation of Command 1700

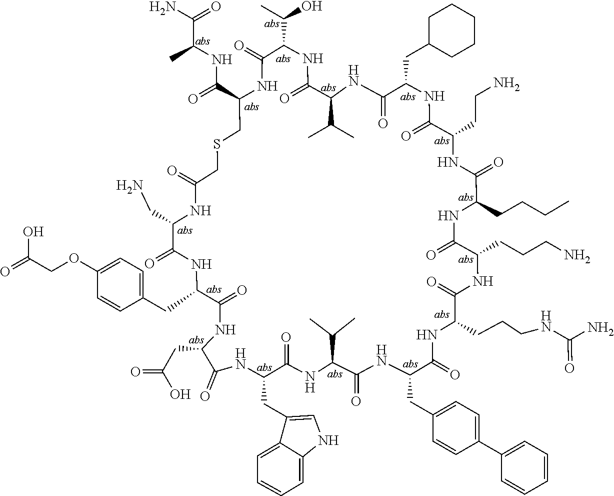 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1700 was prepared on a 50 μmol scale. The yield of the product was 5.1 mg, and its estimated purity by LCMS analysis was 99.2%. Analysis condition B: Retention time=1.5 min; ESI-MS(+) m/z [M+2H]²⁺: 1000.9.

### Preparation of Compound 1701

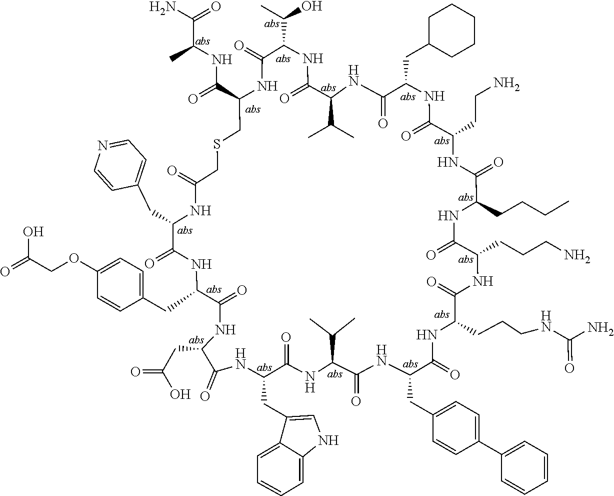 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1701 was prepared on a 50 μmol scale. The yield of the product was 5.8 mg, and its estimated purity by LCMS analysis was 93.9%. Analysis condition B: Retention time=1.48 min; ESI-MS(+) m/z [M+2H]²⁺: 1031.5.

### Preparation of Compound 1702

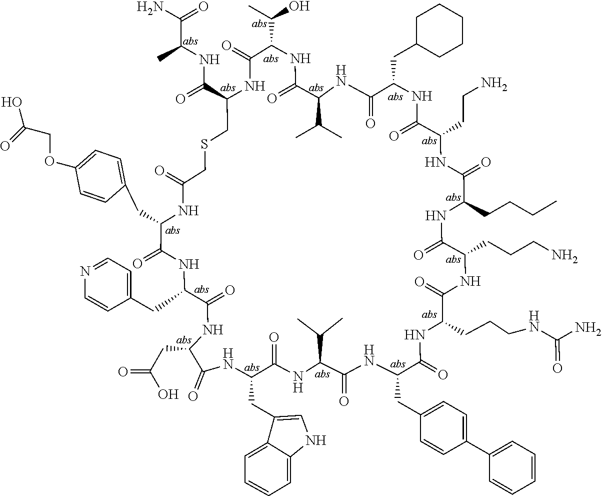 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1702 was prepared on a 50 μmol scale. The yield of the product was 16.4 mg, and its estimated purity by LCMS analysis was 87.7%. Analysis condition B: Retention time=1.43 min; ESI-MS(+) m/z [M+3H]³⁺: 688.2.

### Preparation of Compound 1703

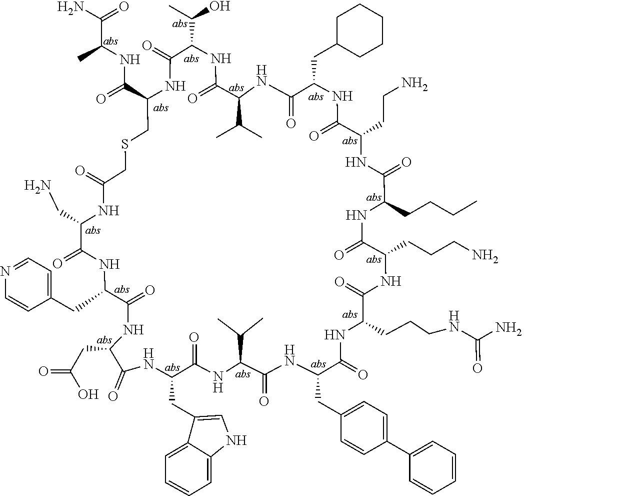 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1703 was prepared on a 50 μmol scale. The yield of the product was 9 mg, and its estimated purity by LCMS analysis was 90.8%. Analysis condition B: Retention time=1.38 min; ESI-MS(+) m/z [M+H]⁺: 1927.2.

### Preparation of Compound 1704

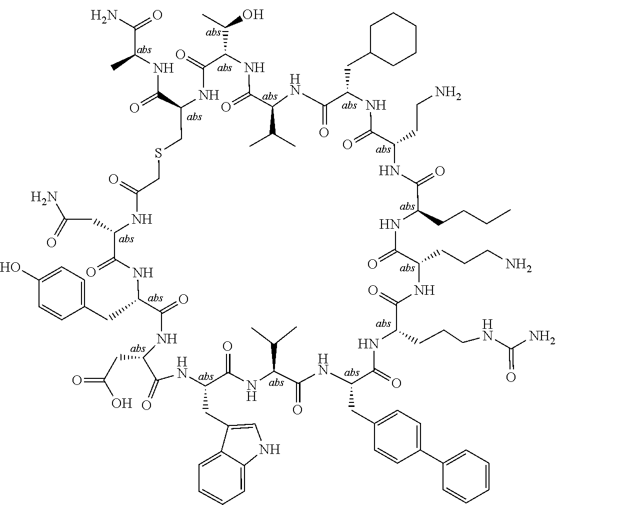 [SMILES: CCCC[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1704 was prepared on a 50 μmol scale. The yield of the product was 3.1 mg, and its estimated purity by LCMS analysis was 98%. Analysis condition A: Retention time=1.58 min; ESI-MS(+) m/z [M+2H]²⁺: 985.3.

### Preparation of Compound 1705

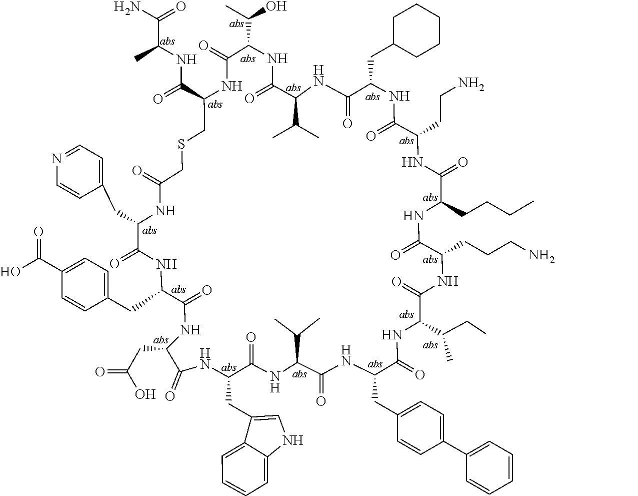 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1705 was prepared on a 50 μmol scale. The yield of the product was 17.9 mg, and its estimated purity by LCMS analysis was 89.3%. Analysis condition A: Retention time=1.77 min; ESI-MS(+) m/z [M+H]⁺: 1988.2.

### Preparation of Compound 1706

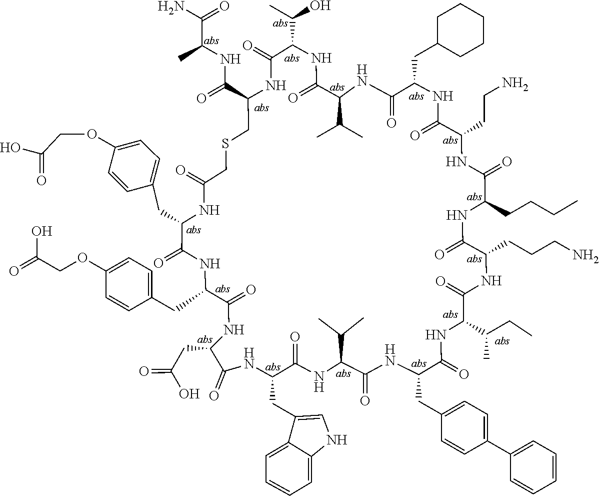 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1706 was prepared on a 50 μmol scale. The yield of the product was 17.5 mg, and its estimated purity by LCMS analysis was 88.6%. Analysis condition A: Retention time=1.81 min; ESI-MS(+) m/z [M+2H]²⁺: 1046.1.

### Preparation of Compound 1707

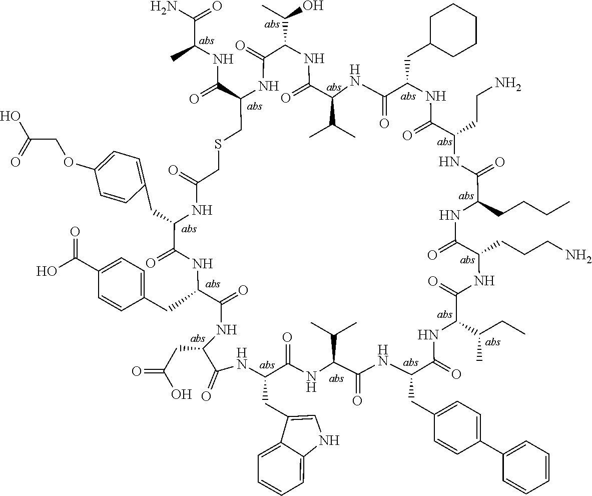 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1707 was prepared on a 50 μmol scale. The yield of the product was 24.5 mg, and its estimated purity by LCMS analysis was 93.5%. Analysis condition A: Retention time=1.92 min; ESI-MS(+) m/z [M+2H]²⁺: 1031.1.

### Preparation of Compound 1708

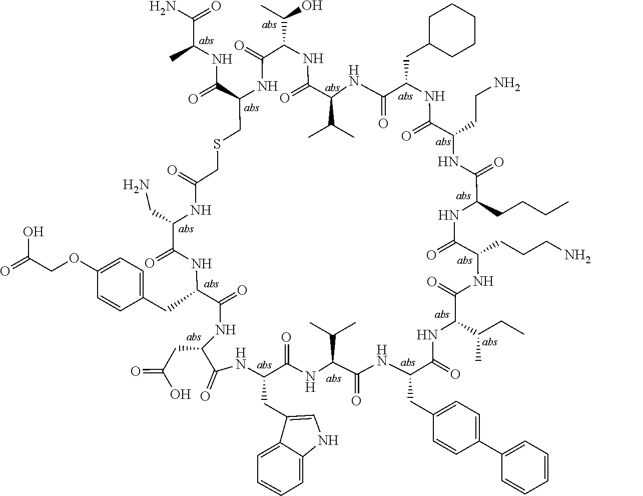 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1708 was prepared on a 50 μmol scale. The yield of the product was 15.1 mg, and its estimated purity by LCMS analysis was 95.5%. Analysis condition A: Retention time=1.62 min; ESI-MS(+) m/z [M+H]⁺: 1957.2.

### Preparation of Compound 1709

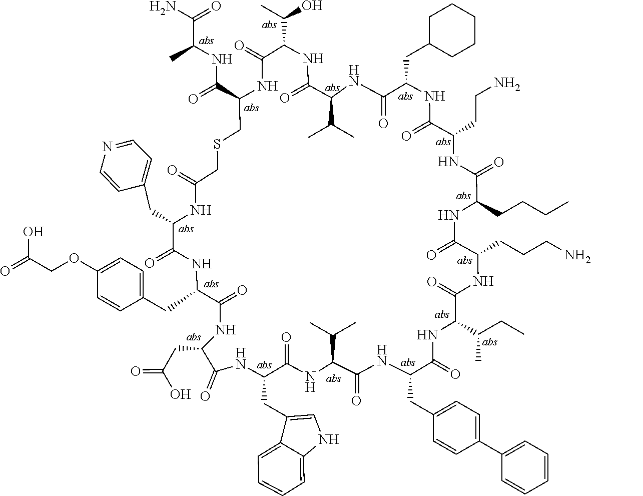 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1709 was prepared on a 50 μmol scale. The yield of the product was 3.4 mg, and its estimated purity by LCMS analysis was 97%. Analysis condition B: Retention time=1.54 min; ESI-MS(+) m/z [M+2H]²⁺: 1010.2.

### Preparation of Compound 1710

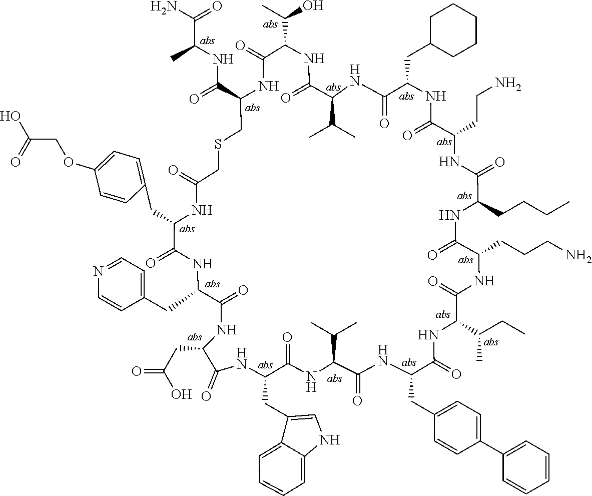 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1710 was prepared on a 50 μmol scale. The yield of the product was 20.2 mg, and its estimated purity by LCMS analysis was 89.5%. Analysis condition B: Retention time=1.61 min; ESI-MS(+) m/z [M+2H]²⁺: 1010.1.

### Preparation of Compound 1711

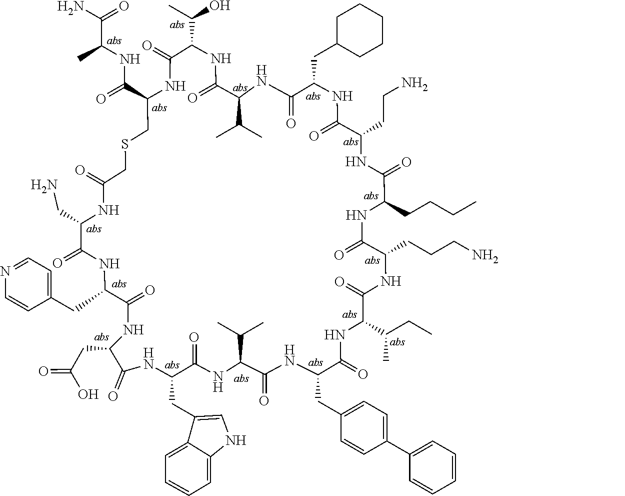 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1711 was prepared on a 50 μmol scale. The yield of the product was 16.2 mg, and its estimated purity by LCMS analysis was 93.1%. Analysis condition A: Retention time=1.76 min; ESI-MS(+) m/z [M+H]⁺: 1882.8.

### Preparation of Compound 1712

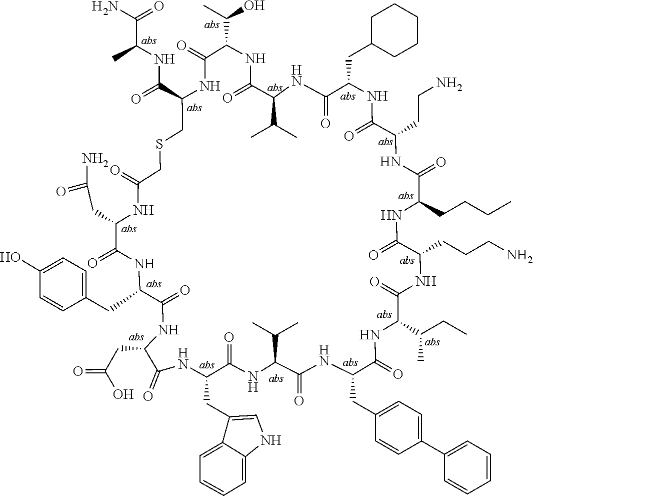 [SMILES: CCCC[C@H]1NC(=O)[C@H](CCCN)NC(=O)[C@H]([C@@H](C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1712 was prepared on a 50 μmol scale. The yield of the product was 14.6 mg, and its estimated purity by LCMS analysis was 94.4%. Analysis condition B: Retention time=1.65 min; ESI-MS(+) m/z [M+H]⁺: 1925.9.

### Preparation of Compound 1713

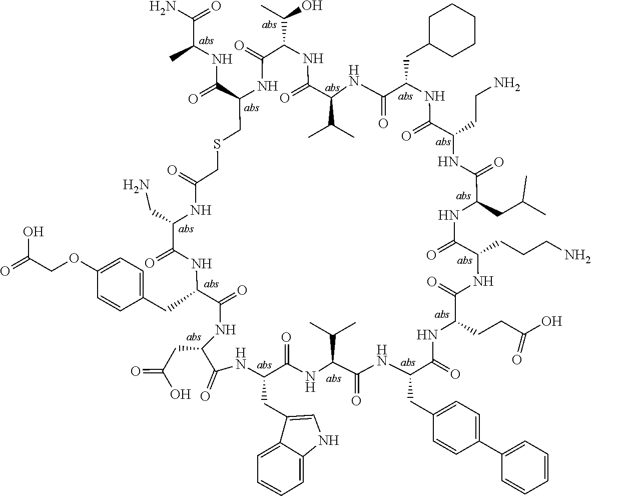 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1713 was prepared on a 50 μmol scale. The yield of the product was 10.9 mg, and its estimated purity by LCMS analysis was 94.2%. Analysis condition B: Retention time=1.6 min; ESI-MS(+) m/z [M+2H]²⁺: 980.

### Preparation of Compound 1714

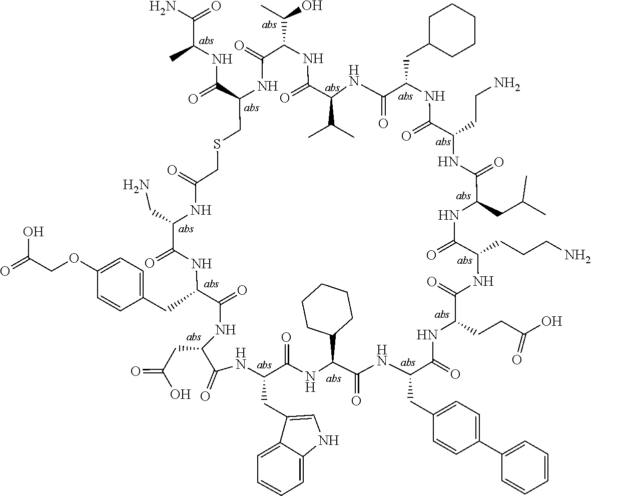 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1714 was prepared on a 50 μmol scale. The yield of the product was 15.2 mg, and its estimated purity by LCMS analysis was 97.8%. Analysis condition B: Retention time=1.55 min; ESI-MS(+) m/z [M+2H]²⁺: 1007.2.

### Preparation of Compound 1715

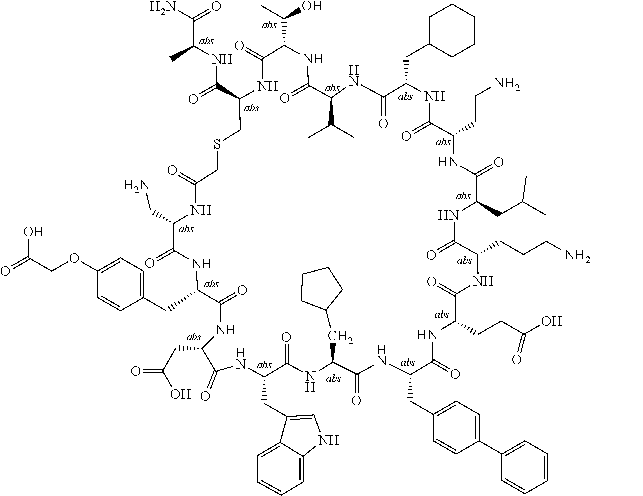 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](CC2CCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1715 was prepared on a 50 μmol scale. The yield of the product was 5.1 mg, and its estimated purity by LCMS analysis was 88.1%. Analysis condition B: Retention time=1.6 min; ESI-MS(+) m/z [M+2H]²⁺: 1007.

### Preparation of Compound 1716

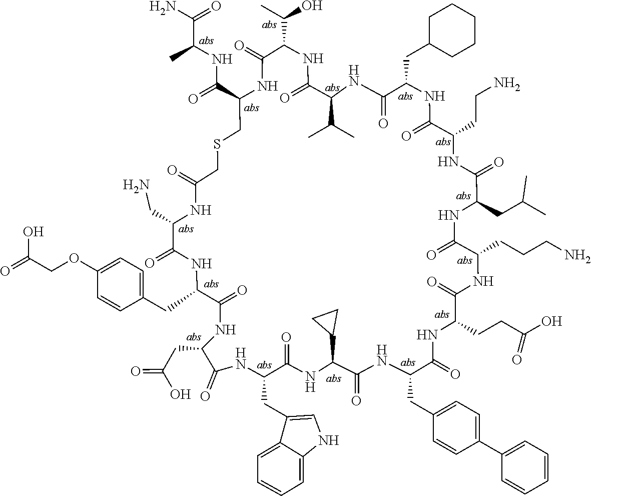 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C2CC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1716 was prepared on a 50 μmol scale. The yield of the product was 10.2 mg, and its estimated purity by LCMS analysis was 95.8%. Analysis condition B: Retention time=1.51 min; ESI-MS(+) m/z [M+H]⁺: 1969.8.

### Preparation of Compound 1717

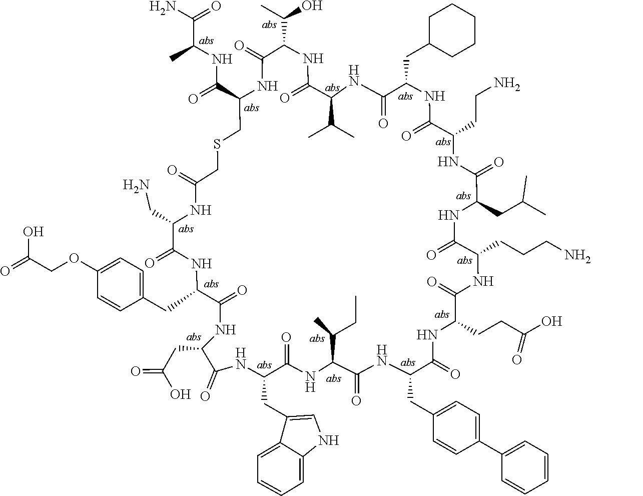 [SMILES: CC[C@H](C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC1=O]

Compound 1717 was prepared on a 50 μmol scale. The yield of the product was 11.3 mg, and its estimated purity by LCMS analysis was 92.6%. Analysis condition B: Retention time=1.45 min; ESI-MS(+) m/z [M+2H]²⁺: 1986.

### Preparation of Compound 1718

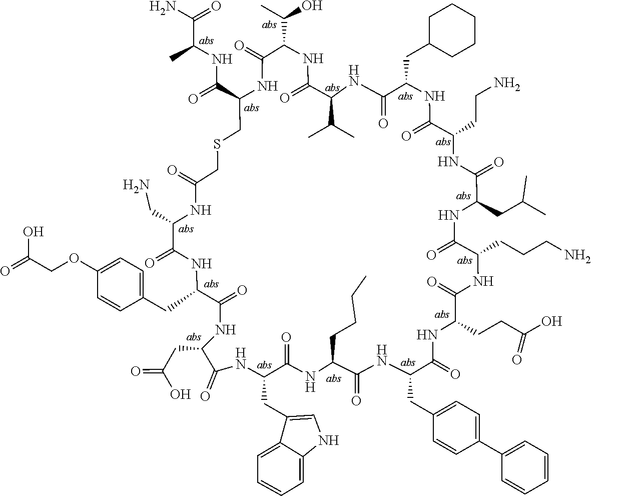 [SMILES: CCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC1=O]

Compound 1718 was prepared on a 50 μmol scale. The yield of the product was 31.6 mg, and its estimated purity by LCMS analysis was 100%. Analysis condition A: Retention time=1.56 min; ESI-MS(+) m/z [M+2H]²⁺: 994.3.

### Preparation of Compound 1719

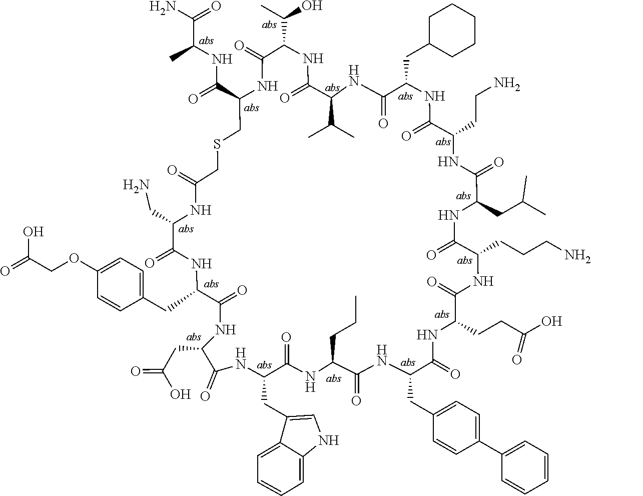 [SMILES: CCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC1=O]

Compound 1719 was prepared on a 50 μmol scale. The yield of the product was 21.2 mg, and its estimated purity by LCMS analysis was 90.8%. Analysis condition B: Retention time=1.61 min; ESI-MS(+) m/z [M+2H]²⁺: 987.1.

### Preparation of Compound 1720

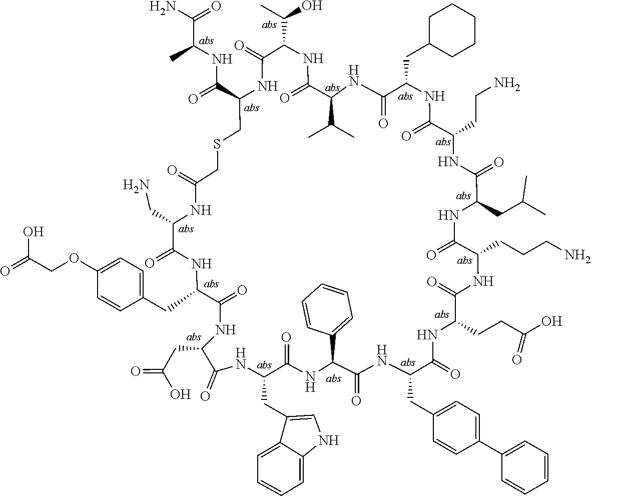 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1720 was prepared on a 50 μmol scale. The yield of the product was 12.6 mg, and its estimated purity by LCMS analysis was 85.8%. Analysis condition A: Retention time=1.68 min; ESI-MS(+) m/z [M+2H]²⁺: 1004.1.

### Preparation of Compound 1721

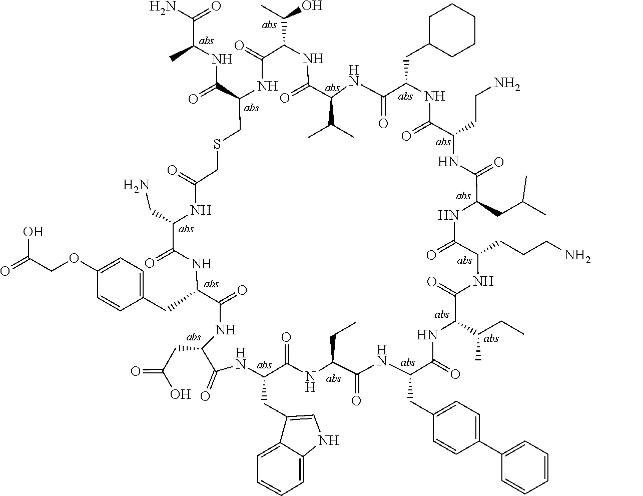 [SMILES: CC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC1=O]

Compound 1721 was prepared on a 50 μmol scale. The yield of the product was 8.4 mg, and its estimated purity by LCMS analysis was 100%. Analysis condition A: Retention time=1.68 min; ESI-MS(+) m/z [M+2H]²⁺: 1942.1.

### Preparation of Compound 1722

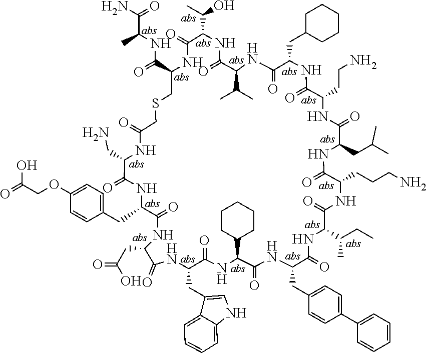 [SMILES: CC[C@H](C)[C@@H]1NC(=O)[C@H](Cc2ccc(-c3ccccc3)cc2)NC(=O)[C@H](C2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1722 was prepared on a 50 μmol scale. The yield of the product was 15.6 mg, and its estimated purity by LCMS analysis was 93.1%. Analysis condition B: Retention time=1.63 min; ESI-MS(+) m/z [M+2H]²⁺: 1996.

### Preparation of Compound 1723

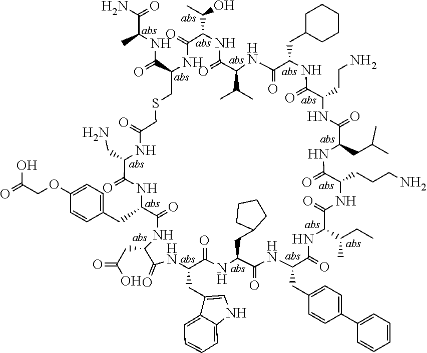 [SMILES: CC[C@H](C)[C@@H]1NC(=O)[C@H](Cc2ccc(-c3ccccc3)cc2)NC(=O)[C@H](CC2CCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1723 was prepared on a 50 μmol scale. The yield of the product was 9 mg, and its estimated purity by LCMS analysis was 95.7%. Analysis condition A: Retention time=1.85 min; ESI-MS(+) m/z [M+2H]²⁺: 1997.

### Preparation of Compound 1724

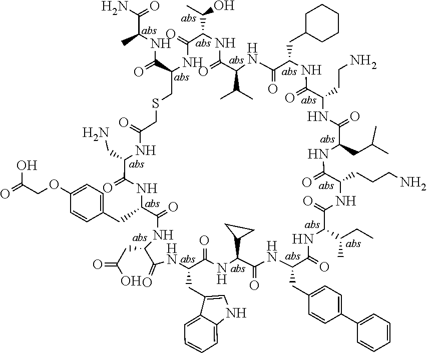 [SMILES: CC[C@H](C)[C@@H]1NC(=O)[C@H](Cc2ccc(-c3ccccc3)cc2)NC(=O)[C@H](C2CC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1724 was prepared on a 50 μmol scale. The yield of the product was 8.1 mg, and its estimated purity by LCMS analysis was 94.9%. Analysis condition B: Retention time=1.56 min; ESI-MS(+) m/z [M+H]⁺: 1954.2.

### Preparation of Compound 1725

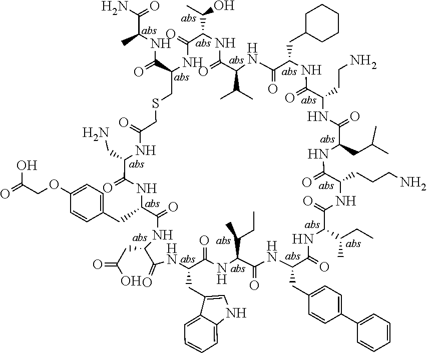 [SMILES: CC[C@H](C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC1=O]

Compound 1725 was prepared on a 50 μmol scale. The yield of the product was 11.5 mg, and its estimated purity by LCMS analysis was 95%. Analysis condition A: Retention time=1.8 min; ESI-MS(+) m/z [M+H]⁺: 1970.1.

### Preparation of Compound 1726

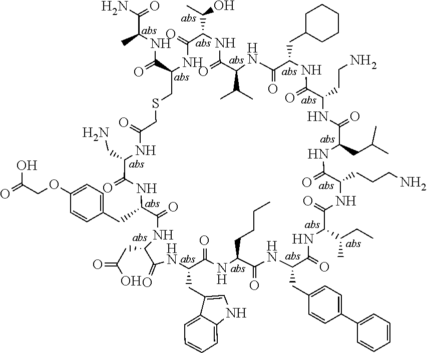 [SMILES: CCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC1=O]

Compound 1726 was prepared on a 50 μmol scale. The yield of the product was 15.7 mg, and its estimated purity by LCMS analysis was 100%. Analysis condition A: Retention time=1.85 min; ESI-MS(+) m/z [M+H]⁺: 1970.1.

### Preparation of Compound 1727

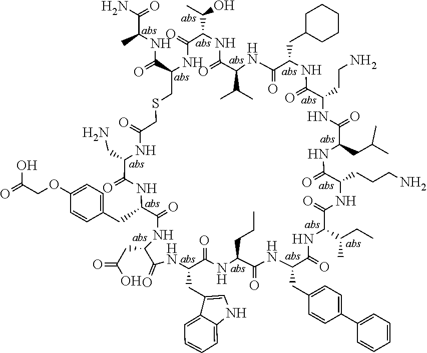 [SMILES: CCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H]([C@@H](C)CC)NC(=O)[C@H](Cc2ccc(-c3ccccc3)cc2)NC1=O]

Compound 1727 was prepared on a 50 μmol scale. The yield of the product was 21.6 mg, and its estimated purity by LCMS analysis was 85.1%. Analysis condition B: Retention time=1.65 min; ESI-MS(+) m/z [M+H]⁺: 1956.

### Preparation of Compound 1728

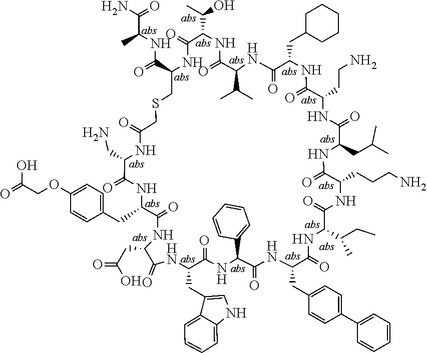 [SMILES: CC[C@H](C)[C@@H]1NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 1728 was prepared on a 50 μmol scale. The yield of the product was 5.5 mg, and its estimated purity by LCMS analysis was 100%. Analysis condition A: Retention time=1.83 min; ESI-MS(+) m/z [M+H]⁺: 1990.1.

### Preparation of Compound 1729

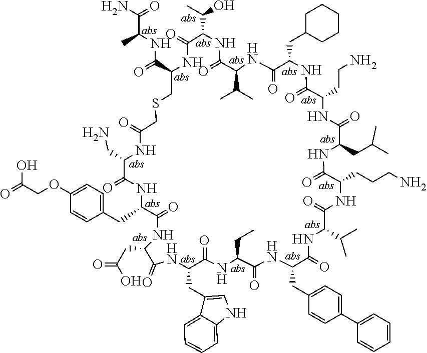

Compound 1729 was prepared on a 50 μmol scale. The yield of the product was 8.2 mg, and its estimated purity by LCMS analysis was 90.2%. Analysis condition A: Retention time=1.61 min; ESI-MS(+) m/z [M+2H]²⁺: 964.8.

### Preparation of Compound 1730

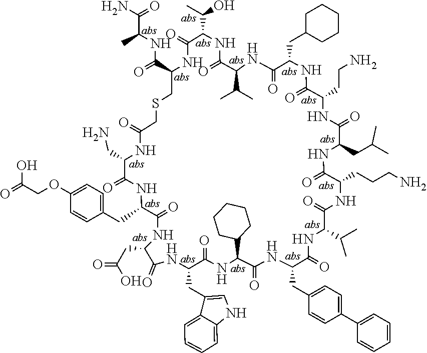

Compound 1730 was prepared on a 50 μmol scale. The yield of the product was 1.7 mg, and its estimated purity by LCMS analysis was 90.6%. Analysis condition A: Retention time=1.76 min; ESI-MS(+) m/z [M+H]⁺: 1982.2.

### Preparation of Compound 1731

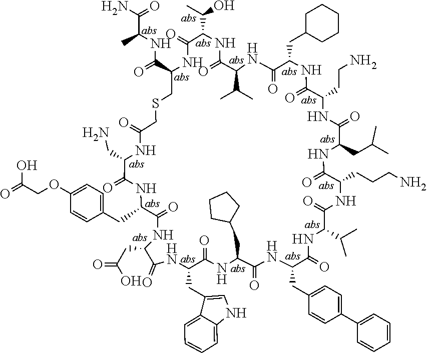

Compound 1731 was prepared on a 50 μmol scale. The yield of the product was 8.9 mg, and its estimated purity by LCMS analysis was 94.6%. Analysis condition A: Retention time=1.79 min; ESI-MS(+) m/z [M+H]⁺: 1981.8.

### Preparation of Compound 1732

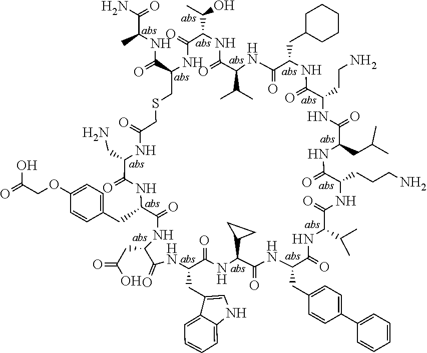 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C2CC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1732 was prepared on a 50 μmol scale. The yield of the product was 12.9 mg, and its estimated purity by LCMS analysis was 96.7%. Analysis condition B: Retention time=1.5 min; ESI-MS(+) m/z [M+H]⁺: 1940.

### Preparation of Compound 1733

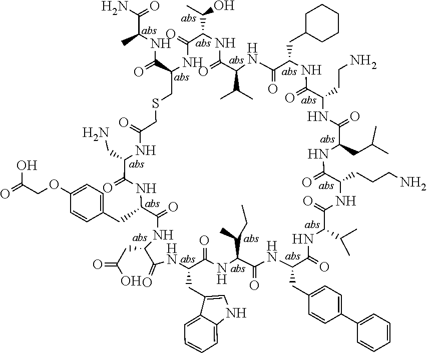 [SMILES: CC[C@H](C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1733 was prepared on a 50 μmol scale. The yield of the product was 8.8 mg, and its estimated purity by LCMS analysis was 90.1%. Analysis condition B: Retention time=1.55 min; ESI-MS(+) m/z [M+2H]²⁺: 978.9.

### Preparation of Compound 1734

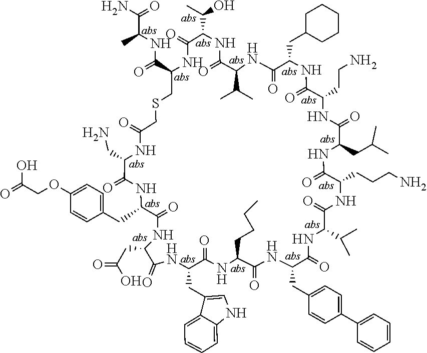 [SMILES: CCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1734 was prepared on a 50 μmol scale. The yield of the product was 26.2 mg, and its estimated purity by LCMS analysis was 94.8%. Analysis condition B: Retention time=1.57 min; ESI-MS(+) m/z [M+2H]²⁺: 979.

### Preparation of Compound 1735

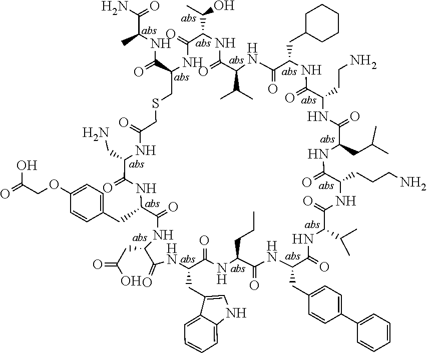 [SMILES: CCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 1735 was prepared on a 50 μmol scale. The yield of the product was 26 mg, and its estimated purity by LCMS analysis was 85.6%. Analysis condition B: Retention time=1.62 min; ESI-MS(+) m/z [M+2H]²⁺: 972.1.

### Preparation of Compound 1736

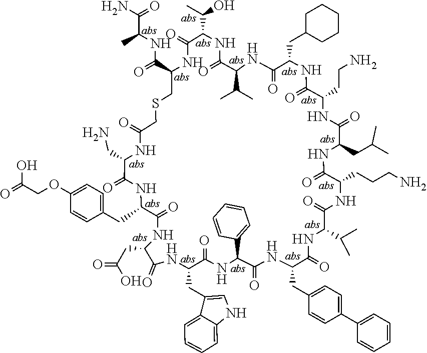 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1736 was prepared on a 50 μmol scale. The yield of the product was 10.3 mg, and its estimated purity by LCMS analysis was 96.1%. Analysis condition A: Retention time=1.77 min; ESI-MS(+) m/z [M+H]⁺: 1976.

### Preparation of Compound 1737

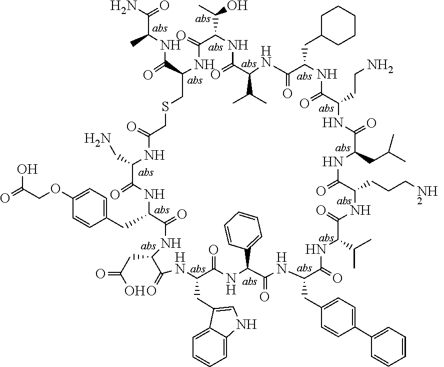 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](c2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 1737 was prepared on a 50 μmol scale. The yield of the product was 10.2 mg, and its estimated purity by LCMS analysis was 85.2%. Analysis condition A: Retention time=1.76 min; ESI-MS(+) m/z [M+2H]²⁺: 1004.1.

### Preparation of Compound 1738

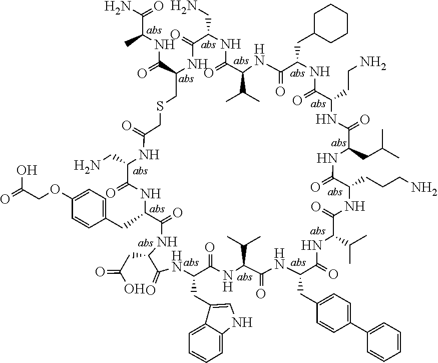 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CN)NC(=O)[C@H](C(C)C)NC(=O)[C@H](CC2CCCCC2)NC(=O)[C@H](CCN)NC1=O]

Compound 1738 was prepared on a 50 μmol scale. The yield of the product was 20 mg, and its estimated purity by LCMS analysis was 92.9%. Analysis condition B: Retention time=1.34 min; ESI-MS(+) m/z [M+3H]³⁺: 643.4.

### Preparation of Compound 1739

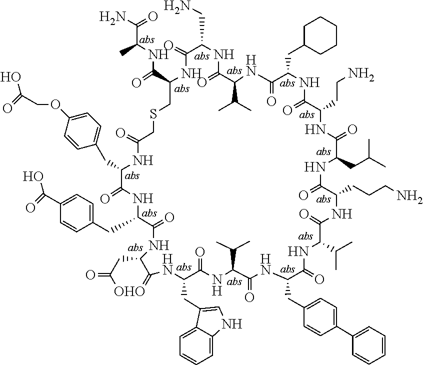 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CN)NC(=O)[C@H](C(C)C)NC(=O)[C@H](CC2CCCCC2)NC(=O)[C@H](CCN)NC1=O]

Compound 1739 was prepared on a 50 μmol scale. The yield of the product was 19.9 mg, and its estimated purity by LCMS analysis was 91.1%. Analysis condition A: Retention time=1.52 min; ESI-MS(+) m/z [M+2H]²⁺: 1017.1.

### Preparation of Compound 1740

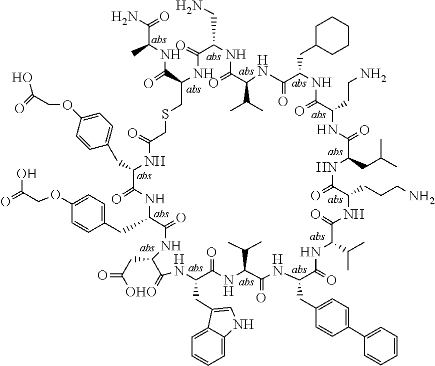 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CN)NC(=O)[C@H](C(C)C)NC(=O)[C@H](CC2CCCCC2)NC(=O)[C@H](CCN)NC1=O]

Compound 1740 was prepared on a 50 μmol scale. The yield of the product was 26.8 mg, and its estimated purity by LCMS analysis was 100%. Analysis condition A: Retention time=1.62 min; ESI-MS(+) m/z [M+2H]²⁺: 1032.1.

### Preparation of Compound 1741

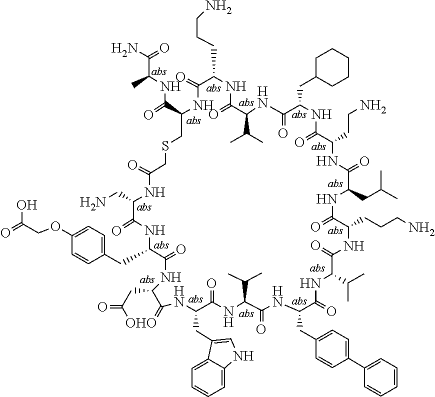 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CCCN)NC(=O)[C@H](C(C)C)NC(=O)[C@H](CC2CCCCC2)NC(=O)[C@H](CCN)NC1=O]

Compound 1741 was prepared on a 50 μmol scale. The yield of the product was 10.5 mg, and its estimated purity by LCMS analysis was 87.3%. Analysis condition B: Retention time=1.32 min; ESI-MS(+) m/z [M+3H]³⁺: 652.8.

### Preparation of Compound 1742

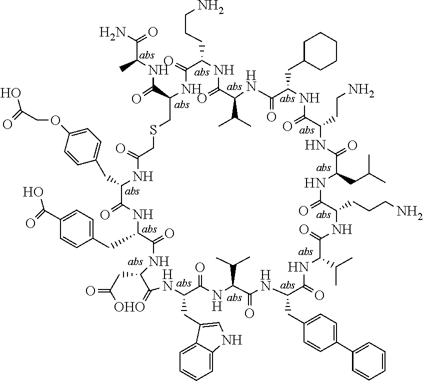 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCN)NC(=O)[C@H](C(C)C)NC(=O)[C@H](CC2CCCCC2)NC(=O)[C@H](CCN)NC1=O]

Compound 1742 was prepared on a 50 μmol scale. The yield of the product was 11.2 mg, and its estimated purity by LCMS analysis was 85.3%. Analysis condition A: Retention time=1.48 min; ESI-MS(+) m/z [M+2H]²⁺: 1031.2.

### Preparation of Compound 1743

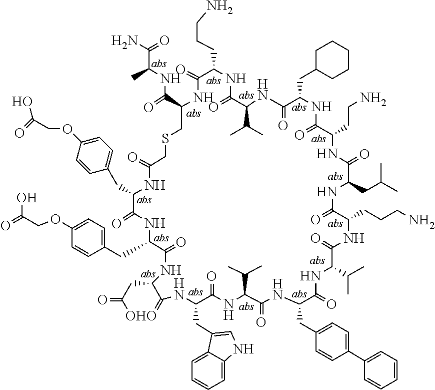 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCN)NC(=O)[C@H](C(C)C)NC(=O)[C@H](CC2CCCCC2)NC(=O)[C@H](CCN)NC1=O]

Compound 1743 was prepared on a 50 μmol scale. The yield of the product was 11.2 mg, and its estimated purity by LCMS analysis was 98.4%. Analysis condition A: Retention time=1.57 min; ESI-MS(+) m/z [M+2H]²⁺: 1046.

### Preparation of Compound 1744

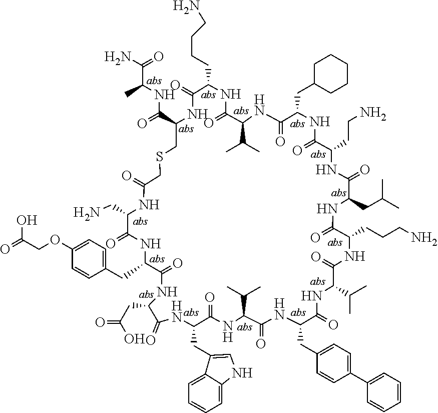 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CCCCN)NC(=O)[C@H](C(C)C)NC(=O)[C@H](CC2CCCCC2)NC(=O)[C@H](CCN)NC1=O]

Compound 1744 was prepared on a 50 μmol scale. The yield of the product was 12.4 mg, and its estimated purity by LCMS analysis was 94.1%. Analysis condition B: Retention time=1.4 min; ESI-MS(+) m/z [M+H]⁺: 1968.8.

### Preparation of Compound 1745

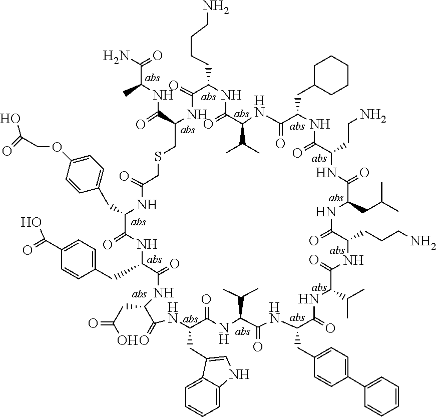 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCCN)NC(=O)[C@H](C(C)C)NC(=O)[C@H](CC2CCCCC2)NC(=O)[C@H](CCN)NC1=O]

Compound 1745 was prepared on a 50 μmol scale. The yield of the product was 16.8 mg, and its estimated purity by LCMS analysis was 93.4%. Analysis condition A: Retention time=1.49 min; ESI-MS(+) m/z [M+2H]²⁺: 1037.8.

### Preparation of Compound 1746

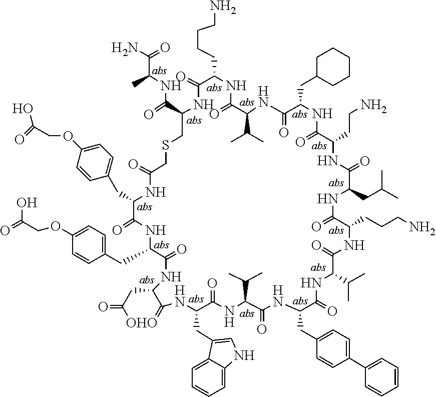 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCCN)NC(=O)[C@H](C(C)C)NC(=O)[C@H](CC2CCCCC2)NC(=O)[C@H](CCN)NC1=O]

Compound 1746 was prepared on a 50 μmol scale. The yield of the product was 14.3 mg, and its estimated purity by LCMS analysis was 97.4%. Analysis condition B: Retention time=1.49 min; ESI-MS(+) m/z [M+2H]²⁺: 1052.6.

### Preparation of Compound 1747

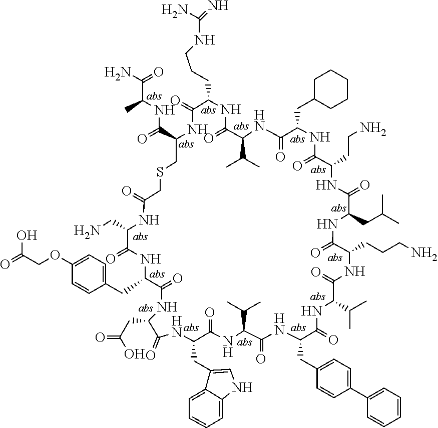 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CCCNC(=N)N)NC(=O)[C@H](C(C)C)NC(=O)[C@H](CC2CCCCC2)NC(=O)[C@H](CCN)NC1=O]

Compound 1747 was prepared on a 50 μmol scale. The yield of the product was 8.9 mg, and its estimated purity by LCMS analysis was 92.7%. Analysis condition B: Retention time=1.31 min; ESI-MS(+) m/z [M+2H]²⁺: 999.1.

### Preparation of Compound 1748

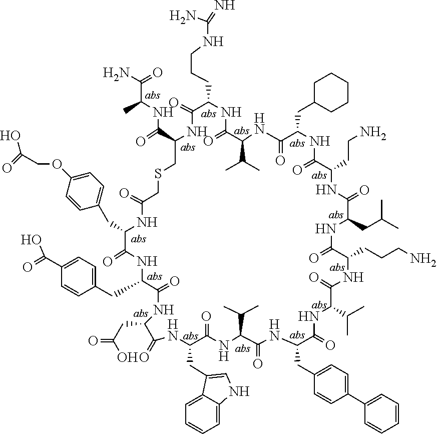 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCNC(=N)N)NC(=O)[C@H](C(C)C)NC(=O)[C@H](CC2CCCCC2)NC(=O)[C@H](CCN)NC1=O]

Compound 1748 was prepared on a 50 μmol scale. The yield of the product was 11.3 mg, and its estimated purity by LCMS analysis was 93%. Analysis condition A: Retention time=1.59 min; ESI-MS(+) m/z [M+2H]²⁺: 1052.1.

### Preparation of Compound 1749

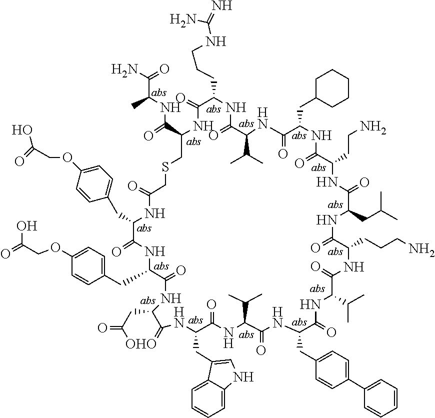 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CCCNC(=N)N)NC(=O)[C@H](C(C)C)NC(=O)[C@H](CC2CCCCC2)NC(=O)[C@H](CCN)NC1=O]

Compound 1749 was prepared on a 50 μmol scale. The yield of the product was 13.8 mg, and its estimated purity by LCMS analysis was 95.3%. Analysis condition B: Retention time=1.55 min; ESI-MS(+) m/z [M+2H]²⁺: 1067.2.

### Preparation of Compound 1750

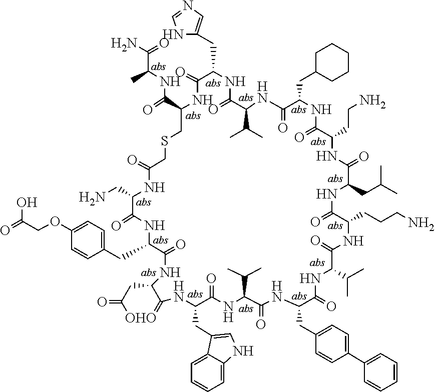 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](Cc2cnc[nH]2)NC(=O)[C@H](C(C)C)NC(=O)[C@H](CC2CCCCC2)NC(=O)[C@H](CCN)NC1=O]

Compound 1750 was prepared on a 50 μmol scale. The yield of the product was 15.4 mg, and its estimated purity by LCMS analysis was 99.2%. Analysis condition B: Retention time=1.39 min; ESI-MS(+) m/z [M+2H]²⁺: 989.9.

### Preparation of Compound 1751

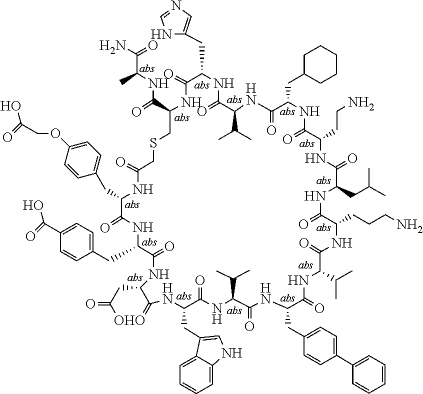 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](Cc2cnc[nH]2)NC(=O)[C@H](C(C)C)NC(=O)[C@H](CC2CCCCC2)NC(=O)[C@H](CCN)NC1=O]

Compound 1751 was prepared on a 50 μmol scale. The yield of the product was 17.9 mg, and its estimated purity by LCMS analysis was 90.9%. Analysis condition A: Retention time=1.54 min; ESI-MS(+) m/z [M+2H]²⁺: 1042.4.

### Preparation of Compound 1752

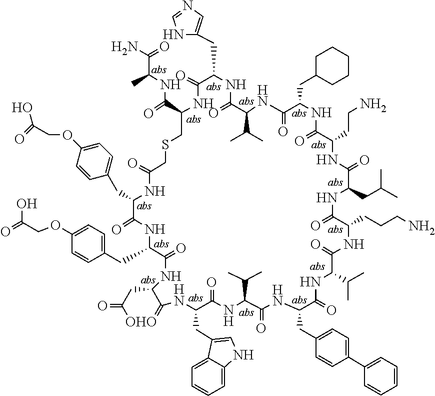 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](Cc2cnc[nH]2)NC(=O)[C@H](C(C)C)NC(=O)[C@H](CC2CCCCC2)NC(=O)[C@H](CCN)NC1=O]

Compound 1752 was prepared on a 50 μmol scale. The yield of the product was 28 mg, and its estimated purity by LCMS analysis was 96.2%. Analysis condition A: Retention time=1.63 min; ESI-MS(+) m/z [M+2H]²⁺: 1057.4.

### Preparation of Compound 1753

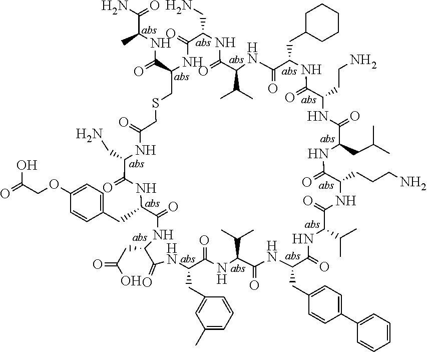 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H](CN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1753 was prepared on a 50 μmol scale. The yield of the product was 15 mg, and its estimated purity by LCMS analysis was 98.6%. Analysis condition A: Retention time=1.5 min; ESI-MS(+) m/z [M+2H]²⁺: 952.1.

### Preparation of Compound 1754

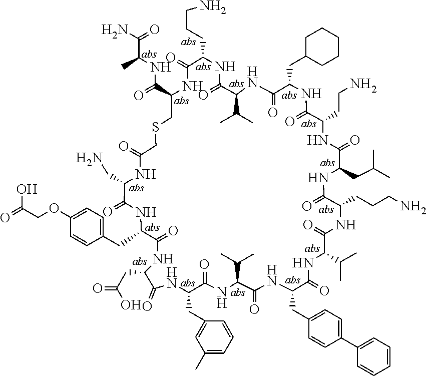 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1754 was prepared on a 50 μmol scale. The yield of the product was 7.8 mg, and its estimated purity by LCMS analysis was 95.3%. Analysis condition B: Retention time=1.33 min; ESI-MS(+) m/z [M+3H]³⁺: 644.4.

### Preparation of Compound 1755

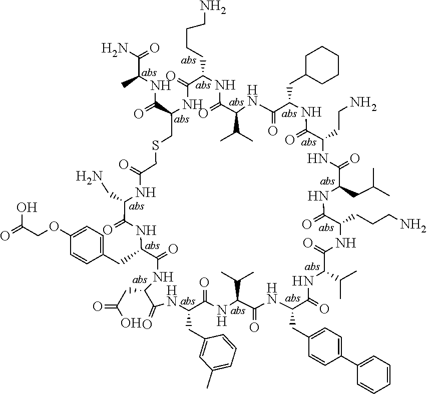 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H](CCCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1755 was prepared on a 50 μmol scale. The yield of the product was 10.1 mg, and its estimated purity by LCMS analysis was 88.9%. Analysis condition B: Retention time=1.41 min; ESI-MS(+) m/z [M+2H]²⁺: 973.1.

### Preparation of Compound 1756

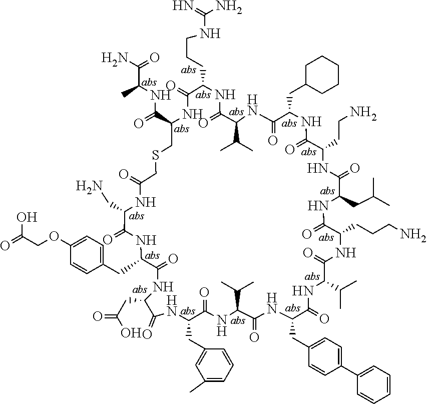 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H](CCCNC(=N)N)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1756 was prepared on a 50 μmol scale. The yield of the product was 9.8 mg, and its estimated purity by LCMS analysis was 100%. Analysis condition A: Retention time=1.55 min; ESI-MS(+) m/z [M+H]⁺: 1973.4.

### Preparation of Compound 1757

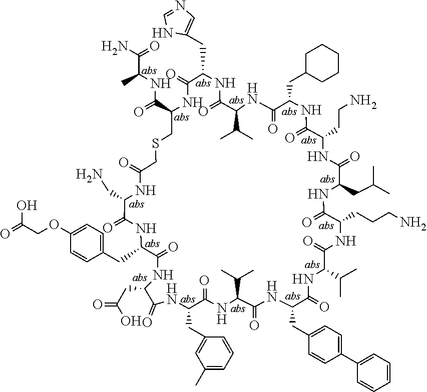 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 1757 was prepared on a 50 μmol scale. The yield of the product was 9.6 mg, and its estimated purity by LCMS analysis was 90.6%. Analysis condition A: Retention time=1.57 min; ESI-MS(+) m/z [M+2H]²⁺: 977.4.

### Preparation of Compound 1758

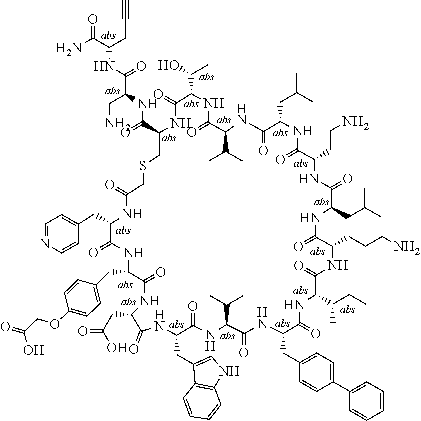

Compound 1758 was prepared on a 50 μmol scale. The yield of the product was 11.9 mg, and its estimated purity by LCMS analysis was 92.5%. Analysis condition B: Retention time=1.48 min; ESI-MS(+) m/z [M+2H]²⁺: 1044.2.

### Preparation of Compound 1759

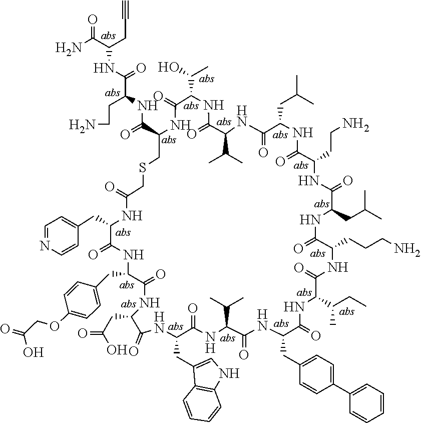

Compound 1759 was prepared on a 50 μmol scale. The yield of the product was 24.7 mg, and its estimated purity by LCMS analysis was 88.1%. Analysis condition A: Retention time=1.49 min; ESI-MS(+) m/z [M+2H]²⁺: 1052.

### Preparation of Compound 1760

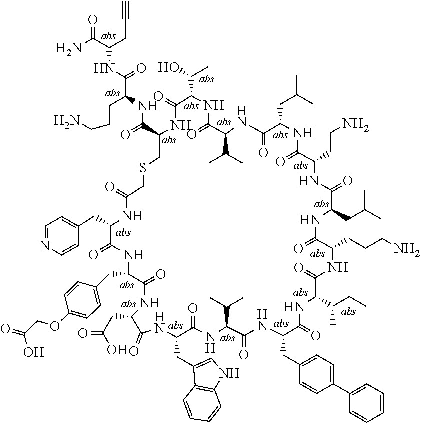

Compound 1760 was prepared on a 50 μmol scale. The yield of the product was 5.4 mg, and its estimated purity by LCMS analysis was 93.7%. Analysis condition B: Retention time=1.4, 1.47 min; ESI-MS(+) m/z [M+2H]²⁺: 1059.3.

### Preparation of Compound 1761

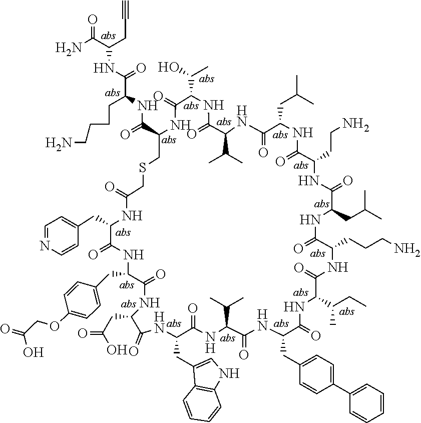

Compound 1761 was prepared on a 50 μmol scale. The yield of the product was 4.1 mg, and its estimated purity by LCMS analysis was 90.4%. Analysis condition A: Retention time=1.49 min; ESI-MS(+) m/z [M+2H]²⁺: 1066.1.

### Preparation of Compound 1762

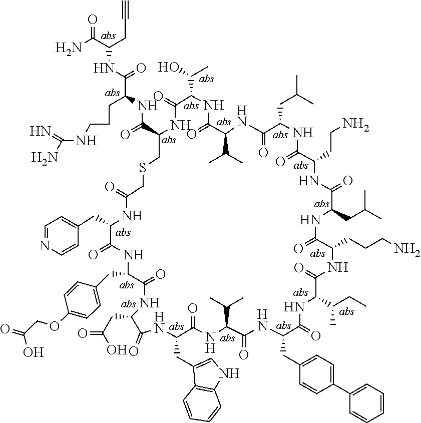

Compound 1762 was prepared on a 50 μmol scale. The yield of the product was 12.2 mg, and its estimated purity by LCMS analysis was 88.1%. Analysis condition A: Retention time=1.5 min; ESI-MS(+) m/z [M+2H]²⁺: 1080.1.

### Preparation of Compound 1763

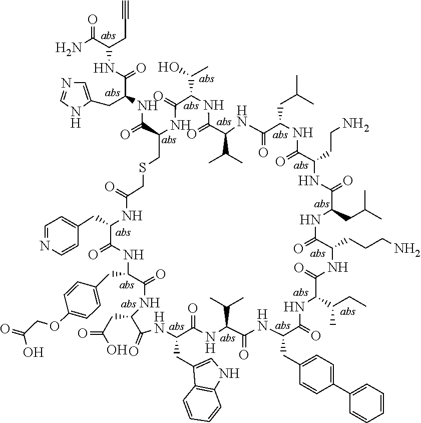

Compound 1763 was prepared on a 50 μmol scale. The yield of the product was 3.9 mg, and its estimated purity by LCMS analysis was 86%. Analysis condition B: Retention time=1.44 min; ESI-MS(+) m/z [M+2H]²⁺: 1070.2.

### Preparation of Compound 1764

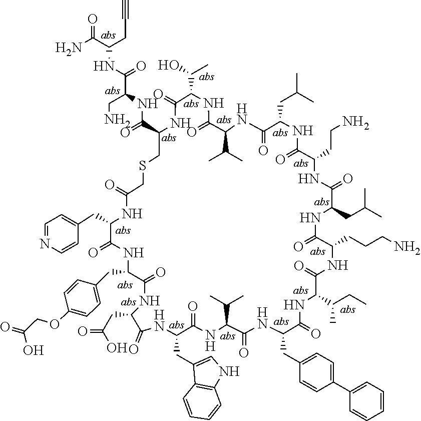

Compound 1764 was prepared on a 50 μmol scale. The yield of the product was 8.2 mg, and its estimated purity by LCMS analysis was 90.3%. Analysis condition B: Retention time=1.5 min; ESI-MS(+) m/z [M+2H]²⁺: 1045.8.

### Preparation of Compound 1765

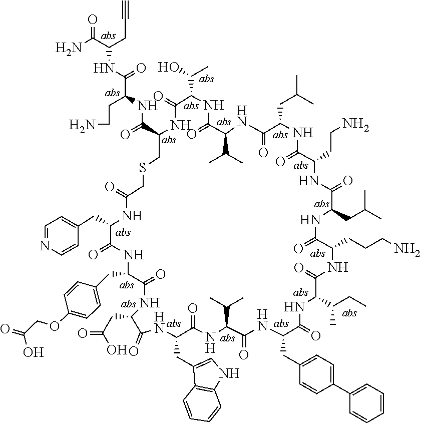

Compound 1765 was prepared on a 50 μmol scale. The yield of the product was 9 mg, and its estimated purity by LCMS analysis was 77.2%. Analysis condition B: Retention time=1.48 min; ESI-MS(+) m/z [M+3H]³⁺: 702.2.

### Preparation of Compound 1766

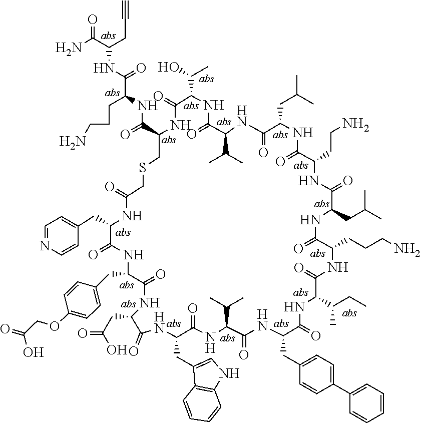

Compound 1766 was prepared on a 50 μmol scale. The yield of the product was 7.4 mg, and its estimated purity by LCMS analysis was 88.6%. Analysis condition A: Retention time=1.43 min; ESI-MS(+) m/z [M+2H]²⁺: 1059.4.

### Preparation of Compound 1767

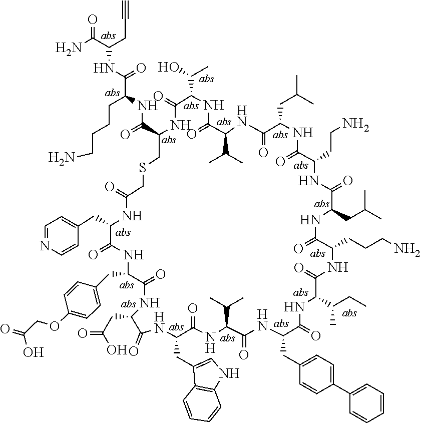

Compound 1767 was prepared on a 50 μmol scale. The yield of the product was 8.4 mg, and its estimated purity by LCMS analysis was 86.1%. Analysis condition B: Retention time=1.46 min; ESI-MS(+) m/z [M+3H]³⁺: 711.7.

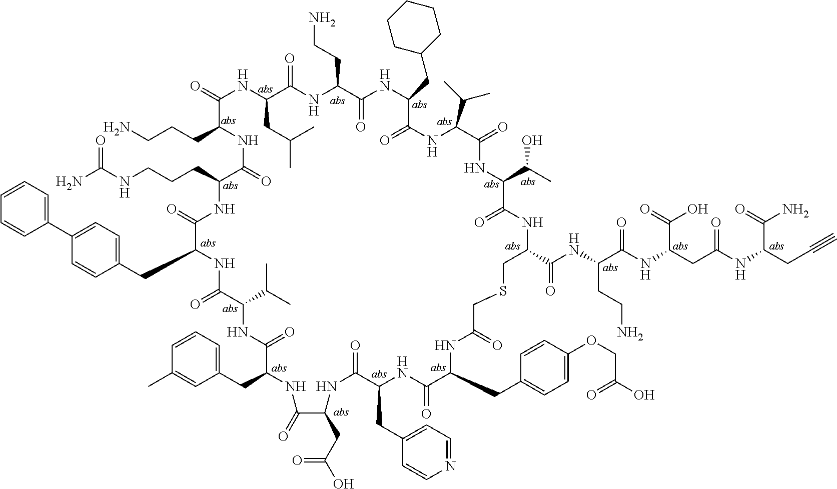 [SMILES: C#CC[C@H](NC(=O)C[C@H](NC(=O)[C@H](CCN)NC(=O)[C@@H]1CSCC(=O)N[C@@H](Cc2ccc(OCC(=O)O)cc2)C(=O)N[C@@H](Cc2ccncc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O)C(N)=O]

Compound 1768 was prepared on a 50 μmol scale. The yield of the product was 19.8 mg, and its estimated purity by LCMS analysis was 100%. Analysis condition A: Retention time=1.61 min; ESI-MS(+) m/z [M+2H]²⁺: 1139.

### Preparation of Compound 1769

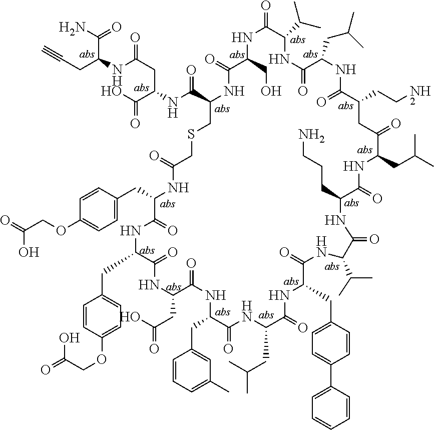 [SMILES: C#CC[C@H](NC(=O)C[C@H](NC(=O)[C@@H]1CSCC(=O)N[C@@H](Cc2ccc(OCC(=O)O)cc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](CC(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)C[C@@H](CCN)C(=O)N[C@@H](CC(C)C)C(=O)N[C@@H](C(C)C)C(=O)N[C@@H](CO)C(=O)N1)C(=O)O)C(N)=O]

Compound 1769 was prepared on a 50 μmol scale. The yield of the product was 7.4 mg, and its estimated purity by LCMS analysis was 100%. Analysis condition A: Retention time=1.56 min; ESI-MS(+) m/z [M+2H]²⁺: 1076.4.

### Preparation of Compound 1770

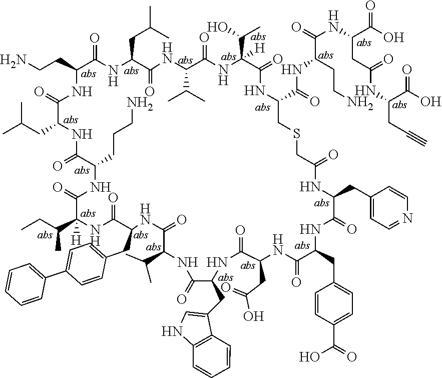 [SMILES: C#CC[C@H](NC(=O)C[C@H](NC(=O)[C@H](CCN)NC(=O)[C@@H]1CSCC(=O)N[C@@H](Cc2ccncc2)C(=O)N[C@@H](Cc2ccc(C(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC(C)C)C(=O)N[C@@H](C(C)C)C(=O)N[C@@H]([C@@H](C)O)C(=O)N1)C(=O)O)C(=O)O]

Compound 1770 was prepared on a 50 μmol scale. The yield of the product was 17.3 mg, and its estimated purity by LCMS analysis was 95.6%. Analysis condition A: Retention time=1.5 min; ESI-MS(+) m/z [M+2H]²⁺: 1095.1.

### Preparation of Compound 1771

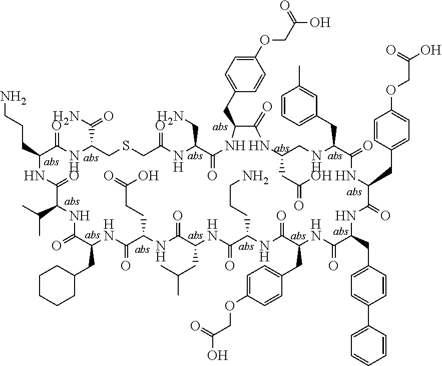 [SMILES: Cc1cccc(C[C@@H]2NC[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1771 was prepared on a 50 μmol scale. The yield of the product was 14.1 mg, and its estimated purity by LCMS analysis was 95.2%. Analysis condition B: Retention time=1.57 min; ESI-MS(+) m/z [M+2H]²⁺: 1067.2.

### Preparation of Compound 1772

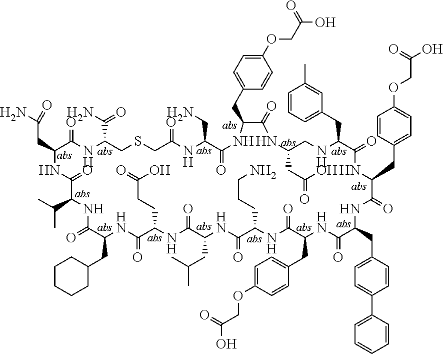 [SMILES: Cc1cccc(C[C@@H]2NC[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1772 was prepared on a 50 μmol scale. The yield of the product was 34 mg, and its estimated purity by LCMS analysis was 95.4%. Analysis condition: Retention time=1.74 min; ESI-MS(+) m/z [M+2H]²⁺: 1067.1.

### Preparation of Compound 1773

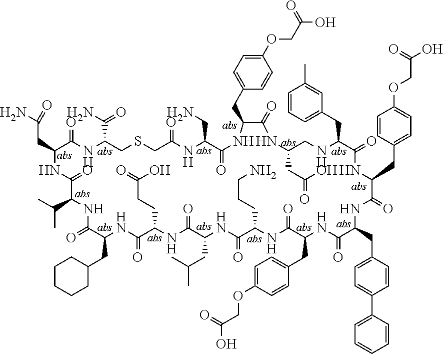 [SMILES: Cc1cccc(C[C@@H]2NC[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1773 was prepared on a 50 μmol scale. The yield of the product was 25 mg, and its estimated purity by LCMS analysis was 94.5%. Analysis condition A: Retention time=1.24 min; ESI-MS(+) m/z [M+2H]²⁺: 1067.2.

### Preparation of Compound 1774

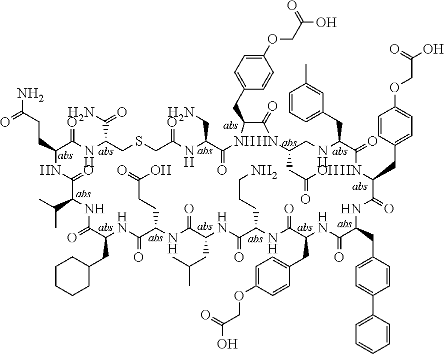 [SMILES: Cc1cccc(C[C@@H]2NC[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1774 was prepared on a 50 μmol scale. The yield of the product was 52.6 mg, and its estimated purity by LCMS analysis was 96.3%. Analysis condition B: Retention time=1.63 min; ESI-MS(+) m/z [M+2H]²⁺: 1074.2.

### Preparation of Compound 1775

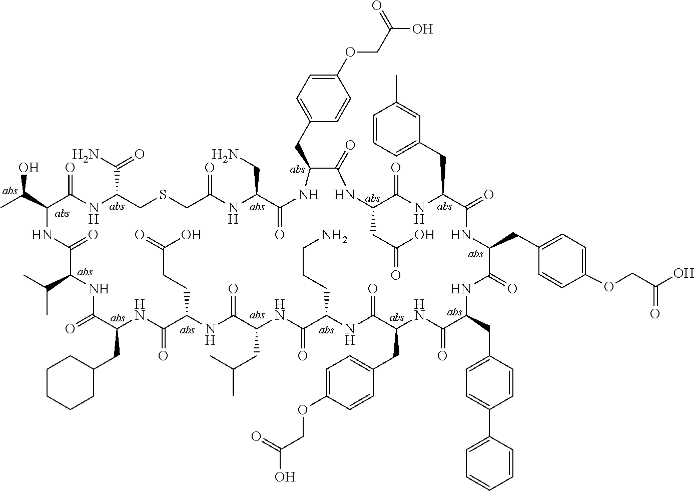 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1775 was prepared on a 50 μmol scale. The yield of the product was 13 mg, and its estimated purity by LCMS analysis was 91%. Analysis condition B: Retention time=1.71 min; ESI-MS(+) m/z [M+2H]²⁺: 1060.1.

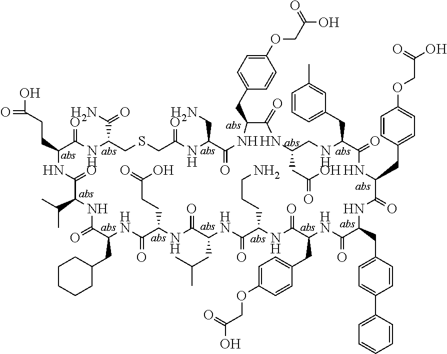 [SMILES: Cc1cccc(C[C@@H]2NC[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCC(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1776 was prepared on a 50 μmol scale. The yield of the product was 12.7 mg, and its estimated purity by LCMS analysis was 96%. Analysis condition B: Retention time=1.66 min; ESI-MS(+) m/z [M+2H]²⁺: 1072.2.

### Preparation of Compound 1777

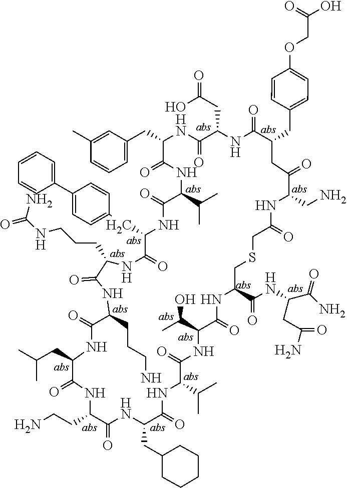 [SMILES: Cc1cccc(C[C@@H]2NC(=O)[C@H](CC(=O)O)NC(=O)[C@H](Cc3ccc(OCC(=O)O)cc3)CC(=O)[C@H](CN)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1777 was prepared on a 50 μmol scale. The yield of the product was 2.5 mg, and its estimated purity by LCMS analysis was 100%. Analysis condition: Retention time=1.56 min; ESI-MS(+) m/z [M+2H]²⁺: 1010.1.

### Preparation of Compound 1778

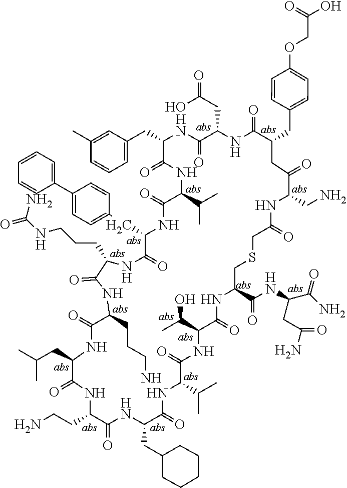 [SMILES: Cc1cccc(C[C@@H]2NC(=O)[C@H](CC(=O)O)NC(=O)[C@H](Cc3ccc(OCC(=O)O)cc3)CC(=O)[C@H](CN)NC(=O)CSC[C@@H](C(=O)N[C@H](CC(N)=O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 1778 was prepared on a 50 μmol scale. The yield of the product was 10 mg, and its estimated purity by LCMS analysis was 100%. Analysis condition A: Retention time=1.54 min; ESI-MS(+) m/z [M+2H]²⁺: 1010.1.

### Preparation of Compound 1779

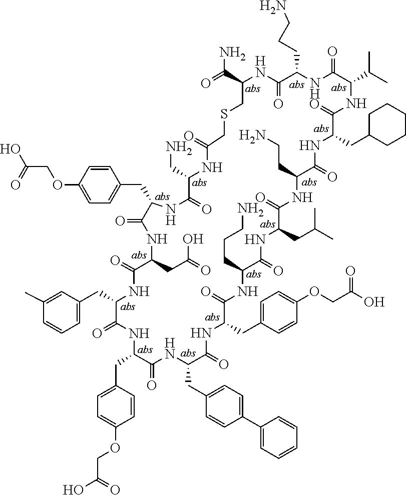 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1779 was prepared on a 50 μmol scale. The yield of the product was 4.5 mg, and its estimated purity by LCMS analysis was 95.1%. Analysis condition B: Retention time=1.47 min; ESI-MS(+) m/z [M+3H]³⁺: 702.2.

### Preparation of Compound 1780

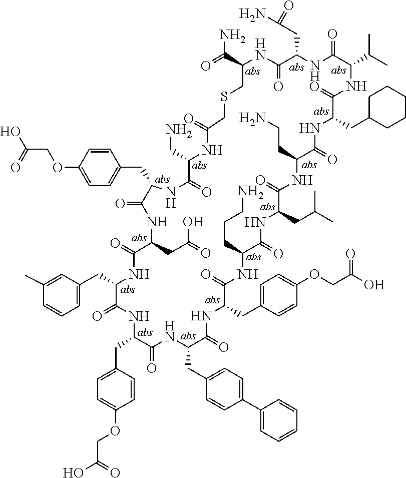 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1780 was prepared on a 50 μmol scale. The yield of the product was 15.1 mg, and its estimated purity by LCMS analysis was 97.7%. Analysis condition: Retention time=1.5 min; ESI-MS(+) m/z [M+2H]²⁺: 1052.

### Preparation of Compound 1781

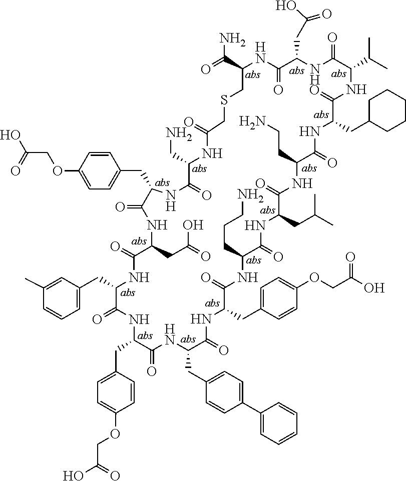 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1781 was prepared on a 50 μmol scale. The yield of the product was 25.2 mg, and its estimated purity by LCMS analysis was 98%. Analysis condition B: Retention time=1.56 min; ESI-MS(+) m/z [M+2H]²⁺: 1053.3.

### Preparation of Compound 1782

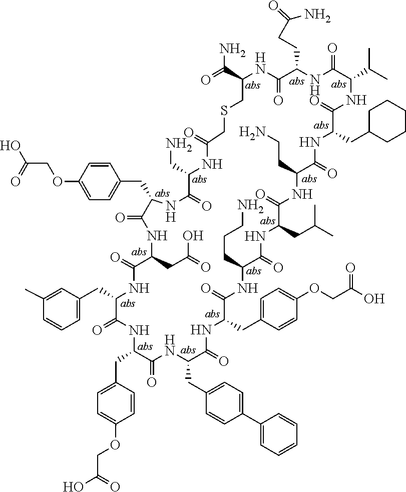 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1782 was prepared on a 50 μmol scale. The yield of the product was 7.7 mg, and its estimated purity by LCMS analysis was 90.5%. Analysis condition B: Retention time=1.55 min; ESI-MS(+) m/z [M+3H]³⁺: 706.4.

### Preparation of Compound 1783

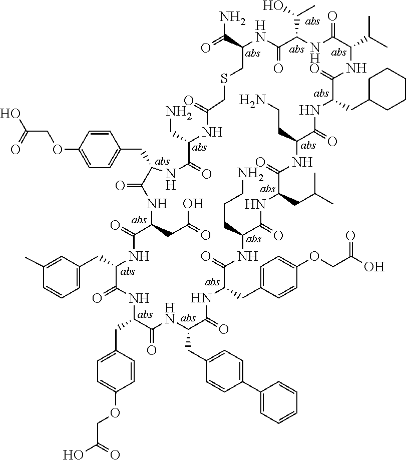 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1783 was prepared on a 50 μmol scale. The yield of the product was 17.8 mg, and its estimated purity by LCMS analysis was 95.1%. Analysis condition A: Retention time=1.51 min; ESI-MS(+) m/z [M+2H]²⁺: 1046.2.

### Preparation of Compound 1784

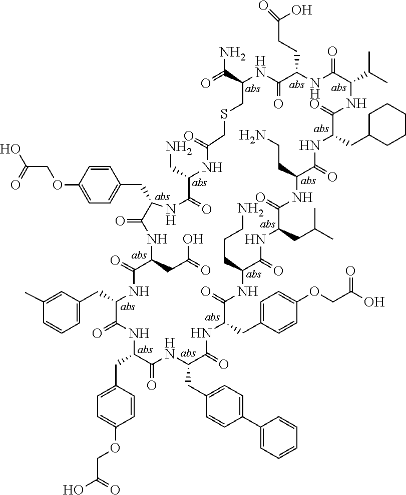 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1784 was prepared on a 50 μmol scale. The yield of the product was 12.9 mg, and its estimated purity by LCMS analysis was 90.4%. Analysis condition B: Retention time=1.56 min; ESI-MS(+) m/z [M+2H]²⁺: 1060.3.

### Preparation of Compound 1785

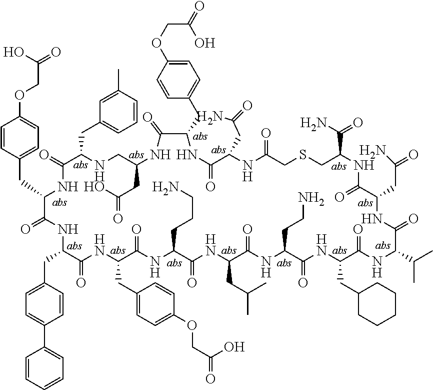 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1785 was prepared on a 50 μmol scale. The yield of the product was 26.7 mg, and its estimated purity by LCMS analysis was 95.8%. Analysis condition: Retention time=1.7 min; ESI-MS(+) m/z [M+2H]²⁺: 1066.2.

### Preparation of Compound 1786

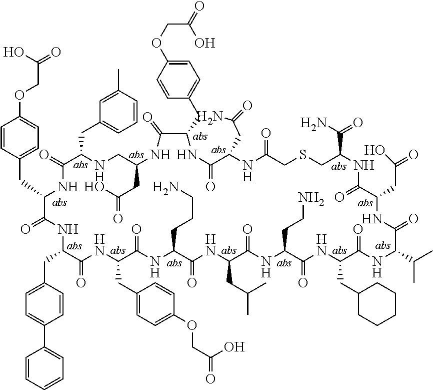 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1786 was prepared on a 50 μmol scale. The yield of the product was 22.3 mg, and its estimated purity by LCMS analysis was 89.9%. Analysis condition B: Retention time=1.61 min; ESI-MS(+) m/z [M+2H]²⁺: 1066.7.

### Preparation of Compound 1787

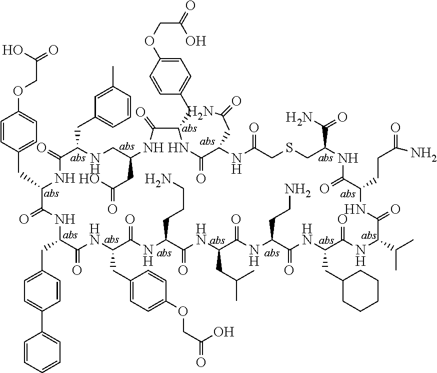 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1787 was prepared on a 50 μmol scale. The yield of the product was 34.7 mg, and its estimated purity by LCMS analysis was 97.7%. Analysis condition A: Retention time=1.45 min; ESI-MS(+) m/z [M+2H]²⁺: 1073.0

### Preparation of Compound 1788

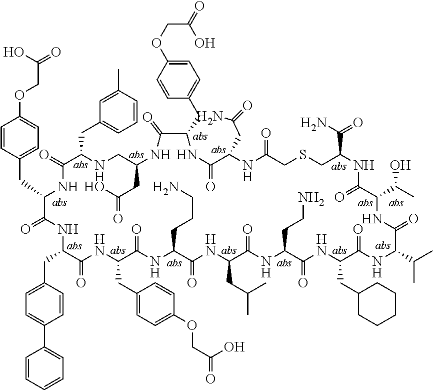 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1788 was prepared on a 50 μmol scale. The yield of the product was 31.3 mg, and its estimated purity by LCMS analysis was 94.6%. Analysis condition B: Retention time=1.65 min; ESI-MS(+) m/z [M+2H]²⁺: 1060.1.

### Preparation of Compound 1789

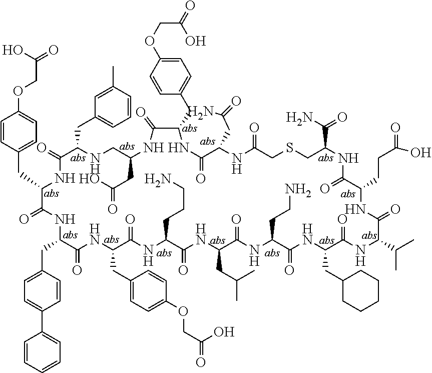 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1789 was prepared on a 50 μmol scale. The yield of the product was 19.9 mg, and its estimated purity by LCMS analysis was 95.1%. Analysis condition A: Retention time=1.62 min; ESI-MS(+) m/z [M+2H]²⁺: 1074.1.

### Preparation of Compound 1790

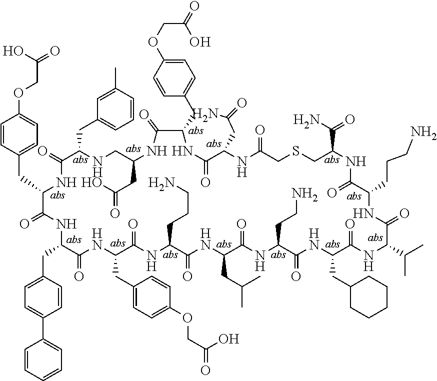 [SMILES: Cc1cccc(C[C@@H]2NC[C@H](CC(=O)O)NC(=O)[C@H](Cc3ccc(OCC(=O)O)cc3)NC(=O)[C@H](CC(N)=O)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1790 was prepared on a 50 μmol scale. The yield of the product was 4.4 mg, and its estimated purity by LCMS analysis was 98%. Analysis condition B: Retention time=1.54 min; ESI-MS(+) m/z [M+2H]²⁺: 1066.3.

### Preparation of Compound 1791

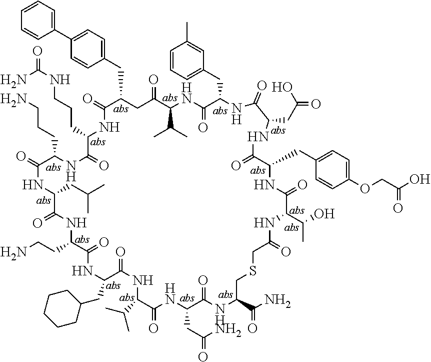 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1791 was prepared on a 50 μmol scale. The yield of the product was 14.3 mg, and its estimated purity by LCMS analysis was 90.9%. Analysis condition B: Retention time=1.68 min; ESI-MS(+) m/z [M+2H]²⁺: 1933.1.

### Preparation of Compound 1792

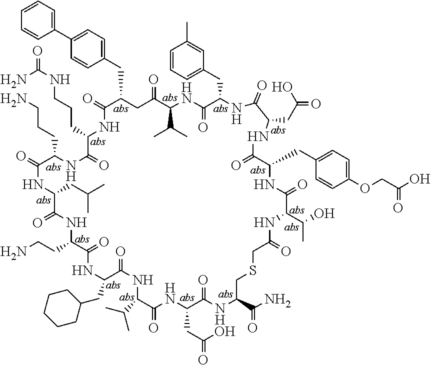 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1792 was prepared on a 50 μmol scale. The yield of the product was 22.2 mg, and its estimated purity by LCMS analysis was 90.9%. Analysis condition B: Retention time=1.69 min; ESI-MS(+) m/z [M+2H]²⁺: 1933.2.

### Preparation of Compound 1793

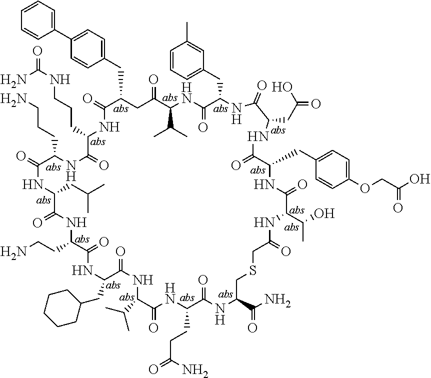 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1793 was prepared on a 50 μmol scale. The yield of the product was 8.6 mg, and its estimated purity by LCMS analysis was 94%. Analysis condition B: Retention time=1.7 min; ESI-MS(+) m/z [M+2H]²⁺: 1945.8.

### Preparation of Compound 1794

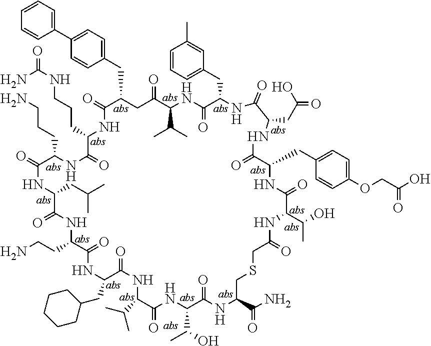 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1794 was prepared on a 50 μmol scale. The yield of the product was 15.8 mg, and its estimated purity by LCMS analysis was 93.9%. Analysis condition B: Retention time=1.69 min; ESI-MS(+) m/z [M+2H]²⁺: 1920.2.

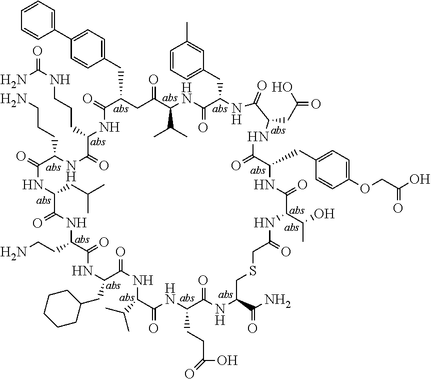 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1795 was prepared on a 50 μmol scale. The yield of the product was 8.2 mg, and its estimated purity by LCMS analysis was 91.7%. Analysis condition B: Retention time=1.56 min; ESI-MS(+) m/z [M+2H]²⁺: 974.

### Preparation of Compound 1796

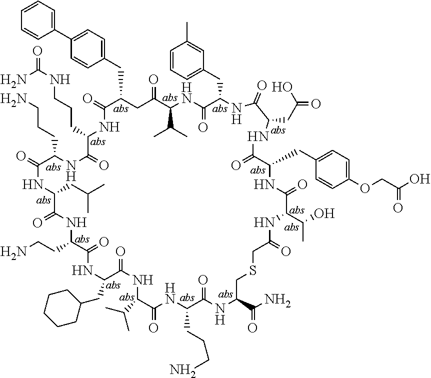 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)CC(=O)[C@H](C(C)C)NC2=O)c1]

Compound 1796 was prepared on a 50 μmol scale. The yield of the product was 12.3 mg, and its estimated purity by LCMS analysis was 93.6%. Analysis condition A: Retention time=1.59 min; ESI-MS(+) m/z [M+H]⁺: 1932.1.

### Preparation of Compound 1797

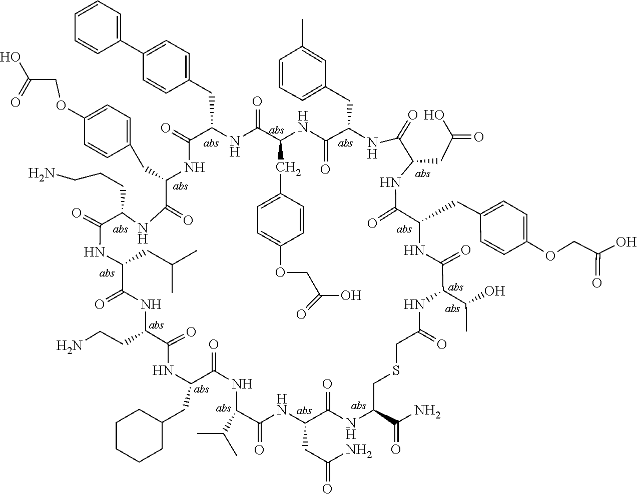 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1797 was prepared on a 50 μmol scale. The yield of the product was 22.4 mg, and its estimated purity by LCMS analysis was 91.3%. Analysis condition B: Retention time=1.68 min; ESI-MS(+) m/z [M+2H]²⁺: 1060.

### Preparation of Compound 1798

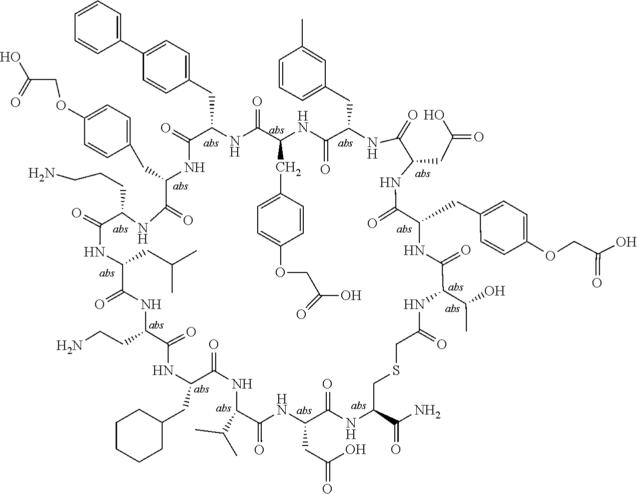 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1798 was prepared on a 50 μmol scale. The yield of the product was 11.5 mg, and its estimated purity by LCMS analysis was 91.7%. Analysis condition A: Retention time=1.39 min; ESI-MS(+) m/z [M+2H]²⁺: 1060.

### Preparation of Compound 1799

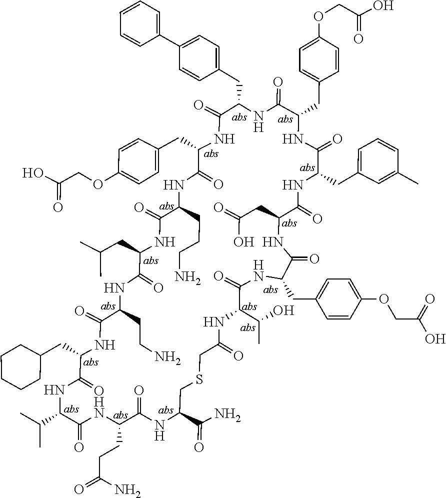 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1799 was prepared on a 50 μmol scale. The yield of the product was 14.9 mg, and its estimated purity by LCMS analysis was 90.3%. Analysis condition B: Retention time=1.64 min; ESI-MS(+) m/z [M+2H]²⁺: 1067.1.

### Preparation of Compound 1800

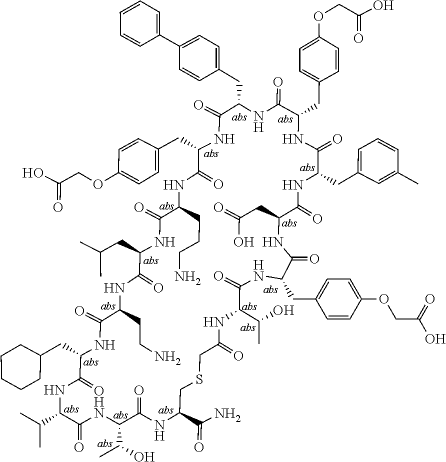 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1800 was prepared on a 50 μmol scale. The yield of the product was 8.4 mg, and its estimated purity by LCMS analysis was 91%. Analysis condition B: Retention time=1.67 min; ESI-MS(+) m/z [M+2H]²⁺: 1053.1.

### Preparation of Compound 1801

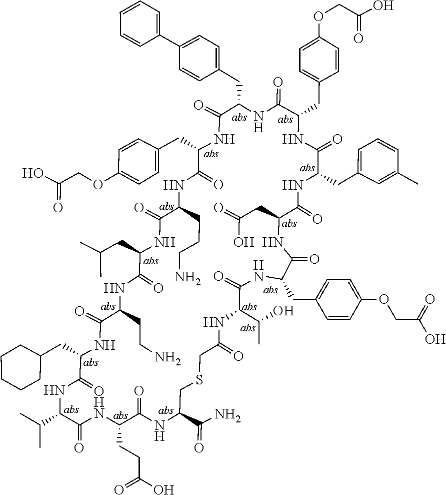 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1801 was prepared on a 50 μmol scale. The yield of the product was 8.5 mg, and its estimated purity by LCMS analysis was 83%. Analysis condition A: Retention time=1.4 min; ESI-MS(+) m/z [M+2H]²⁺: 1067.1.

### Preparation of Compound 1802

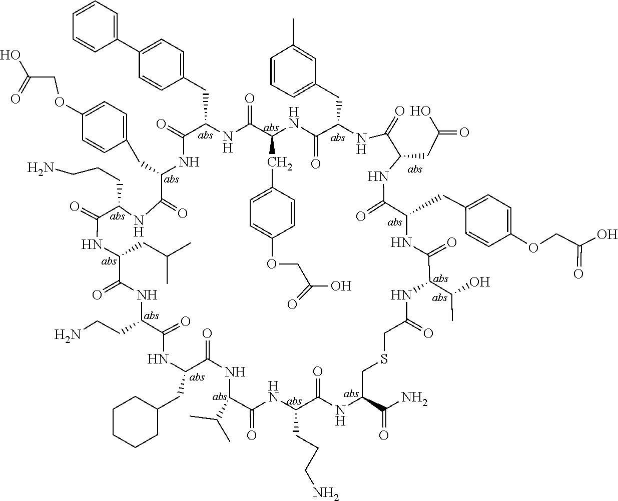 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 1802 was prepared on a 50 μmol scale. The yield of the product was 13 mg, and its estimated purity by LCMS analysis was 95.9%. Analysis condition B: Retention time=1.63 min; ESI-MS(+) m/z [M+2H]²⁺: 1060.

### Preparation of Compound 1803

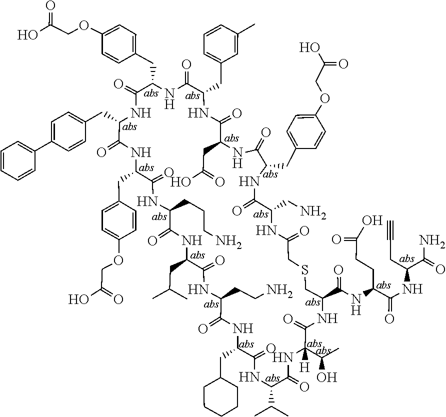 [SMILES: C#CC[C@H](NC(=O)[C@H](CCC(=O)O)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1803 was prepared on a 50 μmol scale. The yield of the product was 3.8 mg, and its estimated purity by LCMS analysis was 100%. Analysis condition B: Retention time=1.57 min; ESI-MS(+) m/z [M+2H]²⁺: 1158.

### Preparation of Compound 1804

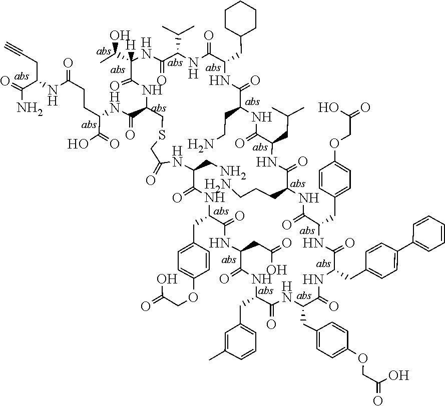 [SMILES: C#CC[C@H](NC(=O)CC[C@H](NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O)C(N)=O]

Compound 1804 was prepared on a 50 μmol scale. The yield of the product was 11.8 mg, and its estimated purity by LCMS analysis was 91.5%. Analysis condition A: Retention time=1.39 min; ESI-MS(+) m/z [M+2H]²⁺: 1158.

### Preparation of Compound 1805

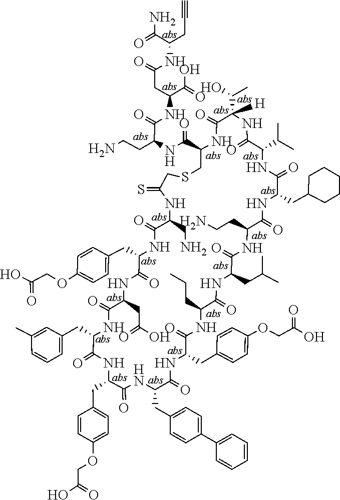 [SMILES: C#CC[C@H](NC(=O)CC[C@@H](CC(=O)O)NC(=O)[C@H](CCN)NC(=O)[C@@H]1CSCC(=S)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCC)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1805 was prepared on a 25 μmol scale. The yield of the product was 4.1 mg, and its estimated purity by LCMS analysis was 100%. Analysis condition A: Retention time=1.36 min; ESI-MS(+) m/z [M+2H]²⁺: 1200.8.

### Preparation of Compound 1806

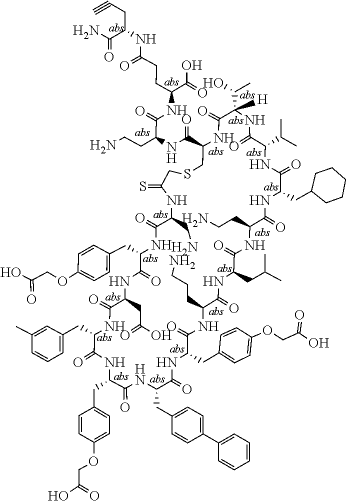 [SMILES: C#CC[C@H](NC(=O)CCC[C@@H](CC(=O)O)NC(=O)[C@H](CCN)NC(=O)[C@@H]1CSCC(=S)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1806 was prepared on a 8.7 μmol scale. The yield of the product was 6.1 mg, and its estimated purity by LCMS analysis was 82.9%. Analysis condition A: Retention time=1.45 min; ESI-MS(+) m/z [M+2H]²⁺: 1208.

### Preparation of Compound 1807

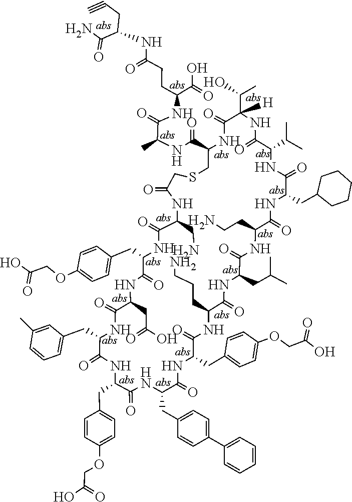 [SMILES: C#CC[C@H](NC(=O)CCC[C@@H](CC(=O)O)NC(=O)[C@H](C)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 1807 was prepared on a 50 μmol scale. The yield of the product was 27.6 mg, and its estimated purity by LCMS analysis was 95%. Analysis condition B: Retention time=1.66 min; ESI-MS(+) m/z [M+3H] 3-: 796.

### Preparation of Compound 1808

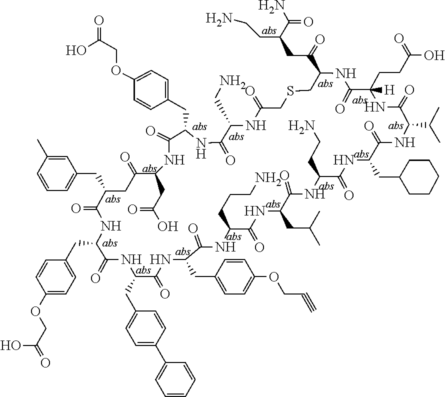 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)CC(=O)[C@H](CC(=O)O)NC(=O)[C@H](Cc3ccc(OCC(=O)O)cc3)NC(=O)[C@H](CN)NC(=O)CSC[C@@H](C(=O)C[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1808 was prepared on a 25 μmol scale. The yield of the product was 5.5 mg, and its estimated purity by LCMS analysis was 89.6%. Analysis condition A: Retention time=1.75 min; ESI-MS(+) m/z [M+2H]²⁺: 1100.

### Preparation of Compound 1809

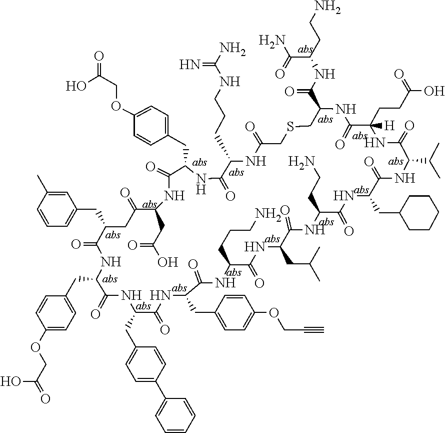 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)CC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1809 was prepared on a 50 μmol scale. The yield of the product was 6.4 mg, and its estimated purity by LCMS analysis was 92%. Analysis condition A: Retention time=1.65 min; ESI-MS(+) m/z [M+2H]²⁺: 1135.1.

### Preparation of Compound 1810

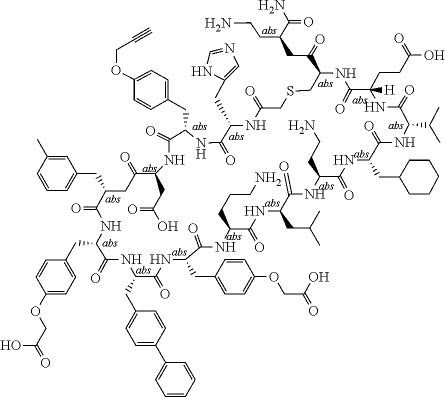 [SMILES: C#CCOc1ccc(C[C@@H]2NC(=O)[C@H](Cc3cnc[nH]3)NC(=O)CSC[C@@H](C(=O)C[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)CC(=O)[C@H](CC(=O)O)NC2=O)cc1]

Compound 1810 was prepared on a 25 μmol scale. The yield of the product was 3.4 mg, and its estimated purity by LCMS analysis was 97.2%. Analysis condition A: Retention time=1.63 min; ESI-MS(+) m/z [M+2H]²⁺: 1125.4.

### Preparation of Compound 1811

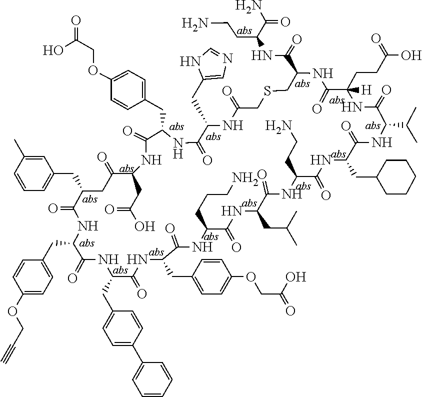 [SMILES: C#CCOc1ccc(C[C@@H]2NC(=O)[C@H](Cc3cccc(C)c3)CC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1811 was prepared on a 25 μmol scale. The yield of the product was 2.9 mg, and its estimated purity by LCMS analysis was 95.1%. Analysis condition B: Retention time=1.63 min; ESI-MS(+) m/z [M+2H]²⁺: 1125.3.

### Preparation of Compound 1812

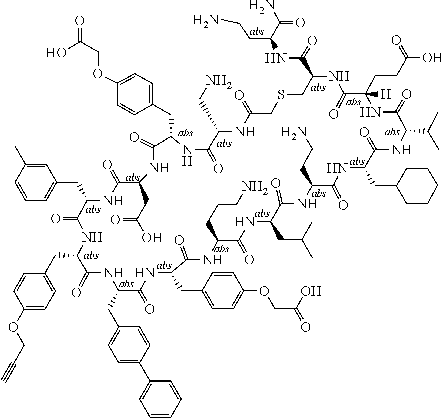 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1812 was prepared on a 25 μmol scale. The yield of the product was 4.2 mg, and its estimated purity by LCMS analysis was 91%. Analysis condition A: Retention time=1.68 min; ESI-MS(+) m/z [M+2H]²⁺: 1100.

### Preparation of Compound 1813

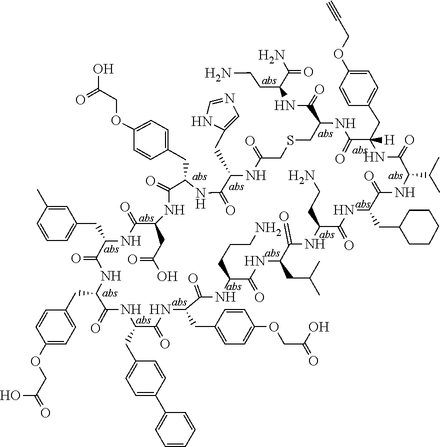 [SMILES: C#CCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@@H](CCN)C(N)=O)NC2=O)cc1]

Compound 1813 was prepared on a 50 μmol scale. The yield of the product was 4.2 mg, and its estimated purity by LCMS analysis was 97.7%. Analysis condition A: Retention time=1.57 min; ESI-MS(+) m/z [M+2H]²⁺: 1171.2.

### Preparation of Compound 1814

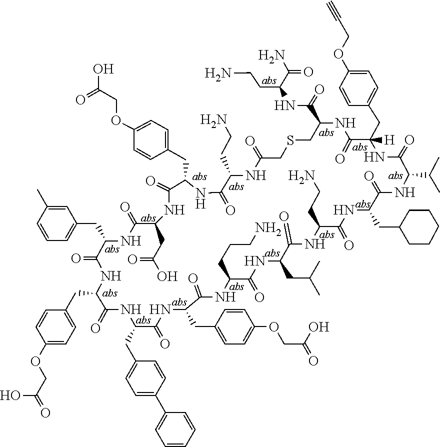 [SMILES: C#CCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC2=O)cc1]

Compound 1814 was prepared on a 25 μmol scale. The yield of the product was 4.8 mg, and its estimated purity by LCMS analysis was 90.3%. Analysis condition B: Retention time=1.63 min; ESI-MS(+) m/z [M+2H]²⁺: 1152.8.

### Preparation of Compound 1815

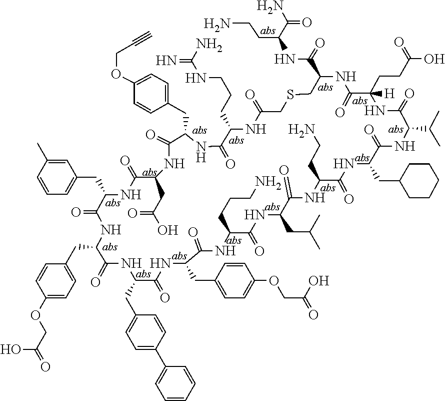 [SMILES: C#CCOc1ccc(C[C@@H]2NC(=O)[C@H](CCCNC(=N)N)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC2=O)cc1]

Compound 1815 was prepared on a 6.6 μmol scale. The yield of the product was 3 mg, and its estimated purity by LCMS analysis was 95.9%. Analysis condition B: Retention time=1.6 min; ESI-MS(+) m/z [M+2H]²⁺: 1135.1.

### Preparation of Compound 1816

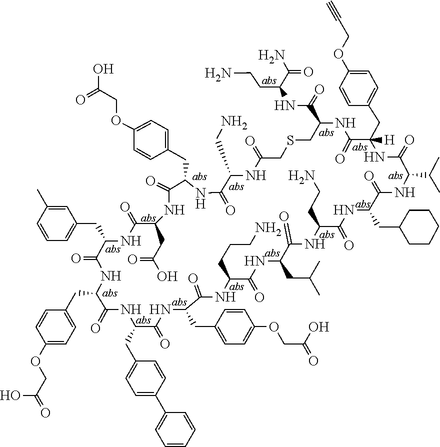 [SMILES: C#CCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC2=O)cc1]

Compound 1816 was prepared on a 50 μmol scale. The yield of the product was 9.7 mg, and its estimated purity by LCMS analysis was 89.5%. Analysis condition B: Retention time=1.56 min; ESI-MS(+) m/z [M+2H]²⁺: 1146.1.

### Preparation of Compound 1817

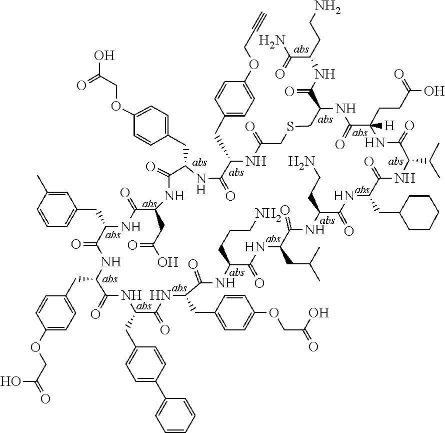 [SMILES: C#CCOc1ccc(C[C@@H]2NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC2=O)cc1]

Compound 1817 was prepared on a 7.7 μmol scale. The yield of the product was 3.9 mg, and its estimated purity by LCMS analysis was 96.4%. Analysis condition B: Retention time=1.77 min; ESI-MS(+) m/z [M+3H]³⁺: 779.

### Preparation of Compound 1818

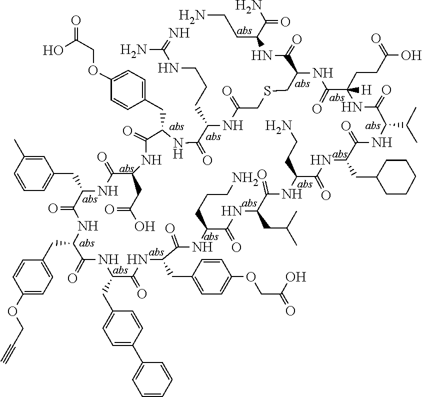 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1818 was prepared on a 25 μmol scale. The yield of the product was 5.6 mg, and its estimated purity by LCMS analysis was 91.5%. Analysis condition A: Retention time=1.65 min; ESI-MS(+) m/z [M+3H]³⁺: 757.2.

### Preparation of Compound 1819

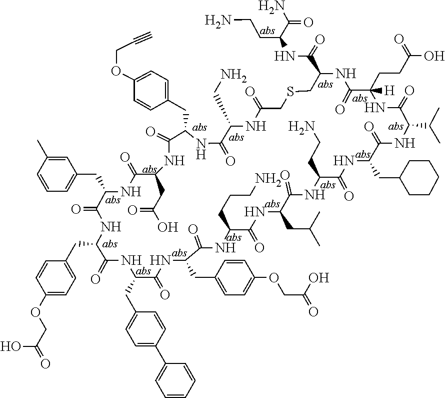 [SMILES: C#CCOc1ccc(C[C@@H]2NC(=O)[C@H](CN)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC2=O)cc1]

Compound 1819 was prepared on a 5.5 μmol scale. The yield of the product was 2.1 mg, and its estimated purity by LCMS analysis was 98%. Analysis condition A: Retention time=1.72 min; ESI-MS(+) m/z [M+2H]²⁺: 1100.

### Preparation of Compound 1820

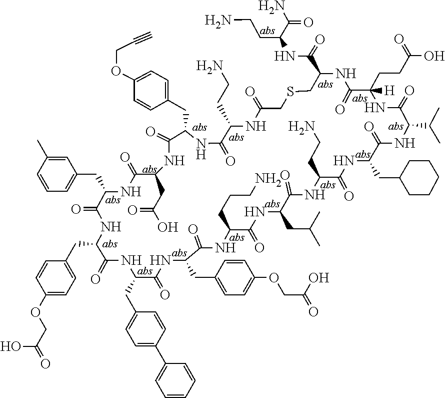 [SMILES: C#CCOc1ccc(C[C@@H]2NC(=O)[C@H](CCN)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC2=O)cc1]

Compound 1820 was prepared on a 25 μmol scale. The yield of the product was 2.3 mg, and its estimated purity by LCMS analysis was 95.5%. Analysis condition A: Retention time=1.54 min; ESI-MS(+) m/z [M+2H]²⁺: 1106.9.

### Preparation of Compound 1821

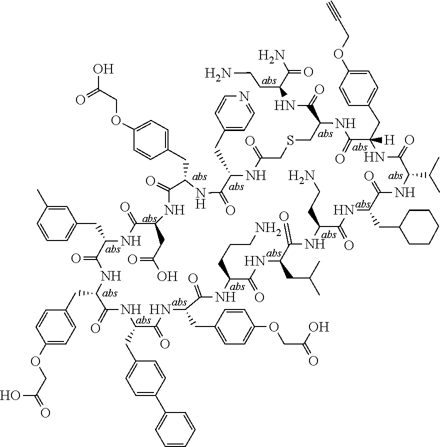 [SMILES: C#CCOc1ccc(C[C@@H]2NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC2=O)cc1]

Compound 1821 was prepared on a 3.8 μmol scale. The yield of the product was 3 mg, and its estimated purity by LCMS analysis was 100%. Analysis condition A: Retention time=1.69 min; ESI-MS(+) m/z [M+2H]²⁺: 1176.9.

### Preparation of Compound 1822

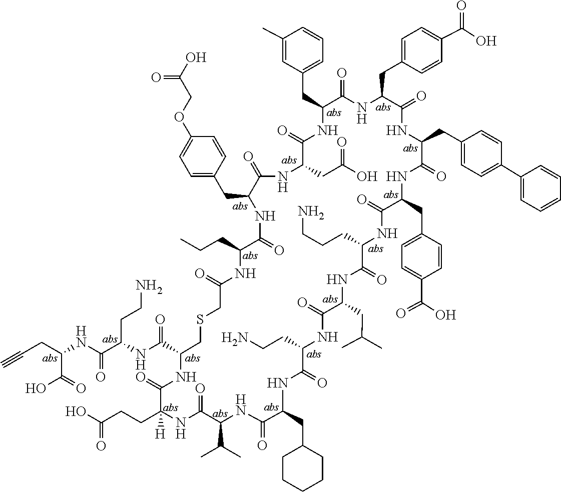 [SMILES: C#CC[C@H](NC(=O)[C@H](CCN)NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1822 was prepared on a 25 μmol scale. The yield of the product was 2.6 mg, and its estimated purity by LCMS analysis was 92.8%. Analysis condition A: Retention time=1.33 min; ESI-MS(+) m/z [M+2H]²⁺: 1134.2.

### Preparation of Compound 1823

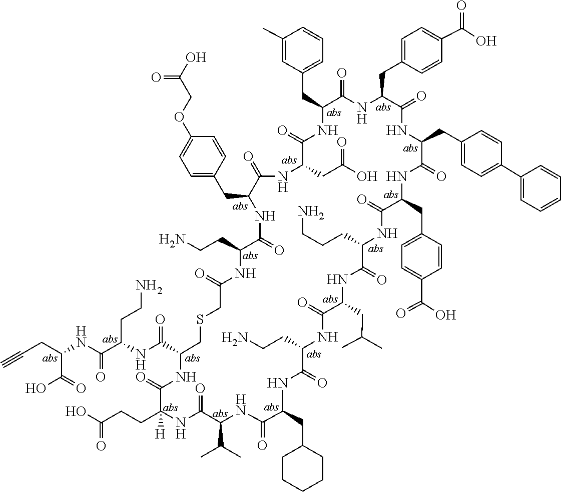 [SMILES: C#CC[C@H](NC(=O)[C@H](CCN)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1823 was prepared on a 2.6 μmol scale. The yield of the product was 0.6 mg, and its estimated purity by LCMS analysis was 95.1%. Analysis condition B: Retention time=1.57 min; ESI-MS(+) m/z [M+2H]²⁺: 1134.8.

### Preparation of Compound 1824

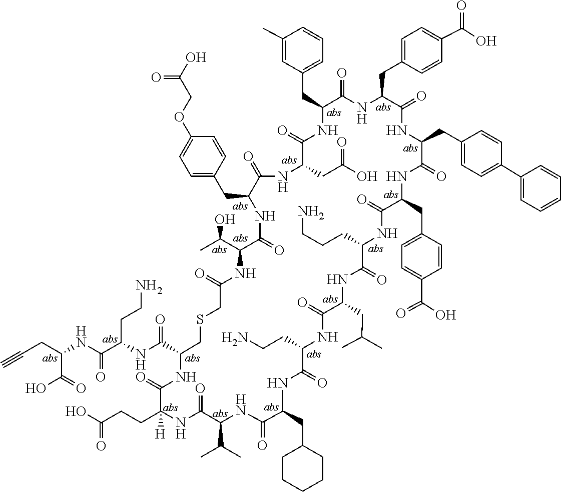 [SMILES: C#CC[C@H](NC(=O)[C@H](CCN)NC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1824 was prepared on a 3.5 μmol scale. The yield of the product was 3.1 mg, and its estimated purity by LCMS analysis was 91.1%. Analysis condition A: Retention time=1.34 min; ESI-MS(+) m/z [M+2H]²⁺: 1136.1.

### Preparation of Compound 1825

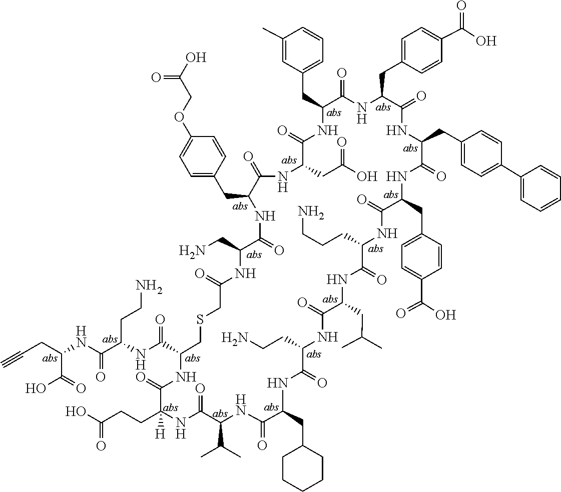 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1825 was prepared on a 2.2 μmol scale. The yield of the product was 1.3 mg, and its estimated purity by LCMS analysis was 83%. Analysis condition A: Retention time=1.48 min; ESI-MS(+) m/z [M+3H]³⁺: 752.4.

### Preparation of Compound 1826

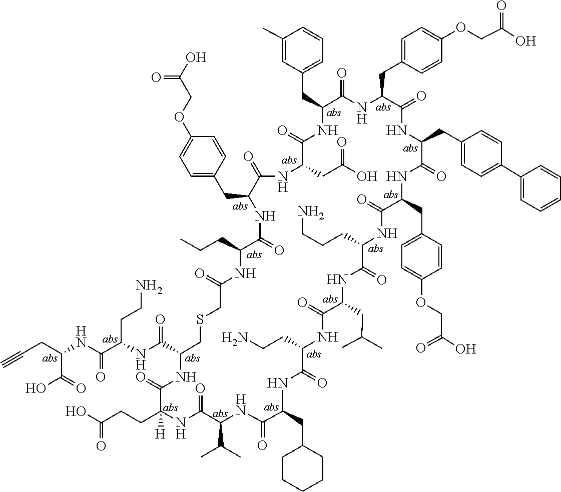 [SMILES: C#CC[C@H](NC(=O)[C@H](CCN)NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1826 was prepared on a 50 μmol scale. The yield of the product was 6 mg, and its estimated purity by LCMS analysis was 94%. Analysis condition A: Retention time=1.34 min; ESI-MS(+) m/z [M+2H]²⁺: 1164.2.

### Preparation of Compound 1827

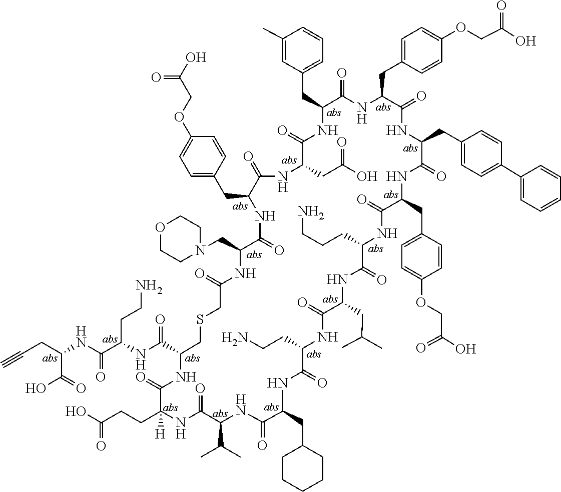 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 1827 was prepared on a 50 μmol scale. The yield of the product was 2.9 mg, and its estimated purity by LCMS analysis was 96.5%. Analysis condition A: Retention time=1.36 min; ESI-MS(+) m/z [M+2H]²⁺: 1192.7.

### Preparation of Compound 1828

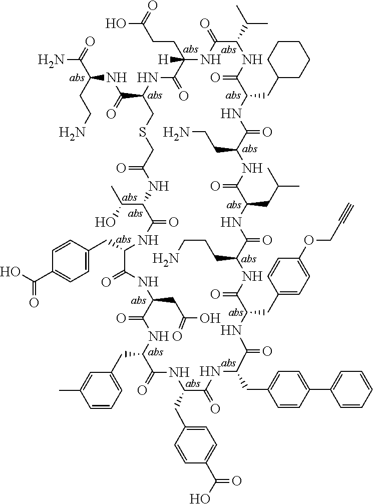 [SMILES: C#CCOc1ccc(C[C@@H]2NC(=O)[C@H](Cc3ccc(-c4ccccc4)cc3)NC(=O)[C@H](Cc3ccc(C(=O)O)cc3)NC(=O)[C@H](Cc3cccc(C)c3)NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1828 was prepared on a 25 μmol scale. The yield of the product was 3.4 mg, and its estimated purity by LCMS analysis was 96%. Analysis condition A: Retention time=1.69 min; ESI-MS(+) m/z [M+2H]²⁺: 1077.1.

### Preparation of Compound 1829

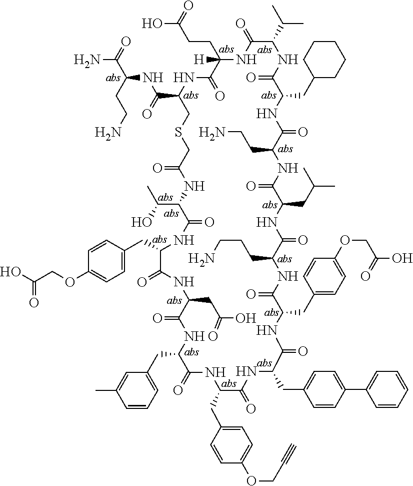 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1829 was prepared on a 25 μmol scale. The yield of the product was 4.2 mg, and its estimated purity by LCMS analysis was 88%. Analysis condition A: Retention time=1.63 min; ESI-MS(+) m/z [M+2H]²⁺: 1107.1.

### Preparation of Compound 1830

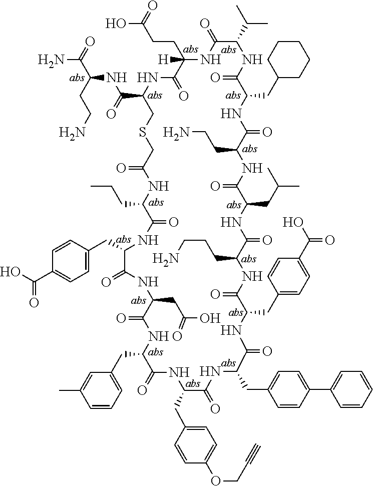 [SMILES: C#CCOc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H](CCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Compound 1830 was prepared on a 25 μmol scale. The yield of the product was 9.8 mg, and its estimated purity by LCMS analysis was 95.9%. Analysis condition B: Retention time=1.72 min; ESI-MS(+) m/z [M+2H]²⁺: 1076.2.

### Preparation of Compound 1831

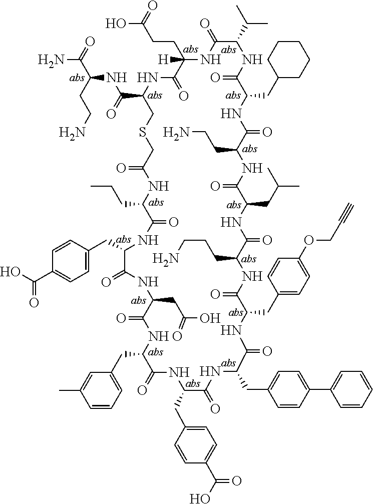 [SMILES: C#CCOc1ccc(C[C@@H]2NC(=O)[C@H](Cc3ccc(-c4ccccc4)cc3)NC(=O)[C@H](Cc3ccc(C(=O)O)cc3)NC(=O)[C@H](Cc3cccc(C)c3)NC(=O)[C@H](CC(=O)O)NC(=O)[C@H](Cc3ccc(C(=O)O)cc3)NC(=O)[C@H](CCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1831 was prepared on a 25 μmol scale. The yield of the product was 11.3 mg, and its estimated purity by LCMS analysis was 86.6%. Analysis condition A: Retention time=1.66 min; ESI-MS(+) m/z [M+2H]²⁺: 1076.3.

### Preparation of Compound 1832

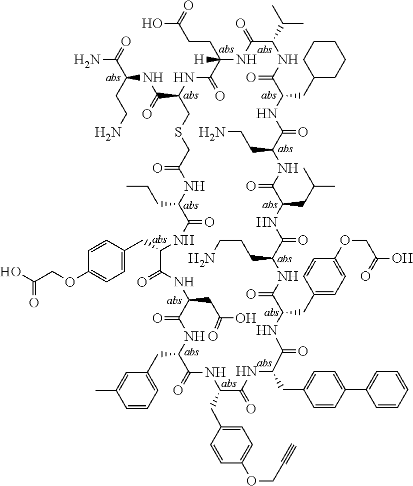 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1832 was prepared on a 25 μmol scale. The yield of the product was 8.4 mg, and its estimated purity by LCMS analysis was 82.4%. Analysis condition A: Retention time=1.7 min; ESI-MS(+) m/z [M+3H]³⁺: 738.1.

### Preparation of Compound 1833

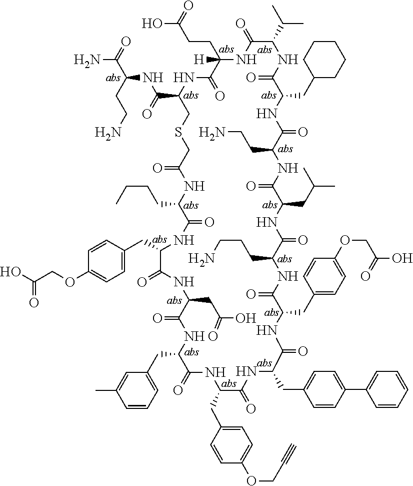 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CCCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1833 was prepared on a 25 μmol scale. The yield of the product was 5.9 mg, and its estimated purity by LCMS analysis was 95.4%. Analysis condition B: Retention time=1.69 min; ESI-MS(+) m/z [M+2H]²⁺: 1113.4.

### Preparation of Compound 1834

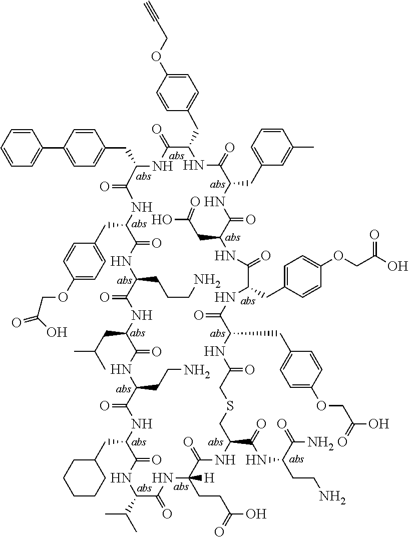 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](Cc3ccc(OCC(=O)O)cc3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1834 was prepared on a 25 μmol scale. The yield of the product was 9.1 mg, and its estimated purity by LCMS analysis was 89.6%. Analysis condition A: Retention time=1.57 min; ESI-MS(+) m/z [M+3H] 3-: 778.5.

### Preparation of Compound 1835

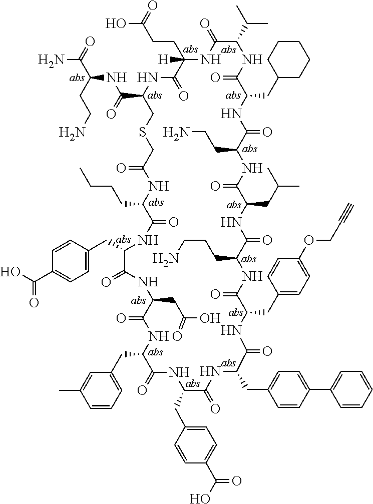 [SMILES: C#CCOc1ccc(C[C@@H]2NC(=O)[C@H](Cc3ccc(-c4ccccc4)cc3)NC(=O)[C@H](Cc3ccc(C(=O)O)cc3)NC(=O)[C@H](Cc3cccc(C)c3)NC(=O)[C@H](CC(=O)O)NC(=O)[C@H](Cc3ccc(C(=O)O)cc3)NC(=O)[C@H](CCCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1835 was prepared on a 50 μmol scale. The yield of the product was 14.6 mg, and its estimated purity by LCMS analysis was 91.1%. Analysis condition A: Retention time=1.71 min; ESI-MS(+) m/z [M+2H]²⁺: 1083.2.

### Preparation of Compound 1836

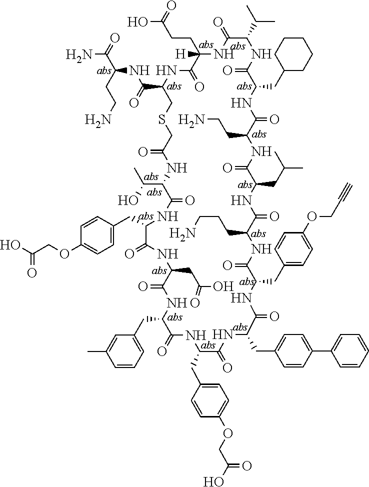 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1836 was prepared on a 25 μmol scale. The yield of the product was 4.7 mg, and its estimated purity by LCMS analysis was 99.2%. Analysis condition B: Retention time=1.7 min; ESI-MS(+) m/z [M+2H]²⁺: 1107.3.

### Preparation of Compound 1837

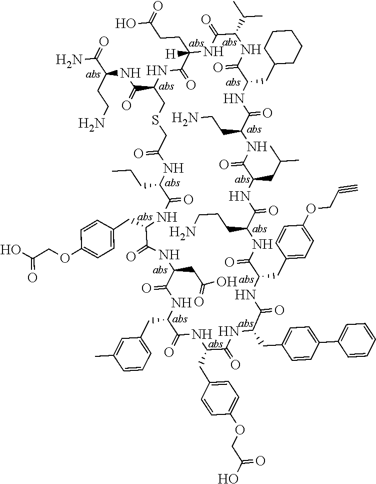 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1837 was prepared on a 50 μmol scale. The yield of the product was 8.8 mg, and its estimated purity by LCMS analysis was 87.6%. Analysis condition A: Retention time=1.65 min; ESI-MS(+) m/z [M+2H]²⁺: 1106.4.

### Preparation of Compound 1838

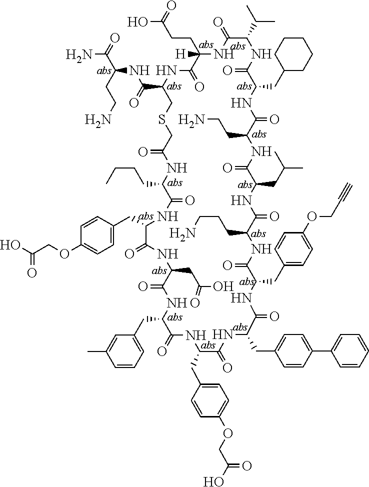 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CCCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1838 was prepared on a 25 μmol scale. The yield of the product was 2.6 mg, and its estimated purity by LCMS analysis was 88.5%. Analysis condition A: Retention time=1.66 min; ESI-MS(+) m/z [M+2H]²⁺: 1113.2.

### Preparation of Compound 1839

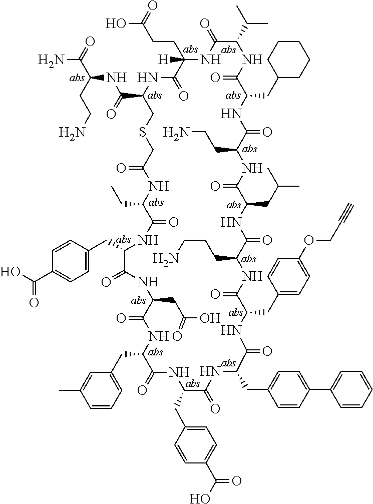 [SMILES: C#CCOc1ccc(C[C@@H]2NC(=O)[C@H](Cc3ccc(-c4ccccc4)cc3)NC(=O)[C@H](Cc3ccc(C(=O)O)cc3)NC(=O)[C@H](Cc3cccc(C)c3)NC(=O)[C@H](CC(=O)O)NC(=O)[C@H](Cc3ccc(C(=O)O)cc3)NC(=O)[C@H](CC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1839 was prepared on a 25 μmol scale. The yield of the product was 8.6 mg, and its estimated purity by LCMS analysis was 95.1%. Analysis condition A: Retention time=1.71 min; ESI-MS(+) m/z [M+2H]²⁺: 1069.4.

### Preparation of Compound 1840

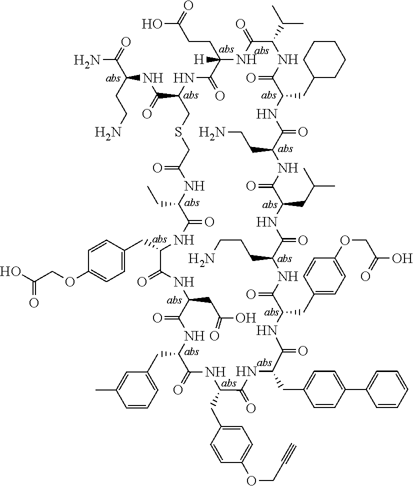 [SMILES: C#CCOc1ccc(C[C@@H]2NC(=O)[C@H](Cc3cccc(C)c3)NC(=O)[C@H](CC(=O)O)NC(=O)[C@H](Cc3ccc(OCC(=O)O)cc3)NC(=O)[C@H](CC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC2=O)cc1]

Compound 1840 was prepared on a 25 μmol scale. The yield of the product was 7.1 mg, and its estimated purity by LCMS analysis was 93%. Analysis condition A: Retention time=1.59 min; ESI-MS(+) m/z [M+2H]²⁺: 1099.4.

### Preparation of Compound 1841

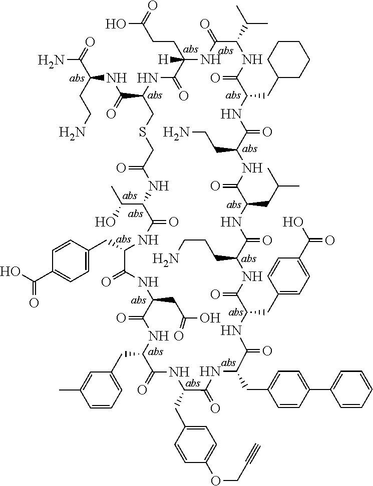 [SMILES: C#CCOc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H]([C@@H](C)O)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Compound 1841 was prepared on a 25 μmol scale. The yield of the product was 5.5 mg, and its estimated purity by LCMS analysis was 92.5%. Analysis condition A: Retention time=1.54 min; ESI-MS(+) m/z [M+2H]²⁺: 1077.2.

### Preparation of Compound 1842

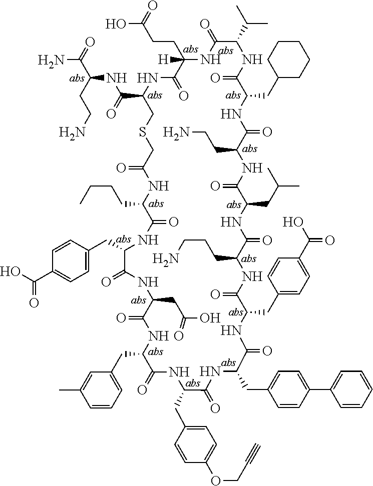 [SMILES: C#CCOc1ccc(C[C@@H]2NC(=O)[C@H](Cc3cccc(C)c3)NC(=O)[C@H](CC(=O)O)NC(=O)[C@H](Cc3ccc(C(=O)O)cc3)NC(=O)[C@H](CCCC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Compound 1842 was prepared on a 25 μmol scale. The yield of the product was 7.9 mg, and its estimated purity by LCMS analysis was 91.7%. Analysis condition A: Retention time=1.72 min; ESI-MS(+) m/z [M+3H]³⁺: 722.4.

### Preparation of Compound 1843

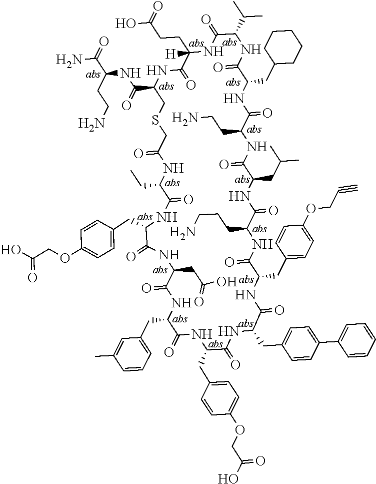 [SMILES: C#CCOc1ccc(C[C@@H]2NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC(=O)[C@H](CC)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1843 was prepared on a 25 μmol scale. The yield of the product was 3.7 mg, and its estimated purity by LCMS analysis was 95.9%. Analysis condition A: Retention time=1.62 min; ESI-MS(+) m/z [M+2H]²⁺: 1099.3.

### Preparation of Compound 1844

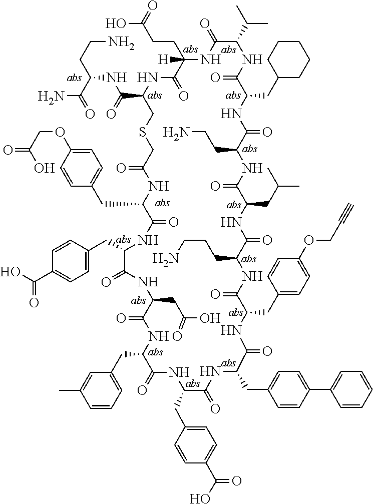 [SMILES: C#CCOc1ccc(C[C@@H]2NC(=O)[C@H](Cc3ccc(-c4ccccc4)cc3)NC(=O)[C@H](Cc3ccc(C(=O)O)cc3)NC(=O)[C@H](Cc3cccc(C)c3)NC(=O)[C@H](CC(=O)O)NC(=O)[C@H](Cc3ccc(C(=O)O)cc3)NC(=O)[C@H](Cc3ccc(OCC(=O)O)cc3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC2=O)cc1]

Compound 1844 was prepared on a 50 μmol scale. The yield of the product was 8.9 mg, and its estimated purity by LCMS analysis was 94.8%. Analysis condition A: Retention time=1.56 min; ESI-MS(+) m/z [M+2H]²⁺: 1137.1.

### Preparation of Compound 1845

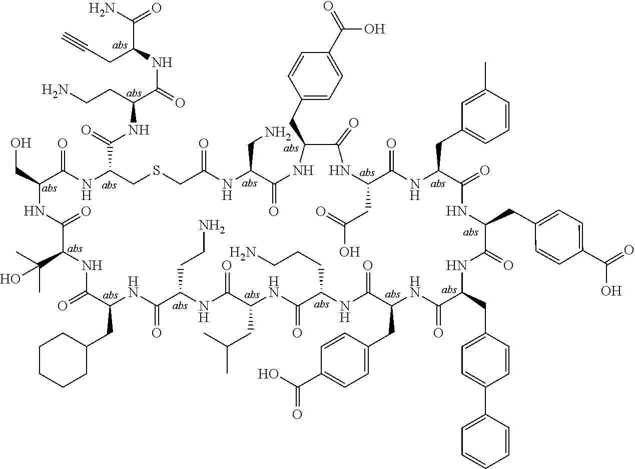 [SMILES: C#CC[C@H](NC(=O)[C@H](CCN)NC(=O)[C@@H]1CSCC(=O)N[C@@H](CN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1845 was prepared on a 25 μmol scale. The yield of the product was 3.9 mg, and its estimated purity by LCMS analysis was 91.8%. Analysis condition B: Retention time=1.52 min; ESI-MS(+) m/z [M+3H]³⁺: 733.3.

### Preparation of Compound 1846

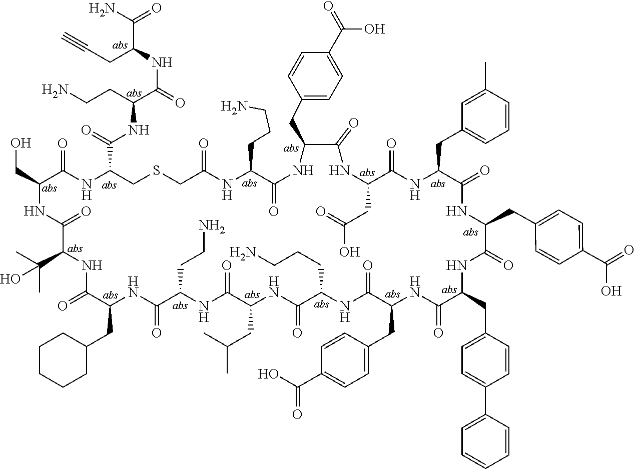 [SMILES: C#CC[C@H](NC(=O)[C@H](CCN)NC(=O)[C@@H]1CSCC(=O)N[C@@H](CCCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1846 was prepared on a 2.2 μmol scale. The yield of the product was 0.9 mg, and its estimated purity by LCMS analysis was 100%. Analysis condition A: Retention time=1.44 min; ESI-MS(+) m/z [M+2H]²⁺: 1113.1.

### Preparation of Compound 1847

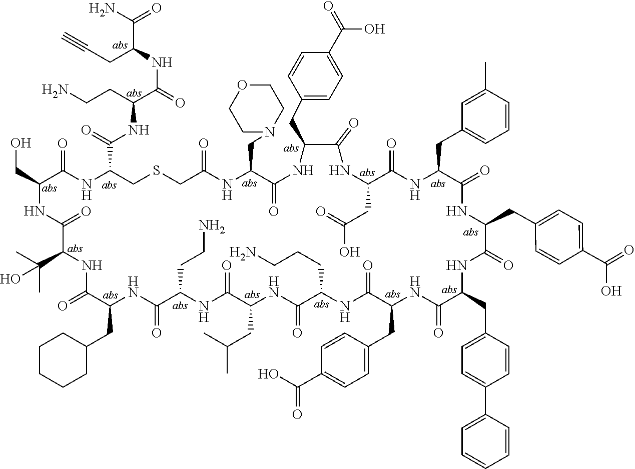 [SMILES: C#CC[C@H](NC(=O)[C@H](CCN)NC(=O)[C@@H]1CSCC(=O)N[C@@H](CN2CCOCC2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1847 was prepared on a 25 μmol scale. The yield of the product was 2.3 mg, and its estimated purity by LCMS analysis was 96.6%. Analysis condition A: Retention time=1.43 min; ESI-MS(+) m/z [M+2H]²⁺: 1134.3.

### Preparation of Compound 1848

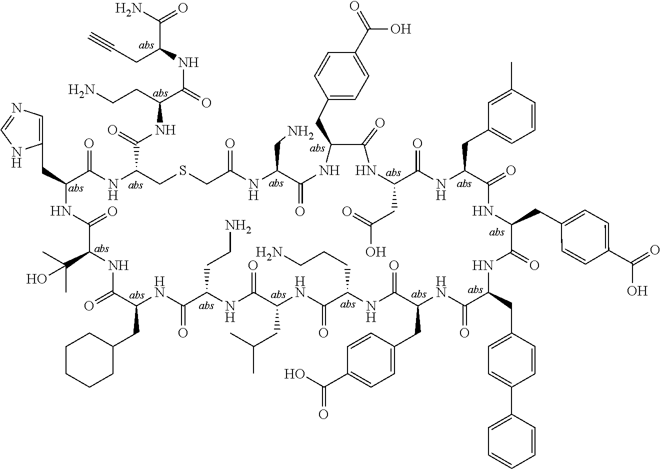 [SMILES: C#CC[C@H](NC(=O)[C@H](CCN)NC(=O)[C@@H]1CSCC(=O)N[C@@H](CN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](Cc2cnc[nH]2)C(=O)N1)C(N)=O]

Compound 1848 was prepared on a 25 μmol scale. The yield of the product was

10.7 mg, and its estimated purity by LCMS analysis was 96.3%. Analysis condition B: Retention time=1.49 min; ESI-MS(+) m/z [M+3H]³⁺: 750.1.

### Preparation of Compound 1849

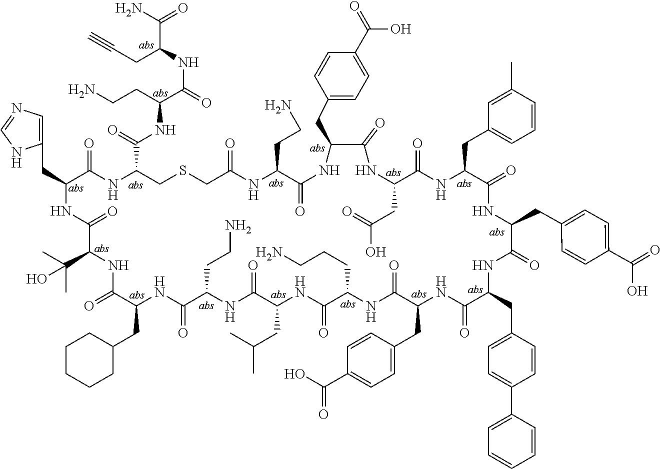 [SMILES: C#CC[C@H](NC(=O)[C@H](CCN)NC(=O)[C@@H]1CSCC(=O)N[C@@H](CCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](Cc2cnc[nH]2)C(=O)N1)C(N)=O]

Compound 1849 was prepared on a 50 μmol scale. The yield of the product was 16.9 mg, and its estimated purity by LCMS analysis was 90.5%. Analysis condition A: Retention time=1.57 min; ESI-MS(+) m/z [M+3H]³⁺: 755.

### Preparation of Compound 1850

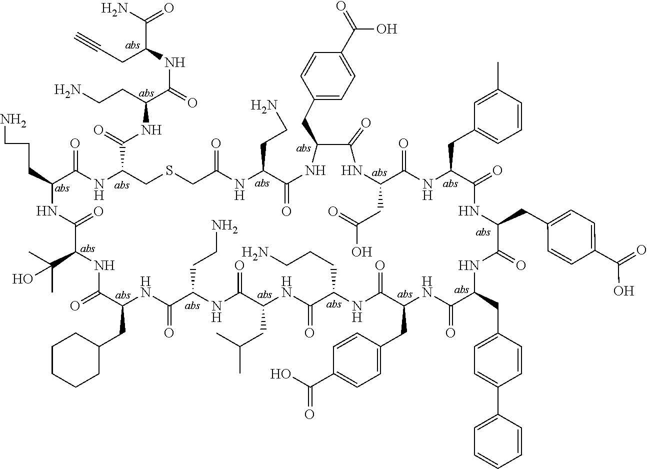 [SMILES: C#CC[C@H](NC(=O)[C@H](CCN)NC(=O)[C@@H]1CSCC(=O)N[C@@H](CCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCN)C(=O)N1)C(N)=O]

Compound 1850 was prepared on a 50 μmol scale. The yield of the product was 13.1 mg, and its estimated purity by LCMS analysis was 96.6%. Analysis condition B: Retention time=1.47 min; ESI-MS(+) m/z [M+3H]³⁺: 747.2.

### Preparation of Compound 1851

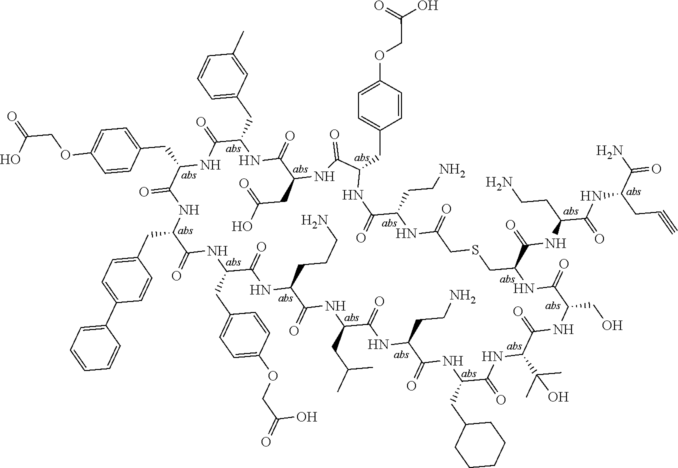 [SMILES: C#CC[C@H](NC(=O)[C@H](CCN)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1851 was prepared on a 25 μmol scale. The yield of the product was 3.2 mg, and its estimated purity by LCMS analysis was 97.4%. Analysis condition A: Retention time=1.48 min; ESI-MS(+) m/z [M+3H]³⁺: 768.

### Preparation of Compound 1852

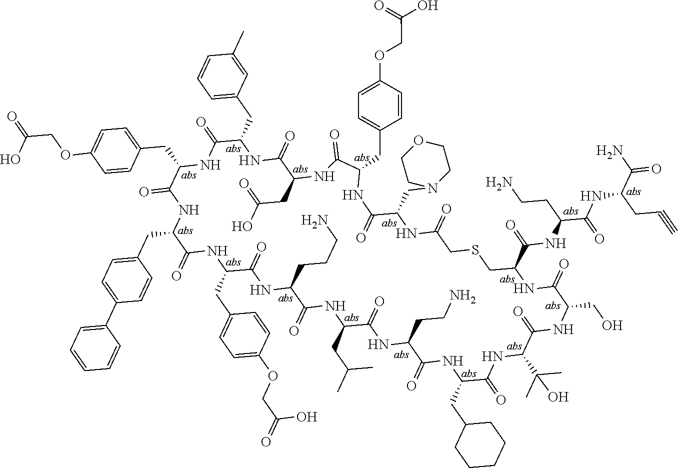 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1852 was prepared on a 25 μmol scale. The yield of the product was 1 mg, and its estimated purity by LCMS analysis was 96.9%. Analysis condition B: Retention time=1.54 min; ESI-MS(+) m/z [M+2H]²⁺: 1179.3.

### Preparation of Compound 1853

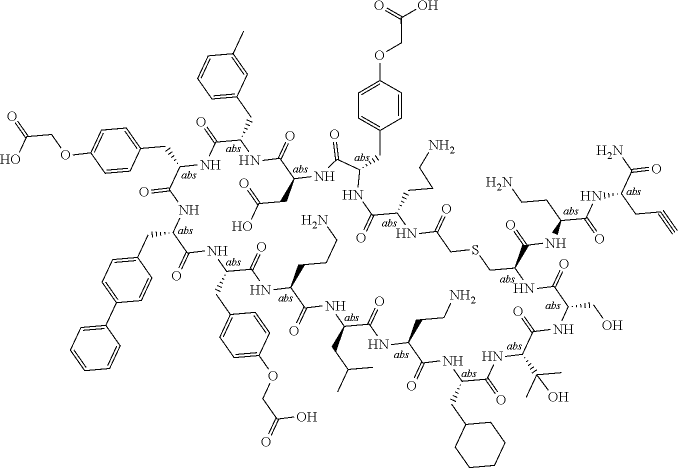 [SMILES: C#CC[C@H](NC(=O)[C@H](CCN)NC(=O)[C@@H]1CSCC(=O)N[C@@H](CCCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1853 was prepared on a 50 μmol scale. The yield of the product was 11.2 mg, and its estimated purity by LCMS analysis was 100%. Analysis condition B: Retention time=1.56 min; ESI-MS(+) m/z [M+2H]²⁺: 1158.2.

### Preparation of Compound 1854

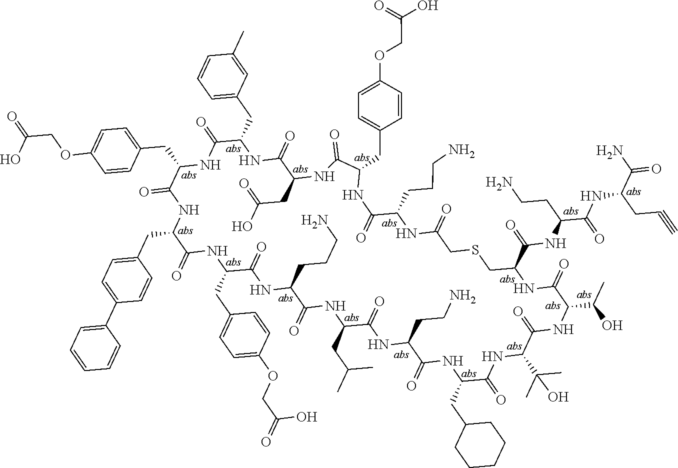 [SMILES: C#CC[C@H](NC(=O)[C@H](CCN)NC(=O)[C@@H]1CSCC(=O)N[C@@H](CCCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H]([C@@H](C)O)C(=O)N1)C(N)=O]

Compound 1854 was prepared on a 25 μmol scale. The yield of the product was 18.7 mg, and its estimated purity by LCMS analysis was 86.2%. Analysis condition A: Retention time=1.53 min; ESI-MS(+) m/z [M+2H]²⁺: 1165.4.

### Preparation of Compound 1855

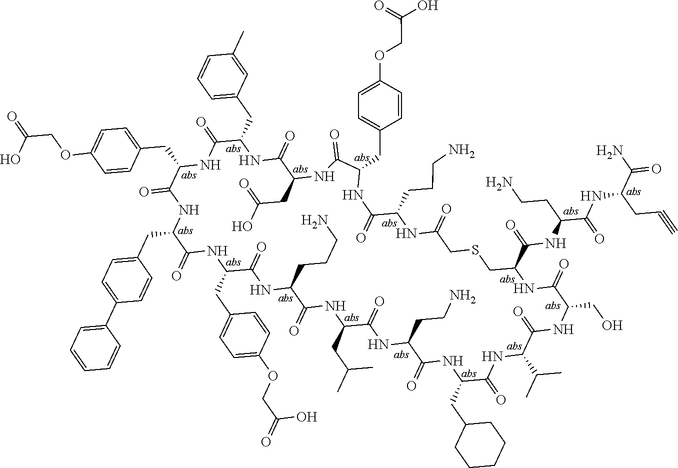 [SMILES: C#CC[C@H](NC(=O)[C@H](CCN)NC(=O)[C@@H]1CSCC(=O)N[C@@H](CCCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1)C(N)=O]

Compound 1855 was prepared on a 25 μmol scale. The yield of the product was 3.5 mg, and its estimated purity by LCMS analysis was 93.5%. Analysis condition A: Retention time=1.57 min; ESI-MS(+) m/z [M+2H]²⁺: 1150.1.

### Preparation of Compound 1856

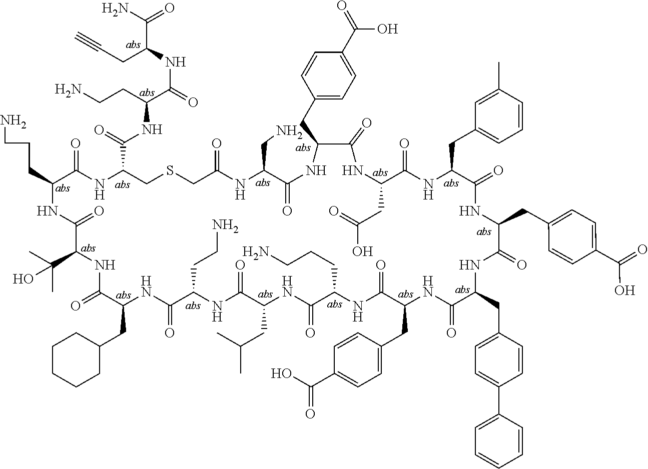 [SMILES: C#CC[C@H](NC(=O)[C@H](CCN)NC(=O)[C@@H]1CSCC(=O)N[C@@H](CN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCN)C(=O)N1)C(N)=O]

Compound 1856 was prepared on a 50 μmol scale. The yield of the product was 12.1 mg, and its estimated purity by LCMS analysis was 97%. Analysis condition A: Retention time=1.55 min; ESI-MS(+) m/z [M+2H]²⁺: 1113.

### Preparation of Compound 1857

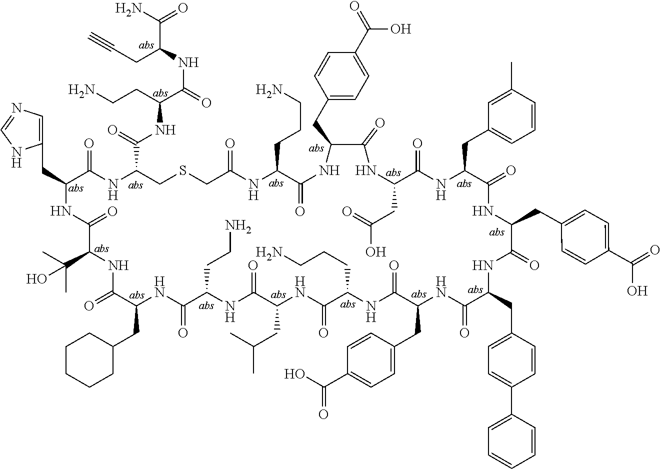 [SMILES: C#CC[C@H](NC(=O)[C@H](CCN)NC(=O)[C@@H]1CSCC(=O)N[C@@H](CCCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](Cc2cnc[nH]2)C(=O)N1)C(N)=O]

Compound 1857 was prepared on a 13 μmol scale. The yield of the product was 2.7 mg, and its estimated purity by LCMS analysis was 98.7%. Analysis condition B: Retention time=1.41 min; ESI-MS(+) m/z [M+2H]²⁺: 1138.3.

### Preparation of Compound 1858

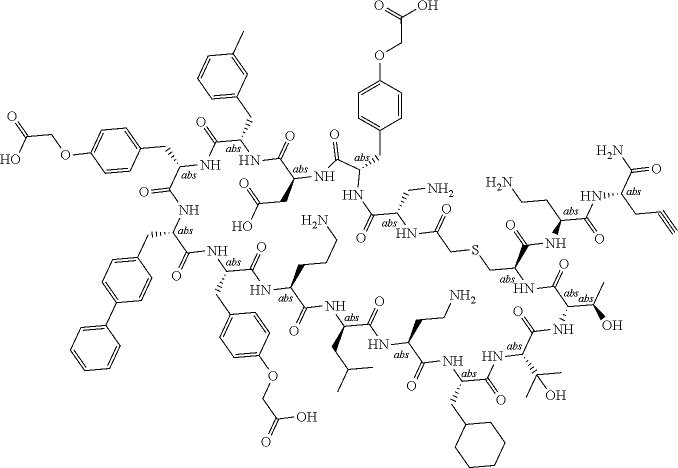 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H]([C@@H](C)O)C(=O)N1)C(N)=O]

Compound 1858 was prepared on a 50 μmol scale. The yield of the product was 19.2 mg, and its estimated purity by LCMS analysis was 98.3%. Analysis condition A: Retention time=1.62 min; ESI-MS(+) m/z [M+2H]²⁺: 1151.3.

### Preparation of Compound 1859

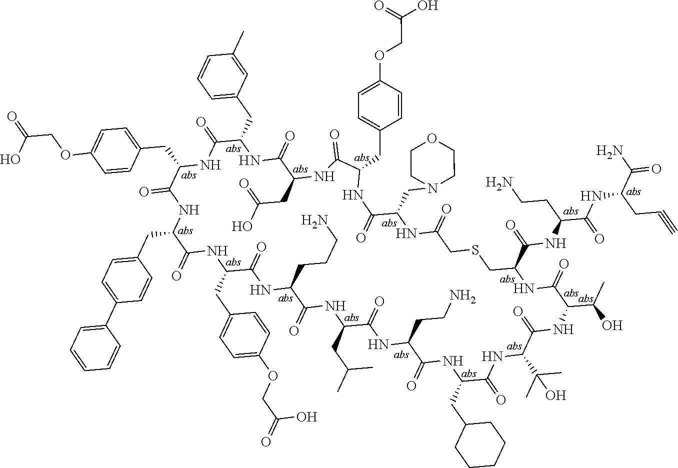 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H]([C@@H](C)O)C(=O)N1)C(N)=O]

Compound 1859 was prepared on a 50 μmol scale. The yield of the product was 16.5 mg, and its estimated purity by LCMS analysis was 96.3%. Analysis condition A: Retention time=1.6 min; ESI-MS(+) m/z [M+3H]³⁺: 791.2.

### Preparation of Compound 1860

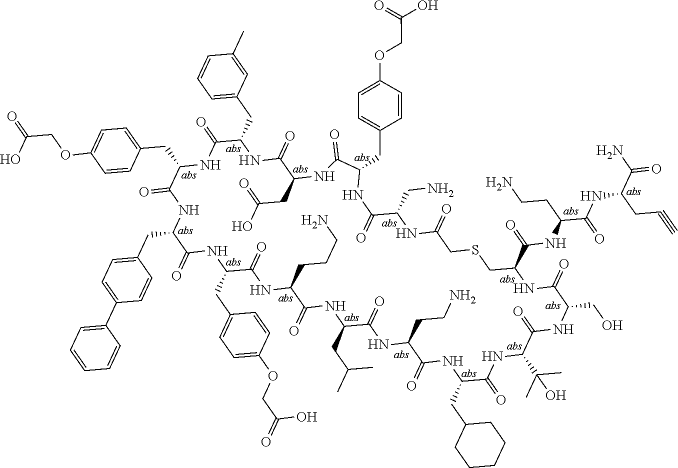 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1860 was prepared on a 25 μmol scale. The yield of the product was 10.1 mg, and its estimated purity by LCMS analysis was 84.7%. Analysis condition A: Retention time=1.59 min; ESI-MS(+) m/z [M+3H]³⁺: 763.1.

### Preparation of Compound 1861

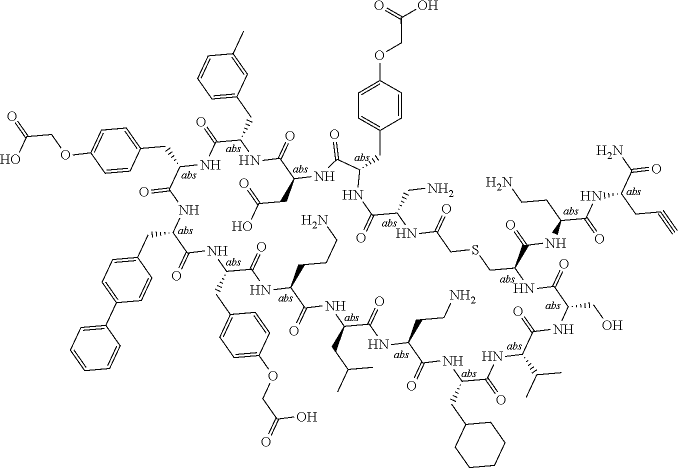 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1)C(N)=O]

Compound 1861 was prepared on a 25 μmol scale. The yield of the product was 1.4 mg, and its estimated purity by LCMS analysis was 95.9%. Analysis condition A: Retention time=1.49 min; ESI-MS(+) m/z [M+2H]²⁺: 1136.2.

### Preparation of Compound 1862

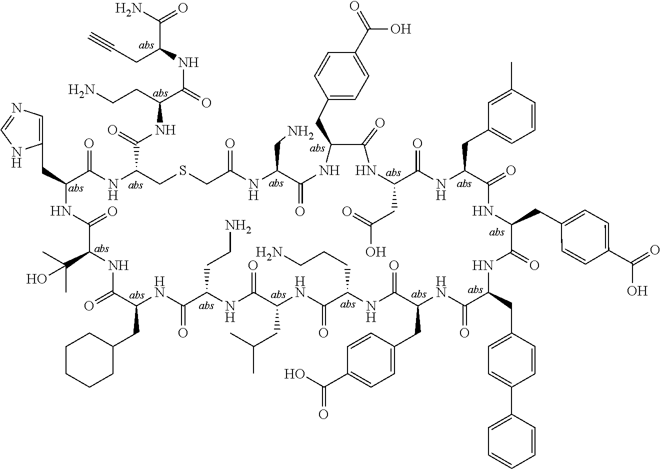 [SMILES: C#CC[C@H](NC(=O)[C@H](CCN)NC(=O)[C@@H]1CSCC(=O)N[C@@H](CN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](Cc2cnc[nH]2)C(=O)N1)C(N)=O]

Compound 1862 was prepared on a 25 μmol scale. The yield of the product was 3.6 mg, and its estimated purity by LCMS analysis was 85.2%. Analysis condition B: Retention time=1.5 min; ESI-MS(+) m/z [M+2H]²⁺: 1124.3.

### Preparation of Compound 1863

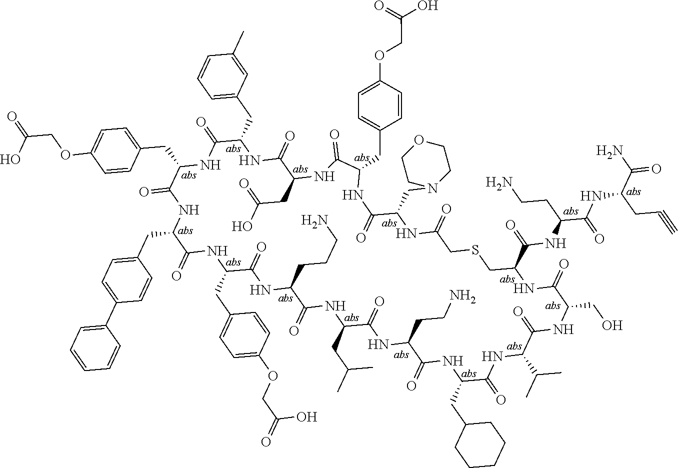 [SMILES: C#CC[C@H](NC(=O)[C@H](CCN)NC(=O)[C@@H]1CSCC(=O)N[C@@H](CN2CCOCC2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1)C(N)=O]

Compound 1863 was prepared on a 25 μmol scale. The yield of the product was 5.2 mg, and its estimated purity by LCMS analysis was 83.5%. Analysis condition A: Retention time=1.43 min; ESI-MS(+) m/z [M+2H]²⁺: 1171.2.

### Preparation of Compound 1864

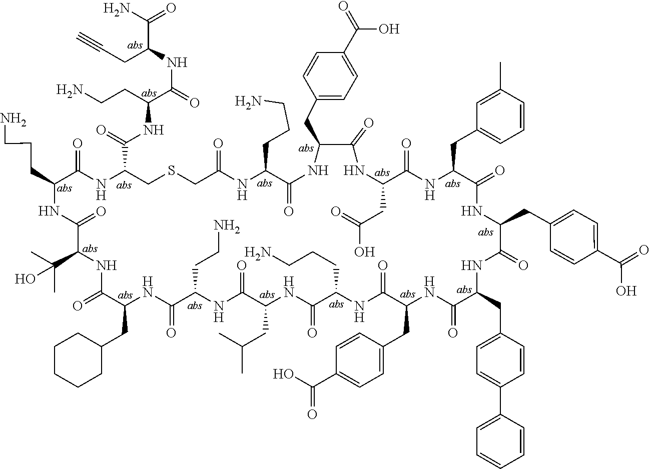 [SMILES: C#CC[C@H](NC(=O)[C@H](CCN)NC(=O)[C@@H]1CSCC(=O)N[C@@H](CCCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCN)C(=O)N1)C(N)=O]

Compound 1864 was prepared on a 50 μmol scale. The yield of the product was 12.5 mg, and its estimated purity by LCMS analysis was 100%. Analysis condition A: Retention time=1.38 min; ESI-MS(+) m/z [M+2H]²⁺: 1126.8.

### Preparation of Compound 1865

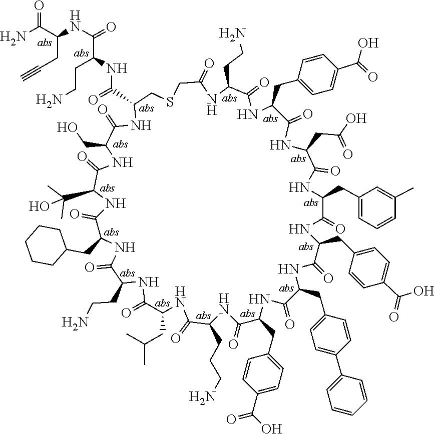 [SMILES: C#CC[C@H](NC(=O)[C@H](CCN)NC(=O)[C@@H]1CSCC(=O)N[C@@H](CCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 1865 was prepared on a 50 μmol scale. The yield of the product was 4.2 mg, and its estimated purity by LCMS analysis was 95.6%. Analysis condition B: Retention time=1.62 min; ESI-MS(+) m/z [M+2H]²⁺: 1106.2.

### Preparation of Compound 2000

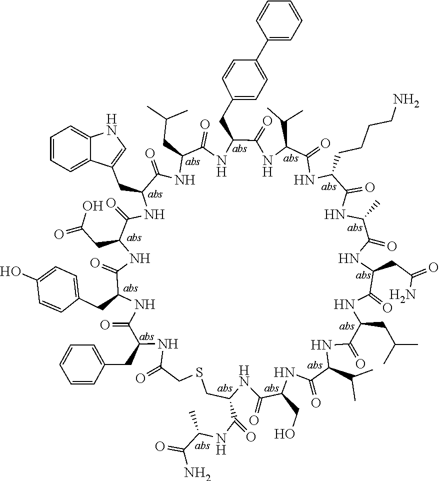

Compound 2000 was prepared on a 50 μmol scale. The yield of the product was 9 mg, and its estimated purity by LCMS analysis was 94.2%. Analysis condition B: Retention time=1.7 min; ESI-MS(+) m/z [M+H]⁺: 1891.

### Preparation of Compound 2001

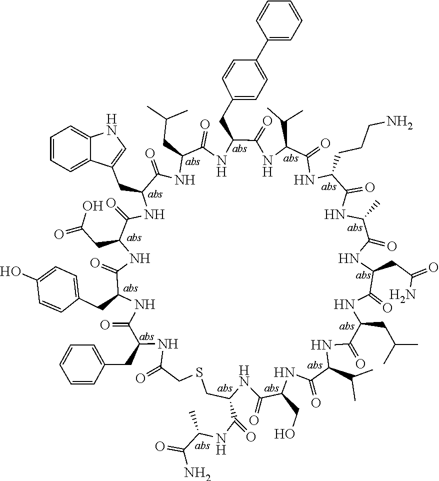

Compound 2001 was prepared on a 50 μmol scale. The yield of the product was 14.4 mg, and its estimated purity by LCMS analysis was 94.2%. Analysis condition A: Retention time=1.68 min; ESI-MS(+) m/z [M+H]⁺: 1877.

### Preparation of Compound 2002

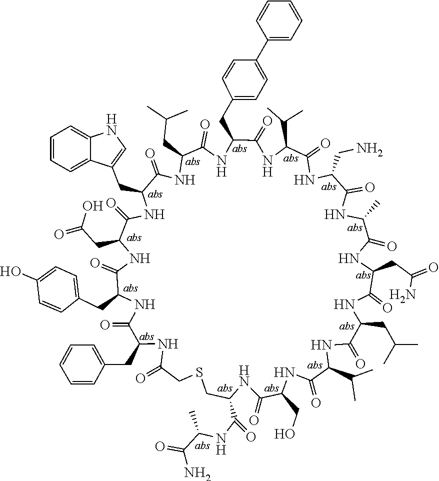

Compound 2002 was prepared on a 50 μmol scale. The yield of the product was 22.3 mg, and its estimated purity by LCMS analysis was 98.3%. Analysis condition A: Retention time=1.77 min; ESI-MS(+) m/z [M+H]⁺: 1849.9.

### Preparation of Compound 2003

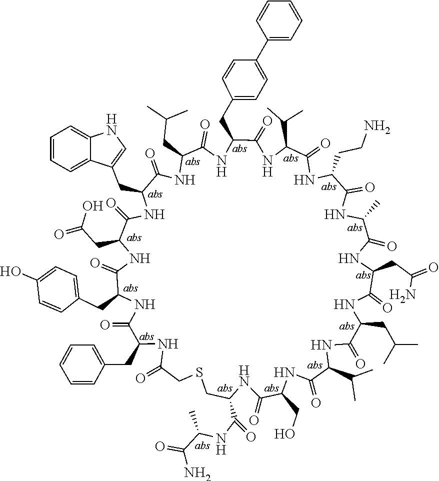

Compound 2003 was prepared on a 50 μmol scale. The yield of the product was 16 mg, and its estimated purity by LCMS analysis was 94.8%. Analysis condition A: Retention time=1.73 min; ESI-MS(+) m/z [M+H]⁺: 1864.

### Preparation of Compound 2004

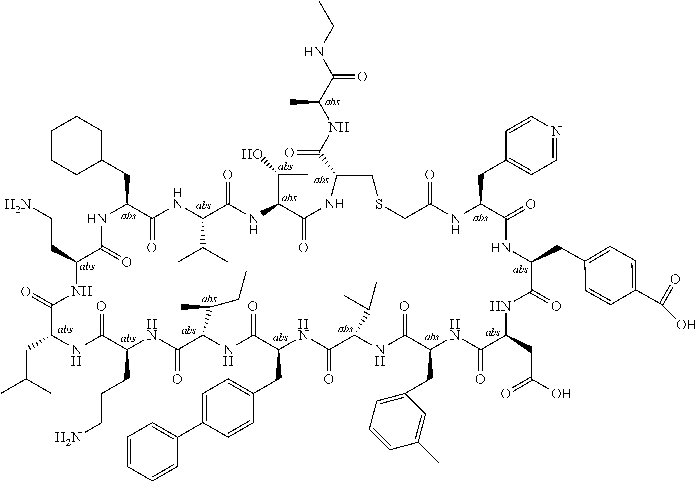 [SMILES: CCNC(=O)[C@H](C)NC(=O)[C@@H]1CSCC(=O)N[C@@H](Cc2ccncc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H]([C@@H](C)CC)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1]

Compound 2004 was prepared on a 50 μmol scale. The yield of the product was 1.8 mg, and its estimated purity by LCMS analysis was 100%. Analysis condition A: Retention time=1.8 min; ESI-MS(+) m/z [M+2H]²⁺: 996.2.

### Preparation of Compound 2005

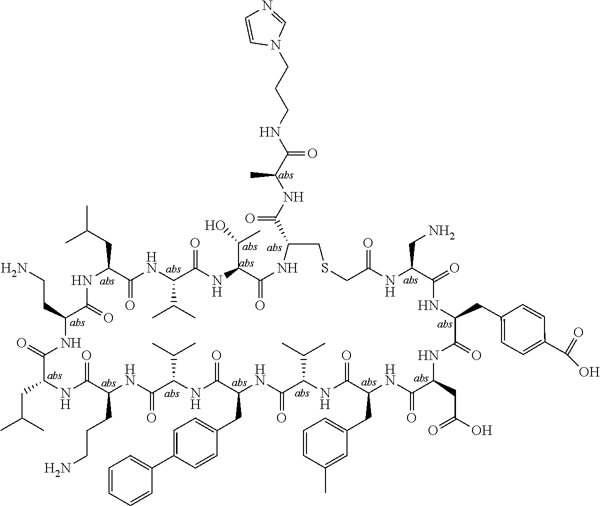 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](C)C(=O)NCCCn3ccnc3)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2005 was prepared on a 50 μmol scale. The yield of the product was 25.5 mg, and its estimated purity by LCMS analysis was 96.6%. Analysis condition B: Retention time=1.38 min; ESI-MS(+) m/z [M+H]⁺: 1954.3.

### Preparation of Compound 2006

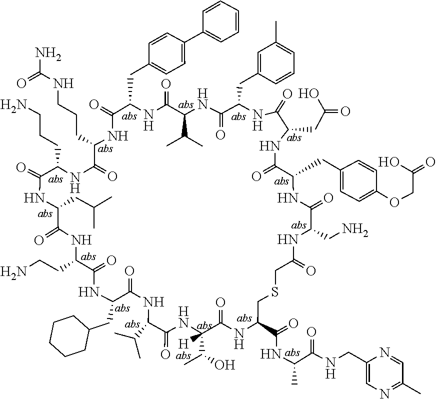 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(=O)NCc3cnc(C)cn3)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2006 was prepared on a 50 μmol scale. The yield of the product was 32.4 mg, and its estimated purity by LCMS analysis was 100%. Analysis condition A: Retention time=1.6, 1.65 min; ESI-MS(+) m/z [M+2H]²⁺: 1041.07, 1040.9.

### Preparation of Compound 2007

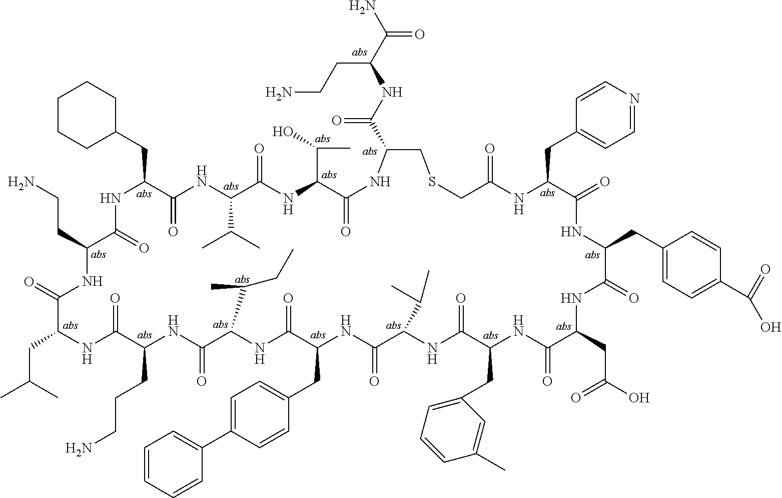 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2007 was prepared on a 50 μmol scale. The yield of the product was 4.1 mg, and its estimated purity by LCMS analysis was 81%. Analysis condition A: Retention time=1.63 min; ESI-MS(+) m/z [M+2H]²⁺: 997.2.

### Preparation of Compound 2008

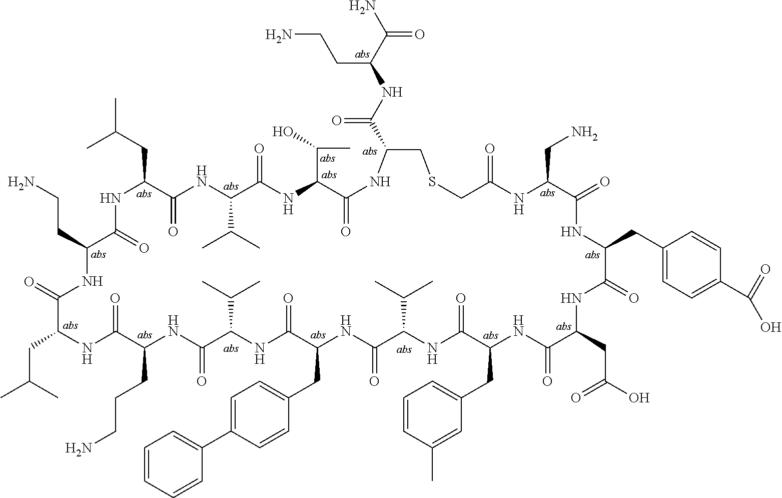 [SMILES: Cc1cccc(C[C@@H]2NC(=O)[C@H](CC(=O)O)NC(=O)[C@H](Cc3ccc(C(=O)O)cc3)NC(=O)[C@H](CN)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2008 was prepared on a 50 μmol scale. The yield of the product was 4.3 mg, and its estimated purity by LCMS analysis was 86.8%. Analysis condition B: Retention time=1.39 min; ESI-MS(+) m/z [M+2H]²⁺: 939.1.

### Preparation of Compound 2009

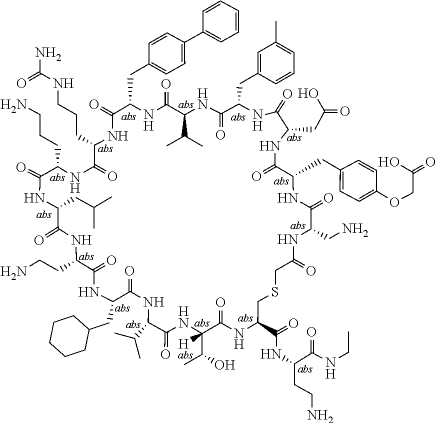 [SMILES: CCNC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1]

Compound 2009 was prepared on a 50 μmol scale. The yield of the product was 28.6 mg, and its estimated purity by LCMS analysis was 94.9%. Analysis condition A: Retention time=1.51 min; ESI-MS(+) m/z [M+2H]²⁺: 1017.2.

### Preparation of Compound 2010

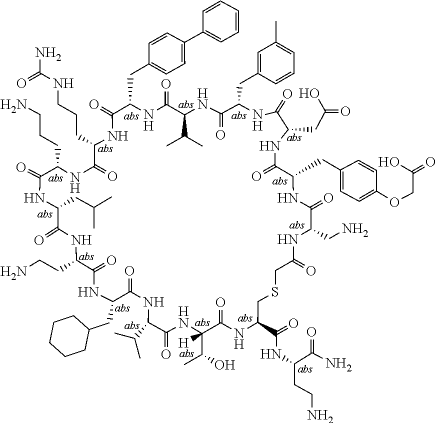 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2010 was prepared on a 50 μmol scale. The yield of the product was 2.3 mg, and its estimated purity by LCMS analysis was 100%. Analysis condition A: Retention time=1.49 min; ESI-MS(+) m/z [M+2H]²⁺: 1003.1.

### Preparation of Compound 2011

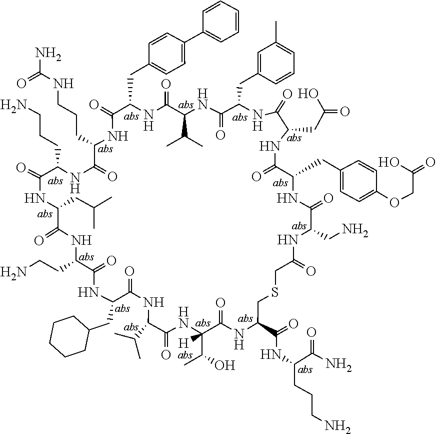 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCCN)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2011 was prepared on a 50 μmol scale. The yield of the product was 7 mg, and its estimated purity by LCMS analysis was 96.1%. Analysis condition A: Retention time=1.51 min; ESI-MS(+) m/z [M+2H]²⁺: 1010.2.

### Preparation of Compound 2012

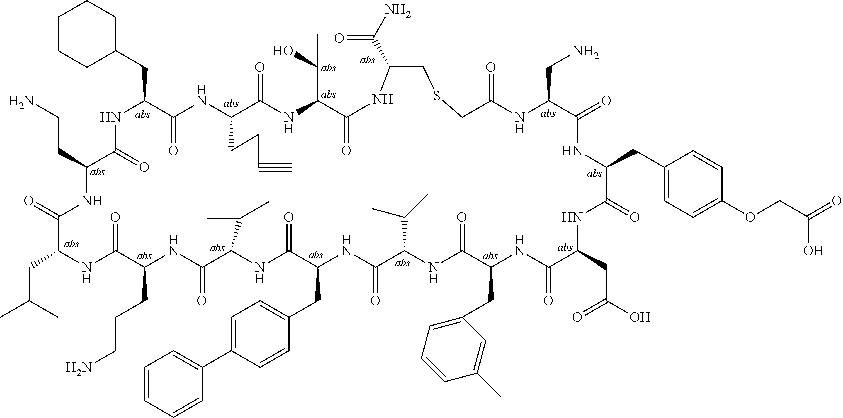

Compound 2012 was prepared on a 50 μmol scale. The yield of the product was 4.7 mg, and its estimated purity by LCMS analysis was 96.3%. Analysis condition A: Retention time=1.71 min; ESI-MS(+) m/z [M+H]⁺: 1856.

### Preparation of Compound 2013

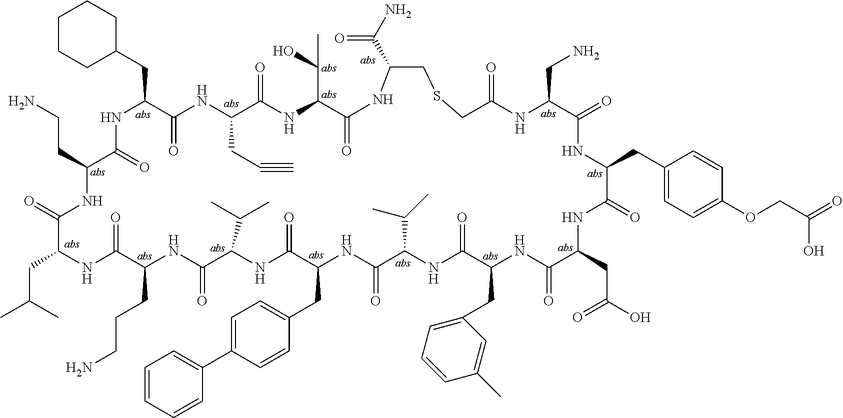

Compound 2013 was prepared on a 50 μmol scale. The yield of the product was 3.7 mg, and its estimated purity by LCMS analysis was 95.7%. Analysis condition B: Retention time=1.61 min; ESI-MS(+) m/z [M+H]⁺: 1842.1.

### Preparation of Compound 2014

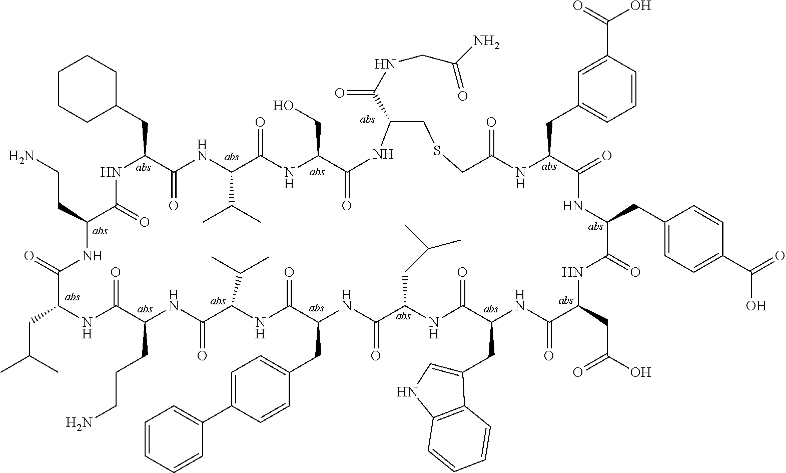

Compound 2014 was prepared on a 50 μmol scale. The yield of the product was 15.5 mg, and its estimated purity by LCMS analysis was 90.8%. Analysis condition B: Retention time=1.67 min; ESI-MS(+) m/z [M+2H]²⁺: 1002.2.

### Preparation of Compound 2015

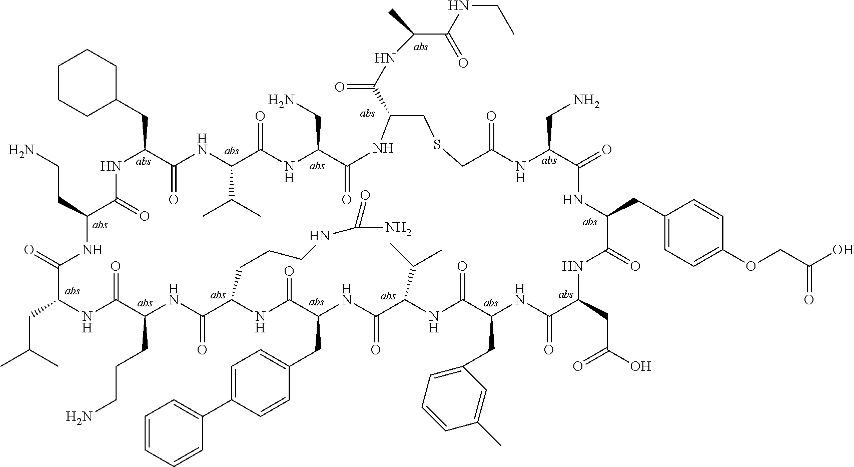

Compound 2015 was prepared on a 50 μmol scale. The yield of the product was 38.3 mg, and its estimated purity by LCMS analysis was 96.3%. Analysis condition B: Retention time=1.77 min; ESI-MS(+) m/z [M+H]⁺: 1988.

### Preparation of Compound 2016

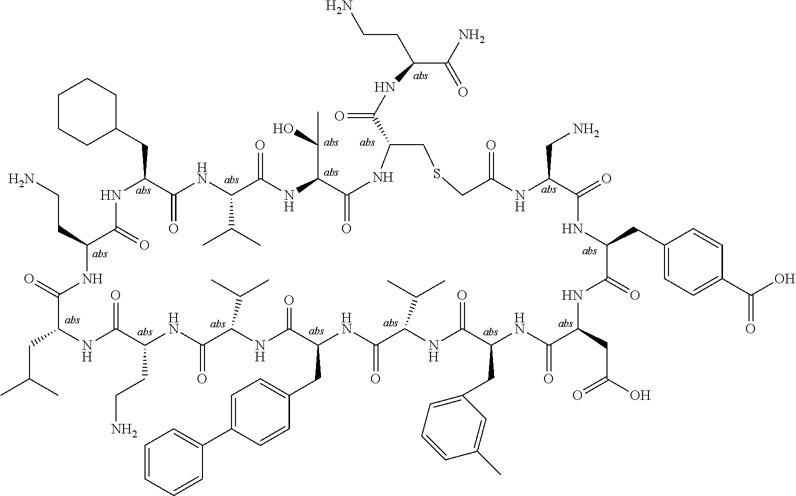

Compound 2016 was prepared on a 50 μmol scale. The yield of the product was 5.3 mg, and its estimated purity by LCMS analysis was 95.5%. Analysis condition A: Retention time=1.47 min; ESI-MS(+) m/z [M+2H]²⁺: 945.1.

### Preparation of Compound 2017

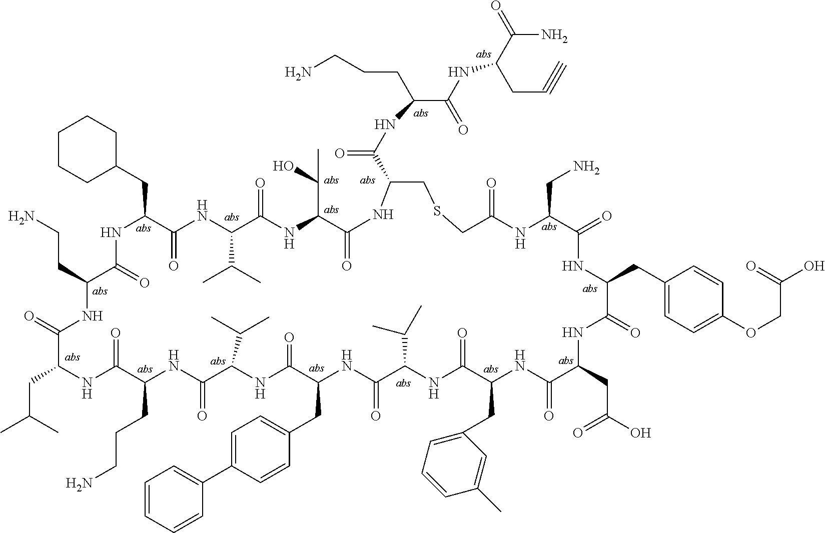

Compound 2017 was prepared on a 50 μmol scale. The yield of the product was 9.3 mg, and its estimated purity by LCMS analysis was 100%. Analysis condition A: Retention time=1.89 min; ESI-MS(+) m/z [M+3H]³⁺: 686.2.

### Preparation of Compound 2018

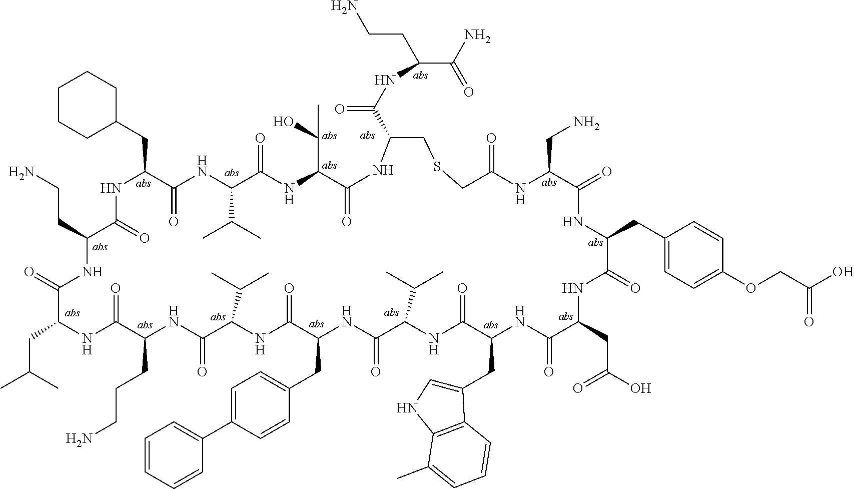

Compound 2018 was prepared on a 50 μmol scale. The yield of the product was 5.4 mg, and its estimated purity by LCMS analysis was 98.3%. Analysis condition B: Retention time=1.4 min; ESI-MS(+) m/z [M+3H]³⁺: 662.7.

### Preparation of Compound 2019

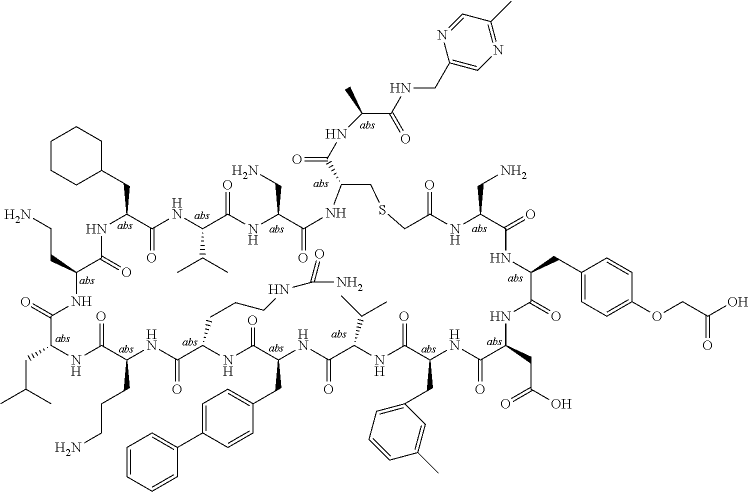

Compound 2019 was prepared on a 50 μmol scale. The yield of the product was 26.8 mg, and its estimated purity by LCMS analysis was 98.9%. Analysis condition A: Retention time=1.61 min; ESI-MS(+) m/z [M+2H]²⁺: 1034.

### Preparation of Compound 2020

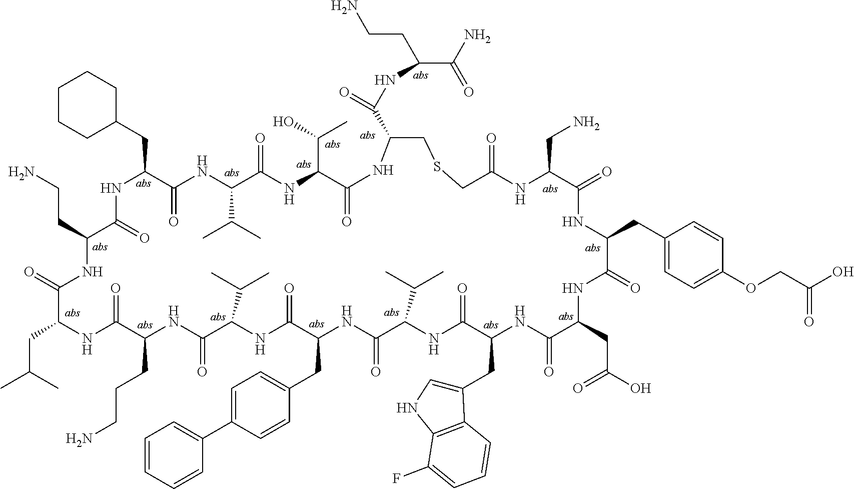

Compound 2020 was prepared on a 50 μmol scale. The yield of the product was 3.5 mg, and its estimated purity by LCMS analysis was 96.5%. Analysis condition B: Retention time=1.39 min; ESI-MS(+) m/z [M+2H]²⁺: 995.9.

### Preparation of Compound 2021

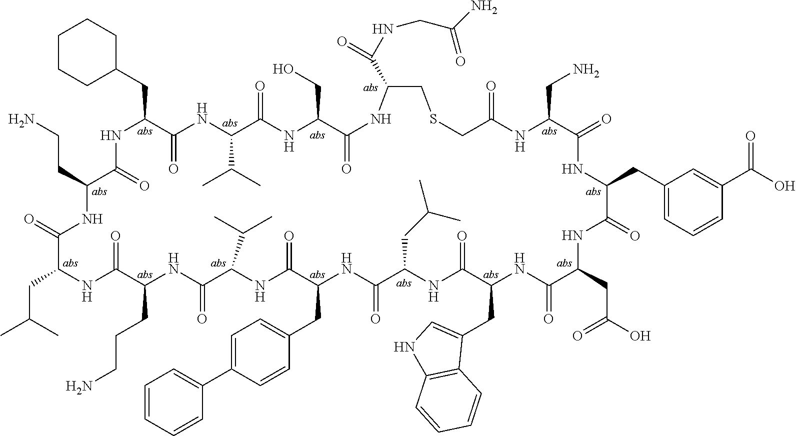 [SMILES: CC(C)C[C@@H]1NC(=O)[C@H](Cc2c[nH]c3ccccc23)NC(=O)[C@H](CC(=O)O)NC(=O)[C@H](Cc2cccc(C(=O)O)c2)NC(=O)[C@H](CN)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 2021 was prepared on a 50 μmol scale. The yield of the product was 4.6 mg, and its estimated purity by LCMS analysis was 95.4%. Analysis condition A: Retention time=1.6 min; ESI-MS(+) m/z [M+2H]²⁺: 950.

### Preparation of Compound 2022

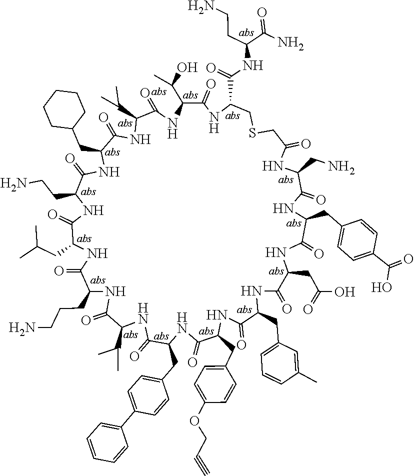

Compound 2022 was prepared on a 50 μmol scale. The yield of the product was 10.5 mg, and its estimated purity by LCMS analysis was 100%. Analysis condition A: Retention time=1.67 min; ESI-MS(+) m/z [M+2H]²⁺: 1010.1.

### Preparation of Compound 2023

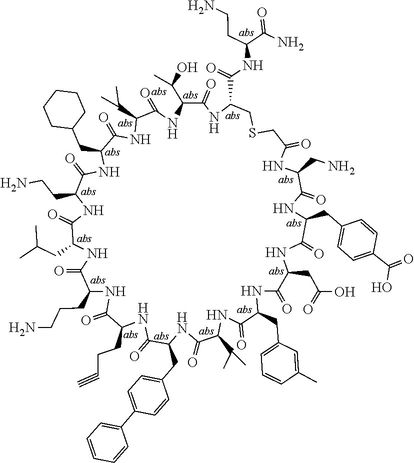

Compound 2023 was prepared on a 50 μmol scale. The yield of the product was 6.4 mg, and its estimated purity by LCMS analysis was 82.7%. Analysis condition A: Retention time=1.51 min; ESI-MS(+) m/z [M+H]⁺: 1926.1.

### Preparation of Compound 2024

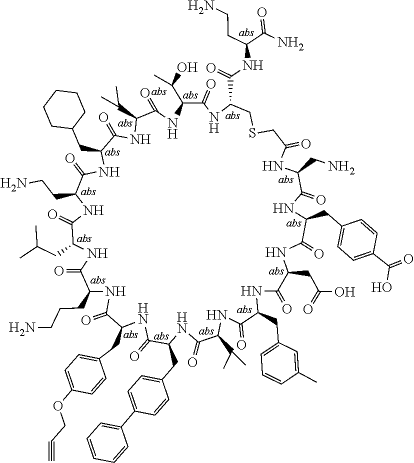

Compound 2024 was prepared on a 50 μmol scale. The yield of the product was 12.1 mg, and its estimated purity by LCMS analysis was 100%. Analysis condition A: Retention time=1.6 min; ESI-MS(+) m/z [M+2H]²⁺: 1010.2.

### Preparation of Compound 2025

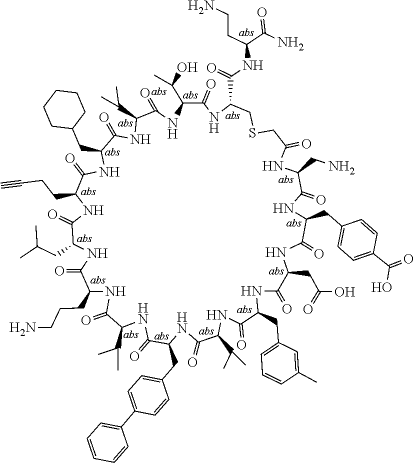

Compound 2025 was prepared on a 50 μmol scale. The yield of the product was 3.6 mg, and its estimated purity by LCMS analysis was 90.6%. Analysis condition A: Retention time=1.74 min; ESI-MS(+) m/z [M+H]⁺: 1925.

### Preparation of Compound 2026

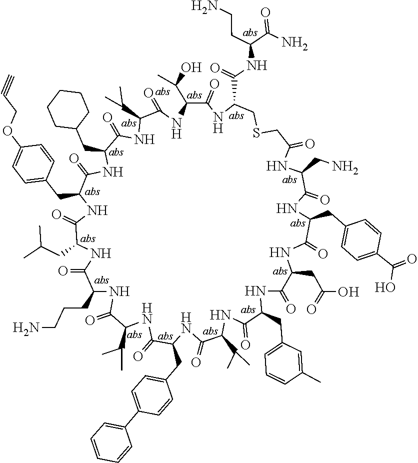

Compound 2026 was prepared on a 50 μmol scale. The yield of the product was 5.6 mg, and its estimated purity by LCMS analysis was 95.3%. Analysis condition B: Retention time=1.75 min; ESI-MS(+) m/z [M+2H]²⁺: 1009.3.

### Preparation of Compound 2027

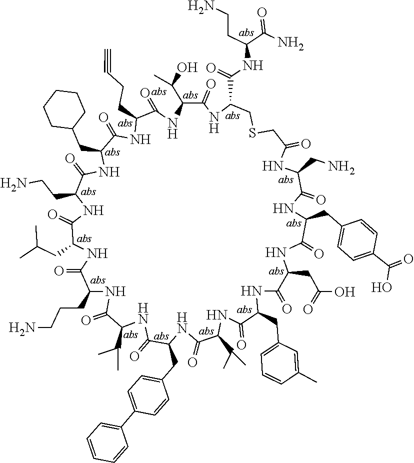

Compound 2027 was prepared on a 50 μmol scale. The yield of the product was 4.8 mg, and its estimated purity by LCMS analysis was 85.7%. Analysis condition B: Retention time=1.39 min; ESI-MS(+) m/z [M+3H]³⁺: 643.2.

### Preparation of Compound 2028

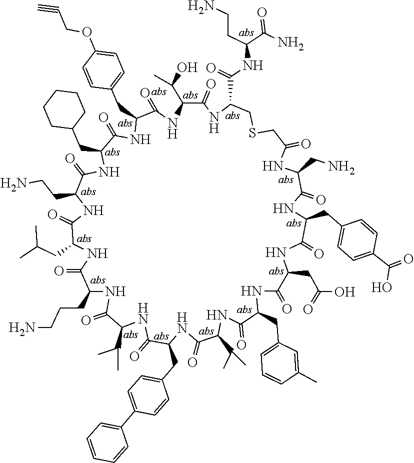

Compound 2028 was prepared on a 50 μmol scale. The yield of the product was 11.4 mg, and its estimated purity by LCMS analysis was 96%. Analysis condition B: Retention time=1.57 min; ESI-MS(+) m/z [M+2H]²⁺: 1010.1.

### Preparation of Compound 2029

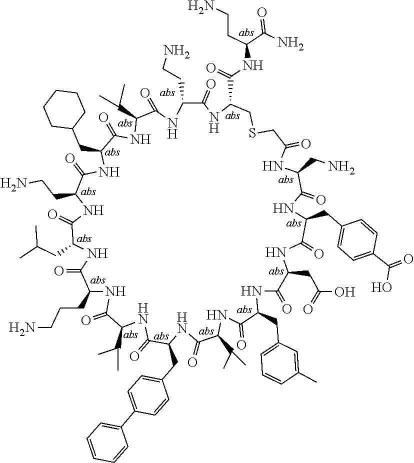

Compound 2029 was prepared on a 50 μmol scale. The yield of the product was 12.2 mg, and its estimated purity by LCMS analysis was 94%. Analysis condition B: Retention time=1.45 min; ESI-MS(+) m/z [M+H]⁺: 1916.1.

### Preparation of Compound 2030

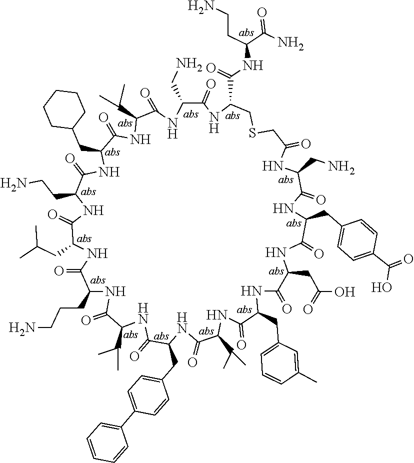

Compound 2030 was prepared on a 50 μmol scale. The yield of the product was 4.7 mg, and its estimated purity by LCMS analysis was 98.5%. Analysis condition A: Retention time=1.39 min; ESI-MS(+) m/z [M+H]⁺: 1901.3.

### Preparation of Compound 2031

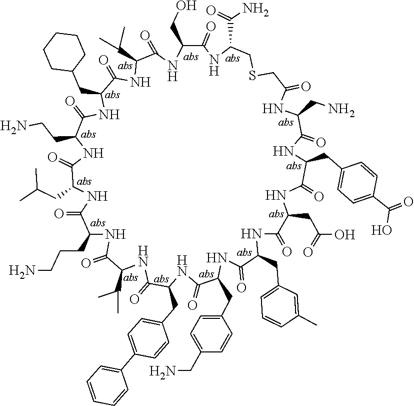

Compound 2031 was prepared on a 50 μmol scale. The yield of the product was 17.9 mg, and its estimated purity by LCMS analysis was 95.9%. Analysis condition A: Retention time=1.55 min; ESI-MS(+) m/z [M+2H]²⁺: 940.2.

### Preparation of Compound 2032

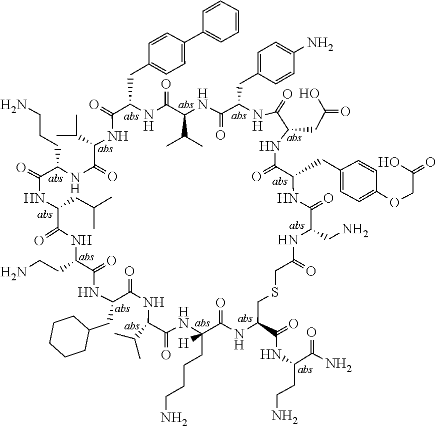 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(N)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(N)=O)NC(=O)[C@H](CCCCN)NC(=O)[C@H](C(C)C)NC(=O)[C@H](CC2CCCCC2)NC(=O)[C@H](CCN)NC1=O]

Compound 2032 was prepared on a 50 μmol scale. The yield of the product was 2.6 mg, and its estimated purity by LCMS analysis was 90%. Analysis condition A: Retention time=1.3 min; ESI-MS(+) m/z [M+H]⁺: 1973.9.

### Preparation of Compound 2033

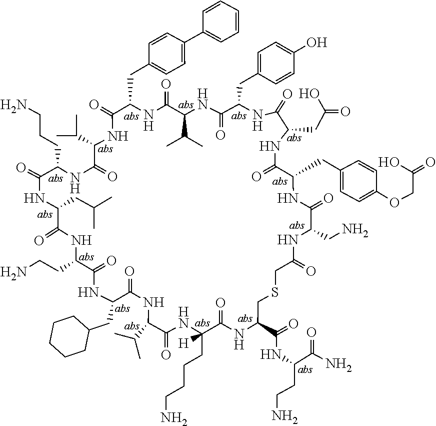 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(O)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(N)=O)NC(=O)[C@H](CCCCN)NC(=O)[C@H](C(C)C)NC(=O)[C@H](CC2CCCCC2)NC(=O)[C@H](CCN)NC1=O]

Compound 2033 was prepared on a 50 μmol scale. The yield of the product was 3.5 mg, and its estimated purity by LCMS analysis was 94.4%. Analysis condition B: Retention time=1.25 min; ESI-MS(+) m/z [M+2H]²⁺: 988.8.

### Preparation of Compound 2034

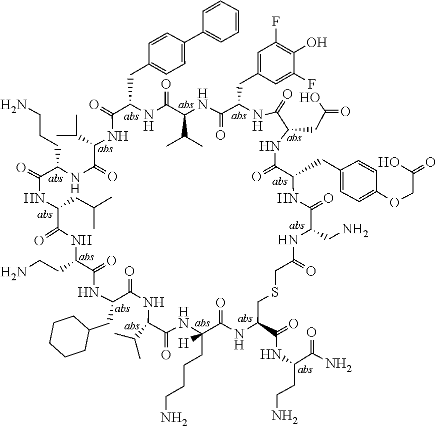 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2cc(F)c(O)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(N)=O)NC(=O)[C@H](CCCCN)NC(=O)[C@H](C(C)C)NC(=O)[C@H](CC2CCCCC2)NC(=O)[C@H](CCN)NC1=O]

Compound 2034 was prepared on a 50 μmol scale. The yield of the product was 2.7 mg, and its estimated purity by LCMS analysis was 97.8%. Analysis condition A: Retention time=1.33, 1.39, 1.43, 1.47, 1.55 min; ESI-MS(+) m/z [M+3H]³⁺: 671.24, 671.22, 671.2, 671.34, 1006.67.

### Preparation of Compound 2035

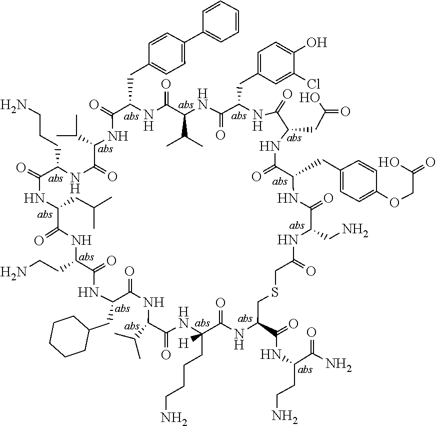 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2ccc(O)c(Cl)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CCN)C(N)=O)NC(=O)[C@H](CCCCN)NC(=O)[C@H](C(C)C)NC(=O)[C@H](CC2CCCCC2)NC(=O)[C@H](CCN)NC1=O]

Compound 2035 was prepared on a 50 μmol scale. The yield of the product was 1.2 mg, and its estimated purity by LCMS analysis was 96%. Analysis condition B: Retention time=1.25 min; ESI-MS(+) m/z [M+2H]²⁺: 1006.2.

### Preparation of Compound 2036

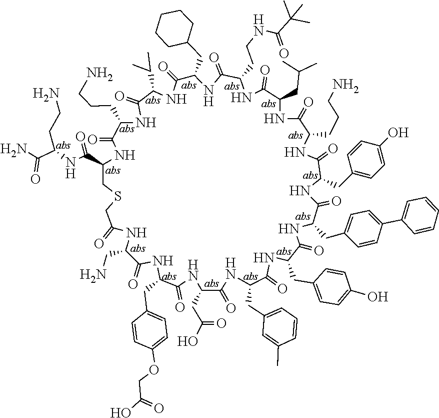 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCNC(=O)C(C)(C)C)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2036 was prepared on a 50 μmol scale. The yield of the product was 11.5 mg, and its estimated purity by LCMS analysis was 89.5%. Analysis condition B: Retention time=1.62, 1.65 min; ESI-MS(+) m/z [M+2H]²⁺: 1086.23, 1086.19.

### Preparation of Compound 2037

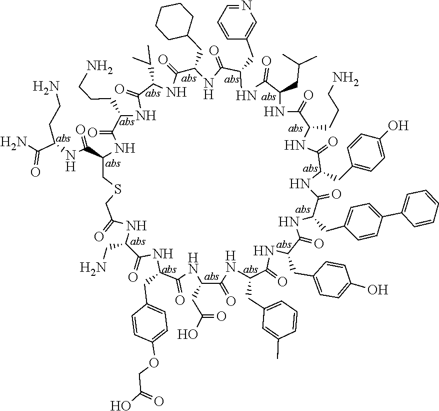 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](Cc3cccnc3)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2037 was prepared on a 50 μmol scale. The yield of the product was 7.9 mg, and its estimated purity by LCMS analysis was 90.3%. Analysis condition A: Retention time=1.66 min; ESI-MS(+) m/z [M+2H]²⁺: 1068.2.

### Preparation of Compound 2038

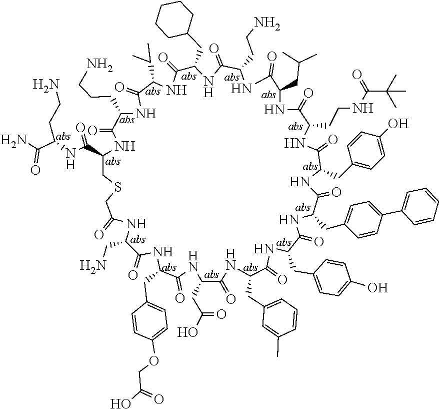 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCNC(=O)C(C)(C)C)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2038 was prepared on a 50 μmol scale. The yield of the product was 21.5 mg, and its estimated purity by LCMS analysis was 100%. Analysis condition A: Retention time=1.81 min; ESI-MS(+) m/z [M+3H]³⁺: 720.3.

### Preparation of Compound 2039

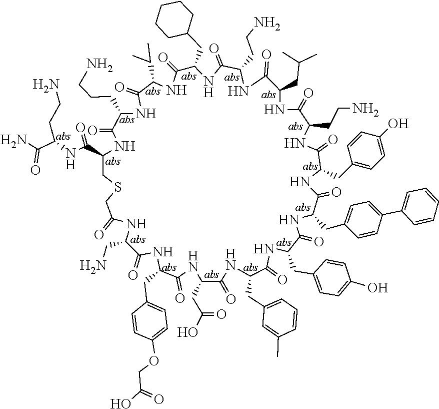 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@@H](CCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2039 was prepared on a 50 μmol scale. The yield of the product was 25.2 mg, and its estimated purity by LCMS analysis was 100%. Analysis condition A: Retention time=1.52 min; ESI-MS(+) m/z [M+2H]²⁺: 1037.2.

### Preparation of Compound 2040

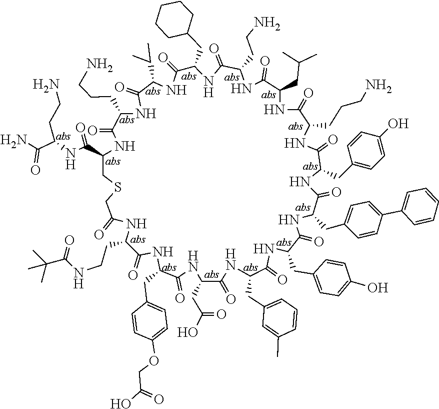 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)C(C)(C)C)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2040 was prepared on a 50 μmol scale. The yield of the product was 14.6 mg, and its estimated purity by LCMS analysis was 95.7%. Analysis condition B: Retention time=1.53 min; ESI-MS(+) m/z [M+3H]³⁺: 729.

### Preparation of Compound 2041

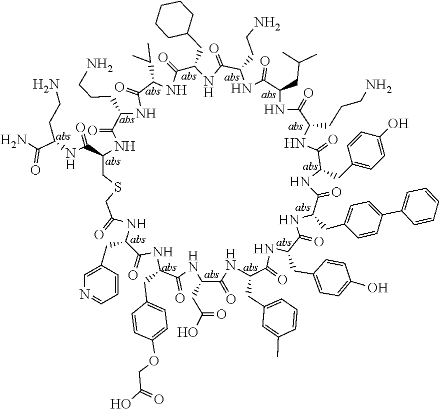 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccnc3)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2041 was prepared on a 50 μmol scale. The yield of the product was 31.3 mg, and its estimated purity by LCMS analysis was 100%. Analysis condition A: Retention time=1.49 min; ESI-MS(+) m/z [M+2H]²⁺: 1075.2.

### Preparation of Compound 2042

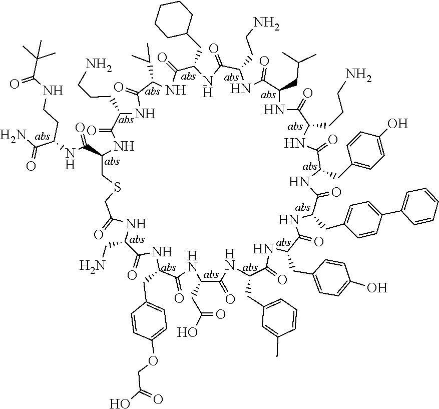 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCNC(=O)C(C)(C)C)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2042 was prepared on a 50 μmol scale. The yield of the product was 53.8 mg, and its estimated purity by LCMS analysis was 97.5%. Analysis condition B: Retention time=1.51 min; ESI-MS(+) m/z [M+3H]³⁺: 725.

### Preparation of Compound 2043

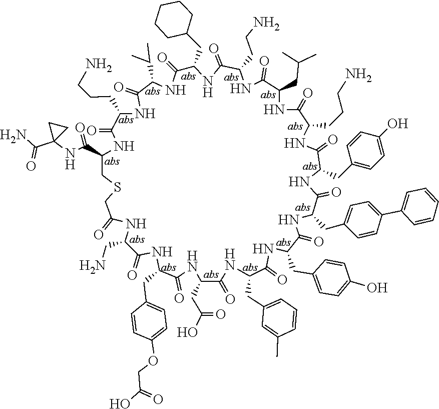 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NC3(C(N)=O)CC3)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2043 was prepared on a 50 μmol scale. The yield of the product was 67.7 mg, and its estimated purity by LCMS analysis was 97.9%. Analysis condition A: Retention time=1.54 min; ESI-MS(+) m/z [M+2H]²⁺: 1036.1.

### Preparation of Compound 2044

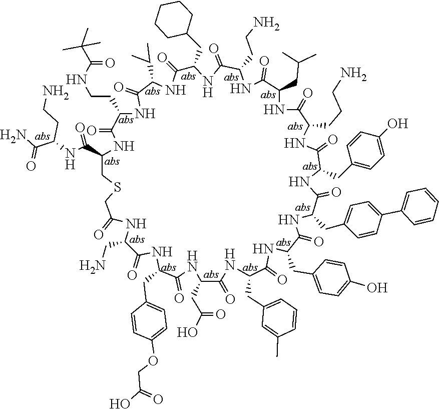 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCNC(=O)C(C)(C)C)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2044 was prepared on a 50 μmol scale. The yield of the product was 11.2 mg, and its estimated purity by LCMS analysis was 98.8%. Analysis condition A: Retention time=2.05 min; ESI-MS(+) m/z [M+3H]³⁺: 720.

### Preparation of Compound 2045

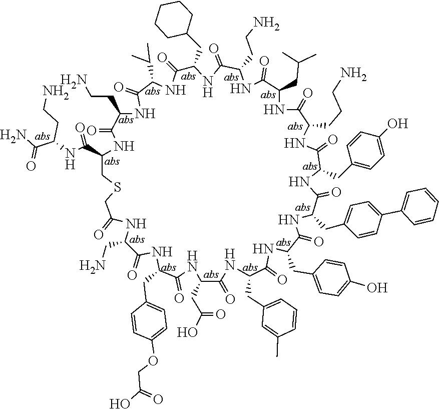 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@@H](CCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2045 was prepared on a 50 μmol scale. The yield of the product was 15.8 mg, and its estimated purity by LCMS analysis was 96.5%. Analysis condition A: Retention time=1.33 min; ESI-MS(+) m/z [M+3H]³⁺: 692.1.

### Preparation of Compound 2046

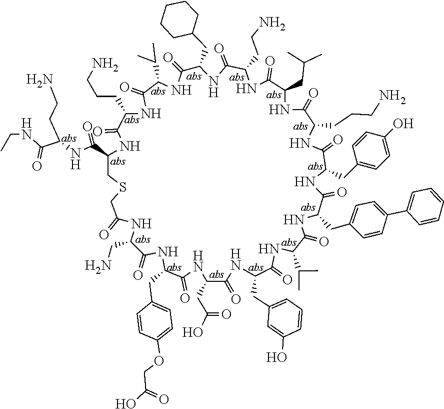 [SMILES: CCNC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(O)c2)C(=O)N[C@@H](C(C)C)C(=O)N[C@@H](Cc2ccc(-c3ccccc3)cc2)C(=O)N[C@@H](Cc2ccc(O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCCN)C(=O)N1]

Compound 2046 was prepared on a 50 μmol scale. The yield of the product was 30.8 mg, and its estimated purity by LCMS analysis was 84.3%. Analysis condition B: Retention time=1.37 min; ESI-MS(+) m/z [M+3H]³⁺: 685.3.

### Preparation of Compound 2047

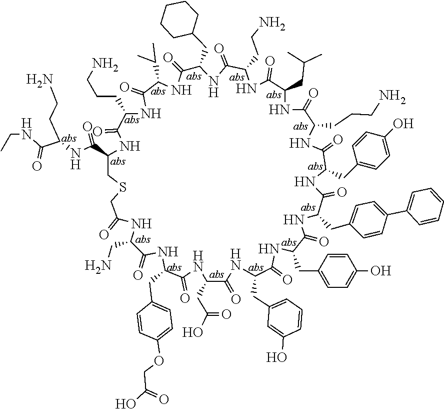 [SMILES: CCNC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(O)c2)C(=O)N[C@@H](Cc2ccc(O)cc2)C(=O)N[C@@H](Cc2ccc(-c3ccccc3)cc2)C(=O)N[C@@H](Cc2ccc(O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCCN)C(=O)N1]

Compound 2047 was prepared on a 50 μmol scale. The yield of the product was 67.6 mg, and its estimated purity by LCMS analysis was 88.2%. Analysis condition A: Retention time=1.4 min; ESI-MS(+) m/z [M+3H]³⁺: 706.9.

### Preparation of Compound 2048

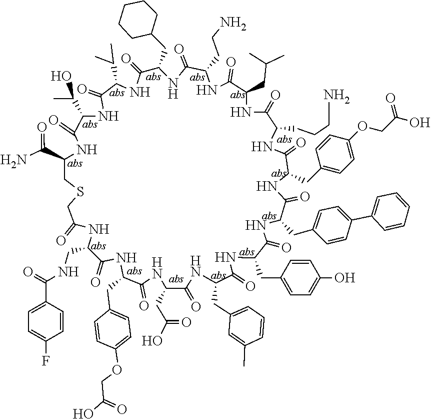 [SMILES: Cc1cccc(C[C@@H]2NC(=O)[C@H](CC(=O)O)NC(=O)[C@H](Cc3ccc(OCC(=O)O)cc3)NC(=O)[C@H](CNC(=O)c3ccc(F)cc3)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

The linear peptide containing Dap (Mtt) (total 100 μmol) on Rink resin was transferred into a Bio-Rad tube with a frit. The resin was washed 3 times with CH₂Cl₂. About 5 mL of 1.5% TFA in CH₂Cl₂ was added and the vessel was shaken for 3-5 min. The solvents were drained. The deprotection was repeated two more times. The resin containing the Mtt-free Dap residue was then resined with CH2Cl2 (5×). The resin was divided into 4 vessels with the frit. To each vessel, DMF (5 mL) was added and the vessel was shaken for 10 min. DMF was drained. 3-5 mL of fresh DMF, DIEA (0.1 mL) was added followed by 50 mg of 2,5-dioxopyrrolidin-1-yl 4-fluorobenzoate (or other activated esters or acyl chlorides in other reactions). The mixture was shaken for 2 h at rt. It was drained, rinsed with DMF (5 x), then CH₂Cl₂ (3 x), and dried. About 4-5 mL of TFA/TIS/DTT (96:3:1) was added and the vessel was shaken for 1.5 h at rt. The TFA solution was drained through the frit and into a vial. Et₂O (40 mL) was added. The cold vessel was centrifuged (2 x) and the solids were collected and air dried. The solids were dissolved in DMF and 1.5-2 mL of DIEA was added. The resulting solution was shaken overnight. It was concentrated and the residue was dissolved in 1.5-2 mL of DMF and submitted to purification. Compound 2048 was prepared on a 25 μmol scale. The yield of the product was 16.7 mg, and its estimated purity by LCMS analysis was 95.4%. Analysis condition B: Retention time=1.75 min; ESI-MS(+) m/z [M+2H]²⁺: 1077.8.

### Preparation of Compound 2049

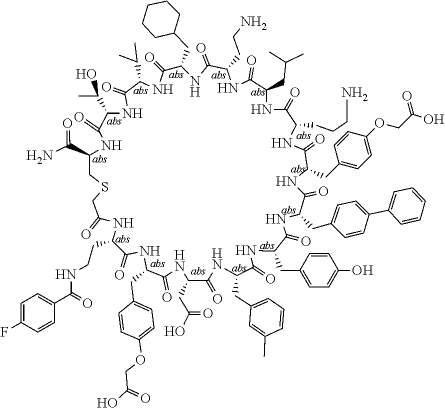 [SMILES: Cc1cccc(C[C@@H]2NC(=O)[C@H](CC(=O)O)NC(=O)[C@H](Cc3ccc(OCC(=O)O)cc3)NC(=O)[C@H](CCNC(=O)c3ccc(F)cc3)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2049 was prepared on a 25 μmol scale. The yield of the product was 1.6 mg, and its estimated purity by LCMS analysis was 97.5%. Analysis condition B: Retention time=1.76 min; ESI-MS(+) m/z [M+2H]²⁺: 1084.9.

### Preparation of Compound 2050

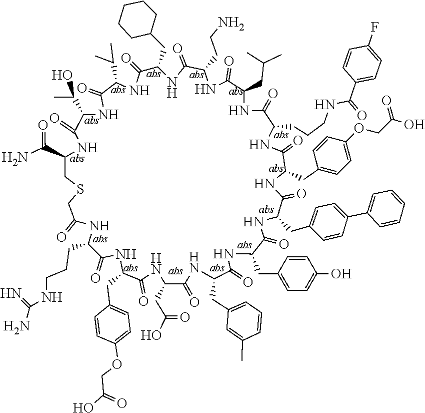 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCNC(=O)c3ccc(F)cc3)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2050 was prepared on a 25 μmol scale. The yield of the product was 1.9 mg, and its estimated purity by LCMS analysis was 92.7%. Analysis condition A: Retention time=1.64 min; ESI-MS(+) m/z [M+2H]²⁺: 1112.9.

### Preparation of Compound 2051

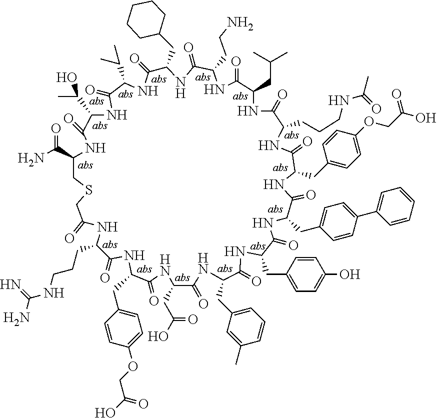 [SMILES: CC(=O)NCCC[C@@H]1NC(=O)[C@H](Cc2ccc(OCC(=O)O)cc2)NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC1=O]

Compound 2051 was prepared on a 25 μmol scale. The yield of the product was 4.2 mg, and its estimated purity by LCMS analysis was 96.5%. Analysis condition A: Retention time=1.5 min; ESI-MS(+) m/z [M+2H]²⁺: 1073.1.

### Preparation of Compound 2052

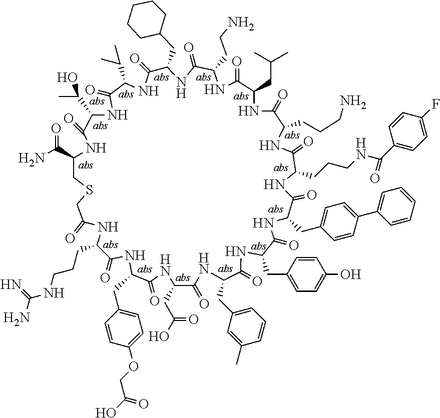 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(=O)c3ccc(F)cc3)NC(=O)[C@H](Cc3ccc(-c4ccccc4)cc3)NC(=O)[C@H](Cc3ccc(O)cc3)NC2=O)c1]

Compound 2052 was prepared on a 25 μmol scale. The yield of the product was 3.1 mg, and its estimated purity by LCMS analysis was 100%. Analysis condition A: Retention time=1.68 min; ESI-MS(+) m/z [M+2H]²⁺: 1060.

### Preparation of Compound 2053

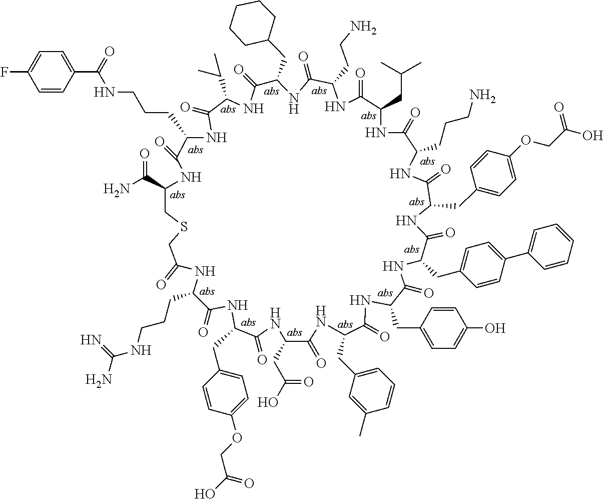 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H](CCCNC(=O)c3ccc(F)cc3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2053 was prepared on a 25 μmol scale. The yield of the product was mg, and its estimated purity by LCMS analysis was 96.7%. Analysis condition B: Retention time=1.68 min; ESI-MS(+) m/z [M+2H]²⁺: 1118.9.

### Preparation of Compound 2054

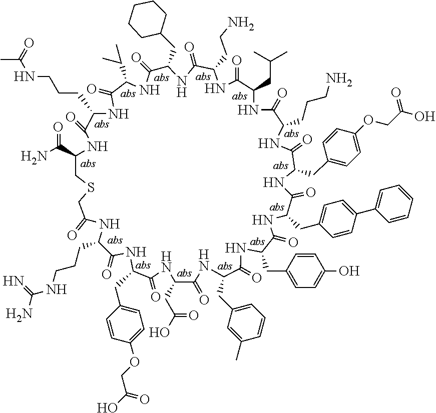 [SMILES: CC(=O)NCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(O)cc2)NC(=O)[C@H](Cc2cccc(C)c2)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(N)=O)NC1=O]

Compound 2054 was prepared on a 25 μmol scale. The yield of the product was mg, and its estimated purity by LCMS analysis was 100%. Analysis condition A: Retention time=1.47 min; ESI-MS(+) m/z [M+2H]²⁺: 1079.3.

### Preparation of Compound 2055

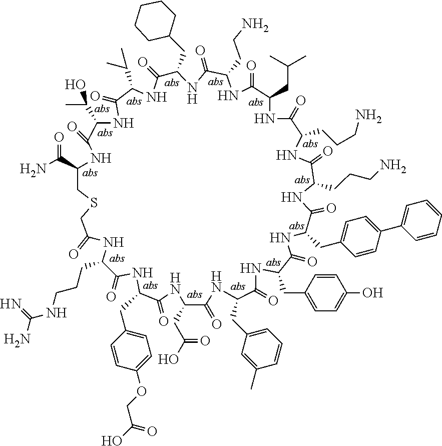 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](Cc3ccc(O)cc3)NC2=O)c1]

Compound 2055 was prepared on a 25 μmol scale. The yield of the product was 8.8 mg, and its estimated purity by LCMS analysis was 92.8%. Analysis condition B: Retention time=1.52 min; ESI-MS(+) m/z [M+2H]²⁺: 998.1.

### Preparation of Compound 2056

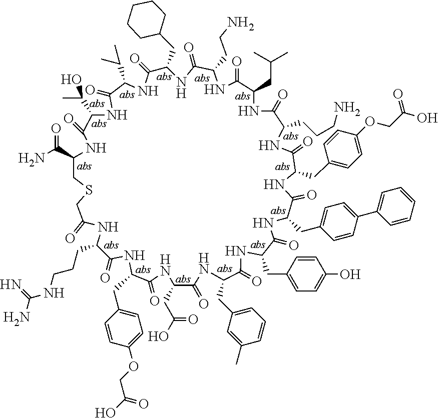 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(O)cc3)NC2=O)c1]

Compound 2056 was prepared on a 25 μmol scale. The yield of the product was 7.1 mg, and its estimated purity by LCMS analysis was 91.5%. Analysis condition A: Retention time=1.5 min; ESI-MS(+) m/z [M+2H]²⁺: 1052.2.

### Preparation of Compound 2057

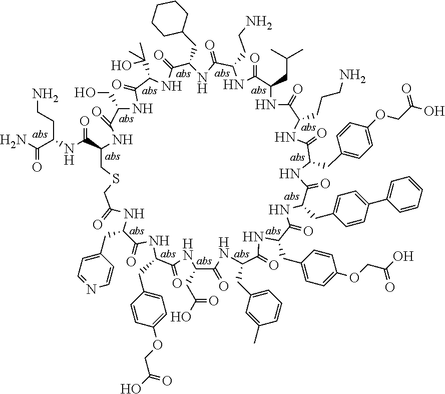 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2057 was prepared on a 50 μmol scale. The yield of the product was 5.7 mg, and its estimated purity by LCMS analysis was 95.3%. Analysis condition A: Retention time=1.42 min; ESI-MS(+) m/z [M+2H]²⁺: 1128.

### Preparation of Compound 2058

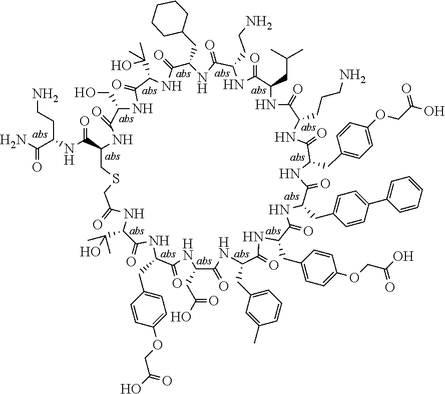 [SMILES: Cc1cccc(C[C@@H]2NC(=O)[C@H](CC(=O)O)NC(=O)[C@H](Cc3ccc(OCC(=O)O)cc3)NC(=O)[C@H](C(C)(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2058 was prepared on a 50 μmol scale. The yield of the product was 5.2 mg, and its estimated purity by LCMS analysis was 100%. Analysis condition A: Retention time=1.36 min; ESI-MS(+) m/z [M+2H]²⁺: 1111.0.

### Preparation of Compound 2059

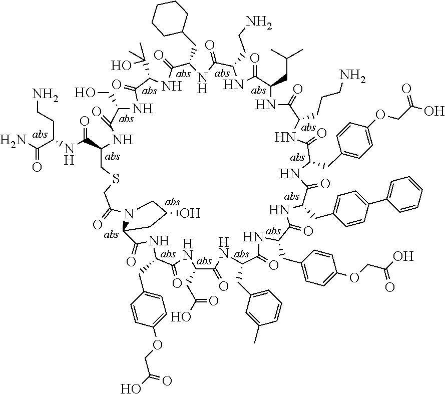 [SMILES: Cc1cccc(C[C@@H]2NC(=O)[C@H](CC(=O)O)NC(=O)[C@H](Cc3ccc(OCC(=O)O)cc3)NC(=O)[C@@H]3C[C@@H](O)CN3C(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2059 was prepared on a 50 μmol scale. The yield of the product was 5.6 mg, and its estimated purity by LCMS analysis was 95.2%. Analysis condition A: Retention time=1.38 min; ESI-MS(+) m/z [M+2H]²⁺: 1110.2.

### Preparation of Compound 2060

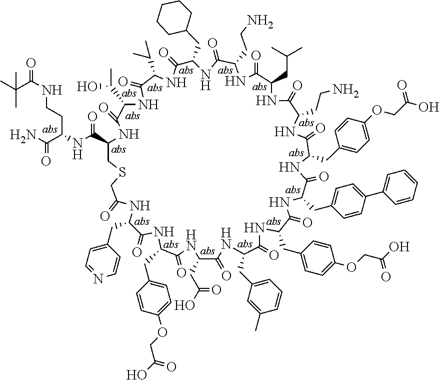 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCNC(=O)C(C)(C)C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2060 was prepared on a 25 μmol scale. The yield of the product was 7 mg, and its estimated purity by LCMS analysis was 95.1%. Analysis condition: Retention time=1.6 min; ESI-MS(+) m/z [M+2H]²⁺: 1162.1.

### Preparation of Compound 2061

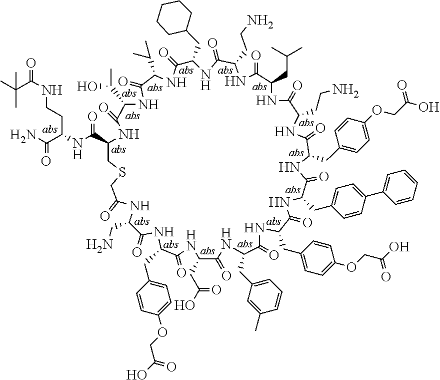 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCNC(=O)C(C)(C)C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2061 was prepared on a 25 μmol scale. The yield of the product was 14.4 mg, and its estimated purity by LCMS analysis was 90.8%. Analysis condition A: Retention time=1.63 min; ESI-MS(+) m/z [M+2H]²⁺: 1131.

### Preparation of Compound 2062

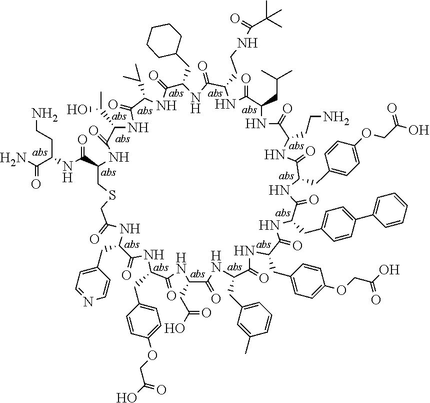 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCNC(=O)C(C)(C)C)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2062 was prepared on a 25 μmol scale. The yield of the product was 3.1 mg, and its estimated purity by LCMS analysis was 95.4%. Analysis condition B: Retention time=1.79 min; ESI-MS(+) m/z [M+2H]²⁺: 1162.2.

### Preparation of Compound 2063

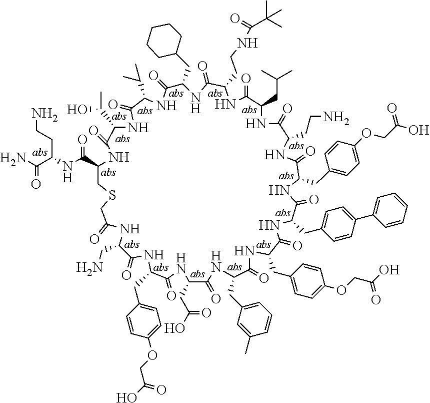 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCNC(=O)C(C)(C)C)NC(=O)[C@@H](CC(C)C)NC(=O)[C@H](CCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2063 was prepared on a 25 μmol scale. The yield of the product was 4.7 mg, and its estimated purity by LCMS analysis was 96%. Analysis condition A: Retention time=1.74 min; ESI-MS(+) m/z [M+2H]²⁺: 1131.

### Preparation of Compound 2064

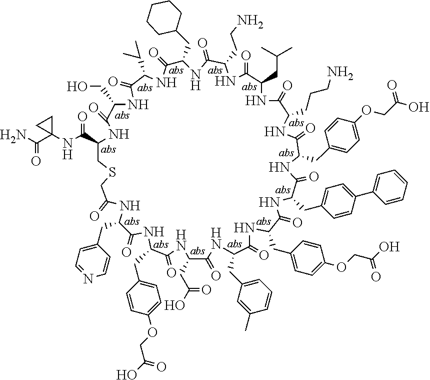 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)NC3(C(N)=O)CC3)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2064 was prepared on a 25 μmol scale. The yield of the product was 22.3 mg, and its estimated purity by LCMS analysis was 95.2%. Analysis condition A: Retention time=1.43 min; ESI-MS(+) m/z [M+3H]³⁺: 741.

### Preparation of Compound 2065

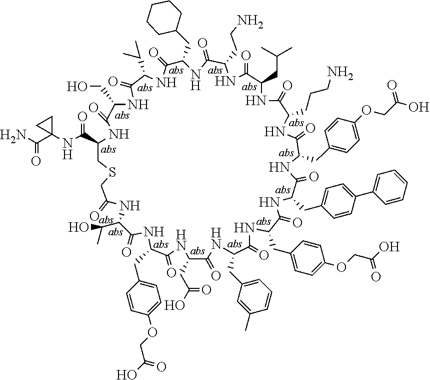 [SMILES: Cc1cccc(C[C@@H]2NC(=O)[C@H](CC(=O)O)NC(=O)[C@H](Cc3ccc(OCC(=O)O)cc3)NC(=O)[C@H]([C@@H](C)O)NC(=O)CSC[C@@H](C(=O)NC3(C(N)=O)CC3)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2065 was prepared on a 25 μmol scale. The yield of the product was 10.9 mg, and its estimated purity by LCMS analysis was 95.2%. Analysis condition A: Retention time=1.45 min; ESI-MS(+) m/z [M+2H]²⁺: 1088.

### Preparation of Compound 2066

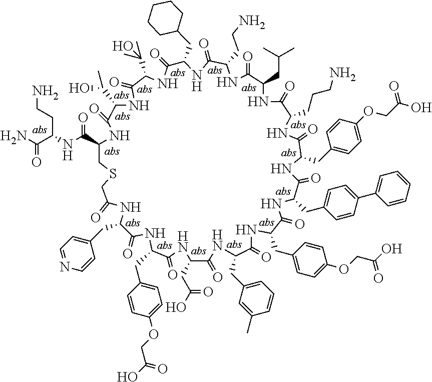 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H]([C@@H](C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2066 was prepared on a 25 μmol scale. The yield of the product was 4.9 mg, and its estimated purity by LCMS analysis was 97.7%. Analysis condition B: Retention time=1.54 min; ESI-MS(+) m/z [M+2H]²⁺: 1135.2.

### Preparation of Compound 2067

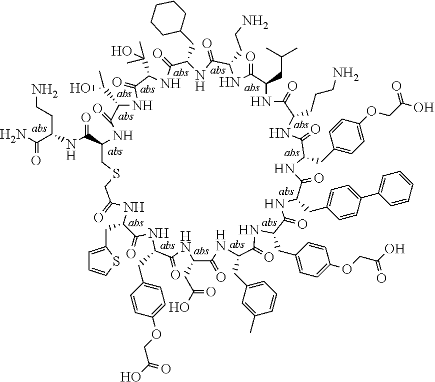 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cccs3)NC(=O)CSC[C@@H](C(=O)N[C@@H](CCN)C(N)=O)NC(=O)[C@H]([C@@H](C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2067 was prepared on a 25 μmol scale. The yield of the product was 6.7 mg, and its estimated purity by LCMS analysis was 98.4%. Analysis condition B: Retention time=1.65 min; ESI-MS(+) m/z [M+2H]²⁺: 1137.2.

### Preparation of Compound 2068

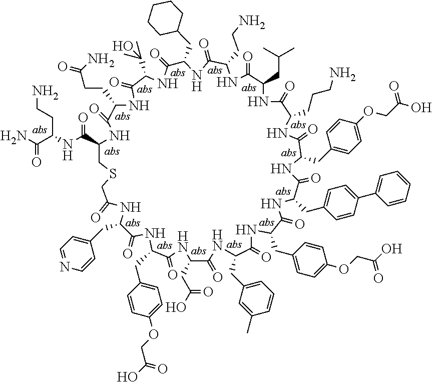 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CCC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2068 was prepared on a 25 μmol scale. The yield of the product was 8.8 mg, and its estimated purity by LCMS analysis was 95.2%. Analysis condition B: Retention time=1.47 min; ESI-MS(+) m/z [M+2H]²⁺: 1149.1.

### Preparation of Compound 2069

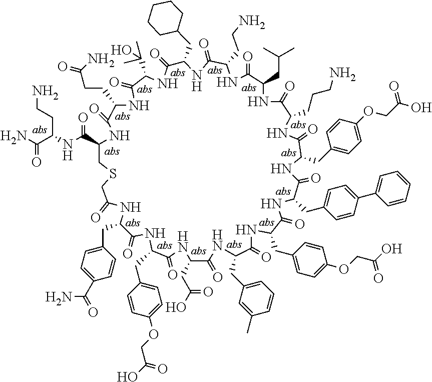 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(N)=O)cc3)NC(=O)CSC[C@@H](C(=O)N[C@@H](CCN)C(N)=O)NC(=O)[C@H](CCC(N)=O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2069 was prepared on a 25 μmol scale. The yield of the product was 5.7 mg, and its estimated purity by LCMS analysis was 96%. Analysis condition B: Retention time=1.56 min; ESI-MS(+) m/z [M+3H]³⁺: 780.

### Preparation of Compound 2070

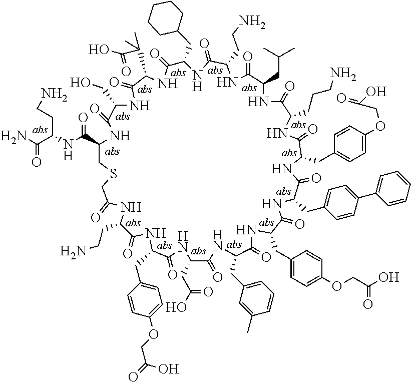 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](CO)NC(=O)[C@H](C(C)(C)C(=O)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2070 was prepared on a 50 μmol scale. The yield of the product was 9.2 mg, and its estimated purity by LCMS analysis was 92%. Analysis condition A: Retention time=1.44 min; ESI-MS(+) m/z [M+2H]²⁺: 1118.1.

### Preparation of Compound 2071

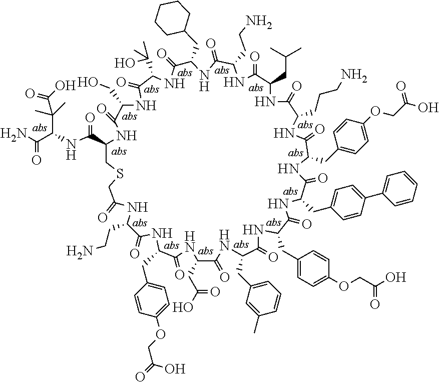 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@H](C(N)=O)C(C)(C)C(=O)O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2071 was prepared on a 50 μmol scale. The yield of the product was 5.5 mg, and its estimated purity by LCMS analysis was 98.2%. Analysis condition B: Retention time=1.64 min; ESI-MS(+) m/z [M+2H]²⁺: 1125.1.

### Preparation of Compound 2072

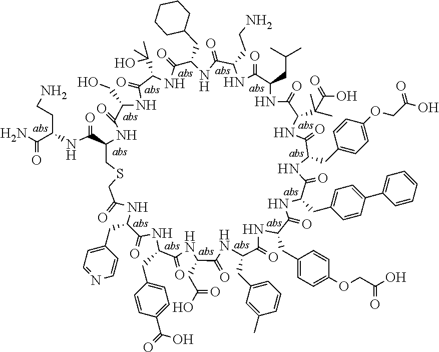 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C(C)(C)C(=O)O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2072 was prepared on a 50 μmol scale. The yield of the product was 35.3 mg, and its estimated purity by LCMS analysis was 92.3%. Analysis condition A: Retention time=1.4 min; ESI-MS(+) m/z [M+3H]³⁺: 752.1.

### Preparation of Compound 2073

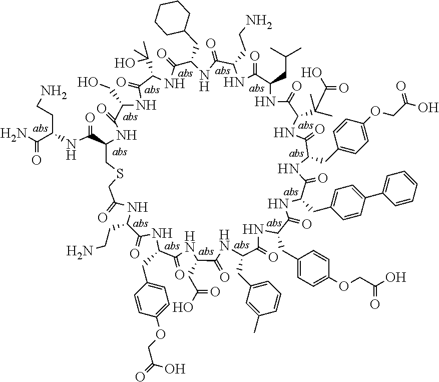 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](C(C)(C)C(=O)O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2073 was prepared on a 50 μmol scale. The yield of the product was 24.9 mg, and its estimated purity by LCMS analysis was 95.5%. Analysis condition A: Retention time=1.36 min; ESI-MS(+) m/z [M+2H]²⁺: 1118.1.

### Preparation of Compound 2074

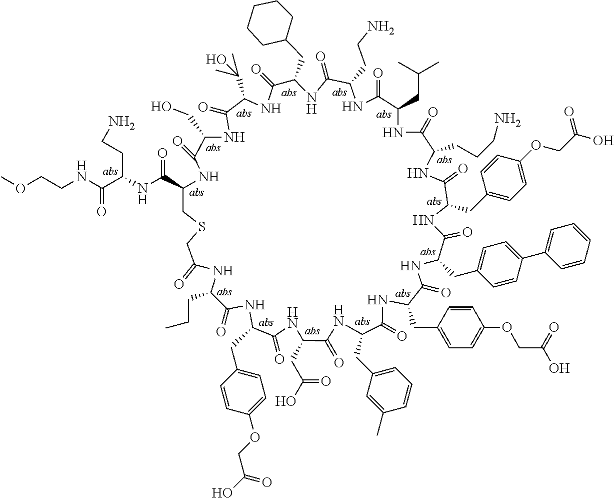 [SMILES: CCC[C@@H]1NC(=O)CSC[C@@H](C(=O)N[C@@H](CCN)C(=O)NCCOC)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2074 was prepared on a 25 μmol scale. The yield of the product was 10.6 mg, and its estimated purity by LCMS analysis was 100%. Analysis condition A: Retention time=1.47 min; ESI-MS(+) m/z [M+3H]³⁺: 755.

### Preparation of Compound 2075

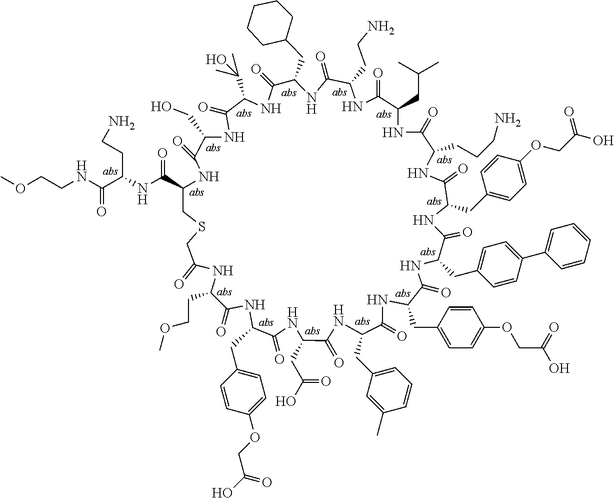 [SMILES: COCCNC(=O)[C@H](CCN)NC(=O)[C@@H]1CSCC(=O)N[C@@H](CCOC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1]

Compound 2075 was prepared on a 25 μmol scale. The yield of the product was 6.2 mg, and its estimated purity by LCMS analysis was 96.2%. Analysis condition B: Retention time=1.6 min; ESI-MS(+) m/z [M+2H]²⁺: 1140.

### Preparation of Compound 2076

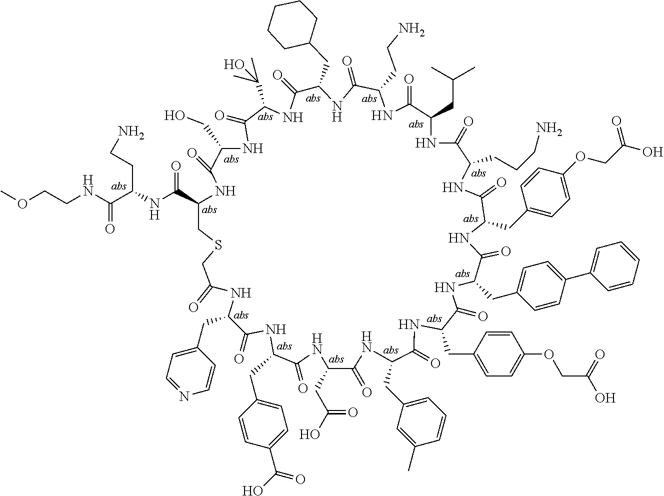 [SMILES: COCCNC(=O)[C@H](CCN)NC(=O)[C@@H]1CSCC(=O)N[C@@H](Cc2ccncc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1]

Compound 2076 was prepared on a 25 μmol scale. The yield of the product was 13.3 mg, and its estimated purity by LCMS analysis was 95.8%. Analysis condition B: Retention time=1.5 min; ESI-MS(+) m/z [M+2H]²⁺: 1142.1.

### Preparation of Compound 2077

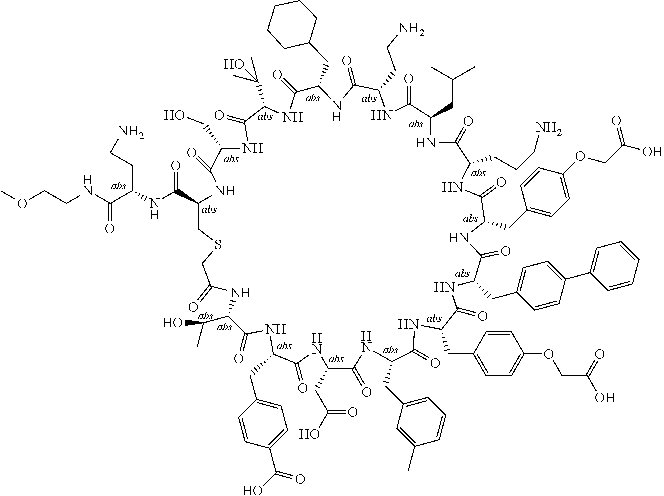 [SMILES: COCCNC(=O)[C@H](CCN)NC(=O)[C@@H]1CSCC(=O)N[C@@H]([C@@H](C)O)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1]

Compound 2077 was prepared on a 25 μmol scale. The yield of the product was 59.2 mg, and its estimated purity by LCMS analysis was 92.5%. Analysis condition A: Retention time=1.47 min; ESI-MS(+) m/z [M+2H]²⁺: 1118.1.

### Preparation of Compound 2078

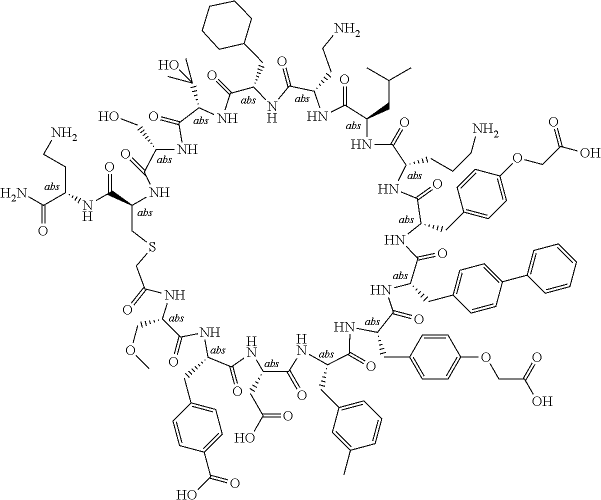 [SMILES: COC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2078 was prepared on a 25 μmol scale. The yield of the product was 7.8 mg, and its estimated purity by LCMS analysis was 92.3%. Analysis condition A: Retention time=1.42 min; ESI-MS(+) m/z [M+2H]²⁺: 1089.3.

### Preparation of Compound 2079

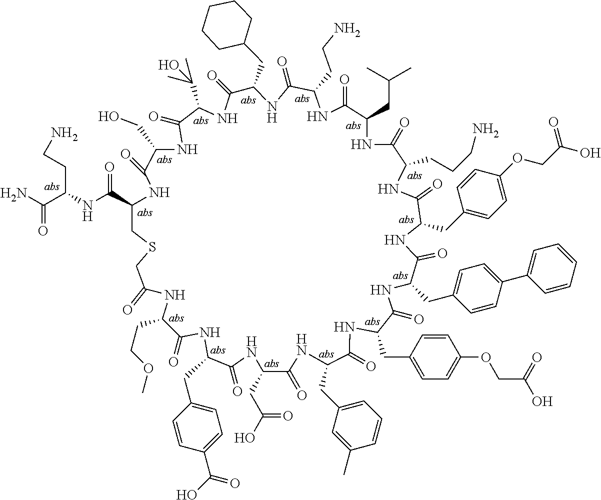 [SMILES: COCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2079 was prepared on a 25 μmol scale. The yield of the product was 3.7 mg, and its estimated purity by LCMS analysis was 93.1%. Analysis condition B: Retention time=1.57 min; ESI-MS(+) m/z [M+3H]³⁺: 731.

### Preparation of Compound 2080

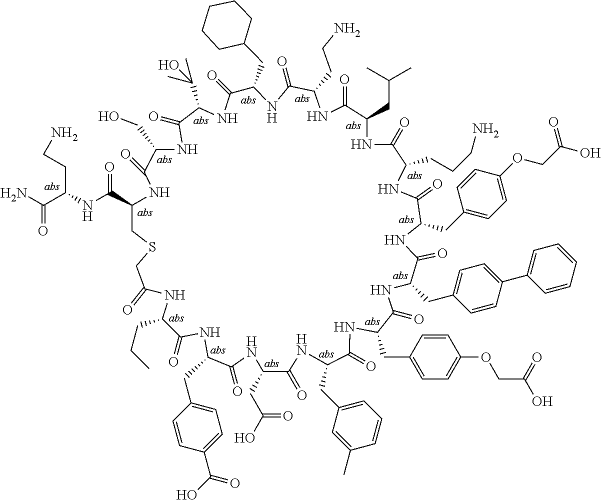 [SMILES: CCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC1=O]

Compound 2080 was prepared on a 25 μmol scale. The yield of the product was 9.1 mg, and its estimated purity by LCMS analysis was 95.6%. Analysis condition B: Retention time=1.62 min; ESI-MS(+) m/z [M+2H]²⁺: 1088.2.

### Preparation of Compound 2081

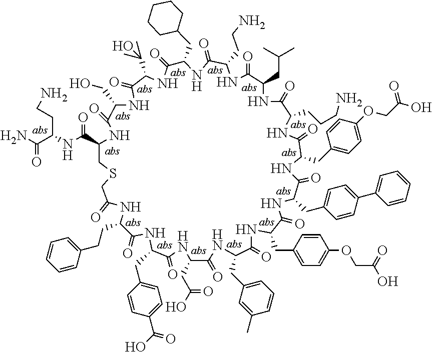 [SMILES: Cc1cccc(C[C@@H]2NC(=O)[C@H](CC(=O)O)NC(=O)[C@H](Cc3ccc(C(=O)O)cc3)NC(=O)[C@H](CCc3ccccc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2081 was prepared on a 25 μmol scale. The yield of the product was 1.1 mg, and its estimated purity by LCMS analysis was 100%. Analysis condition A: Retention time=1.48 min; ESI-MS(+) m/z [M+2H]²⁺: 1119.3.

### Preparation of Compound 2082

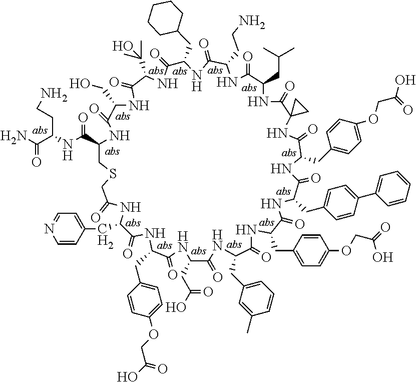 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)C3(CC3)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2082 was prepared on a 30 μmol scale. The yield of the product was 10.5 mg, and its estimated purity by LCMS analysis was 96.8%. Analysis condition A: Retention time=1.5 min; ESI-MS(+) m/z [M+2H]²⁺: 1112.

### Preparation of Compound 2083

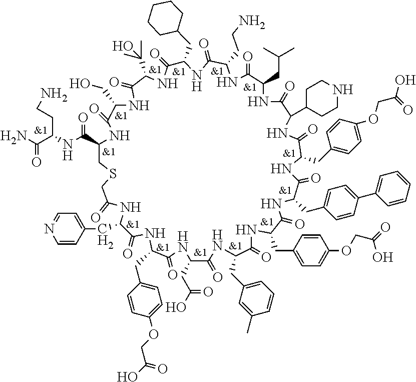 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)C(C3CCNCC3)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2083 was prepared on a 30 μmol scale. The yield of the product was 5.2 mg, and its estimated purity by LCMS analysis was 99.2%. Analysis condition B: Retention time=1.59 min; ESI-MS(+) m/z [M+3H]³⁺: 761.2.

### Preparation of Compound 2084

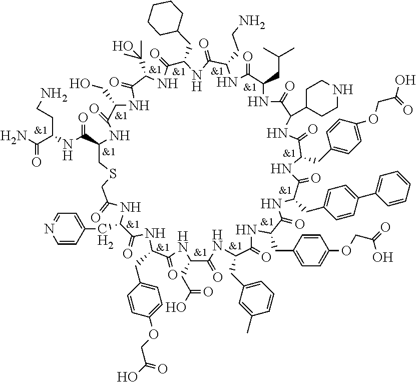 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)C(C3CCNCC3)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2084 was prepared on a 30 μmol scale. The yield of the product was 1.7 mg, and its estimated purity by LCMS analysis was 96.3%. Analysis condition B: Retention time=1.51 min; ESI-MS(+) m/z [M+2H]²⁺: 1141.1.

### Preparation of Compound 2085

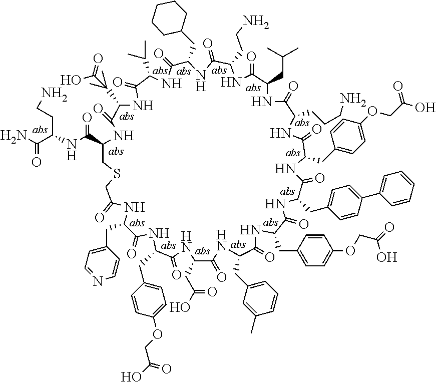 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](C(C)(C)C(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2085 was prepared on a 50 μmol scale. The yield of the product was 11 mg, and its estimated purity by LCMS analysis was 91.6%. Analysis condition B: Retention time=1.55 min; ESI-MS(+) m/z [M+2H]²⁺: 1147.2.

### Preparation of Compound 2086

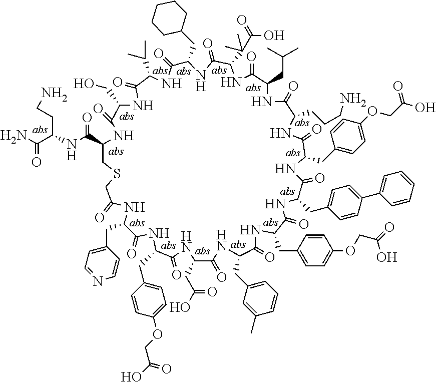 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)C)NC(=O)[C@H](CC3CCCCC3)NC(=O)[C@H](C(C)(C)C(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2086 was prepared on a 50 μmol scale. The yield of the product was 14 mg, and its estimated purity by LCMS analysis was 97.7%. Analysis condition B: Retention time=1.66 min; ESI-MS(+) m/z [M+2H]²⁺: 1141.3.

### Preparation of Compound 2087

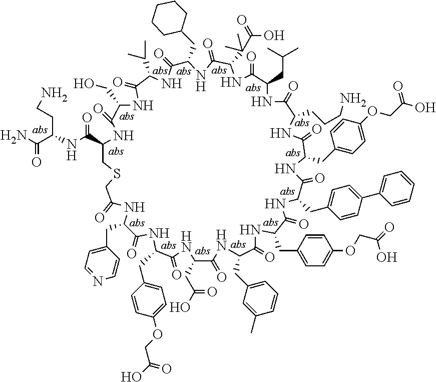 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)C)NC(=O)[C@H](CC3CCCCC3)NC(=O)[C@H](C(C)(C)C(=O)O)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2087 was prepared on a 50 μmol scale. The yield of the product was 9.5 mg, and its estimated purity by LCMS analysis was 91.6%. Analysis condition A: Retention time=1.37 min; ESI-MS(+) m/z [M+3H]³⁺: 761.1.

### Preparation of Compound 2088

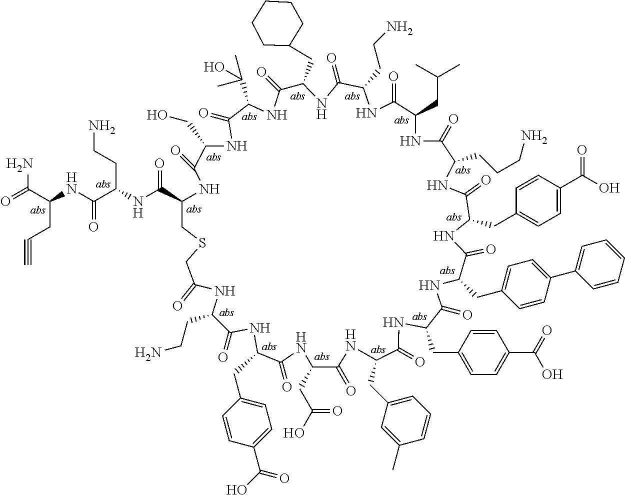 [SMILES: C#CC[C@H](NC(=O)[C@H](CCN)NC(=O)[C@@H]1CSCC(=O)N[C@@H](CCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2088 was prepared on a 50 μmol scale. The yield of the product was 11.5 mg, and its estimated purity by LCMS analysis was 92.1%. Analysis condition A: Retention time=1.52 min; ESI-MS(+) m/z [M+3H]³⁺: 738.2.

### Preparation of Compound 2089

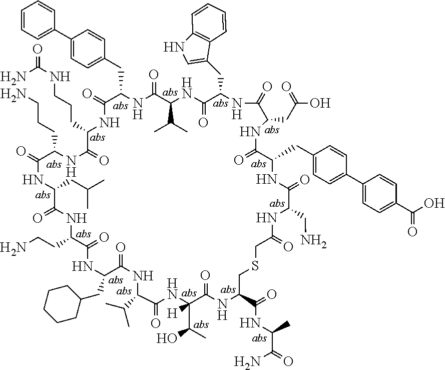 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3ccc(C(=O)O)cc3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2089 was prepared on a 23 μmol scale. The yield of the product was 5.6 mg, and its estimated purity by LCMS analysis was 92.8%. Analysis condition B: Retention time=1.54 min; ESI-MS(+) m/z [M+2H]²⁺: 1024.

### Preparation of Compound 2090

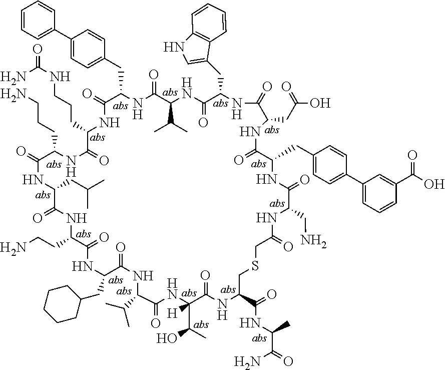 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2090 was prepared on a 23 μmol scale. The yield of the product was 8 mg, and its estimated purity by LCMS analysis was 100%. Analysis condition A: Retention time=1.61 min; ESI-MS(+) m/z [M+2H]²⁺: 1024.3.

### Preparation of Compound 2091

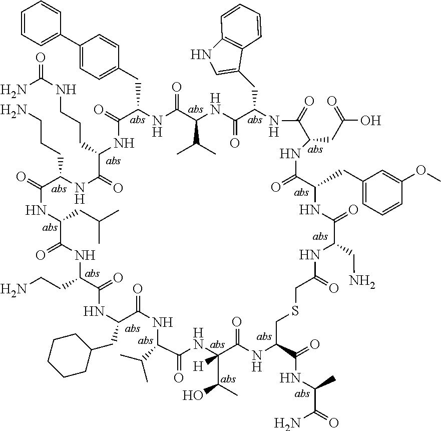 [SMILES: COc1cccc(C[C@@H]2NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)c1]

Compound 2091 was prepared on a 23 μmol scale. The yield of the product was 3.4 mg, and its estimated purity by LCMS analysis was 96.9%. Analysis condition B: Retention time=1.47 min; ESI-MS(+) m/z [M+2H]²⁺: 979.

### Preparation of Compound 2092

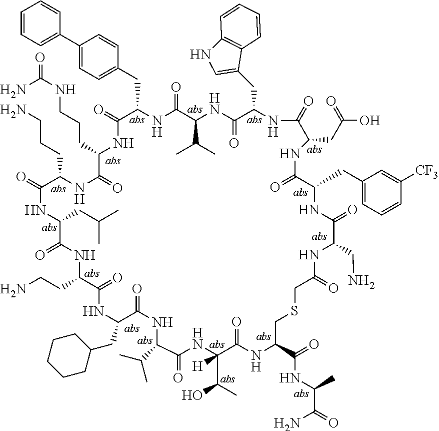 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(C(F)(F)F)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2092 was prepared on a 23 μmol scale. The yield of the product was 1.2 mg, and its estimated purity by LCMS analysis was 95.5%. Analysis condition B: Retention time=1.69 min; ESI-MS(+) m/z [M+3H]³⁺: 665.1.

### Preparation of Compound 2093

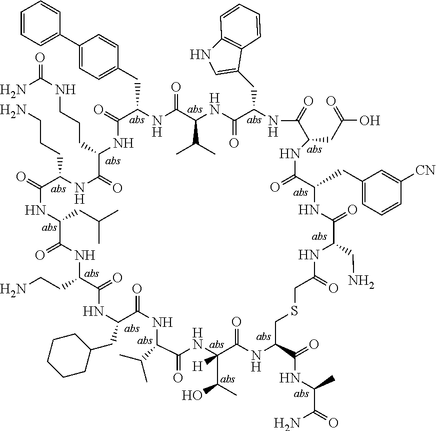 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(C#N)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2093 was prepared on a 23 μmol scale. The yield of the product was 11.6 mg, and its estimated purity by LCMS analysis was 90.7%. Analysis condition B: Retention time=1.54, 1.57 min; ESI-MS(+) m/z [M+H]⁺: 1951.13, 1951.13.

### Preparation of Compound 2094

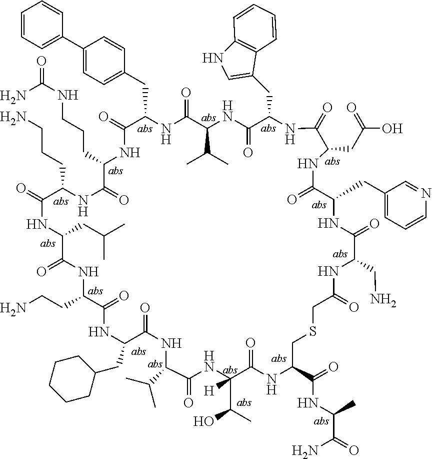 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2094 was prepared on a 23 μmol scale. The yield of the product was 2.2 mg, and its estimated purity by LCMS analysis was 97%. Analysis condition B: Retention time=1.43 min; ESI-MS(+) m/z [M+H]⁺: 1927.

### Preparation of Compound 2095

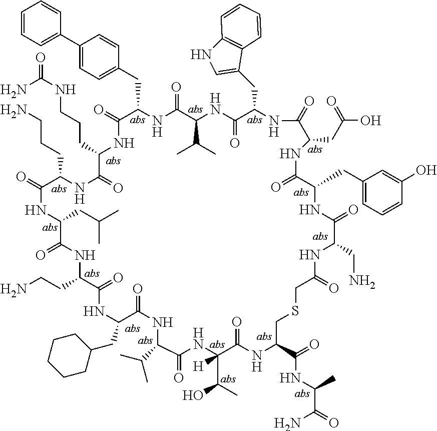 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(O)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2095 was prepared on a 23 μmol scale. The yield of the product was 2.6 mg, and its estimated purity by LCMS analysis was 93.1%. Analysis condition A: Retention time=1.5 min; ESI-MS(+) m/z [M+2H]²⁺: 971.9.

### Preparation of Compound 2096

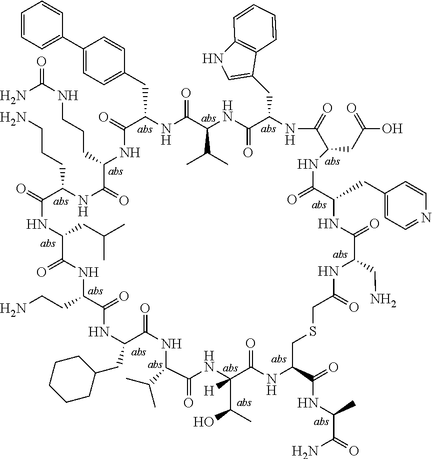 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2096 was prepared on a 23 μmol scale. The yield of the product was 6.7 mg, and its estimated purity by LCMS analysis was 100%. Analysis condition A: Retention time=1.62 min; ESI-MS(+) m/z [M+H]⁺: 1927.

### Preparation of Compound 2097

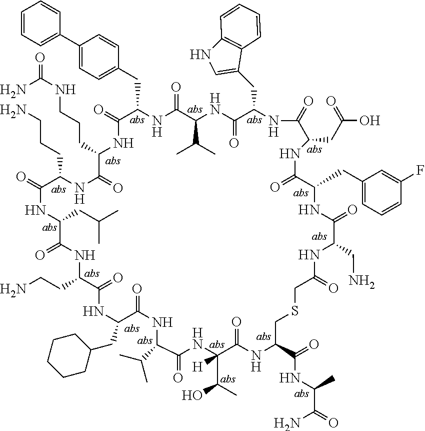 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(F)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2097 was prepared on a 22.7 μmol scale. The yield of the product was 6.5 mg, and its estimated purity by LCMS analysis was 95.4%. Analysis condition B: Retention time=1.49 min; ESI-MS(+) m/z [M+2H]²⁺: 973.2.

### Preparation of Compound 2098

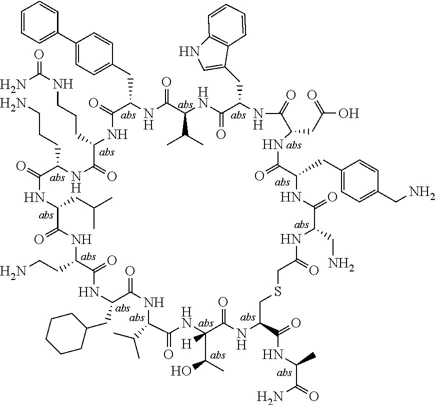 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(CN)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2098 was prepared on a 23 μmol scale. The yield of the product was 6.2 mg, and its estimated purity by LCMS analysis was 87.3%. Analysis condition B: Retention time=1.31 min; ESI-MS(+) m/z [M+3H]³⁺: 652.8.

### Preparation of Compound 2099

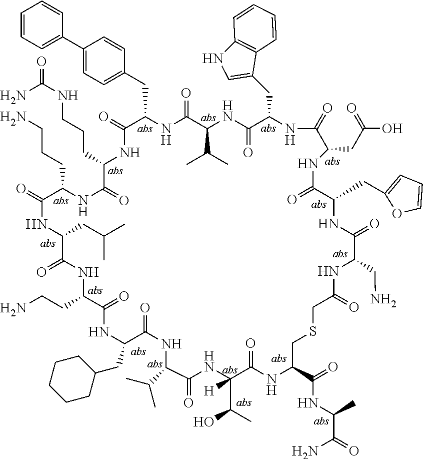 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccco2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2099 was prepared on a 22.7 μmol scale. The yield of the product was 8 mg, and its estimated purity by LCMS analysis was 100%. Analysis condition A: Retention time=1.79 min; ESI-MS(+) m/z [M+H]⁺: 1916.2.

### Preparation of Compound 2100

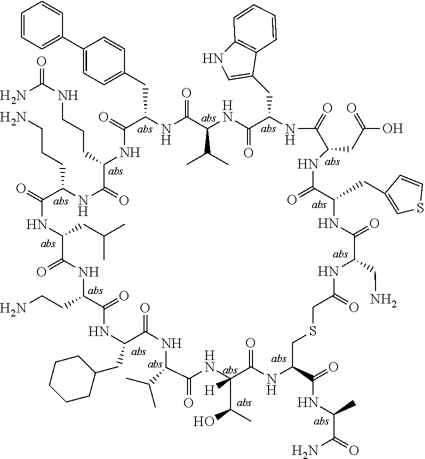 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccsc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2100 was prepared on a 23 μmol scale. The yield of the product was 2.7 mg, and its estimated purity by LCMS analysis was 94.6%. Analysis condition B: Retention time=1.48 min; ESI-MS(+) m/z [M+2H]²⁺: 967.2.

### Preparation of Compound 2101

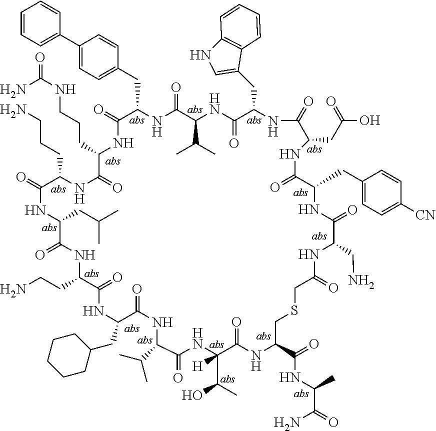 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2101 was prepared on a 23 μmol scale. The yield of the product was 9.6 mg, and its estimated purity by LCMS analysis was 100%. Analysis condition A: Retention time=1.81 min; ESI-MS(+) m/z [M+H]⁺: 1951.2.

### Preparation of Compound 2102

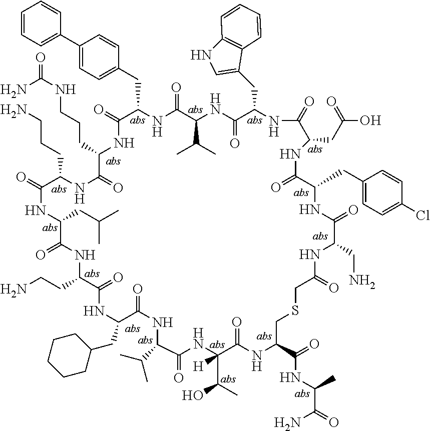 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(Cl)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2102 was prepared on a 23 μmol scale. The yield of the product was 12.1 mg, and its estimated purity by LCMS analysis was 89.6%. Analysis condition A: Retention time=1.94 min; ESI-MS(+) m/z [M+H]⁺: 1960.2.

### Preparation of Compound 2103

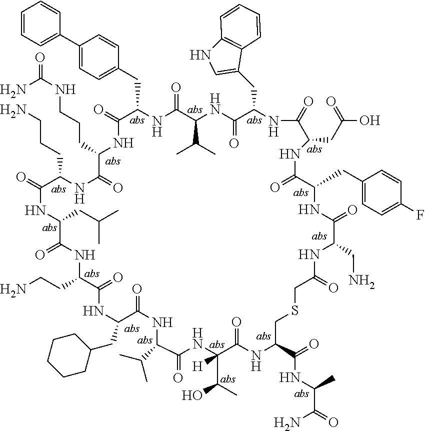 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(F)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2103 was prepared on a 23 μmol scale. The yield of the product was 5.2 mg, and its estimated purity by LCMS analysis was 100%. Analysis condition A: Retention time=1.87 min; ESI-MS(+) m/z [M+H]⁺: 1944.1.

### Preparation of Compound 2104

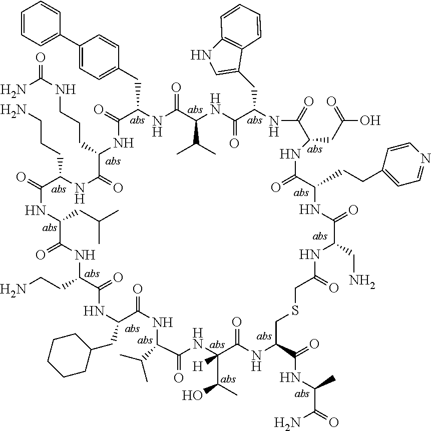 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2104 was prepared on a 23 μmol scale. The yield of the product was 7.9 mg, and its estimated purity by LCMS analysis was 84.1%. Analysis condition B: Retention time=1.79 min; ESI-MS(+) m/z [M+3H]³⁺: 647.9.

### Preparation of Compound 2105

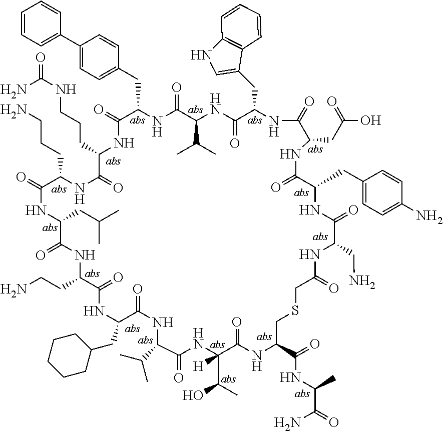 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2105 was prepared on a 23 μmol scale. The yield of the product was 5.9 mg, and its estimated purity by LCMS analysis was 96.9%. Analysis condition B: Retention time=1.41 min; ESI-MS(+) m/z [M+H]⁺: 1940.7.

### Preparation of Compound 2106

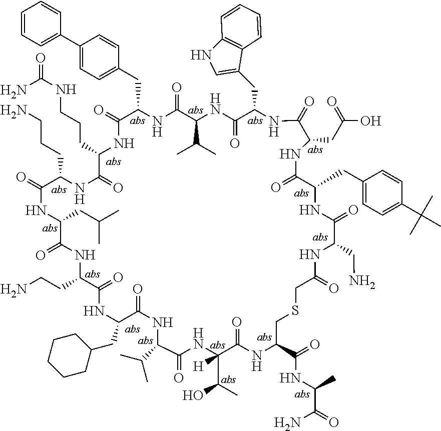 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(C)(C)C)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2106 was prepared on a 23 μmol scale. The yield of the product was 2 mg, and its estimated purity by LCMS analysis was 100%. Analysis condition A: Retention time=2 min; ESI-MS(+) m/z [M+H]⁺: 1982.

### Preparation of Compound 2107

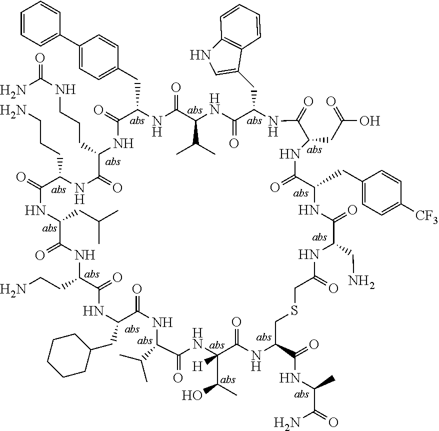 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(F)(F)F)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2107 was prepared on a 23 μmol scale. The yield of the product was 8.4 mg, and its estimated purity by LCMS analysis was 93.7%. Analysis condition A: Retention time=2.38 min; ESI-MS(+) m/z [M+2H]²⁺: 998.1.

### Preparation of Compound 2108

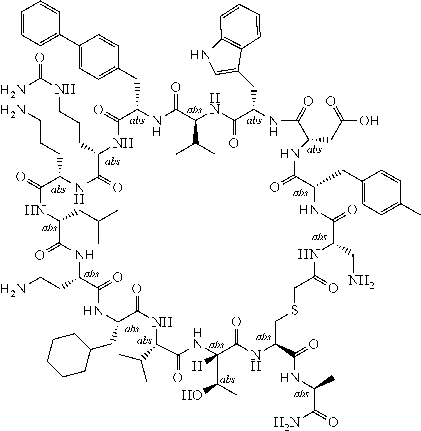 [SMILES: Cc1ccc(C[C@@H]2NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)cc1]

Compound 2108 was prepared on a 23 μmol scale. The yield of the product was 5.2 mg, and its estimated purity by LCMS analysis was 91.6%. Analysis condition B: Retention time=1.6 min; ESI-MS(+) m/z [M+H]⁺: 1940.

### Preparation of Compound 2109

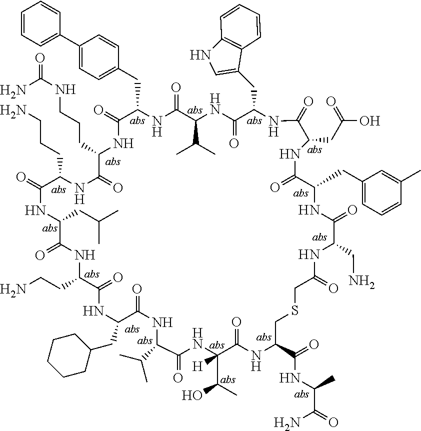 [SMILES: Cc1cccc(C[C@@H]2NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)c1]

Compound 2109 was prepared on a 23 μmol scale. The yield of the product was 1.5 mg, and its estimated purity by LCMS analysis was 91.7%. Analysis condition B: Retention time=1.55 min; ESI-MS(+) m/z [M+3H]³⁺: 647.8.

### Preparation of Compound 2110

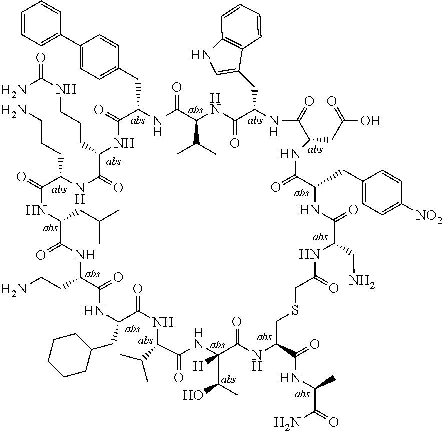 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc([N+](=O)[O-])cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2110 was prepared on a 23 μmol scale. The yield of the product was 7.4 mg, and its estimated purity by LCMS analysis was 96.4%. Analysis condition B: Retention time=1.58 min; ESI-MS(+) m/z [M+H]⁺: 1970.7.

### Preparation of Compound 2111

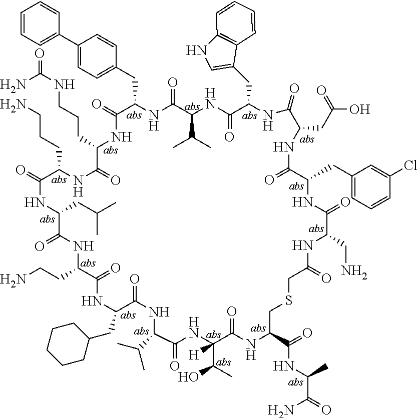 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(Cl)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2111 was prepared on a 23 μmol scale. The yield of the product was 7.1 mg, and its estimated purity by LCMS analysis was 100%. Analysis condition A: Retention time=1.94 min; ESI-MS(+) m/z [M+H]⁺: 1960.1.

### Preparation of Compound 2112

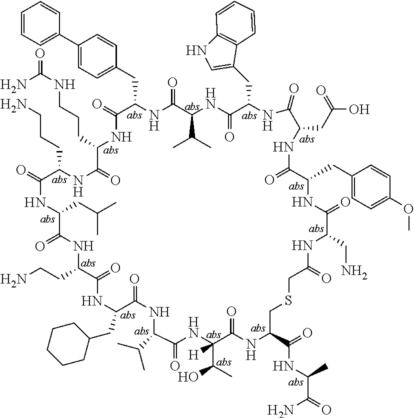 [SMILES: COc1ccc(C[C@@H]2NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)cc1]

Compound 2112 was prepared on a 23 μmol scale. The yield of the product was 6 mg, and its estimated purity by LCMS analysis was 94.6%. Analysis condition B: Retention time=1.42 min; ESI-MS(+) m/z [M+2H]²⁺: 979.1.

### Preparation of Compound 2113

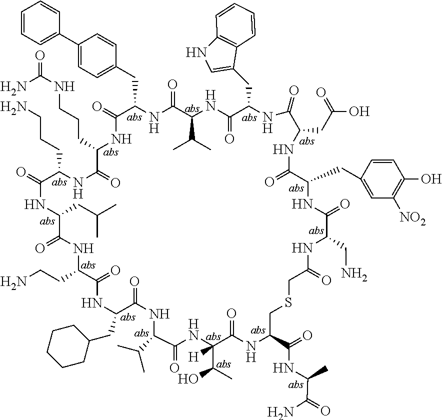 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)c([N+](=O)[O-])c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2113 was prepared on a 23 μmol scale. The yield of the product was 2.6 mg, and its estimated purity by LCMS analysis was 90.7%. Analysis condition B: Retention time=1.56 min; ESI-MS(+) m/z [M+H]⁺: 1986.7.

### Preparation of Compound 2114

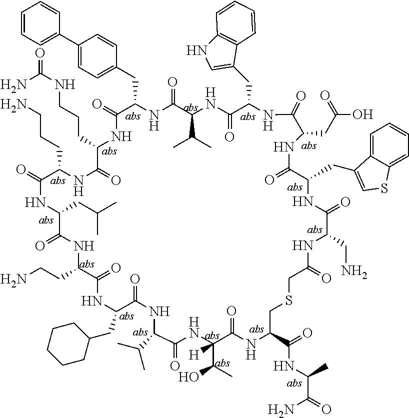 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2csc3ccccc23)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2114 was prepared on a 23 μmol scale. The yield of the product was 4.4 mg, and its estimated purity by LCMS analysis was 84.2%. Analysis condition A: Retention time=1.96 min; ESI-MS(+) m/z [M+H]⁺: 1982.2.

### Preparation of Compound 2115

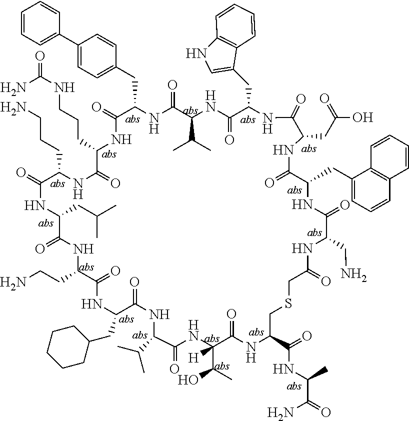 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc3ccccc23)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2115 was prepared on a 23 μmol scale. The yield of the product was 5.6 mg, and its estimated purity by LCMS analysis was 85%. Analysis condition A: Retention time=1.97 min; ESI-MS(+) m/z [M+H]⁺: 1976.3.

### Preparation of Compound 2116

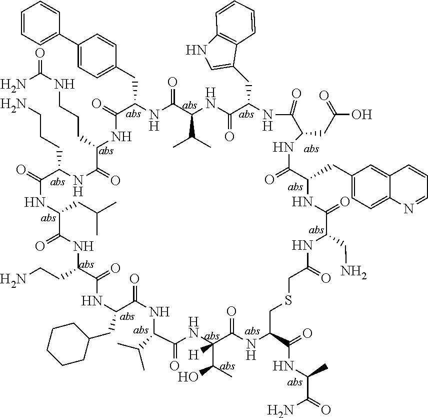 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2116 was prepared on a 23 μmol scale. The yield of the product was 3.5 mg, and its estimated purity by LCMS analysis was 100%. Analysis condition A: Retention time=1.75 min; ESI-MS(+) m/z [M+H]⁺: 1977.1.

### Preparation of Compound 2117

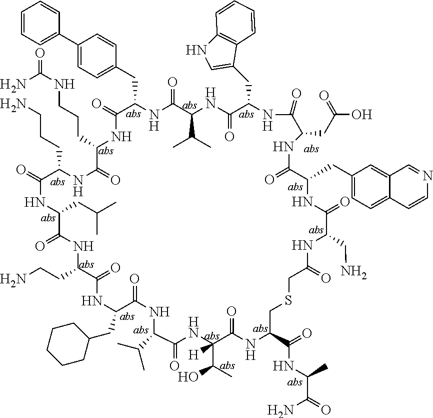 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ccncc3c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2117 was prepared on a 23 μmol scale. The yield of the product was 4.9 mg, and its estimated purity by LCMS analysis was 95.5%. Analysis condition B: Retention time=1.35 min; ESI-MS(+) m/z [M+H]⁺: 1977.2.

### Preparation of Compound 2118

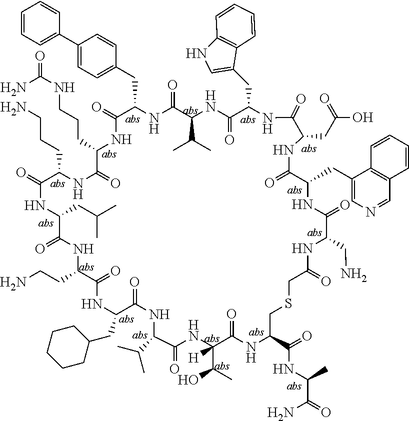 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cncc3ccccc23)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2118 was prepared on a 23 μmol scale. The yield of the product was 6.6 mg, and its estimated purity by LCMS analysis was 94.8%. Analysis condition A: Retention time=1.68 min; ESI-MS(+) m/z [M+H]⁺: 1976.7.

### Preparation of Compound 2119

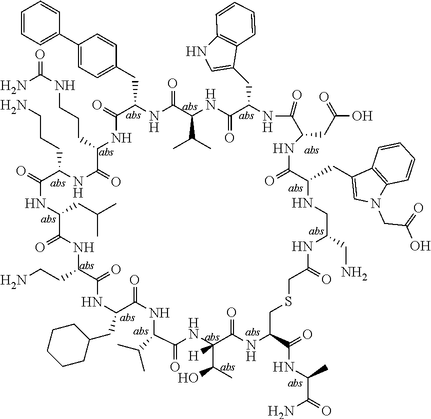 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cn(CC(=O)O)c3ccccc23)NC[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2119 was prepared on a 23 μmol scale. The yield of the product was 5.1 mg, and its estimated purity by LCMS analysis was 100%. Analysis condition A: Retention time=1.71 min; ESI-MS(+) m/z [M+3H]³⁺: 675.2.

### Preparation of Compound 2120

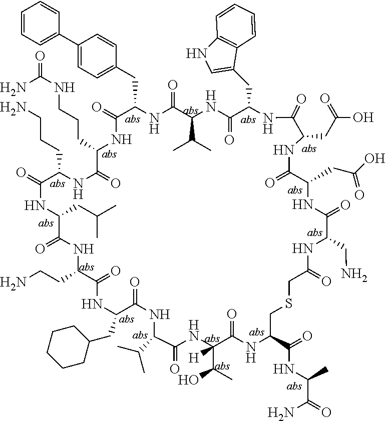 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CC(=O)O)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2120 was prepared on a 23 μmol scale. The yield of the product was 4.3 mg, and its estimated purity by LCMS analysis was 100%. Analysis condition A: Retention time=1.63 min; ESI-MS(+) m/z [M+2H]²⁺: 948.

### Preparation of Compound 2121

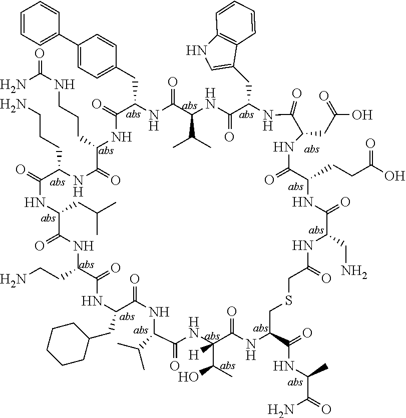 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCC(=O)O)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2121 was prepared on a 23 μmol scale. The yield of the product was 3.6 mg, and its estimated purity by LCMS analysis was 100%. Analysis condition A: Retention time=1.62 min; ESI-MS(+) m/z [M+H]⁺: 1908.1.

### Preparation of Compound 2122

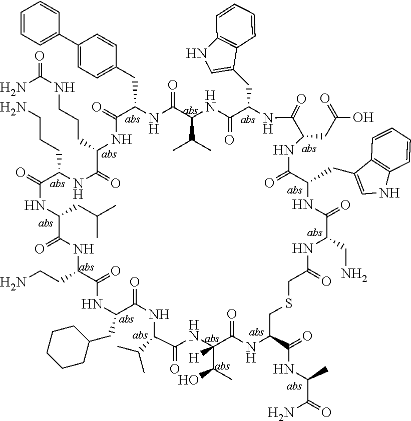 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2c[nH]c3ccccc23)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2122 was prepared on a 23 μmol scale. The yield of the product was 8.3 mg, and its estimated purity by LCMS analysis was 96.7%. Analysis condition A: Retention time=1.85 min; ESI-MS(+) m/z [M+H]⁺: 1965.3.

### Preparation of Compound 2123

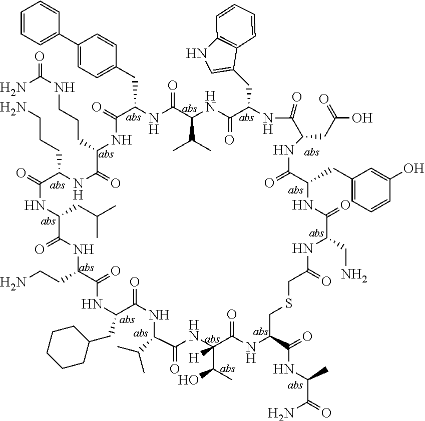 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccc(O)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2123 was prepared on a 23 μmol scale. The yield of the product was 1.7 mg, and its estimated purity by LCMS analysis was 93.1%. Analysis condition A: Retention time=1.5 min; ESI-MS(+) m/z [M+2H]²⁺: 971.9.

### Preparation of Compound 2124

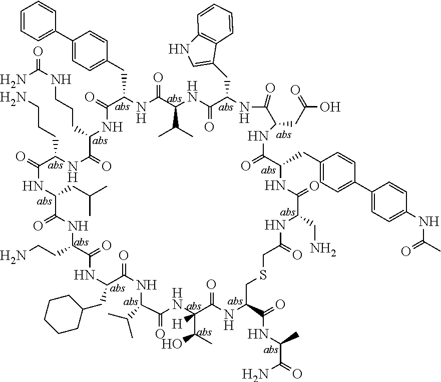 [SMILES: CC(=O)Nc1ccc(-c2ccc(C[C@@H]3NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC4CCCCC4)NC(=O)[C@H](CCN)NC(=O)[C@@H](CC(C)C)NC(=O)[C@H](CCCN)NC(=O)[C@H](CCCNC(N)=O)NC(=O)[C@H](Cc4ccc(-c5ccccc5)cc4)NC(=O)[C@H](C(C)C)NC(=O)[C@H](Cc4c[nH]c5ccccc45)NC(=O)[C@H](CC(=O)O)NC3=O)cc2)cc1]

Compound 2124 was prepared on a 23 μmol scale. The yield of the product was 2.4 mg, and its estimated purity by LCMS analysis was 96.1%. Analysis condition B: Retention time=1.46 min; ESI-MS(+) m/z [M+3H]³⁺: 687.6.

### Preparation of Compound 2125

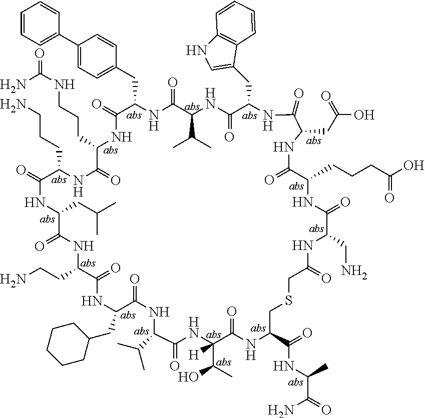 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCCC(=O)O)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2125 was prepared on a 23 μmol scale. The yield of the product was 4.9 mg, and its estimated purity by LCMS analysis was 100%. Analysis condition A: Retention time=1.62 min; ESI-MS(+) m/z [M+H]⁺: 1923.

### Preparation of Compound 2126

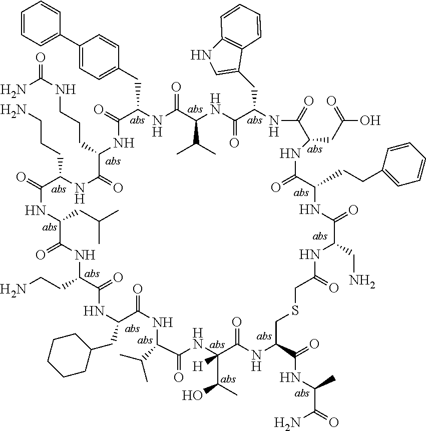 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CCc2ccccc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2126 was prepared on a 23 μmol scale. The yield of the product was 6.7 mg, and its estimated purity by LCMS analysis was 100%. Analysis condition A: Retention time=1.93 min; ESI-MS(+) m/z [M+H]⁺: 1940.1.

### Preparation of Compound 2127

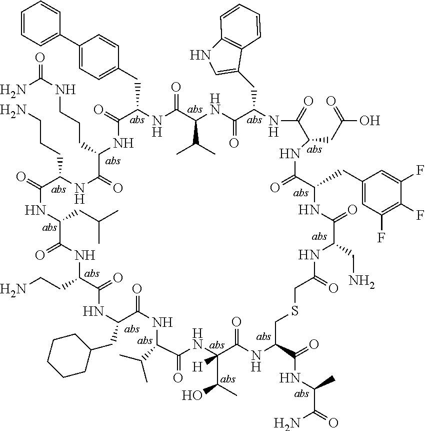 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cc(F)c(F)c(F)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2127 was prepared on a 23 μmol scale. The yield of the product was 7 mg, and its estimated purity by LCMS analysis was 100%. Analysis condition A: Retention time=1.97 min; ESI-MS(+) m/z [M+H]⁺: 1981.3.

### Preparation of Compound 2128

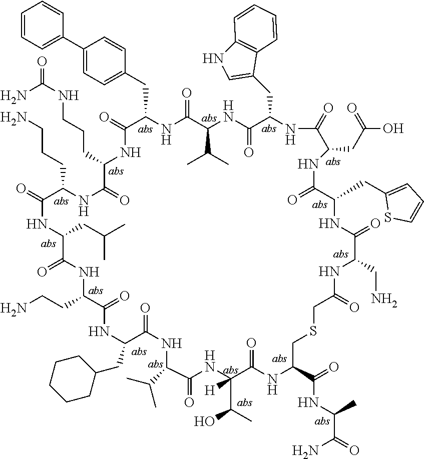 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2128 was prepared on a 23 μmol scale. The yield of the product was 2.3 mg, and its estimated purity by LCMS analysis was 98.9%. Analysis condition B: Retention time=1.49 min; ESI-MS(+) m/z [M+3H]³⁺: 645.2.

### Preparation of Compound 2129

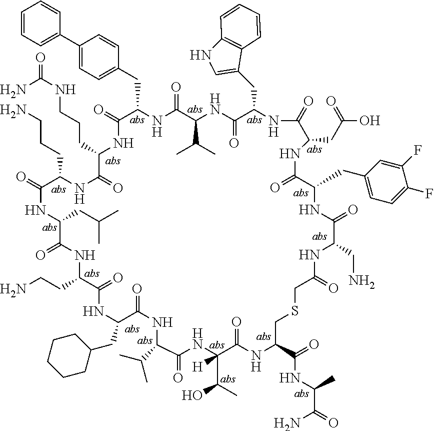 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(F)c(F)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2129 was prepared on a 23 μmol scale. The yield of the product was 7.2 mg, and its estimated purity by LCMS analysis was 100%. Analysis condition A: Retention time=1.93 min; ESI-MS(+) m/z [M+H]⁺: 1962.2.

### Preparation of Compound 2130

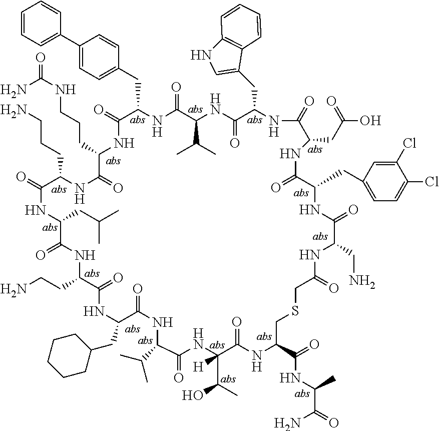 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(Cl)c(Cl)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2130 was prepared on a 23 μmol scale. The yield of the product was 3.9 mg, and its estimated purity by LCMS analysis was 92.1%. Analysis condition B: Retention time=1.58, 1.61 min; ESI-MS(+) m/z [M+3H]³⁺: 666.25, 666.06.

### Preparation of Compound 2131

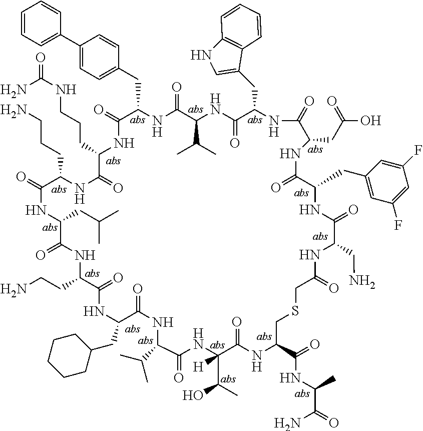 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cc(F)cc(F)c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2131 was prepared on a 23 μmol scale. The yield of the product was 9.8 mg, and its estimated purity by LCMS analysis was 90.4%. Analysis condition B: Retention time=1.51 min; ESI-MS(+) m/z [M+3H]³⁺: 655.1.

### Preparation of Compound 2132

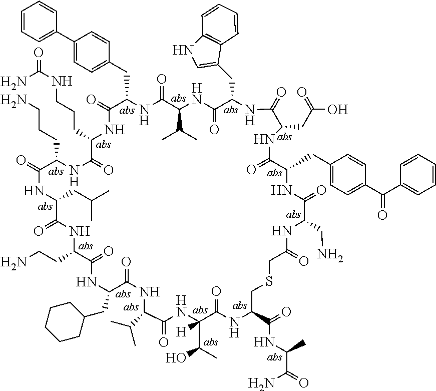 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)c3ccccc3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2132 was prepared on a 23 μmol scale. The yield of the product was 4 mg, and its estimated purity by LCMS analysis was 100%. Analysis condition A: Retention time=1.99 min; ESI-MS(+) m/z [M+2H]²⁺: 1015.5.

### Preparation of Compound 2133

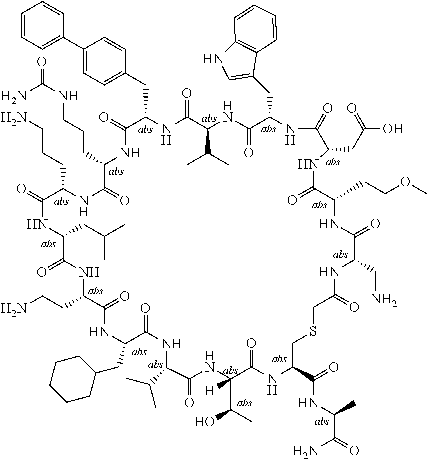 [SMILES: COCC[C@@H]1NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC1=O]

Compound 2133 was prepared on a 23 μmol scale. The yield of the product was 3.9 mg, and its estimated purity by LCMS analysis was 95.8%. Analysis condition A: Retention time=1.96, 2.12 min; ESI-MS(+) m/z [M+2H]²⁺: 948.04, 948.22.

### Preparation of Compound 2134

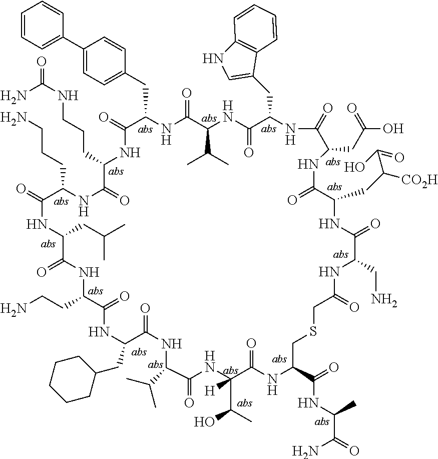 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](CC(C(=O)O)C(=O)O)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2134 was prepared on a 23 μmol scale. The yield of the product was 2.4 mg, and its estimated purity by LCMS analysis was 93.6%. Analysis condition B: Retention time=1.43 min; ESI-MS(+) m/z [M+3H]³⁺: 651.5.

### Preparation of Compound 2135

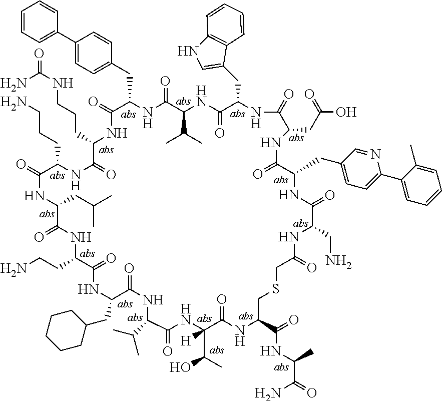 [SMILES: Cc1ccccc1-c1ccc(C[C@@H]2NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC(=O)[C@H](Cc3c[nH]c4ccccc34)NC(=O)[C@H](CC(=O)O)NC2=O)cn1]

Compound 2135 was prepared on a 23 μmol scale. The yield of the product was 1.4 mg, and its estimated purity by LCMS analysis was 100%. Analysis condition A: Retention time=1.81 min; ESI-MS(+) m/z [M+2H]²⁺: 1009.4.

### Preparation of Compound 2136

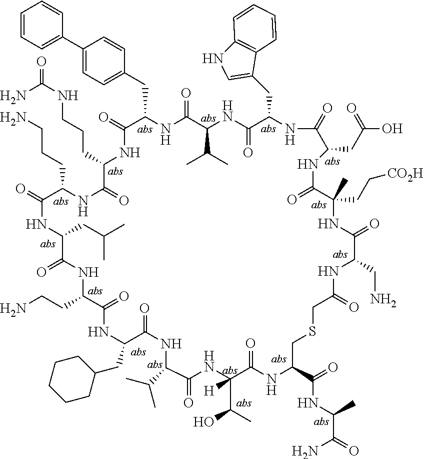 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@](C)(CCC(=O)O)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2136 was prepared on a 23 μmol scale. The yield of the product was 0.9 mg, and its estimated purity by LCMS analysis was 100%. Analysis condition A: Retention time=1.56 min; ESI-MS(+) m/z [M+2H]²⁺: 962.1.

### Preparation of Compound 2137

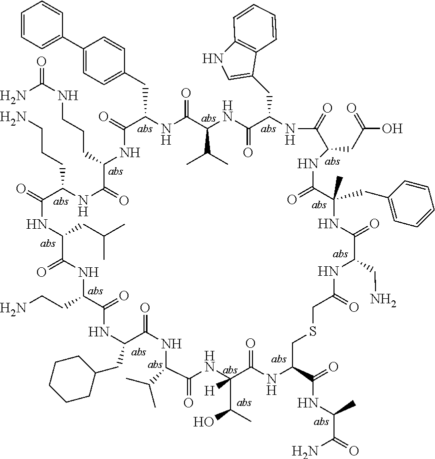 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@](C)(Cc2ccccc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2137 was prepared on a 23 μmol scale. The yield of the product was 3.6 mg, and its estimated purity by LCMS analysis was 91.5%. Analysis condition A: Retention time=2.24, 2.49 min; ESI-MS(+) m/z [M+2H]²⁺: 971.04, 971.02.

### Preparation of Compound 2138

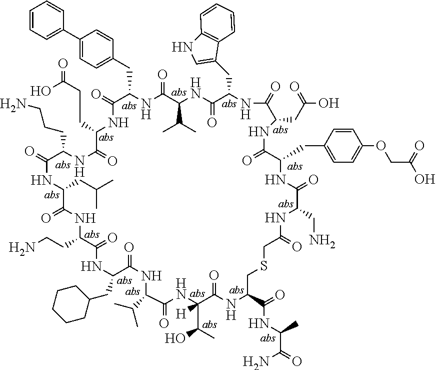 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2138 was prepared on a 23 μmol scale. The yield of the product was 3.6 mg, and its estimated purity by LCMS analysis was 98.3%. Analysis condition A: Retention time=1.48 min; ESI-MS(+) m/z [M+2H]²⁺: 996.1.

### Preparation of Compound 2139

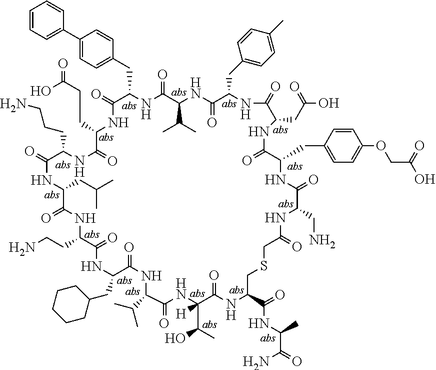 [SMILES: Cc1ccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)cc1]

Compound 2139 was prepared on a 23 μmol scale. The yield of the product was 1.1 mg, and its estimated purity by LCMS analysis was 100%. Analysis condition A: Retention time=1.57 min; ESI-MS(+) m/z [M+H]⁺: 1946.7.

### Preparation of Compound 2140

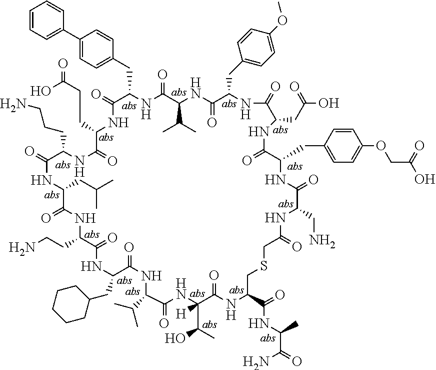 [SMILES: COc1ccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)cc1]

Compound 2140 was prepared on a 23 μmol scale. The yield of the product was 3 mg, and its estimated purity by LCMS analysis was 91.3%. Analysis condition B: Retention time=1.43, 1.48 min; ESI-MS(+) m/z [M+H]⁺: 1964.

### Preparation of Compound 2141

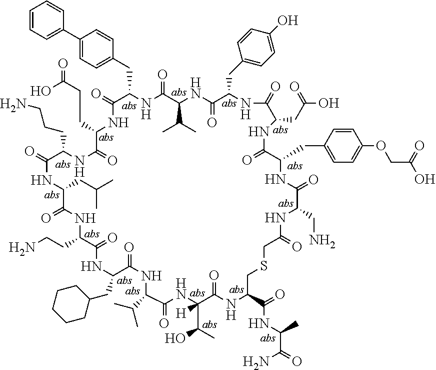 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc(O)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2141 was prepared on a 23 μmol scale. The yield of the product was 3 mg, and its estimated purity by LCMS analysis was 100%. Analysis condition A: Retention time=1.45 min; ESI-MS(+) m/z [M+H]⁺: 1948.9.

### Preparation of Compound 2142

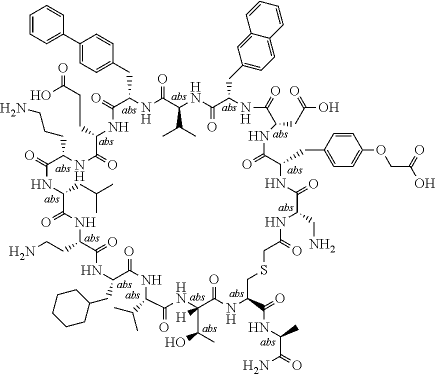 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc3ccccc3c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2142 was prepared on a 23 μmol scale. The yield of the product was 1.6 mg, and its estimated purity by LCMS analysis was 93.9%. Analysis condition A: Retention time=1.52 min; ESI-MS(+) m/z [M+3H]³⁺: 662.1.

### Preparation of Compound 2143

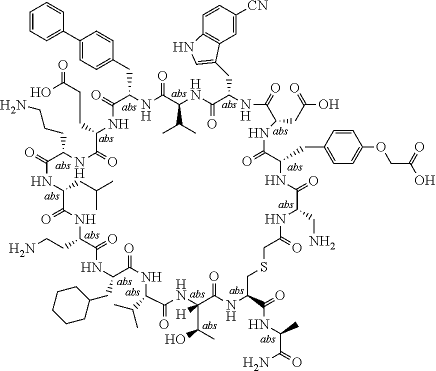 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccc(C#N)cc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2143 was prepared on a 23 μmol scale. The yield of the product was 2.6 mg, and its estimated purity by LCMS analysis was 96.5%. Analysis condition B: Retention time=1.49 min; ESI-MS(+) m/z [M+H]⁺: 1996.9.

### Preparation of Compound 2144

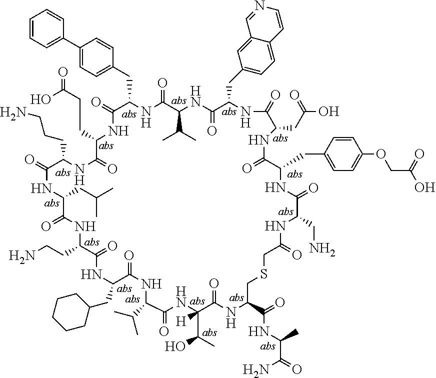 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc3ccncc3c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2144 was prepared on a 23 μmol scale. The yield of the product was 1.1 mg, and its estimated purity by LCMS analysis was 98.2%. Analysis condition B: Retention time=1.31 min; ESI-MS(+) m/z [M+H]⁺: 1983.8.

### Preparation of Compound 2145

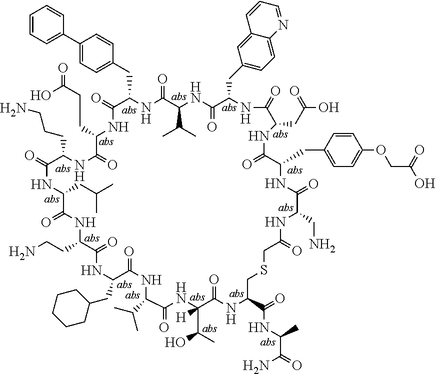 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc3ncccc3c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2145 was prepared on a 23 μmol scale. The yield of the product was 2.8 mg, and its estimated purity by LCMS analysis was 98.3%. Analysis condition A: Retention time=1.35 min; ESI-MS(+) m/z [M+2H]²⁺: 993.1.

### Preparation of Compound 2146

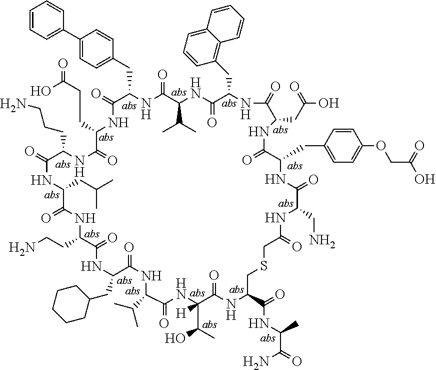 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccc3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2146 was prepared on a 23 μmol scale. The yield of the product was 6.7 mg, and its estimated purity by LCMS analysis was 85.6%. Analysis condition B: Retention time=1.66 min; ESI-MS(+) m/z [M+H]⁺: 1984.1.

### Preparation of Compound 2147

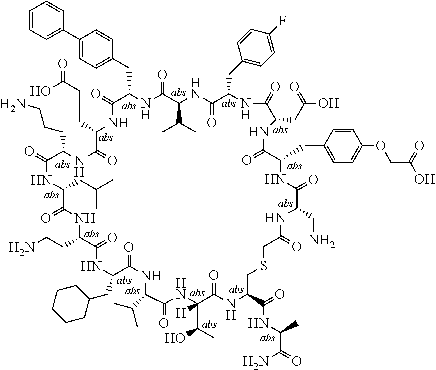 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc(F)cc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2147 was prepared on a 23 μmol scale. The yield of the product was 3.9 mg, and its estimated purity by LCMS analysis was 100%. Analysis condition A: Retention time=1.53 min; ESI-MS(+) m/z [M+H]⁺: 1951.

### Preparation of Compound 2148

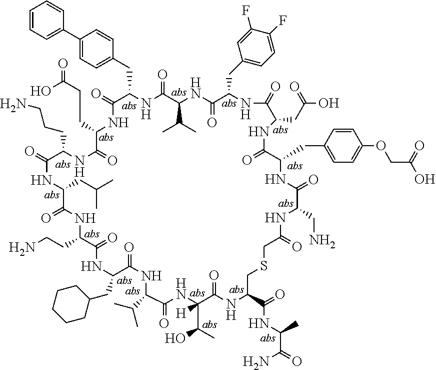 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc(F)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2148 was prepared on a 23 μmol scale. The yield of the product was 2.3 mg, and its estimated purity by LCMS analysis was 100%. Analysis condition A: Retention time=1.56 min; ESI-MS(+) m/z [M+H]⁺: 1968.8.

### Preparation of Compound 2149

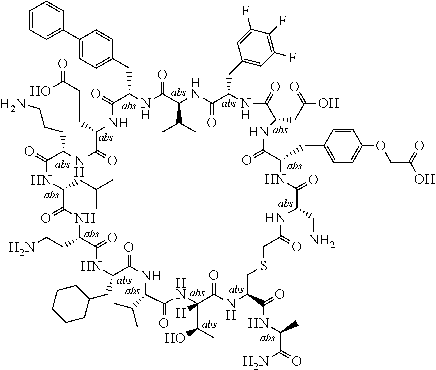 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cc(F)c(F)c(F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2149 was prepared on a 23 μmol scale. The yield of the product was 3.8 mg, and its estimated purity by LCMS analysis was 85.1%. Analysis condition B: Retention time=1.6 min; ESI-MS(+) m/z [M+H]⁺: 1987.

### Preparation of Compound 2150

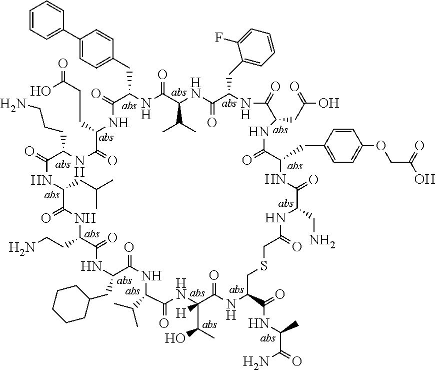 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2150 was prepared on a 23 μmol scale. The yield of the product was 2.1 mg, and its estimated purity by LCMS analysis was 94%. Analysis condition A: Retention time=1.49 min; ESI-MS(+) m/z [M+H]⁺: 1951.

### Preparation of Compound 2151

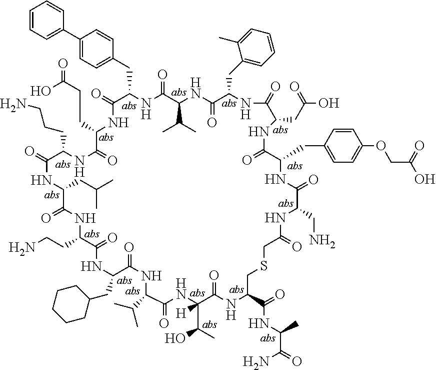 [SMILES: Cc1ccccc1C[C@@H]1NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCC(=O)O)NC(=O)[C@H](Cc2ccc(-c3ccccc3)cc2)NC(=O)[C@H](C(C)C)NC1=O]

Compound 2151 was prepared on a 23 μmol scale. The yield of the product was 3.3 mg, and its estimated purity by LCMS analysis was 84.2%. Analysis condition B: Retention time=1.48 min; ESI-MS(+) m/z [M+H]⁺: 1947.3.

### Preparation of Compound 2152

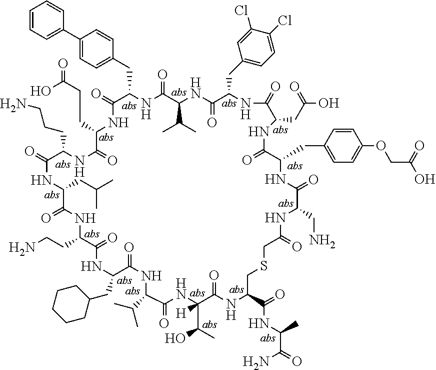 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccc(Cl)c(Cl)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2152 was prepared on a 23 μmol scale. The yield of the product was 2.3 mg, and its estimated purity by LCMS analysis was 98.9%. Analysis condition A: Retention time=1.5 min; ESI-MS(+) m/z [M+2H]²⁺: 1002.3.

### Preparation of Compound 2153

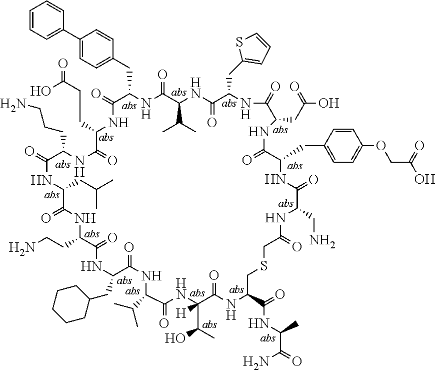 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2153 was prepared on a 23 μmol scale. The yield of the product was 2.6 mg, and its estimated purity by LCMS analysis was 87.7%. Analysis condition B: Retention time=1.39 min; ESI-MS(+) m/z [M+H]⁺: 1939.2.

### Preparation of Compound 2154

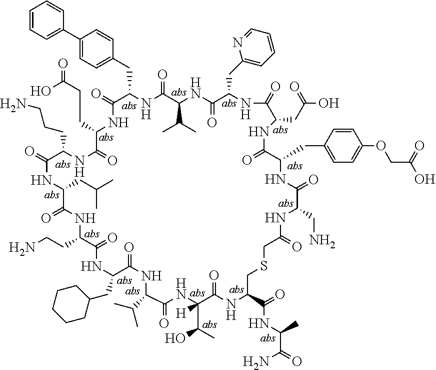 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccn2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2154 was prepared on a 23 μmol scale. The yield of the product was 6 mg, and its estimated purity by LCMS analysis was 94.6%. Analysis condition A: Retention time=1.47, 1.5 min; ESI-MS(+) m/z [M+H]⁺: 1935.

### Preparation of Compound 2155

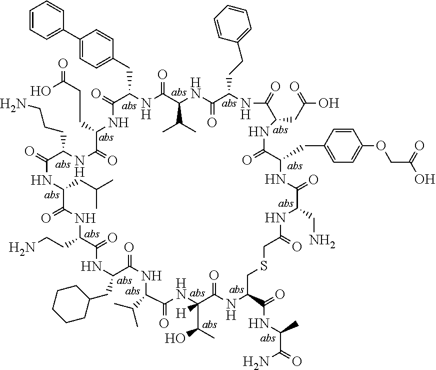 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](CCc2ccccc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2155 was prepared on a 23 μmol scale. The yield of the product was 5.3 mg, and its estimated purity by LCMS analysis was 85%. Analysis condition B: Retention time=1.5 min; ESI-MS(+) m/z [M+H]⁺: 1947.2.

### Preparation of Compound 2156

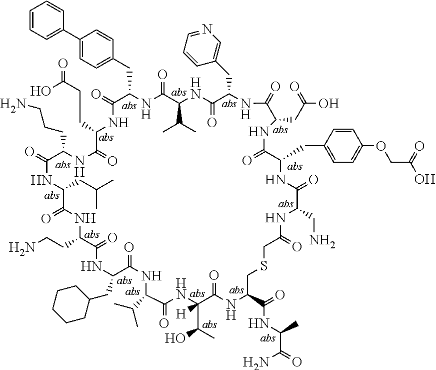 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccnc2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2156 was prepared on a 23 μmol scale. The yield of the product was 2 mg, and its estimated purity by LCMS analysis was 100%. Analysis condition A: Retention time=1.42 min; ESI-MS(+) m/z [M+H]⁺: 1934.3.

### Preparation of Compound 2157

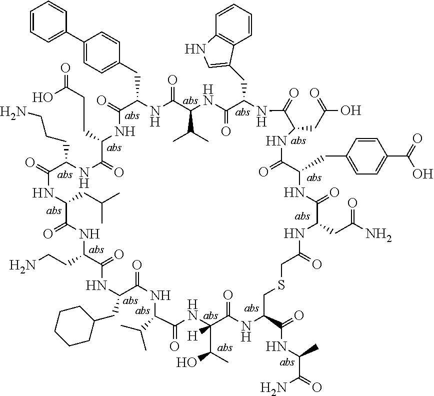 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2157 was prepared on a 23 μmol scale. The yield of the product was 3 mg, and its estimated purity by LCMS analysis was 85.7%. Analysis condition B: Retention time=1.51 min; ESI-MS(+) m/z [M+H]⁺: 1971.2.

### Preparation of Compound 2158

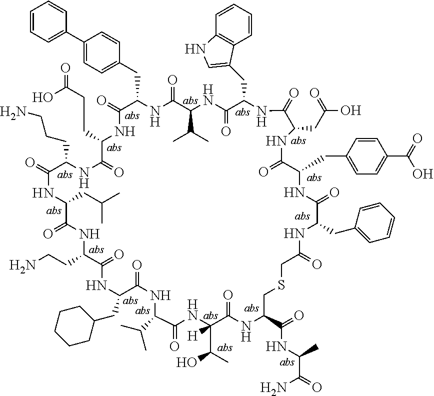 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2158 was prepared on a 23 μmol scale. The yield of the product was 4.8 mg, and its estimated purity by LCMS analysis was 85.9%. Analysis condition A: Retention time=1.48 min; ESI-MS(+) m/z [M+2H]²⁺: 1002.4.

### Preparation of Compound 2159

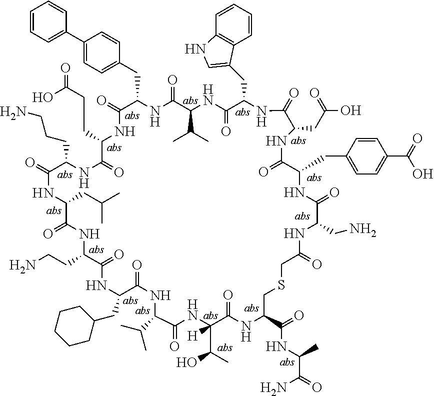 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2159 was prepared on a 23 μmol scale. The yield of the product was 3.3 mg, and its estimated purity by LCMS analysis was 97.3%. Analysis condition B: Retention time=1.52 min; ESI-MS(+) m/z [M+H]⁺: 1941.9.

### Preparation of Compound 2160

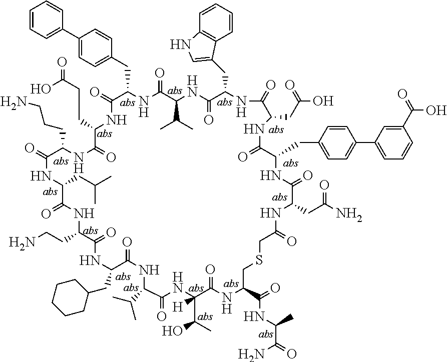 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2160 was prepared on a 23 μmol scale. The yield of the product was 3.5 mg, and its estimated purity by LCMS analysis was 94.9%. Analysis condition A: Retention time=1.55 min; ESI-MS(+) m/z [M+2H]²⁺: 1024.2.

### Preparation of Compound 2161

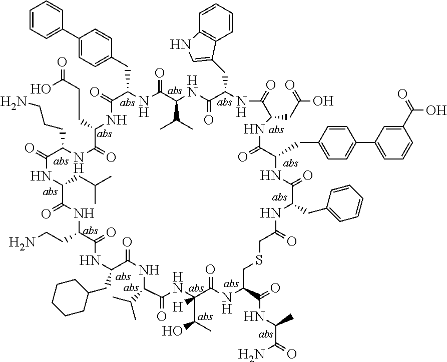 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2161 was prepared on a 23 μmol scale. The yield of the product was 2.2 mg, and its estimated purity by LCMS analysis was 91.8%. Analysis condition B: Retention time=1.87 min; ESI-MS(+) m/z [M+2H]²⁺: 1040.2.

### Preparation of Compound 2162

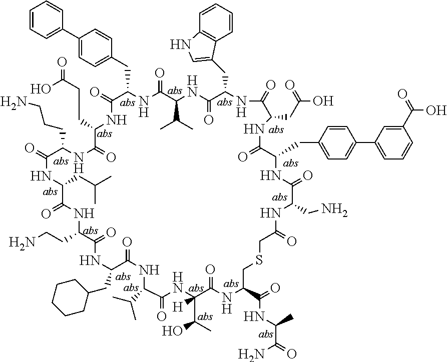 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2162 was prepared on a 23 μmol scale. The yield of the product was 2.4 mg, and its estimated purity by LCMS analysis was 97.4%. Analysis condition B: Retention time=1.52 min; ESI-MS(+) m/z [M+2H]²⁺: 1010.3.

### Preparation of Compound 2163

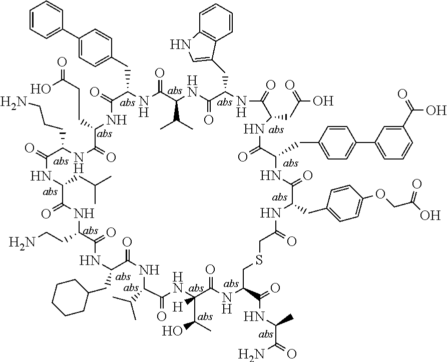 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2163 was prepared on a 23 μmol scale. The yield of the product was 8.7 mg, and its estimated purity by LCMS analysis was 85.6%. Analysis condition A: Retention time=1.44 min; ESI-MS(+) m/z [M+2H]²⁺: 1077.2.

### Preparation of Compound 2164

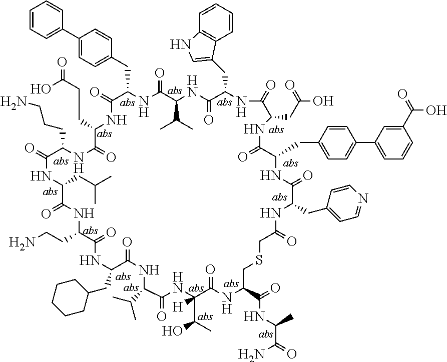 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2164 was prepared on a 23 μmol scale. The yield of the product was 3.1 mg, and its estimated purity by LCMS analysis was 97%. Analysis condition A: Retention time=1.51 min; ESI-MS(+) m/z [M+2H]²⁺: 1040.9.

### Preparation of Compound 2165

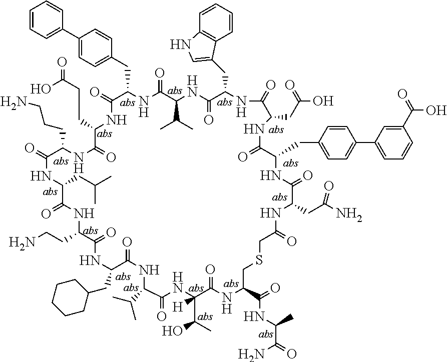 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2165 was prepared on a 23 μmol scale. The yield of the product was 3.9 mg, and its estimated purity by LCMS analysis was 90.6%. Analysis condition B: Retention time=1.72 min; ESI-MS(+) m/z [M+2H]²⁺: 1023.2.

### Preparation of Compound 2166

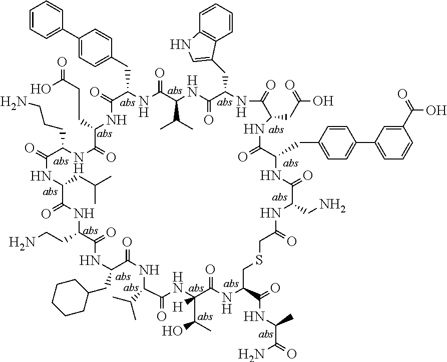 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2166 was prepared on a 23 μmol scale. The yield of the product was 7.6 mg, and its estimated purity by LCMS analysis was 95.7%. Analysis condition B: Retention time=1.62 min; ESI-MS(+) m/z [M+2H]²⁺: 1010.2.

### Preparation of Compound 2167

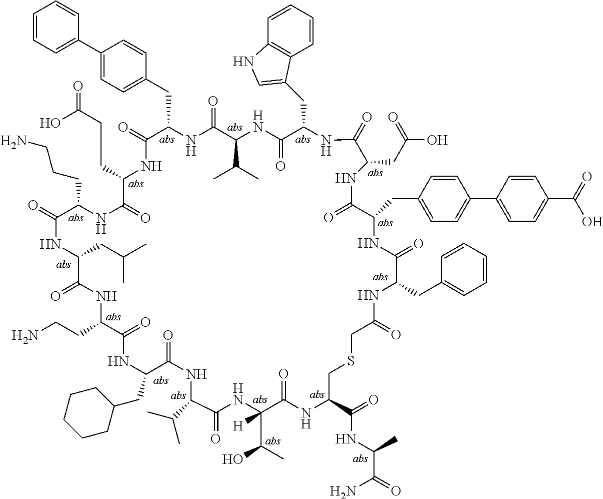 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3ccc(C(=O)O)cc3)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2167 was prepared on a 23 μmol scale. The yield of the product was 4.9 mg, and its estimated purity by LCMS analysis was 91.9%. Analysis condition A: Retention time=1.56 min; ESI-MS(+) m/z [M+2H]²⁺: 1040.1.

### Preparation of Compound 2168

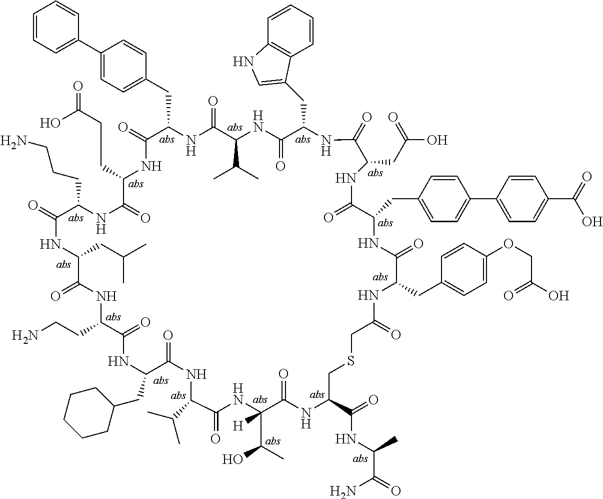 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3ccc(C(=O)O)cc3)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2168 was prepared on a 23 μmol scale. The yield of the product was 6.2 mg, and its estimated purity by LCMS analysis was 91.4%. Analysis condition B: Retention time=1.77 min; ESI-MS(+) m/z [M+2H]²⁺: 1077.1.

### Preparation of Compound 2169

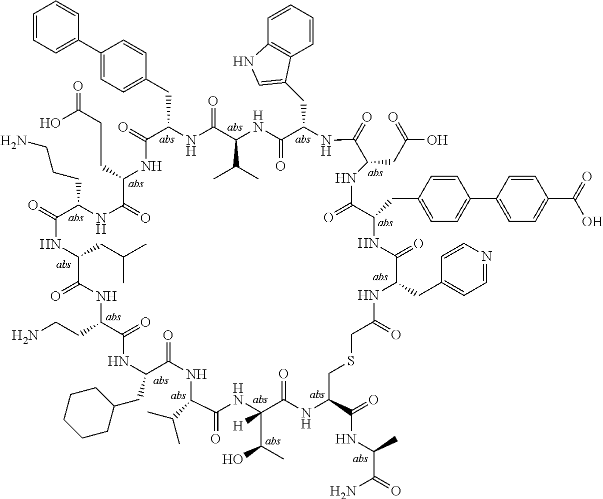 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3ccc(C(=O)O)cc3)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2169 was prepared on a 23 μmol scale. The yield of the product was 5.7 mg, and its estimated purity by LCMS analysis was 90.9%. Analysis condition B: Retention time=1.54 min; ESI-MS(+) m/z [M+2H]²⁺: 1041.1.

### Preparation of Compound 2170

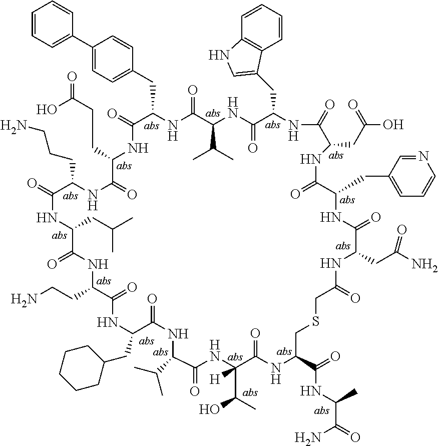 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2170 was prepared on a 23 μmol scale. The yield of the product was 4 mg, and its estimated purity by LCMS analysis was 92.4%. Analysis condition B: Retention time=1.55 min; ESI-MS(+) m/z [M+H]⁺: 1927.

### Preparation of Compound 2171

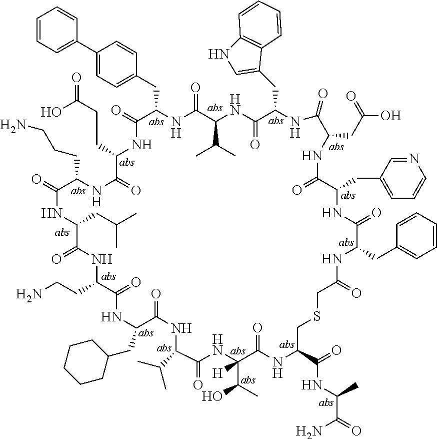 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2171 was prepared on a 23 μmol scale. The yield of the product was 4.4 mg, and its estimated purity by LCMS analysis was 100%. Analysis condition B: Retention time=1.61 min; ESI-MS(+) m/z [M+H]⁺: 1960.3.

### Preparation of Compound 2172

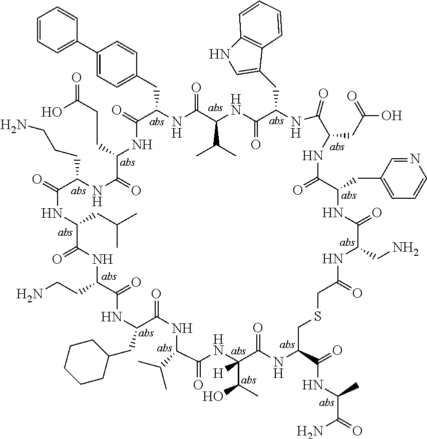 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2172 was prepared on a 23 μmol scale. The yield of the product was 4 mg, and its estimated purity by LCMS analysis was 91.5%. Analysis condition B: Retention time=1.5 min; ESI-MS(+) m/z [M+H]⁺: 1898.9.

### Preparation of Compound 2173

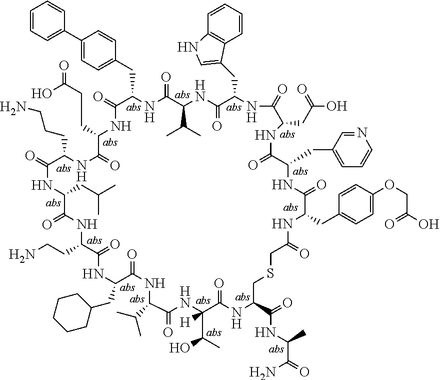 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2173 was prepared on a 23 μmol scale. The yield of the product was 4.3 mg, and its estimated purity by LCMS analysis was 90.3%. Analysis condition B: Retention time=1.58 min; ESI-MS(+) m/z [M+2H]²⁺: 1018.1.

### Preparation of Compound 2174

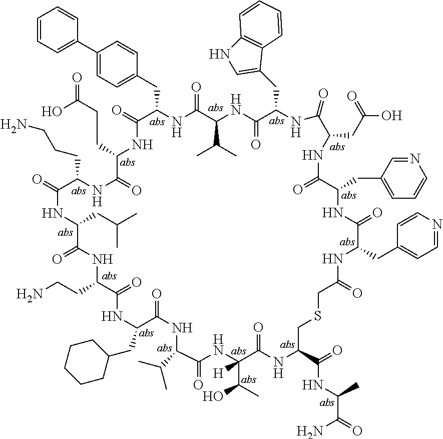 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2174 was prepared on a 23 μmol scale. The yield of the product was 3.1 mg, and its estimated purity by LCMS analysis was 91.3%. Analysis condition B: Retention time=1.49 min; ESI-MS(+) m/z [M+H]⁺: 1961.

### Preparation of Compound 2175

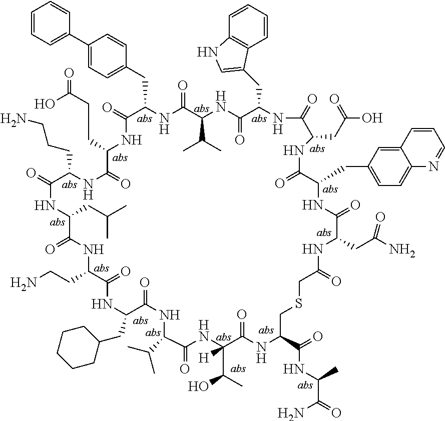 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2175 was prepared on a 21 μmol scale. The yield of the product was 3.5 mg, and its estimated purity by LCMS analysis was 90.3%. Analysis condition B: Retention time=1.49 min; ESI-MS(+) m/z [M+H]⁺: 1976.8.

### Preparation of Compound 2176

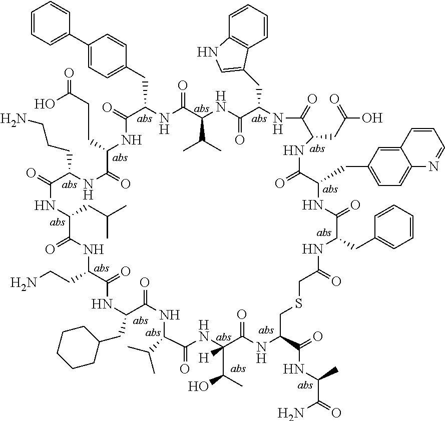 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2176 was prepared on a 21 μmol scale. The yield of the product was 1.6 mg, and its estimated purity by LCMS analysis was 97.5%. Analysis condition A: Retention time=1.72 min; ESI-MS(+) m/z [M+2H]²⁺: 1005.9.

### Preparation of Compound 2177

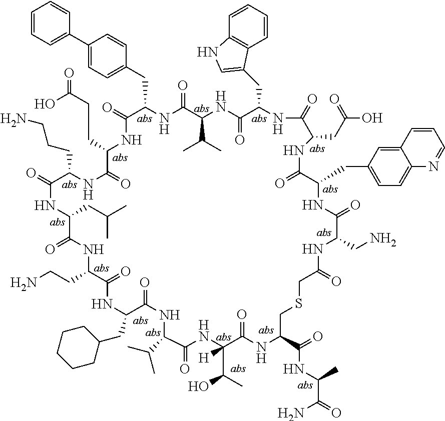 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2177 was prepared on a 21 μmol scale. The yield of the product was 3.6 mg, and its estimated purity by LCMS analysis was 91.6%. Analysis condition B: Retention time=1.52 min; ESI-MS(+) m/z [M+H]⁺: 1949.2.

### Preparation of Compound 2178

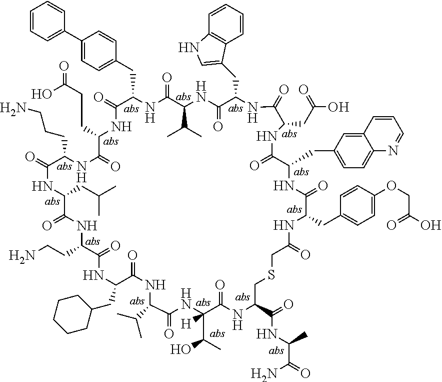 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2178 was prepared on a 21 μmol scale. The yield of the product was 3.6 mg, and its estimated purity by LCMS analysis was 97%. Analysis condition B: Retention time=1.6 min; ESI-MS(+) m/z [M+2H]²⁺: 1043.1.

### Preparation of Compound 2179

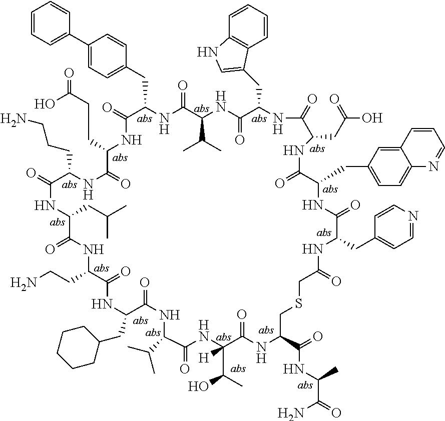 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc3ncccc3c2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2179 was prepared on a 21 μmol scale. The yield of the product was 2.6 mg, and its estimated purity by LCMS analysis was 92.5%. Analysis condition A: Retention time=1.58 min; ESI-MS(+) m/z [M+2H]²⁺: 1006.2.

### Preparation of Compound 2180

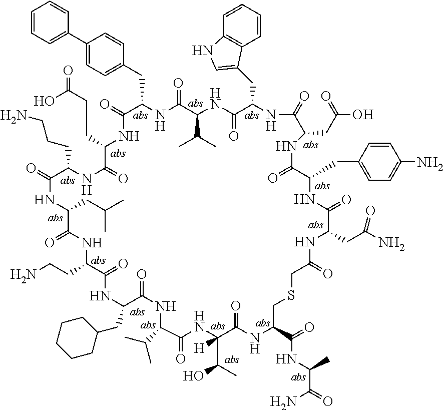 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2180 was prepared on a 21 μmol scale. The yield of the product was 3 mg, and its estimated purity by LCMS analysis was 90%. Analysis condition B: Retention time=1.55 min; ESI-MS(+) m/z [M+2H]²⁺: 1941.

### Preparation of Compound 2181

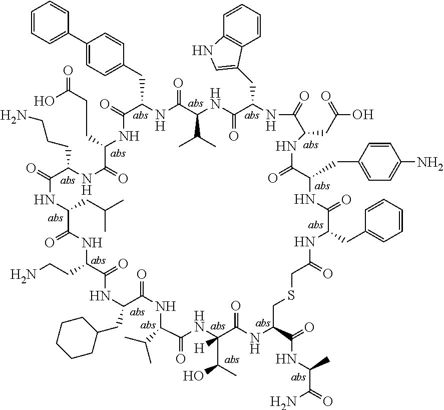 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2181 was prepared on a 21 μmol scale. The yield of the product was 2.3 mg, and its estimated purity by LCMS analysis was 91.7%. Analysis condition B: Retention time=1.63 min; ESI-MS(+) m/z [M+2H]²⁺: 987.3.

### Preparation of Compound 2182

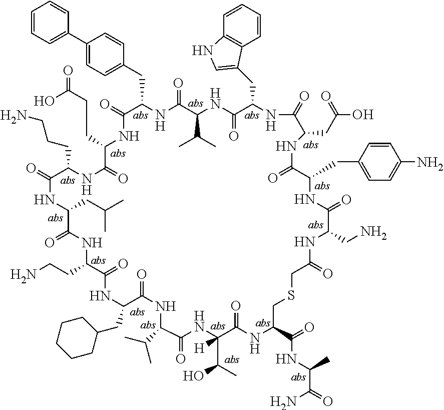 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2182 was prepared on a 21 μmol scale. The yield of the product was 1.8 mg, and its estimated purity by LCMS analysis was 84.6%. Analysis condition B: Retention time=1.48 min; ESI-MS(+) m/z [M+2H]²⁺: 957.7.

### Preparation of Compound 2183

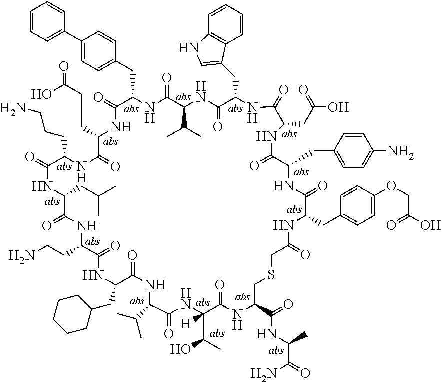 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2183 was prepared on a 21 μmol scale. The yield of the product was 2.2 mg, and its estimated purity by LCMS analysis was 93.9%. Analysis condition B: Retention time=1.57 min; ESI-MS(+) m/z [M+2H]²⁺: 1025.1.

### Preparation of Compound 2184

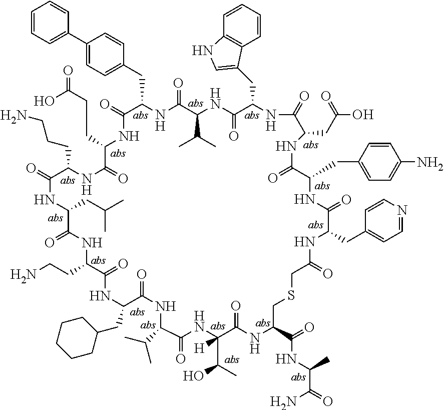 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(N)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2184 was prepared on a 21 μmol scale. The yield of the product was 1.8 mg, and its estimated purity by LCMS analysis was 87.2%. Analysis condition B: Retention time=1.47 min; ESI-MS(+) m/z [M+2H]²⁺: 988.2.

### Preparation of Compound 2185

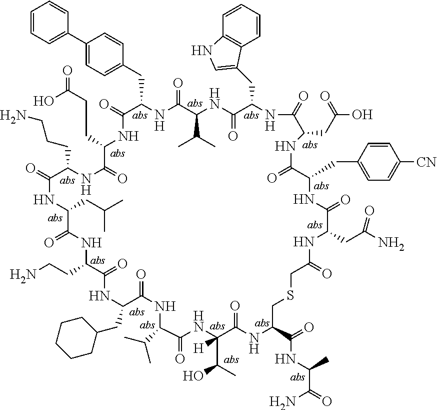 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2185 was prepared on a 21 μmol scale. The yield of the product was 2.8 mg, and its estimated purity by LCMS analysis was 93.2%. Analysis condition A: Retention time=1.63 min; ESI-MS(+) m/z [M+2H]²⁺: 977.1.

### Preparation of Compound 2186

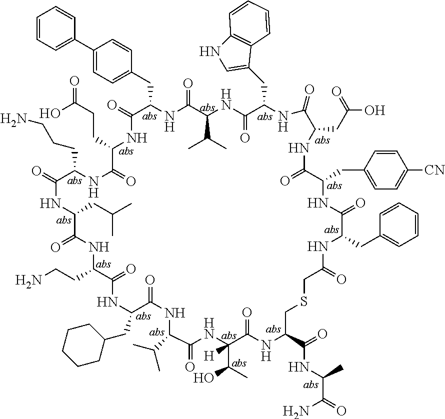 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2186 was prepared on a 21 μmol scale. The yield of the product was 8.4 mg, and its estimated purity by LCMS analysis was 97.7%. Analysis condition B: Retention time=1.74 min; ESI-MS(+) m/z [M+2H]²⁺: 993.1.

### Preparation of Compound 2187

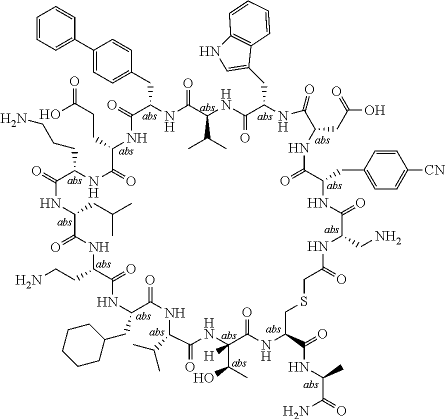 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2187 was prepared on a 21 μmol scale. The yield of the product was 4 mg, and its estimated purity by LCMS analysis was 90.1%. Analysis condition B: Retention time=1.59 min; ESI-MS(+) m/z [M+2H]²⁺: 962.1.

### Preparation of Compound 2188

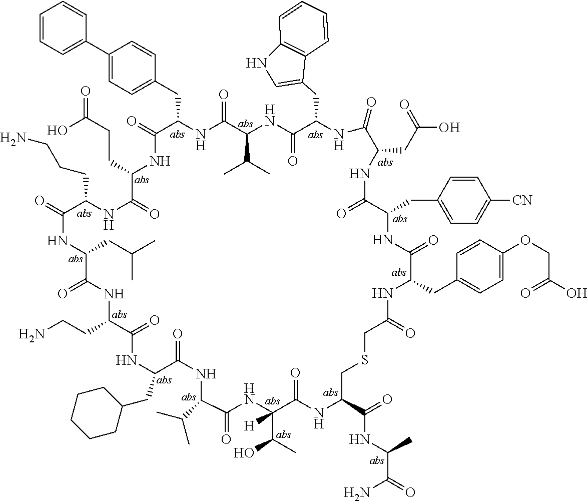 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2188 was prepared on a 21 μmol scale. The yield of the product was 3.5 mg, and its estimated purity by LCMS analysis was 100%. Analysis condition A: Retention time=1.63 min; ESI-MS(+) m/z [M+2H]²⁺: 1030.

### Preparation of Compound 2189

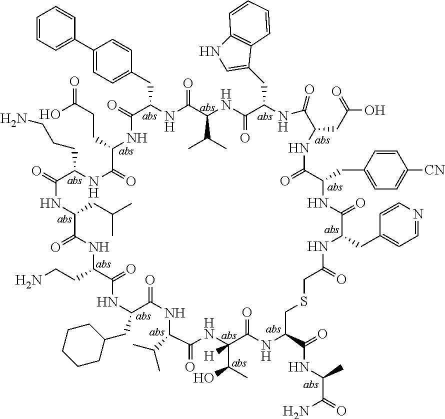 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2189 was prepared on a 21 μmol scale. The yield of the product was 3.3 mg, and its estimated purity by LCMS analysis was 90.6%. Analysis condition A: Retention time=1.56 min; ESI-MS(+) m/z [M+3H]³⁺: 662.3.

### Preparation of Compound 2190

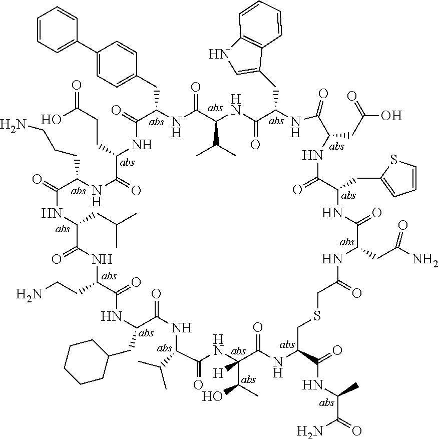 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2190 was prepared on a 21 μmol scale. The yield of the product was 6.8 mg, and its estimated purity by LCMS analysis was 96.6%. Analysis condition B: Retention time=1.64 min; ESI-MS(+) m/z [M+H]⁺: 1932.

### Preparation of Compound 2191

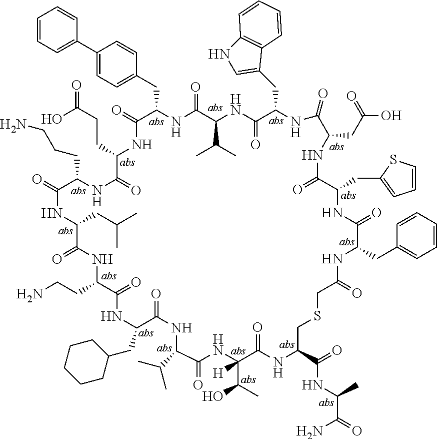 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2191 was prepared on a 21 μmol scale. The yield of the product was 2.2 mg, and its estimated purity by LCMS analysis was 92.7%. Analysis condition B: Retention time=1.82 min; ESI-MS(+) m/z [M+H]⁺: 1965.1.

### Preparation of Compound 2192

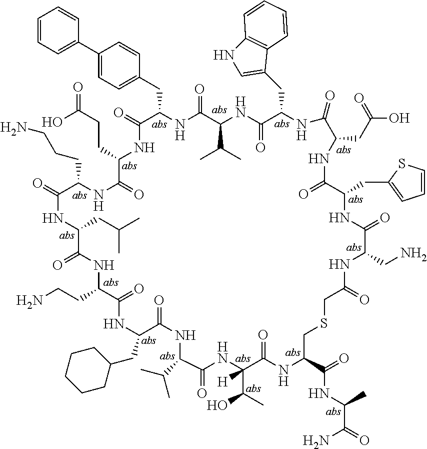 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2192 was prepared on a 21 μmol scale. The yield of the product was 3.7 mg, and its estimated purity by LCMS analysis was 93%. Analysis condition A: Retention time=1.6 min; ESI-MS(+) m/z [M+2H]²⁺: 953.1.

### Preparation of Compound 2193

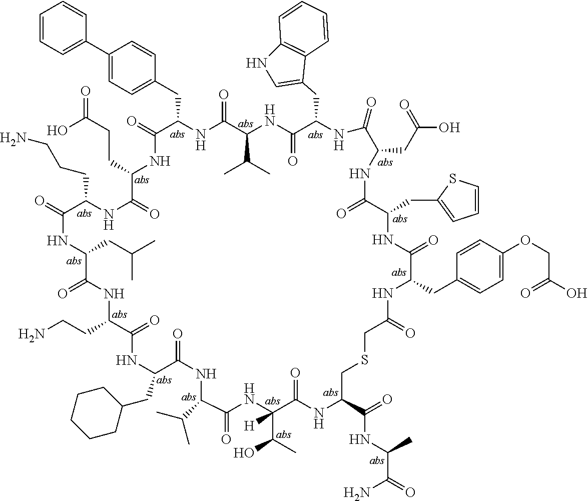 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2193 was prepared on a 21 μmol scale. The yield of the product was 5.5 mg, and its estimated purity by LCMS analysis was 100%. Analysis condition A: Retention time=1.65 min; ESI-MS(+) m/z [M+2H]²⁺: 1020.3.

### Preparation of Compound 2194

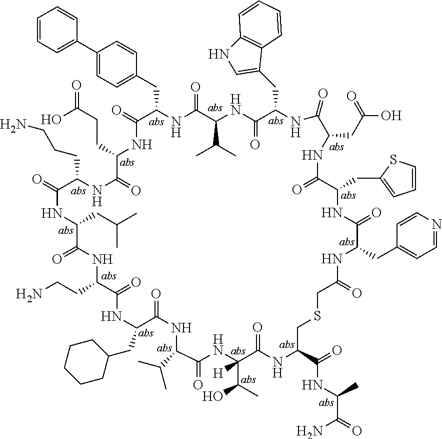 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccs2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2194 was prepared on a 21 μmol scale. The yield of the product was 3.5 mg, and its estimated purity by LCMS analysis was 100%. Analysis condition A: Retention time=1.7 min; ESI-MS(+) m/z [M+H]⁺: 1965.9.

### Preparation of Compound 2195

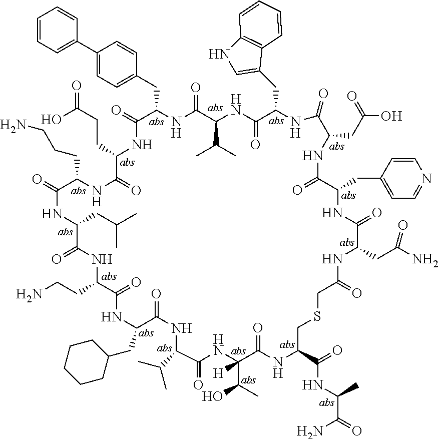 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2195 was prepared on a 21 μmol scale. The yield of the product was 6.7 mg, and its estimated purity by LCMS analysis was 94.6%. Analysis condition B: Retention time=1.5 min; ESI-MS(+) m/z [M+2H]²⁺: 964.2.

### Preparation of Compound 2196

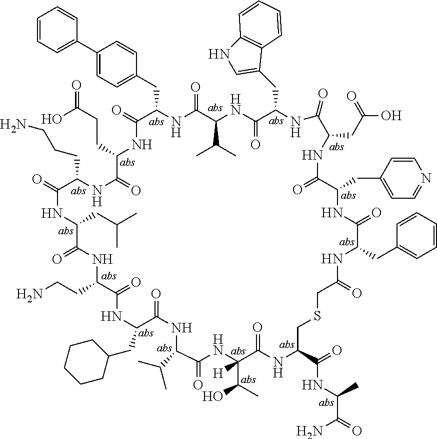 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2196 was prepared on a 21 μmol scale. The yield of the product was 6.3 mg, and its estimated purity by LCMS analysis was 92.7%. Analysis condition A: Retention time=1.71 min; ESI-MS(+) m/z [M+2H]²⁺: 981.

### Preparation of Compound 2197

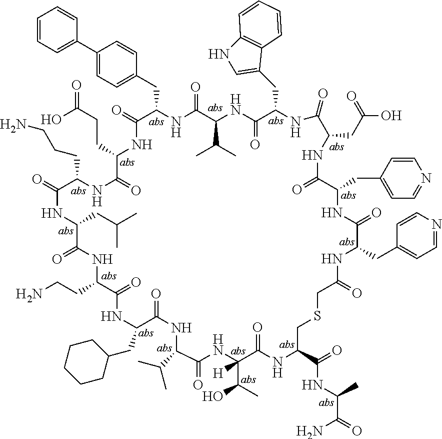 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2197 was prepared on a 21 μmol scale. The yield of the product was 2.8 mg, and its estimated purity by LCMS analysis was 100%. Analysis condition A: Retention time=1.6 min; ESI-MS(+) m/z [M+2H]²⁺: 981.1.

### Preparation of Compound 2198

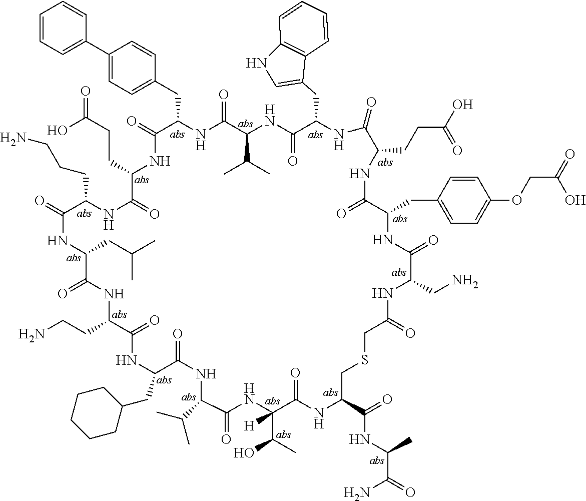 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CCC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2198 was prepared on a 21 μmol scale. The yield of the product was 2.9 mg, and its estimated purity by LCMS analysis was 95.2%. Analysis condition A: Retention time=1.58 min; ESI-MS(+) m/z [M+2H]²⁺: 994.1.

### Preparation of Compound 2199

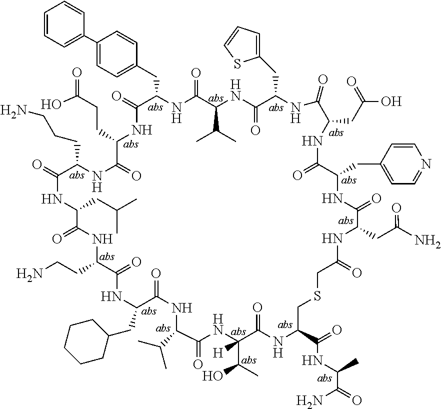 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccncc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2199 was prepared on a 23 μmol scale. The yield of the product was 1.7 mg, and its estimated purity by LCMS analysis was 100%. Analysis condition A: Retention time=1.57 min; ESI-MS(+) m/z [M+H]⁺: 1894.2.

### Preparation of Compound 2200

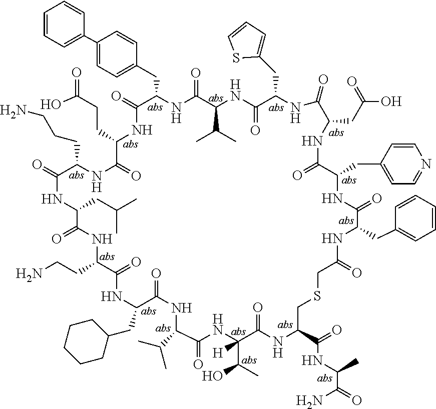 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccncc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2200 was prepared on a 23 μmol scale. The yield of the product was 3.1 mg, and its estimated purity by LCMS analysis was 91%. Analysis condition A: Retention time=1.65 min; ESI-MS(+) m/z [M+H]⁺: 1927.

### Preparation of Compound 2201

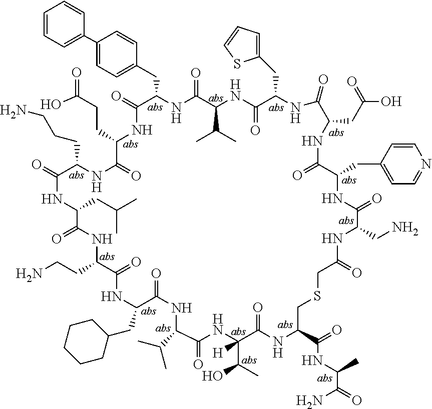 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2201 was prepared on a 23 μmol scale. The yield of the product was 2.9 mg, and its estimated purity by LCMS analysis was 90.7%. Analysis condition B: Retention time=1.42, 1.46 min; ESI-MS(+) m/z [M+2H]²⁺: 933.16, 933.16.

### Preparation of Compound 2202

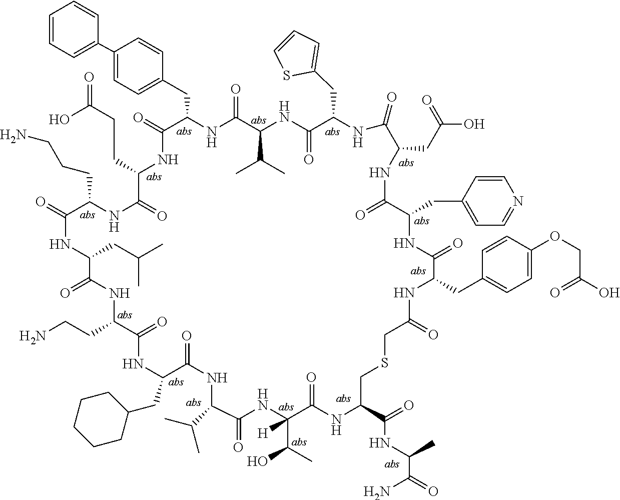 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2202 was prepared on a 23 μmol scale. The yield of the product was 4.5 mg, and its estimated purity by LCMS analysis was 91.7%. Analysis condition A: Retention time=1.45 min; ESI-MS(+) m/z [M+3H]³⁺: 668.

### Preparation of Compound 2203

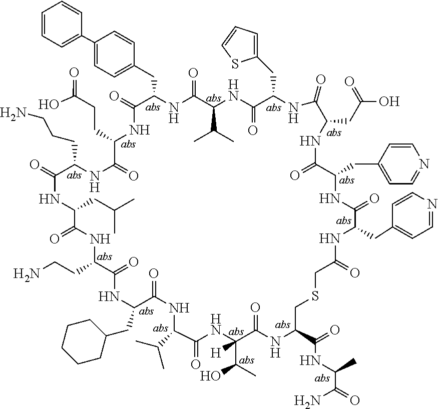 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2ccncc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2203 was prepared on a 23 μmol scale. The yield of the product was 1 mg, and its estimated purity by LCMS analysis was 93.6%. Analysis condition B: Retention time=1.38 min; ESI-MS(+) m/z [M+H]⁺: 1927.9.

### Preparation of Compound 2204

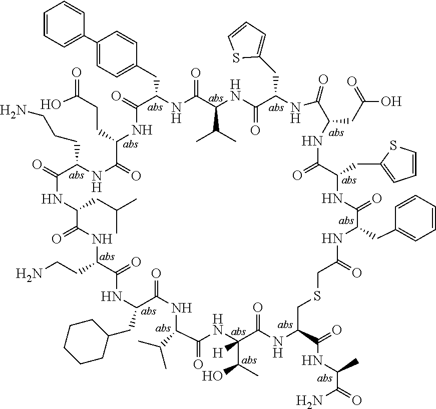 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2cccs2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2204 was prepared on a 23 μmol scale. The yield of the product was 4.1 mg, and its estimated purity by LCMS analysis was 100%. Analysis condition A: Retention time=1.8 min; ESI-MS(+) m/z [M+H]⁺: 1931.7.

### Preparation of Compound 2205

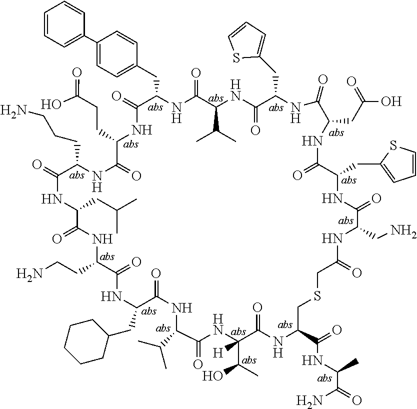 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2cccs2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2205 was prepared on a 23 μmol scale. The yield of the product was 2.3 mg, and its estimated purity by LCMS analysis was 91.3%. Analysis condition B: Retention time=1.61 min; ESI-MS(+) m/z [M+2H]²⁺: 936.1.

### Preparation of Compound 2206

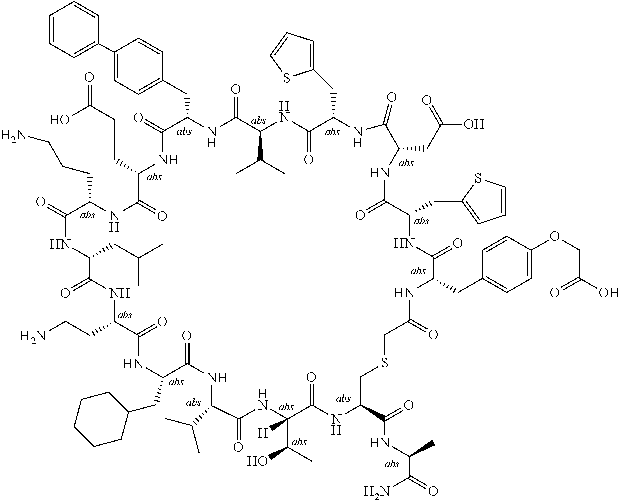 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2cccs2)NC(=O)[C@H](CC(=O)O)NC(=O)[C@H](Cc2cccs2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2206 was prepared on a 23 μmol scale. The yield of the product was 7.2 mg, and its estimated purity by LCMS analysis was 90.4%. Analysis condition B: Retention time=1.78 min; ESI-MS(+) m/z [M+2H]²⁺: 1004.1.

### Preparation of Compound 2207

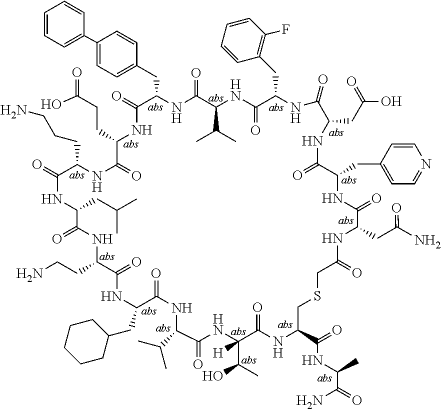 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccncc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2207 was prepared on a 23 μmol scale. The yield of the product was 2.5 mg, and its estimated purity by LCMS analysis was 100%. Analysis condition B: Retention time=1.54 min; ESI-MS(+) m/z [M+2H]²⁺: 954.2.

### Preparation of Compound 2208

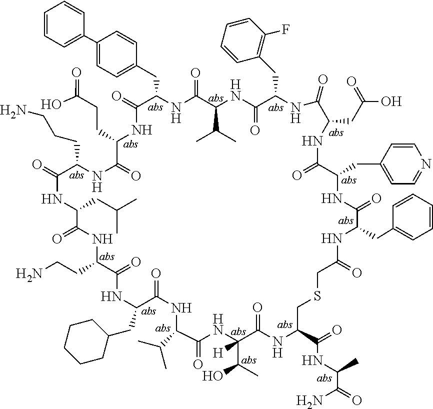 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccncc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2208 was prepared on a 23 μmol scale. The yield of the product was 2.2 mg, and its estimated purity by LCMS analysis was 92.3%. Analysis condition A: Retention time=1.56 min; ESI-MS(+) m/z [M+2H]²⁺: 970.3.

### Preparation of Compound 2209

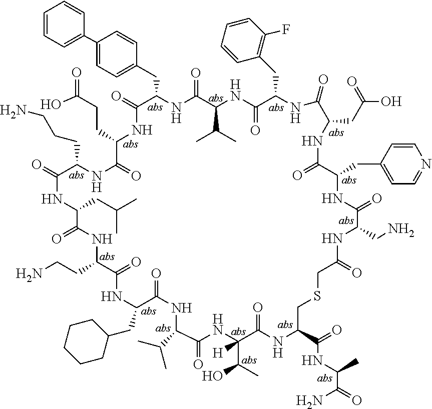 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccncc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2209 was prepared on a 23 μmol scale. The yield of the product was 1.8 mg, and its estimated purity by LCMS analysis was 100%. Analysis condition A: Retention time=1.44 min; ESI-MS(+) m/z [M+2H]²⁺: 940.2.

### Preparation of Compound 2210

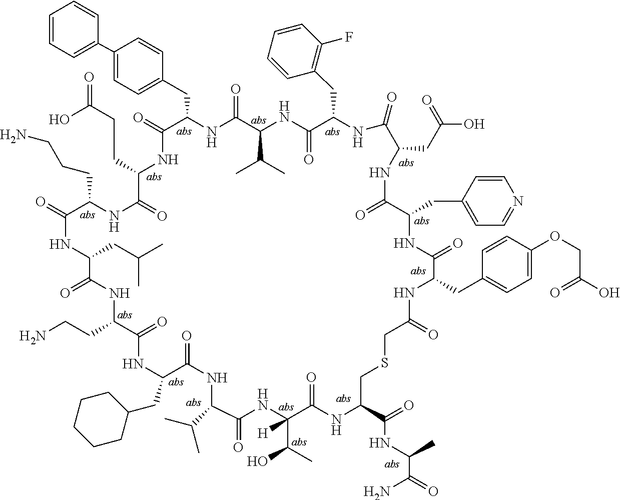 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccncc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2210 was prepared on a 23 μmol scale. The yield of the product was 2.8 mg, and its estimated purity by LCMS analysis was 100%. Analysis condition B: Retention time=1.45 min; ESI-MS(+) m/z [M+2H]²⁺: 1007.3.

### Preparation of Compound 2211

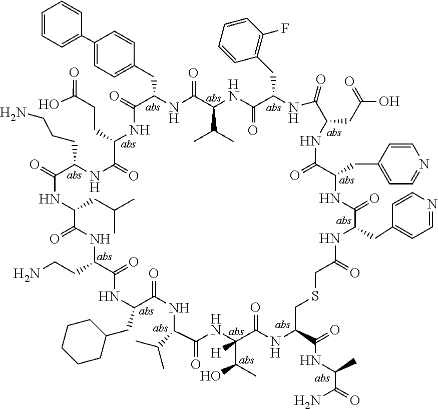 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccncc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2211 was prepared on a 23 μmol scale. The yield of the product was 2.1 mg, and its estimated purity by LCMS analysis was 97.7%. Analysis condition A: Retention time=1.41 min; ESI-MS(+) m/z [M+2H]²⁺: 970.9.

### Preparation of Compound 2212

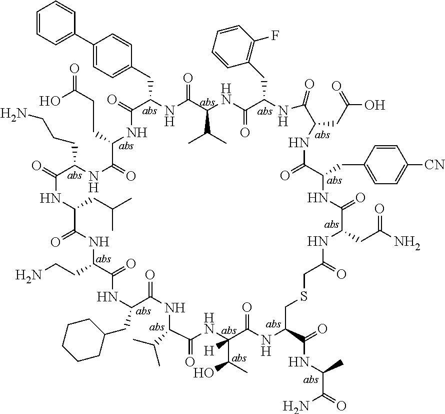 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(C#N)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2212 was prepared on a 23 μmol scale. The yield of the product was 4.4 mg, and its estimated purity by LCMS analysis was 96.3%. Analysis condition A: Retention time=1.53 min; ESI-MS(+) m/z [M+2H]²⁺: 965.9.

### Preparation of Compound 2213

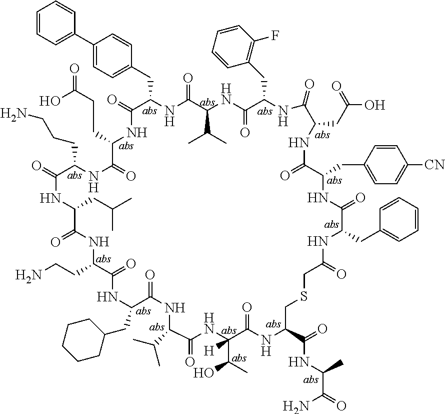 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(C#N)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2213 was prepared on a 23 μmol scale. The yield of the product was 3.7 mg, and its estimated purity by LCMS analysis was 93.7%. Analysis condition A: Retention time=1.64 min; ESI-MS(+) m/z [M+2H]²⁺: 982.3.

### Preparation of Compound 2214

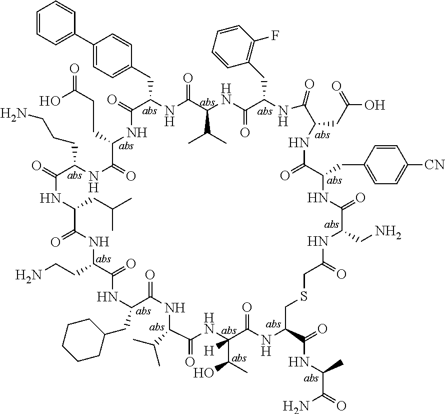 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(C#N)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2214 was prepared on a 23 μmol scale. The yield of the product was 4.6 mg, and its estimated purity by LCMS analysis was 92.2%. Analysis condition B: Retention time=1.63 min; ESI-MS(+) m/z [M+2H]²⁺: 951.3.

### Preparation of Compound 2215

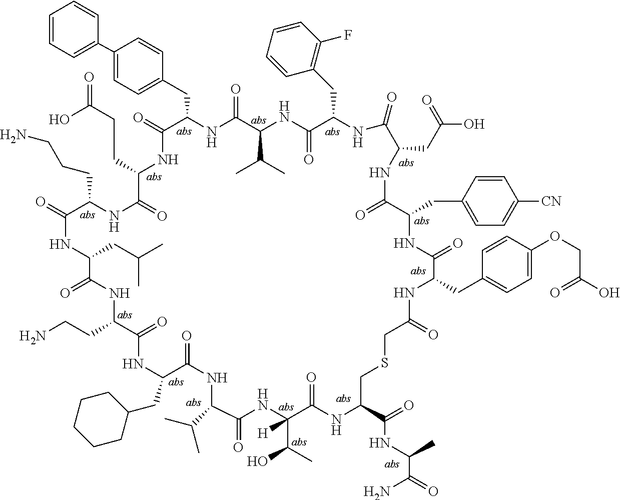 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(C#N)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2215 was prepared on a 23 μmol scale. The yield of the product was 6 mg, and its estimated purity by LCMS analysis was 95.3%. Analysis condition A: Retention time=1.45, 1.49 min; ESI-MS(+) m/z [M+2H]²⁺: 1020.

### Preparation of Compound 2216

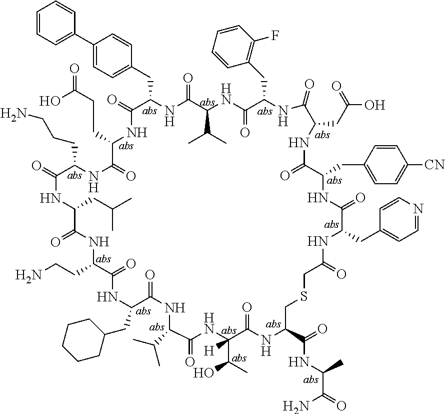 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2ccccc2F)NC(=O)[C@H](CC(=O)O)NC(=O)[C@H](Cc2ccc(C#N)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2216 was prepared on a 23 μmol scale. The yield of the product was 4.6 mg, and its estimated purity by LCMS analysis was 86%. Analysis condition B: Retention time=1.66 min; ESI-MS(+) m/z [M+H]⁺: 1964.9.

### Preparation of Compound 2217

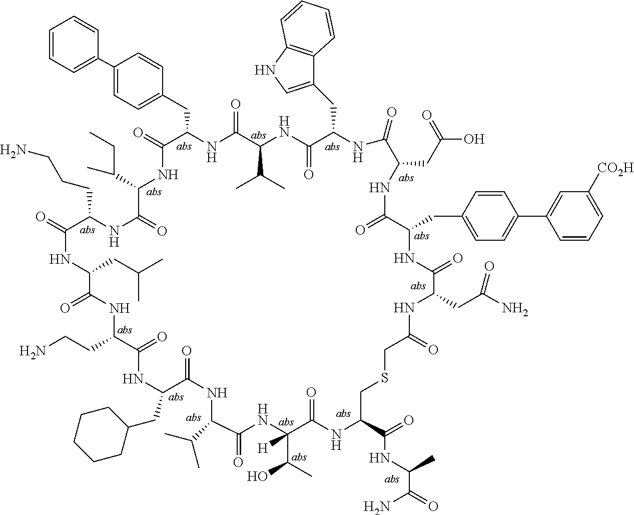 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2217 was prepared on a 23 μmol scale. The yield of the product was 5.4 mg, and its estimated purity by LCMS analysis was 92.9%. Analysis condition A: Retention time=1.82 min; ESI-MS(+) m/z [M+2H]²⁺: 1016.

### Preparation of Compound 2218

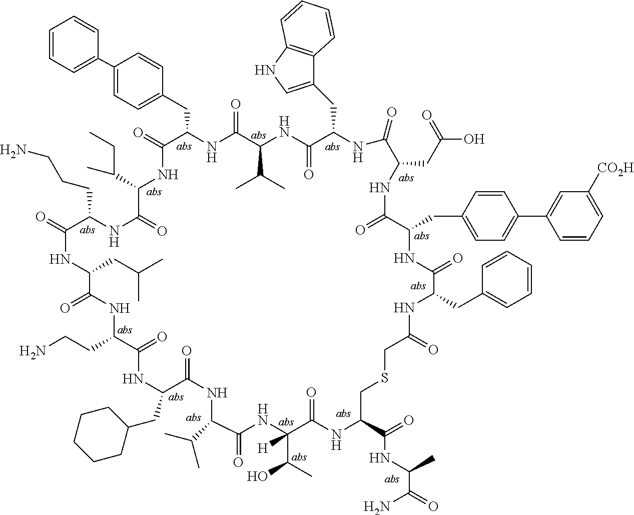 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2218 was prepared on a 23 μmol scale. The yield of the product was 6.9 mg, and its estimated purity by LCMS analysis was 85.3%. Analysis condition A: Retention time=1.86 min; ESI-MS(+) m/z [M+2H]²⁺: 1032.4.

### Preparation of Compound 2219

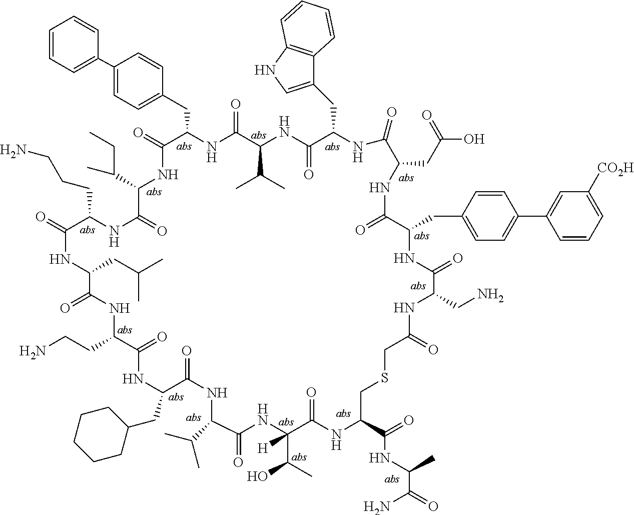 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2219 was prepared on a 23 μmol scale. The yield of the product was 4.7 mg, and its estimated purity by LCMS analysis was 97.8%. Analysis condition A: Retention time=1.77 min; ESI-MS(+) m/z [M+2H]²⁺: 1002.

### Preparation of Compound 2220

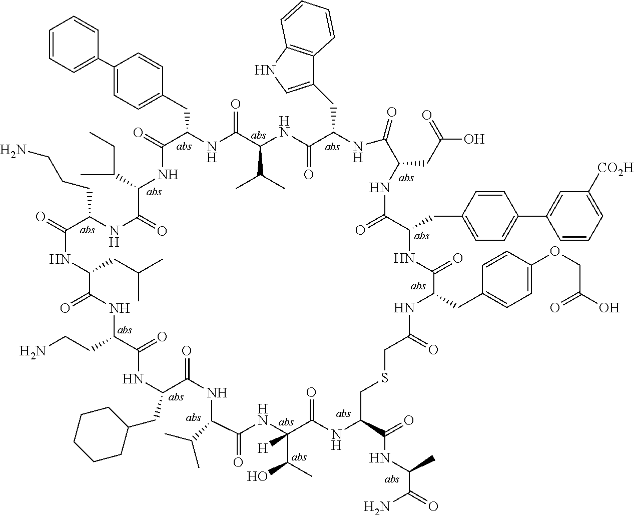 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2220 was prepared on a 23 μmol scale. The yield of the product was 4.5 mg, and its estimated purity by LCMS analysis was 100%. Analysis condition A: Retention time=1.95 min; ESI-MS(+) m/z [M+2H]²⁺: 1069.2.

### Preparation of Compound 2221

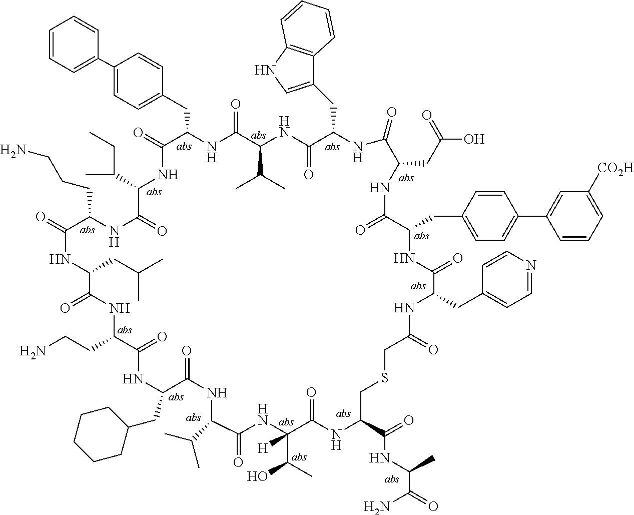 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2221 was prepared on a 23 μmol scale. The yield of the product was 3.1 mg, and its estimated purity by LCMS analysis was 91.7%. Analysis condition A: Retention time=1.84 min; ESI-MS(+) m/z [M+2H]²⁺: 1033.

### Preparation of Compound 2222

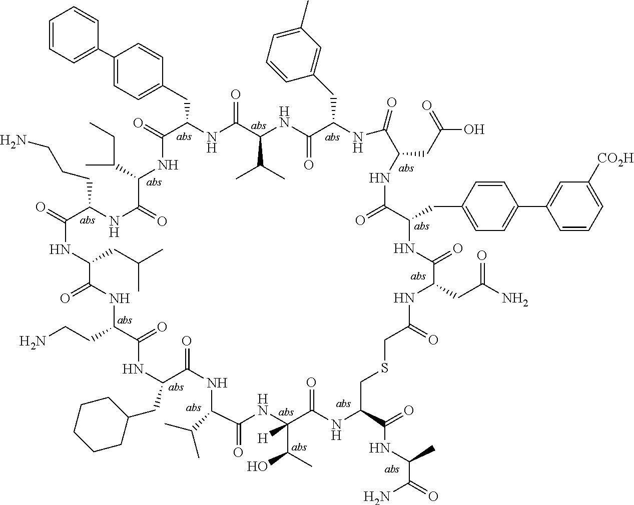 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(-c3cccc(C(=O)O)c3)cc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2222 was prepared on a 23 μmol scale. The yield of the product was 3.9 mg, and its estimated purity by LCMS analysis was 94.7%. Analysis condition B: Retention time=1.93 min; ESI-MS(+) m/z [M+2H]²⁺: 1003.2.

### Preparation of Compound 2223

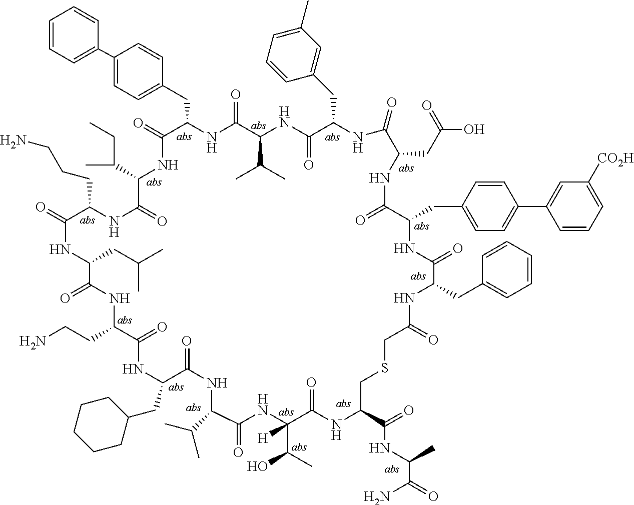 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(-c3cccc(C(=O)O)c3)cc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2223 was prepared on a 23 μmol scale. The yield of the product was 4.7 mg, and its estimated purity by LCMS analysis was 94.7%. Analysis condition A: Retention time=2.03 min; ESI-MS(+) m/z [M+2H]²⁺: 1020.

### Preparation of Compound 2224

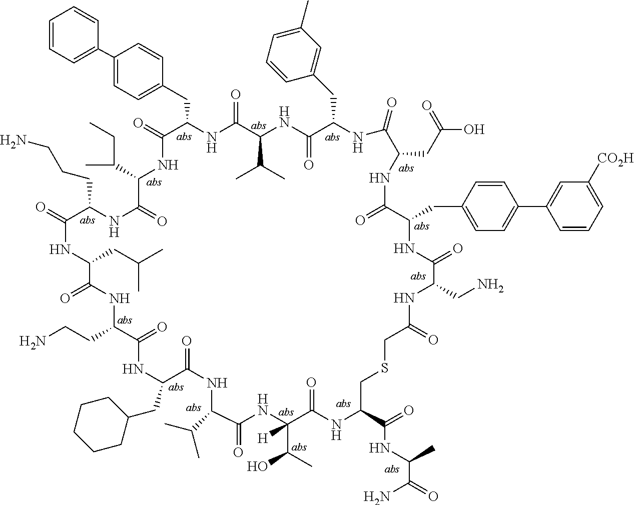 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(-c3cccc(C(=O)O)c3)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2224 was prepared on a 23 μmol scale. The yield of the product was 1.7 mg, and its estimated purity by LCMS analysis was 100%. Analysis condition B: Retention time=1.48 min; ESI-MS(+) m/z [M+2H]²⁺: 990.1.

### Preparation of Compound 2225

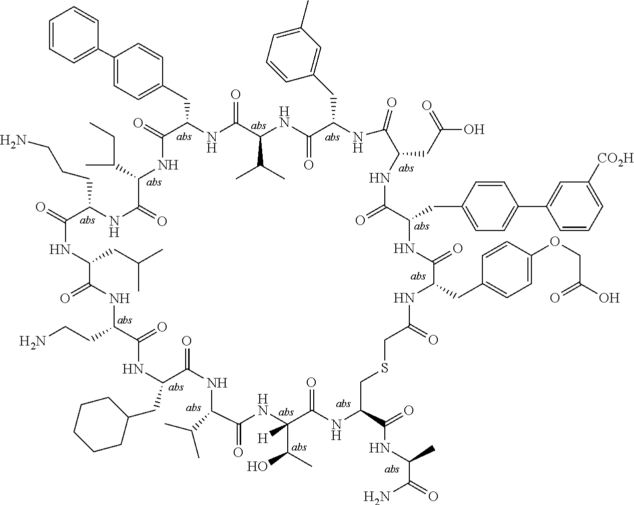 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(-c3cccc(C(=O)O)c3)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2225 was prepared on a 23 μmol scale. The yield of the product was 8.8 mg, and its estimated purity by LCMS analysis was 100%. Analysis condition A: Retention time=2.16 min; ESI-MS(+) m/z [M+2H]²⁺: 1057.2.

### Preparation of Compound 2226

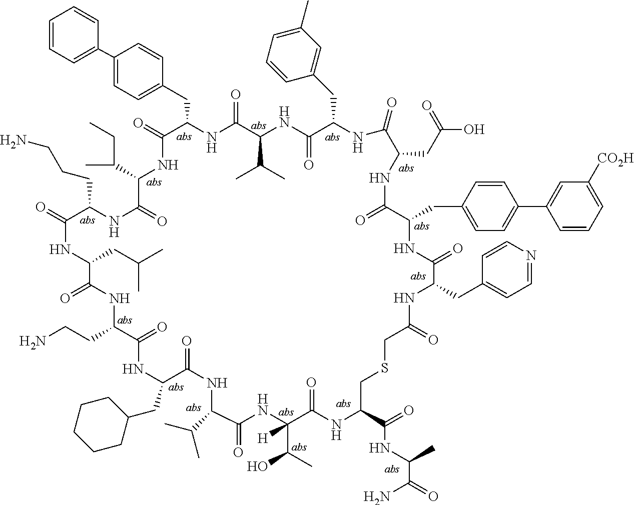 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(-c3cccc(C(=O)O)c3)cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2226 was prepared on a 23 μmol scale. The yield of the product was 2.1 mg, and its estimated purity by LCMS analysis was 98.3%. Analysis condition A: Retention time=1.88 min; ESI-MS(+) m/z [M+3H]³⁺: 680.8.

### Preparation of Compound 2227

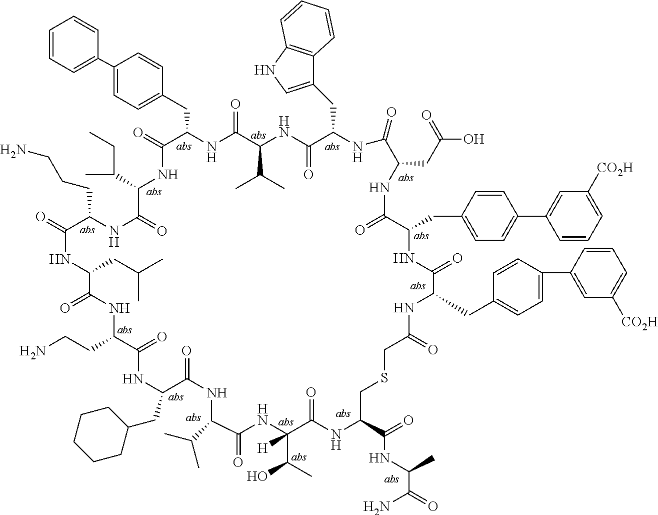 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(-c3cccc(C(=O)O)c3)cc2)NC(=O)[C@H](Cc2ccc(-c3cccc(C(=O)O)c3)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2227 was prepared on a 23 μmol scale. The yield of the product was 5.2 mg, and its estimated purity by LCMS analysis was 92.9%. Analysis condition A: Retention time=1.83 min; ESI-MS(+) m/z [M+2H]²⁺: 1090.1.

### Preparation of Compound 2228

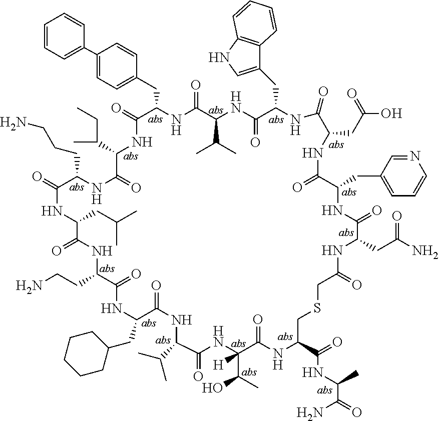 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2228 was prepared on a 23 μmol scale. The yield of the product was 4 mg, and its estimated purity by LCMS analysis was 97.5%. Analysis condition B: Retention time=1.48 min; ESI-MS(+) m/z [M+H]⁺: 1911.1.

### Preparation of Compound 2229

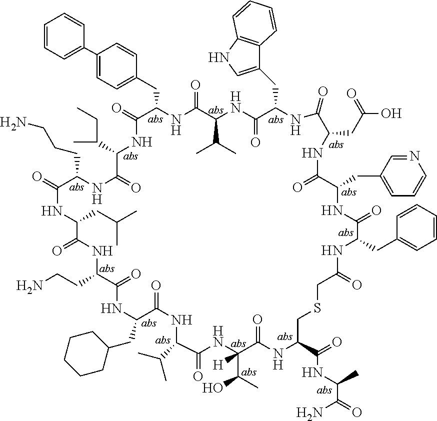 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2229 was prepared on a 23 μmol scale. The yield of the product was 4.3 mg, and its estimated purity by LCMS analysis was 87.8%. Analysis condition B: Retention time=1.57 min; ESI-MS(+) m/z [M+H]⁺: 1944.

### Preparation of Compound 2230

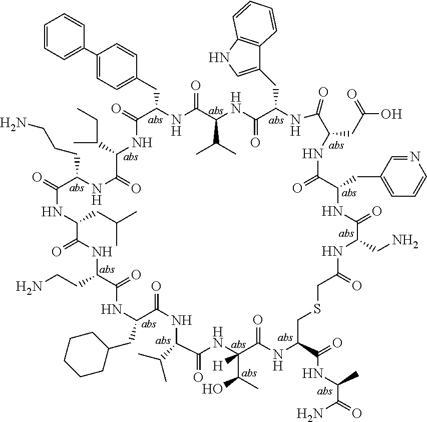 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2230 was prepared on a 23 μmol scale. The yield of the product was 3.8 mg, and its estimated purity by LCMS analysis was 91.1%. Analysis condition B: Retention time=1.42 min; ESI-MS(+) m/z [M+H]⁺: 1883.

### Preparation of Compound 2231

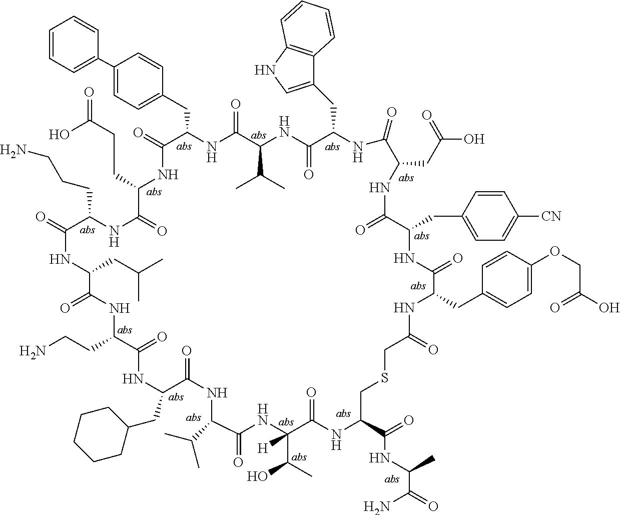 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C#N)cc2)NC(=O)[C@H](Cc2ccc(OCC(=O)O)cc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2231 was prepared on a 23 μmol scale. The yield of the product was 4.8 mg, and its estimated purity by LCMS analysis was 100%. Analysis condition A: Retention time=1.99 min; ESI-MS(+) m/z [M+2H]²⁺: 1010.1.

### Preparation of Compound 2232

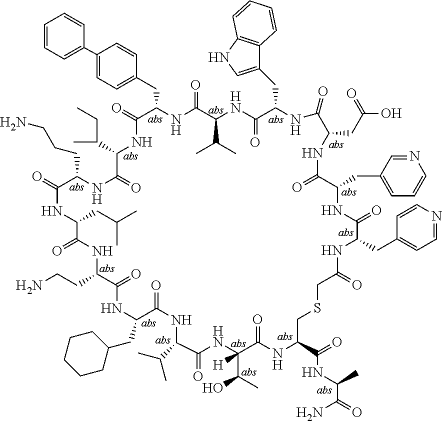 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2cccnc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2232 was prepared on a 23 μmol scale. The yield of the product was 3.7 mg, and its estimated purity by LCMS analysis was 94.2%. Analysis condition A: Retention time=1.77 min; ESI-MS(+) m/z [M+H]⁺: 1944.8.

### Preparation of Compound 2233

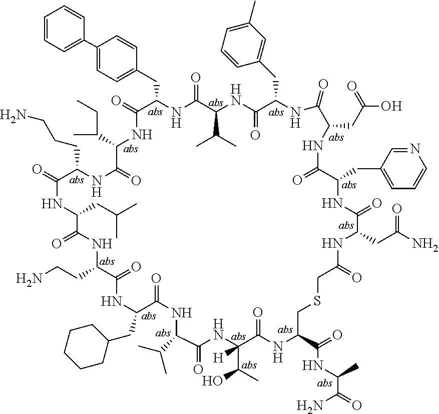 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2cccnc2)NC(=O)[C@H](CC(N)=O)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2233 was prepared on a 23 μmol scale. The yield of the product was 3.3 mg, and its estimated purity by LCMS analysis was 100%. Analysis condition A: Retention time=1.93 min; ESI-MS(+) m/z [M+H]⁺: 1886.2.

### Preparation of Compound 2234

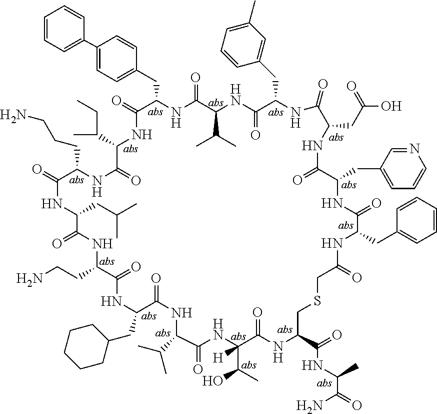 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2cccnc2)NC(=O)[C@H](Cc2ccccc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2234 was prepared on a 23 μmol scale. The yield of the product was 2.2 mg, and its estimated purity by LCMS analysis was 90.7%. Analysis condition B: Retention time=1.62 min; ESI-MS(+) m/z [M+H]⁺: 1919.1.

### Preparation of Compound 2235

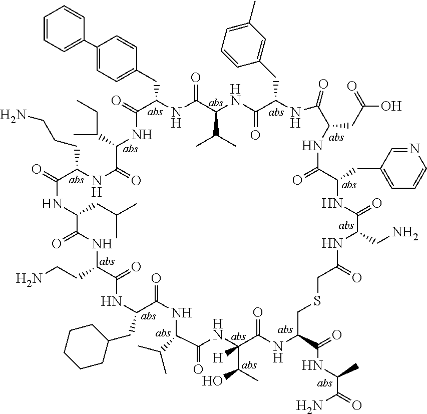 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2cccnc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2235 was prepared on a 23 μmol scale. The yield of the product was 8.3 mg, and its estimated purity by LCMS analysis was 82.8%. Analysis condition A: Retention time=1.76 min; ESI-MS(+) m/z [M+H]⁺: 1859.

### Preparation of Compound 2236

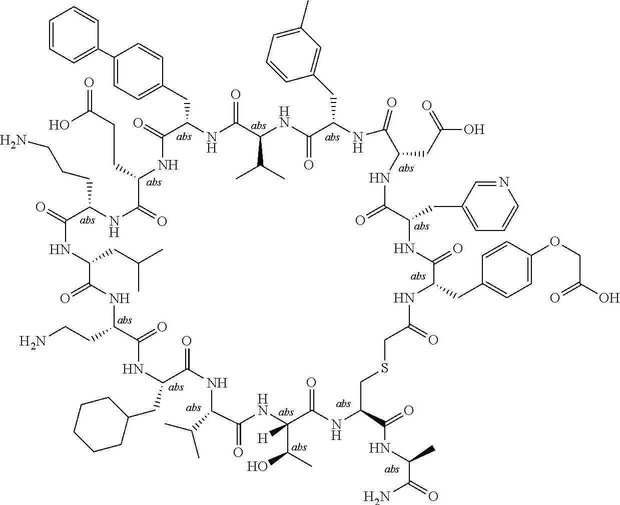 [SMILES: Cc1cccc(C[C@@H]2NC(=O)[C@H](CC(=O)O)NC(=O)[C@H](Cc3cccnc3)NC(=O)[C@H](Cc3ccc(OCC(=O)O)cc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC2=O)c1]

Compound 2236 was prepared on a 23 μmol scale. The yield of the product was 3.4 mg, and its estimated purity by LCMS analysis was 98.5%. Analysis condition A: Retention time=2.09 min; ESI-MS(+) m/z [M+H]⁺: 1992.9.

### Preparation of Compound 2237

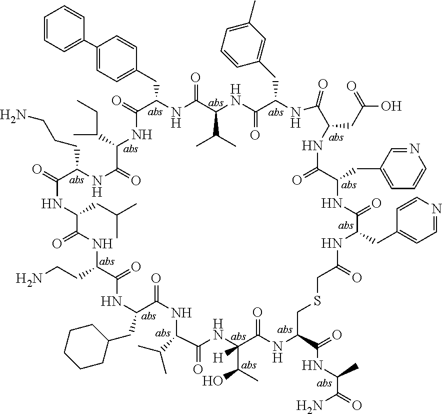 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2cccnc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2237 was prepared on a 23 μmol scale. The yield of the product was 2.1 mg, and its estimated purity by LCMS analysis was 100%. Analysis condition A: Retention time=2.02 min; ESI-MS(+) m/z [M+H]⁺: 1920.1.

### Preparation of Compound 2238

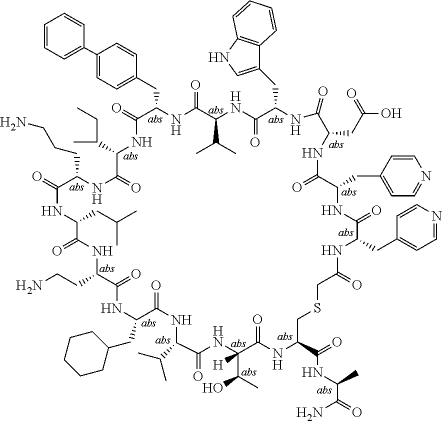 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2238 was prepared on a 23 μmol scale. The yield of the product was 4.3 mg, and its estimated purity by LCMS analysis was 91.3%. Analysis condition A: Retention time=1.92 min; ESI-MS(+) m/z [M+2H]²⁺: 973.2.

### Preparation of Compound 2239

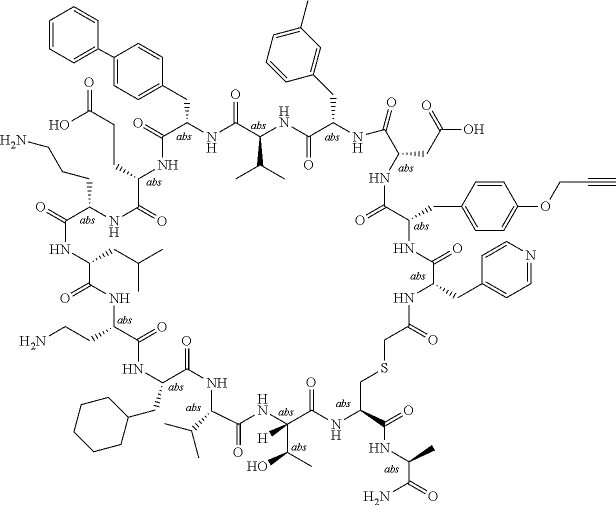 [SMILES: C#CCOc1ccc(C[C@@H]2NC(=O)[C@H](Cc3ccncc3)NC(=O)CSC[C@@H](C(=O)N[C@@H](C)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCC(=O)O)NC(=O)[C@H](Cc3ccc(-c4ccccc4)cc3)NC(=O)[C@H](C(C)C)NC(=O)[C@H](Cc3cccc(C)c3)NC(=O)[C@H](CC(=O)O)NC2=O)cc1]

Compound 2239 was prepared on a 21 μmol scale. The yield of the product was 5.2 mg, and its estimated purity by LCMS analysis was 90.6%. Analysis condition A: Retention time=2.18 min; ESI-MS(+) m/z [M+H]⁺: 1974.

### Preparation of Compound 2240

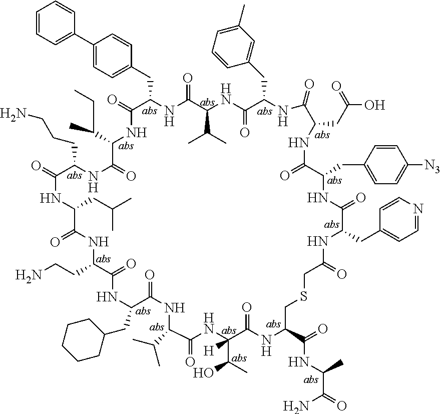 [SMILES: CC[C@H](C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(N=[N+]=[N-])cc2)NC(=O)[C@H](Cc2ccncc2)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2240 was prepared on a 24 μmol scale. The yield of the product was 1.8 mg, and its estimated purity by LCMS analysis was 81.7%. Analysis condition B: Retention time=1.7 min; ESI-MS(+) m/z [M+H]⁺: 1961.1.

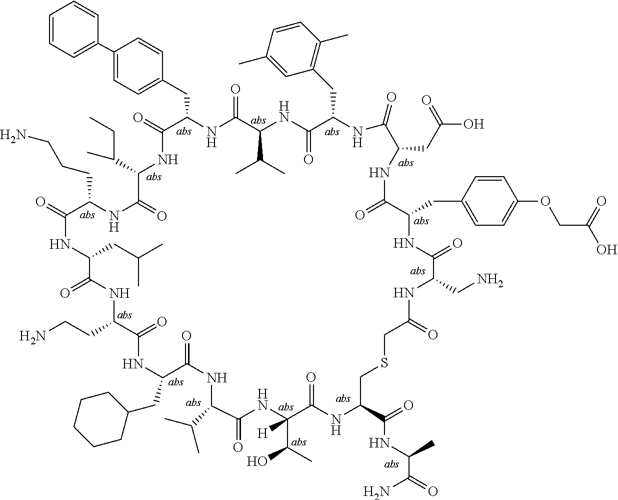 [SMILES: CCC(C)[C@@H]1NC(=O)[C@H](Cc2ccc(-c3ccccc3)cc2)NC(=O)[C@H](C(C)C)NC(=O)[C@H](Cc2cc(C)ccc2C)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2241 was prepared on a 23 μmol scale. The yield of the product was 1.5 mg, and its estimated purity by LCMS analysis was 100%. Analysis condition A: Retention time=1.72 min; ESI-MS(+) m/z [M+2H]²⁺: 973.1.

### Preparation of Compound 2242

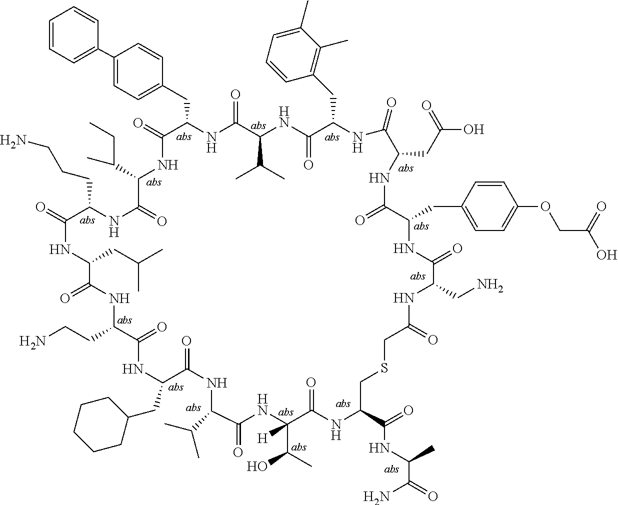 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2C)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2242 was prepared on a 23 μmol scale. The yield of the product was 1.4 mg, and its estimated purity by LCMS analysis was 93.9%. Analysis condition B: Retention time=1.56 min; ESI-MS(+) m/z [M+H]⁺: 1945.2.

### Preparation of Compound 2243

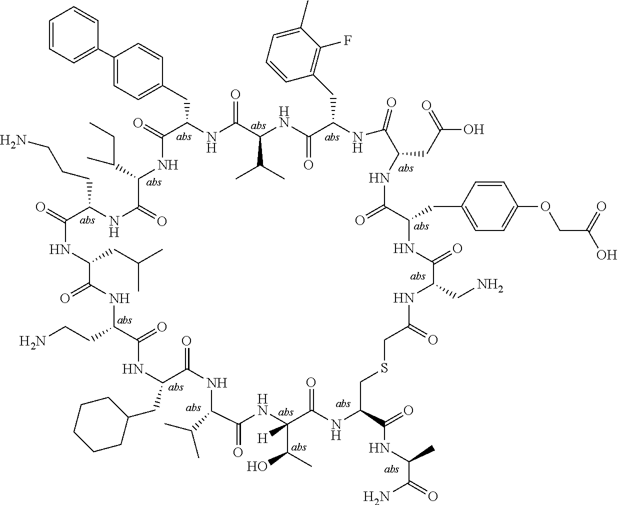 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2243 was prepared on a 23 μmol scale. The yield of the product was 1.5 mg, and its estimated purity by LCMS analysis was 93.9%. Analysis condition B: Retention time=1.53 min; ESI-MS(+) m/z [M+H]⁺: 1949.

### Preparation of Compound 2244

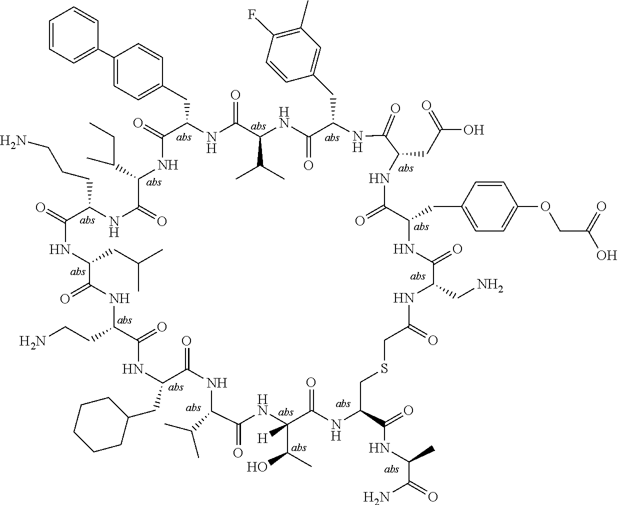 [SMILES: CCC(C)[C@@H]1NC(=O)[C@H](Cc2ccc(-c3ccccc3)cc2)NC(=O)[C@H](C(C)C)NC(=O)[C@H](Cc2ccc(F)c(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2244 was prepared on a 23 μmol scale. The yield of the product was 1.7 mg, and its estimated purity by LCMS analysis was 96.1%. Analysis condition B: Retention time=1.64 min; ESI-MS(+) m/z [M+H]⁺: 1948.8.

### Preparation of Compound 2245

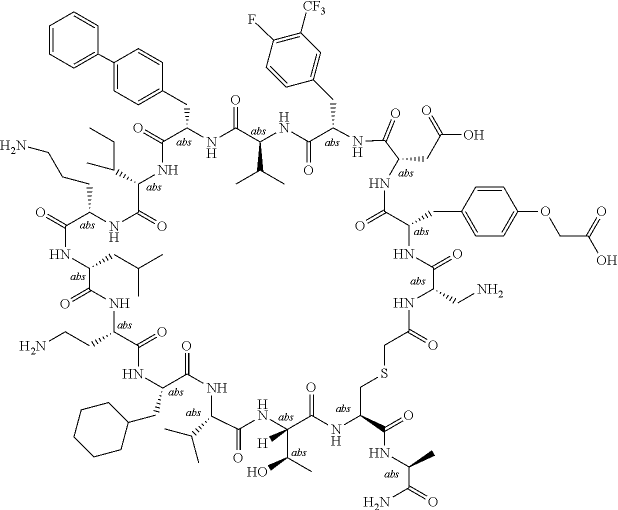 [SMILES: CCC(C)[C@@H]1NC(=O)[C@H](Cc2ccc(-c3ccccc3)cc2)NC(=O)[C@H](C(C)C)NC(=O)[C@H](Cc2ccc(F)c(C(F)(F)F)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2245 was prepared on a 23 μmol scale. The yield of the product was 2.5 mg, and its estimated purity by LCMS analysis was 100%. Analysis condition B: Retention time=1.6 min; ESI-MS(+) m/z [M+2H]²⁺: 1002.3.

### Preparation of Compound 2246

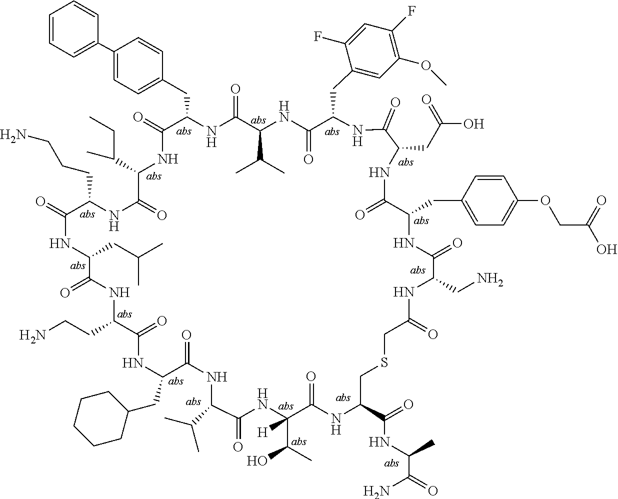 [SMILES: CCC(C)[C@@H]1NC(=O)[C@H](Cc2ccc(-c3ccccc3)cc2)NC(=O)[C@H](C(C)C)NC(=O)[C@H](Cc2cc(OC)c(F)cc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2246 was prepared on a 23 μmol scale. The yield of the product was 1.9 mg, and its estimated purity by LCMS analysis was 100%. Analysis condition A: Retention time=1.65 min; ESI-MS(+) m/z [M+2H]²⁺: 992.8.

### Preparation of Compound 2247

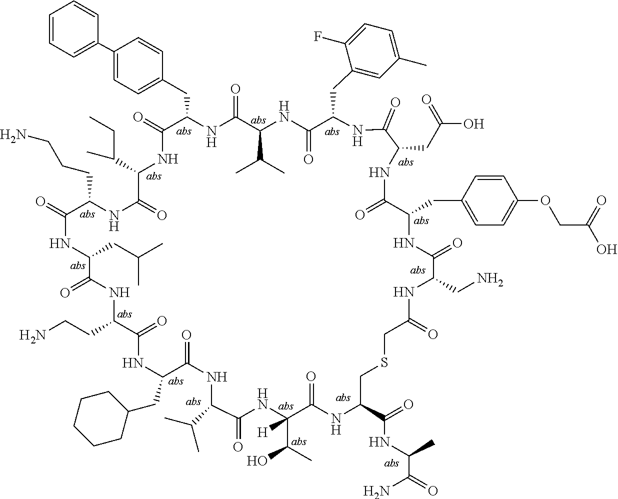 [SMILES: CCC(C)[C@@H]1NC(=O)[C@H](Cc2ccc(-c3ccccc3)cc2)NC(=O)[C@H](C(C)C)NC(=O)[C@H](Cc2cc(C)ccc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2247 was prepared on a 23 μmol scale. The yield of the product was 1 mg, and its estimated purity by LCMS analysis was 100%. Analysis condition A: Retention time=1.47 min; ESI-MS(+) m/z [M+2H]²⁺: 975.3.

### Preparation of Compound 2248

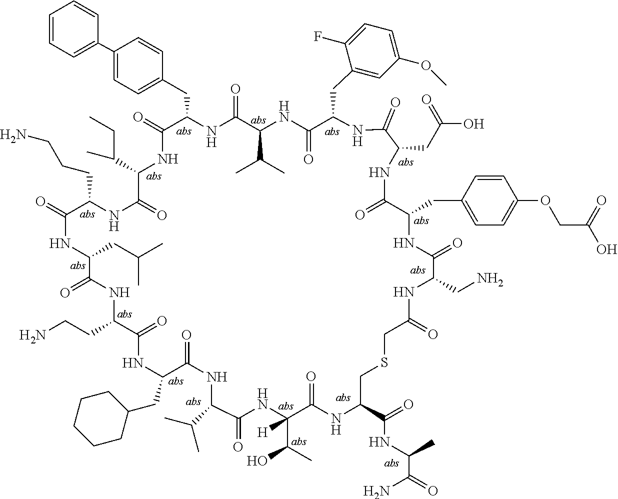 [SMILES: CCC(C)[C@@H]1NC(=O)[C@H](Cc2ccc(-c3ccccc3)cc2)NC(=O)[C@H](C(C)C)NC(=O)[C@H](Cc2cc(OC)ccc2F)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2248 was prepared on a 23 μmol scale. The yield of the product was 1.2 mg, and its estimated purity by LCMS analysis was 90%. Analysis condition A: Retention time=1.65 min; ESI-MS(+) m/z [M+2H]²⁺: 983.1.

### Preparation of Compound 2249

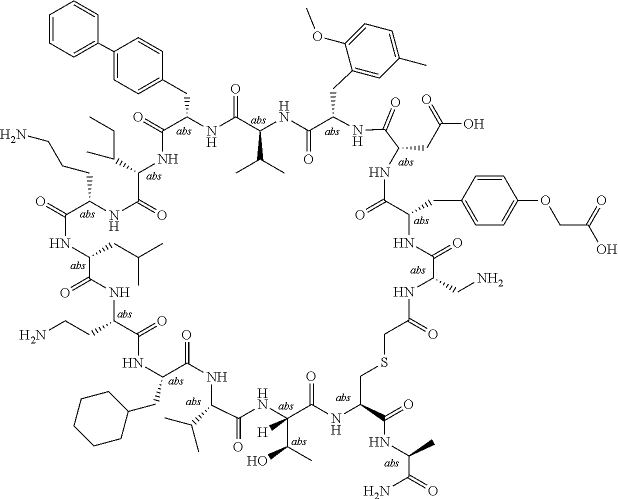 [SMILES: CCC(C)[C@@H]1NC(=O)[C@H](Cc2ccc(-c3ccccc3)cc2)NC(=O)[C@H](C(C)C)NC(=O)[C@H](Cc2cc(C)ccc2OC)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2249 was prepared on a 23 μmol scale. The yield of the product was 1.5 mg, and its estimated purity by LCMS analysis was 96.7%. Analysis condition A: Retention time=1.82 min; ESI-MS(+) m/z [M+H]⁺: 1960.8.

### Preparation of Compound 2250

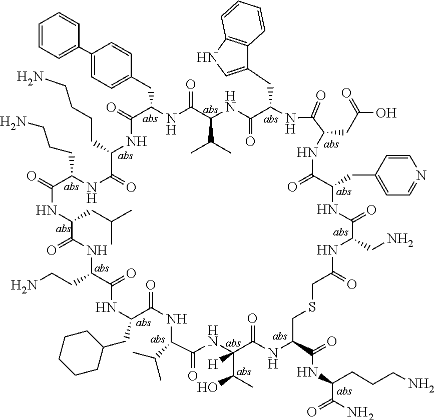 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CCCN)C(N)=O)NC(=O)[C@H]([C@@H](C)O)NC(=O)[C@H](C(C)C)NC(=O)[C@H](CC2CCCCC2)NC(=O)[C@H](CCN)NC1=O]

Compound 2250 was prepared on a 50 μmol scale. The yield of the product was 6.2 mg, and its estimated purity by LCMS analysis was 100%. Analysis condition A: Retention time=1.49 min; ESI-MS(+) m/z [M+H]⁺: 1940.8.

### Preparation of Compound 2251

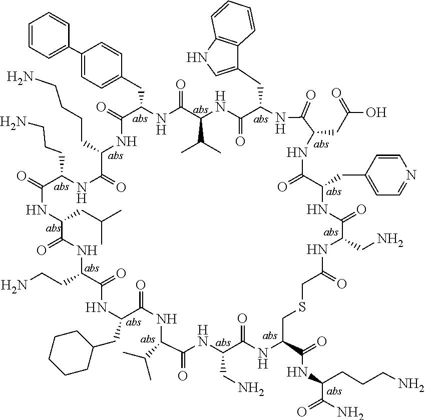 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CCCN)C(N)=O)NC(=O)[C@H](CN)NC(=O)[C@H](C(C)C)NC(=O)[C@H](CC2CCCCC2)NC(=O)[C@H](CCN)NC1=O]

Compound 2251 was prepared on a 50 μmol scale. The yield of the product was 4.7 mg, and its estimated purity by LCMS analysis was 95.5%. Analysis condition B: Retention time=1.24 min; ESI-MS(+) m/z [M+H]⁺: 1925.9.

### Preparation of Compound 2252

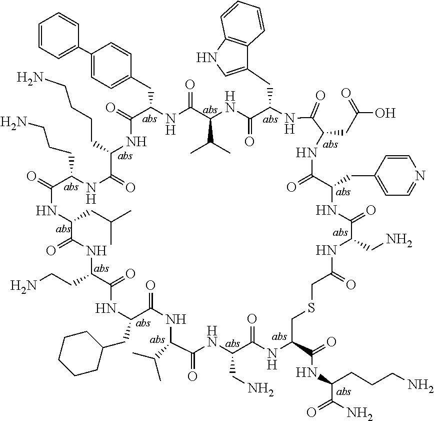 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CCCN)C(N)=O)NC(=O)[C@H](CN)NC(=O)[C@H](C(C)C)NC(=O)[C@H](CC2CCCCC2)NC(=O)[C@H](CCN)NC1=O]

Compound 2252 was prepared on a 50 μmol scale. The yield of the product was 4.9 mg, and its estimated purity by LCMS analysis was 100%. Analysis condition A: Retention time=1.52, 1.62 min; ESI-MS(+) m/z [M+2H]²⁺: 970.48, 970.44.

### Preparation of Compound 2253

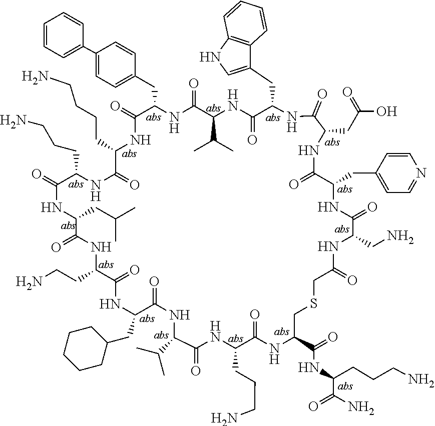 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CCCN)C(N)=O)NC(=O)[C@H](CCCN)NC(=O)[C@H](C(C)C)NC(=O)[C@H](CC2CCCCC2)NC(=O)[C@H](CCN)NC1=O]

Compound 2253 was prepared on a 50 μmol scale. The yield of the product was 1.5 mg, and its estimated purity by LCMS analysis was 94.6%. Analysis condition B: Retention time=1.13 min; ESI-MS(+) m/z [M+3H]³⁺: 652.

### Preparation of Compound 2254

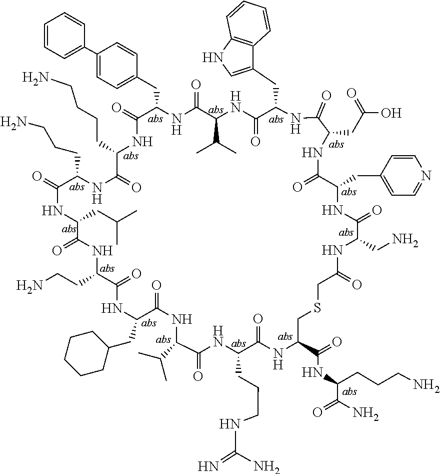 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CCCN)C(N)=O)NC(=O)[C@H](CCCNC(=N)N)NC(=O)[C@H](C(C)C)NC(=O)[C@H](CC2CCCCC2)NC(=O)[C@H](CCN)NC1=O]

Compound 2254 was prepared on a 50 μmol scale. The yield of the product was 5.9 mg, and its estimated purity by LCMS analysis was 97%. Analysis condition B: Retention time=1.23 min; ESI-MS(+) m/z [M+3H]³⁺: 666.3.

### Preparation of Compound 2255

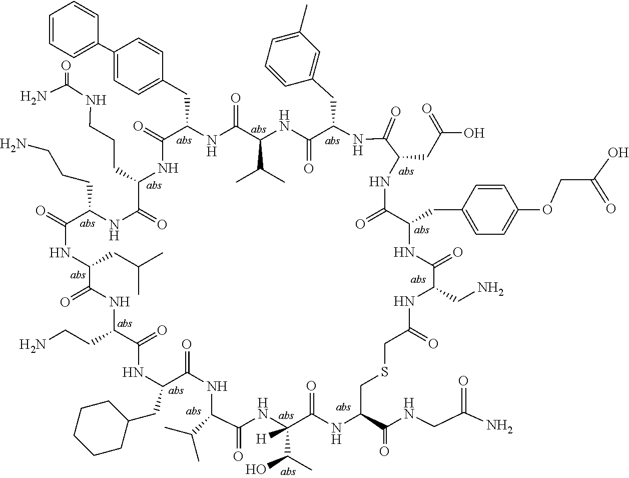 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)NCC(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2255 was prepared on a 50 μmol scale. The yield of the product was 14.3 mg, and its estimated purity by LCMS analysis was 91.7%. Analysis condition A: Retention time=1.56 min; ESI-MS(+) m/z [M+H]⁺: 1961.1.

### Preparation of Compound 2256

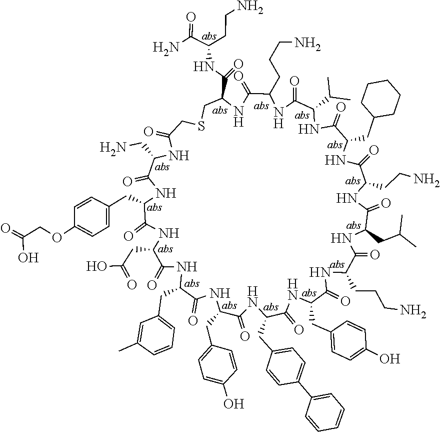 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)C(CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2256 was prepared on a 50 μmol scale. The yield of the product was 23.7 mg, and its estimated purity by LCMS analysis was 100%. Analysis condition A: Retention time=1.46 min; ESI-MS(+) m/z [M+2H]²⁺: 1044.3.

### Preparation of Compound 2257

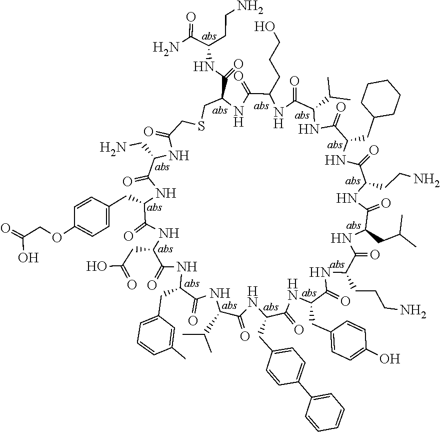 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)C(CCCO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2257 was prepared on a 50 μmol scale. The yield of the product was 13.9 mg, and its estimated purity by LCMS analysis was 94.6%. Analysis condition B: Retention time=1.45 min; ESI-MS(+) m/z [M+2H]²⁺: 1013.

### Preparation of Compound 2258

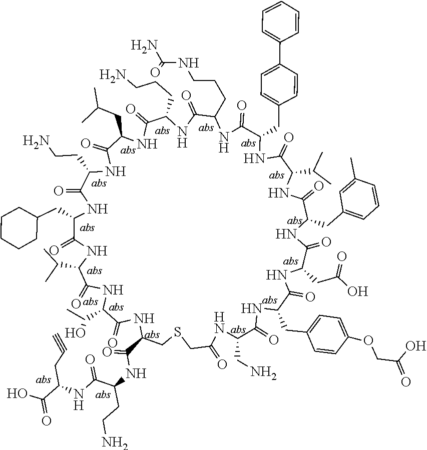 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)NC(CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O]

Compound 2258 was prepared on a 200 μmol scale. The yield of the product was 22.6 mg, and its estimated purity by LCMS analysis was 95.8%. Analysis condition: Retention time=1.54 min; ESI-MS(+) m/z [M+3H]³⁺: 701.1.

### Preparation of Compound 2259

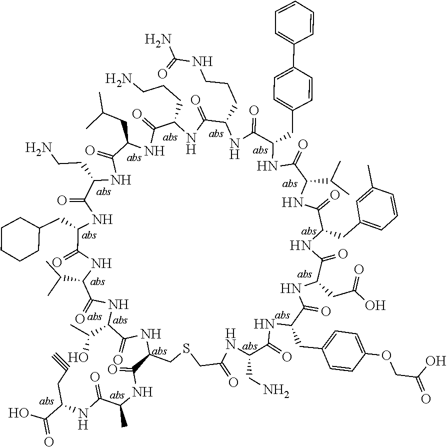 [SMILES: C#CC[C@H](NC(=O)[C@H](C)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O]

Compound 2259 was prepared on a 200 μmol scale. The yield of the product was 9.7 mg, and its estimated purity by LCMS analysis was 99.3%. Analysis condition: Retention time=1.58 min; ESI-MS(+) m/z [M+3H]³⁺: 691.2.

### Preparation of Compound 2260

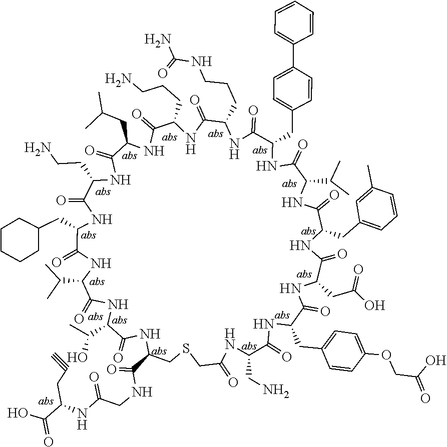 [SMILES: C#CC[C@H](NC(=O)CNC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O]

Compound 2260 was prepared on a 200 μmol scale. The yield of the product was 8 mg, and its estimated purity by LCMS analysis was 100%. Analysis condition: Retention time=1.61 min; ESI-MS(+) m/z [M+2H]²⁺: 1029.2.

### Preparation of Compound 2261

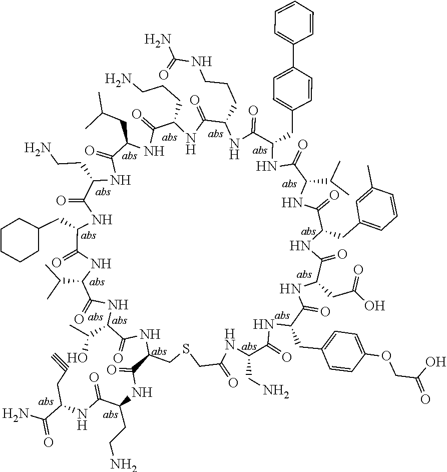 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(N)=O]

Compound 2261 was prepared on a 200 μmol scale. The yield of the product was 23.2 mg, and its estimated purity by LCMS analysis was 92.4%. Analysis condition: Retention time=1.58 min; ESI-MS(+) m/z [M+3H]³⁺: 700.5.

### Preparation of Compound 2262

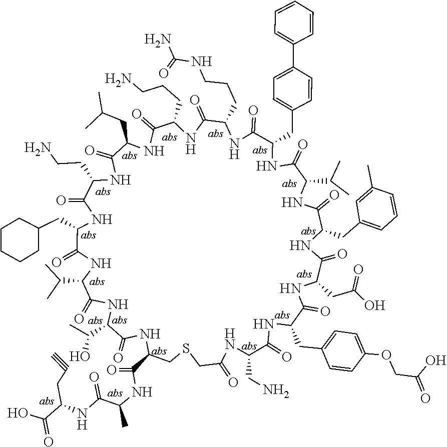 [SMILES: C#CC[C@H](NC(=O)[C@H](C)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O]

Compound 2262 was prepared on a 200 μmol scale. The yield of the product was 4.6 mg, and its estimated purity by LCMS analysis was 90.3%. Analysis condition B: Retention time=1.51 min; ESI-MS(+) m/z [M+2H]²⁺: 1036.2.

### Preparation of Compound 2263

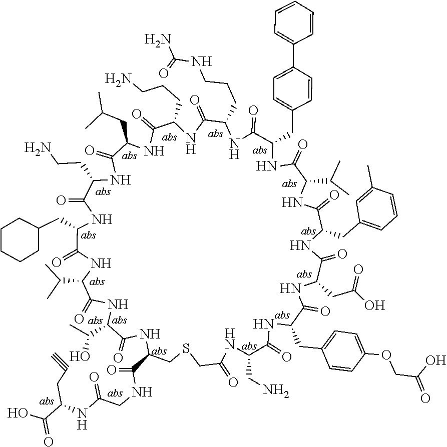 [SMILES: C#CC[C@H](NC(=O)CNC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](CCCNC(N)=O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H]([C@@H](C)O)C(=O)N1)C(=O)O]

Compound 2263 was prepared on a 200 μmol scale. The yield of the product was 4.7 mg, and its estimated purity by LCMS analysis was 90.7%. Analysis condition B: Retention time=1.47 min; ESI-MS(+) m/z [M+3H]³⁺: 686.

### Preparation of Compound 2264

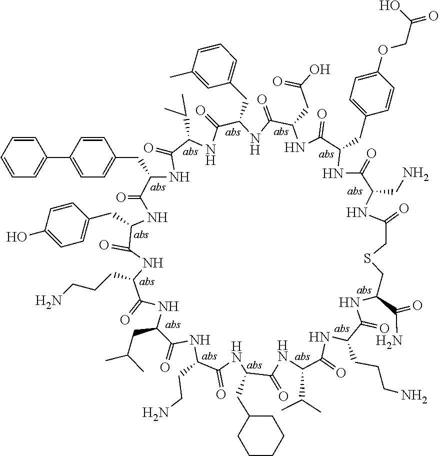 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2264 was prepared on a 50 μmol scale. The yield of the product was 27.2 mg, and its estimated purity by LCMS analysis was 96.7%. Analysis condition B: Retention time=1.54 min; ESI-MS(+) m/z [M+H]⁺: 1923.4.

### Preparation of Compound 2265

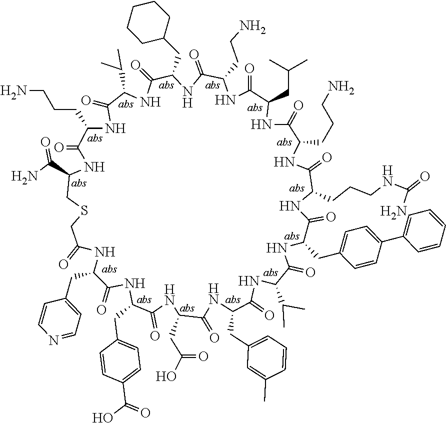 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2265 was prepared on a 50 μmol scale. The yield of the product was 11 mg, and its estimated purity by LCMS analysis was 97.9%. Analysis condition A: Retention time=1.5 min; ESI-MS(+) m/z [M+H]⁺: 1950.4.

### Preparation of Compound 2266

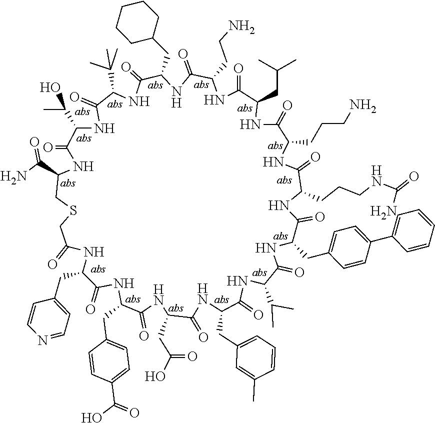 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H]([C@@H](C)O)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2266 was prepared on a 50 μmol scale. The yield of the product was 25.9 mg, and its estimated purity by LCMS analysis was 97.7%. Analysis condition A: Retention time=1.6 min; ESI-MS(+) m/z [M+H]⁺: 1951.2.

### Preparation of Compound 2267

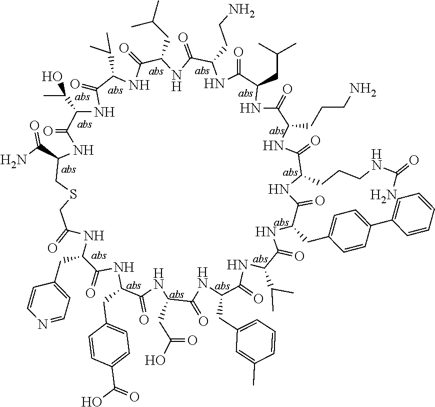 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H]([C@@H](C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2267 was prepared on a 50 μmol scale. The yield of the product was 18.6 mg, and its estimated purity by LCMS analysis was 92.8%. Analysis condition A: Retention time=1.47 min; ESI-MS(+) m/z [M+H]⁺: 1896.3.

### Preparation of Compound 2268

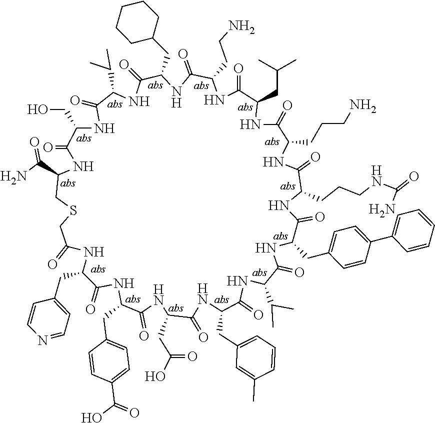 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2268 was prepared on a 50 μmol scale. The yield of the product was 10.2 mg, and its estimated purity by LCMS analysis was 92.9%. Analysis condition A: Retention time=1.55 min; ESI-MS(+) m/z [M+H]⁺: 1923.3.

### Preparation of Compound 2269

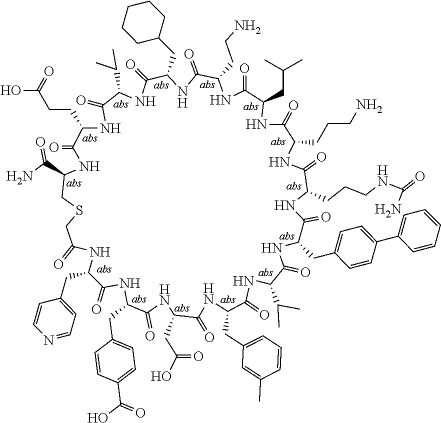 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2269 was prepared on a 50 μmol scale. The yield of the product was 12.2 mg, and its estimated purity by LCMS analysis was 94.2%. Analysis condition A: Retention time=1.48 min; ESI-MS(+) m/z [M+H]⁺: 1964.1.

### Preparation of Compound 2270

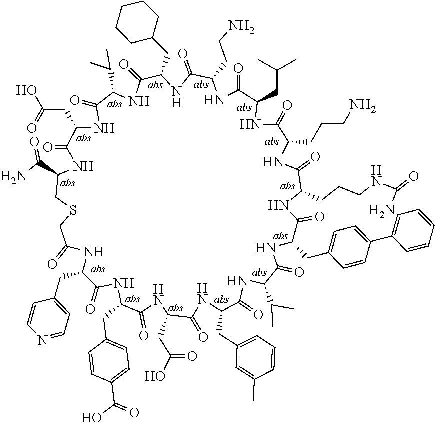 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2270 was prepared on a 50 μmol scale. The yield of the product was 14.1 mg, and its estimated purity by LCMS analysis was 98.2%. Analysis condition B: Retention time=1.5 min; ESI-MS(+) m/z [M+H]⁺: 1950.2.

### Preparation of Compound 2271

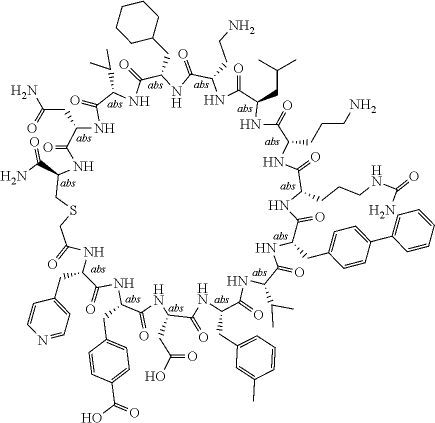 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2271 was prepared on a 50 μmol scale. The yield of the product was 11.9 mg, and its estimated purity by LCMS analysis was 96.6%. Analysis condition A: Retention time=1.53 min; ESI-MS(+) m/z [M+H]⁺: 1949.2.

### Preparation of Compound 2272

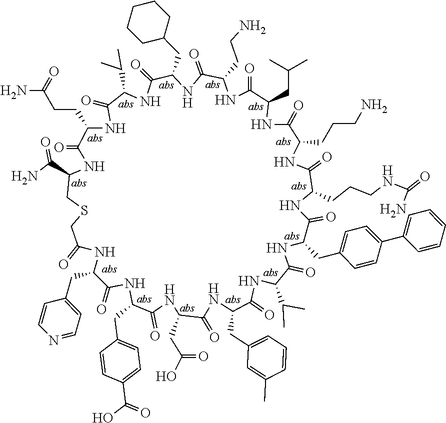 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2272 was prepared on a 50 μmol scale. The yield of the product was 14.9 mg, and its estimated purity by LCMS analysis was 97.5%. Analysis condition A: Retention time=1.52 min; ESI-MS(+) m/z [M+H]⁺: 1963.

### Preparation of Compound 2273

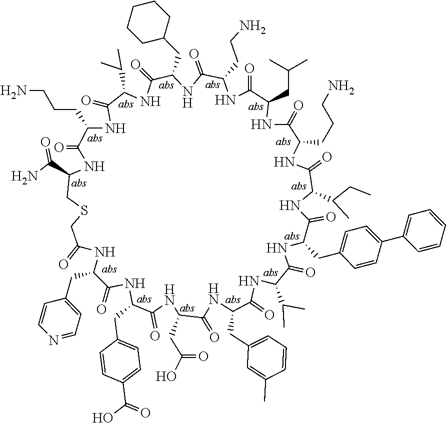 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CCCN)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2273 was prepared on a 50 μmol scale. The yield of the product was 29.3 mg, and its estimated purity by LCMS analysis was 95%. Analysis condition A: Retention time=1.59 min; ESI-MS(+) m/z [M+3H]³⁺: 636.1.

### Preparation of Compound 2274

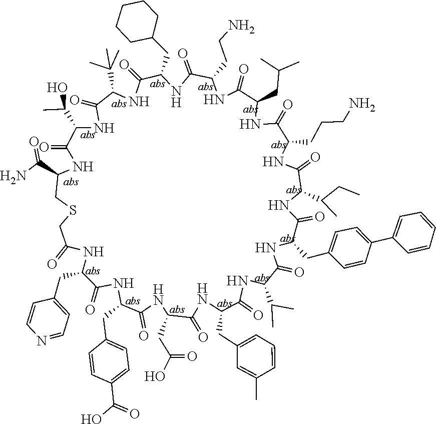 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H]([C@@H](C)O)NC(=O)[C@H](C(C)(C)C)NC(=O)[C@H](CC2CCCCC2)NC(=O)[C@H](CCN)NC(=O)[C@@H](CC(C)C)NC(=O)[C@H](CCCN)NC1=O]

Compound 2274 was prepared on a 50 μmol scale. The yield of the product was 27.8 mg, and its estimated purity by LCMS analysis was 93%. Analysis condition A: Retention time=1.89 min; ESI-MS(+) m/z [M+H]⁺: 1906.

### Preparation of Compound 2275

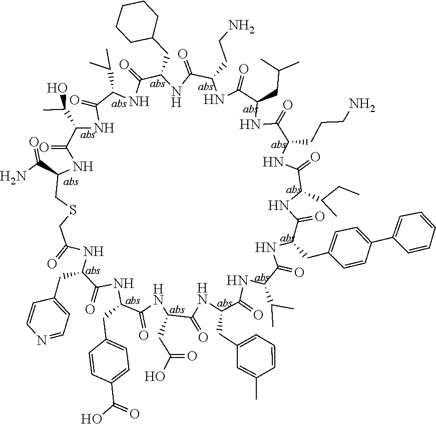 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2275 was prepared on a 50 μmol scale. The yield of the product was 22.1 mg, and its estimated purity by LCMS analysis was 92.9%. Analysis condition A: Retention time=1.74 min; ESI-MS(+) m/z [M+2H]²⁺: 947.2.

### Preparation of Compound 2276

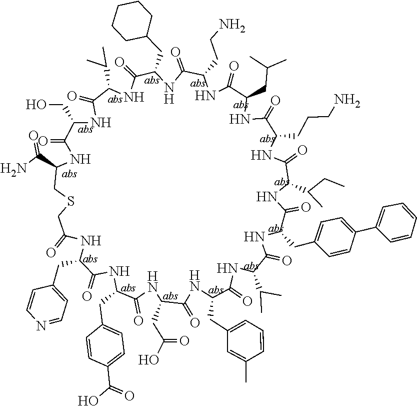 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2276 was prepared on a 50 μmol scale. The yield of the product was 5.2 mg, and its estimated purity by LCMS analysis was 94%. Analysis condition A: Retention time=1.69 min; ESI-MS(+) m/z [M+2H]²⁺: 940.

### Preparation of Compound 2277

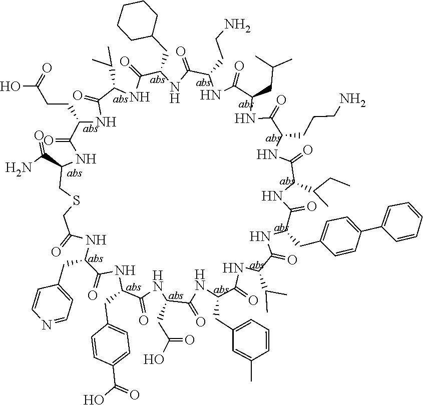 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2277 was prepared on a 50 μmol scale. The yield of the product was 13.6 mg, and its estimated purity by LCMS analysis was 92.5%. Analysis condition B: Retention time=1.57 min; ESI-MS(+) m/z [M+H]⁺: 1919.9.

### Preparation of Compound 2278

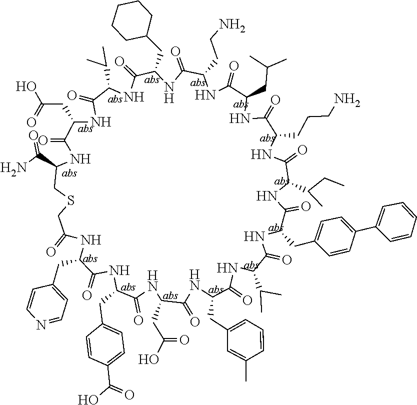 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2278 was prepared on a 50 μmol scale. The yield of the product was 17.8 mg, and its estimated purity by LCMS analysis was 96.2%. Analysis condition A: Retention time=1.75 min; ESI-MS(+) m/z [M+H]⁺: 1905.6.

### Preparation of Compound 2279

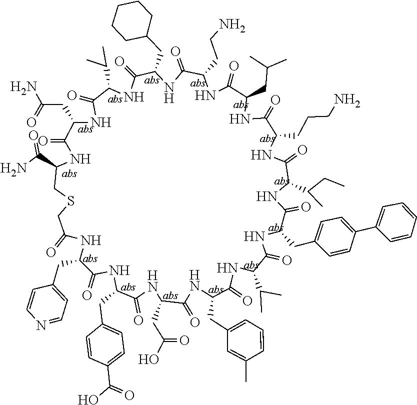 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2279 was prepared on a 50 μmol scale. The yield of the product was 7 mg, and its estimated purity by LCMS analysis was 96.8%. Analysis condition B: Retention time=1.57 min; ESI-MS(+) m/z [M+H]⁺: 1905.3.

### Preparation of Compound 2280

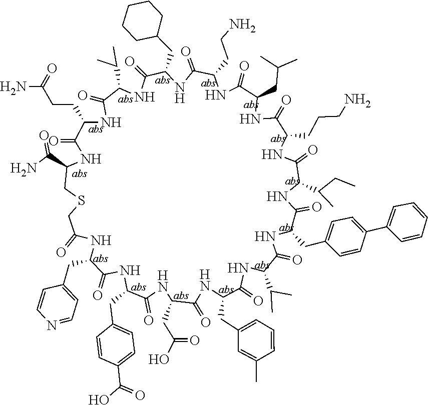 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(N)=O)NC(=O)[C@H](CCC(N)=O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2280 was prepared on a 50 μmol scale. The yield of the product was 12.3 mg, and its estimated purity by LCMS analysis was 95.8%. Analysis condition A: Retention time=1.65 min; ESI-MS(+) m/z [M+2H]²⁺: 960.1.

### Preparation of Compound 2281

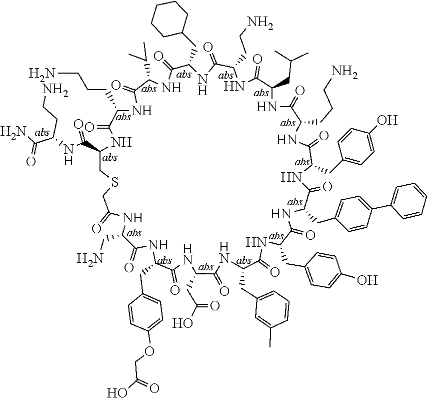 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2281 was prepared on a 50 μmol scale. The yield of the product was 27.5 mg, and its estimated purity by LCMS analysis was 95.7%. Analysis condition: Retention time=1.47 min; ESI-MS(+) m/z [M+3H]³⁺: 675.2.

### Preparation of Compound 2282

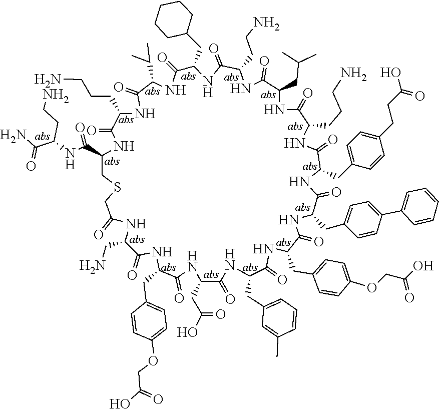 [SMILES: Cc1cccc(C[C@@H]2NC(=O)[C@H](CC(=O)O)NC(=O)[C@H](Cc3ccc(OCC(=O)O)cc3)NC(=O)[C@H](CN)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(CCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2282 was prepared on a 50 μmol scale. The yield of the product was 5.2 mg, and its estimated purity by LCMS analysis was 95%. Analysis condition B: Retention time=1.43 min; ESI-MS(+) m/z [M+2H]²⁺: 157.2.

### Preparation of Compound 2283

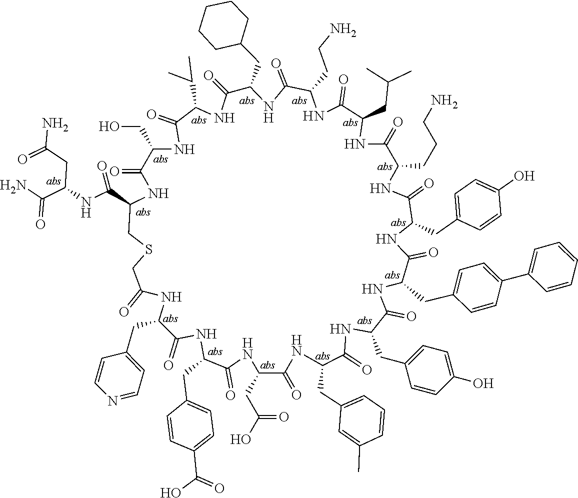 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2283 was prepared on a 50 μmol scale. The yield of the product was 43.3 mg, and its estimated purity by LCMS analysis was 98.9%. Analysis condition B: Retention time=1.54 min; ESI-MS(+) m/z [M+2H]²⁺: 1060.9.

### Preparation of Compound 2284

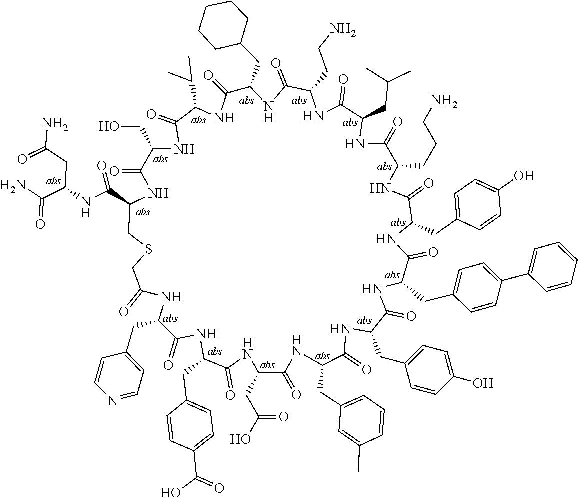 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2284 was prepared on a 50 μmol scale. The yield of the product was 50.8 mg, and its estimated purity by LCMS analysis was 95%. Analysis condition B: Retention time=1.51 min; ESI-MS(+) m/z [M+2H]²⁺: 1054.1.

### Preparation of Compound 2285

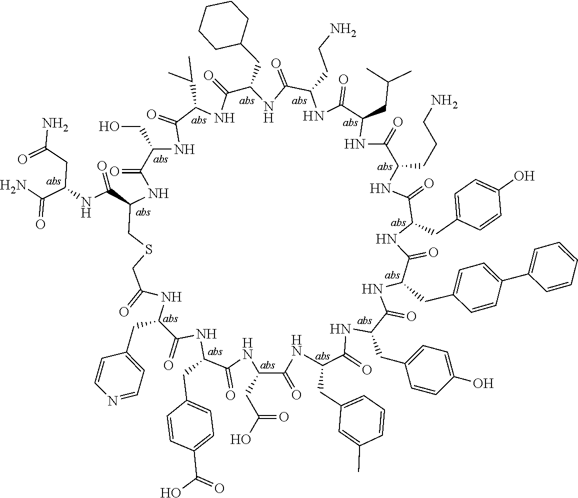 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2285 was prepared on a 50 μmol scale. The yield of the product was 54.4 mg, and its estimated purity by LCMS analysis was 96%. Analysis condition B: Retention time=1.54 min; ESI-MS(+) m/z [M+2H]²⁺: 1061.1.

### Preparation of Compound 2286

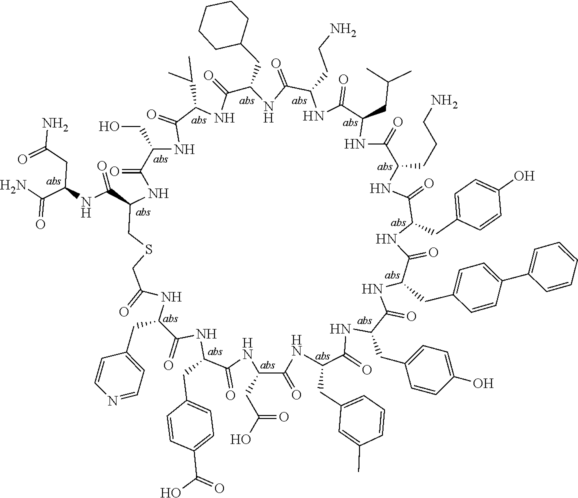 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2286 was prepared on a 50 μmol scale. The yield of the product was 43.2 mg, and its estimated purity by LCMS analysis was 96.6%. Analysis condition B: Retention time=1.52 min; ESI-MS(+) m/z [M+2H]²⁺: 1054.1.

### Preparation of Compound 2287

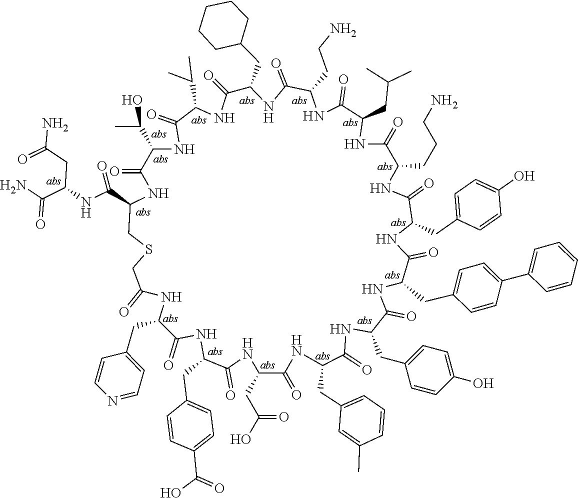 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(O)cc3)NC2=O)c1]

Compound 2287 was prepared on a 50 μmol scale. The yield of the product was 32 mg, and its estimated purity by LCMS analysis was 98.6%. Analysis condition B: Retention time=1.58 min; ESI-MS(+) m/z [M+2H]²⁺: 1054.1.

### Preparation of Compound 2288

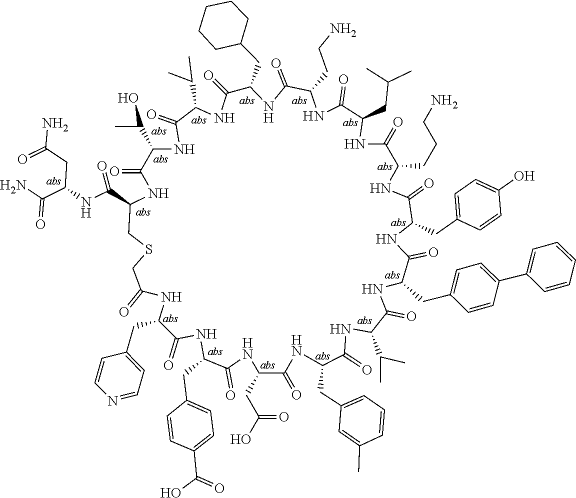

Compound 2288 was prepared on a 50 μmol scale. The yield of the product was 57 mg, and its estimated purity by LCMS analysis was 95%. Analysis condition A: Retention time=1.65 min; ESI-MS(+) m/z [M+2H]²⁺: 1028.96.

### Preparation of Compound 2289

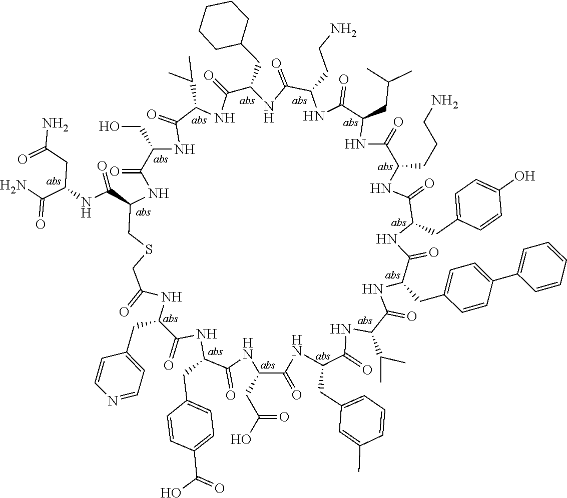 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2289 was prepared on a 50 μmol scale. The yield of the product was 18.4 mg, and its estimated purity by LCMS analysis was 95.9%. Analysis condition B: Retention time=1.58 min; ESI-MS(+) m/z [M+2H]²⁺: 1022.1.

### Preparation of Compound 2290

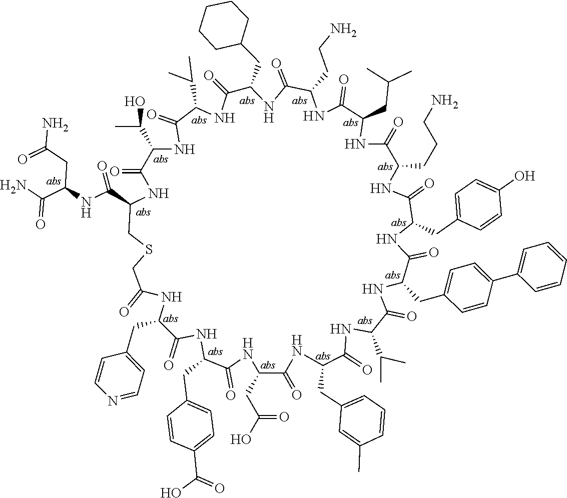 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2290 was prepared on a 50 μmol scale. The yield of the product was 32.4 mg, and its estimated purity by LCMS analysis was 95.9%. Analysis condition B: Retention time=1.64 min; ESI-MS(+) m/z [M+2H]²⁺: 1029.

### Preparation of Compound 2291

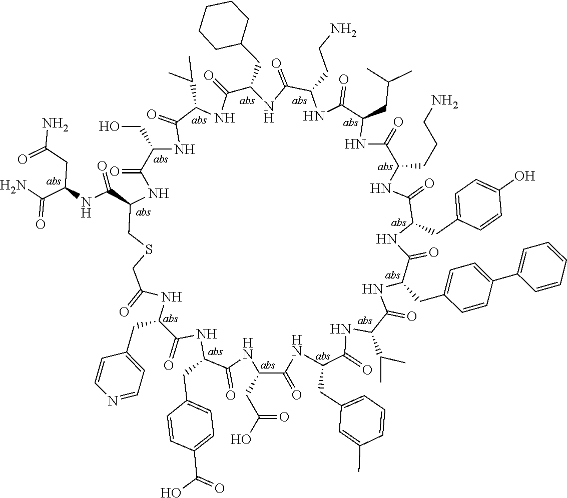 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2291 was prepared on a 50 μmol scale. The yield of the product was 27.4 mg, and its estimated purity by LCMS analysis was 93.4%. Analysis condition B: Retention time=1.61 min; ESI-MS(+) m/z [M+2H]²⁺: 1022.

### Preparation of Compound 2292

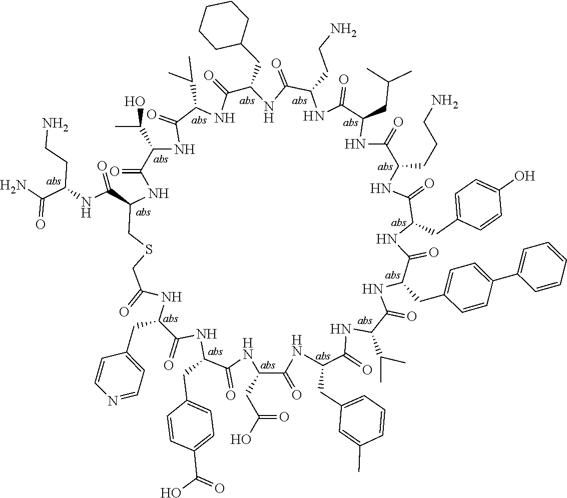 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](C(C)C)NC2=O)c1]

Compound 2292 was prepared on a 50 μmol scale. The yield of the product was 20.9 mg, and its estimated purity by LCMS analysis was 96.6%. Analysis condition B: Retention time=1.55 min; ESI-MS(+) m/z [M+2H]²⁺: 1022.

### Preparation of Compound 2293

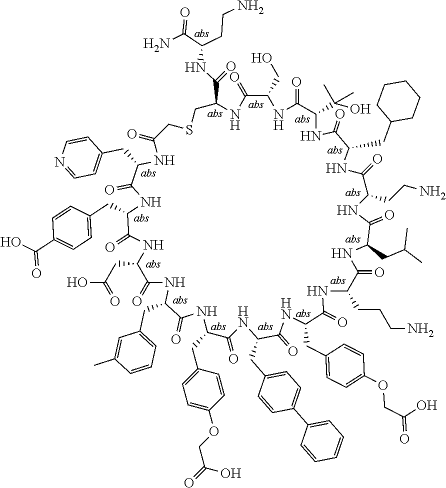 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2293 was prepared on a 50 μmol scale. The yield of the product was 13.7 mg, and its estimated purity by LCMS analysis was 93.4%. Analysis condition B: Retention time=1.55, 1.59 min; ESI-MS(+) m/z [M+2H]²⁺: 1112.94, 1112.94.

### Preparation of Compound 2294

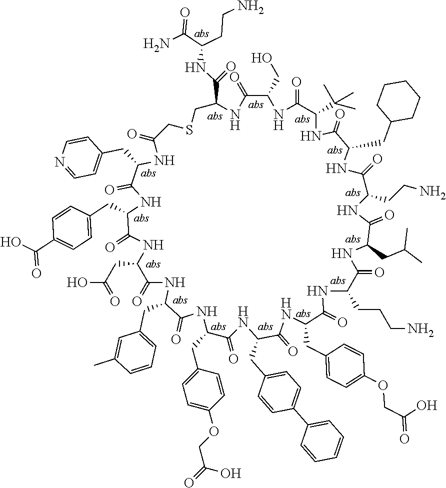 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2294 was prepared on a 50 μmol scale. The yield of the product was 11.4 mg, and its estimated purity by LCMS analysis was 97.8%. Analysis condition B: Retention time=1.5 min; ESI-MS(+) m/z [M+2H]²⁺: 1112.2.

### Preparation of Compound 2295

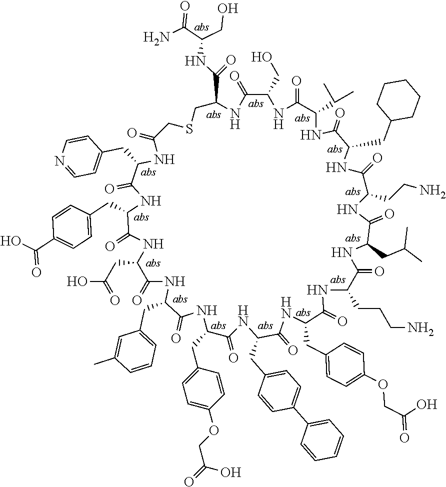 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CO)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2295 was prepared on a 50 μmol scale. The yield of the product was 8.4 mg, and its estimated purity by LCMS analysis was 96.6%. Analysis condition B: Retention time=1.55 min; ESI-MS(+) m/z [M+2H]²⁺: 1098.3.

### Preparation of Compound 2296

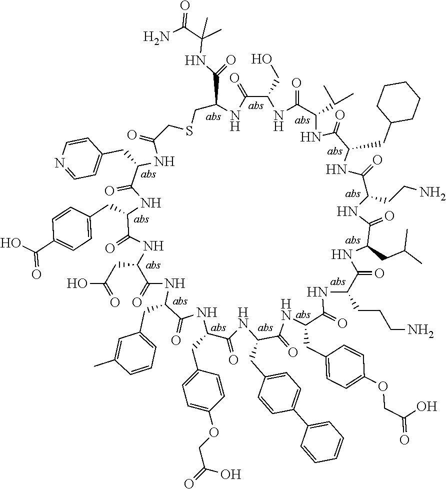 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)NC(C)(C)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2296 was prepared on a 50 μmol scale. The yield of the product was 29.1 mg, and its estimated purity by LCMS analysis was 97.3%. Analysis condition B: Retention time=1.57 min; ESI-MS(+) m/z [M+3H]³⁺: 732.2.

### Preparation of Compound 2297

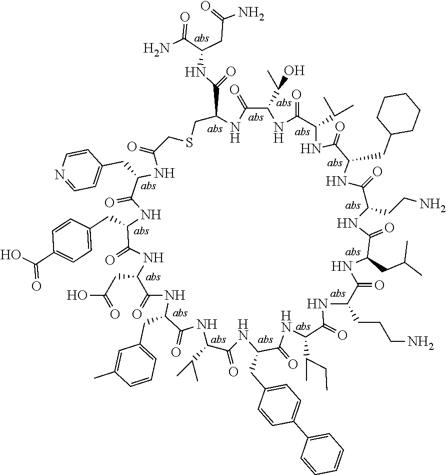 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2297 was prepared on a 50 μmol scale. The yield of the product was 5.6 mg, and its estimated purity by LCMS analysis was 98.1%. Analysis condition A: Retention time=1.83 min; ESI-MS(+) m/z [M+2H]²⁺: 1003.8.

### Preparation of Compound 2298

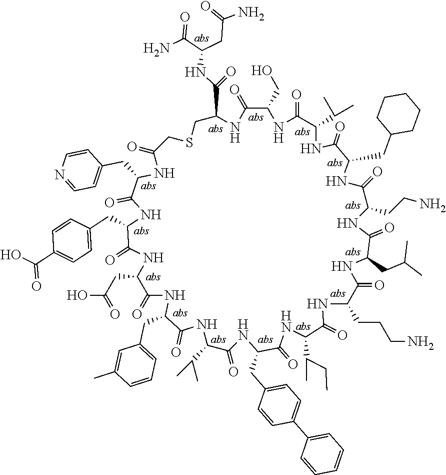 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2298 was prepared on a 50 μmol scale. The yield of the product was 1.9 mg, and its estimated purity by LCMS analysis was 95.6%. Analysis condition A: Retention time=1.58, 1.62 min; ESI-MS(+) m/z [M+H]⁺: 1993.14, 1993.14.

### Preparation of Compound 2299

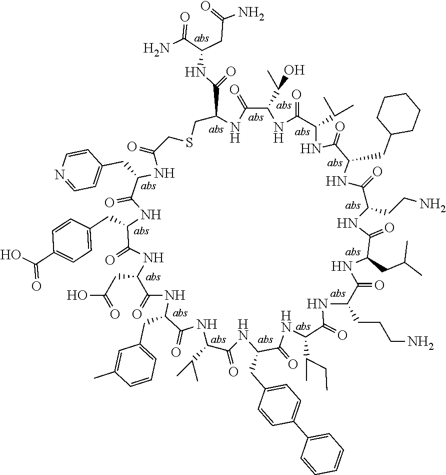 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2299 was prepared on a 50 μmol scale. The yield of the product was 14.7 mg, and its estimated purity by LCMS analysis was 94.6%. Analysis condition B: Retention time=1.86 min; ESI-MS(+) m/z [M+2H]²⁺: 1004.1.

### Preparation of Compound 2300

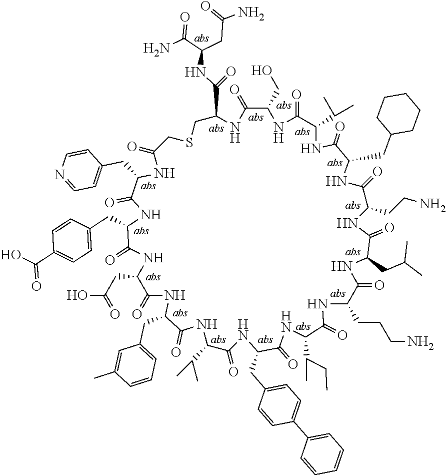 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2300 was prepared on a 50 μmol scale. The yield of the product was 3.2 mg, and its estimated purity by LCMS analysis was 98%. Analysis condition B: Retention time=1.76 min; ESI-MS(+) m/z [M+2H]²⁺: 1993.2.

### Preparation of Compound 2301

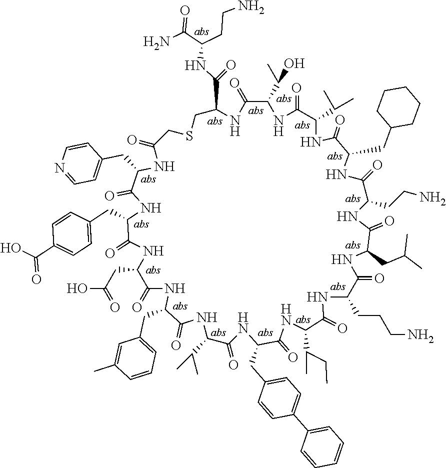 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2301 was prepared on a 50 μmol scale. The yield of the product was 14.5 mg, and its estimated purity by LCMS analysis was 95%. Analysis condition B: Retention time=1.5 min; ESI-MS(+) m/z [M+2H]²⁺: 1992.3.

### Preparation of Compound 2302

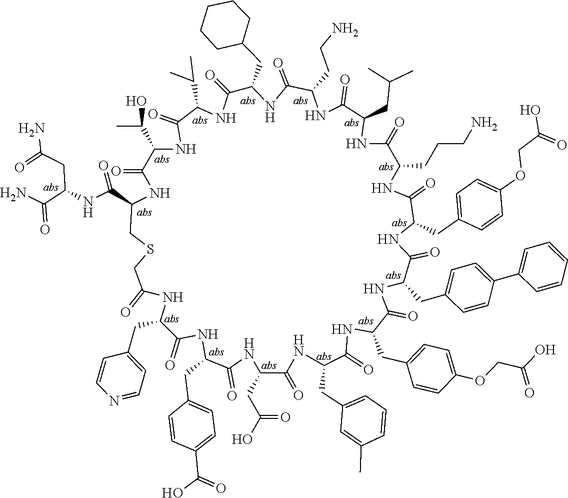 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2302 was prepared on a 50 μmol scale. The yield of the product was 38 mg, and its estimated purity by LCMS analysis was 94.7%. Analysis condition A: Retention time=1.29 min; ESI-MS(+) m/z [M+2H]²⁺: 1119.

### Preparation of Compound 2303

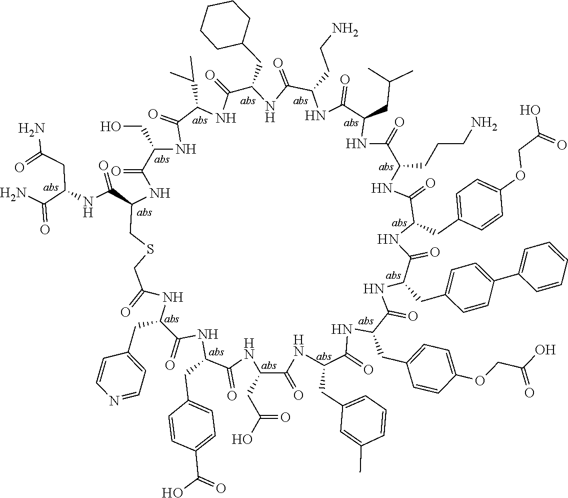 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2303 was prepared on a 50 μmol scale. The yield of the product was 38.6 mg, and its estimated purity by LCMS analysis was 92.5%. Analysis condition B: Retention time=1.54 min; ESI-MS(+) m/z [M+2H]²⁺: 1112.1.

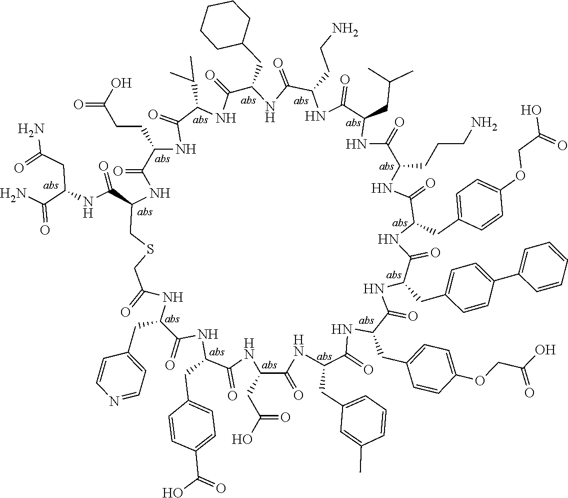 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2304 was prepared on a 50 μmol scale. The yield of the product was 9.1 mg, and its estimated purity by LCMS analysis was 100%. Analysis condition A: Retention time=1.32 min; ESI-MS(+) m/z [M+2H]²⁺: 1133.

### Preparation of Compound 2305

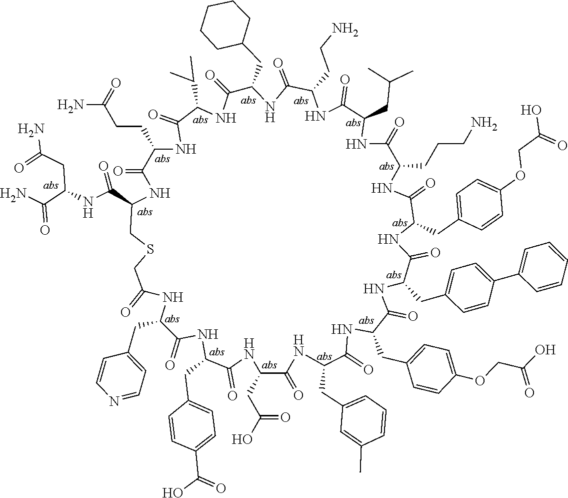 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CC(N)=O)C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2305 was prepared on a 50 μmol scale. The yield of the product was 8.2 mg, and its estimated purity by LCMS analysis was 100%. Analysis condition A: Retention time=1.3 min; ESI-MS(+) m/z [M+2H]²⁺: 1132.

### Preparation of Compound 2306

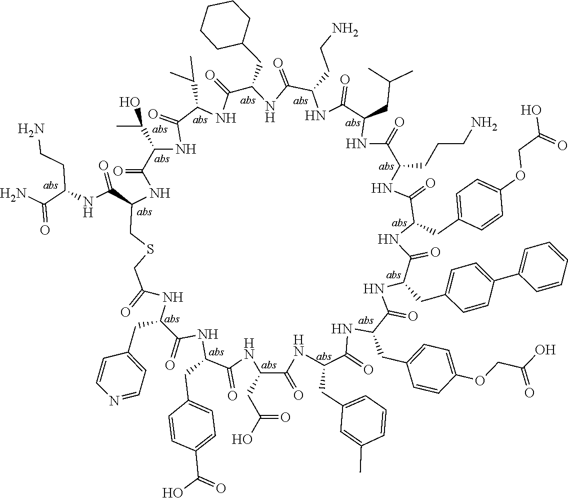 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2306 was prepared on a 50 μmol scale. The yield of the product was 11.7 mg, and its estimated purity by LCMS analysis was 93.6%. Analysis condition B: Retention time=1.56 min; ESI-MS(+) m/z [M+2H]²⁺: 1112.1.

### Preparation of Compound 2307

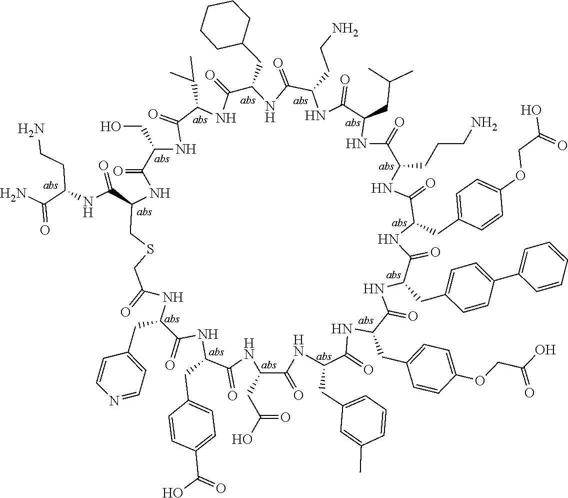 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2307 was prepared on a 50 μmol scale. The yield of the product was 7.3 mg, and its estimated purity by LCMS analysis was 95.6%. Analysis condition B Retention time=1.53 min; ESI-MS(+) m/z [M+2H]²⁺: 1105.1.

### Preparation of Compound 2308

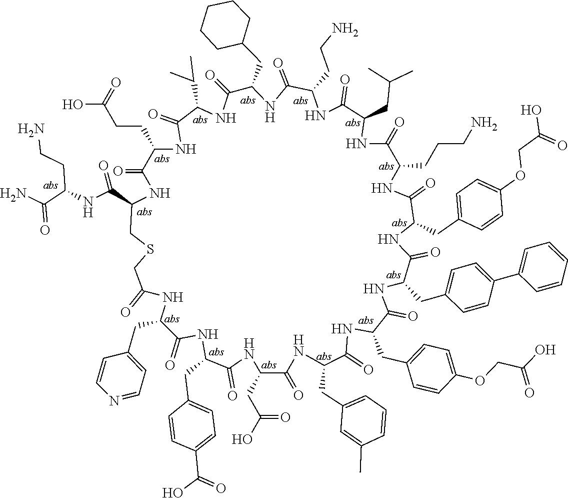 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2308 was prepared on a 50 μmol scale. The yield of the product was 5.4 mg, and its estimated purity by LCMS analysis was 99.1%. Analysis condition B: Retention time=1.57 min; ESI-MS(+) m/z [M+3H]³⁺: 751.3.

### Preparation of Compound 2309

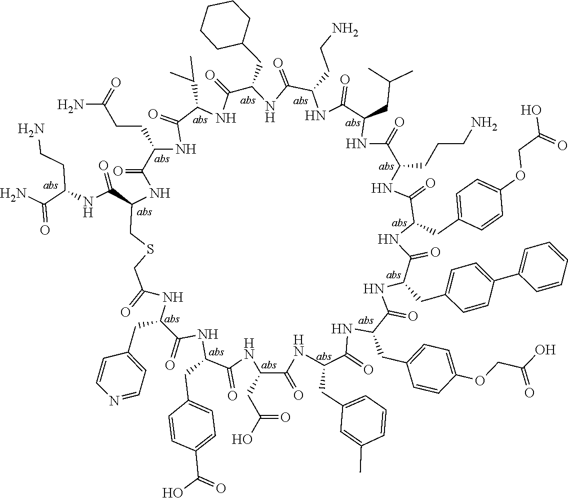 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2309 was prepared on a 50 μmol scale. The yield of the product was 4 mg, and its estimated purity by LCMS analysis was 94.1%. Analysis condition B: Retention time=1.5 min; ESI-MS(+) m/z [M+2H]²⁺: 1125.

### Preparation of Compound 2310

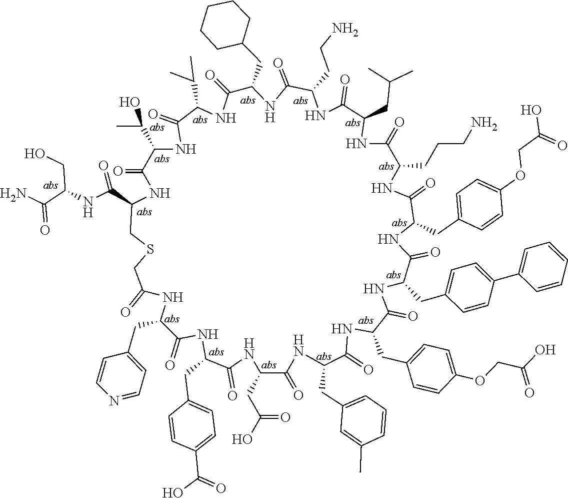 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CO)C(N)=O)NC(=O)[C@H]([C@@H](C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2310 was prepared on a 50 μmol scale. The yield of the product was 16 mg, and its estimated purity by LCMS analysis was 96.4%. Analysis condition B: Retention time=1.6 min; ESI-MS(+) m/z [M+2H]²⁺: 1105.2.

### Preparation of Compound 2311

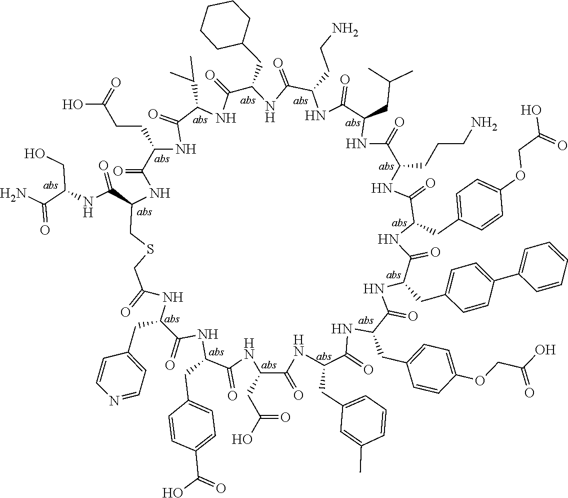 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CO)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2311 was prepared on a 50 μmol scale. The yield of the product was 7.7 mg, and its estimated purity by LCMS analysis was 97.4%. Analysis condition A: Retention time=1.34 min; ESI-MS(+) m/z [M+2H]²⁺: 1119.

### Preparation of Compound 2312

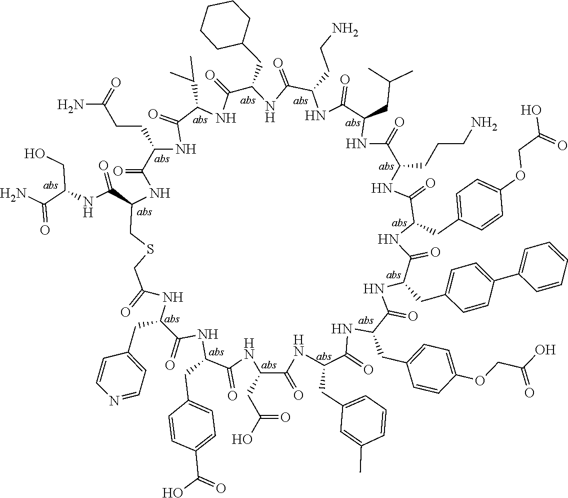 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CO)C(N)=O)NC(=O)[C@H](CCC(N)=O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2312 was prepared on a 50 μmol scale. The yield of the product was 3.8 mg, and its estimated purity by LCMS analysis was 100%. Analysis condition A: Retention time=1.4 min; ESI-MS(+) m/z [M+2H]²⁺: 1119.1.

### Preparation of Compound 2313

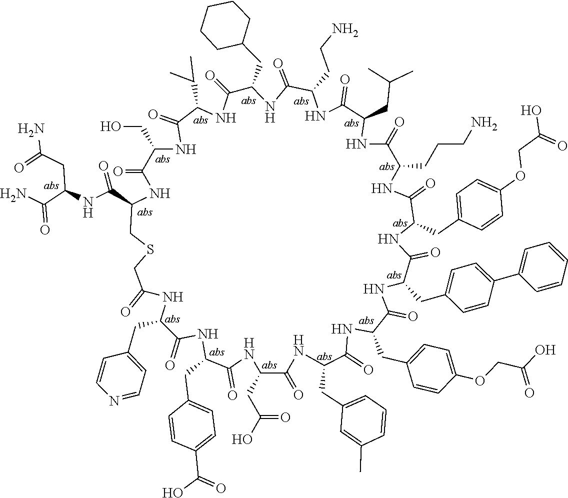 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2313 was prepared on a 50 μmol scale. The yield of the product was 3.4 mg, and its estimated purity by LCMS analysis was 92.3%. Analysis condition B: Retention time=1.68 min; ESI-MS(+) m/z [M+2H]²⁺: 1112.2.

### Preparation of Compound 2314

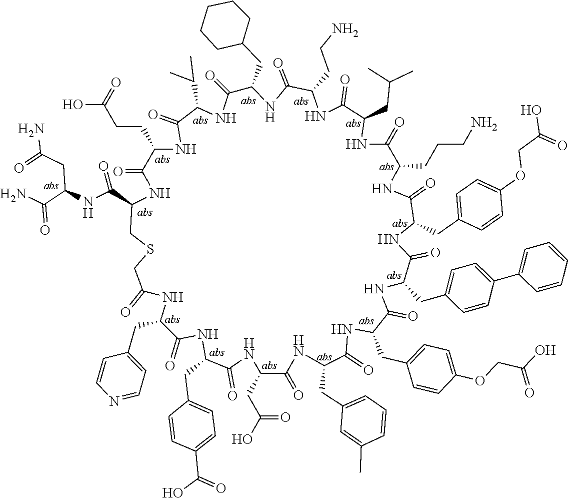 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2314 was prepared on a 50 μmol scale. The yield of the product was 4.6 mg, and its estimated purity by LCMS analysis was 92%. Analysis condition A: Retention time=1.32 min; ESI-MS(+) m/z [M+2H]²⁺: 1133.1.

### Preparation of Compound 2315

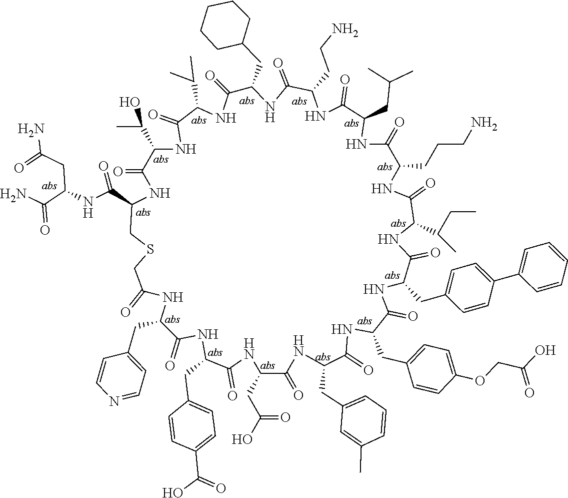 [SMILES: CCC(C)[C@@H]1NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(N)=O)NC(=O)[C@H]([C@@H](C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2315 was prepared on a 50 μmol scale. The yield of the product was 31.2 mg, and its estimated purity by LCMS analysis was 100%. Analysis condition A: Retention time=1.65 min; ESI-MS(+) m/z [M+2H]²⁺: 1065.

### Preparation of Compound 2316

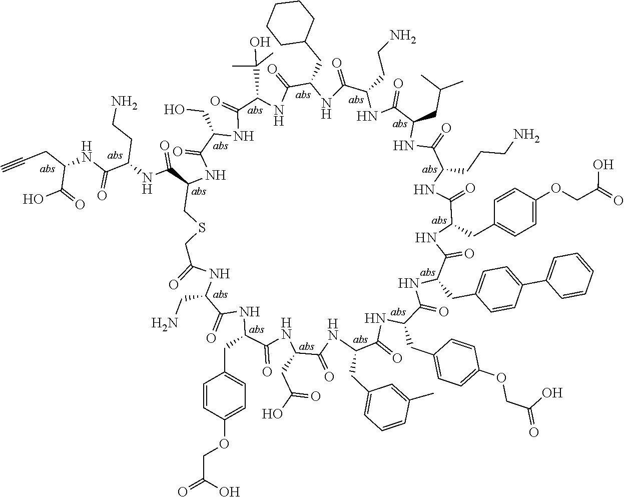 [SMILES: C#CC[C@H](NC(=O)[C@H](CCN)NC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2316 was prepared on a 50 μmol scale. The yield of the product was 1.1 mg, and its estimated purity by LCMS analysis was 97.1%. Analysis condition B: Retention time=1.53 min; ESI-MS(+) m/z [M+2H]²⁺: 1145.1.

### Preparation of Compound 2317

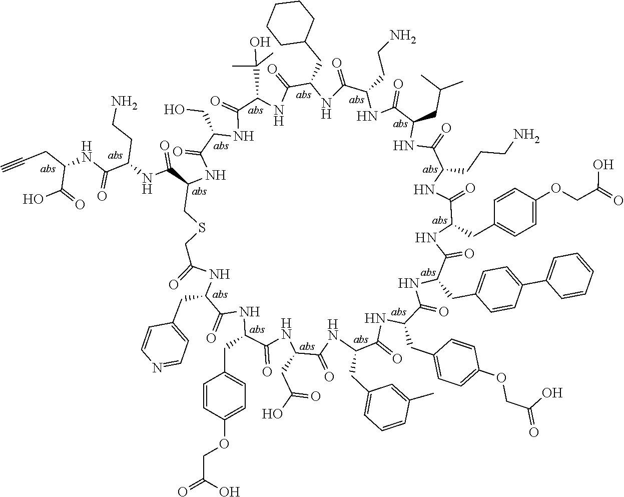 [SMILES: C#CC[C@H](NC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2317 was prepared on a 50 μmol scale. The yield of the product was 7.4 mg, and its estimated purity by LCMS analysis was 90.6%. Analysis condition B: Retention time=1.52 min; ESI-MS(+) m/z [M+2H]²⁺: 1176.1.

### Preparation of Compound 2318

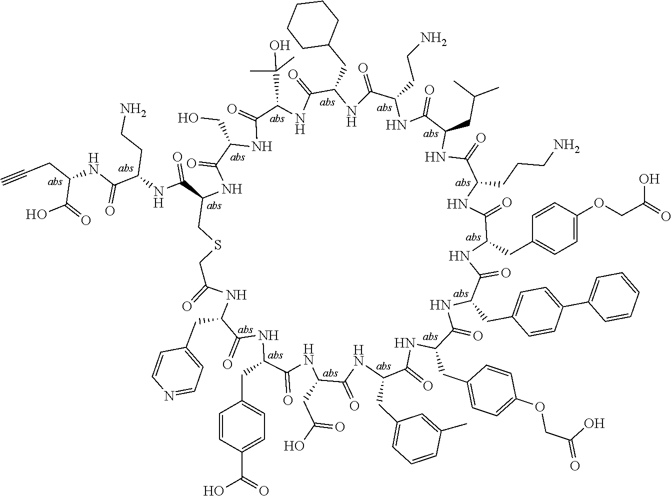 [SMILES: C#CC[C@H](NC(=O)[C@H](CCN)NC(=O)[C@@H]1CSCC(=O)N[C@@H](Cc2ccncc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2318 was prepared on a 50 μmol scale. The yield of the product was 1.7 mg, and its estimated purity by LCMS analysis was 100%. Analysis condition A: Retention time=1.34 min; ESI-MS(+) m/z [M+2H]²⁺: 1161.1.

### Preparation of Compound 2319

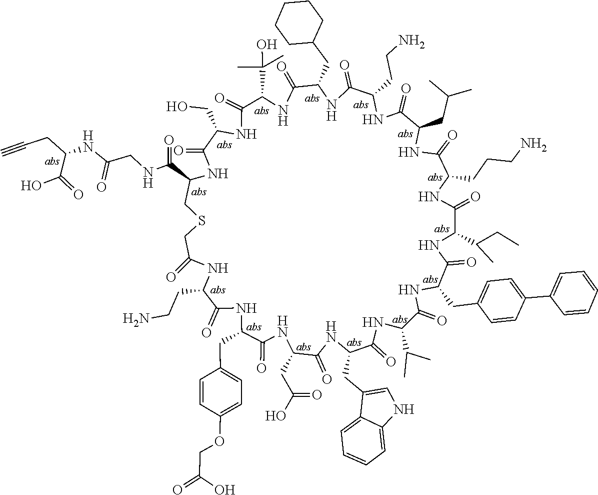 [SMILES: C#CC[C@H](NC(=O)CNC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)CC)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2319 was prepared on a 50 μmol scale. The yield of the product was 4.5 mg, and its estimated purity by LCMS analysis was 92.3%. Analysis condition A: Retention time=1.62 min; ESI-MS(+) m/z [M+2H]²⁺: 1028.

### Preparation of Compound 2320

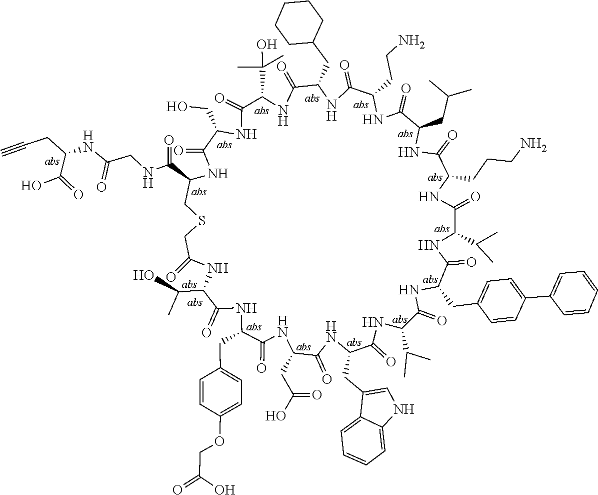 [SMILES: C#CC[C@H](NC(=O)CNC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2320 was prepared on a 50 μmol scale. The yield of the product was 4.8 mg, and its estimated purity by LCMS analysis was 97.1%. Analysis condition B: Retention time=1.65 min; ESI-MS(+) m/z [M+2H]²⁺: 1021.1.

### Preparation of Compound 2321

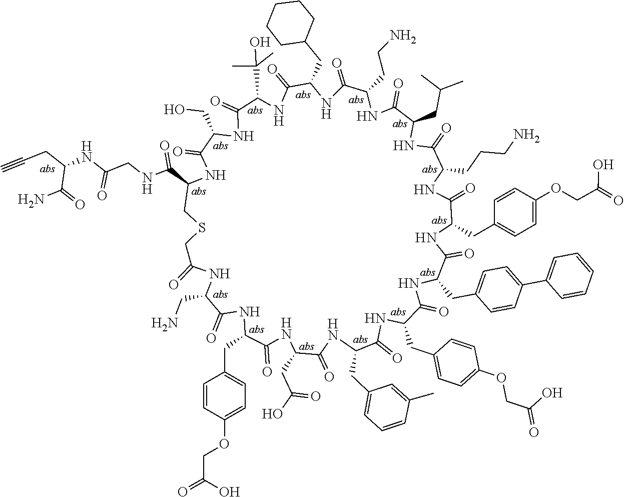 [SMILES: C#CC[C@H](NC(=O)CNC(=O)[C@@H]1CSCC(=O)N[C@@H](CN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2321 was prepared on a 50 μmol scale. The yield of the product was 19.9 mg, and its estimated purity by LCMS analysis was 91.9%. Analysis condition A: Retention time=1.54 min; ESI-MS(+) m/z [M+2H]²⁺: 1123.2.

### Preparation of Compound 2322

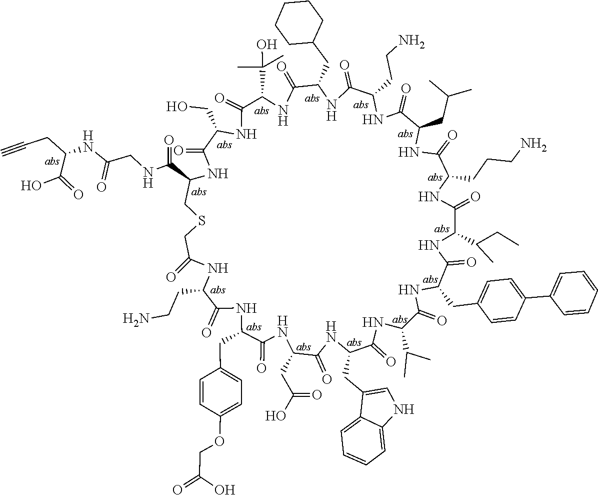 [SMILES: C#CC[C@H](NC(=O)CNC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)CC)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2322 was prepared on a 50 μmol scale. The yield of the product was 16.7 mg, and its estimated purity by LCMS analysis was 96.1%. Analysis condition A: Retention time=1.61 min; ESI-MS(+) m/z [M+2H]²⁺: 1027.5.

### Preparation of Compound 2323

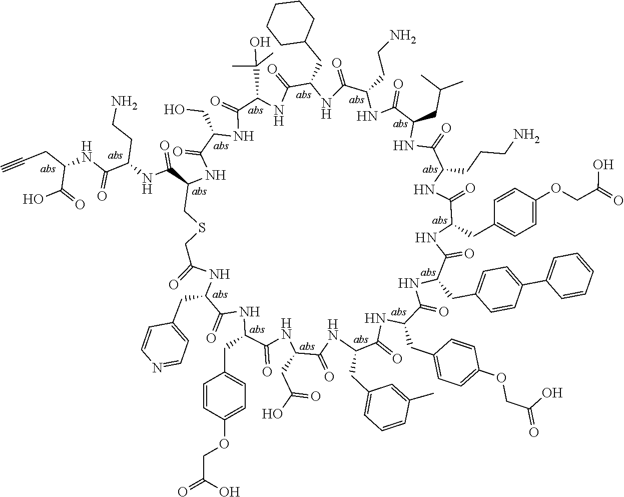 [SMILES: C#CC[C@H](NC(=O)[C@H](CCN)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2323 was prepared on a 50 μmol scale. The yield of the product was 9.7 mg, and its estimated purity by LCMS analysis was 96%. Analysis condition A: Retention time=1.35 min; ESI-MS(+) m/z [M+2H]²⁺: 1154.2.

### Preparation of Compound 2324

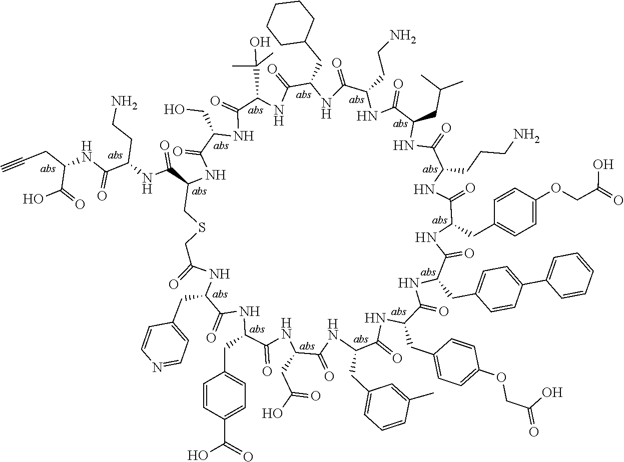 [SMILES: C#CC[C@H](NC(=O)[C@H](CCN)NC(=O)[C@@H]1CSCC(=O)N[C@@H](Cc2ccncc2)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2324 was prepared on a 50 μmol scale. The yield of the product was 10.7 mg, and its estimated purity by LCMS analysis was 96.9%. Analysis condition B: Retention time=1.53 min; ESI-MS(+) m/z [M+2H]²⁺: 1160.1.

### Preparation of Compound 2325

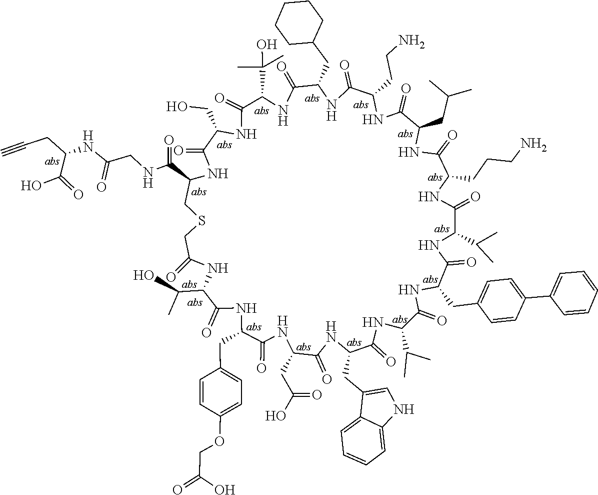 [SMILES: C#CC[C@H](NC(=O)CNC(=O)[C@@H]1CSCC(=O)N[C@@H]([C@@H](C)O)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2325 was prepared on a 50 μmol scale. The yield of the product was 19.5 mg, and its estimated purity by LCMS analysis was 94.8%. Analysis condition A: Retention time=1.59 min; ESI-MS(+) m/z [M+2H]²⁺: 1020.2.

### Preparation of Compound 2326

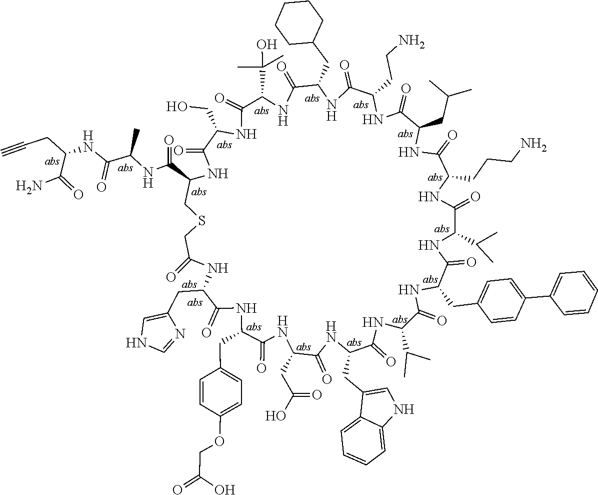 [SMILES: C#CC[C@H](NC(=O)[C@@H](C)NC(=O)[C@@H]1CSCC(=O)N[C@@H](Cc2c[nH]cn2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2326 was prepared on a 50 μmol scale. The yield of the product was 25.4 mg, and its estimated purity by LCMS analysis was 97.3%. Analysis condition A: Retention time=1.69 min; ESI-MS(+) m/z [M+2H]²⁺: 1046.2.

### Preparation of Compound 2327

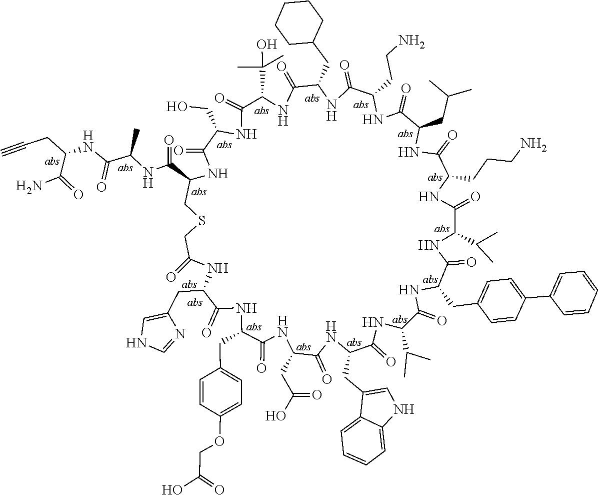 [SMILES: C#CC[C@H](NC(=O)[C@@H](C)NC(=O)[C@@H]1CSCC(=O)N[C@@H](Cc2c[nH]cn2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2327 was prepared on a 50 μmol scale. The yield of the product was 9.9 mg, and its estimated purity by LCMS analysis was 92.5%. Analysis condition B: Retention time=1.61 min; ESI-MS(+) m/z [M+3H]³⁺: 698.1.

### Preparation of Compound 2328

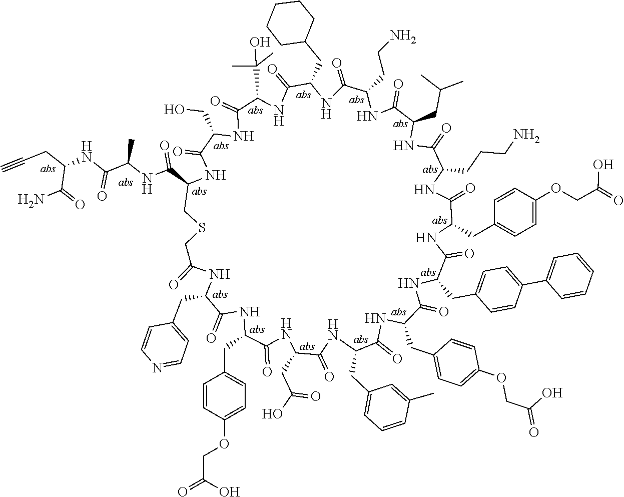 [SMILES: C#CC[C@H](NC(=O)[C@@H](C)NC(=O)[C@@H]1CSCC(=O)N[C@@H](Cc2ccncc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2328 was prepared on a 50 μmol scale. The yield of the product was 13.7 mg, and its estimated purity by LCMS analysis was 95.5%. Analysis condition B: Retention time=1.59 min; ESI-MS(+) m/z [M+2H]²⁺: 1161.3.

### Preparation of Compound 2329

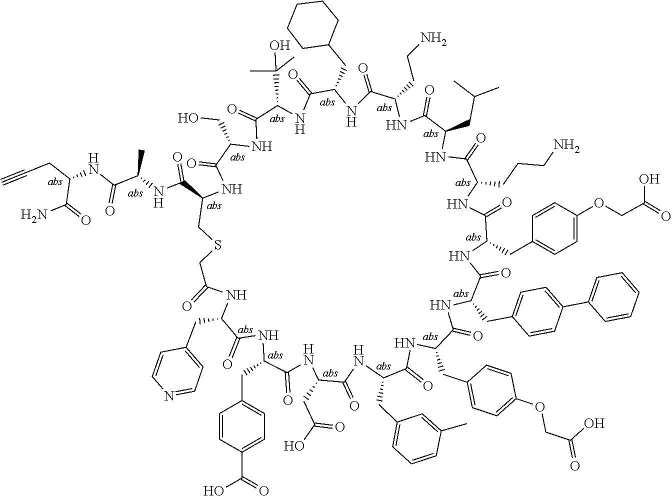

Compound 2329 was prepared on a 50 μmol scale. The yield of the product was 15.1 mg, and its estimated purity by LCMS analysis was 95.9%. Analysis condition B: Retention time=1.66 min; ESI-MS(+) m/z [M+2H]²⁺: 1146.2.

### Preparation of Compound 2330

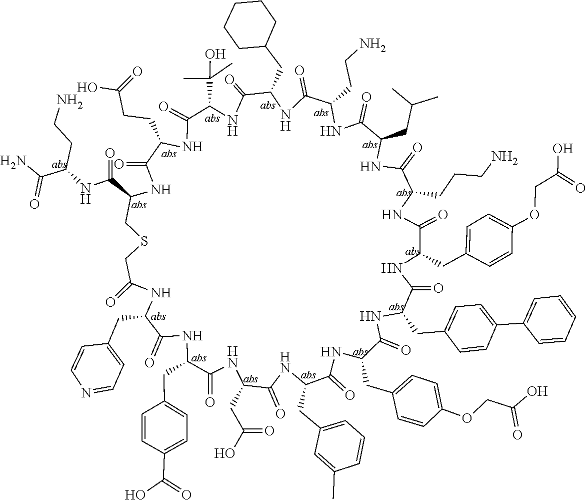 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2330 was prepared on a 50 μmol scale. The yield of the product was 19.7 mg, and its estimated purity by LCMS analysis was 93.5%. Analysis condition B: Retention time=1.49 min; ESI-MS(+) m/z [M+2H]²⁺: 1134.

### Preparation of Compound 2500

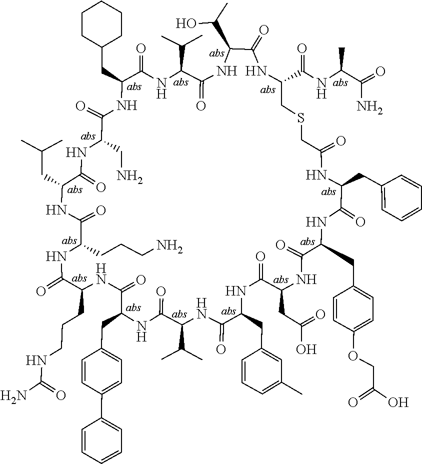 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccccc3)NC(=O)CSC[C@@H](C(=O)N[C@@H](C)C(N)=O)NC(=O)[C@H](C(C)O)NC(=O)[C@H](C(C)C)NC(=O)[C@H](CC3CCCCC3)NC(=O)[C@H](CN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2500 was prepared on a 50 μmol scale. The yield of the product was 18.9 mg, and its estimated purity by LCMS analysis was 95.1%. Analysis condition B: Retention time=1.82 min; ESI-MS(+) m/z [M+2H]²⁺: 1012.2.

### Preparation of Compound 2501

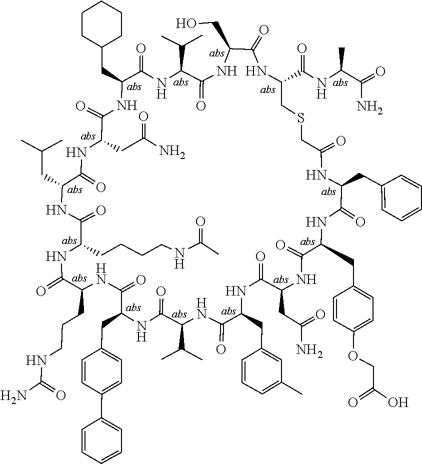 [SMILES: CC(=O)NCCCC[C@@H]1NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(N)=O)NC(=O)[C@H](Cc2ccc(OCC(=O)O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC1=O]

Compound 2501 was prepared on a 50 μmol scale. The yield of the product was 17.5 mg, and its estimated purity by LCMS analysis was 100%. Analysis condition A: Retention time=1.6 min; ESI-MS(+) m/z [M+2H]²⁺: 1047.2.

### Preparation of Compound 2502

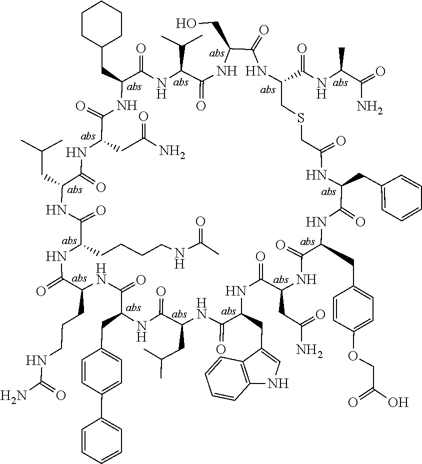 [SMILES: CC(=O)NCCCC[C@@H]1NC(=O)[C@H](CCCNC(N)=O)NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(N)=O)NC(=O)[C@H](Cc2ccc(OCC(=O)O)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CC(N)=O)NC(=O)[C@@H](CC(C)C)NC1=O]

Compound 2502 was prepared on a 50 μmol scale. The yield of the product was 48.6 mg, and its estimated purity by LCMS analysis was 95.2%. Analysis condition A: Retention time=1.63 min; ESI-MS(+) m/z [M+2H]²⁺: 1066.2.

### Preparation of Compound 2503

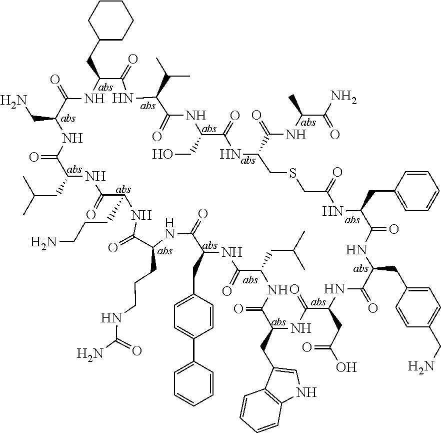 [SMILES: CC(C)C[C@@H]1NC(=O)[C@H](Cc2c[nH]c3ccccc23)NC(=O)[C@H](CC(=O)O)NC(=O)[C@H](Cc2ccc(CN)cc2)NC(=O)[C@H](Cc2ccccc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC1=O]

Compound 2503 was prepared on a 50 μmol scale. The yield of the product was 11 mg, and its estimated purity by LCMS analysis was 100%. Analysis condition A: Retention time=1.82 min; ESI-MS(+) m/z [M+2H]²⁺: 1002.

### Preparation of Compound 2504

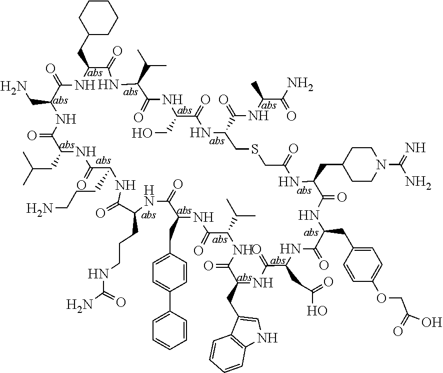 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC2CCN(C(=N)N)CC2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CN)NC1=O]

Compound 2504 was prepared on a 50 μmol scale. The yield of the product was

9.7 mg, and its estimated purity by LCMS analysis was 99.2%. Analysis condition A: Retention time=1.62 min; ESI-MS(+) m/z [M+2H]²⁺: 1042.2.

### Preparation of Compound 2505

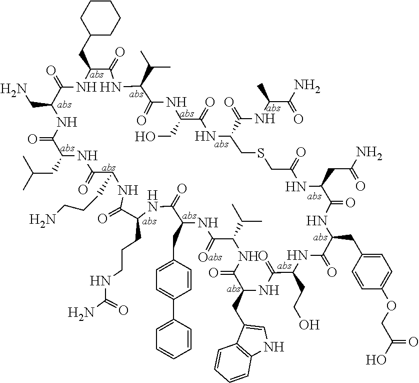 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CCO)NC(=O)[C@H](Cc2ccc(OCC(=O)O)cc2)NC(=O)[C@H](CC(N)=O)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CN)NC1=O]

Compound 2505 was prepared on a 50 μmol scale. The yield of the product was 8.8 mg, and its estimated purity by LCMS analysis was 97.6%. Analysis condition A: Retention time=1.66 min; ESI-MS(+) m/z [M+2H]²⁺: 1008.2.

### Preparation of Compound 2506

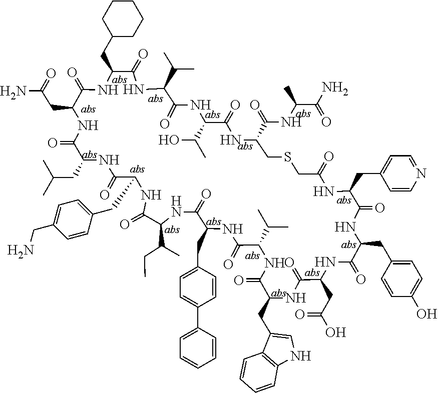 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](Cc2ccncc2)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CC(N)=O)NC(=O)[C@@H](CC(C)C)NC(=O)[C@H](Cc2ccc(CN)cc2)NC1=O]

Compound 2506 was prepared on a 50 μmol scale. The yield of the product was 46 mg, and its estimated purity by LCMS analysis was 91.4%. Analysis condition A: Retention time=1.81 min; ESI-MS(+) m/z [M+2H]²⁺: 1019.

### Preparation of Compound 2507

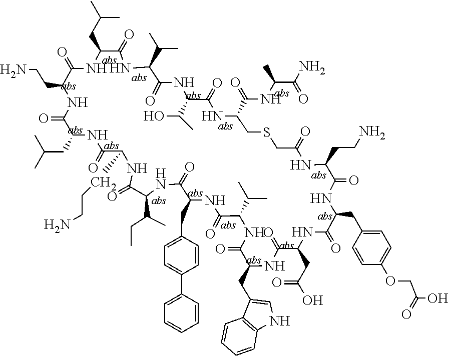 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](C)C(N)=O)NC(=O)[C@H](C(C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2507 was prepared on a 25 μmol scale. The yield of the product was 6.7 mg, and its estimated purity by LCMS analysis was 98.6%. Analysis condition B: Retention time=1.48 min; ESI-MS(+) m/z [M+H]⁺: 1930.1.

### Preparation of Compound 2508

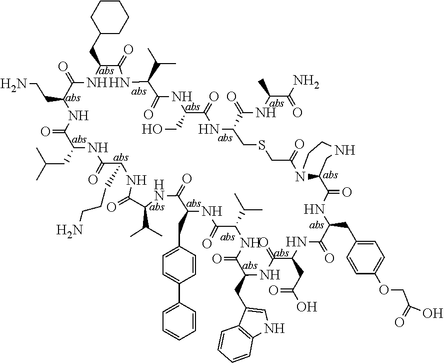

Compound 2508 was prepared on a 50 μmol scale. The yield of the product was 16.2 mg, and its estimated purity by LCMS analysis was 100%. Analysis condition A: Retention time=1.77 min; ESI-MS(+) m/z [M+H]⁺: 1967.8.

### Preparation of Compound 2509

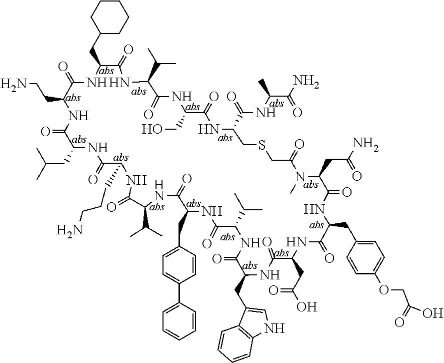 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC(N)=O)N(C)C(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2509 was prepared on a 50 μmol scale. The yield of the product was 18 mg, and its estimated purity by LCMS analysis was 95.3%. Analysis condition A: Retention time=1.65 min; ESI-MS(+) m/z [M+H]⁺: 1984.

### Preparation of Compound 2510

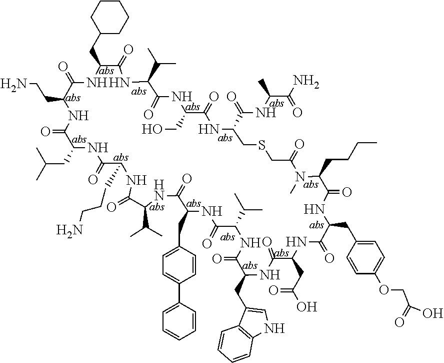 [SMILES: CCCC[C@H]1C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N[C@H](C(=O)N[C@@H](C)C(N)=O)CSCC(=O)N1C]

Compound 2510 was prepared on a 50 μmol scale. The yield of the product was 8.7 mg, and its estimated purity by LCMS analysis was 100%. Analysis condition A: Retention time=1.94 min; ESI-MS(+) m/z [M+H]⁺: 1983.1.

### Preparation of Compound 2511

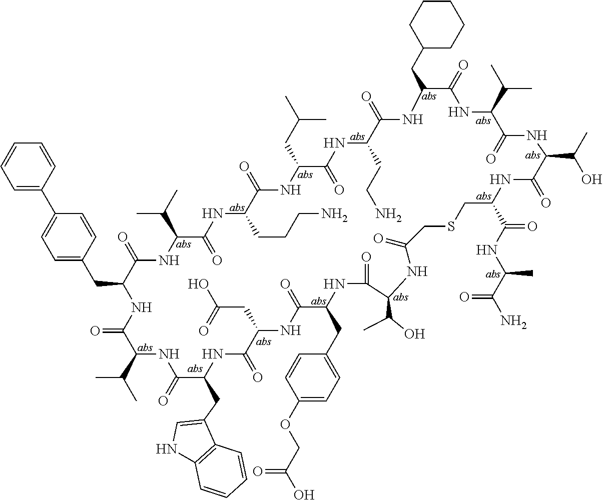

Compound 2511 was prepared on a 50 μmol scale. The yield of the product was 14.9 mg, and its estimated purity by LCMS analysis was 100%. Analysis condition A: Retention time=1.67, 1.72 min; ESI-MS(+) m/z [M+H]⁺: 1957.05, 1957.24.

### Preparation of Compound 2512

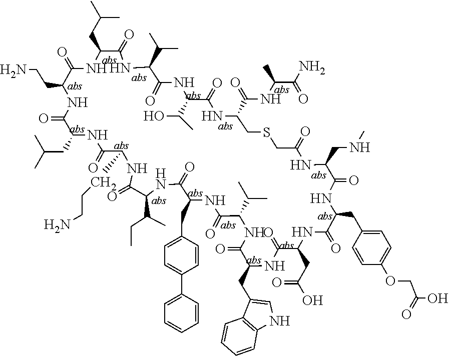 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CNC)NC(=O)CSC[C@@H](C(=O)N[C@@H](C)C(N)=O)NC(=O)[C@H](C(C)O)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2512 was prepared on a 25 μmol scale. The yield of the product was 7 mg, and its estimated purity by LCMS analysis was 97.7%. Analysis condition A: Retention time=1.66 min; ESI-MS(+) m/z [M+H]⁺: 1930.

### Preparation of Compound 2513

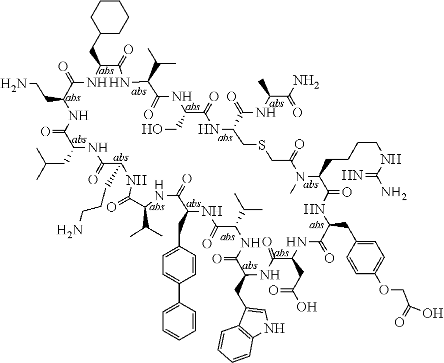 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)N(C)C(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2513 was prepared on a 50 μmol scale. The yield of the product was 6.1 mg, and its estimated purity by LCMS analysis was 99%. Analysis condition A: Retention time=1.65 min; ESI-MS(+) m/z [M+2H]²⁺: 1014.1.

### Preparation of Compound 2514

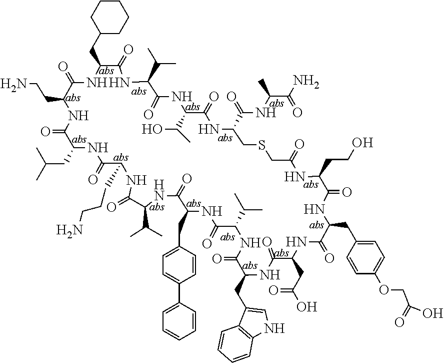 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCO)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2514 was prepared on a 50 μmol scale. The yield of the product was 11.7 mg, and its estimated purity by LCMS analysis was 96%. Analysis condition B: Retention time=1.53 min; ESI-MS(+) m/z [M+H]⁺: 1958.

### Preparation of Compound 2515

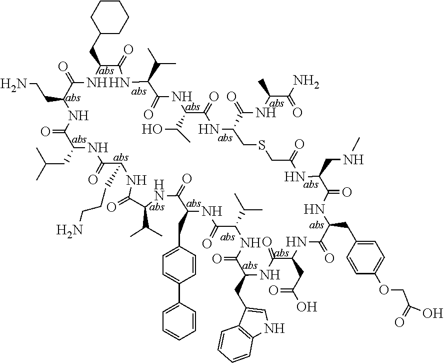 [SMILES: CNC[C@@H]1NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC1=O]

Compound 2515 was prepared on a 50 μmol scale. The yield of the product was 2.3 mg, and its estimated purity by LCMS analysis was 95.1%. Analysis condition A: Retention time=1.78 min; ESI-MS(+) m/z [M+H]⁺: 1956.1.

### Preparation of Compound 2516

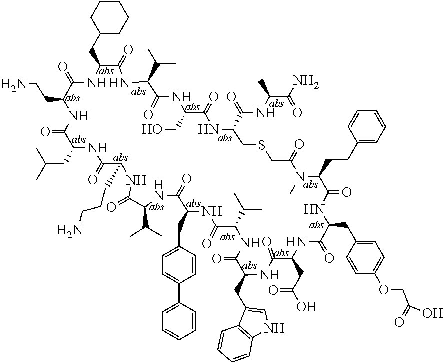 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCc2ccccc2)N(C)C(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2516 was prepared on a 50 μmol scale. The yield of the product was 13.9 mg, and its estimated purity by LCMS analysis was 97.2%. Analysis condition B: Retention time=1.85 min; ESI-MS(+) m/z [M+2H]²⁺: 1009.3.

### Preparation of Compound 2517

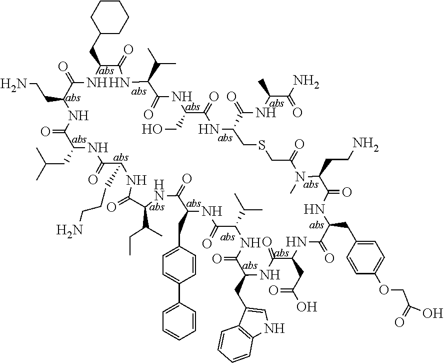 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)N(C)C(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2517 was prepared on a 50 μmol scale. The yield of the product was 7.3 mg, and its estimated purity by LCMS analysis was 95.5%. Analysis condition B: Retention time=1.6 min; ESI-MS(+) m/z [M+H]⁺: 1971.3.

### Preparation of Compound 2518

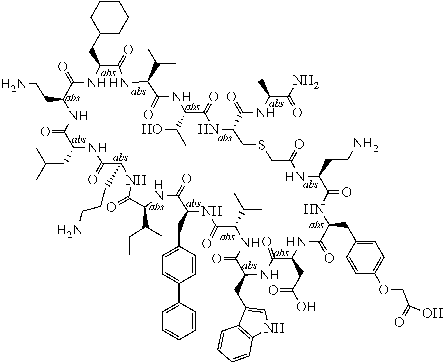 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2518 was prepared on a 50 μmol scale. The yield of the product was 13.1 mg, and its estimated purity by LCMS analysis was 98.6%. Analysis condition B: Retention time=1.73 min; ESI-MS(+) m/z [M+H]⁺: 1984.3.

### Preparation of Compound 2519

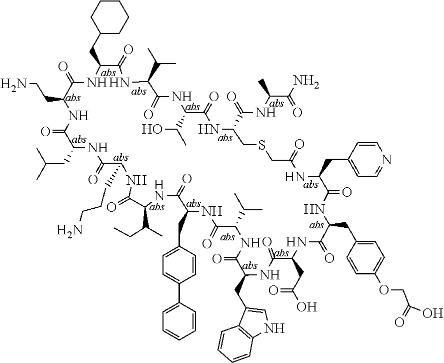 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2519 was prepared on a 50 μmol scale. The yield of the product was 5.2 mg, and its estimated purity by LCMS analysis was 98.6%. Analysis condition A: Retention time=1.72 min; ESI-MS(+) m/z [M+2H]²⁺: 1010.2.

### Preparation of Compound 2520

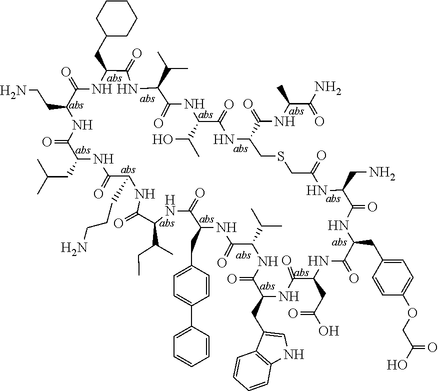 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2520 was prepared on a 50 μmol scale. The yield of the product was 6.9 mg, and its estimated purity by LCMS analysis was 100%. Analysis condition A: Retention time=1.78 min; ESI-MS(+) m/z [M+H]⁺: 1957.

### Preparation of Compound 2521

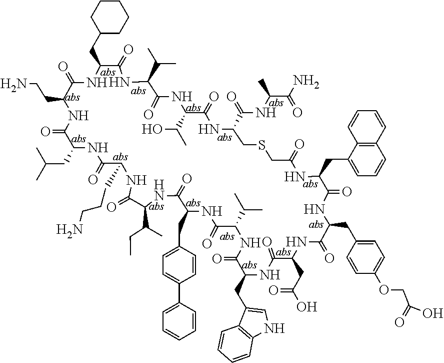 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cccc3ccccc23)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2521 was prepared on a 50 μmol scale. The yield of the product was 16.2 mg, and its estimated purity by LCMS analysis was 95%. Analysis condition B: Retention time=1.86 min; ESI-MS(+) m/z [M+2H]²⁺: 1034.2.

### Preparation of Compound 2522

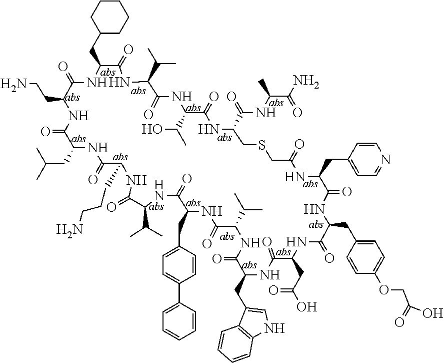 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2522 was prepared on a 50 μmol scale. The yield of the product was 7.7 mg, and its estimated purity by LCMS analysis was 100%. Analysis condition A: Retention time=1.68 min; ESI-MS(+) m/z [M+2H]²⁺: 1003.

### Preparation of Compound 2523

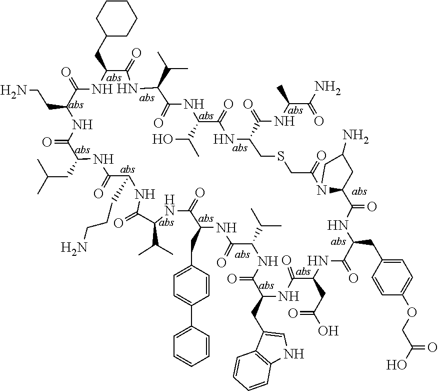

Compound 2523 was prepared on a 50 μmol scale. The yield of the product was 11.1 mg, and its estimated purity by LCMS analysis was 95%. Analysis condition B: Retention time=1.54 min; ESI-MS(+) m/z [M+H]⁺: 1968.3.

### Preparation of Compound 2524

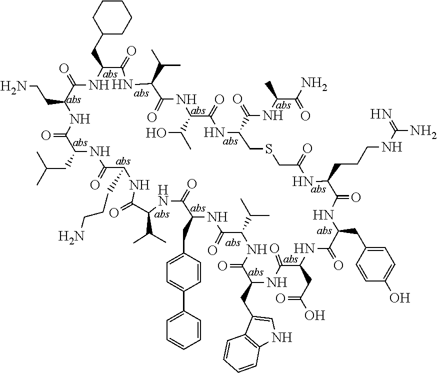 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCCNC(=N)N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2524 was prepared on a 50 μmol scale. The yield of the product was 6.3 mg, and its estimated purity by LCMS analysis was 95.1%. Analysis condition B: Retention time=1.61 min; ESI-MS(+) m/z [M+2H]²⁺: 978.1.

### Preparation of Compound 2525

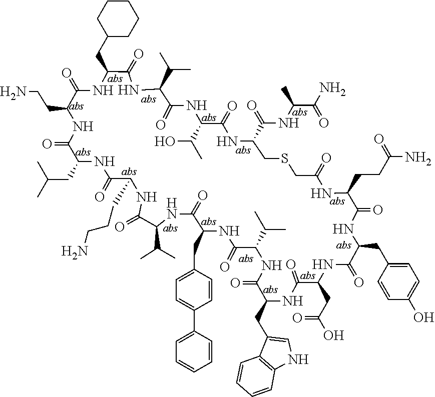 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CCC(N)=O)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2525 was prepared on a 50 μmol scale. The yield of the product was 5.8 mg, and its estimated purity by LCMS analysis was 100%. Analysis condition A: Retention time=1.73 min; ESI-MS(+) m/z [M+H]⁺: 1925.9.

### Preparation of Compound 2526

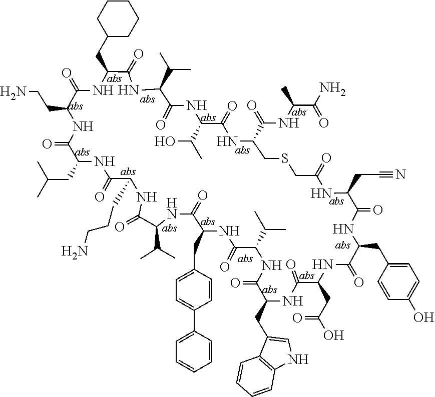 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC#N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2526 was prepared on a 50 μmol scale. The yield of the product was 3.3 mg, and its estimated purity by LCMS analysis was 95%. Analysis condition A: Retention time=1.7 min; ESI-MS(+) m/z [M+H]⁺: 1894.1.

### Preparation of Compound 2527

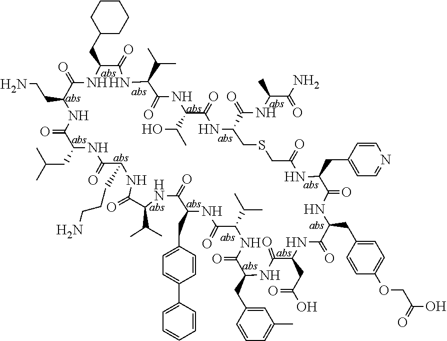 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](C)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2527 was prepared on a 50 μmol scale. The yield of the product was 2.4 mg, and its estimated purity by LCMS analysis was 95.2%. Analysis condition B: Retention time=1.52 min; ESI-MS(+) m/z [M+2H]²⁺: 983.8.

### Preparation of Compound 2528

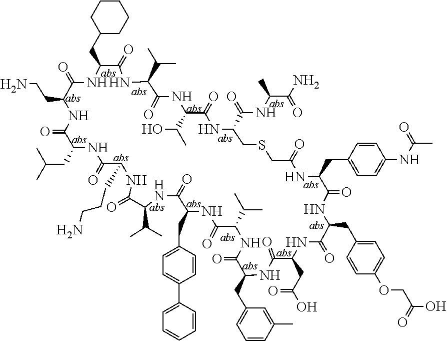 [SMILES: CC(=O)Nc1ccc(C[C@@H]2NC(=O)CSC[C@@H](C(=O)N[C@@H](C)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC(=O)[C@H](Cc3cccc(C)c3)NC(=O)[C@H](CC(=O)O)NC(=O)[C@H](Cc3ccc(OCC(=O)O)cc3)NC2=O)cc1]

Compound 2528 was prepared on a 50 μmol scale. The yield of the product was 1.1 mg, and its estimated purity by LCMS analysis was 100%. Analysis condition B: Retention time=1.63 min; ESI-MS(+) m/z [M+2H]²⁺: 1011.6.

### Preparation of Compound 2529

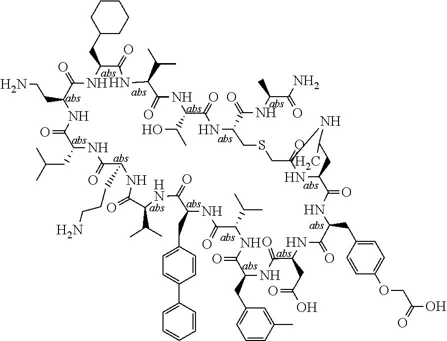

Compound 2529 was prepared on a 50 μmol scale. The yield of the product was 1.5 mg, and its estimated purity by LCMS analysis was 100%. Analysis condition B: Retention time=1.51 min; ESI-MS(+) m/z [M+2H]²⁺: 965.1.

### Preparation of Compound 2530

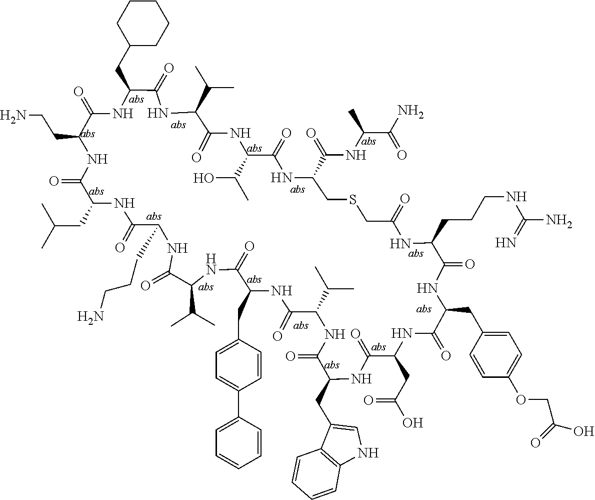 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(=N)N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2530 was prepared on a 50 μmol scale. The yield of the product was 3 mg, and its estimated purity by LCMS analysis was 95%. Analysis condition B: Retention time=1.53 min; ESI-MS(+) m/z [M+2H]²⁺: 1007.2.

### Preparation of Compound 2531

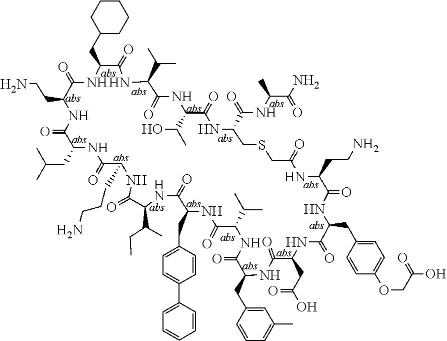 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2531 was prepared on a 25 μmol scale. The yield of the product was 1.3 mg, and its estimated purity by LCMS analysis was 100%. Analysis condition A: Retention time=1.66 min; ESI-MS(+) m/z [M+H]⁺: 1931.1.

### Preparation of Compound 2532

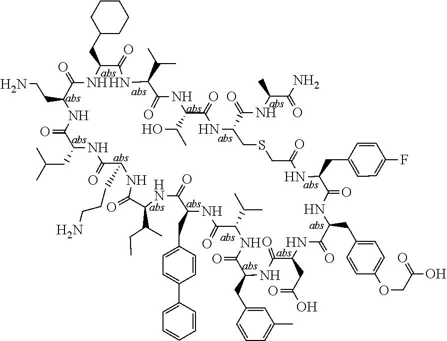 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(F)cc2)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2532 was prepared on a 25 μmol scale. The yield of the product was 1.3 mg, and its estimated purity by LCMS analysis was 100%. Analysis condition A: Retention time=1.82, 1.91 min; ESI-MS(+) m/z [M+H]⁺: 1997.

### Preparation of Compound 2533

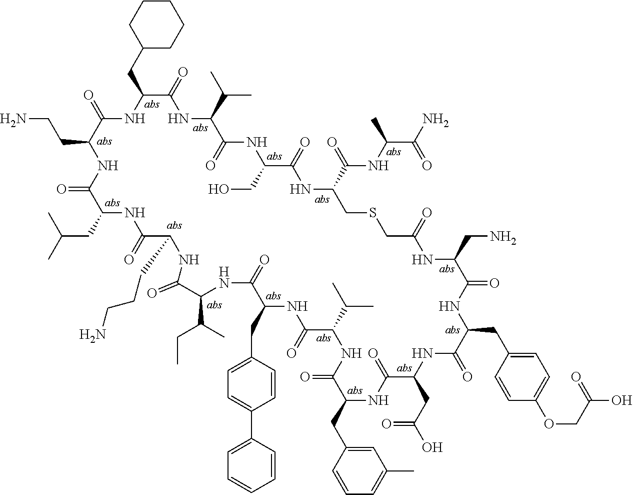 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2533 was prepared on a 25 μmol scale. The yield of the product was 1.1 mg, and its estimated purity by LCMS analysis was 100%. Analysis condition A: Retention time=1.67 min; ESI-MS(+) m/z [M+H]⁺: 1916.8.

### Preparation of Compound 2534

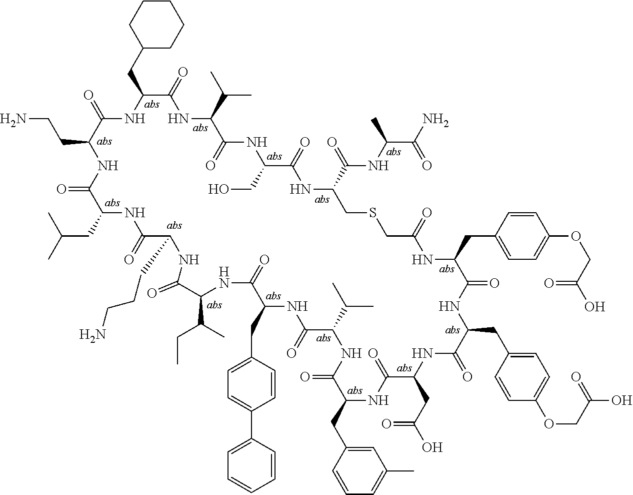 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2534 was prepared on a 25 μmol scale. The yield of the product was 2.1 mg, and its estimated purity by LCMS analysis was 100%. Analysis condition B: Retention time=1.73 min; ESI-MS(+) m/z [M+2H]²⁺: 1027.

### Preparation of Compound 2535

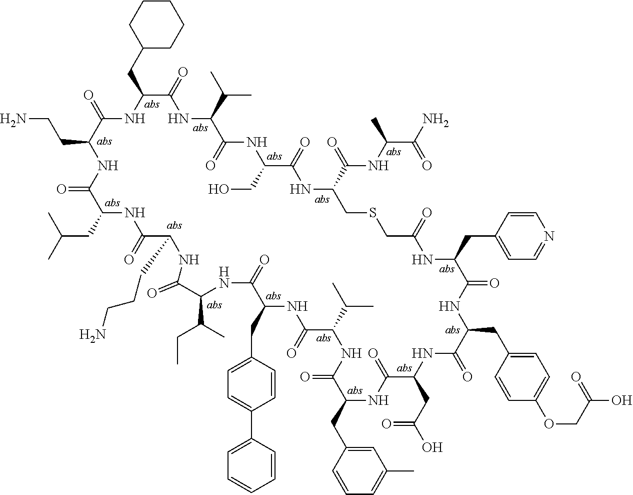 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2535 was prepared on a 25 μmol scale. The yield of the product was 1.3 mg, and its estimated purity by LCMS analysis was 100%. Analysis condition A: Retention time=1.75 min; ESI-MS(+) m/z [M+H]⁺: 1979.

### Preparation of Compound 2536

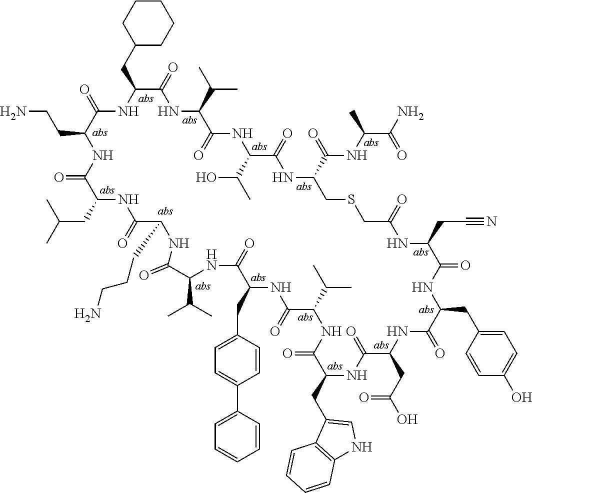 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](CC#N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2536 was prepared on a 50 μmol scale. The yield of the product was 4.9 mg, and its estimated purity by LCMS analysis was 96.8%. Analysis condition B: Retention time=1.73 min; ESI-MS(+) m/z [M+2H]²⁺: 948.1.

### Preparation of Compound 2537

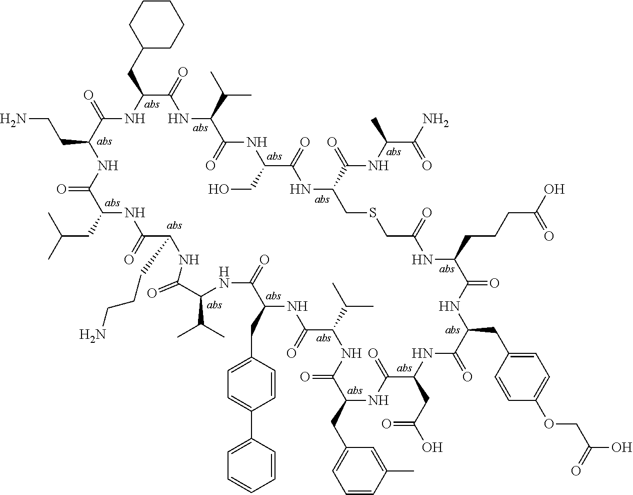 [SMILES: Cc1cccc(C[C@@H]2NC(=O)[C@H](CC(=O)O)NC(=O)[C@H](Cc3ccc(OCC(=O)O)cc3)NC(=O)[C@H](CCCC(=O)O)NC(=O)CSC[C@@H](C(=O)N[C@@H](C)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](C(C)C)NC(=O)[C@H](Cc3ccc(-c4ccccc4)cc3)NC(=O)[C@H](C(C)C)NC2=O)c1]

Compound 2537 was prepared on a 50 μmol scale. The yield of the product was 1.1 mg, and its estimated purity by LCMS analysis was 92.4%. Analysis condition B: Retention time=1.71 min; ESI-MS(+) m/z [M+2H]²⁺: 980.2.

### Preparation of Compound 2538

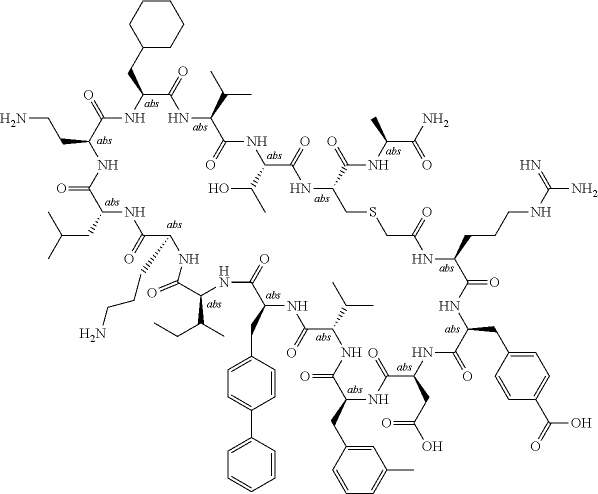 [SMILES: CCC(C)[C@@H]1NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(C(=O)O)cc2)NC(=O)[C@H](CCCNC(=N)N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC1=O]

Compound 2538 was prepared on a 50 μmol scale. The yield of the product was 6.8 mg, and its estimated purity by LCMS analysis was 98.1%. Analysis condition A: Retention time=1.72 min; ESI-MS(+) m/z [M+H]⁺: 1971.3.

### Preparation of Compound 2539

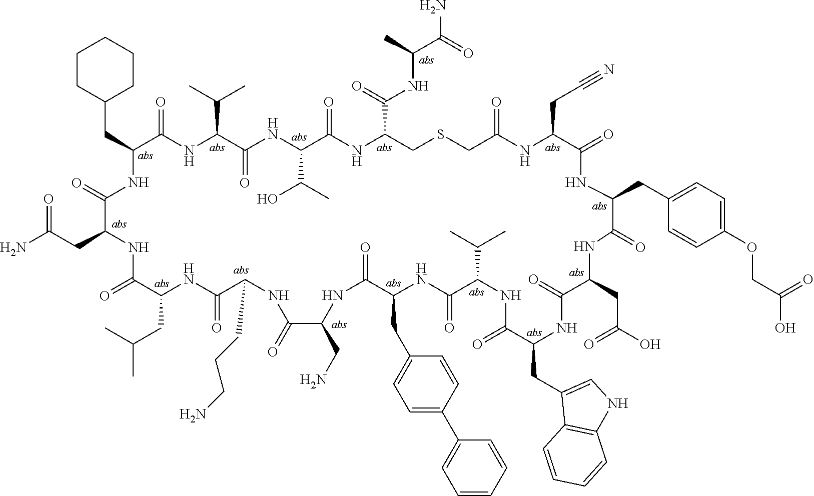 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CC#N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](CC(N)=O)NC1=O]

Compound 2539 was prepared on a 50 μmol scale. The yield of the product was 19.8 mg, and its estimated purity by LCMS analysis was 98.3%. Analysis condition B: Retention time=1.53 min; ESI-MS(+) m/z [M+2H]²⁺: 977.4.

### Preparation of Compound 2540

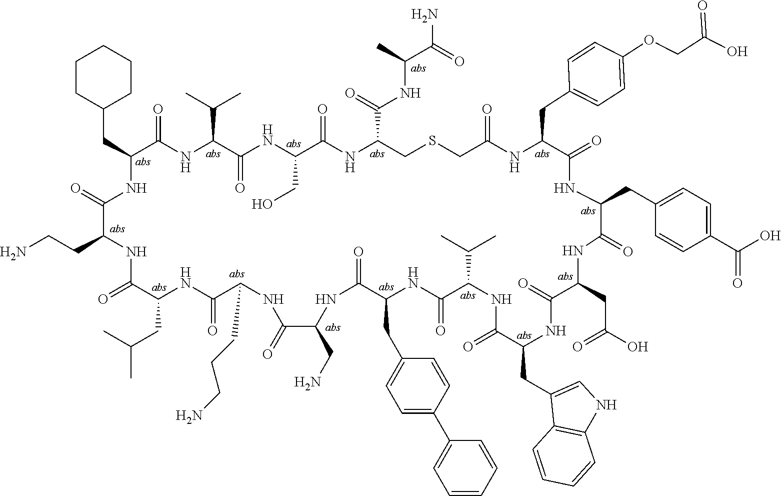 [SMILES: CC(C)C[C@H]1NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2540 was prepared on a 50 μmol scale. The yield of the product was 9.5 mg, and its estimated purity by LCMS analysis was 100%. Analysis condition A: Retention time=1.4 min; ESI-MS(+) m/z [M+2H]²⁺: 1011.

### Preparation of Compound 2541

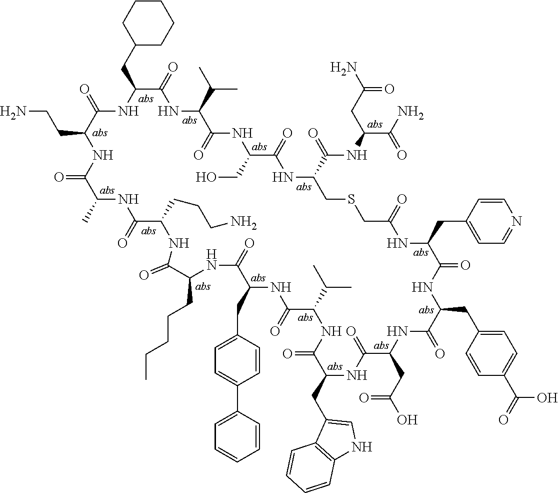 [SMILES: CCCCC[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](C)NC(=O)[C@H](CCCN)NC1=O]

Compound 2541 was prepared on a 50 μmol scale. The yield of the product was 7.7 mg, and its estimated purity by LCMS analysis was 100%. Analysis condition A: Retention time=1.73 min; ESI-MS(+) m/z [M+H]⁺: 1988.9.

### Preparation of Compound 2542

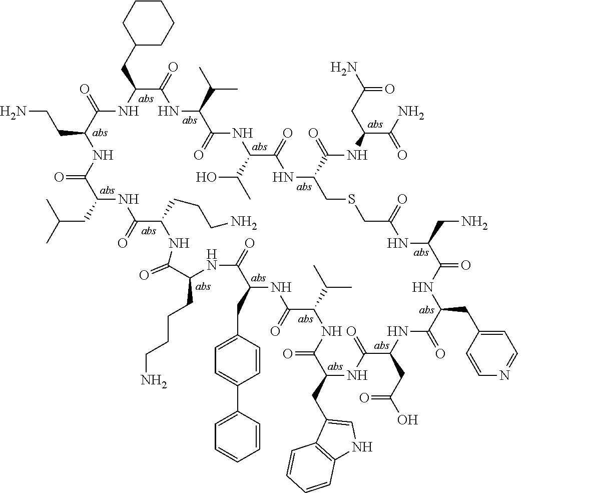 [SMILES: CC(C)C[C@H]1NC(=O)[C@H](CCCN)NC(=O)[C@H](CCCCN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2542 was prepared on a 50 μmol scale. The yield of the product was 20.1 mg, and its estimated purity by LCMS analysis was 100%. Analysis condition A: Retention time=1.57 min; ESI-MS(+) m/z [M+H]⁺: 1942.1.

### Preparation of Compound 2543

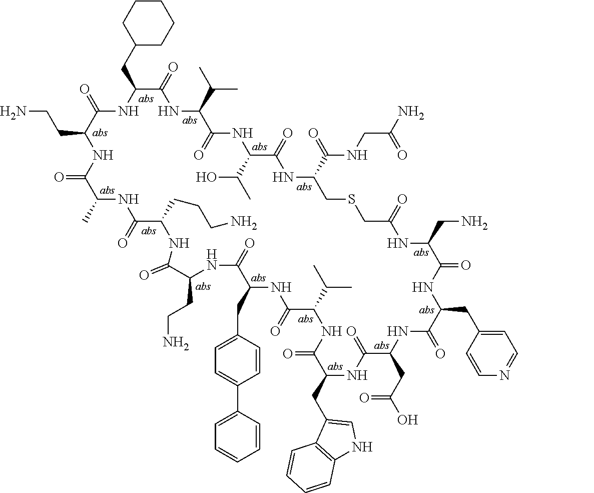 [SMILES: CC(C)[C@@H]1NC(=O)[C@H](Cc2c[nH]c3ccccc23)NC(=O)[C@H](CC(=O)O)NC(=O)[C@H](Cc2ccncc2)NC(=O)[C@H](CN)NC(=O)CSC[C@@H](C(=O)NCC(N)=O)NC(=O)[C@H](C(C)O)NC(=O)[C@H](C(C)C)NC(=O)[C@H](CC2CCCCC2)NC(=O)[C@H](CCN)NC(=O)[C@@H](C)NC(=O)[C@H](CCCN)NC(=O)[C@H](CCN)NC(=O)[C@H](Cc2ccc(-c3ccccc3)cc2)NC1=O]

Compound 2543 was prepared on a 50 μmol scale. The yield of the product was 22.8 mg, and its estimated purity by LCMS analysis was 95.7%. Analysis condition A: Retention time=1.34 min; ESI-MS(+) m/z [M+2H]²⁺: 908.

### Preparation of Compound 2544

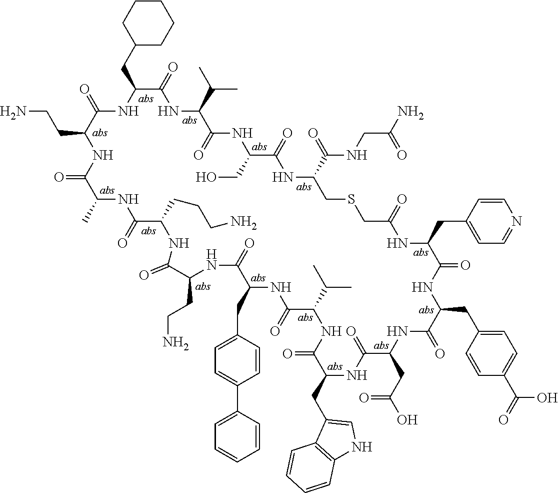 [SMILES: CC(C)[C@@H]1NC(=O)[C@H](Cc2c[nH]c3ccccc23)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)NCC(N)=O)NC(=O)[C@H](CO)NC(=O)[C@H](C(C)C)NC(=O)[C@H](CC2CCCCC2)NC(=O)[C@H](CCN)NC(=O)[C@@H](C)NC(=O)[C@H](CCCN)NC(=O)[C@H](CCN)NC(=O)[C@H](Cc2ccc(-c3ccccc3)cc2)NC1=O]

Compound 2544 was prepared on a 50 μmol scale. The yield of the product was 9 mg, and its estimated purity by LCMS analysis was 93.1%. Analysis condition B: Retention time=1.31 min; ESI-MS(+) m/z [M+H]⁺: 1904.8.

### Preparation of Compound 2545

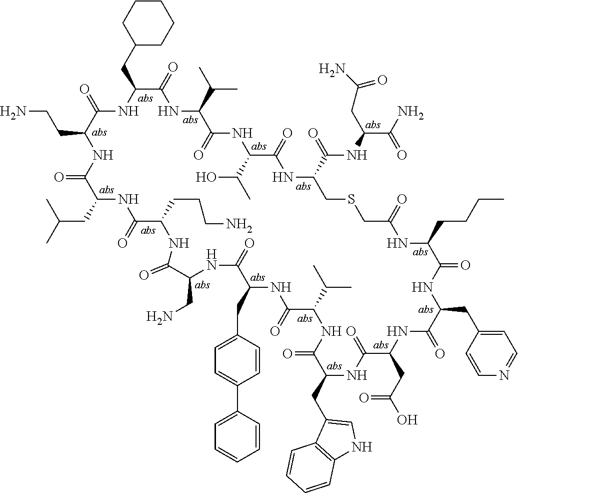 [SMILES: CCCC[C@@H]1NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CN)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccncc2)NC1=O]

Compound 2545 was prepared on a 50 μmol scale. The yield of the product was 14.3 mg, and its estimated purity by LCMS analysis was 96.1%. Analysis condition A: Retention time=1.52 min; ESI-MS(+) m/z [M+H]⁺: 1941.2.

### Preparation of Compound 2546

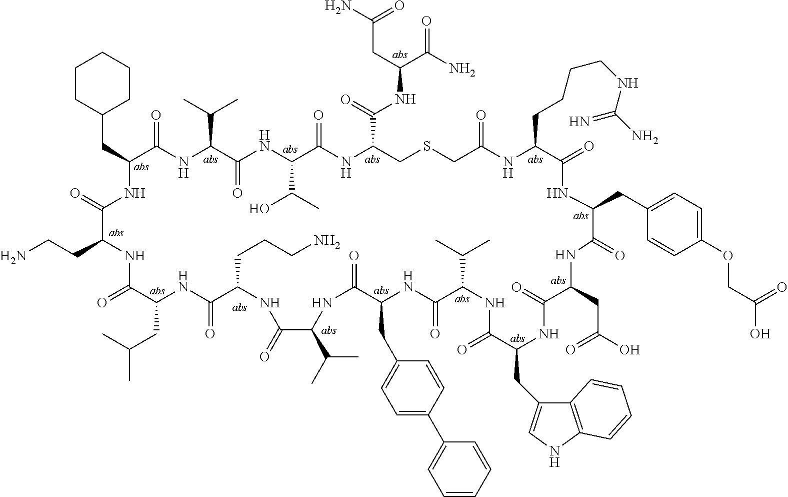 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2546 was prepared on a 50 μmol scale. The yield of the product was 2.3 mg, and its estimated purity by LCMS analysis was 100%. Analysis condition A: Retention time=1.64 min; ESI-MS(+) m/z [M+2H]²⁺: 1034.9.

### Preparation of Compound 2547

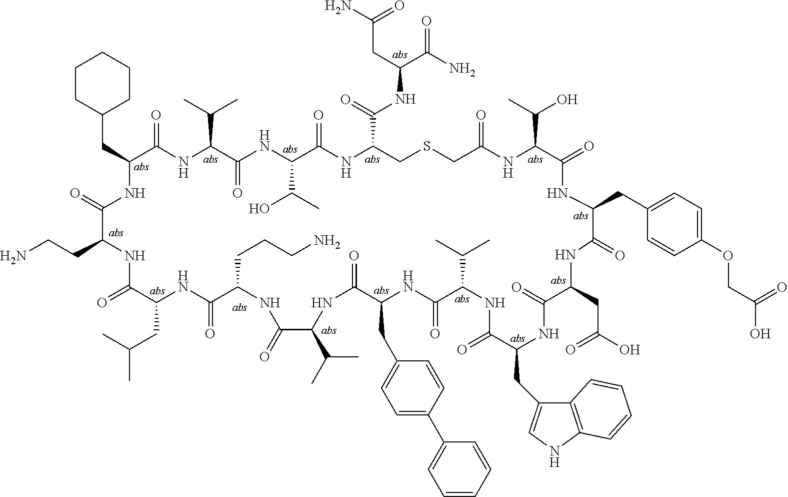 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2547 was prepared on a 50 μmol scale. The yield of the product was 1.4 mg, and its estimated purity by LCMS analysis was 100%. Analysis condition A: Retention time=1.6 min; ESI-MS(+) m/z [M+H]⁺: 1998.3.

### Preparation of Compound 2548

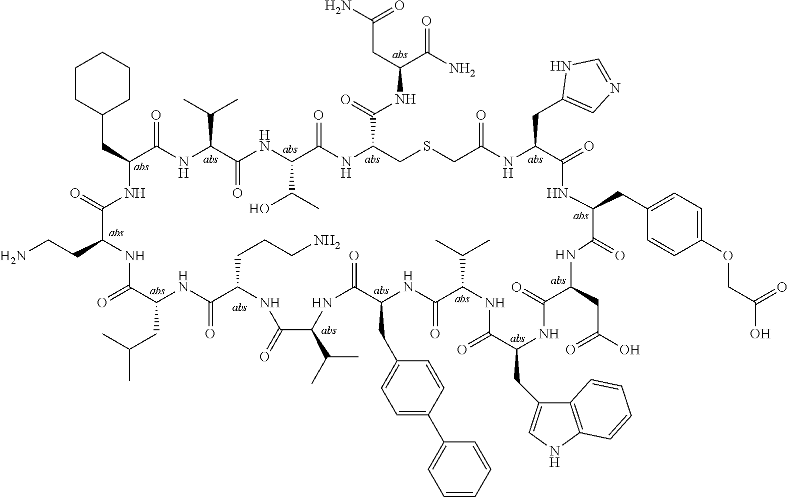 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2548 was prepared on a 50 μmol scale. The yield of the product was 8 mg, and its estimated purity by LCMS analysis was 97.4%. Analysis condition A: Retention time=1.68 min; ESI-MS(+) m/z [M+2H]²⁺: 1018.4.

### Preparation of Compound 2549

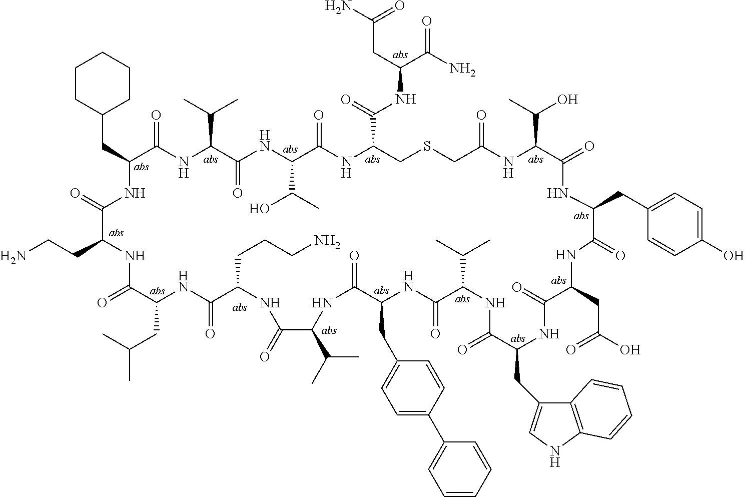 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(O)cc2)NC(=O)[C@H](C(C)O)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2549 was prepared on a 50 μmol scale. The yield of the product was 9.1 mg, and its estimated purity by LCMS analysis was 95.9%. Analysis condition A: Retention time=2.28 min; ESI-MS(+) m/z [M+2H]²⁺: 970.9.

### Preparation of Compound 2550

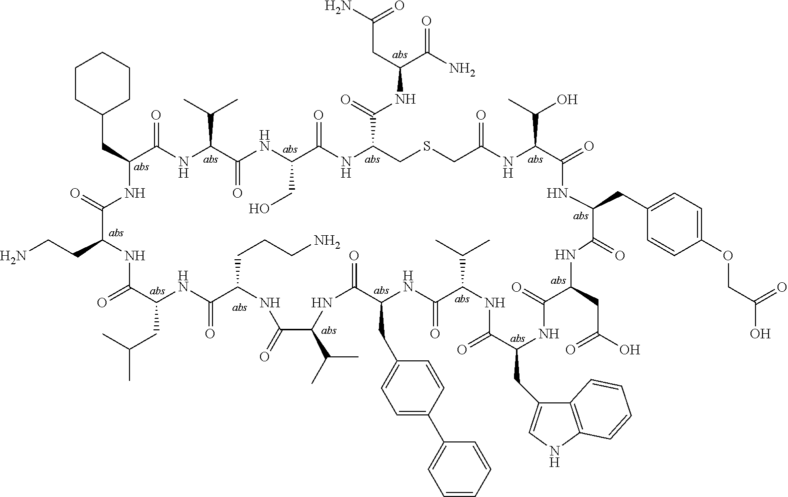 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2550 was prepared on a 50 μmol scale. The yield of the product was 3.6 mg, and its estimated purity by LCMS analysis was 98.1%. Analysis condition A: Retention time=1.58 min; ESI-MS(+) m/z [M+H]⁺: 1985.1.

### Preparation of Compound 2551

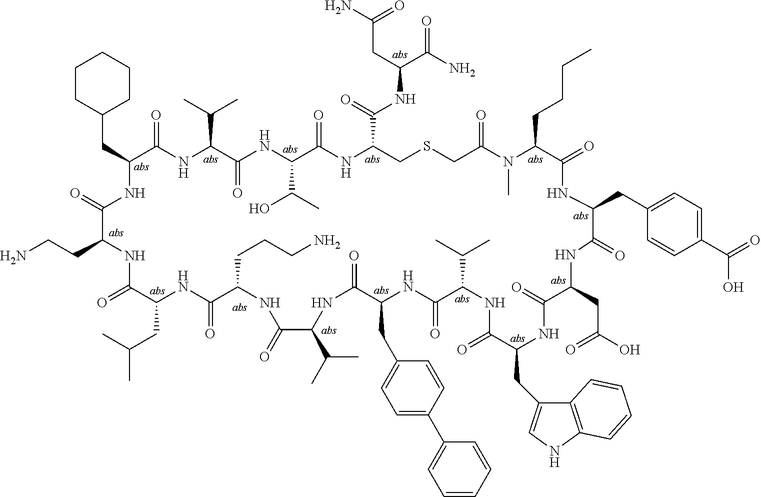 [SMILES: CCCC[C@H]1C(=O)N[C@@H](Cc2ccc(C(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](C(C)O)C(=O)N[C@H](C(=O)N[C@@H](CC(N)=O)C(N)=O)CSCC(=O)N1C]

Compound 2551 was prepared on a 50 μmol scale. The yield of the product was 21.3 mg, and its estimated purity by LCMS analysis was 95%. Analysis condition B: Retention time=1.68 min; ESI-MS(+) m/z [M+H]⁺: 1996.4.

### Preparation of Compound 2552

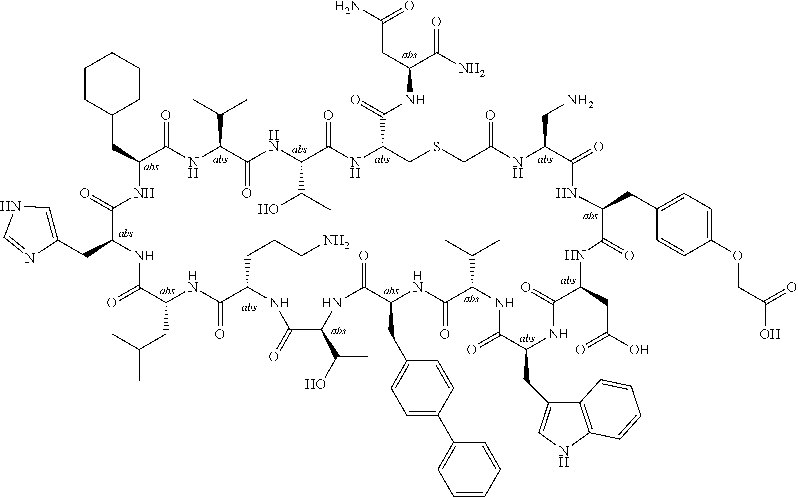 [SMILES: CC(C)C[C@H]1NC(=O)[C@H](CCCN)NC(=O)[C@H](C(C)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CN)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](Cc2c[nH]cn2)NC1=O]

Compound 2552 was prepared on a 50 μmol scale. The yield of the product was 49.8 mg, and its estimated purity by LCMS analysis was 100%. Analysis condition A: Retention time=1.89 min; ESI-MS(+) m/z [M+2H]²⁺: 1013.1.

### Preparation of Compound 2553

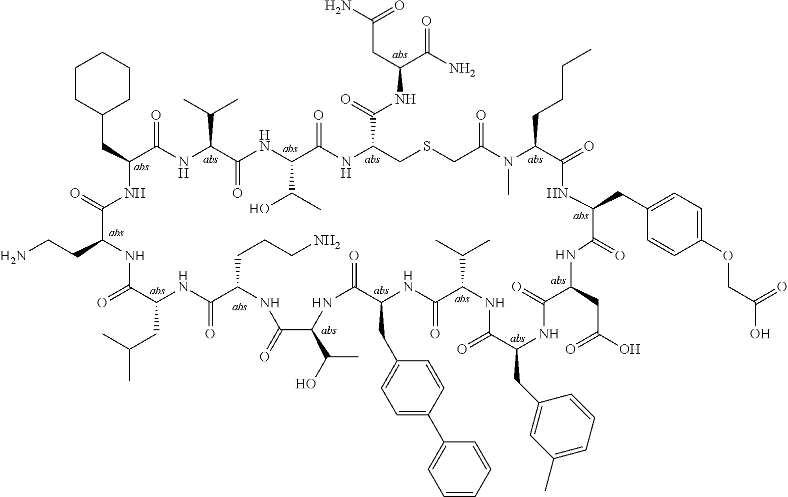 [SMILES: CCCC[C@H]1C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](C(C)O)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](C(C)O)C(=O)N[C@H](C(=O)N[C@@H](CC(N)=O)C(N)=O)CSCC(=O)N1C]

Compound 2553 was prepared on a 50 μmol scale. The yield of the product was 21.2 mg, and its estimated purity by LCMS analysis was 100%. Analysis condition B: Retention time=1.77 min; ESI-MS(+) m/z [M+2H]²⁺: 1002.2.

### Preparation of Compound 2554

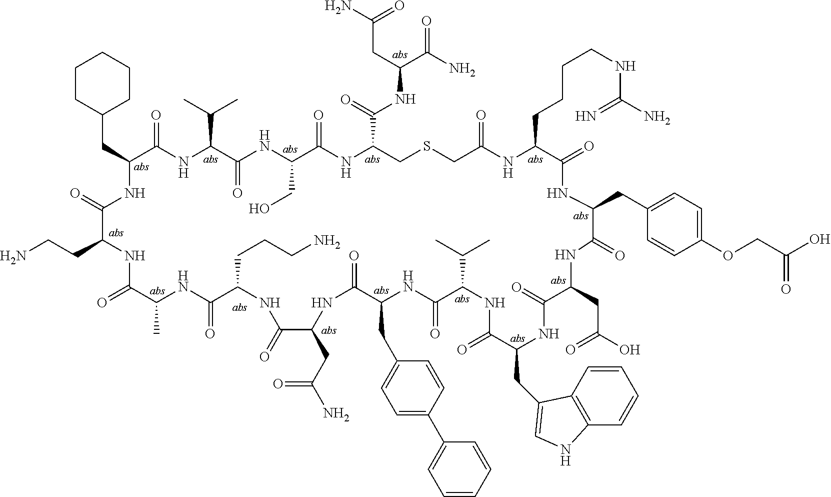 [SMILES: CC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](C)NC(=O)[C@H](CCCN)NC(=O)[C@H](CC(N)=O)NC(=O)[C@H](Cc2ccc(-c3ccccc3)cc2)NC1=O]

Compound 2554 was prepared on a 25 μmol scale. The yield of the product was 6.3 mg, and its estimated purity by LCMS analysis was 98.2%. Analysis condition B: Retention time=1.48 min; ESI-MS(+) m/z [M+2H]²⁺: 1014.4.

### Preparation of Compound 2555

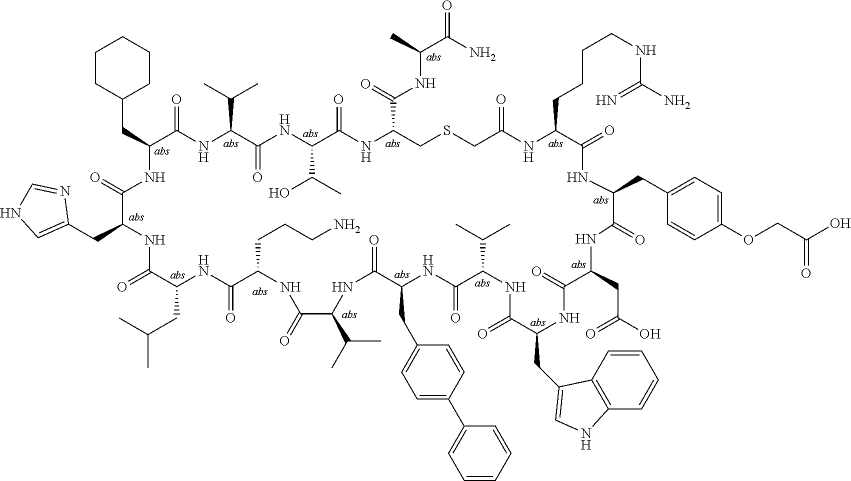 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N[C@@H](C)C(N)=O)NC(=O)[C@H](C(C)O)NC(=O)[C@H](C(C)C)NC(=O)[C@H](CC2CCCCC2)NC(=O)[C@H](Cc2c[nH]cn2)NC1=O]

Compound 2555 was prepared on a 25 μmol scale. The yield of the product was 10.9 mg, and its estimated purity by LCMS analysis was 100%. Analysis condition A: Retention time=1.65 min; ESI-MS(+) m/z [M+2H]²⁺: 1033.

### Preparation of Compound 2556

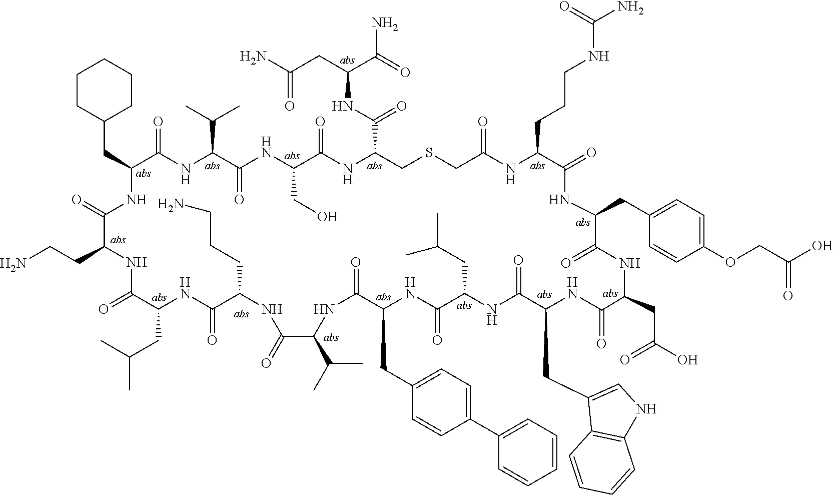 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCNC(N)=O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)C)NC(=O)[C@H](Cc2ccc(-c3ccccc3)cc2)NC1=O]

Compound 2556 was prepared on a 25 μmol scale. The yield of the product was 8 mg, and its estimated purity by LCMS analysis was 95%. Analysis condition B: Retention time=1.5 min; ESI-MS(+) m/z [M+2H]²⁺: 1035.9.

### Preparation of Compound 2557

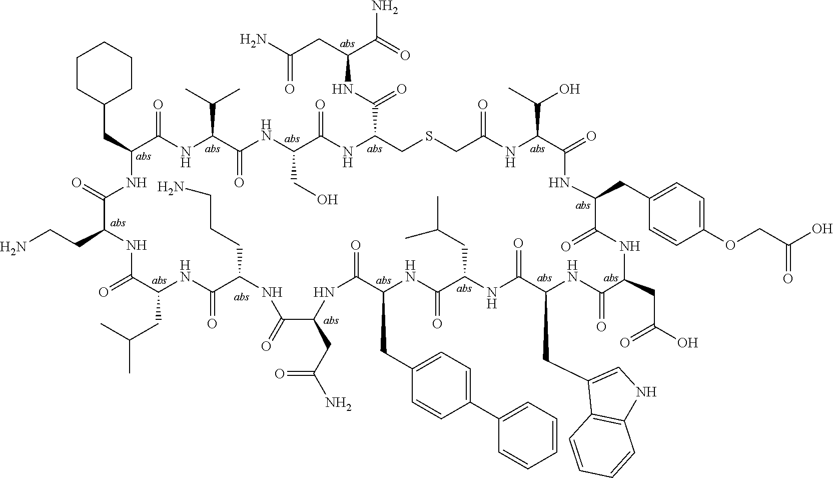 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CC(N)=O)NC(=O)[C@H](Cc2ccc(-c3ccccc3)cc2)NC1=O]

Compound 2557 was prepared on a 25 μmol scale. The yield of the product was 36 mg, and its estimated purity by LCMS analysis was 99%. Analysis condition B: Retention time=1.49 min; ESI-MS(+) m/z [M+3H]³⁺: 673.

### Preparation of Compound 2558

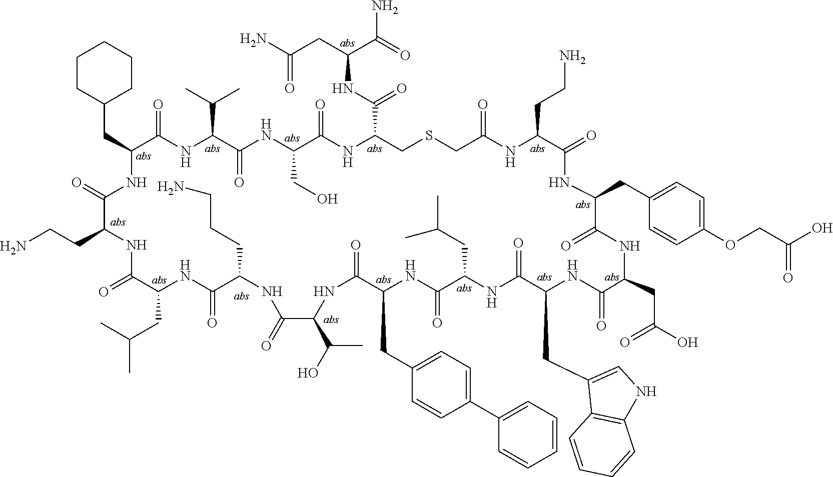 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)O)NC(=O)[C@H](Cc2ccc(-c3ccccc3)cc2)NC1=O]

Compound 2558 was prepared on a 25 μmol scale. The yield of the product was 10.4 mg, and its estimated purity by LCMS analysis was 97.9%. Analysis condition A: Retention time=1.55 min; ESI-MS(+) m/z [M+2H]²⁺: 1001.1.

### Preparation of Compound 2559

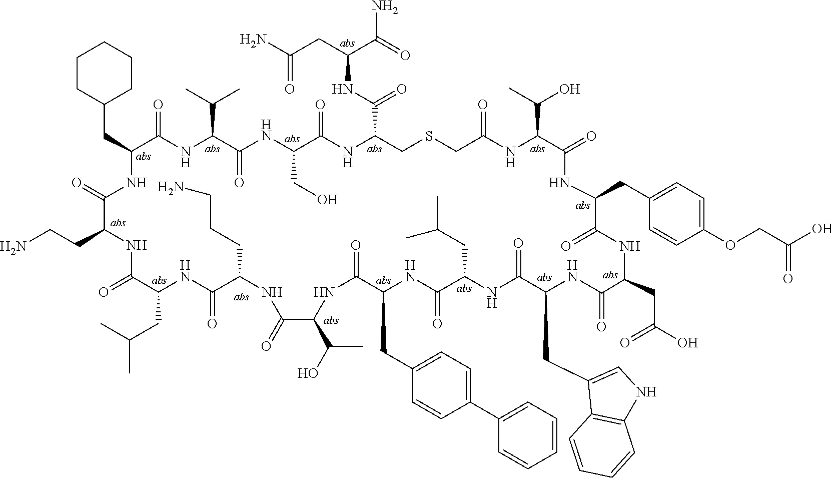 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](C(C)O)NC(=O)[C@H](Cc2ccc(-c3ccccc3)cc2)NC1=O]

Compound 2559 was prepared on a 25 μmol scale. The yield of the product was 8.3 mg, and its estimated purity by LCMS analysis was 95.1%. Analysis condition B: Retention time=1.41 min; ESI-MS(+) m/z [M+2H]²⁺: 1002.1.

### Preparation of Compound 2560

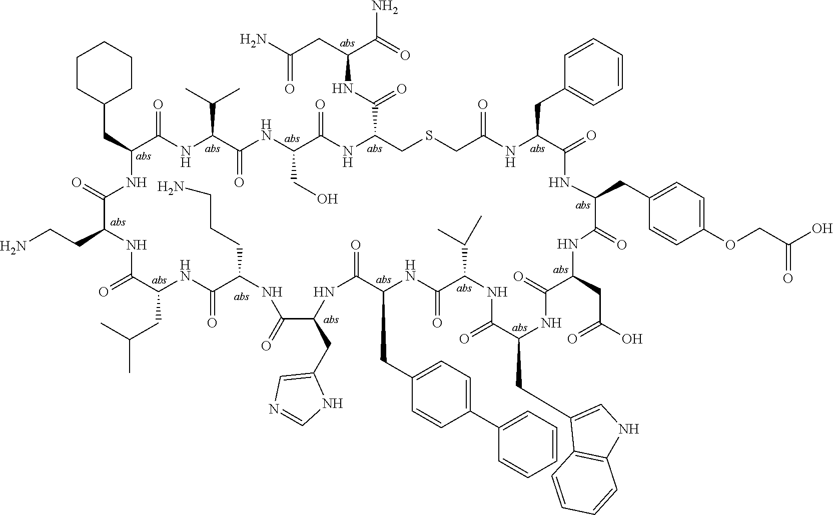 [SMILES: CC(C)C[C@H]1NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccccc2)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2560 was prepared on a 25 μmol scale. The yield of the product was 15.1 mg, and its estimated purity by LCMS analysis was 100%. Analysis condition A: Retention time=1.35 min; ESI-MS(+) m/z [M+2H]²⁺: 1036.1.

### Preparation of Compound 2561

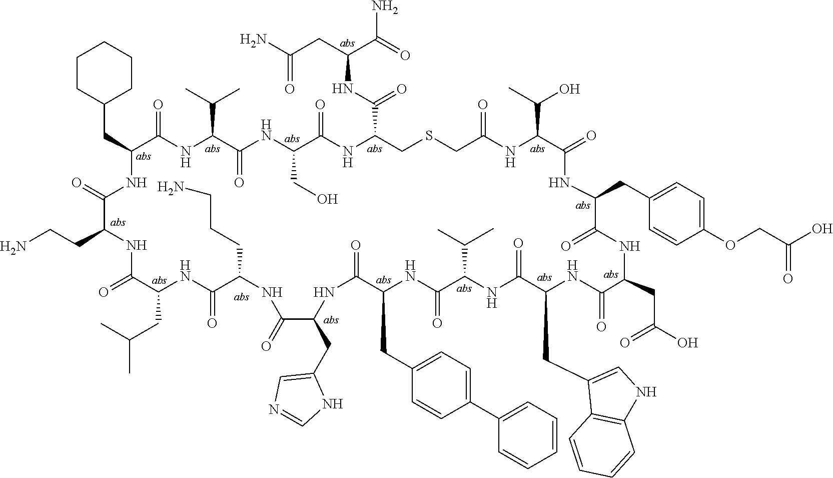 [SMILES: CC(C)C[C@H]1NC(=O)[C@H](CCCN)NC(=O)[C@H](Cc2cnc[nH]2)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2561 was prepared on a 25 μmol scale. The yield of the product was 8.6 mg, and its estimated purity by LCMS analysis was 95.6%. Analysis condition B: Retention time=1.29 min; ESI-MS(+) m/z [M+2H]²⁺: 1013.

### Preparation of Compound 2562

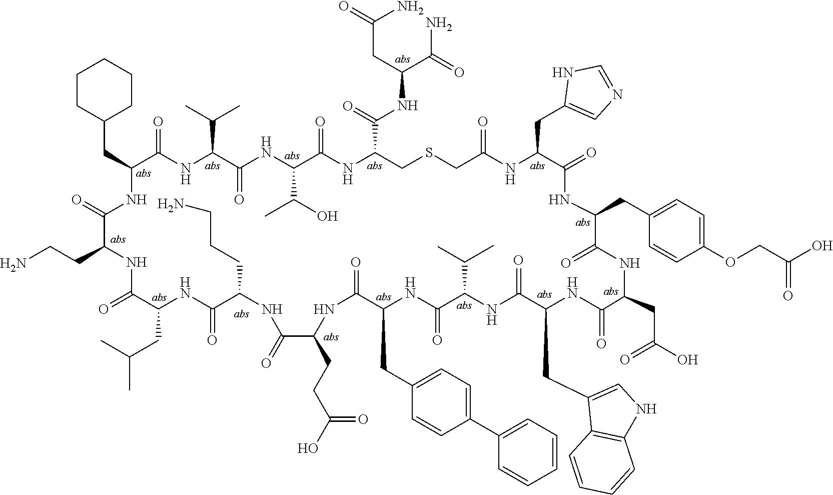 [SMILES: CC(C)C[C@H]1NC(=O)[C@H](CCCN)NC(=O)[C@H](CCC(=O)O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@@H](CC(N)=O)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2562 was prepared on a 50 μmol scale. The yield of the product was 13.7 mg, and its estimated purity by LCMS analysis was 98.5%. Analysis condition B: Retention time=1.48 min; ESI-MS(+) m/z [M+2H]²⁺: 1034.3.

### Preparation of Compound 2563

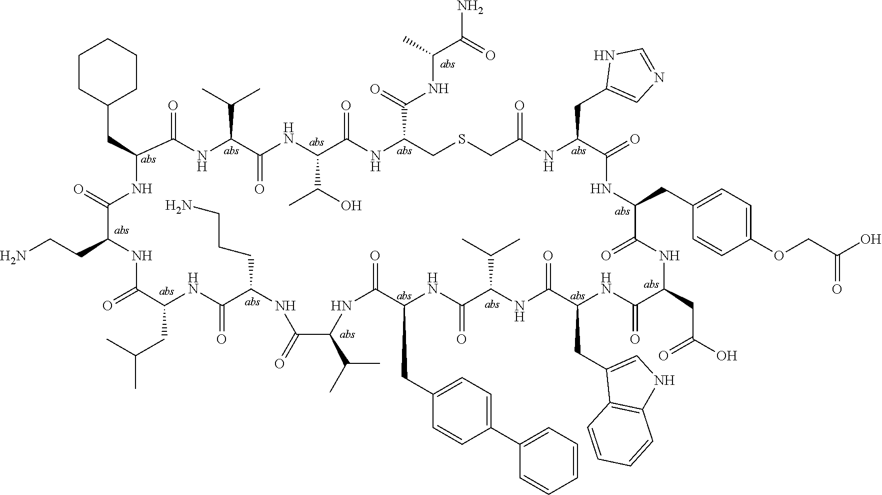 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@H](C)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2563 was prepared on a 50 μmol scale. The yield of the product was 12.1 mg, and its estimated purity by LCMS analysis was 99.2%. Analysis condition A: Retention time=1.53 min; ESI-MS(+) m/z [M+2H]²⁺: 997.2.

### Preparation of Compound 2564

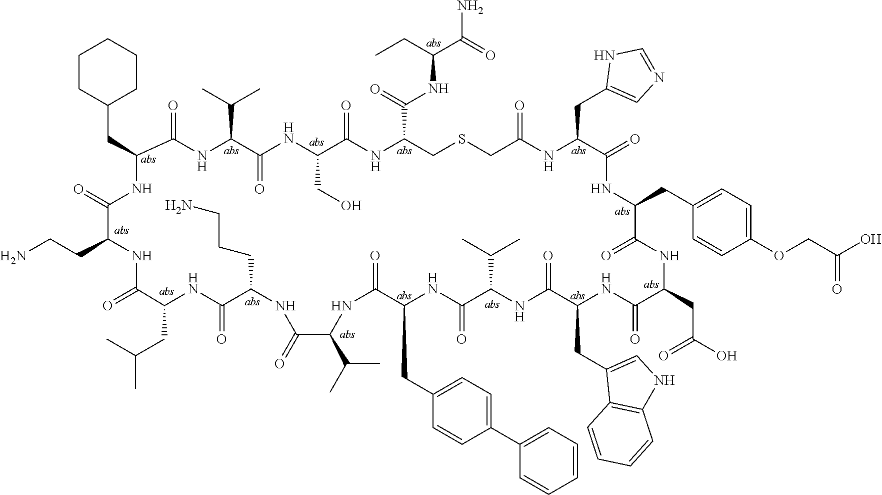 [SMILES: CC[C@H](NC(=O)[C@@H]1CSCC(=O)N[C@@H](Cc2cnc[nH]2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)C)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CO)C(=O)N1)C(N)=O]

Compound 2564 was prepared on a 50 μmol scale. The yield of the product was 7.5 mg, and its estimated purity by LCMS analysis was 98.6%. Analysis condition A: Retention time=1.72 min; ESI-MS(+) m/z [M+H]⁺: 1992.9.

### Preparation of Compound 2565

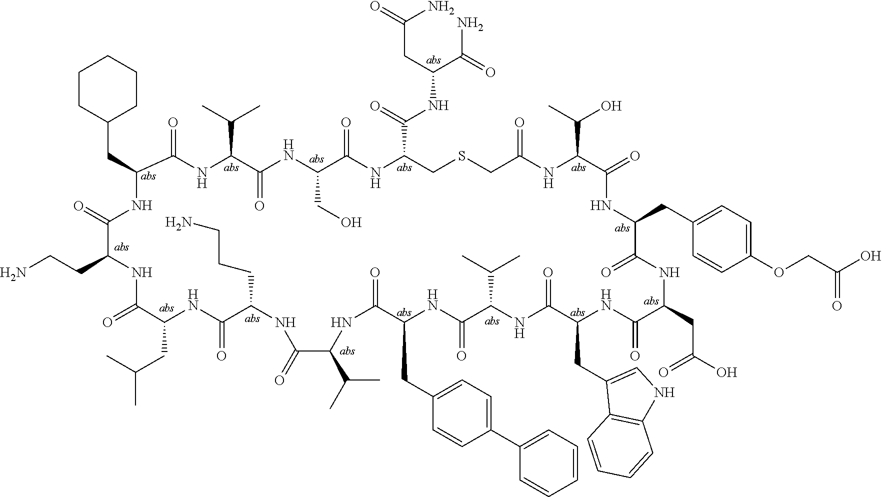 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2565 was prepared on a 50 μmol scale. The yield of the product was 21.4 mg, and its estimated purity by LCMS analysis was 97.3%. Analysis condition B: Retention time=1.62 min; ESI-MS(+) m/z [M+H]⁺: 1986.

### Preparation of Compound 2566

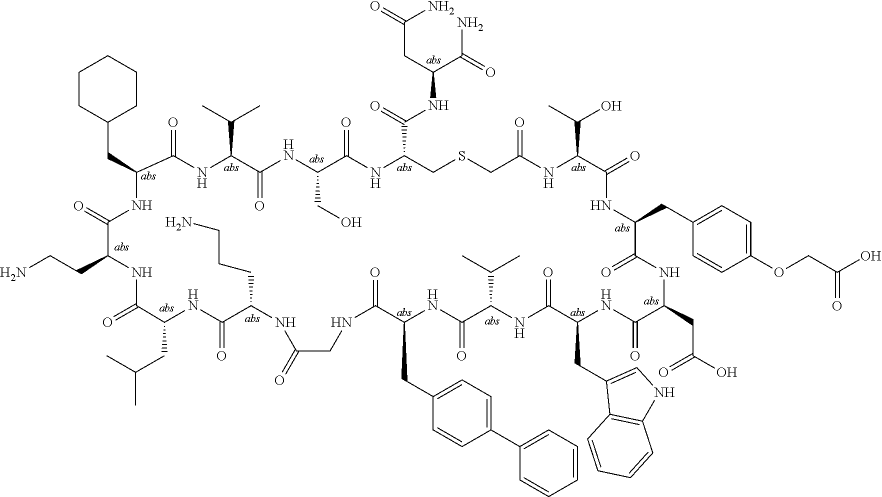 [SMILES: CC(C)C[C@H]1NC(=O)[C@H](CCCN)NC(=O)CNC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2566 was prepared on a 50 μmol scale. The yield of the product was 11.5 mg, and its estimated purity by LCMS analysis was 100%. Analysis condition A: Retention time=1.44 min; ESI-MS(+) m/z [M+2H]²⁺: 973.2.

### Preparation of Compound 2567

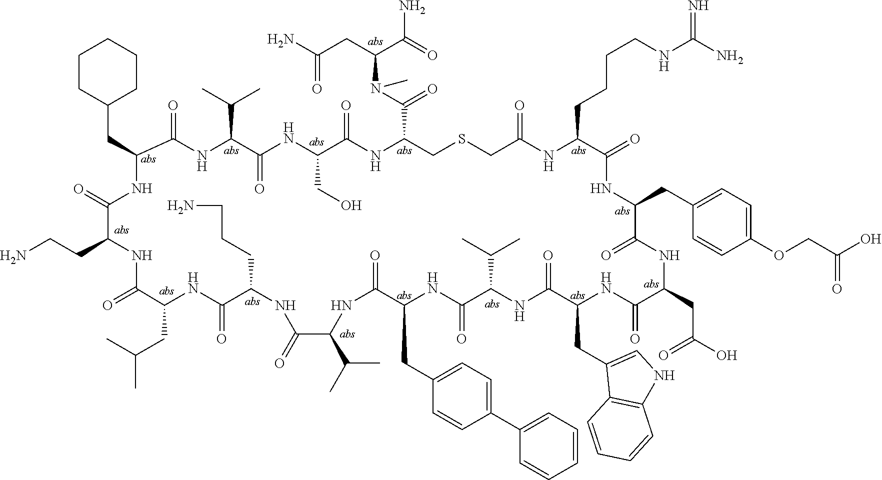 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N(C)[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2567 was prepared on a 50 μmol scale. The yield of the product was 4.1 mg, and its estimated purity by LCMS analysis was 95.9%. Analysis condition B: Retention time=1.55 min; ESI-MS(+) m/z [M+2H]²⁺: 1035.

### Preparation of Compound 2568

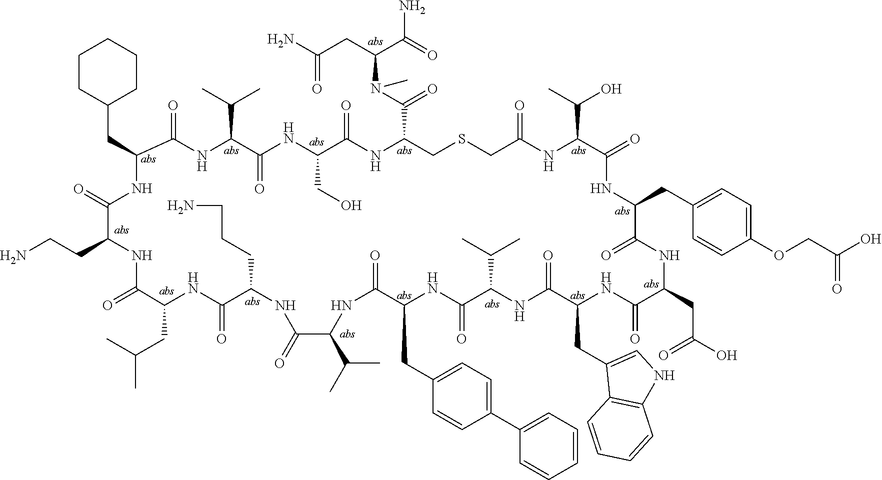 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)[C@@H](CC(N)=O)C(N)=O)NC(=O)[C@H](CO)NC(=O)[C@H](C(C)C)NC(=O)[C@H](CC2CCCCC2)NC(=O)[C@H](CCN)NC1=O]

Compound 2568 was prepared on a 50 μmol scale. The yield of the product was 6.7 mg, and its estimated purity by LCMS analysis was 98%. Analysis condition B: Retention time=1.66 min; ESI-MS(+) m/z [M+2H]²⁺: 1001.

### Preparation of Compound 2569

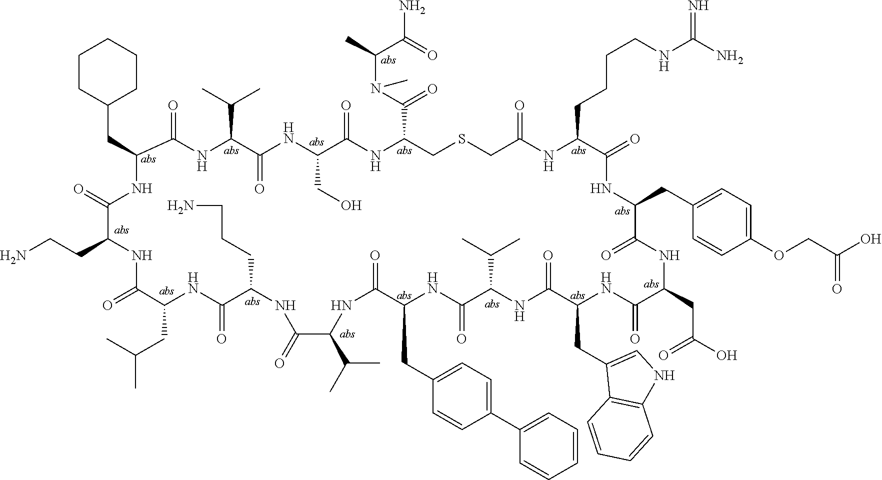 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(=O)N(C)[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2569 was prepared on a 50 μmol scale. The yield of the product was 5.3 mg, and its estimated purity by LCMS analysis was 100%. Analysis condition A: Retention time=1.61 min; ESI-MS(+) m/z [M+2H]²⁺: 1014.

### Preparation of Compound 2570

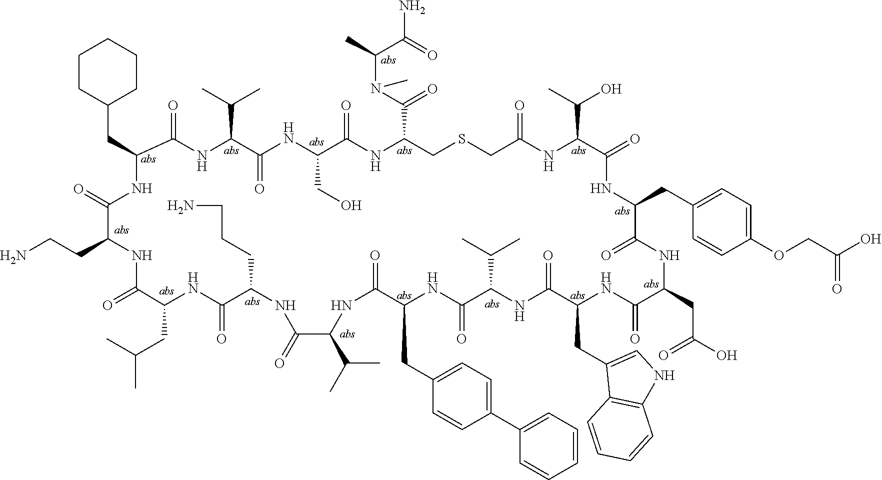 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)[C@@H](C)C(N)=O)NC(=O)[C@H](CO)NC(=O)[C@H](C(C)C)NC(=O)[C@H](CC2CCCCC2)NC(=O)[C@H](CCN)NC1=O]

Compound 2570 was prepared on a 50 μmol scale. The yield of the product was 8.8 mg, and its estimated purity by LCMS analysis was 96.6%. Analysis condition A: Retention time=1.71 min; ESI-MS(+) m/z [M+H]⁺: 1957.2.

### Preparation of Compound 2571

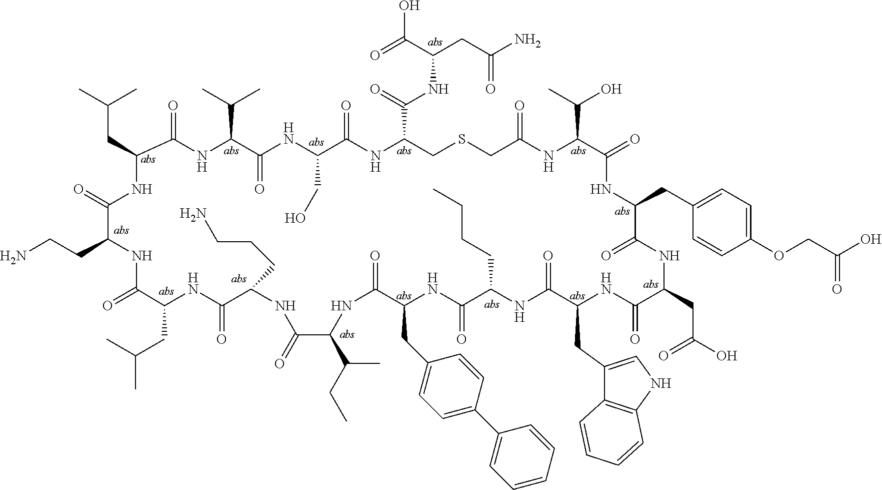 [SMILES: CCCC[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC1=O]

Compound 2571 was prepared on a 50 μmol scale. The yield of the product was 8.1 mg, and its estimated purity by LCMS analysis was 96.8%. Analysis condition B: Retention time=1.56 min; ESI-MS(+) m/z [M+H]⁺: 1974.9.

### Preparation of Compound 2572

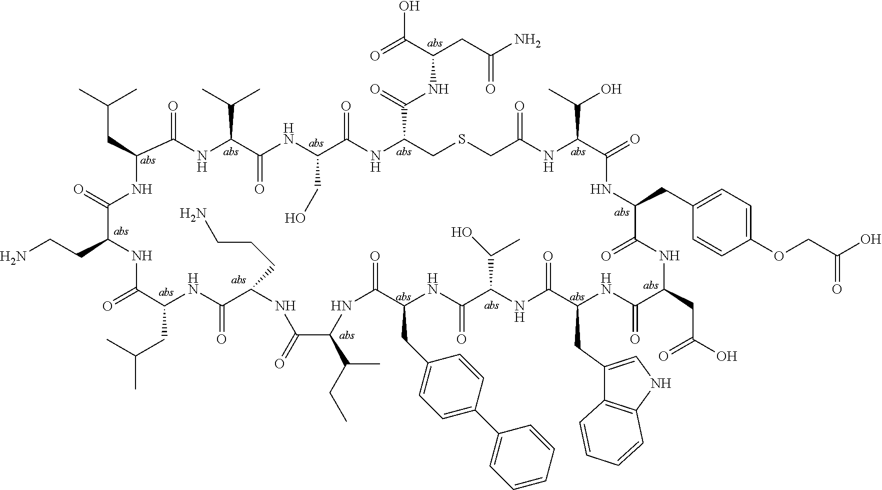 [SMILES: CCC(C)[C@@H]1NC(=O)[C@H](Cc2ccc(-c3ccccc3)cc2)NC(=O)[C@H](C(C)O)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2572 was prepared on a 50 μmol scale. The yield of the product was 16.2 mg, and its estimated purity by LCMS analysis was 93.3%. Analysis condition A: Retention time=1.58 min; ESI-MS(+) m/z [M+H]⁺: 1964.1.

### Preparation of Compound 2573

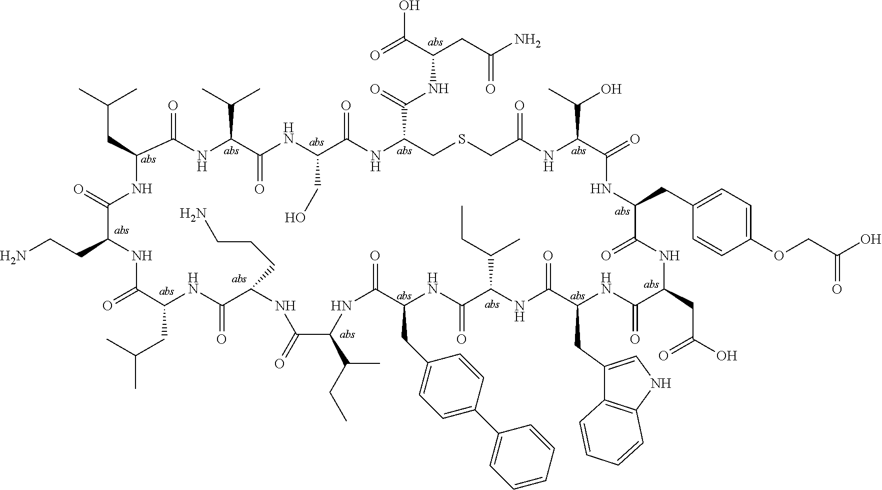 [SMILES: CCC(C)[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC1=O]

Compound 2573 was prepared on a 50 μmol scale. The yield of the product was 18.1 mg, and its estimated purity by LCMS analysis was 90.7%. Analysis condition A: Retention time=1.45 min; ESI-MS(+) m/z [M+2H]²⁺: 988.1.

### Preparation of Compound 2574

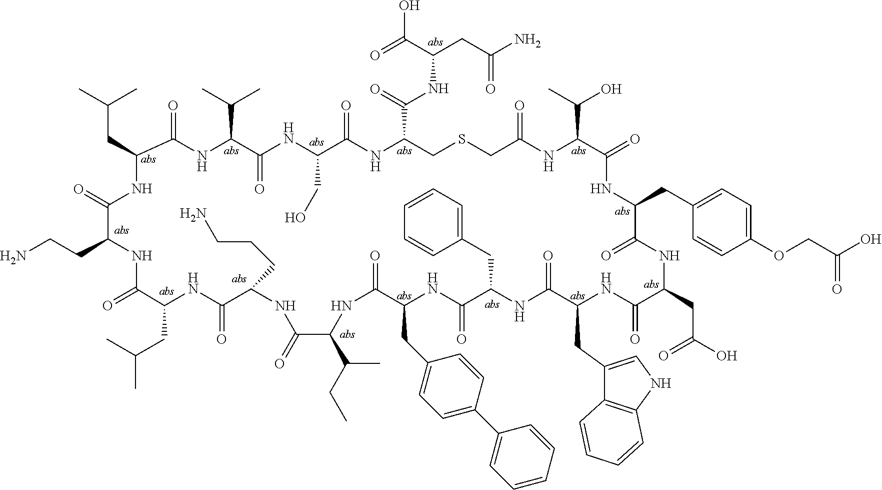 [SMILES: CCC(C)[C@@H]1NC(=O)[C@H](Cc2ccc(-c3ccccc3)cc2)NC(=O)[C@H](Cc2ccccc2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2574 was prepared on a 50 μmol scale. The yield of the product was 17.1 mg, and its estimated purity by LCMS analysis was 100%. Analysis condition A: Retention time=1.66, 1.72 min; ESI-MS(+) m/z [M+2H]²⁺: 1006.

### Preparation of Compound 2575

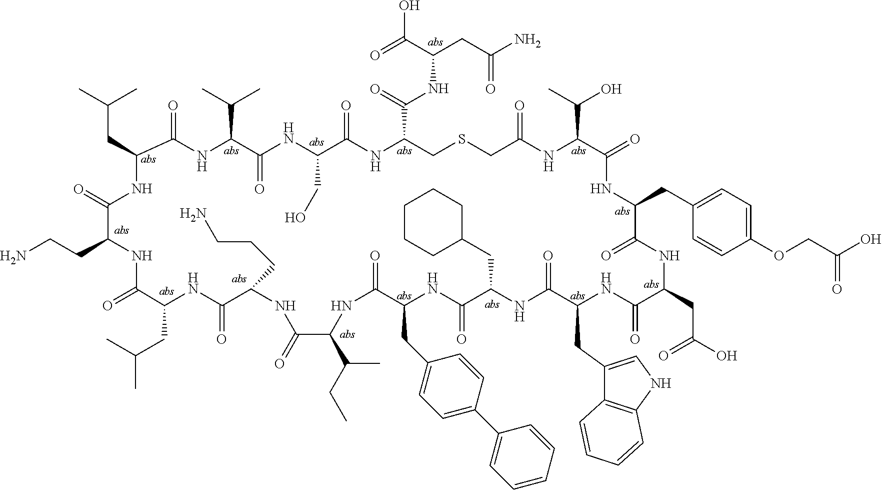 [SMILES: CCC(C)[C@@H]1NC(=O)[C@H](Cc2ccc(-c3ccccc3)cc2)NC(=O)[C@H](CC2CCCCC2)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2575 was prepared on a 50 μmol scale. The yield of the product was 4.5 mg, and its estimated purity by LCMS analysis was 96.7%. Analysis condition A: Retention time=1.76 min; ESI-MS(+) m/z [M+2H]²⁺: 1008.9.

### Preparation of Compound 2576

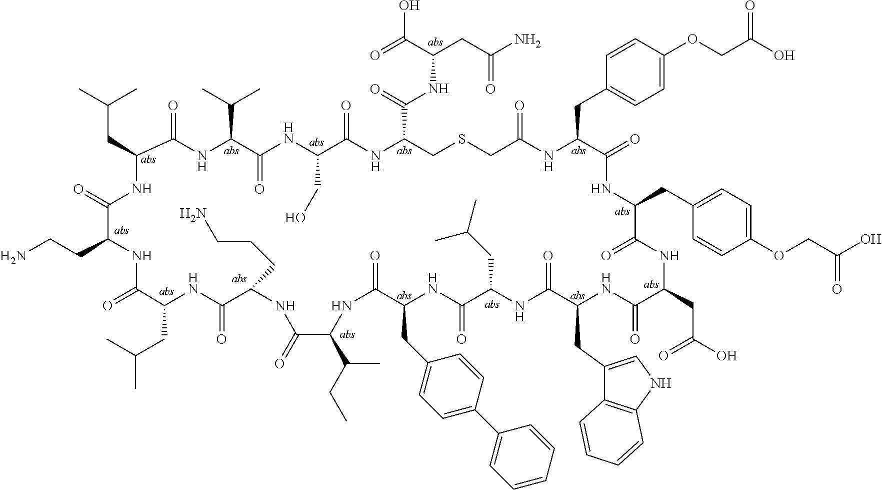 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(OCC(=O)O)cc2)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2576 was prepared on a 50 μmol scale. The yield of the product was 16.3 mg, and its estimated purity by LCMS analysis was 86.3%. Analysis condition A: Retention time=1.64 min; ESI-MS(+) m/z [M+2H]²⁺: 1048.

### Preparation of Compound 2577

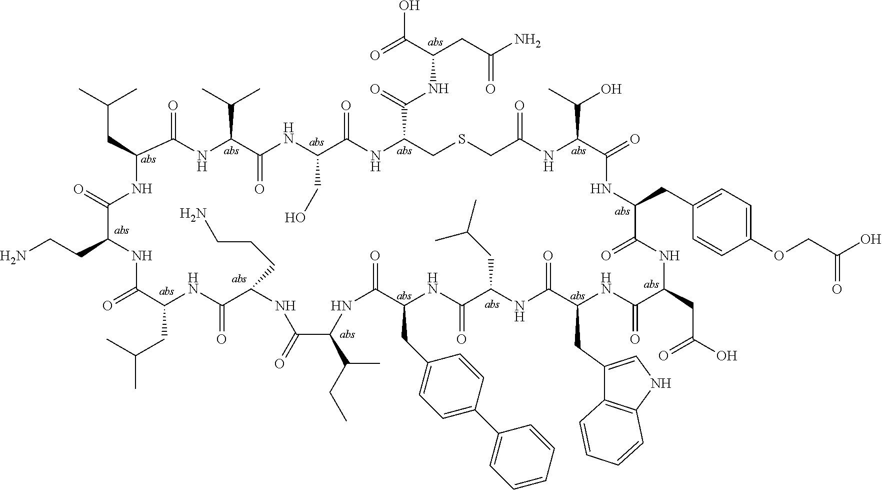 [SMILES: CCC(C)[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2577 was prepared on a 50 μmol scale. The yield of the product was 5.6 mg, and its estimated purity by LCMS analysis was 96.6%. Analysis condition A: Retention time=1.64 min; ESI-MS(+) m/z [M+H]⁺: 1975.6.

### Preparation of Compound 2578

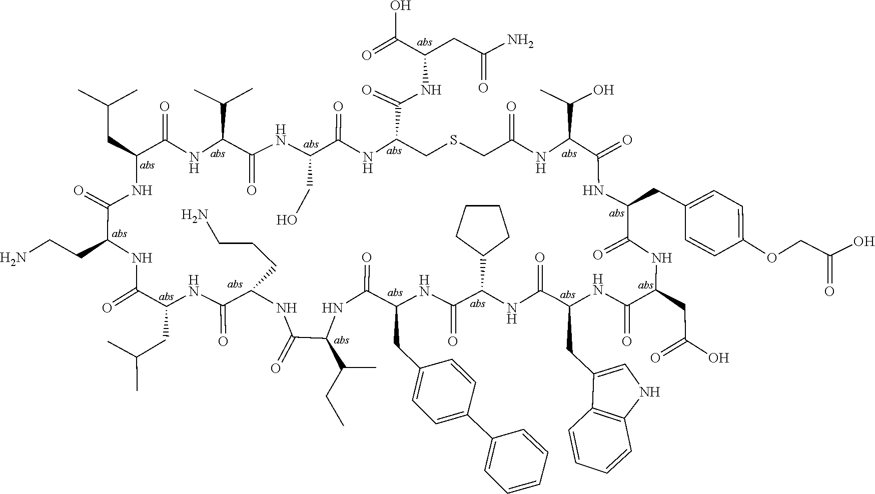 [SMILES: CCC(C)[C@@H]1NC(=O)[C@H](Cc2ccc(-c3ccccc3)cc2)NC(=O)[C@H](C2CCCC2)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2578 was prepared on a 50 μmol scale. The yield of the product was 9.4 mg, and its estimated purity by LCMS analysis was 88.2%. Analysis condition A: Retention time=1.5 min; ESI-MS(+) m/z [M+2H]²⁺: 994.2.

### Preparation of Compound 2579

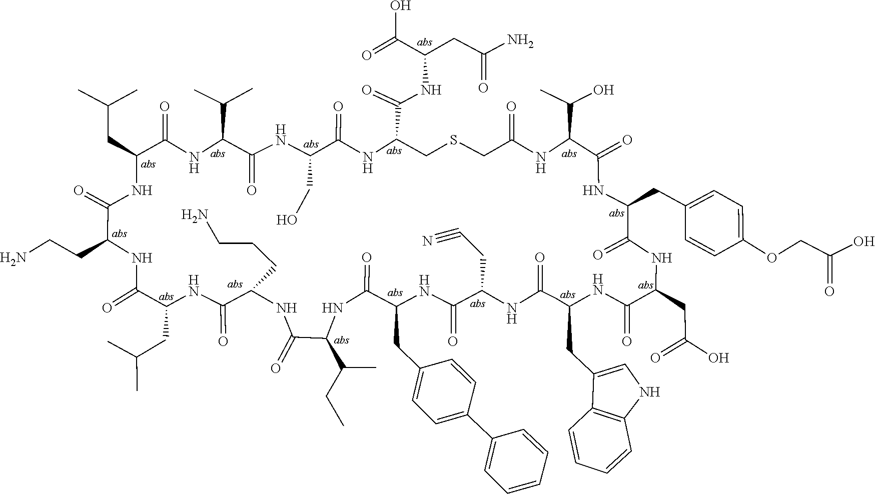 [SMILES: CCC(C)[C@@H]1NC(=O)[C@H](Cc2ccc(-c3ccccc3)cc2)NC(=O)[C@H](CC#N)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2579 was prepared on a 50 μmol scale. The yield of the product was 12.8 mg, and its estimated purity by LCMS analysis was 93.2%. Analysis condition A: Retention time=1.58 min; ESI-MS(+) m/z [M+H]⁺: 1959.1.

### Preparation of Compound 2580

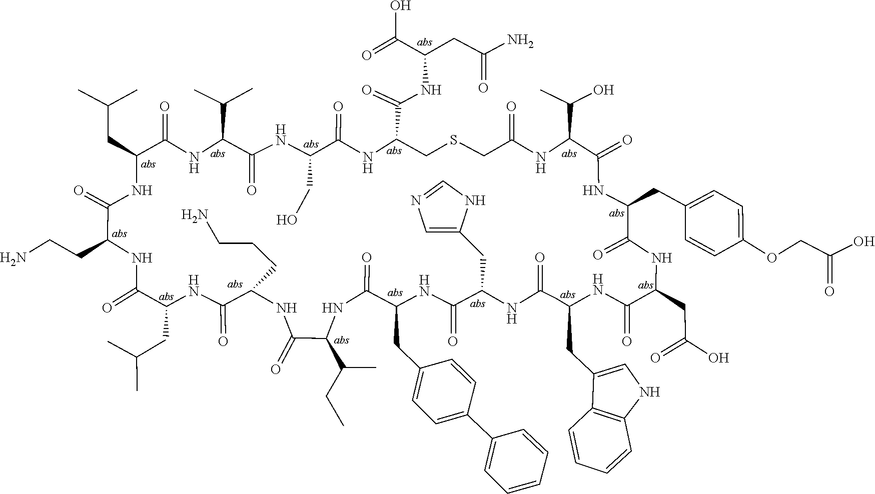 [SMILES: CCC(C)[C@@H]1NC(=O)[C@H](Cc2ccc(-c3ccccc3)cc2)NC(=O)[C@H](Cc2cnc[nH]2)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2580 was prepared on a 50 μmol scale. The yield of the product was 8.4 mg, and its estimated purity by LCMS analysis was 99%. Analysis condition B: Retention time=1.31 min; ESI-MS(+) m/z [M+3H]³⁺: 667.1.

### Preparation of Compound 2581

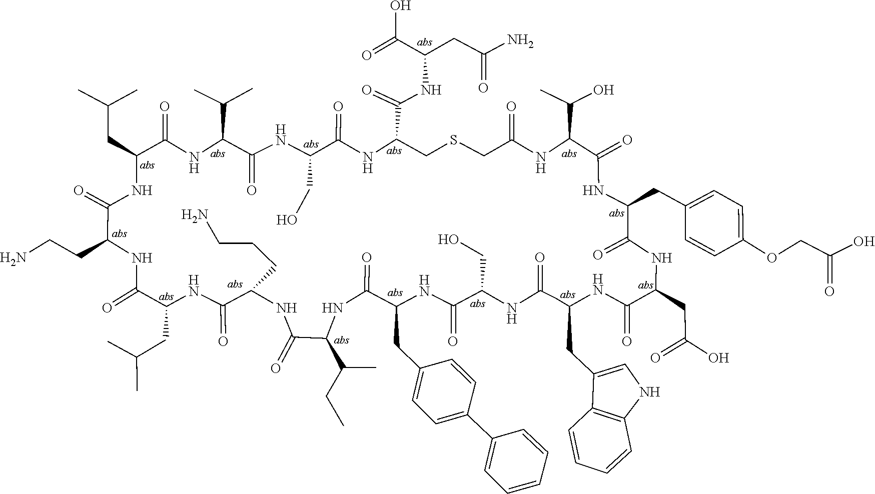 [SMILES: CCC(C)[C@@H]1NC(=O)[C@H](Cc2ccc(-c3ccccc3)cc2)NC(=O)[C@H](CO)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2581 was prepared on a 50 μmol scale. The yield of the product was 5 mg, and its estimated purity by LCMS analysis was 98.6%. Analysis condition A: Retention time=1.49 min; ESI-MS(+) m/z [M+2H]²⁺: 975.2.

### Preparation of Compound 2582

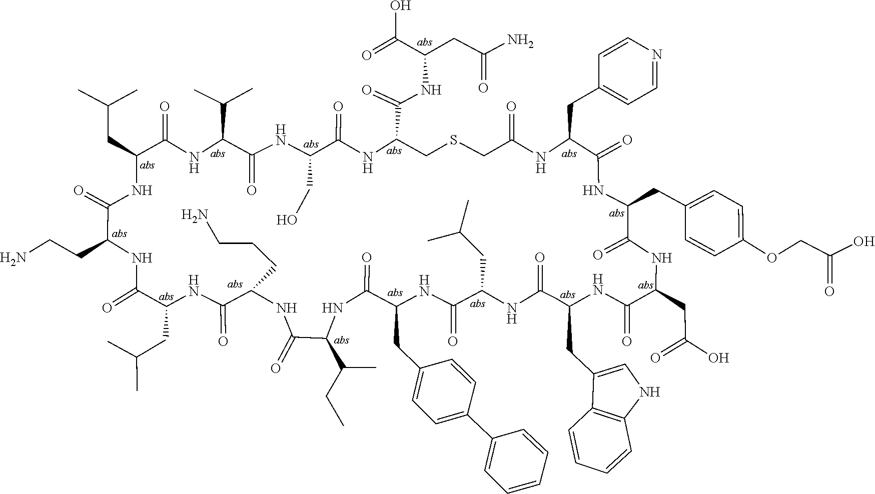 [SMILES: CCC(C)[C@@H]1NC(=O)[C@H](Cc2ccc(-c3ccccc3)cc2)NC(=O)[C@H](CC(C)C)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CC(N)=O)C(=O)O)NC(=O)[C@H](CO)NC(=O)[C@H](C(C)C)NC(=O)[C@H](CC(C)C)NC(=O)[C@H](CCN)NC(=O)[C@@H](CC(C)C)NC(=O)[C@H](CCCN)NC1=O]

Compound 2582 was prepared on a 50 μmol scale. The yield of the product was 7.6 mg, and its estimated purity by LCMS analysis was 96.3%. Analysis condition A: Retention time=1.62 min; ESI-MS(+) m/z [M+2H]²⁺: 1012.1.

### Preparation of Compound 2583

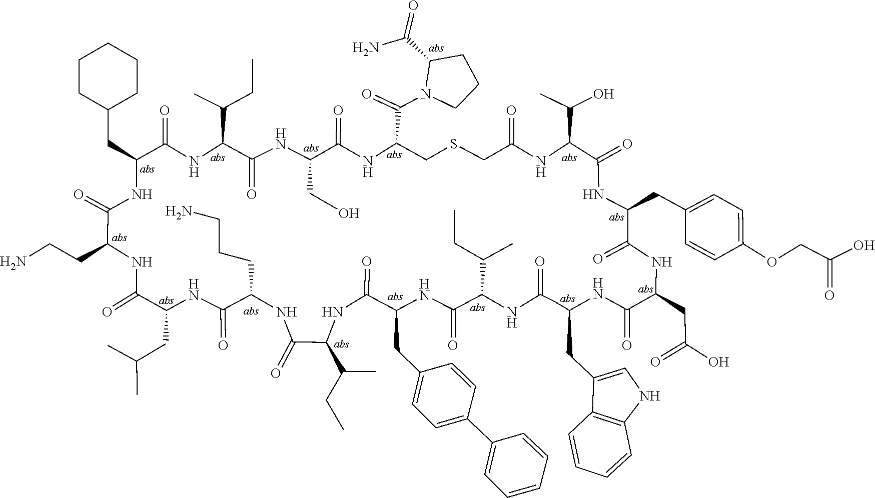 [SMILES: CCC(C)[C@@H]1NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N2CCC[C@H]2C(N)=O)NC(=O)[C@H](CO)NC(=O)[C@H](C(C)CC)NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC1=O]

Compound 2583 was prepared on a 50 μmol scale. The yield of the product was 7.2 mg, and its estimated purity by LCMS analysis was 95.3%. Analysis condition B: Retention time=1.73 min; ESI-MS(+) m/z [M+2H]²⁺: 1006.2.

### Preparation of Compound 2584

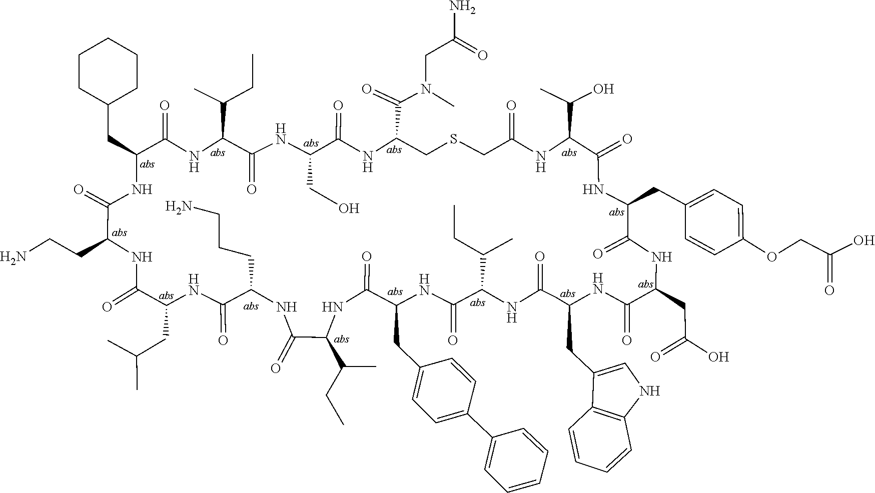 [SMILES: CCC(C)[C@@H]1NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N(C)CC(N)=O)NC(=O)[C@H](CO)NC(=O)[C@H](C(C)CC)NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC1=O]

Compound 2584 was prepared on a 50 μmol scale. The yield of the product was 11.9 mg, and its estimated purity by LCMS analysis was 100%. Analysis condition A: Retention time=1.78 min; ESI-MS(+) m/z [M+H]⁺: 1985.1.

### Preparation of Compound 2585

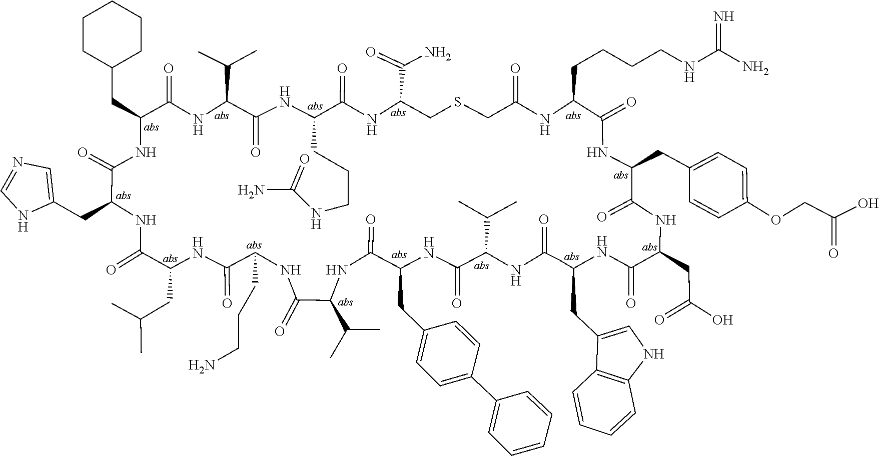 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)C[C@H](Cc2c[nH]c3ccccc23)NC(=O)[C@H](CC(=O)O)NC(=O)[C@H](Cc2ccc(OCC(=O)O)cc2)NC(=O)[C@H](CCCCNC(=N)N)NC(=O)CSC[C@@H](C(N)=O)NC(=O)[C@H](CCCNC(N)=O)NC(=O)[C@H](C(C)C)NC(=O)[C@H](CC2CCCCC2)NC(=O)[C@H](Cc2cnc[nH]2)NC1=O]

Compound 2585 was prepared on a 50 μmol scale. The yield of the product was 23.5 mg, and its estimated purity by LCMS analysis was 96.7%. Analysis condition A: Retention time=1.6, 1.66 min; ESI-MS(+) m/z [M+2H]²⁺: 1025.08, 1025.08.

### Preparation of Compound 2586

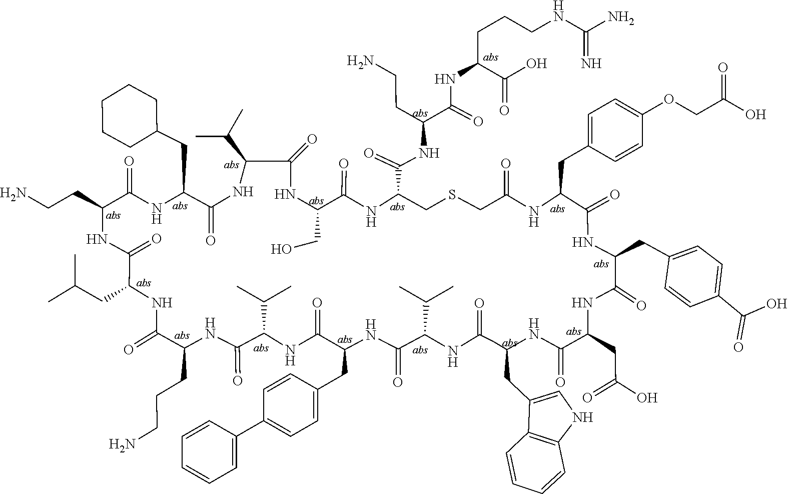 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)C[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)N[C@@H](CCCNC(=N)N)C(=O)O)NC(=O)[C@H](CO)NC(=O)[C@H](C(C)C)NC(=O)[C@H](CC2CCCCC2)NC(=O)[C@H](CCN)NC1=O]

Compound 2586 was prepared on a 50 μmol scale. The yield of the product was 6.2 mg, and its estimated purity by LCMS analysis was 95.7%. Analysis condition B: Retention time=1.46 min; ESI-MS(+) m/z [M+3H]³⁺: 740.8.

### Preparation of Compound 2587

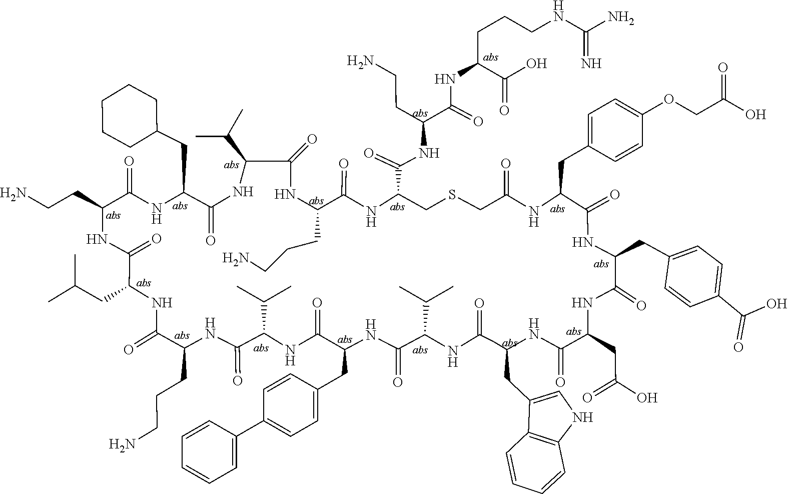 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)C[C@H](Cc2c[nH]c3ccccc23)NC(=O)[C@H](CC(=O)O)NC(=O)[C@H](Cc2ccc(C(=O)O)cc2)NC(=O)[C@H](Cc2ccc(OCC(=O)O)cc2)NC(=O)CSC[C@@H](C(=O)N[C@@H](CCN)C(=O)N[C@@H](CCCNC(=N)N)C(=O)O)NC(=O)[C@H](CCCN)NC(=O)[C@H](C(C)C)NC(=O)[C@H](CC2CCCCC2)NC(=O)[C@H](CCN)NC1=O]

Compound 2587 was prepared on a 50 μmol scale. The yield of the product was 17.9 mg, and its estimated purity by LCMS analysis was 100%. Analysis condition A: Retention time=1.51 min; ESI-MS(+) m/z [M+3H]³⁺: 749.4.

### Preparation of Compound 2588

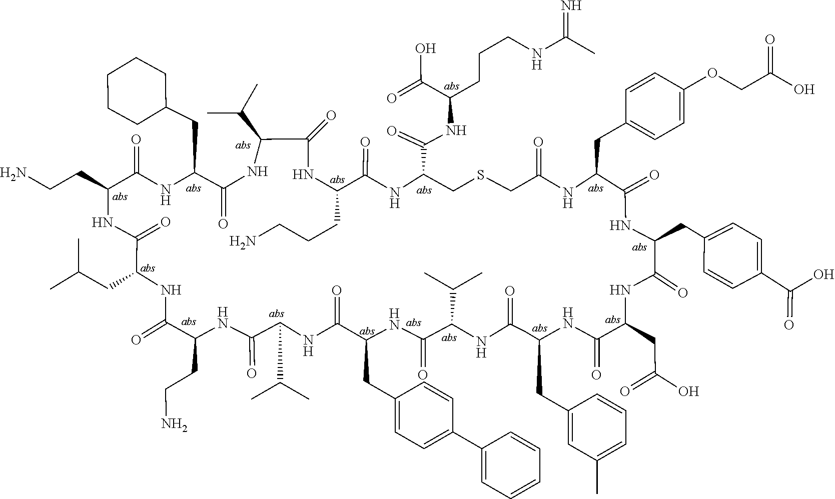

Compound 2588 was prepared on a 50 μmol scale. The yield of the product was 12.1 mg, and its estimated purity by LCMS analysis was 100%. Analysis condition A: Retention time=1.61 min; ESI-MS(+) m/z [M+3H]³⁺: 703.

### Preparation of Compound 2589

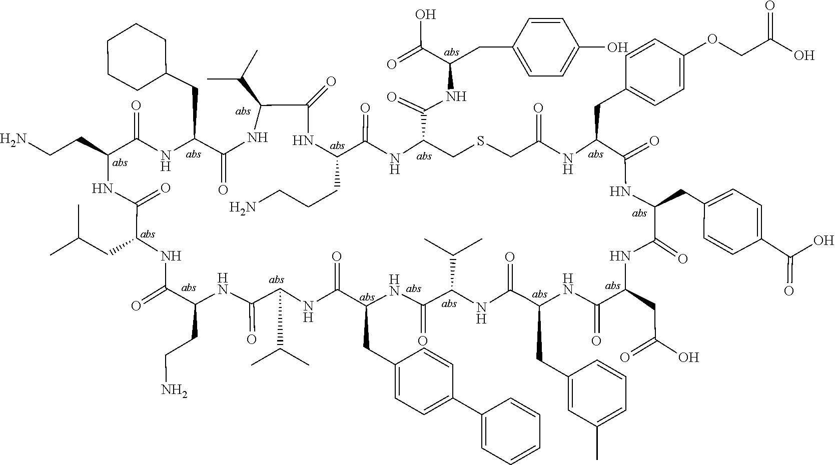

Compound 2589 was prepared on a 50 μmol scale. The yield of the product was 6.1 mg, and its estimated purity by LCMS analysis was 96.9%. Analysis condition B: Retention time=1.5 min; ESI-MS(+) m/z [M+2H]²⁺: 1058.1.

### Preparation of Compound 2590

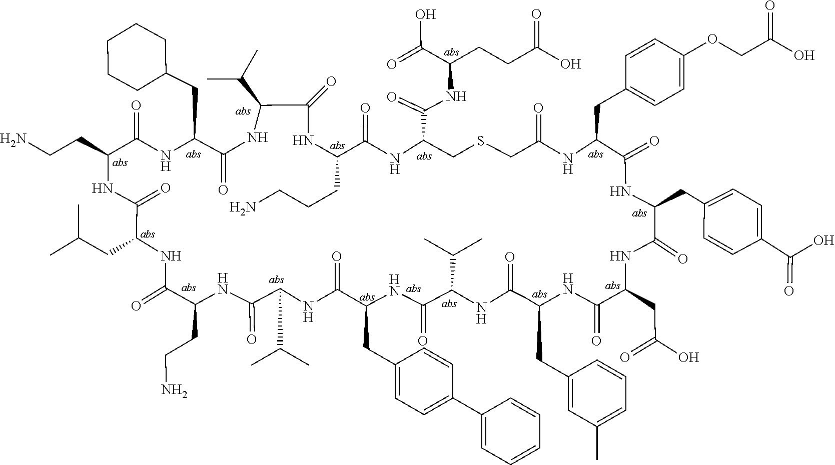

Compound 2590 was prepared on a 50 μmol scale. The yield of the product was 20.5 mg, and its estimated purity by LCMS analysis was 91.9%. Analysis condition B: Retention time=1.47 min; ESI-MS(+) m/z [M+2H]²⁺: 1041.1.

### Preparation of Compound 2591

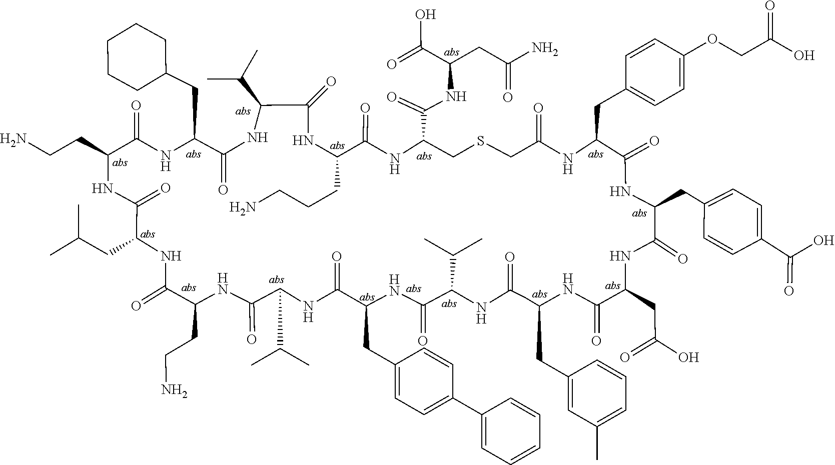

Compound 2591 was prepared on a 50 μmol scale. The yield of the product was 4.4 mg, and its estimated purity by LCMS analysis was 99%. Analysis condition B: Retention time=1.61 min; ESI-MS(+) m/z [M+2H]²⁺: 1031.8.

### Preparation of Compound 2592

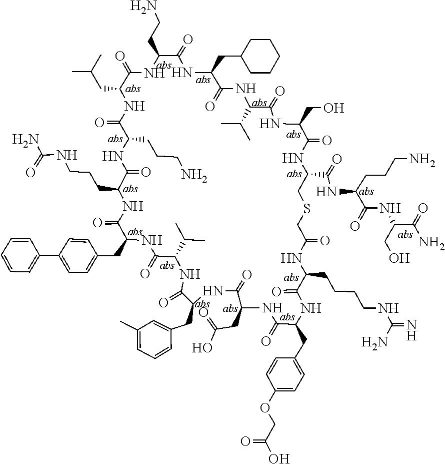

Compound 2592 was prepared on a 50 μmol scale. The yield of the product was 3.3 mg, and its estimated purity by LCMS analysis was 100%. Analysis condition A: Retention time=1.45 min; ESI-MS(+) m/z [M+3H]³⁺: 726.2.

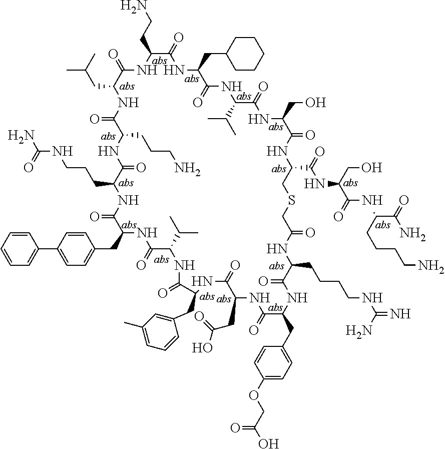 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CO)C(=O)N[C@@H](CCCCN)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2593 was prepared on a 50 μmol scale. The yield of the product was 23.3 mg, and its estimated purity by LCMS analysis was 95%. Analysis condition B: Retention time=1.32 min; ESI-MS(+) m/z [M+2H]²⁺: 1096.5.

### Preparation of Compound 2594

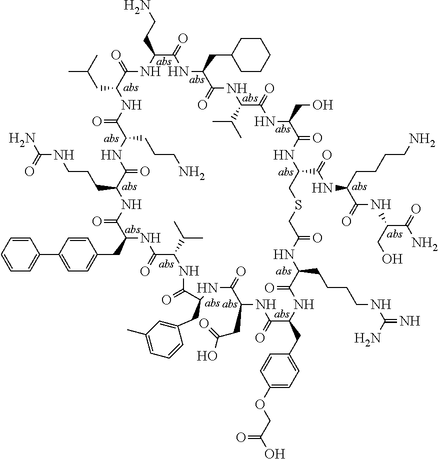 [SMILES: Cc1cccc(C[C@@H]2NC(=O)[C@H](CC(=O)O)NC(=O)[C@H](Cc3ccc(OCC(=O)O)cc3)NC(=O)[C@H](CCCCNC(=N)N)NC(=O)CSC[C@@H](C(=O)N[C@@H](CCCCN)C(=O)N[C@@H](CO)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2594 was prepared on a 50 μmol scale. The yield of the product was 4.1 mg, and its estimated purity by LCMS analysis was 98.3%. Analysis condition: Retention time=1.49 min; ESI-MS(+) m/z [M+2H]²⁺: 1095.3.

### Preparation of Compound 2595

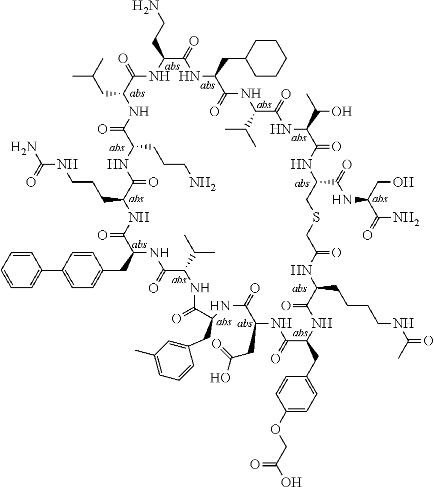 [SMILES: CC(=O)NCCCC[C@@H]1NC(=O)CSC[C@@H](C(=O)N[C@@H](CO)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2cccc(C)c2)NC(=O)[C@H](CC(=O)O)NC(=O)[C@H](Cc2ccc(OCC(=O)O)cc2)NC1=O]

Compound 2595 was prepared on a 50 μmol scale. The yield of the product was 30.1 mg, and its estimated purity by LCMS analysis was 95%. Analysis condition B: Retention time=1.5 min; ESI-MS(+) m/z [M+2H]²⁺: 1039.

### Preparation of Compound 2596

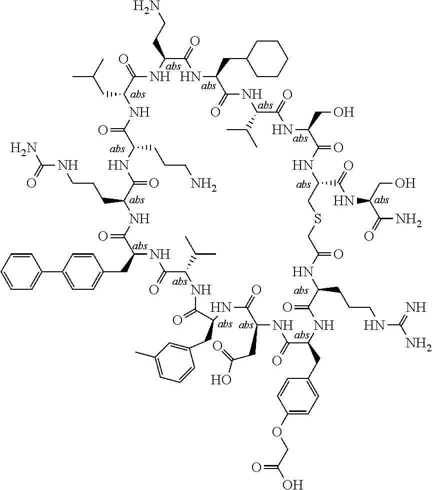 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CO)C(N)=O)NC(=O)[C@H](CO)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2596 was prepared on a 50 μmol scale. The yield of the product was 3.7 mg, and its estimated purity by LCMS analysis was 97.5%. Analysis condition: Retention time=1.55 min; ESI-MS(+) m/z [M+2H]²⁺: 1031.

### Preparation of Compound 2597

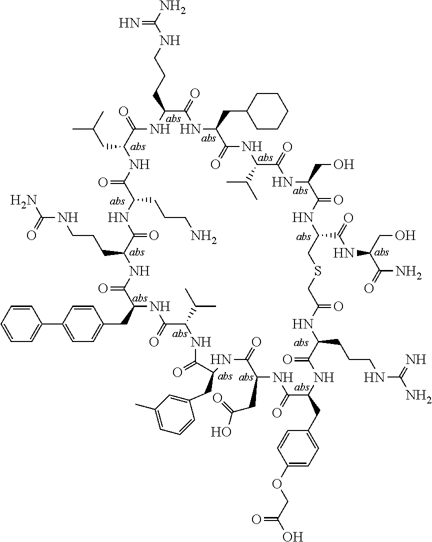 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CO)C(N)=O)NC(=O)[C@H](CO)NC(=O)[C@H](C(C)C)NC(=O)[C@H](CC3CCCCC3)NC(=O)[C@H](CCCNC(=N)N)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2597 was prepared on a 50 μmol scale. The yield of the product was 5.2 mg, and its estimated purity by LCMS analysis was 95.9%. Analysis condition: Retention time=1.54 min; ESI-MS(+) m/z [M+3H]³⁺: 702.1.

### Preparation of Compound 2598

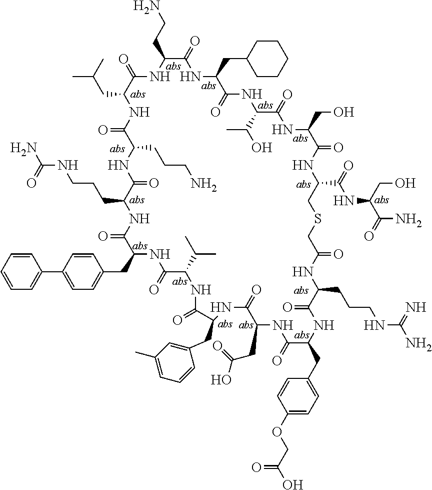 [SMILES: Cc1cccc(C[C@@H]2NC(=O)[C@H](CC(=O)O)NC(=O)[C@H](Cc3ccc(OCC(=O)O)cc3)NC(=O)[C@H](CCCNC(=N)N)NC(=O)CSC[C@@H](C(=O)N[C@@H](CO)C(N)=O)NC(=O)[C@H](CO)NC(=O)[C@H](C(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2598 was prepared on a 50 μmol scale. The yield of the product was 10.4 mg and its estimated purity by LCMS analysis was 97.7%. Analysis condition: Retention time=1.58 min; ESI-MS(+) m/z [M+3H]³⁺: 684.1.

### Preparation of Compound 2599

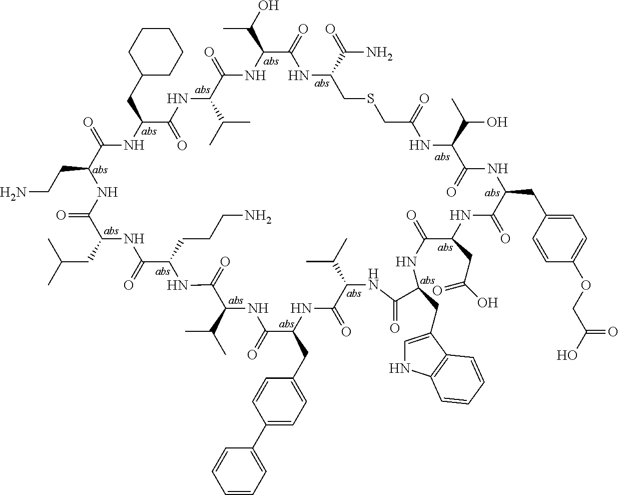 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](C(C)O)NC(=O)[C@H](C(C)C)NC(=O)[C@H](CC2CCCCC2)NC(=O)[C@H](CCN)NC1=O]

Compound 2599 was prepared on a 50 μmol scale. The yield of the product was 12.3 mg, and its estimated purity by LCMS analysis was 97.4%. Analysis condition: Retention time=1.74, 1.76 min; ESI-MS(+) m/z [M+2H]²⁺: 944.04, 944.04.

### Preparation of Compound 2600

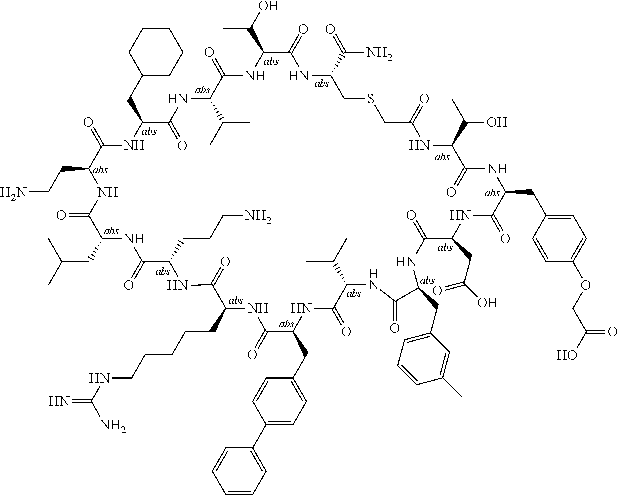 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCCNC(=N)N)NC(=O)[C@H](Cc3ccc(-c4ccccc4)cc3)NC(=O)[C@H](C(C)C)NC2=O)c1]

Compound 2600 was prepared on a 50 μmol scale. The yield of the product was 9.2 mg, and its estimated purity by LCMS analysis was 97.7%. Analysis condition: Retention time=1.72 min; ESI-MS(+) m/z [M+2H]²⁺: 960.3.

### Preparation of Compound 2601

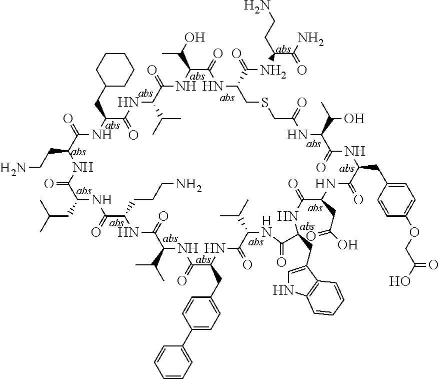 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2601 was prepared on a 50 μmol scale. The yield of the product was 19.8 mg, and its estimated purity by LCMS analysis was 95.6%. Analysis condition: Retention time=1.58 min; ESI-MS(+) m/z [M+H]⁺: 1987.2.

### Preparation of Compound 2602

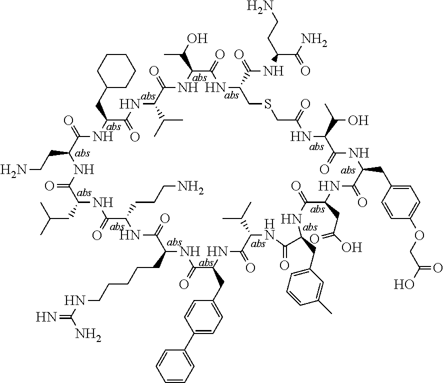 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCCCNC(=N)N)NC(=O)[C@H](Cc3ccc(-c4ccccc4)cc3)NC(=O)[C@H](C(C)C)NC2=O)c1]

Compound 2602 was prepared on a 50 μmol scale. The yield of the product was 31.6 mg, and its estimated purity by LCMS analysis was 97.7%. Analysis condition: Retention time=1.64 min; ESI-MS(+) m/z [M+3H]³⁺: 674.1.

### Preparation of Compound 2603

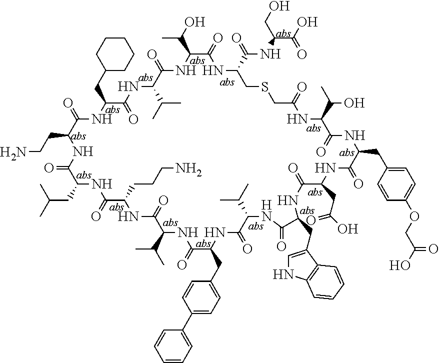 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CO)C(=O)O)NC(=O)[C@H](C(C)O)NC(=O)[C@H](C(C)C)NC(=O)[C@H](CC2CCCCC2)NC(=O)[C@H](CCN)NC1=O]

Compound 2603 was prepared on a 50 μmol scale. The yield of the product was 14.4 mg, and its estimated purity by LCMS analysis was 92.1%. Analysis condition: Retention time=1.65 min; ESI-MS(+) m/z [M+H]⁺: 1975.1.

### Preparation of Compound 2604

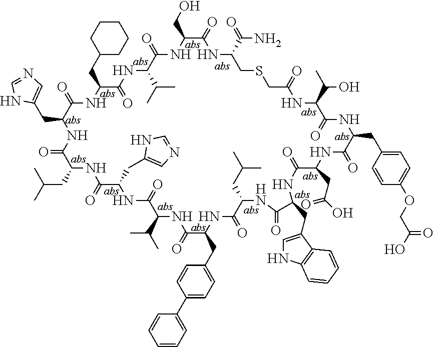 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](CO)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](Cc2cnc[nH]2)NC(=O)[C@H](C(C)C)NC(=O)[C@H](Cc2ccc(-c3ccccc3)cc2)NC1=O]

Compound 2604 was prepared on a 50 μmol scale. The yield of the product was 5.2 mg, and its estimated purity by LCMS analysis was 86.4%. Analysis condition: Retention time=1.72 min; ESI-MS(+) m/z [M+H]⁺: 1946.

### Preparation of Compound 2605

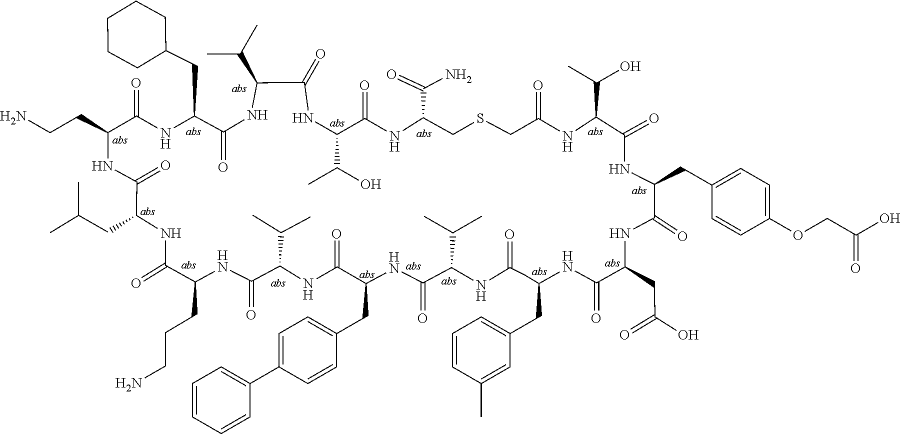

Compound 2605 was prepared on a 50 μmol scale. The yield of the product was 11.8 mg, and its estimated purity by LCMS analysis was 94.1%. Analysis condition A: Retention time=1.68 min; ESI-MS(+) m/z [M+2H]²⁺: 931.2.

### Preparation of Compound 2606

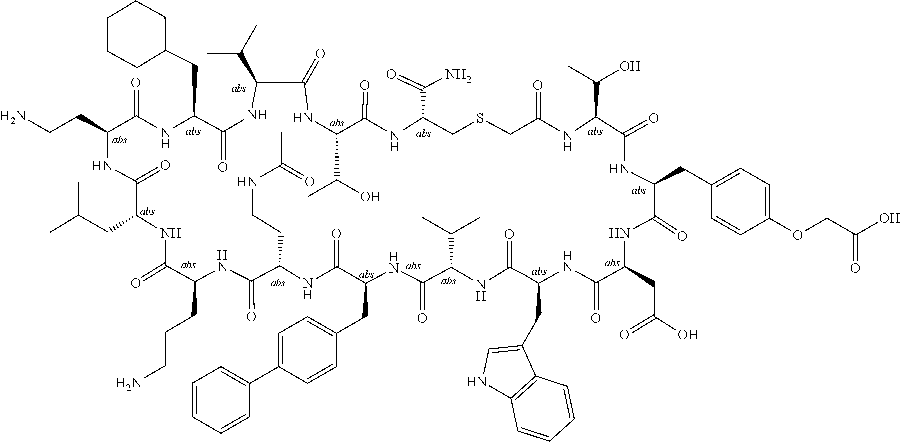

Compound 2606 was prepared on a 50 μmol scale. The yield of the product was 42.6 mg, and its estimated purity by LCMS analysis was 97.3%. Analysis condition A: Retention time=1.57 min; ESI-MS(+) m/z [M+H]⁺: 1929.2.

### Preparation of Compound 2607

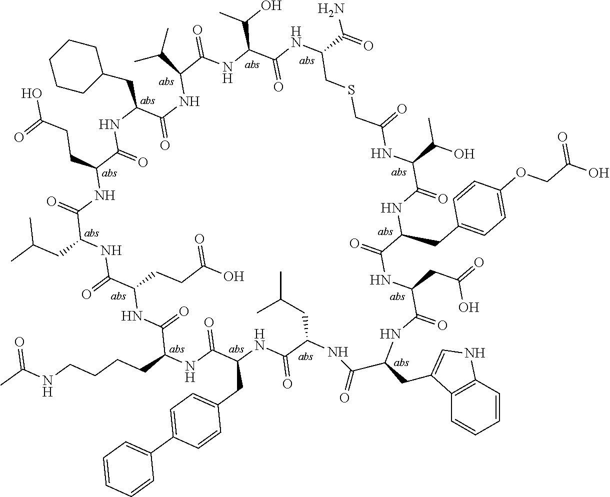 [SMILES: CC(=O)NCCCC[C@@H]1NC(=O)[C@H](Cc2ccc(-c3ccccc3)cc2)NC(=O)[C@H](CC(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](C(C)O)NC(=O)[C@H](C(C)C)NC(=O)[C@H](CC2CCCCC2)NC(=O)[C@H](CCC(=O)O)NC(=O)[C@@H](CC(C)C)NC(=O)[C@H](CCC(=O)O)NC1=O]

Compound 2607 was prepared on a 50 μmol scale. The yield of the product was 14.6 mg, and its estimated purity by LCMS analysis was 97.8%. Analysis condition B: Retention time=1.83 min; ESI-MS(+) m/z [M+2H]²⁺: 1008.2.

### Preparation of Compound 2608

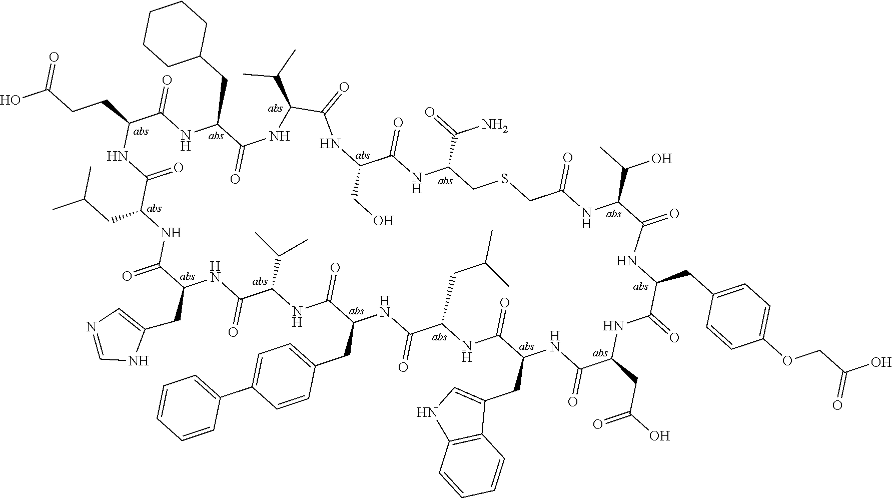 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](CO)NC(=O)[C@H](C(C)C)NC(=O)[C@H](CC2CCCCC2)NC(=O)[C@H](CCC(=O)O)NC(=O)[C@@H](CC(C)C)NC(=O)[C@H](Cc2cnc[nH]2)NC(=O)[C@H](C(C)C)NC(=O)[C@H](Cc2ccc(-c3ccccc3)cc2)NC1=O]

Compound 2608 was prepared on a 50 μmol scale. The yield of the product was 5.7 mg, and its estimated purity by LCMS analysis was 96.5%. Analysis condition B: Retention time=1.77 min; ESI-MS(+) m/z [M+2H]²⁺: 970.2.

### Preparation of Compound 2609

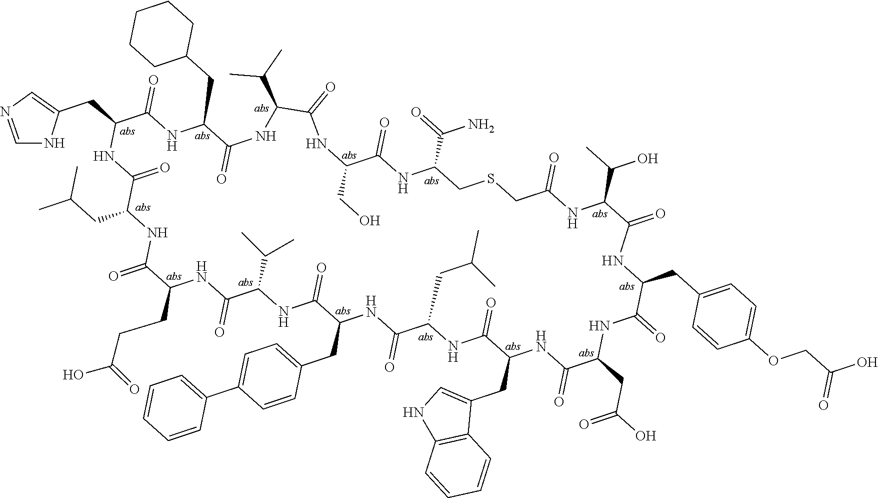 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](CO)NC(=O)[C@H](C(C)C)NC(=O)[C@H](CC2CCCCC2)NC(=O)[C@H](Cc2cnc[nH]2)NC(=O)[C@@H](CC(C)C)NC(=O)[C@H](CCC(=O)O)NC(=O)[C@H](C(C)C)NC(=O)[C@H](Cc2ccc(-c3ccccc3)cc2)NC1=O]

Compound 2609 was prepared on a 50 μmol scale. The yield of the product was 18.8 mg, and its estimated purity by LCMS analysis was 95.5%. Analysis condition B: Retention time=1.8 min; ESI-MS(+) m/z [M+H]⁺: 1936.

### Preparation of Compound 2610

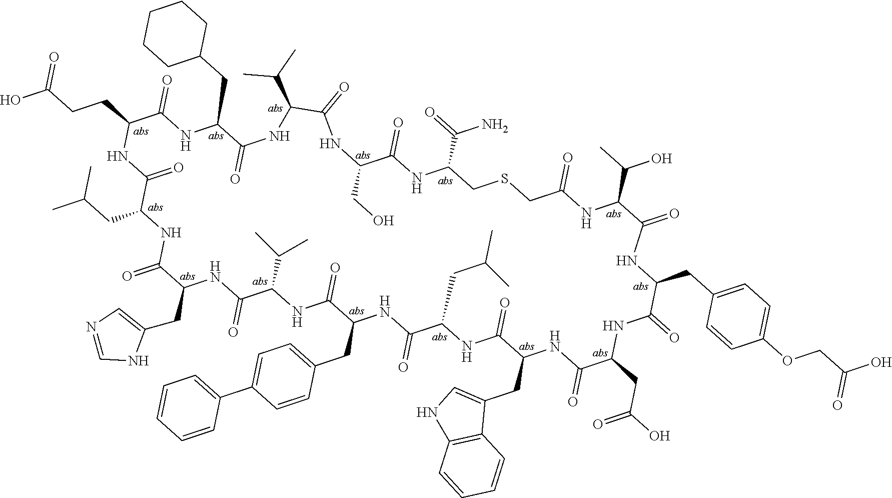 [SMILES: CC(C)C[C@@H]1NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(N)=O)NC(=O)[C@H](CO)NC(=O)[C@H](C(C)C)NC(=O)[C@H](CC2CCCCC2)NC(=O)[C@H](CCC(=O)O)NC(=O)[C@@H](CC(C)C)NC(=O)[C@H](Cc2cnc[nH]2)NC(=O)[C@H](C(C)C)NC(=O)[C@H](Cc2ccc(-c3ccccc3)cc2)NC1=O]

Compound 2610 was prepared on a 50 μmol scale. The yield of the product was 5.3 mg, and its estimated purity by LCMS analysis was 96.3%. Analysis condition A: Retention time=1.51 min; ESI-MS(+) m/z [M+H]⁺: 1938.2.

### Preparation of Compound 2611

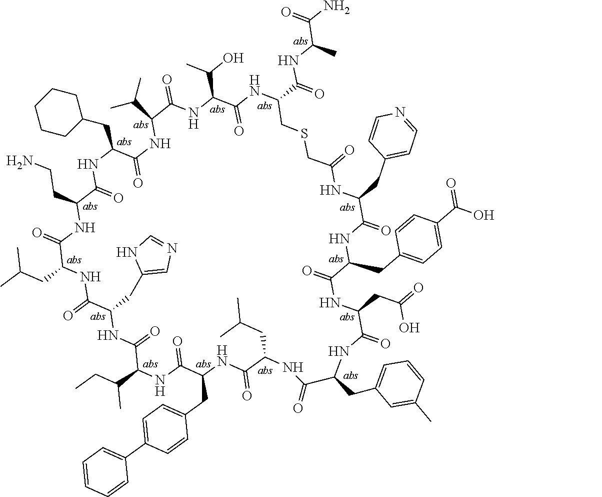 [SMILES: CCC(C)[C@@H]1NC(=O)[C@H](Cc2ccc(-c3ccccc3)cc2)NC(=O)[C@H](CC(C)C)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](C)C(N)=O)NC(=O)[C@H](C(C)O)NC(=O)[C@H](C(C)C)NC(=O)[C@H](CC2CCCCC2)NC(=O)[C@H](CCN)NC(=O)[C@@H](CC(C)C)NC(=O)[C@H](Cc2cnc[nH]2)NC1=O]

Compound 2611 was prepared on a 50 μmol scale. The yield of the product was 33.6 mg, and its estimated purity by LCMS analysis was 95.2%. Analysis condition B: Retention time=1.62 min; ESI-MS(+) m/z [M+2H]²⁺: 1001.2.

### Preparation of Compound 2612

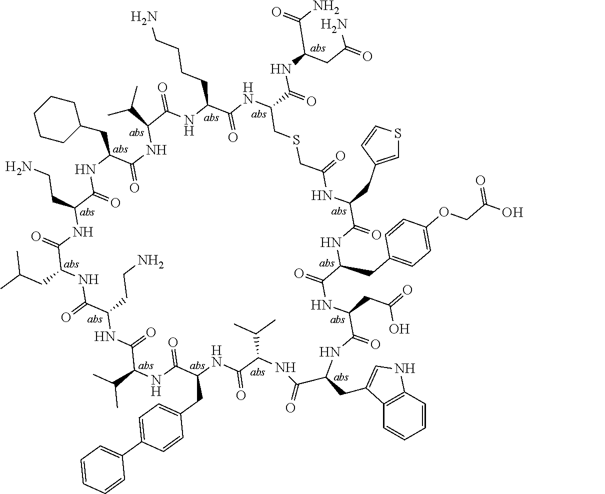 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@H](CC(N)=O)C(N)=O)NC(=O)[C@H](CCCCN)NC(=O)[C@H](C(C)C)NC(=O)[C@H](CC2CCCCC2)NC(=O)[C@H](CCN)NC1=O]

Compound 2612 was prepared on a 50 μmol scale. The yield of the product was 25 mg, and its estimated purity by LCMS analysis was 97.4%. Analysis condition B: Retention time=1.63 min; ESI-MS(+) m/z [M+2H]²⁺: 1033.1.

### Preparation of Compound 2613

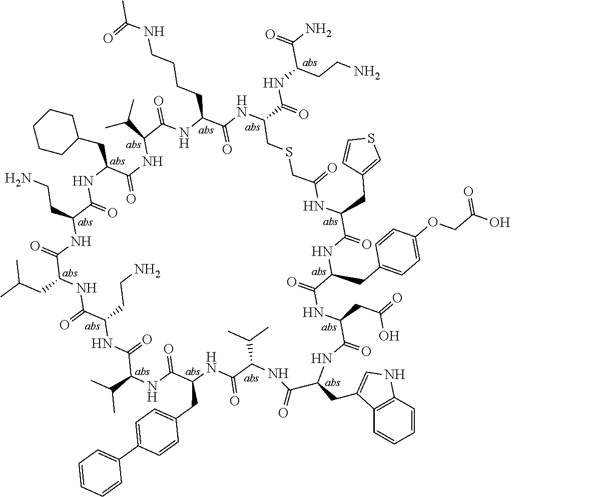 [SMILES: CC(=O)NCCCC[C@@H]1NC(=O)[C@H](C(C)C)NC(=O)[C@H](CC2CCCCC2)NC(=O)[C@H](CCN)NC(=O)[C@@H](CC(C)C)NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(=O)N[C@@H](CCN)C(N)=O)NC1=O]

Compound 2613 was prepared on a 50 μmol scale. The yield of the product was 11.6 mg, and its estimated purity by LCMS analysis was 97%. Analysis condition B: Retention time=1.67 min; ESI-MS(+) m/z [M+3H]³⁺: 699.

### Preparation of Compound 2614

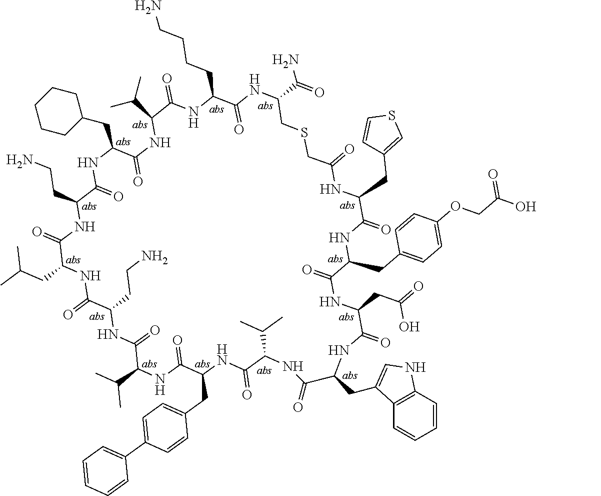 [SMILES: CC(C)C[C@H]1NC(=O)[C@H](CCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccsc2)NC(=O)CSC[C@@H](C(N)=O)NC(=O)[C@H](CCCCN)NC(=O)[C@H](C(C)C)NC(=O)[C@H](CC2CCCCC2)NC(=O)[C@H](CCN)NC1=O]

Compound 2614 was prepared on a 50 μmol scale. The yield of the product was 23.9 mg, and its estimated purity by LCMS analysis was 98.8%. Analysis condition B: Retention time=1.65 min; ESI-MS(+) m/z [M+H]⁺: 1952.

### Preparation of Compound 2615

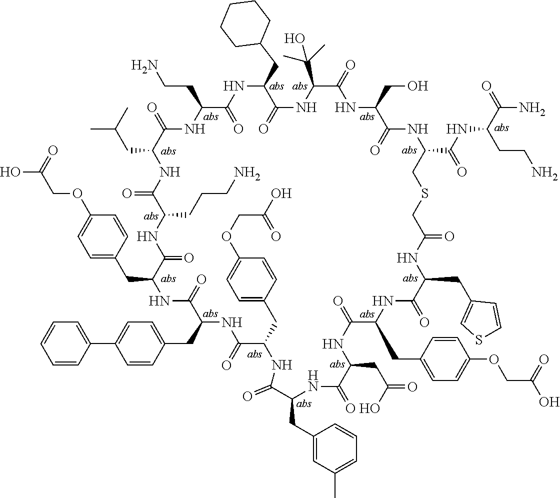 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccsc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2615 was prepared on a 50 μmol scale. The yield of the product was 16.4 mg, and its estimated purity by LCMS analysis was 98.3%. Analysis condition B: Retention time=1.67 min; ESI-MS(+) m/z [M+3H]³⁺: 754.

### Preparation of Compound 2616

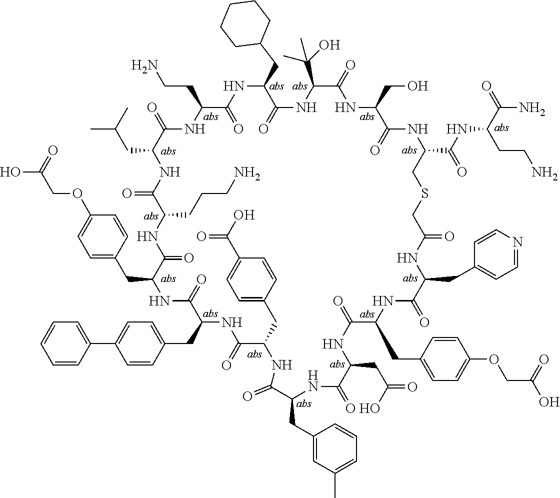 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2616 was prepared on a 50 μmol scale. The yield of the product was 5.1 mg, and its estimated purity by LCMS analysis was 95.2%. Analysis condition: Retention time=1.58 min; ESI-MS(+) m/z [M+2H]²⁺: 1113.1.

### Preparation of Compound 2617

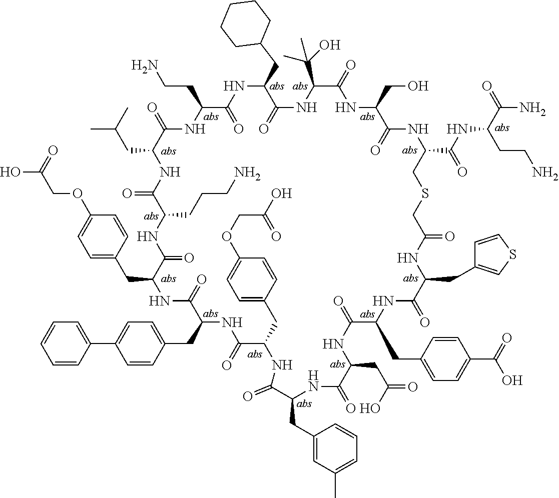 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2617 was prepared on a 50 μmol scale. The yield of the product was 29.9 mg, and its estimated purity by LCMS analysis was 95.9%. Analysis condition: Retention time=1.68 min; ESI-MS(+) m/z [M+3H]³⁺: 744.

### Preparation of Compound 2618

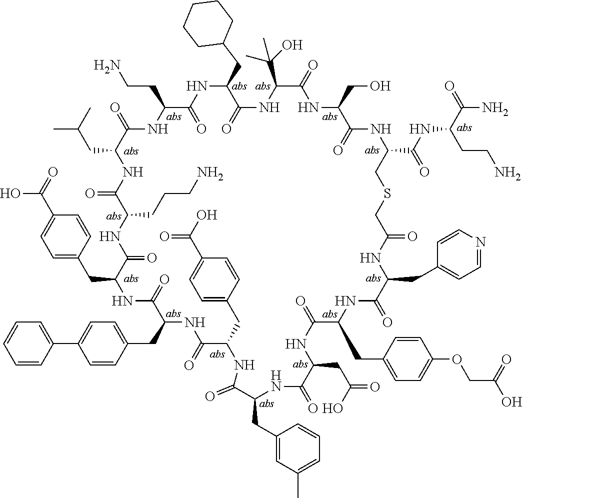 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2618 was prepared on a 50 μmol scale. The yield of the product was 3.6 mg, and its estimated purity by LCMS analysis was 100%. Analysis condition A: Retention time=1.39 min; ESI-MS(+) m/z [M+2H]²⁺: 1098.2.

### Preparation of Compound 2619

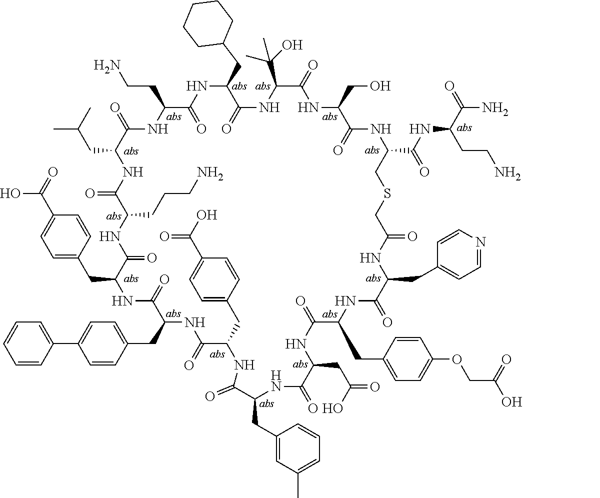 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2619 was prepared on a 50 μmol scale. The yield of the product was 30.4 mg, and its estimated purity by LCMS analysis was 95.6%. Analysis condition: Retention time=1.43 min; ESI-MS(+) m/z [M+2H]²⁺: 1105.2.

### Preparation of Compound 2620

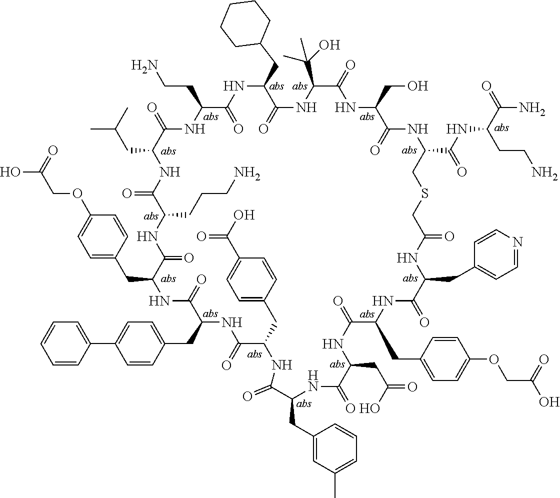 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2620 was prepared on a 50 μmol scale. The yield of the product was 10.6 mg, and its estimated purity by LCMS analysis was 96.9%. Analysis condition A: Retention time=1.45 min; ESI-MS(+) m/z [M+2H]²⁺: 1112.9.

### Preparation of Compound 2621

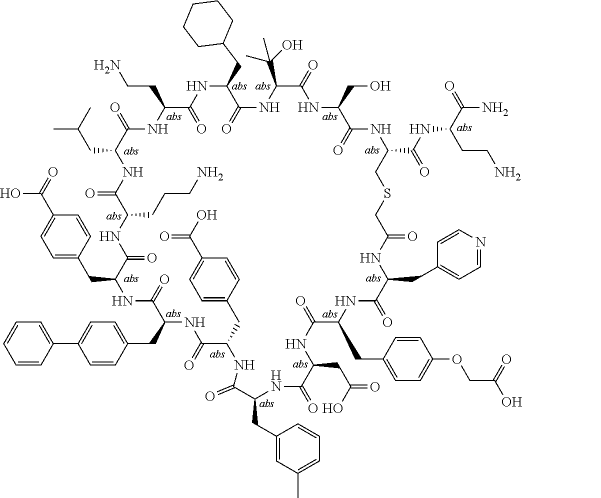 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2621 was prepared on a 50 μmol scale. The yield of the product was 11.7 mg, and its estimated purity by LCMS analysis was 95.6%. Analysis condition: Retention time=1.58 min; ESI-MS(+) m/z [M+3H]³⁺: 732.2.

### Preparation of Compound 2622

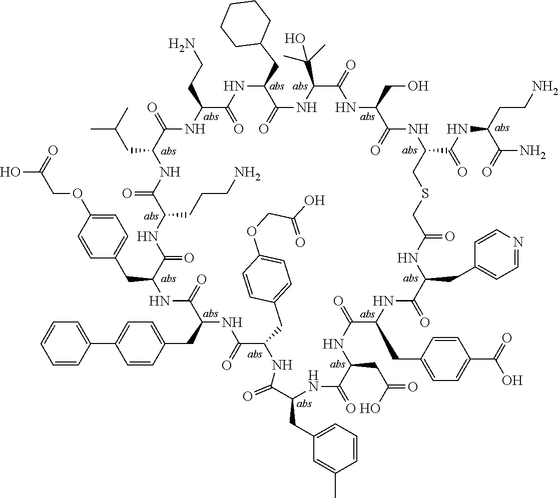 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2622 was prepared on a 50 μmol scale. The yield of the product was 17.7 mg, and its estimated purity by LCMS analysis was 96%. Analysis condition B: Retention time=1.51 min; ESI-MS(+) m/z [M+3H]³⁺: 742.2.

### Preparation of Compound 2623

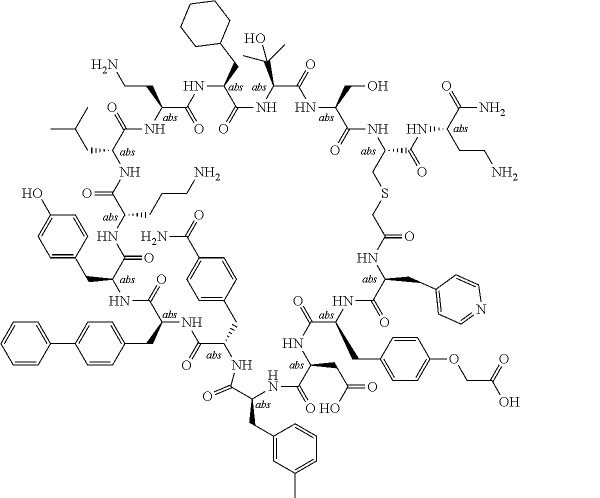 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2623 was prepared on a 50 μmol scale. The yield of the product was 21.9 mg, and its estimated purity by LCMS analysis was 98.5%. Analysis condition A: Retention time=1.54 min; ESI-MS(+) m/z [M+2H]²⁺: 1083.2.

### Preparation of Compound 2624

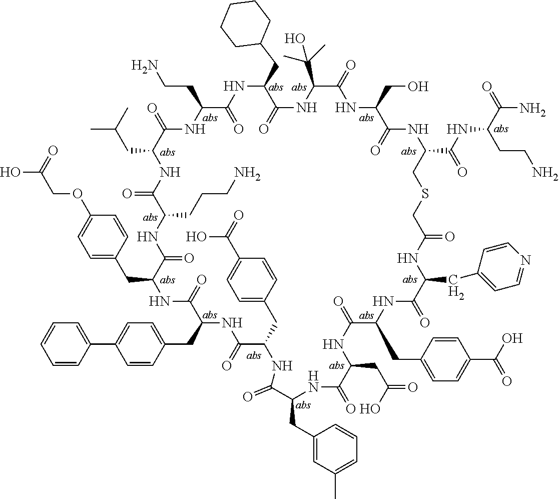 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2624 was prepared on a 50 μmol scale. The yield of the product was 13.5 mg, and its estimated purity by LCMS analysis was 96.3%. Analysis condition: Retention time=1.57 min; ESI-MS(+) m/z [M+3H]³⁺: 732.

### Preparation of Compound 2625

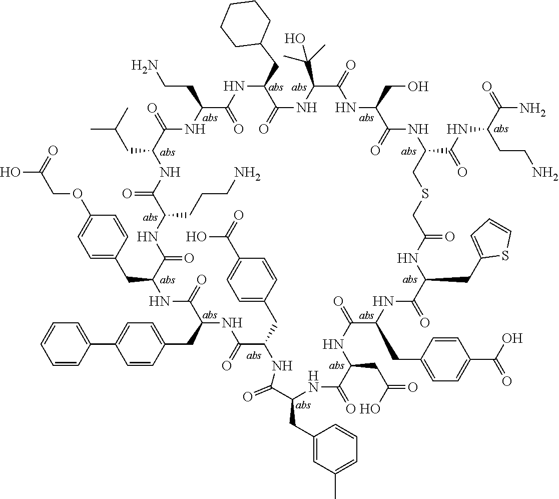 [SMILES: Cc1cccc(C[C@@H]2NC(=O)[C@H](CC(=O)O)NC(=O)[C@H](Cc3ccc(C(=O)O)cc3)NC(=O)[C@H](Cc3cccs3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2625 was prepared on a 30 μmol scale. The yield of the product was 15.5 mg, and its estimated purity by LCMS analysis was 96.5%. Analysis condition: Retention time=1.69 min; ESI-MS(+) m/z [M+2H]²⁺: 1100.2.

### Preparation of Compound 2626

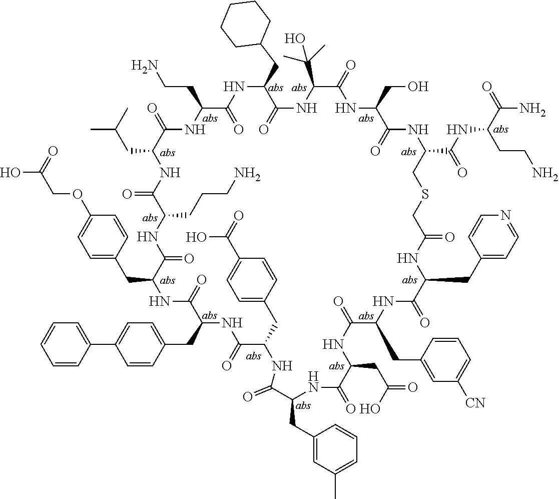 [SMILES: Cc1cccc(C[C@@H]2NC(=O)[C@H](CC(=O)O)NC(=O)[C@H](Cc3cccc(C#N)c3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2626 was prepared on a 50 μmol scale. The yield of the product was 20 mg, and its estimated purity by LCMS analysis was 95.1%. Analysis condition: Retention time=1.68 min; ESI-MS(+) m/z [M+3H]³⁺: 726.1.

### Preparation of Compound 2627

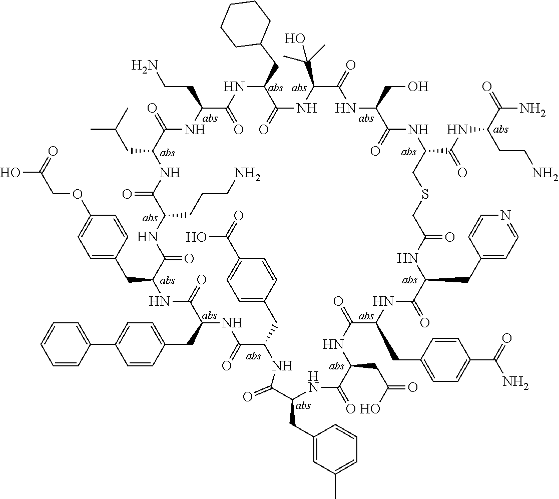 [SMILES: Cc1cccc(C[C@@H]2NC(=O)[C@H](CC(=O)O)NC(=O)[C@H](Cc3ccc(C(N)=O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2627 was prepared on a 50 μmol scale. The yield of the product was 9.3 mg, and its estimated purity by LCMS analysis was 96.5%. Analysis condition: Retention time=1.6 min; ESI-MS(+) m/z [M+3H]³⁺: 732.2.

### Preparation of Compound 2628

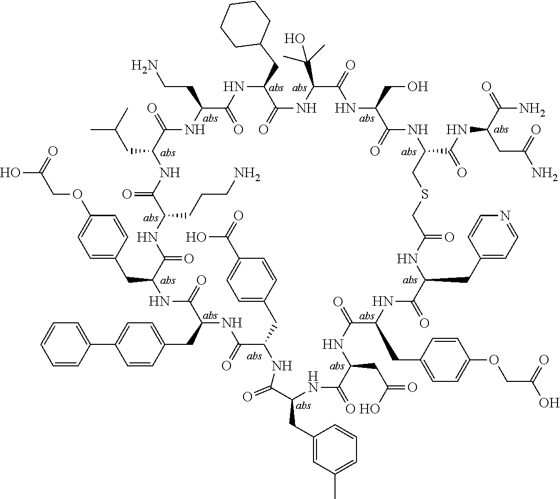 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2628 was prepared on a 50 μmol scale. The yield of the product was 9.2 mg, and its estimated purity by LCMS analysis was 100%. Analysis condition A: Retention time=1.42 min; ESI-MS(+) m/z [M+2H]²⁺: 1120.1.

### Preparation of Compound 2629

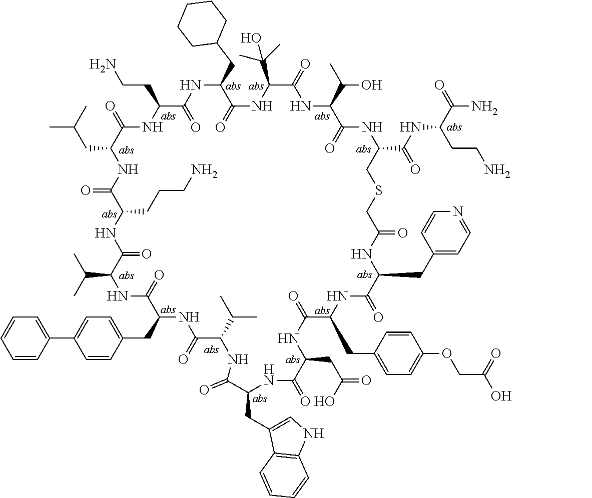 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccncc2)NC(=O)CSC[C@@H](C(=O)N[C@@H](CCN)C(N)=O)NC(=O)[C@H](C(C)O)NC(=O)[C@H](C(C)(C)O)NC(=O)[C@H](CC2CCCCC2)NC(=O)[C@H](CCN)NC1=O]

Compound 2629 was prepared on a 40 μmol scale. The yield of the product was 18.4 mg, and its estimated purity by LCMS analysis was 95.2%. Analysis condition A: Retention time=1.62 min; ESI-MS(+) m/z [M+2H]²⁺: 1025.2.

### Preparation of Compound 2630

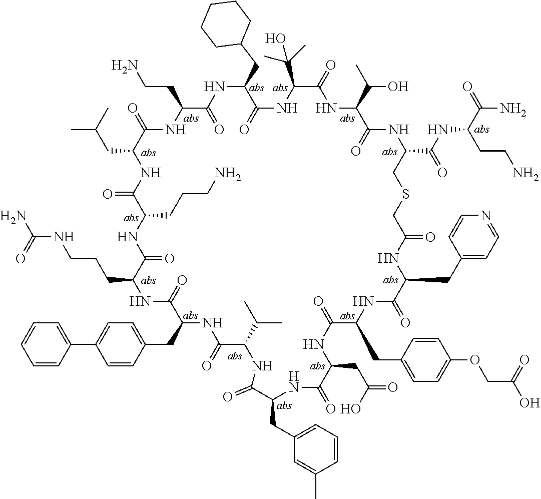 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2630 was prepared on a 40 μmol scale. The yield of the product was 30.1 mg, and its estimated purity by LCMS analysis was 95.2%. Analysis condition A: Retention time=1.57 min; ESI-MS(+) m/z [M+2H]²⁺: 1042.1.

### Preparation of Compound 2631

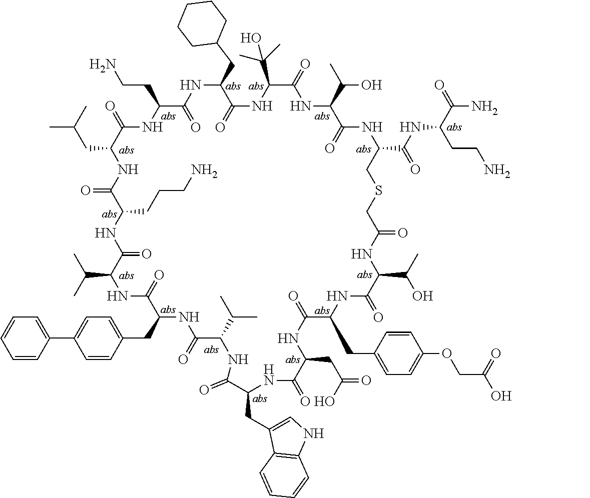 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](C(C)O)NC(=O)CSC[C@@H](C(=O)N[C@@H](CCN)C(N)=O)NC(=O)[C@H](C(C)O)NC(=O)[C@H](C(C)(C)O)NC(=O)[C@H](CC2CCCCC2)NC(=O)[C@H](CCN)NC1=O]

Compound 2631 was prepared on a 40 μmol scale. The yield of the product was 26.2 mg, and its estimated purity by LCMS analysis was 95.8%. Analysis condition: Retention time=1.66 min; ESI-MS(+) m/z [M+2H]²⁺: 1002.2.

### Preparation of Compound 2632

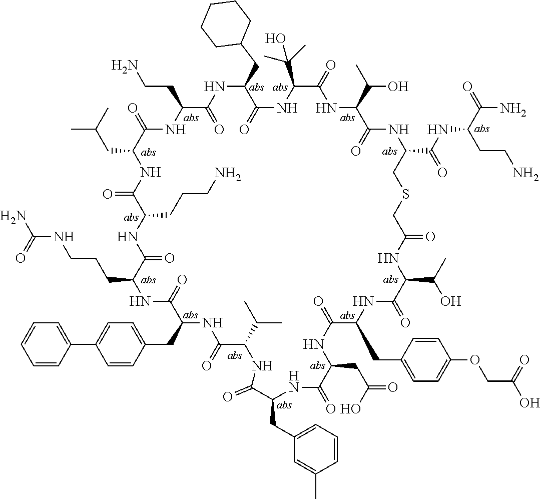 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2632 was prepared on a 40 μmol scale. The yield of the product was 12.2 mg, and its estimated purity by LCMS analysis was 96.1%. Analysis condition A: Retention time=1.56 min; ESI-MS(+) m/z [M+3H]³⁺: 679.2.

### Preparation of Compound 2633

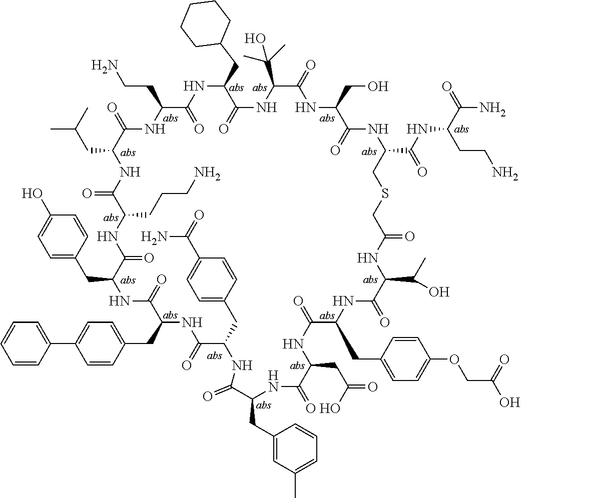 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2633 was prepared on a 50 μmol scale. The yield of the product was 21 mg, and its estimated purity by LCMS analysis was 96.3%. Analysis condition B: Retention time=1.55 min; ESI-MS(+) m/z [M+2H]²⁺: 1059.9.

### Preparation of Compound 2634

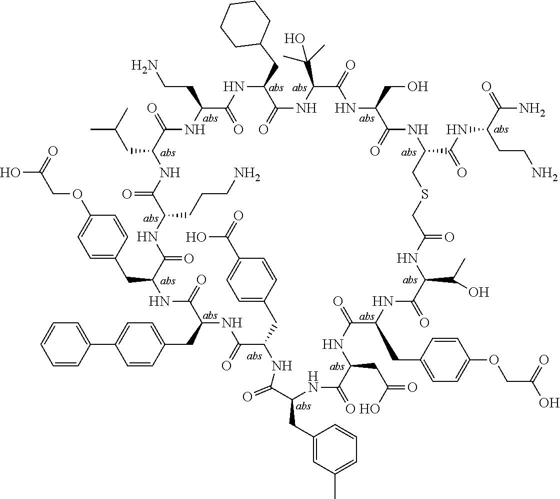 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2634 was prepared on a 50 μmol scale. The yield of the product was 21.7 mg, and its estimated purity by LCMS analysis was 97.6%. Analysis condition B: Retention time=1.56 min; ESI-MS(+) m/z [M+2H]²⁺: 1089.2.

### Preparation of Compound 2635

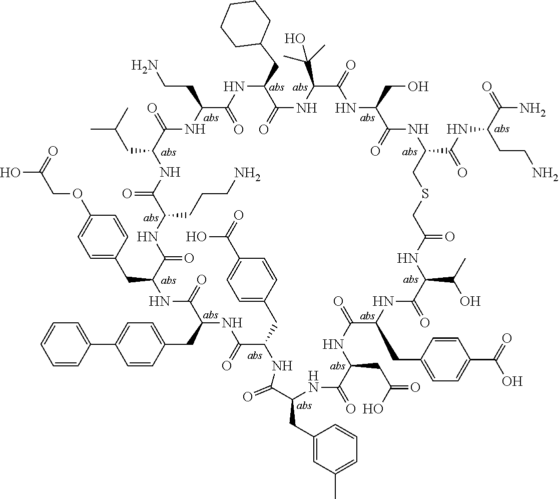 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2635 was prepared on a 50 μmol scale. The yield of the product was 12.5 mg, and its estimated purity by LCMS analysis was 100%. Analysis condition A: Retention time=1.38 min; ESI-MS(+) m/z [M+2H]²⁺: 1074.2.

### Preparation of Compound 2636

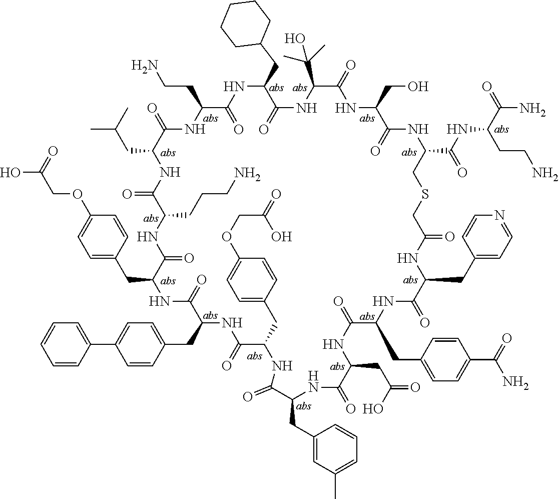 [SMILES: Cc1cccc(C[C@@H]2NC(=O)[C@H](CC(=O)O)NC(=O)[C@H](Cc3ccc(C(N)=O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2636 was prepared on a 50 μmol scale. The yield of the product was 44.9 mg, and its estimated purity by LCMS analysis was 95.3%. Analysis condition: Retention time=1.53 min; ESI-MS(+) m/z [M+3H]³⁺: 742.2.

### Preparation of Compound 2637

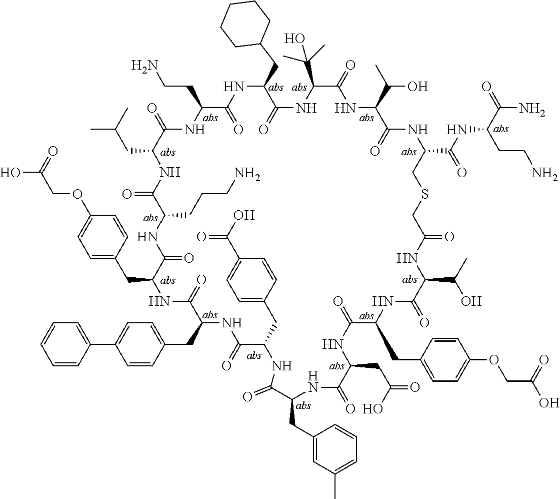 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCN)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(C(=O)O)cc3)NC2=O)c1]

Compound 2637 was prepared on a 50 μmol scale. The yield of the product was 44.7 mg, and its estimated purity by LCMS analysis was 98.8%. Analysis condition: Retention time=1.44 min; ESI-MS(+) m/z [M+2H]²⁺: 1081.2.

### Preparation of Compound 2638

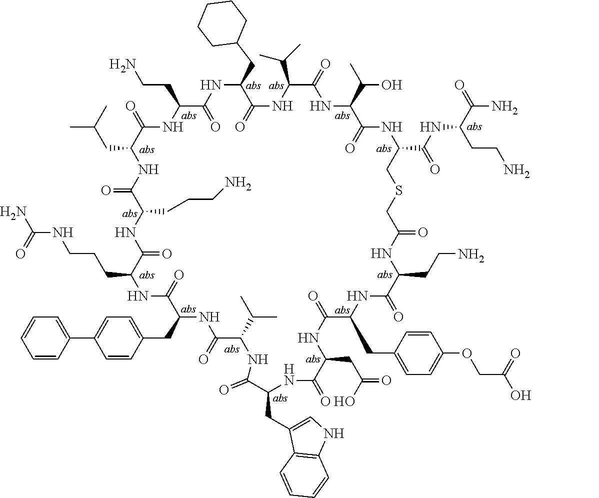 [SMILES: CC(C)C[C@H]1NC(=O)[C@H](CCCN)NC(=O)[C@H](CCCNC(N)=O)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](CCN)NC(=O)CSC[C@@H](C(=O)N[C@@H](CCN)C(N)=O)NC(=O)[C@H](C(C)O)NC(=O)[C@H](C(C)C)NC(=O)[C@H](CC2CCCCC2)NC(=O)[C@H](CCN)NC1=O]

Compound 2638 was prepared on a 50 μmol scale. The yield of the product was 31.8 mg, and its estimated purity by LCMS analysis was 95.4%. Analysis condition B: Retention time=1.44 min; ESI-MS(+) m/z [M+2H]²⁺: 1022.2.

### Preparation of Compound 2639

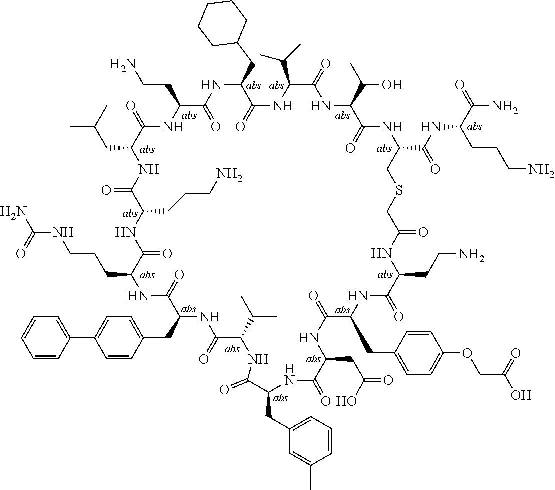 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCCN)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2639 was prepared on a 50 μmol scale. The yield of the product was 6.7 mg, and its estimated purity by LCMS analysis was 98.4%. Analysis condition B: Retention time=1.51 min; ESI-MS(+) m/z [M+2H]²⁺: 1017.1.

### Preparation of Compound 2640

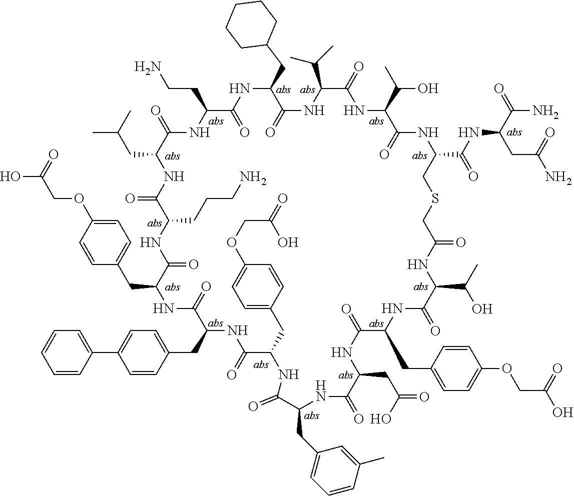 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2640 was prepared on a 50 μmol scale. The yield of the product was 38.9 mg, and its estimated purity by LCMS analysis was 95.7%. Analysis condition: Retention time=1.43 min; ESI-MS(+) m/z [M+2H]²⁺: 1110.3.

### Preparation of Compound 2641

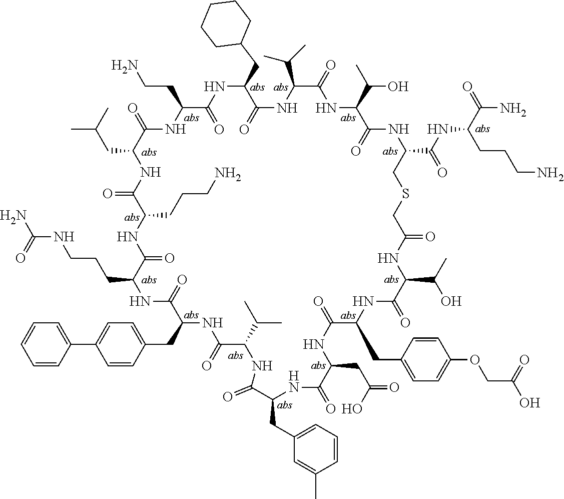 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CCCN)C(N)=O)NC(=O)[C@H](C(C)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCNC(N)=O)NC(=O)[C@H](Cc3ccc(-c4ccccc4)cc3)NC(=O)[C@H](C(C)C)NC2=O)c1]

Compound 2641 was prepared on a 50 μmol scale. The yield of the product was 18.1 mg, and its estimated purity by LCMS analysis was 97.2%. Analysis condition B: Retention time=1.55 min; ESI-MS(+) m/z [M+2H]²⁺: 673.7.

### Preparation of Compound 2642

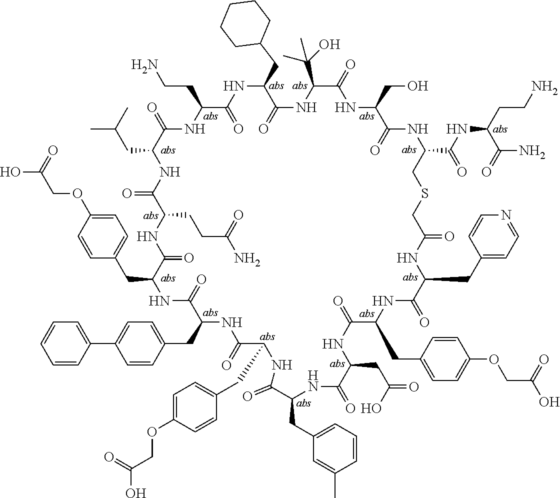 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2642 was prepared on a 50 μmol scale. The yield of the product was 26.6 mg, and its estimated purity by LCMS analysis was 98.7%. Analysis condition B: Retention time=1.6 min; ESI-MS(+) m/z [M+2H]²⁺: 1135.1.

### Preparation of Compound 2643

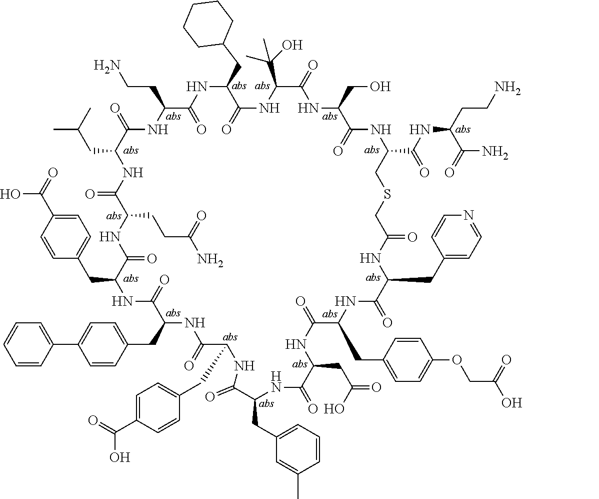 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCC(N)=O)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2643 was prepared on a 50 μmol scale. The yield of the product was 57.2 mg, and its estimated purity by LCMS analysis was 95.6%. Analysis condition: Retention time=1.42, 1.47 min; ESI-MS(+) m/z [M+2H]²⁺: 1104.94, 1105.07.

### Preparation of Compound 2644

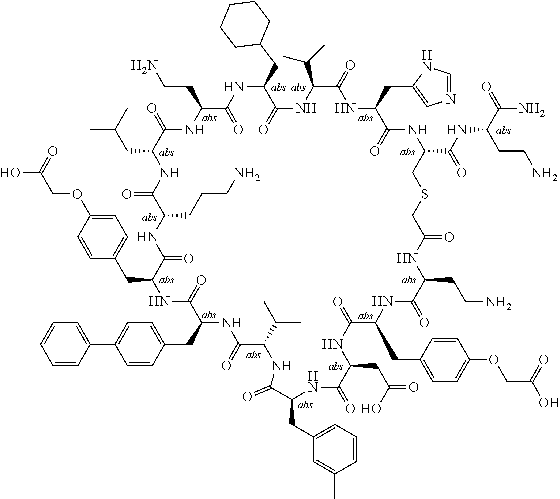 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](Cc3cnc[nH]3)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](C(C)C)NC2=O)c1]

Compound 2644 was prepared on a 50 μmol scale. The yield of the product was 9 mg, and its estimated purity by LCMS analysis was 99.4%. Analysis condition A: Retention time=1.55 min; ESI-MS(+) m/z [M+2H]²⁺: 1060.2.

### Preparation of Compound 2645

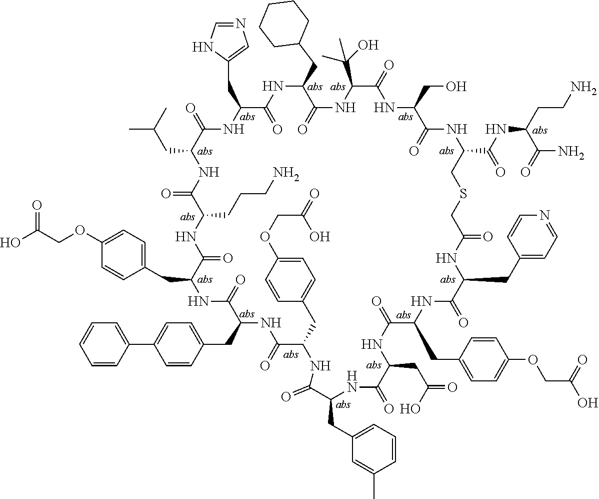 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2645 was prepared on a 30 μmol scale. The yield of the product was 6.4 mg, and its estimated purity by LCMS analysis was 95%. Analysis condition B: Retention time=1.51 min; ESI-MS(+) m/z [M+3H]³⁺: 765.

### Preparation of Compound 2646

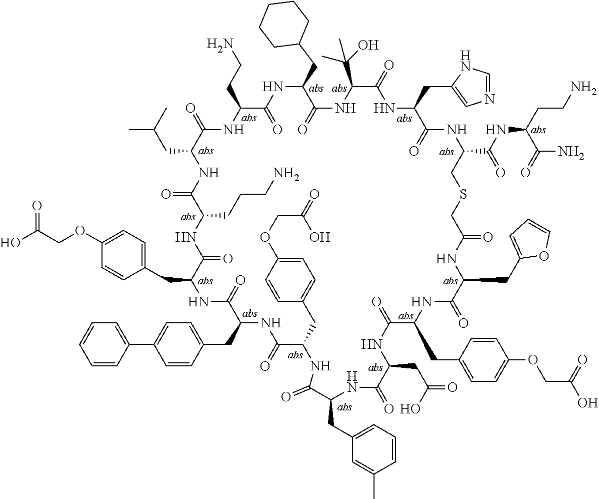 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccco3)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2646 was prepared on a 30 μmol scale. The yield of the product was 2.8 mg, and its estimated purity by LCMS analysis was 94.7%. Analysis condition A: Retention time=1.47 min; ESI-MS(+) m/z [M+2H]²⁺: 1147.1.

### Preparation of Compound 2647

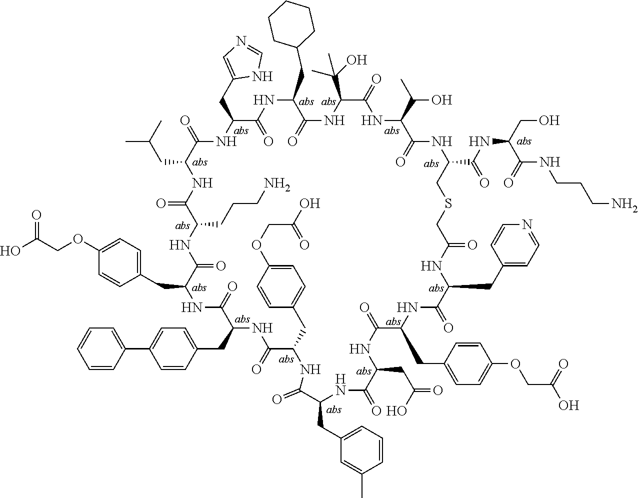 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CO)C(=O)NCCCN)NC(=O)[C@H](C(C)O)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2647 was prepared on a 30 μmol scale. The yield of the product was 4.6 mg, and its estimated purity by LCMS analysis was 95.1%. Analysis condition B: Retention time=1.57 min; ESI-MS(+) m/z [M+3H]³⁺: 784.1.

### Preparation of Compound 2648

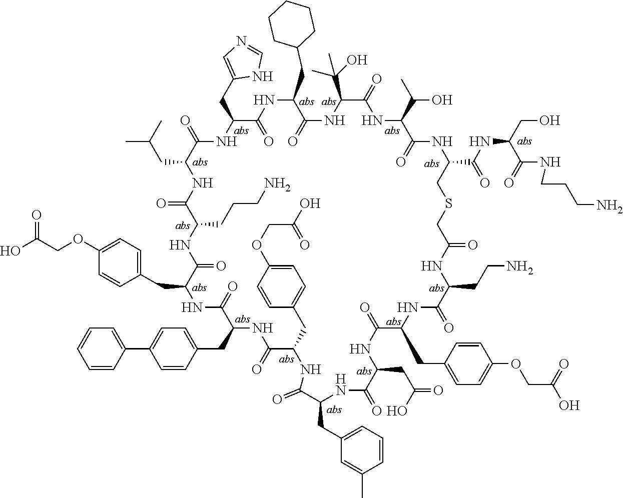 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CO)C(=O)NCCCN)NC(=O)[C@H](C(C)O)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2648 was prepared on a 30 μmol scale. The yield of the product was 6.8 mg, and its estimated purity by LCMS analysis was 96.6%. Analysis condition A: Retention time=1.56 min; ESI-MS(+) m/z [M+3H]³⁺: 768.

### Preparation of Compound 2649

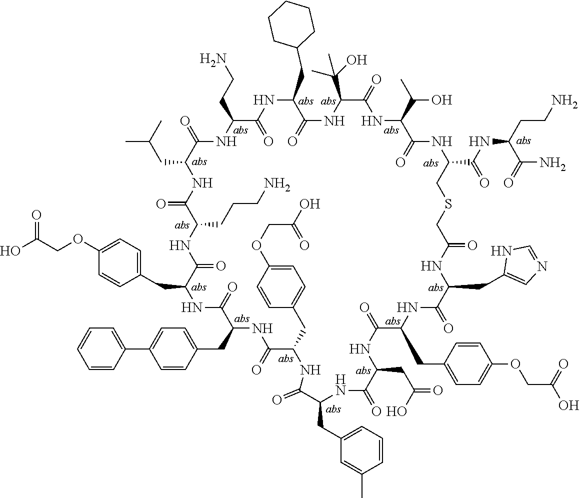 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@@H](CCN)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2649 was prepared on a 50 μmol scale. The yield of the product was 35.1 mg, and its estimated purity by LCMS analysis was 86.1%. Analysis condition A: Retention time=1.48 min; ESI-MS(+) m/z [M+2H]²⁺: 1129.3.

### Preparation of Compound 2650

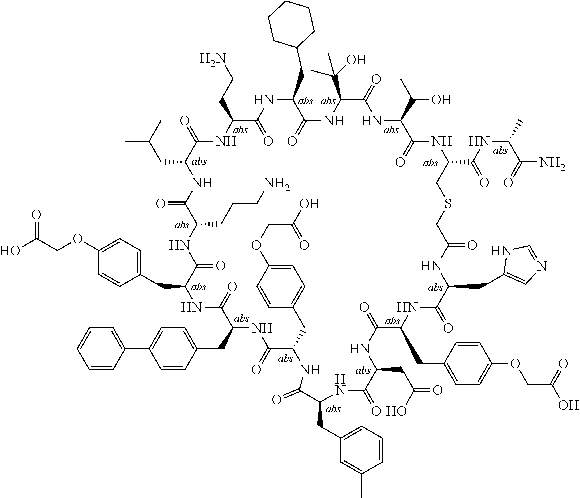 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@H](C)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2650 was prepared on a 50 μmol scale. The yield of the product was 35.7 mg, and its estimated purity by LCMS analysis was 96.3%. Analysis condition A: Retention time=1.48 min; ESI-MS(+) m/z [M+2H]²⁺: 1115.1.

### Preparation of Compound 2651

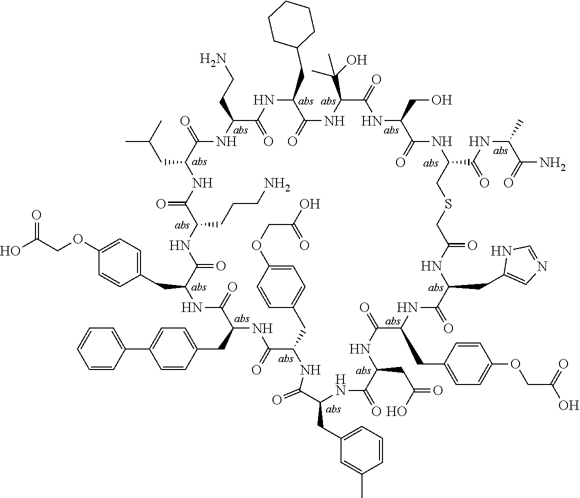 [SMILES: Cc1cccc(C[C@@H]2NC(=O)[C@H](CC(=O)O)NC(=O)[C@H](Cc3ccc(OCC(=O)O)cc3)NC(=O)[C@H](Cc3cnc[nH]3)NC(=O)CSC[C@@H](C(=O)N[C@H](C)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2651 was prepared on a 50 μmol scale. The yield of the product was 20.3 mg, and its estimated purity by LCMS analysis was 100%. Analysis condition A: Retention time=1.46 min; ESI-MS(+) m/z [M+2H]²⁺: 1093.1.

### Preparation of Compound 2652

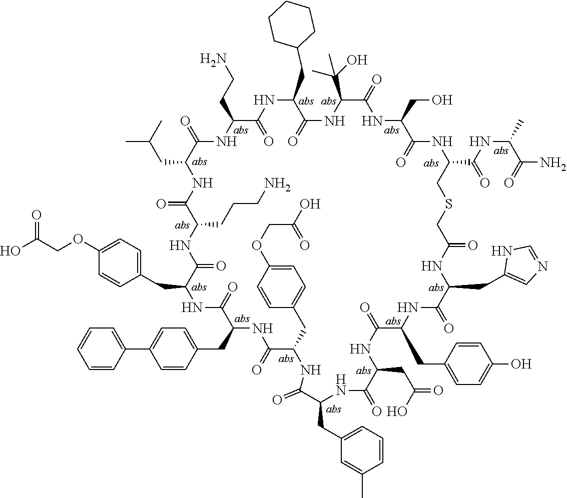 [SMILES: Cc1cccc(C[C@@H]2NC(=O)[C@H](CC(=O)O)NC(=O)[C@H](Cc3ccc(O)cc3)NC(=O)[C@H](Cc3cnc[nH]3)NC(=O)CSC[C@@H](C(=O)N[C@H](C)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2652 was prepared on a 50 μmol scale. The yield of the product was 24.7 mg, and its estimated purity by LCMS analysis was 97.6%. Analysis condition A: Retention time=1.54 min; ESI-MS(+) m/z [M+2H]²⁺: 1078.9.

### Preparation of Compound 2653

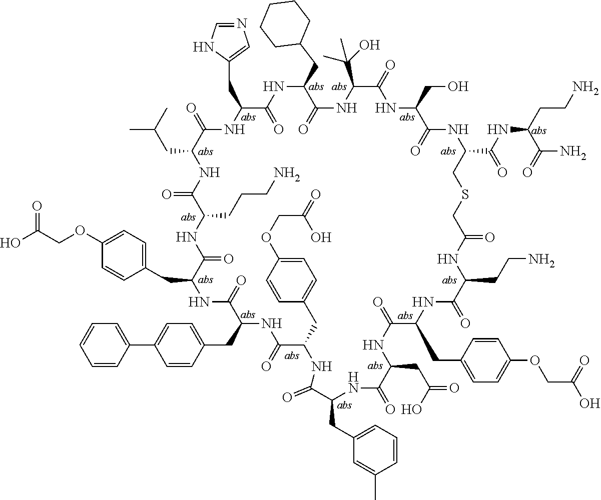 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](Cc3cnc[nH]3)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2653 was prepared on a 30 μmol scale. The yield of the product was 6.1 mg, and its estimated purity by LCMS analysis was 99%. Analysis condition A: Retention time=1.51 min; ESI-MS(+) m/z [M+2H]²⁺: 1122.3.

### Preparation of Compound 2654

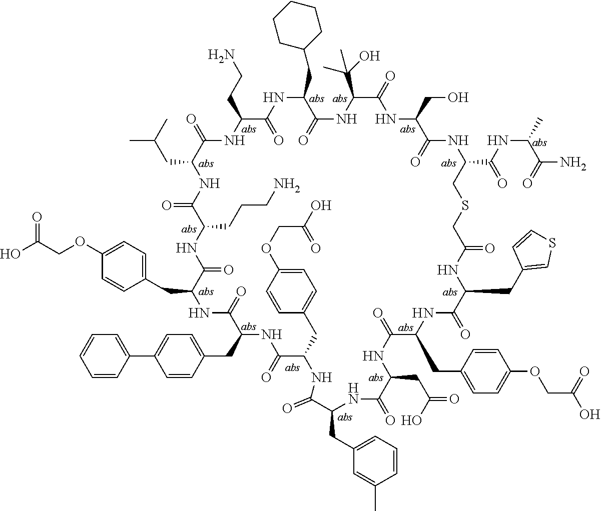 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccsc3)NC(=O)CSC[C@@H](C(=O)N[C@H](C)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2654 was prepared on a 50 μmol scale. The yield of the product was 33.3 mg, and its estimated purity by LCMS analysis was 100%. Analysis condition A: Retention time=1.5 min; ESI-MS(+) m/z [M+2H]²⁺: 1116.1.

### Preparation of Compound 2655

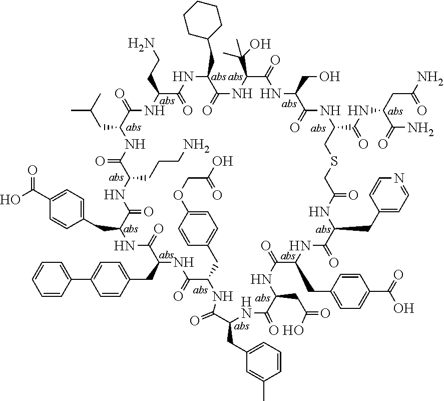 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Compound 2655 was prepared on a 50 μmol scale. The yield of the product was 9.2 mg, and its estimated purity by LCMS analysis was 100%. Analysis condition A: Retention time=1.36 min; ESI-MS(+) m/z [M+2H]²⁺: 1105.1.

### Preparation of Compound 2656

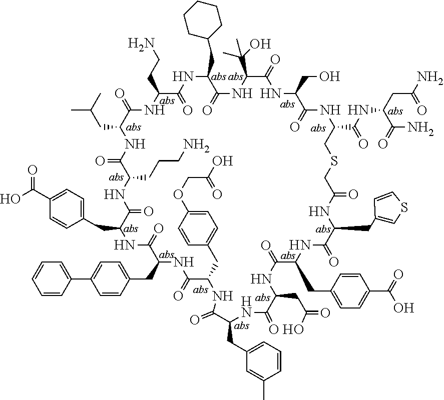 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@H](CC(N)=O)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Compound 2656 was prepared on a 50 μmol scale. The yield of the product was 27.4 mg, and its estimated purity by LCMS analysis was 95.4%. Analysis condition A: Retention time=1.37 min; ESI-MS(+) m/z [M+2H]²⁺: 1107.

### Preparation of Compound 2657

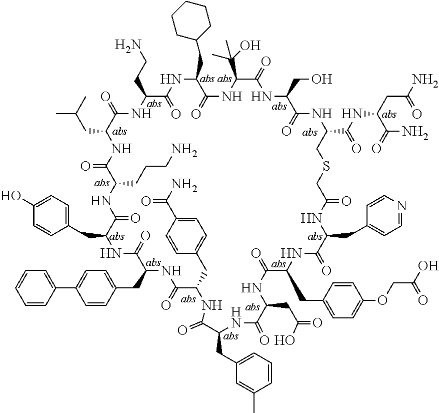 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@H](CC(N)=O)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2657 was prepared on a 50 μmol scale. The yield of the product was 35 mg, and its estimated purity by LCMS analysis was 95.1%. Analysis condition B: Retention time=1.5 min; ESI-MS(+) m/z [M+3H]³⁺: 727.2.

### Preparation of Compound 2658

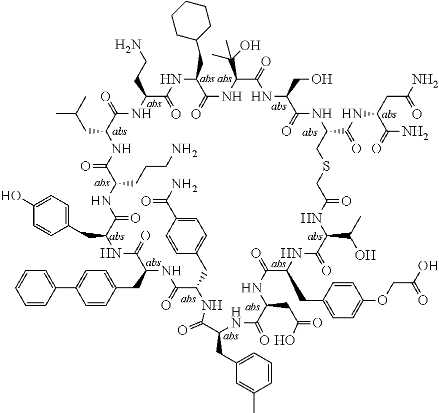 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@H](CC(N)=O)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2658 was prepared on a 50 μmol scale. The yield of the product was 43.6 mg, and its estimated purity by LCMS analysis was 98.9%. Analysis condition B: Retention time=1.67 min; ESI-MS(+) m/z [M+2H]²⁺: 1067.1.

### Preparation of Compound 2659

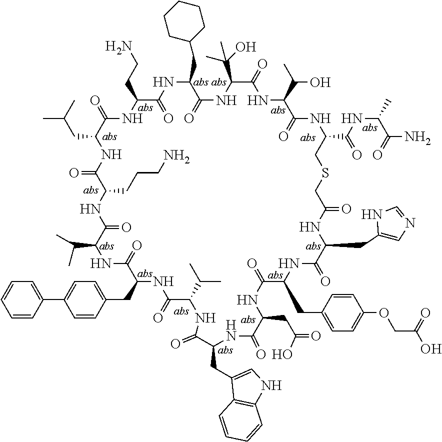 [SMILES: CC(C)C[C@H]1NC(=O)[C@H](CCCN)NC(=O)[C@H](C(C)C)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cnc[nH]2)NC(=O)CSC[C@@H](C(=O)N[C@H](C)C(N)=O)NC(=O)[C@H](C(C)O)NC(=O)[C@H](C(C)(C)O)NC(=O)[C@H](CC2CCCCC2)NC(=O)[C@H](CCN)NC1=O]

Compound 2659 was prepared on a 50 μmol scale. The yield of the product was 41.9 mg, and its estimated purity by LCMS analysis was 94.1%. Analysis condition A: Retention time=1.73 min; ESI-MS(+) m/z [M+2H]²⁺: 1005.

### Preparation of Compound 2660

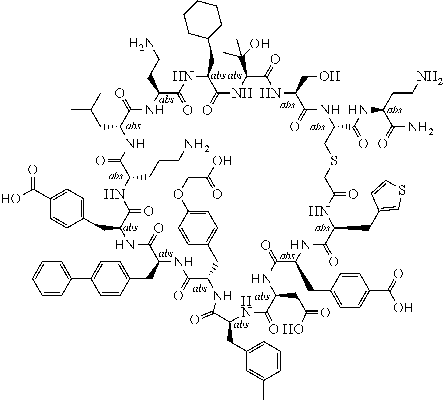 [SMILES: Cc1cccc(C[C@@H]2NC(=O)[C@H](CC(=O)O)NC(=O)[C@H](Cc3ccc(C(=O)O)cc3)NC(=O)[C@H](Cc3ccsc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Compound 2660 was prepared on a 50 μmol scale. The yield of the product was 32.8 mg, and its estimated purity by LCMS analysis was 88.9%. Analysis condition A: Retention time=1.48 min; ESI-MS(+) m/z [M+2H]²⁺: 1100.3.

### Preparation of Compound 2661

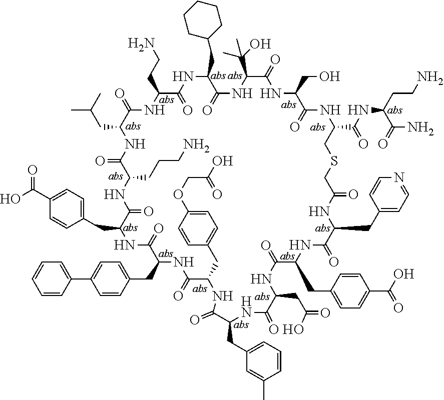 [SMILES: Cc1cccc(C[C@@H]2NC(=O)[C@H](CC(=O)O)NC(=O)[C@H](Cc3ccc(C(=O)O)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(OCC(=O)O)cc3)NC2=O)c1]

Compound 2661 was prepared on a 50 μmol scale. The yield of the product was 8.8 mg, and its estimated purity by LCMS analysis was 97.7%. Analysis condition B: Retention time=1.56 min; ESI-MS(+) m/z [M+3H]³⁺: 732.1.

### Preparation of Compound 2662

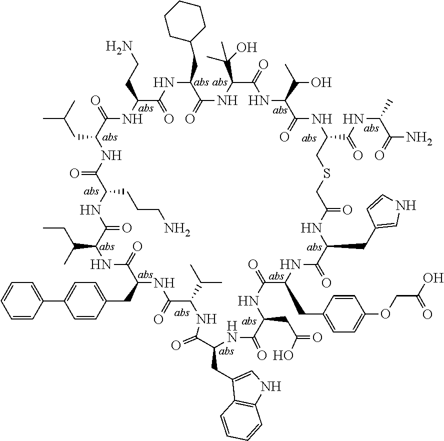 [SMILES: CCC(C)[C@@H]1NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2cc[nH]c2)NC(=O)CSC[C@@H](C(=O)N[C@H](C)C(N)=O)NC(=O)[C@H](C(C)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC1=O]

Compound 2662 was prepared on a 50 μmol scale. The yield of the product was 36.6 mg, and its estimated purity by LCMS analysis was 96.2%. Analysis condition B: Retention time=1.71 min; ESI-MS(+) m/z [M+3H]³⁺: 675.2.

### Preparation of Compound 2663

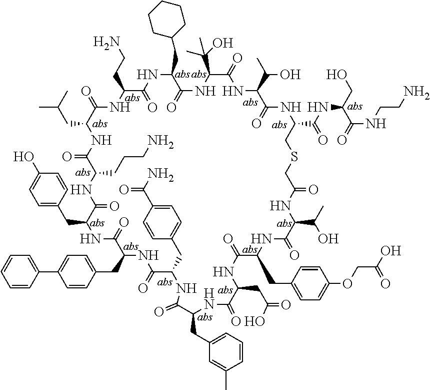 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CO)C(=O)NCCN)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2663 was prepared on a 50 μmol scale. The yield of the product was 60.4 mg, and its estimated purity by LCMS analysis was 100%. Analysis condition B: Retention time=1.62 min; ESI-MS(+) m/z [M+2H]²⁺: 1082.1.

### Preparation of Compound 2664

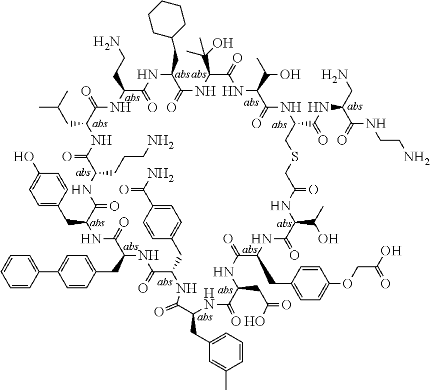 [SMILES: Cc1cccc(C[C@@H]2NC(=O)[C@H](CC(=O)O)NC(=O)[C@H](Cc3ccc(OCC(=O)O)cc3)NC(=O)[C@H](C(C)O)NC(=O)CSC[C@@H](C(=O)N[C@@H](CN)C(=O)NCCN)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(O)cc3)NC(=O)[C@H](Cc3ccc(-c4ccccc4)cc3)NC(=O)[C@H](Cc3ccc(C(N)=O)cc3)NC2=O)c1]

Compound 2664 was prepared on a 50 μmol scale. The yield of the product was 64.9 mg, and its estimated purity by LCMS analysis was 89.7%. Analysis condition A: Retention time=1.56 min; ESI-MS(+) m/z [M+2H]²⁺: 1081.3.

### Preparation of Compound 2665

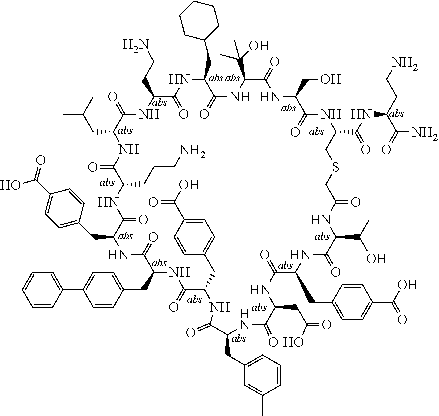 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2665 was prepared on a 50 μmol scale. The yield of the product was 12.3 mg, and its estimated purity by LCMS analysis was 98.2%. Analysis condition A: Retention time=1.42 min; ESI-MS(+) m/z [M+2H]²⁺: 1059.3.

### Preparation of Compound 2666

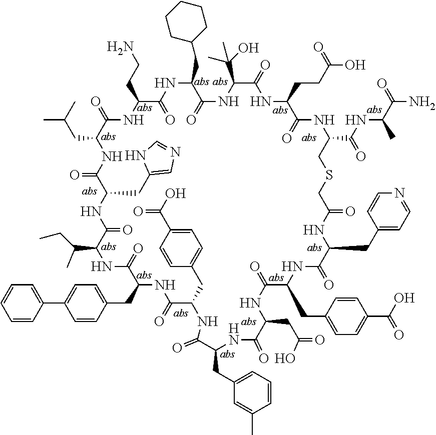 [SMILES: CCC(C)[C@@H]1NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(C(=O)O)cc2)NC(=O)[C@H](Cc2ccncc2)NC(=O)CSC[C@@H](C(=O)N[C@H](C)C(N)=O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](Cc2cnc[nH]2)NC1=O]

Compound 2666 was prepared on a 50 μmol scale. The yield of the product was 21.8 mg, and its estimated purity by LCMS analysis was 100%. Analysis condition A: Retention time=1.49 min; ESI-MS(+) m/z [M+3H]³⁺: 708.1.

### Preparation of Compound 2667

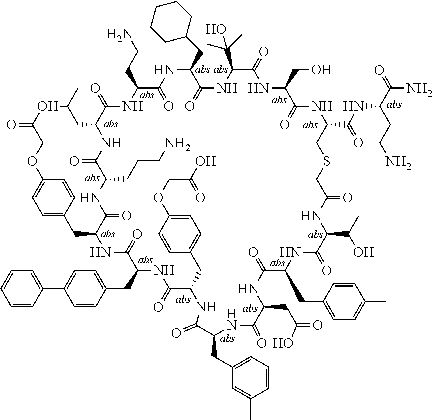 [SMILES: Cc1ccc(C[C@@H]2NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC(=O)[C@H](Cc4cccc(C)c4)NC(=O)[C@H](CC(=O)O)NC2=O)C(=O)N[C@@H](CCCN)C(=O)N3)cc1]

Compound 2667 was prepared on a 50 μmol scale. The yield of the product was 9 mg, and its estimated purity by LCMS analysis was 96.6%. Analysis condition A: Retention time=1.66 min; ESI-MS(+) m/z [M+2H]²⁺: 1074.2.

### Preparation of Compound 2668

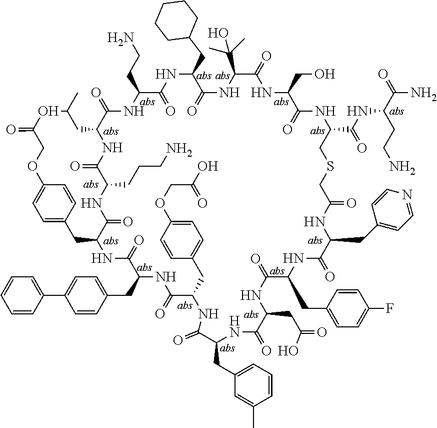 [SMILES: Cc1cccc(C[C@@H]2NC(=O)[C@H](CC(=O)O)NC(=O)[C@H](Cc3ccc(F)cc3)NC(=O)[C@H](Cc3ccncc3)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC2=O)C(=O)N[C@@H](CCCN)C(=O)N3)c1]

Compound 2668 was prepared on a 50 μmol scale. The yield of the product was 15.3 mg, and its estimated purity by LCMS analysis was 98%. Analysis condition A: Retention time=1.73 min; ESI-MS(+) m/z [M+2H]²⁺: 1100.1.

### Preparation of Compound 2669

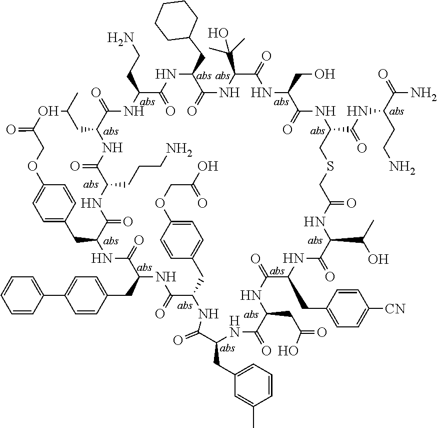 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC2=O)C(=O)N[C@@H](CCCN)C(=O)N3)c1]

Compound 2669 was prepared on a 50 μmol scale. The yield of the product was 15.9 mg, and its estimated purity by LCMS analysis was 91.8%. Analysis condition B: Retention time=1.64 min; ESI-MS(+) m/z [M+2H]²⁺: 1080.1.

### Preparation of Compound 2670

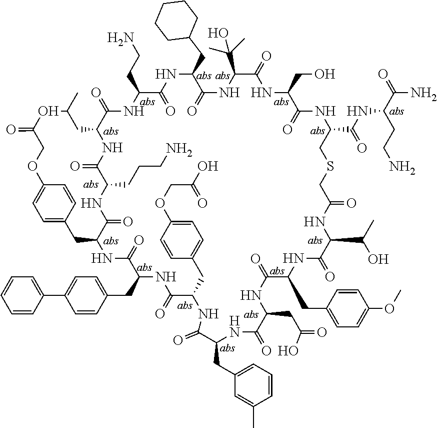 [SMILES: COc1ccc(C[C@@H]2NC(=O)[C@H](C(C)O)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC(=O)[C@H](Cc4cccc(C)c4)NC(=O)[C@H](CC(=O)O)NC2=O)C(=O)N[C@@H](CCCN)C(=O)N3)cc1]

Compound 2670 was prepared on a 50 μmol scale. The yield of the product was 16.7 mg, and its estimated purity by LCMS analysis was 97.3%. Analysis condition B: Retention time=1.74 min; ESI-MS(+) m/z [M+3H]³⁺: 722.2.

### Preparation of Compound 2671

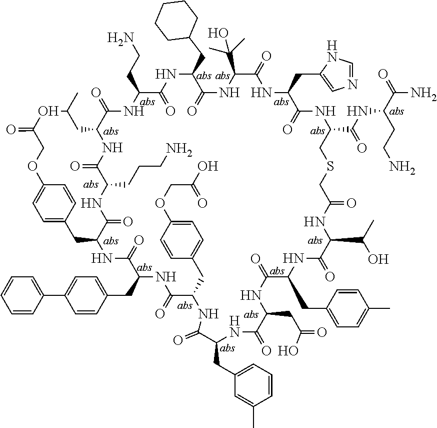 [SMILES: Cc1ccc(C[C@@H]2NC(=O)[C@H](C(C)O)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC(=O)[C@H](Cc4cccc(C)c4)NC(=O)[C@H](CC(=O)O)NC2=O)C(=O)N[C@@H](CCCN)C(=O)N3)cc1]

Compound 2671 was prepared on a 50 μmol scale. The yield of the product was 3.5 mg, and its estimated purity by LCMS analysis was 97.9%. Analysis condition B: Retention time=1.59 min; ESI-MS(+) m/z [M+2H]²⁺: 1099.3.

### Preparation of Compound 2672

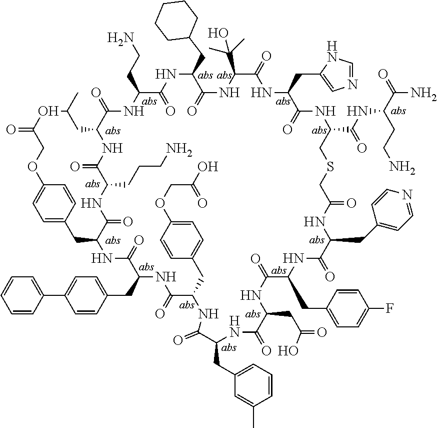 [SMILES: Cc1cccc(C[C@@H]2NC(=O)[C@H](CC(=O)O)NC(=O)[C@H](Cc3ccc(F)cc3)NC(=O)[C@H](Cc3ccncc3)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC2=O)C(=O)N[C@@H](CCCN)C(=O)N3)c1]

Compound 2672 was prepared on a 50 μmol scale. The yield of the product was 4.3 mg, and its estimated purity by LCMS analysis was 98%. Analysis condition A: Retention time=1.65 min; ESI-MS(+) m/z [M+2H]²⁺: 1125.2.

### Preparation of Compound 2673

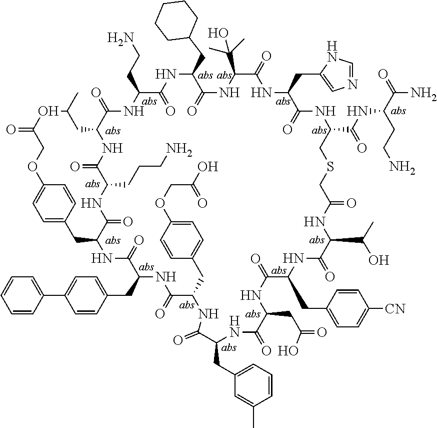 [SMILES: Cc1cccc(C[C@@H]2NC(=O)[C@H](CC(=O)O)NC(=O)[C@H](Cc3ccc(C#N)cc3)NC(=O)[C@H](C(C)O)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC2=O)C(=O)N[C@@H](CCCN)C(=O)N3)c1]

Compound 2673 was prepared on a 50 μmol scale. The yield of the product was 7.2 mg, and its estimated purity by LCMS analysis was 100%. Analysis condition A: Retention time=1.55 min; ESI-MS(+) m/z [M+2H]²⁺: 1105.

### Preparation of Compound 2674

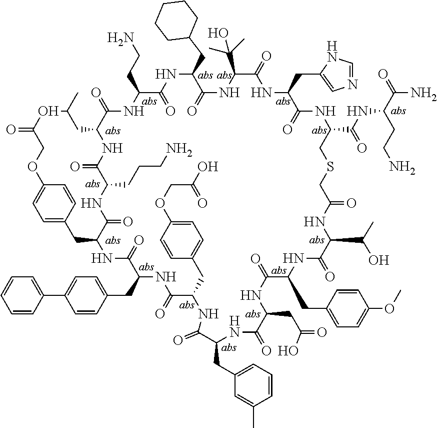 [SMILES: COc1ccc(C[C@@H]2NC(=O)[C@H](C(C)O)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC(=O)[C@H](Cc4cccc(C)c4)NC(=O)[C@H](CC(=O)O)NC2=O)C(=O)N[C@@H](CCCN)C(=O)N3)cc1]

Compound 2674 was prepared on a 50 μmol scale. The yield of the product was 3.8 mg, and its estimated purity by LCMS analysis was 96.3%. Analysis condition B: Retention time=1.55 min; ESI-MS(+) m/z [M+2H]²⁺: 1107.3.

### Preparation of Compound 2675

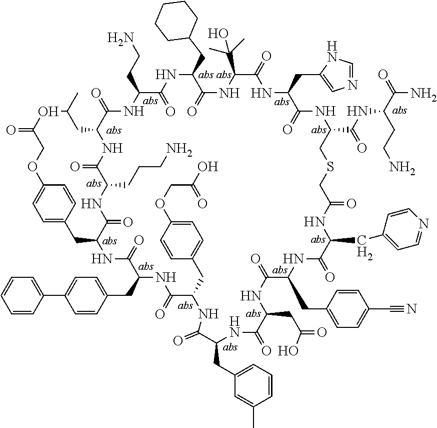 [SMILES: Cc1cccc(C[C@@H]2NC(=O)[C@H](CC(=O)O)NC(=O)[C@H](Cc3ccc(C#N)cc3)NC(=O)[C@H](Cc3ccncc3)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@H]3CC(C)OC(=O)COc4ccc(cc4)C[C@H](NC(=O)[C@H](Cc4ccc(-c5ccccc5)cc4)NC(=O)[C@H](Cc4ccc(OCC(=O)O)cc4)NC2=O)C(=O)N[C@@H](CCCN)C(=O)N3)c1]

Compound 2675 was prepared on a 50 μmol scale. The yield of the product was 3.6 mg, and its estimated purity by LCMS analysis was 95.2%. Analysis condition B: Retention time=1.52 min; ESI-MS(+) m/z [M+2H]²⁺: 1128.1.

### Preparation of Compound 2676

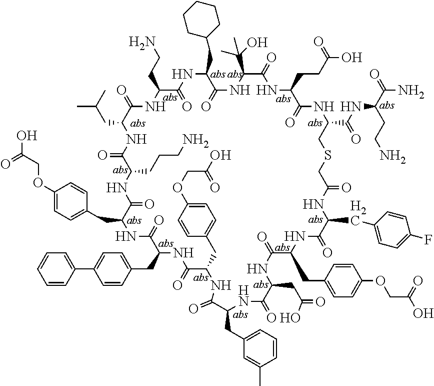 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(F)cc3)NC(=O)CSC[C@@H](C(=O)N[C@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2676 was prepared on a 50 μmol scale. The yield of the product was 45.4 mg, and its estimated purity by LCMS analysis was 95.7%. Analysis condition B: Retention time=1.62 min; ESI-MS(+) m/z [M+2H]²⁺: 1157.5.

### Preparation of Compound 2677

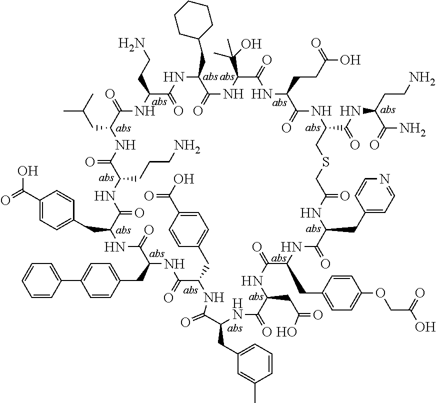 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccncc3)NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2677 was prepared on a 50 μmol scale. The yield of the product was 13.6 mg, and its estimated purity by LCMS analysis was 96.2%. Analysis condition B: Retention time=1.47 min; ESI-MS(+) m/z [M+2H]²⁺: 1119.1.

### Preparation of Compound 2678

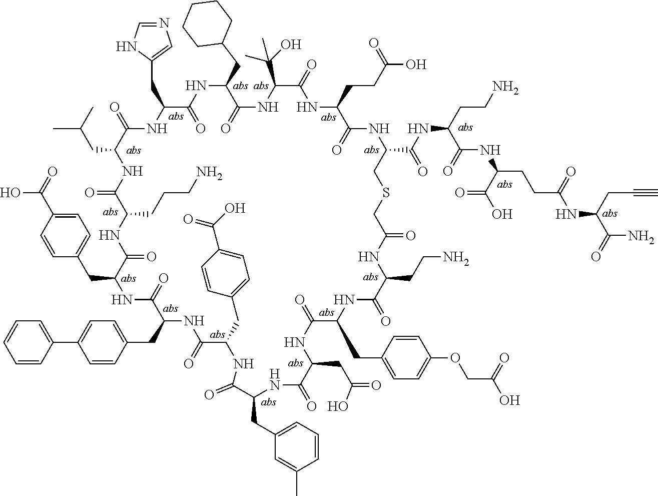 [SMILES: C#CC[C@H](NC(=O)CC[C@H](NC(=O)[C@H](CCN)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](Cc2cnc[nH]2)C(=O)N[C@@H](CC2CCCCC2)C(=O)N[C@@H](C(C)(C)O)C(=O)N[C@@H](CCC(=O)O)C(=O)N1)C(=O)O)C(N)=O]

Compound 2678 was prepared on a 50 μmol scale. The yield of the product was 78.8 mg, and its estimated purity by LCMS analysis was 92%. Analysis condition A: Retention time=1.29 min; ESI-MS(+) m/z [M+3H]³⁺: 817.1.

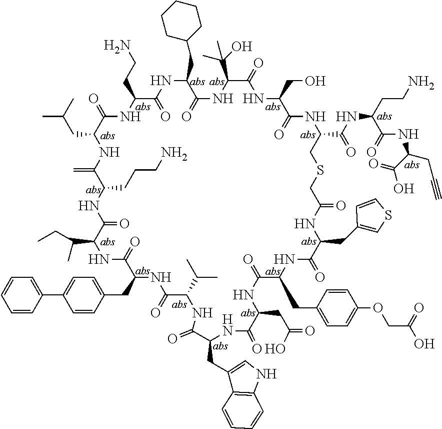 [SMILES: C#CC[C@H](NC(=O)[C@H](CCN)NC(=O)[C@@H]1CSCC(=O)N[C@@H](Cc2ccsc2)C(=O)N[C@@H](Cc2ccc(OCC(=O)O)cc2)C(=O)N[C@@H](CC(=O)O)C(=O)N[C@@H](Cc2c[nH]c3ccccc23)C(=O)N[C@@H](C(C)C)C(=O)N[C@@H](Cc2ccc(-c3ccccc3)cc2)C(=O)N[C@@H](C(C)CC)C(=O)N[C@@H](CCCN)C(=C)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2679 was prepared on a 50 μmol scale. The yield of the product was 18.6 mg, and its estimated purity by LCMS analysis was 87.2%. Analysis condition A: Retention time=1.74, 1.77 min; ESI-MS(+) m/z [M+2H]²⁺: 1076.1.

### Preparation of Compound 2680

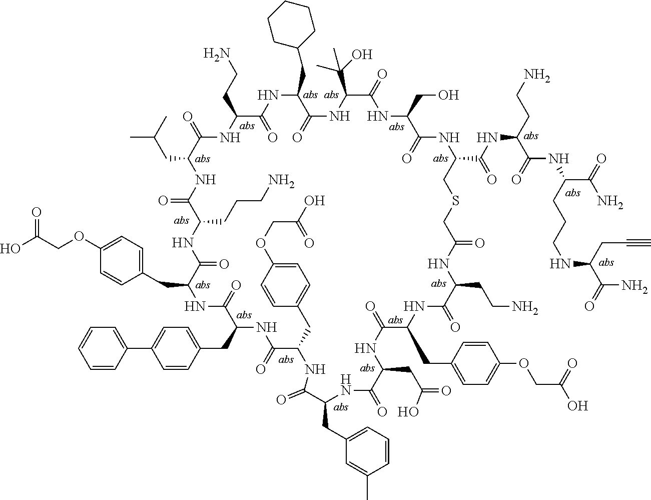 [SMILES: C#CC[C@H](NCCC[C@H](NC(=O)[C@H](CCN)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O)C(N)=O]

Compound 2680 was prepared on a 50 μmol scale. The yield of the product was 3.6 mg, and its estimated purity by LCMS analysis was 97%. Analysis condition A: Retention time=1.46 min; ESI-MS(+) m/z [M+2H]²⁺: 1216.3.

### Preparation of Compound 2681

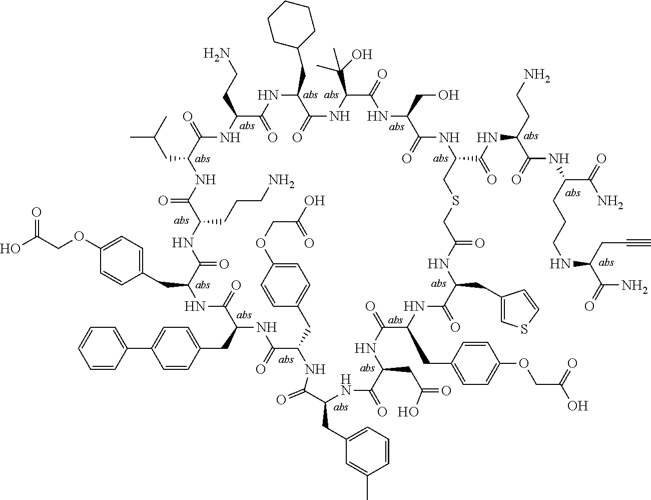 [SMILES: C#CC[C@H](NCCC[C@H](NC(=O)[C@H](CCN)NC(=O)[C@@H]1CSCC(=O)N[C@@H](Cc2ccsc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O)C(N)=O]

Compound 2681 was prepared on a 50 μmol scale. The yield of the product was 70.4 mg, and its estimated purity by LCMS analysis was 88.4%. Analysis condition A: Retention time=1.47 min; ESI-MS(+) m/z [M+2H]²⁺: 1242.1.

### Preparation of Compound 2682

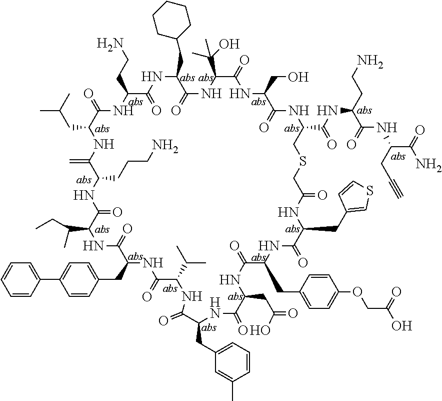 [SMILES: C#CC[C@H](NC(=O)[C@H](CCN)NC(=O)[C@@H]1CSCC(=O)N[C@@H](Cc2ccsc2)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](C(C)C)C(=O)N[C@@H](Cc2ccc(-c3ccccc3)cc2)C(=O)N[C@@H](C(C)CC)C(=O)N[C@@H](CCCN)C(=C)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(N)=O]

Compound 2682 was prepared on a 50 μmol scale. The yield of the product was 24.2 mg, and its estimated purity by LCMS analysis was 87%. Analysis condition A: Retention time=1.84 min; ESI-MS(+) m/z [M+2H]²⁺: 1063.

### Preparation of Compound 2683

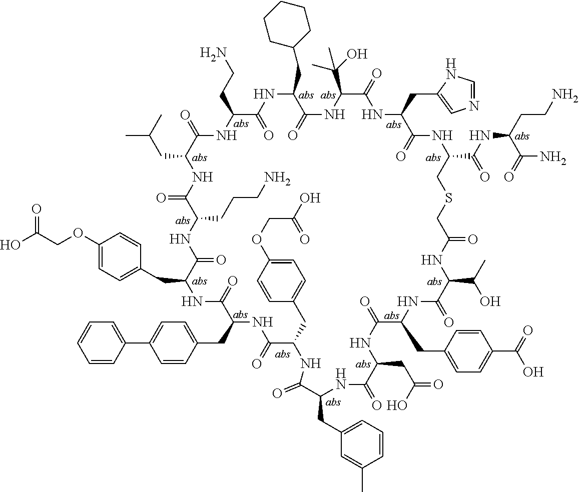 [SMILES: Cc1cccc(C[C@@H]2NC(=O)[C@H](CC(=O)O)NC(=O)[C@H](Cc3ccc(C(=O)O)cc3)NC(=O)[C@H](C(C)O)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2683 was prepared on a 50 μmol scale. The yield of the product was 9.7 mg, and its estimated purity by LCMS analysis was 96.7%. Analysis condition B: Retention time=1.6 min; ESI-MS(+) m/z [M+3H]³⁺: 743.1.

### Preparation of Compound 2684

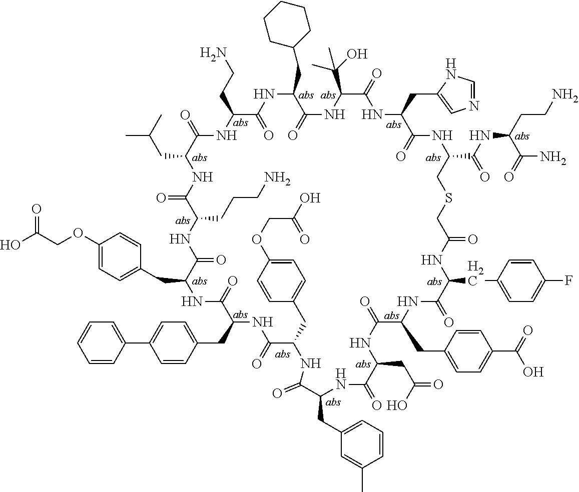 [SMILES: Cc1cccc(C[C@@H]2NC(=O)[C@H](CC(=O)O)NC(=O)[C@H](Cc3ccc(C(=O)O)cc3)NC(=O)[C@H](Cc3ccc(F)cc3)NC(=O)CSC[C@@H](C(=O)N[C@@H](CCN)C(N)=O)NC(=O)[C@H](Cc3cnc[nH]3)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2684 was prepared on a 50 μmol scale. The yield of the product was 20.4 mg, and its estimated purity by LCMS analysis was 95.3%. Analysis condition B: Retention time=1.59 min; ESI-MS(+) m/z [M+3H]³⁺: 764.6.

### Preparation of Compound 2685

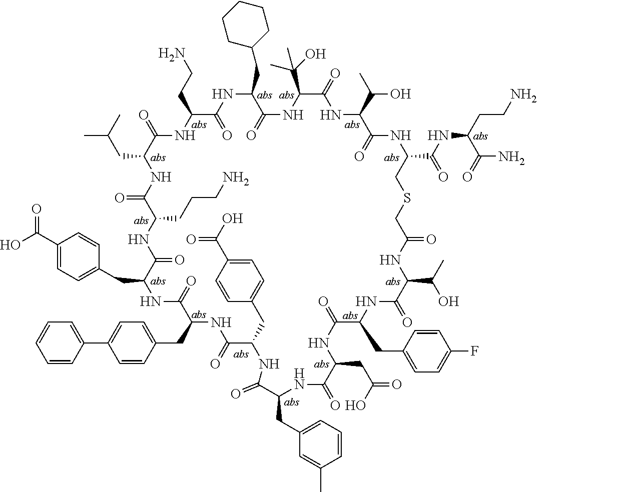 [SMILES: Cc1cccc(C[C@@H]2NC(=O)[C@H](CC(=O)O)NC(=O)[C@H](Cc3ccc(F)cc3)NC(=O)[C@H](C(C)O)NC(=O)CSC[C@@H](C(=O)N[C@@H](CCN)C(N)=O)NC(=O)[C@H](C(C)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Compound 2685 was prepared on a 50 μmol scale. The yield of the product was 25.6 mg, and its estimated purity by LCMS analysis was 100%. Analysis condition A: Retention time=1.77 min; ESI-MS(+) m/z [M+2H]²⁺: 1053.1.

### Preparation of Compound 2686

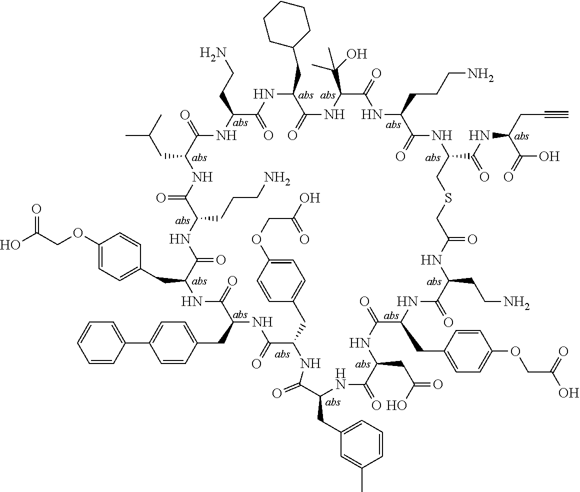 [SMILES: C#CC[C@H](NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCN)C(=O)N1)C(=O)O]

Compound 2686 was prepared on a 50 μmol scale. The yield of the product was 12.7 mg, and its estimated purity by LCMS analysis was 95.8%. Analysis condition B: Retention time=1.51 min; ESI-MS(+) m/z [M+3H]³⁺: 744.1.

### Preparation of Compound 2687

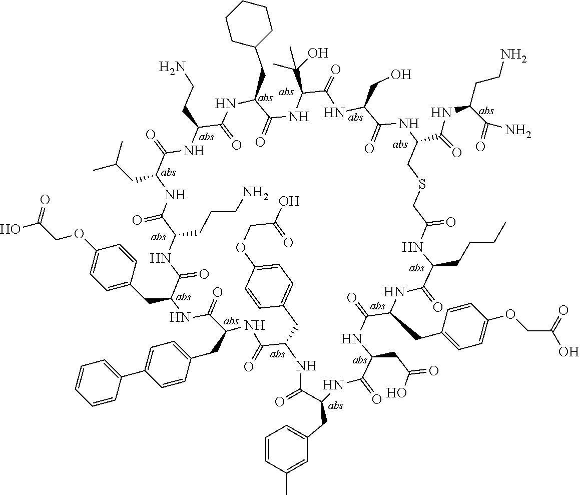 [SMILES: CCCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(OCC(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Compound 2687 was prepared on a 50 μmol scale. The yield of the product was 24.2 mg, and its estimated purity by LCMS analysis was 97.5%. Analysis condition B: Retention time=1.72 min; ESI-MS(+) m/z [M+3H]³⁺: 741.1.

### Preparation of Compound 2688

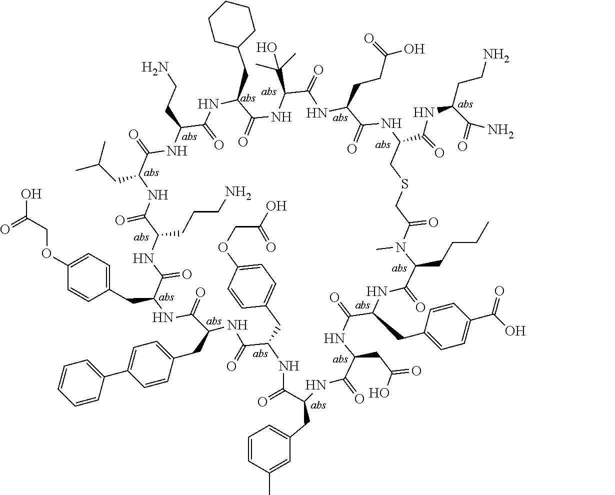 [SMILES: CCCC[C@H]1C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCC(=O)O)C(=O)N[C@H](C(=O)N[C@@H](CCN)C(N)=O)CSCC(=O)N1C]

Compound 2688 was prepared on a 50 μmol scale. The yield of the product was 31.7 mg, and its estimated purity by LCMS analysis was 98.7%. Analysis condition B: Retention time=1.7 min; ESI-MS(+) m/z [M+2H]²⁺: 1123.3.

### Preparation of Compound 2689

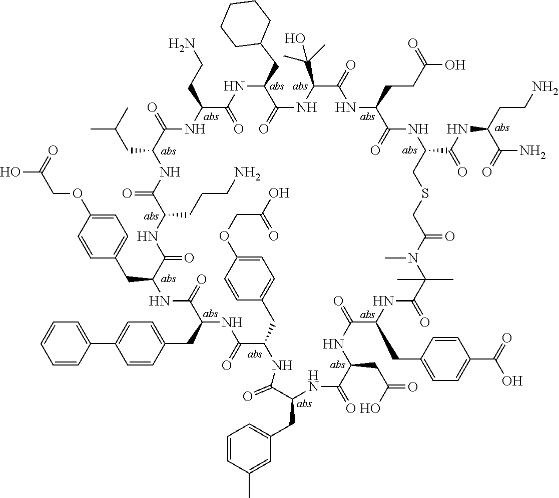 [SMILES: Cc1cccc(C[C@@H]2NC(=O)[C@H](CC(=O)O)NC(=O)[C@H](Cc3ccc(C(=O)O)cc3)NC(=O)C(C)(C)N(C)C(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2689 was prepared on a 50 μmol scale. The yield of the product was 16.7 mg, and its estimated purity by LCMS analysis was 97.5%. Analysis condition B: Retention time=1.62 min; ESI-MS(+) m/z [M+3H]³⁺: 740.1.

### Preparation of Compound 2690

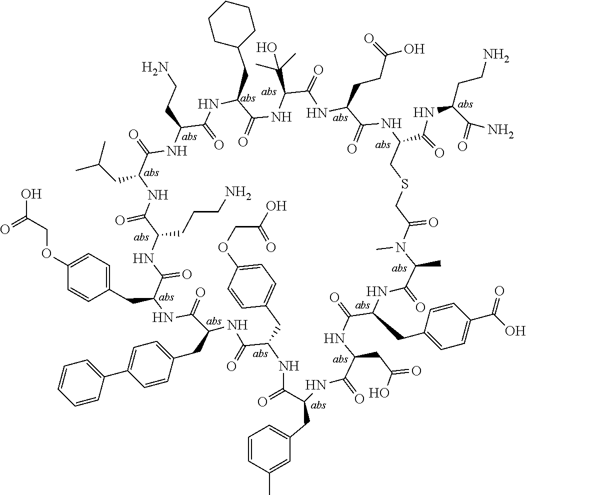 [SMILES: Cc1cccc(C[C@@H]2NC(=O)[C@H](CC(=O)O)NC(=O)[C@H](Cc3ccc(C(=O)O)cc3)NC(=O)[C@H](C)N(C)C(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2690 was prepared on a 50 μmol scale. The yield of the product was 22.2 mg, and its estimated purity by LCMS analysis was 100%. Analysis condition B: Retention time=1.65 min; ESI-MS(+) m/z [M+2H]²⁺: 1102.3.

### Preparation of Compound 2691

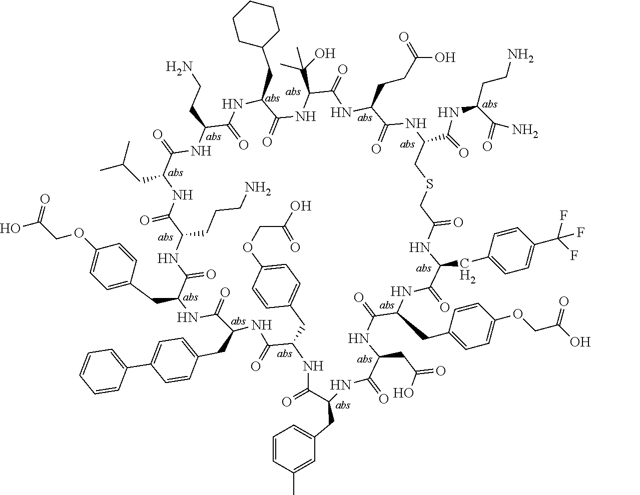 [SMILES: Cc1cccc(C[C@@H]2NC(=O)[C@H](CC(=O)O)NC(=O)[C@H](Cc3ccc(OCC(=O)O)cc3)NC(=O)[C@H](Cc3ccc(C(F)(F)F)cc3)NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2691 was prepared on a 50 μmol scale. The yield of the product was 14.7 mg, and its estimated purity by LCMS analysis was 97.2%. Analysis condition A: Retention time=1.52 min; ESI-MS(+) m/z [M+3H]³⁺: 788.4.

### Preparation of Compound 2692

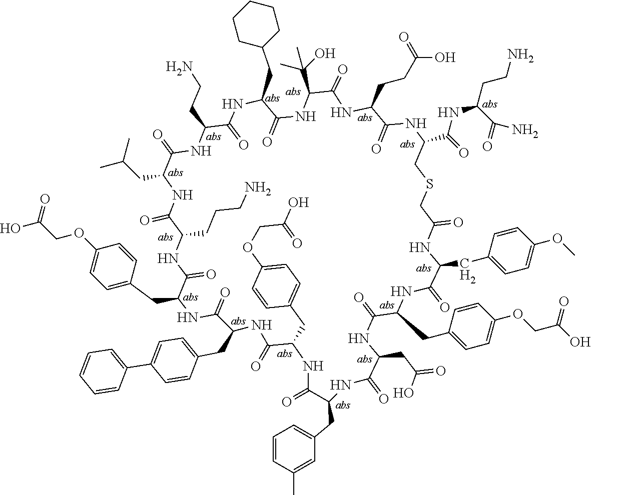 [SMILES: COc1ccc(C[C@@H]2NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC(=O)[C@H](Cc3cccc(C)c3)NC(=O)[C@H](CC(=O)O)NC(=O)[C@H](Cc3ccc(OCC(=O)O)cc3)NC2=O)cc1]

Compound 2692 was prepared on a 50 μmol scale. The yield of the product was 28.2 mg, and its estimated purity by LCMS analysis was 89.1%. Analysis condition A: Retention time=1.42 min; ESI-MS(+) m/z [M+2H]²⁺: 1163.2.

### Preparation of Compound 2693

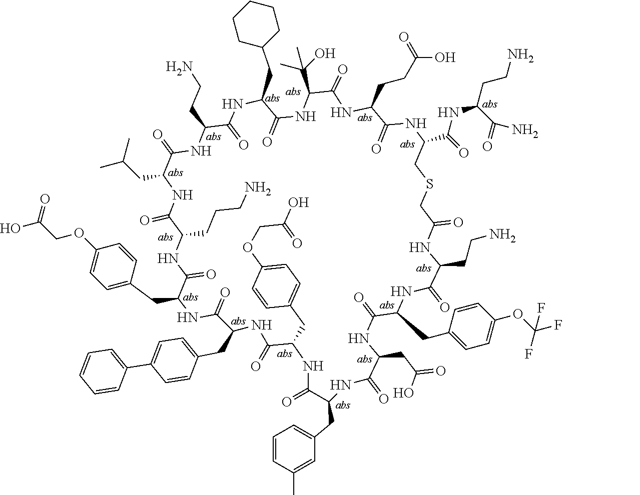 [SMILES: Cc1cccc(C[C@@H]2NC(=O)[C@H](CC(=O)O)NC(=O)[C@H](Cc3ccc(OC(F)(F)F)cc3)NC(=O)[C@H](CCN)NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OCC(=O)O)cc3)NC(=O)[C@H](Cc3ccc(-c4ccccc4)cc3)NC(=O)[C@H](Cc3ccc(OCC(=O)O)cc3)NC2=O)c1]

Compound 2693 was prepared on a 50 μmol scale. The yield of the product was 16.8 mg, and its estimated purity by LCMS analysis was 96.6%. Analysis condition B: Retention time=1.76 min; ESI-MS(+) m/z [M+2H]²⁺: 1130.3.

### Preparation of Compound 2694

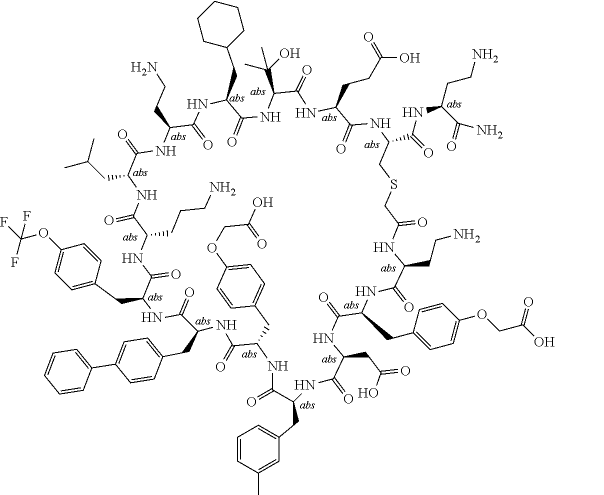 [SMILES: Cc1cccc(C[C@@H]2NC(=O)[C@H](CC(=O)O)NC(=O)[C@H](Cc3ccc(OCC(=O)O)cc3)NC(=O)[C@H](CCN)NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(OC(F)(F)F)cc3)NC(=O)[C@H](Cc3ccc(-c4ccccc4)cc3)NC(=O)[C@H](Cc3ccc(OCC(=O)O)cc3)NC2=O)c1]

Compound 2694 was prepared on a 50 μmol scale. The yield of the product was 14.4 mg, and its estimated purity by LCMS analysis was 97.6%. Analysis condition A: Retention time=1.81 min; ESI-MS(+) m/z [M+2H]²⁺: 1130.2.

### Preparation of Compound 2695

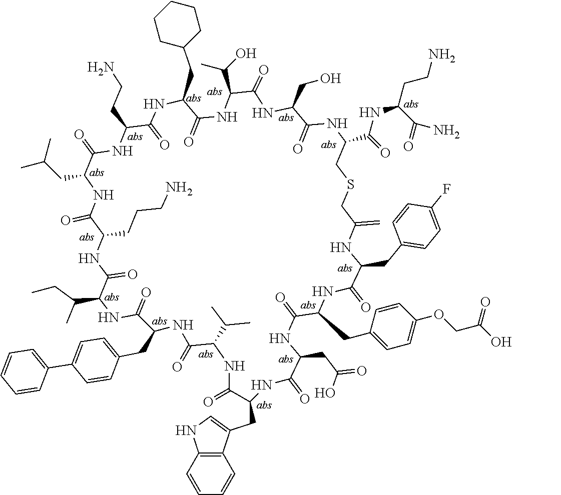 [SMILES: C=C1CSC[C@@H](C(=O)N[C@@H](CCN)C(N)=O)NC(=O)[C@H](CO)NC(=O)[C@H](C(C)O)NC(=O)[C@H](CC2CCCCC2)NC(=O)[C@H](CCN)NC(=O)[C@@H](CC(C)C)NC(=O)[C@H](CCCN)NC(=O)[C@H](C(C)CC)NC(=O)[C@H](Cc2ccc(-c3ccccc3)cc2)NC(=O)[C@H](C(C)C)NC(=O)[C@H](Cc2c[nH]c3ccccc23)NC(=O)[C@H](CC(=O)O)NC(=O)[C@H](Cc2ccc(OCC(=O)O)cc2)NC(=O)[C@H](Cc2ccc(F)cc2)N1]

Compound 2695 was prepared on a 50 μmol scale. The yield of the product was 10.3 mg, and its estimated purity by LCMS analysis was 91%. Analysis condition A: Retention time=1.81 min; ESI-MS(+) m/z [M+2H]²⁺: 1026.1.

### Preparation of Compound 2696

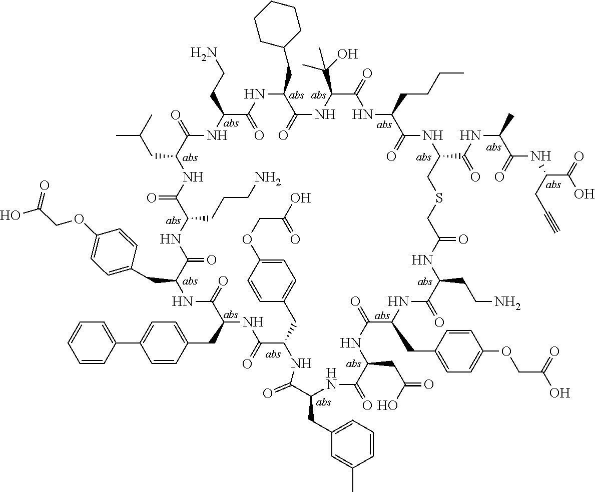 [SMILES: C#CC[C@H](NC(=O)[C@H](C)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCCC)C(=O)N1)C(=O)O]

Compound 2696 was prepared on a 50 μmol scale. The yield of the product was 36.5 mg, and its estimated purity by LCMS analysis was 95.8%. Analysis condition A: Retention time=1.55 min; ESI-MS(+) m/z [M+3H]³⁺: 767.

### Preparation of Compound 2697

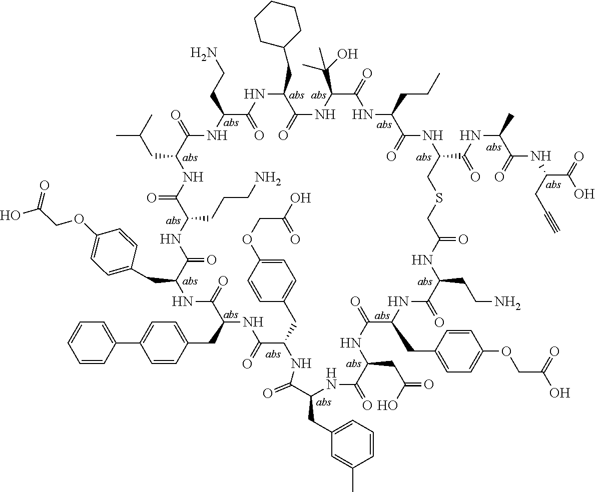 [SMILES: C#CC[C@H](NC(=O)[C@H](C)NC(=O)[C@@H]1CSCC(=O)N[C@@H](CCN)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CCC)C(=O)N1)C(=O)O]

Compound 2697 was prepared on a 50 μmol scale. The yield of the product was 12.7 mg, and its estimated purity by LCMS analysis was 97.4%. Analysis condition A: Retention time=1.42 min; ESI-MS(+) m/z [M+2H]²⁺: 1143.2.

### Preparation of Compound 2698

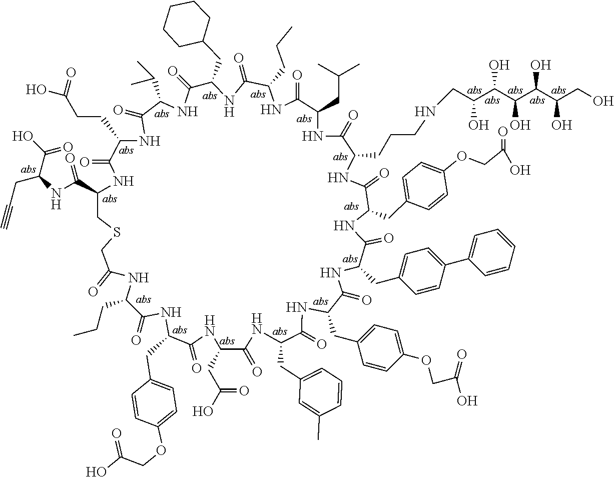 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCNC[C@@H](O)[C@H](O)[C@H](O)[C@@H](O)[C@H](O)CO)C(=O)N[C@H](CC(C)C)C(=O)N[C@@H](CCC)C(=O)N[C@@H](CC2CCCCC2)C(=O)N[C@@H](C(C)C)C(=O)N[C@@H](CCC(=O)O)C(=O)N1)C(=O)O]

Compound 2698 was prepared on a 6.7 μmol scale. The yield of the product was 7.3 mg, and its estimated purity by LCMS analysis was 91.9%. Analysis condition A: Retention time=1.34 min; ESI-MS(+) m/z [M-3H] 3:805.4.

### Preparation of Compound 2699

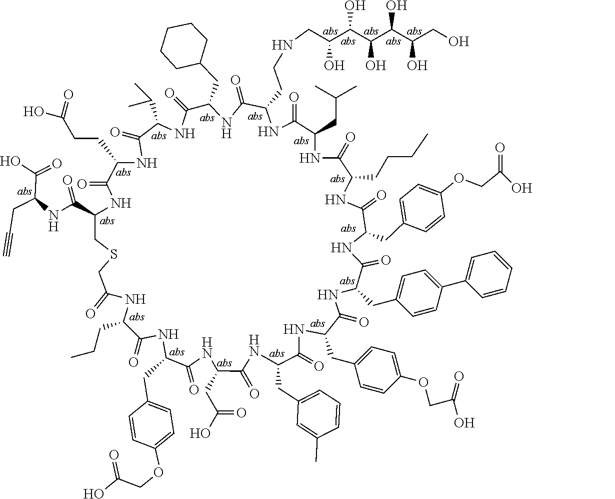 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCC)C(=O)N[C@H](CC(C)C)C(=O)N[C@@H](CCNC[C@@H](O)[C@H](O)[C@H](O)[C@@H](O)[C@H](O)CO)C(=O)N[C@@H](CC2CCCCC2)C(=O)N[C@@H](C(C)C)C(=O)N[C@@H](CCC(=O)O)C(=O)N1)C(=O)O]

Compound 2699 was prepared on a 1.8 μmol scale. The yield of the product was 1.3 mg, and its estimated purity by LCMS analysis was 93.5%. Analysis condition B: Retention time=1.36 min; ESI-MS(+) m/z [M+2H]²⁺: 1211.0.

### Preparation of Compound 2700

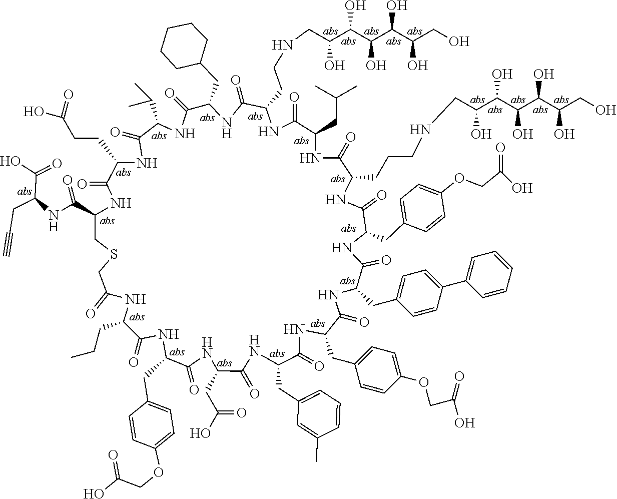 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCNC[C@@H](O)[C@H](O)[C@H](O)[C@@H](O)[C@H](O)CO)C(=O)N[C@H](CC(C)C)C(=O)N[C@@H](CCNC[C@@H](O)[C@H](O)[C@H](O)[C@@H](O)[C@H](O)CO)C(=O)N[C@@H](CC2CCCCC2)C(=O)N[C@@H](C(C)C)C(=O)N[C@@H](CCC(=O)O)C(=O)N1)C(=O)O]

Compound 2700 was prepared on a 5.8 μmol scale. The yield of the product was 4.4 mg, and its estimated purity by LCMS analysis was 80.1%. Analysis condition B: Retention time=1.66 min; ESI-MS(+) m/z [M-3H] 3:870.3.

### Preparation of Compound 2701

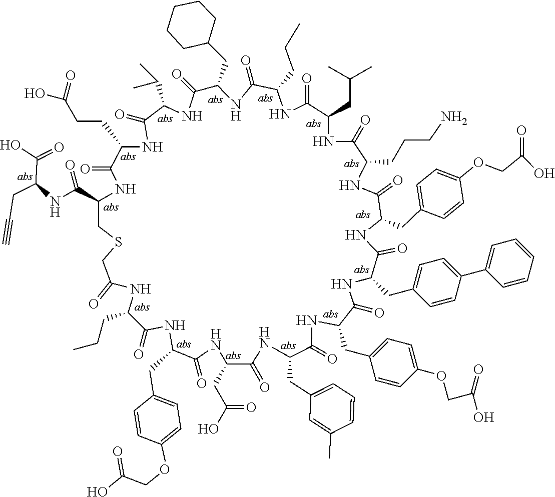 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCC)C(=O)N[C@@H](CC2CCCCC2)C(=O)N[C@@H](C(C)C)C(=O)N[C@@H](CCC(=O)O)C(=O)N1)C(=O)O]

Compound 2701 was prepared on a 100 μmol scale. The yield of the product was 22.8 mg, and its estimated purity by LCMS analysis was 95.8%. Analysis condition A: Retention time=1.36 min; ESI-MS(+) m/z [M+2H]²⁺: 1113.9.

### Preparation of Compound 2702

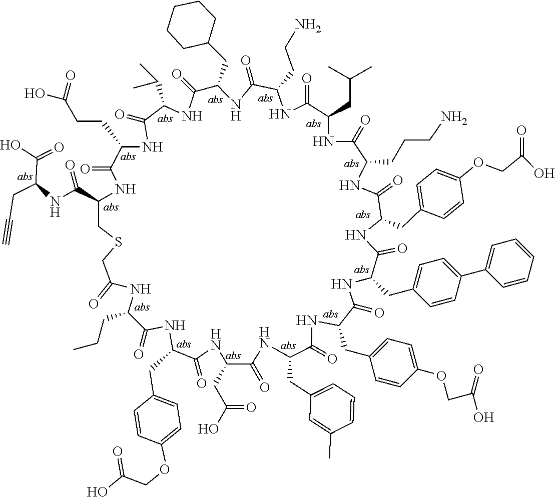 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 2702 was prepared on a 100 μmol scale. The yield of the product was 16.1 mg, and its estimated purity by LCMS analysis was 97.6%. Analysis condition B: Retention time=1.66 min; ESI-MS(+) m/z [M+2H]²⁺: 1114.3.

### Preparation of Compound 2703

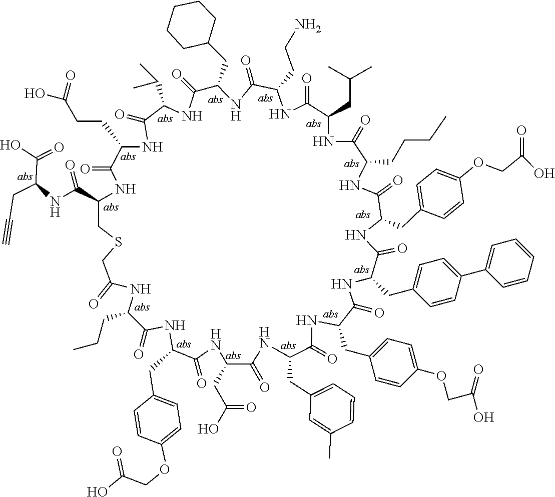 [SMILES: C#CC[C@H](NC(=O)[C@@H]1CSCC(=O)N[C@@H](CCC)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCC)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 2703 was prepared on a 100 μmol scale. The yield of the product was 4.8 mg, and its estimated purity by LCMS analysis was 93.1%. Analysis condition B: Retention time=1.93 min; ESI-MS(+) m/z [M+2H]²⁺: 1114.

### Preparation of Compound 2704

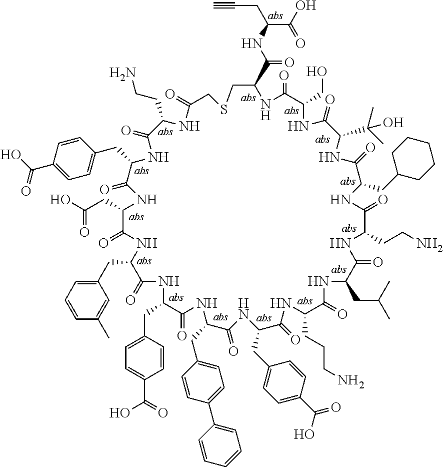 [SMILES: C#CC[C@H](NC(=O)[C@@H]1CSCC(=O)N[C@@H](CCN)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2704 was prepared on a 100 μmol scale. The yield of the product was 5.3 mg, and its estimated purity by LCMS analysis was 87.4%. Analysis condition B: Retention time=1.52 min; ESI-MS(+) m/z [M+2H]²⁺: 1056.6.

### Preparation of Compound 2705

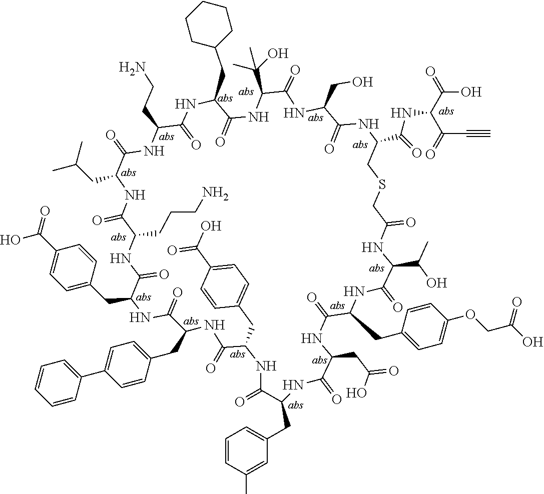 [SMILES: C#CC(=O)[C@H](NC(=O)[C@@H]1CSCC(=O)N[C@@H](C(C)O)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)(C)O)C(=O)N[C@@H](CO)C(=O)N1)C(=O)O]

Compound 2705 was prepared on a 100 μmol scale. The yield of the product was 35.2 mg, and its estimated purity by LCMS analysis was 92.1%. Analysis condition B: Retention time=1.58 min; ESI-MS(+) m/z [M+2H]²⁺: 1072.1.

### Preparation of Compound 2706

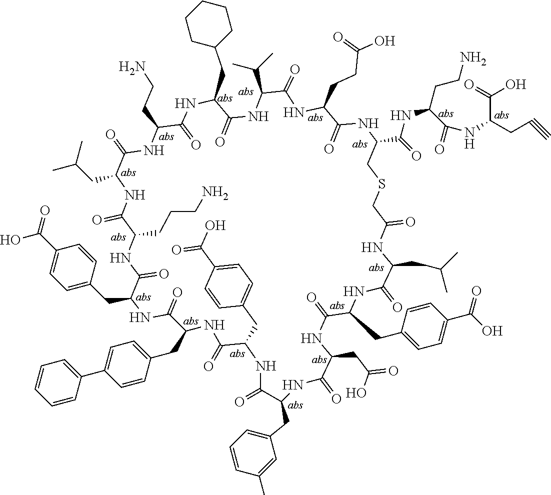 [SMILES: C#CC[C@H](NC(=O)[C@H](CCN)NC(=O)[C@@H]1CSCC(=O)N[C@@H](CC(C)C)C(=O)N[C@@H](Cc2ccc(C(=O)O)cc2)C(=O)N[C@@H](CC(=O)O)C(=O)N[C@@H](Cc2cccc(C)c2)C(=O)N[C@@H](Cc2ccc(C(=O)O)cc2)C(=O)N[C@@H](Cc2ccc(-c3ccccc3)cc2)C(=O)N[C@@H](Cc2ccc(C(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 2706 was prepared on a 100 μmol scale. The yield of the product was 41 mg, and its estimated purity by LCMS analysis was 89.7%. Analysis condition B: Retention time=1.58 min; ESI-MS(+) m/z [M+3H]³⁺: 751.3.

### Preparation of Compound 2707

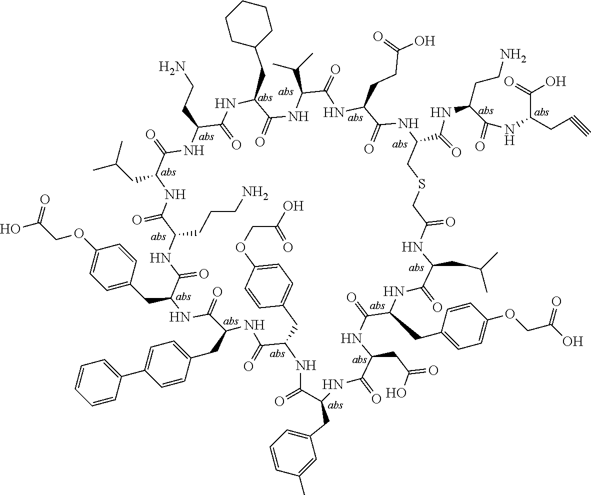 [SMILES: C#CC[C@H](NC(=O)[C@H](CCN)NC(=O)[C@@H]1CSCC(=O)N[C@@H](CC(C)C)C(=O)N[C@@H](Cc2ccc(OCC(=O)O)cc2)C(=O)N[C@@H](CC(=O)O)C(=O)N[C@@H](Cc2cccc(C)c2)C(=O)N[C@@H](Cc2ccc(OCC(=O)O)cc2)C(=O)N[C@@H](Cc2ccc(-c3ccccc3)cc2)C(=O)N[C@@H](Cc2ccc(OCC(=O)O)cc2)C(=O)N[C@@H](CCCN)C(=O)N[C@H](CC(C)C)C(=O)N[C@@H](CCN)C(=O)N[C@@H](CC2CCCCC2)C(=O)N[C@@H](C(C)C)C(=O)N[C@@H](CCC(=O)O)C(=O)N1)C(=O)O]

Compound 2707 was prepared on a 100 μmol scale. The yield of the product was 59.8 mg, and its estimated purity by LCMS analysis was 84.1%. Analysis condition B: Retention time=1.58 min; ESI-MS(+) m/z [M+2H]²⁺: 1171.3.

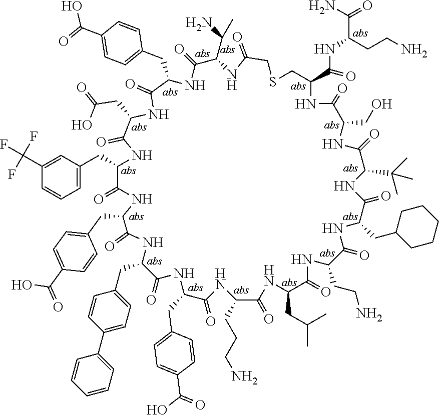 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H]([C@H](C)N)NC(=O)CSC[C@@H](C(=O)N[C@@H](CCN)C(N)=O)NC(=O)[C@H](CO)NC(=O)[C@H](C(C)(C)C)NC(=O)[C@H](CC2CCCCC2)NC(=O)[C@H](CCN)NC1=O]

Example 2708 was prepared on a 200 μmol scale. The yield of the product was 46.2 mg, and its estimated purity by LCMS analysis was 97%. Analysis condition B: Retention time=1.56 min; ESI-MS(+) m/z [M+2H]²⁺: 1085.8.

### Preparation of Example 2709

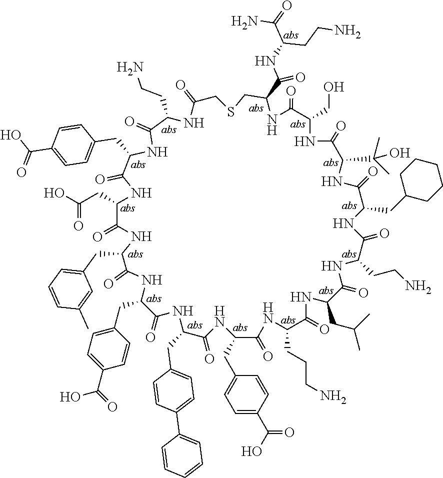 [SMILES: Cc1cccc(C[C@@H]2NC(=O)[C@H](CC(=O)O)NC(=O)[C@H](Cc3ccc(C(=O)O)cc3)NC(=O)[C@H](CCN)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2709 was prepared on a 200 μmol scale. The yield of the product was 7 mg, and its estimated purity by LCMS analysis was 100%. Analysis condition A: Retention time=1.44 min; ESI-MS(+) m/z [M+2H]²⁺: 1058.8.

### Preparation of Example 2710

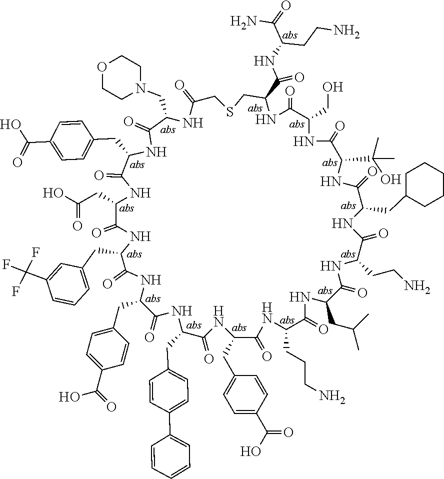 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2710 was prepared on a 200 μmol scale. The yield of the product was 34.8 mg, and its estimated purity by LCMS analysis was 93.3%. Analysis condition A: Retention time=1.47 min; ESI-MS(+) m/z [M+2H]²⁺: 1113.9.

### Preparation of Example 2711

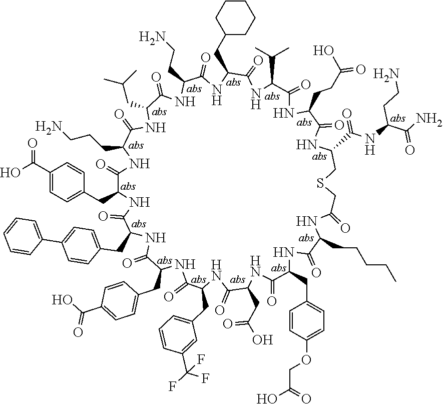 [SMILES: CCCCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 2711 was prepared on a 50 μmol scale. The yield of the product was 8.1 mg, and its estimated purity by LCMS analysis was 95.9%. Analysis condition B: Retention time=1.75 min; ESI-MS(+) m/z [M+2H]²⁺: 1120.4.

### Preparation of Example 2712

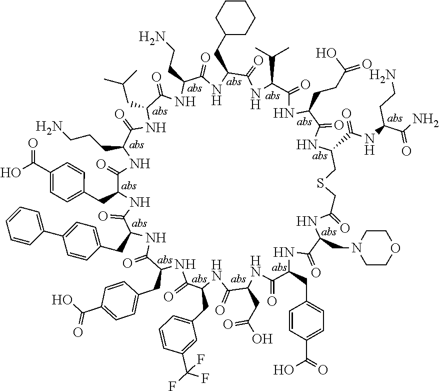 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2712 was prepared on a 50 μmol scale. The yield of the product was 6.4 mg, and its estimated purity by LCMS analysis was 95.1%. Analysis condition A: Retention time=1.46 min; ESI-MS(+) m/z [M+3H]³⁺: 751.6.

### Preparation of Example 2713

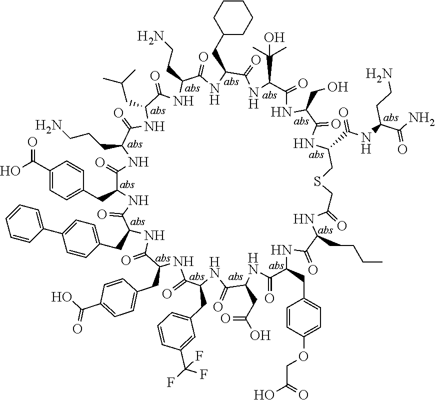 [SMILES: CCCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 2713 was prepared on a 50 μmol scale. The yield of the product was 11.8 mg, and its estimated purity by LCMS analysis was 97.2%. Analysis condition B: Retention time=1.76 min; ESI-MS(+) m/z [M+2H]²⁺: 1107.1.

### Preparation of Example 2714

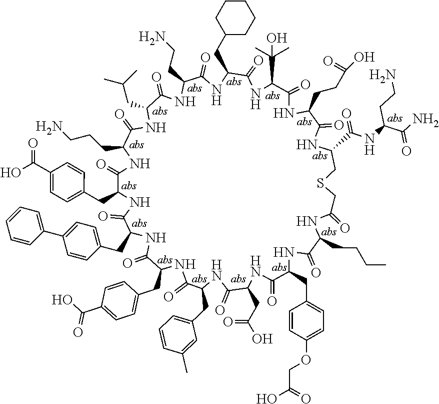 [SMILES: CCCC[C@@H]1NC(=O)CSC[C@@H](C(=O)N[C@@H](CCN)C(N)=O)NC(=O)[C@H](CCC(=O)O)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(=O)O)NC(=O)[C@H](Cc2ccc(OCC(=O)O)cc2)NC1=O]

Example 2714 was prepared on a 50 μmol scale. The yield of the product was 5.8 mg, and its estimated purity by LCMS analysis was 97.6%. Analysis condition A: Retention time=1.48 min; ESI-MS(+) m/z [M+2H]²⁺: 1093.1.

### Preparation of Example 2715

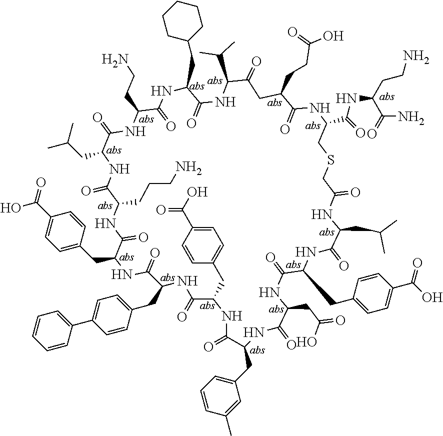 [SMILES: Cc1cccc(C[C@@H]2NC(=O)[C@H](CC(=O)O)NC(=O)[C@H](Cc3ccc(C(=O)O)cc3)NC(=O)[C@H](CC(C)C)NC(=O)CSC[C@@H](C(=O)N[C@@H](CCN)C(N)=O)NC(=O)[C@H](CCC(=O)O)CC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2715 was prepared on a 100 μmol scale. The yield of the product was 51.9 mg, and its estimated purity by LCMS analysis was 84.6%. Analysis condition A: Retention time=1.46 min; ESI-MS(+) m/z [M+2H]²⁺: 1078.2.

### Preparation of Example 2716

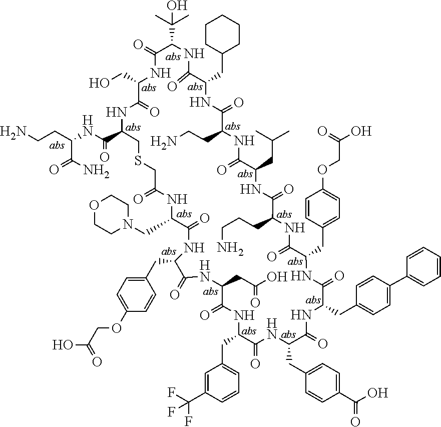 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)C[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2716 was prepared on a 50 μmol scale. The yield of the product was 17.9 mg, and its estimated purity by LCMS analysis was 100%. Analysis condition A: Retention time=1.47 min; ESI-MS(+) m/z [M+2H]²⁺: 1144.1.

### Preparation of Example 2717

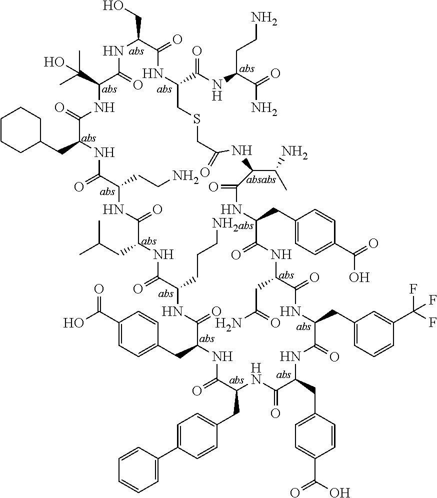 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(C(=O)O)cc2)NC(=O)[C@H]([C@@H](C)N)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2717 was prepared on a 50 μmol scale. The yield of the product was 3.2 mg, and its estimated purity by LCMS analysis was 92%. Analysis condition A: Retention time=1.59 min; ESI-MS(+) m/z [M+3H]³⁺: 723.8.

### Preparation of Example 2718

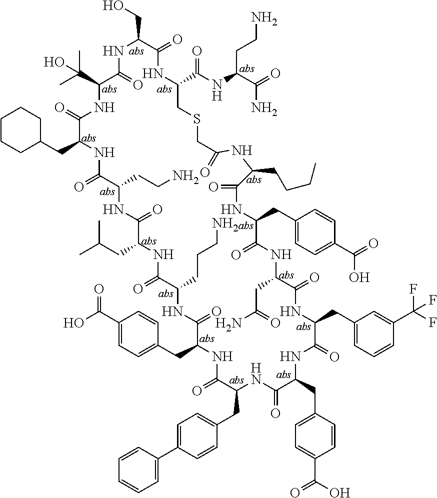 [SMILES: CCCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(C(=O)O)cc2)NC1=O]

Example 2718 was prepared on a 50 μmol scale. The yield of the product was 7.6 mg, and its estimated purity by LCMS analysis was 81.6%. Analysis condition A: Retention time=1.64 min; ESI-MS(+) m/z²⁺: 1091.6.

### Preparation of Example 2719

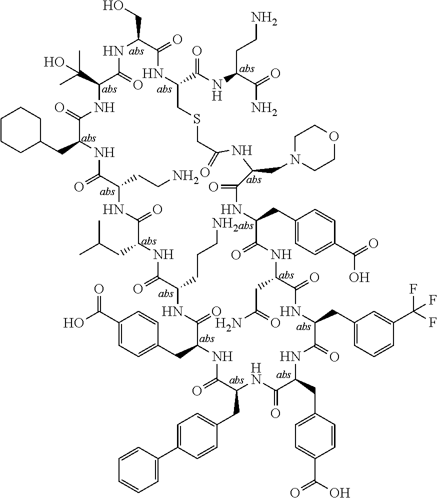 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2719 was prepared on a 50 μmol scale. The yield of the product was 6.9 mg, and its estimated purity by LCMS analysis was 92.4%. Analysis condition A: Retention time=1.59 min; ESI-MS(+) m/z [M+2H]²⁺: 1113.4.

### Preparation of Example 2720

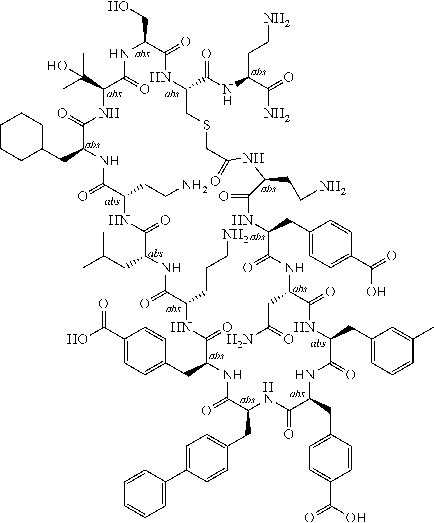 [SMILES: Cc1cccc(C[C@@H]2NC(=O)[C@H](CC(N)=O)NC(=O)[C@H](Cc3ccc(C(=O)O)cc3)NC(=O)[C@H](CCN)NC(=O)CSC[C@@H](C(=O)N[C@@H](CCN)C(N)=O)NC(=O)[C@H](CO)NC(=O)[C@H](C(C)(C)O)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2720 was prepared on a 50 μmol scale. The yield of the product was 3.6 mg, and its estimated purity by LCMS analysis was 85.4%. Analysis condition B: Retention time=1.48 min; ESI-MS(+) m/z [M+2H]²⁺: 1058.4.

### Preparation of Example 2721

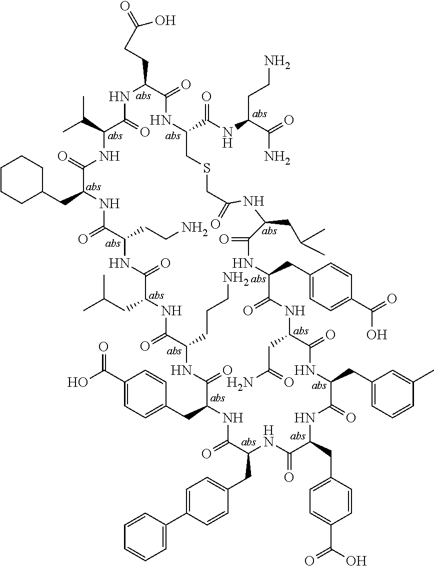 [SMILES: Cc1cccc(C[C@@H]2NC(=O)[C@H](CC(N)=O)NC(=O)[C@H](Cc3ccc(C(=O)O)cc3)NC(=O)[C@H](CC(C)C)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC(=O)[C@H](Cc3ccc(C(=O)O)cc3)NC2=O)c1]

Example 2721 was prepared on a 50 μmol scale. The yield of the product was 8.4 mg, and its estimated purity by LCMS analysis was 86.4%. Analysis condition B: Retention time=1.59 min; ESI-MS(+) m/z [M+2H]²⁺: 1078.

### Preparation of Example 2722

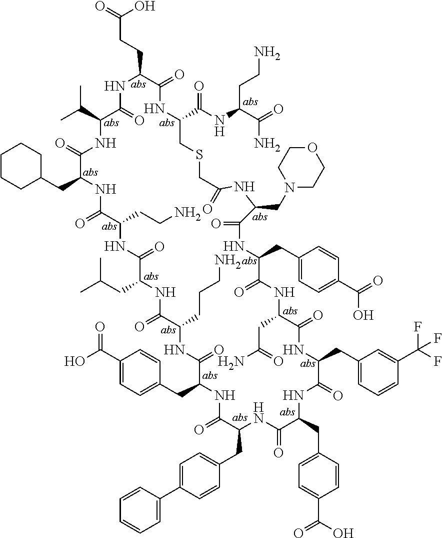 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2722 was prepared on a 50 μmol scale. The yield of the product was 19.3 mg, and its estimated purity by LCMS analysis was 100%. Analysis condition A: Retention time=1.47 min; ESI-MS(+) m/z [M+2H]²⁺: 1126.5.

### Preparation of Example 2723

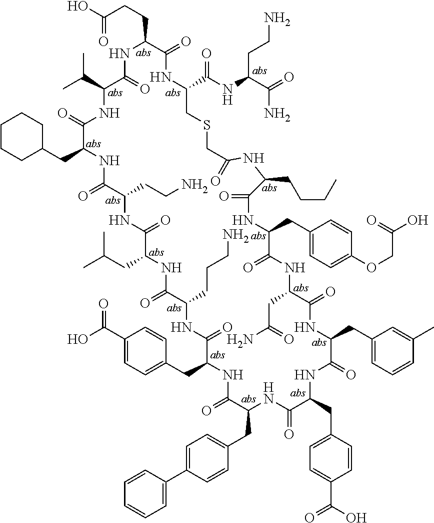 [SMILES: CCCC[C@@H]1NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C)c2)NC(=O)[C@H](CC(N)=O)NC(=O)[C@H](Cc2ccc(OCC(=O)O)cc2)NC1=O]

Example 2723 was prepared on a 50 μmol scale. The yield of the product was 12.7 mg, and its estimated purity by LCMS analysis was 88.1%. Analysis condition A: Retention time=1.53 min; ESI-MS(+) m/z [M+2H]²⁺: 1093.

### Preparation of Example 2724

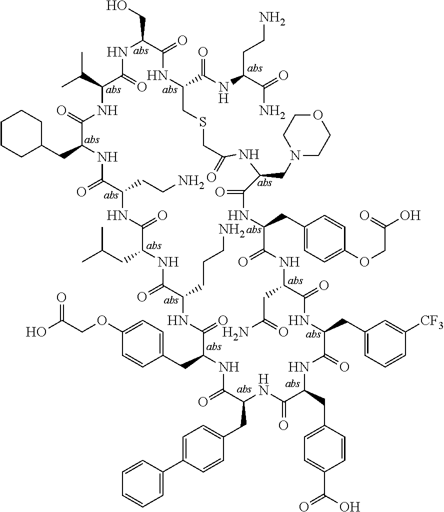 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)C)NC(=O)[C@H](CC2CCCCC2)NC(=O)[C@H](CCN)NC1=O]

Example 2724 was prepared on a 50 μmol scale. The yield of the product was 8.4 mg, and its estimated purity by LCMS analysis was 96.8%. Analysis condition A: Retention time=1.61 min; ESI-MS(+) m/z [M+2H]²⁺: 1143.3.

### Preparation of Example 2725

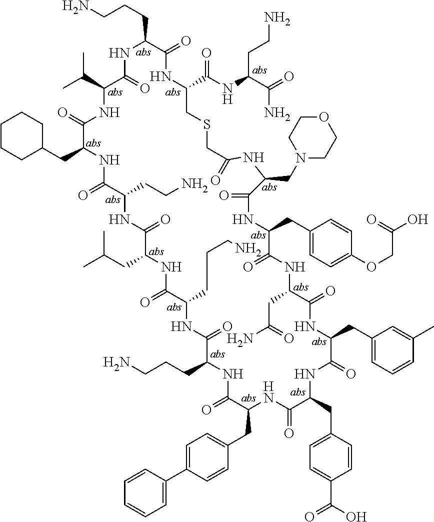 [SMILES: Cc1cccc(C[C@@H]2NC(=O)[C@H](CC(N)=O)NC(=O)[C@H](Cc3ccc(OCC(=O)O)cc3)NC(=O)[C@H](CN3CCOCC3)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](Cc3ccc(C(=O)O)cc3)NC2=O)c1]

Example 2725 was prepared on a 50 μmol scale. The yield of the product was 9.8 mg, and its estimated purity by LCMS analysis was 92.1%. Analysis condition A: Retention time=1.5 min; ESI-MS(+) m/z [M+2H]²⁺: 1068.5.

### Preparation of Example 2726

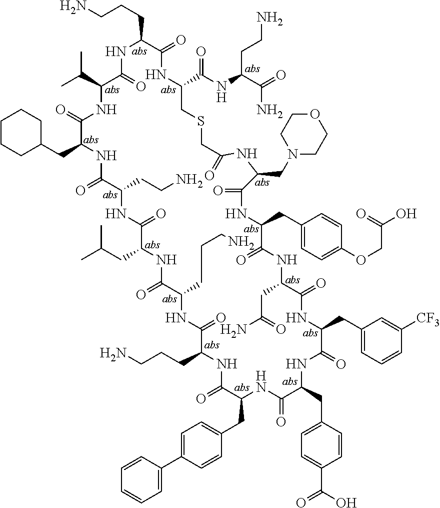 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CCCN)NC(=O)[C@H](C(C)C)NC(=O)[C@H](CC2CCCCC2)NC(=O)[C@H](CCN)NC1=O]

Example 2726 was prepared on a 50 μmol scale. The yield of the product was 11.1 mg, and its estimated purity by LCMS analysis was 91%. Analysis condition A: Retention time=1.53 min; ESI-MS(+) m/z [M+2H]²⁺: 1095.6.

### Preparation of Example 2727

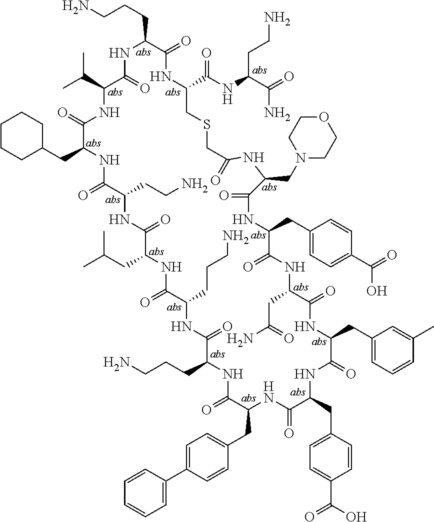 [SMILES: Cc1cccc(C[C@@H]2NC(=O)[C@H](CC(N)=O)NC(=O)[C@H](Cc3ccc(C(=O)O)cc3)NC(=O)[C@H](CN3CCOCC3)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](Cc3ccc(C(=O)O)cc3)NC2=O)c1]

Example 2727 was prepared on a 50 μmol scale. The yield of the product was 7.6 mg, and its estimated purity by LCMS analysis was 93.3%. Analysis condition B: Retention time=1.35 min; ESI-MS(+) m/z [M+2H]²⁺: 1053.3.

### Preparation of Example 2728

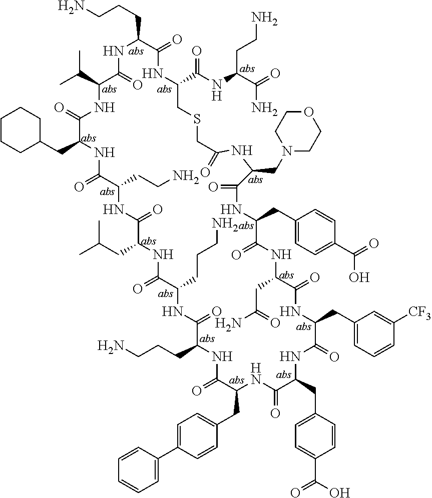 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(N)=O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCCN)NC(=O)[C@H](C(C)C)NC(=O)[C@H](CC2CCCCC2)NC(=O)[C@H](CCN)NC1=O]

Example 2728 was prepared on a 50 μmol scale. The yield of the product was 7.8 mg, and its estimated purity by LCMS analysis was 80.9%. Analysis condition B: Retention time=1.39 min; ESI-MS(+) m/z [M+2H]²⁺: 1080.2.

### Preparation of Example 2729

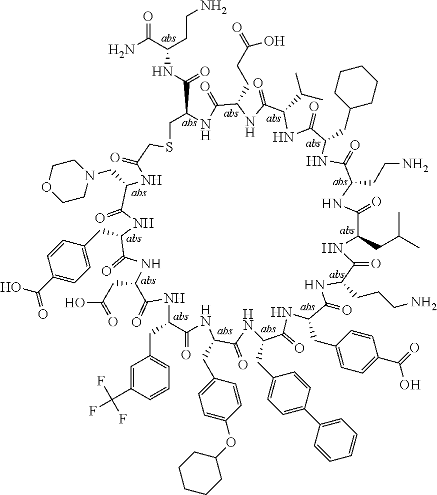 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(OC3CCCCC3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2729 was prepared on a 50 μmol scale. The yield of the product was 8.2 mg, and its estimated purity by LCMS analysis was 99.4%. Analysis condition B: Retention time=1.77 min; ESI-MS(+) m/z [M+2H]²⁺: 1153.9.

### Preparation of Example 2730

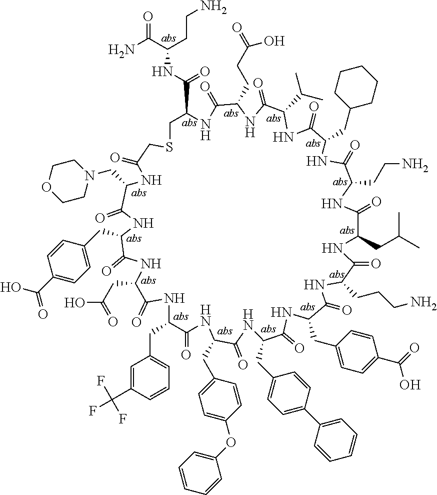 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(Oc3ccccc3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2730 was prepared on a 50 μmol scale. The yield of the product was 8.3 mg, and its estimated purity by LCMS analysis was 95.6%. Analysis condition A: Retention time=1.72 min; ESI-MS(+) m/z [M+2H]²⁺: 1151.1.

### Preparation of Example 2731

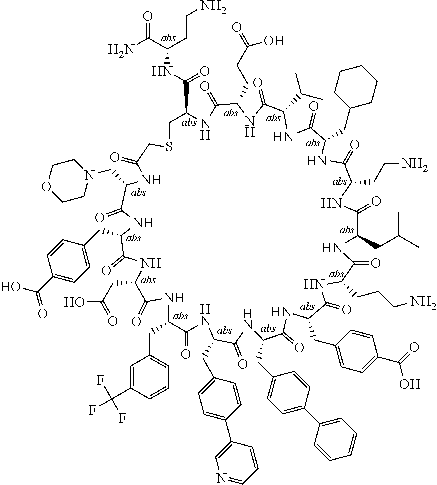 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(-c3cccnc3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2731 was prepared on a 50 μmol scale. The yield of the product was 6.5 mg, and its estimated purity by LCMS analysis was 100%. Analysis condition A: Retention time=1.55 min; ESI-MS(+) m/z [M+2H]²⁺: 1143.4.

### Preparation of Example 2732

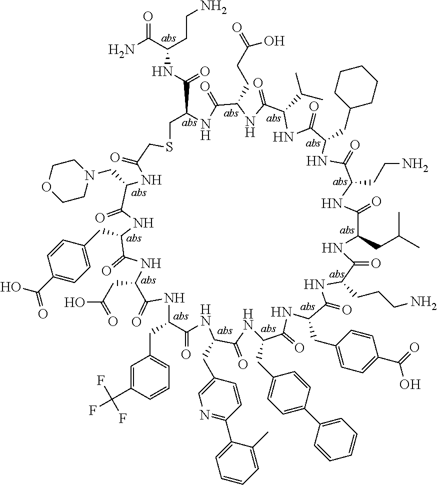 [SMILES: Cc1ccccc1-c1ccc(C[C@@H]2NC(=O)[C@H](Cc3cccc(C(F)(F)F)c3)NC(=O)[C@H](CC(=O)O)NC(=O)[C@H](Cc3ccc(C(=O)O)cc3)NC(=O)[C@H](CN3CCOCC3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cn1]

Example 2732 was prepared on a 50 μmol scale. The yield of the product was 9.4 mg, and its estimated purity by LCMS analysis was 95.8%. Analysis condition A: Retention time=1.63 min; ESI-MS(+) m/z [M+2H]²⁺: 1150.5.

### Preparation of Example 2733

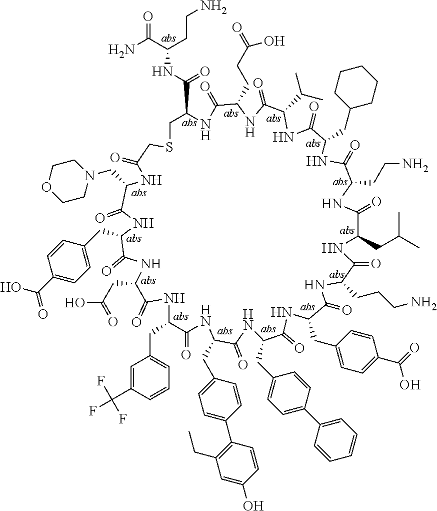 [SMILES: CCc1cc(O)ccc1-c1ccc(C[C@@H]2NC(=O)[C@H](Cc3cccc(C(F)(F)F)c3)NC(=O)[C@H](CC(=O)O)NC(=O)[C@H](Cc3ccc(C(=O)O)cc3)NC(=O)[C@H](CN3CCOCC3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Example 2733 was prepared on a 50 μmol scale. The yield of the product was 8.1 mg, and its estimated purity by LCMS analysis was 96.3%. Analysis condition A: Retention time=1.74 min; ESI-MS(+) m/z [M+2H]²⁺: 1164.9.

### Preparation of Example 2734

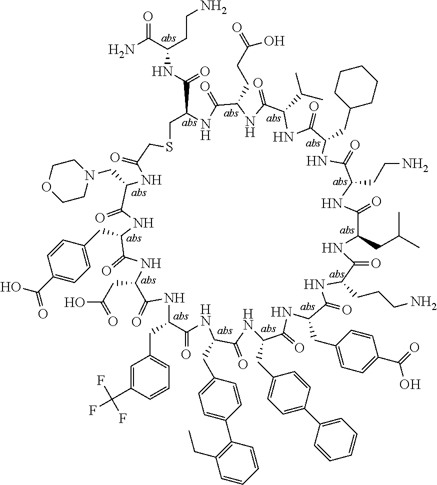 [SMILES: CCc1ccccc1-c1ccc(C[C@@H]2NC(=O)[C@H](Cc3cccc(C(F)(F)F)c3)NC(=O)[C@H](CC(=O)O)NC(=O)[C@H](Cc3ccc(C(=O)O)cc3)NC(=O)[C@H](CN3CCOCC3)NC(=O)CSC[C@@H](C(=O)N[C@@H](CCN)C(N)=O)NC(=O)[C@H](CCC(=O)O)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](Cc3ccc(C(=O)O)cc3)NC(=O)[C@H](Cc3ccc(-c4ccccc4)cc3)NC2=O)cc1]

Example 2734 was prepared on a 50 μmol scale. The yield of the product was 10.4 mg, and its estimated purity by LCMS analysis was 97.4%. Analysis condition A: Retention time=1.87 min; ESI-MS(+) m/z [M+2H]²⁺: 1156.9.

### Preparation of Example 2735

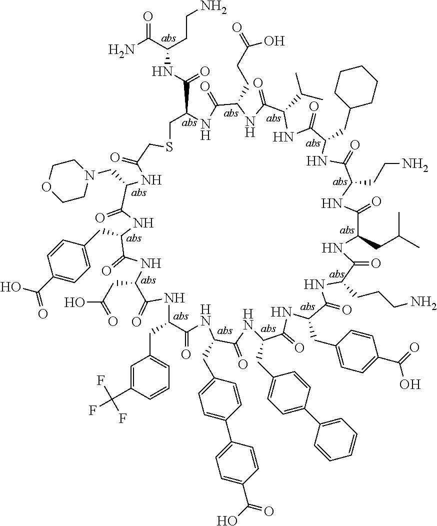 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(-c3ccc(C(=O)O)cc3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2735 was prepared on a 50 μmol scale. The yield of the product was 4.4 mg, and its estimated purity by LCMS analysis was 93.7%. Analysis condition B: Retention time=1.62 min; ESI-MS(+) m/z [M+2H]²⁺: 1164.7.

### Preparation of Example 2736

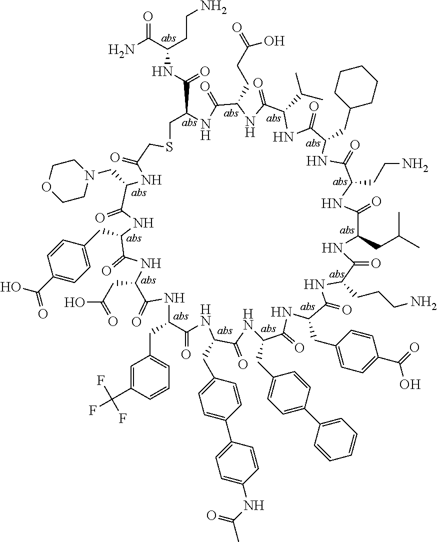 [SMILES: CC(=O)Nc1ccc(-c2ccc(C[C@@H]3NC(=O)[C@H](Cc4cccc(C(F)(F)F)c4)NC(=O)[C@H](CC(=O)O)NC(=O)[C@H](Cc4ccc(C(=O)O)cc4)NC(=O)[C@H](CN4CCOCC4)NC(=O)CSC[C@@H](C(=O)N[C@@H](CCN)C(N)=O)NC(=O)[C@H](CCC(=O)O)NC(=O)[C@H](C(C)C)NC(=O)[C@H](CC4CCCCC4)NC(=O)[C@H](CCN)NC(=O)[C@@H](CC(C)C)NC(=O)[C@H](CCCN)NC(=O)[C@H](Cc4ccc(C(=O)O)cc4)NC(=O)[C@H](Cc4ccc(-c5ccccc5)cc4)NC3=O)cc2)cc1]

Example 2736 was prepared on a 50 μmol scale. The yield of the product was 7.4 mg, and its estimated purity by LCMS analysis was 99%. Analysis condition A: Retention time=1.53 min; ESI-MS(+) m/z [M+2H]²⁺: 1171.4.

### Preparation of Example 2737

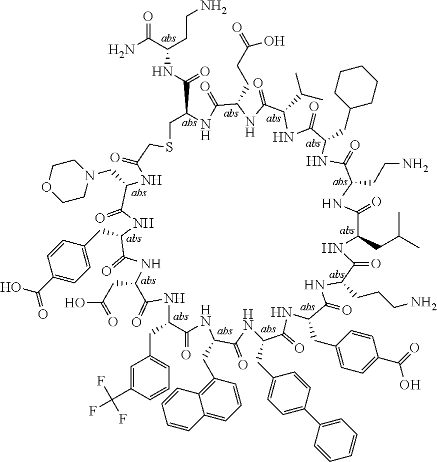 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2cccc3ccccc23)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2737 was prepared on a 50 μmol scale. The yield of the product was 6 mg, and its estimated purity by LCMS analysis was 91.4%. Analysis condition B: Retention time=1.7 min; ESI-MS(+) m/z [M+2H]²⁺: 1129.8.

### Preparation of Example 2738

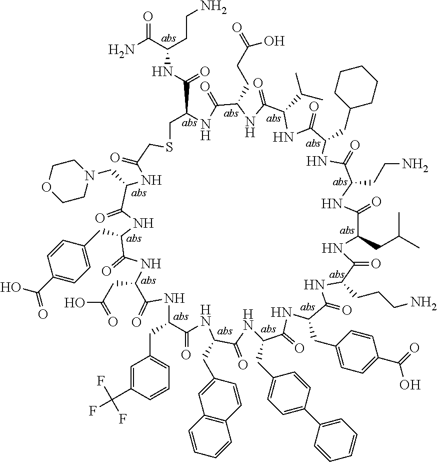 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc3ccccc3c2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2738 was prepared on a 50 μmol scale. The yield of the product was 13.5 mg, and its estimated purity by LCMS analysis was 95.2%. Analysis condition A: Retention time=1.69 min; ESI-MS(+) m/z [M+2H]²⁺: 1129.9.

### Preparation of Example 2739

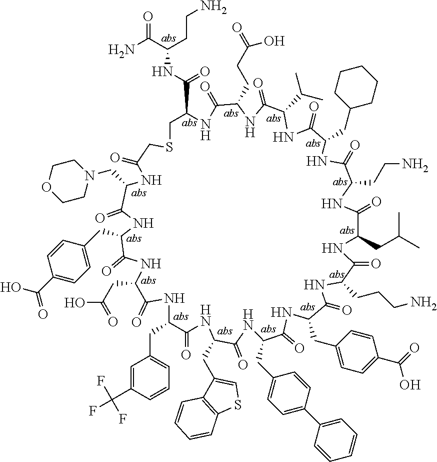 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2csc3ccccc23)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2739 was prepared on a 50 μmol scale. The yield of the product was 9.3 mg, and its estimated purity by LCMS analysis was 90%. Analysis condition B: Retention time=1.68 min; ESI-MS(+) m/z [M+2H]²⁺: 1132.7.

### Preparation of Example 2740

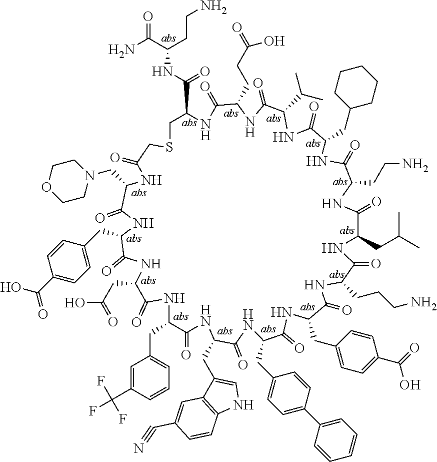 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2c[nH]c3ccc(C#N)cc23)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2740 was prepared on a 50 μmol scale. The yield of the product was 10.2 mg, and its estimated purity by LCMS analysis was 88.7%. Analysis condition B: Retention time=1.62 min; ESI-MS(+) m/z [M+2H]²⁺: 1136.8.

### Preparation of Example 2741

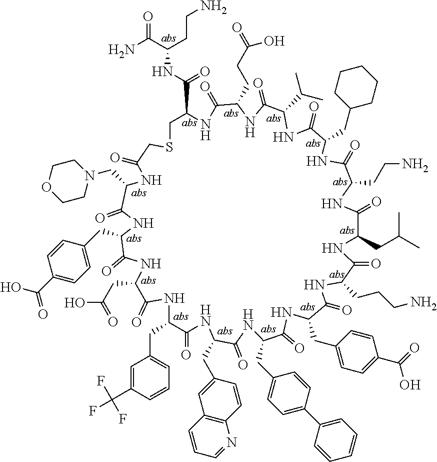 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc3ncccc3c2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2741 was prepared on a 50 μmol scale. The yield of the product was 12.5 mg, and its estimated purity by LCMS analysis was 86.9%. Analysis condition B: Retention time=1.51 min; ESI-MS(+) m/z [M+2H]²⁺: 1130.3.

### Preparation of Example 2742

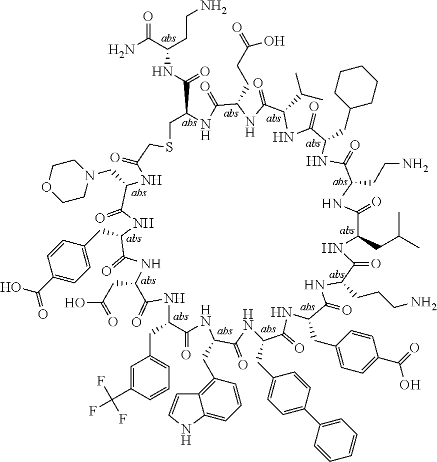 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2cccc3[nH]ccc23)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2742 was prepared on a 50 μmol scale. The yield of the product was 6.5 mg, and its estimated purity by LCMS analysis was 96.7%. Analysis condition A: Retention time=1.55 min; ESI-MS(+) m/z [M+2H]²⁺: 1124.4.

### Preparation of Example 2743

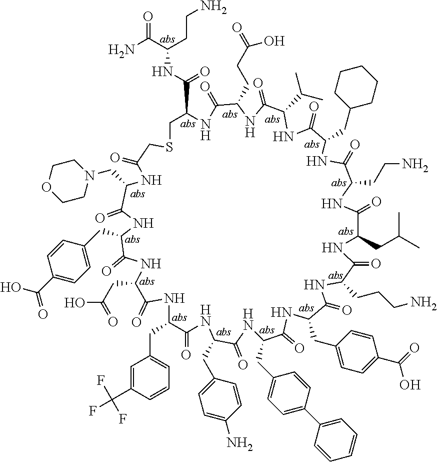 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(N)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2743 was prepared on a 50 μmol scale. The yield of the product was 8 mg, and its estimated purity by LCMS analysis was 88.9%. Analysis condition B: Retention time=1.49 min; ESI-MS(+) m/z [M+2H]²⁺: 1112.2.

### Preparation of Example 2744

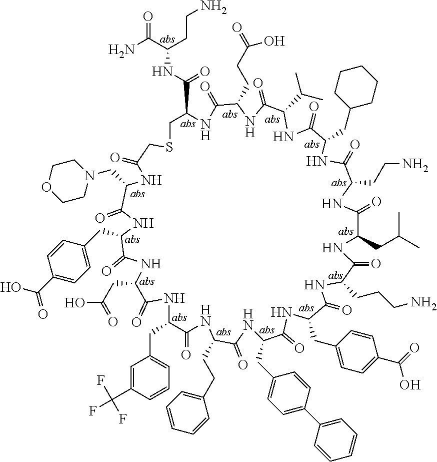 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](CCc2ccccc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2744 was prepared on a 50 μmol scale. The yield of the product was 10.4 mg, and its estimated purity by LCMS analysis was 98.6%. Analysis condition A: Retention time=1.65 min; ESI-MS(+) m/z [M+2H]²⁺: 1111.9.

### Preparation of Example 2745

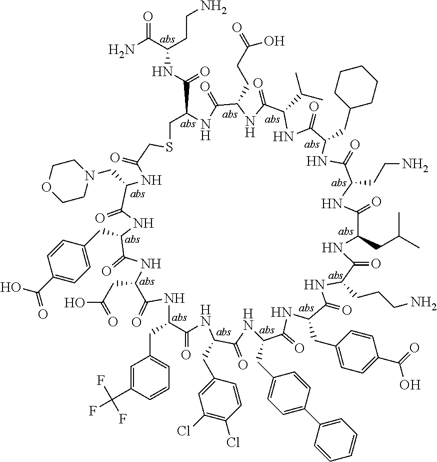 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(Cl)c(Cl)c2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2745 was prepared on a 50 μmol scale. The yield of the product was 8.8 mg, and its estimated purity by LCMS analysis was 95.3%. Analysis condition A: Retention time=1.72 min; ESI-MS(+) m/z [M+2H]²⁺: 1139.2.

### Preparation of Example 2746

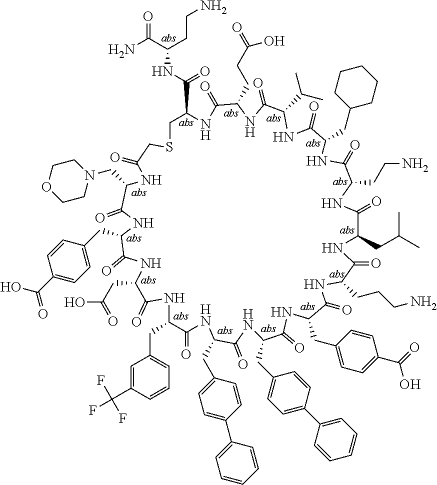 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(-c3ccccc3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2746 was prepared on a 50 μmol scale. The yield of the product was 9.4 mg, and its estimated purity by LCMS analysis was 98.8%. Analysis condition A: Retention time=1.75 min; ESI-MS(+) m/z [M+2H]²⁺: 1142.8.

### Preparation of Example 2747

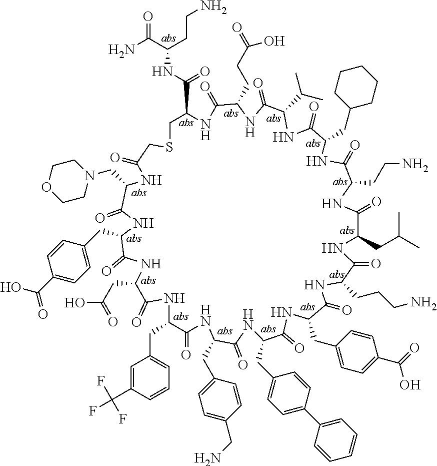 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(CN)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2747 was prepared on a 50 μmol scale. The yield of the product was 4.6 mg, and its estimated purity by LCMS analysis was 89.9%. Analysis condition B: Retention time=1.48 min; ESI-MS(+) m/z [M+2H]²⁺: 1119.4.

### Preparation of Example 2748

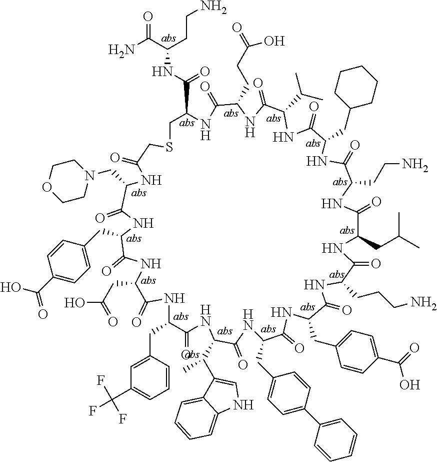 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H]([C@@H](C)c2c[nH]c3ccccc23)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2748 was prepared on a 50 μmol scale. The yield of the product was 4.1 mg, and its estimated purity by LCMS analysis was 94.9%. Analysis condition B: Retention time=1.61 min; ESI-MS(+) m/z [M+2H]²⁺: 1131.2.

### Preparation of Example 2749

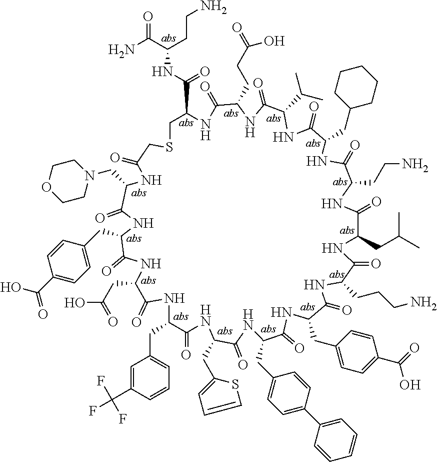 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2cccs2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2749 was prepared on a 50 μmol scale. The yield of the product was 4.8 mg, and its estimated purity by LCMS analysis was 95.7%. Analysis condition B: Retention time=1.63 min; ESI-MS(+) m/z [M+2H]²⁺: 1107.6.

### Preparation of Example 2750

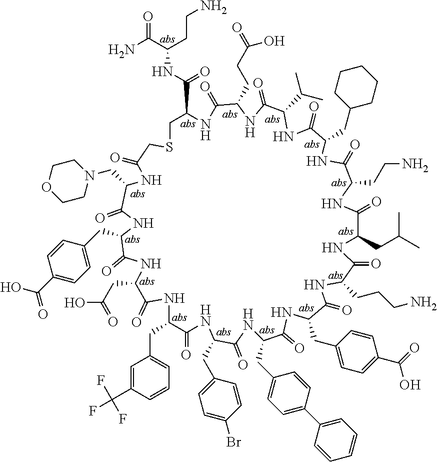 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(Br)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2750 was prepared on a 50 μmol scale. The yield of the product was 4.4 mg, and its estimated purity by LCMS analysis was 95.5%. Analysis condition B: Retention time=1.61 min; ESI-MS(+) m/z [M+2H]²⁺: 1144.6.

### Preparation of Example 2751

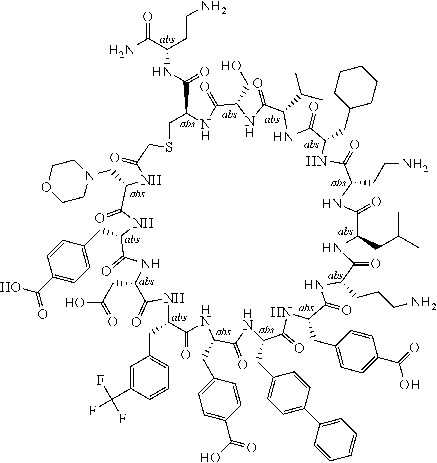 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)C)NC(=O)[C@H](CC2CCCCC2)NC(=O)[C@H](CCN)NC1=O]

Example 2751 was prepared on a 150 μmol scale. The yield of the product was 27.6 mg, and its estimated purity by LCMS analysis was 91.2%. Analysis condition A: Retention time=1.51 min; ESI-MS(+) m/z [M+2H]²⁺: 1105.9.

### Preparation of Example 2752

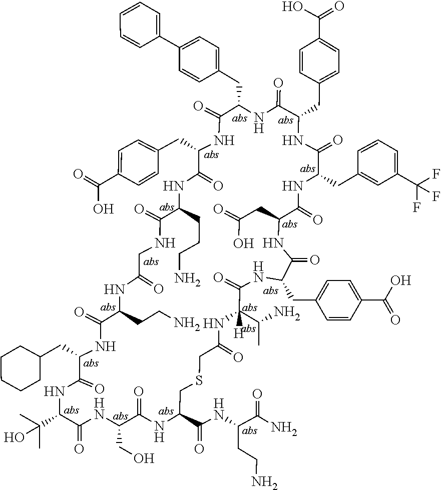 [SMILES: C[C@@H](N)[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)CNC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Example 2752 was prepared on a 50 μmol scale. The yield of the product was 1 mg, and its estimated purity by LCMS analysis was 87.9%. Analysis condition A: Retention time=1.31 min; ESI-MS(+) m/z [M+2H]²⁺: 1057.8.

### Preparation of Example 2753

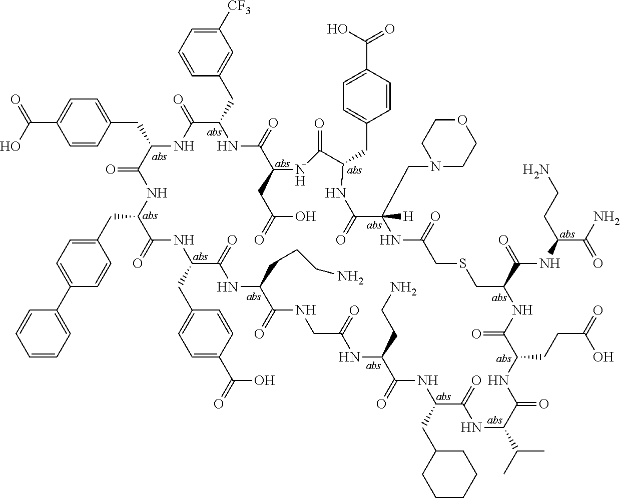 [SMILES: CC(C)[C@@H]1NC(=O)[C@H](CC2CCCCC2)NC(=O)[C@H](CCN)NC(=O)CNC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC1=O]

Example 2753 was prepared on a 50 μmol scale. The yield of the product was 10.7 mg, and its estimated purity by LCMS analysis was 85.4%. Analysis condition A: Retention time=1.23 min; ESI-MS(+) m/z [M+2H]²⁺: 1098.7.

### Preparation of Example 2754

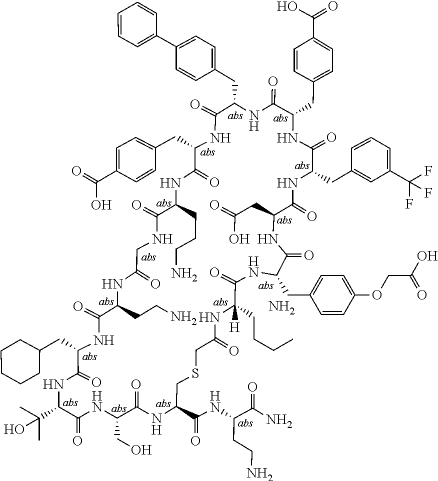

Example 2754 was prepared on a 50 μmol scale. The yield of the product was 12.4 mg, and its estimated purity by LCMS analysis was 85.8%. Analysis condition B: Retention time=1.55 min; ESI-MS(+) m/z [M+2H]²⁺: 1079.1.

### Preparation of Example 2755

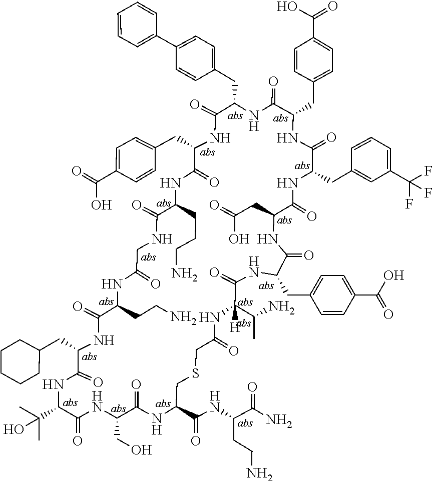 [SMILES: C[C@@H](N)[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)CNC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC1=O]

Example 2755 was prepared on a 50 μmol scale. The yield of the product was 3.1 mg, and its estimated purity by LCMS analysis was 100%. Analysis condition B: Retention time=1.45 min; ESI-MS(+) m/z [M+2H]²⁺: 1064.8.

### Preparation of Example 2756

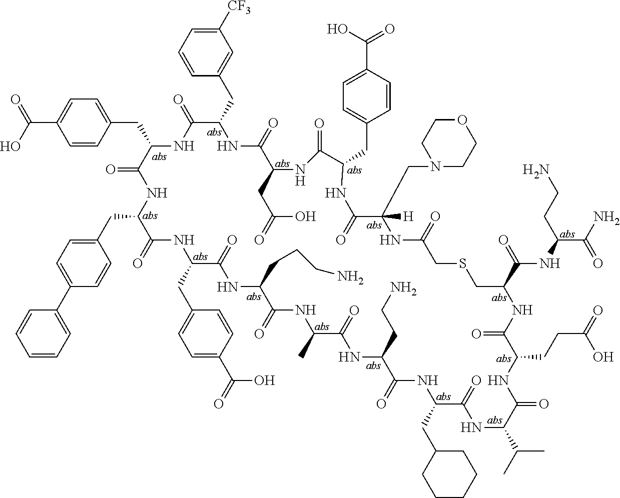 [SMILES: CC(C)[C@@H]1NC(=O)[C@H](CC2CCCCC2)NC(=O)[C@H](CCN)NC(=O)[C@@H](C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC1=O]

Example 2756 was prepared on a 50 μmol scale. The yield of the product was 3.8 mg, and its estimated purity by LCMS analysis was 95.8%. Analysis condition A: Retention time=1.25 min; ESI-MS(+) m/z [M+2H]²⁺: 1105.8.

### Preparation of Example 2757

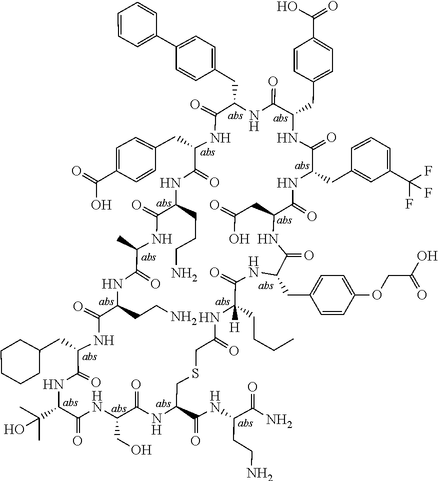 [SMILES: CCCC[C@@H]1NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](C)NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC1=O]

Example 2757 was prepared on a 50 μmol scale. The yield of the product was 13 mg, and its estimated purity by LCMS analysis was 100%. Analysis condition B: Retention time=1.59 min; ESI-MS(+) m/z [M+2H]²⁺: 1086.5.

### Preparation of Example 2758

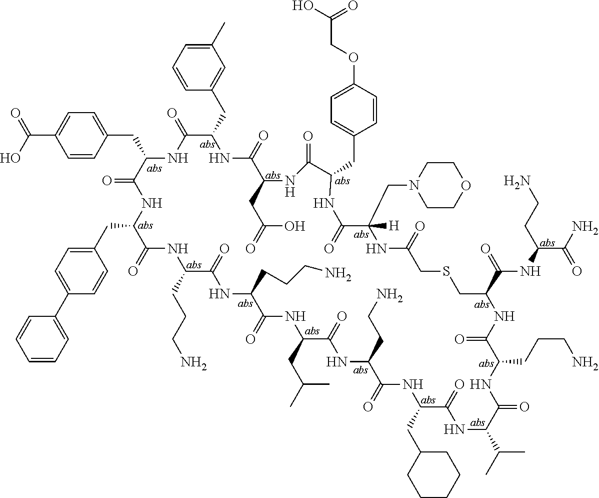 [SMILES: Cc1cccc(C[C@@H]2NC(=O)[C@H](CC(=O)O)NC(=O)[C@H](Cc3ccc(OCC(=O)O)cc3)NC(=O)[C@H](CN3CCOCC3)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](Cc3ccc(C(=O)O)cc3)NC2=O)c1]

Example 2758 was prepared on a 50 μmol scale. The yield of the product was 17.8 mg, and its estimated purity by LCMS analysis was 98.6%. Analysis condition A: Retention time=1.38 min; ESI-MS(+) m/z [M+2H]²⁺: 1069.2.

### Preparation of Example 2759

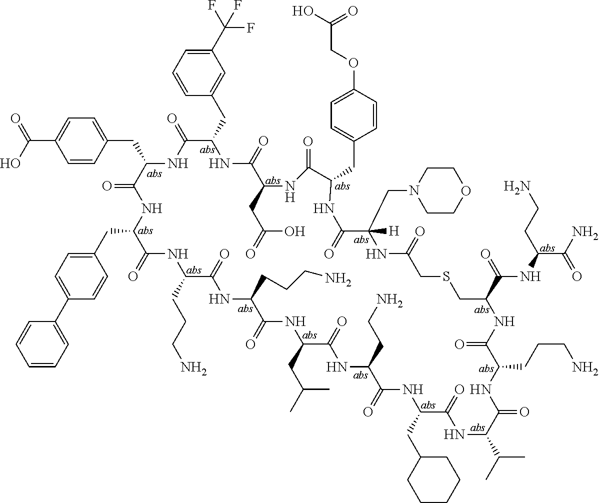 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CCCN)NC(=O)[C@H](C(C)C)NC(=O)[C@H](CC2CCCCC2)NC(=O)[C@H](CCN)NC1=O]

Example 2759 was prepared on a 50 μmol scale. The yield of the product was 22.4 mg, and its estimated purity by LCMS analysis was 86%. Analysis condition B: Retention time=1.41 min; ESI-MS(+) m/z [M+2H]²⁺: 1095.8.

### Preparation of Example 2760

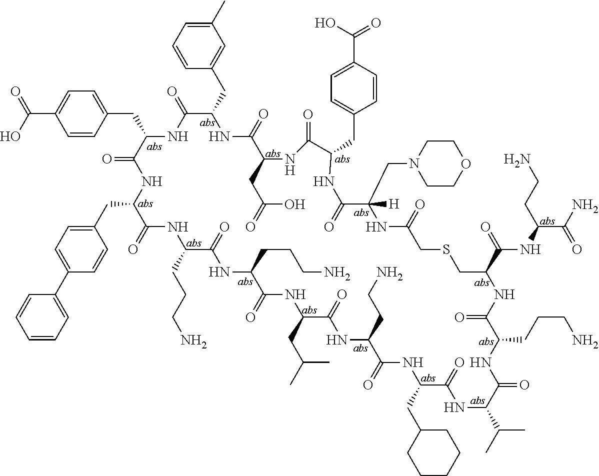 [SMILES: Cc1cccc(C[C@@H]2NC(=O)[C@H](CC(=O)O)NC(=O)[C@H](Cc3ccc(C(=O)O)cc3)NC(=O)[C@H](CN3CCOCC3)NC(=O)CSC[C@@H](C(=O)N[C@@H](CCN)C(N)=O)NC(=O)[C@H](CCCN)NC(=O)[C@H](C(C)C)NC(=O)[C@H](CC3CCCCC3)NC(=O)[C@H](CCN)NC(=O)[C@@H](CC(C)C)NC(=O)[C@H](CCCN)NC(=O)[C@H](CCCN)NC(=O)[C@H](Cc3ccc(-c4ccccc4)cc3)NC(=O)[C@H](Cc3ccc(C(=O)O)cc3)NC2=O)c1]

Example 2760 was prepared on a 50 μmol scale. The yield of the product was 14 mg, and its estimated purity by LCMS analysis was 85.2%. Analysis condition B: Retention time=1.32 min; ESI-MS(+) m/z [M+2H]²⁺: 1053.9.

### Preparation of Example 2761

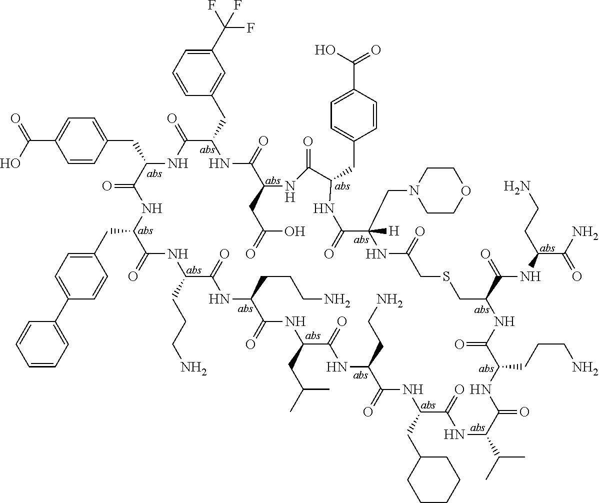 [SMILES: CC(C)C[C@H]1NC(=O)[C@H](CCCN)NC(=O)[C@H](CCCN)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCCN)NC(=O)[C@H](C(C)C)NC(=O)[C@H](CC2CCCCC2)NC(=O)[C@H](CCN)NC1=O]

Example 2761 was prepared on a 50 μmol scale. The yield of the product was 25.3 mg, and its estimated purity by LCMS analysis was 85.4%. Analysis condition B: Retention time=1.36 min; ESI-MS(+) m/z [M+2H]²⁺: 1081.2.

### Preparation of Example 2762

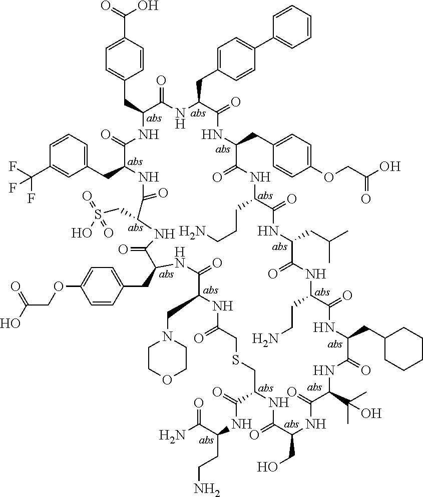 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@@H](CS(=O)(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2762 was prepared on a 50 μmol scale. The yield of the product was 9.9 mg, and its estimated purity by LCMS analysis was 85.7%. Analysis condition A: Retention time=1.47 min; ESI-MS(+) m/z [M+2H]²⁺: 1161.9.

### Preparation of Example 2763

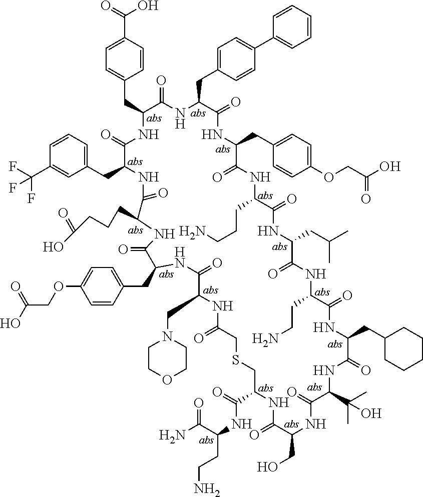 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CCCC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2763 was prepared on a 50 μmol scale. The yield of the product was 8.8 mg, and its estimated purity by LCMS analysis was 90.3%. Analysis condition A: Retention time=1.5 min; ESI-MS(+) m/z [M+2H]²⁺: 1157.9.

### Preparation of Example 2764

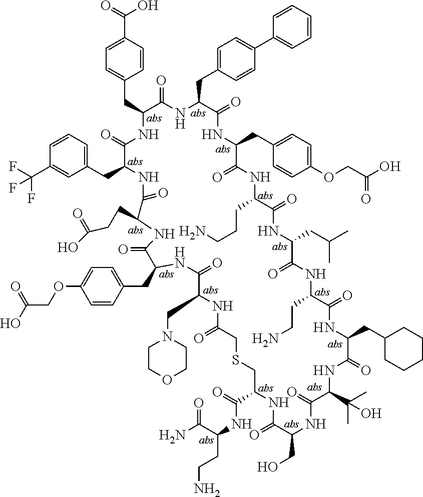 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CCC(=O)O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2764 was prepared on a 50 μmol scale. The yield of the product was 11.4 mg, and its estimated purity by LCMS analysis was 85.3%. Analysis condition B: Retention time=1.64 min; ESI-MS(+) m/z [M+2H]²⁺: 1151.

### Preparation of Example 2765

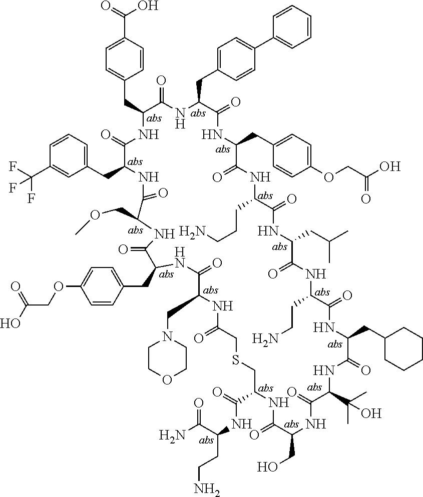 [SMILES: COC[C@@H]1NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC(=O)[C@@H](CC(C)C)NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC1=O]

Example 2765 was prepared on a 50 μmol scale. The yield of the product was 10.1 mg, and its estimated purity by LCMS analysis was 92.8%. Analysis condition B: Retention time=1.68 min; ESI-MS(+) m/z [M+2H]²⁺: 1137.2.

### Preparation of Example 2766

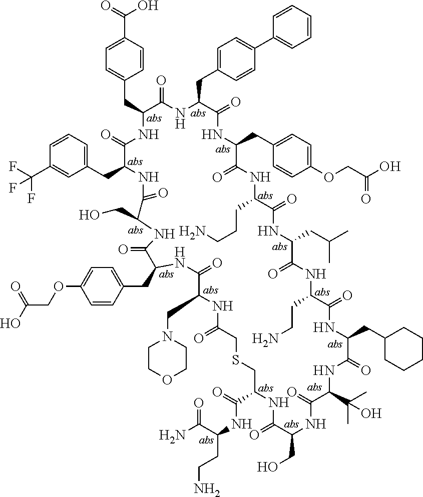 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CO)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2766 was prepared on a 50 μmol scale. The yield of the product was 6.2 mg, and its estimated purity by LCMS analysis was 96.2%. Analysis condition A: Retention time=1.61 min; ESI-MS(+) m/z [M+2H]²⁺: 1129.9.

### Preparation of Example 2767

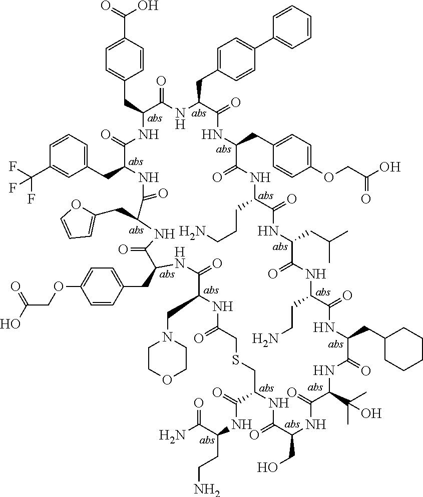 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](Cc2ccco2)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2767 was prepared on a 50 μmol scale. The yield of the product was 2.2 mg, and its estimated purity by LCMS analysis was 94.2%. Analysis condition A: Retention time=1.73 min; ESI-MS(+) m/z [M+2H]²⁺: 1154.8.

### Preparation of Example 2768

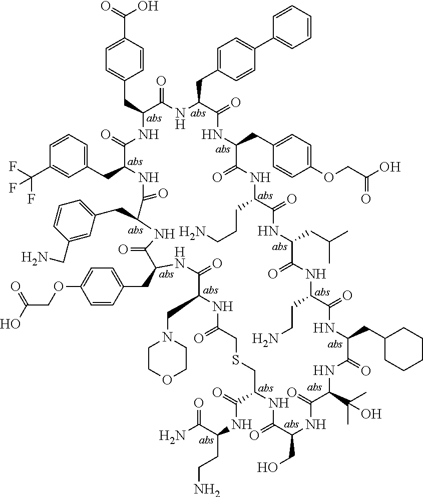 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](Cc2cccc(CN)c2)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2768 was prepared on a 50 μmol scale. The yield of the product was 2.4 mg, and its estimated purity by LCMS analysis was 95.5%. Analysis condition B: Retention time=1.59 min; ESI-MS(+) m/z [M+2H]²⁺: 1174.4.

### Preparation of Example 2769

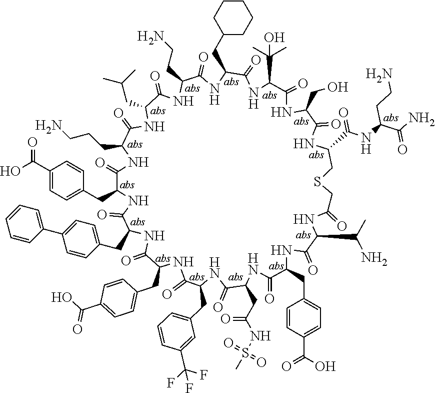 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)NS(C)(=O)=O)NC(=O)[C@H](Cc2ccc(C(=O)O)cc2)NC(=O)[C@H](C(C)N)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2769 was prepared on a 50 μmol scale. The yield of the product was 3 mg, and its estimated purity by LCMS analysis was 97.1%. Analysis condition A: Retention time=1.46 min; ESI-MS(+) m/z [M+2H]²⁺: 1124.7.

### Preparation of Example 2770

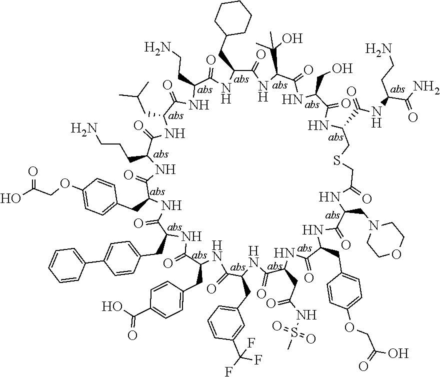 [SMILES: CC(C)C[C@H]1NC(=O)[C@H](CCCN)NC(=O)[C@H](Cc2ccc(OCC(=O)O)cc2)NC(=O)[C@H](Cc2ccc(-c3ccccc3)cc2)NC(=O)[C@H](Cc2ccc(C(=O)O)cc2)NC(=O)[C@H](Cc2cccc(C(F)(F)F)c2)NC(=O)[C@H](CC(=O)NS(C)(=O)=O)NC(=O)[C@H](Cc2ccc(OCC(=O)O)cc2)NC(=O)[C@H](CN2CCOCC2)NC(=O)CSC[C@@H](C(=O)N[C@@H](CCN)C(N)=O)NC(=O)[C@H](CO)NC(=O)[C@H](C(C)(C)O)NC(=O)[C@H](CC2CCCCC2)NC(=O)[C@H](CCN)NC1=O]

Example 2770 was prepared on a 50 μmol scale. The yield of the product was 17.2 mg, and its estimated purity by LCMS analysis was 95.1%. Analysis condition A: Retention time=1.43 min; ESI-MS(+) m/z [M+2H]²⁺: 1182.4.

### Preparation of Example 2771

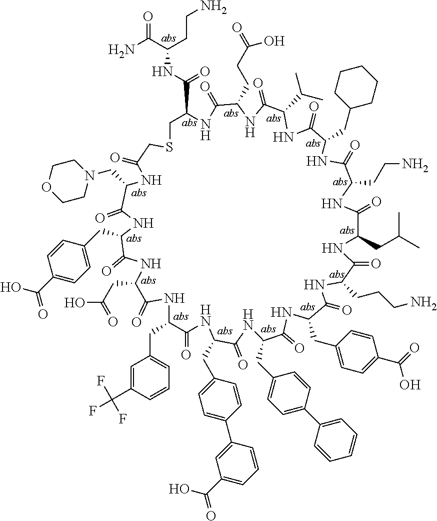 [SMILES: CC(C)C[C@H]1NC(=O)[C@H](CCCN)NC(=O)[C@H](Cc2ccc(C(=O)O)cc2)NC(=O)[C@H](Cc2ccc(-c3ccccc3)cc2)NC(=O)[C@H](Cc2ccc(-c3cccc(C(=O)O)c3)cc2)NC(=O)[C@H](Cc2cccc(C(F)(F)F)c2)NC(=O)[C@H](CC(=O)O)NC(=O)[C@H](Cc2ccc(C(=O)O)cc2)NC(=O)[C@H](CN2CCOCC2)NC(=O)CSC[C@@H](C(=O)N[C@@H](CCN)C(N)=O)NC(=O)[C@H](CCC(=O)O)NC(=O)[C@H](C(C)C)NC(=O)[C@H](CC2CCCCC2)NC(=O)[C@H](CCN)NC1=O]

Example 2771 was prepared on a 50 μmol scale. The yield of the product was 7.9 mg, and its estimated purity by LCMS analysis was 95.5%. Analysis condition A: Retention time=1.44 min; ESI-MS(+) m/z [M+2H]²⁺: 1164.8.

### Example 3. Jurkat-PD-1 Cell Binding High-Content Screening Assay (CBA)

Jurkat-PD-1 Cell Binding High-Content Screening Assay (CBA). Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×103 Jurkat-hPD-1 cells were seeded into 384 well plates in 20 μl of DMEM supplemented with 10% fetal calf serum. 100 nL of compound was added to cells followed by incubation at 37° C. for 2 h. Then, 5 μl of PE-labeled PD-L1-Ig (20 nM final), diluted in DMEM supplemented with 10% fetal calf serum. After 1 hour incubation, cells were fixed with 4% paraformaldehyde in dPBS containing 10 μg/ml Hoechst 33342 and then washed 3x in 100 μl dPBS. Data was collected and processed using a Cell Insight NXT High Content Imager and associated software.

Protein Sequence Information

hPDL1(18-239)-TVMV-mIgG1(221-447)-C225S

| | |
|---|---|
| 1 | AFTVTVPKDL YVVEYGSNMT IECKFPVEKQ LDLAALIVYW EMEDKNIIQF |
| 51 | VHGEEDLKVQ HSSYRQRARL LKDQLSLGNA ALQITDVKLQ DAGVYRCMIS |
| 101 | YGGADYKRIT VKVNAPYNKI NQRILVVDPV TSEHELTCQA EGYPKAEVIW |
| 151 | TSSDHQVLSG KTTTTNSKRE EKLFNVTSTL RINTTTNEIF YCTFRRLDPE |
| 201 | ENHTAELVIP ELPLAHPPNE RTGSPGGGGG RETVRFQGGT GDAVPRDSGC |
| 251 | KPCICTVPEV SSVFIFPPKP KDVLTITLTP KVTCVVVDIS KDDPEVQFSW |
| 301 | FVDDVEVHTA QTQPREEQFN STFRSVSELP IMHQDWLNGK EFKCRVNSAA |
| 351 | FPAPIEKTIS KTKGRPKAPQ VYTIPPPKEQ MAKDKVSLTC MITDFFPEDI |
| 401 | TVEWQWNGQP AENYKNTQPI MDTDGSYFVY SKLNVQKSNW EAGNTFTCSV |
| 451 | LHEGLHNHHT EKSLSHSPGK |

Jurkat HPDL1 PD1 IC₅₀ (μM) is presented in Table 3.

TABLE 3
Jurkat HPDL1 PD1 IC₅₀ (μM)

| Compound | Jurkat HPD1_PDL1 |
|---|---|
| Number | IC₅₀ (μM) |
| 1000 | 0.2507 |
| 1001 | 0.1397 |
| 1002 | 0.0172 |
| 1003 | 0.6138 |
| 1004 | 0.0532 |
| 1005 | 0.0358 |
| 1006 | 0.0981 |
| 1007 | 0.0453 |
| 1008 | 0.0599 |
| 1009 | 0.0529 |
| 1010 | 0.0996 |
| 1011 | 0.0671 |
| 1012 | 0.0436 |
| 1013 | 0.1290 |
| 1014 | 0.0167 |
| 1015 | 0.0238 |
| 1016 | 0.0319 |
| 1017 | 0.0203 |
| 1018 | 0.0207 |
| 1019 | 0.0206 |
| 1020 | 0.0023 |
| 1021 | 0.0268 |
| 1022 | 0.0177 |
| 1023 | 0.0046 |
| 1024 | 0.0373 |
| 1025 | 0.0110 |
| 1026 | 0.0108 |
| 1027 | 0.0195 |
| 1028 | 0.0143 |
| 1029 | 0.0054 |
| 1030 | 0.0108 |
| 1031 | 0.0874 |
| 1032 | 0.0155 |
| 1033 | 0.0294 |
| 1034 | 0.0990 |
| 1035 | 0.0437 |
| 1036 | 0.0264 |
| 1037 | 0.0539 |
| 1038 | 0.0070 |
| 1039 | 0.0121 |
| 1040 | 0.0243 |
| 1041 | 0.0424 |
| 1042 | 0.0130 |
| 1043 | 0.0319 |
| 1044 | 0.0261 |
| 1045 | 0.0284 |
| 1046 | 0.0140 |
| 1047 | 0.0067 |
| 1048 | 0.0137 |
| 1049 | 0.0072 |
| 1050 | 0.0099 |
| 1051 | 0.0052 |
| 1052 | 0.0118 |
| 1053 | 0.0137 |
| 1054 | 0.0090 |
| 1055 | 0.0058 |
| 1056 | 0.0086 |
| 1057 | 0.1286 |
| 1058 | 0.0181 |
| 1059 | 0.0285 |
| 1060 | 0.0164 |
| 1061 | 0.0088 |
| 1062 | 0.0124 |
| 1063 | 0.0198 |
| 1064 | 0.0513 |
| 1065 | 0.0128 |
| 1066 | 0.0422 |
| 1067 | 0.0222 |
| 1068 | 0.0174 |
| 1069 | 0.0139 |
| 1070 | 0.0388 |
| 1071 | 0.0080 |
| 1072 | 0.0271 |
| 1073 | 0.0100 |
| 1074 | 0.1245 |
| 1075 | 0.0501 |
| 1076 | 0.0687 |
| 1077 | 0.0467 |
| 1078 | 0.0402 |
| 1079 | 0.0295 |
| 1080 | 0.0471 |
| 1081 | 0.0286 |
| 1082 | 0.0415 |
| 1083 | 0.0168 |
| 1084 | 0.0289 |
| 1085 | 0.0223 |
| 1086 | 0.0205 |
| 1087 | 0.0366 |
| 1088 | 0.0237 |
| 1089 | 0.0267 |
| 1090 | 0.0280 |
| 1091 | 0.0218 |
| 1092 | 0.2292 |
| 1093 | 0.1198 |
| 1094 | 0.1262 |
| 1095 | 0.1457 |
| 1096 | 0.0688 |
| 1097 | 0.1319 |
| 1098 | 0.1152 |
| 1099 | 0.0845 |
| 1100 | 0.0414 |
| 1101 | 0.1272 |
| 1102 | 0.0711 |
| 1103 | 0.1116 |
| 1104 | 0.0436 |
| 1105 | 0.1017 |
| 1106 | 0.0667 |
| 1107 | 0.3104 |
| 1108 | 0.0425 |
| 1109 | 0.0360 |
| 1110 | 0.0356 |
| 1111 | 0.0379 |
| 1112 | 0.0171 |
| 1113 | 0.0156 |
| 1114 | 0.0316 |
| 1115 | 0.0796 |
| 1116 | 0.0385 |
| 1117 | 0.0393 |
| 1118 | 0.0231 |
| 1119 | 0.0133 |
| 1120 | 0.0115 |
| 1121 | 0.0209 |
| 1122 | 0.0162 |
| 1123 | 0.0148 |
| 1124 | 0.0243 |
| 1125 | 0.0216 |
| 1126 | 0.0239 |
| 1127 | 0.0150 |
| 1128 | 0.0176 |
| 1129 | 0.0197 |
| 1130 | 0.0195 |
| 1131 | 0.0248 |
| 1132 | 0.0207 |
| 1133 | 0.0331 |
| 1134 | 0.0321 |
| 1135 | 0.0130 |
| 1136 | 0.0508 |
| 1137 | 0.0470 |
| 1138 | 0.0193 |
| 1139 | 0.0444 |
| 1140 | 0.0577 |
| 1141 | 0.0306 |
| 1142 | 0.0196 |
| 1143 | 0.0158 |
| 1144 | 0.0192 |
| 1145 | 0.0087 |
| 1146 | 0.0423 |
| 1147 | 0.0227 |
| 1148 | 0.0510 |
| 1149 | 0.0470 |
| 1150 | 0.0489 |
| 1151 | 0.0709 |
| 1152 | 0.0647 |
| 1153 | 0.0089 |
| 1154 | 0.0092 |
| 1155 | 0.0077 |
| 1156 | 0.0207 |
| 1157 | 0.0106 |
| 1158 | 0.0731 |
| 1159 | 0.0081 |
| 1160 | 0.0313 |
| 1161 | 0.0161 |
| 1162 | 0.3274 |
| 1163 | 0.0261 |
| 1164 | 0.0961 |
| 1165 | 0.0146 |
| 1166 | 0.0583 |
| 1167 | 0.0170 |
| 1168 | 0.0263 |
| 1169 | 0.0207 |
| 1170 | 0.0108 |
| 1171 | 0.0264 |
| 1172 | 0.0064 |
| 1173 | 0.0097 |
| 1174 | 0.0268 |
| 1175 | 0.0820 |
| 1176 | 0.0757 |
| 1177 | 0.0789 |
| 1178 | 0.0041 |
| 1179 | 0.0058 |
| 1180 | 0.0089 |
| 1181 | 0.0120 |
| 1182 | 0.0082 |
| 1183 | 0.0061 |
| 1184 | 0.0062 |
| 1185 | 0.0074 |
| 1186 | 0.0058 |
| 1187 | 0.0107 |
| 1188 | 0.0079 |
| 1189 | 0.0177 |
| 1190 | 0.0045 |
| 1191 | 0.0100 |
| 1192 | 0.0071 |
| 1193 | 0.0077 |
| 1194 | 0.0047 |
| 1195 | 0.0138 |
| 1196 | 0.0082 |
| 1197 | 0.0120 |
| 1198 | 0.0156 |
| 1199 | 0.0041 |
| 1200 | 0.0054 |
| 1201 | 0.0034 |
| 1202 | 0.0098 |
| 1203 | 0.0474 |
| 1204 | 0.0057 |
| 1205 | 0.0107 |
| 1206 | 0.0089 |
| 1207 | 0.0068 |
| 1208 | 0.0027 |
| 1209 | 0.0099 |
| 1210 | 0.0115 |
| 1211 | 0.0089 |
| 1212 | 0.0060 |
| 1213 | 0.0208 |
| 1214 | 0.0141 |
| 1215 | 0.0470 |
| 1216 | 0.0222 |
| 1217 | 0.0254 |
| 1218 | 0.0056 |
| 1219 | 0.0054 |
| 1220 | 0.0051 |
| 1221 | 0.0030 |
| 1222 | 0.0147 |
| 1223 | 0.0061 |
| 1224 | 0.0113 |
| 1225 | 0.0062 |
| 1226 | 0.0322 |
| 1227 | 0.0298 |
| 1228 | 0.0175 |
| 1229 | 0.0145 |
| 1230 | 0.0181 |
| 1231 | 0.0271 |
| 1232 | 0.0149 |
| 1233 | 0.0345 |
| 1234 | 0.0366 |
| 1235 | 0.0210 |
| 1236 | 0.0174 |
| 1237 | 0.0294 |
| 1238 | 0.0150 |
| 1239 | 0.0143 |
| 1240 | 0.1192 |
| 1241 | 0.0278 |
| 1242 | 0.0289 |
| 1243 | 0.0429 |
| 1244 | 0.0223 |
| 1245 | 0.0208 |
| 1246 | 0.0844 |
| 1247 | 0.0197 |
| 1248 | 0.0124 |
| 1249 | 0.0046 |
| 1250 | 0.0069 |
| 1251 | 0.0079 |
| 1252 | 0.0215 |
| 1253 | 0.0068 |
| 1254 | 0.0074 |
| 1255 | 0.0145 |
| 1256 | 0.0067 |
| 1257 | 0.0056 |
| 1258 | 0.0080 |
| 1259 | 0.0054 |
| 1260 | 0.0074 |
| 1261 | 0.0131 |
| 1262 | 0.0046 |
| 1263 | 0.0062 |
| 1264 | 0.0045 |
| 1265 | 0.0077 |
| 1266 | 0.0296 |
| 1267 | 0.0999 |
| 1268 | 0.0246 |
| 1269 | 0.0404 |
| 1270 | 0.0325 |
| 1271 | 0.0086 |
| 1272 | 0.0110 |
| 1273 | 0.0548 |
| 1274 | 0.0384 |
| 1275 | 0.0138 |
| 1276 | 0.0081 |
| 1277 | 0.0086 |
| 1278 | 0.0273 |
| 1279 | 0.0136 |
| 1280 | 0.0097 |
| 1281 | 0.0089 |
| 1282 | 0.0118 |
| 1283 | 0.0245 |
| 1284 | 0.0258 |
| 1285 | 0.0426 |
| 1286 | 0.0503 |
| 1287 | 0.0351 |
| 1288 | 0.0389 |
| 1289 | 0.0110 |
| 1290 | 0.0506 |
| 1291 | 0.0121 |
| 1292 | 0.0072 |
| 1293 | 0.0106 |
| 1294 | 0.0047 |
| 1295 | 0.0098 |
| 1296 | 0.0135 |
| 1297 | 0.0094 |
| 1298 | 0.0073 |
| 1299 | 0.1207 |
| 1300 | 0.0050 |
| 1301 | 0.0059 |
| 1302 | 0.0061 |
| 1303 | 0.0048 |
| 1304 | 0.0265 |
| 1305 | 0.0117 |
| 1306 | 0.0146 |
| 1307 | 0.0374 |
| 1308 | 0.0126 |
| 1309 | 0.0484 |
| 1310 | 0.0120 |
| 1311 | 0.0160 |
| 1312 | 0.0040 |
| 1313 | 0.0157 |
| 1314 | 0.0090 |
| 1315 | 0.0186 |
| 1316 | 0.0091 |
| 1317 | 0.9124 |
| 1318 | 0.0106 |
| 1319 | 0.0497 |
| 1320 | 0.0527 |
| 1321 | 0.0786 |
| 1322 | 0.0213 |
| 1323 | 0.0294 |
| 1324 | 0.0155 |
| 1325 | 0.0031 |
| 1326 | 0.0121 |
| 1327 | 0.0316 |
| 1328 | 0.0345 |
| 1329 | 0.0068 |
| 1330 | 0.0128 |
| 1331 | 0.0060 |
| 1332 | 0.0117 |
| 1333 | 0.0069 |
| 1334 | 0.0269 |
| 1335 | 0.0209 |
| 1336 | 0.0212 |
| 1337 | 0.0270 |
| 1338 | 0.0380 |
| 1339 | 0.0520 |
| 1340 | 0.0217 |
| 1341 | 0.0528 |
| 1342 | 0.0322 |
| 1343 | 0.0299 |
| 1344 | 0.0310 |
| 1345 | 0.2356 |
| 1346 | 0.0958 |
| 1347 | 0.0264 |
| 1348 | 0.0400 |
| 1349 | 0.0549 |
| 1350 | 0.1620 |
| 1351 | 0.1208 |
| 1352 | 0.1096 |
| 1353 | 0.0109 |
| 1354 | 0.0139 |
| 1355 | 0.0082 |
| 1356 | 0.0055 |
| 1357 | 0.0048 |
| 1358 | 0.0073 |
| 1359 | 0.0079 |
| 1360 | 0.0108 |
| 1361 | 0.0149 |
| 1362 | 0.0529 |
| 1363 | 0.0345 |
| 1364 | 0.0576 |
| 1365 | 0.0164 |
| 1366 | 0.8581 |
| 1367 | 0.0217 |
| 1368 | 0.0071 |
| 1369 | 0.0199 |
| 1370 | 0.0064 |
| 1371 | 0.0150 |
| 1372 | 0.0126 |
| 1373 | 0.0111 |
| 1374 | 0.0098 |
| 1375 | 0.0160 |
| 1376 | 0.0072 |
| 1377 | 0.0089 |
| 1378 | 0.0137 |
| 1379 | 0.0330 |
| 1380 | 0.0168 |
| 1381 | 0.0133 |
| 1382 | 0.0140 |
| 1383 | 0.0191 |
| 1384 | 0.0110 |
| 1385 | 0.0061 |
| 1386 | 0.0108 |
| 1387 | 0.0249 |
| 1388 | 0.0260 |
| 1389 | 0.0085 |
| 1390 | 0.0165 |
| 1391 | 0.0050 |
| 1392 | 0.0781 |
| 1393 | 0.0253 |
| 1394 | 0.0076 |
| 1395 | 0.0101 |
| 1396 | 0.0214 |
| 1397 | 0.0553 |
| 1398 | 0.0237 |
| 1399 | 0.0161 |
| 1400 | 0.0291 |
| 1401 | 0.0220 |
| 1402 | 0.0099 |
| 1403 | 0.0059 |
| 1404 | 0.0051 |
| 1405 | 0.0074 |
| 1406 | 0.0159 |
| 1407 | 0.0079 |
| 1408 | 0.0082 |
| 1409 | 0.0168 |
| 1410 | 0.0073 |
| 1411 | 0.0095 |
| 1412 | 0.0269 |
| 1413 | 0.0088 |
| 1414 | 0.0099 |
| 1415 | 0.0138 |
| 1416 | 0.0085 |
| 1417 | 0.0070 |
| 1418 | 0.0081 |
| 1419 | 0.0072 |
| 1420 | 0.0075 |
| 1421 | 0.0112 |
| 1422 | 0.0101 |
| 1423 | 0.0099 |
| 1424 | 0.0147 |
| 1425 | 0.0168 |
| 1426 | 0.0233 |
| 1427 | 0.0097 |
| 1428 | 0.0184 |
| 1429 | 0.0095 |
| 1430 | 0.0108 |
| 1431 | 0.0116 |
| 1432 | 0.0174 |
| 1433 | 0.0405 |
| 1434 | 0.0359 |
| 1435 | 0.0531 |
| 1436 | 0.0228 |
| 1437 | 0.0166 |
| 1438 | 0.0304 |
| 1439 | 0.0055 |
| 1440 | 0.0868 |
| 1441 | 0.0134 |
| 1442 | 0.0159 |
| 1443 | 0.0118 |
| 1444 | 0.0057 |
| 1445 | 0.0195 |
| 1446 | 0.0645 |
| 1447 | 0.0298 |
| 1448 | 0.0269 |
| 1449 | 0.0329 |
| 1450 | 0.0076 |
| 1451 | 0.0103 |
| 1452 | 0.0252 |
| 1453 | 0.0097 |
| 1454 | 0.0119 |
| 1455 | 0.0083 |
| 1456 | 0.0061 |
| 1457 | 0.0198 |
| 1458 | 0.0179 |
| 1459 | 0.0177 |
| 1460 | 0.0112 |
| 1461 | 0.0136 |
| 1462 | 0.0168 |
| 1463 | 0.0244 |
| 1464 | 0.0258 |
| 1465 | 0.0274 |
| 1466 | 0.0163 |
| 1467 | 0.1120 |
| 1468 | 0.0735 |
| 1469 | 0.0296 |
| 1470 | 0.0546 |
| 1471 | 0.3052 |
| 1472 | 0.1282 |
| 1473 | 0.8012 |
| 1474 | 0.0686 |
| 1475 | 0.1202 |
| 1476 | 0.0330 |
| 1477 | 0.1435 |
| 1478 | 0.1205 |
| 1479 | 0.3475 |
| 1480 | 0.1504 |
| 1481 | 0.0672 |
| 1482 | 0.1039 |
| 1483 | 0.0348 |
| 1484 | 0.0123 |
| 1485 | 0.0155 |
| 1486 | 0.0437 |
| 1487 | 0.2683 |
| 1488 | 0.0106 |
| 1489 | 0.0136 |
| 1490 | 0.0202 |
| 1491 | 0.0226 |
| 1492 | 0.0274 |
| 1493 | 0.0108 |
| 1494 | 0.0138 |
| 1495 | 0.0058 |
| 1496 | 0.0179 |
| 1497 | 0.3436 |
| 1498 | 0.3663 |
| 1499 | 0.6631 |
| 1500 | 0.2376 |
| 1501 | 0.0107 |
| 1502 | 0.0168 |
| 1503 | 0.0428 |
| 1504 | 0.0052 |
| 1505 | 0.0237 |
| 1506 | 0.0567 |
| 1507 | 0.1538 |
| 1508 | 0.1627 |
| 1509 | 0.4134 |
| 1510 | 0.0257 |
| 1511 | 0.7370 |
| 1512 | 0.0229 |
| 1513 | 0.0257 |
| 1514 | 0.0348 |
| 1515 | 0.0288 |
| 1516 | 0.0300 |
| 1517 | 0.0342 |
| 1518 | 0.0187 |
| 1519 | 0.0077 |
| 1520 | 0.0105 |
| 1521 | 0.0177 |
| 1522 | 0.0233 |
| 1523 | 0.0143 |
| 1524 | 0.0092 |
| 1525 | 0.0154 |
| 1526 | 0.1935 |
| 1527 | 0.1027 |
| 1528 | 0.1003 |
| 1529 | 0.0435 |
| 1530 | 0.0949 |
| 1531 | 0.7069 |
| 1532 | 0.1108 |
| 1533 | 0.0413 |
| 1534 | 0.0831 |
| 1535 | 0.1275 |
| 1536 | 0.0593 |
| 1537 | 0.1009 |
| 1538 | 0.1352 |
| 1539 | 0.0857 |
| 1540 | 0.0874 |
| 1541 | 0.0166 |
| 1542 | 0.0186 |
| 1543 | 0.0277 |
| 1544 | 0.0105 |
| 1545 | 0.0178 |
| 1546 | 0.0090 |
| 1547 | 0.0159 |
| 1548 | 0.0184 |
| 1549 | 0.0109 |
| 1550 | 0.0945 |
| 1551 | 0.0156 |
| 1552 | 0.0218 |
| 1553 | 0.0269 |
| 1554 | 0.0962 |
| 1555 | 0.0433 |
| 1556 | 0.0909 |
| 1557 | 0.0379 |
| 1558 | 0.0158 |
| 1559 | 0.0107 |
| 1560 | 0.0169 |
| 1561 | 0.0162 |
| 1562 | 0.0202 |
| 1563 | 0.0107 |
| 1564 | 0.0361 |
| 1565 | 0.0227 |
| 1566 | 0.0105 |
| 1567 | 0.0258 |
| 1568 | 0.0245 |
| 1569 | 0.0164 |
| 1570 | 0.0184 |
| 1571 | 0.0169 |
| 1572 | 0.0172 |
| 1573 | 0.0181 |
| 1574 | 0.0304 |
| 1575 | 0.0166 |
| 1576 | 0.0303 |
| 1577 | 0.0121 |
| 1578 | 0.0350 |
| 1579 | 0.0178 |
| 1580 | 0.0494 |
| 1581 | 0.0224 |
| 1582 | 0.0362 |
| 1583 | 0.0200 |
| 1584 | 0.0241 |
| 1585 | 0.0504 |
| 1586 | 0.0177 |
| 1587 | 0.0260 |
| 1588 | 0.0254 |
| 1589 | 0.0164 |
| 1590 | 0.0090 |
| 1591 | 0.0179 |
| 1592 | 0.0315 |
| 1593 | 0.0230 |
| 1594 | 0.0194 |
| 1595 | 0.0171 |
| 1596 | 0.0081 |
| 1597 | 0.0202 |
| 1598 | 0.0099 |
| 1599 | 0.0119 |
| 1600 | 0.0096 |
| 1601 | 0.0327 |
| 1602 | 0.0238 |
| 1603 | 0.0116 |
| 1604 | 0.0458 |
| 1605 | 0.0231 |
| 1606 | 0.0129 |
| 1607 | 0.0146 |
| 1608 | 0.0322 |
| 1609 | 0.0153 |
| 1610 | 0.0215 |
| 1611 | 0.0096 |
| 1612 | 0.0063 |
| 1613 | 0.0207 |
| 1614 | 0.0144 |
| 1615 | 0.0159 |
| 1616 | 0.0093 |
| 1617 | 0.0085 |
| 1618 | 0.0227 |
| 1619 | 0.0146 |
| 1620 | 0.0050 |
| 1621 | 0.0367 |
| 1622 | 0.0360 |
| 1623 | 0.0270 |
| 1624 | 0.0269 |
| 1625 | 0.0350 |
| 1626 | 0.0519 |
| 1627 | 0.0193 |
| 1628 | 0.0249 |
| 1629 | 0.0199 |
| 1630 | 0.0483 |
| 1631 | 0.0441 |
| 1632 | 0.0156 |
| 1633 | 0.0202 |
| 1634 | 0.0203 |
| 1635 | 0.0181 |
| 1636 | 0.0197 |
| 1637 | 0.0327 |
| 1638 | 0.0286 |
| 1639 | 0.0358 |
| 1640 | 0.0257 |
| 1641 | 0.0371 |
| 1642 | 0.0304 |
| 1643 | 0.0145 |
| 1644 | 0.0167 |
| 1645 | 0.0203 |
| 1646 | 0.0281 |
| 1647 | 0.0509 |
| 1648 | 0.0970 |
| 1649 | 0.0345 |
| 1650 | 0.0242 |
| 1651 | 0.0174 |
| 1652 | 0.0203 |
| 1653 | 0.0335 |
| 1654 | 0.1238 |
| 1655 | 0.2802 |
| 1656 | 0.0364 |
| 1657 | 0.0599 |
| 1658 | 0.0195 |
| 1659 | 0.0091 |
| 1660 | 0.0206 |
| 1661 | 0.0170 |
| 1662 | 0.0274 |
| 1663 | 0.0111 |
| 1664 | 0.0363 |
| 1665 | 0.0170 |
| 1666 | 0.0173 |
| 1667 | 0.0300 |
| 1668 | 0.0487 |
| 1669 | 0.0235 |
| 1670 | 0.0139 |
| 1671 | 0.0229 |
| 1672 | 0.0112 |
| 1673 | 0.0136 |
| 1674 | 0.0169 |
| 1675 | 0.0209 |
| 1676 | 0.0222 |
| 1677 | 0.0211 |
| 1678 | 0.0520 |
| 1679 | 0.0922 |
| 1680 | 0.0717 |
| 1681 | 0.0489 |
| 1682 | 0.0660 |
| 1683 | 0.1036 |
| 1684 | 0.1196 |
| 1685 | 0.1748 |
| 1686 | 0.2150 |
| 1687 | 0.3683 |
| 1688 | 0.6327 |
| 1689 | 0.0270 |
| 1690 | 0.0124 |
| 1691 | 0.0328 |
| 1692 | 0.0107 |
| 1693 | 0.0091 |
| 1694 | 0.0345 |
| 1695 | 0.0344 |
| 1696 | 0.0187 |
| 1697 | 0.0361 |
| 1698 | 0.0323 |
| 1699 | 0.0300 |
| 1700 | 0.0214 |
| 1701 | 0.0245 |
| 1702 | 0.0170 |
| 1703 | 0.0251 |
| 1704 | 0.0358 |
| 1705 | 0.0143 |
| 1706 | 0.0115 |
| 1707 | 0.0186 |
| 1708 | 0.0230 |
| 1709 | 0.0104 |
| 1710 | 0.0339 |
| 1711 | 0.0128 |
| 1712 | 0.0251 |
| 1713 | 0.0237 |
| 1714 | 0.0376 |
| 1715 | 0.0384 |
| 1716 | 0.0249 |
| 1717 | 0.0277 |
| 1718 | 0.0242 |
| 1719 | 0.0210 |
| 1720 | 0.0240 |
| 1721 | 0.0113 |
| 1722 | 0.0275 |
| 1723 | 0.0208 |
| 1724 | 0.0177 |
| 1725 | 0.0247 |
| 1726 | 0.0131 |
| 1727 | 0.0091 |
| 1728 | 0.0292 |
| 1729 | 0.0169 |
| 1730 | 0.0324 |
| 1731 | 0.0130 |
| 1732 | 0.0183 |
| 1733 | 0.0167 |
| 1734 | 0.0108 |
| 1735 | 0.0136 |
| 1736 | 0.0140 |
| 1737 | 0.3554 |
| 1738 | 0.0083 |
| 1739 | 0.0099 |
| 1740 | 0.0061 |
| 1741 | 0.0088 |
| 1742 | 0.0066 |
| 1743 | 0.0060 |
| 1744 | 0.0047 |
| 1745 | 0.0040 |
| 1746 | 0.0088 |
| 1747 | 0.0064 |
| 1748 | 0.0076 |
| 1749 | 0.0042 |
| 1750 | 0.0113 |
| 1751 | 0.0078 |
| 1752 | 0.0067 |
| 1753 | 0.0051 |
| 1754 | 0.0055 |
| 1755 | 0.0084 |
| 1756 | 0.0055 |
| 1757 | 0.0132 |
| 1758 | 0.0075 |
| 1759 | 0.0072 |
| 1760 | 0.0396 |
| 1761 | 0.0139 |
| 1762 | 0.0205 |
| 1763 | 0.0240 |
| 1764 | 0.0331 |
| 1765 | 0.0214 |
| 1766 | 0.0314 |
| 1767 | 0.0167 |
| 1768 | 0.0101 |
| 1769 | 0.0266 |
| 1770 | 0.0689 |
| 1771 | 0.0221 |
| 1772 | 0.0173 |
| 1773 | 0.0473 |
| 1774 | 0.0357 |
| 1775 | 0.0205 |
| 1776 | 0.0377 |
| 1777 | 0.0146 |
| 1778 | 0.0139 |
| 1779 | 0.0166 |
| 1780 | 0.0113 |
| 1781 | 0.0142 |
| 1782 | 0.0236 |
| 1783 | 0.0144 |
| 1784 | 0.0258 |
| 1785 | 0.0114 |
| 1786 | 0.0307 |
| 1787 | 0.0239 |
| 1788 | 0.0245 |
| 1789 | 0.0836 |
| 1790 | 0.0029 |
| 1791 | 0.0176 |
| 1792 | 0.0263 |
| 1793 | 0.0131 |
| 1794 | 0.0126 |
| 1795 | 0.0138 |
| 1796 | 0.0062 |
| 1797 | 0.0233 |
| 1798 | 0.0267 |
| 1799 | 0.0335 |
| 1800 | 0.0173 |
| 1801 | 0.0844 |
| 1802 | 0.0095 |
| 1803 | 0.0247 |
| 1804 | 0.0066 |
| 1805 | 0.0087 |
| 1806 | 0.0036 |
| 1807 | 0.0046 |
| 1808 | 0.0075 |
| 1809 | 0.0013 |
| 1810 | 0.0116 |
| 1811 | 0.0042 |
| 1812 | 0.0032 |
| 1813 | 0.0007 |
| 1814 | 0.0230 |
| 1815 | 0.0222 |
| 1816 | 0.0119 |
| 1817 | 0.0036 |
| 1818 | 0.0023 |
| 1819 | 0.0037 |
| 1820 | 0.0172 |
| 1821 | 0.1906 |
| 1822 | 0.0058 |
| 1823 | 0.0101 |
| 1824 | 0.0093 |
| 1825 | 0.1821 |
| 1826 | 0.0040 |
| 1827 | 0.0094 |
| 1828 | 0.0069 |
| 1829 | 0.0024 |
| 1830 | 0.0036 |
| 1831 | 0.0048 |
| 1832 | 0.0026 |
| 1833 | 0.0015 |
| 1834 | 0.0033 |
| 1835 | 0.0064 |
| 1836 | 0.0072 |
| 1837 | 0.0083 |
| 1838 | 0.0076 |
| 1839 | 0.0023 |
| 1840 | 0.0034 |
| 1841 | 0.0039 |
| 1842 | 0.0012 |
| 1843 | 0.0083 |
| 1844 | 0.0132 |
| 1845 | 0.0073 |
| 1846 | 0.0083 |
| 1847 | 0.0021 |
| 1848 | 0.0083 |
| 1849 | 0.0078 |
| 1850 | 0.0052 |
| 1851 | 0.0052 |
| 1852 | 0.0189 |
| 1853 | 0.0069 |
| 1854 | 0.0151 |
| 1855 | 0.0068 |
| 1856 | 0.0060 |
| 1857 | 0.0074 |
| 1858 | 0.0050 |
| 1859 | 0.0021 |
| 1860 | 0.0039 |
| 1861 | 0.0108 |
| 1862 | 0.0074 |
| 1863 | 0.0043 |
| 1864 | 0.0165 |
| 1865 | 0.0120 |
| 2000 | 0.0394 |
| 2001 | 0.0421 |
| 2002 | 0.0541 |
| 2003 | 0.0350 |
| 2004 | 0.0421 |
| 2005 | 0.0051 |
| 2006 | 0.0148 |
| 2007 | 0.0243 |
| 2008 | 0.0199 |
| 2009 | 0.0058 |
| 2010 | 0.0103 |
| 2011 | 0.0119 |
| 2012 | 0.0158 |
| 2013 | 0.0212 |
| 2014 | 0.0172 |
| 2015 | 0.0105 |
| 2016 | 0.0124 |
| 2017 | 0.0116 |
| 2018 | 0.3345 |
| 2019 | 0.0128 |
| 2020 | 0.0131 |
| 2021 | 0.0151 |
| 2022 | 0.0069 |
| 2023 | 0.0114 |
| 2024 | 0.0136 |
| 2025 | 0.0150 |
| 2026 | 0.1333 |
| 2027 | 0.0103 |
| 2028 | 0.0093 |
| 2029 | 0.0041 |
| 2030 | 0.0147 |
| 2031 | 0.0109 |
| 2032 | 0.0181 |
| 2033 | 0.0184 |
| 2034 | 0.0841 |
| 2035 | 0.0150 |
| 2036 | 0.0100 |
| 2037 | 0.0897 |
| 2038 | 0.0194 |
| 2039 | 0.0129 |
| 2040 | 0.0049 |
| 2041 | 0.0049 |
| 2042 | 0.0065 |
| 2043 | 0.0168 |
| 2044 | 0.0097 |
| 2045 | 0.0105 |
| 2046 | 0.0167 |
| 2047 | 0.0028 |
| 2048 | 0.0402 |
| 2049 | 0.0793 |
| 2050 | 0.1211 |
| 2051 | 0.0485 |
| 2052 | 0.1447 |
| 2053 | 0.0394 |
| 2054 | 0.0124 |
| 2055 | 0.0973 |
| 2056 | 0.0275 |
| 2057 | 0.0036 |
| 2058 | 0.0252 |
| 2059 | 0.3559 |
| 2060 | 0.0193 |
| 2061 | 0.0236 |
| 2062 | 0.0128 |
| 2063 | 0.0162 |
| 2064 | 0.0117 |
| 2065 | 0.0195 |
| 2066 | 0.0061 |
| 2067 | 0.0063 |
| 2068 | 0.0082 |
| 2069 | 0.0072 |
| 2070 | 0.0170 |
| 2071 | 0.1485 |
| 2072 | 0.0195 |
| 2073 | 0.0222 |
| 2074 | 0.0044 |
| 2075 | 0.0041 |
| 2076 | 0.2404 |
| 2077 | 0.0073 |
| 2078 | 0.0049 |
| 2079 | 0.0063 |
| 2080 | 0.0042 |
| 2081 | 0.1111 |
| 2082 | 0.1111 |
| 2083 | 0.9123 |
| 2084 | 0.2095 |
| 2085 | 0.0700 |
| 2086 | 0.0042 |
| 2087 | 0.1430 |
| 2088 | 0.0044 |
| 2089 | 0.0083 |
| 2090 | 0.0078 |
| 2091 | 0.0243 |
| 2092 | 0.0431 |
| 2093 | 0.0120 |
| 2094 | 0.0087 |
| 2095 | 0.0206 |
| 2096 | 0.0045 |
| 2097 | 0.0188 |
| 2098 | 0.0157 |
| 2099 | 0.0126 |
| 2100 | 0.0212 |
| 2101 | 0.0066 |
| 2102 | 0.0456 |
| 2103 | 0.0159 |
| 2104 | 0.0182 |
| 2105 | 0.0068 |
| 2106 | 0.0934 |
| 2107 | 0.0447 |
| 2108 | 0.0199 |
| 2109 | 0.0385 |
| 2110 | 0.0182 |
| 2111 | 0.0330 |
| 2112 | 0.0180 |
| 2113 | 0.0149 |
| 2114 | 0.0689 |
| 2115 | 0.0534 |
| 2116 | 0.0089 |
| 2117 | 0.0187 |
| 2118 | 0.0626 |
| 2119 | 0.0126 |
| 2120 | 0.9640 |
| 2121 | 0.2319 |
| 2122 | 0.0313 |
| 2123 | 0.1769 |
| 2124 | 0.0226 |
| 2125 | 0.0571 |
| 2126 | 0.0079 |
| 2127 | 0.0474 |
| 2128 | 0.0075 |
| 2129 | 0.0482 |
| 2130 | 0.0966 |
| 2131 | 0.0372 |
| 2132 | 0.0652 |
| 2133 | 0.0246 |
| 2134 | 0.3649 |
| 2135 | 0.1308 |
| 2136 | 0.8145 |
| 2137 | 0.0114 |
| 2138 | 0.0086 |
| 2139 | 0.0716 |
| 2140 | 0.4776 |
| 2141 | 0.0261 |
| 2142 | 0.5434 |
| 2143 | 0.1390 |
| 2144 | 0.7852 |
| 2145 | 0.4686 |
| 2146 | 0.0106 |
| 2147 | 0.0120 |
| 2148 | 0.0175 |
| 2149 | 0.0413 |
| 2150 | 0.0075 |
| 2151 | 0.0121 |
| 2152 | 0.0111 |
| 2153 | 0.0101 |
| 2154 | 0.0279 |
| 2155 | 0.0493 |
| 2156 | 0.3217 |
| 2157 | 0.0081 |
| 2158 | 0.0077 |
| 2159 | 0.0104 |
| 2160 | 0.0143 |
| 2161 | 0.0270 |
| 2162 | 0.0077 |
| 2163 | 0.0176 |
| 2164 | 0.0080 |
| 2165 | 0.0241 |
| 2166 | 0.0227 |
| 2167 | 0.0172 |
| 2168 | 0.0196 |
| 2169 | 0.0408 |
| 2170 | 0.0054 |
| 2171 | 0.0191 |
| 2172 | 0.0237 |
| 2173 | 0.0168 |
| 2174 | 0.0177 |
| 2175 | 0.0694 |
| 2176 | 0.1108 |
| 2177 | 0.1283 |
| 2178 | 0.0527 |
| 2179 | 0.0651 |
| 2180 | 0.0281 |
| 2181 | 0.0264 |
| 2182 | 0.0160 |
| 2183 | 0.0194 |
| 2184 | 0.0285 |
| 2185 | 0.0281 |
| 2186 | 0.0452 |
| 2187 | 0.0132 |
| 2188 | 0.0241 |
| 2189 | 0.0322 |
| 2190 | 0.0153 |
| 2191 | 0.0595 |
| 2192 | 0.0184 |
| 2193 | 0.0234 |
| 2194 | 0.0165 |
| 2195 | 0.0228 |
| 2196 | 0.0116 |
| 2197 | 0.0082 |
| 2198 | 0.8031 |
| 2199 | 0.0106 |
| 2200 | 0.0102 |
| 2201 | 0.0225 |
| 2202 | 0.0265 |
| 2203 | 0.0182 |
| 2204 | 0.0244 |
| 2205 | 0.0186 |
| 2206 | 0.0146 |
| 2207 | 0.0344 |
| 2208 | 0.0306 |
| 2209 | 0.0297 |
| 2210 | 0.0251 |
| 2211 | 0.0203 |
| 2212 | 0.0376 |
| 2213 | 0.0592 |
| 2214 | 0.0154 |
| 2215 | 0.0304 |
| 2216 | 0.0193 |
| 2217 | 0.0168 |
| 2218 | 0.0501 |
| 2219 | 0.0134 |
| 2220 | 0.0346 |
| 2221 | 0.0321 |
| 2222 | 0.0250 |
| 2223 | 0.0748 |
| 2224 | 0.0141 |
| 2225 | 0.1821 |
| 2226 | 0.0254 |
| 2227 | 0.0863 |
| 2228 | 0.0161 |
| 2229 | 0.0215 |
| 2230 | 0.0161 |
| 2231 | 0.0108 |
| 2232 | 0.0136 |
| 2233 | 0.0120 |
| 2234 | 0.0708 |
| 2235 | 0.0091 |
| 2236 | 0.0357 |
| 2237 | 0.0228 |
| 2238 | 0.0182 |
| 2239 | 0.0969 |
| 2240 | 0.2589 |
| 2241 | 0.0324 |
| 2242 | 0.0103 |
| 2243 | 0.0162 |
| 2244 | 0.0329 |
| 2245 | 0.0455 |
| 2246 | 0.0132 |
| 2247 | 0.0133 |
| 2248 | 0.0138 |
| 2249 | 0.1338 |
| 2250 | 0.0438 |
| 2251 | 0.1390 |
| 2252 | 0.1320 |
| 2253 | 0.1380 |
| 2254 | 0.1229 |
| 2255 | 0.0118 |
| 2256 | 0.0049 |
| 2257 | 0.0077 |
| 2258 | 0.0149 |
| 2259 | 0.0443 |
| 2260 | 0.0281 |
| 2261 | 0.0160 |
| 2262 | 0.0636 |
| 2263 | 0.0121 |
| 2264 | 0.0119 |
| 2265 | 0.0128 |
| 2266 | 0.0182 |
| 2267 | 0.0448 |
| 2268 | 0.0268 |
| 2269 | 0.0341 |
| 2270 | 0.0245 |
| 2271 | 0.0160 |
| 2272 | 0.0320 |
| 2273 | 0.0266 |
| 2274 | 0.0363 |
| 2275 | 0.0335 |
| 2276 | 0.0266 |
| 2277 | 0.0326 |
| 2278 | 0.0376 |
| 2279 | 0.0156 |
| 2280 | 0.0409 |
| 2281 | 0.0108 |
| 2282 | 0.0072 |
| 2283 | 0.0133 |
| 2284 | 0.0041 |
| 2285 | 0.0121 |
| 2286 | 0.0089 |
| 2287 | 0.0156 |
| 2288 | 0.0146 |
| 2289 | 0.0164 |
| 2290 | 0.0135 |
| 2291 | 0.0106 |
| 2292 | 0.0184 |
| 2293 | 0.0052 |
| 2294 | 0.0094 |
| 2295 | 0.0117 |
| 2296 | 0.0088 |
| 2297 | 0.0104 |
| 2298 | 0.0155 |
| 2299 | 0.0144 |
| 2300 | 0.0134 |
| 2301 | 0.0148 |
| 2302 | 0.0040 |
| 2303 | 0.0053 |
| 2304 | 0.0150 |
| 2305 | 0.0102 |
| 2306 | 0.0071 |
| 2307 | 0.0121 |
| 2308 | 0.0075 |
| 2309 | 0.0059 |
| 2310 | 0.0076 |
| 2311 | 0.0117 |
| 2312 | 0.0079 |
| 2313 | 0.0131 |
| 2314 | 0.0458 |
| 2315 | 0.0331 |
| 2316 | 0.0065 |
| 2317 | 0.5758 |
| 2318 | 0.6775 |
| 2319 | 0.0243 |
| 2320 | 0.0302 |
| 2321 | 0.0193 |
| 2322 | 0.0768 |
| 2323 | 0.1989 |
| 2324 | 0.2467 |
| 2325 | 0.0080 |
| 2326 | 0.0077 |
| 2327 | 0.0085 |
| 2328 | 0.6319 |
| 2329 | 0.9726 |
| 2330 | 0.2492 |
| 2500 | 0.0100 |
| 2501 | 0.0173 |
| 2502 | 0.0508 |
| 2503 | 0.0330 |
| 2504 | 0.0159 |
| 2505 | 0.5423 |
| 2506 | 0.0706 |
| 2507 | 0.0208 |
| 2508 | 0.0432 |
| 2509 | 0.0273 |
| 2510 | 0.0136 |
| 2511 | 0.0051 |
| 2512 | 0.0108 |
| 2513 | 0.0180 |
| 2514 | 0.0054 |
| 2515 | 0.0163 |
| 2516 | 0.0100 |
| 2517 | 0.0034 |
| 2518 | 0.0089 |
| 2519 | 0.0065 |
| 2520 | 0.0105 |
| 2521 | 0.0381 |
| 2522 | 0.0069 |
| 2523 | 0.0096 |
| 2524 | 0.0122 |
| 2525 | 0.0173 |
| 2526 | 0.0548 |
| 2527 | 0.0169 |
| 2528 | 0.0108 |
| 2529 | 0.1385 |
| 2530 | 0.0085 |
| 2531 | 0.0169 |
| 2532 | 0.0421 |
| 2533 | 0.0437 |
| 2534 | 0.0213 |
| 2535 | 0.0380 |
| 2536 | 0.0109 |
| 2537 | 0.0183 |
| 2538 | 0.0097 |
| 2539 | 0.1028 |
| 2540 | 0.0161 |
| 2541 | 0.0584 |
| 2542 | 0.0205 |
| 2543 | 0.2361 |
| 2544 | 0.0806 |
| 2545 | 0.0400 |
| 2546 | 0.0075 |
| 2547 | 0.0114 |
| 2548 | 0.0059 |
| 2549 | 0.0125 |
| 2550 | 0.0049 |
| 2551 | 0.0925 |
| 2552 | 0.0286 |
| 2553 | 0.0246 |
| 2554 | 0.0169 |
| 2555 | 0.0195 |
| 2556 | 0.0062 |
| 2557 | 0.0686 |
| 2558 | 0.0140 |
| 2559 | 0.0248 |
| 2560 | 0.0094 |
| 2561 | 0.0082 |
| 2562 | 0.0065 |
| 2563 | 0.0030 |
| 2564 | 0.0188 |
| 2565 | 0.0033 |
| 2566 | 0.1213 |
| 2567 | 0.0239 |
| 2568 | 0.0671 |
| 2569 | 0.0185 |
| 2570 | 0.0152 |
| 2571 | 0.0261 |
| 2572 | 0.0774 |
| 2573 | 0.1617 |
| 2574 | 0.1316 |
| 2575 | 0.0601 |
| 2576 | 0.0124 |
| 2577 | 0.0131 |
| 2578 | 0.0243 |
| 2579 | 0.1530 |
| 2580 | 0.0450 |
| 2581 | 0.4715 |
| 2582 | 0.0098 |
| 2583 | 0.1222 |
| 2584 | 0.0970 |
| 2585 | 0.0134 |
| 2586 | 0.0100 |
| 2587 | 0.0184 |
| 2588 | 0.0101 |
| 2589 | 0.0343 |
| 2590 | 0.0302 |
| 2591 | 0.0203 |
| 2592 | 0.0153 |
| 2593 | 0.0039 |
| 2594 | 0.0131 |
| 2595 | 0.0099 |
| 2596 | 0.0108 |
| 2597 | 0.0129 |
| 2598 | 0.0140 |
| 2599 | 0.0178 |
| 2600 | 0.0141 |
| 2601 | 0.0126 |
| 2602 | 0.0120 |
| 2603 | 0.0176 |
| 2604 | 0.2338 |
| 2605 | 0.0189 |
| 2606 | 0.0764 |
| 2607 | 0.3887 |
| 2608 | 0.1182 |
| 2609 | 0.0891 |
| 2610 | 0.0783 |
| 2611 | 0.0192 |
| 2612 | 0.0059 |
| 2613 | 0.0110 |
| 2614 | 0.0070 |
| 2615 | 0.0037 |
| 2616 | 0.0038 |
| 2617 | 0.0031 |
| 2618 | 0.0037 |
| 2619 | 0.0501 |
| 2620 | 0.1028 |
| 2621 | 0.1992 |
| 2622 | 0.0027 |
| 2623 | 0.0046 |
| 2624 | 0.0057 |
| 2625 | 0.0057 |
| 2626 | 0.0107 |
| 2627 | 0.0050 |
| 2628 | 0.0206 |
| 2629 | 0.0106 |
| 2630 | 0.0123 |
| 2631 | 0.0085 |
| 2632 | 0.0088 |
| 2633 | 0.0040 |
| 2634 | 0.0047 |
| 2635 | 0.0045 |
| 2636 | 0.0049 |
| 2637 | 0.0050 |
| 2638 | 0.0161 |
| 2639 | 0.0136 |
| 2640 | 0.0350 |
| 2641 | 0.0100 |
| 2642 | 0.0035 |
| 2643 | 0.0073 |
| 2644 | 0.0136 |
| 2645 | 0.0117 |
| 2646 | 0.0083 |
| 2647 | 0.0228 |
| 2648 | 0.0980 |
| 2649 | 0.0072 |
| 2650 | 0.0385 |
| 2651 | 0.0469 |
| 2652 | 0.0296 |
| 2653 | 0.0076 |
| 2654 | 0.0064 |
| 2655 | 0.1097 |
| 2656 | 0.0250 |
| 2657 | 0.0089 |
| 2658 | 0.0259 |
| 2659 | 0.0118 |
| 2660 | 0.0039 |
| 2661 | 0.2012 |
| 2662 | 0.0083 |
| 2663 | 0.0116 |
| 2664 | 0.0152 |
| 2665 | 0.0034 |
| 2666 | 0.1011 |
| 2667 | 0.0114 |
| 2668 | 0.3593 |
| 2669 | 0.0163 |
| 2670 | 0.0064 |
| 2671 | 0.0092 |
| 2672 | 0.4882 |
| 2673 | 0.0058 |
| 2674 | 0.0062 |
| 2675 | 0.7946 |
| 2676 | 0.0085 |
| 2677 | 0.0060 |
| 2678 | 0.0122 |
| 2679 | 0.0233 |
| 2680 | 0.0056 |
| 2681 | 0.0082 |
| 2682 | 0.0296 |
| 2683 | 0.0019 |
| 2684 | 0.0019 |
| 2685 | 0.0060 |
| 2686 | 0.0333 |
| 2687 | 0.0004 |
| 2688 | 0.0413 |
| 2689 | 0.4113 |
| 2690 | 0.4808 |
| 2691 | 0.0068 |
| 2692 | 0.0047 |
| 2693 | 0.0424 |
| 2694 | 0.0165 |
| 2695 | 0.0318 |
| 2696 | 0.0171 |
| 2697 | 0.0262 |
| 2698 | 0.1294 |
| 2699 | 0.2395 |
| 2700 | 0.0623 |
| 2701 | 0.1634 |
| 2702 | 0.0505 |
| 2703 | 0.4118 |
| 2704 | 0.3157 |
| 2705 | 0.0189 |
| 2706 | 0.0024 |
| 2707 | 0.0050 |
| 2708 | 0.0006 |
| 2709 | 0.0023 |
| 2710 | 0.0012 |
| 2711 | NT |
| 2712 | 0.0028 |
| 2713 | 0.0007 |
| 2714 | 0.0003 |
| 2715 | 0.0014 |
| 2716 | 0.0014 |
| 2717 | 0.0356 |
| 2718 | 0.3303 |
| 2719 | 0.0439 |
| 2720 | 0.1014 |
| 2721 | 0.1902 |
| 2722 | 0.1048 |
| 2723 | 0.2001 |
| 2724 | 0.0353 |
| 2725 | 0.0481 |
| 2726 | 0.0655 |
| 2727 | 0.3483 |
| 2728 | 0.9644 |
| 2729 | 0.0020 |
| 2730 | 0.0940 |
| 2731 | 0.0015 |
| 2732 | 0.0017 |
| 2733 | 0.0025 |
| 2734 | 0.0057 |
| 2735 | 0.0022 |
| 2736 | 0.0011 |
| 2737 | 0.0054 |
| 2738 | 0.0023 |
| 2739 | 0.0032 |
| 2740 | 0.0048 |
| 2741 | 0.0023 |
| 2742 | 0.0046 |
| 2743 | 0.0022 |
| 2744 | 0.0034 |
| 2743 | 0.0022 |
| 2744 | 0.0034 |
| 2745 | 0.0023 |
| 2746 | 0.0037 |
| 2747 | 0.0028 |
| 2748 | 0.0252 |
| 2749 | 0.0019 |
| 2750 | 0.1999 |

It is to be appreciated that the Detailed Description section, and not the Summary and Abstract sections, is intended to be used to interpret the claims. The Summary and Abstract sections can set forth one or more but not all exemplary embodiments of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. A compound of Formula (I): 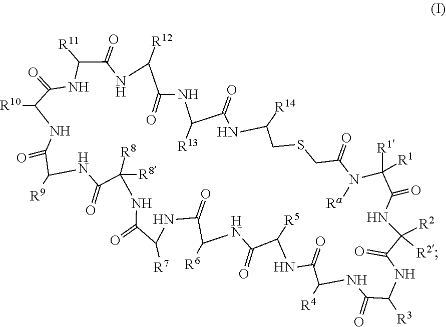
or a pharmaceutically acceptable salt thereof, wherein:
R¹ is selected from C₁-C₆alkoxyC₁-C₆alkyl; C₁-C₆alkyl; C₁-C₆alkylaminoC₁-C₆alkyl; C₁-C₆alkylcarbonylaminoC₁-C₆alkyl; aminoC₁-C₆alkyl; aminocarbonylC₁-C₆alkyl; arylC₁-C₆alkyl; arylcarbonylaminoC₁-C₆alkyl; carboxyC₁-C₆alkyl; cyanoC₁-C₆alkyl; heteroarylC₁-C₆alkyl; heterocyclylC₁-C₆alkyl; hydroxyC₁-C₆alkyl; NH₂C(X)NHC₁-C₆alkyl, wherein X is O or NH; and H₂NC(X)N [symbol] C—, where N [symbol] C represents an azetidine, piperidine, or pyrrolidine ring; wherein the aryl part of the arylC₁-C₆alkyl and the arylcarbonylaminoC₁-C₆alkyl and the heteroaryl part of the heteroarylC₁-C₆alkyl are optionally substituted with one, two, three, four, or five groups independently selected from C₁-C₆alkoxy, C₁-C₆alkylcarbonylamino, C₂-C₆alkynyloxy, aminocarbonyl, aryl optionally substituted with one or two carboxy groups, arylC₁-C₆alkoxy, carboxy, carboxyC₁-C₆alkoxy, halo, and trifluoromethyl;
R^{1′} is hydrogen or C₁-C₆alkyl;
R² is selected from C₁-C₆alkoxyC₁-C₆alkyl; arylC₁-C₆alkyl; azidoC₁-C₆alkyl; biscarboxyCHC₁-C₆alkyl; carboxyC₁-C₆alkyl; and heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, C₁-C₆alkylcarbonylamino, C₂-C₆alkynyloxy, amino, aminoC₁-C₆alkyl, aminocarbonyl, aryl optionally substituted with one or two carboxy groups, arylC₁-C₆alkoxy, arylcarbonyl, azido, carboxy, carboxyC₁-C₆alkoxy, carboxyC₁-C₆alkyl, cyano, halo, haloC₁-C₆alkoxy, hydroxy, nitro, and trifluoromethyl;
R^{2′} is hydrogen or C₁-C₆alkyl;
R³ is selected from C₁-C₆alkoxyC₁-C₆alkyl; aminocarbonylC₁-C₆alkyl, arylC₁-C₆alkoxyC₁-C₆alkyl, arylC₁-C₃alkyl, carboxyC₁-C₆alkyl, furylC₁-C₃alkyl, hydroxyC₁-C₆alkyl, HOS(O)₂C₁-C₃alkyl, CH₃S(O)₂NHC(O)(C₁-C₃alkyl), and tetrazolylC₁-C₃alkyl; wherein the aryl part of the arylC₁-C₃alkyl is optionally substituted with one, two, or three, aminoC₁-C₃alkyl groups;
R⁴ is selected from arylC₁-C₆alkyl and heteroarylC₁-C₆alkyl, wherein the aryl part of the arylC₁-C₆alkyl and the heteroaryl part of the heteroarylC₁-C₆alkyl are optionally substituted with one or more groups independently selected from C₁-C₆alkoxy, C₁-C₆alkyl, amino, cyano, C₁-C₆fluoroalkyl, halo, and hydroxy;
R⁵ is selected from C₁-C₆alkoxyC₁-C₆alkyl; C₁-C₆alkyl; aryl; arylC₁-C₆alkyl; cyanoC₁-C₆alkyl; C₃-C₈cycloalkyl; (C₃-C₈cycloalkyl)C₁-C₆alkyl; fluoroC₁-C₆alkyl; heteroarylC₁-C₆alkyl; and hydroxyC₁-C₆alkyl; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, amino, aminoC₁-C₆alkyl, aminocarbonyl, aryl, arylC₁-C₆alkoxy, aryloxy, carboxyC₁-C₆alkoxy, cyano, (C₃-C₆cycloalkyl)oxy, carboxy, halo, heteroaryl, and hydroxy, wherein the aryl is further optionally substituted with one, two, or three groups independently selected from C₁-C₃alkyl, C₁-C₃alkylcarbonylamino, carboxy, and hydroxy;
R⁶ is selected from aryl-arylC₁-C₃alkyl, aryl-heteroarylC₁-C₃alkyl, heteroaryl-arylC₁-C₃alkyl, and heteroaryl-heteroarylC₁-C₃alkyl, wherein each aryl and each heteroaryl are optionally substituted with one or more groups independently selected from C₁-C₆alkoxy, C₁-C₆alkyl, amino, cyano, C₁-C₆fluoroalkyl, halo, and hydroxyl;
R⁷ is selected from hydrogen; C₁-C₆alkyl; C₁-C₆alkylcarbonylaminoC₁-C₆alkyl; C₂-C₆alkynyl; aminoC₁-C₆alkyl; aminocarbonylC₁-C₆alkyl; aryl; arylC₁-C₆alkyl; carboxyC₁-C₆alkyl; C₃-C₈cycloalkyl; (C₃-C₈cycloalkyl)C₁-C₆alkyl; haloarylcarbonylaminoC₁-C₆alkyl; heteroarylC₁-C₆alkyl; hydroxyC₁-C₆alkyl; and NH₂C(X)NHC₁-C₆alkyl, where X is O or NH; wherein the aryl part of the arylC₁-C₆alkyl and the heteroaryl part of the heteroarylC₁-C₆alkyl are optionally substituted with one, two, three, four, or five groups independently selected from C₂-C₆alkynyloxy, arylC₁-C₆alkoxy, carboxy, carboxyC₁-C₆alkoxy, haloC₁-C₆alkoxy, and hydroxy;
R⁸ is selected from C₁-C₆alkyl; C₁-C₆alkylcarbonylaminoC₁-C₆alkyl; aminoC₁-C₆alkyl; (C₇H₁₅O₆)aminoC₁-C₆alkyl; aminocarbonylC₁-C₆alkyl; arylC₁-C₆alkyl; carboxyC₁-C₆alkyl; heterocyclyl; heteroarylC₁-C₆alkyl; and hydroxyC₁-C₆alkyl; wherein the aryl part of the arylC₁-C₆alkyl and the arylcarbonylaminoC₁-C₆alkyl are optionally substituted with one, two, three, four, or five groups independently selected from aminoC₁-C₆alkyl, halo, and hydroxy;
R^{8′} is hydrogen or R⁸ and R^{8′}, together with the atoms to which they are attached, form a C₃-C₈cycloalkyl ring;
R⁹ is selected from C₁-C₆alkyl; arylC₁-C₆alkyl; and C₃-C₈cycloalkylC₁-C₆alkyl; wherein the aryl part of the arylC₁-C₆alkyl is optionally substituted with one, two, three, four, or five groups independently selected from halo and hydroxy;
R¹⁰ is selected from C₁-C₆alkyl; C₂-C₆alkynyl; aminoC₁-C₆alkyl; aminocarbonylC₁-C₆alkyl; arylC₁-C₆alkyl; carboxyC₁-C₆alkyl; hydroxyC₁-C₆alkyl; (C₇H₁₅O₆)aminoC₁-C₆alkyl; C₁-C₆alkylcarbonylaminoC₁-C₆alkyl; heteroarylC₁-C₆alkyl; and NH₂C(X)NHC₁-C₆alkyl, wherein X is O or NH; wherein the aryl part of the arylC₁-C₆alkyl is optionally substituted with one, two, three, four, or five groups independently selected from C₂-C₆alkynyloxy, and arylC₁-C₆alkoxy;
R¹¹ is selected from C₁-C₈alkyl; arylC₁-C₆alkyl; C₃-C₈cycloalkylC₁-C₆alkyl; and heteroarylC₁-C₆alkyl; wherein the aryl part of the arylC₁-C₆alkyl is optionally substituted with one, two, three, four, or five groups independently selected from C₁-C₆alkoxy, C₁-C₆alkyl, amino, aminoC₁-C₆alkoxy, aminoC₁-C₆alkyl cyano, halo, hydroxy, and trifluoromethyl;
R¹² is selected from C₁-C₆alkyl, C₂-C₆alkynyl, arylC₁-C₆alkyl, carboxyC₁-C₆alkyl, and hydroxyC₁-C₆alkyl; wherein the aryl part of the arylC₁-C₆alkyl is optionally substituted with one, two, three, four, or five groups independently selected from C₂-C₆alkynyloxy and arylC₁-C₆alkoxy;
R¹³ is selected from C₁-C₆alkyl, C₁-C₆alkylcarbonylaminoC₁-C₆alkyl, aminoC₁-C₆alkyl, aminocarbonylC₁-C₆alkyl, arylC₁-C₆alkyl, carboxyC₁-C₆alkyl, haloarylcarbonylaminoC₁-C₆alkyl, heteroarylC₁-C₆alkyl, hydroxyC₁-C₆alkyl, and NH₂C(X)NHC₁-C₆alkyl, wherein X is 0 or NH; wherein the aryl part of the arylC₁-C₆alkyl is optionally substituted with one, two, three, four, or five groups independently selected from C₂-C₆alkynyloxy and arylC₁-C₆alkoxy;
R¹⁴ is aminocarbonyl; carboxy; or —C(O)NR^{14′}CR¹⁵R^{15′}R^{15″}, wherein
R^{14′} is hydrogen or C₁-C₆alkyl, or R¹⁵ and R^{14′}, together with the atoms to which they are attached, form an azetidine, morpholine, piperazine, piperidine, or pyrrolidine ring, wherein each ring is optionally substituted with an amino or a hydroxy group;
R¹⁵ is selected from hydrogen, C₁-C₆alkyl, C₁-C₆alkylcarbonylaminoC₁-C₆alkyl, C₂-C₆alkynyl, aminoC₁-C₆alkyl, aminocarbonylC₁-C₆alkyl, arylC₁-C₆alkyl, azidoC₁-C₆alkyl, carboxy, carboxyC₁-C₆alkyl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl is optionally substituted with one, two, three, four, or five groups independently selected from arylC₁-C₆alkoxy and hydroxy;
R^{15′} is hydrogen or C₁-C₆alkyl; or R¹⁵ and R^{15′}, together with the atoms to which they are attached, form a C₃-C₈cycloalkyl ring; and
R^{15″} is hydrogen; amincarbonyl, carboxy, or —(CH₂)ₙC(O)NHCHR¹⁶R^{16′}; wherein
n is 0, 1, or 2;
R¹⁶ is selected from hydrogen, C₁-C₆alkoxyC₁-C₆alkyl, C₁-C₆alkylC₂-C₆alkynyl, C₂-C₆alkynyl, aminoC₁-C₆alkyl, arylC₁-C₆alkyl, carboxy, carboxyC₁-C₆alkyl, heteroaryl, heteroarylC₁-C₆alkyl, hydroxyC₁-C₆alkyl, and NH₂C(X)NHC₁-C₆alkyl, wherein X is O or NH; wherein the aryl part of the arylC₁-C₆alkyl and the heteroaryl are optionally substituted with one, two, three, four, or five groups independently selected from C₁-C₆alkyl, arylC₁-C₆alkoxy, and hydroxy; and
R^{16′} is hydrogen, C₁-C₆alkyl, aminocarbonyl, carboxy, or —(CH₂)ₘC(O)NHCHR¹⁷R^{17′}; wherein
m is 0, 1, or 2;
R¹⁷ is C₂-C₆alkynyl; and
R^{17′} is aminocarbonyl or carboxy; and
R^{a} is hydrogen or C₁-C₆alkyl; or R¹ and R^{a}, together with the atoms to which they are attached, form an azetidine, morpholine, piperidine, piperazine, or pyrrolidine ring, wherein each ring is optionally substituted with an amino or a hydroxy group.

2. The compound of claim 1, or the pharmaceutically acceptable salt thereof, wherein R¹ is selected from C₁-C₆alkyl, aminoC₁-C₆alkyl, aminocarbonylC₁-C₆alkyl, arylC₁-C₆alkyl, heteroarylC₁-C₆alkyl, heterocyclylC₁-C₆alkyl, and hydroxyC₁-C₆alkyl, wherein the aryl part of the arylC₁-C₆alkyl is optionally substituted with one, two, or three groups independently selected from halo and carboxyC₁-C₆alkoxy; and
R^{1′} is hydrogen.

3. The compound of claim 1, or the pharmaceutically acceptable salt thereof, wherein R² is selected from arylC₁-C₆alkyl and heteroarylC₁-C₆alkyl, wherein the aryl part of the arylC₁-C₆alkyl is optionally substituted with one, two, or three groups independently selected from carboxy, carboxyC₁-C₆alkoxy, cyano, halo, hydroxy, and nitro; and
R^{2′} is hydrogen.

4. The compound of claim 1, or the pharmaceutically acceptable salt thereof, wherein R³ is aminocarbonylC₁-C₆alkyl or carboxyC₁-C₆alkyl.

5. The compound of claim 1, or the pharmaceutically acceptable salt thereof, wherein R⁴ is arylC₁-C₆alkyl; wherein the aryl part of the arylC₁-C₆alkyl is optionally substituted with one, two, or three groups independently selected from C₁-C₆alkyl, halo, and trifluoromethyl.

6. The compound of claim 1, or the pharmaceutically acceptable salt thereof, wherein R⁵ is C₁-C₆alkyl or arylC₁-C₆alkyl; wherein the aryl part of the arylC₁-C₆alkyl is optionally substituted with one, two, or three groups independently selected from carboxy, carboxyC₁-C₆alkoxy, and hydroxy.

7. The compound of claim 1, or the pharmaceutically acceptable salt thereof, wherein R⁶ is biphenylC₁-C₆alkyl.

8. The compound of claim 1, or the pharmaceutically acceptable salt thereof, wherein R⁷ is selected from C₁-C₆alkyl, arylC₁-C₆alkyl, carboxyC₁-C₆alkyl, and NH₂C(X)NHC₁-C₆alkyl, wherein X is O or NH; wherein the aryl part of the arylC₁-C₆alkyl is optionally substituted with one, two, or three groups independently selected from carboxy, carboxyC₁-C₆alkoxy and hydroxy.

9. The compound of claim 1, or the pharmaceutically acceptable salt thereof, wherein R⁸ is C₁-C₆alkyl or aminoC₁-C₆alkyl; and R^{8′} is hydrogen.

10. The compound of claim 1, or the pharmaceutically acceptable salt thereof, wherein R⁹ is C₁-C₆alkyl.

11. The compound of claim 1, or the pharmaceutically acceptable salt thereof, wherein R¹⁰ is aminoC₁-C₆alkyl or aminocarbonylC₁-C₆alkyl.

12. The compound of claim 1, or the pharmaceutically acceptable salt thereof, wherein R¹¹ is C₁-C₆alkyl or C₃-C₆cycloalkylC₁-C₃alkyl.

13. The compound of claim 1, or the pharmaceutically acceptable salt thereof, wherein R¹² is C₁-C₄alkyl or hydroxyC₁-C₄alkyl.

14. The compound of claim 1, or the pharmaceutically acceptable salt thereof, wherein R¹³ is aminoC₁-C₆alkyl, aminocarbonylC₁-C₂alkyl, carboxyC₁-C₆alkyl, or hydroxyC₁-C₄alkyl.

15. The compound of claim 1, or the pharmaceutically acceptable salt thereof, wherein R¹⁴ is aminocarbonyl or —C(O)NHCHR¹⁵C(O)NH₂, and wherein R¹⁵ is hydrogen, C₁-C₆alkyl, aminoC₁-C₆alkyl.

16. The compound of claim 1, or the pharmaceutically acceptable salt thereof, wherein R¹⁵ is hydrogen or C₁-C₆alkyl.

17. The compound of claim 1, or the pharmaceutically acceptable salt thereof, wherein R¹⁶ is hydrogen or C₂-C₄alkynyl.

18. The compound of claim 1, or the pharmaceutically acceptable salt thereof, wherein R^{a} is methyl.

19. The compound of claim 1, or the pharmaceutically acceptable salt thereof, wherein one, two, or all of R^{1′}, R^{2′}, and R^{8′} are methyl.

20. The compound of claim 1, or the pharmaceutically acceptable salt thereof, wherein
R¹ is selected from C₁-C₄alkyl, C₁-C₄alkylcarbonylaminoC₂-C₄alkyl, aminoC₁-C₃alkyl, aminocarbonylC₁-C₂alkyl, arylC₁-C₂alkyl, arylcarbonylaminoC₁-C₂alkyl, carboxypropyl, cyanomethyl; heteroarylmethyl, heterocyclylmethyl, hydroxyC₂-C₃alkyl, methoxyC₁-C₂alkyl, methylaminoC₁-C₂alkyl, NH₂C(X)NHpropyl, wherein X is O or NH, and H₂NC(X)piperidinyl, wherein the aryl part of the arylC₁-C₂alkyl and the arylcarbonylaminoC₁-C₆alkyl is optionally substituted with one, two, or three groups independently selected from aminocarbonyl, arylmethoxy, carboxy, carboxymethoxy, carboxyphenyl, halo, methoxy, methylcarbonylamino, propynyloxy, and trifluoromethyl;
R^{1′} is hydrogen or methyl;
R² is selected from arylC₁-C₂alkyl, azidoC₁-C₂alkyl, biscarboxyethyl, carboxyC₁-C₃alkyl, methoxyC₁-C₂alkyl, and heteroarylC₁-C₂alkyl; wherein the aryl part of the arylC₁-C₂alkyl and the heteroaryl part of the heteroarylC₁-C₂alkyl are optionally substituted with one, two, three, four, or five groups independently selected from C₁-C₄alkyl, amino, aminocarbonyl, aminomethyl, arylcarbonyl, arylmethoxy, azido, carboxy, carboxymethoxy, carboxymethyl, carboxyphenyl, cyano, halo, hydroxy, methoxy, methylcarbonylamino, nitro, propynyloxy, trifluoromethoxy, and trifluoromethyl;
R^{2′} is hydrogen or methyl;
R³ is selected from aminocarbonylmethyl, arylC₁-C₃alkyl, arylmethoxymethyl, carboxyC₁-C₂alkyl, furylC₁-C₃alkyl, hydroxyC₁-C₂alkyl, methoxymethyl, and tetrazolylmethyl, HOS(O)₂C₁-C₃alkyl, and CH₃S(O)₂NHC(O)(C₁-C₃alkyl); wherein the aryl part of the arylC₁-C₃alkyl is optionally substituted with one, two, or three aminoC₁-C₃alkyl groups;
R⁴ is selected from arylC₁-C₂alkyl and heteroarylmethyl; wherein the aryl part of the arylC₁-C₂alkyl and the heteroaryl part of the heteroarylmethyl are optionally substituted with one, two, three, four, or five groups independently selected from amino, cyano, halo, hydroxy, methoxy, methyl, and trifluoromethyl;
R⁵ is selected from C₁-C₅alkyl, arylmethyl, cyanomethyl, C₃-C₆cycloalkyl, (C₃-C₆cycloalkyl)methyl, heteroarylmethyl, hydroxyC₁-C₂alkyl, methoxymethyl, and phenyl; wherein the aryl part of the arylmethyl is optionally substituted with one, two, three, four, or five groups independently selected from amino, aminomethyl, aminocarbonyl, aryl, arylmethoxy, aryloxy, carboxymethoxy, carboxy, cyano, (C₃-C₆cycloalkyl)oxy, halo, heteroaryl, hydroxy, and propynyloxy; wherein the aryl is further optionally substituted with one, two, or three groups independently selected from C₁-C₃alkyl, C₁-C₃alkylcarbonylamino, carboxy, and hydroxy;
R⁶ is biphenylmethyl;
R⁷ is selected from hydrogen, C₁-C₅alkyl, aminoC₁-C₄alkyl, aminocarbonylethyl, aminocarbonylmethyl, arylmethyl, butynyl, C₃-C₆cycloalkyl, (C₃-C₆cycloalkyl)methyl, carboxyethyl, haloarylcarbonylaminopropyl, heteroarylpropyl, hydroxyC₂alkyl, methylcarbonylaminoC₂-C₄alkyl, phenyl, and NH₂C(X)NHC₂-C₄alkyl, wherein X is O or NH; wherein the aryl part of the arylmethyl is optionally substituted with one, two, or three groups independently selected from arylmethoxy, carboxy, carboxymethoxy, hydroxy, propynyloxy, and trifluoromethoxy;
R⁸ is selected from C₁-C₄alkyl, C₁-C₄alkylcarbonylaminoC₁-C₄alkyl, aminoC₁-C₄alkyl, aminocarbonylC₁-C₂alkyl, (C₇H₁₅O₆)aminomethyl, arylmethyl, carboxyC₁-C₃alkyl, haloarylcarbonylaminopropyl, heterocyclyl, or heteroarylmethyl, and hydroxymethyl; wherein the aryl part of the arylmethyl is optionally substituted with one, two, or three groups independently selected from hydroxy and aminomethyl;
R^{8′} is hydrogen or R⁸ and R^{8′}, together with the atoms to which they are attached, form a cyclopropyl ring;
R⁹ is selected from C₁-C₄alkyl, arylmethyl, and cyclohexylmethyl; wherein the aryl part of the arylmethyl is optionally substituted with one, two, or three hydroxy groups;
R¹⁰ is selected from C₁-C₄alkyl, aminoC₁-C₄alkyl, aminocarbonylC₁-C₂alkyl, (C₇H₁₅O₆)aminomethyl, arylmethyl, butylcarbonylaminoethyl, butynyl, carboxyC₁-C₃alkyl, heteroarylmethyl, hydroxyC₁-C₂alkyl, NH₂C(NH)NHmethyl; wherein the aryl part of the arylmethyl is optionally substituted with one, two, or three groups independently selected from arylmethoxy and propynyloxy;
R¹¹ is selected from C₄-C₈alkyl, arylC₁-C₂alkyl, C₃-C₆cycloalkylC₁-C₂alkyl, and heteroarylmethyl; wherein the aryl part of the arylC₁-C₂alkyl is optionally substituted with one, two, or three groups independently selected from amino, aminoethoxy, aminomethyl, cyano, halo, hydroxy, methoxy, methyl, and trifluoromethyl;
R¹² is selected from C₃-C₄alkyl, arylmethyl, carboxybutyl, hydroxyC₁-C₃alkyl, and propynyl; wherein the aryl part of the arylmethyl is optionally substituted with one, two, or three groups independently selected from arylmethoxy and propynyloxy;
R¹³ is selected from C₃-C₄alkyl, C₁-C₄alkylcarbonylaminoC₂-C₄alkyl, aminoC₁-C₄alkyl, aminocarbonylC₁-C₂alkyl, arylmethyl, carboxyC₁-C₃alkyl, haloarylcarbonylaminopropyl, heteroarylmethyl, hydroxyC₁-C₃alkyl, and NH₂C(X)NHpropyl, wherein X is O or NH; wherein the aryl part of the arylmethyl is optionally substituted with one, two, or three groups independently selected from arylmethoxy and propyynyloxy;
R¹⁴ is aminocarbonyl; carboxy; or —C(O)NR^{14′}CR¹⁵R^{15′}R^{15″}; wherein
R^{14′} is hydrogen or methyl; or R¹⁵ and R^{14′}, together with the atoms to which they are attached, form a pyrrolidine ring;
R¹⁵ is selected from hydrogen, C₁-C₂alkyl, C₁-C₄alkylcarbonylaminoC₁-C₃alkyl, aminoC₁-C₄alkyl, aminocarbonylC₁-C₂alkyl, arylmethyl, azidoC₂-C₄alkyl, carboxy, carboxyC₁-C₃alkyl, heteroarylmethyl, hydroxymethyl, NH₂C(NH)NHpropyl, and propynyl; and wherein the aryl part of the arylmethyl is optionally substituted with one, two, or three groups independently selected from arylmethoxy and hydroxy;
R^{15′} is hydrogen or methyl; or R¹⁵ and R^{15′}, together with the atoms to which they are attached, form a cyclopropyl ring; and
R^{15″} is hydrogen, aminocarbonyl, or carboxy; or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxy, carboxyethyl, heteroaryl, heteroarylethyl, hydroxymethyl, methoxymethyl, NH₂C(NH)NHpropyl, and propynyl; wherein the aryl part of the arylC₁-C₆alkyl and the heteroaryl are optionally substituted with one, two, three, four, or five groups independently selected from arylmethoxy, hydroxy, and methyl; and
R^{16′} is hydrogen, aminocarbonyl, carboxy, methyl, or —(CH₂)ₘC(O)NHCHR¹⁷R^{17′}; wherein
m is 0, 1, or 2;
R¹⁷ is propynyl; and
R^{17′} is aminocarbonyl or carboxy; and
R^{a} is hydrogen or methyl; or R¹ and R^{a}, together with the atoms to which they are attached, form a piperazine or pyrrolidine ring, wherein each ring is optionally substituted with an amino or a hydroxy group.

21. The compound of claim 20, or the pharmaceutically acceptable salt thereof, wherein R¹ is selected from C₁-C₆alkyl, aminoC₁-C₃alkyl, aminocarbonylC₁-C₂alkyl, arylC₁-C₂alkyl, heteroarylmethyl, heterocyclulC₁-C₆alkyl, and hydroxyC₂-C₃alkyl, and wherein the aryl part of the arylC₁-C₂alkyl is optionally substituted with one, two, or three groups independently selected from carboxymethoxy and halo; and
R^{1′} is hydrogen.

22. The compound of claim 20 or claim 21, or the pharmaceutically acceptable salt thereof, wherein R^{a} is hydrogen.

23. The compound of claim 20, or the pharmaceutically acceptable salt thereof, wherein
R¹ is selected from C₁-C₄alkyl, C₁-C₄alkylcarbonylaminoC₂-C₄alkyl, aminoC₁-C₃alkyl, aminocarbonylC₁-C₂alkyl, arylC₁-C₂alkyl, arylcarbonylaminoC₁-C₂alkyl, carboxypropyl, cyanomethyl, heteroarylmethyl, heterocyclmethyl, hydroxyC₂-C₃alkyl, methoxyC₁-C₂alkyl, methylaminoC₁-C₂alkyl, NH₂C(NH)NHpropyl, and H₂NC(NH)piperidinyl; wherein the aryl part of the arylC₁-C₂alkyl and the arylcarbonylaminoC₁-C₂alkyl is optionally substituted with one, two, or three groups independently selected from aminocarbonyl, carboxy, carboxymethoxy, halo, methoxy, methylcarbonylamino, propynyloxy, and trifluoromethyl;
R^{1′} is hydrogen;
R² is selected from arylC₁-C₂alkyl, azidoC₁-C₂alkyl, carboxypropyl, heteroarylC₁-C₂alkyl, and methoxyC₁-C₂alkyl; wherein the aryl part of the arylC₁-C₂alkyl and the heteroaryl part of the heteroarylC₁-C₂alkyl are optionally substituted with one or more groups independently selected from C₁-C₄alkyl, amino, aminocarbonyl, aminomethyl, arylcarbonyl, arylmethoxy, carboxy, carboxymethoxy, carboxymethyl, cyano, halo, hydroxy, methoxy, methylcarbonylamino, nitro, propynyloxy, trifluoromethoxy, and trifluoromethyl;
R^{2′} is hydrogen or methyl;
R³ is selected from aminocarbonylmethyl; arylC₁-C₃alkyl, carboxymethyl, furylC₁-C₃alkyl, hydroxyC₁-C₃alkyl, HOS(O)₂C₁-C₃alkyl, CH₃S(O)₂NHC(O)(C₁-C₃alkyl), and tetrazolyl;
wherein the aryl part of the arylC₁-C₃alkyl is optionally substituted with one, two, or three aminoC₁-C₃alkyl groups;
R⁴ is selected from arylC₁-C₂alkyl and heteroarylmethyl, and wherein the aryl part of the arylC₁-C₂alkyl and the heteroaryl part of the heteroarylmethyl are optionally substituted with one or more groups independently selected from amino, halo, hydroxy, methoxy, methyl, and trifluoromethyl;
R⁵ is selected from C₁-C₅alkyl, arylmethyl, C₃-C₆cycloalkyl, (C₃-C₆cycloalkyl)methyl, heteroarylmethyl, hydroxyC₂alkyl, methoxymethyl, and phenyl; wherein the aryl part of the arylmethyl is optionally substituted with one, two, three, four, or five groups independently selected from amino, aminocarbonyl, aminomethyl, aryl, arylmethoxy, aryloxy, carboxy, carboxymethoxy, (C₃-C₆cycloalkyl)oxy, cyano, halo, heteroaryl, hydroxy, and propynyloxy; wherein the aryl is further optionally substituted with C₁-C₃alkyl, C₁-C₃alkylcarbonylamino, carboxy, and hydroxy;
R⁶ is biphenylmethyl;
R⁷ is selected from C₁-C₅alkyl, aminoC₁-C₄alkyl, aminocarbonylethyl, aminocarbonylmethyl, arylmethyl, butynyl, carboxyethyl, C₃-C₆cycloalkyl, (C₃-C₆cycloalkyl)methyl, heteroarylmethyl, hydroxyC₂alkyl, methylcarbonylaminoC₂-C₄alkyl, phenyl, and NH₂C(X)NHC₂-C₄alkyl, wherein X is O or NH; wherein the aryl part of the arylmethyl is optionally substituted with one, two, or three groups independently selected from arylmethoxy, carboxy, carboxymethoxy, hydroxy, propynyloxy, and trifluoromethoxyl;
R⁸ is selected from C₁-C₄alkyl, C₁-C₄alkylcarbonylaminoC₁-C₄alkyl, aminoC₁-C₄alkyl, aminocarbonylC₁-C₂alkyl, (C₇H₁₅O₆)aminomethyl, arylmethyl, carboxyC₁-C₃alkyl, heteroarylmethyl, and hydroxymethyl; wherein the aryl part of the arylmethyl is optionally substituted with one, two, or three groups independently selected from aminomethyl and hydroxy;
R^{8′} is hydrogen;
R⁹ is selected from C₁-C₄alkyl, cyclohexylmethyl, and phenylmethyl; wherein the phenyl part of the phenylmethyl is optionally substituted with one or two hydroxy groups;
R¹⁰ is selected from aminoC₁-C₄alkyl, aminocarbonylC₁-C₂alkyl, (C₇H₁₅O₆)aminomethyl, butynyl, butylcarbonylaminoethyl; carboxyC₁-C₃alkyl, heteroarylmethyl, hydroxyC₁-C₂alkyl, and NH₂C(NH)NHmethyl;
R¹¹ is selected from C₄-C₈alkyl, arylC₁-C₂alkyl, C₃-C₆cycloalkylC₁-C₂alkyl, and heteroarylmethyl; wherein the aryl part of the arylC₁-C₂alkyl is optionally substituted with one, two, or three groups independently selected from aminomethyl, chloro, fluoro, hydroxy, methoxy, methyl, and trifluoromethyl;
R¹² is selected from C₃-C₄alkyl, carboxybutyl, hydroxyC₁-C₃alkyl, phenylmethyl, and propynyl; wherein the phenyl part of the phenylmethyl is optionally substituted with one, two, or three propynyloxy;
R¹³ is selected from C₃-C₄alkyl, C₁-C₄alkylcarbonylaminoC₂-C₄alkyl, aminoC₁-C₄alkyl, aminocarbonylC₁-C₂alkyl, arylmethyl, carboxyC₁-C₃alkyl, haloarylcarbonylaminopropyl; hydroxyC₁-C₃alkyl, heteroarylmethyl, and NH₂C(X)NHpropyl, wherein X is O or NH; wherein the aryl part of the arylmethyl is optionally substituted with one, two, or three groups independently selected from arylmethoxy and propynyloxy;
R¹⁴ is aminocarbonyl or —C(O)NR^{14′}CR¹⁵R^{15′}R^{15″}, wherein
R^{14′} is hydrogen or methyl;
R¹⁵ is selected from hydrogen; C₁-C₂alkyl, C₁-C₄alkylcarbonylaminoC₁-C₃alkyl, aminoC₁-C₄alkyl, aminocarbonylC₁-C₂alkyl, arylmethyl, azidoC₂-C₃alkyl, carboxy; carboxyC₁-C₂alkyl, heteroarylmethyl, hydroxymethyl, propynyl, and NH₂C(NH)NHpropyl; wherein the aryl part of the arylmethyl is optionally substituted with one, two, or three groups independently selected from arylmethoxy and hydroxy;
R^{15′} is hydrogen or methyl; or R¹⁵ and R^{15′}, together with the atoms to which they are attached, form a cyclopropyl ring; and
R^{15″} is hydrogen, aminocarbonyl, carboxy, or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxy, carboxyethyl, heteroaryl, heteroarylethyl, hydroxymethyl, methoxymethyl, NH₂C(NH)NHpropyl, and propynyl; wherein the aryl part of the arylmethyl and the heteroaryl are optionally substituted with one or more groups independently selected from arylmethoxy and methyl; and
R^{16′} is hydrogen, aminocarbonyl, carboxy, methyl, or —(CH₂)ₘC(O)NHCHR¹⁷R^{17′}; wherein
m is 0, 1, or 2;
R¹⁷ is propynyl; and
R^{17′} is aminocarbonyl or carboxy; and
R^{a} is hydrogen or methyl; or R¹ and R^{a}, together with the atoms to which they are attached, form a pyrrolidine or piperazine ring, wherein each ring is optionally substituted with an amino group.

24. The compound of claim 23, or the pharmaceutically acceptable salt thereof, wherein
R¹ is selected from C₁-C₄alkyl, C₁-C₄alkylcarbonylaminoC₂-C₄alkyl, aminoC₁-C₃alkyl, aminocarbonylC₁-C₂alkyl, carboxypropyl, cyanomethyl, heteroarylmethyl, heterocyclylmethyl, hydroxyC₂alkyl; methoxyC₁-C₂alkyl, methylaminoC₁-C₂alkyl, NH₂C(NH)NHpropyl, and H₂NC(NH)piperidinyl; arylC₁-C₂alkyl; wherein the aryl part of the arylC₁-C₂alkyl is optionally substituted with one, two, or three groups independently selected from aminocarbonyl, carboxy, carboxymethoxy, halo, methoxy, methylcarbonylamino, propynyloxy, and trifluoromethyl;
R^{1′} is hydrogen;
R² is selected from arylC₁-C₂alkyl and heteroarylC₁-C₂alkyl, wherein the aryl part of the arylC₁-C₂alkyl and the heteroaryl part of the heteroarylC₁-C₂alkyl are optionally substituted with one or more groups independently selected from amino, aminocarbonyl, aminomethyl, carboxy, carboxymethyl, carboxymethoxy, cyano, halo, hydroxy, methoxy, methyl, nitro, and propynyloxy;
R^{2′} is hydrogen or methyl;
R³ is selected from aminocarbonylmethyl, carboxymethyl, and tetrazolyl;
R⁴ is selected from arylmethyl and heteroarylmethyl; wherein the aryl part of the arylmethyl and the heteroaryl part of the heteroarylmethyl are optionally substituted with one, two, three, four, or five groups independently selected from amino, halo, hydroxy, methoxy, methyl, and trifluoromethyl;
R⁵ is selected from C₁-C₅alkyl, arylmethyl, C₃-C₆cycloalkyl, (C₃-C₆cycloalkyl)methyl, hydroxyC₂alkyl, methoxymethyl, and phenyl; wherein the aryl part of the arylmethyl is optionally substituted with one, two, three, four, or five groups independently selected from aminomethyl, aminocarbonyl, carboxy, carboxymethoxy, hydroxy, and propynyloxy;
R⁶ is biphenylmethyl;
R⁷ is selected from C₁-C₄alkyl, aminoC₁-C₄alkyl, aminocarbonylethyl, aminocarbonylmethyl, arylmethyl, butynyl, carboxyethyl, C₃-C₆cycloalkyl, heteroarylmethyl, hydroxyC₂alkyl, methylcarbonylaminobutyl, phenyl, and NH₂C(X)NHC₂-C₄alkyl, wherein X is O or NH; wherein the aryl part of the arylmethyl is optionally substituted with one, two, or three groups independently selected from carboxy, carboxymethoxy, hydroxy, propynyloxy, and trifluoromethoxyl;
R⁸ is selected from C₁-C₄alkyl, C₁-C₄alkylcarbonylaminoC₂-C₄alkyl, aminoC₁-C₄alkyl, aminocarbonylethyl, carboxypropyl, hydroxymethyl, and imidazolylmethyl;
R^{8′} is hydrogen;
R⁹ is selected from C₁-C₄alkyl, cyclohexylmethyl, and phenylmethyl; wherein the phenyl part of the phenylmethyl is optionally substituted with one or two hydroxy groups;
R¹⁰ is selected from aminoC₁-C₄alkyl, aminocarbonylC₁-C₂alkyl, butylcarbonylaminoethyl, butynyl, carboxyC₁-C₃alkyl, hydroxyC₁-C₂alkyl, imidazolylmethyl, and NH₂C(NH)NHmethyl;
R¹¹ is selected from butyl, cyclohexylmethyl, and phenylmethyl; wherein the phenyl part of the phenylmethyl is optionally substituted with one, two, or three groups independently selected from fluoro and methyl;
R¹² is selected from C₃-C₄alkyl, carboxybutyl, hydroxyC₁-C₃alkyl, and phenylmethyl; wherein the phenyl part of the phenylmethyl is optionally substituted with one, two, or three propynyloxy groups;
R¹³ is selected from aminoC₁-C₄alkyl, C₁-C₄alkylcarbonylaminoC₂-C₄alkyl, aminocarbonylC₁-C₂alkyl, arylmethyl. butyl, carboxyC₁-C₂alkyl, heteroarylmethyl, hydroxyC₁-C₃alkyl, and NH₂C(X)NHpropyl, wherein X is O or NH; wherein the aryl part of the arylmethyl is optionally substituted with one, two, or three propynyloxy groups;
R¹⁴ is aminocarbonyl or —C(O)NR^{14′}CR¹⁵R^{15′}R^{15″}, wherein
R^{14′} is hydrogen or methyl;
R¹⁵ is selected from hydrogen, C₁-C₂alkyl, aminoC₁-C₄alkyl, aminocarbonylmethyl, butylcarbonylaminoethyl, carboxy, carboxyethyl, hydroxymethyl, NH₂C(NH)NHpropyl, and propynyl;
R^{15′} is hydrogen; methyl; or R¹⁵ and R^{15′}, together with the atoms to which they are attached, form a cyclopropyl ring; and
R^{15″} is hydrogen, aminocarbonyl, carboxy, or —(CH₂)ₙC(O)NHCHR¹⁶R^{16′}; wherein
n is 0, 1, or 2;
R¹⁶ is selected from hydrogen, aminoC₁-C₄alkyl, arylmethyl, carboxyethyl, heteroaryl, heteroarylethyl, hydroxymethyl, methoxymethyl, NH₂C(NH)NHpropyl, and propynyl; wherein the aryl part of the arylmethyl and the heteroaryl are optionally substituted with one or more groups independently selected from arylmethoxy and methyl;
R^{16′} is hydrogen, aminocarbonyl, carboxy, methyl, or —(CH₂)ₘC(O)NHCHR¹⁷R^{17′}; wherein
m is 0, 1, or 2; wherein
R¹⁷ is propynyl; and
R^{17′} is aminocarbonyl; and
R^{a} is hydrogen or methyl; or R¹ and R^{a}, together with the atoms to which they are attached, form a pyrrolidine ring, wherein the pyrrolidine ring is optionally substituted with an amino group.

25. The compound of claim 24, or the pharmaceutically acceptable salt thereof, wherein
R¹ is selected from C₂-C₄alkyl, aminoC₁-C₂alkyl, aminocarbonylmethyl, heteroarylmethyl, hydroxyC₂alkyl, morpholinylmethyl, NH₂C(NH)NHpropyl, and phenylmethyl; wherein the phenyl part of the phenylmethyl is optionally substituted with one, two, or three groups independently selected from carboxymethoxy and fluoro;
R^{1′} is hydrogen;
R² is selected from phenylmethyl and pyridylmethyl, and wherein the phenyl part of the phenylmethyl are optionally substituted with one, two, or three groups independently selected from hydroxy, carboxy, and carboxymethoxy;
R^{2′} is hydrogen;
R³ is carboxymethyl;
R⁴ is selected from indolylmethyl and phenylmethyl; wherein the phenyl part of the phenylmethyl is optionally substituted with one, two, or three groups independently selected from hydroxy and methyl;
R⁵ is selected from phenylmethyl and propyl; wherein the phenyl part of the phenylmethyl is optionally substituted with one, two, or three groups independently selected from carboxy, carboxymethoxy, hydroxy, and propynyloxy; and
R⁶ is biphenylmethyl;
R⁷ is selected from C₃-C₄alkyl, NH₂C(O)NHpropyl, and phenylmethyl; wherein the phenyl part of the phenylmethyl is optionally substituted with one, two, or three groups independently selected from carboxy, carboxymethoxy, hydroxy, and propynyloxy;
R⁸ is selected from aminopropyl and methyl;
R^{8′} is hydrogen;
R⁹ is isobutyl;
R¹⁰ is aminoethyl;
R¹¹ is selected from butyl and cyclohexylmethyl;
R¹² is selected from hydroxyisopropyl, hydroxypropyl, isopropyl, and propyl;
R¹³ is selected from aminopropyl, carboxyethyl, hydroxyC₁-C₂alkyl, imidazolylmethyl, and phenylmethyl; wherein the phenyl part of the phenylmethyl is optionally substituted with one, two, or three propynyloxy groups;
R¹⁴ is aminocarbonyl or —C(O)NR^{14′}CR¹⁵R^{15′}R^{15″}, wherein
R^{14′} is hydrogen;
R¹⁵ is selected from aminocarbonylmethyl, aminoethyl, and methyl;
R^{15′} is hydrogen; and
R^{15″} is hydrogen, aminocarbonyl, or —(CH₂)ₙC(O)NHCHR¹⁶R^{16′}; wherein
n is 0 or 1;
R¹⁶ is propynyl; and
R^{16′} is hydrogen, aminocarbonyl, or carboxy; and
R^{a} is hydrogen.

26. A pharmaceutical composition comprising a compound of any one of claims 1 to 25, or a pharmaceutically acceptable salt thereof.

27. A method of enhancing, stimulating, and/or increasing an immune response in a subject in need thereof, wherein the method comprises administering to the subject a therapeutically effective amount of a compound of any one of claims 1 to 25, or a pharmaceutically acceptable salt thereof.

28. A method of blocking the interaction of PD-1 with PD-L1 in a subject, wherein the method comprises administering to the subject a therapeutically effective amount of a compound of any one of claims 1 to 25 or a pharmaceutically acceptable salt thereof.
